(19) United States
(12) Patent Application Publication
(10) Pub. No.: US 2022/0158095 A1
(43) Pub. Date: May 19, 2022

(54) Compound, Organic Electroluminescent Device Containing Same and Application Thereof

(71) Applicant: BEIJING ETERNAL MATERIAL TECHNOLOGY CO., LTD, Beijing (CN)

(72) Inventors: Jinhua HUANG, Beijing (CN); Lichang ZENG, Beijing (CN); Weihong ZHANG, Beijing (CN)

(21) Appl. No.: 17/607,035

(22) PCT Filed: Apr. 7, 2020
(86) PCT No.: PCT/CN2020/083499

§ 371 Date: Oct. 28, 2021

(30) Foreign Application Priority Data
Apr. 30, 2019 (CN) 201910364366.X
Aug. 27, 2019 (CN) 201910796244.8
Sep. 10, 2019 (CN) 201910857132.9
Dec. 31, 2019 (CN) 201911423824.9

(51) Int. Cl. H01L 51/00; C07C 211/61; C07D 307/91; C07D 333/76; C07D 209/86; C07C 211/58

(52) U.S. Cl. CPC H01L 51/006; C07C 211/61; C07D 307/91; H01L 51/0061; C07D 333/76; H01L 51/5056; C07C 211/58; H01L 51/0073; H01L 51/0074; H01L 51/0072; H01L 51/0058; C07D 209/86

(57) ABSTRACT

A compound, an organic electroluminescent device containing the compound, and an application thereof. The compound has a structure shown in (I).

### CROSS-REFERENCE TO RELATED APPLICATION

The present application is a National Stage of International Patent Application No. PCT/CN2020/083499 filed on Apr. 7,2020, which claims the benefit of priority to Chinese Patent Application Nos. 201910364366.X, filed on Apr. 30, 2019, 201910796244.8, filed on Aug. 27, 2019, 201910857132.9, filed on Sep. 10, 201911423824.9, filed on Dec. 31,2019, the disclosures of which are incorporated herein by reference in their entirety.

### TECHNICAL FIELD

The present invention relates to the field of organic luminescent compounds and organic electroluminescent devices, and in particular to a compound, an organic electroluminescent device containing the same and an application thereof.

### BACKGROUND

In recent years, optoelectronic devices based on organic materials have become more and more popular. With inherent flexibility, organic materials are very suitable for manufacture on a flexible substrate. Beautiful and fascinating optoelectronic products may be designed and produced according to demands, thus achieving incomparable advantages relative to inorganic materials. Examples of such kind of organic optoelectronic device includes organic light emitting diodes (OLED), organic field effect transistors, organic photovoltaic cells, organic sensors and the like. OLED has been developed rapidly particularly, and has achieved commercial success in information display field. OLED may provide three colors with high saturability, i.e. red, green and blue; and the full-color display device made of OLED requires no extra backlight, and has the advantages such as, dazzling color, lightness, and softness.

With the constant promotion of OLED in two major fields of illumination and display, people pay more attention to the studies on the core materials thereof. This is because an OLED device with good efficiency and long service life is an optimization result of device structures and various organic materials. To prepare an OLED luminescent device with lower voltage, better luminous efficiency and longer service life and to achieve the continuous promotion of OLED device performances, researchers not only need to make innovations on the structure and manufacturing process of OLED device, but also need to make constant research and innovations on photoelectric functional materials in OLED device, thereby preparing functional materials with higher performances. In view of this, OLED material industry has been devoted to the development of a novel organic electroluminescent material, thus achieving low starting voltage, high luminous efficiency and more excellent service life of the device.

At present, people have developed multiple organic materials in combination with various peculiar device structures, which may promote carrier mobility, regulate and control carrier balance, break through electroluminescent efficiency and delay device attenuation. Due to quantum mechanics, common fluorescent luminophors give out light mainly by means of singlet exciton produced by the combination of electrons and holes, which is still applied in various OLED products widely. Some metal complexes, e.g., iridium complex, may simultaneously make use of triplet exciton and singlet exciton for luminescence, called phosphorescence luminophors; and the energy conversion efficiency may be promoted up to four times relative to the conventional fluorescent luminophor. Thermally activated delayed fluorescence (TADF) technology may still effectively make use of triplet exciton to achieve higher luminous efficiency by promoting the transformation to singlet exciton from triplet exciton without a metal complex. Thermally activated sensitized fluorescence (TASF) technology utilizes a material having TADF properties to sensitize a luminophor by a way of energy transfer, which may similarly achieve higher luminous efficiency. However, phosphorescent host materials still have a greater room for improvement in luminescence property, for example, carrier transport capacity.

As OLED products are gradually put into the market, people are increasingly demanding for higher performances of such products. The OLED materials and device structures in the arts may not completely solve various aspects of problems, such as OLED product efficiency, service life and cost. Therefore, it is urgent to develop more various types of OLED materials having higher performances in the field, thereby promoting the device performances.

### SUMMARY

### Problems to be Solved by the Present Invention

As mentioned above, the existing OLED materials and device structures are increasingly unable to meet people's demands in various aspects, such as efficiency, service life and cost of the OLED device. Therefore, people are expecting to develop a novel compound, capable of being applied in OLED device and promoting device performances.

With a view to the study of novel OLED materials, the inventor of the present application develops an excellent material suitable for a hole transport layer or an electron blocking layer. Specifically, the objective of the present invention is to provide a compound, an organic electroluminescent device comprising the same and an application thereof. The compound may improve and balance the migration rate of holes in OLED device. The OLED device manufactured on the basis of the compound of the present invention has a low starting voltage, a high luminous efficiency and more excellent service life, and may satisfy the current panel manufacturing enterprises' demands for high performance materials.

### Solution to Solve the Problems

The inventor is concentrated on studies to find that the control of a “naphthalene-triaryl amine” structure may effectively regulate and control the triplet-state energy level of a target molecule, thus obtaining a novel hole-transport material with good hole transport performance and high triplet-state energy level. The “naphthalene-triaryl amine” mentioned herein refers to tri-“aryl” amine containing a naphthalene ring structure directly linked to nitrogen; the “aryl” here is used in a general sense, and includes heteroaryl, fused-cyclic aryl, fused-cyclic heteroaryl, and these three “aryl” groups may be directly linked to the central nitrogen atom of the “naphthalene-triaryl amine”, and also may be linked via a linking group.

Further, the inventor finds that in the “naphthalene-triaryl amine”, if there is specific substituent in the ortho position of diarylamido on the naphthalene ring or, one aryl in the “tri-“aryl” amine” is binaphthylyl (namely, there is a substituted or unsubstituted naphthyl group on the naphthalene ring), and the other aryl is substituted or unsubstituted benzodimethyl fluorenyl, and the third aryl is a specific substituent, the target molecule has a suitable triplet-state energy level. The above specific substituent refers to a substituted or unsubstituted C₆-C₃₀ aryl or a substituted or unsubstituted C₃-C₃₀ heteroaryl.

The present invention provides a compound, characterized by having a structure as shown in Formula (I):

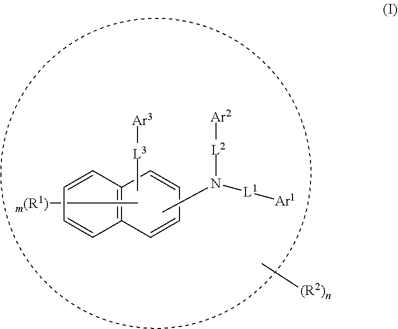

where, Ar¹ and Ar² are each independently selected from H, a substituted or unsubstituted C₆-C₅₀ aryl, a substituted or unsubstituted C₃-C₃₀ heteroaryl, a substituted or unsubstituted C₆-C₅₀ fused aryl, a substituted or unsubstituted C₃-C₃₀ fused heteroaryl; and when Ar¹ is H, L¹ is not a single bond; when Ar² is H, L² is not a single bond; Ar³ is selected from a substituted or unsubstituted C₆-C₃₀ aryl, a substituted or unsubstituted C₃-C₃₀ heteroaryl, a substituted or unsubstituted C₆-C₅₀ fused aryl, and a substituted or unsubstituted C₃-C₃₀ fused heteroaryl;

L¹-L³ are each independently selected from a single bond, a substituted or unsubstituted C₁-C₁₀ alkylene, a substituted or unsubstituted C₆-C₅₀ arylene, and a substituted or unsubstituted C₃-C₃₀ heteroarylene group;

m is an integer of 0-6, and n is an integer of 0-15;

R¹ is each independently selected from H, a halogen, carbonyl, carboxyl, amino, amido, cyano, nitryl, an ester group, hydroxyl, silicyl, a substituted or unsubstituted C₁-C₂₀ alkyl, a substituted or unsubstituted C₃-C₂₀ cycloalkyl, a substituted or unsubstituted C₂-C₂₀ alkenyl, a substituted or unsubstituted C₂-C₂₀ alkynyl, a substituted or unsubstituted C₁-C₂₀ alkoxy, a substituted or unsubstituted C₃-C₁₀ cycloalkoxy, a substituted or unsubstituted C₆-C₅₀ aryl, a substituted or unsubstituted C₃-C₃₀ heteroaryl, and a C₆-C₅₀ fused aryl;

R² is, on each occurrence, a substituent of Ar¹-Ar³, L¹-L³, R¹ or the naphthalene ring in the Formula (I), a substituent of Ar¹-Ar³, L¹-L³, R¹ and a substituent on a naphthalene ring in the Formula (I), each independently selected from H, a halogen, carbonyl, carboxyl, cyano, nitryl, an ester group, hydroxyl, amido, a C₁-C₁₀ silicyl, a substituted or unsubstituted C₁-C₂₀ alkyl, a substituted or unsubstituted C₃-C₂₀ cycloalkyl, a C₂-C₁₂ alkenyl, a C₂-C₁₂ alkynyl, a substituted or unsubstituted C₁-C₁₂ alkoxy, a substituted or unsubstituted C₃-C₁₀ cycloalkoxy, a substituted or unsubstituted C₆-C₅₀ aryl, a substituted or unsubstituted C₃-C₃₀ heteroaryl, and a C₆-C₅₀ fused aryl;

the group

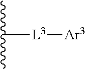

is located in an ortho position of the group

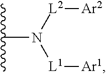

and neither R¹ nor R² is amido; or Ar¹ is a substituted or unsubstituted C₆-C₃₀ aryl or a substituted or unsubstituted C₃-C₃₀ heteroaryl, Ar² is substituted or unsubstituted benzodimethyl fluorenyl, and Ar³ is substituted or unsubstituted naphthyl;

when each substituted or unsubstituted group has a substituent, the substituent is selected from one or more of a halogen, cyano, nitryl, an ester group, hydroxyl, carbonyl, carboxyl, cyano, amido, a C₁-C₁₀ silicyl, a C₁-C₂₀ alkyl, a C₃-C₂₀ cycloalkyl, a C₂-C₂₀ alkenyl, a C₂-C₁₀ alkynyl, a C₁-C₂₀ alkoxy or thioalkoxy, a C₆-C₃₀ arylamino, a C₃-C₃₀ heteroarylamino, a C₆-C₃₀ monocyclic or fused-cyclic aryl, a C₃-C₃₀ monocyclic or fused-cyclic heteroaryl.

The compound of the present invention as mentioned above is a tri-“aryl” amine containing a naphthalene ring structure directly linked to nitrogen. There is a substituted or unsubstituted C₆-C₃₀ aryl group or substituted or unsubstituted C₃-C₃₀ heteroaryl group in an ortho position of diarylamido; or, one aryl in the “tri-“aryl” amine” is binaphthylyl; the other aryl is substituted or unsubstituted benzodimethyl fluorenyl, and the third aryl is substituted or unsubstituted C₆-C₃₀ aryl or substituted or unsubstituted C₃-C₃₀ heteroaryl. The compound of the present invention as mentioned above has good hole transport performance, and high triplet-state energy level and thus, is suitable for being used as a hole-transport material.

It should be indicated that in this description, the Ca-Cb means of expression represents that the group has a carbon number of a-b. Unless otherwise stated, the carbon number is exclusive of the carbon number of the substituent thereof. The scope of carbon number also represents that the carbon number of the group may be any integer within the range of value. In this present invention, the expression of chemical elements contains the concept of the isotopes having the same chemical properties, for example, the expression of “H”, also contains the concept of “deuterium” and “tritium” having the same chemical properties.

In this present invention, the means of expression that “ [symbol] ” is not linked on a ring, but lines across the ring structure represents that a linking site may be in any bondable position on the benzene ring.

In this present invention, unless otherwise stated specifically, aryl and heteroaryl respectively refer to monocyclic aryl and monocyclic heteroaryl.

In this present invention, the carbon number in the substituted or unsubstituted C₆-C₅₀ aryl or fused aryl, for example, may be 6, 8, 10, 12, 14, 15, 16, 18, 20, 23, 25, 26, 28, 30, 33, 35, 38, 40, 45, 50, and the like. Unless otherwise stated specifically, the substituted or unsubstituted C₆-C₅₀ aryl or fused aryl is preferably, C₆-C₃₀ aryl or fused aryl, more preferably, a radical group in a group consisting of phenyl, biphenyl, terphenylyl, naphthyl, anthryl, phenanthryl, indenyl, fluorenyl and a derivative thereof, fluoranthryl, triphenylene, pyrenyl, perylenyl, chrysenyl, and naphthacenyl. Specifically, the biphenyl is selected from 2-biphenyl, 3-biphenyl and 4-biphenyl; terphenyl includes p-tribiphenyl-4-yl, p-tribiphenyl-3-yl, p-tribiphenyl-2-yl, m-tribiphenyl-4-yl, m-tribiphenyl-3-yl, and m-tribiphenyl-2-yl; the naphthyl includes 1-naphthyl and 2-naphthyl; the anthryl is selected from 1-anthryl, 2-anthryl and 9-anthryl; the fluorenyl is selected from 1-fluorenyl, 2-fluorenyl, 3-fluorenyl, 4-fluorenyl and 9-fluorenyl; the fluorenyl derivative is selected from 9,9′-dimethyl fluorenyl, 9,9′-spirobifluorenyl and benzofluorenyl; the pyrenyl is selected from 1-pyrenyl, 2-pyrenyl and 4-pyrenyl; the naphthacenyl is selected from 1-naphthacenyl, 2-naphthacenyl and 9-naphthacenyl. Unless otherwise stated specifically, it is preferably phenyl, biphenyl, naphthyl, anthryl, phenanthryl, fluorenyl, and the like, and more preferably, phenyl and naphthyl, more preferably, phenyl.

In this description, C₆-C₅₀ arylene is obtained by removing a hydrogen on the basis of the above C₆-C₅₀ aryl. Unless otherwise stated specifically, the carbon number and preferred embodiments of the C₆-C₅₀ arylene correspond to those of the above C₆-C₅₀ aryl (removing a hydrogen). As detailed examples of C₆-C₅₀ arylene, phenylene, naphthylene and the like may be cited as an example.

In this description, the heteroatom usually refers to an atom or a radical selected from N, O, S, P, Si and Se, preferably, N, O, S, more preferably, N. The heteroaryl mentioned in this description refers that at least one carbon-ring atom in aryl is substituted by a heteroatom.

In this description, the carbon number of the substituted or unsubstituted C₃-C₃₀ heteroaryl or fused heteroaryl, for example, may be 3, 5, 6, 8, 10, 12, 14, 15, 16, 18, 20, 23, 25, 26, 28, 30, and the like. Unless otherwise stated specifically, the substituted or unsubstituted C₃-C₃₀ heteroaryl or fused heteroaryl is preferably C₄-C₂₀ heteroaryl or fused heteroaryl, more preferably nitrogen-bearing heteroaryl or fused heteroaryl, oxygen-bearing heteroaryl or fused heteroaryl, sulfur-bearing heteroaryl or fused heteroaryl; detained examples may be cited as follows: furyl, thienyl, pyrryl, bipyridyl, benzofuryl, benzothienyl, isobenzofuryl, indolyl, quinolyl, dibenzofuryl, dibenzothienyl, carbazolyl and a derivative thereof, where, the carbazolyl derivative thereof is preferably 9-phenylcarbazole, 9-naphthylcarbazole benzocarbazole, dibenzocarbazole, or indolocarbazole. Unless otherwise stated specifically, it is preferably pyridyl, quinolyl, dibenzofuryl, dibenzothienyl, and more preferably pyridyl.

In this description, the C₃-C₃₀ heteroarylene is obtained by removing an H on the basis of the above C₃-C₃₀ aryl. Unless otherwise stated specifically, the carbon number and preferred embodiments of the C₃-C₃₀ heteroarylene correspond to those of the above C₃-C₃₀ heteroaryl (removing a hydrogen). As detailed examples of C₃-C₃₀ heteroarylene, pyridylidene, pyrrylidene and the like may be set as an example.

In this description, the alkyl refers to chain-typed alkyl which may be linear alkyl or branched alkyl. The carbon number of the C₁-C₂₀ chain-typed alkyl may be 1, 2, 3, 4, 5, 6, 7, 8, 9, 10, 12, 15, 18, 20, and the like. Unless otherwise stated specifically, the C₁-C₂₀ chain-typed alkyl is preferably C₁-C₁₀ chain-typed alkyl, more preferably C₁-C₆ chain-typed alkyl. Examples of the chain-typed alkyl may be cited as follows: methyl, ethyl, n-propyl, n-butyl, n-hexyl, n-octyl, isopropyl, isobutyl, tert-butyl, and the like. Unless otherwise stated specifically, alkyl is preferably methyl, ethyl, n-propyl, isopropyl, more preferably, methyl.

In this description, alkylene refers to chain-typed alkylene which may be linear alkylene or contain branched alkylene. Unless otherwise stated specifically, in this description, C₁-C₁₀ alkylene may be obtained by removing a hydrogen on the basis of the above C₁-C₁₀ chain-typed alkyl. Examples of C₁-C₁₀ alkylen may be cited as follows: methylene, ethylidene, propylidene, and the like.

In this description, the carbon number of the C₃-C₂₀ cycloalkyl may be 4, 5, 6, 7, 8, 9, 10, and the like. Examples of C₃-C₂₀ cycloalkyl may be cited as follows: cyclobutyl, cyclopentyl, cyclohexyl, cyclooctyl, and the like.

In this description, the carbon number of the C₃-C₂₀ alkenyl, may be, for example, 2, 3, 4, 5, 6, 7, 8, 9, 10, and the like. Examples of the C₂-C₂₀ alkenyl may be cited as follows: vinyl, propenyl, 1-butenyl, and the like; the carbon number in the C₂-C₂₀ alkenyl may be, for example, 2, 3, 4, 5, 6, 7, 8, 9, 10, and the like. Examples of the C₂-C₂₀ alkynyl may be cited as follows: acetenyl, propinyl, 1-butynyl, and the like.

In this description, the carbon number of the C₁-C₂₀ alkoxy may be 2, 3, 4, 5, 6, 7, 8, 9, 10, and the like. Examples of the C₁-C₂₀ alkoxy may be cited as follows: groups obtained by linking the above C₂-C₂₀ chain-typed alkyl to —O—, for example, methoxy, ethyoxyl, propoxy, butoxy, pentyloxy, hexyloxy, heptyloxy, octyloxy, nonyloxy, decyloxy, undecyloxy, dodecyloxy, and the like, preferably, methoxy, ethyoxyl, propoxy, and more preferably, methoxy.

In this description, the carbon number in the C₃-C₁₀ cycloalkoxy may be 4, 5, 6, 7, 8, 9, 10, and the like. Examples of C₃-C₁₀ cycloalkoxy may be cited as follows: radical groups obtained by linking the above C₃-C₃₀ cycloalkyl to —O—, for example, cyclobutoxy, cyclopentyloxy, cyclohexyloxy, cyclooctyloxy, and the like.

In this description, examples of the C₁-C₂₀ thioalkoxy may be cited as follows: radical groups obtained by substituting O in the above C₁-C₂₀ alkoxy with S, for example, methylthio, thiooctyloxy (octylthio), and the like.

In this description, examples of the halogen may be cited as follows: fluorine, chlorine, bromine, iodine, and the like, and preferably fluorine unless otherwise stated specifically. In this description, unless otherwise stated specifically, the amino refers to a —NH₂ group; amido refers to a group obtained by substituting at least one H in amino with an organic group (namely, N is directly linked to C), including alkylamino, arylamino, heteroarylamino, or the like. The carbon number in the C₆-C₃₀ arylamino may be 10, 12, 14, 16, 18, 20, 26, 28, and the like. Examples of C₆-C₃₀ arylamino may be cited as follows: groups obtained by linking the above C₆-C₃₀ aryl to —NH—, for example, phenylamino, naphthylamino, and the like. The carbon number in the C₃-C₃₀ heteroarylamino may be 6, 8, 10, 12, 14, 16, 18, 20, 26, 28, and the like. Examples of the C₃-C₃₀ heteroarylamino may be cited as follows: groups obtained by linking the above C₃-C₃₀ heteroaryl to —NH—, for example, pyridylamino, pyrrylamino, and the like.

In this description, examples of the C₁-C₁₀ silicyl may be cited as follows: methylsilicyl, trimethylsilicyl, triethylsilicyl, and the like.

Based on the above compound of the present invention, the structure thereof (type of substituents, linking site and the like) may be further defined to obtain a compound having more excellent performance. Three preferred embodiments will be described below.

### Preferred Embodiment I

The compound of the present invention preferably has a structure as shown in Formula (I):

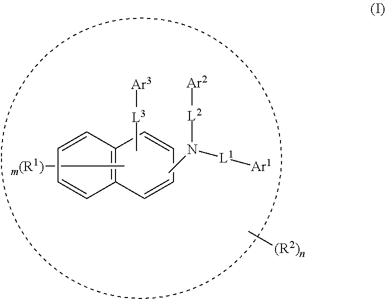

where, the group

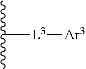

is located in an ortho position of the group

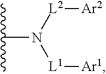

Ar¹-Ar³ are each independently selected from a substituted or unsubstituted C₆-C₃₀ aryl or a substituted or unsubstituted C₃-C₃₀ heteroaryl;

L¹-L³ are each independently selected from a single bond, a substituted or unsubstituted C₆-C₃₀ alkylene, and a substituted or unsubstituted C₆-C₃₀ heteroarylene group;

R¹ is independently selected from H, a C₁-C₂₀ chain-typed alkyl, a C₃-C₂₀ cycloalkyl, a C₂-C₂₀ alkenyl, and a C₂-C₂₀ alkynyl, a C₁-C₂₀ alkoxy, a halogen, cyano, nitryl, hydroxyl, silicyl, a substituted or unsubstituted C₆-C₃₀ aryl or a substituted or unsubstituted C₃-C₃₀ heteroaryl;

R² is, on each occurrence, a substituent of Ar¹-Ar³, L¹-L³, R¹ or the naphthalene ring in the Formula (I), each independently selected from one of H, a substituted or unsubstituted C₃-C₂₀ cycloalkyl; and at least one R² is selected from a substituted or unsubstituted C₃-C₂₀ cycloalkyl;

m is an integer of 1-6, and n is an integer of 1-15;

when each substituted or unsubstituted group has a substituent, the substituent is selected from one or a combination of more of a halogen, a C₁-C₂₀ alkyl, a C₃-C₂₀ cycloalkyl, a C₂-C₂₀ alkenyl, a C₁-C₂₀ alkoxy or thioalkoxy, a C₆-C₃₀ monocyclic or fused-cyclic aryl, a C₃-C₃₀ monocyclic or fused-cyclic heteroaryl.

The ortho position of the “naphthalene-triaryl amine” in the present invention has a specific aryl or heteroaryl substituent, which may efficiently up-regulate the triplet-state energy level of molecules. Meanwhile, a cycloalkyl group is brought into molecules to promote the arrangement of molecules in a spreading way, which improves the optical extraction efficiency while promoting the carrier transmission performance, thus promoting the photoelectric and life performance of the device.

Further. Ar³ is a substituted or unsubstituted C₁₀-C₃₀ fused-cyclic aryl or a substituted or unsubstituted C₆-C₃₀ fused-cyclic heteroaryl.

The above organic compound of the present invention may be specifically a structure as shown in the following (a) to (c):

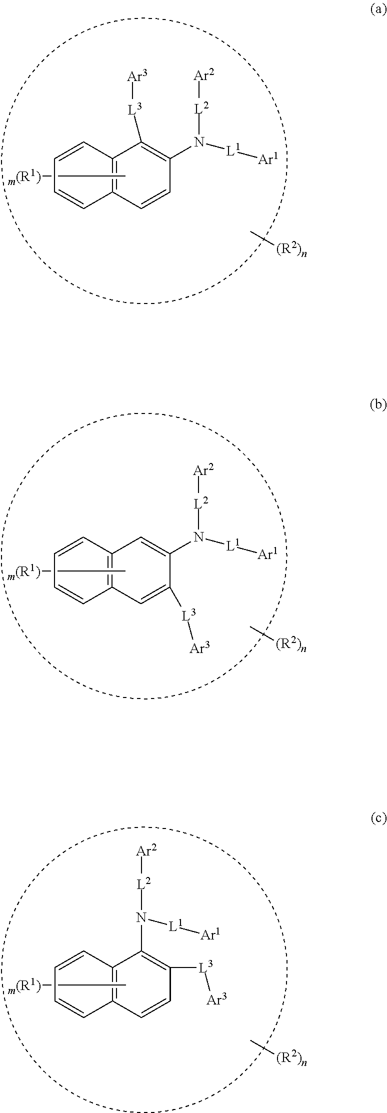

The above organic compound of the present invention preferably has a structure as shown in (A-1) to (A-3):

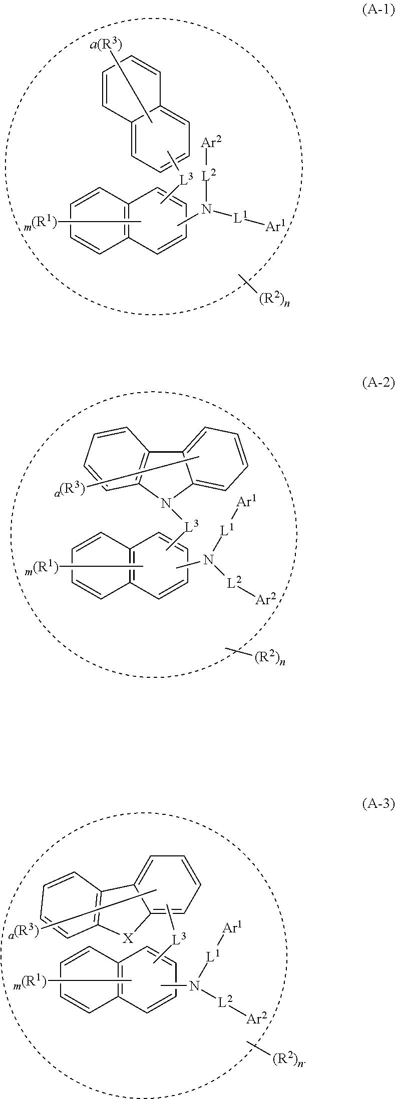

where, R³ is independently selected from one of H, a C₁-C₂₀ chain-typed alkyl, a C₃-C₂₀ cycloalkyl, a C₂-C₂₀ alkenyl, a C₂-C₂₀ alkynyl, a C₁-C₂₀ alkoxy, a halogen, cyano, nitryl, hydroxyl, silicyl, a substituted or unsubstituted C₆-C₃₀ aryl, a substituted or unsubstituted C₃-C₃₀ heteroaryl; X is O, S, NR⁴, CR⁵R⁶ or SiR⁷R⁸; R⁴-R⁸ are each independently selected from H, a C₁-C₂₀ chain-typed alkyl, a C₃-C₂₀ cycloalkyl, a substituted or unsubstituted C₆-C₃₀ aryl, a substituted or unsubstituted C₃-C₃₀ heteroaryl; R⁵ and R⁶ are preferably each independently selected from methyl; when the organic compound is the structure as shown in Formula (A-1), a is an integer of 1-7; when the organic compound is the structure as shown in Formula (A-2), a is an integer of 1-8; and when the organic compound is the structure as shown in Formula (A-3), a is an integer of 1-7. In other words, the above organic compound of the present invention preferably has such a structure: Ar³ is substituted or unsubstituted naphthyl, substituted or unsubstituted fluorenyl, or substituted or unsubstituted dibenzo-X hetercyclopentadiene; X is O, N, S, or Si.

The reason why the above preferred structure, as a hole-transport material, has more excellent performances has been not clear. It is presumed that planar molecule may be expanded when Ar³ of the naphthalene-triaryl amine is the preceding fused-cyclic; aryl or fused-cyclic, heteroaryl, beneficial to hole transport.

The above organic compound of the present invention preferably has a structure as shown in any one of

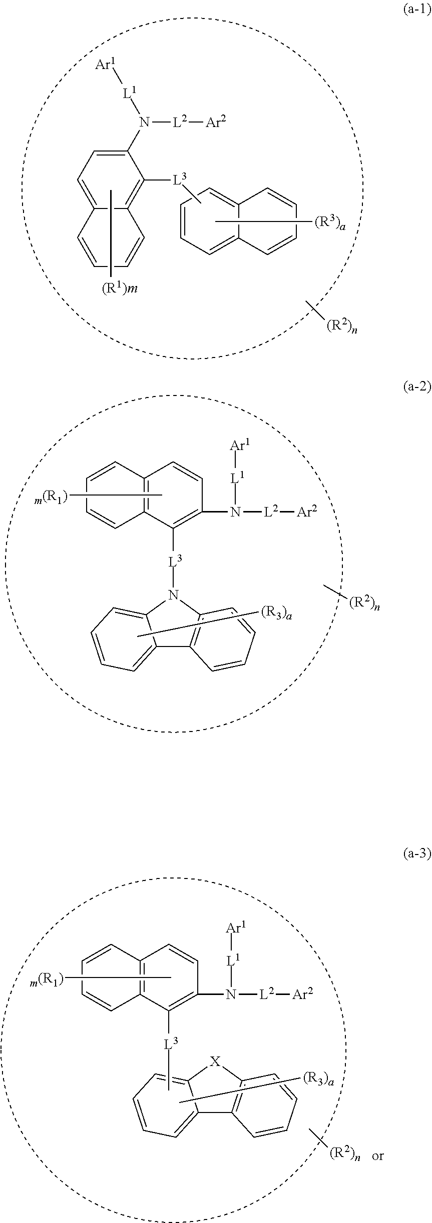

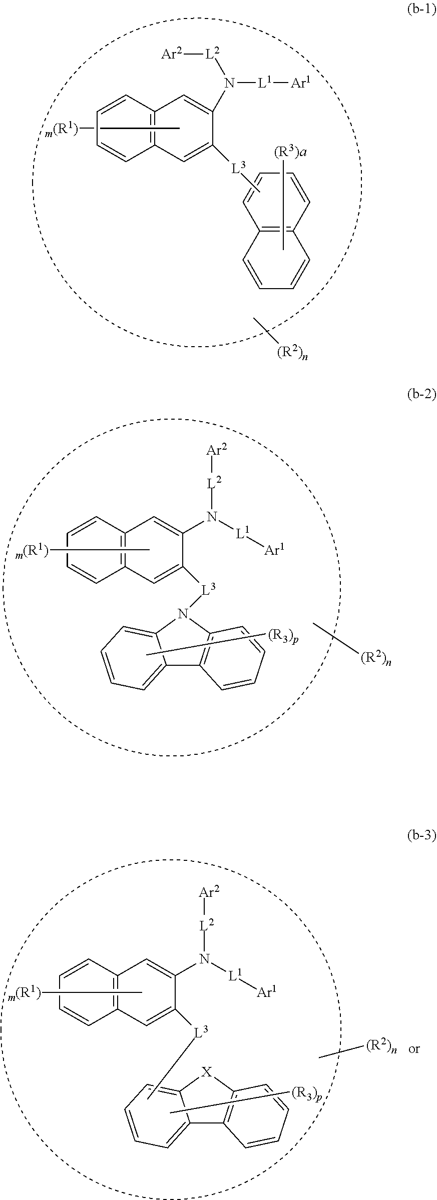

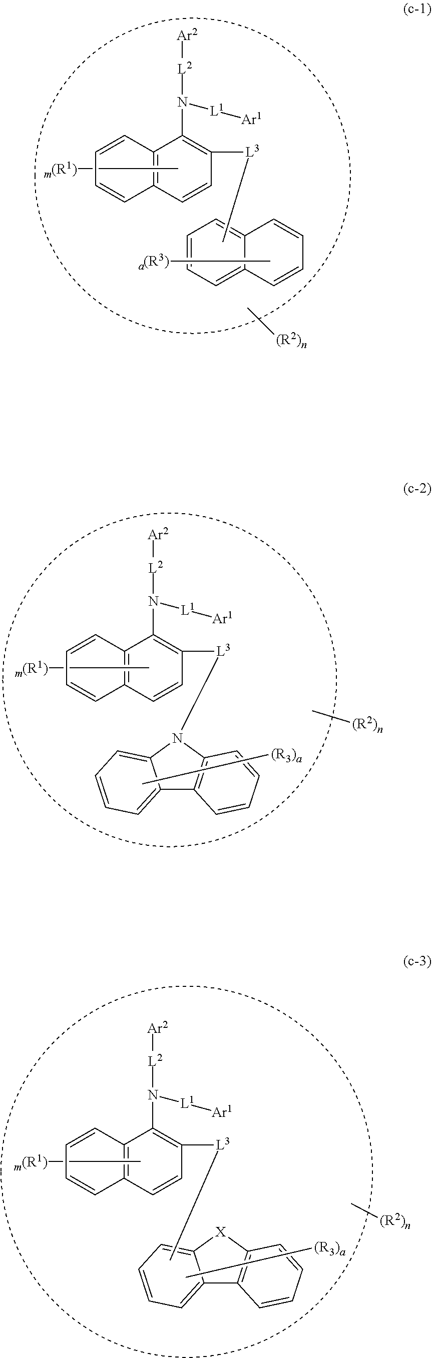

In other words, the above organic compound of the present invention is preferably, as follows: the group

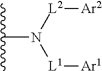

is located in a 1-position or 2-position on the naphthalene ring, and when the group

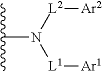

is located in the 1-position on the naphthalene ring, the group

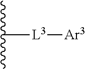

is located in the 2-position on the naphthalene ring.

In the above organic compound of the present invention, R² is each preferably and independently selected from one of the following structures:

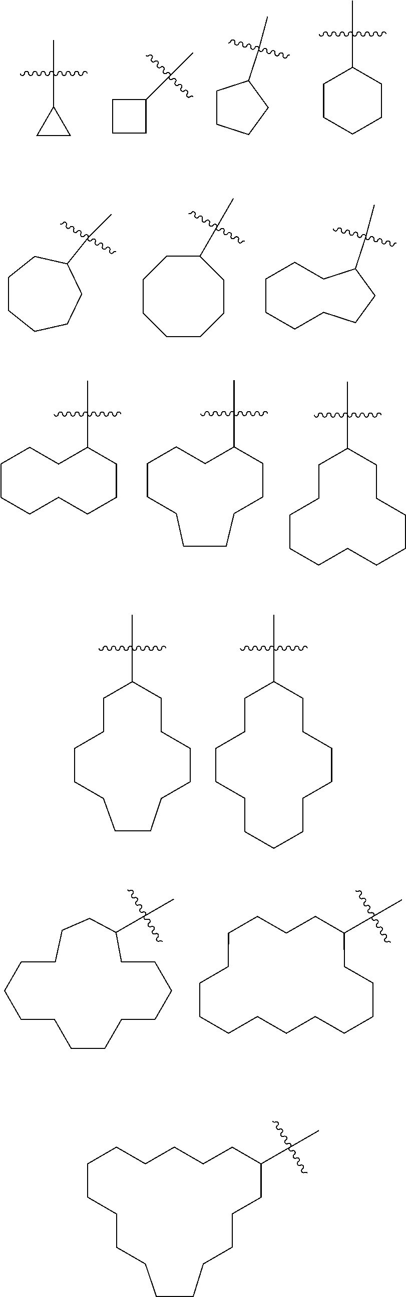

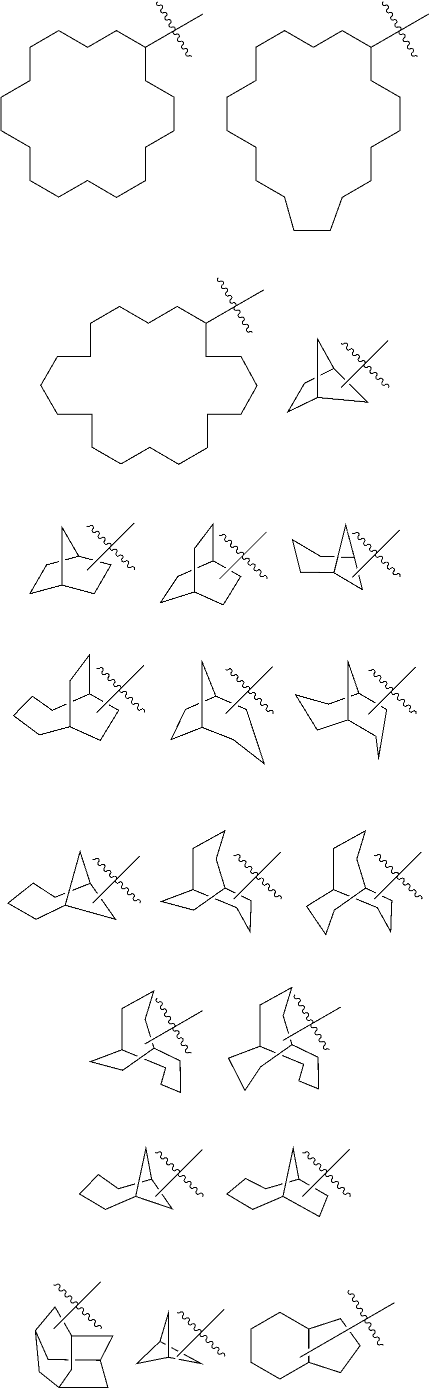

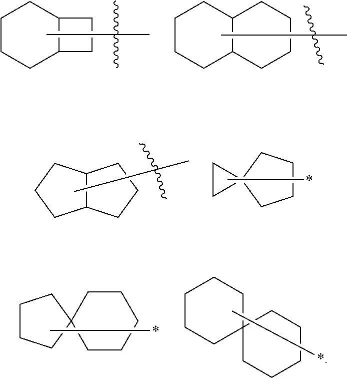

R² is more preferably, each independently selected from one of cyclopentyl, cyclohexyl and cycloheptyl.

In the above organic compound of the present invention, preferably, Ar¹ is substituted or unsubstituted C₁₀-C₃₀ fused-cyclic aryl or substituted or unsubstituted C₆-C₃₀ fused-cyclic heteroaryl; Ar² is substituted or unsubstituted C₆-C₃₀ non-fused-cyclic aryl or substituted or unsubstituted C₃-C₃₀ non-fused-cyclic heteroaryl. Moreover, the carrier transport performance may be also enhanced.

Ar¹ is selected from one of the following structures:

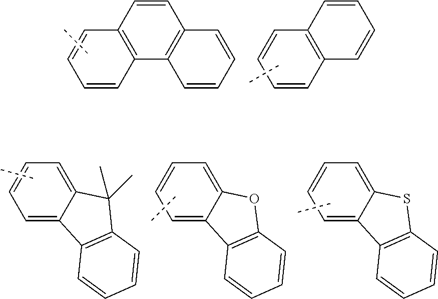

Ar² is selected from one of the following structures:

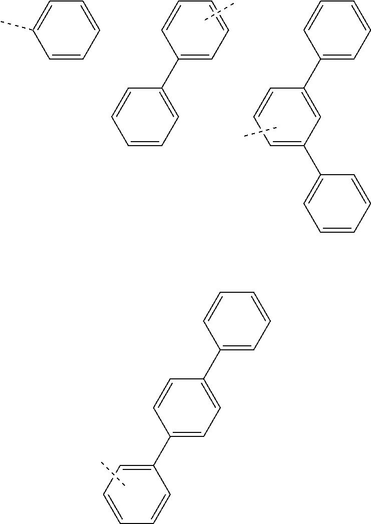

where, the dotted line denotes an access site of a group; the representing method of lining across the benzene ring with the dotted line denotes that a linking site of a group may be in any bondable position on the benzene ring.

In the above organic compound of the present invention, preferably, at least one of Ar¹ and Ar² has a substituent of substituted or unsubstituted C₃-C₂₀ cycloalkyl, which facilitates the adjustment of a space three-dimensional conformation, thus achieving the regulation and control of intermolecular distance. More preferably, Ar² has the substituent of substituted or unsubstituted C₃-C₂₀ cycloalkyl. The introduction of cycloalkyl on Ar² may effectively regulate and control the spatial form accumulation and molecular crystallinity of a target molecule, thus obtaining a novel hole-transport material with good hole transport performance, high triplet-state energy level and stable amorphous thin film.

In the above organic compound of the present invention, L¹ and L² are preferably, each independently selected from a single bond, phenylene or naphthylene, more preferably, L¹-L³ are a single bond. This is beneficial for the molecules to be piled more tightly, thus improving the hole transport performance.

The above organic compound of the present invention preferably has a structure as shown in the following P1-P291, but these compounds are merely representative.

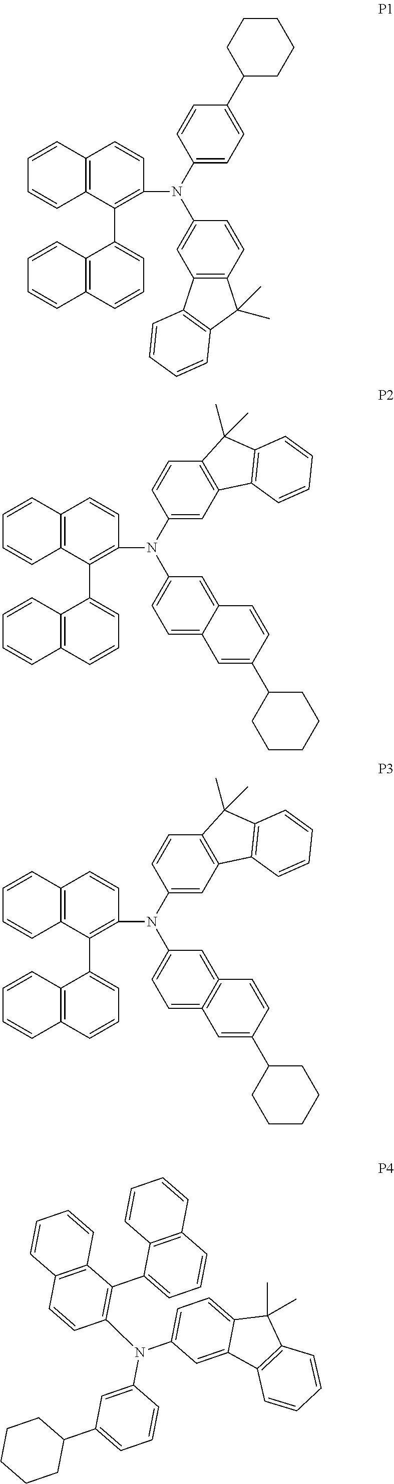

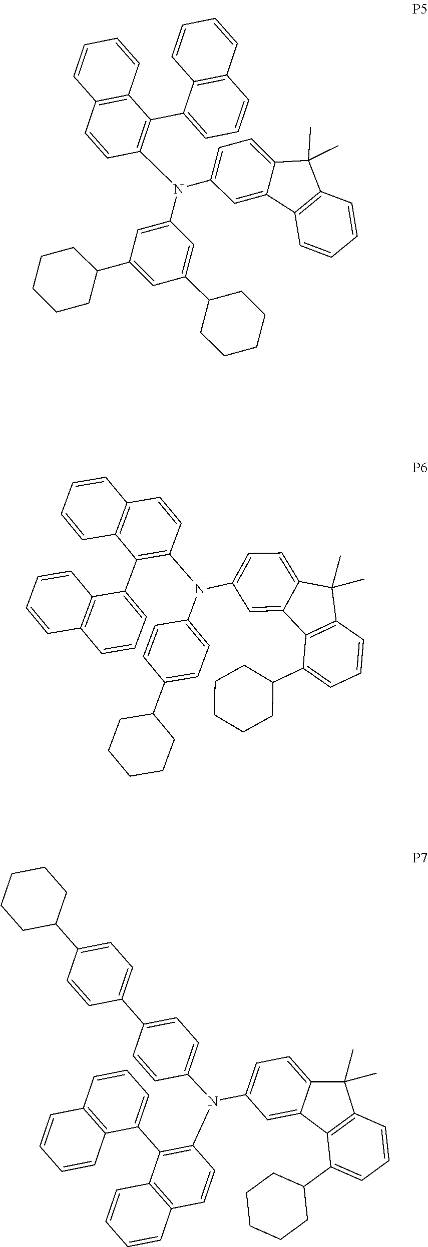 [SMILES: CC1(C)c2ccccc2-c2cc(N(c3cc(C4CCCCC4)cc(C4CCCCC4)c3)c3ccc4ccccc4c3-c3cccc4ccccc34)ccc21 ; CC1(C)c2ccc(N(c3ccc(C4CCCCC4)cc3)c3ccc4ccccc4c3-c3cccc4ccccc34)cc2-c2c(C3CCCCC3)cccc21 ; CC1(C)c2ccc(N(c3ccc(-c4ccc(C5CCCCC5)cc4)cc3)c3ccc4ccccc4c3-c3cccc4ccccc34)cc2-c2c(C3CCCCC3)cccc21]

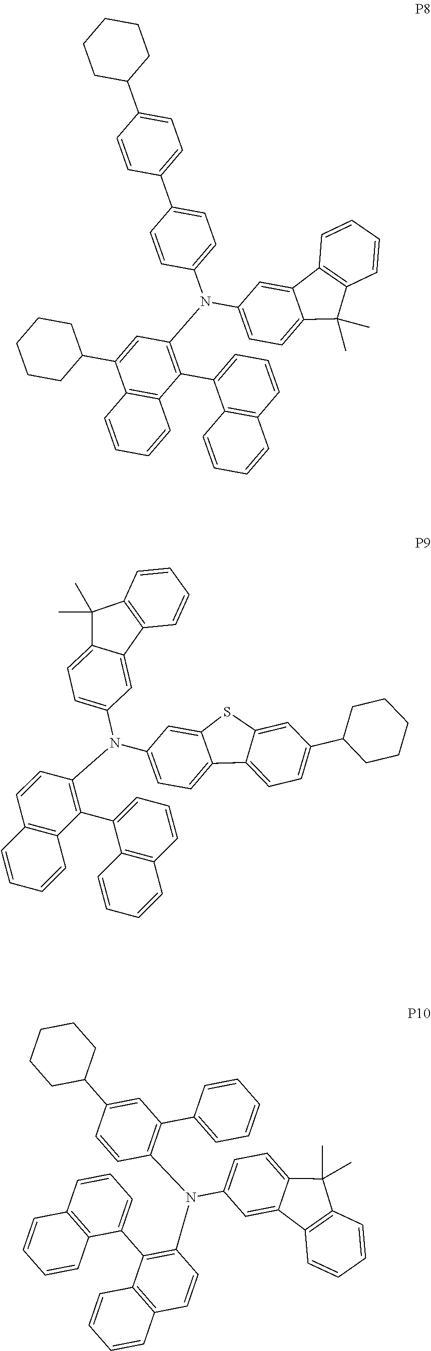 [SMILES: CC1(C)c2ccccc2-c2cc(N(c3ccc(-c4ccc(C5CCCCC5)cc4)cc3)c3cc(C4CCCCC4)c4ccccc4c3-c3cccc4ccccc34)ccc21 ; CC1(C)c2ccccc2-c2cc(N(c3ccc4c(c3)sc3cc(C5CCCCC5)ccc34)c3ccc4ccccc4c3-c3cccc4ccccc34)ccc21 ; CC1(C)c2ccccc2-c2cc(N(c3ccc(C4CCCCC4)cc3-c3ccccc3)c3ccc4ccccc4c3-c3cccc4ccccc34)ccc21]

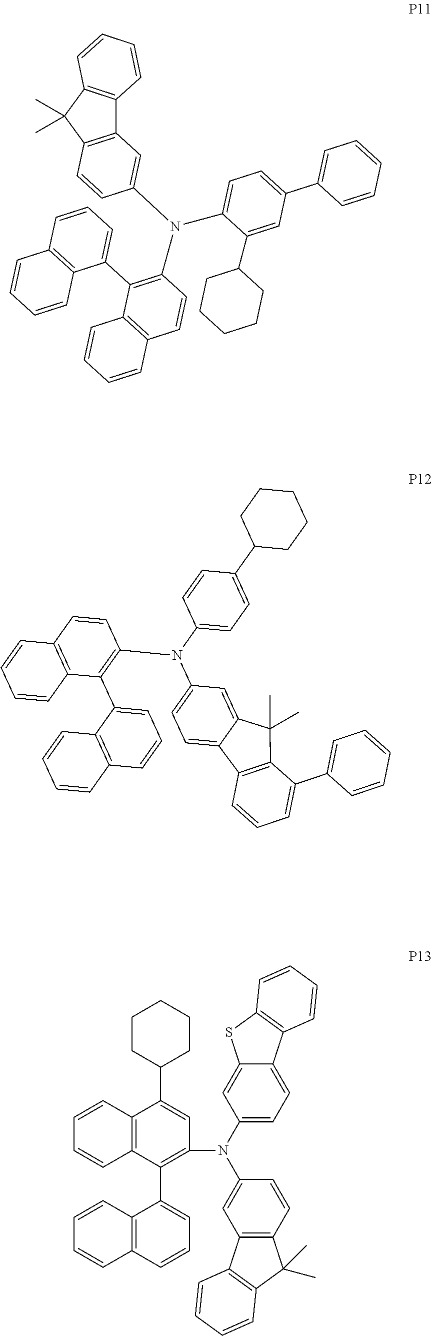 [SMILES: CC1(C)c2ccccc2-c2cc(N(c3ccc(-c4ccccc4)cc3C3CCCCC3)c3ccc4ccccc4c3-c3cccc4ccccc34)ccc21 ; CC1(C)c2cc(N(c3ccc(C4CCCCC4)cc3)c3ccc4ccccc4c3-c3cccc4ccccc34)ccc2-c2cccc(-c3ccccc3)c21 ; CC1(C)c2ccccc2-c2cc(N(c3ccc4c(c3)sc3ccccc34)c3cc(C4CCCCC4)c4ccccc4c3-c3cccc4ccccc34)ccc21]

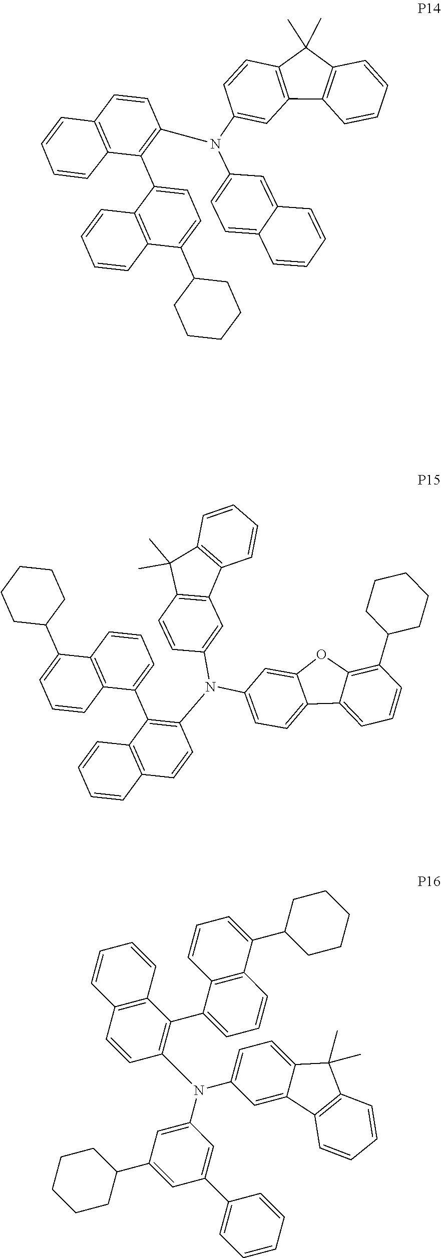 [SMILES: CC1(C)c2ccccc2-c2cc(N(c3ccc4ccccc4c3)c3ccc4ccccc4c3-c3ccc(C4CCCCC4)c4ccccc34)ccc21 ; CC1(C)c2ccccc2-c2cc(N(c3ccc4c(c3)oc3c(C5CCCCC5)cccc34)c3ccc4ccccc4c3-c3cccc4c(C5CCCCC5)cccc34)ccc21 ; CC1(C)c2ccccc2-c2cc(N(c3cc(-c4ccccc4)cc(C4CCCCC4)c3)c3ccc4ccccc4c3-c3cccc4c(C5CCCCC5)cccc34)ccc21]

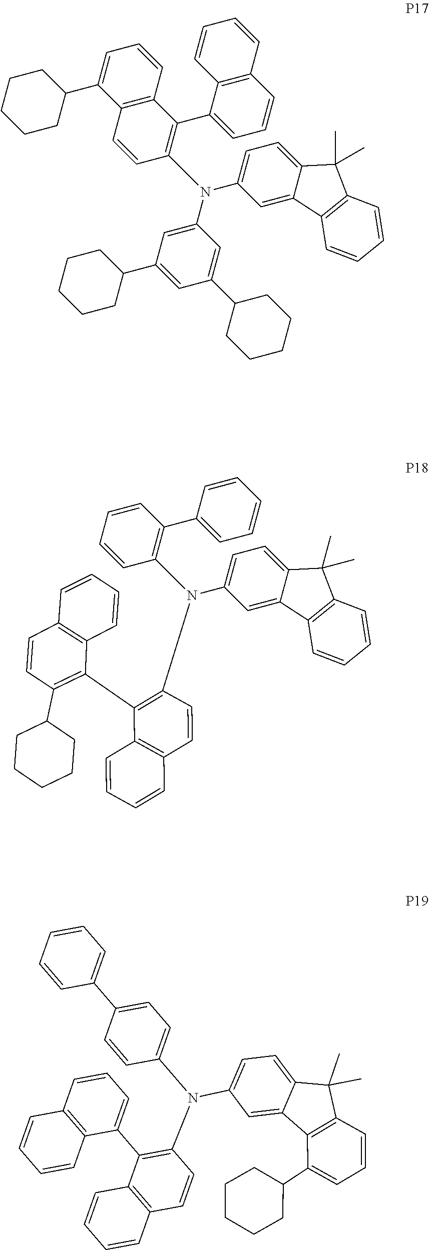 [SMILES: CC1(C)c2ccccc2-c2cc(N(c3cc(C4CCCCC4)cc(C4CCCCC4)c3)c3ccc4c(C5CCCCC5)cccc4c3-c3cccc4ccccc34)ccc21 ; CC1(C)c2ccccc2-c2cc(N(c3ccccc3-c3ccccc3)c3ccc4ccccc4c3-c3c(C4CCCCC4)ccc4ccccc34)ccc21 ; CC1(C)c2ccc(N(c3ccc(-c4ccccc4)cc3)c3ccc4ccccc4c3-c3cccc4ccccc34)cc2-c2c(C3CCCCC3)cccc21]

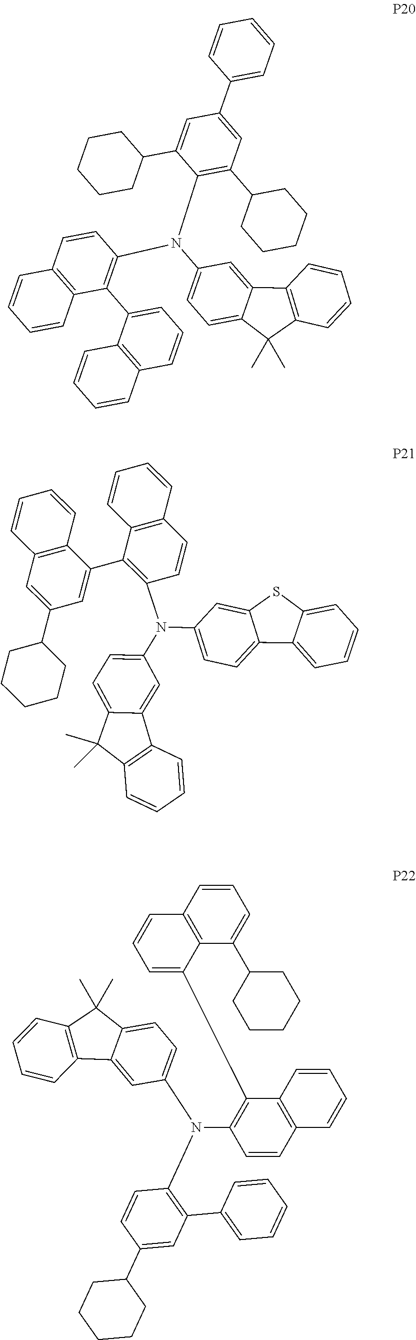 [SMILES: CC1(C)c2ccccc2-c2cc(N(c3ccc4ccccc4c3-c3cccc4ccccc34)c3c(C4CCCCC4)cc(-c4ccccc4)cc3C3CCCCC3)ccc21 ; CC1(C)c2ccccc2-c2cc(N(c3ccc4c(c3)sc3ccccc34)c3ccc4ccccc4c3-c3cc(C4CCCCC4)cc4ccccc34)ccc21 ; CC1(C)c2ccccc2-c2cc(N(c3ccc(C4CCCCC4)cc3-c3ccccc3)c3ccc4ccccc4c3-c3cccc4cccc(C5CCCCC5)c34)ccc21]

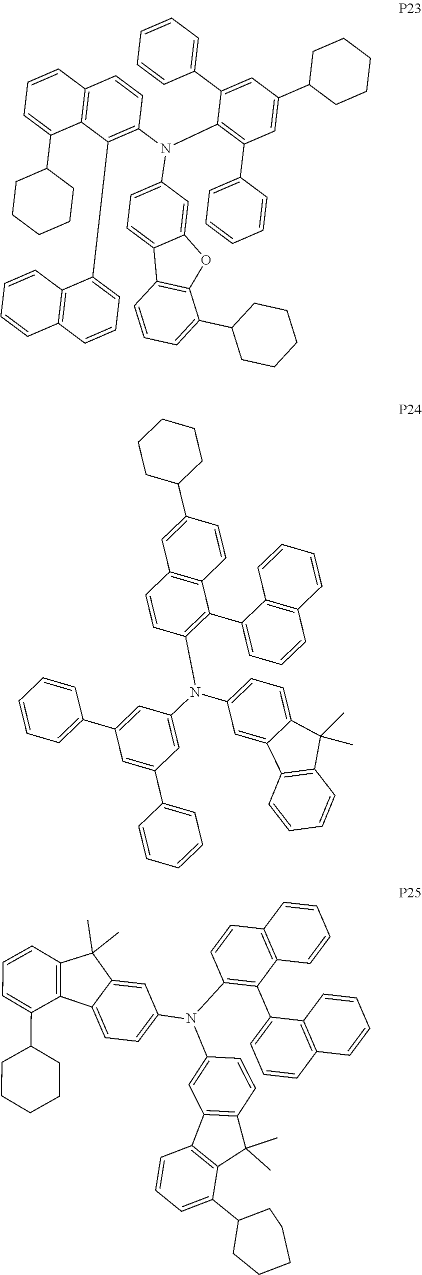 [SMILES: c1ccc(-c2cc(C3CCCCC3)cc(-c3ccccc3)c2N(c2ccc3c(c2)oc2c(C4CCCCC4)cccc23)c2ccc3cccc(C4CCCCC4)c3c2-c2cccc3ccccc23)cc1 ; CC1(C)c2ccccc2-c2cc(N(c3cc(-c4ccccc4)cc(-c4ccccc4)c3)c3ccc4cc(C5CCCCC5)ccc4c3-c3cccc4ccccc34)ccc21 ; CC1(C)c2cc(N(c3ccc4c(c3)-c3cccc(C5CCCCC5)c3C4(C)C)c3ccc4ccccc4c3-c3cccc4ccccc34)ccc2-c2c(C3CCCCC3)cccc21]

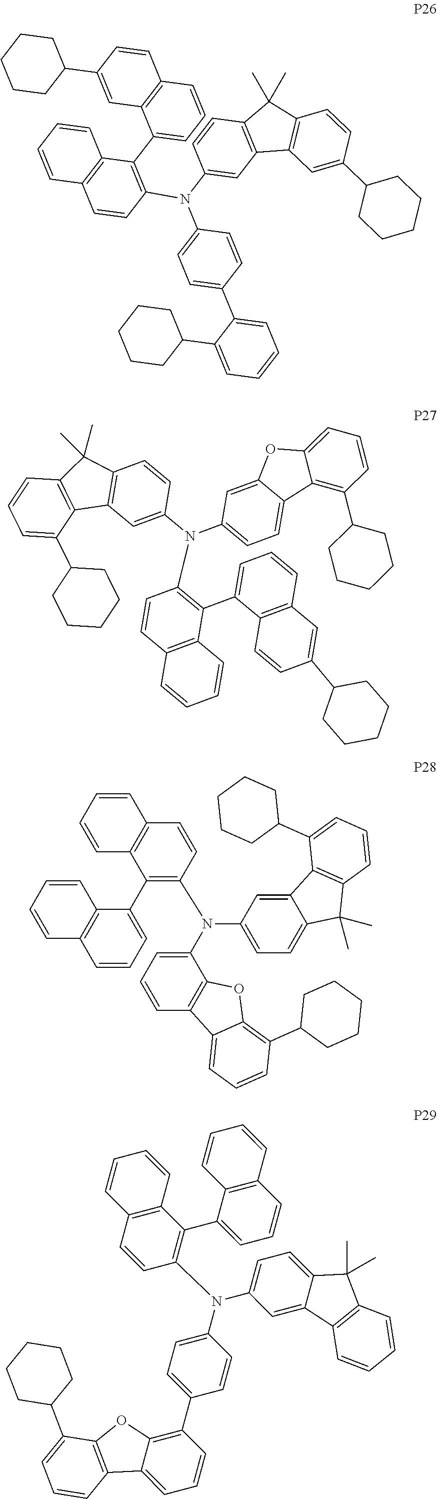

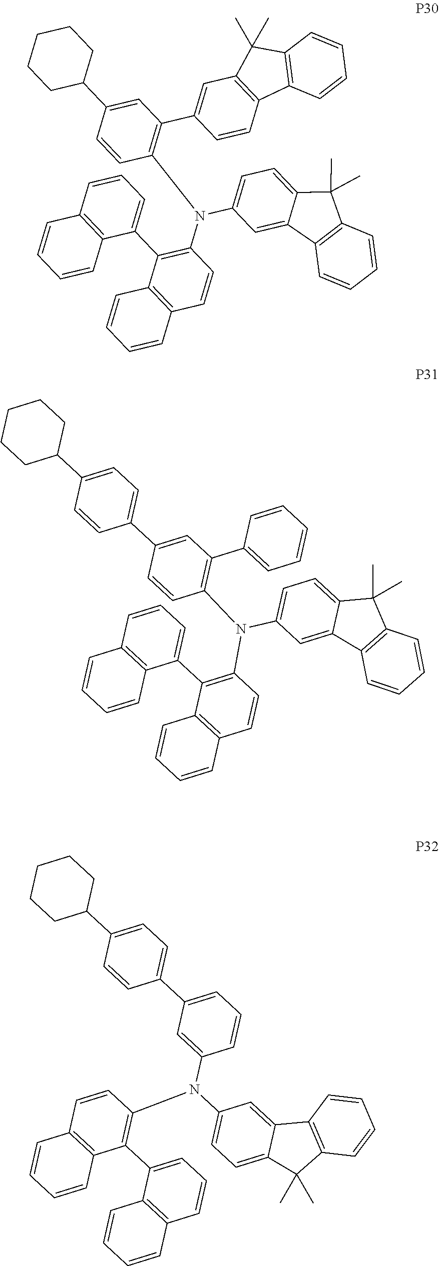 [SMILES: CC1(C)c2ccccc2-c2cc(N(c3ccc(C4CCCCC4)cc3-c3ccc4c(c3)C(C)(C)c3ccccc3-4)c3ccc4ccccc4c3-c3cccc4ccccc34)ccc21 ; CC1(C)c2ccccc2-c2cc(N(c3ccc(-c4ccc(C5CCCCC5)cc4)cc3-c3ccccc3)c3ccc4ccccc4c3-c3cccc4ccccc34)ccc21 ; CC1(C)c2ccccc2-c2cc(N(c3cccc(-c4ccc(C5CCCCC5)cc4)c3)c3ccc4ccccc4c3-c3cccc4ccccc34)ccc21]

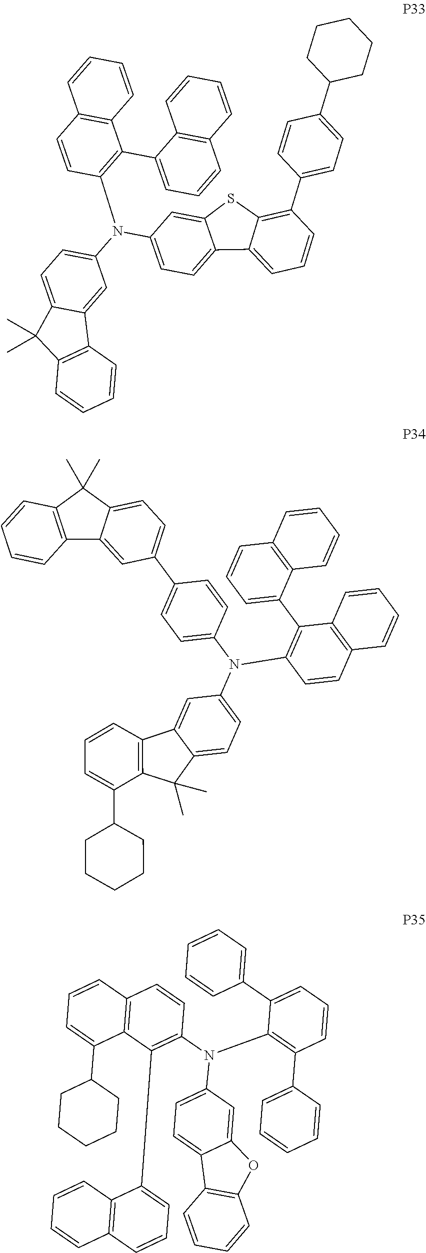 [SMILES: CC1(C)c2ccccc2-c2cc(N(c3ccc4c(c3)sc3c(-c5ccc(C6CCCCC6)cc5)cccc34)c3ccc4ccccc4c3-c3cccc4ccccc34)ccc21 ; CC1(C)c2ccccc2-c2cc(-c3ccc(N(c4ccc5c(c4)-c4cccc(C6CCCCC6)c4C5(C)C)c4ccc5ccccc5c4-c4cccc5ccccc45)cc3)ccc21 ; c1ccc(-c2cccc(-c3ccccc3)c2N(c2ccc3c(c2)oc2ccccc23)c2ccc3cccc(C4CCCCC4)c3c2-c2cccc3ccccc23)cc1]

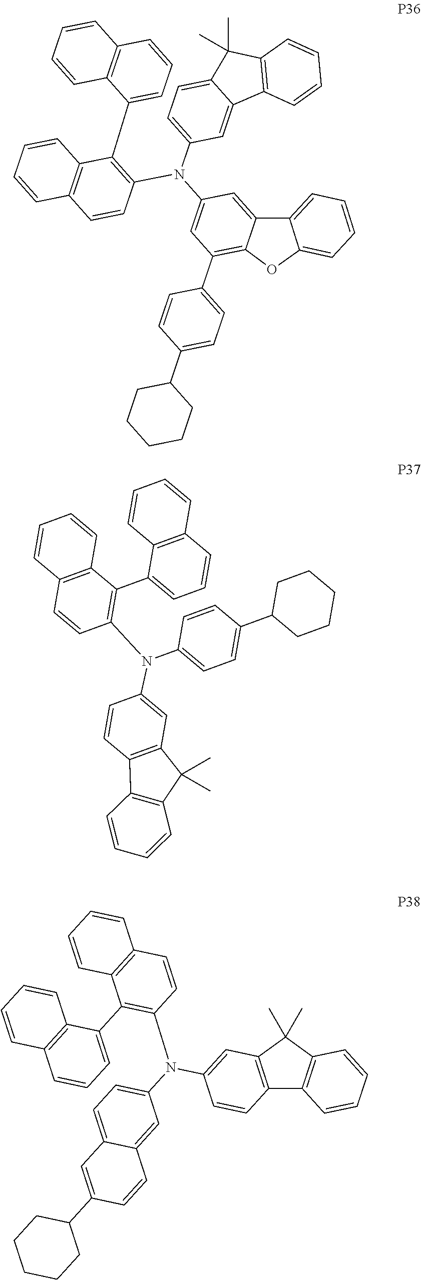

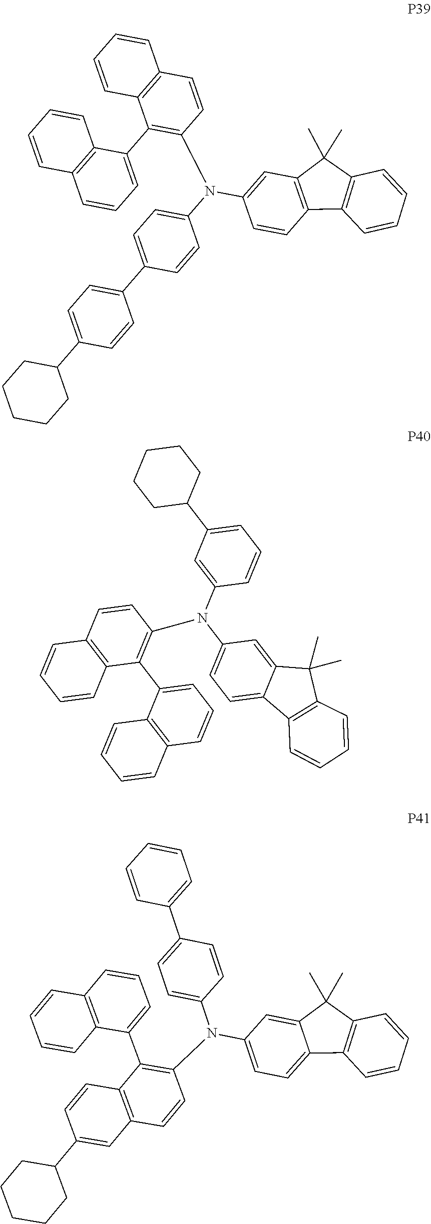 [SMILES: CC1(C)c2ccccc2-c2ccc(N(c3ccc(-c4ccc(C5CCCCC5)cc4)cc3)c3ccc4ccccc4c3-c3cccc4ccccc34)cc21 ; CC1(C)c2ccccc2-c2ccc(N(c3cccc(C4CCCCC4)c3)c3ccc4ccccc4c3-c3cccc4ccccc34)cc21 ; CC1(C)c2ccccc2-c2ccc(N(c3ccc(-c4ccccc4)cc3)c3ccc4cc(C5CCCCC5)ccc4c3-c3cccc4ccccc34)cc21]

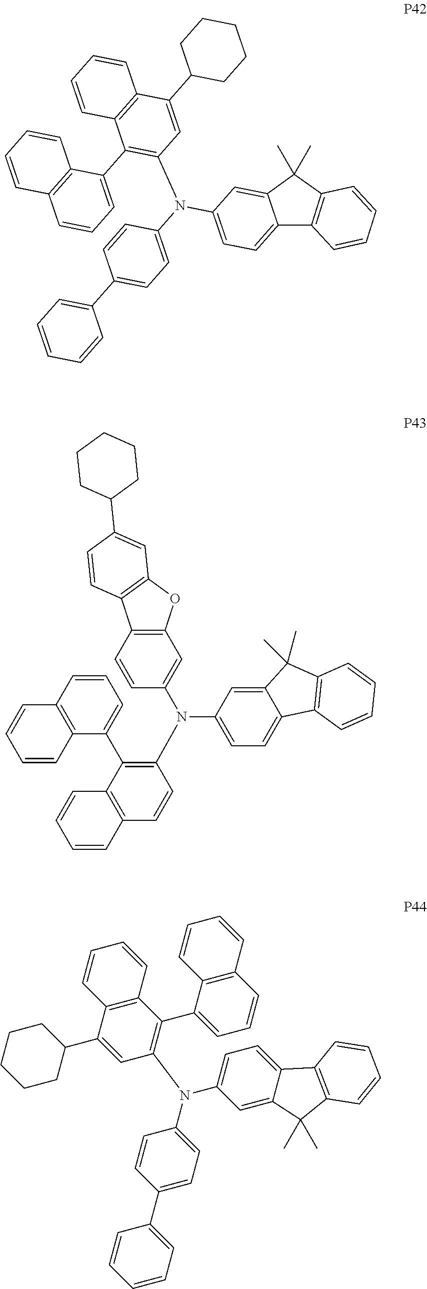 [SMILES: CC1(C)c2ccccc2-c2ccc(N(c3ccc(-c4ccccc4)cc3)c3cc(C4CCCCC4)c4ccccc4c3-c3cccc4ccccc34)cc21 ; CC1(C)c2ccccc2-c2ccc(N(c3ccc4c(c3)oc3cc(C5CCCCC5)ccc34)c3ccc4ccccc4c3-c3cccc4ccccc34)cc21 ; CC1(C)c2ccccc2-c2ccc(N(c3ccc(-c4ccccc4)cc3)c3cc(C4CCCCC4)c4ccccc4c3-c3cccc4ccccc34)cc21]

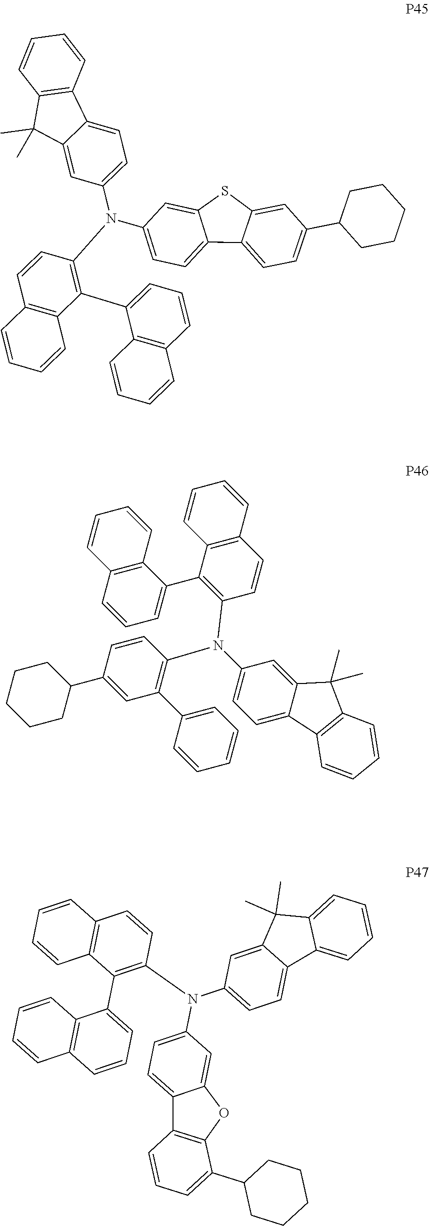 [SMILES: CC1(C)c2ccccc2-c2ccc(N(c3ccc4c(c3)sc3cc(C5CCCCC5)ccc34)c3ccc4ccccc4c3-c3cccc4ccccc34)cc21 ; CC1(C)c2ccccc2-c2ccc(N(c3ccc(C4CCCCC4)cc3-c3ccccc3)c3ccc4ccccc4c3-c3cccc4ccccc34)cc21 ; CC1(C)c2ccccc2-c2ccc(N(c3ccc4c(c3)oc3c(C5CCCCC5)cccc34)c3ccc4ccccc4c3-c3cccc4ccccc34)cc21]

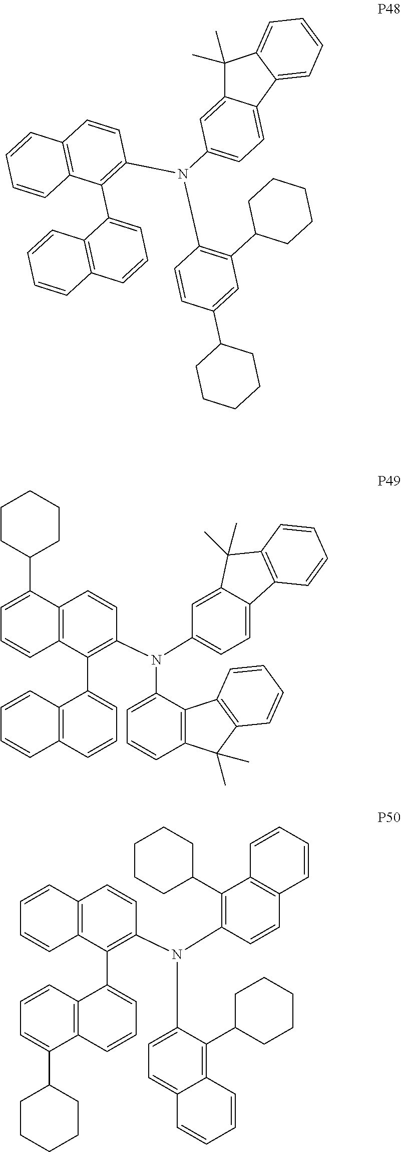 [SMILES: CC1(C)c2ccccc2-c2ccc(N(c3ccc(C4CCCCC4)cc3C3CCCCC3)c3ccc4ccccc4c3-c3cccc4ccccc34)cc21 ; CC1(C)c2ccccc2-c2ccc(N(c3cccc4c3-c3ccccc3C4(C)C)c3ccc4c(C5CCCCC5)cccc4c3-c3cccc4ccccc34)cc21 ; c1ccc2c(-c3cccc4c(C5CCCCC5)cccc34)c(N(c3ccc4ccccc4c3C3CCCCC3)c3ccc4ccccc4c3C3CCCCC3)ccc2c1]

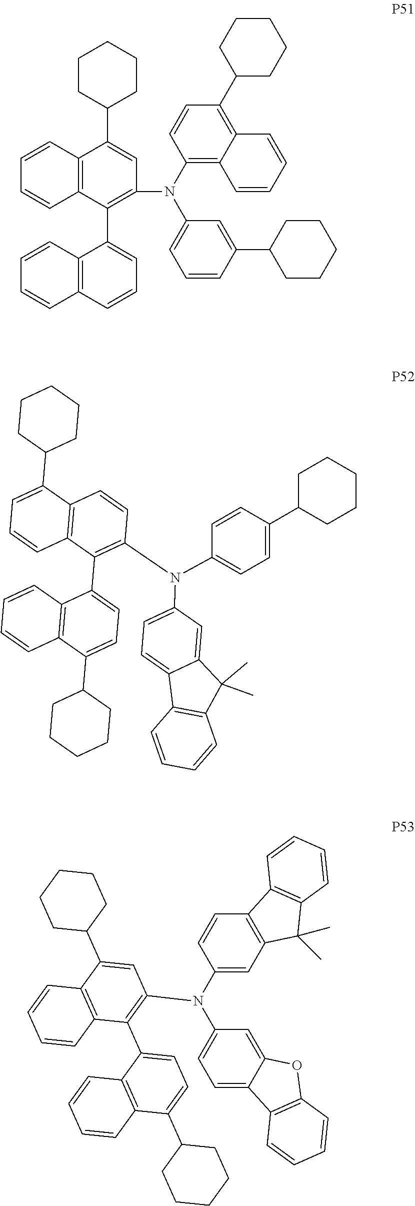 [SMILES: c1cc(C2CCCCC2)cc(N(c2cc(C3CCCCC3)c3ccccc3c2-c2cccc3ccccc23)c2ccc(C3CCCCC3)c3ccccc23)c1 ; CC1(C)c2ccccc2-c2ccc(N(c3ccc(C4CCCCC4)cc3)c3ccc4c(C5CCCCC5)cccc4c3-c3ccc(C4CCCCC4)c4ccccc34)cc21 ; CC1(C)c2ccccc2-c2ccc(N(c3ccc4c(c3)oc3ccccc34)c3cc(C4CCCCC4)c4ccccc4c3-c3ccc(C4CCCCC4)c4ccccc34)cc21]

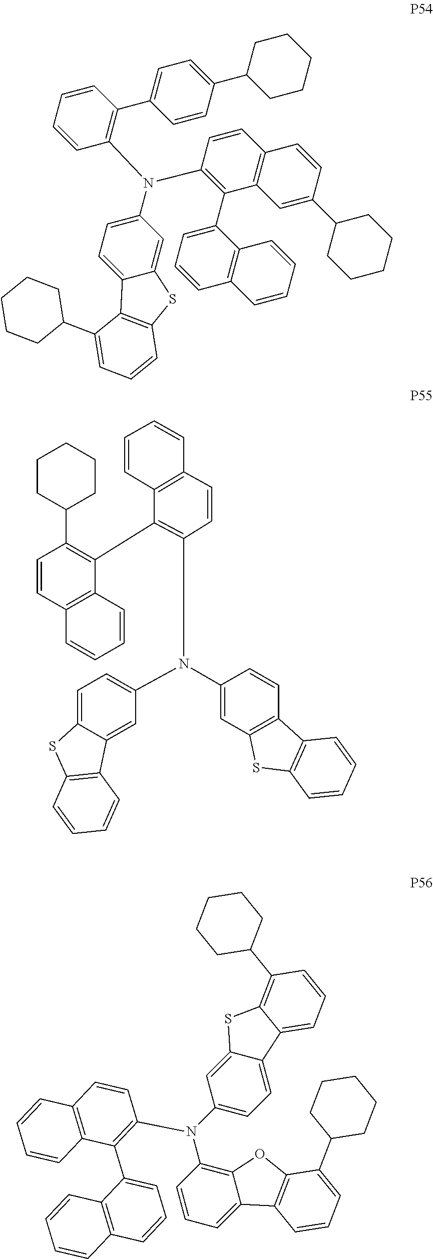 [SMILES: c1ccc(N(c2ccc3c(c2)sc2cccc(C4CCCCC4)c23)c2ccc3ccc(C4CCCCC4)cc3c2-c2cccc3ccccc23)c(-c2ccc(C3CCCCC3)cc2)c1 ; c1ccc2c(-c3c(N(c4ccc5c(c4)sc4ccccc45)c4ccc5sc6ccccc6c5c4)ccc4ccccc34)c(C3CCCCC3)ccc2c1 ; c1ccc2c(-c3c(N(c4ccc5c(c4)sc4c(C6CCCCC6)cccc45)c4cccc5c4oc4c(C6CCCCC6)cccc45)ccc4ccccc34)cccc2c1]

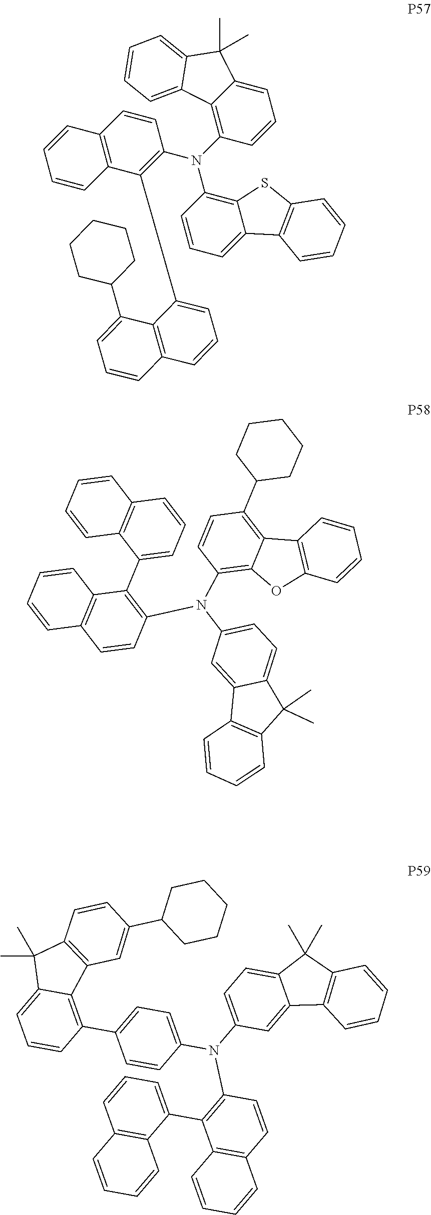 [SMILES: CC1(C)c2ccccc2-c2c(N(c3ccc4ccccc4c3-c3cccc4cccc(C5CCCCC5)c34)c3cccc4c3sc3ccccc34)cccc21 ; CC1(C)c2ccccc2-c2cc(N(c3ccc4ccccc4c3-c3cccc4ccccc34)c3ccc(C4CCCCC4)c4c3oc3ccccc34)ccc21 ; CC1(C)c2ccccc2-c2cc(N(c3ccc(-c4cccc5c4-c4cc(C6CCCCC6)ccc4C5(C)C)cc3)c3ccc4ccccc4c3-c3cccc4ccccc34)ccc21]

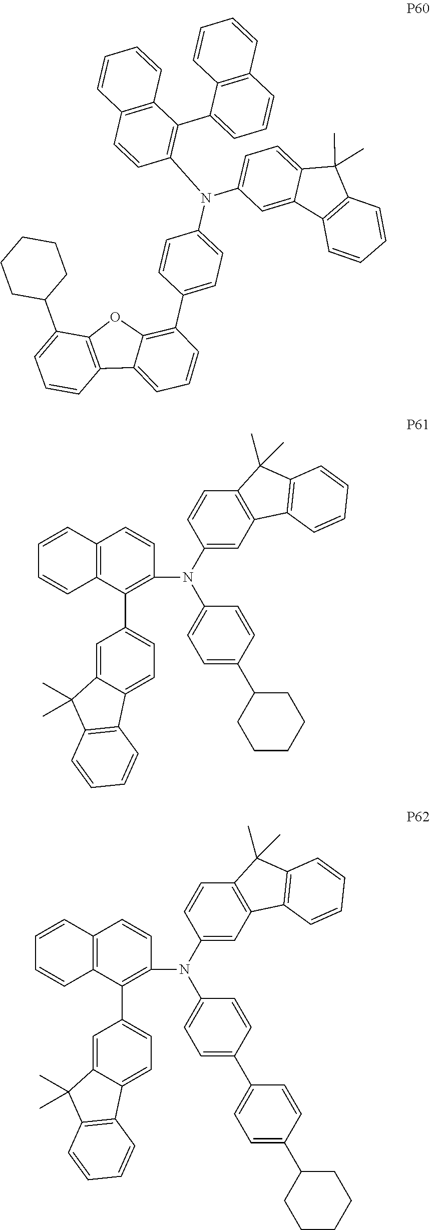 [SMILES: CC1(C)c2ccccc2-c2cc(N(c3ccc(-c4cccc5c4oc4c(C6CCCCC6)cccc45)cc3)c3ccc4ccccc4c3-c3cccc4ccccc34)ccc21 ; CC1(C)c2ccccc2-c2cc(N(c3ccc(C4CCCCC4)cc3)c3ccc4ccccc4c3-c3ccc4c(c3)C(C)(C)c3ccccc3-4)ccc21 ; CC1(C)c2ccccc2-c2cc(N(c3ccc(-c4ccc(C5CCCCC5)cc4)cc3)c3ccc4ccccc4c3-c3ccc4c(c3)C(C)(C)c3ccccc3-4)ccc21]

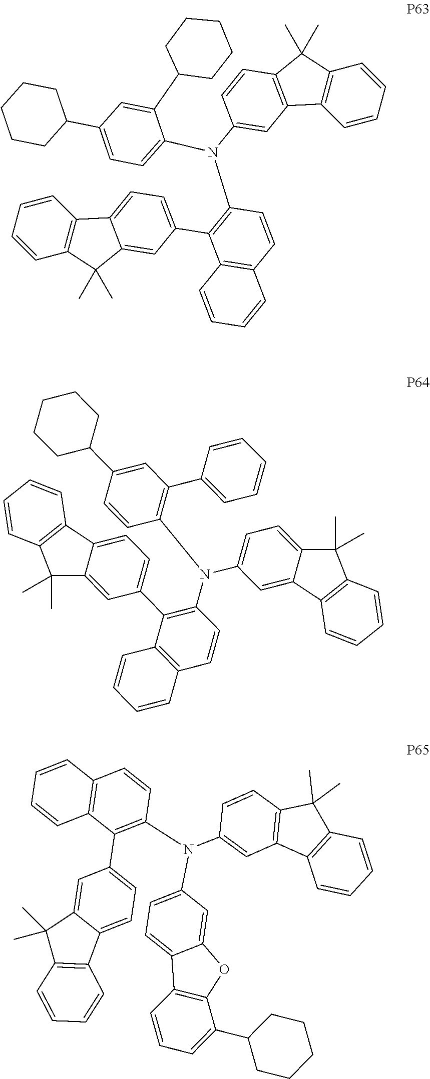 [SMILES: CC1(C)c2ccccc2-c2cc(N(c3ccc(C4CCCCC4)cc3C3CCCCC3)c3ccc4ccccc4c3-c3ccc4c(c3)C(C)(C)c3ccccc3-4)ccc21 ; CC1(C)c2ccccc2-c2cc(N(c3ccc(C4CCCCC4)cc3-c3ccccc3)c3ccc4ccccc4c3-c3ccc4c(c3)C(C)(C)c3ccccc3-4)ccc21 ; CC1(C)c2ccccc2-c2cc(N(c3ccc4c(c3)oc3c(C5CCCCC5)cccc34)c3ccc4ccccc4c3-c3ccc4c(c3)C(C)(C)c3ccccc3-4)ccc21]

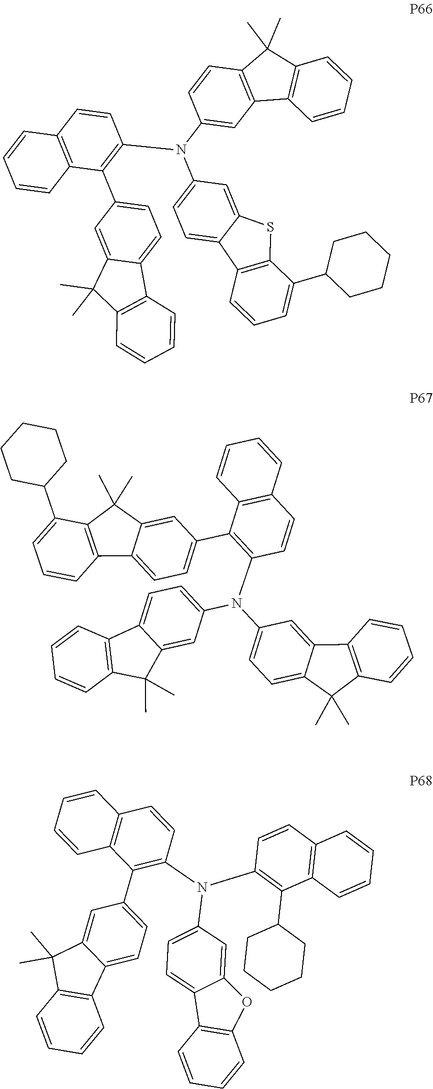 [SMILES: CC1(C)c2ccccc2-c2cc(N(c3ccc4c(c3)sc3c(C5CCCCC5)cccc34)c3ccc4ccccc4c3-c3ccc4c(c3)C(C)(C)c3ccccc3-4)ccc21 ; CC1(C)c2ccccc2-c2cc(N(c3ccc4c(c3)C(C)(C)c3ccccc3-4)c3ccc4ccccc4c3-c3ccc4c(c3)C(C)(C)c3c-4cccc3C3CCCCC3)ccc21 ; CC1(C)c2ccccc2-c2ccc(-c3c(N(c4ccc5c(c4)oc4ccccc45)c4ccc5ccccc5c4C4CCCCC4)ccc4ccccc34)cc21]

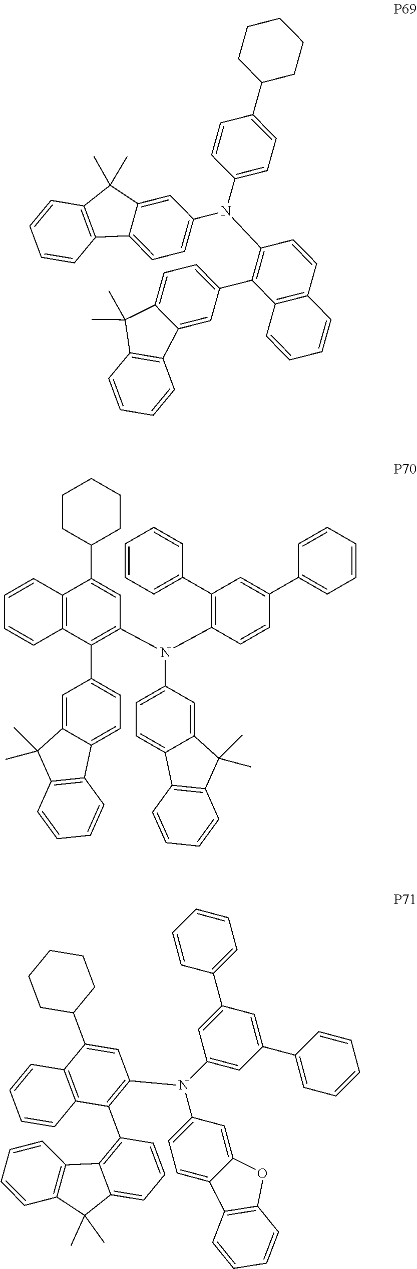 [SMILES: CC1(C)c2ccccc2-c2cc(-c3c(N(c4ccc(C5CCCCC5)cc4)c4ccc5c(c4)C(C)(C)c4ccccc4-5)ccc4ccccc34)ccc21 ; CC1(C)c2ccccc2-c2ccc(-c3c(N(c4ccc5c(c4)C(C)(C)c4ccccc4-5)c4ccc(-c5ccccc5)cc4-c4ccccc4)cc(C4CCCCC4)c4ccccc34)cc21 ; CC1(C)c2ccccc2-c2c(-c3c(N(c4cc(-c5ccccc5)cc(-c5ccccc5)c4)c4ccc5c(c4)oc4ccccc45)cc(C4CCCCC4)c4ccccc34)cccc21]

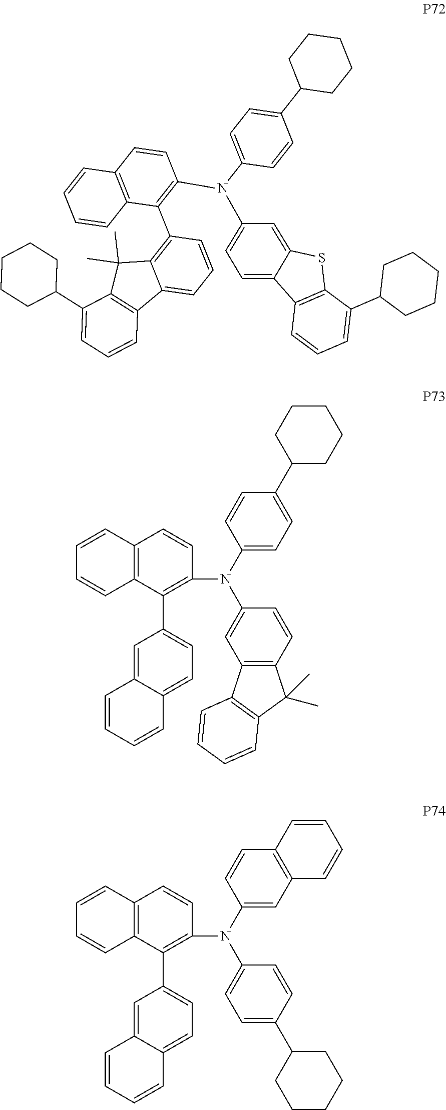 [SMILES: CC1(C)c2c(cccc2-c2c(N(c3ccc(C4CCCCC4)cc3)c3ccc4c(c3)sc3c(C5CCCCC5)cccc34)ccc3ccccc23)-c2cccc(C3CCCCC3)c21 ; CC1(C)c2ccccc2-c2cc(N(c3ccc(C4CCCCC4)cc3)c3ccc4ccccc4c3-c3ccc4ccccc4c3)ccc21 ; c1ccc2cc(-c3c(N(c4ccc(C5CCCCC5)cc4)c4ccc5ccccc5c4)ccc4ccccc34)ccc2c1]

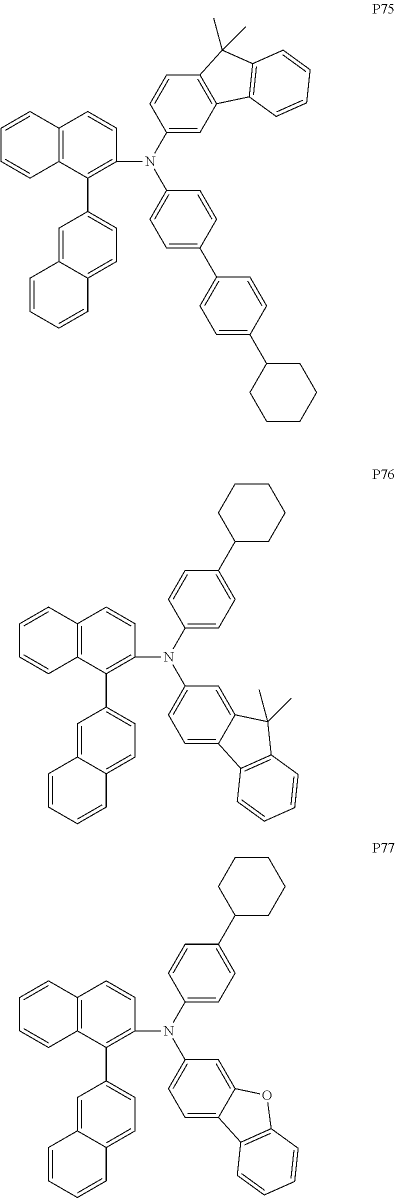

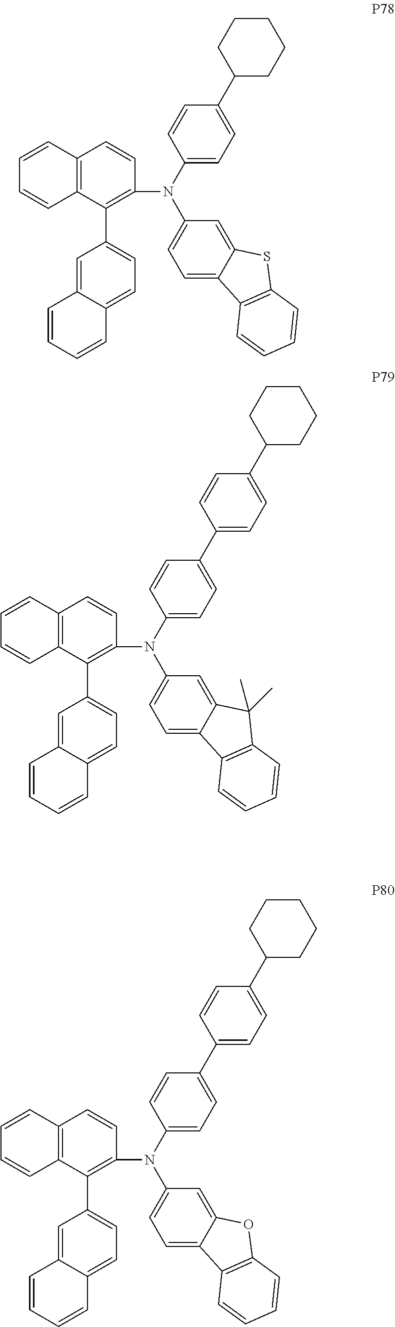

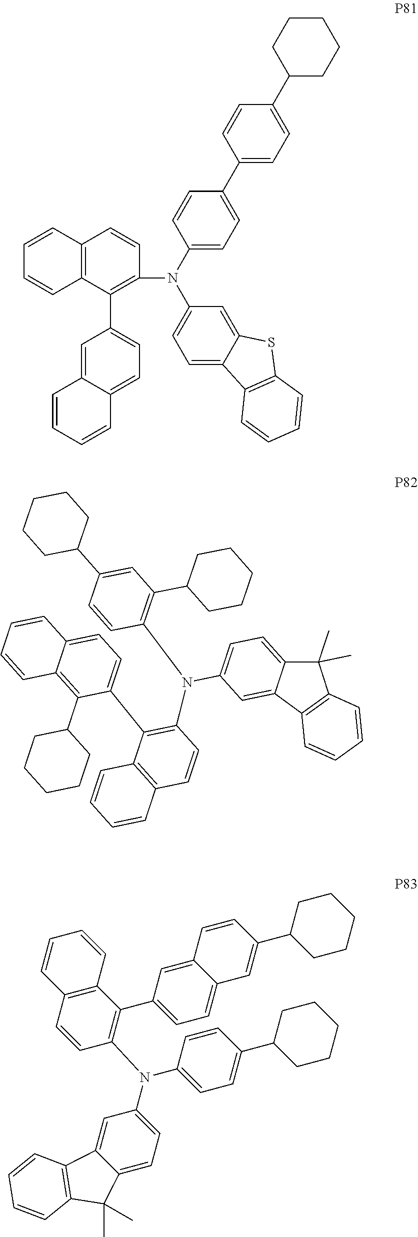 [SMILES: c1ccc2cc(-c3c(N(c4ccc(-c5ccc(C6CCCCC6)cc5)cc4)c4ccc5c(c4)sc4ccccc45)ccc4ccccc34)ccc2c1 ; CC1(C)c2ccccc2-c2cc(N(c3ccc(C4CCCCC4)cc3C3CCCCC3)c3ccc4ccccc4c3-c3ccc4ccccc4c3C3CCCCC3)ccc21 ; CC1(C)c2ccccc2-c2cc(N(c3ccc(C4CCCCC4)cc3)c3ccc4ccccc4c3-c3ccc4cc(C5CCCCC5)ccc4c3)ccc21]

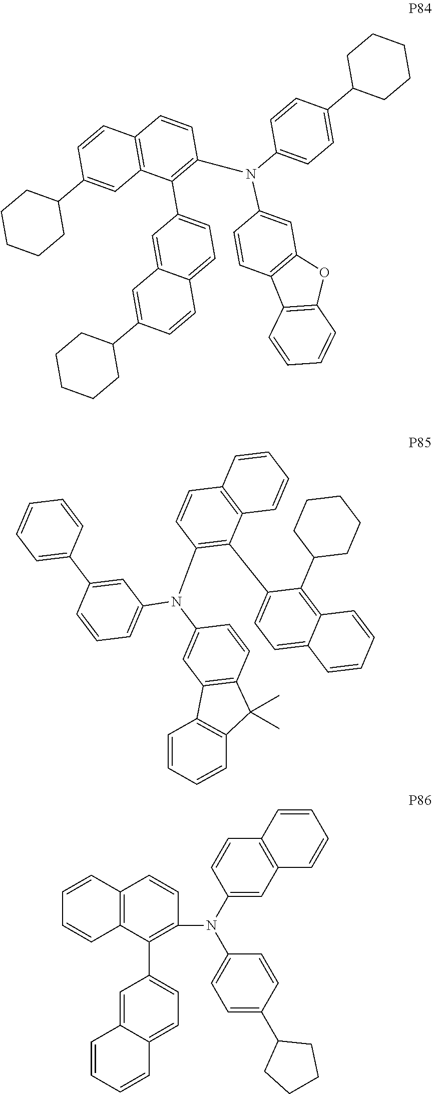 [SMILES: c1ccc2c(c1)oc1cc(N(c3ccc(C4CCCCC4)cc3)c3ccc4ccc(C5CCCCC5)cc4c3-c3ccc4ccc(C5CCCCC5)cc4c3)ccc12 ; CC1(C)c2ccccc2-c2cc(N(c3cccc(-c4ccccc4)c3)c3ccc4ccccc4c3-c3ccc4ccccc4c3C3CCCCC3)ccc21 ; c1ccc2cc(-c3c(N(c4ccc(C5CCCC5)cc4)c4ccc5ccccc5c4)ccc4ccccc34)ccc2c1]

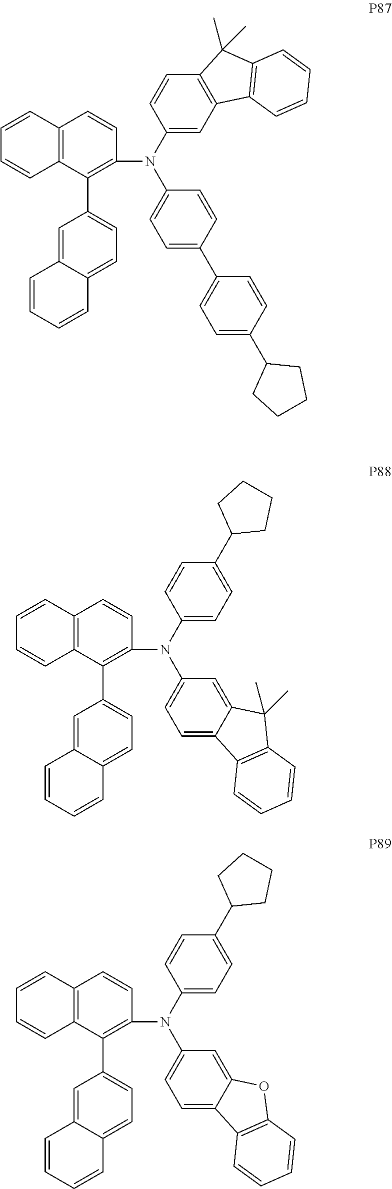

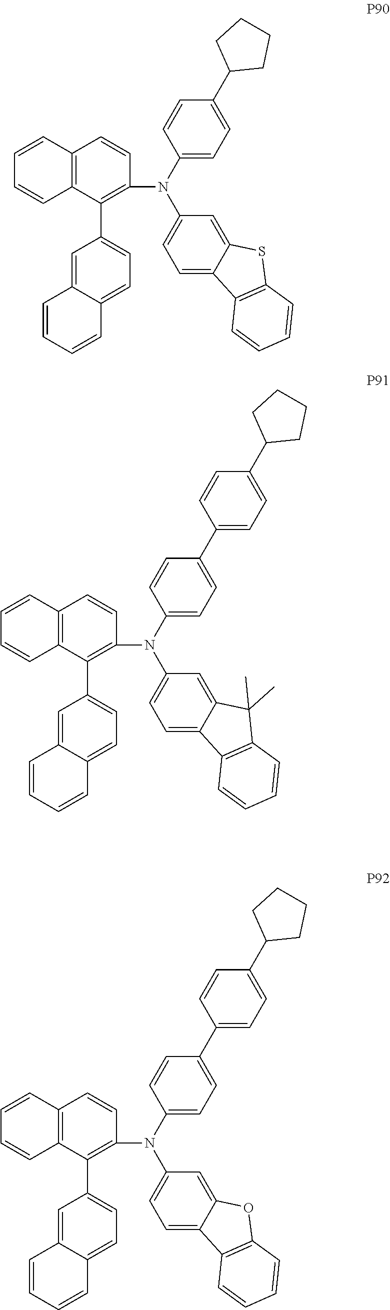

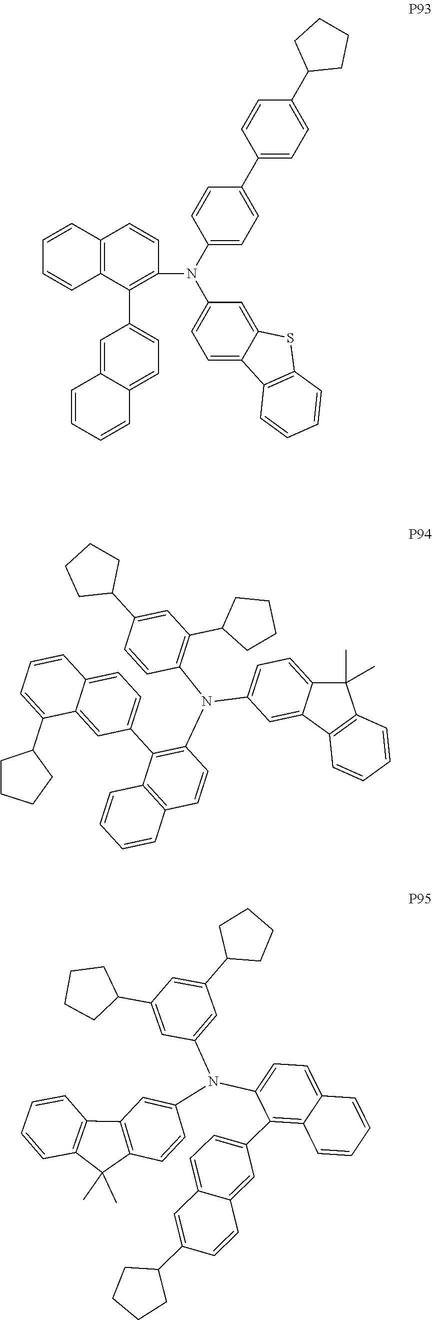 [SMILES: c1ccc2cc(-c3c(N(c4ccc(-c5ccc(C6CCCC6)cc5)cc4)c4ccc5c(c4)sc4ccccc45)ccc4ccccc34)ccc2c1 ; CC1(C)c2ccccc2-c2cc(N(c3ccc(C4CCCC4)cc3C3CCCC3)c3ccc4ccccc4c3-c3ccc4cccc(C5CCCC5)c4c3)ccc21 ; CC1(C)c2ccccc2-c2cc(N(c3cc(C4CCCC4)cc(C4CCCC4)c3)c3ccc4ccccc4c3-c3ccc4cc(C5CCCC5)ccc4c3)ccc21]

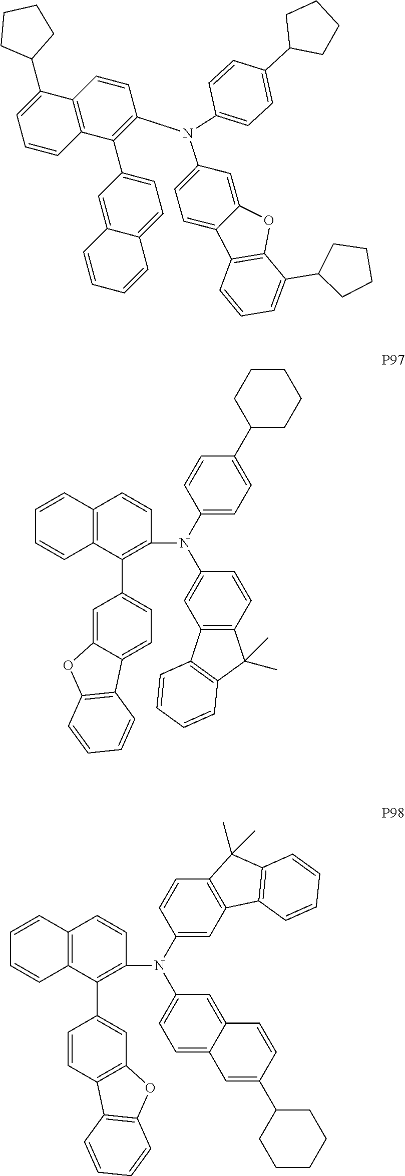 [SMILES: c1ccc2cc(-c3c(N(c4ccc(C5CCCC5)cc4)c4ccc5c(c4)oc4c(C6CCCC6)cccc45)ccc4c(C5CCCC5)cccc34)ccc2c1 ; CC1(C)c2ccccc2-c2cc(N(c3ccc(C4CCCCC4)cc3)c3ccc4ccccc4c3-c3ccc4c(c3)oc3ccccc34)ccc21 ; CC1(C)c2ccccc2-c2cc(N(c3ccc4cc(C5CCCCC5)ccc4c3)c3ccc4ccccc4c3-c3ccc4c(c3)oc3ccccc34)ccc21]

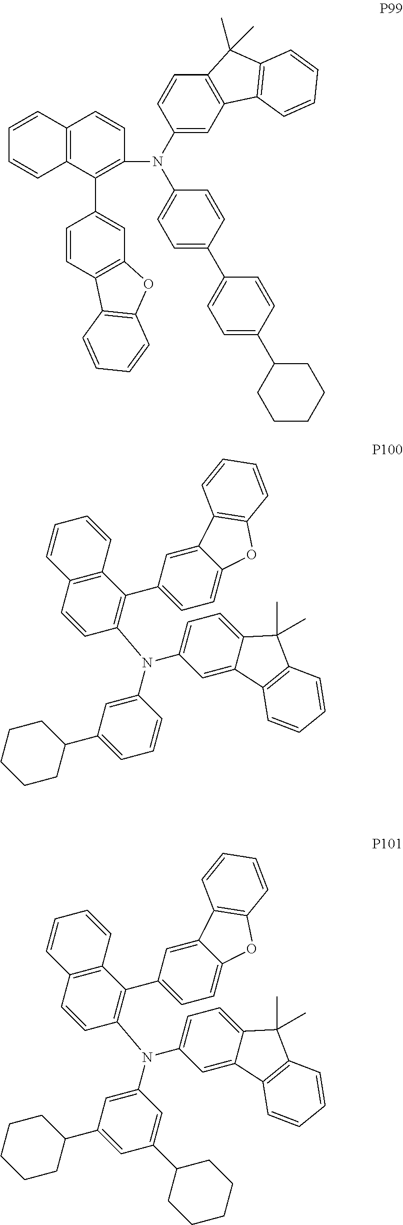 [SMILES: CC1(C)c2ccccc2-c2cc(N(c3ccc(-c4ccc(C5CCCCC5)cc4)cc3)c3ccc4ccccc4c3-c3ccc4c(c3)oc3ccccc34)ccc21 ; CC1(C)c2ccccc2-c2cc(N(c3cccc(C4CCCCC4)c3)c3ccc4ccccc4c3-c3ccc4oc5ccccc5c4c3)ccc21 ; CC1(C)c2ccccc2-c2cc(N(c3cc(C4CCCCC4)cc(C4CCCCC4)c3)c3ccc4ccccc4c3-c3ccc4oc5ccccc5c4c3)ccc21]

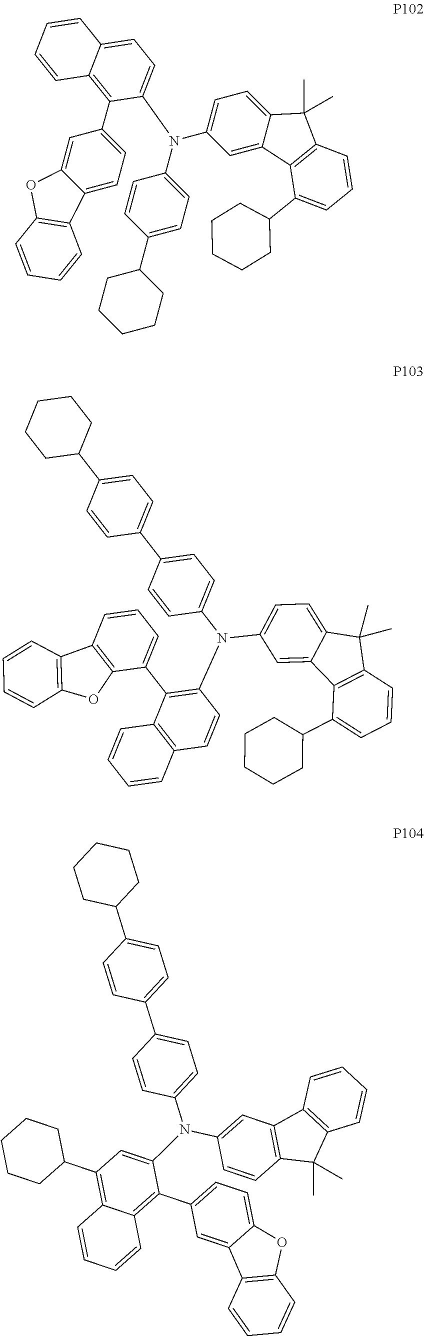 [SMILES: CC1(C)c2ccc(N(c3ccc(C4CCCCC4)cc3)c3ccc4ccccc4c3-c3ccc4c(c3)oc3ccccc34)cc2-c2c(C3CCCCC3)cccc21 ; CC1(C)c2ccc(N(c3ccc(-c4ccc(C5CCCCC5)cc4)cc3)c3ccc4ccccc4c3-c3cccc4c3oc3ccccc34)cc2-c2c(C3CCCCC3)cccc21 ; CC1(C)c2ccccc2-c2cc(N(c3ccc(-c4ccc(C5CCCCC5)cc4)cc3)c3cc(C4CCCCC4)c4ccccc4c3-c3ccc4oc5ccccc5c4c3)ccc21]

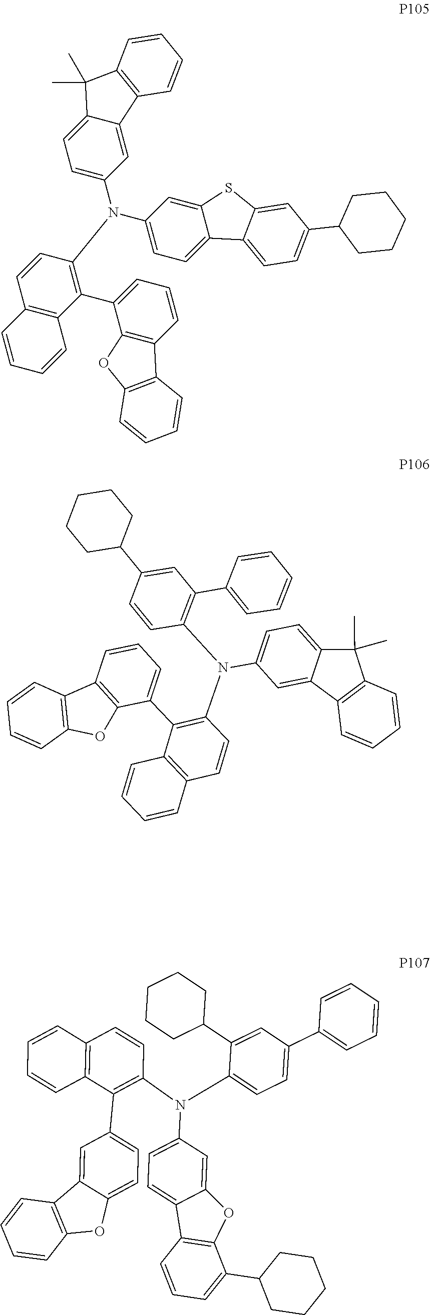 [SMILES: CC1(C)c2ccccc2-c2cc(N(c3ccc4c(c3)sc3cc(C5CCCCC5)ccc34)c3ccc4ccccc4c3-c3cccc4c3oc3ccccc34)ccc21 ; CC1(C)c2ccccc2-c2cc(N(c3ccc(C4CCCCC4)cc3-c3ccccc3)c3ccc4ccccc4c3-c3cccc4c3oc3ccccc34)ccc21 ; c1ccc(-c2ccc(N(c3ccc4c(c3)oc3c(C5CCCCC5)cccc34)c3ccc4ccccc4c3-c3ccc4oc5ccccc5c4c3)c(C3CCCCC3)c2)cc1]

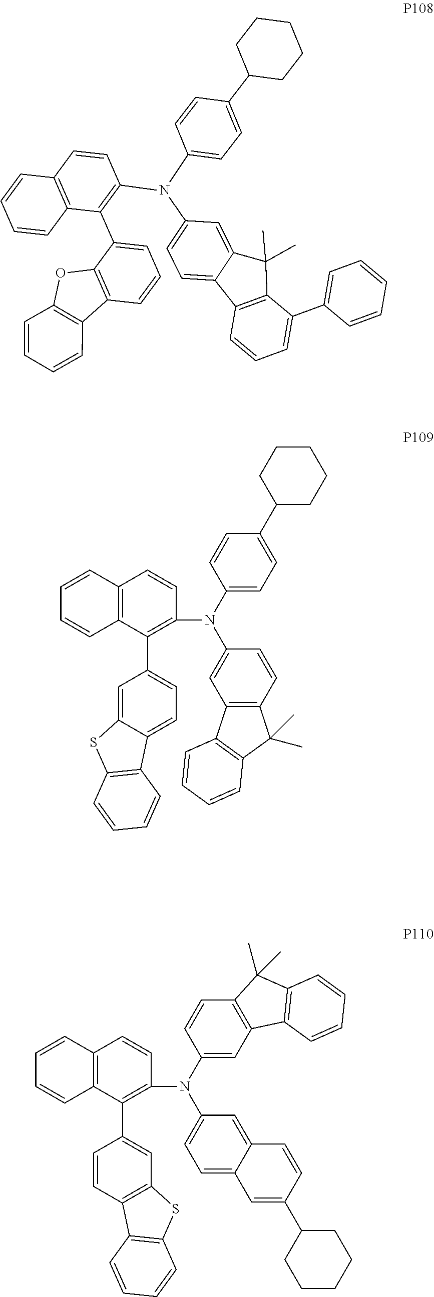 [SMILES: CC1(C)c2cc(N(c3ccc(C4CCCCC4)cc3)c3ccc4ccccc4c3-c3cccc4c3oc3ccccc34)ccc2-c2cccc(-c3ccccc3)c21 ; CC1(C)c2ccccc2-c2cc(N(c3ccc(C4CCCCC4)cc3)c3ccc4ccccc4c3-c3ccc4c(c3)sc3ccccc34)ccc21 ; CC1(C)c2ccccc2-c2cc(N(c3ccc4cc(C5CCCCC5)ccc4c3)c3ccc4ccccc4c3-c3ccc4c(c3)sc3ccccc34)ccc21]

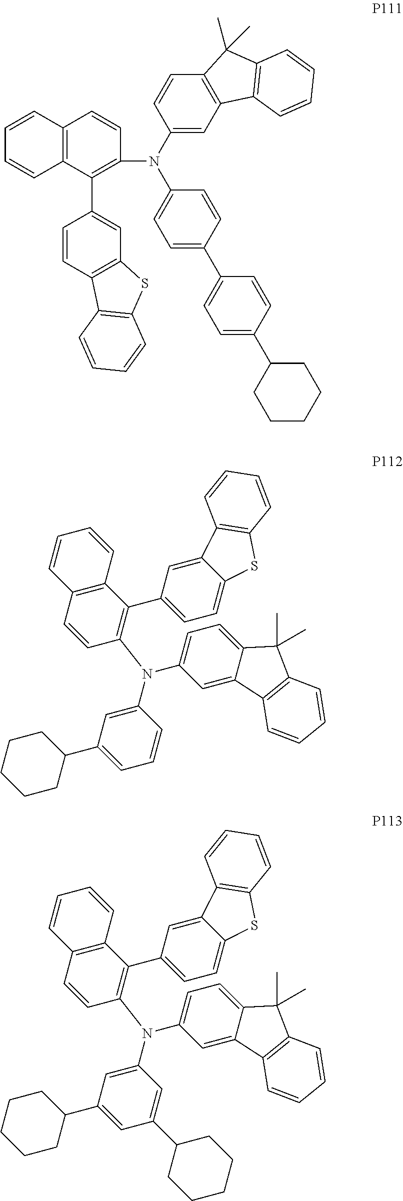 [SMILES: CC1(C)c2ccccc2-c2cc(N(c3ccc(-c4ccc(C5CCCCC5)cc4)cc3)c3ccc4ccccc4c3-c3ccc4c(c3)sc3ccccc34)ccc21 ; CC1(C)c2ccccc2-c2cc(N(c3cccc(C4CCCCC4)c3)c3ccc4ccccc4c3-c3ccc4sc5ccccc5c4c3)ccc21 ; CC1(C)c2ccccc2-c2cc(N(c3cc(C4CCCCC4)cc(C4CCCCC4)c3)c3ccc4ccccc4c3-c3ccc4sc5ccccc5c4c3)ccc21]

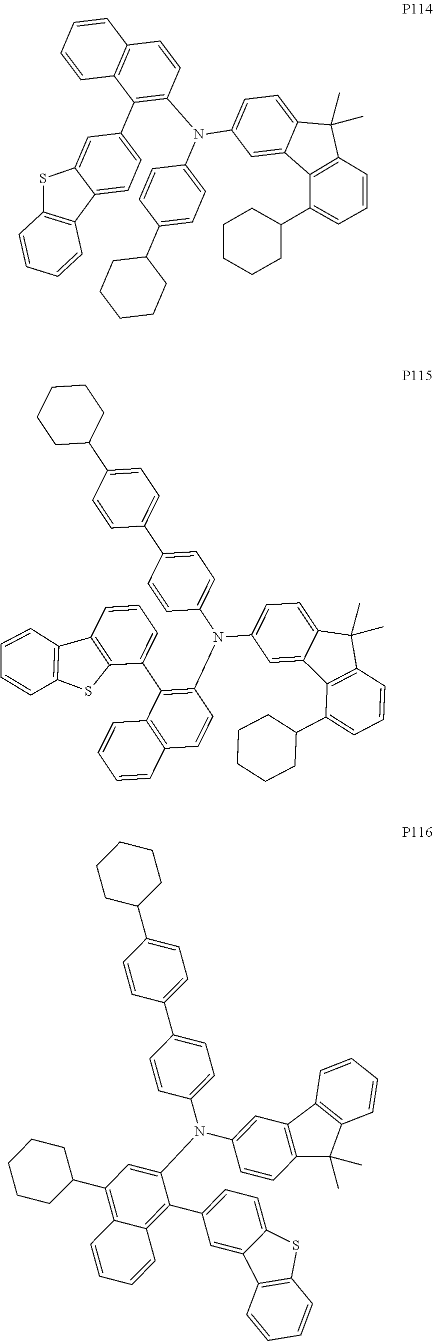 [SMILES: CC1(C)c2ccc(N(c3ccc(C4CCCCC4)cc3)c3ccc4ccccc4c3-c3ccc4c(c3)sc3ccccc34)cc2-c2c(C3CCCCC3)cccc21 ; CC1(C)c2ccc(N(c3ccc(-c4ccc(C5CCCCC5)cc4)cc3)c3ccc4ccccc4c3-c3cccc4c3sc3ccccc34)cc2-c2c(C3CCCCC3)cccc21 ; CC1(C)c2ccccc2-c2cc(N(c3ccc(-c4ccc(C5CCCCC5)cc4)cc3)c3cc(C4CCCCC4)c4ccccc4c3-c3ccc4sc5ccccc5c4c3)ccc21]

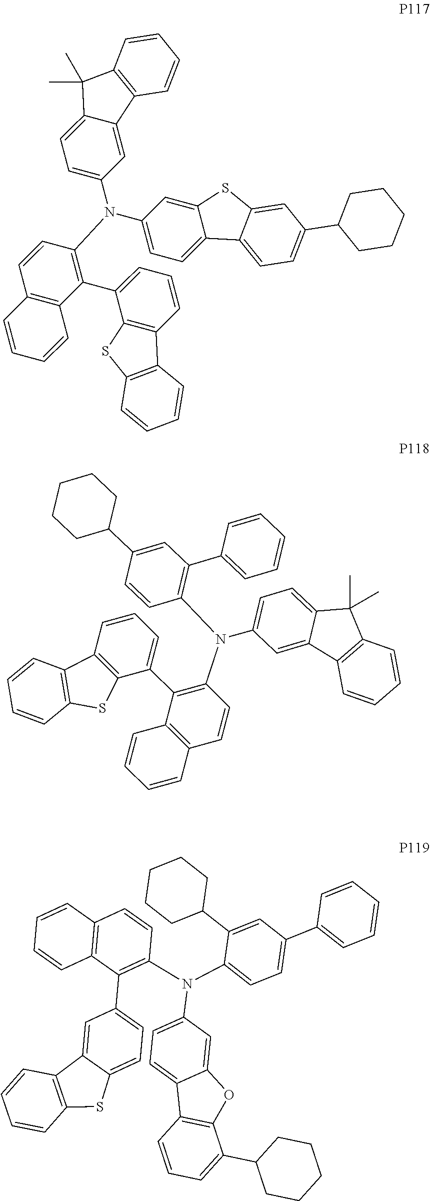 [SMILES: CC1(C)c2ccccc2-c2cc(N(c3ccc4c(c3)sc3cc(C5CCCCC5)ccc34)c3ccc4ccccc4c3-c3cccc4c3sc3ccccc34)ccc21 ; CC1(C)c2ccccc2-c2cc(N(c3ccc(C4CCCCC4)cc3-c3ccccc3)c3ccc4ccccc4c3-c3cccc4c3sc3ccccc34)ccc21 ; c1ccc(-c2ccc(N(c3ccc4c(c3)oc3c(C5CCCCC5)cccc34)c3ccc4ccccc4c3-c3ccc4sc5ccccc5c4c3)c(C3CCCCC3)c2)cc1]

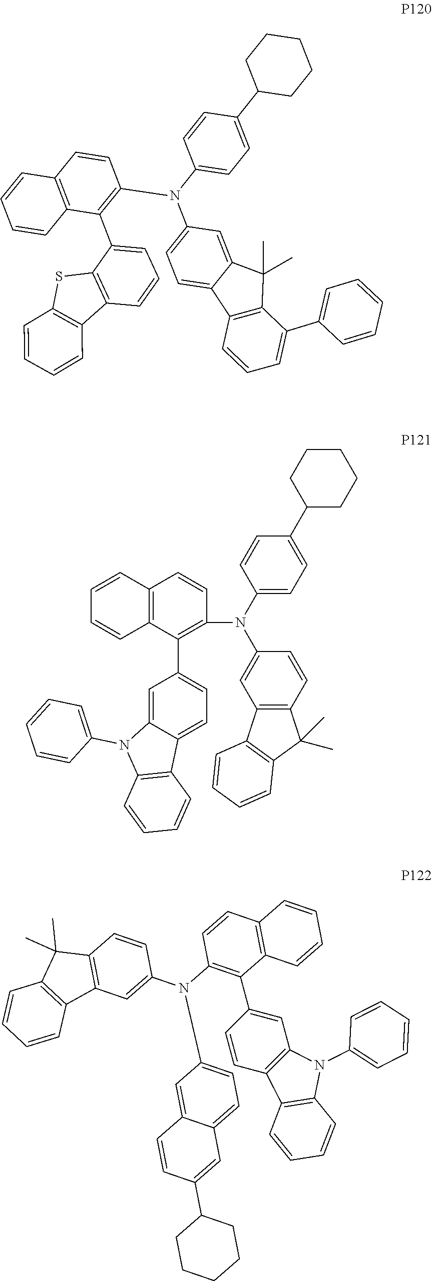 [SMILES: CC1(C)c2cc(N(c3ccc(C4CCCCC4)cc3)c3ccc4ccccc4c3-c3cccc4c3sc3ccccc34)ccc2-c2cccc(-c3ccccc3)c21 ; CC1(C)c2ccccc2-c2cc(N(c3ccc(C4CCCCC4)cc3)c3ccc4ccccc4c3-c3ccc4c5ccccc5n(-c5ccccc5)c4c3)ccc21 ; CC1(C)c2ccccc2-c2cc(N(c3ccc4cc(C5CCCCC5)ccc4c3)c3ccc4ccccc4c3-c3ccc4c5ccccc5n(-c5ccccc5)c4c3)ccc21]

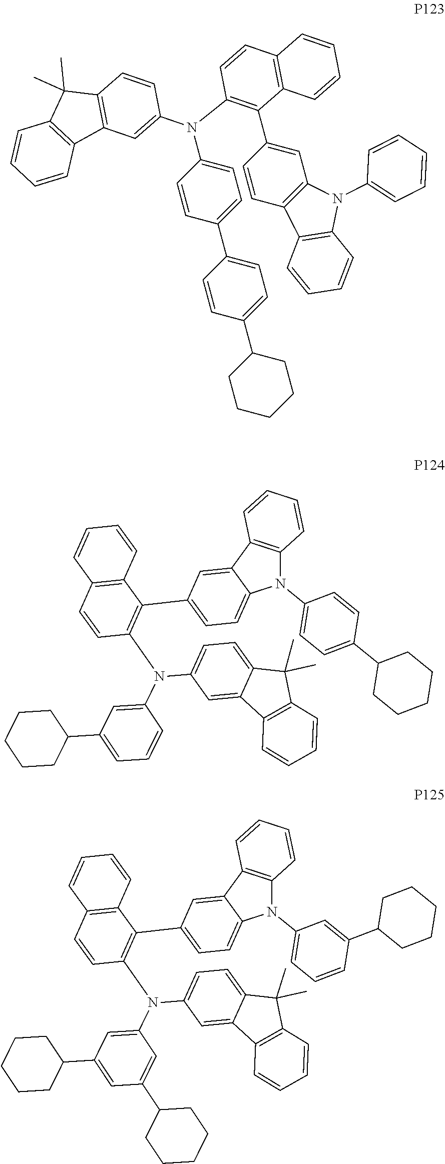 [SMILES: CC1(C)c2ccccc2-c2cc(N(c3ccc(-c4ccc(C5CCCCC5)cc4)cc3)c3ccc4ccccc4c3-c3ccc4c5ccccc5n(-c5ccccc5)c4c3)ccc21 ; CC1(C)c2ccccc2-c2cc(N(c3cccc(C4CCCCC4)c3)c3ccc4ccccc4c3-c3ccc4c(c3)c3ccccc3n4-c3ccc(C4CCCCC4)cc3)ccc21 ; CC1(C)c2ccccc2-c2cc(N(c3cc(C4CCCCC4)cc(C4CCCCC4)c3)c3ccc4ccccc4c3-c3ccc4c(c3)c3ccccc3n4-c3cccc(C4CCCCC4)c3)ccc21]

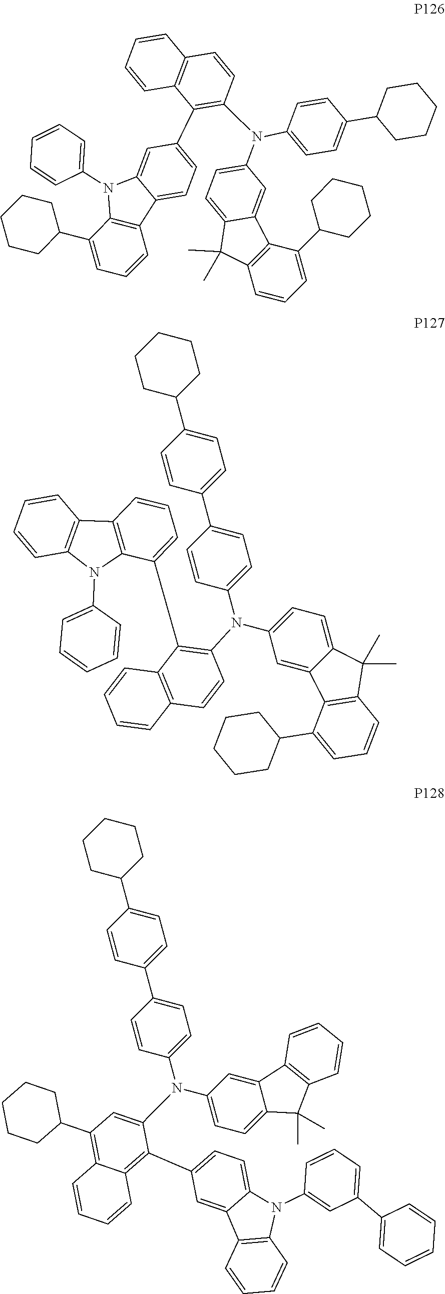 [SMILES: CC1(C)c2ccc(N(c3ccc(C4CCCCC4)cc3)c3ccc4ccccc4c3-c3ccc4c5cccc(C6CCCCC6)c5n(-c5ccccc5)c4c3)cc2-c2c(C3CCCCC3)cccc21 ; CC1(C)c2ccc(N(c3ccc(-c4ccc(C5CCCCC5)cc4)cc3)c3ccc4ccccc4c3-c3cccc4c5ccccc5n(-c5ccccc5)c34)cc2-c2c(C3CCCCC3)cccc21 ; CC1(C)c2ccccc2-c2cc(N(c3ccc(-c4ccc(C5CCCCC5)cc4)cc3)c3cc(C4CCCCC4)c4ccccc4c3-c3ccc4c(c3)c3ccccc3n4-c3cccc(-c4ccccc4)c3)ccc21]

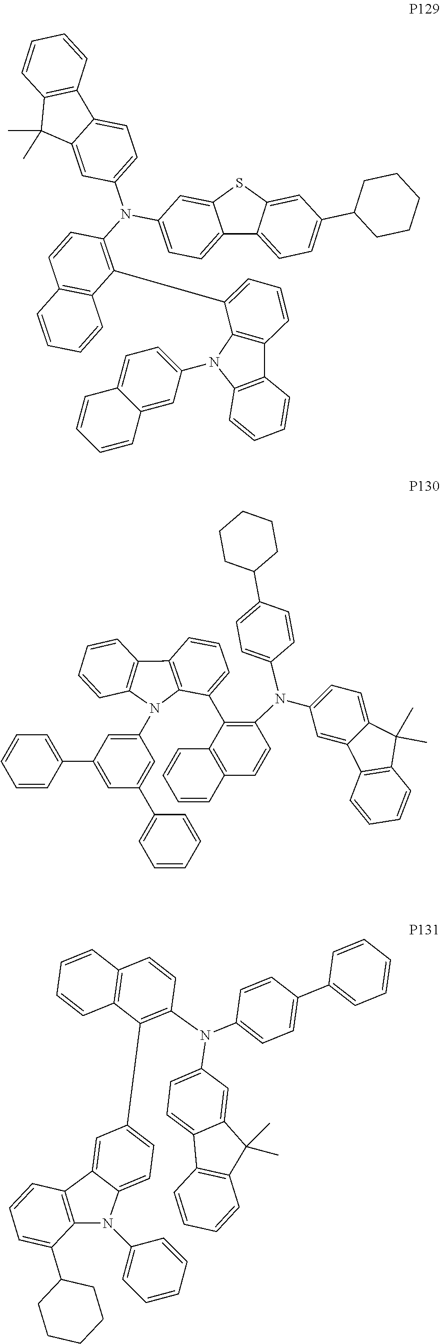 [SMILES: CC1(C)c2ccccc2-c2ccc(N(c3ccc4c(c3)sc3cc(C5CCCCC5)ccc34)c3ccc4ccccc4c3-c3cccc4c5ccccc5n(-c5ccc6ccccc6c5)c34)cc21 ; CC1(C)c2ccccc2-c2cc(N(c3ccc(C4CCCCC4)cc3)c3ccc4ccccc4c3-c3cccc4c5ccccc5n(-c5cc(-c6ccccc6)cc(-c6ccccc6)c5)c34)ccc21 ; CC1(C)c2ccccc2-c2ccc(N(c3ccc(-c4ccccc4)cc3)c3ccc4ccccc4c3-c3ccc4c(c3)c3cccc(C5CCCCC5)c3n4-c3ccccc3)cc21]

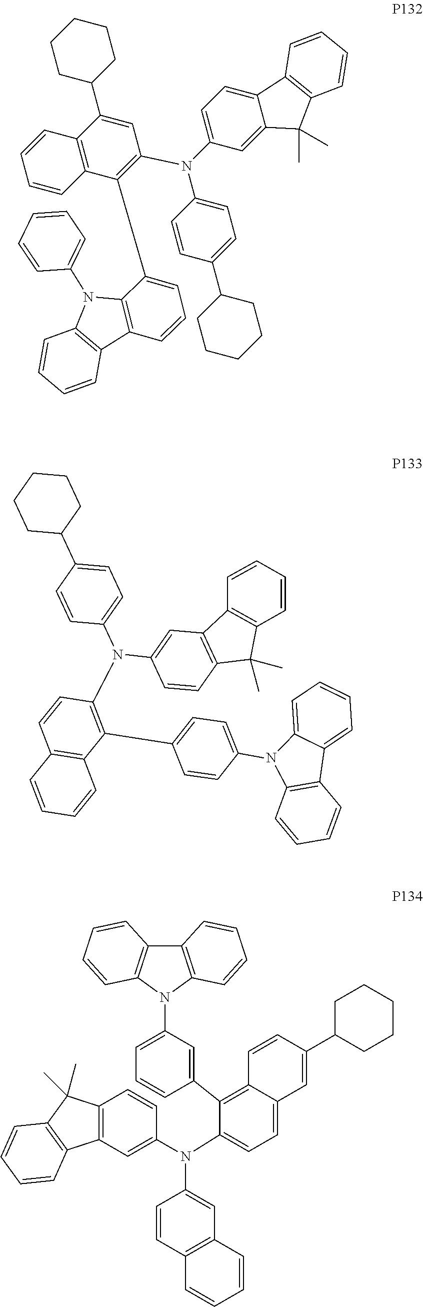 [SMILES: CC1(C)c2ccccc2-c2ccc(N(c3ccc(C4CCCCC4)cc3)c3cc(C4CCCCC4)c4ccccc4c3-c3cccc4c5ccccc5n(-c5ccccc5)c34)cc21 ; CC1(C)c2ccccc2-c2cc(N(c3ccc(C4CCCCC4)cc3)c3ccc4ccccc4c3-c3ccc(-n4c5ccccc5c5ccccc54)cc3)ccc21 ; CC1(C)c2ccccc2-c2cc(N(c3ccc4ccccc4c3)c3ccc4cc(C5CCCCC5)ccc4c3-c3cccc(-n4c5ccccc5c5ccccc54)c3)ccc21]

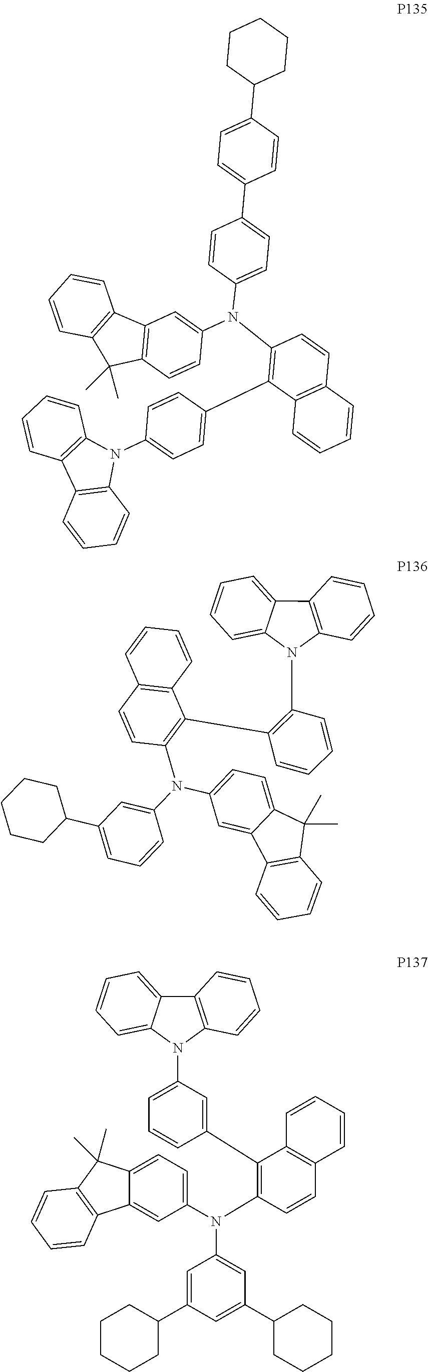 [SMILES: CC1(C)c2ccccc2-c2cc(N(c3ccc(-c4ccc(C5CCCCC5)cc4)cc3)c3ccc4ccccc4c3-c3ccc(-n4c5ccccc5c5ccccc54)cc3)ccc21 ; CC1(C)c2ccccc2-c2cc(N(c3cccc(C4CCCCC4)c3)c3ccc4ccccc4c3-c3ccccc3-n3c4ccccc4c4ccccc43)ccc21 ; CC1(C)c2ccccc2-c2cc(N(c3cc(C4CCCCC4)cc(C4CCCCC4)c3)c3ccc4ccccc4c3-c3cccc(-n4c5ccccc5c5ccccc54)c3)ccc21]

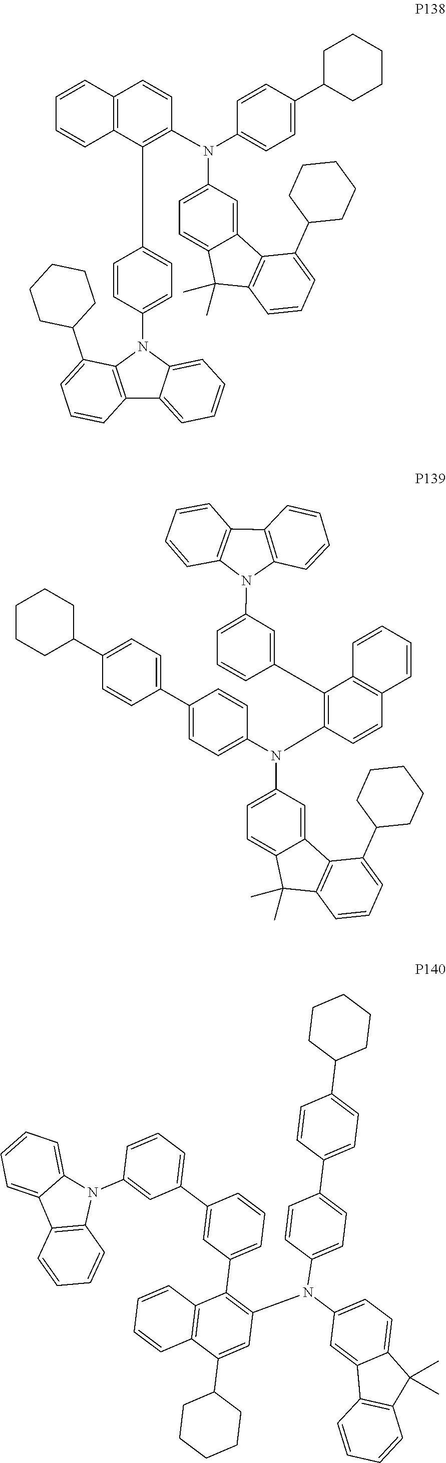 [SMILES: CC1(C)c2ccc(N(c3ccc(C4CCCCC4)cc3)c3ccc4ccccc4c3-c3ccc(-n4c5ccccc5c5cccc(C6CCCCC6)c54)cc3)cc2-c2c(C3CCCCC3)cccc21 ; CC1(C)c2ccc(N(c3ccc(-c4ccc(C5CCCCC5)cc4)cc3)c3ccc4ccccc4c3-c3cccc(-n4c5ccccc5c5ccccc54)c3)cc2-c2c(C3CCCCC3)cccc21 ; CC1(C)c2ccccc2-c2cc(N(c3ccc(-c4ccc(C5CCCCC5)cc4)cc3)c3cc(C4CCCCC4)c4ccccc4c3-c3cccc(-c4cccc(-n5c6ccccc6c6ccccc65)c4)c3)ccc21]

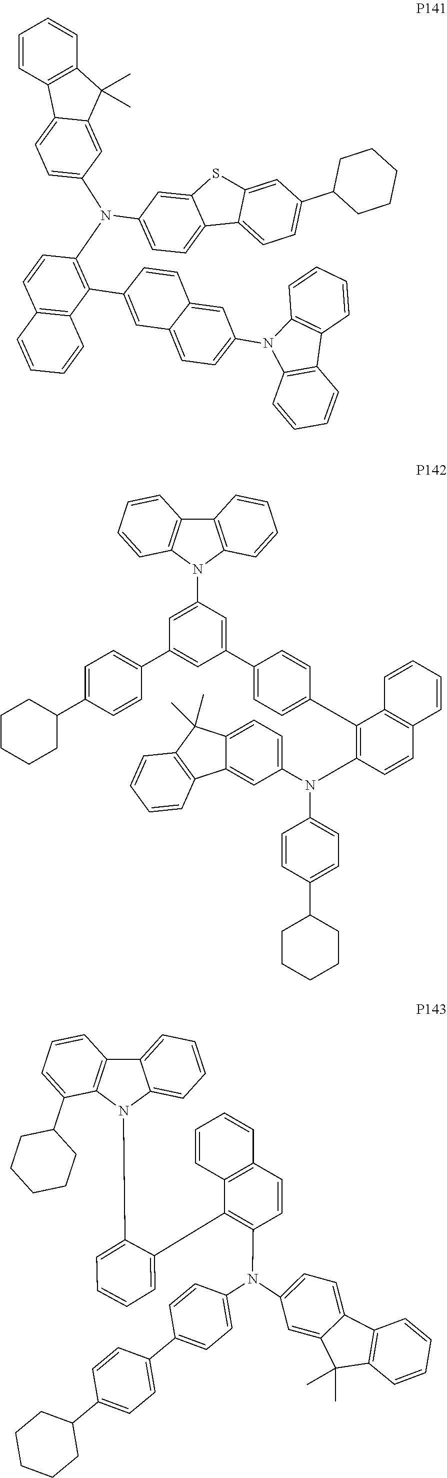 [SMILES: CC1(C)c2ccccc2-c2ccc(N(c3ccc4c(c3)sc3cc(C5CCCCC5)ccc34)c3ccc4ccccc4c3-c3ccc4cc(-n5c6ccccc6c6ccccc65)ccc4c3)cc21 ; CC1(C)c2ccccc2-c2cc(N(c3ccc(C4CCCCC4)cc3)c3ccc4ccccc4c3-c3ccc(-c4cc(-c5ccc(C6CCCCC6)cc5)cc(-n5c6ccccc6c6ccccc65)c4)cc3)ccc21 ; CC1(C)c2ccccc2-c2ccc(N(c3ccc(-c4ccc(C5CCCCC5)cc4)cc3)c3ccc4ccccc4c3-c3ccccc3-n3c4ccccc4c4cccc(C5CCCCC5)c43)cc21]

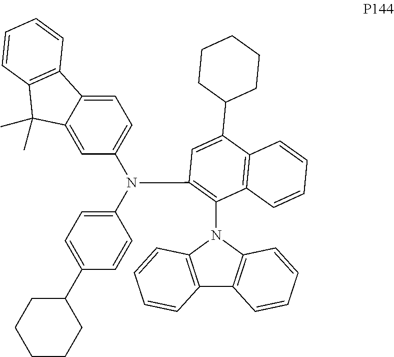 [SMILES: CC1(C)c2ccccc2-c2ccc(N(c3ccc(C4CCCCC4)cc3)c3cc(C4CCCCC4)c4ccccc4c3-n3c4ccccc4c4ccccc43)cc21]

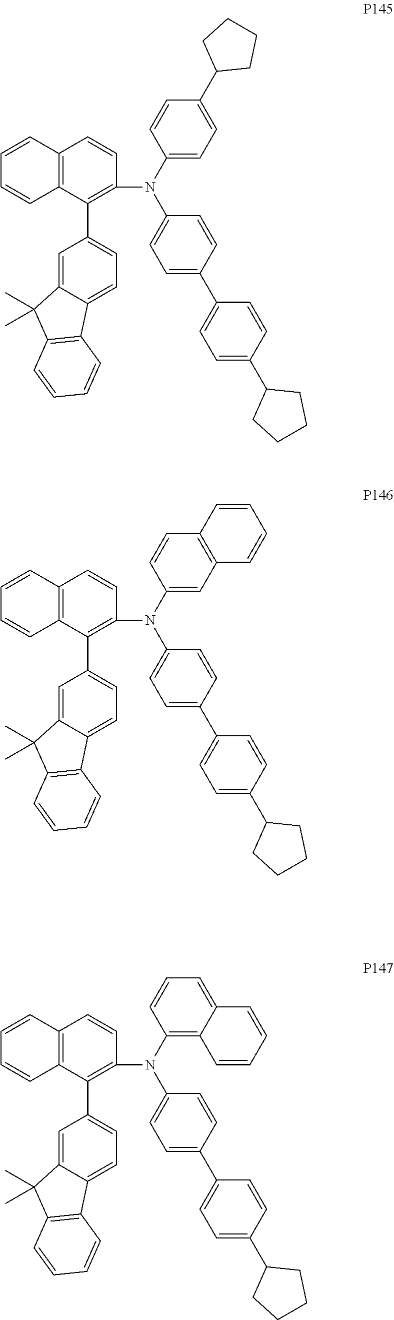 [SMILES: CC1(C)c2ccccc2-c2ccc(-c3c(N(c4ccc(-c5ccc(C6CCCC6)cc5)cc4)c4ccc(C5CCCC5)cc4)ccc4ccccc34)cc21 ; CC1(C)c2ccccc2-c2ccc(-c3c(N(c4ccc(-c5ccc(C6CCCC6)cc5)cc4)c4ccc5ccccc5c4)ccc4ccccc34)cc21 ; CC1(C)c2ccccc2-c2ccc(-c3c(N(c4ccc(-c5ccc(C6CCCC6)cc5)cc4)c4cccc5ccccc45)ccc4ccccc34)cc21]

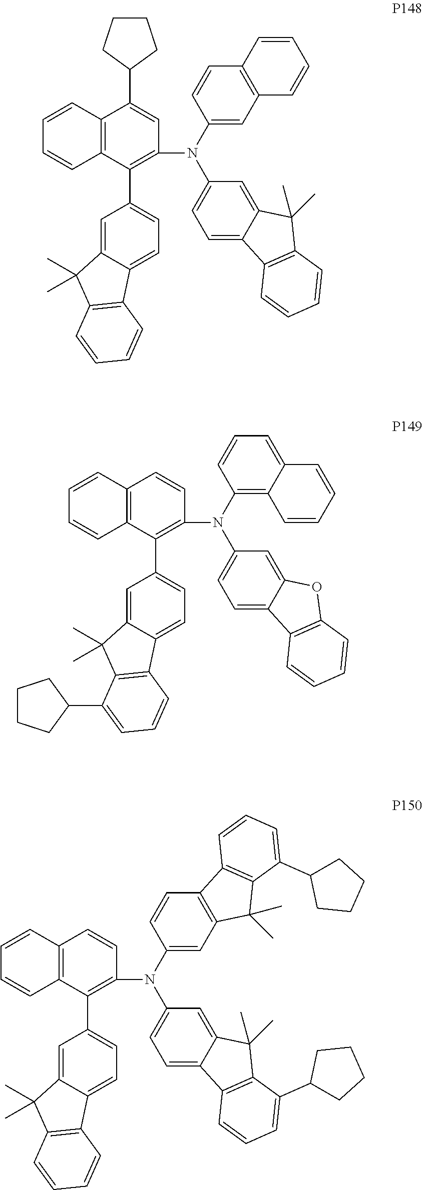 [SMILES: CC1(C)c2ccccc2-c2ccc(-c3c(N(c4ccc5c(c4)C(C)(C)c4ccccc4-5)c4ccc5ccccc5c4)cc(C4CCCC4)c4ccccc34)cc21 ; CC1(C)c2cc(-c3c(N(c4ccc5c(c4)oc4ccccc45)c4cccc5ccccc45)ccc4ccccc34)ccc2-c2cccc(C3CCCC3)c21 ; CC1(C)c2ccccc2-c2ccc(-c3c(N(c4ccc5c(c4)C(C)(C)c4c-5cccc4C4CCCC4)c4ccc5c(c4)C(C)(C)c4c-5cccc4C4CCCC4)ccc4ccccc34)cc21]

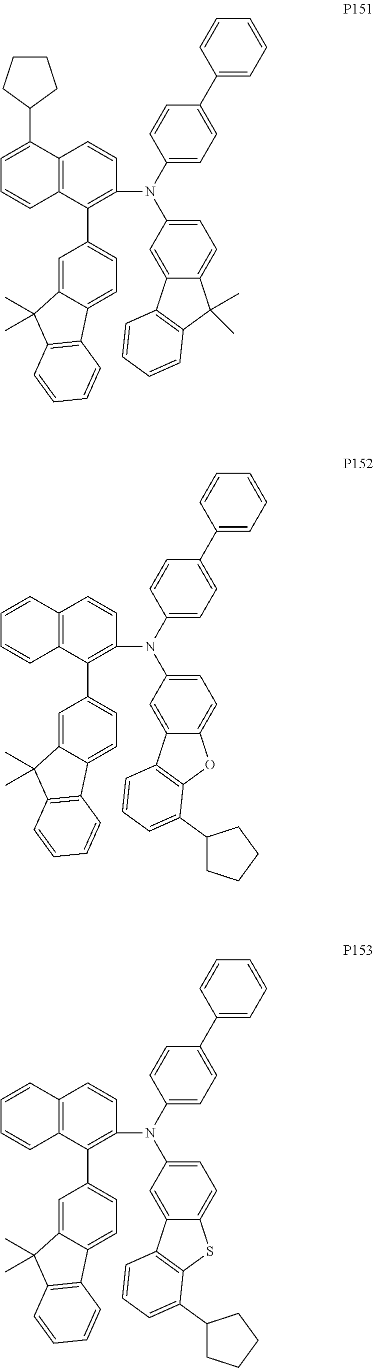 [SMILES: CC1(C)c2ccccc2-c2cc(N(c3ccc(-c4ccccc4)cc3)c3ccc4c(C5CCCC5)cccc4c3-c3ccc4c(c3)C(C)(C)c3ccccc3-4)ccc21 ; CC1(C)c2ccccc2-c2ccc(-c3c(N(c4ccc(-c5ccccc5)cc4)c4ccc5oc6c(C7CCCC7)cccc6c5c4)ccc4ccccc34)cc21 ; CC1(C)c2ccccc2-c2ccc(-c3c(N(c4ccc(-c5ccccc5)cc4)c4ccc5sc6c(C7CCCC7)cccc6c5c4)ccc4ccccc34)cc21]

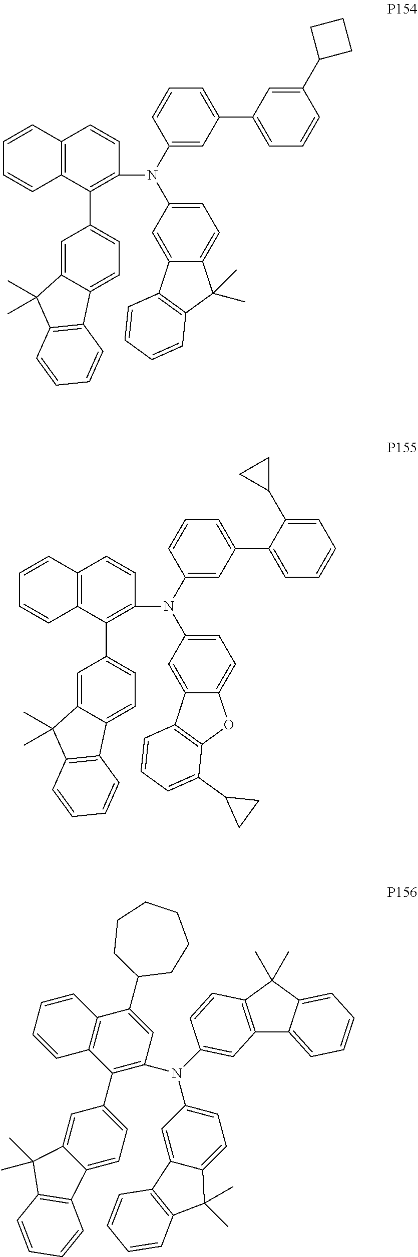 [SMILES: CC1(C)c2ccccc2-c2cc(N(c3cccc(-c4cccc(C5CCC5)c4)c3)c3ccc4ccccc4c3-c3ccc4c(c3)C(C)(C)c3ccccc3-4)ccc21 ; CC1(C)c2ccccc2-c2ccc(-c3c(N(c4cccc(-c5ccccc5C5CC5)c4)c4ccc5oc6c(C7CC7)cccc6c5c4)ccc4ccccc34)cc21 ; CC1(C)c2ccccc2-c2cc(N(c3ccc4c(c3)-c3ccccc3C4(C)C)c3cc(C4CCCCCC4)c4ccccc4c3-c3ccc4c(c3)C(C)(C)c3ccccc3-4)ccc21]

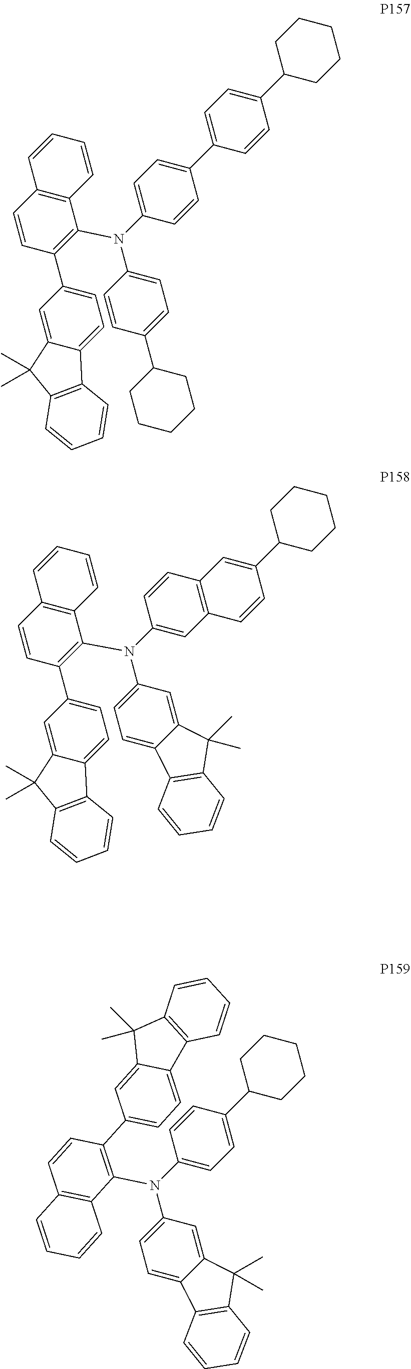 [SMILES: CC1(C)c2ccccc2-c2ccc(-c3ccc4ccccc4c3N(c3ccc(-c4ccc(C5CCCCC5)cc4)cc3)c3ccc(C4CCCCC4)cc3)cc21 ; CC1(C)c2ccccc2-c2ccc(-c3ccc4ccccc4c3N(c3ccc4c(c3)C(C)(C)c3ccccc3-4)c3ccc4cc(C5CCCCC5)ccc4c3)cc21 ; CC1(C)c2ccccc2-c2ccc(-c3ccc4ccccc4c3N(c3ccc(C4CCCCC4)cc3)c3ccc4c(c3)C(C)(C)c3ccccc3-4)cc21]

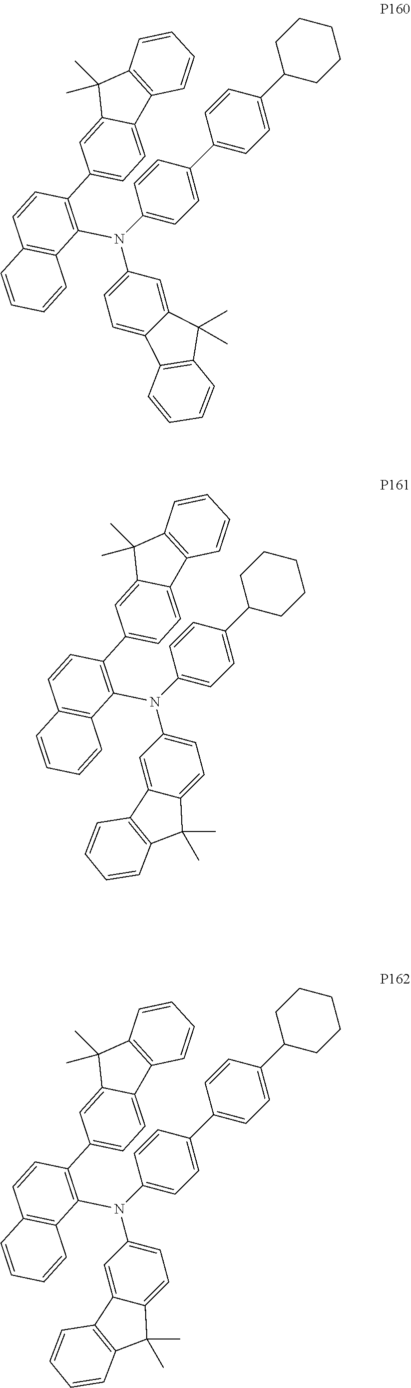 [SMILES: CC1(C)c2ccccc2-c2ccc(-c3ccc4ccccc4c3N(c3ccc(-c4ccc(C5CCCCC5)cc4)cc3)c3ccc4c(c3)C(C)(C)c3ccccc3-4)cc21 ; CC1(C)c2ccccc2-c2cc(N(c3ccc(C4CCCCC4)cc3)c3c(-c4ccc5c(c4)C(C)(C)c4ccccc4-5)ccc4ccccc34)ccc21 ; CC1(C)c2ccccc2-c2cc(N(c3ccc(-c4ccc(C5CCCCC5)cc4)cc3)c3c(-c4ccc5c(c4)C(C)(C)c4ccccc4-5)ccc4ccccc34)ccc21]

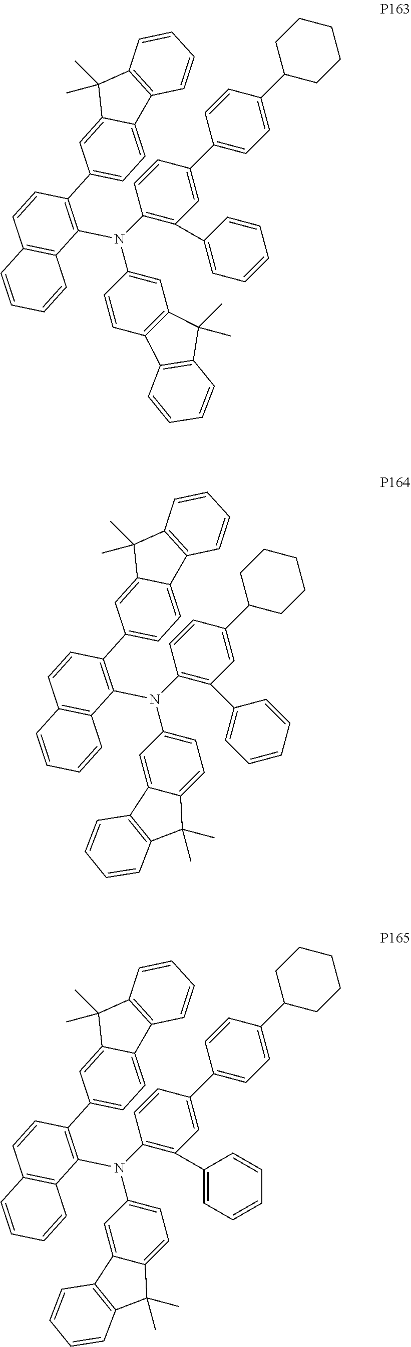 [SMILES: CC1(C)c2ccccc2-c2ccc(-c3ccc4ccccc4c3N(c3ccc4c(c3)C(C)(C)c3ccccc3-4)c3ccc(-c4ccc(C5CCCCC5)cc4)cc3-c3ccccc3)cc21 ; CC1(C)c2ccccc2-c2cc(N(c3ccc(C4CCCCC4)cc3-c3ccccc3)c3c(-c4ccc5c(c4)C(C)(C)c4ccccc4-5)ccc4ccccc34)ccc21 ; CC1(C)c2ccccc2-c2cc(N(c3ccc(-c4ccc(C5CCCCC5)cc4)cc3-c3ccccc3)c3c(-c4ccc5c(c4)C(C)(C)c4ccccc4-5)ccc4ccccc34)ccc21]

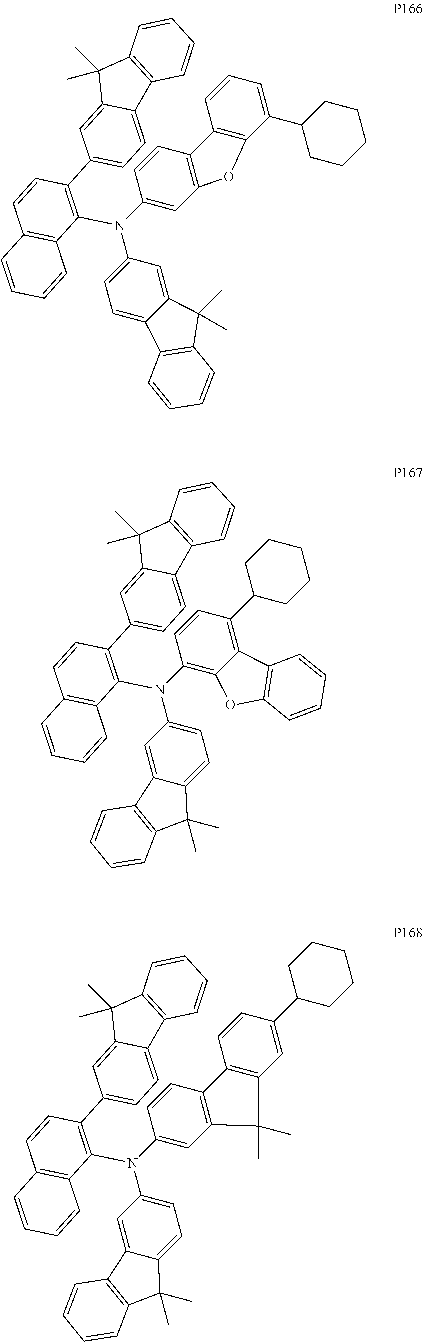 [SMILES: CC1(C)c2ccccc2-c2ccc(-c3ccc4ccccc4c3N(c3ccc4c(c3)C(C)(C)c3ccccc3-4)c3ccc4c(c3)oc3c(C5CCCCC5)cccc34)cc21 ; CC1(C)c2ccccc2-c2cc(N(c3c(-c4ccc5c(c4)C(C)(C)c4ccccc4-5)ccc4ccccc34)c3ccc(C4CCCCC4)c4c3oc3ccccc34)ccc21 ; CC1(C)c2ccccc2-c2cc(N(c3ccc4c(c3)C(C)(C)c3cc(C5CCCCC5)ccc3-4)c3c(-c4ccc5c(c4)C(C)(C)c4ccccc4-5)ccc4ccccc34)ccc21]

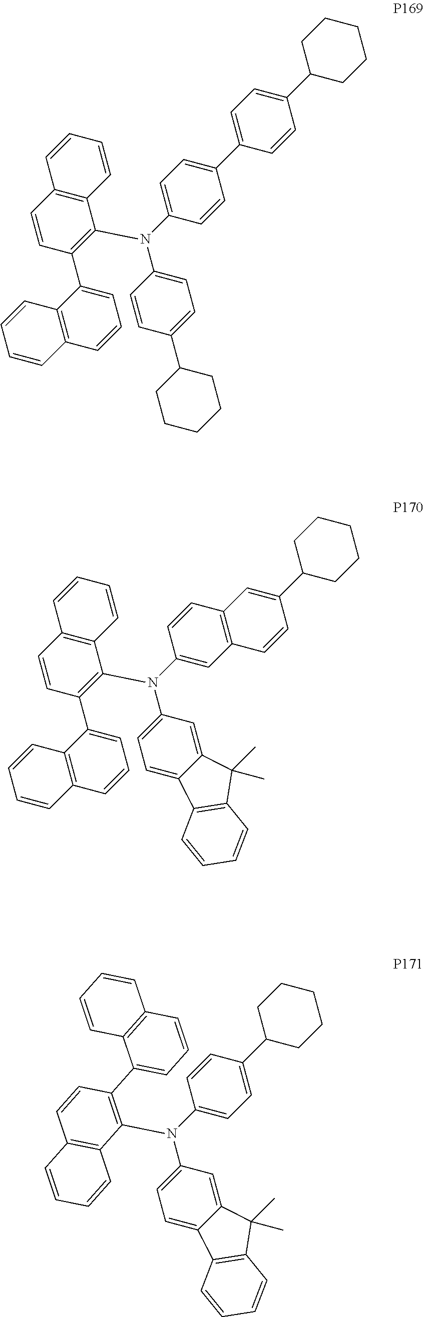 [SMILES: c1ccc2c(-c3ccc4ccccc4c3N(c3ccc(-c4ccc(C5CCCCC5)cc4)cc3)c3ccc(C4CCCCC4)cc3)cccc2c1 ; CC1(C)c2ccccc2-c2ccc(N(c3ccc4cc(C5CCCCC5)ccc4c3)c3c(-c4cccc5ccccc45)ccc4ccccc34)cc21 ; CC1(C)c2ccccc2-c2ccc(N(c3ccc(C4CCCCC4)cc3)c3c(-c4cccc5ccccc45)ccc4ccccc34)cc21]

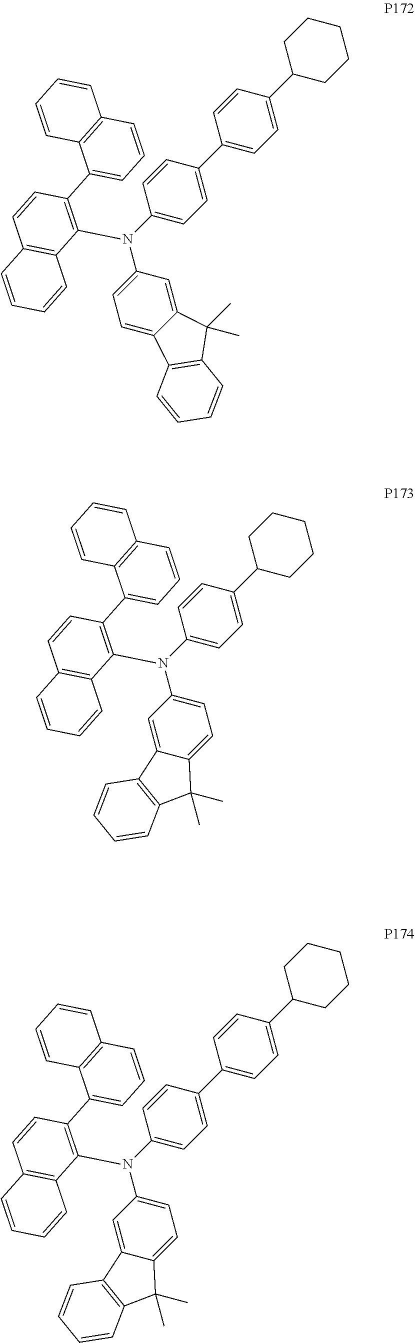 [SMILES: CC1(C)c2ccccc2-c2ccc(N(c3ccc(-c4ccc(C5CCCCC5)cc4)cc3)c3c(-c4cccc5ccccc45)ccc4ccccc34)cc21 ; CC1(C)c2ccccc2-c2cc(N(c3ccc(C4CCCCC4)cc3)c3c(-c4cccc5ccccc45)ccc4ccccc34)ccc21 ; CC1(C)c2ccccc2-c2cc(N(c3ccc(-c4ccc(C5CCCCC5)cc4)cc3)c3c(-c4cccc5ccccc45)ccc4ccccc34)ccc21]

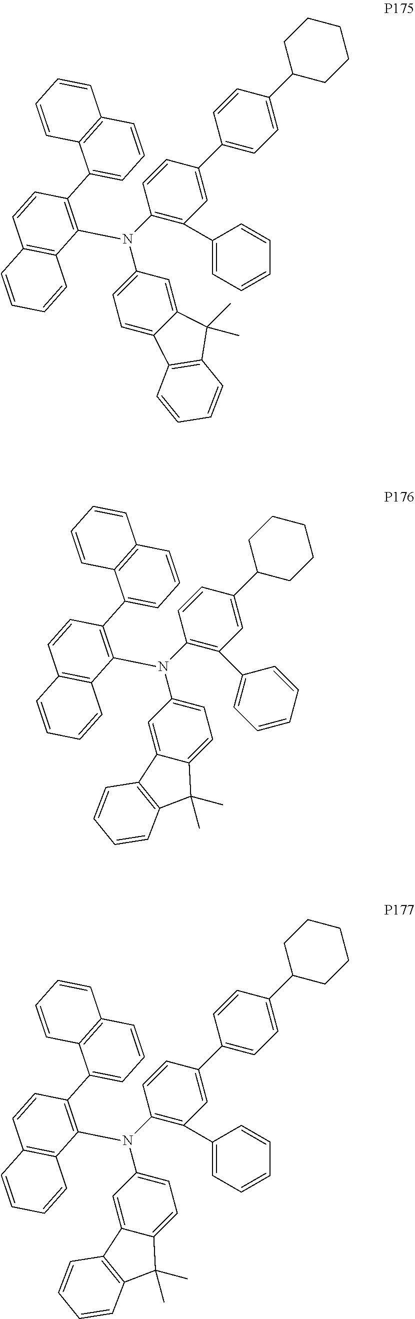 [SMILES: CC1(C)c2ccccc2-c2ccc(N(c3ccc(-c4ccc(C5CCCCC5)cc4)cc3-c3ccccc3)c3c(-c4cccc5ccccc45)ccc4ccccc34)cc21 ; CC1(C)c2ccccc2-c2cc(N(c3ccc(C4CCCCC4)cc3-c3ccccc3)c3c(-c4cccc5ccccc45)ccc4ccccc34)ccc21 ; CC1(C)c2ccccc2-c2cc(N(c3ccc(-c4ccc(C5CCCCC5)cc4)cc3-c3ccccc3)c3c(-c4cccc5ccccc45)ccc4ccccc34)ccc21]

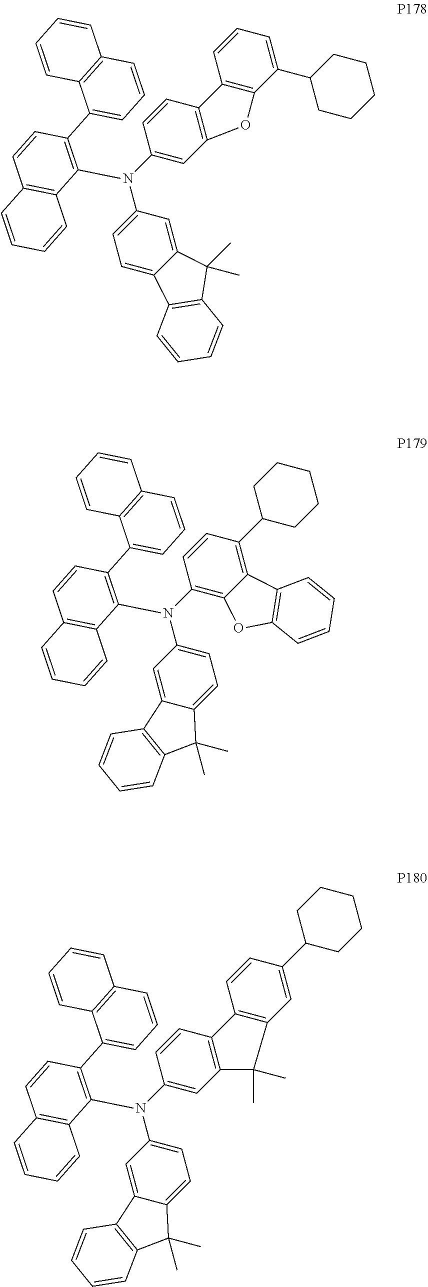 [SMILES: CC1(C)c2ccccc2-c2ccc(N(c3ccc4c(c3)oc3c(C5CCCCC5)cccc34)c3c(-c4cccc5ccccc45)ccc4ccccc34)cc21 ; CC1(C)c2ccccc2-c2cc(N(c3c(-c4cccc5ccccc45)ccc4ccccc34)c3ccc(C4CCCCC4)c4c3oc3ccccc34)ccc21 ; CC1(C)c2ccccc2-c2cc(N(c3ccc4c(c3)C(C)(C)c3cc(C5CCCCC5)ccc3-4)c3c(-c4cccc5ccccc45)ccc4ccccc34)ccc21]

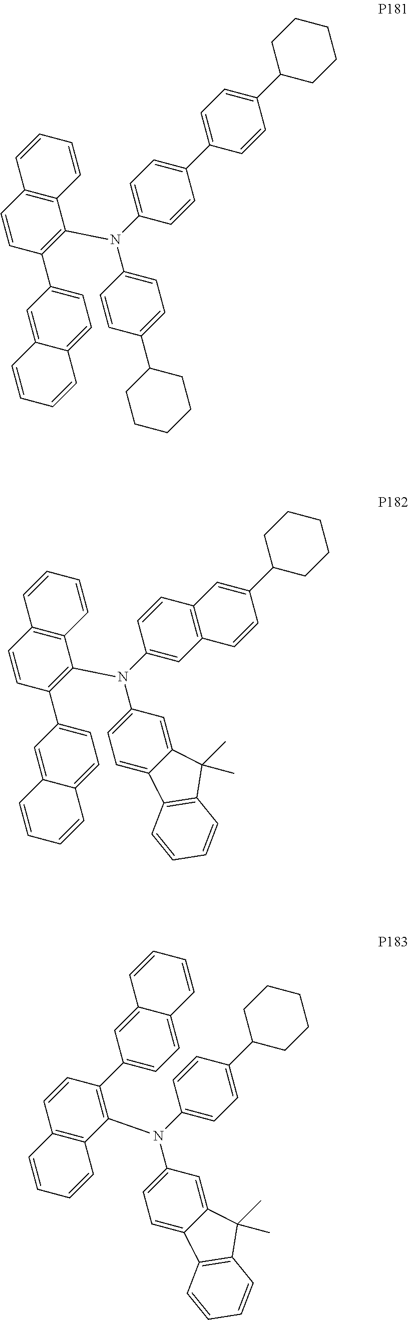 [SMILES: c1ccc2cc(-c3ccc4ccccc4c3N(c3ccc(-c4ccc(C5CCCCC5)cc4)cc3)c3ccc(C4CCCCC4)cc3)ccc2c1 ; CC1(C)c2ccccc2-c2ccc(N(c3ccc4cc(C5CCCCC5)ccc4c3)c3c(-c4ccc5ccccc5c4)ccc4ccccc34)cc21 ; CC1(C)c2ccccc2-c2ccc(N(c3ccc(C4CCCCC4)cc3)c3c(-c4ccc5ccccc5c4)ccc4ccccc34)cc21]

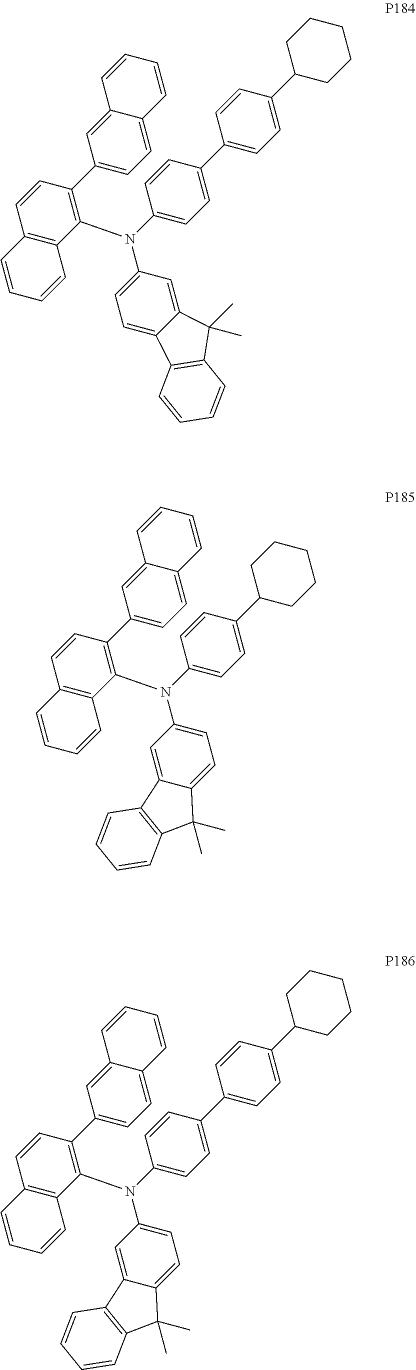 [SMILES: CC1(C)c2ccccc2-c2ccc(N(c3ccc(-c4ccc(C5CCCCC5)cc4)cc3)c3c(-c4ccc5ccccc5c4)ccc4ccccc34)cc21 ; CC1(C)c2ccccc2-c2cc(N(c3ccc(C4CCCCC4)cc3)c3c(-c4ccc5ccccc5c4)ccc4ccccc34)ccc21 ; CC1(C)c2ccccc2-c2cc(N(c3ccc(-c4ccc(C5CCCCC5)cc4)cc3)c3c(-c4ccc5ccccc5c4)ccc4ccccc34)ccc21]

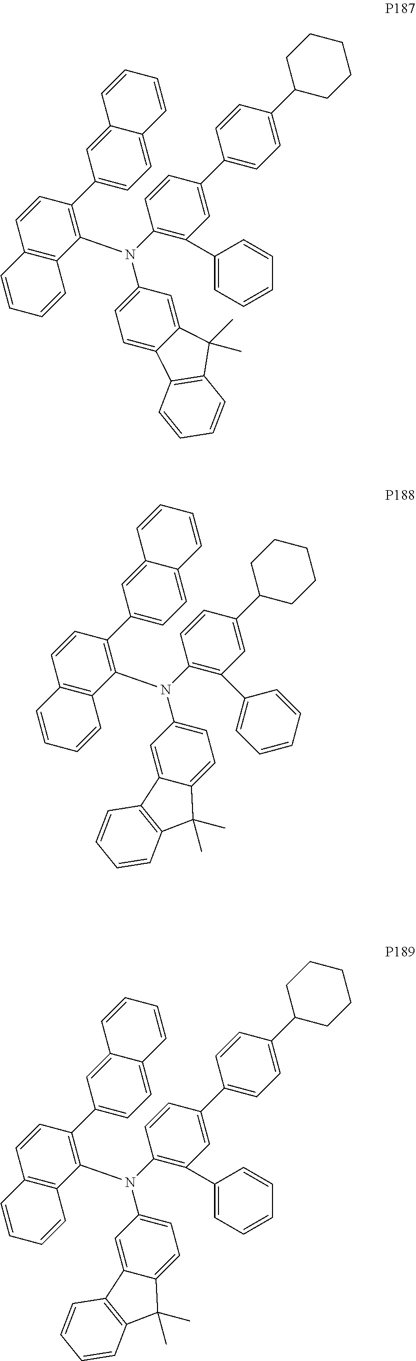 [SMILES: CC1(C)c2ccccc2-c2ccc(N(c3ccc(-c4ccc(C5CCCCC5)cc4)cc3-c3ccccc3)c3c(-c4ccc5ccccc5c4)ccc4ccccc34)cc21 ; CC1(C)c2ccccc2-c2cc(N(c3ccc(C4CCCCC4)cc3-c3ccccc3)c3c(-c4ccc5ccccc5c4)ccc4ccccc34)ccc21 ; CC1(C)c2ccccc2-c2cc(N(c3ccc(-c4ccc(C5CCCCC5)cc4)cc3-c3ccccc3)c3c(-c4ccc5ccccc5c4)ccc4ccccc34)ccc21]

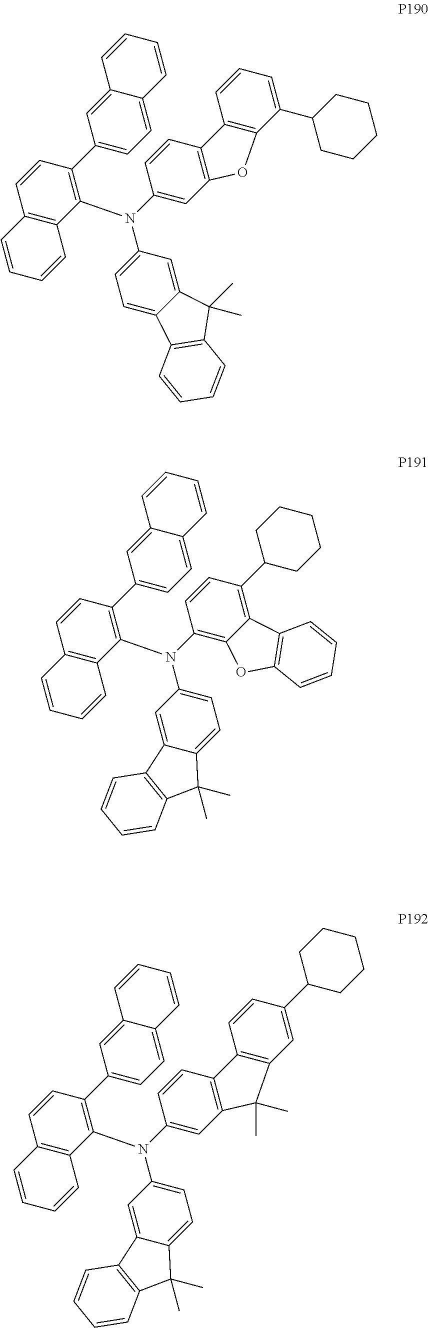 [SMILES: CC1(C)c2ccccc2-c2ccc(N(c3ccc4c(c3)oc3c(C5CCCCC5)cccc34)c3c(-c4ccc5ccccc5c4)ccc4ccccc34)cc21 ; CC1(C)c2ccccc2-c2cc(N(c3c(-c4ccc5ccccc5c4)ccc4ccccc34)c3ccc(C4CCCCC4)c4c3oc3ccccc34)ccc21 ; CC1(C)c2ccccc2-c2cc(N(c3ccc4c(c3)C(C)(C)c3cc(C5CCCCC5)ccc3-4)c3c(-c4ccc5ccccc5c4)ccc4ccccc34)ccc21]

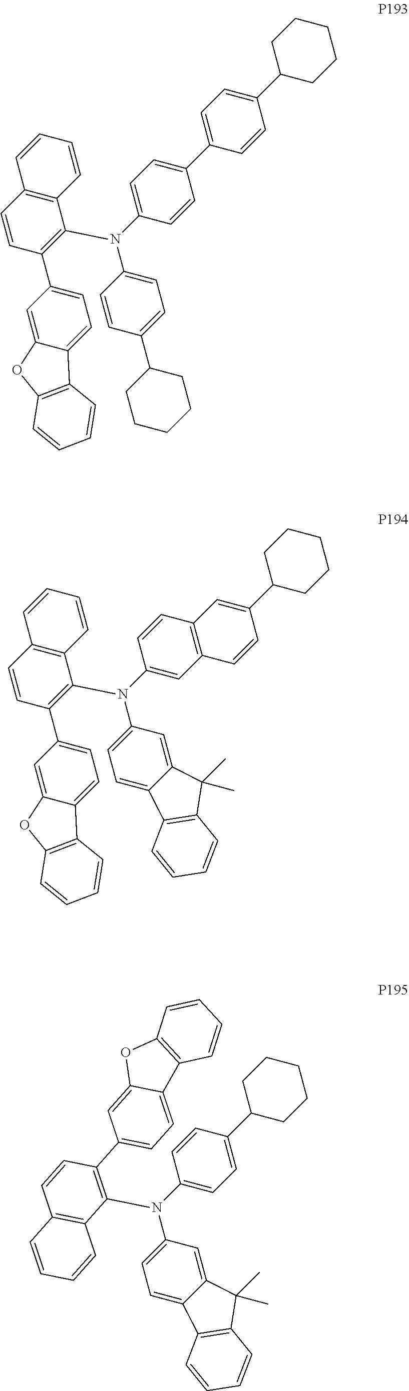 [SMILES: c1ccc2c(N(c3ccc(-c4ccc(C5CCCCC5)cc4)cc3)c3ccc(C4CCCCC4)cc3)c(-c3ccc4c(c3)oc3ccccc34)ccc2c1 ; CC1(C)c2ccccc2-c2ccc(N(c3ccc4cc(C5CCCCC5)ccc4c3)c3c(-c4ccc5c(c4)oc4ccccc45)ccc4ccccc34)cc21 ; CC1(C)c2ccccc2-c2ccc(N(c3ccc(C4CCCCC4)cc3)c3c(-c4ccc5c(c4)oc4ccccc45)ccc4ccccc34)cc21]

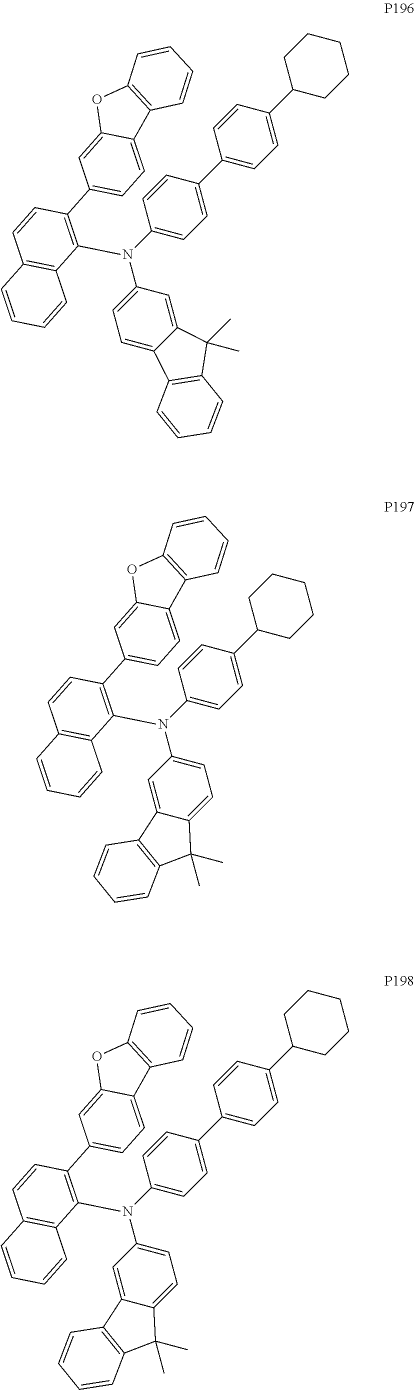 [SMILES: CC1(C)c2ccccc2-c2ccc(N(c3ccc(-c4ccc(C5CCCCC5)cc4)cc3)c3c(-c4ccc5c(c4)oc4ccccc45)ccc4ccccc34)cc21 ; CC1(C)c2ccccc2-c2cc(N(c3ccc(C4CCCCC4)cc3)c3c(-c4ccc5c(c4)oc4ccccc45)ccc4ccccc34)ccc21 ; CC1(C)c2ccccc2-c2cc(N(c3ccc(-c4ccc(C5CCCCC5)cc4)cc3)c3c(-c4ccc5c(c4)oc4ccccc45)ccc4ccccc34)ccc21]

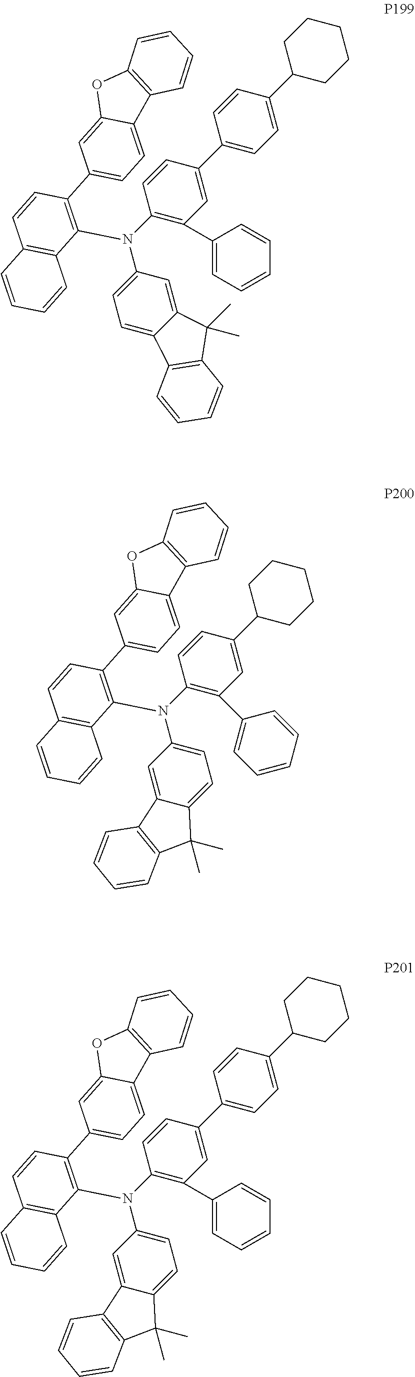 [SMILES: CC1(C)c2ccccc2-c2ccc(N(c3ccc(-c4ccc(C5CCCCC5)cc4)cc3-c3ccccc3)c3c(-c4ccc5c(c4)oc4ccccc45)ccc4ccccc34)cc21 ; CC1(C)c2ccccc2-c2cc(N(c3ccc(C4CCCCC4)cc3-c3ccccc3)c3c(-c4ccc5c(c4)oc4ccccc45)ccc4ccccc34)ccc21 ; CC1(C)c2ccccc2-c2cc(N(c3ccc(-c4ccc(C5CCCCC5)cc4)cc3-c3ccccc3)c3c(-c4ccc5c(c4)oc4ccccc45)ccc4ccccc34)ccc21]

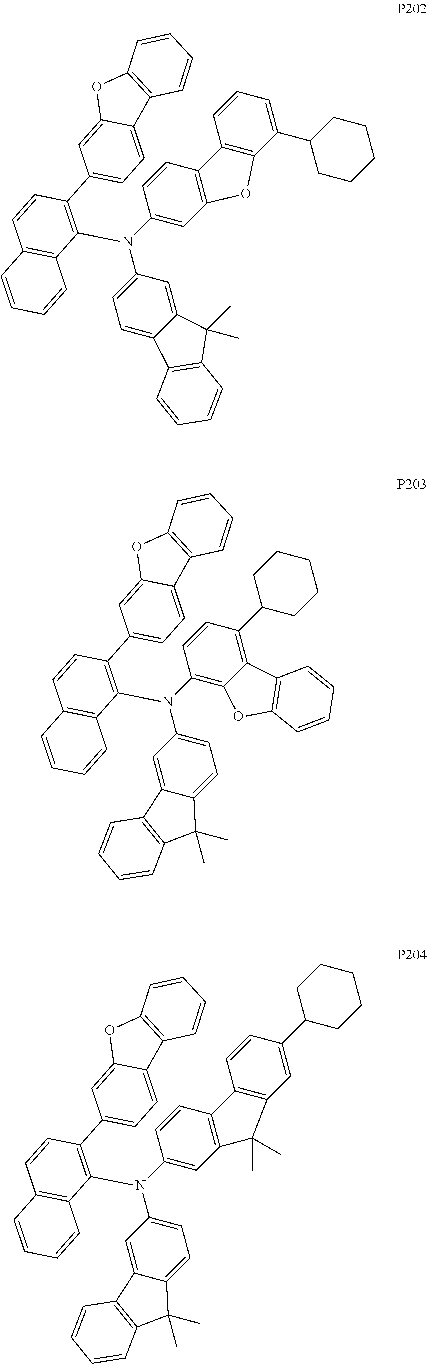 [SMILES: CC1(C)c2ccccc2-c2ccc(N(c3ccc4c(c3)oc3c(C5CCCCC5)cccc34)c3c(-c4ccc5c(c4)oc4ccccc45)ccc4ccccc34)cc21 ; CC1(C)c2ccccc2-c2cc(N(c3c(-c4ccc5c(c4)oc4ccccc45)ccc4ccccc34)c3ccc(C4CCCCC4)c4c3oc3ccccc34)ccc21 ; CC1(C)c2ccccc2-c2cc(N(c3ccc4c(c3)C(C)(C)c3cc(C5CCCCC5)ccc3-4)c3c(-c4ccc5c(c4)oc4ccccc45)ccc4ccccc34)ccc21]

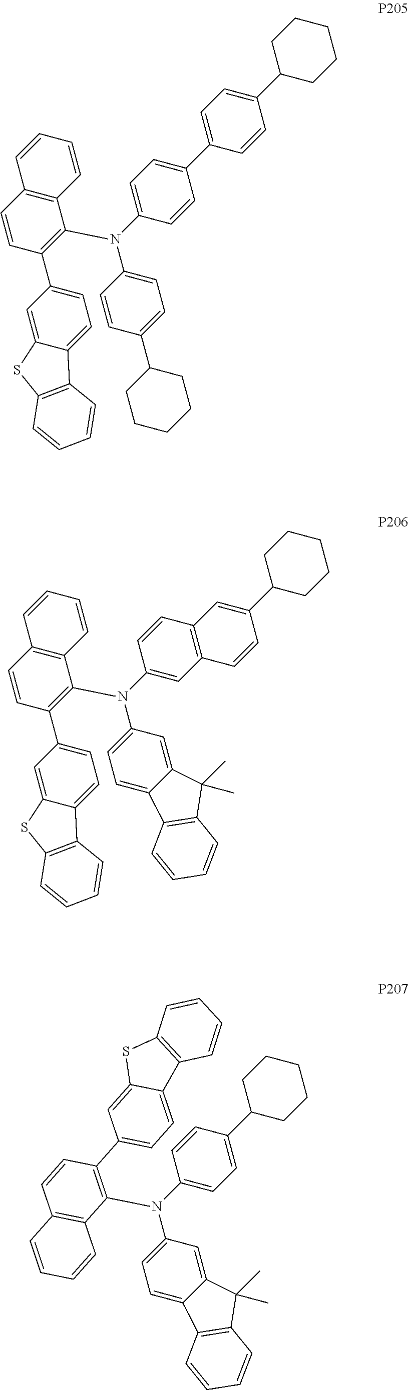 [SMILES: c1ccc2c(N(c3ccc(-c4ccc(C5CCCCC5)cc4)cc3)c3ccc(C4CCCCC4)cc3)c(-c3ccc4c(c3)sc3ccccc34)ccc2c1 ; CC1(C)c2ccccc2-c2ccc(N(c3ccc4cc(C5CCCCC5)ccc4c3)c3c(-c4ccc5c(c4)sc4ccccc45)ccc4ccccc34)cc21 ; CC1(C)c2ccccc2-c2ccc(N(c3ccc(C4CCCCC4)cc3)c3c(-c4ccc5c(c4)sc4ccccc45)ccc4ccccc34)cc21]

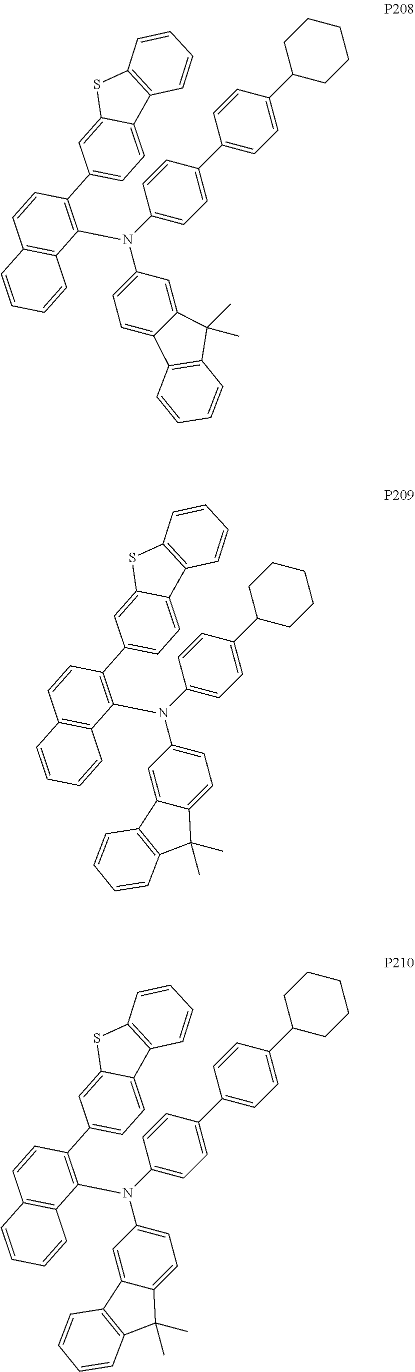 [SMILES: CC1(C)c2ccccc2-c2ccc(N(c3ccc(-c4ccc(C5CCCCC5)cc4)cc3)c3c(-c4ccc5c(c4)sc4ccccc45)ccc4ccccc34)cc21 ; CC1(C)c2ccccc2-c2cc(N(c3ccc(C4CCCCC4)cc3)c3c(-c4ccc5c(c4)sc4ccccc45)ccc4ccccc34)ccc21 ; CC1(C)c2ccccc2-c2cc(N(c3ccc(-c4ccc(C5CCCCC5)cc4)cc3)c3c(-c4ccc5c(c4)sc4ccccc45)ccc4ccccc34)ccc21]

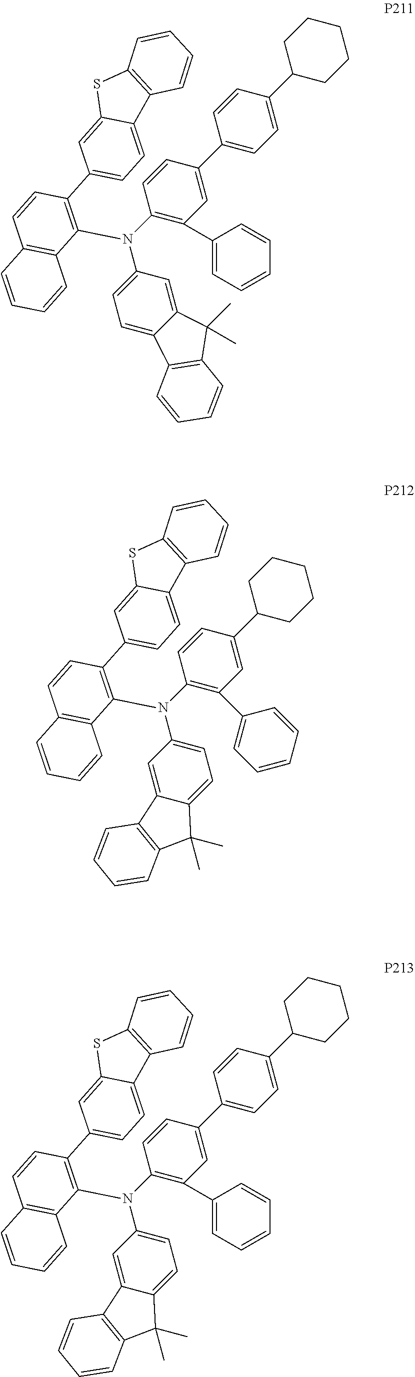 [SMILES: CC1(C)c2ccccc2-c2ccc(N(c3ccc(-c4ccc(C5CCCCC5)cc4)cc3-c3ccccc3)c3c(-c4ccc5c(c4)sc4ccccc45)ccc4ccccc34)cc21 ; CC1(C)c2ccccc2-c2cc(N(c3ccc(C4CCCCC4)cc3-c3ccccc3)c3c(-c4ccc5c(c4)sc4ccccc45)ccc4ccccc34)ccc21 ; CC1(C)c2ccccc2-c2cc(N(c3ccc(-c4ccc(C5CCCCC5)cc4)cc3-c3ccccc3)c3c(-c4ccc5c(c4)sc4ccccc45)ccc4ccccc34)ccc21]

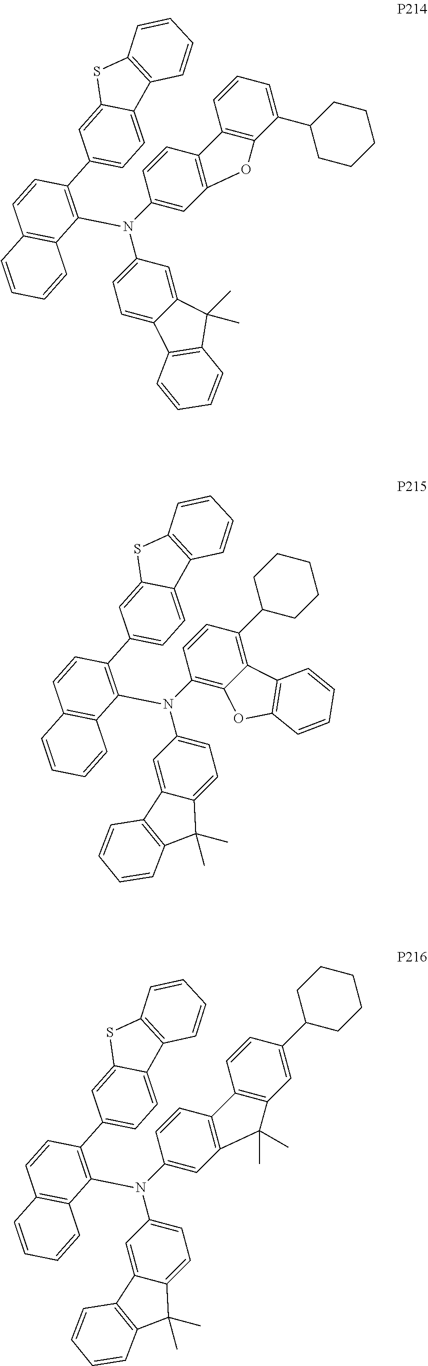 [SMILES: CC1(C)c2ccccc2-c2ccc(N(c3ccc4c(c3)oc3c(C5CCCCC5)cccc34)c3c(-c4ccc5c(c4)sc4ccccc45)ccc4ccccc34)cc21 ; CC1(C)c2ccccc2-c2cc(N(c3c(-c4ccc5c(c4)sc4ccccc45)ccc4ccccc34)c3ccc(C4CCCCC4)c4c3oc3ccccc34)ccc21 ; CC1(C)c2ccccc2-c2cc(N(c3ccc4c(c3)C(C)(C)c3cc(C5CCCCC5)ccc3-4)c3c(-c4ccc5c(c4)sc4ccccc45)ccc4ccccc34)ccc21]

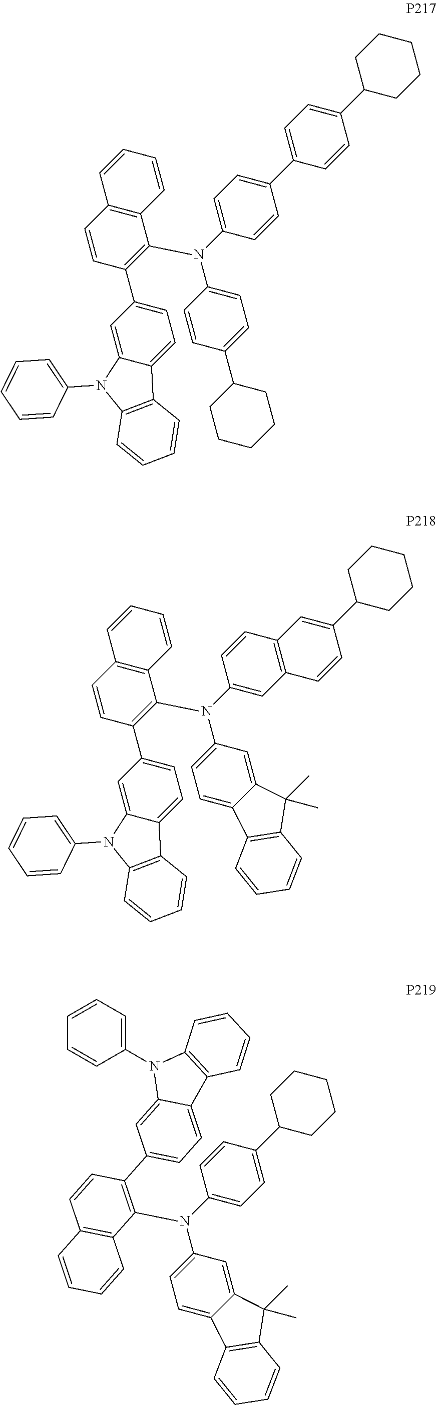 [SMILES: c1ccc(-n2c3ccccc3c3ccc(-c4ccc5ccccc5c4N(c4ccc(-c5ccc(C6CCCCC6)cc5)cc4)c4ccc(C5CCCCC5)cc4)cc32)cc1 ; CC1(C)c2ccccc2-c2ccc(N(c3ccc4cc(C5CCCCC5)ccc4c3)c3c(-c4ccc5c6ccccc6n(-c6ccccc6)c5c4)ccc4ccccc34)cc21 ; CC1(C)c2ccccc2-c2ccc(N(c3ccc(C4CCCCC4)cc3)c3c(-c4ccc5c6ccccc6n(-c6ccccc6)c5c4)ccc4ccccc34)cc21]

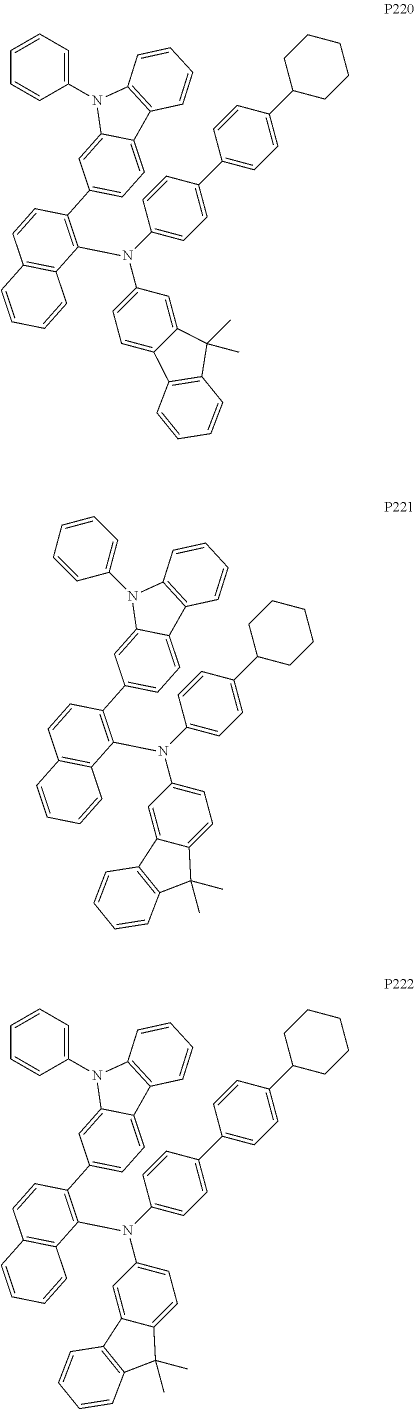 [SMILES: CC1(C)c2ccccc2-c2ccc(N(c3ccc(-c4ccc(C5CCCCC5)cc4)cc3)c3c(-c4ccc5c6ccccc6n(-c6ccccc6)c5c4)ccc4ccccc34)cc21 ; CC1(C)c2ccccc2-c2cc(N(c3ccc(C4CCCCC4)cc3)c3c(-c4ccc5c6ccccc6n(-c6ccccc6)c5c4)ccc4ccccc34)ccc21 ; CC1(C)c2ccccc2-c2cc(N(c3ccc(-c4ccc(C5CCCCC5)cc4)cc3)c3c(-c4ccc5c6ccccc6n(-c6ccccc6)c5c4)ccc4ccccc34)ccc21]

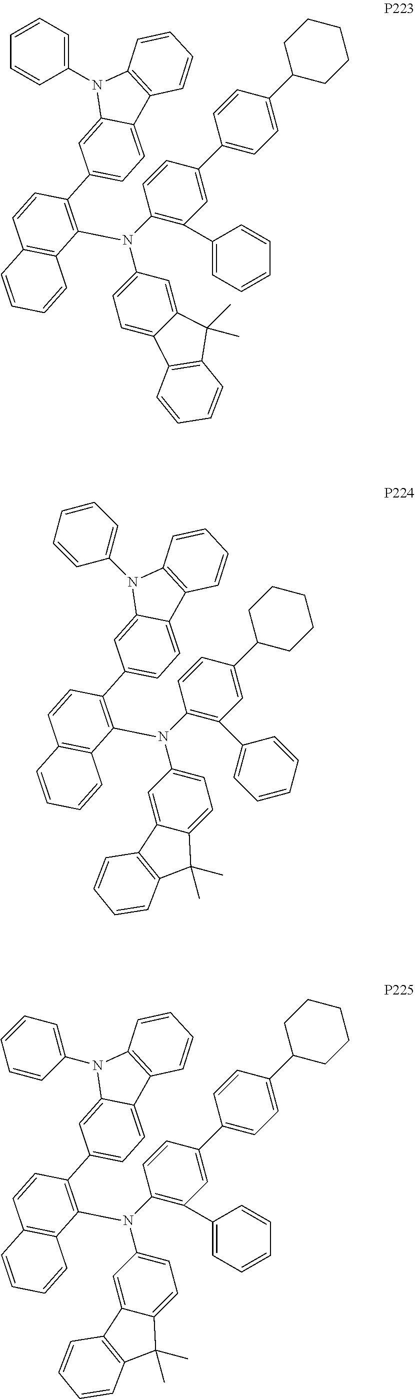 [SMILES: CC1(C)c2ccccc2-c2ccc(N(c3ccc(-c4ccc(C5CCCCC5)cc4)cc3-c3ccccc3)c3c(-c4ccc5c6ccccc6n(-c6ccccc6)c5c4)ccc4ccccc34)cc21 ; CC1(C)c2ccccc2-c2cc(N(c3ccc(C4CCCCC4)cc3-c3ccccc3)c3c(-c4ccc5c6ccccc6n(-c6ccccc6)c5c4)ccc4ccccc34)ccc21 ; CC1(C)c2ccccc2-c2cc(N(c3ccc(-c4ccc(C5CCCCC5)cc4)cc3-c3ccccc3)c3c(-c4ccc5c6ccccc6n(-c6ccccc6)c5c4)ccc4ccccc34)ccc21]

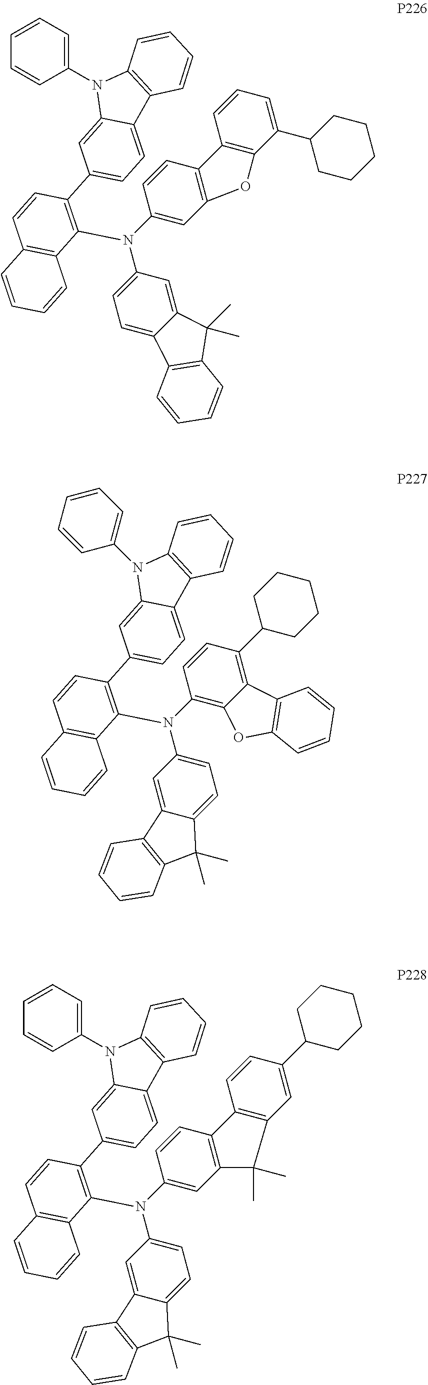 [SMILES: CC1(C)c2ccccc2-c2ccc(N(c3ccc4c(c3)oc3c(C5CCCCC5)cccc34)c3c(-c4ccc5c6ccccc6n(-c6ccccc6)c5c4)ccc4ccccc34)cc21 ; CC1(C)c2ccccc2-c2cc(N(c3c(-c4ccc5c6ccccc6n(-c6ccccc6)c5c4)ccc4ccccc34)c3ccc(C4CCCCC4)c4c3oc3ccccc34)ccc21 ; CC1(C)c2ccccc2-c2cc(N(c3ccc4c(c3)C(C)(C)c3cc(C5CCCCC5)ccc3-4)c3c(-c4ccc5c6ccccc6n(-c6ccccc6)c5c4)ccc4ccccc34)ccc21]

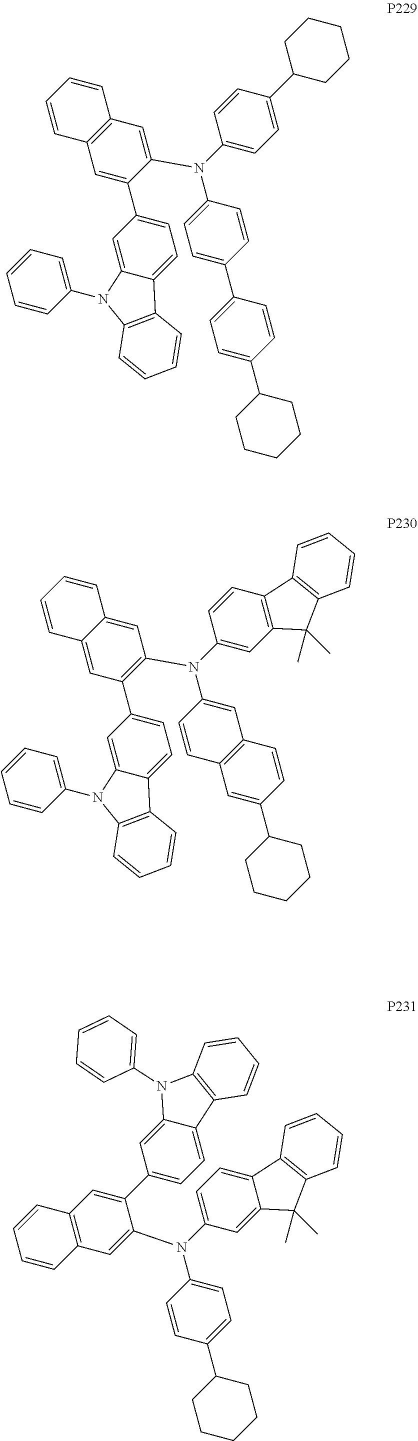 [SMILES: c1ccc(-n2c3ccccc3c3ccc(-c4cc5ccccc5cc4N(c4ccc(-c5ccc(C6CCCCC6)cc5)cc4)c4ccc(C5CCCCC5)cc4)cc32)cc1 ; CC1(C)c2ccccc2-c2ccc(N(c3ccc4cc(C5CCCCC5)ccc4c3)c3cc4ccccc4cc3-c3ccc4c5ccccc5n(-c5ccccc5)c4c3)cc21 ; CC1(C)c2ccccc2-c2ccc(N(c3ccc(C4CCCCC4)cc3)c3cc4ccccc4cc3-c3ccc4c5ccccc5n(-c5ccccc5)c4c3)cc21]

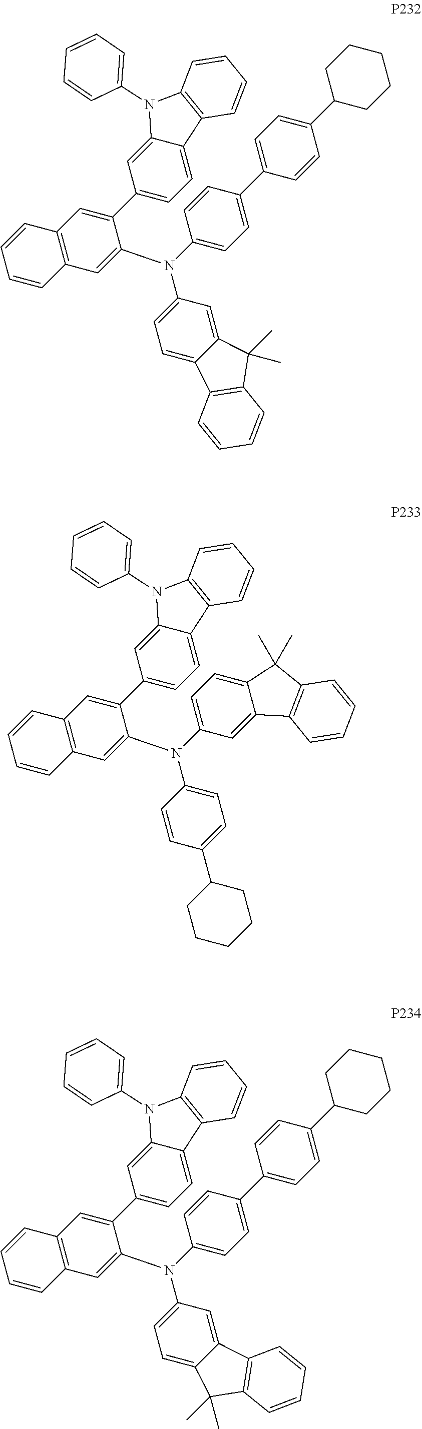 [SMILES: CC1(C)c2ccccc2-c2ccc(N(c3ccc(-c4ccc(C5CCCCC5)cc4)cc3)c3cc4ccccc4cc3-c3ccc4c5ccccc5n(-c5ccccc5)c4c3)cc21 ; CC1(C)c2ccccc2-c2cc(N(c3ccc(C4CCCCC4)cc3)c3cc4ccccc4cc3-c3ccc4c5ccccc5n(-c5ccccc5)c4c3)ccc21 ; CC1(C)c2ccccc2-c2cc(N(c3ccc(-c4ccc(C5CCCCC5)cc4)cc3)c3cc4ccccc4cc3-c3ccc4c5ccccc5n(-c5ccccc5)c4c3)ccc21]

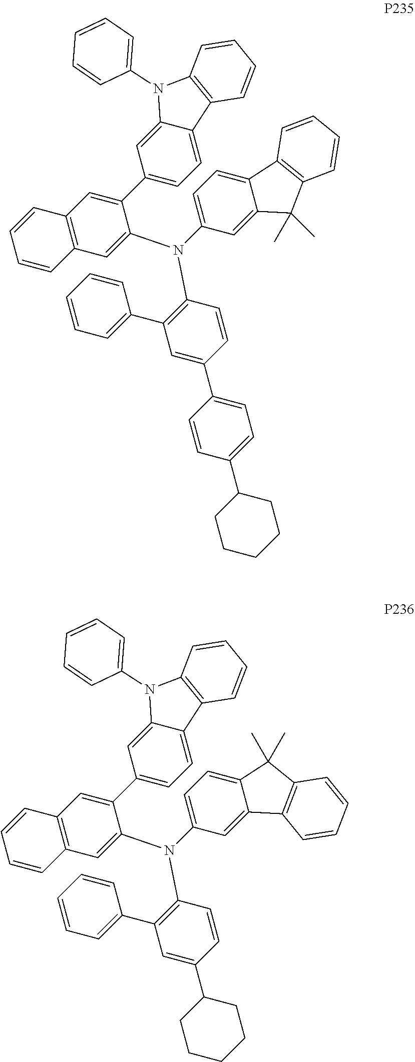 [SMILES: CC1(C)c2ccccc2-c2ccc(N(c3ccc(-c4ccc(C5CCCCC5)cc4)cc3-c3ccccc3)c3cc4ccccc4cc3-c3ccc4c5ccccc5n(-c5ccccc5)c4c3)cc21 ; CC1(C)c2ccccc2-c2cc(N(c3ccc(C4CCCCC4)cc3-c3ccccc3)c3cc4ccccc4cc3-c3ccc4c5ccccc5n(-c5ccccc5)c4c3)ccc21]

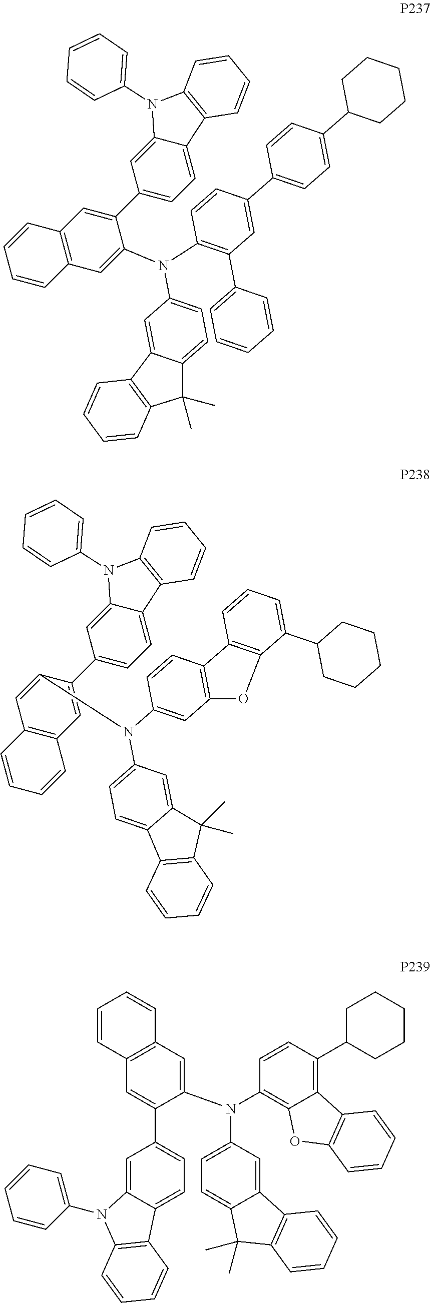 [SMILES: CC1(C)c2ccccc2-c2cc(N(c3ccc(-c4ccc(C5CCCCC5)cc4)cc3-c3ccccc3)c3cc4ccccc4cc3-c3ccc4c5ccccc5n(-c5ccccc5)c4c3)ccc21 ; CC1(C)c2ccccc2-c2ccc(N(c3ccc4c(c3)oc3c(C5CCCCC5)cccc34)c3cc4ccccc4cc3-c3ccc4c5ccccc5n(-c5ccccc5)c4c3)cc21 ; CC1(C)c2ccccc2-c2cc(N(c3cc4ccccc4cc3-c3ccc4c5ccccc5n(-c5ccccc5)c4c3)c3ccc(C4CCCCC4)c4c3oc3ccccc34)ccc21]

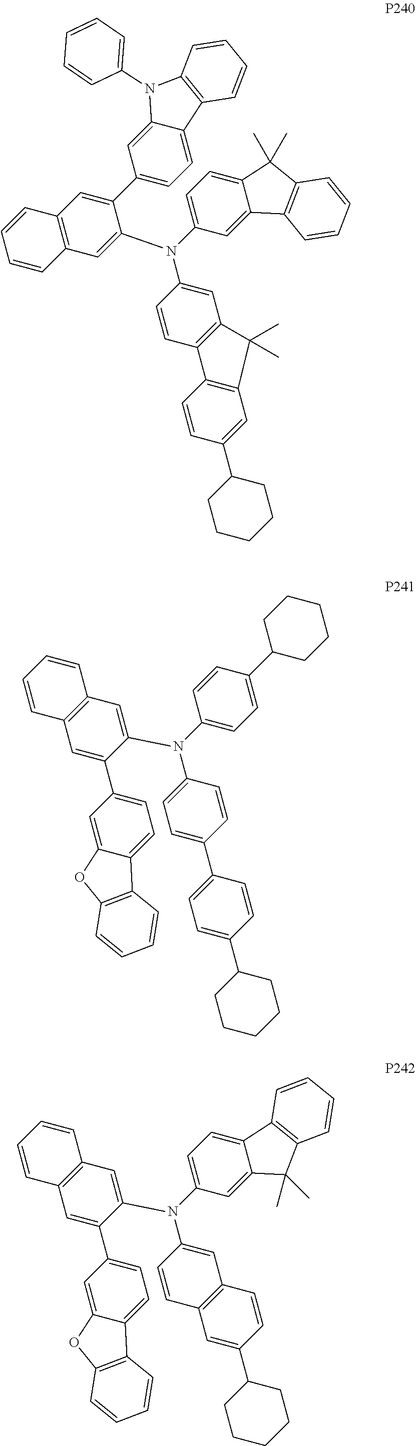 [SMILES: CC1(C)c2ccccc2-c2cc(N(c3ccc4c(c3)C(C)(C)c3cc(C5CCCCC5)ccc3-4)c3cc4ccccc4cc3-c3ccc4c5ccccc5n(-c5ccccc5)c4c3)ccc21 ; c1ccc2cc(N(c3ccc(-c4ccc(C5CCCCC5)cc4)cc3)c3ccc(C4CCCCC4)cc3)c(-c3ccc4c(c3)oc3ccccc34)cc2c1 ; CC1(C)c2ccccc2-c2ccc(N(c3ccc4cc(C5CCCCC5)ccc4c3)c3cc4ccccc4cc3-c3ccc4c(c3)oc3ccccc34)cc21]

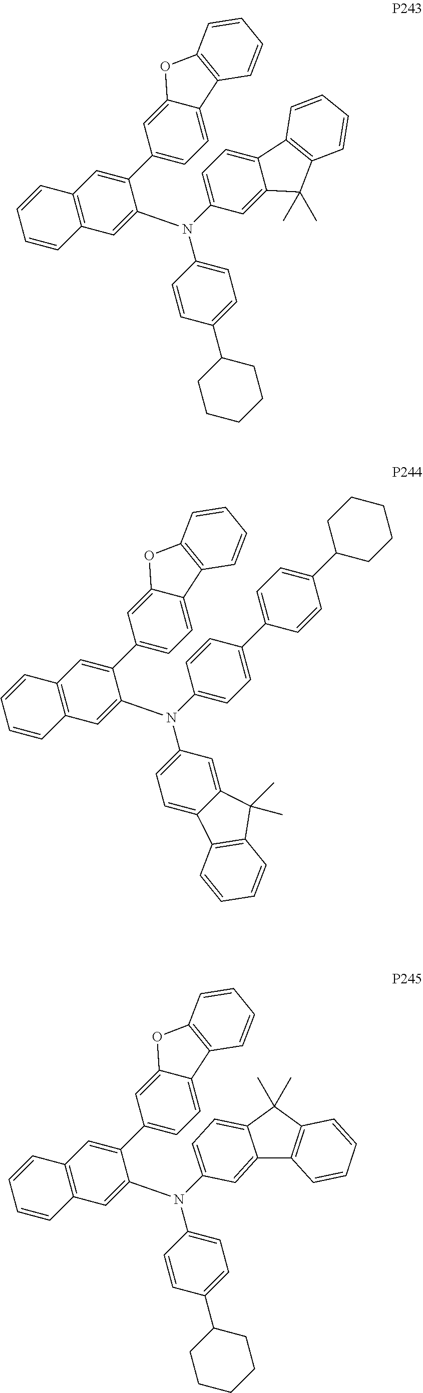 [SMILES: CC1(C)c2ccccc2-c2ccc(N(c3ccc(C4CCCCC4)cc3)c3cc4ccccc4cc3-c3ccc4c(c3)oc3ccccc34)cc21 ; CC1(C)c2ccccc2-c2ccc(N(c3ccc(-c4ccc(C5CCCCC5)cc4)cc3)c3cc4ccccc4cc3-c3ccc4c(c3)oc3ccccc34)cc21 ; CC1(C)c2ccccc2-c2cc(N(c3ccc(C4CCCCC4)cc3)c3cc4ccccc4cc3-c3ccc4c(c3)oc3ccccc34)ccc21]

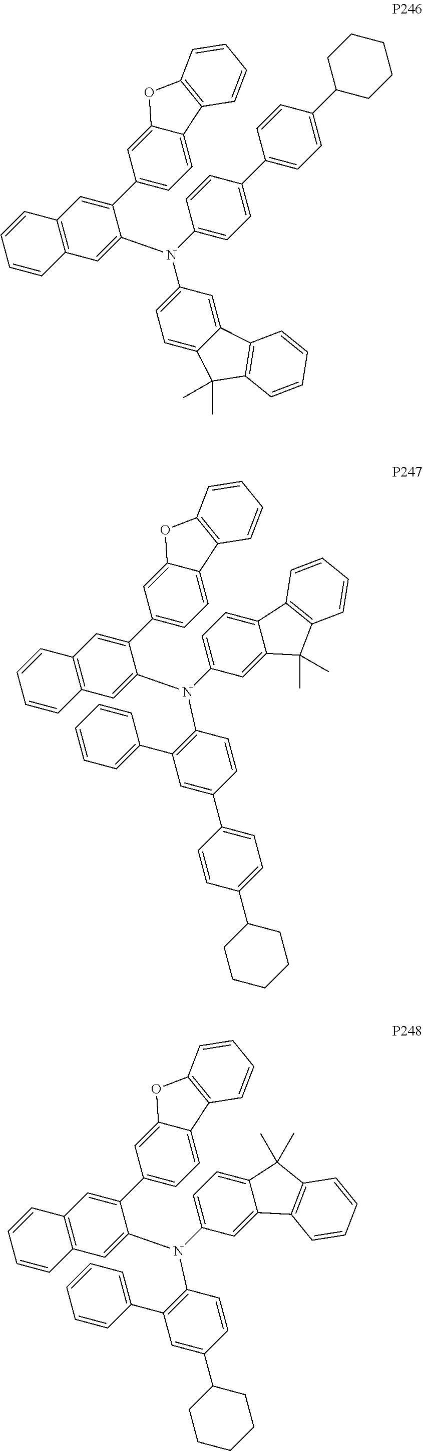 [SMILES: CC1(C)c2ccccc2-c2cc(N(c3ccc(-c4ccc(C5CCCCC5)cc4)cc3)c3cc4ccccc4cc3-c3ccc4c(c3)oc3ccccc34)ccc21 ; CC1(C)c2ccccc2-c2ccc(N(c3ccc(-c4ccc(C5CCCCC5)cc4)cc3-c3ccccc3)c3cc4ccccc4cc3-c3ccc4c(c3)oc3ccccc34)cc21 ; CC1(C)c2ccccc2-c2cc(N(c3ccc(C4CCCCC4)cc3-c3ccccc3)c3cc4ccccc4cc3-c3ccc4c(c3)oc3ccccc34)ccc21]

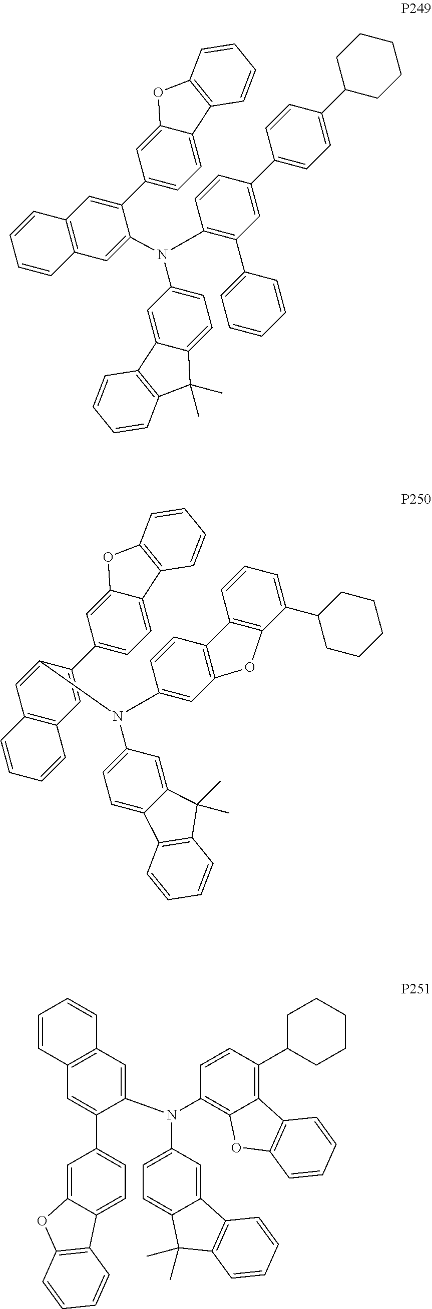 [SMILES: CC1(C)c2ccccc2-c2cc(N(c3ccc(-c4ccc(C5CCCCC5)cc4)cc3-c3ccccc3)c3cc4ccccc4cc3-c3ccc4c(c3)oc3ccccc34)ccc21 ; CC1(C)c2ccccc2-c2ccc(N(c3ccc4c(c3)oc3c(C5CCCCC5)cccc34)c3cc4ccccc4cc3-c3ccc4c(c3)oc3ccccc34)cc21 ; CC1(C)c2ccccc2-c2cc(N(c3cc4ccccc4cc3-c3ccc4c(c3)oc3ccccc34)c3ccc(C4CCCCC4)c4c3oc3ccccc34)ccc21]

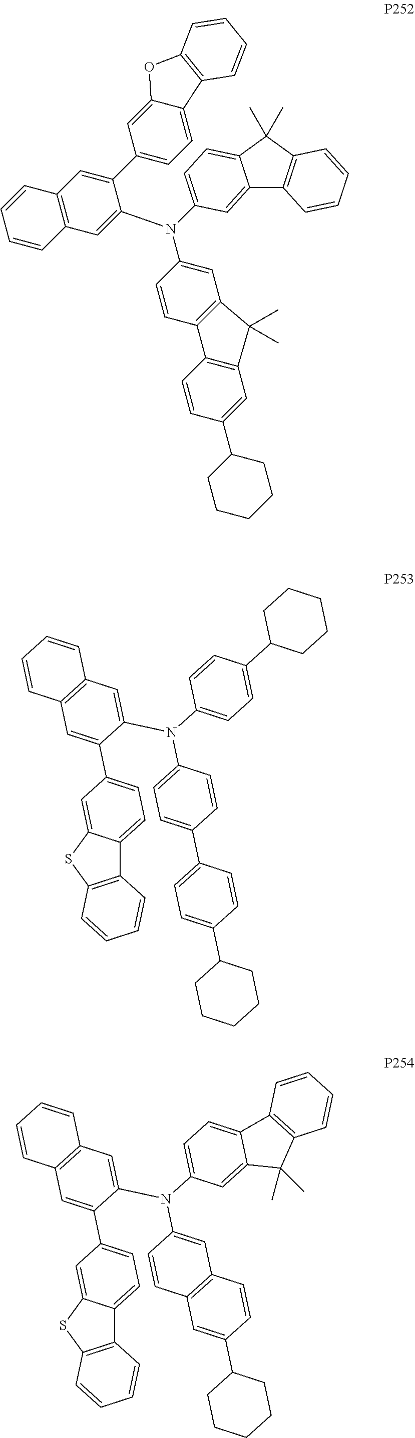 [SMILES: CC1(C)c2ccccc2-c2cc(N(c3ccc4c(c3)C(C)(C)c3cc(C5CCCCC5)ccc3-4)c3cc4ccccc4cc3-c3ccc4c(c3)oc3ccccc34)ccc21 ; c1ccc2cc(N(c3ccc(-c4ccc(C5CCCCC5)cc4)cc3)c3ccc(C4CCCCC4)cc3)c(-c3ccc4c(c3)sc3ccccc34)cc2c1 ; CC1(C)c2ccccc2-c2ccc(N(c3ccc4cc(C5CCCCC5)ccc4c3)c3cc4ccccc4cc3-c3ccc4c(c3)sc3ccccc34)cc21]

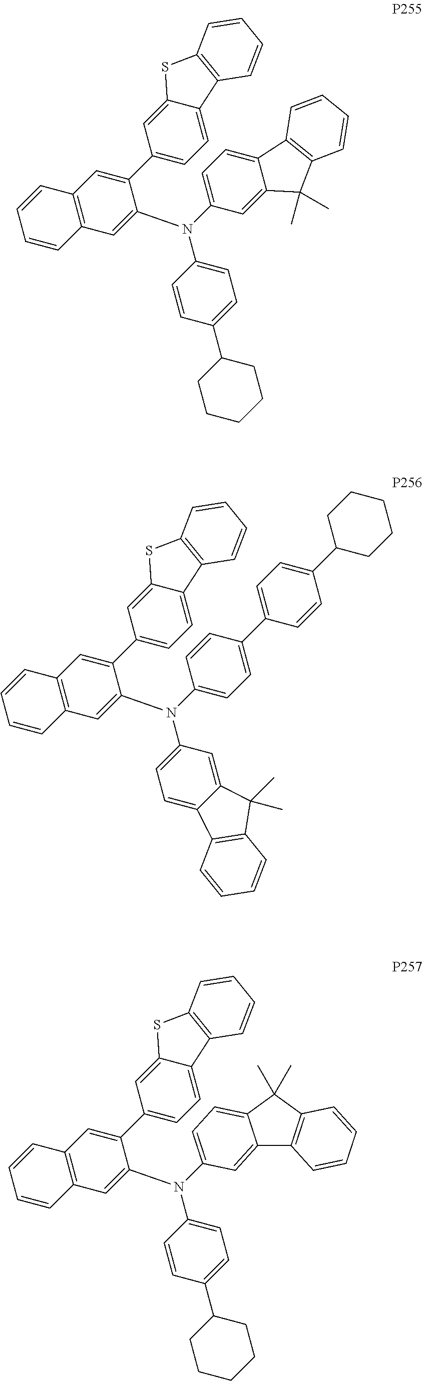 [SMILES: CC1(C)c2ccccc2-c2ccc(N(c3ccc(C4CCCCC4)cc3)c3cc4ccccc4cc3-c3ccc4c(c3)sc3ccccc34)cc21 ; CC1(C)c2ccccc2-c2ccc(N(c3ccc(-c4ccc(C5CCCCC5)cc4)cc3)c3cc4ccccc4cc3-c3ccc4c(c3)sc3ccccc34)cc21 ; CC1(C)c2ccccc2-c2cc(N(c3ccc(C4CCCCC4)cc3)c3cc4ccccc4cc3-c3ccc4c(c3)sc3ccccc34)ccc21]

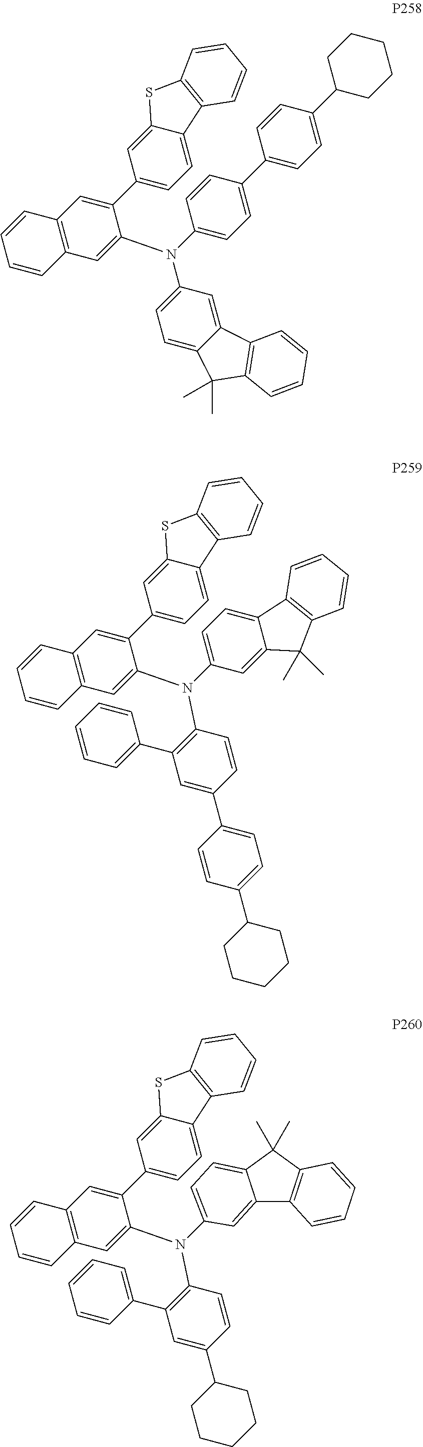 [SMILES: CC1(C)c2ccccc2-c2cc(N(c3ccc(-c4ccc(C5CCCCC5)cc4)cc3)c3cc4ccccc4cc3-c3ccc4c(c3)sc3ccccc34)ccc21 ; CC1(C)c2ccccc2-c2ccc(N(c3ccc(-c4ccc(C5CCCCC5)cc4)cc3-c3ccccc3)c3cc4ccccc4cc3-c3ccc4c(c3)sc3ccccc34)cc21 ; CC1(C)c2ccccc2-c2cc(N(c3ccc(C4CCCCC4)cc3-c3ccccc3)c3cc4ccccc4cc3-c3ccc4c(c3)sc3ccccc34)ccc21]

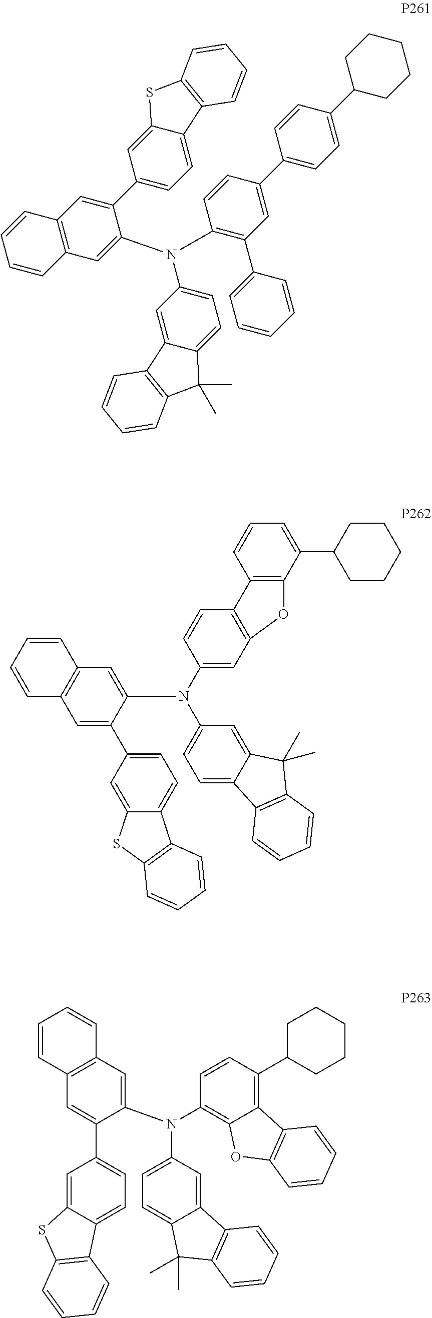 [SMILES: CC1(C)c2ccccc2-c2cc(N(c3ccc(-c4ccc(C5CCCCC5)cc4)cc3-c3ccccc3)c3cc4ccccc4cc3-c3ccc4c(c3)sc3ccccc34)ccc21 ; CC1(C)c2ccccc2-c2ccc(N(c3ccc4c(c3)oc3c(C5CCCCC5)cccc34)c3cc4ccccc4cc3-c3ccc4c(c3)sc3ccccc34)cc21 ; CC1(C)c2ccccc2-c2cc(N(c3cc4ccccc4cc3-c3ccc4c(c3)sc3ccccc34)c3ccc(C4CCCCC4)c4c3oc3ccccc34)ccc21]

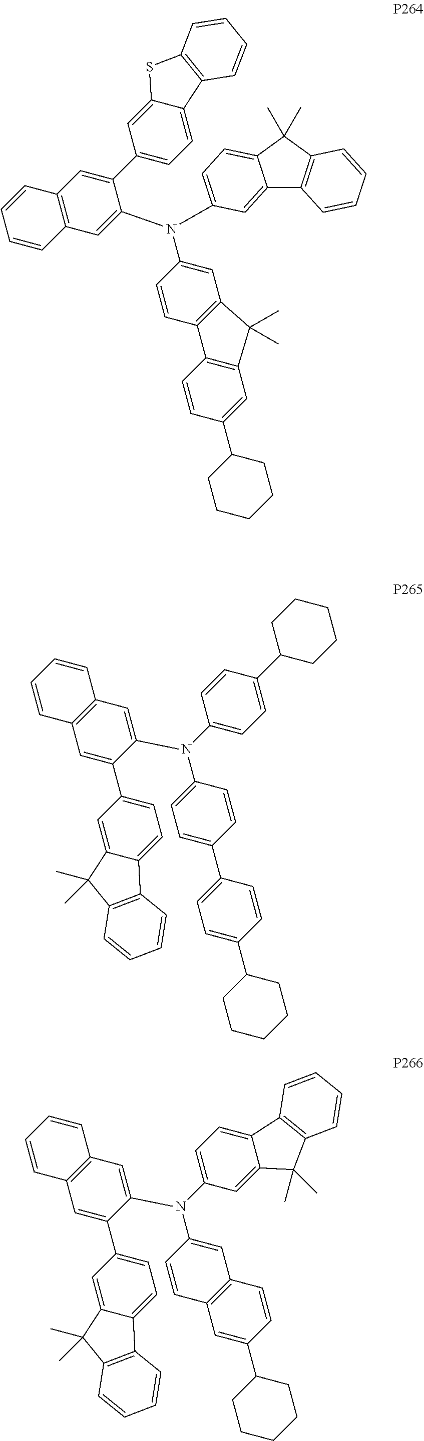 [SMILES: CC1(C)c2ccccc2-c2cc(N(c3ccc4c(c3)C(C)(C)c3cc(C5CCCCC5)ccc3-4)c3cc4ccccc4cc3-c3ccc4c(c3)sc3ccccc34)ccc21 ; CC1(C)c2ccccc2-c2ccc(-c3cc4ccccc4cc3N(c3ccc(-c4ccc(C5CCCCC5)cc4)cc3)c3ccc(C4CCCCC4)cc3)cc21 ; CC1(C)c2ccccc2-c2ccc(-c3cc4ccccc4cc3N(c3ccc4c(c3)C(C)(C)c3ccccc3-4)c3ccc4cc(C5CCCCC5)ccc4c3)cc21]

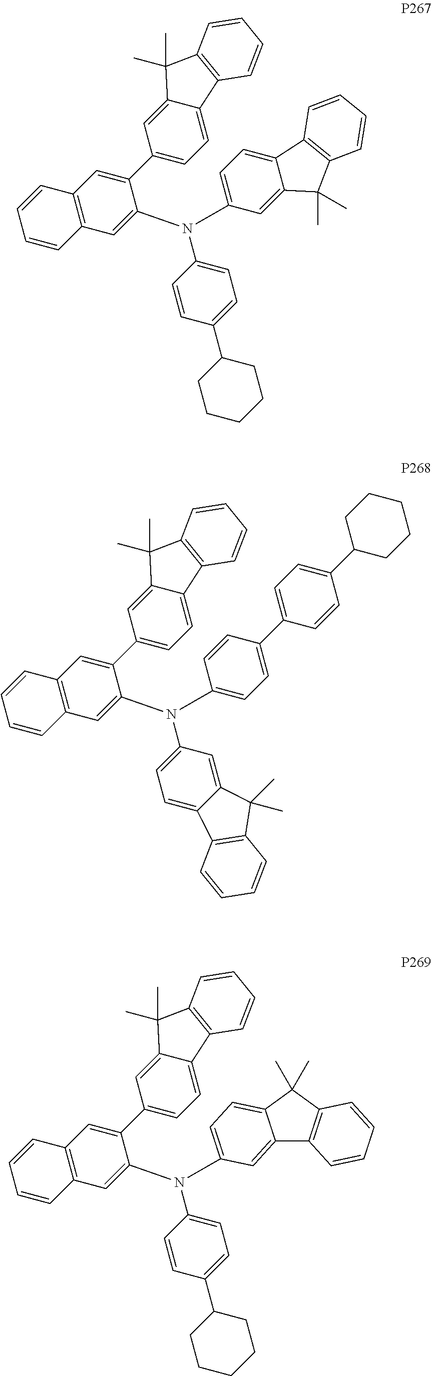 [SMILES: CC1(C)c2ccccc2-c2ccc(-c3cc4ccccc4cc3N(c3ccc(C4CCCCC4)cc3)c3ccc4c(c3)C(C)(C)c3ccccc3-4)cc21 ; CC1(C)c2ccccc2-c2ccc(-c3cc4ccccc4cc3N(c3ccc(-c4ccc(C5CCCCC5)cc4)cc3)c3ccc4c(c3)C(C)(C)c3ccccc3-4)cc21 ; CC1(C)c2ccccc2-c2cc(N(c3ccc(C4CCCCC4)cc3)c3cc4ccccc4cc3-c3ccc4c(c3)C(C)(C)c3ccccc3-4)ccc21]

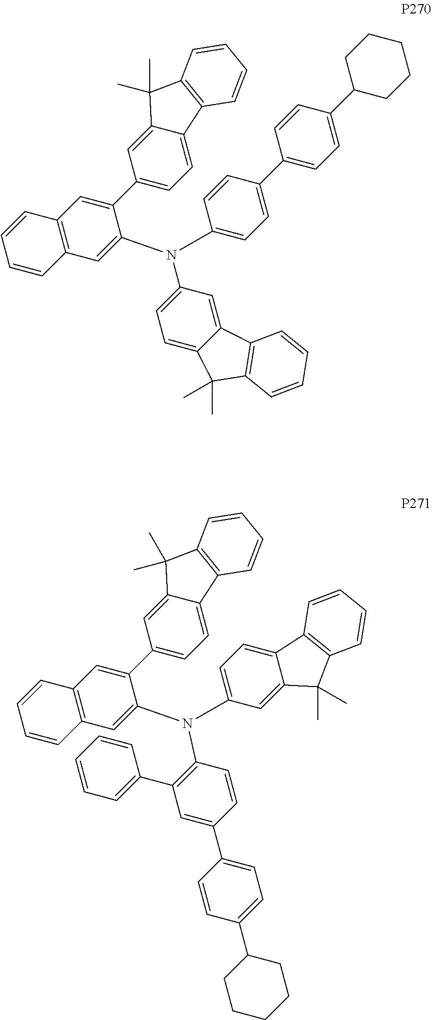 [SMILES: CC1(C)c2ccccc2-c2cc(N(c3ccc(-c4ccc(C5CCCCC5)cc4)cc3)c3cc4ccccc4cc3-c3ccc4c(c3)C(C)(C)c3ccccc3-4)ccc21 ; CC1(C)c2ccccc2-c2ccc(-c3cc4ccccc4cc3N(c3ccc4c(c3)C(C)(C)c3ccccc3-4)c3ccc(-c4ccc(C5CCCCC5)cc4)cc3-c3ccccc3)cc21]

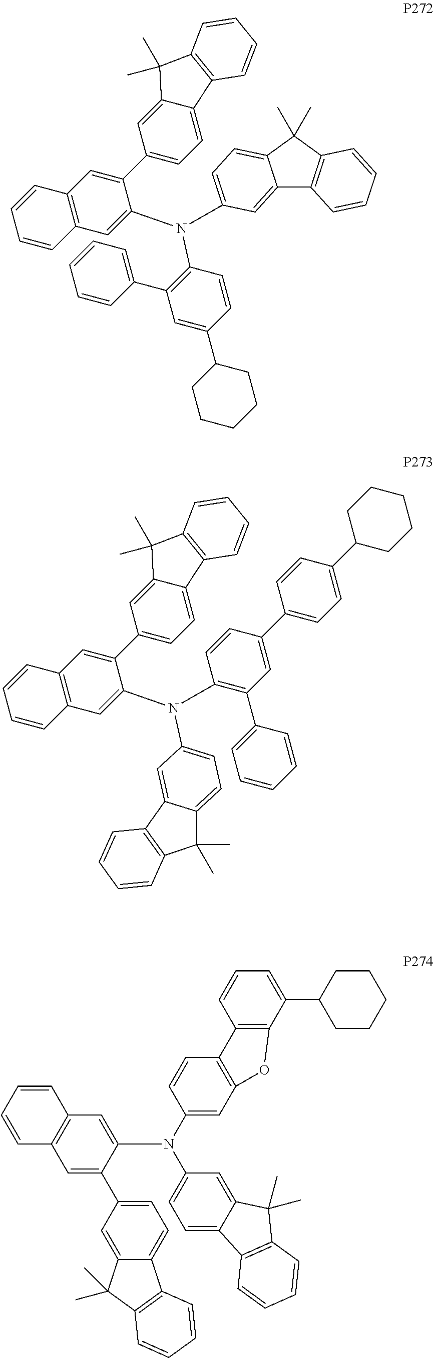 [SMILES: CC1(C)c2ccccc2-c2cc(N(c3ccc(C4CCCCC4)cc3-c3ccccc3)c3cc4ccccc4cc3-c3ccc4c(c3)C(C)(C)c3ccccc3-4)ccc21 ; CC1(C)c2ccccc2-c2cc(N(c3ccc(-c4ccc(C5CCCCC5)cc4)cc3-c3ccccc3)c3cc4ccccc4cc3-c3ccc4c(c3)C(C)(C)c3ccccc3-4)ccc21 ; CC1(C)c2ccccc2-c2ccc(-c3cc4ccccc4cc3N(c3ccc4c(c3)C(C)(C)c3ccccc3-4)c3ccc4c(c3)oc3c(C5CCCCC5)cccc34)cc21]

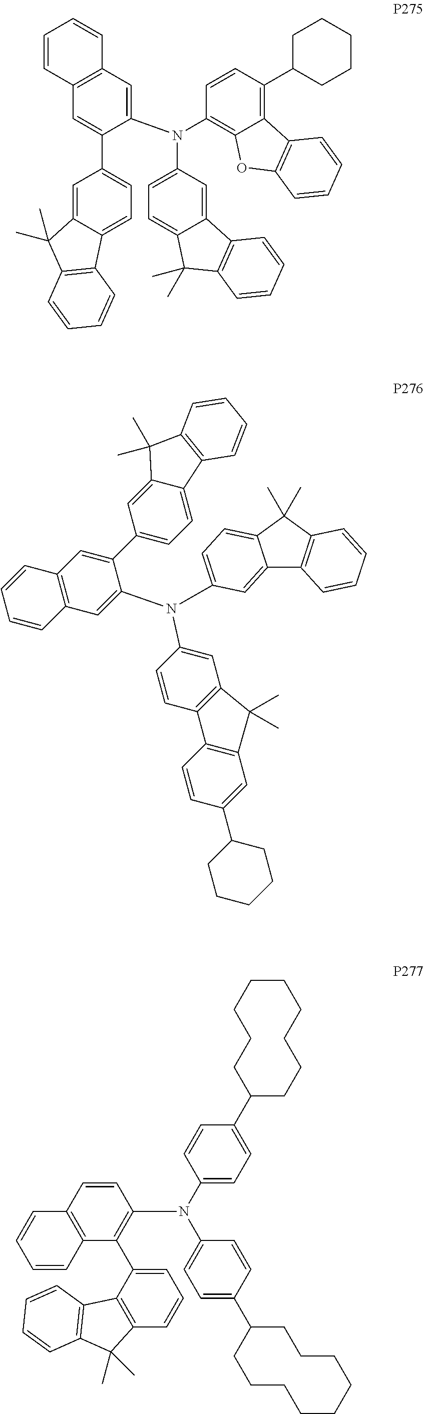 [SMILES: CC1(C)c2ccccc2-c2cc(N(c3cc4ccccc4cc3-c3ccc4c(c3)C(C)(C)c3ccccc3-4)c3ccc(C4CCCCC4)c4c3oc3ccccc34)ccc21 ; CC1(C)c2ccccc2-c2cc(N(c3ccc4c(c3)C(C)(C)c3cc(C5CCCCC5)ccc3-4)c3cc4ccccc4cc3-c3ccc4c(c3)C(C)(C)c3ccccc3-4)ccc21 ; CC1(C)c2ccccc2-c2c(-c3c(N(c4ccc(C5CCCCCCCCC5)cc4)c4ccc(C5CCCCCCCCC5)cc4)ccc4ccccc34)cccc21]

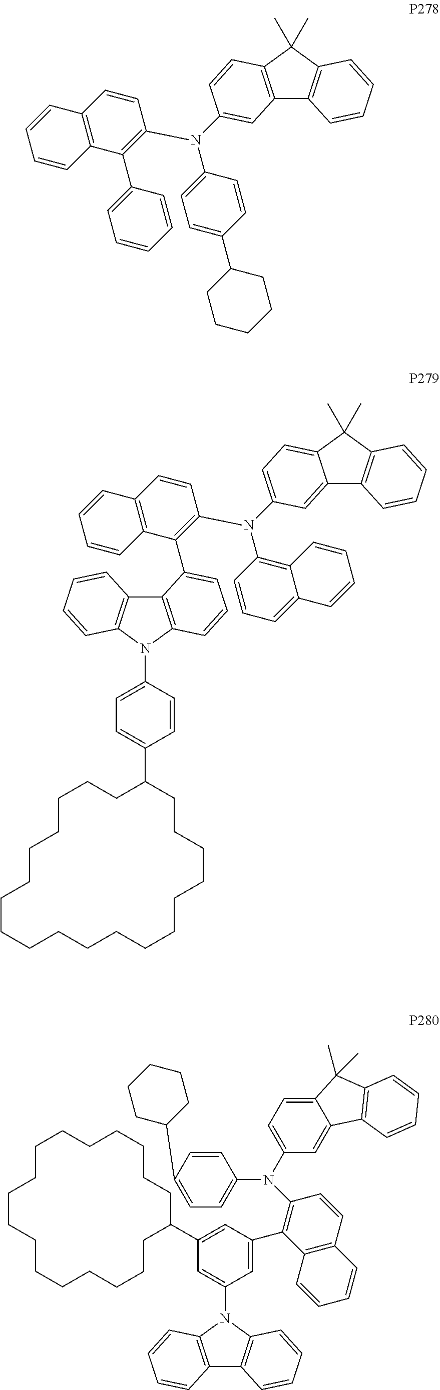 [SMILES: CC1(C)c2ccccc2-c2cc(N(c3ccc(C4CCCCC4)cc3)c3ccc4ccccc4c3-c3ccccc3)ccc21 ; CC1(C)c2ccccc2-c2cc(N(c3ccc4ccccc4c3-c3cccc4c3c3ccccc3n4-c3ccc(C4CCCCCCCCCCCCCCCCCCC4)cc3)c3cccc4ccccc34)ccc21 ; CC1(C)c2ccccc2-c2cc(N(c3ccc(C4CCCCC4)cc3)c3ccc4ccccc4c3-c3cc(C4CCCCCCCCCCCCCCCCC4)cc(-n4c5ccccc5c5ccccc54)c3)ccc21]

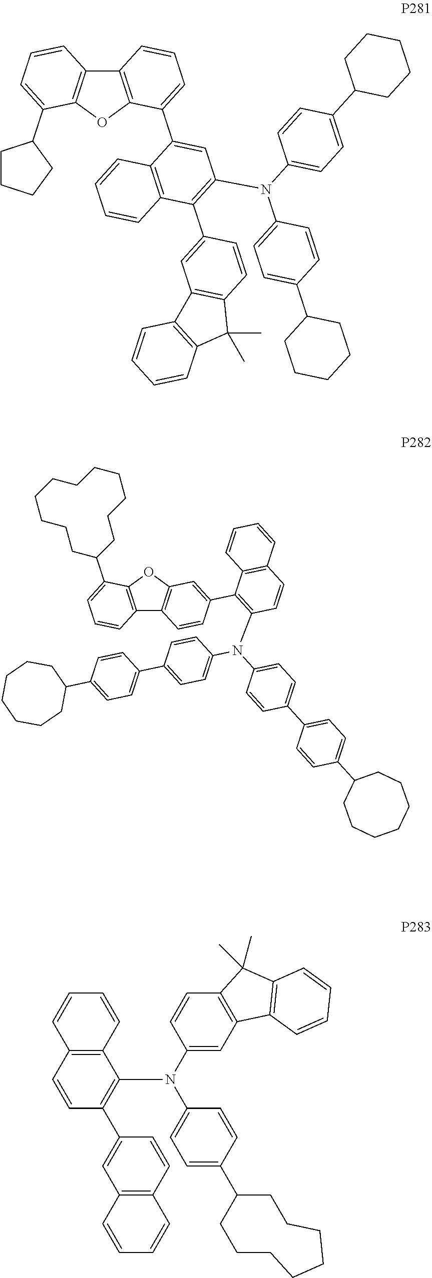 [SMILES: CC1(C)c2ccccc2-c2cc(-c3c(N(c4ccc(C5CCCCC5)cc4)c4ccc(C5CCCCC5)cc4)cc(-c4cccc5c4oc4c(C6CCCC6)cccc45)c4ccccc34)ccc21 ; c1ccc2c(-c3ccc4c(c3)oc3c(C5CCCCCCCCCCC5)cccc34)c(N(c3ccc(-c4ccc(C5CCCCCCC5)cc4)cc3)c3ccc(-c4ccc(C5CCCCCCC5)cc4)cc3)ccc2c1 ; CC1(C)c2ccccc2-c2cc(N(c3ccc(C4CCCCCCCC4)cc3)c3c(-c4ccc5ccccc5c4)ccc4ccccc34)ccc21]

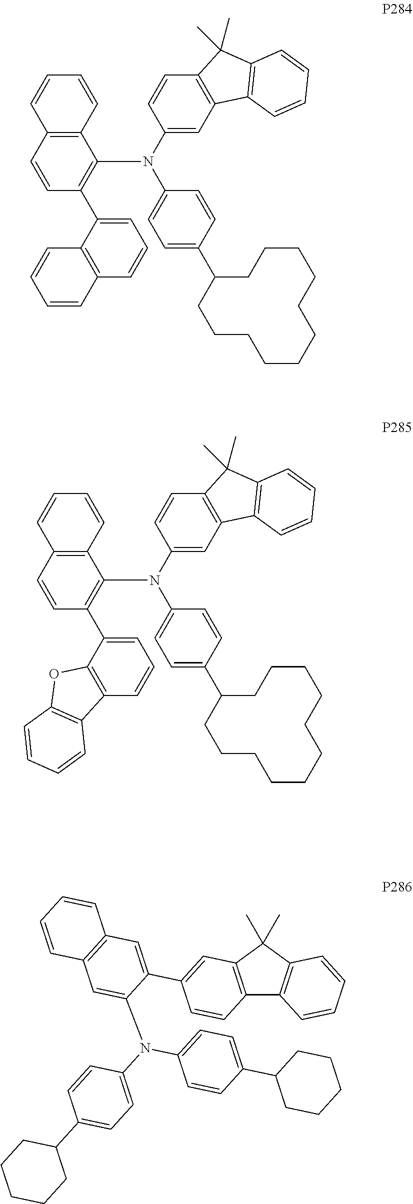 [SMILES: CC1(C)c2ccccc2-c2cc(N(c3ccc(C4CCCCCCCCCCC4)cc3)c3c(-c4cccc5ccccc45)ccc4ccccc34)ccc21 ; CC1(C)c2ccccc2-c2cc(N(c3ccc(C4CCCCCCCCCCC4)cc3)c3c(-c4cccc5c4oc4ccccc45)ccc4ccccc34)ccc21 ; CC1(C)c2ccccc2-c2ccc(-c3cc4ccccc4cc3N(c3ccc(C4CCCCC4)cc3)c3ccc(C4CCCCC4)cc3)cc21]

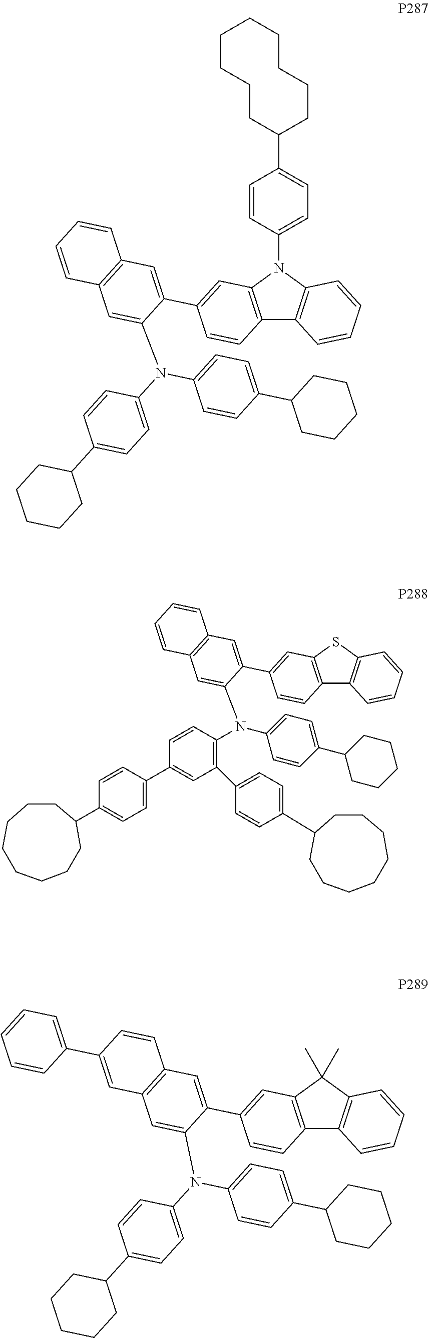 [SMILES: c1ccc2cc(N(c3ccc(C4CCCCC4)cc3)c3ccc(C4CCCCC4)cc3)c(-c3ccc4c5ccccc5n(-c5ccc(C6CCCCCCCCC6)cc5)c4c3)cc2c1 ; c1ccc2cc(N(c3ccc(C4CCCCC4)cc3)c3ccc(-c4ccc(C5CCCCCCCC5)cc4)cc3-c3ccc(C4CCCCCCCC4)cc3)c(-c3ccc4c(c3)sc3ccccc34)cc2c1 ; CC1(C)c2ccccc2-c2ccc(-c3cc4ccc(-c5ccccc5)cc4cc3N(c3ccc(C4CCCCC4)cc3)c3ccc(C4CCCCC4)cc3)cc21]

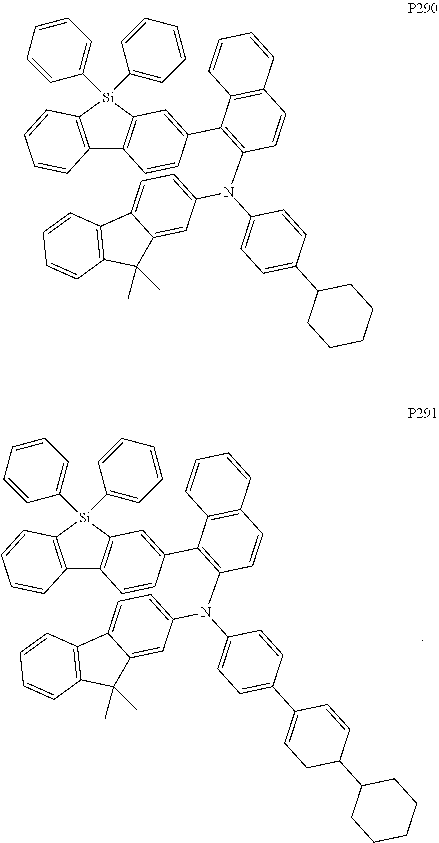 [SMILES: CC1(C)c2ccccc2-c2ccc(N(c3ccc(C4CCCCC4)cc3)c3ccc4ccccc4c3-c3ccc4c(c3)[Si](c3ccccc3)(c3ccccc3)c3ccccc3-4)cc21 ; CC1(C)c2ccccc2-c2ccc(N(c3ccc(C4=CCC(C5CCCCC5)C=C4)cc3)c3ccc4ccccc4c3-c3ccc4c(c3)[Si](c3ccccc3)(c3ccccc3)c3ccccc3-4)cc21]

The present invention provides an application of the above compound in an organic electron device, preferably, the above organic compound is particularly applied in the fields, including but not limited to, organic electroluminescent materials, lighting elements, organic thin film transistors, organic field effect transistors, organic thin film solar cells, information labels, electronic artificial skin sheets, sheet-type scanners, electronic paper or organic EL panels, and more preferably applied in organic electroluminescent materials, especially as a hole-transport material or an electron blocking material of an organic electroluminescent device.

The present invention provides an organic electroluminescent device, including a first electrode, a second electrode, and at least one organic layer inserted between the first electrode and the second organic compounds, where the organic layer contains at least one of the above organic compounds. More specifically, the organic layer may be further divided into a plurality of regions. For example, the organic layer may include a hole transport region, a luminescent layer, an electron transport region and the like.

The present invention further provides an organic electroluminescent device, including an anode layer, a plurality of luminescent functional layers and a cathode layer; the plurality of luminescent functional layers include at least one of a hole injection layer, a hole transport layer, an electron blocking layer, a luminescent layer and an electron transport layer which are successively formed; the hole injection layer is formed on the anode layer, and the anode layer is formed on the electron transport layer, where, the hole transport layer and/or electron blocking layer contains the above organic compound.

### Preferred Embodiment II

The compound of the present invention preferably has a structure as shown in Formula (II):

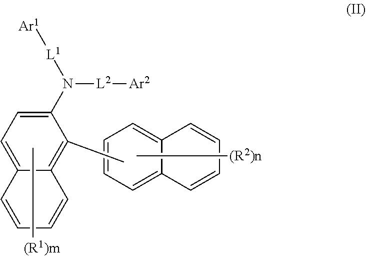

where, L¹ and L² are each independently selected from a single bond, substituted or unsubstituted C₆-C₅₀ alkylene, a substituted or unsubstituted C₃-C₃₀ heteroarylene group;

Ar¹ and Ar² are each independently selected from H, substituted or unsubstituted C₆-C₅₀ aryl, substituted or unsubstituted C₆-C₅₀ fused aryl, substituted or unsubstituted C₃-C₃₀ heteroaryl, substituted or unsubstituted C₃-C₃₀ fused heteroaryl; and when Ar¹ is H, L1 is not a single bond, and when Ar² is H, L² is not a single bond;

R¹ and R² are each independently selected from H, halogen, carbonyl, carboxyl, cyano, amido, C₁-C₂₀ alkyl, C₃-C₂₀ cycloalkyl, C₂-C₁₂ alkenyl, C₂-C₁₂ alkynyl, C₁-C₁₂ alkoxy, substituted or unsubstituted C₆-C₅₀ aryl, substituted or unsubstituted C₃-C₃₀ heteroaryl, C₆-C₅₀ fused aryl; and R¹ and R² are linked on the naphthalene ring in a single bond way;

m is an integer of 0-6, and n is an integer of 0-7;

when the above groups have a substituent, the substituent is each independently selected from one or more of halogen, carbonyl, carboxyl, cyano, amido, C₁-C₁₀ alkyl, C₃-C₁₀ cycloalkyl, C₂-C₁₀ alkenyl, C₁-C₆ alkoxy, C₁-C₆ thioalkoxy, C₆-C₃₀ monocyclic or fused-cyclic aryl, C₃-C₃₀ monocyclic or fused-cyclic heteroaryl.

In the present invention, the 1-position on the naphthalene ring of the compound is linked to another naphthalene ring, and the 2-position on the naphthalene ring is linked to diarylamido. Such a binaphthyl compound is used as a hole-transport material or electron blocking layer material of the organic electroluminescent device, which may further reduce driving voltage, improve luminous efficiency and prolong the service life compared with the prior art.

In the compound of the present invention, the 1-position on the naphthalene ring is linked to another naphthalene ring, and the 2-position is linked to diarylamido. Moreover, other substituents on the two naphthalene rings are not amine or arylamine substituents, that is, R¹ and R² are not amine or arylamine substituents.

In the above compound of the present invention, preferably, Ar¹ and Ar² are independently selected from substituted or unsubstituted C₆-C₅₀ aryl or fused aryl, substituted or unsubstituted C₃-C₃₀ heteroaryl or fused heteroaryl, preferably, L¹ and L² are a single bond, preferably, R¹ and R² are H.

In the above compound of the present invention, more preferably, Ar¹ and Ar² are each independently selected from

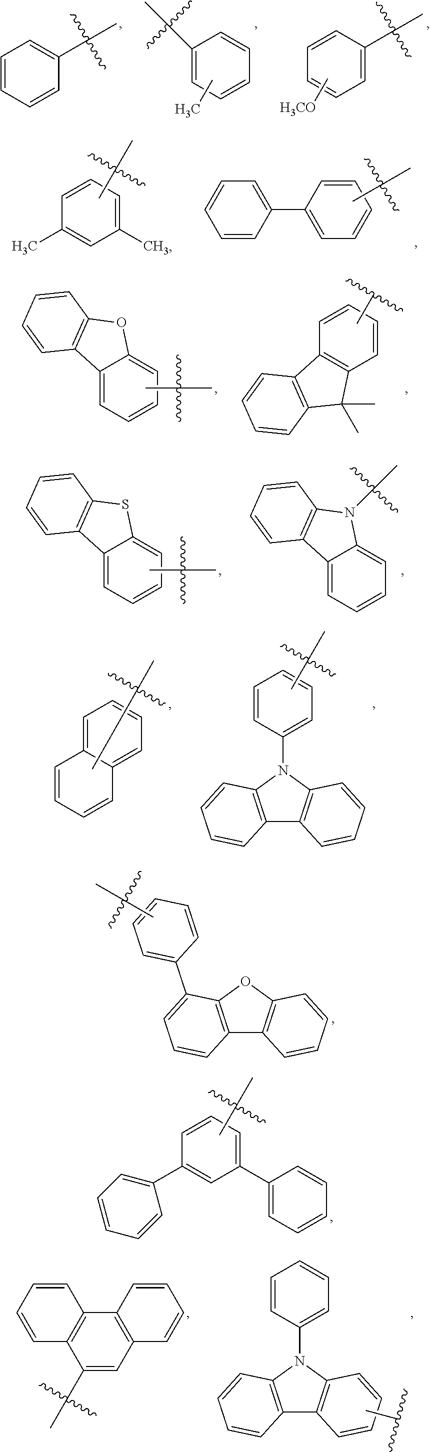

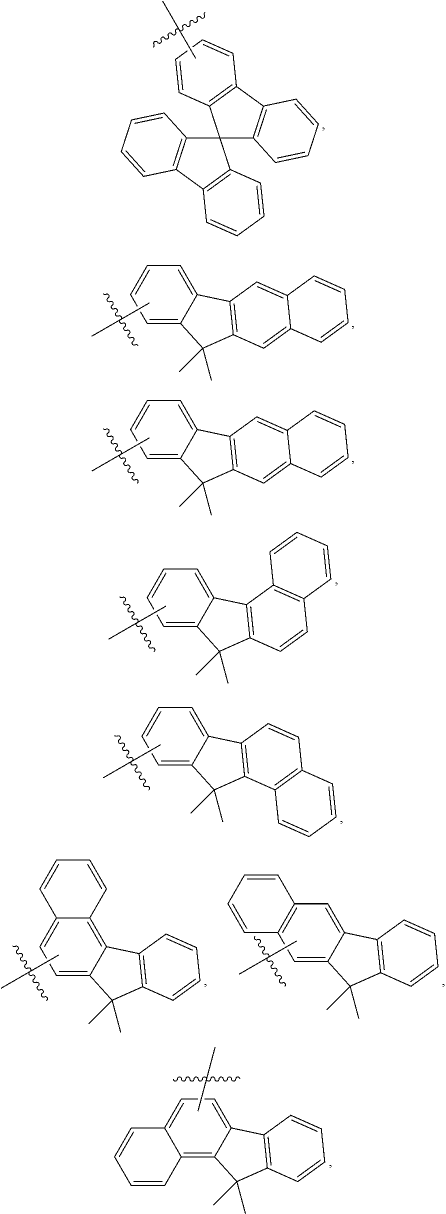

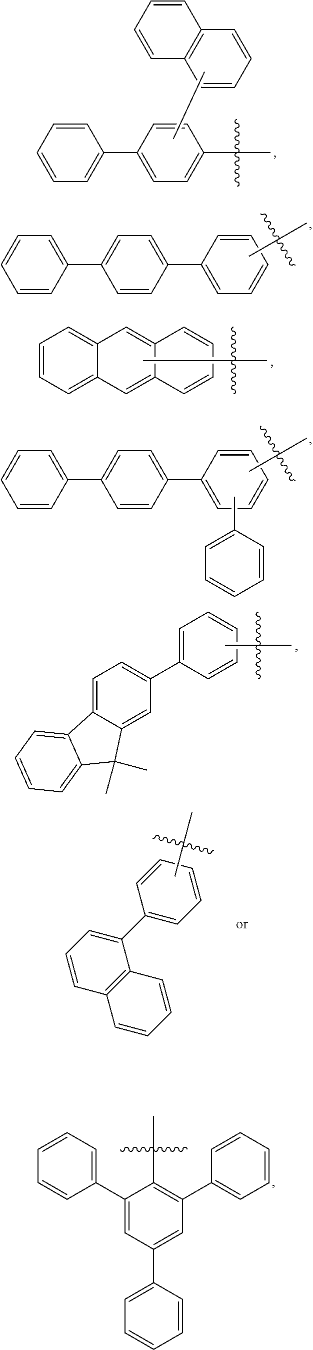

where, [symbol] represents an access position of a group.

The above organic compound of the present invention may be specifically a structure as shown in the following Formula (II-1) or Formula (II-2):

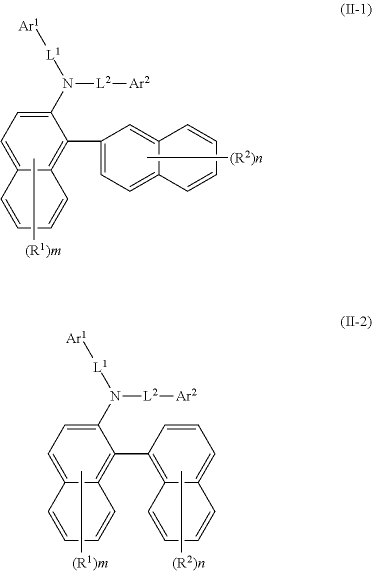

where, L¹, L², Ar¹, Ar², R¹, R², m and n are defined the same as those in the Formula (II).

In the above compound of the present invention, further preferably, Ar¹ and Ar² are each independently selected from the group consisting of substituted or unsubstituted:

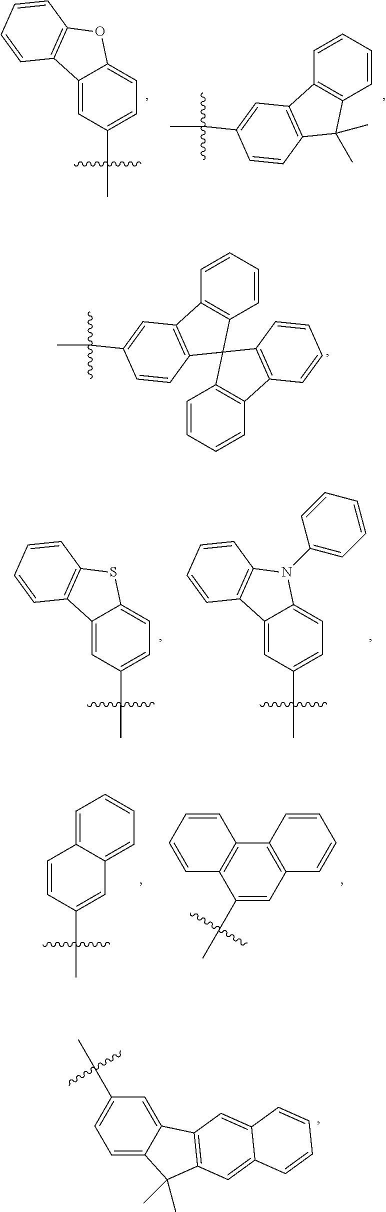

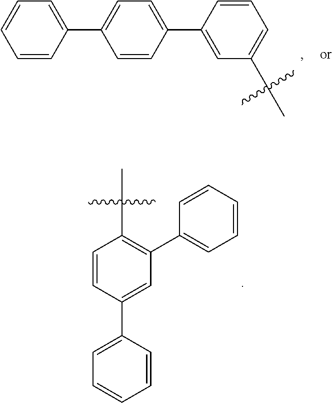 [SMILES: *c1cccc(-c2ccc(-c3ccccc3)cc2)c1 ; *c1ccc(-c2ccccc2)cc1-c1ccccc1]

the compound having the structure as shown in the above Formula (II) of the present invention is preferably any one of the following compounds N1-N419, but these compounds are merely representative.

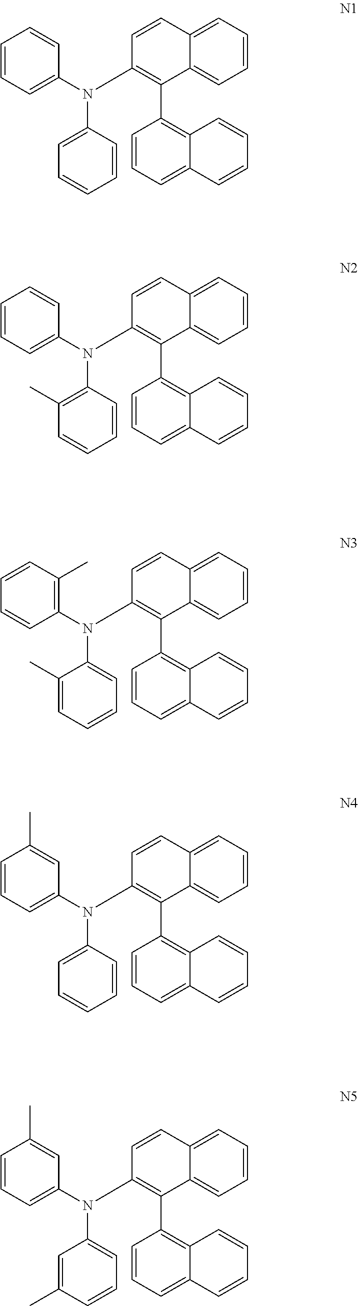 [SMILES: c1ccc(N(c2ccccc2)c2ccc3ccccc3c2-c2cccc3ccccc23)cc1 ; Cc1ccccc1N(c1ccccc1)c1ccc2ccccc2c1-c1cccc2ccccc12 ; Cc1ccccc1N(c1ccccc1C)c1ccc2ccccc2c1-c1cccc2ccccc12 ; Cc1cccc(N(c2ccccc2)c2ccc3ccccc3c2-c2cccc3ccccc23)c1 ; Cc1cccc(N(c2cccc(C)c2)c2ccc3ccccc3c2-c2cccc3ccccc23)c1]

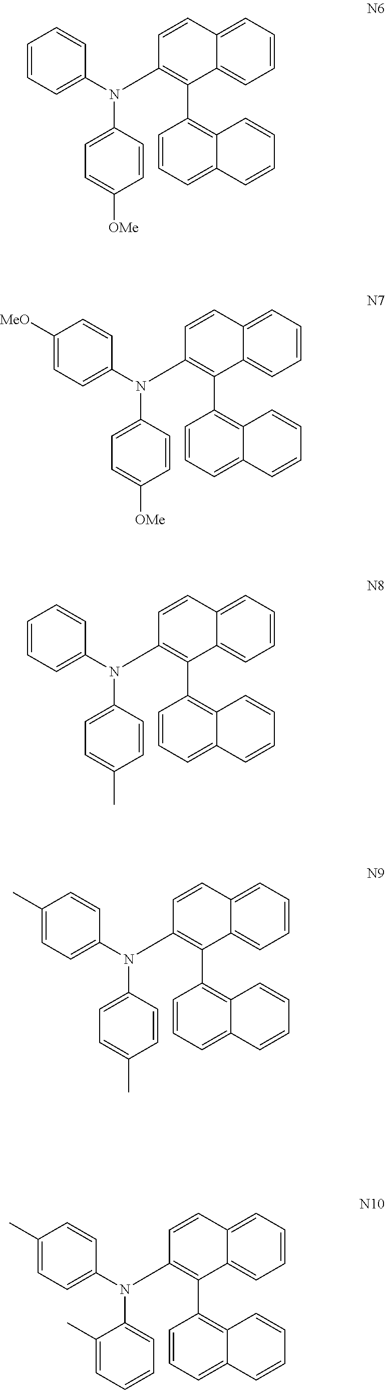 [SMILES: COc1ccc(N(c2ccccc2)c2ccc3ccccc3c2-c2cccc3ccccc23)cc1 ; COc1ccc(N(c2ccc(OC)cc2)c2ccc3ccccc3c2-c2cccc3ccccc23)cc1 ; Cc1ccc(N(c2ccccc2)c2ccc3ccccc3c2-c2cccc3ccccc23)cc1 ; Cc1ccc(N(c2ccc(C)cc2)c2ccc3ccccc3c2-c2cccc3ccccc23)cc1 ; Cc1ccc(N(c2ccccc2C)c2ccc3ccccc3c2-c2cccc3ccccc23)cc1]

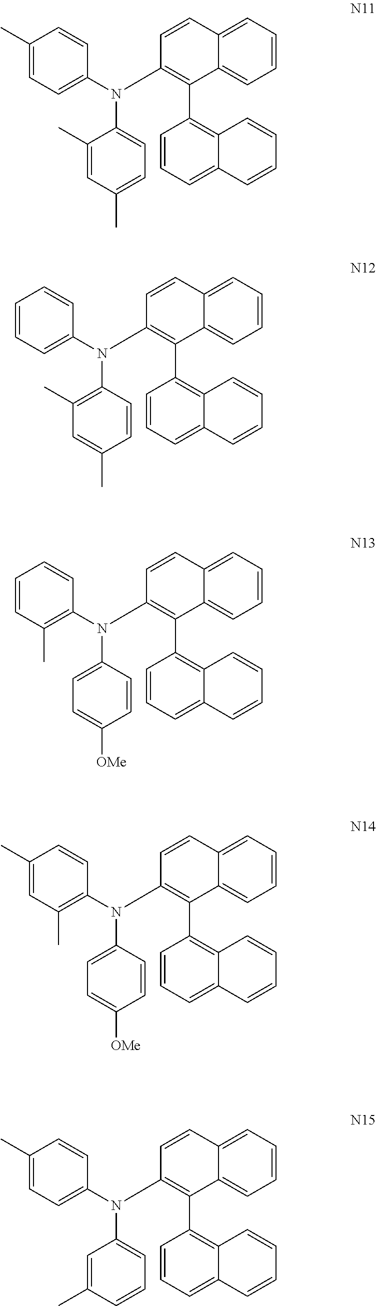 [SMILES: Cc1ccc(N(c2ccc(C)cc2C)c2ccc3ccccc3c2-c2cccc3ccccc23)cc1 ; Cc1ccc(N(c2ccccc2)c2ccc3ccccc3c2-c2cccc3ccccc23)c(C)c1 ; COc1ccc(N(c2ccccc2C)c2ccc3ccccc3c2-c2cccc3ccccc23)cc1 ; COc1ccc(N(c2ccc(C)cc2C)c2ccc3ccccc3c2-c2cccc3ccccc23)cc1 ; Cc1ccc(N(c2cccc(C)c2)c2ccc3ccccc3c2-c2cccc3ccccc23)cc1]

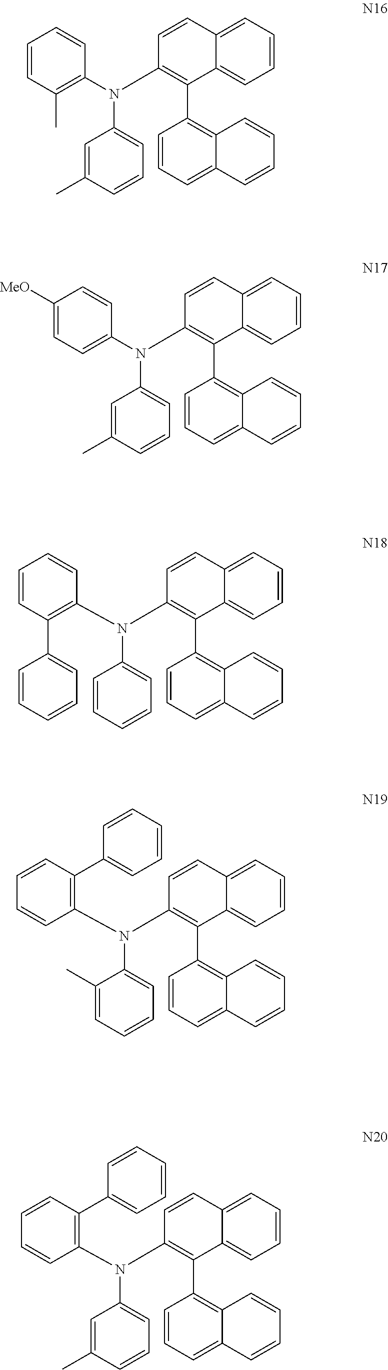 [SMILES: Cc1cccc(N(c2ccccc2C)c2ccc3ccccc3c2-c2cccc3ccccc23)c1 ; COc1ccc(N(c2cccc(C)c2)c2ccc3ccccc3c2-c2cccc3ccccc23)cc1 ; c1ccc(-c2ccccc2N(c2ccccc2)c2ccc3ccccc3c2-c2cccc3ccccc23)cc1 ; Cc1ccccc1N(c1ccccc1-c1ccccc1)c1ccc2ccccc2c1-c1cccc2ccccc12 ; Cc1cccc(N(c2ccccc2-c2ccccc2)c2ccc3ccccc3c2-c2cccc3ccccc23)c1]

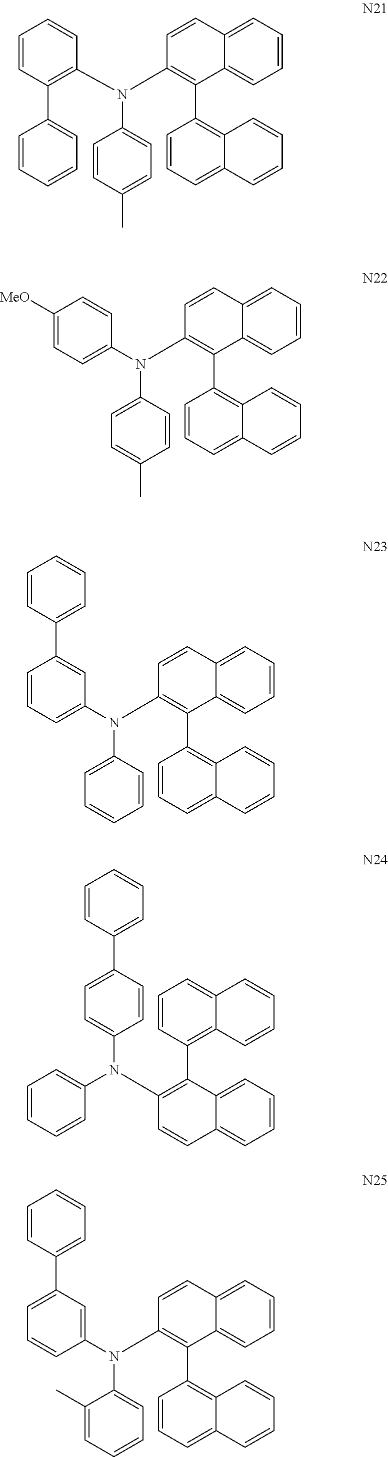 [SMILES: Cc1ccc(N(c2ccccc2-c2ccccc2)c2ccc3ccccc3c2-c2cccc3ccccc23)cc1 ; COc1ccc(N(c2ccc(C)cc2)c2ccc3ccccc3c2-c2cccc3ccccc23)cc1 ; c1ccc(-c2cccc(N(c3ccccc3)c3ccc4ccccc4c3-c3cccc4ccccc34)c2)cc1 ; c1ccc(-c2ccc(N(c3ccccc3)c3ccc4ccccc4c3-c3cccc4ccccc34)cc2)cc1 ; Cc1ccccc1N(c1cccc(-c2ccccc2)c1)c1ccc2ccccc2c1-c1cccc2ccccc12]

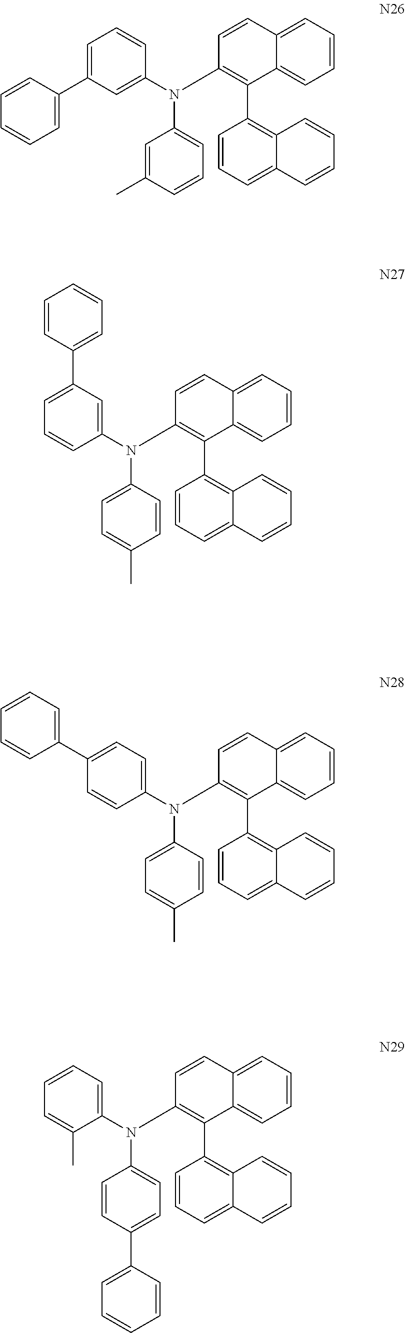 [SMILES: Cc1cccc(N(c2cccc(-c3ccccc3)c2)c2ccc3ccccc3c2-c2cccc3ccccc23)c1 ; Cc1ccc(N(c2cccc(-c3ccccc3)c2)c2ccc3ccccc3c2-c2cccc3ccccc23)cc1 ; Cc1ccc(N(c2ccc(-c3ccccc3)cc2)c2ccc3ccccc3c2-c2cccc3ccccc23)cc1 ; Cc1ccccc1N(c1ccc(-c2ccccc2)cc1)c1ccc2ccccc2c1-c1cccc2ccccc12]

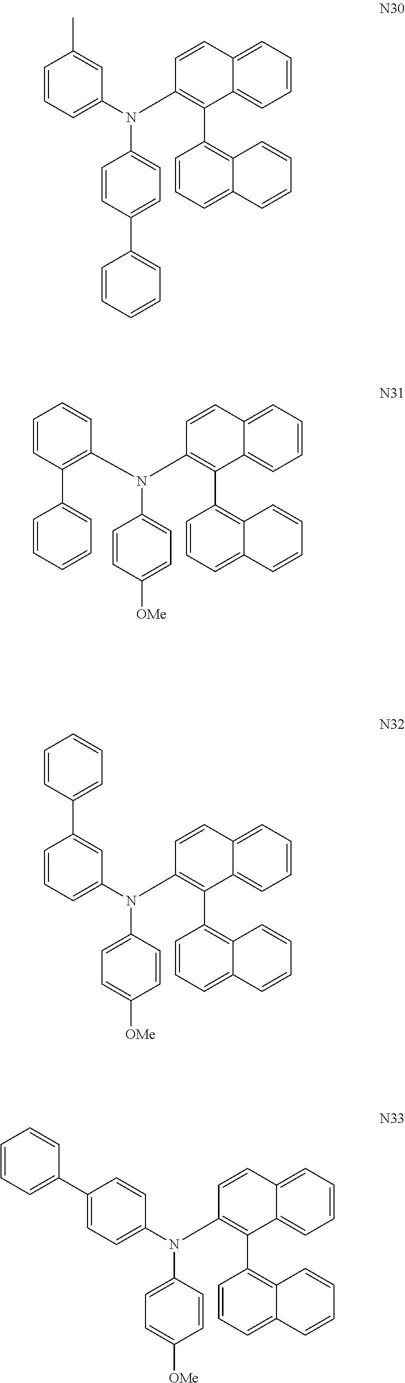 [SMILES: Cc1cccc(N(c2ccc(-c3ccccc3)cc2)c2ccc3ccccc3c2-c2cccc3ccccc23)c1 ; COc1ccc(N(c2ccccc2-c2ccccc2)c2ccc3ccccc3c2-c2cccc3ccccc23)cc1 ; COc1ccc(N(c2cccc(-c3ccccc3)c2)c2ccc3ccccc3c2-c2cccc3ccccc23)cc1 ; COc1ccc(N(c2ccc(-c3ccccc3)cc2)c2ccc3ccccc3c2-c2cccc3ccccc23)cc1]

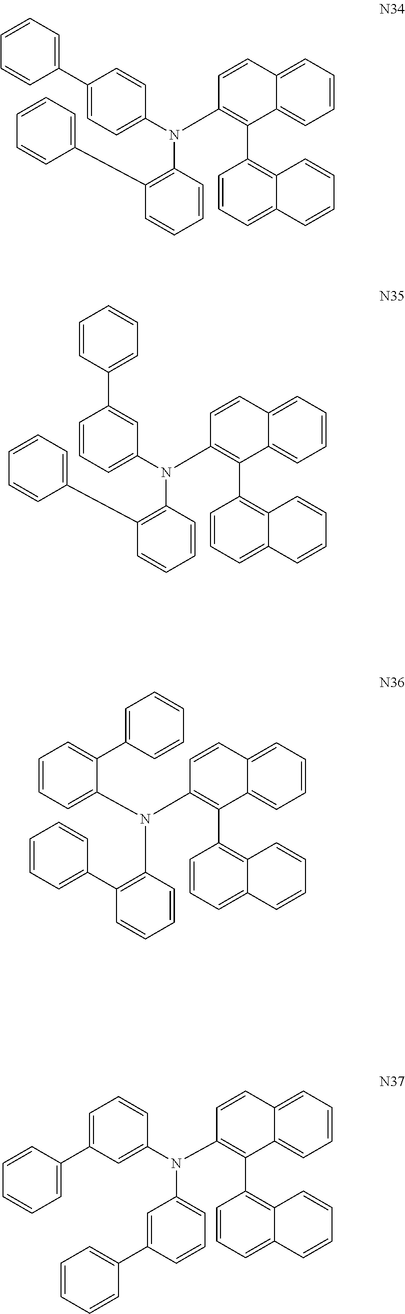 [SMILES: c1ccc(-c2ccc(N(c3ccccc3-c3ccccc3)c3ccc4ccccc4c3-c3cccc4ccccc34)cc2)cc1 ; c1ccc(-c2cccc(N(c3ccccc3-c3ccccc3)c3ccc4ccccc4c3-c3cccc4ccccc34)c2)cc1 ; c1ccc(-c2ccccc2N(c2ccccc2-c2ccccc2)c2ccc3ccccc3c2-c2cccc3ccccc23)cc1 ; c1ccc(-c2cccc(N(c3cccc(-c4ccccc4)c3)c3ccc4ccccc4c3-c3cccc4ccccc34)c2)cc1]

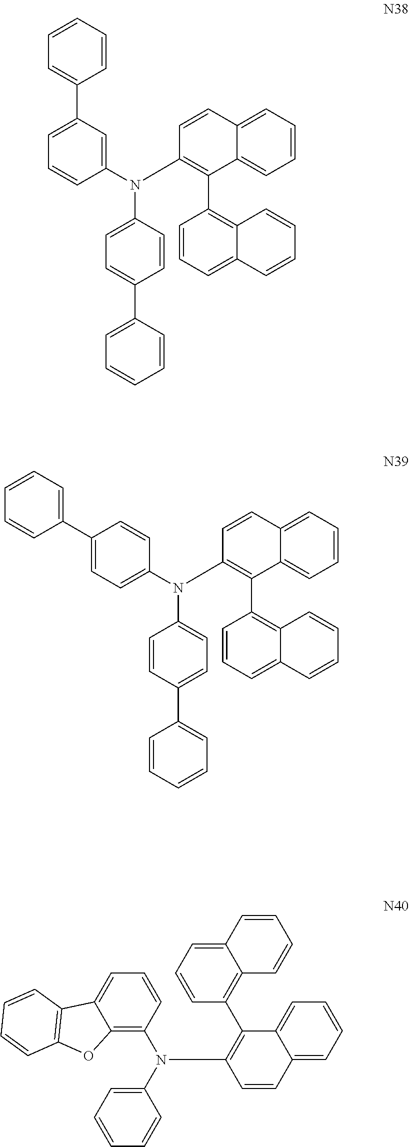 [SMILES: c1ccc(-c2ccc(N(c3cccc(-c4ccccc4)c3)c3ccc4ccccc4c3-c3cccc4ccccc34)cc2)cc1 ; c1ccc(-c2ccc(N(c3ccc(-c4ccccc4)cc3)c3ccc4ccccc4c3-c3cccc4ccccc34)cc2)cc1 ; c1ccc(N(c2ccc3ccccc3c2-c2cccc3ccccc23)c2cccc3c2oc2ccccc23)cc1]

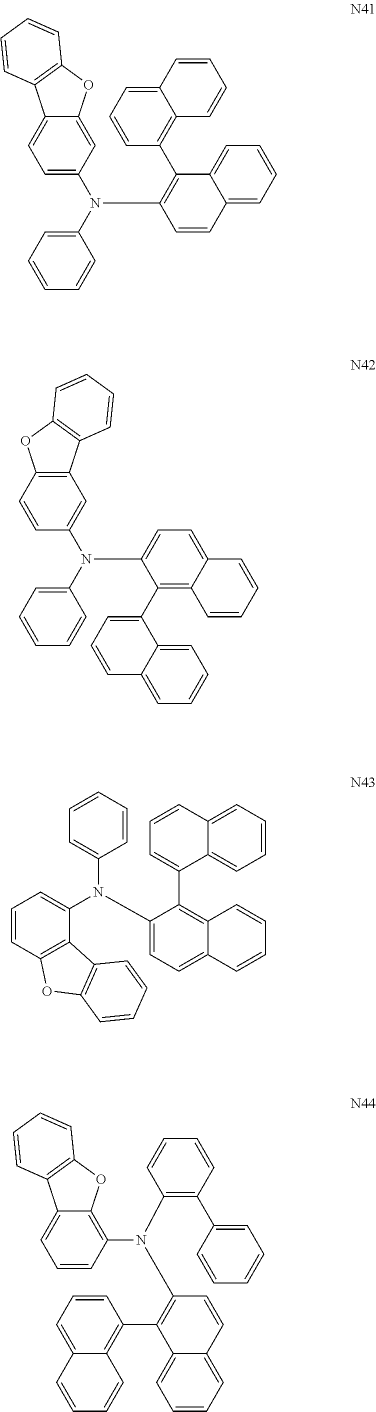 [SMILES: c1ccc(N(c2ccc3c(c2)oc2ccccc23)c2ccc3ccccc3c2-c2cccc3ccccc23)cc1 ; c1ccc(N(c2ccc3oc4ccccc4c3c2)c2ccc3ccccc3c2-c2cccc3ccccc23)cc1 ; c1ccc(N(c2ccc3ccccc3c2-c2cccc3ccccc23)c2cccc3oc4ccccc4c23)cc1 ; c1ccc(-c2ccccc2N(c2ccc3ccccc3c2-c2cccc3ccccc23)c2cccc3c2oc2ccccc23)cc1]

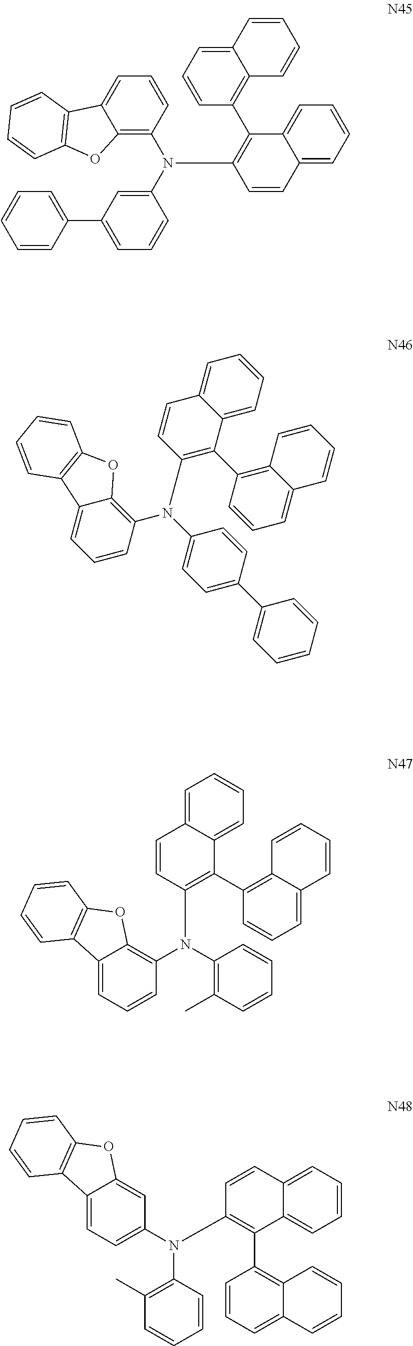 [SMILES: c1ccc(-c2cccc(N(c3ccc4ccccc4c3-c3cccc4ccccc34)c3cccc4c3oc3ccccc34)c2)cc1 ; c1ccc(-c2ccc(N(c3ccc4ccccc4c3-c3cccc4ccccc34)c3cccc4c3oc3ccccc34)cc2)cc1 ; Cc1ccccc1N(c1ccc2ccccc2c1-c1cccc2ccccc12)c1cccc2c1oc1ccccc12 ; Cc1ccccc1N(c1ccc2c(c1)oc1ccccc12)c1ccc2ccccc2c1-c1cccc2ccccc12]

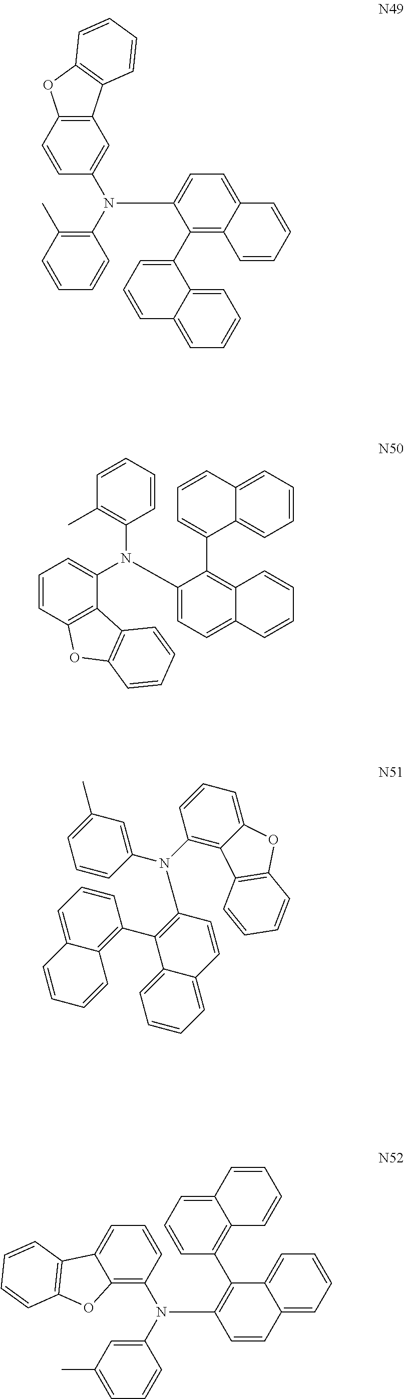 [SMILES: Cc1ccccc1N(c1ccc2oc3ccccc3c2c1)c1ccc2ccccc2c1-c1cccc2ccccc12 ; Cc1ccccc1N(c1ccc2ccccc2c1-c1cccc2ccccc12)c1cccc2oc3ccccc3c12 ; Cc1cccc(N(c2ccc3ccccc3c2-c2cccc3ccccc23)c2cccc3oc4ccccc4c23)c1 ; Cc1cccc(N(c2ccc3ccccc3c2-c2cccc3ccccc23)c2cccc3c2oc2ccccc23)c1]

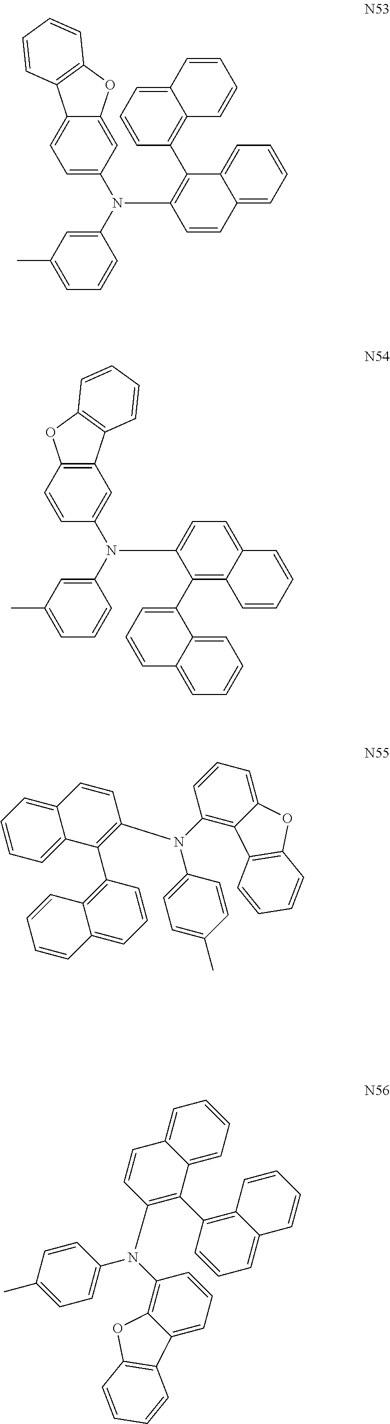 [SMILES: Cc1cccc(N(c2ccc3c(c2)oc2ccccc23)c2ccc3ccccc3c2-c2cccc3ccccc23)c1 ; Cc1cccc(N(c2ccc3oc4ccccc4c3c2)c2ccc3ccccc3c2-c2cccc3ccccc23)c1 ; Cc1ccc(N(c2ccc3ccccc3c2-c2cccc3ccccc23)c2cccc3oc4ccccc4c23)cc1 ; Cc1ccc(N(c2ccc3ccccc3c2-c2cccc3ccccc23)c2cccc3c2oc2ccccc23)cc1]

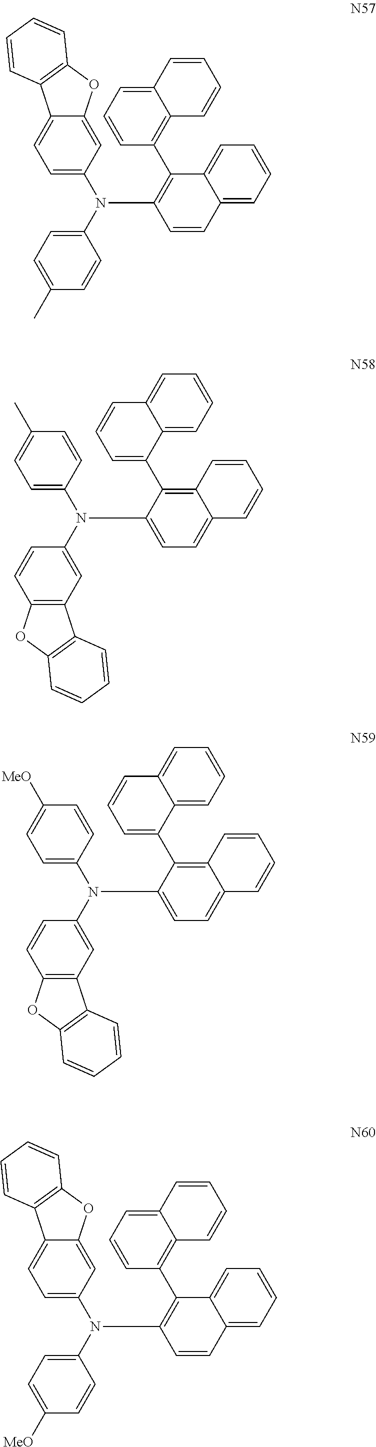 [SMILES: Cc1ccc(N(c2ccc3c(c2)oc2ccccc23)c2ccc3ccccc3c2-c2cccc3ccccc23)cc1 ; Cc1ccc(N(c2ccc3oc4ccccc4c3c2)c2ccc3ccccc3c2-c2cccc3ccccc23)cc1 ; COc1ccc(N(c2ccc3oc4ccccc4c3c2)c2ccc3ccccc3c2-c2cccc3ccccc23)cc1 ; COc1ccc(N(c2ccc3c(c2)oc2ccccc23)c2ccc3ccccc3c2-c2cccc3ccccc23)cc1]

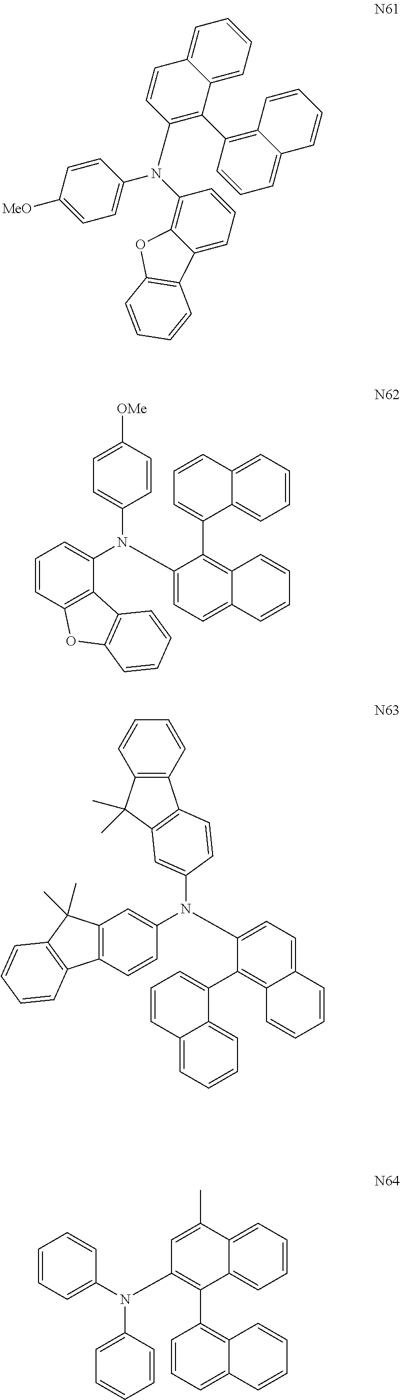 [SMILES: COc1ccc(N(c2ccc3ccccc3c2-c2cccc3ccccc23)c2cccc3c2oc2ccccc23)cc1 ; COc1ccc(N(c2ccc3ccccc3c2-c2cccc3ccccc23)c2cccc3oc4ccccc4c23)cc1 ; CC1(C)c2ccccc2-c2ccc(N(c3ccc4c(c3)C(C)(C)c3ccccc3-4)c3ccc4ccccc4c3-c3cccc4ccccc34)cc21 ; Cc1cc(N(c2ccccc2)c2ccccc2)c(-c2cccc3ccccc23)c2ccccc12]

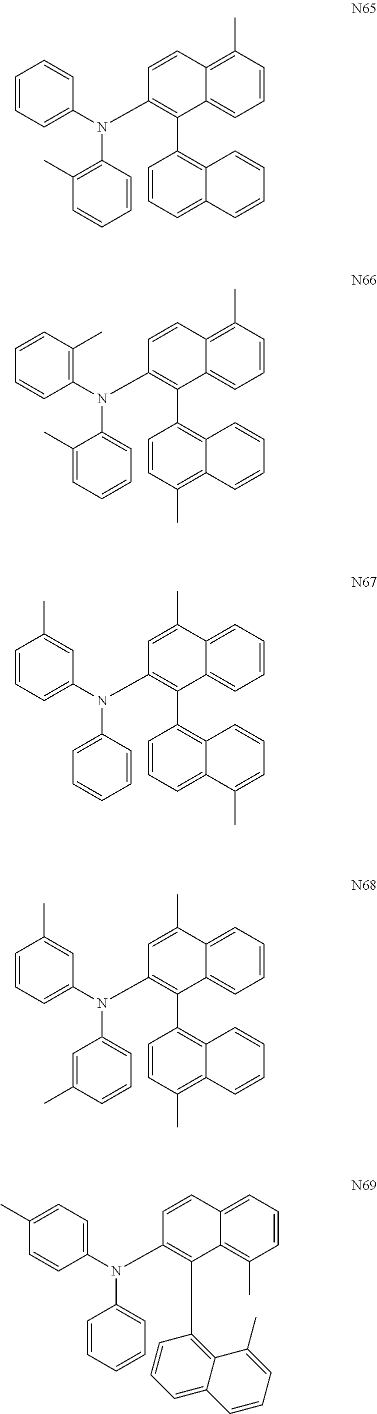 [SMILES: Cc1ccccc1N(c1ccccc1)c1ccc2c(C)cccc2c1-c1cccc2ccccc12 ; Cc1ccccc1N(c1ccccc1C)c1ccc2c(C)cccc2c1-c1ccc(C)c2ccccc12 ; Cc1cccc(N(c2ccccc2)c2cc(C)c3ccccc3c2-c2cccc3c(C)cccc23)c1 ; Cc1cccc(N(c2cccc(C)c2)c2cc(C)c3ccccc3c2-c2ccc(C)c3ccccc23)c1 ; Cc1ccc(N(c2ccccc2)c2ccc3cccc(C)c3c2-c2cccc3cccc(C)c23)cc1]

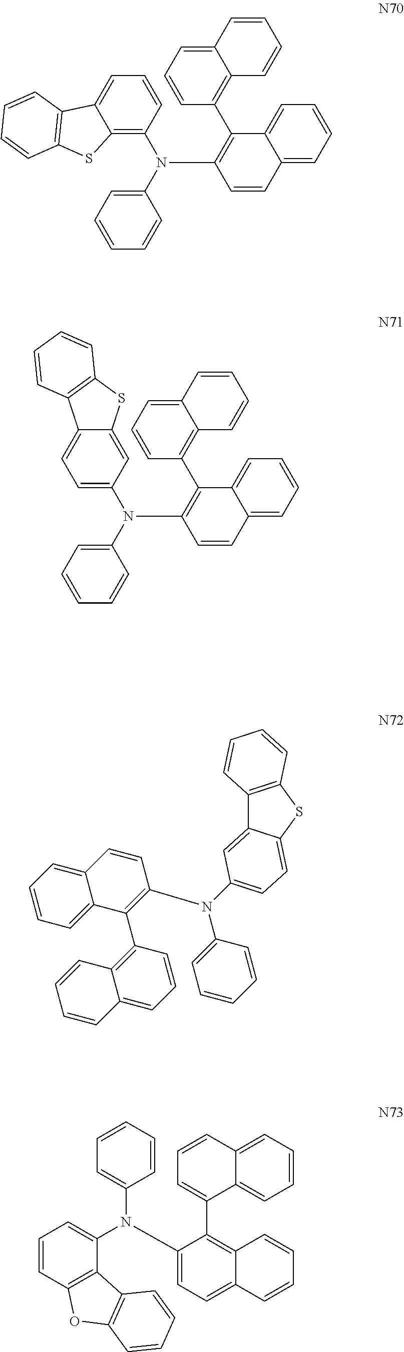 [SMILES: c1ccc(N(c2ccc3ccccc3c2-c2cccc3ccccc23)c2cccc3c2sc2ccccc23)cc1 ; c1ccc(N(c2ccc3c(c2)sc2ccccc23)c2ccc3ccccc3c2-c2cccc3ccccc23)cc1 ; c1ccc(N(c2ccc3sc4ccccc4c3c2)c2ccc3ccccc3c2-c2cccc3ccccc23)cc1 ; c1ccc(N(c2ccc3ccccc3c2-c2cccc3ccccc23)c2cccc3oc4ccccc4c23)cc1]

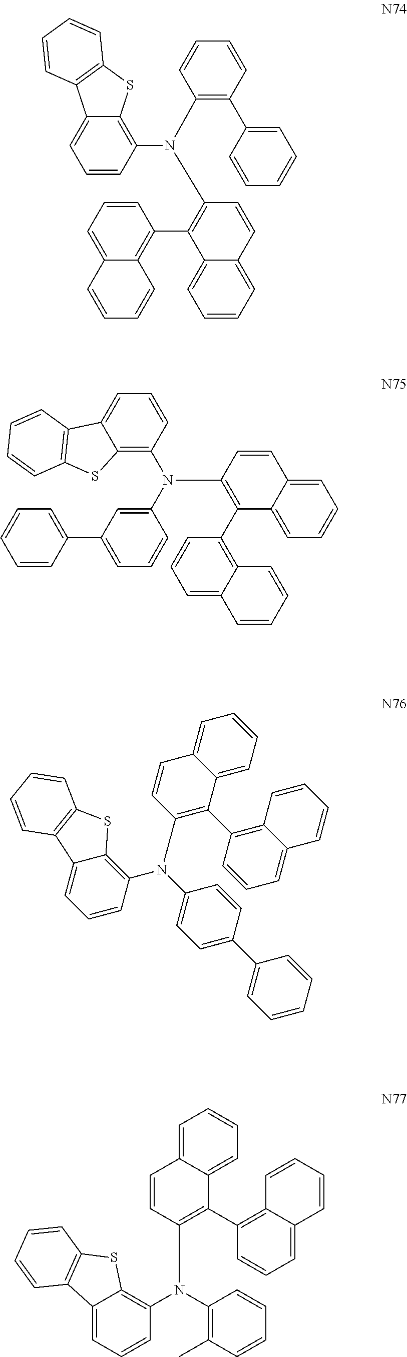 [SMILES: c1ccc(-c2ccccc2N(c2ccc3ccccc3c2-c2cccc3ccccc23)c2cccc3c2sc2ccccc23)cc1 ; c1ccc(-c2cccc(N(c3ccc4ccccc4c3-c3cccc4ccccc34)c3cccc4c3sc3ccccc34)c2)cc1 ; c1ccc(-c2ccc(N(c3ccc4ccccc4c3-c3cccc4ccccc34)c3cccc4c3sc3ccccc34)cc2)cc1 ; Cc1ccccc1N(c1ccc2ccccc2c1-c1cccc2ccccc12)c1cccc2c1sc1ccccc12]

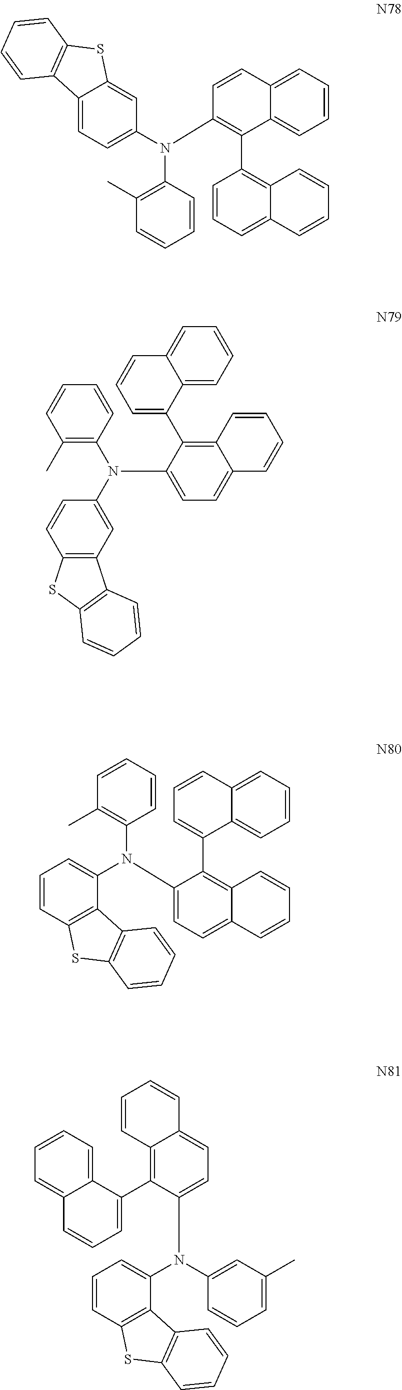 [SMILES: Cc1ccccc1N(c1ccc2c(c1)sc1ccccc12)c1ccc2ccccc2c1-c1cccc2ccccc12 ; Cc1ccccc1N(c1ccc2sc3ccccc3c2c1)c1ccc2ccccc2c1-c1cccc2ccccc12 ; Cc1ccccc1N(c1ccc2ccccc2c1-c1cccc2ccccc12)c1cccc2sc3ccccc3c12 ; Cc1cccc(N(c2ccc3ccccc3c2-c2cccc3ccccc23)c2cccc3sc4ccccc4c23)c1]

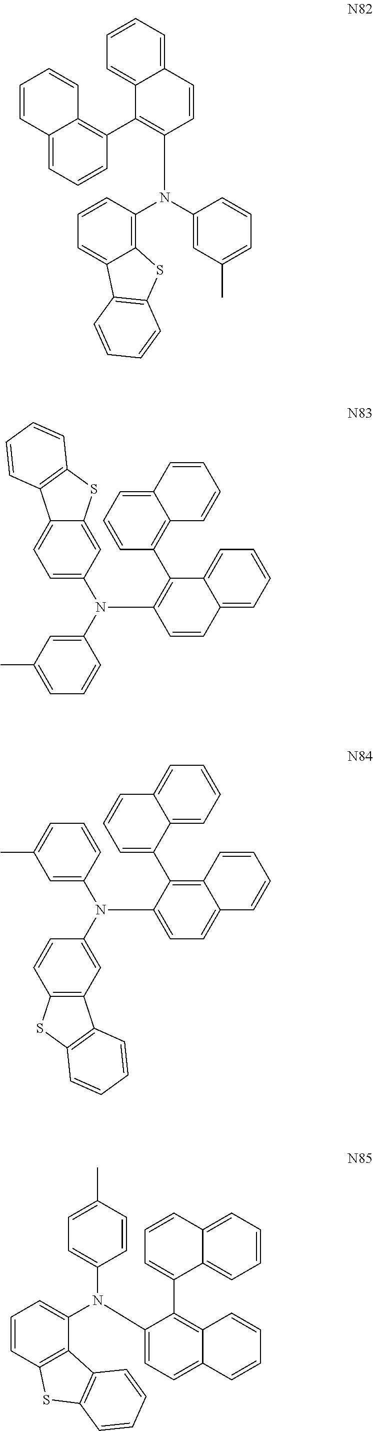 [SMILES: Cc1cccc(N(c2ccc3ccccc3c2-c2cccc3ccccc23)c2cccc3c2sc2ccccc23)c1 ; Cc1cccc(N(c2ccc3c(c2)sc2ccccc23)c2ccc3ccccc3c2-c2cccc3ccccc23)c1 ; Cc1cccc(N(c2ccc3sc4ccccc4c3c2)c2ccc3ccccc3c2-c2cccc3ccccc23)c1 ; Cc1ccc(N(c2ccc3ccccc3c2-c2cccc3ccccc23)c2cccc3sc4ccccc4c23)cc1]

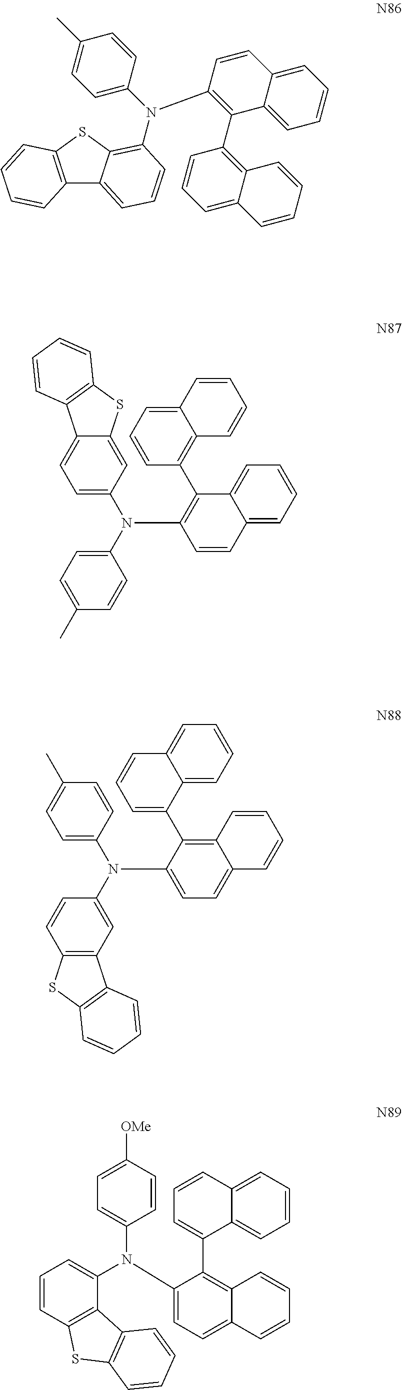 [SMILES: Cc1ccc(N(c2ccc3ccccc3c2-c2cccc3ccccc23)c2cccc3c2sc2ccccc23)cc1 ; Cc1ccc(N(c2ccc3c(c2)sc2ccccc23)c2ccc3ccccc3c2-c2cccc3ccccc23)cc1 ; Cc1ccc(N(c2ccc3sc4ccccc4c3c2)c2ccc3ccccc3c2-c2cccc3ccccc23)cc1 ; COc1ccc(N(c2ccc3ccccc3c2-c2cccc3ccccc23)c2cccc3sc4ccccc4c23)cc1]

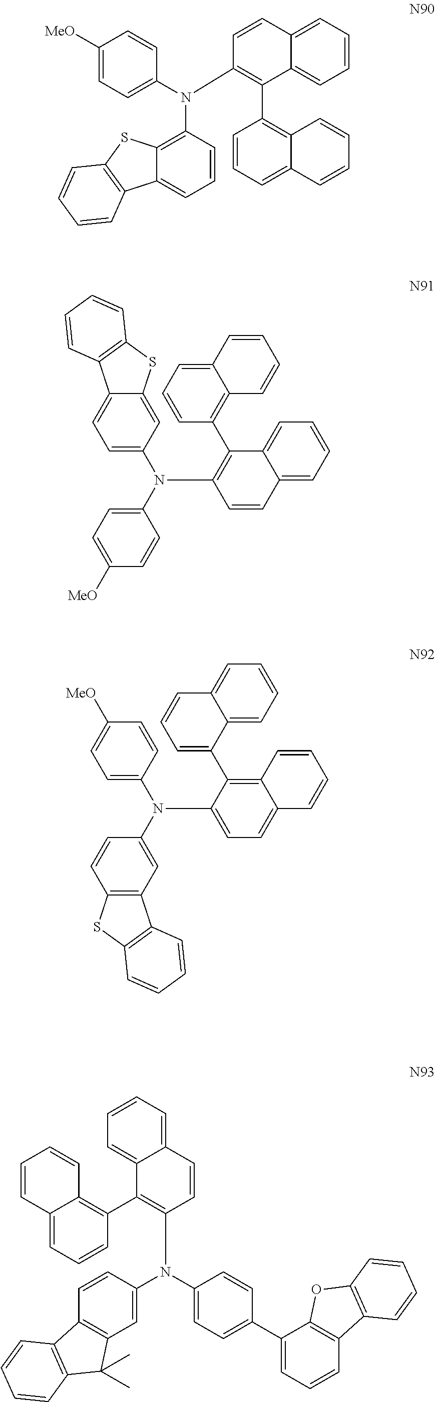 [SMILES: COc1ccc(N(c2ccc3ccccc3c2-c2cccc3ccccc23)c2cccc3c2sc2ccccc23)cc1 ; COc1ccc(N(c2ccc3c(c2)sc2ccccc23)c2ccc3ccccc3c2-c2cccc3ccccc23)cc1 ; COc1ccc(N(c2ccc3sc4ccccc4c3c2)c2ccc3ccccc3c2-c2cccc3ccccc23)cc1 ; CC1(C)c2ccccc2-c2ccc(N(c3ccc(-c4cccc5c4oc4ccccc45)cc3)c3ccc4ccccc4c3-c3cccc4ccccc34)cc21]

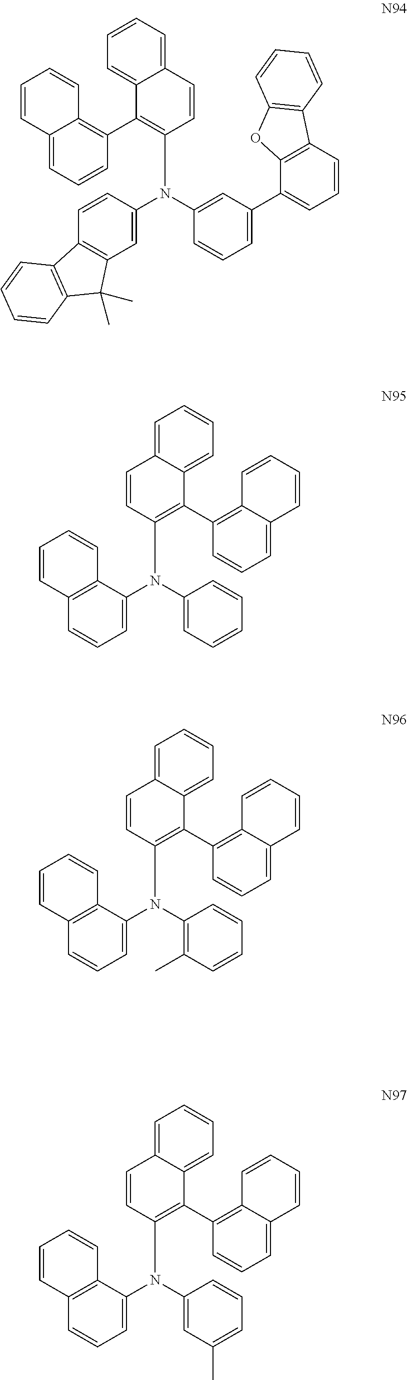 [SMILES: CC1(C)c2ccccc2-c2ccc(N(c3cccc(-c4cccc5c4oc4ccccc45)c3)c3ccc4ccccc4c3-c3cccc4ccccc34)cc21 ; c1ccc(N(c2ccc3ccccc3c2-c2cccc3ccccc23)c2cccc3ccccc23)cc1 ; Cc1ccccc1N(c1ccc2ccccc2c1-c1cccc2ccccc12)c1cccc2ccccc12 ; Cc1cccc(N(c2ccc3ccccc3c2-c2cccc3ccccc23)c2cccc3ccccc23)c1]

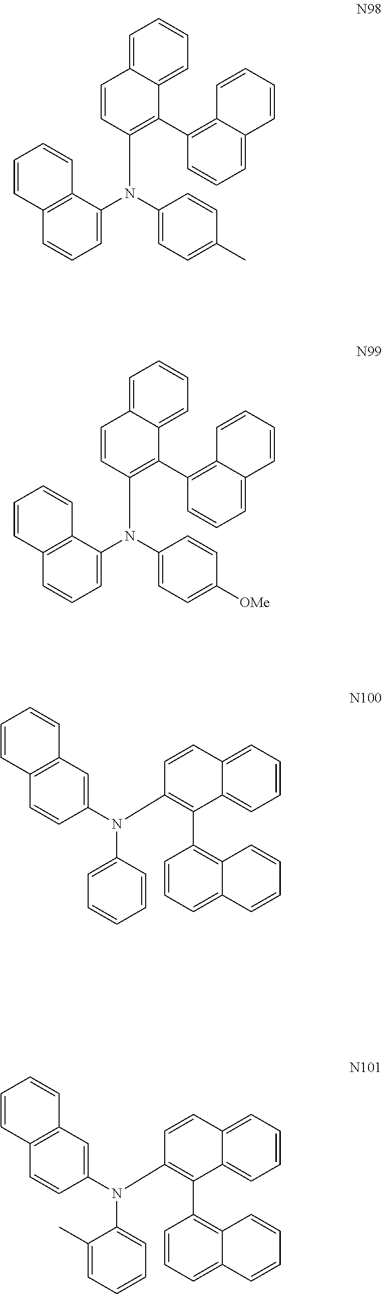 [SMILES: Cc1ccc(N(c2ccc3ccccc3c2-c2cccc3ccccc23)c2cccc3ccccc23)cc1 ; COc1ccc(N(c2ccc3ccccc3c2-c2cccc3ccccc23)c2cccc3ccccc23)cc1 ; c1ccc(N(c2ccc3ccccc3c2)c2ccc3ccccc3c2-c2cccc3ccccc23)cc1 ; Cc1ccccc1N(c1ccc2ccccc2c1)c1ccc2ccccc2c1-c1cccc2ccccc12]

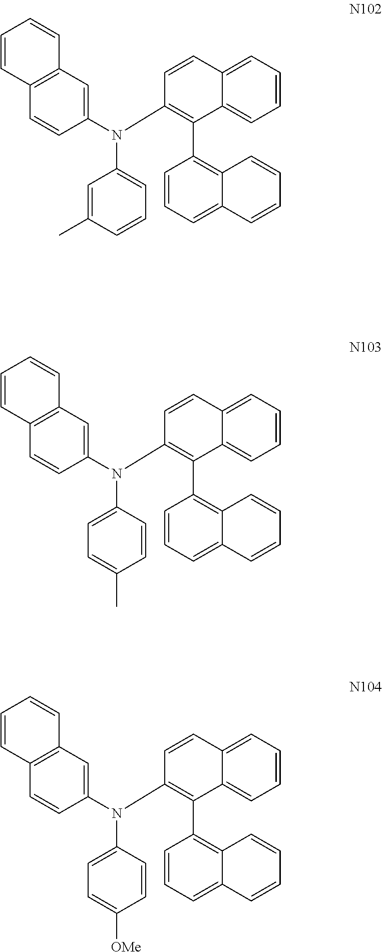 [SMILES: Cc1cccc(N(c2ccc3ccccc3c2)c2ccc3ccccc3c2-c2cccc3ccccc23)c1 ; Cc1ccc(N(c2ccc3ccccc3c2)c2ccc3ccccc3c2-c2cccc3ccccc23)cc1 ; COc1ccc(N(c2ccc3ccccc3c2)c2ccc3ccccc3c2-c2cccc3ccccc23)cc1]

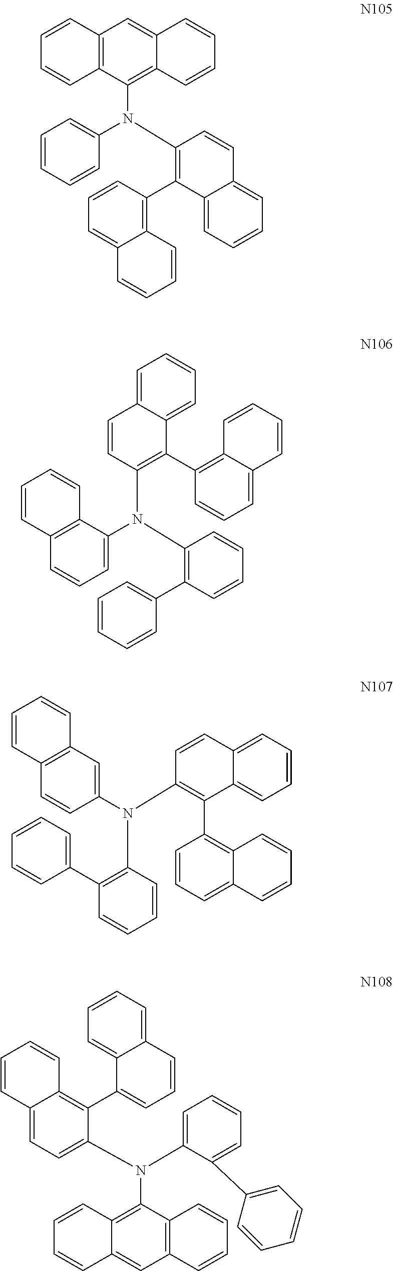 [SMILES: c1ccc(N(c2ccc3ccccc3c2-c2cccc3ccccc23)c2c3ccccc3cc3ccccc23)cc1 ; c1ccc(-c2ccccc2N(c2ccc3ccccc3c2-c2cccc3ccccc23)c2cccc3ccccc23)cc1 ; c1ccc(-c2ccccc2N(c2ccc3ccccc3c2)c2ccc3ccccc3c2-c2cccc3ccccc23)cc1 ; c1ccc(-c2ccccc2N(c2ccc3ccccc3c2-c2cccc3ccccc23)c2c3ccccc3cc3ccccc23)cc1]

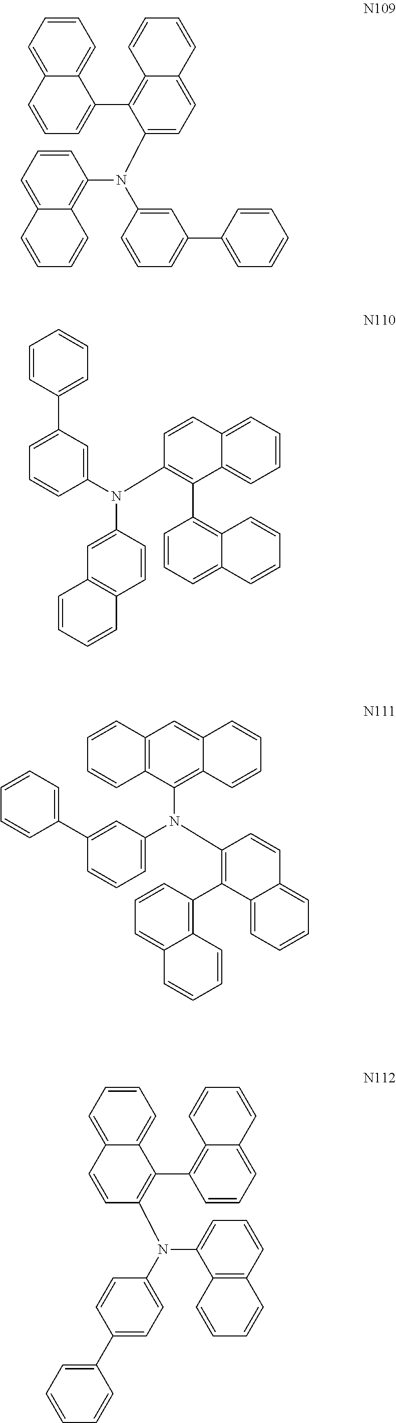 [SMILES: c1ccc(-c2cccc(N(c3ccc4ccccc4c3-c3cccc4ccccc34)c3cccc4ccccc34)c2)cc1 ; c1ccc(-c2cccc(N(c3ccc4ccccc4c3)c3ccc4ccccc4c3-c3cccc4ccccc34)c2)cc1 ; c1ccc(-c2cccc(N(c3ccc4ccccc4c3-c3cccc4ccccc34)c3c4ccccc4cc4ccccc34)c2)cc1 ; c1ccc(-c2ccc(N(c3ccc4ccccc4c3-c3cccc4ccccc34)c3cccc4ccccc34)cc2)cc1]

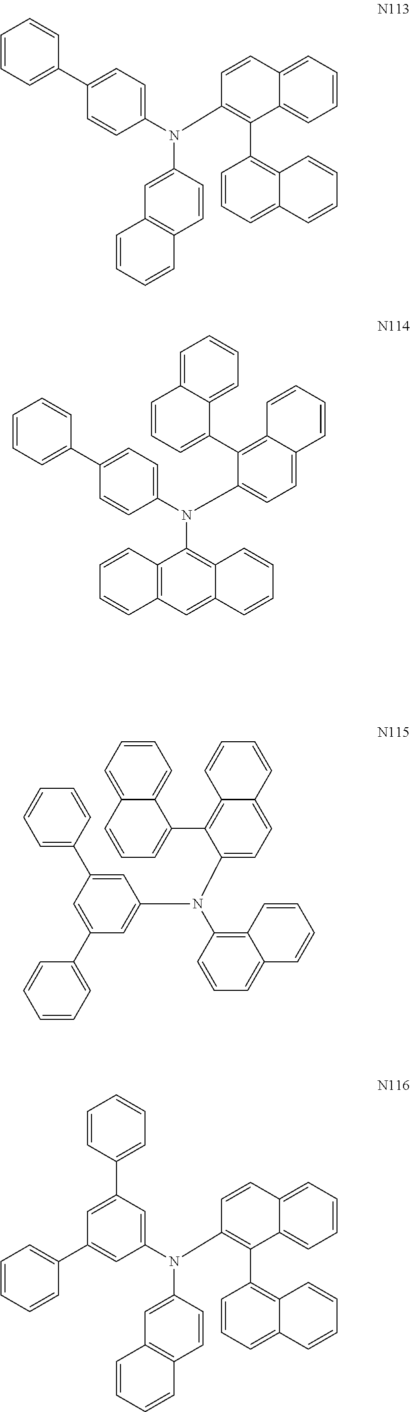 [SMILES: c1ccc(-c2ccc(N(c3ccc4ccccc4c3)c3ccc4ccccc4c3-c3cccc4ccccc34)cc2)cc1 ; c1ccc(-c2ccc(N(c3ccc4ccccc4c3-c3cccc4ccccc34)c3c4ccccc4cc4ccccc34)cc2)cc1 ; c1ccc(-c2cc(-c3ccccc3)cc(N(c3ccc4ccccc4c3-c3cccc4ccccc34)c3cccc4ccccc34)c2)cc1 ; c1ccc(-c2cc(-c3ccccc3)cc(N(c3ccc4ccccc4c3)c3ccc4ccccc4c3-c3cccc4ccccc34)c2)cc1]

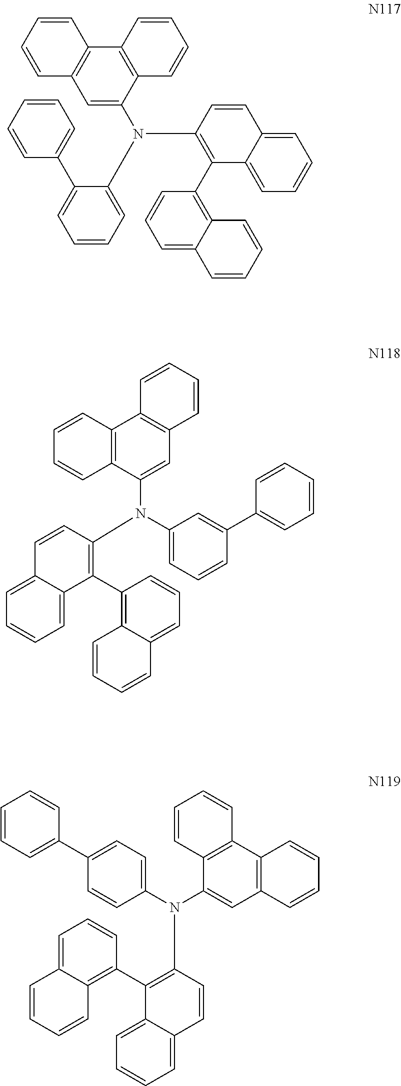 [SMILES: c1ccc(-c2ccccc2N(c2ccc3ccccc3c2-c2cccc3ccccc23)c2cc3ccccc3c3ccccc23)cc1 ; c1ccc(-c2cccc(N(c3ccc4ccccc4c3-c3cccc4ccccc34)c3cc4ccccc4c4ccccc34)c2)cc1 ; c1ccc(-c2ccc(N(c3ccc4ccccc4c3-c3cccc4ccccc34)c3cc4ccccc4c4ccccc34)cc2)cc1]

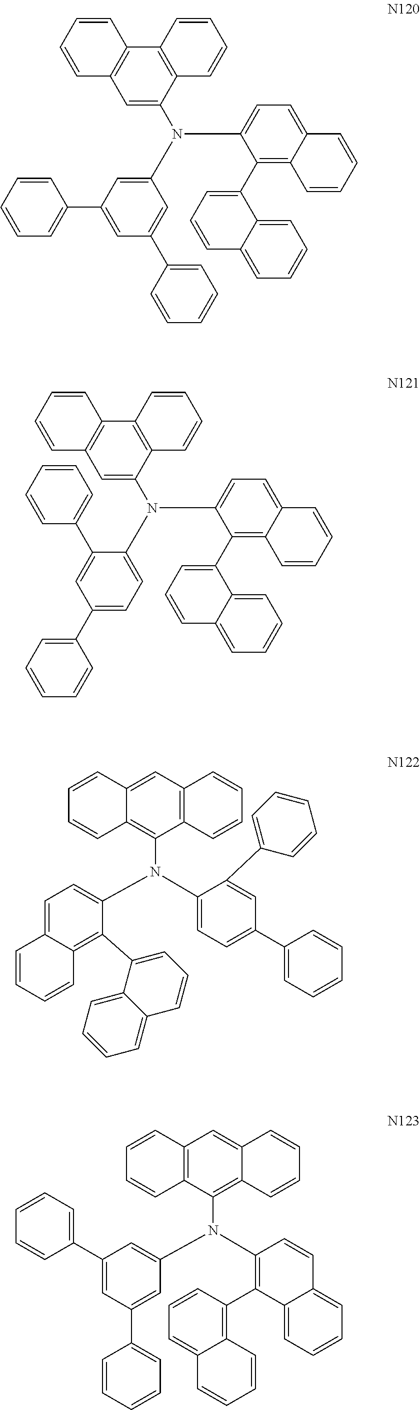 [SMILES: c1ccc(-c2cc(-c3ccccc3)cc(N(c3ccc4ccccc4c3-c3cccc4ccccc34)c3cc4ccccc4c4ccccc34)c2)cc1 ; c1ccc(-c2ccc(N(c3ccc4ccccc4c3-c3cccc4ccccc34)c3cc4ccccc4c4ccccc34)c(-c3ccccc3)c2)cc1 ; c1ccc(-c2ccc(N(c3ccc4ccccc4c3-c3cccc4ccccc34)c3c4ccccc4cc4ccccc34)c(-c3ccccc3)c2)cc1 ; c1ccc(-c2cc(-c3ccccc3)cc(N(c3ccc4ccccc4c3-c3cccc4ccccc34)c3c4ccccc4cc4ccccc34)c2)cc1]

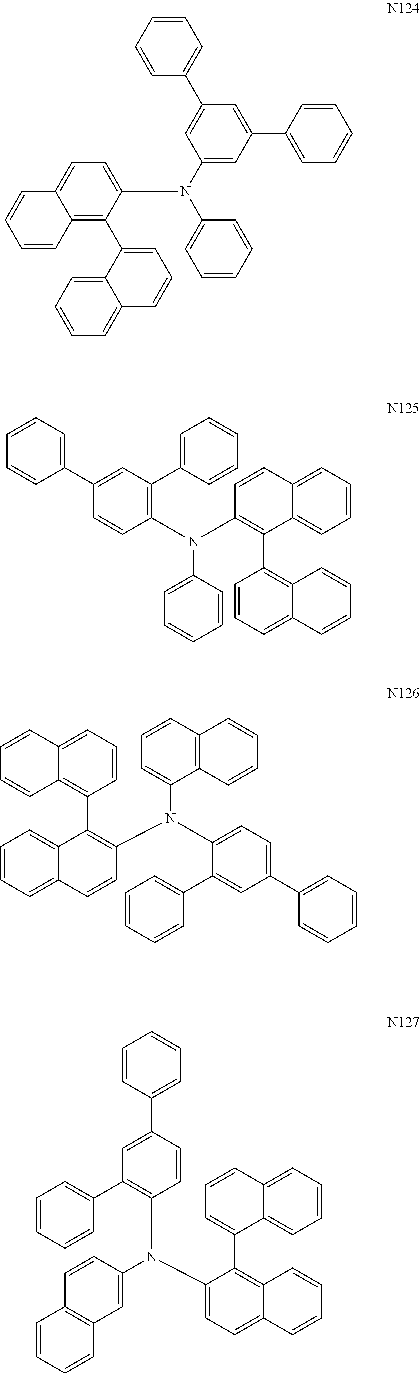 [SMILES: c1ccc(-c2cc(-c3ccccc3)cc(N(c3ccccc3)c3ccc4ccccc4c3-c3cccc4ccccc34)c2)cc1 ; c1ccc(-c2ccc(N(c3ccccc3)c3ccc4ccccc4c3-c3cccc4ccccc34)c(-c3ccccc3)c2)cc1 ; c1ccc(-c2ccc(N(c3ccc4ccccc4c3-c3cccc4ccccc34)c3cccc4ccccc34)c(-c3ccccc3)c2)cc1 ; c1ccc(-c2ccc(N(c3ccc4ccccc4c3)c3ccc4ccccc4c3-c3cccc4ccccc34)c(-c3ccccc3)c2)cc1]

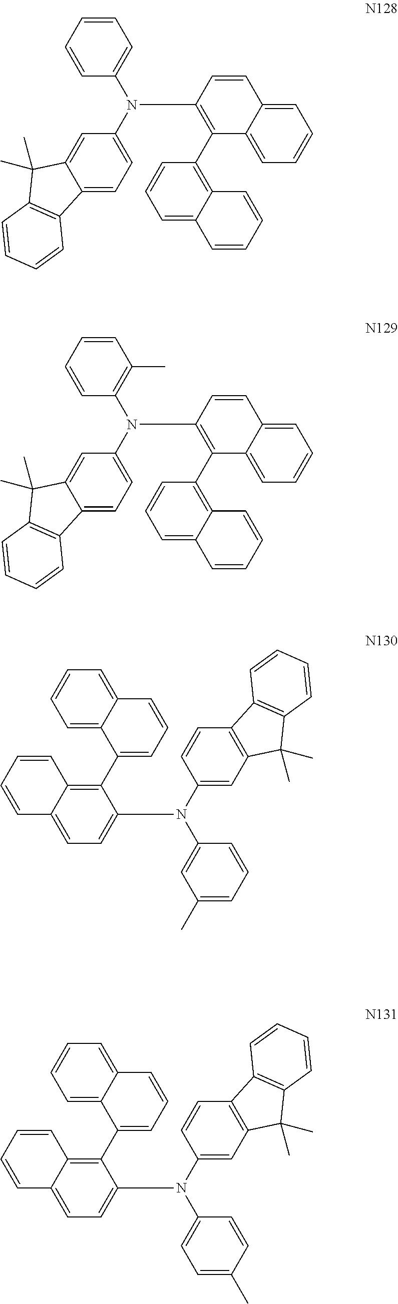 [SMILES: CC1(C)c2ccccc2-c2ccc(N(c3ccccc3)c3ccc4ccccc4c3-c3cccc4ccccc34)cc21 ; Cc1ccccc1N(c1ccc2c(c1)C(C)(C)c1ccccc1-2)c1ccc2ccccc2c1-c1cccc2ccccc12 ; Cc1cccc(N(c2ccc3c(c2)C(C)(C)c2ccccc2-3)c2ccc3ccccc3c2-c2cccc3ccccc23)c1 ; Cc1ccc(N(c2ccc3c(c2)C(C)(C)c2ccccc2-3)c2ccc3ccccc3c2-c2cccc3ccccc23)cc1]

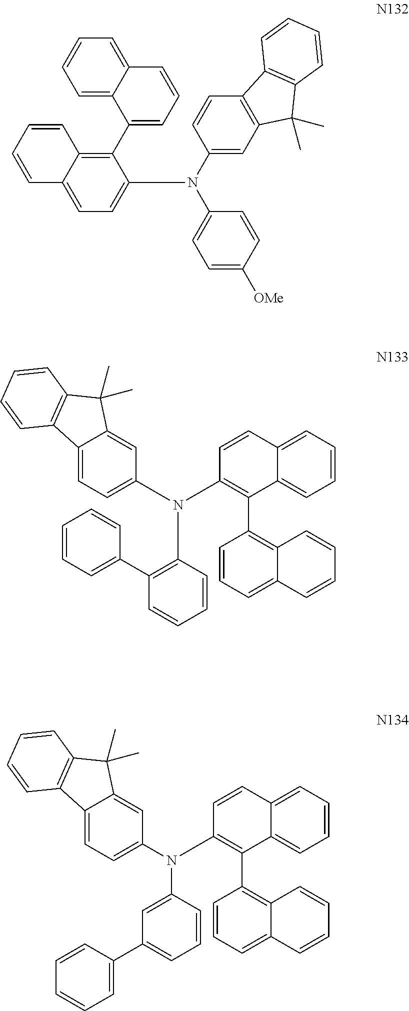 [SMILES: COc1ccc(N(c2ccc3c(c2)C(C)(C)c2ccccc2-3)c2ccc3ccccc3c2-c2cccc3ccccc23)cc1 ; CC1(C)c2ccccc2-c2ccc(N(c3ccccc3-c3ccccc3)c3ccc4ccccc4c3-c3cccc4ccccc34)cc21 ; CC1(C)c2ccccc2-c2ccc(N(c3cccc(-c4ccccc4)c3)c3ccc4ccccc4c3-c3cccc4ccccc34)cc21]

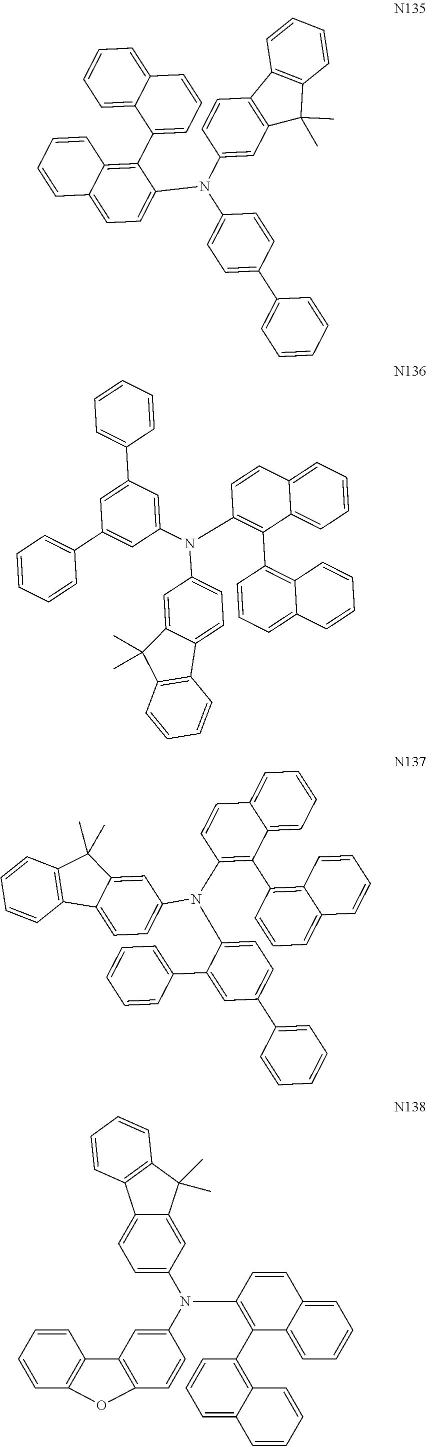 [SMILES: CC1(C)c2ccccc2-c2ccc(N(c3ccc(-c4ccccc4)cc3)c3ccc4ccccc4c3-c3cccc4ccccc34)cc21 ; CC1(C)c2ccccc2-c2ccc(N(c3cc(-c4ccccc4)cc(-c4ccccc4)c3)c3ccc4ccccc4c3-c3cccc4ccccc34)cc21 ; CC1(C)c2ccccc2-c2ccc(N(c3ccc(-c4ccccc4)cc3-c3ccccc3)c3ccc4ccccc4c3-c3cccc4ccccc34)cc21 ; CC1(C)c2ccccc2-c2ccc(N(c3ccc4oc5ccccc5c4c3)c3ccc4ccccc4c3-c3cccc4ccccc34)cc21]

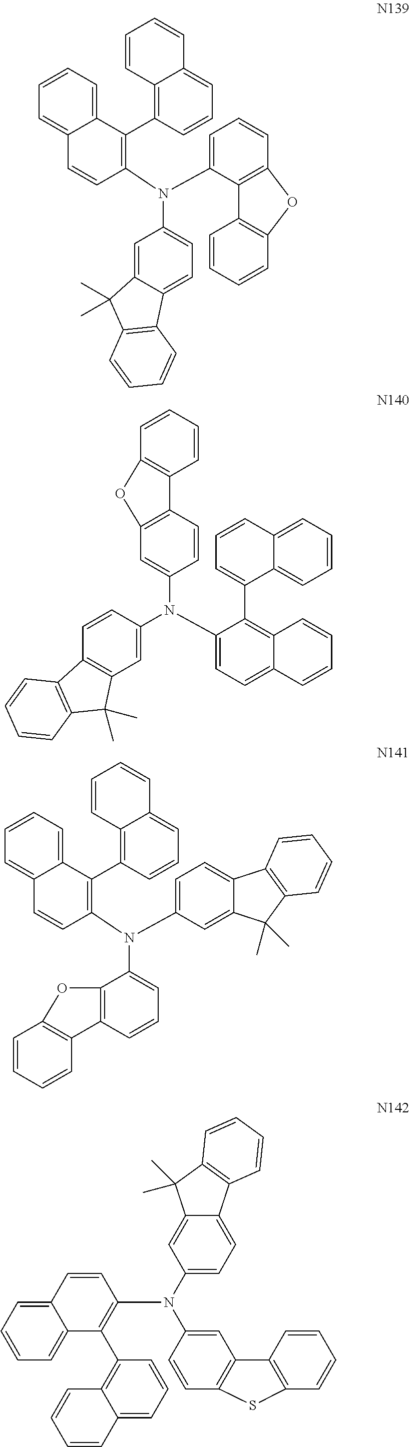

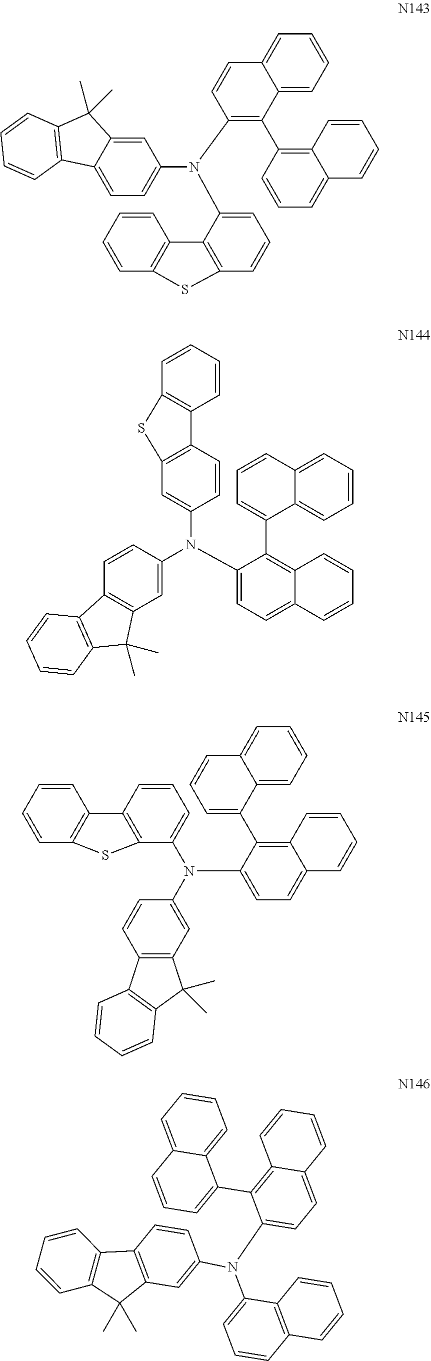 [SMILES: CC1(C)c2ccccc2-c2ccc(N(c3ccc4ccccc4c3-c3cccc4ccccc34)c3cccc4sc5ccccc5c34)cc21 ; CC1(C)c2ccccc2-c2ccc(N(c3ccc4c(c3)sc3ccccc34)c3ccc4ccccc4c3-c3cccc4ccccc34)cc21 ; CC1(C)c2ccccc2-c2ccc(N(c3ccc4ccccc4c3-c3cccc4ccccc34)c3cccc4c3sc3ccccc34)cc21 ; CC1(C)c2ccccc2-c2ccc(N(c3ccc4ccccc4c3-c3cccc4ccccc34)c3cccc4ccccc34)cc21]

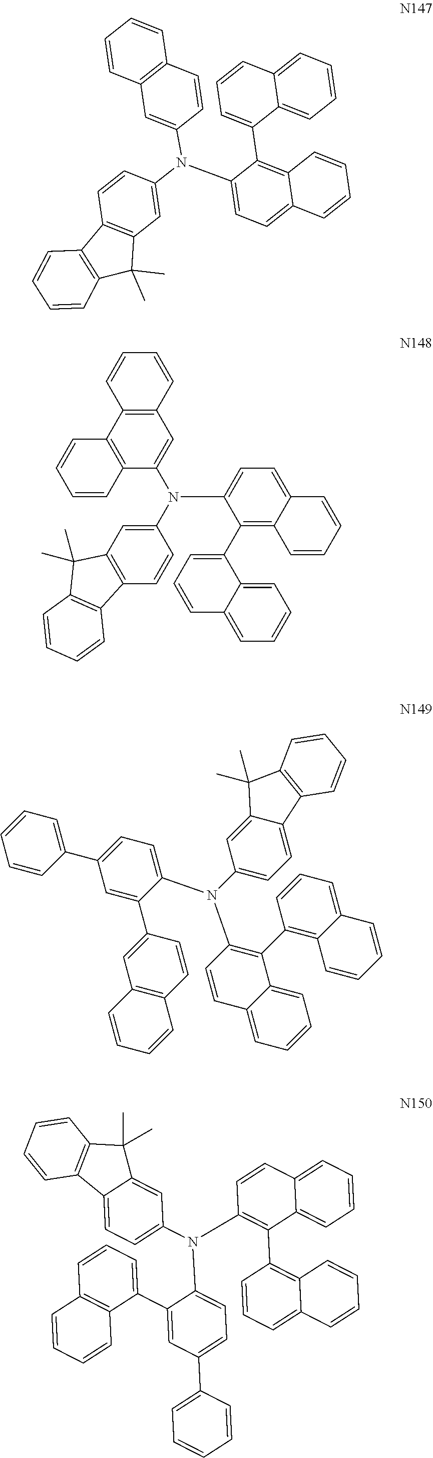 [SMILES: CC1(C)c2ccccc2-c2ccc(N(c3ccc4ccccc4c3)c3ccc4ccccc4c3-c3cccc4ccccc34)cc21 ; CC1(C)c2ccccc2-c2ccc(N(c3ccc4ccccc4c3-c3cccc4ccccc34)c3cc4ccccc4c4ccccc34)cc21 ; CC1(C)c2ccccc2-c2ccc(N(c3ccc(-c4ccccc4)cc3-c3ccc4ccccc4c3)c3ccc4ccccc4c3-c3cccc4ccccc34)cc21 ; CC1(C)c2ccccc2-c2ccc(N(c3ccc(-c4ccccc4)cc3-c3cccc4ccccc34)c3ccc4ccccc4c3-c3cccc4ccccc34)cc21]

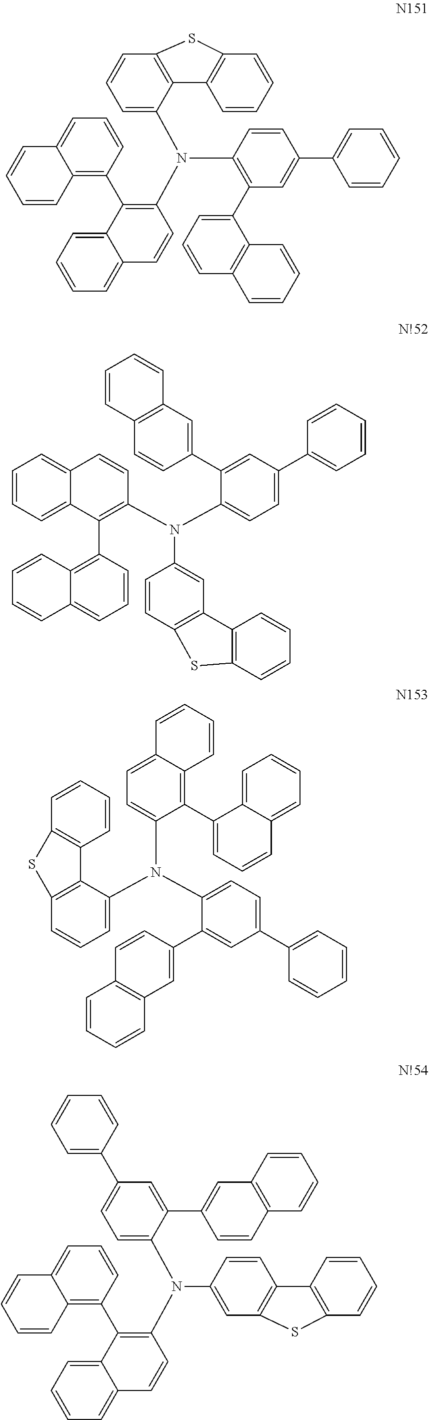

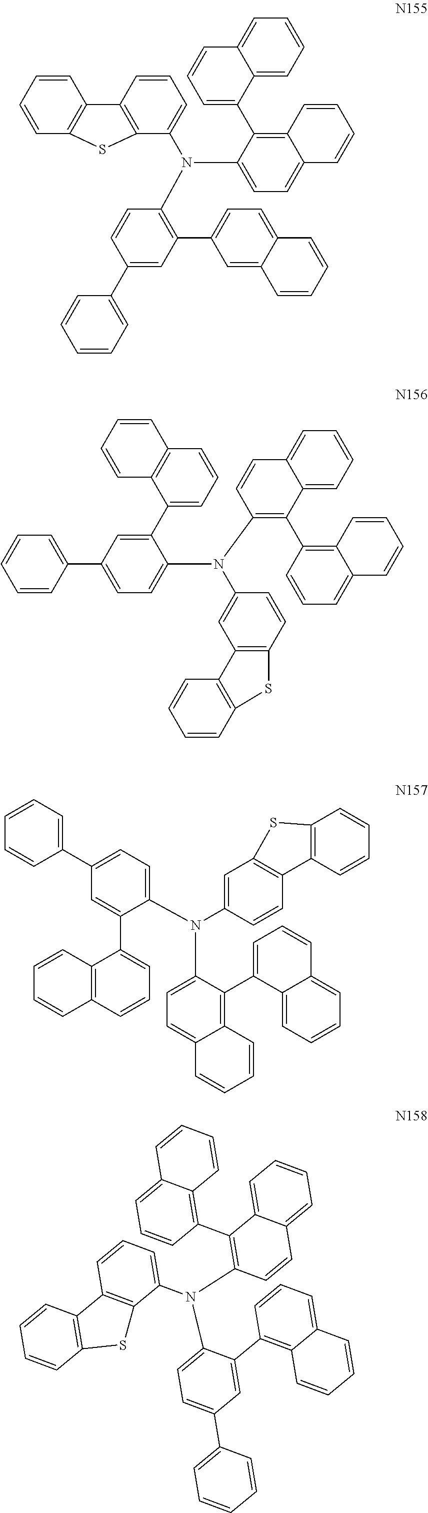

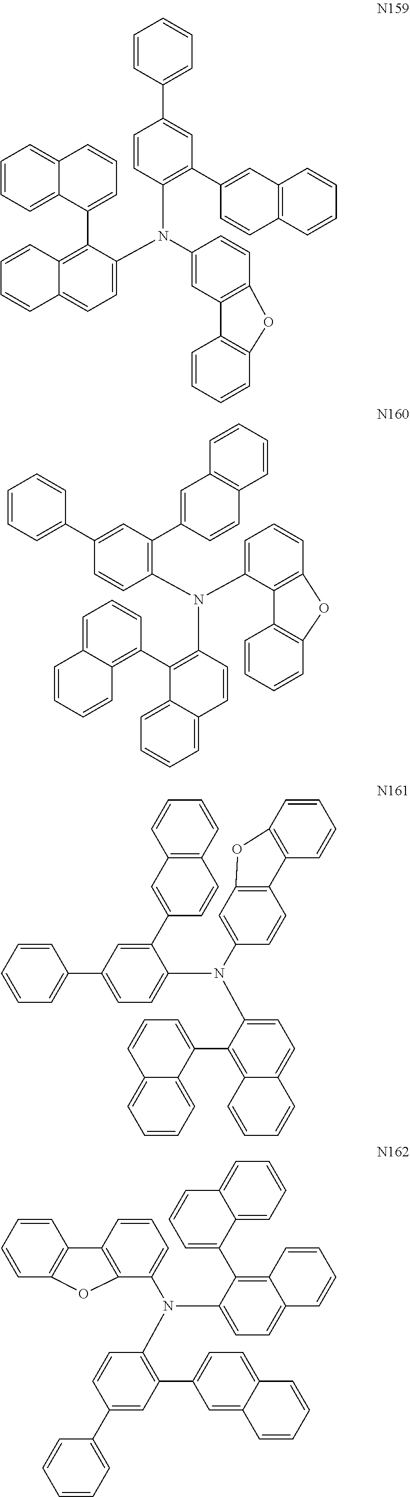

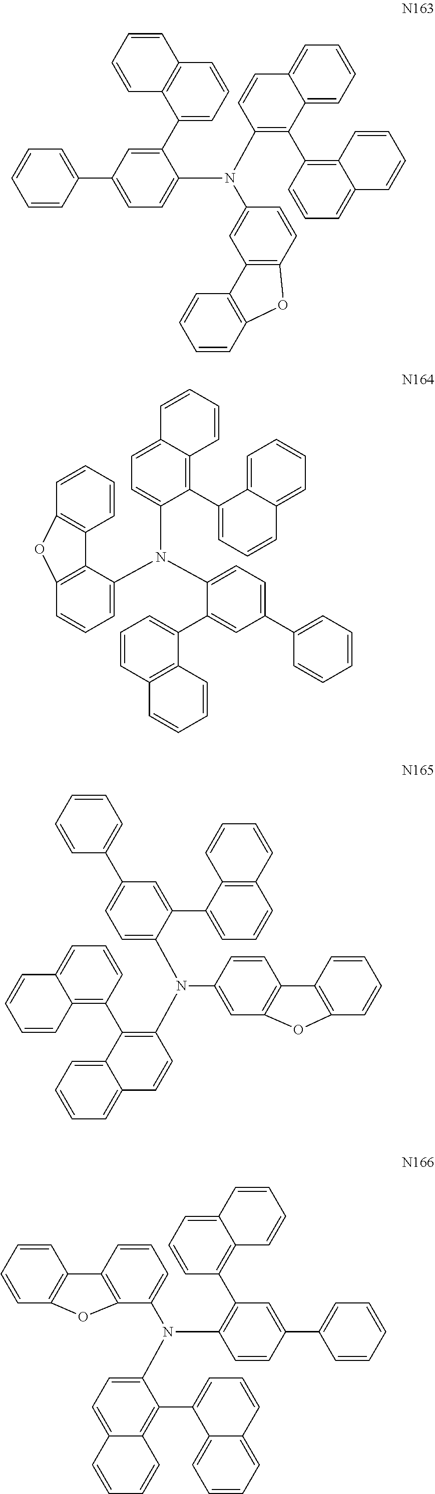

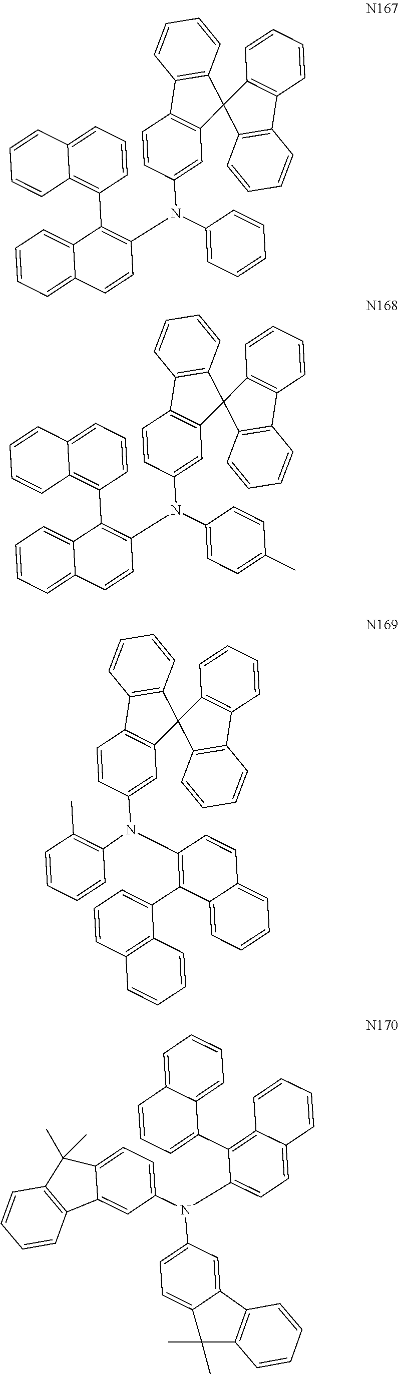

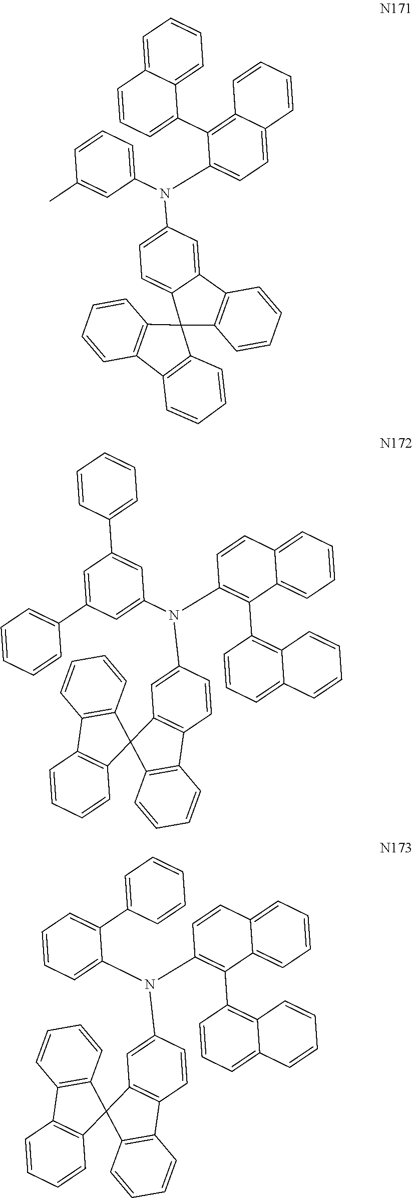

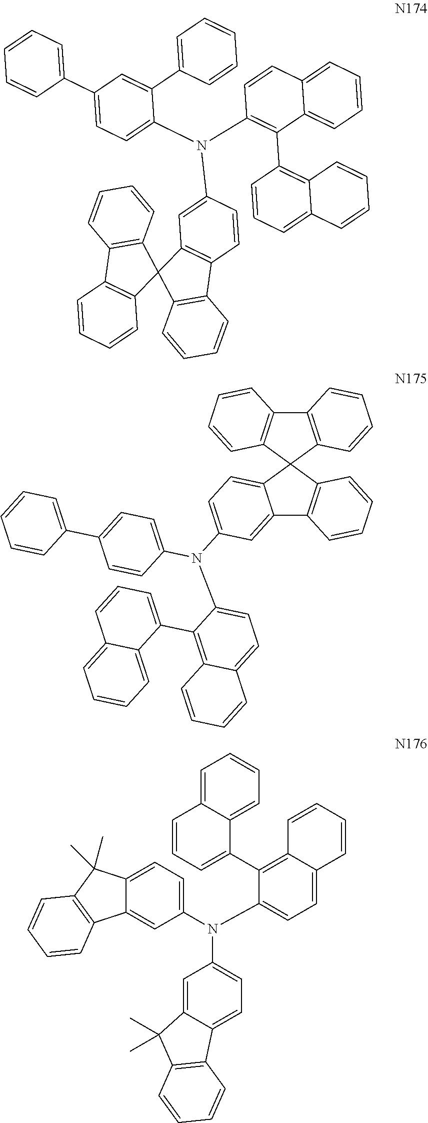

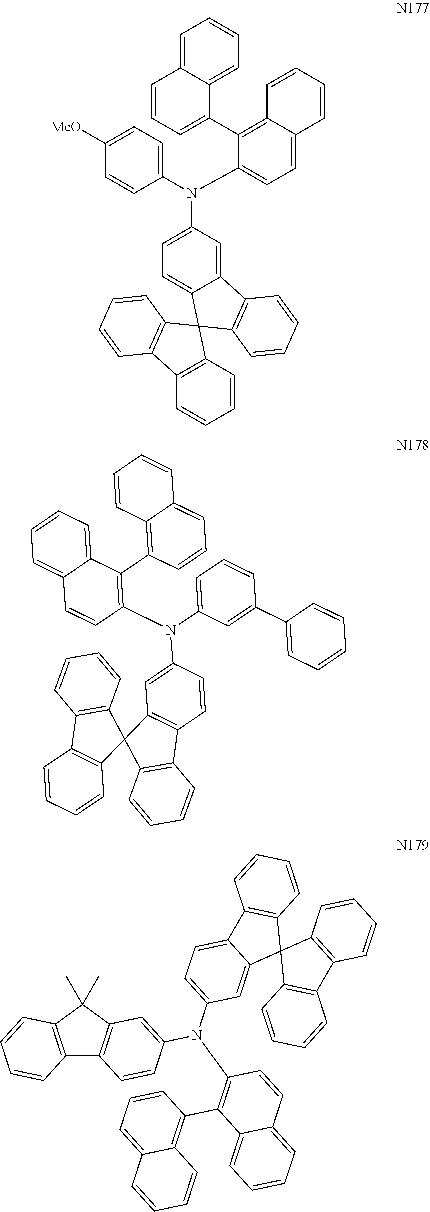 [SMILES: COc1ccc(N(c2ccc3c(c2)-c2ccccc2C32c3ccccc3-c3ccccc32)c2ccc3ccccc3c2-c2cccc3ccccc23)cc1 ; c1ccc(-c2cccc(N(c3ccc4c(c3)C3(c5ccccc5-c5ccccc53)c3ccccc3-4)c3ccc4ccccc4c3-c3cccc4ccccc34)c2)cc1 ; CC1(C)c2ccccc2-c2ccc(N(c3ccc4c(c3)C3(c5ccccc5-c5ccccc53)c3ccccc3-4)c3ccc4ccccc4c3-c3cccc4ccccc34)cc21]

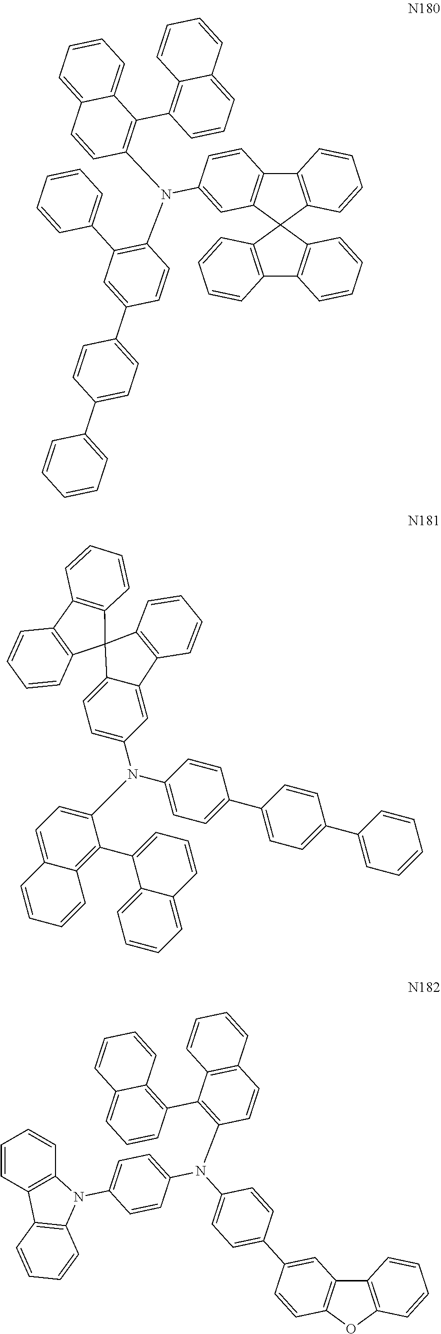 [SMILES: c1ccc(-c2ccc(-c3ccc(N(c4ccc5c(c4)C4(c6ccccc6-c6ccccc64)c4ccccc4-5)c4ccc5ccccc5c4-c4cccc5ccccc45)c(-c4ccccc4)c3)cc2)cc1 ; c1ccc(-c2ccc(-c3ccc(N(c4ccc5c(c4)-c4ccccc4C54c5ccccc5-c5ccccc54)c4ccc5ccccc5c4-c4cccc5ccccc45)cc3)cc2)cc1 ; c1ccc2c(-c3c(N(c4ccc(-c5ccc6oc7ccccc7c6c5)cc4)c4ccc(-n5c6ccccc6c6ccccc65)cc4)ccc4ccccc34)cccc2c1]

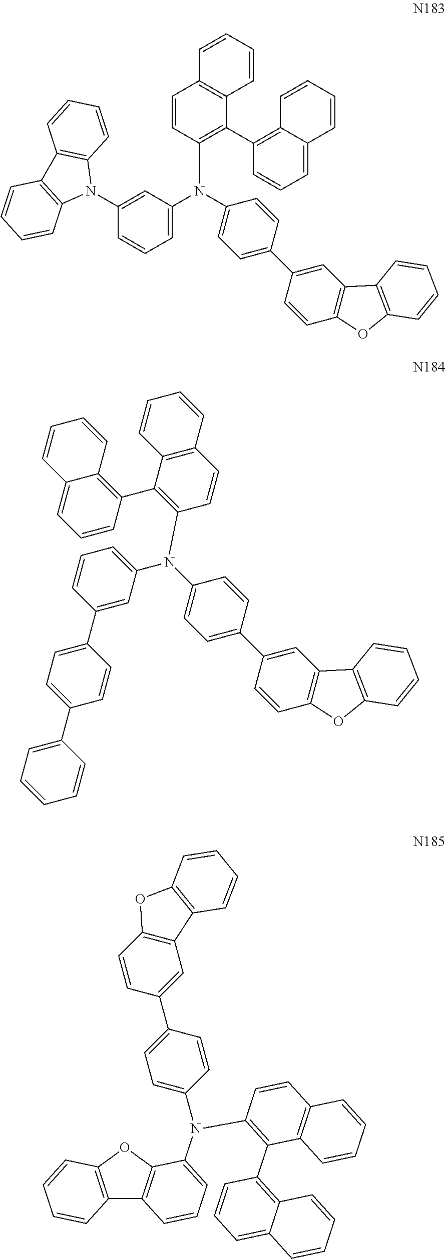 [SMILES: c1cc(N(c2ccc(-c3ccc4oc5ccccc5c4c3)cc2)c2ccc3ccccc3c2-c2cccc3ccccc23)cc(-n2c3ccccc3c3ccccc32)c1 ; c1ccc(-c2ccc(-c3cccc(N(c4ccc(-c5ccc6oc7ccccc7c6c5)cc4)c4ccc5ccccc5c4-c4cccc5ccccc45)c3)cc2)cc1 ; c1ccc2c(-c3c(N(c4ccc(-c5ccc6oc7ccccc7c6c5)cc4)c4cccc5c4oc4ccccc45)ccc4ccccc34)cccc2c1]

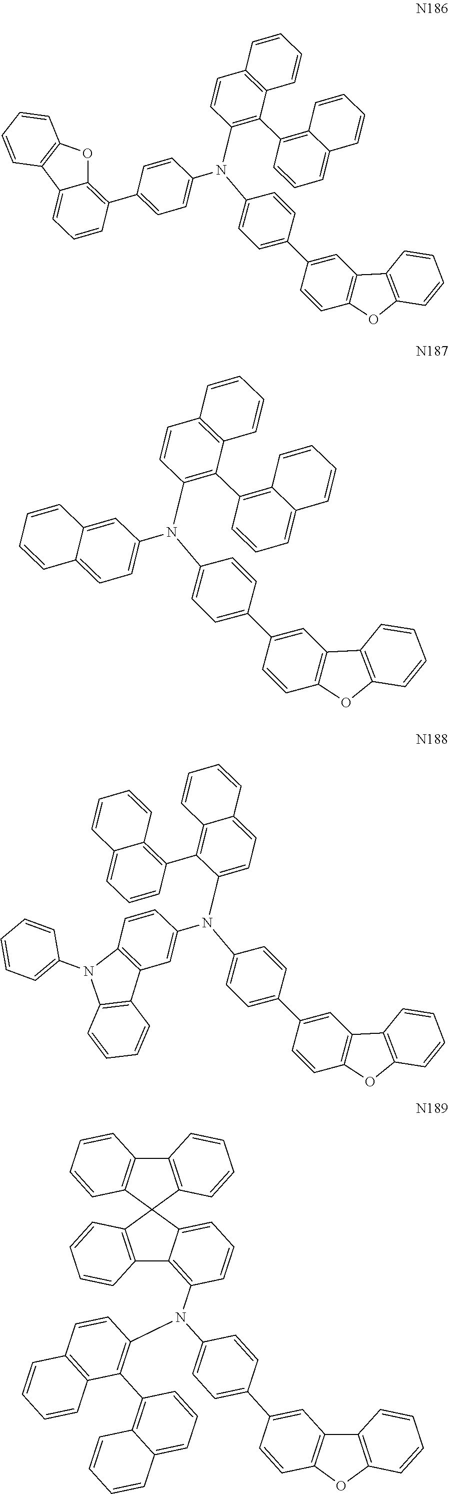 [SMILES: c1ccc2c(-c3c(N(c4ccc(-c5ccc6oc7ccccc7c6c5)cc4)c4ccc(-c5cccc6c5oc5ccccc56)cc4)ccc4ccccc34)cccc2c1 ; c1ccc2cc(N(c3ccc(-c4ccc5oc6ccccc6c5c4)cc3)c3ccc4ccccc4c3-c3cccc4ccccc34)ccc2c1 ; c1ccc(-n2c3ccccc3c3cc(N(c4ccc(-c5ccc6oc7ccccc7c6c5)cc4)c4ccc5ccccc5c4-c4cccc5ccccc45)ccc32)cc1 ; c1ccc2c(c1)-c1ccccc1C21c2ccccc2-c2c(N(c3ccc(-c4ccc5oc6ccccc6c5c4)cc3)c3ccc4ccccc4c3-c3cccc4ccccc34)cccc21]

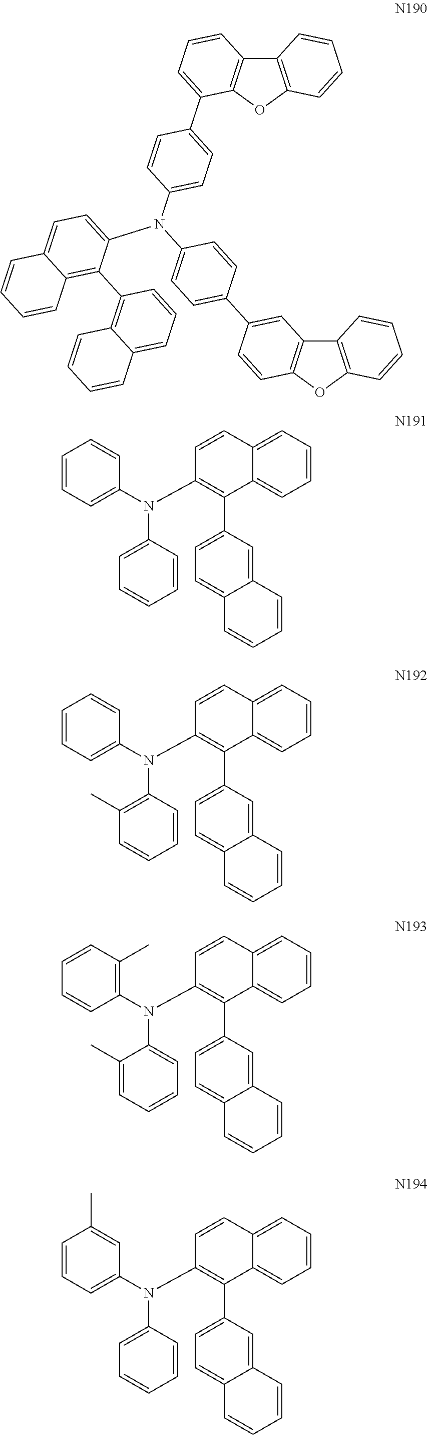

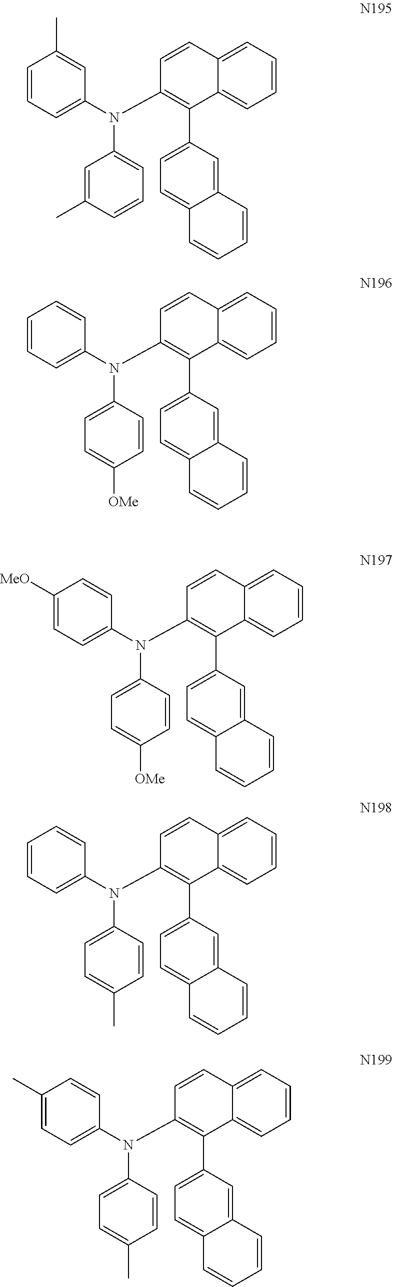

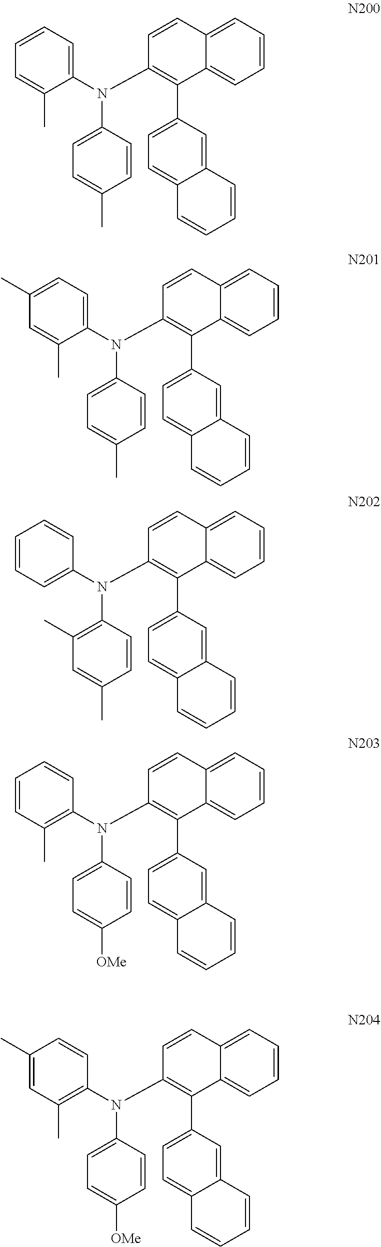

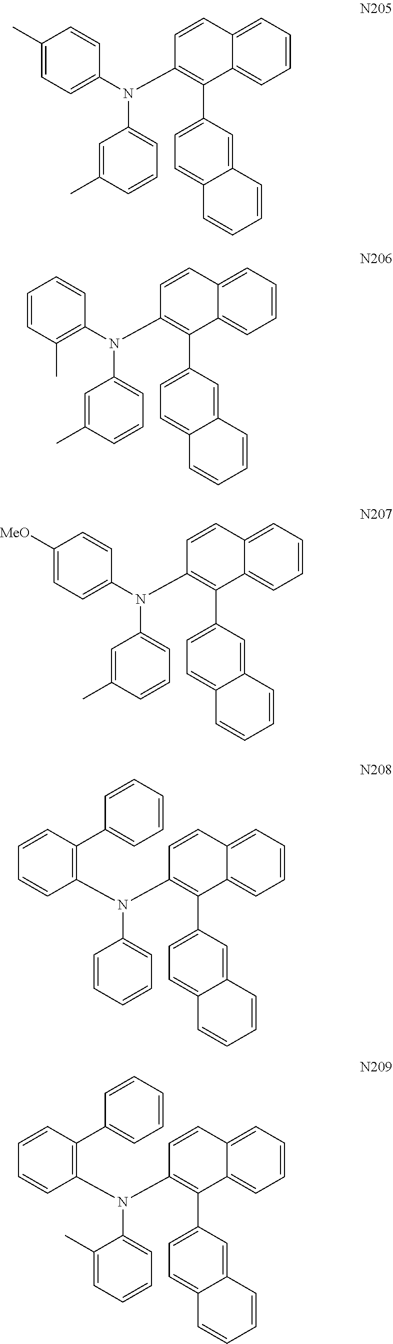

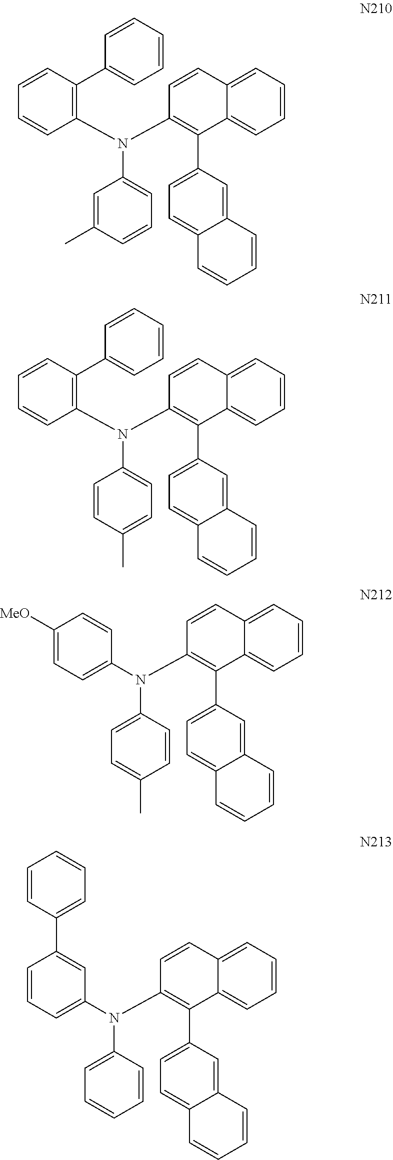

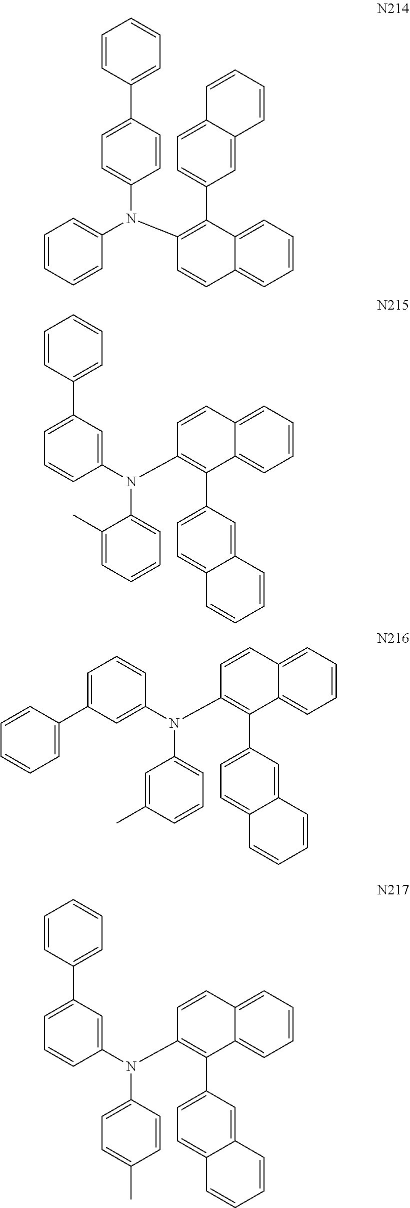

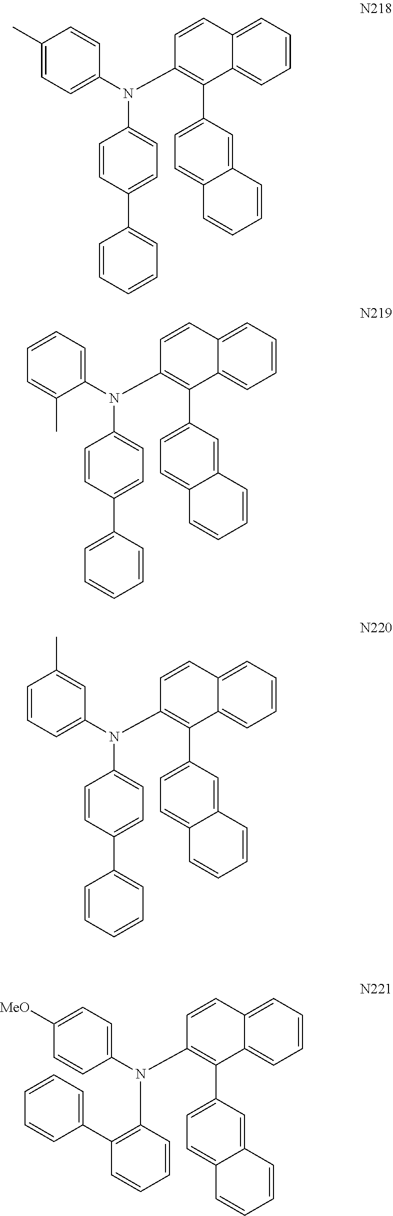 [SMILES: Cc1ccc(N(c2ccc(-c3ccccc3)cc2)c2ccc3ccccc3c2-c2ccc3ccccc3c2)cc1 ; Cc1ccccc1N(c1ccc(-c2ccccc2)cc1)c1ccc2ccccc2c1-c1ccc2ccccc2c1 ; Cc1cccc(N(c2ccc(-c3ccccc3)cc2)c2ccc3ccccc3c2-c2ccc3ccccc3c2)c1 ; COc1ccc(N(c2ccccc2-c2ccccc2)c2ccc3ccccc3c2-c2ccc3ccccc3c2)cc1]

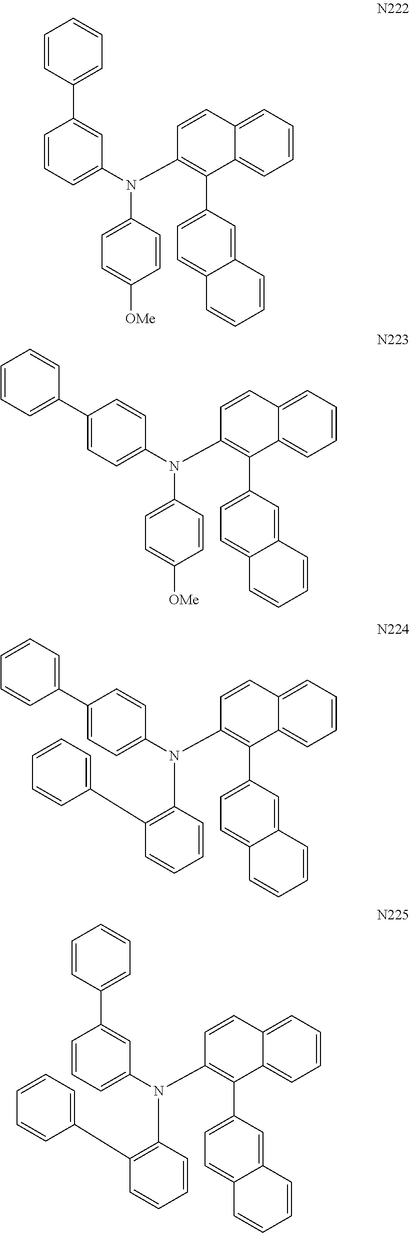 [SMILES: COc1ccc(N(c2cccc(-c3ccccc3)c2)c2ccc3ccccc3c2-c2ccc3ccccc3c2)cc1 ; COc1ccc(N(c2ccc(-c3ccccc3)cc2)c2ccc3ccccc3c2-c2ccc3ccccc3c2)cc1 ; c1ccc(-c2ccc(N(c3ccccc3-c3ccccc3)c3ccc4ccccc4c3-c3ccc4ccccc4c3)cc2)cc1 ; c1ccc(-c2cccc(N(c3ccccc3-c3ccccc3)c3ccc4ccccc4c3-c3ccc4ccccc4c3)c2)cc1]

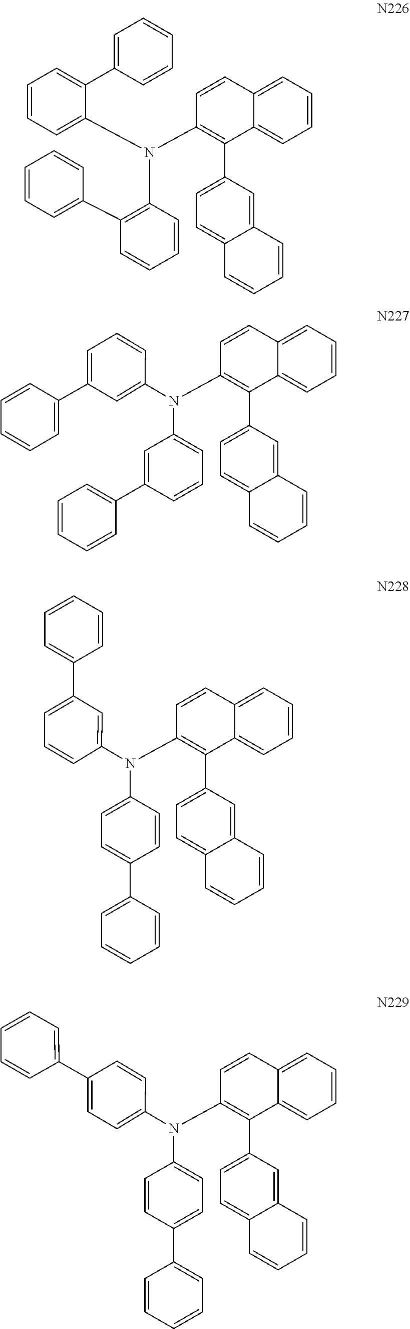

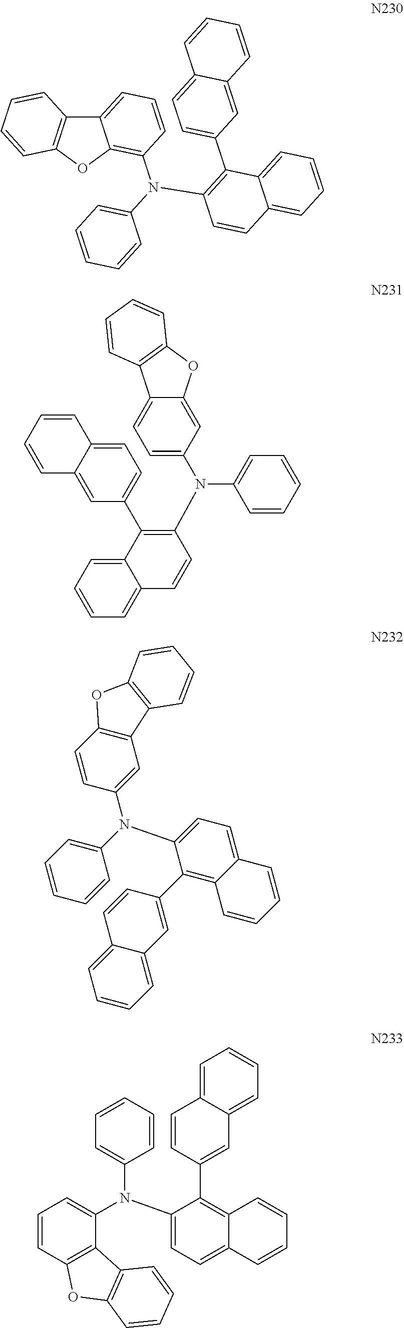

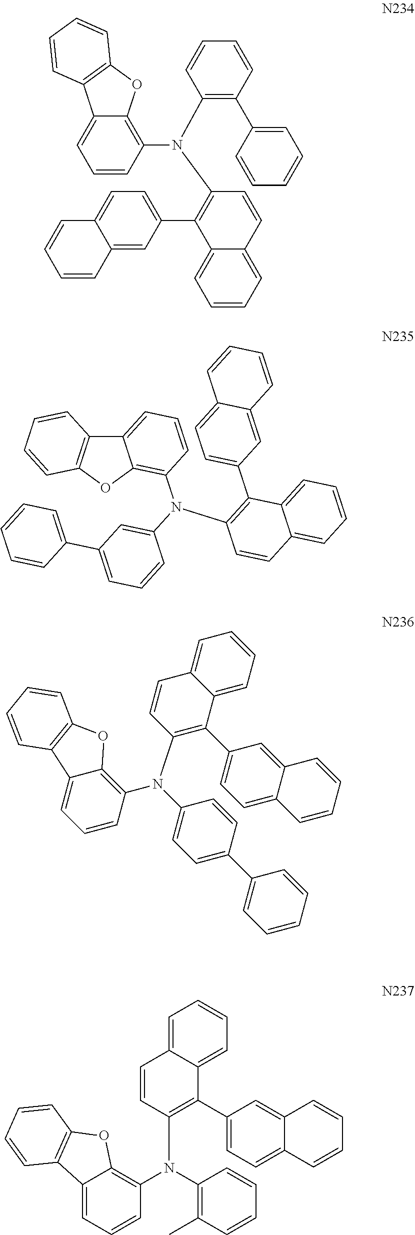

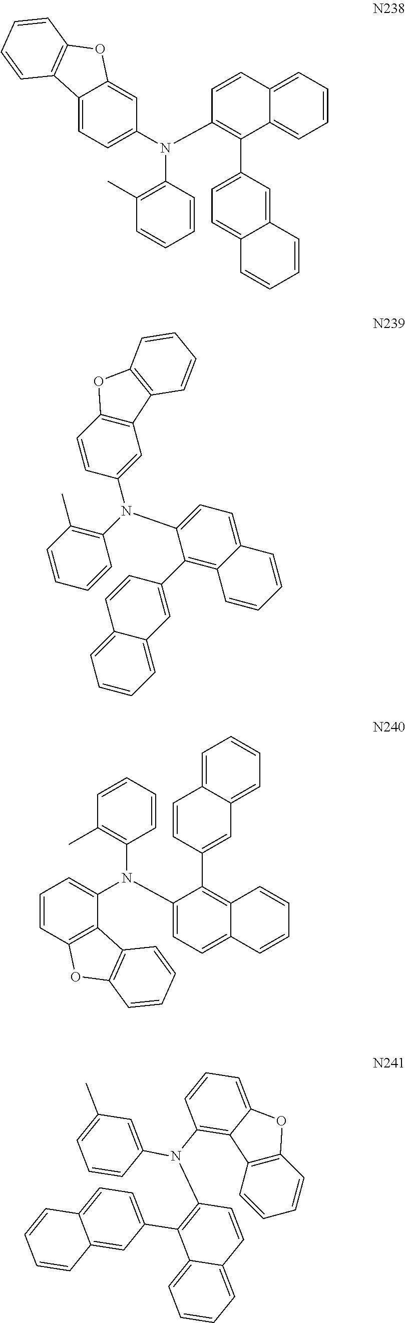 [SMILES: Cc1ccccc1N(c1ccc2c(c1)oc1ccccc12)c1ccc2ccccc2c1-c1ccc2ccccc2c1 ; Cc1ccccc1N(c1ccc2oc3ccccc3c2c1)c1ccc2ccccc2c1-c1ccc2ccccc2c1 ; Cc1ccccc1N(c1ccc2ccccc2c1-c1ccc2ccccc2c1)c1cccc2oc3ccccc3c12 ; Cc1cccc(N(c2ccc3ccccc3c2-c2ccc3ccccc3c2)c2cccc3oc4ccccc4c23)c1]

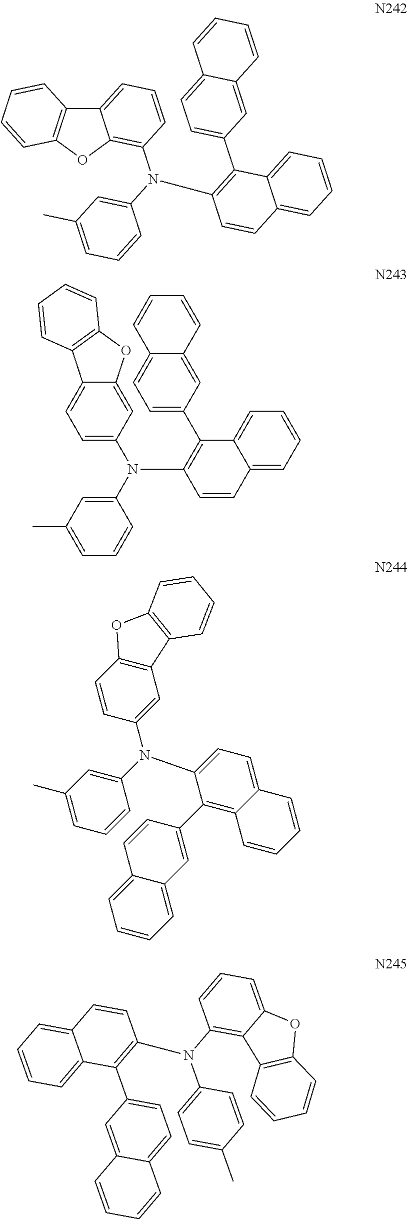

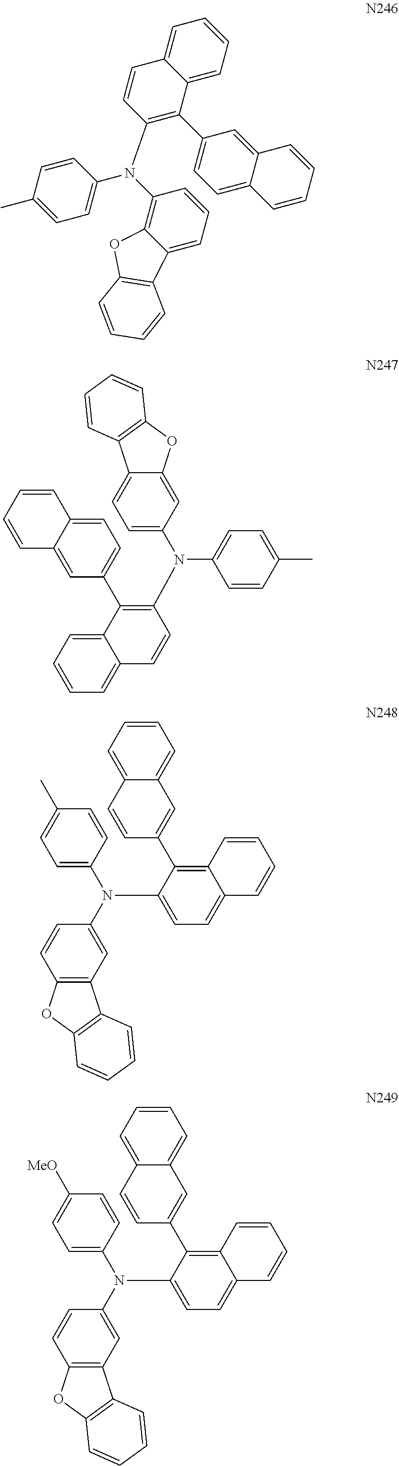

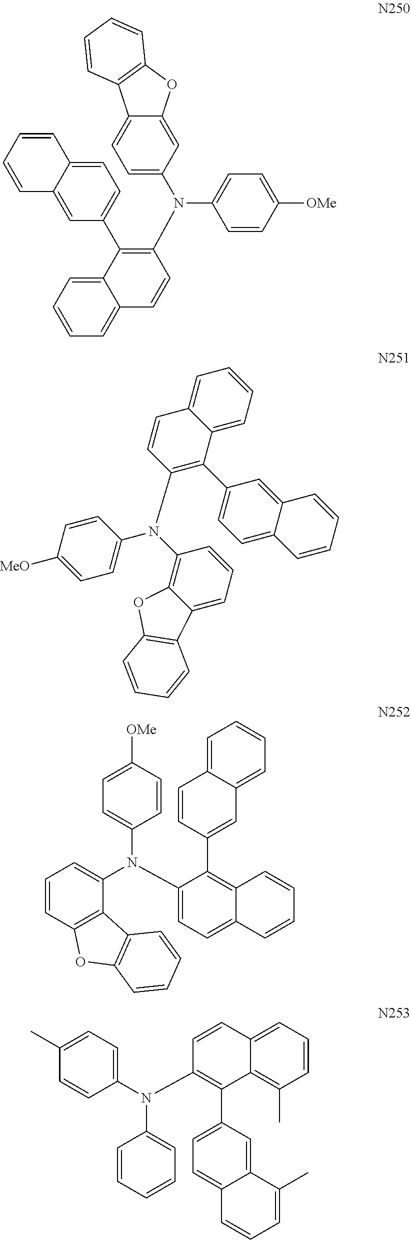

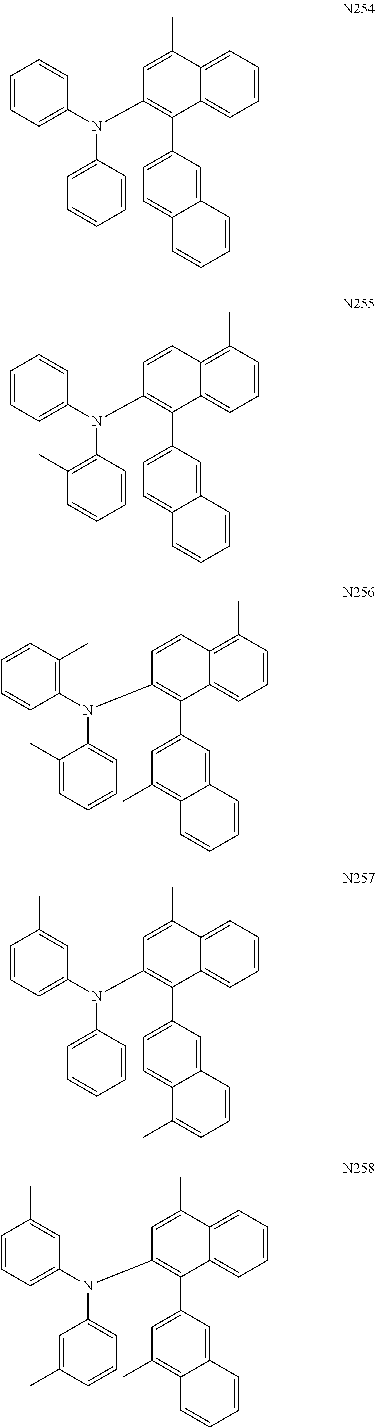

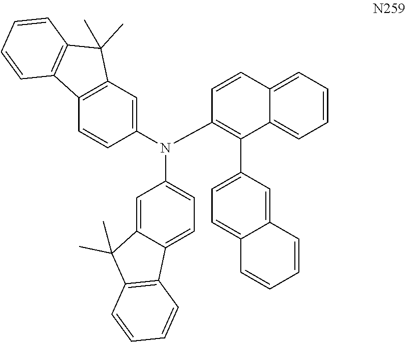 [SMILES: CC1(C)c2ccccc2-c2ccc(N(c3ccc4c(c3)C(C)(C)c3ccccc3-4)c3ccc4ccccc4c3-c3ccc4ccccc4c3)cc21]

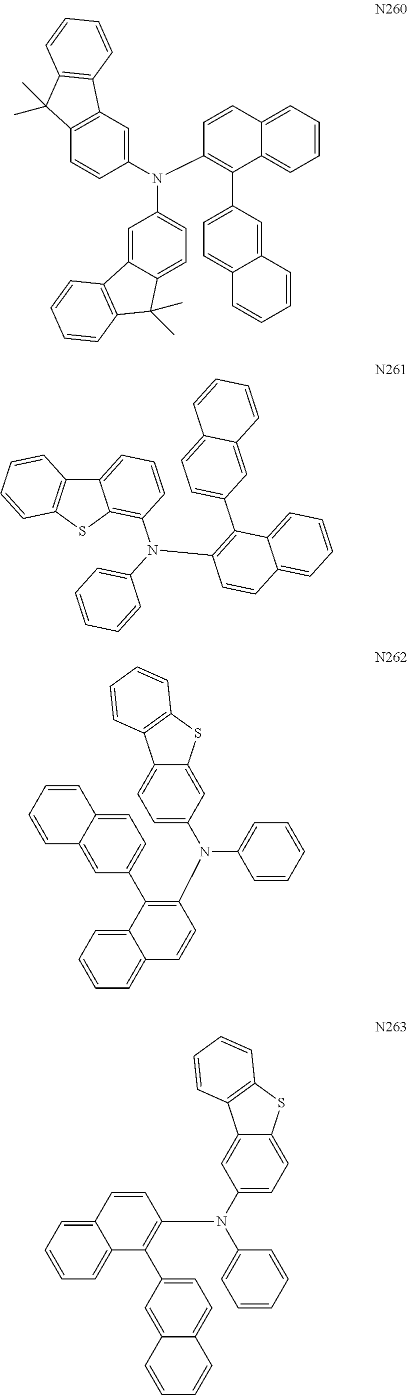

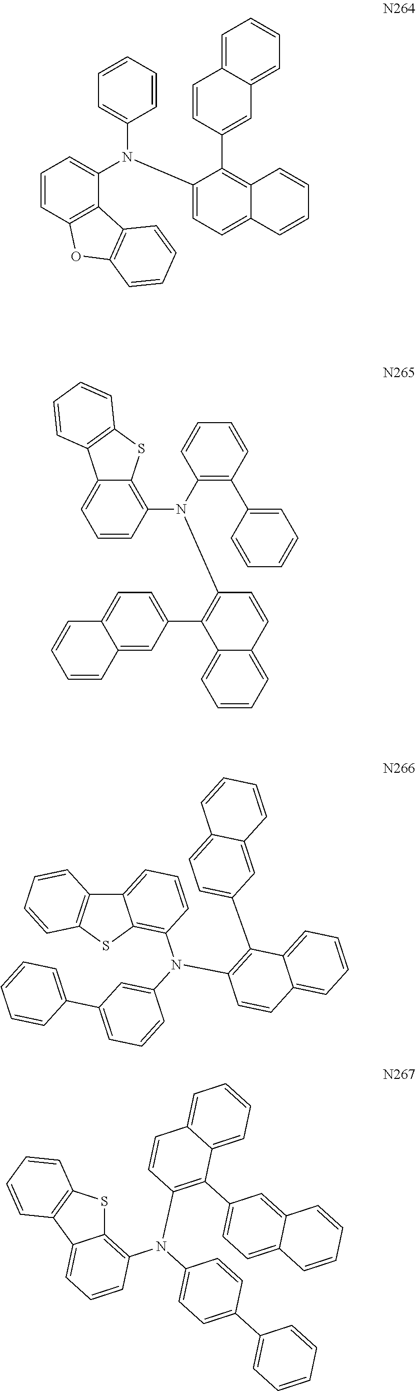 [SMILES: c1ccc(N(c2ccc3ccccc3c2-c2ccc3ccccc3c2)c2cccc3oc4ccccc4c23)cc1 ; c1ccc(-c2ccccc2N(c2ccc3ccccc3c2-c2ccc3ccccc3c2)c2cccc3c2sc2ccccc23)cc1 ; c1ccc(-c2cccc(N(c3ccc4ccccc4c3-c3ccc4ccccc4c3)c3cccc4c3sc3ccccc34)c2)cc1 ; c1ccc(-c2ccc(N(c3ccc4ccccc4c3-c3ccc4ccccc4c3)c3cccc4c3sc3ccccc34)cc2)cc1]

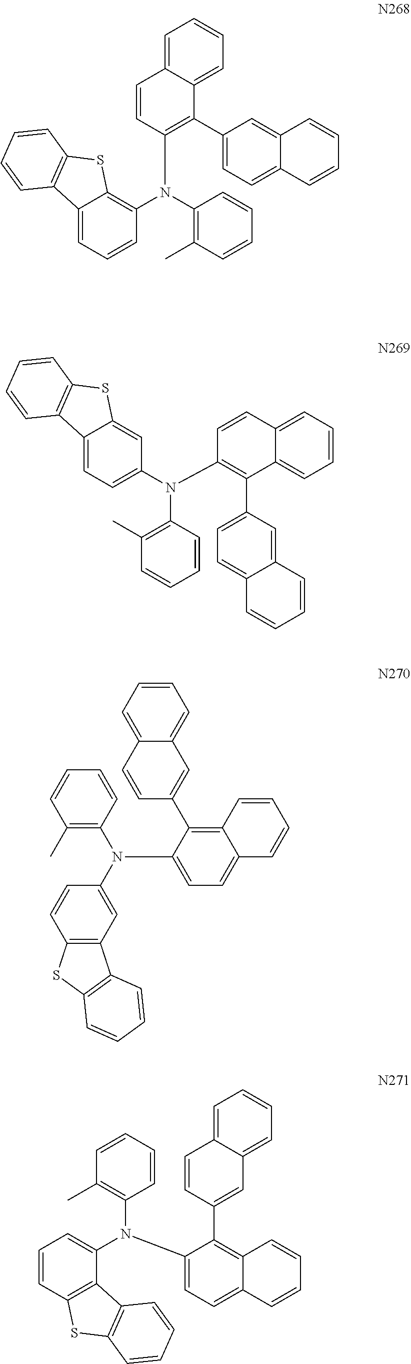 [SMILES: Cc1ccccc1N(c1ccc2ccccc2c1-c1ccc2ccccc2c1)c1cccc2c1sc1ccccc12 ; Cc1ccccc1N(c1ccc2c(c1)sc1ccccc12)c1ccc2ccccc2c1-c1ccc2ccccc2c1 ; Cc1ccccc1N(c1ccc2sc3ccccc3c2c1)c1ccc2ccccc2c1-c1ccc2ccccc2c1 ; Cc1ccccc1N(c1ccc2ccccc2c1-c1ccc2ccccc2c1)c1cccc2sc3ccccc3c12]

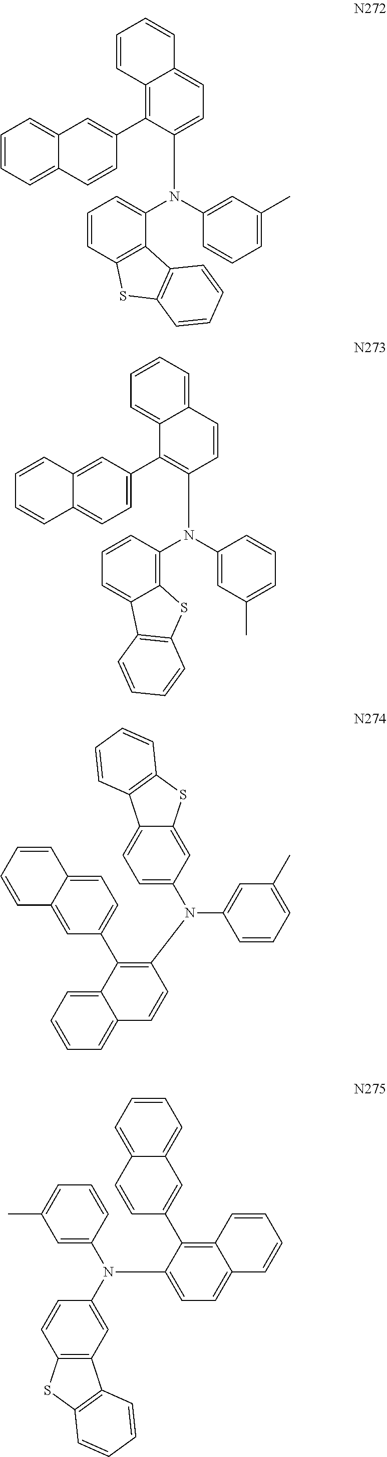

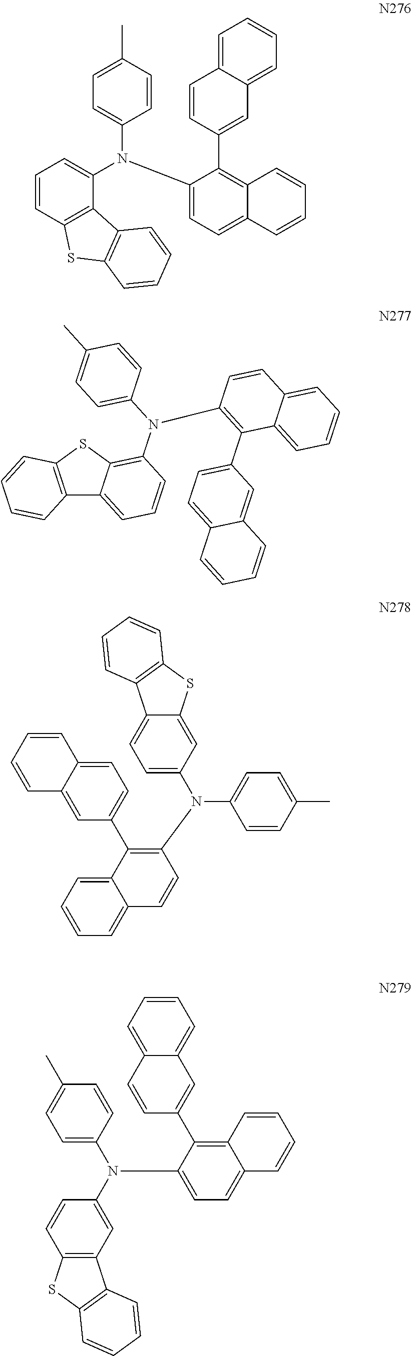 [SMILES: Cc1ccc(N(c2ccc3ccccc3c2-c2ccc3ccccc3c2)c2cccc3sc4ccccc4c23)cc1 ; Cc1ccc(N(c2ccc3ccccc3c2-c2ccc3ccccc3c2)c2cccc3c2sc2ccccc23)cc1 ; Cc1ccc(N(c2ccc3c(c2)sc2ccccc23)c2ccc3ccccc3c2-c2ccc3ccccc3c2)cc1 ; Cc1ccc(N(c2ccc3sc4ccccc4c3c2)c2ccc3ccccc3c2-c2ccc3ccccc3c2)cc1]

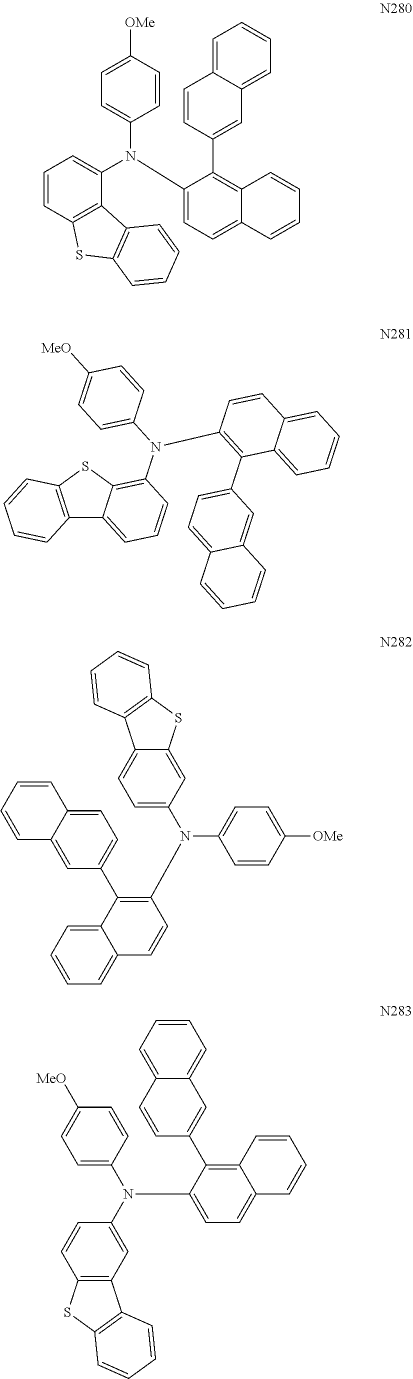 [SMILES: COc1ccc(N(c2ccc3ccccc3c2-c2ccc3ccccc3c2)c2cccc3sc4ccccc4c23)cc1 ; COc1ccc(N(c2ccc3ccccc3c2-c2ccc3ccccc3c2)c2cccc3c2sc2ccccc23)cc1 ; COc1ccc(N(c2ccc3c(c2)sc2ccccc23)c2ccc3ccccc3c2-c2ccc3ccccc3c2)cc1 ; COc1ccc(N(c2ccc3sc4ccccc4c3c2)c2ccc3ccccc3c2-c2ccc3ccccc3c2)cc1]

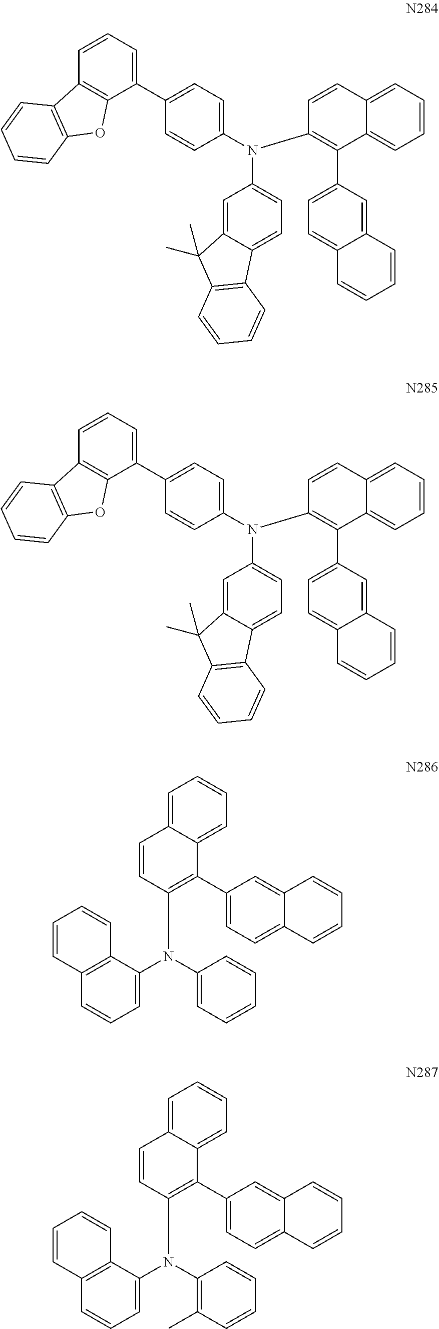 [SMILES: CC1(C)c2ccccc2-c2ccc(N(c3ccc(-c4cccc5c4oc4ccccc45)cc3)c3ccc4ccccc4c3-c3ccc4ccccc4c3)cc21 ; CC1(C)c2ccccc2-c2ccc(N(c3ccc(-c4cccc5c4oc4ccccc45)cc3)c3ccc4ccccc4c3-c3ccc4ccccc4c3)cc21 ; c1ccc(N(c2ccc3ccccc3c2-c2ccc3ccccc3c2)c2cccc3ccccc23)cc1 ; Cc1ccccc1N(c1ccc2ccccc2c1-c1ccc2ccccc2c1)c1cccc2ccccc12]

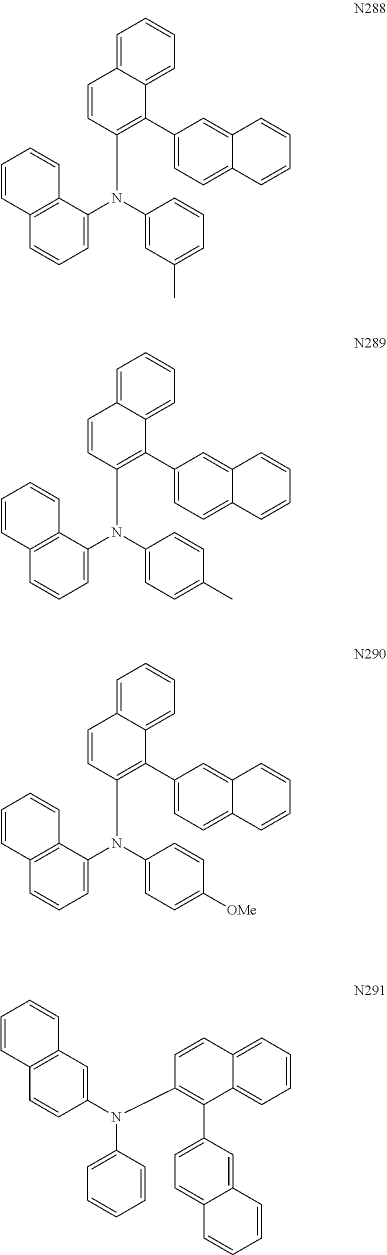 [SMILES: Cc1cccc(N(c2ccc3ccccc3c2-c2ccc3ccccc3c2)c2cccc3ccccc23)c1 ; Cc1ccc(N(c2ccc3ccccc3c2-c2ccc3ccccc3c2)c2cccc3ccccc23)cc1 ; COc1ccc(N(c2ccc3ccccc3c2-c2ccc3ccccc3c2)c2cccc3ccccc23)cc1 ; c1ccc(N(c2ccc3ccccc3c2)c2ccc3ccccc3c2-c2ccc3ccccc3c2)cc1]

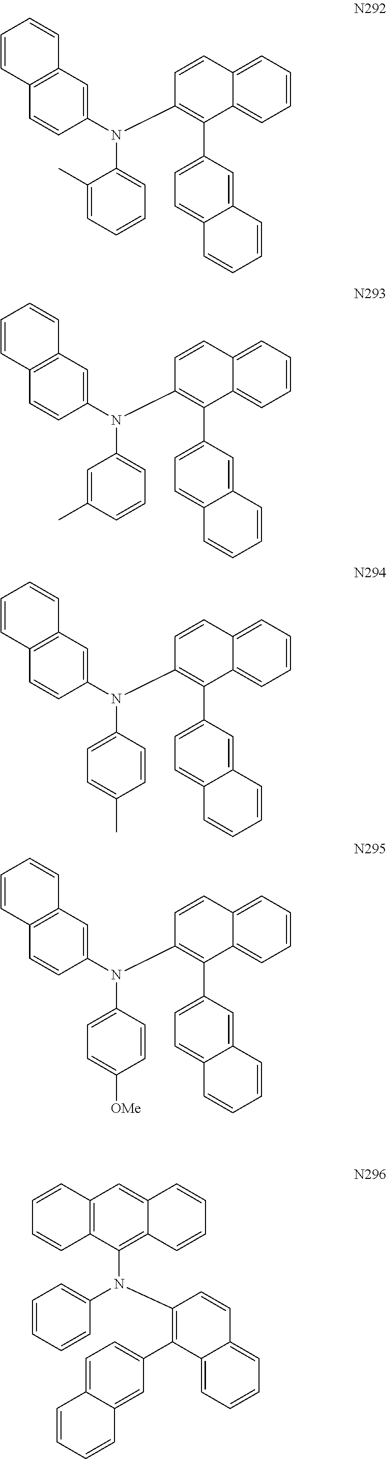 [SMILES: Cc1ccccc1N(c1ccc2ccccc2c1)c1ccc2ccccc2c1-c1ccc2ccccc2c1 ; Cc1cccc(N(c2ccc3ccccc3c2)c2ccc3ccccc3c2-c2ccc3ccccc3c2)c1 ; Cc1ccc(N(c2ccc3ccccc3c2)c2ccc3ccccc3c2-c2ccc3ccccc3c2)cc1 ; COc1ccc(N(c2ccc3ccccc3c2)c2ccc3ccccc3c2-c2ccc3ccccc3c2)cc1 ; c1ccc(N(c2ccc3ccccc3c2-c2ccc3ccccc3c2)c2c3ccccc3cc3ccccc23)cc1]

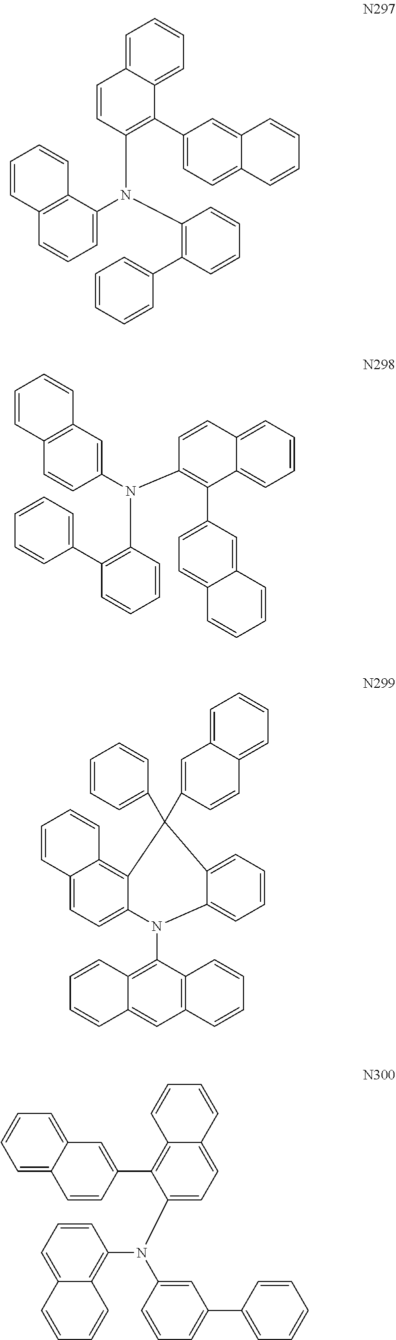 [SMILES: c1ccc(-c2ccccc2N(c2ccc3ccccc3c2-c2ccc3ccccc3c2)c2cccc3ccccc23)cc1 ; c1ccc(-c2ccccc2N(c2ccc3ccccc3c2)c2ccc3ccccc3c2-c2ccc3ccccc3c2)cc1 ; c1ccc(C2(c3ccc4ccccc4c3)c3ccccc3N(c3c4ccccc4cc4ccccc34)c3ccc4ccccc4c32)cc1 ; c1ccc(-c2cccc(N(c3ccc4ccccc4c3-c3ccc4ccccc4c3)c3cccc4ccccc34)c2)cc1]

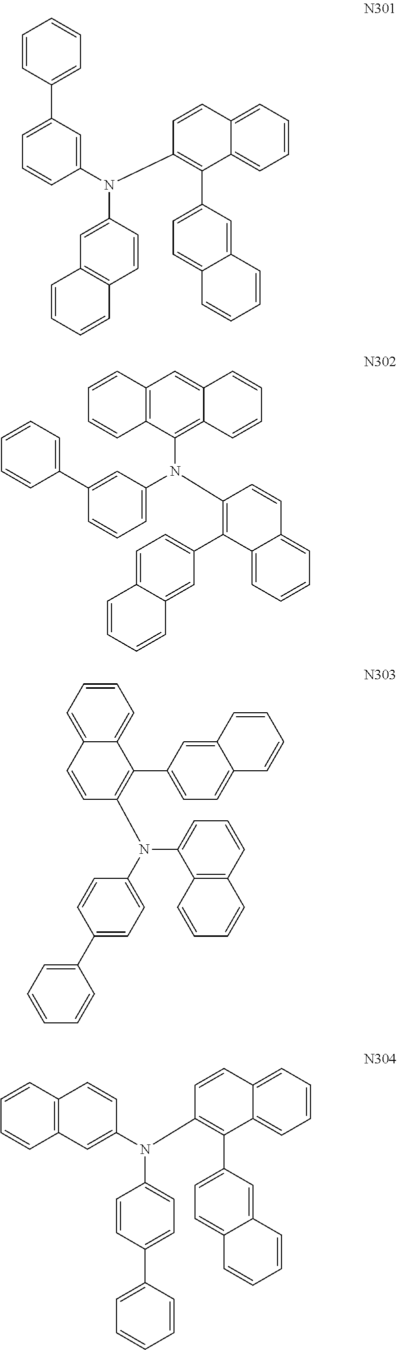

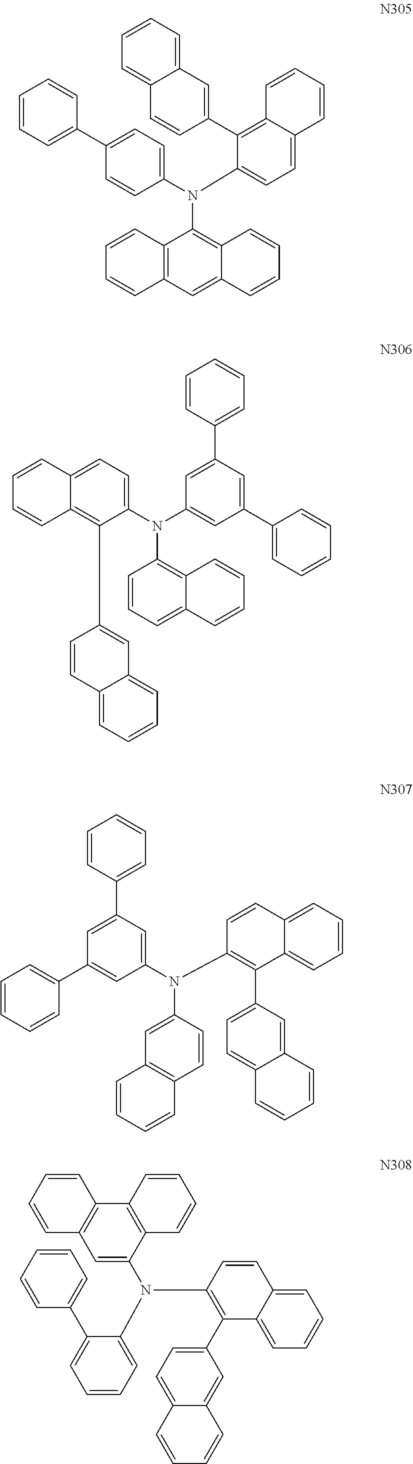

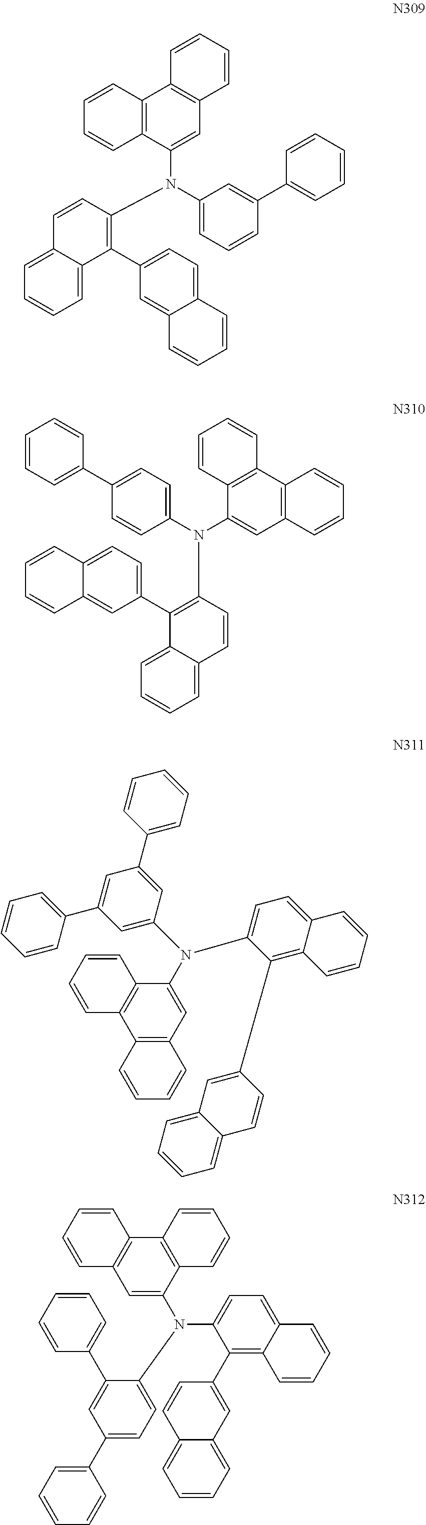

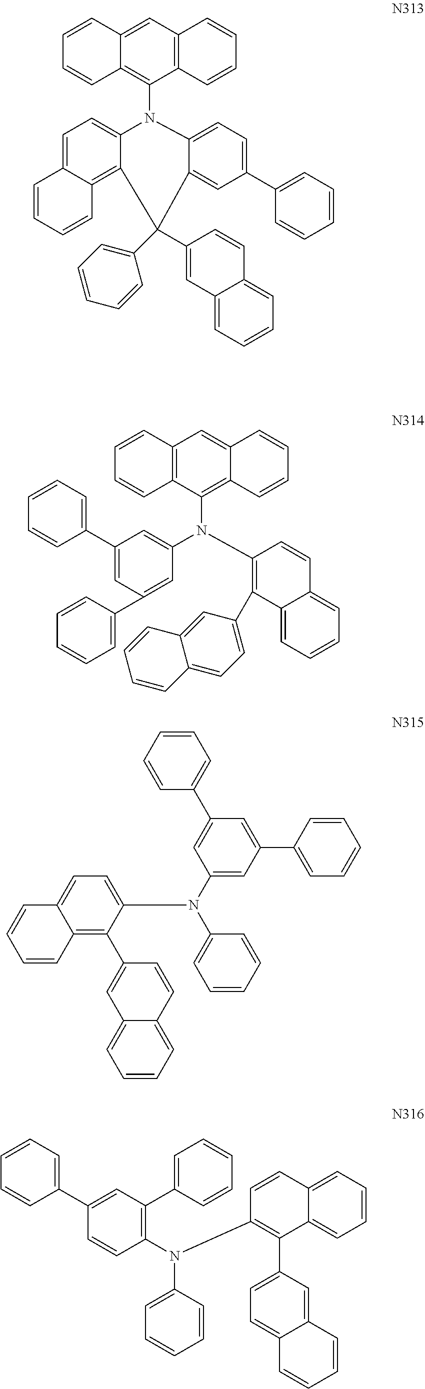

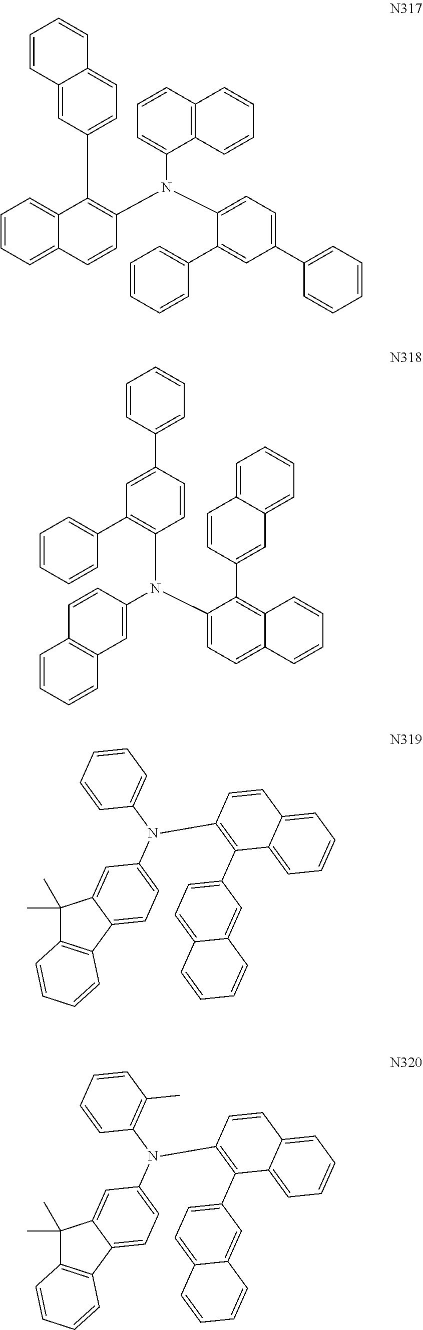 [SMILES: c1ccc(-c2ccc(N(c3ccc4ccccc4c3-c3ccc4ccccc4c3)c3cccc4ccccc34)c(-c3ccccc3)c2)cc1 ; c1ccc(-c2ccc(N(c3ccc4ccccc4c3)c3ccc4ccccc4c3-c3ccc4ccccc4c3)c(-c3ccccc3)c2)cc1 ; CC1(C)c2ccccc2-c2ccc(N(c3ccccc3)c3ccc4ccccc4c3-c3ccc4ccccc4c3)cc21 ; Cc1ccccc1N(c1ccc2c(c1)C(C)(C)c1ccccc1-2)c1ccc2ccccc2c1-c1ccc2ccccc2c1]

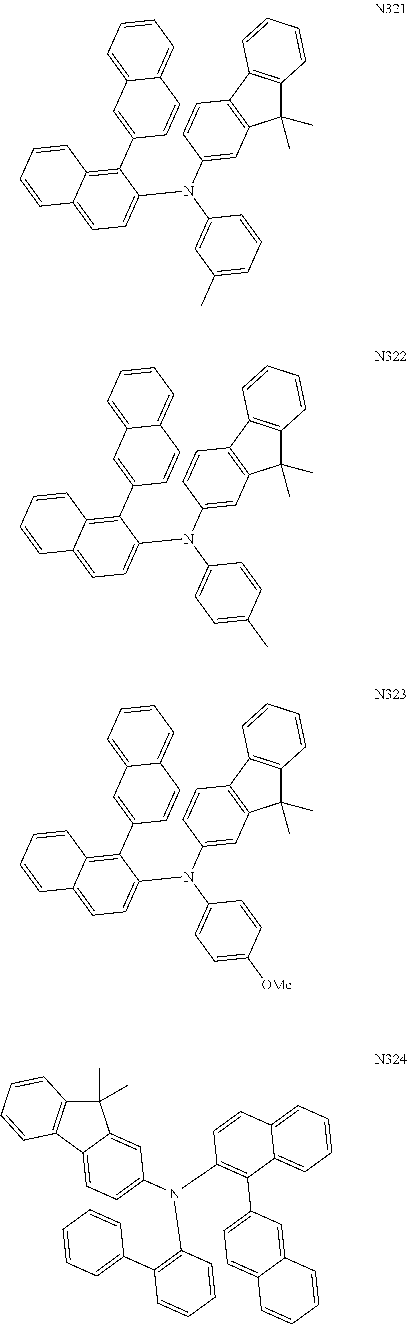 [SMILES: Cc1cccc(N(c2ccc3c(c2)C(C)(C)c2ccccc2-3)c2ccc3ccccc3c2-c2ccc3ccccc3c2)c1 ; Cc1ccc(N(c2ccc3c(c2)C(C)(C)c2ccccc2-3)c2ccc3ccccc3c2-c2ccc3ccccc3c2)cc1 ; COc1ccc(N(c2ccc3c(c2)C(C)(C)c2ccccc2-3)c2ccc3ccccc3c2-c2ccc3ccccc3c2)cc1 ; CC1(C)c2ccccc2-c2ccc(N(c3ccccc3-c3ccccc3)c3ccc4ccccc4c3-c3ccc4ccccc4c3)cc21]

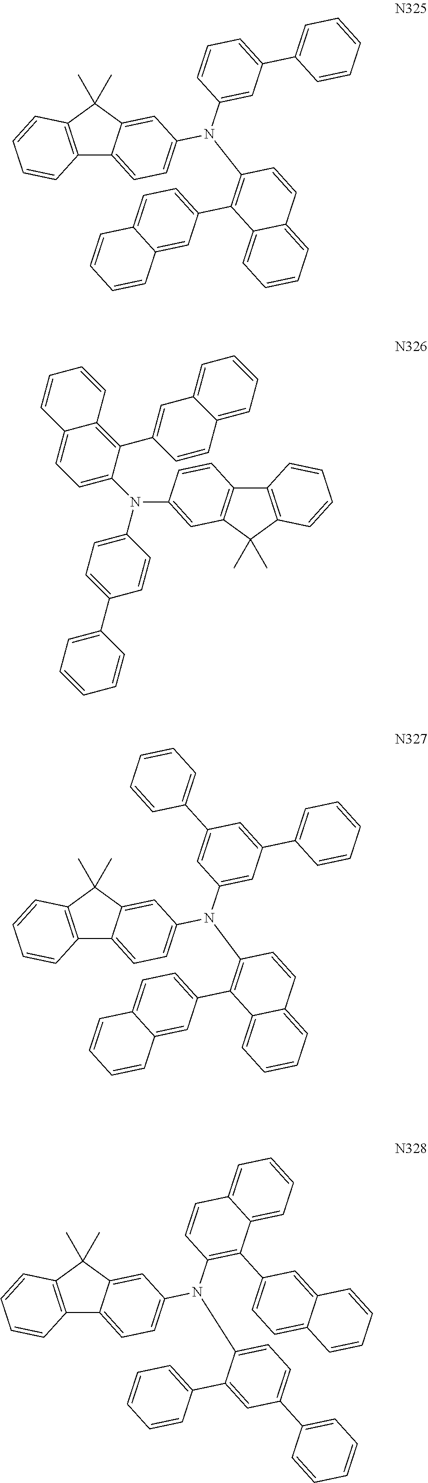 [SMILES: CC1(C)c2ccccc2-c2ccc(N(c3cccc(-c4ccccc4)c3)c3ccc4ccccc4c3-c3ccc4ccccc4c3)cc21 ; CC1(C)c2ccccc2-c2ccc(N(c3ccc(-c4ccccc4)cc3)c3ccc4ccccc4c3-c3ccc4ccccc4c3)cc21 ; CC1(C)c2ccccc2-c2ccc(N(c3cc(-c4ccccc4)cc(-c4ccccc4)c3)c3ccc4ccccc4c3-c3ccc4ccccc4c3)cc21 ; CC1(C)c2ccccc2-c2ccc(N(c3ccc(-c4ccccc4)cc3-c3ccccc3)c3ccc4ccccc4c3-c3ccc4ccccc4c3)cc21]

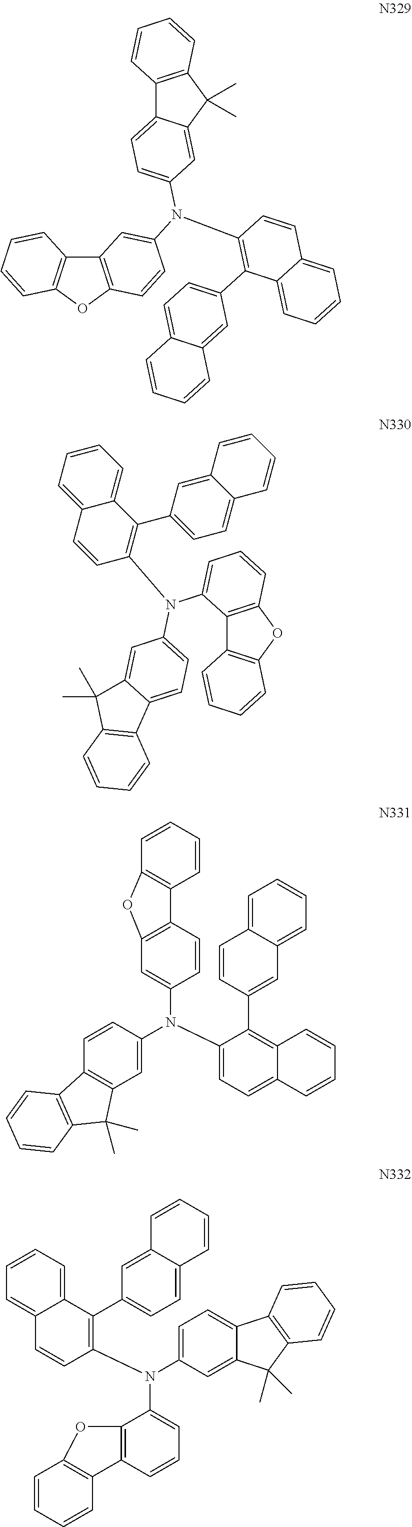 [SMILES: CC1(C)c2ccccc2-c2ccc(N(c3ccc4oc5ccccc5c4c3)c3ccc4ccccc4c3-c3ccc4ccccc4c3)cc21 ; CC1(C)c2ccccc2-c2ccc(N(c3ccc4ccccc4c3-c3ccc4ccccc4c3)c3cccc4oc5ccccc5c34)cc21 ; CC1(C)c2ccccc2-c2ccc(N(c3ccc4c(c3)oc3ccccc34)c3ccc4ccccc4c3-c3ccc4ccccc4c3)cc21 ; CC1(C)c2ccccc2-c2ccc(N(c3ccc4ccccc4c3-c3ccc4ccccc4c3)c3cccc4c3oc3ccccc34)cc21]

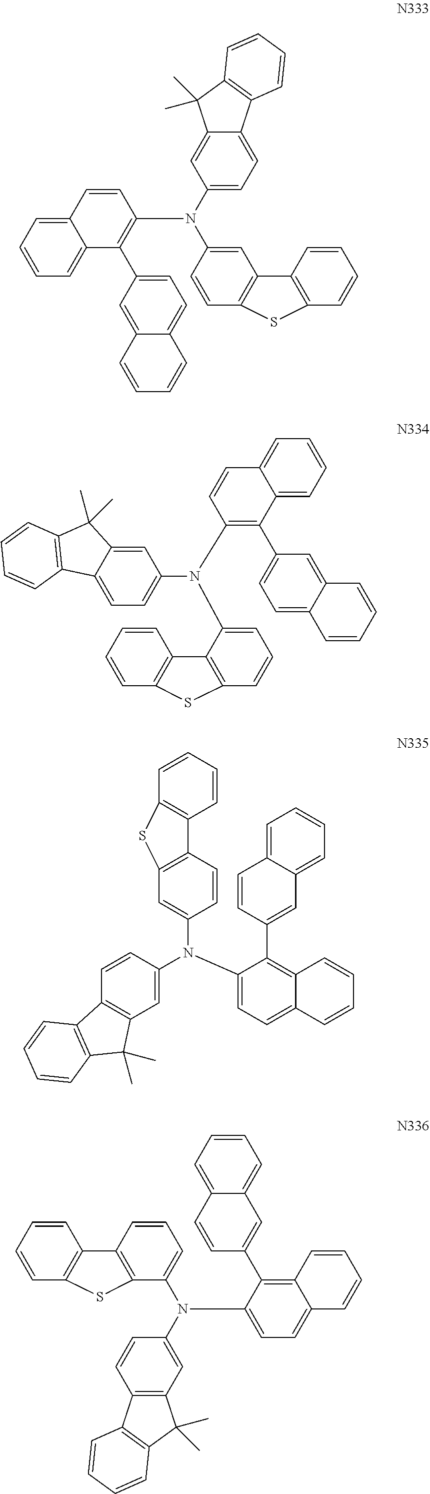 [SMILES: CC1(C)c2ccccc2-c2ccc(N(c3ccc4sc5ccccc5c4c3)c3ccc4ccccc4c3-c3ccc4ccccc4c3)cc21 ; CC1(C)c2ccccc2-c2ccc(N(c3ccc4ccccc4c3-c3ccc4ccccc4c3)c3cccc4sc5ccccc5c34)cc21 ; CC1(C)c2ccccc2-c2ccc(N(c3ccc4c(c3)sc3ccccc34)c3ccc4ccccc4c3-c3ccc4ccccc4c3)cc21 ; CC1(C)c2ccccc2-c2ccc(N(c3ccc4ccccc4c3-c3ccc4ccccc4c3)c3cccc4c3sc3ccccc34)cc21]

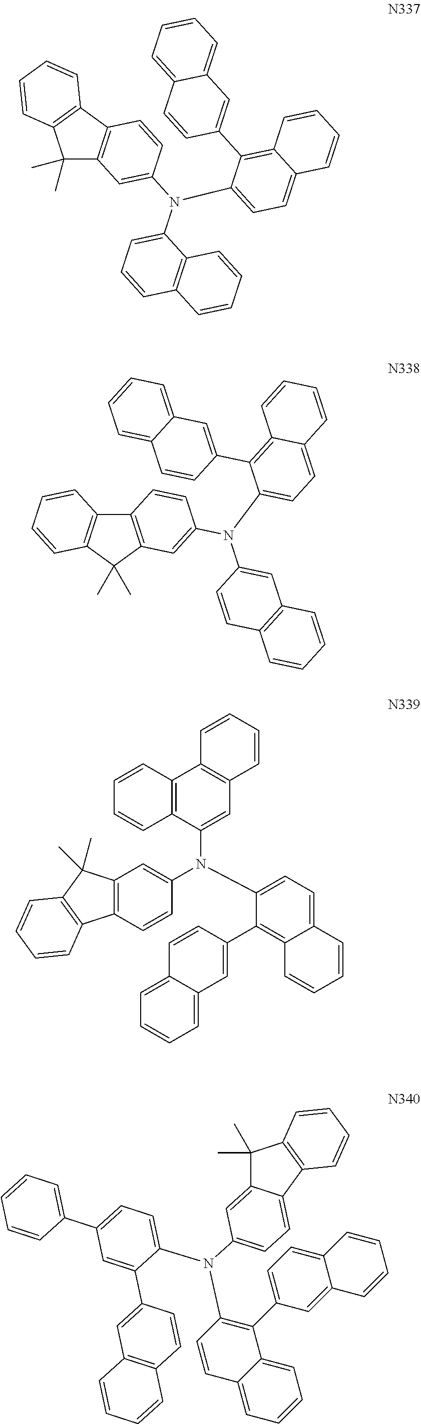 [SMILES: CC1(C)c2ccccc2-c2ccc(N(c3ccc4ccccc4c3-c3ccc4ccccc4c3)c3cccc4ccccc34)cc21 ; CC1(C)c2ccccc2-c2ccc(N(c3ccc4ccccc4c3)c3ccc4ccccc4c3-c3ccc4ccccc4c3)cc21 ; CC1(C)c2ccccc2-c2ccc(N(c3ccc4ccccc4c3-c3ccc4ccccc4c3)c3cc4ccccc4c4ccccc34)cc21 ; CC1(C)c2ccccc2-c2ccc(N(c3ccc(-c4ccccc4)cc3-c3ccc4ccccc4c3)c3ccc4ccccc4c3-c3ccc4ccccc4c3)cc21]

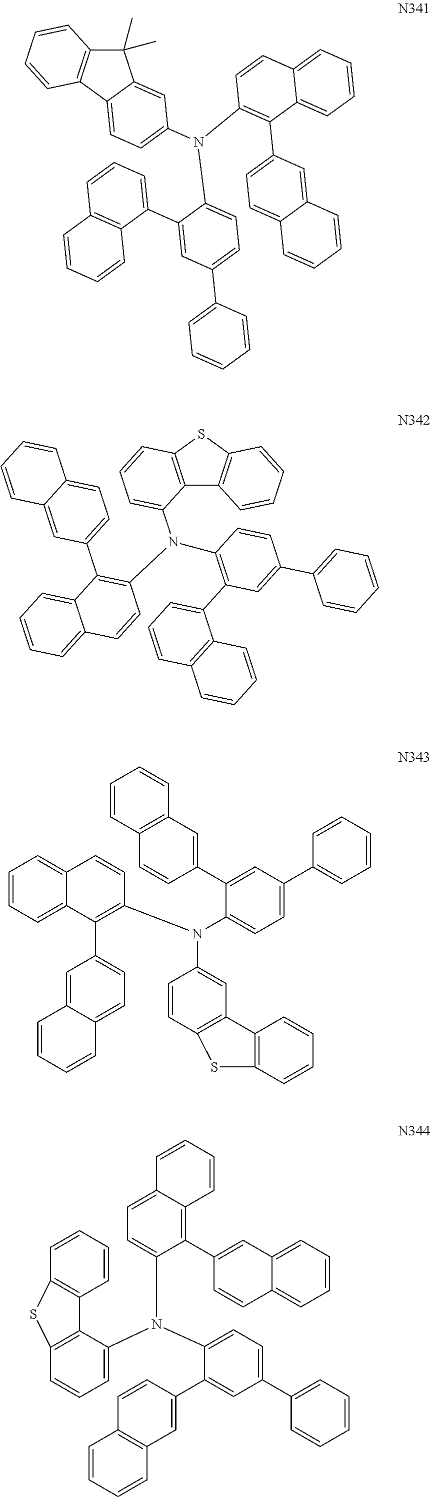 [SMILES: CC1(C)c2ccccc2-c2ccc(N(c3ccc(-c4ccccc4)cc3-c3cccc4ccccc34)c3ccc4ccccc4c3-c3ccc4ccccc4c3)cc21 ; c1ccc(-c2ccc(N(c3ccc4ccccc4c3-c3ccc4ccccc4c3)c3cccc4sc5ccccc5c34)c(-c3cccc4ccccc34)c2)cc1 ; c1ccc(-c2ccc(N(c3ccc4sc5ccccc5c4c3)c3ccc4ccccc4c3-c3ccc4ccccc4c3)c(-c3ccc4ccccc4c3)c2)cc1 ; c1ccc(-c2ccc(N(c3ccc4ccccc4c3-c3ccc4ccccc4c3)c3cccc4sc5ccccc5c34)c(-c3ccc4ccccc4c3)c2)cc1]

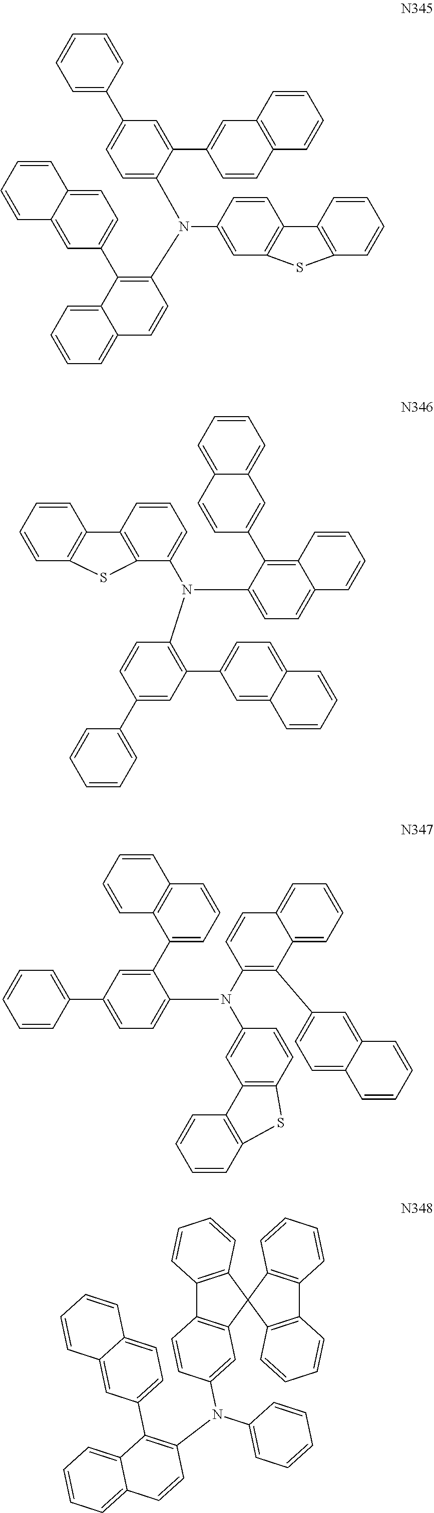 [SMILES: c1ccc(-c2ccc(N(c3ccc4c(c3)sc3ccccc34)c3ccc4ccccc4c3-c3ccc4ccccc4c3)c(-c3ccc4ccccc4c3)c2)cc1 ; c1ccc(-c2ccc(N(c3ccc4ccccc4c3-c3ccc4ccccc4c3)c3cccc4c3sc3ccccc34)c(-c3ccc4ccccc4c3)c2)cc1 ; c1ccc(-c2ccc(N(c3ccc4sc5ccccc5c4c3)c3ccc4ccccc4c3-c3ccc4ccccc4c3)c(-c3cccc4ccccc34)c2)cc1 ; c1ccc(N(c2ccc3c(c2)C2(c4ccccc4-c4ccccc42)c2ccccc2-3)c2ccc3ccccc3c2-c2ccc3ccccc3c2)cc1]

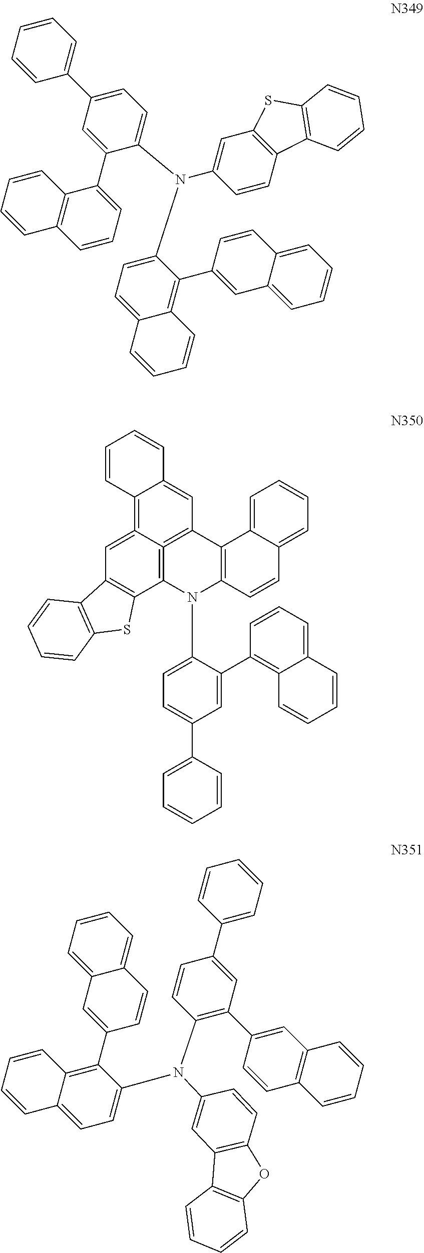

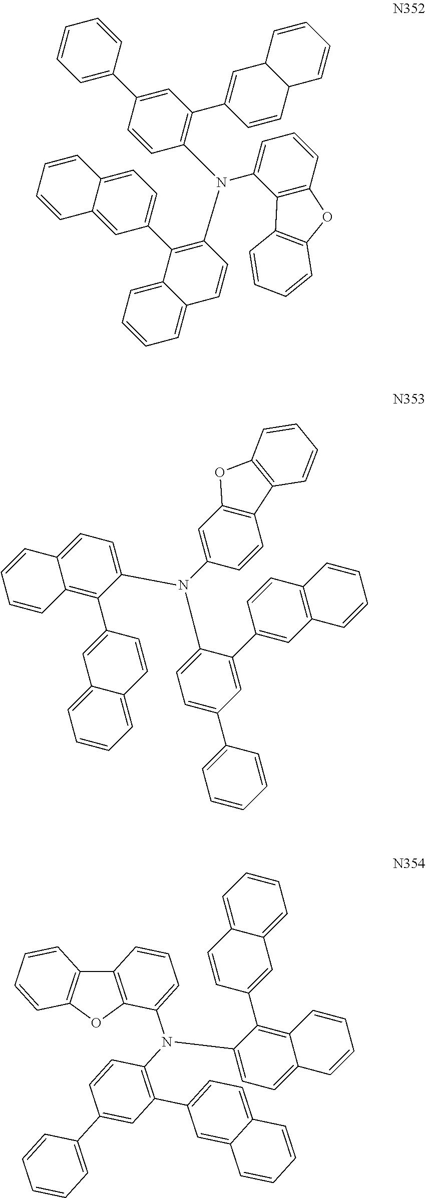 [SMILES: C1=CC2C=CC(c3cc(-c4ccccc4)ccc3N(c3ccc4ccccc4c3-c3ccc4ccccc4c3)c3cccc4oc5ccccc5c34)=CC2C=C1 ; c1ccc(-c2ccc(N(c3ccc4c(c3)oc3ccccc34)c3ccc4ccccc4c3-c3ccc4ccccc4c3)c(-c3ccc4ccccc4c3)c2)cc1 ; c1ccc(-c2ccc(N(c3ccc4ccccc4c3-c3ccc4ccccc4c3)c3cccc4c3oc3ccccc34)c(-c3ccc4ccccc4c3)c2)cc1]

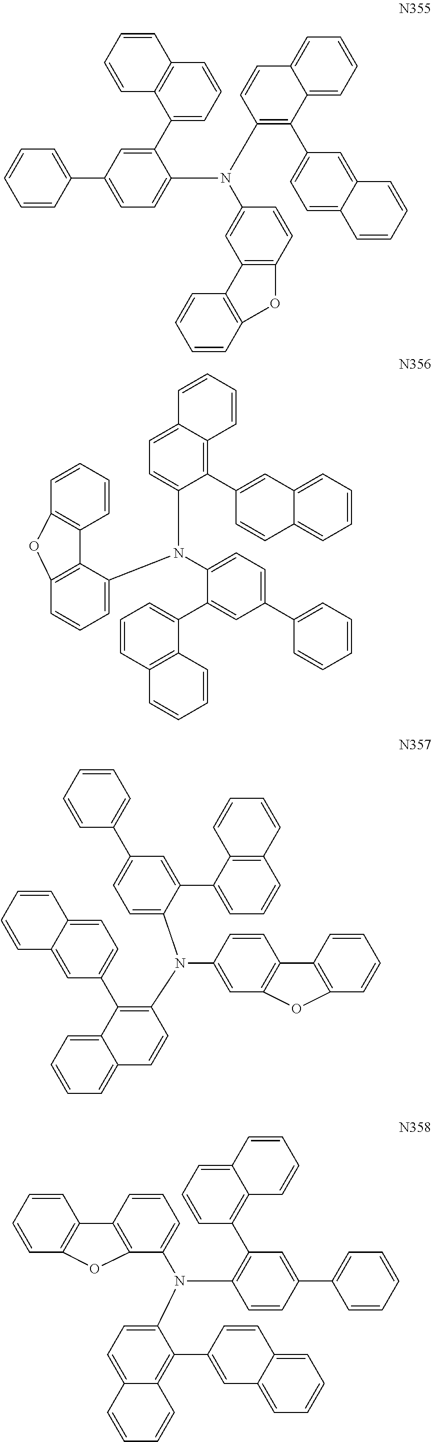

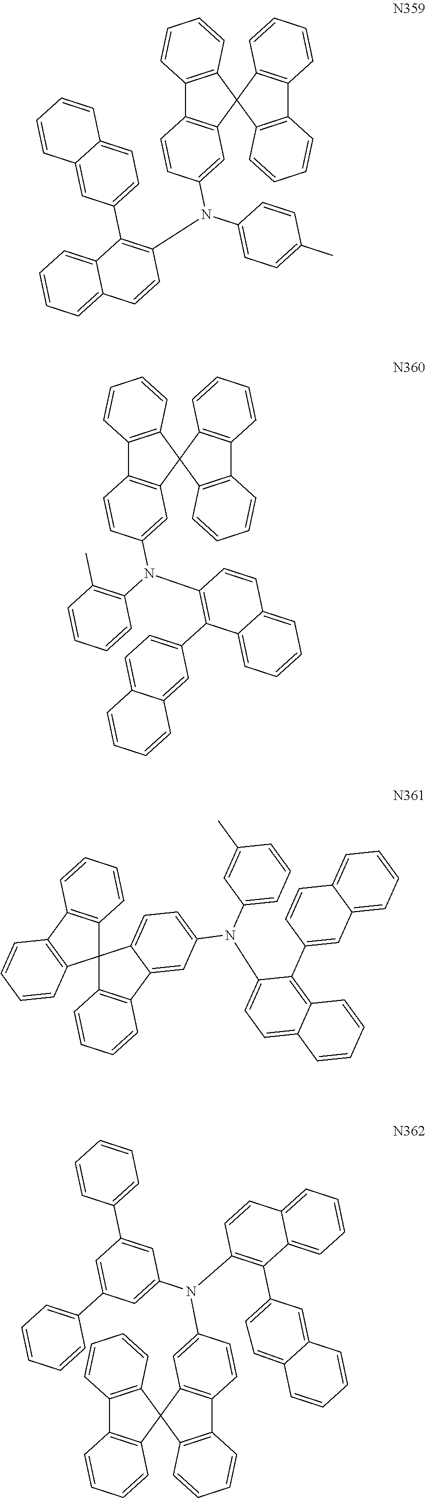 [SMILES: Cc1ccc(N(c2ccc3c(c2)C2(c4ccccc4-c4ccccc42)c2ccccc2-3)c2ccc3ccccc3c2-c2ccc3ccccc3c2)cc1 ; Cc1ccccc1N(c1ccc2c(c1)C1(c3ccccc3-c3ccccc31)c1ccccc1-2)c1ccc2ccccc2c1-c1ccc2ccccc2c1 ; Cc1cccc(N(c2ccc3c(c2)-c2ccccc2C32c3ccccc3-c3ccccc32)c2ccc3ccccc3c2-c2ccc3ccccc3c2)c1 ; c1ccc(-c2cc(-c3ccccc3)cc(N(c3ccc4c(c3)C3(c5ccccc5-c5ccccc53)c3ccccc3-4)c3ccc4ccccc4c3-c3ccc4ccccc4c3)c2)cc1]

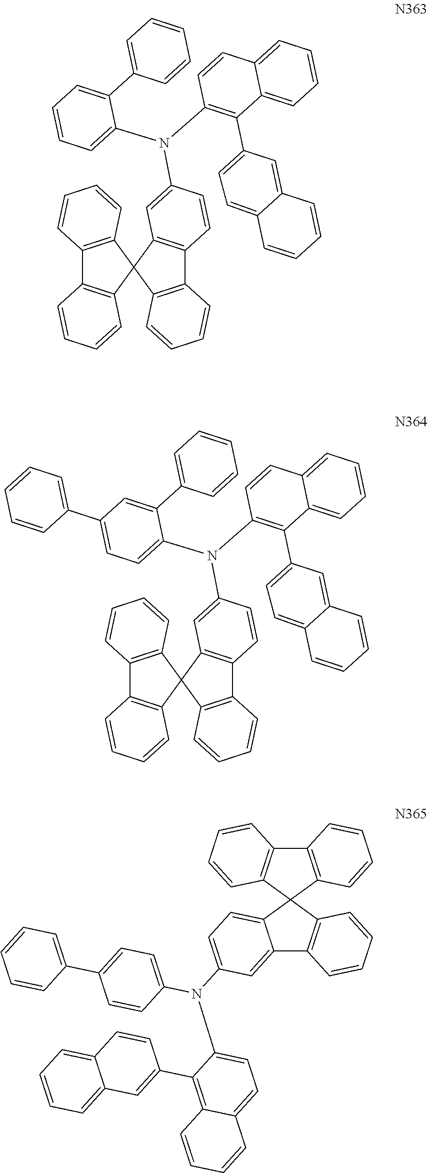 [SMILES: c1ccc(-c2ccccc2N(c2ccc3c(c2)C2(c4ccccc4-c4ccccc42)c2ccccc2-3)c2ccc3ccccc3c2-c2ccc3ccccc3c2)cc1 ; c1ccc(-c2ccc(N(c3ccc4c(c3)C3(c5ccccc5-c5ccccc53)c3ccccc3-4)c3ccc4ccccc4c3-c3ccc4ccccc4c3)c(-c3ccccc3)c2)cc1 ; c1ccc(-c2ccc(N(c3ccc4c(c3)-c3ccccc3C43c4ccccc4-c4ccccc43)c3ccc4ccccc4c3-c3ccc4ccccc4c3)cc2)cc1]

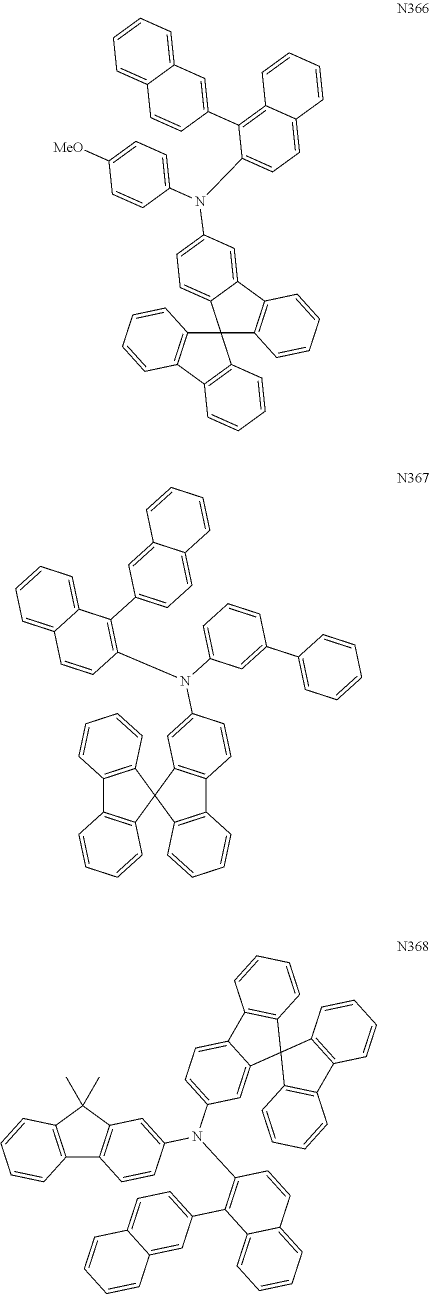 [SMILES: COc1ccc(N(c2ccc3c(c2)-c2ccccc2C32c3ccccc3-c3ccccc32)c2ccc3ccccc3c2-c2ccc3ccccc3c2)cc1 ; c1ccc(-c2cccc(N(c3ccc4c(c3)C3(c5ccccc5-c5ccccc53)c3ccccc3-4)c3ccc4ccccc4c3-c3ccc4ccccc4c3)c2)cc1 ; CC1(C)c2ccccc2-c2ccc(N(c3ccc4c(c3)C3(c5ccccc5-c5ccccc53)c3ccccc3-4)c3ccc4ccccc4c3-c3ccc4ccccc4c3)cc21]

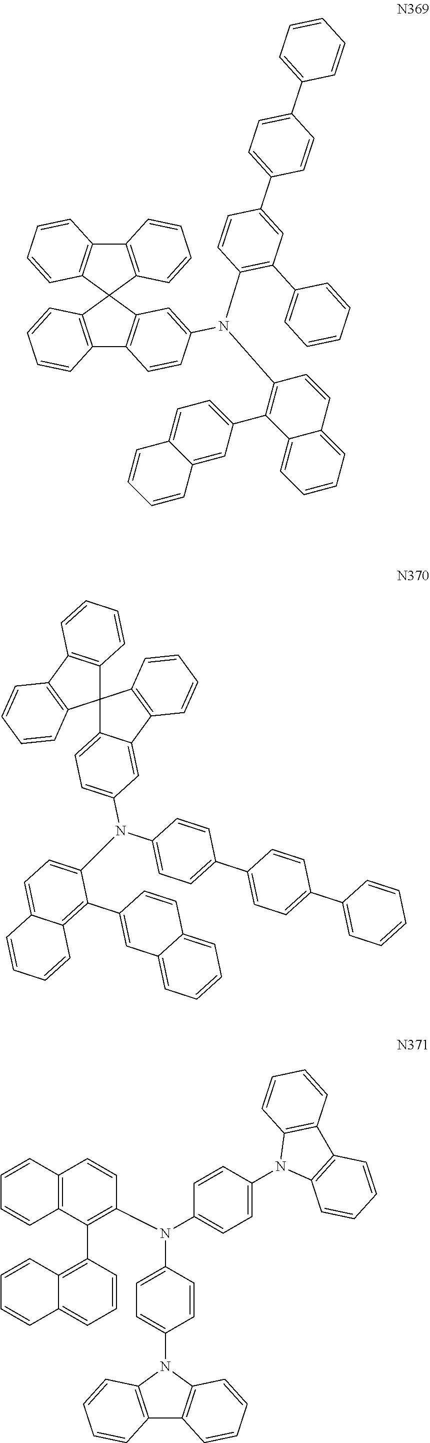 [SMILES: c1ccc(-c2ccc(-c3ccc(N(c4ccc5c(c4)C4(c6ccccc6-c6ccccc64)c4ccccc4-5)c4ccc5ccccc5c4-c4ccc5ccccc5c4)c(-c4ccccc4)c3)cc2)cc1 ; c1ccc(-c2ccc(-c3ccc(N(c4ccc5c(c4)-c4ccccc4C54c5ccccc5-c5ccccc54)c4ccc5ccccc5c4-c4ccc5ccccc5c4)cc3)cc2)cc1 ; c1ccc2c(-c3c(N(c4ccc(-n5c6ccccc6c6ccccc65)cc4)c4ccc(-n5c6ccccc6c6ccccc65)cc4)ccc4ccccc34)cccc2c1]

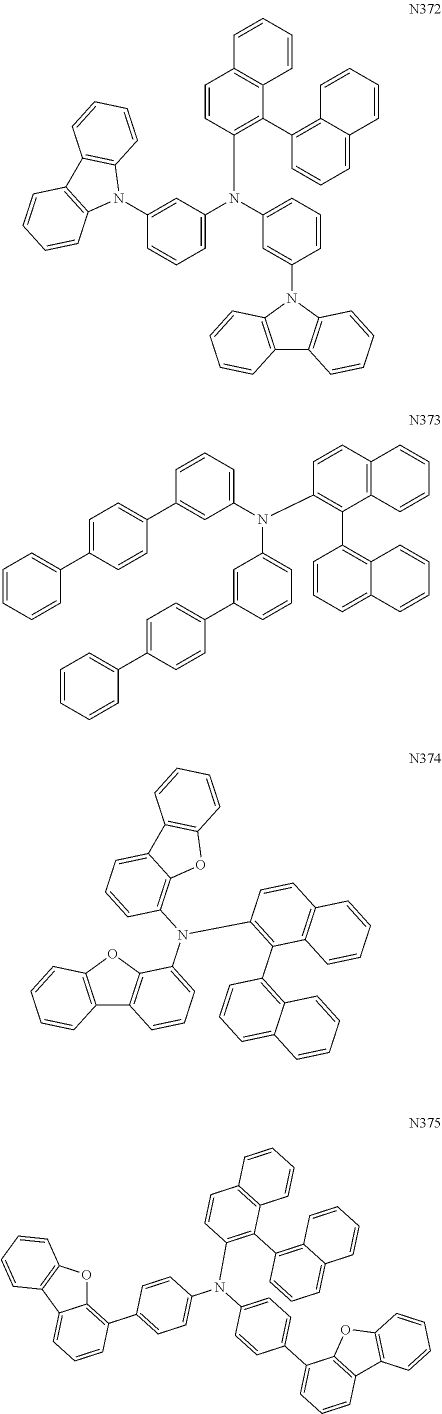 [SMILES: c1cc(N(c2cccc(-n3c4ccccc4c4ccccc43)c2)c2ccc3ccccc3c2-c2cccc3ccccc23)cc(-n2c3ccccc3c3ccccc32)c1 ; c1ccc(-c2ccc(-c3cccc(N(c4cccc(-c5ccc(-c6ccccc6)cc5)c4)c4ccc5ccccc5c4-c4cccc5ccccc45)c3)cc2)cc1 ; c1ccc2c(-c3c(N(c4cccc5c4oc4ccccc45)c4cccc5c4oc4ccccc45)ccc4ccccc34)cccc2c1 ; c1ccc2c(-c3c(N(c4ccc(-c5cccc6c5oc5ccccc56)cc4)c4ccc(-c5cccc6c5oc5ccccc56)cc4)ccc4ccccc34)cccc2c1]

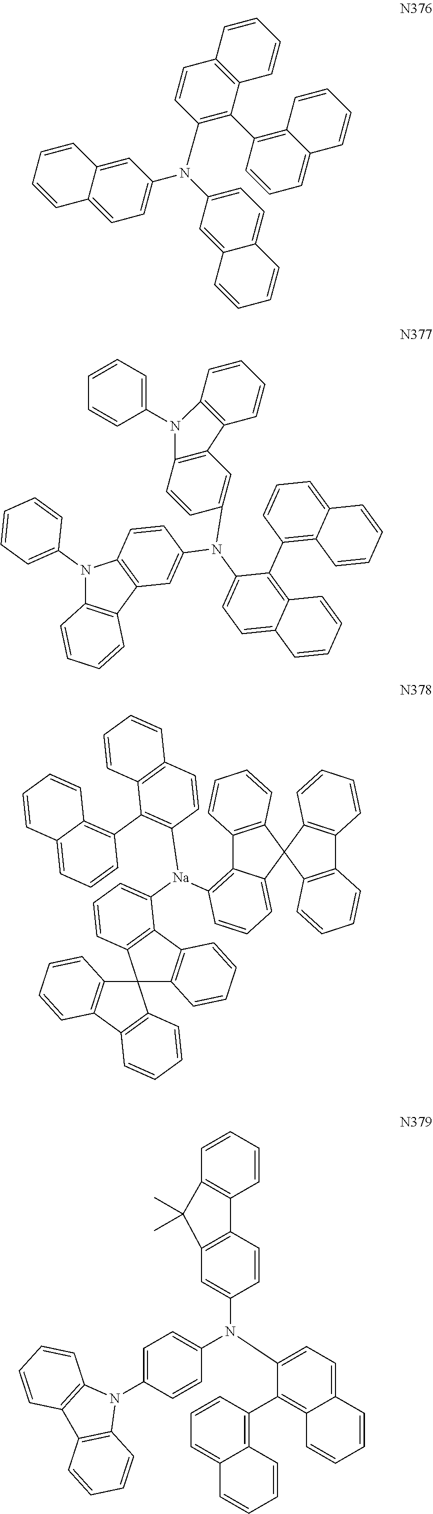

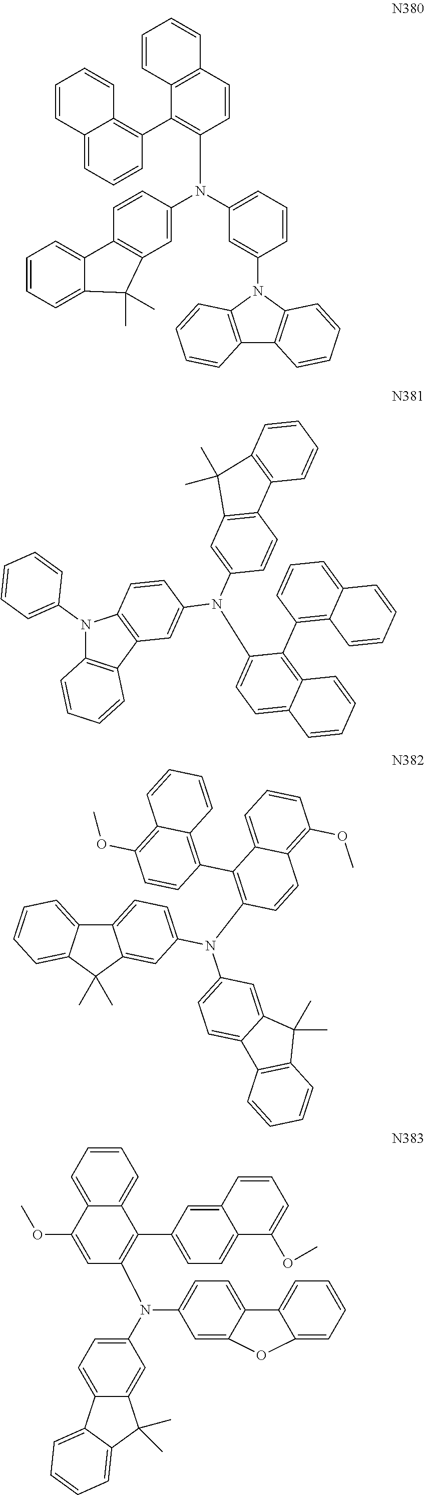

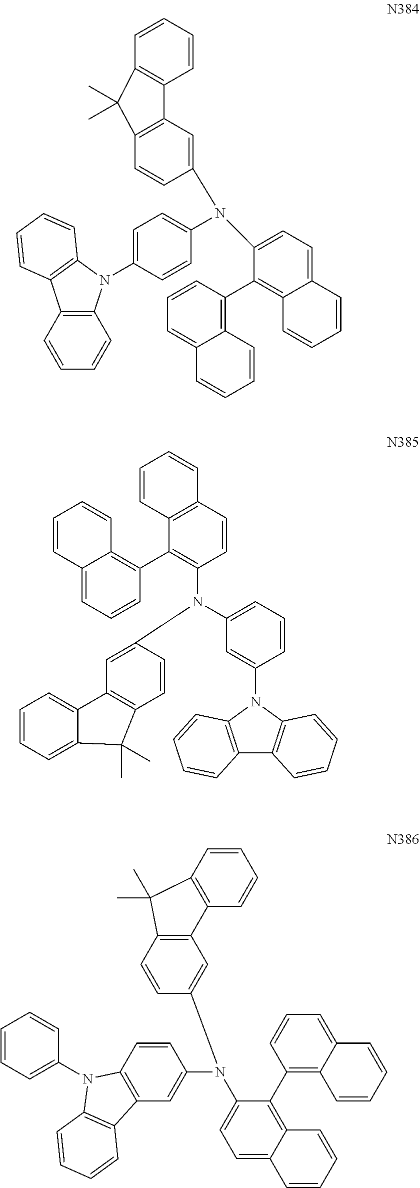 [SMILES: CC1(C)c2ccccc2-c2cc(N(c3ccc(-n4c5ccccc5c5ccccc54)cc3)c3ccc4ccccc4c3-c3cccc4ccccc34)ccc21 ; CC1(C)c2ccccc2-c2cc(N(c3cccc(-n4c5ccccc5c5ccccc54)c3)c3ccc4ccccc4c3-c3cccc4ccccc34)ccc21 ; CC1(C)c2ccccc2-c2cc(N(c3ccc4c(c3)c3ccccc3n4-c3ccccc3)c3ccc4ccccc4c3-c3cccc4ccccc34)ccc21]

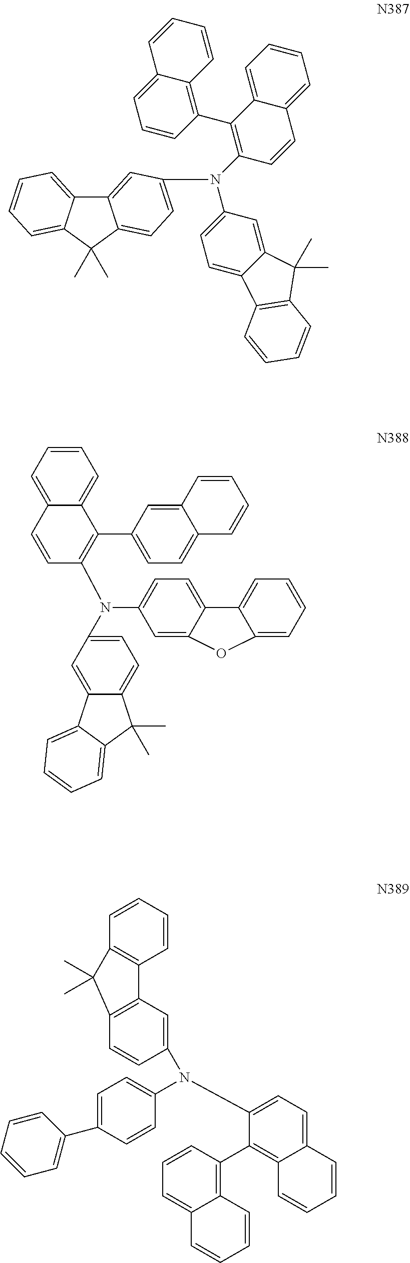 [SMILES: CC1(C)c2ccccc2-c2cc(N(c3ccc4c(c3)C(C)(C)c3ccccc3-4)c3ccc4ccccc4c3-c3cccc4ccccc34)ccc21 ; CC1(C)c2ccccc2-c2cc(N(c3ccc4c(c3)oc3ccccc34)c3ccc4ccccc4c3-c3ccc4ccccc4c3)ccc21 ; CC1(C)c2ccccc2-c2cc(N(c3ccc(-c4ccccc4)cc3)c3ccc4ccccc4c3-c3cccc4ccccc34)ccc21]

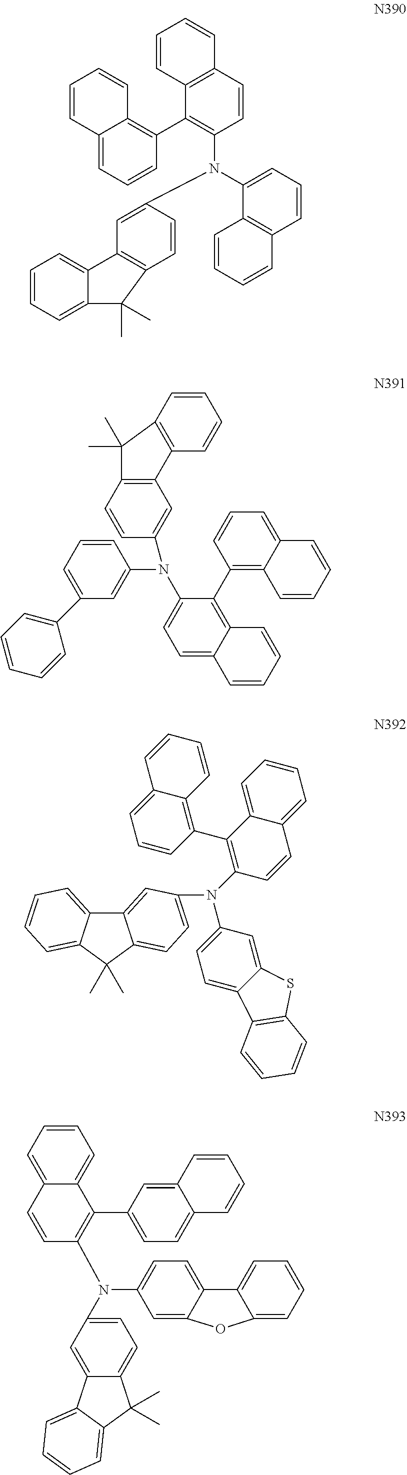 [SMILES: CC1(C)c2ccccc2-c2cc(N(c3ccc4ccccc4c3-c3cccc4ccccc34)c3cccc4ccccc34)ccc21 ; CC1(C)c2ccccc2-c2cc(N(c3cccc(-c4ccccc4)c3)c3ccc4ccccc4c3-c3cccc4ccccc34)ccc21 ; CC1(C)c2ccccc2-c2cc(N(c3ccc4c(c3)sc3ccccc34)c3ccc4ccccc4c3-c3cccc4ccccc34)ccc21 ; CC1(C)c2ccccc2-c2cc(N(c3ccc4c(c3)oc3ccccc34)c3ccc4ccccc4c3-c3ccc4ccccc4c3)ccc21]

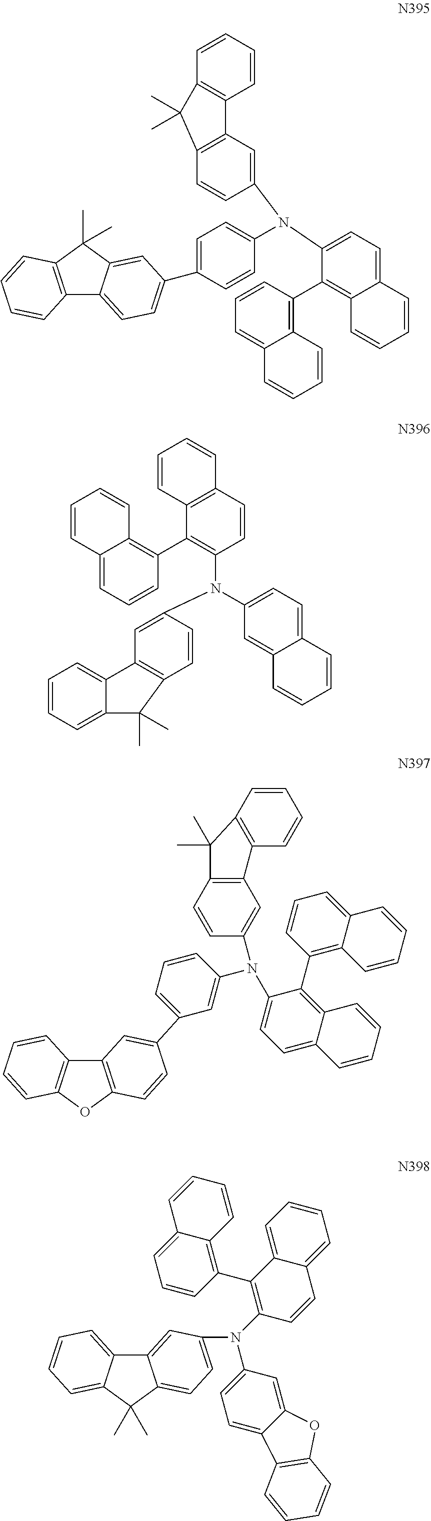 [SMILES: CC1(C)c2ccccc2-c2cc(N(c3ccc(-c4ccc5c(c4)C(C)(C)c4ccccc4-5)cc3)c3ccc4ccccc4c3-c3cccc4ccccc34)ccc21 ; CC1(C)c2ccccc2-c2cc(N(c3ccc4ccccc4c3)c3ccc4ccccc4c3-c3cccc4ccccc34)ccc21 ; CC1(C)c2ccccc2-c2cc(N(c3cccc(-c4ccc5oc6ccccc6c5c4)c3)c3ccc4ccccc4c3-c3cccc4ccccc34)ccc21 ; CC1(C)c2ccccc2-c2cc(N(c3ccc4c(c3)oc3ccccc34)c3ccc4ccccc4c3-c3cccc4ccccc34)ccc21]

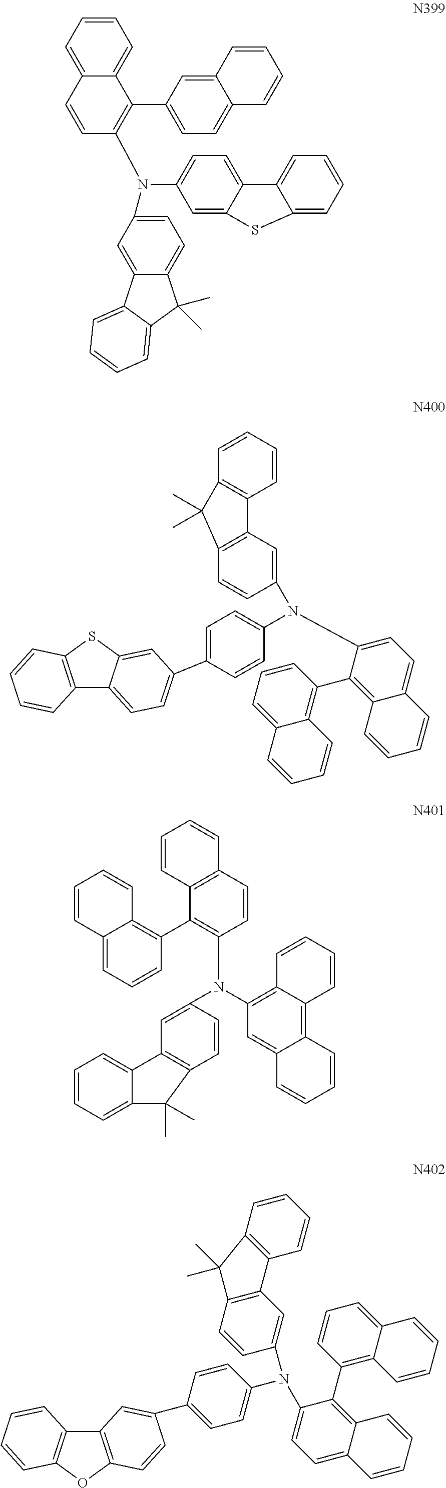 [SMILES: CC1(C)c2ccccc2-c2cc(N(c3ccc4c(c3)sc3ccccc34)c3ccc4ccccc4c3-c3ccc4ccccc4c3)ccc21 ; CC1(C)c2ccccc2-c2cc(N(c3ccc(-c4ccc5c(c4)sc4ccccc45)cc3)c3ccc4ccccc4c3-c3cccc4ccccc34)ccc21 ; CC1(C)c2ccccc2-c2cc(N(c3ccc4ccccc4c3-c3cccc4ccccc34)c3cc4ccccc4c4ccccc34)ccc21 ; CC1(C)c2ccccc2-c2cc(N(c3ccc(-c4ccc5oc6ccccc6c5c4)cc3)c3ccc4ccccc4c3-c3cccc4ccccc34)ccc21]

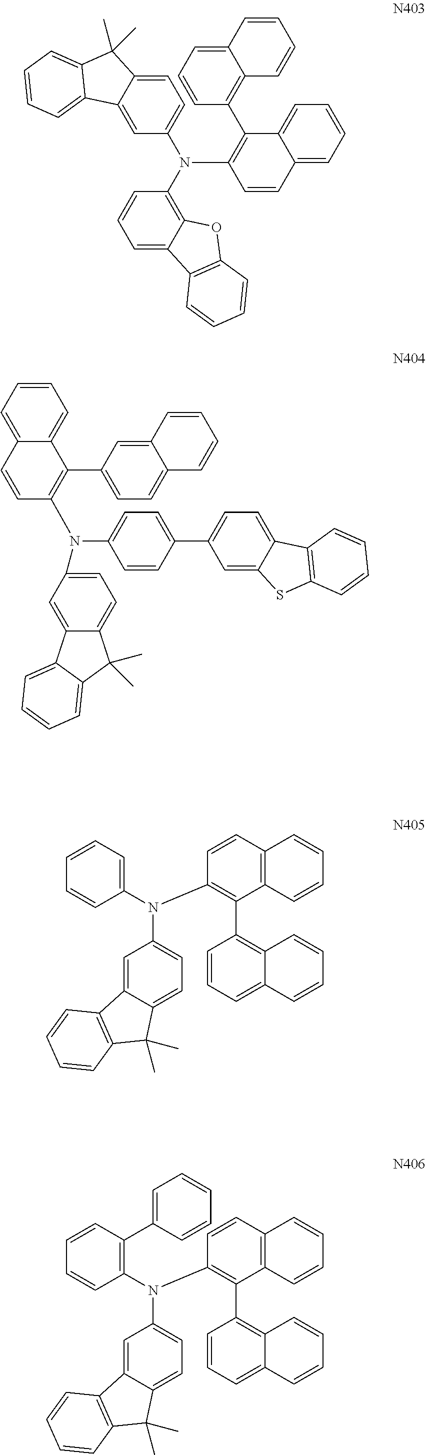 [SMILES: CC1(C)c2ccccc2-c2cc(N(c3ccc4ccccc4c3-c3cccc4ccccc34)c3cccc4c3oc3ccccc34)ccc21 ; CC1(C)c2ccccc2-c2cc(N(c3ccc(-c4ccc5c(c4)sc4ccccc45)cc3)c3ccc4ccccc4c3-c3ccc4ccccc4c3)ccc21 ; CC1(C)c2ccccc2-c2cc(N(c3ccccc3)c3ccc4ccccc4c3-c3cccc4ccccc34)ccc21 ; CC1(C)c2ccccc2-c2cc(N(c3ccccc3-c3ccccc3)c3ccc4ccccc4c3-c3cccc4ccccc34)ccc21]

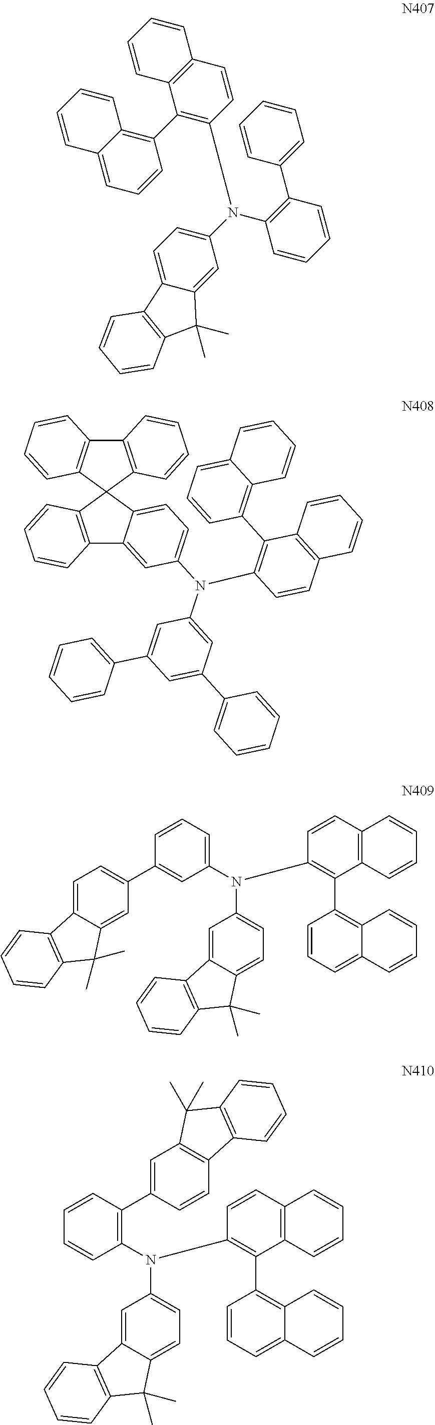

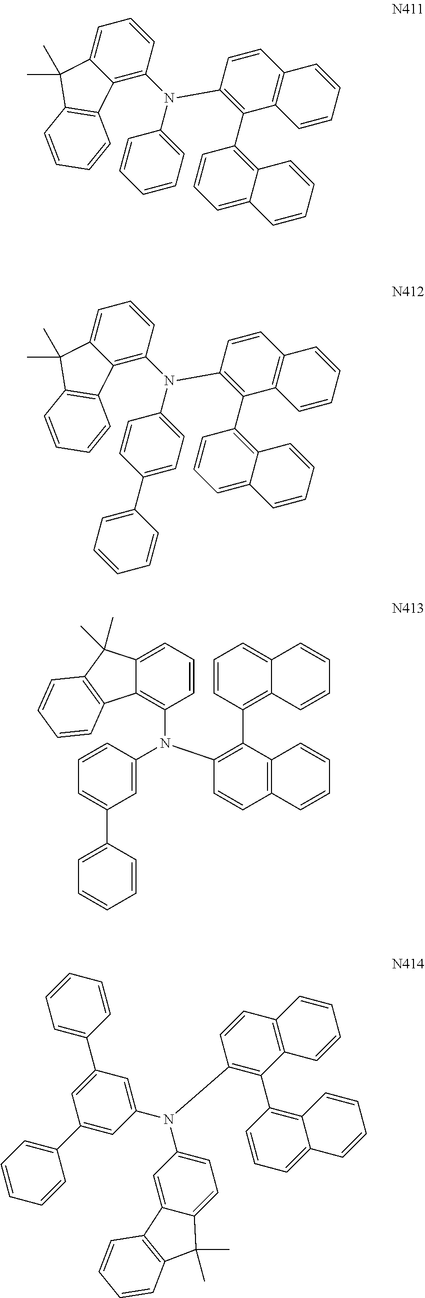

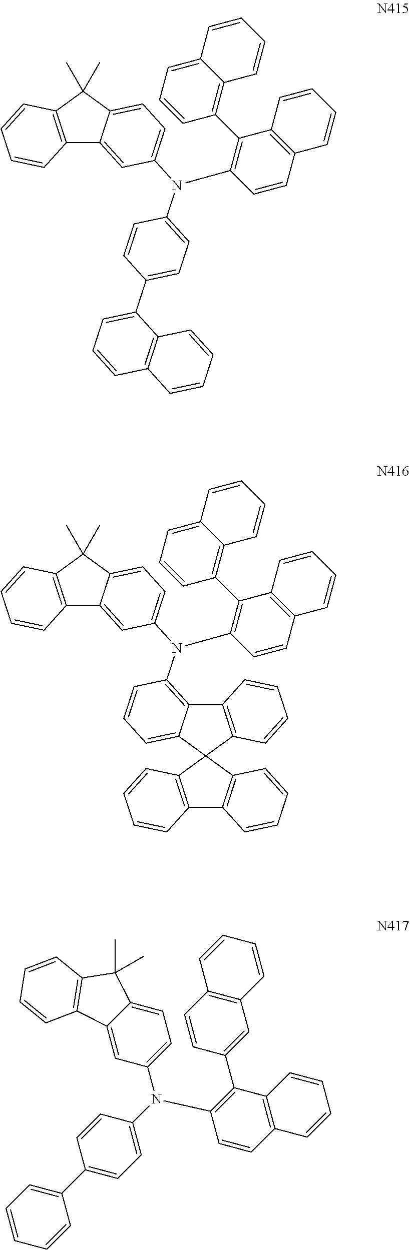 [SMILES: CC1(C)c2ccccc2-c2cc(N(c3ccc(-c4cccc5ccccc45)cc3)c3ccc4ccccc4c3-c3cccc4ccccc34)ccc21 ; CC1(C)c2ccccc2-c2cc(N(c3cccc4c3-c3ccccc3C43c4ccccc4-c4ccccc43)c3ccc4ccccc4c3-c3cccc4ccccc34)ccc21 ; CC1(C)c2ccccc2-c2cc(N(c3ccc(-c4ccccc4)cc3)c3ccc4ccccc4c3-c3ccc4ccccc4c3)ccc21]

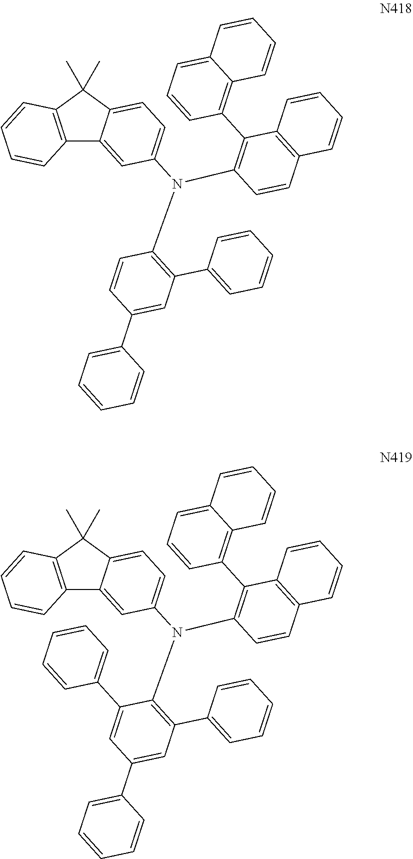 [SMILES: CC1(C)c2ccccc2-c2cc(N(c3ccc(-c4ccccc4)cc3-c3ccccc3)c3ccc4ccccc4c3-c3cccc4ccccc34)ccc21 ; CC1(C)c2ccccc2-c2cc(N(c3ccc4ccccc4c3-c3cccc4ccccc34)c3c(-c4ccccc4)cc(-c4ccccc4)cc3-c3ccccc3)ccc21]

The present invention provides an organic electroluminescent device, including a first electrode, a second electrode, and at least one organic layer inserted between the first electrode and the second organic compounds, where the organic layer includes the above compound.

In the above organic electroluminescent device, preferably, the organic layer includes a hole transport region, and the hole transport region contains the above compound, more preferably, the hole transport region includes a hole transport layer and/or an electron blocking layer, where at least one of the hole transport layer and the electron blocking layer contains the above compound.

The present invention provides an application of the above compound as a hole transport layer and/or an electron blocking layer in the organic electroluminescent device; but the organic layer of the compound of the present invention is not limited to be used in the hole transport layer and the electron blocking layer. Moreover, the compound of the present invention may be applied in an organic electron device. The organic electron device may be cited below, for example, an organic electroluminescent device, a lighting element, an organic thin film transistor, an organic field effect transistor, an organic thin film solar cell, an information label, an electronic artificial skin sheet, a sheet-type scanner, electronic paper or an organic EL panel.

### Preferred Embodiment III

The compound of the present invention preferably has a structure as shown in Formula (III):

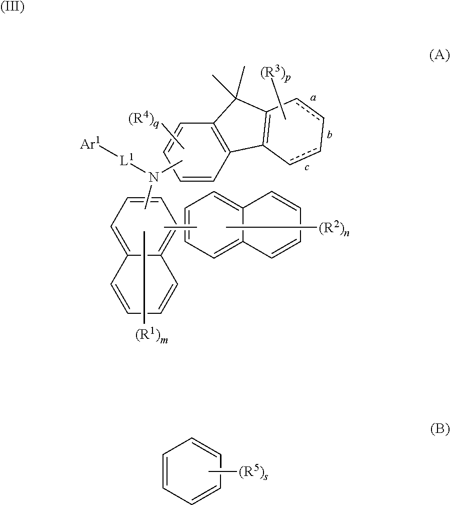

Formula (B) is fused to Formula (A) in the dotted line along any one of the dotted line of a, b or c;

L¹ is selected from one of a single bond, substituted or unsubstituted C₁-C₁₀ alkylene, substituted or unsubstituted C₆-C₃₀ arylene, a substituted or unsubstituted C₃-C₃₀ heteroarylene group;

Ar¹ is selected from one of substituted or unsubstituted C₆-C₃₀ aryl or substituted or unsubstituted C₃-C₃₀ heteroaryl;

R¹, R², R³, R⁴ and R⁵ are each independently selected from a halogen, amino, cyano, nitryl, an ester group, hydroxyl, a C₁-C₁₀ silicyl, a substituted or unsubstituted C₁-C₁₀ chain-typed alkyl, a substituted or unsubstituted C₁-C₁₀ cycloalkyl, a substituted or unsubstituted C₂-C₁₀ alkenyl, a substituted or unsubstituted C₂-C₁₀ alkynyl, a substituted or unsubstituted C₁-C₁₀ chain-typed alkoxy, a substituted or unsubstituted C₃-C₁₀ cycloalkoxy, a substituted or unsubstituted C₆-C₃₀ arylamino, a substituted or unsubstituted C₃-C₃₀ heteroarylamino, a substituted or unsubstituted C₆-C₃₀ aryl, a substituted or unsubstituted C₃-C₃₀ heteroaryl;

m is an integer of 0-6, for example, 1, 2, 3, 4, 5, and the like, and when m≥2, R¹ is same or different;

n is an integer of 0-7, for example, 1, 2, 3, 4, 5, 6, and the like, and when m≥2, R² is same or different;

p is an integer of 0-2, for example, 1, 2, 3, 4, 5, and the like, and when p=2, R³ is same or different;

q is an integer of 0-3, for example, 1, 2, 3, and the like, and when q≥2, R⁴ is same or different;

s is an integer of 0-4, and when s≥2, R⁵ is same or different;

when the above groups have a substituent, the substituent is selected from one or a combination of at least two of a halogen, cyano, a C₁-C₁₀ chain-typed alkyl, a C₃-C₁₀ cycloalkyl, a C₁-C₆ alkoxy, a C₁-C₆ thioalkoxy, a C₆-C₃₀ arylamino, a C₃-C₃₀ heteroarylamino, a C₆-C₃₀ monocyclic aryl, a C₁₀-C₃₀ fused-cyclic aryl, a C₃-C₃₀ monocyclic heteroaryl, and a C₆-C₃₀ fused-cyclic heteroaryl.

The present invention provides a novel compound. The compound contains a structure that two units of dinaphthalene and benzofluorene are respectively linked with N atom, and is further matched with Ar¹, such that the compound has good hole injection and hole transport performances, good refraction coefficient, higher phase-transition temperature. Therefore, the OLED device containing the compound is featured by high luminous efficiency, low driving voltage and long service life.

The above compound of the present invention has three fused sites, a, b and c; and may be divided into three structures as shown in the following Formula (III-1), Formula (III-2), and Formula (III-3) according to different fused positions.

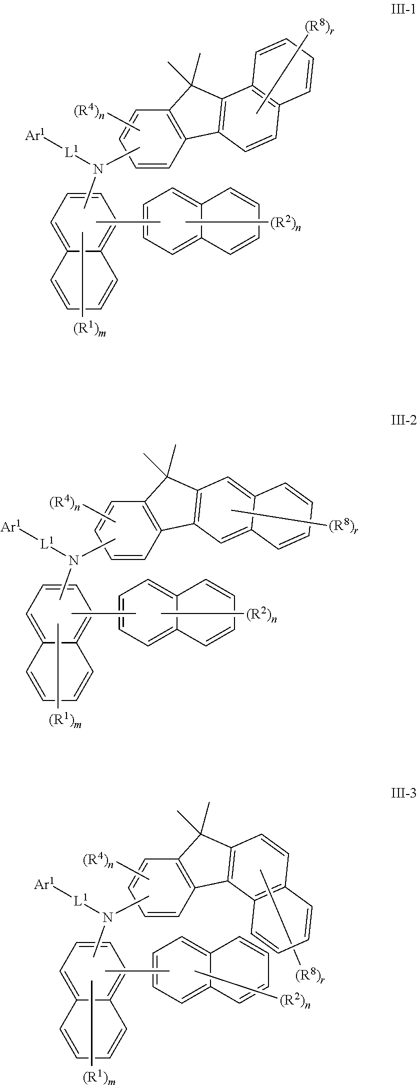

The R⁶ has a selection range the same as that of R¹-R⁵; the r is an integer of 0-6, and when r≥2, R⁶ is same or different.

The above compound of the present invention preferably has the structure as shown in Formula (III-2); that is, fluorenyl and benzene ring are preferably fused in the position as shown in Formula (III-2). This is because the molecular conformation fused at 6, 7 positions has superior arrangement, which not only effectively reduces the energy barrier of hole injection, but also improves hole transport capacity, thereby further improving device performance.

The above compound of the present invention also preferably has a structure as shown in Formula (3-1):

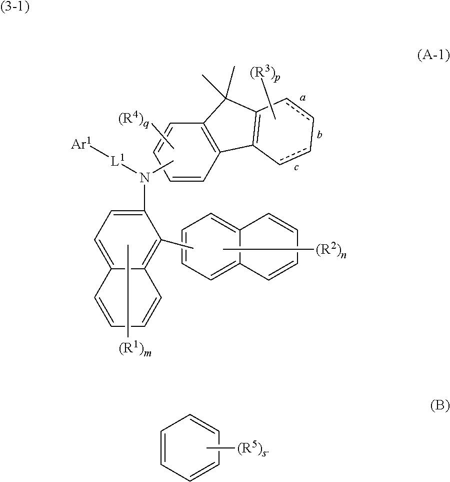

Formula (B) is fused to Formula (A) along any one of the dotted line of a, b or c;

The L¹, Ar¹, R¹, R², R, R⁴, R⁵, s, p, n, m and q have the same selection range as the preceding description.

In the present invention, naphthyl and arylamido are preferably substituted in an ortho position. Such a specific structure may not only effectively reduce the energy barrier of hole injection, but also may improve the hole transport capacity, thereby further improving the luminous efficiency of the device, reducing driving voltage and prolonging the service life.

The Formula (3-1) of the present invention has three fused sites, a, b and c; and may be specifically divided into three structures as shown in the following Formula (3-1-1), Formula (3-1-2), and Formula (3-1-3) according to different fused positions.

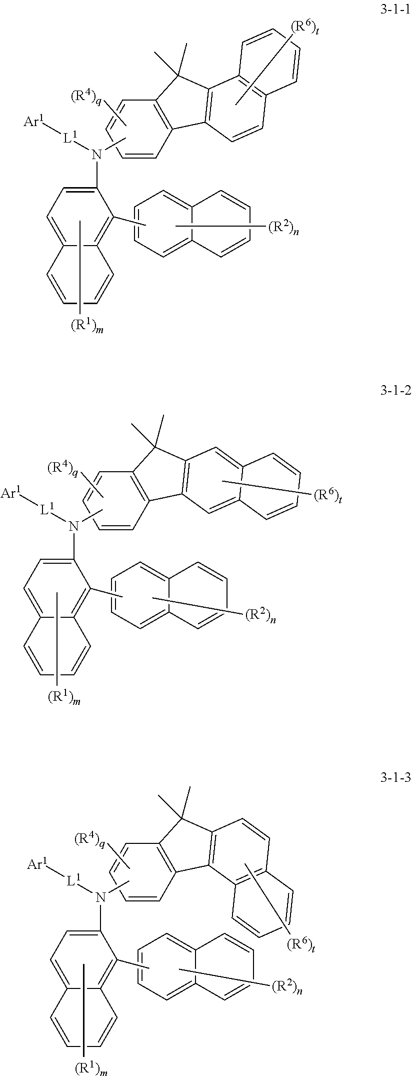

The L¹, Ar¹, R¹, R², R⁶, R⁴, m, n, r and q have the same selection range as the preceding description.

In the above Formula (3-1), the Formula (A-1) and the Formula (B) are preferably fused in the b position, namely, the structure as shown in Formula (3-1-2) is preferred.

The above compound of the present invention also preferably has a structure as shown in the following Formula (3-2):

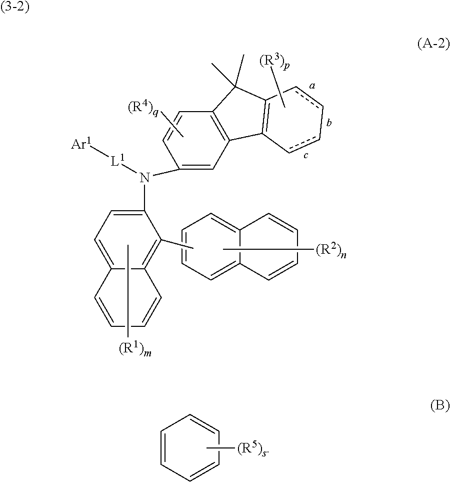

Formula (B) is fused to Formula (A) along any one of the dotted line of a, b or c;

the L¹, Ar¹, R¹, R², R³, R⁴, R⁵, s, p, n, m and q have the same selection range as the preceding description.

The Formula (3-2) of the present invention has three fused sites, a, b and c; and may be specifically divided into three structures as shown in the following Formula (3-2-1), Formula (3-2-2), and Formula (3-2-3) according to different fused positions:

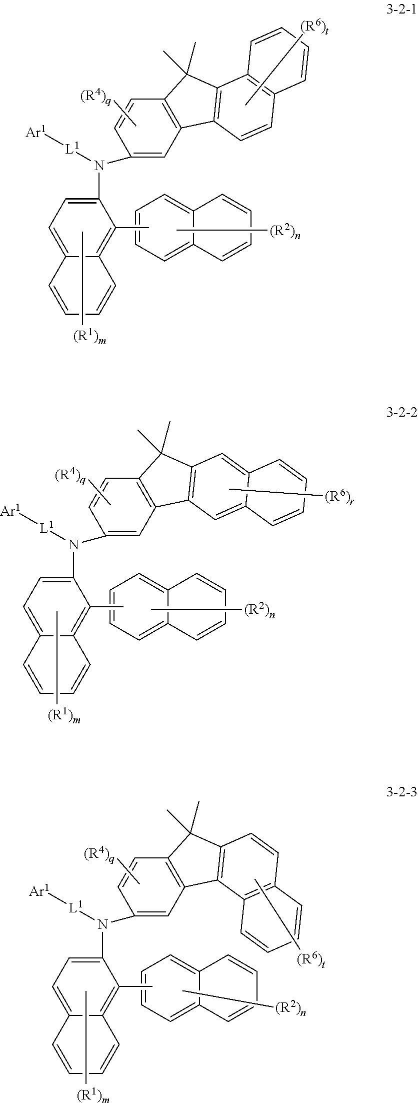

the L¹, Ar¹, R¹, R², R⁶, R⁴, m, n, r and q have the same selection range as the preceding description.

In the above Formula (3-2), the Formula (A-2) and the Formula (B) are further preferably fused in the b position, namely, the structure as shown in Formula (3-2-2) is preferred.

In the above Formulas (III), (3-1), and (3-2), s, p, n, m and q are preferably 0. In the above Formulas (III-1), (III-2), (III-3), (3-1-1), (3-1-2), (3-1-3), (3-2-1), (3-2-2), and (3-2-3), n, m, q and r are preferably 0.

The above compound of the present invention preferably has the structures in Formulas (3-2-1), (3-2-2), and (3-2-3), where n, m, q and r are 0, more preferably, has the structure in Formula (3-2-2), where n, m, q and r are 0.

In the above compound of the present invention, L¹ is preferably selected from a single bond or substituted or unsubstituted phenylene, more preferably, a single bond; when the above group has a substituent, the substituent is selected from one or a combination of at least two of a halogen, cyano, a C₁-C₁₀ chain-typed alkyl, a C₃-C₁₀ cycloalkyl, a C₁-C₆ alkoxy, a C₁-C₆ thioalkoxy, a C₆-C₃₀ arylamino, a C₃-C₃₀ heteroarylamino, a C₆-C₃₀ monocyclic aryl, a C₁₀-C₃₀ fused-cyclic aryl, a C₃-C₃₀ monocyclic heteroaryl, and a C₆-C₃₀ fused-cyclic heteroaryl.

In the above compound of the present invention, Ar¹ is preferably selected from substituted or unsubstituted phenyl, substituted or unsubstituted naphthyl, substituted or unsubstituted phenanthryl, substituted or unsubstituted dibenzofuryl, substituted or unsubstituted dibenzothienyl, substituted or unsubstituted carbazolyl; when the above group has a substituent, the substituent is selected from one or a combination of at least two of a halogen, cyano, a C₁-C₁₀ chain-typed alkyl, a C₃-C₁₀ cycloalkyl, a C₁-C₆ alkoxy, a C₁-C₆ thioalkoxy, a C₆-C₃₀ arylamino, a C₃-C₃₀ heteroarylamino, a C₆-C₃₀ monocyclic aryl, a C₁₀-C₃₀ fused-cyclic aryl, a C₃-C₃₀ monocyclic heteroaryl, and a C₆-C₃₀ fused-cyclic heteroaryl.

In the above compound of the present invention, the -L-Ar¹ is preferably selected from one of phenyl, biphenylyl, terphenylyl, dibenzofuran, dibenzothiophene, carbazolyl or phenanthryl;

The compound having the structure as shown in the above Formula (III) of the present invention is preferably any one of the following compounds T1-T255, but these compounds are merely representative.

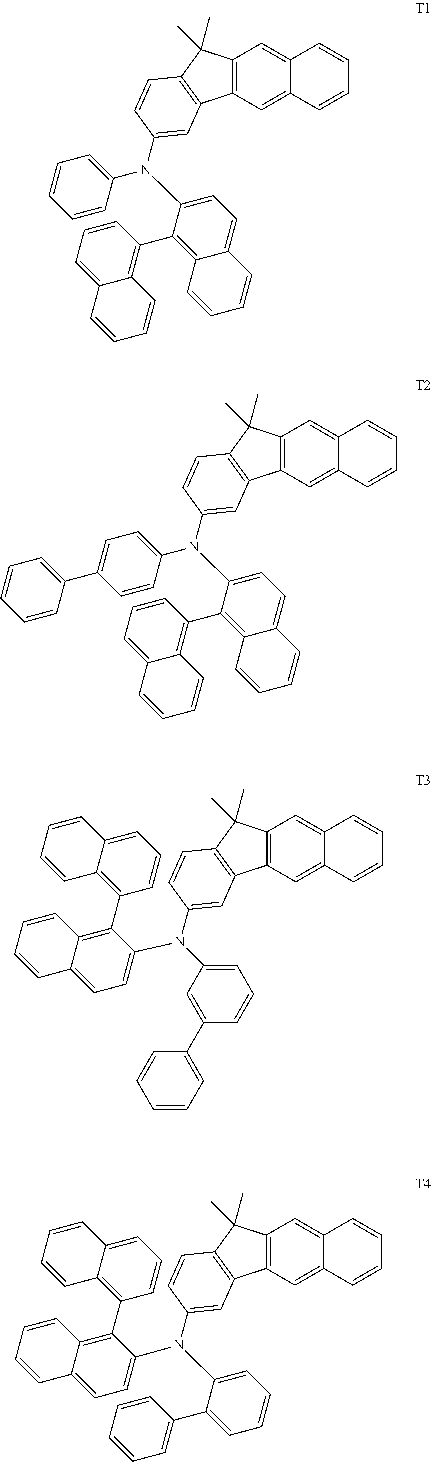 [SMILES: CC1(C)c2ccc(N(c3ccccc3)c3ccc4ccccc4c3-c3cccc4ccccc34)cc2-c2cc3ccccc3cc21 ; CC1(C)c2ccc(N(c3ccc(-c4ccccc4)cc3)c3ccc4ccccc4c3-c3cccc4ccccc34)cc2-c2cc3ccccc3cc21 ; CC1(C)c2ccc(N(c3cccc(-c4ccccc4)c3)c3ccc4ccccc4c3-c3cccc4ccccc34)cc2-c2cc3ccccc3cc21 ; CC1(C)c2ccc(N(c3ccccc3-c3ccccc3)c3ccc4ccccc4c3-c3cccc4ccccc34)cc2-c2cc3ccccc3cc21]

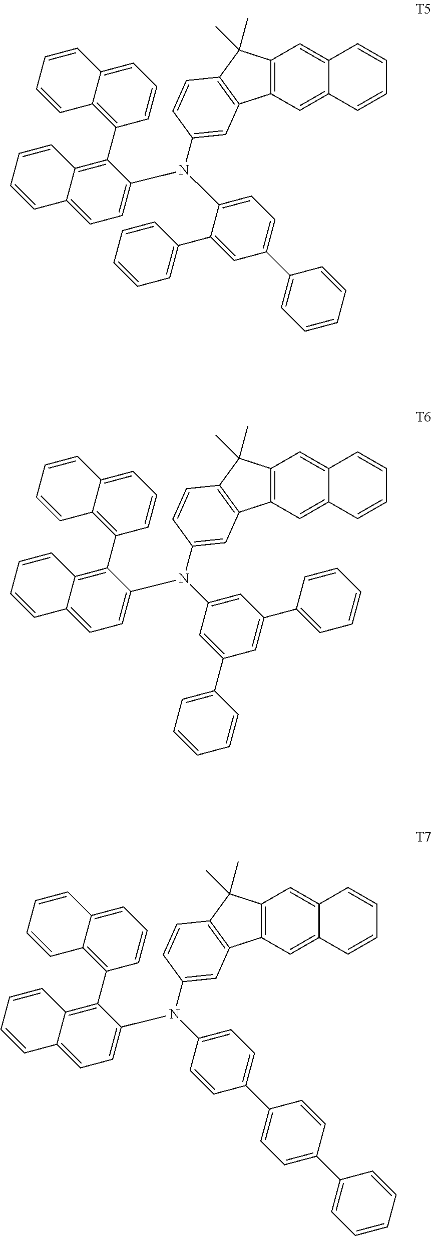 [SMILES: CC1(C)c2ccc(N(c3ccc(-c4ccccc4)cc3-c3ccccc3)c3ccc4ccccc4c3-c3cccc4ccccc34)cc2-c2cc3ccccc3cc21 ; CC1(C)c2ccc(N(c3cc(-c4ccccc4)cc(-c4ccccc4)c3)c3ccc4ccccc4c3-c3cccc4ccccc34)cc2-c2cc3ccccc3cc21 ; CC1(C)c2ccc(N(c3ccc(-c4ccc(-c5ccccc5)cc4)cc3)c3ccc4ccccc4c3-c3cccc4ccccc34)cc2-c2cc3ccccc3cc21]

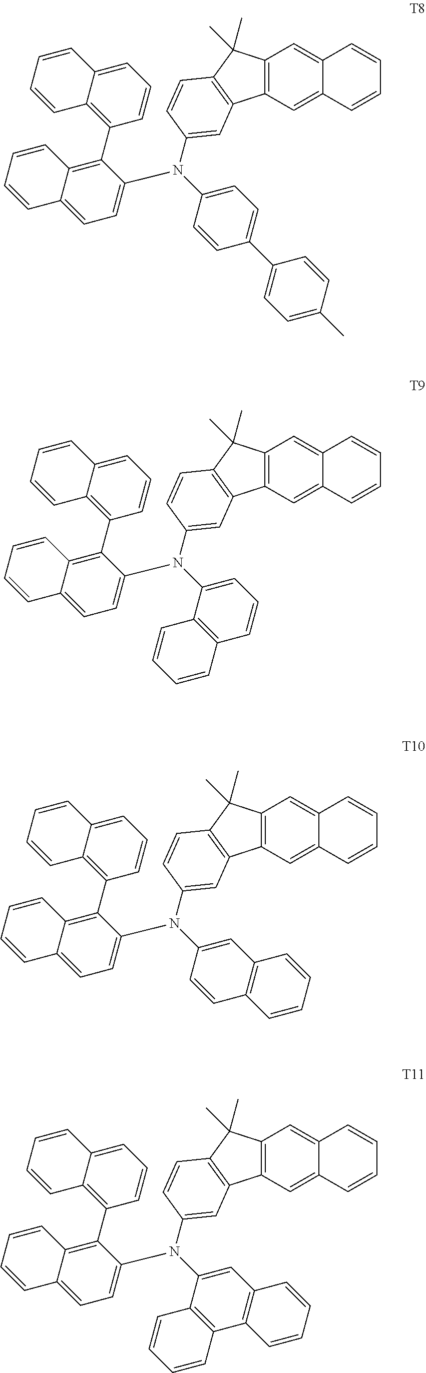 [SMILES: Cc1ccc(-c2ccc(N(c3ccc4c(c3)-c3cc5ccccc5cc3C4(C)C)c3ccc4ccccc4c3-c3cccc4ccccc34)cc2)cc1 ; CC1(C)c2ccc(N(c3ccc4ccccc4c3-c3cccc4ccccc34)c3cccc4ccccc34)cc2-c2cc3ccccc3cc21 ; CC1(C)c2ccc(N(c3ccc4ccccc4c3)c3ccc4ccccc4c3-c3cccc4ccccc34)cc2-c2cc3ccccc3cc21 ; CC1(C)c2ccc(N(c3ccc4ccccc4c3-c3cccc4ccccc34)c3cc4ccccc4c4ccccc34)cc2-c2cc3ccccc3cc21]

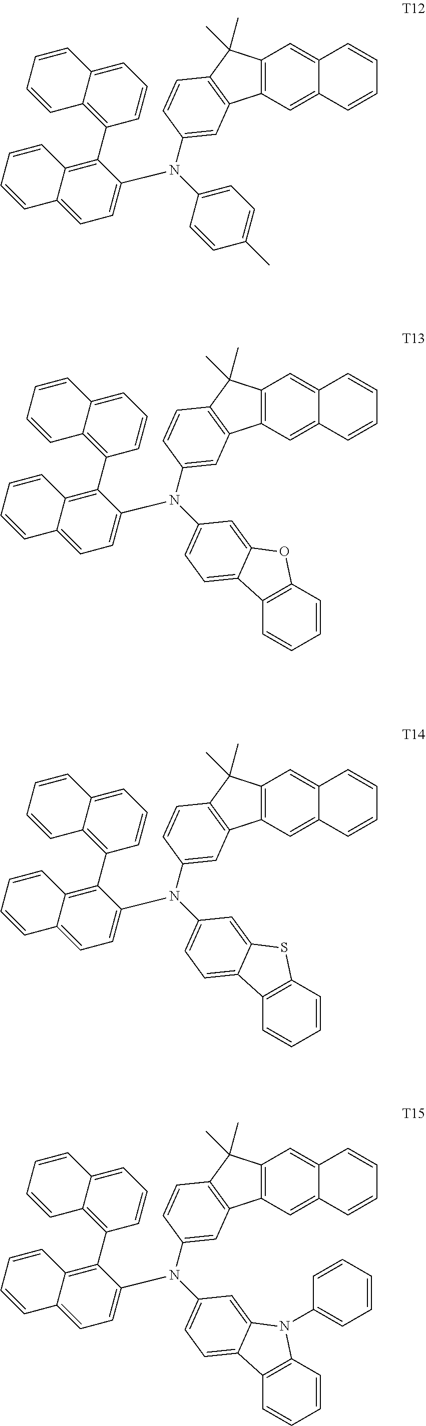 [SMILES: Cc1ccc(N(c2ccc3c(c2)-c2cc4ccccc4cc2C3(C)C)c2ccc3ccccc3c2-c2cccc3ccccc23)cc1 ; CC1(C)c2ccc(N(c3ccc4c(c3)oc3ccccc34)c3ccc4ccccc4c3-c3cccc4ccccc34)cc2-c2cc3ccccc3cc21 ; CC1(C)c2ccc(N(c3ccc4c(c3)sc3ccccc34)c3ccc4ccccc4c3-c3cccc4ccccc34)cc2-c2cc3ccccc3cc21 ; CC1(C)c2ccc(N(c3ccc4c5ccccc5n(-c5ccccc5)c4c3)c3ccc4ccccc4c3-c3cccc4ccccc34)cc2-c2cc3ccccc3cc21]

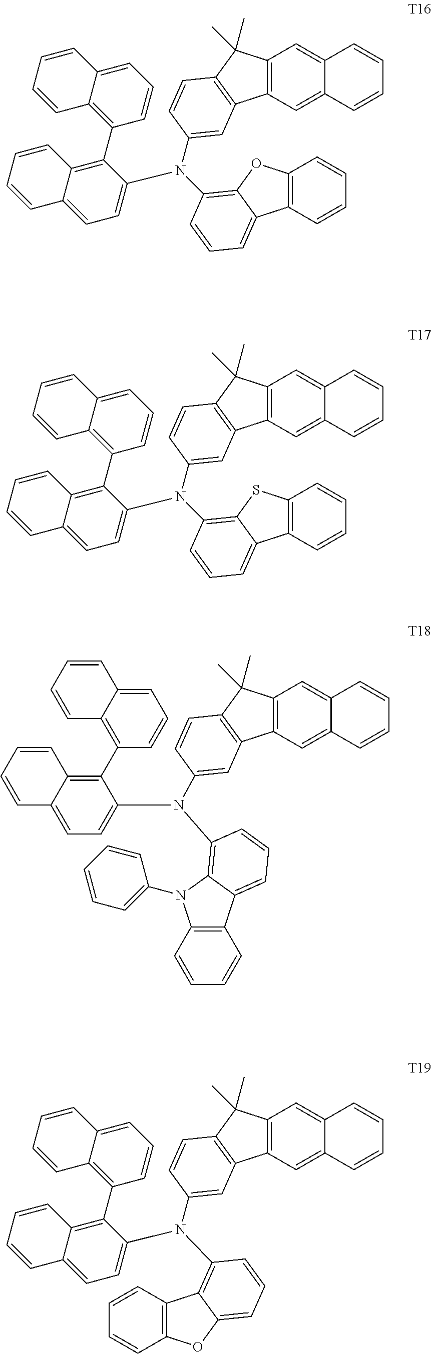 [SMILES: CC1(C)c2ccc(N(c3ccc4ccccc4c3-c3cccc4ccccc34)c3cccc4c3oc3ccccc34)cc2-c2cc3ccccc3cc21 ; CC1(C)c2ccc(N(c3ccc4ccccc4c3-c3cccc4ccccc34)c3cccc4c3sc3ccccc34)cc2-c2cc3ccccc3cc21 ; CC1(C)c2ccc(N(c3ccc4ccccc4c3-c3cccc4ccccc34)c3cccc4c5ccccc5n(-c5ccccc5)c34)cc2-c2cc3ccccc3cc21 ; CC1(C)c2ccc(N(c3ccc4ccccc4c3-c3cccc4ccccc34)c3cccc4oc5ccccc5c34)cc2-c2cc3ccccc3cc21]

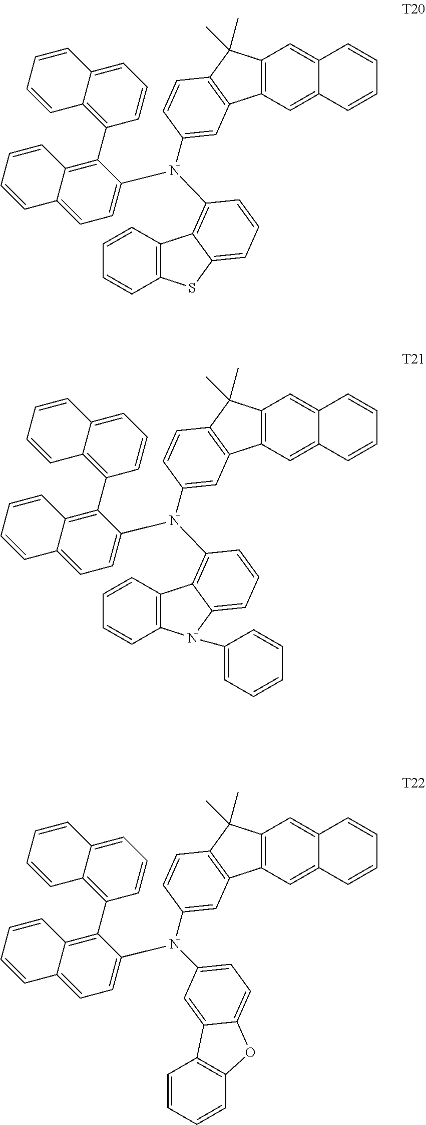 [SMILES: CC1(C)c2ccc(N(c3ccc4ccccc4c3-c3cccc4ccccc34)c3cccc4sc5ccccc5c34)cc2-c2cc3ccccc3cc21 ; CC1(C)c2ccc(N(c3ccc4ccccc4c3-c3cccc4ccccc34)c3cccc4c3c3ccccc3n4-c3ccccc3)cc2-c2cc3ccccc3cc21 ; CC1(C)c2ccc(N(c3ccc4oc5ccccc5c4c3)c3ccc4ccccc4c3-c3cccc4ccccc34)cc2-c2cc3ccccc3cc21]

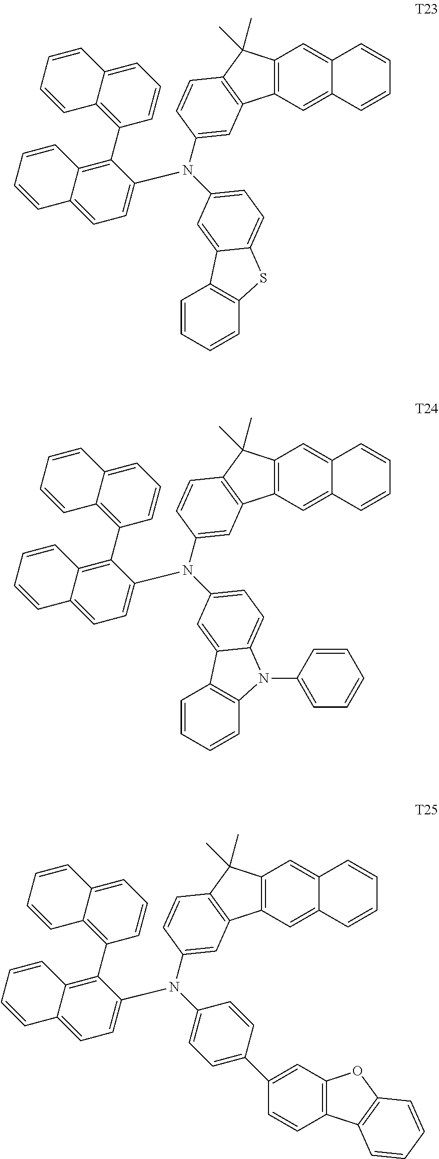 [SMILES: CC1(C)c2ccc(N(c3ccc4sc5ccccc5c4c3)c3ccc4ccccc4c3-c3cccc4ccccc34)cc2-c2cc3ccccc3cc21 ; CC1(C)c2ccc(N(c3ccc4c(c3)c3ccccc3n4-c3ccccc3)c3ccc4ccccc4c3-c3cccc4ccccc34)cc2-c2cc3ccccc3cc21 ; CC1(C)c2ccc(N(c3ccc(-c4ccc5c(c4)oc4ccccc45)cc3)c3ccc4ccccc4c3-c3cccc4ccccc34)cc2-c2cc3ccccc3cc21]

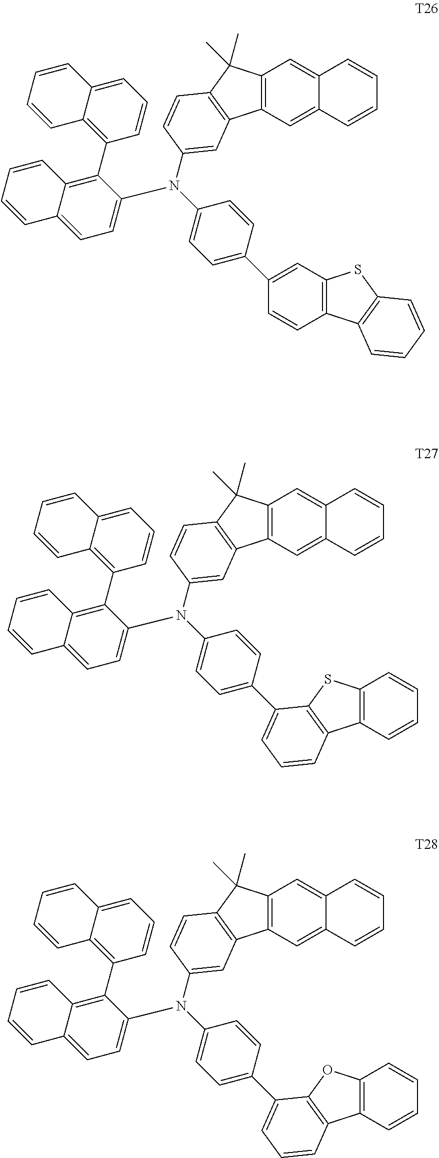 [SMILES: CC1(C)c2ccc(N(c3ccc(-c4ccc5c(c4)sc4ccccc45)cc3)c3ccc4ccccc4c3-c3cccc4ccccc34)cc2-c2cc3ccccc3cc21 ; CC1(C)c2ccc(N(c3ccc(-c4cccc5c4sc4ccccc45)cc3)c3ccc4ccccc4c3-c3cccc4ccccc34)cc2-c2cc3ccccc3cc21 ; CC1(C)c2ccc(N(c3ccc(-c4cccc5c4oc4ccccc45)cc3)c3ccc4ccccc4c3-c3cccc4ccccc34)cc2-c2cc3ccccc3cc21]

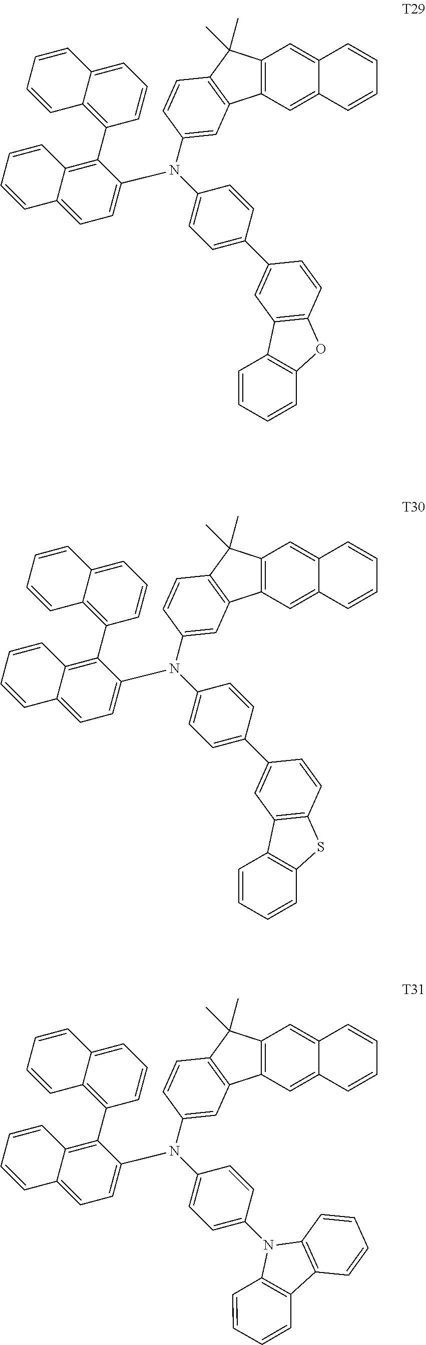 [SMILES: CC1(C)c2ccc(N(c3ccc(-c4ccc5oc6ccccc6c5c4)cc3)c3ccc4ccccc4c3-c3cccc4ccccc34)cc2-c2cc3ccccc3cc21 ; CC1(C)c2ccc(N(c3ccc(-c4ccc5sc6ccccc6c5c4)cc3)c3ccc4ccccc4c3-c3cccc4ccccc34)cc2-c2cc3ccccc3cc21 ; CC1(C)c2ccc(N(c3ccc(-n4c5ccccc5c5ccccc54)cc3)c3ccc4ccccc4c3-c3cccc4ccccc34)cc2-c2cc3ccccc3cc21]

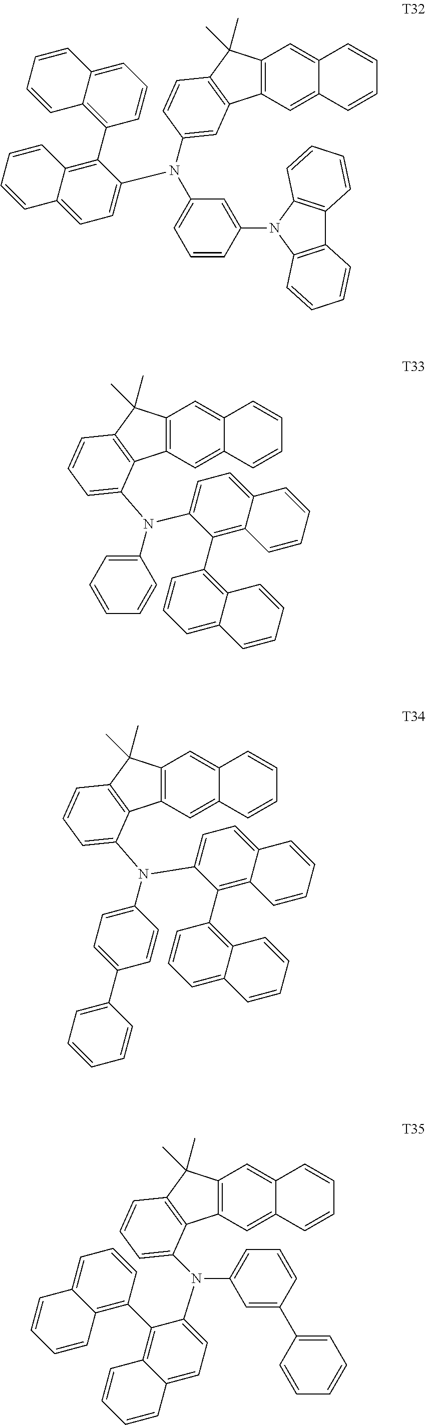 [SMILES: CC1(C)c2ccc(N(c3cccc(-n4c5ccccc5c5ccccc54)c3)c3ccc4ccccc4c3-c3cccc4ccccc34)cc2-c2cc3ccccc3cc21 ; CC1(C)c2cc3ccccc3cc2-c2c(N(c3ccccc3)c3ccc4ccccc4c3-c3cccc4ccccc34)cccc21 ; CC1(C)c2cc3ccccc3cc2-c2c(N(c3ccc(-c4ccccc4)cc3)c3ccc4ccccc4c3-c3cccc4ccccc34)cccc21 ; CC1(C)c2cc3ccccc3cc2-c2c(N(c3cccc(-c4ccccc4)c3)c3ccc4ccccc4c3-c3cccc4ccccc34)cccc21]

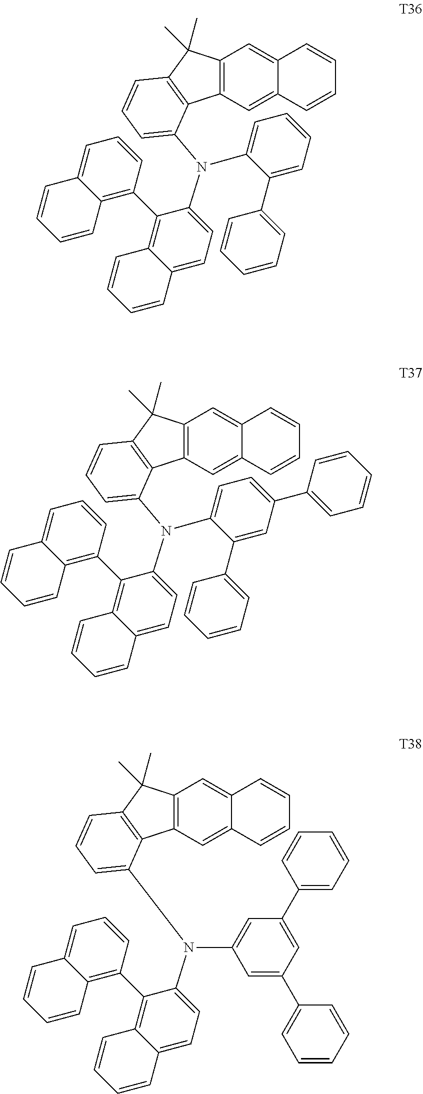 [SMILES: CC1(C)c2cc3ccccc3cc2-c2c(N(c3ccccc3-c3ccccc3)c3ccc4ccccc4c3-c3cccc4ccccc34)cccc21 ; CC1(C)c2cc3ccccc3cc2-c2c(N(c3ccc(-c4ccccc4)cc3-c3ccccc3)c3ccc4ccccc4c3-c3cccc4ccccc34)cccc21 ; CC1(C)c2cc3ccccc3cc2-c2c(N(c3cc(-c4ccccc4)cc(-c4ccccc4)c3)c3ccc4ccccc4c3-c3cccc4ccccc34)cccc21]

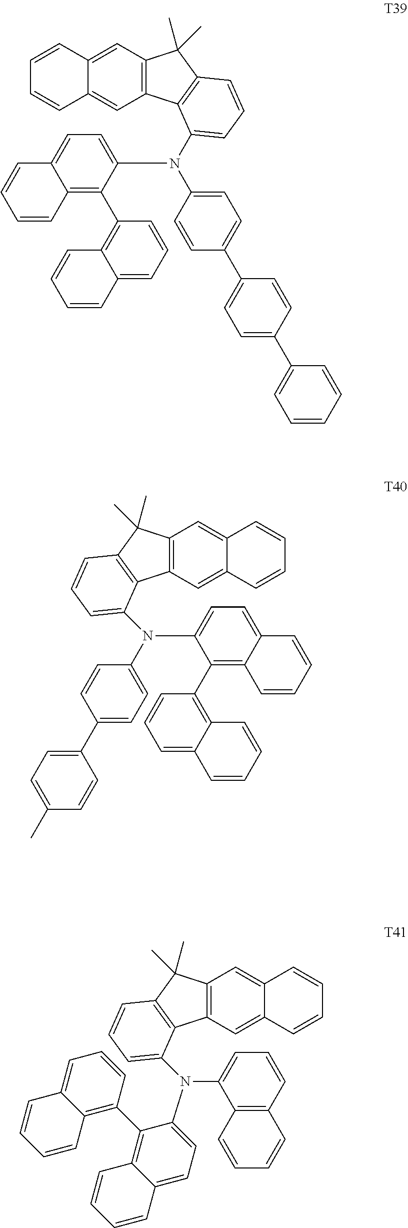 [SMILES: CC1(C)c2cc3ccccc3cc2-c2c(N(c3ccc(-c4ccc(-c5ccccc5)cc4)cc3)c3ccc4ccccc4c3-c3cccc4ccccc34)cccc21 ; Cc1ccc(-c2ccc(N(c3cccc4c3-c3cc5ccccc5cc3C4(C)C)c3ccc4ccccc4c3-c3cccc4ccccc34)cc2)cc1 ; CC1(C)c2cc3ccccc3cc2-c2c(N(c3ccc4ccccc4c3-c3cccc4ccccc34)c3cccc4ccccc34)cccc21]

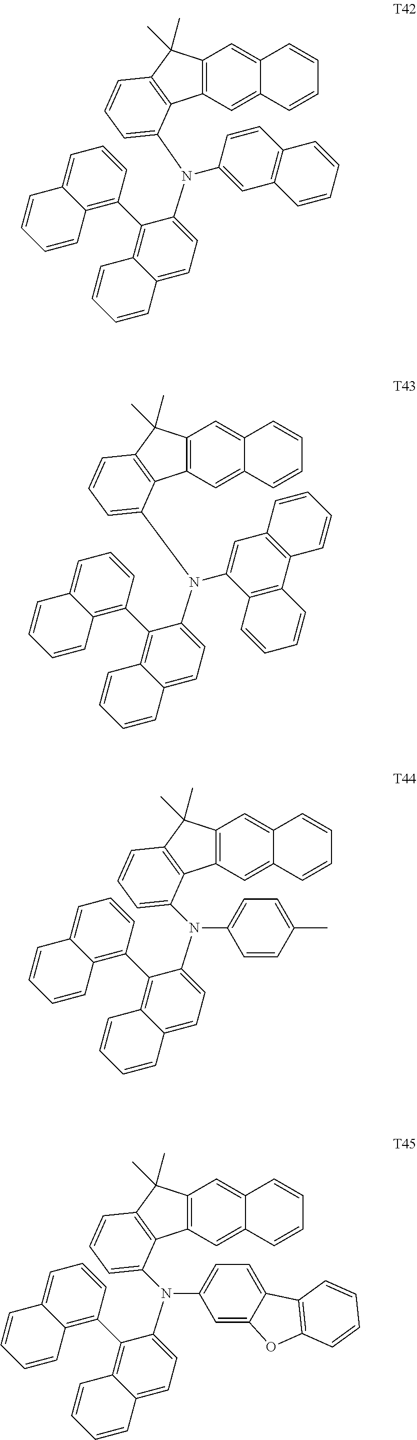 [SMILES: CC1(C)c2cc3ccccc3cc2-c2c(N(c3ccc4ccccc4c3)c3ccc4ccccc4c3-c3cccc4ccccc34)cccc21 ; CC1(C)c2cc3ccccc3cc2-c2c(N(c3ccc4ccccc4c3-c3cccc4ccccc34)c3cc4ccccc4c4ccccc34)cccc21 ; Cc1ccc(N(c2cccc3c2-c2cc4ccccc4cc2C3(C)C)c2ccc3ccccc3c2-c2cccc3ccccc23)cc1 ; CC1(C)c2cc3ccccc3cc2-c2c(N(c3ccc4c(c3)oc3ccccc34)c3ccc4ccccc4c3-c3cccc4ccccc34)cccc21]

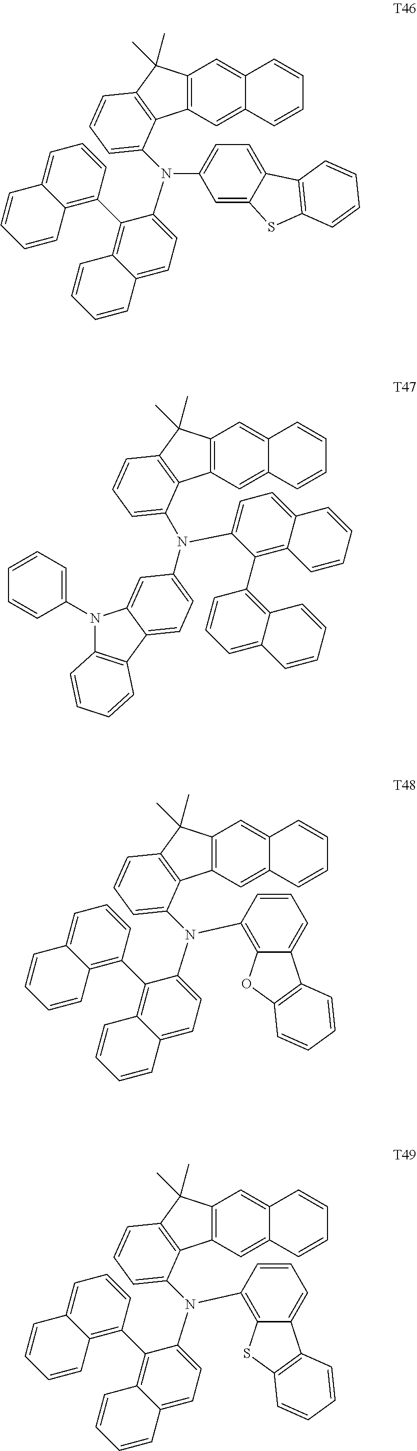 [SMILES: CC1(C)c2cc3ccccc3cc2-c2c(N(c3ccc4c(c3)sc3ccccc34)c3ccc4ccccc4c3-c3cccc4ccccc34)cccc21 ; CC1(C)c2cc3ccccc3cc2-c2c(N(c3ccc4c5ccccc5n(-c5ccccc5)c4c3)c3ccc4ccccc4c3-c3cccc4ccccc34)cccc21 ; CC1(C)c2cc3ccccc3cc2-c2c(N(c3ccc4ccccc4c3-c3cccc4ccccc34)c3cccc4c3oc3ccccc34)cccc21 ; CC1(C)c2cc3ccccc3cc2-c2c(N(c3ccc4ccccc4c3-c3cccc4ccccc34)c3cccc4c3sc3ccccc34)cccc21]

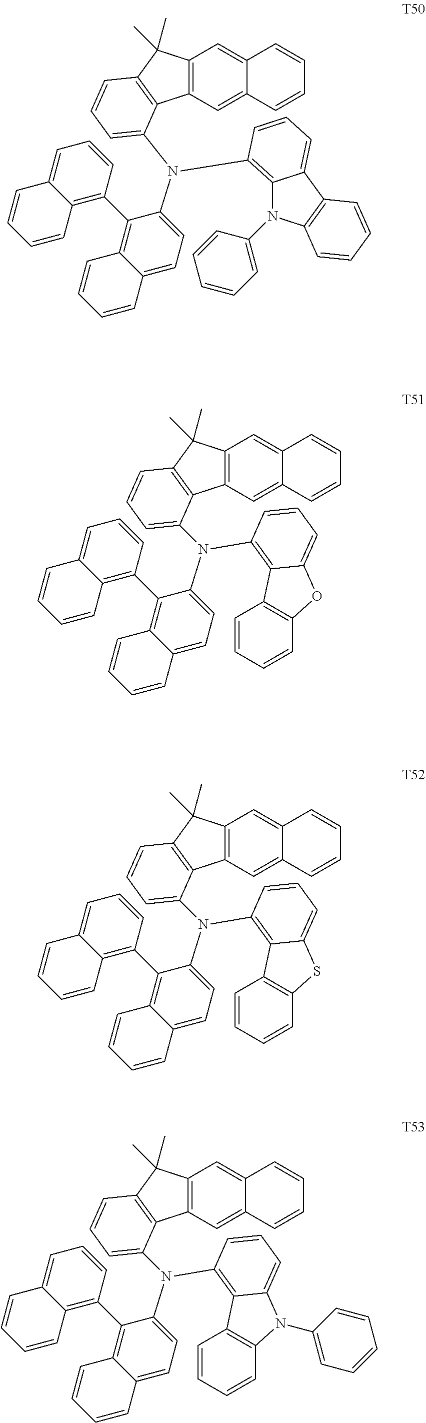 [SMILES: CC1(C)c2cc3ccccc3cc2-c2c(N(c3ccc4ccccc4c3-c3cccc4ccccc34)c3cccc4c5ccccc5n(-c5ccccc5)c34)cccc21 ; CC1(C)c2cc3ccccc3cc2-c2c(N(c3ccc4ccccc4c3-c3cccc4ccccc34)c3cccc4oc5ccccc5c34)cccc21 ; CC1(C)c2cc3ccccc3cc2-c2c(N(c3ccc4ccccc4c3-c3cccc4ccccc34)c3cccc4sc5ccccc5c34)cccc21 ; CC1(C)c2cc3ccccc3cc2-c2c(N(c3ccc4ccccc4c3-c3cccc4ccccc34)c3cccc4c3c3ccccc3n4-c3ccccc3)cccc21]

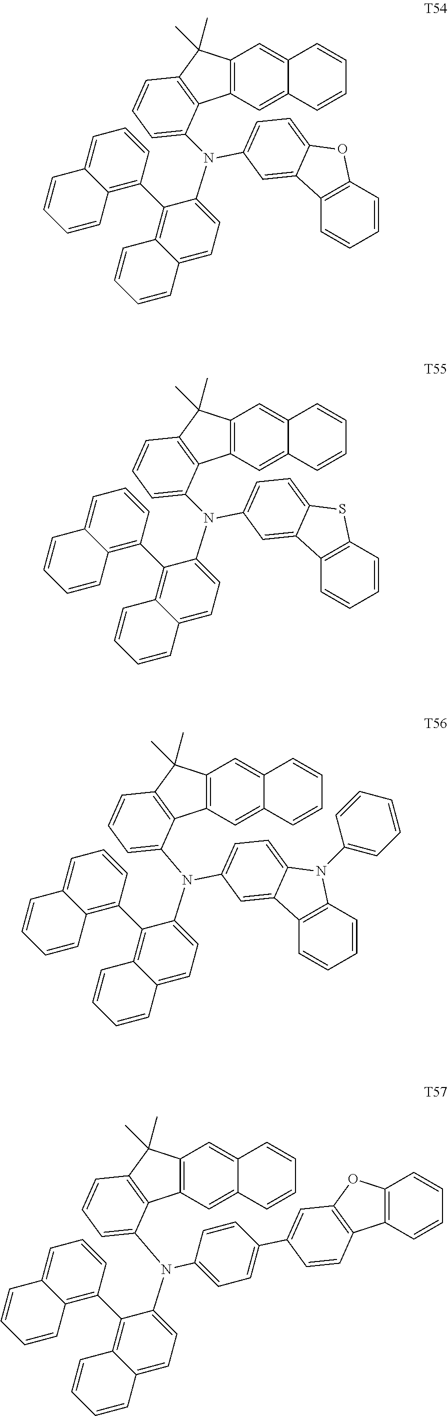 [SMILES: CC1(C)c2cc3ccccc3cc2-c2c(N(c3ccc4oc5ccccc5c4c3)c3ccc4ccccc4c3-c3cccc4ccccc34)cccc21 ; CC1(C)c2cc3ccccc3cc2-c2c(N(c3ccc4sc5ccccc5c4c3)c3ccc4ccccc4c3-c3cccc4ccccc34)cccc21 ; CC1(C)c2cc3ccccc3cc2-c2c(N(c3ccc4c(c3)c3ccccc3n4-c3ccccc3)c3ccc4ccccc4c3-c3cccc4ccccc34)cccc21 ; CC1(C)c2cc3ccccc3cc2-c2c(N(c3ccc(-c4ccc5c(c4)oc4ccccc45)cc3)c3ccc4ccccc4c3-c3cccc4ccccc34)cccc21]

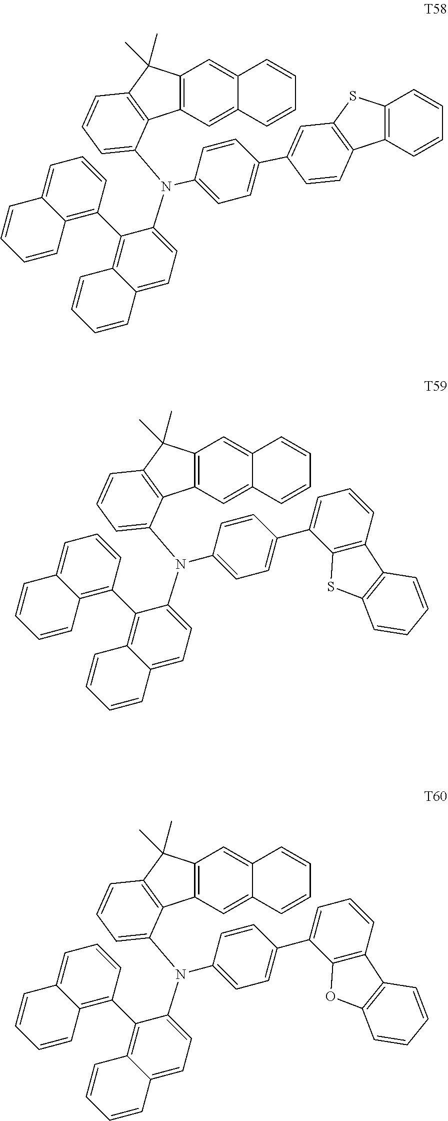 [SMILES: CC1(C)c2cc3ccccc3cc2-c2c(N(c3ccc(-c4ccc5c(c4)sc4ccccc45)cc3)c3ccc4ccccc4c3-c3cccc4ccccc34)cccc21 ; CC1(C)c2cc3ccccc3cc2-c2c(N(c3ccc(-c4cccc5c4sc4ccccc45)cc3)c3ccc4ccccc4c3-c3cccc4ccccc34)cccc21 ; CC1(C)c2cc3ccccc3cc2-c2c(N(c3ccc(-c4cccc5c4oc4ccccc45)cc3)c3ccc4ccccc4c3-c3cccc4ccccc34)cccc21]

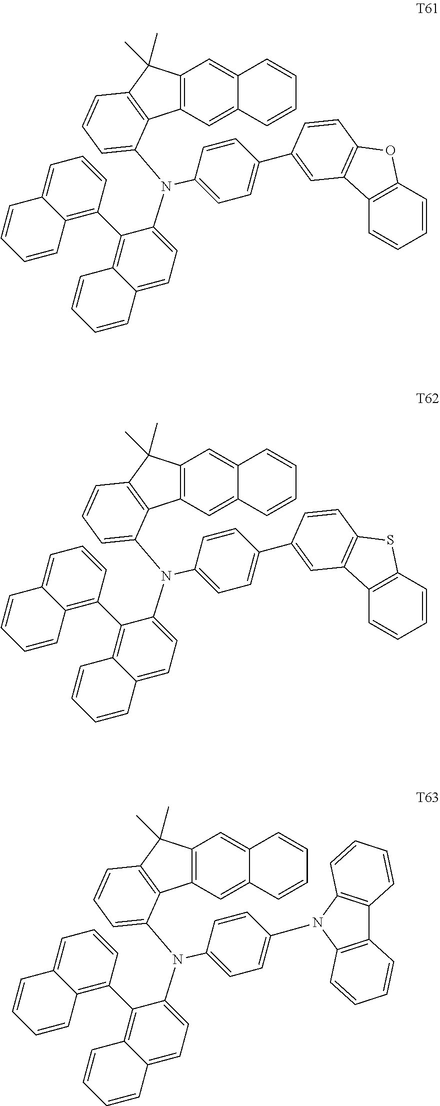 [SMILES: CC1(C)c2cc3ccccc3cc2-c2c(N(c3ccc(-c4ccc5oc6ccccc6c5c4)cc3)c3ccc4ccccc4c3-c3cccc4ccccc34)cccc21 ; CC1(C)c2cc3ccccc3cc2-c2c(N(c3ccc(-c4ccc5sc6ccccc6c5c4)cc3)c3ccc4ccccc4c3-c3cccc4ccccc34)cccc21 ; CC1(C)c2cc3ccccc3cc2-c2c(N(c3ccc(-n4c5ccccc5c5ccccc54)cc3)c3ccc4ccccc4c3-c3cccc4ccccc34)cccc21]

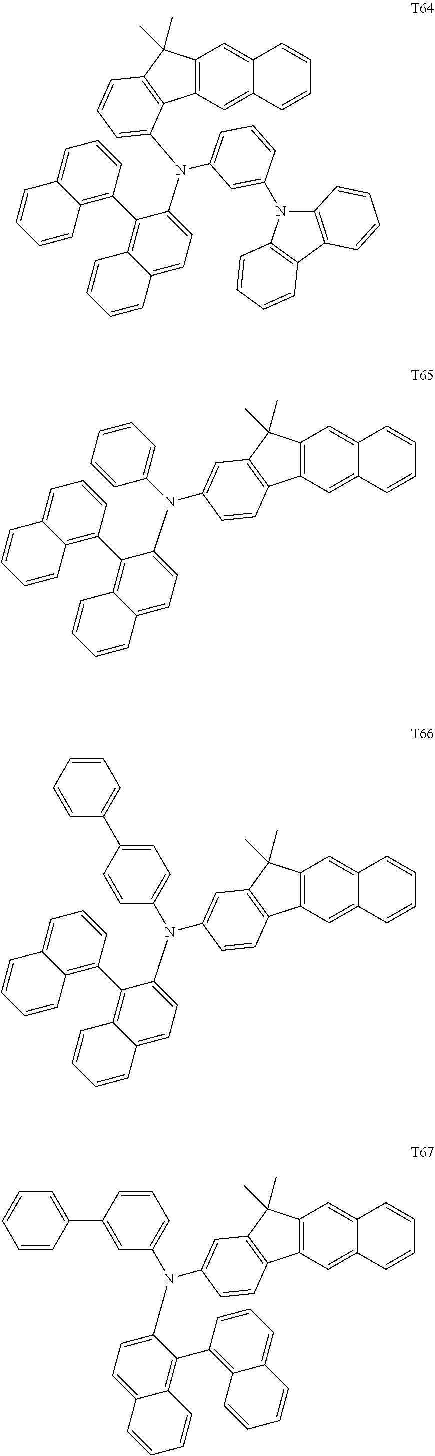 [SMILES: CC1(C)c2cc3ccccc3cc2-c2c(N(c3cccc(-n4c5ccccc5c5ccccc54)c3)c3ccc4ccccc4c3-c3cccc4ccccc34)cccc21 ; CC1(C)c2cc(N(c3ccccc3)c3ccc4ccccc4c3-c3cccc4ccccc34)ccc2-c2cc3ccccc3cc21 ; CC1(C)c2cc(N(c3ccc(-c4ccccc4)cc3)c3ccc4ccccc4c3-c3cccc4ccccc34)ccc2-c2cc3ccccc3cc21 ; CC1(C)c2cc(N(c3cccc(-c4ccccc4)c3)c3ccc4ccccc4c3-c3cccc4ccccc34)ccc2-c2cc3ccccc3cc21]

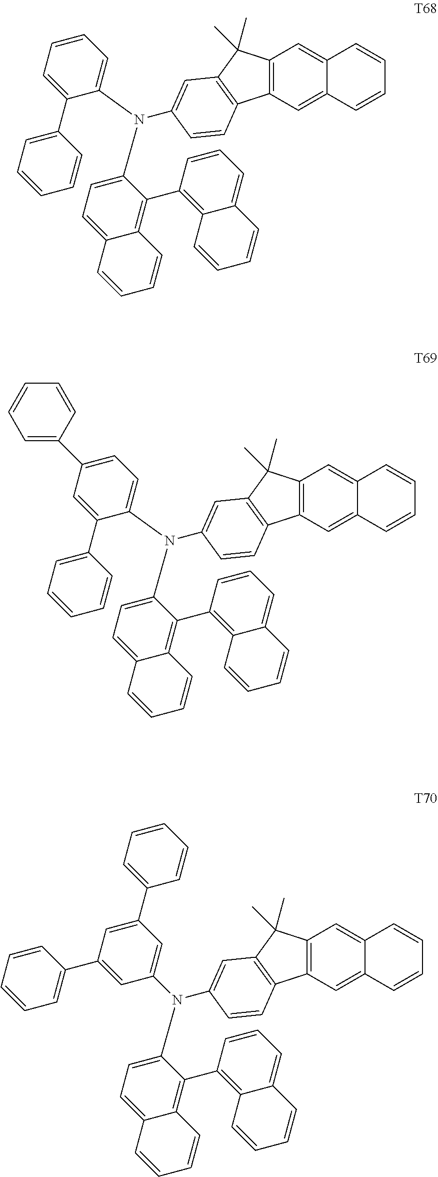 [SMILES: CC1(C)c2cc(N(c3ccccc3-c3ccccc3)c3ccc4ccccc4c3-c3cccc4ccccc34)ccc2-c2cc3ccccc3cc21 ; CC1(C)c2cc(N(c3ccc(-c4ccccc4)cc3-c3ccccc3)c3ccc4ccccc4c3-c3cccc4ccccc34)ccc2-c2cc3ccccc3cc21 ; CC1(C)c2cc(N(c3cc(-c4ccccc4)cc(-c4ccccc4)c3)c3ccc4ccccc4c3-c3cccc4ccccc34)ccc2-c2cc3ccccc3cc21]

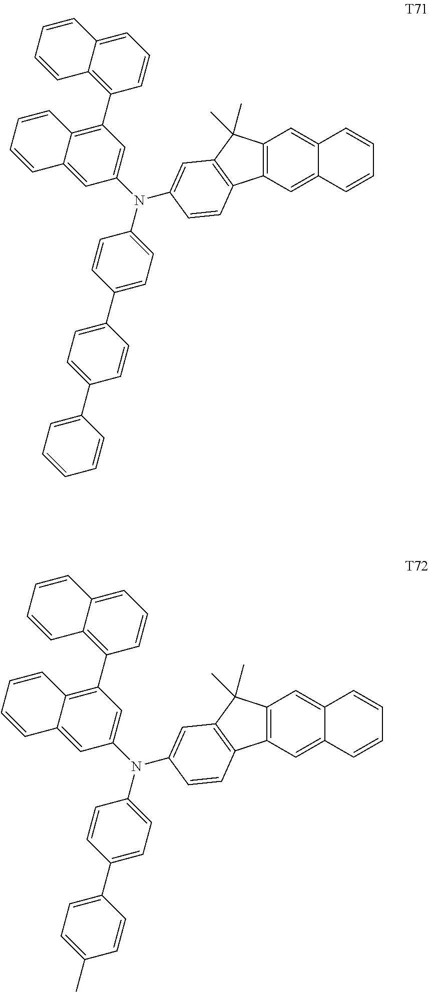 [SMILES: CC1(C)c2cc(N(c3ccc(-c4ccc(-c5ccccc5)cc4)cc3)c3cc(-c4cccc5ccccc45)c4ccccc4c3)ccc2-c2cc3ccccc3cc21 ; Cc1ccc(-c2ccc(N(c3ccc4c(c3)C(C)(C)c3cc5ccccc5cc3-4)c3cc(-c4cccc5ccccc45)c4ccccc4c3)cc2)cc1]

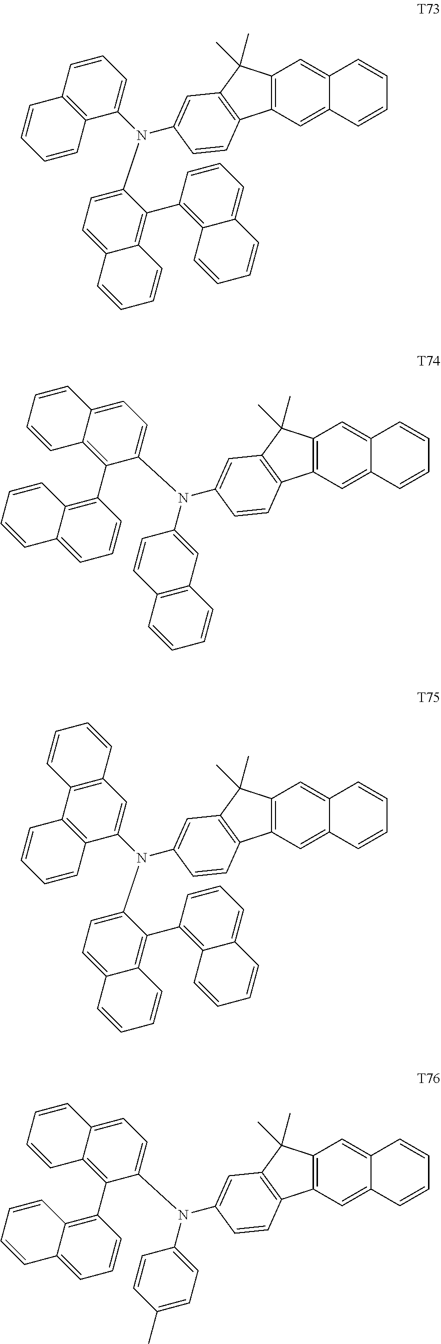 [SMILES: CC1(C)c2cc(N(c3ccc4ccccc4c3-c3cccc4ccccc34)c3cccc4ccccc34)ccc2-c2cc3ccccc3cc21 ; CC1(C)c2cc(N(c3ccc4ccccc4c3)c3ccc4ccccc4c3-c3cccc4ccccc34)ccc2-c2cc3ccccc3cc21 ; CC1(C)c2cc(N(c3ccc4ccccc4c3-c3cccc4ccccc34)c3cc4ccccc4c4ccccc34)ccc2-c2cc3ccccc3cc21 ; Cc1ccc(N(c2ccc3c(c2)C(C)(C)c2cc4ccccc4cc2-3)c2ccc3ccccc3c2-c2cccc3ccccc23)cc1]

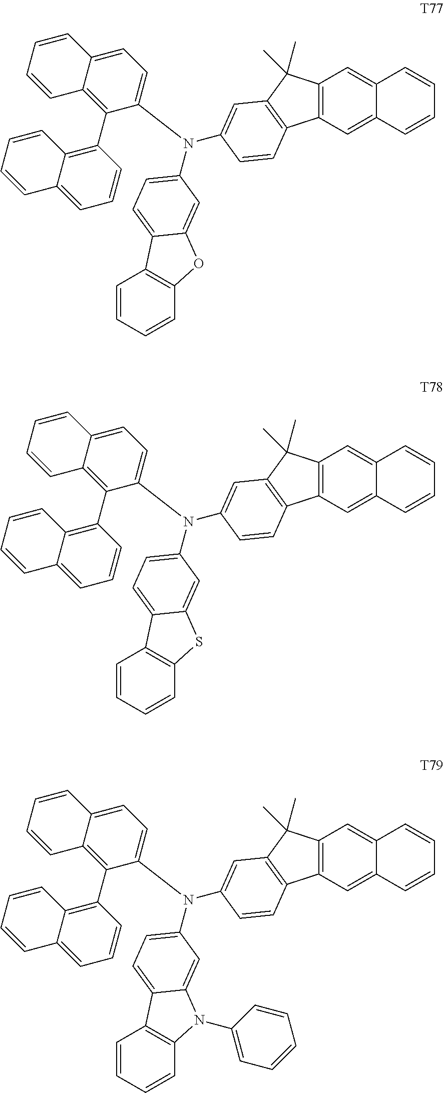 [SMILES: CC1(C)c2cc(N(c3ccc4c(c3)oc3ccccc34)c3ccc4ccccc4c3-c3cccc4ccccc34)ccc2-c2cc3ccccc3cc21 ; CC1(C)c2cc(N(c3ccc4c(c3)sc3ccccc34)c3ccc4ccccc4c3-c3cccc4ccccc34)ccc2-c2cc3ccccc3cc21 ; CC1(C)c2cc(N(c3ccc4c5ccccc5n(-c5ccccc5)c4c3)c3ccc4ccccc4c3-c3cccc4ccccc34)ccc2-c2cc3ccccc3cc21]

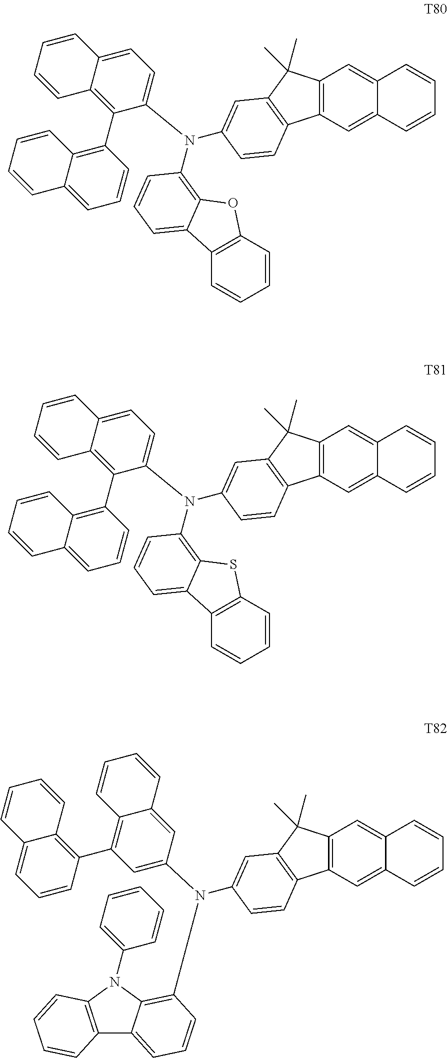 [SMILES: CC1(C)c2cc(N(c3ccc4ccccc4c3-c3cccc4ccccc34)c3cccc4c3oc3ccccc34)ccc2-c2cc3ccccc3cc21 ; CC1(C)c2cc(N(c3ccc4ccccc4c3-c3cccc4ccccc34)c3cccc4c3sc3ccccc34)ccc2-c2cc3ccccc3cc21 ; CC1(C)c2cc(N(c3cc(-c4cccc5ccccc45)c4ccccc4c3)c3cccc4c5ccccc5n(-c5ccccc5)c34)ccc2-c2cc3ccccc3cc21]

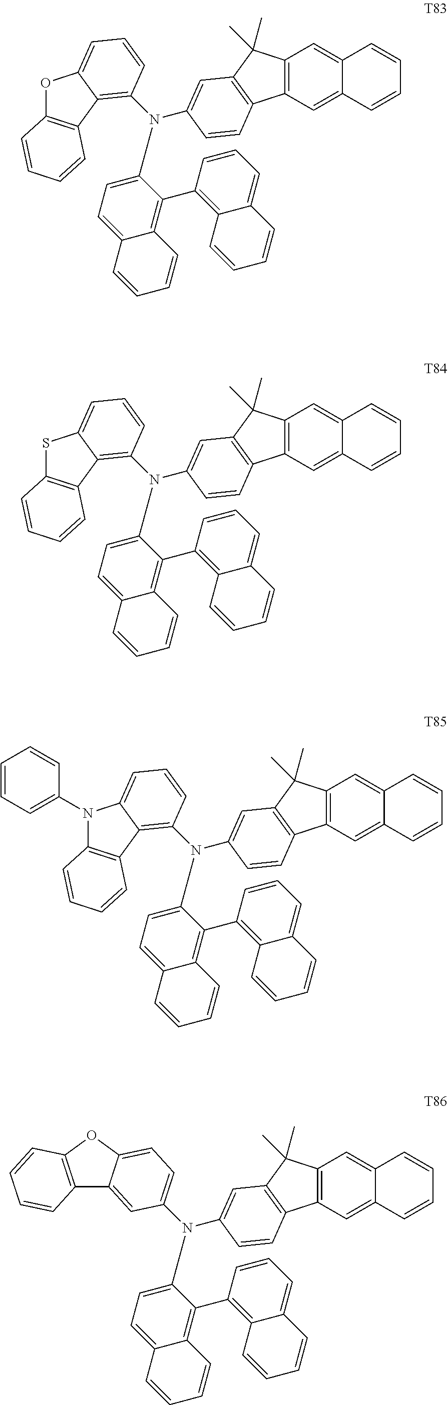 [SMILES: CC1(C)c2cc(N(c3ccc4ccccc4c3-c3cccc4ccccc34)c3cccc4oc5ccccc5c34)ccc2-c2cc3ccccc3cc21 ; CC1(C)c2cc(N(c3ccc4ccccc4c3-c3cccc4ccccc34)c3cccc4sc5ccccc5c34)ccc2-c2cc3ccccc3cc21 ; CC1(C)c2cc(N(c3ccc4ccccc4c3-c3cccc4ccccc34)c3cccc4c3c3ccccc3n4-c3ccccc3)ccc2-c2cc3ccccc3cc21 ; CC1(C)c2cc(N(c3ccc4oc5ccccc5c4c3)c3ccc4ccccc4c3-c3cccc4ccccc34)ccc2-c2cc3ccccc3cc21]

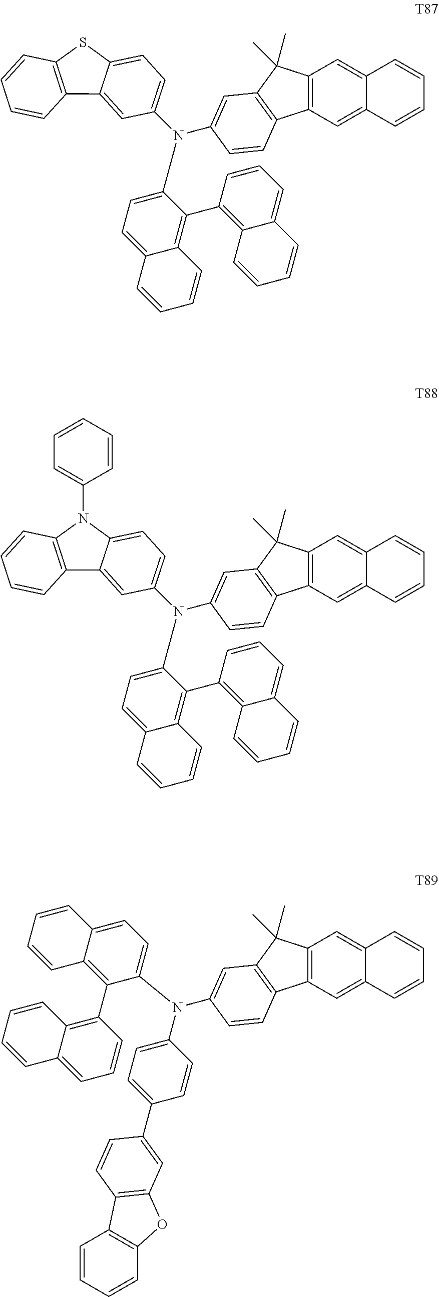 [SMILES: CC1(C)c2cc(N(c3ccc4sc5ccccc5c4c3)c3ccc4ccccc4c3-c3cccc4ccccc34)ccc2-c2cc3ccccc3cc21 ; CC1(C)c2cc(N(c3ccc4c(c3)c3ccccc3n4-c3ccccc3)c3ccc4ccccc4c3-c3cccc4ccccc34)ccc2-c2cc3ccccc3cc21 ; CC1(C)c2cc(N(c3ccc(-c4ccc5c(c4)oc4ccccc45)cc3)c3ccc4ccccc4c3-c3cccc4ccccc34)ccc2-c2cc3ccccc3cc21]

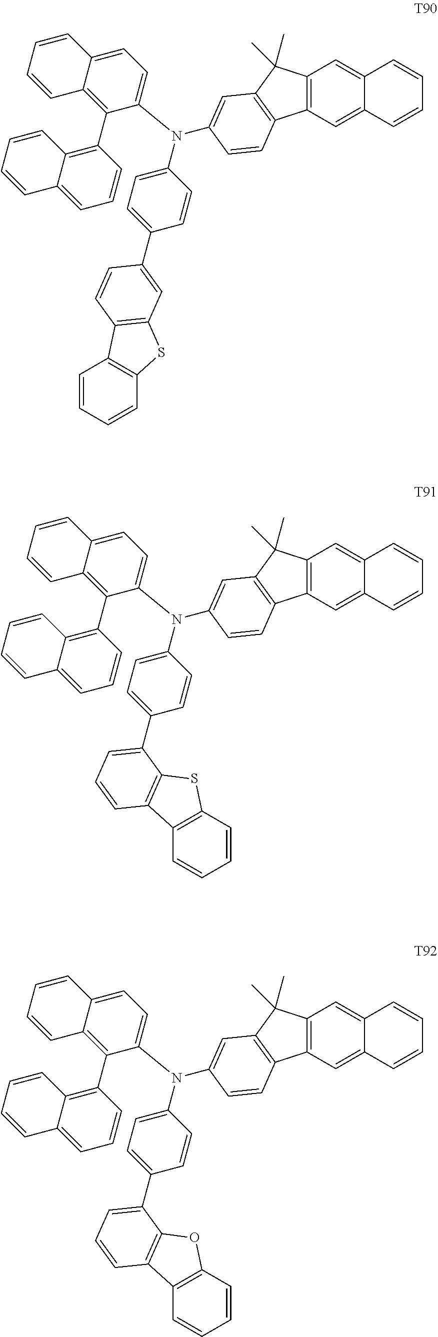 [SMILES: CC1(C)c2cc(N(c3ccc(-c4ccc5c(c4)sc4ccccc45)cc3)c3ccc4ccccc4c3-c3cccc4ccccc34)ccc2-c2cc3ccccc3cc21 ; CC1(C)c2cc(N(c3ccc(-c4cccc5c4sc4ccccc45)cc3)c3ccc4ccccc4c3-c3cccc4ccccc34)ccc2-c2cc3ccccc3cc21 ; CC1(C)c2cc(N(c3ccc(-c4cccc5c4oc4ccccc45)cc3)c3ccc4ccccc4c3-c3cccc4ccccc34)ccc2-c2cc3ccccc3cc21]

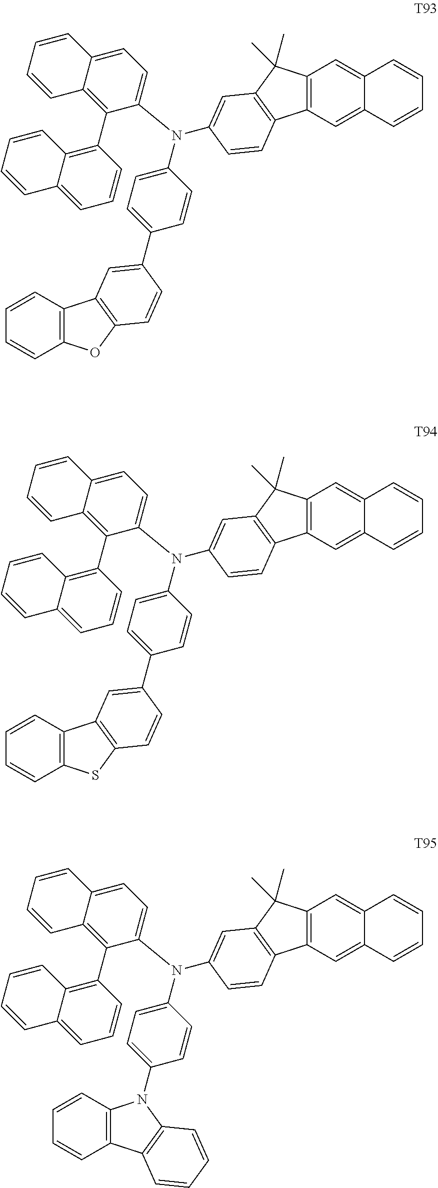 [SMILES: CC1(C)c2cc(N(c3ccc(-c4ccc5oc6ccccc6c5c4)cc3)c3ccc4ccccc4c3-c3cccc4ccccc34)ccc2-c2cc3ccccc3cc21 ; CC1(C)c2cc(N(c3ccc(-c4ccc5sc6ccccc6c5c4)cc3)c3ccc4ccccc4c3-c3cccc4ccccc34)ccc2-c2cc3ccccc3cc21 ; CC1(C)c2cc(N(c3ccc(-n4c5ccccc5c5ccccc54)cc3)c3ccc4ccccc4c3-c3cccc4ccccc34)ccc2-c2cc3ccccc3cc21]

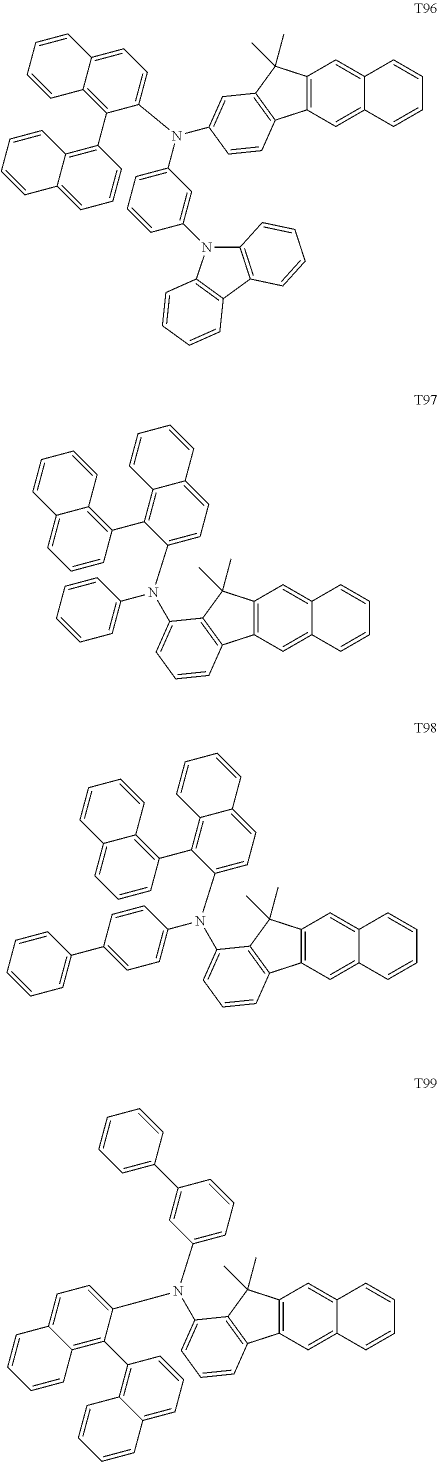 [SMILES: CC1(C)c2cc(N(c3cccc(-n4c5ccccc5c5ccccc54)c3)c3ccc4ccccc4c3-c3cccc4ccccc34)ccc2-c2cc3ccccc3cc21 ; CC1(C)c2cc3ccccc3cc2-c2cccc(N(c3ccccc3)c3ccc4ccccc4c3-c3cccc4ccccc34)c21 ; CC1(C)c2cc3ccccc3cc2-c2cccc(N(c3ccc(-c4ccccc4)cc3)c3ccc4ccccc4c3-c3cccc4ccccc34)c21 ; CC1(C)c2cc3ccccc3cc2-c2cccc(N(c3cccc(-c4ccccc4)c3)c3ccc4ccccc4c3-c3cccc4ccccc34)c21]

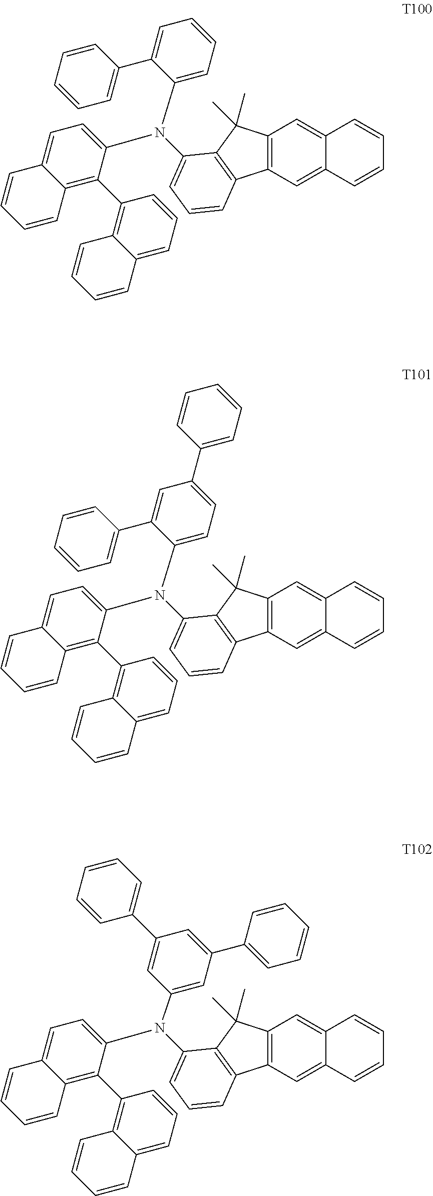 [SMILES: CC1(C)c2cc3ccccc3cc2-c2cccc(N(c3ccccc3-c3ccccc3)c3ccc4ccccc4c3-c3cccc4ccccc34)c21 ; CC1(C)c2cc3ccccc3cc2-c2cccc(N(c3ccc(-c4ccccc4)cc3-c3ccccc3)c3ccc4ccccc4c3-c3cccc4ccccc34)c21 ; CC1(C)c2cc3ccccc3cc2-c2cccc(N(c3cc(-c4ccccc4)cc(-c4ccccc4)c3)c3ccc4ccccc4c3-c3cccc4ccccc34)c21]

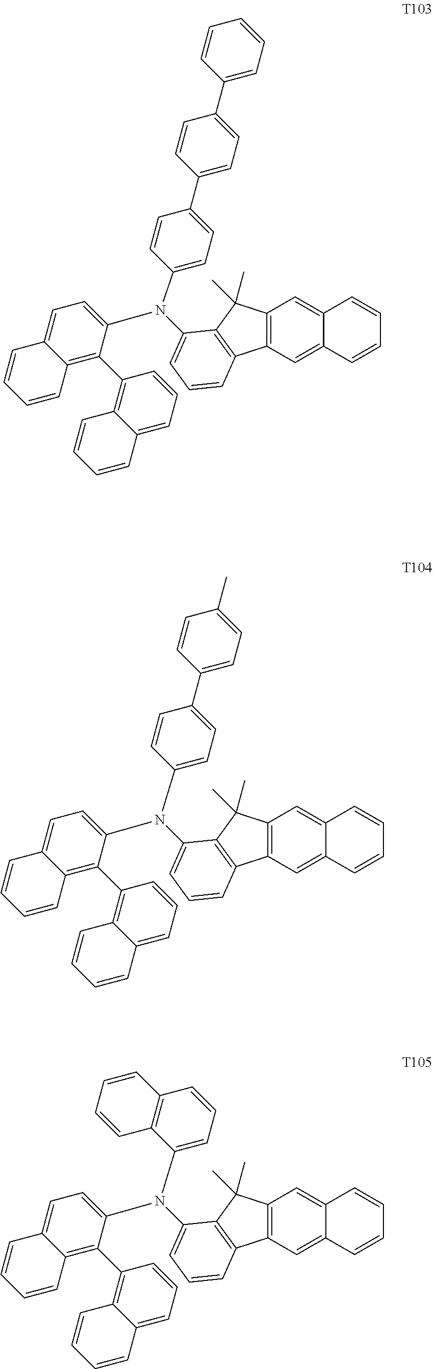 [SMILES: CC1(C)c2cc3ccccc3cc2-c2cccc(N(c3ccc(-c4ccc(-c5ccccc5)cc4)cc3)c3ccc4ccccc4c3-c3cccc4ccccc34)c21 ; Cc1ccc(-c2ccc(N(c3cccc4c3C(C)(C)c3cc5ccccc5cc3-4)c3ccc4ccccc4c3-c3cccc4ccccc34)cc2)cc1 ; CC1(C)c2cc3ccccc3cc2-c2cccc(N(c3ccc4ccccc4c3-c3cccc4ccccc34)c3cccc4ccccc34)c21]

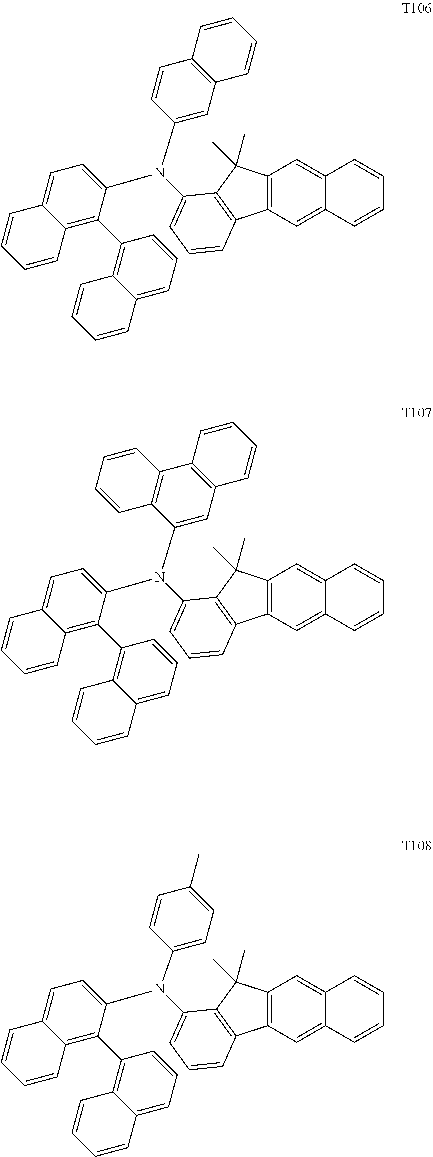 [SMILES: CC1(C)c2cc3ccccc3cc2-c2cccc(N(c3ccc4ccccc4c3)c3ccc4ccccc4c3-c3cccc4ccccc34)c21 ; CC1(C)c2cc3ccccc3cc2-c2cccc(N(c3ccc4ccccc4c3-c3cccc4ccccc34)c3cc4ccccc4c4ccccc34)c21 ; Cc1ccc(N(c2cccc3c2C(C)(C)c2cc4ccccc4cc2-3)c2ccc3ccccc3c2-c2cccc3ccccc23)cc1]

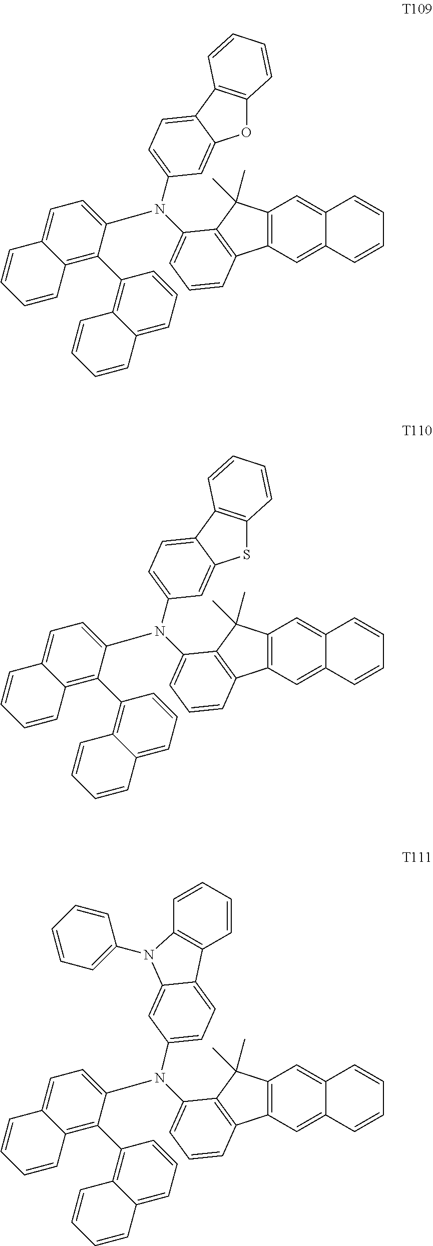 [SMILES: CC1(C)c2cc3ccccc3cc2-c2cccc(N(c3ccc4c(c3)oc3ccccc34)c3ccc4ccccc4c3-c3cccc4ccccc34)c21 ; CC1(C)c2cc3ccccc3cc2-c2cccc(N(c3ccc4c(c3)sc3ccccc34)c3ccc4ccccc4c3-c3cccc4ccccc34)c21 ; CC1(C)c2cc3ccccc3cc2-c2cccc(N(c3ccc4c5ccccc5n(-c5ccccc5)c4c3)c3ccc4ccccc4c3-c3cccc4ccccc34)c21]

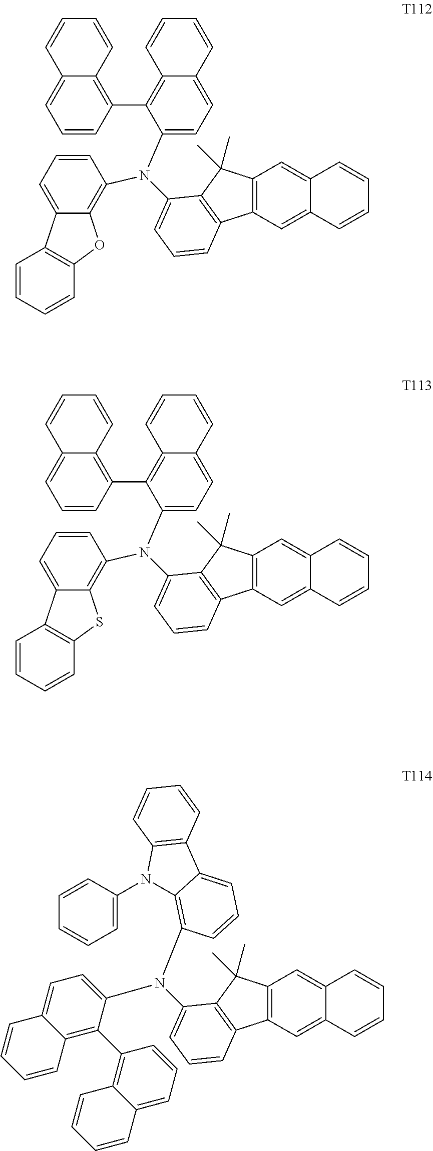 [SMILES: CC1(C)c2cc3ccccc3cc2-c2cccc(N(c3ccc4ccccc4c3-c3cccc4ccccc34)c3cccc4c3oc3ccccc34)c21 ; CC1(C)c2cc3ccccc3cc2-c2cccc(N(c3ccc4ccccc4c3-c3cccc4ccccc34)c3cccc4c3sc3ccccc34)c21 ; CC1(C)c2cc3ccccc3cc2-c2cccc(N(c3ccc4ccccc4c3-c3cccc4ccccc34)c3cccc4c5ccccc5n(-c5ccccc5)c34)c21]

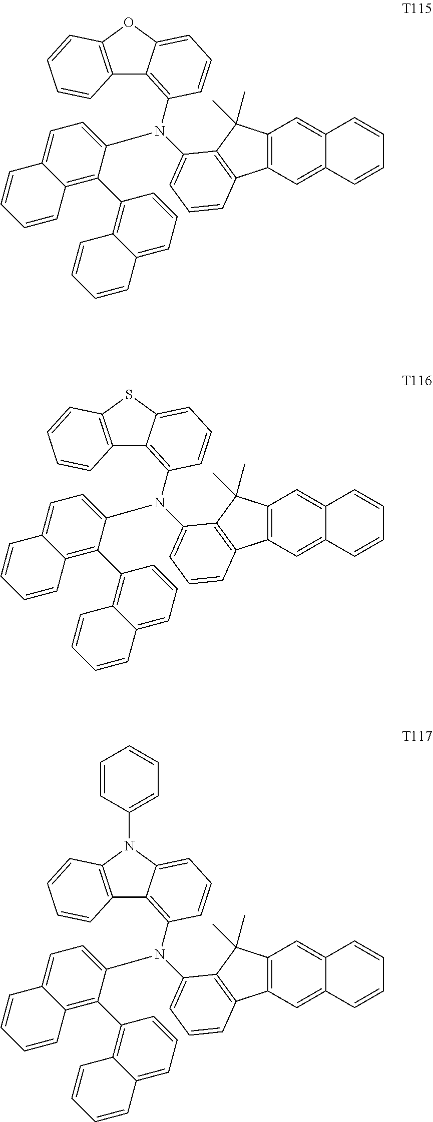 [SMILES: CC1(C)c2cc3ccccc3cc2-c2cccc(N(c3ccc4ccccc4c3-c3cccc4ccccc34)c3cccc4oc5ccccc5c34)c21 ; CC1(C)c2cc3ccccc3cc2-c2cccc(N(c3ccc4ccccc4c3-c3cccc4ccccc34)c3cccc4sc5ccccc5c34)c21 ; CC1(C)c2cc3ccccc3cc2-c2cccc(N(c3ccc4ccccc4c3-c3cccc4ccccc34)c3cccc4c3c3ccccc3n4-c3ccccc3)c21]

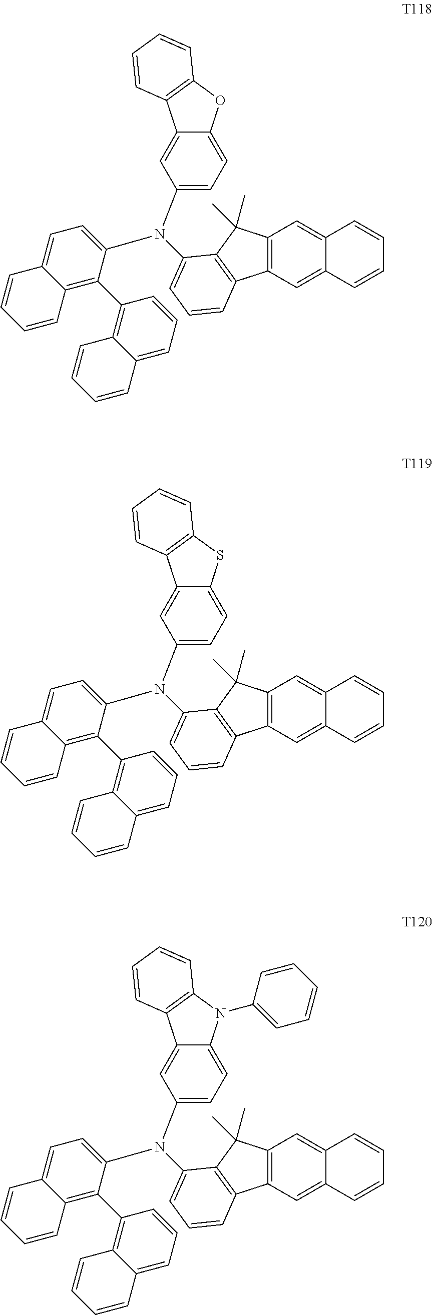 [SMILES: CC1(C)c2cc3ccccc3cc2-c2cccc(N(c3ccc4oc5ccccc5c4c3)c3ccc4ccccc4c3-c3cccc4ccccc34)c21 ; CC1(C)c2cc3ccccc3cc2-c2cccc(N(c3ccc4sc5ccccc5c4c3)c3ccc4ccccc4c3-c3cccc4ccccc34)c21 ; CC1(C)c2cc3ccccc3cc2-c2cccc(N(c3ccc4c(c3)c3ccccc3n4-c3ccccc3)c3ccc4ccccc4c3-c3cccc4ccccc34)c21]

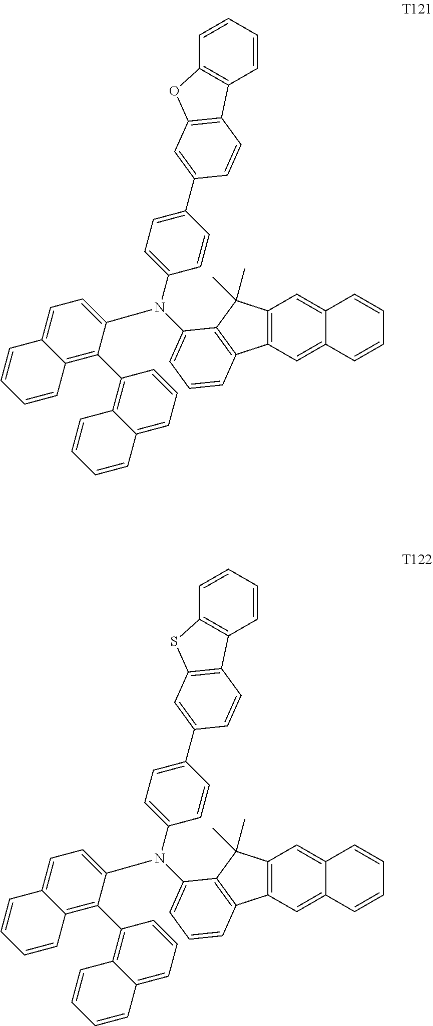 [SMILES: CC1(C)c2cc3ccccc3cc2-c2cccc(N(c3ccc(-c4ccc5c(c4)oc4ccccc45)cc3)c3ccc4ccccc4c3-c3cccc4ccccc34)c21 ; CC1(C)c2cc3ccccc3cc2-c2cccc(N(c3ccc(-c4ccc5c(c4)sc4ccccc45)cc3)c3ccc4ccccc4c3-c3cccc4ccccc34)c21]

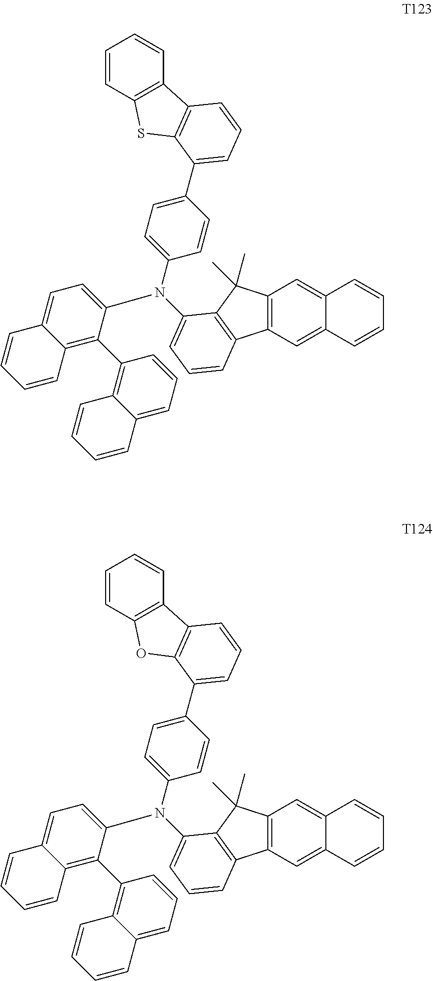 [SMILES: CC1(C)c2cc3ccccc3cc2-c2cccc(N(c3ccc(-c4cccc5c4sc4ccccc45)cc3)c3ccc4ccccc4c3-c3cccc4ccccc34)c21 ; CC1(C)c2cc3ccccc3cc2-c2cccc(N(c3ccc(-c4cccc5c4oc4ccccc45)cc3)c3ccc4ccccc4c3-c3cccc4ccccc34)c21]

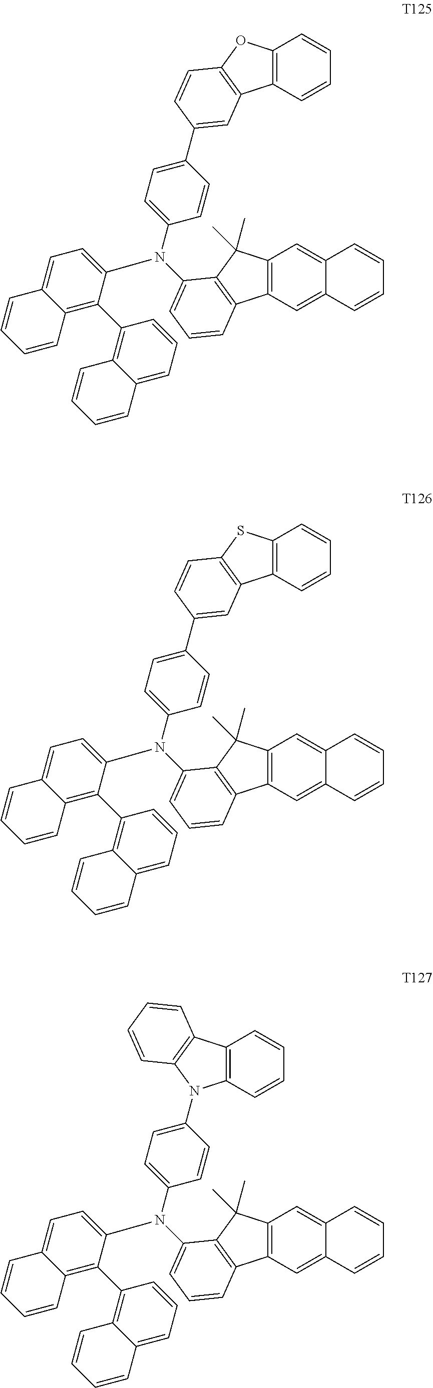 [SMILES: CC1(C)c2cc3ccccc3cc2-c2cccc(N(c3ccc(-c4ccc5oc6ccccc6c5c4)cc3)c3ccc4ccccc4c3-c3cccc4ccccc34)c21 ; CC1(C)c2cc3ccccc3cc2-c2cccc(N(c3ccc(-c4ccc5sc6ccccc6c5c4)cc3)c3ccc4ccccc4c3-c3cccc4ccccc34)c21 ; CC1(C)c2cc3ccccc3cc2-c2cccc(N(c3ccc(-n4c5ccccc5c5ccccc54)cc3)c3ccc4ccccc4c3-c3cccc4ccccc34)c21]

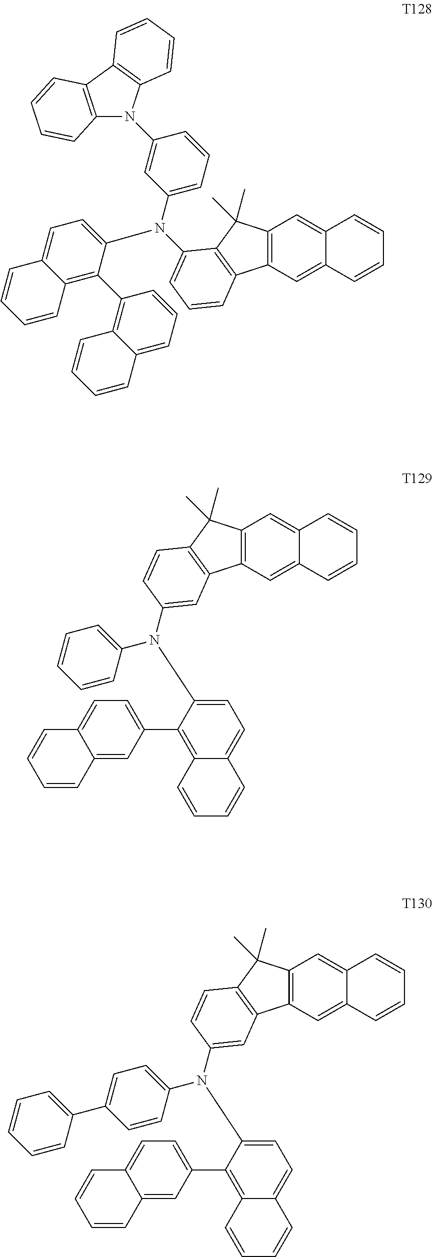 [SMILES: CC1(C)c2cc3ccccc3cc2-c2cccc(N(c3cccc(-n4c5ccccc5c5ccccc54)c3)c3ccc4ccccc4c3-c3cccc4ccccc34)c21 ; CC1(C)c2ccc(N(c3ccccc3)c3ccc4ccccc4c3-c3ccc4ccccc4c3)cc2-c2cc3ccccc3cc21 ; CC1(C)c2ccc(N(c3ccc(-c4ccccc4)cc3)c3ccc4ccccc4c3-c3ccc4ccccc4c3)cc2-c2cc3ccccc3cc21]

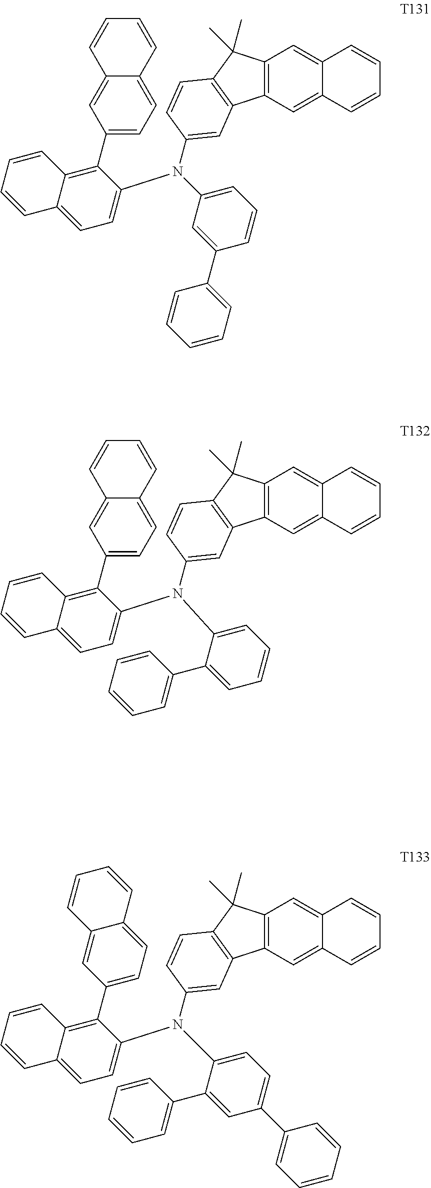 [SMILES: CC1(C)c2ccc(N(c3cccc(-c4ccccc4)c3)c3ccc4ccccc4c3-c3ccc4ccccc4c3)cc2-c2cc3ccccc3cc21 ; CC1(C)c2ccc(N(c3ccccc3-c3ccccc3)c3ccc4ccccc4c3-c3ccc4ccccc4c3)cc2-c2cc3ccccc3cc21 ; CC1(C)c2ccc(N(c3ccc(-c4ccccc4)cc3-c3ccccc3)c3ccc4ccccc4c3-c3ccc4ccccc4c3)cc2-c2cc3ccccc3cc21]

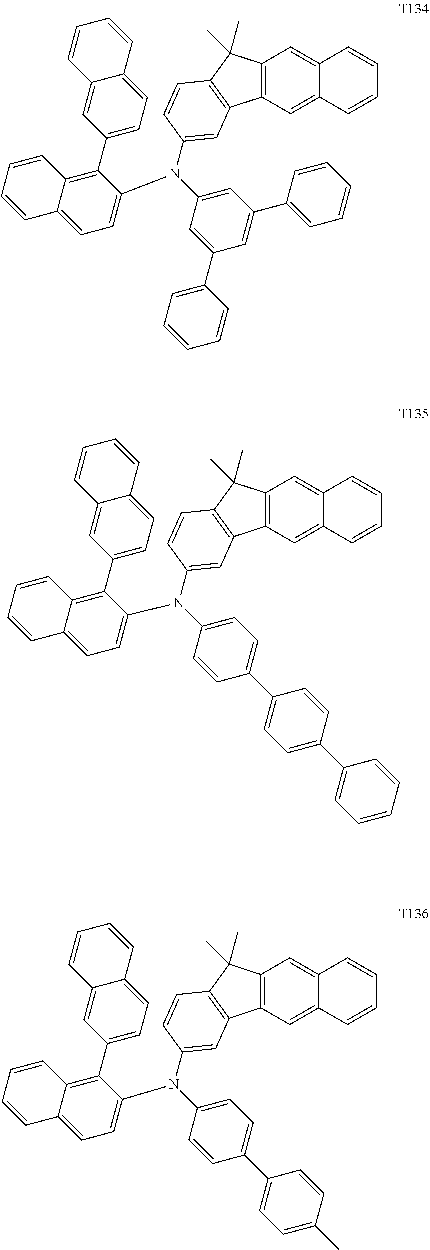 [SMILES: CC1(C)c2ccc(N(c3cc(-c4ccccc4)cc(-c4ccccc4)c3)c3ccc4ccccc4c3-c3ccc4ccccc4c3)cc2-c2cc3ccccc3cc21 ; CC1(C)c2ccc(N(c3ccc(-c4ccc(-c5ccccc5)cc4)cc3)c3ccc4ccccc4c3-c3ccc4ccccc4c3)cc2-c2cc3ccccc3cc21 ; Cc1ccc(-c2ccc(N(c3ccc4c(c3)-c3cc5ccccc5cc3C4(C)C)c3ccc4ccccc4c3-c3ccc4ccccc4c3)cc2)cc1]

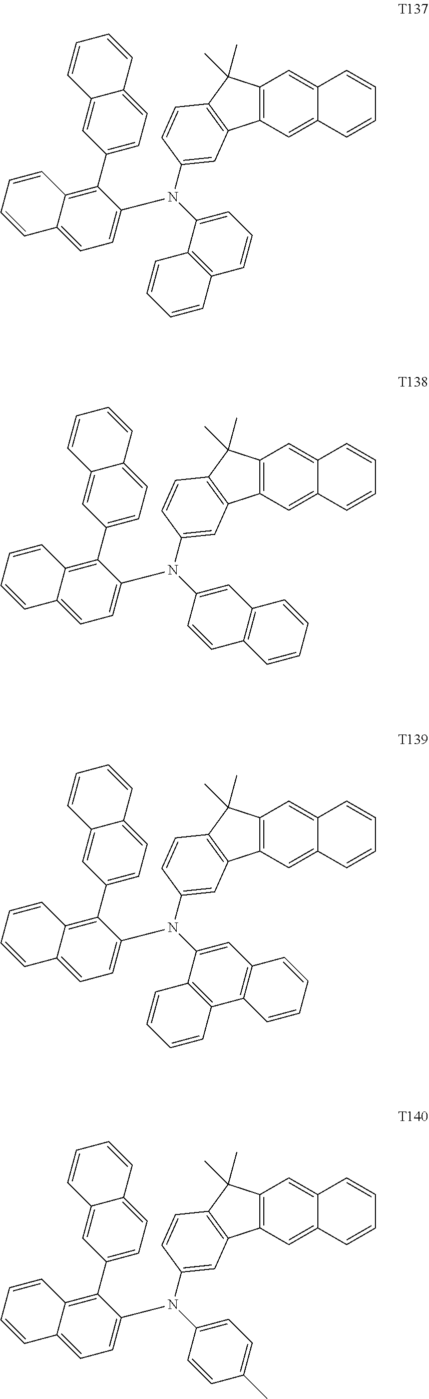 [SMILES: CC1(C)c2ccc(N(c3ccc4ccccc4c3-c3ccc4ccccc4c3)c3cccc4ccccc34)cc2-c2cc3ccccc3cc21 ; CC1(C)c2ccc(N(c3ccc4ccccc4c3)c3ccc4ccccc4c3-c3ccc4ccccc4c3)cc2-c2cc3ccccc3cc21 ; CC1(C)c2ccc(N(c3ccc4ccccc4c3-c3ccc4ccccc4c3)c3cc4ccccc4c4ccccc34)cc2-c2cc3ccccc3cc21 ; Cc1ccc(N(c2ccc3c(c2)-c2cc4ccccc4cc2C3(C)C)c2ccc3ccccc3c2-c2ccc3ccccc3c2)cc1]

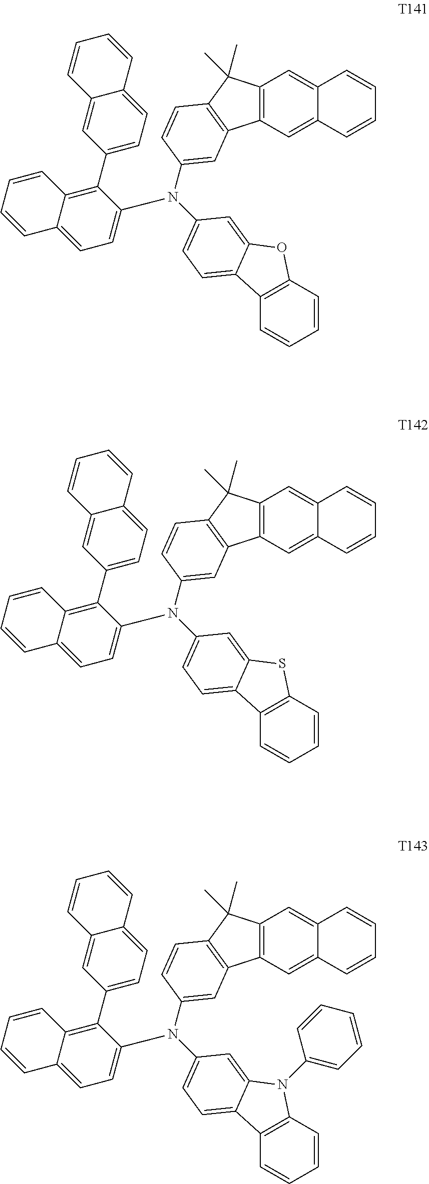 [SMILES: CC1(C)c2ccc(N(c3ccc4c(c3)oc3ccccc34)c3ccc4ccccc4c3-c3ccc4ccccc4c3)cc2-c2cc3ccccc3cc21 ; CC1(C)c2ccc(N(c3ccc4c(c3)sc3ccccc34)c3ccc4ccccc4c3-c3ccc4ccccc4c3)cc2-c2cc3ccccc3cc21 ; CC1(C)c2ccc(N(c3ccc4c5ccccc5n(-c5ccccc5)c4c3)c3ccc4ccccc4c3-c3ccc4ccccc4c3)cc2-c2cc3ccccc3cc21]

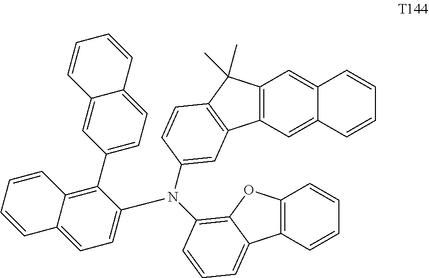 [SMILES: CC1(C)c2ccc(N(c3ccc4ccccc4c3-c3ccc4ccccc4c3)c3cccc4c3oc3ccccc34)cc2-c2cc3ccccc3cc21]

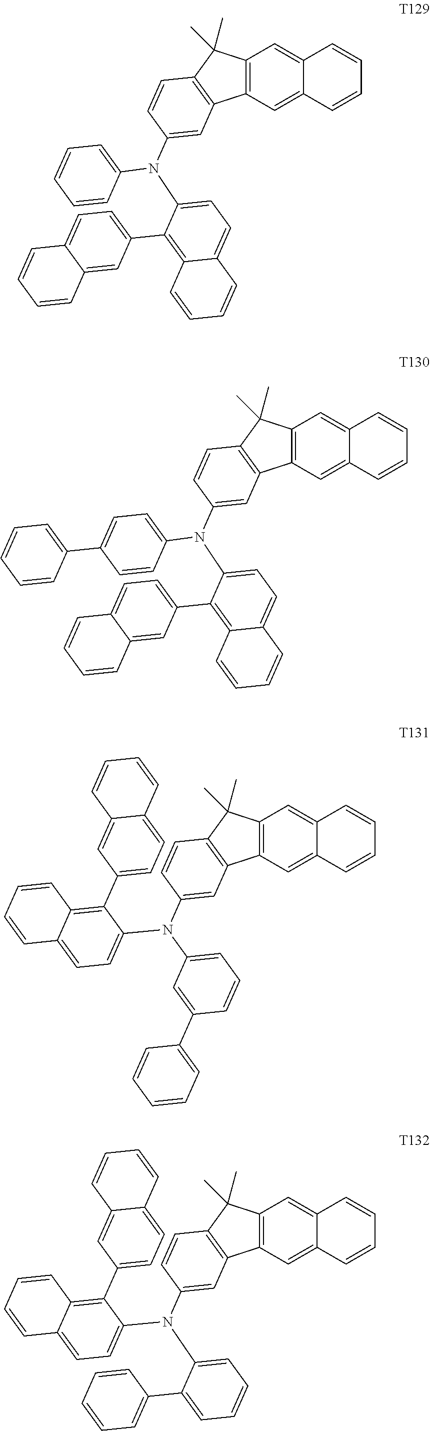

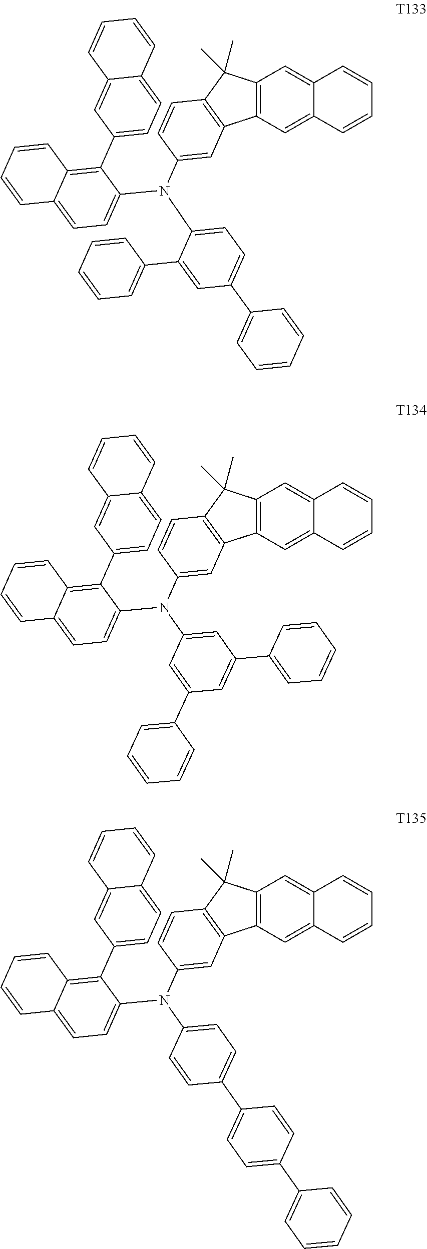

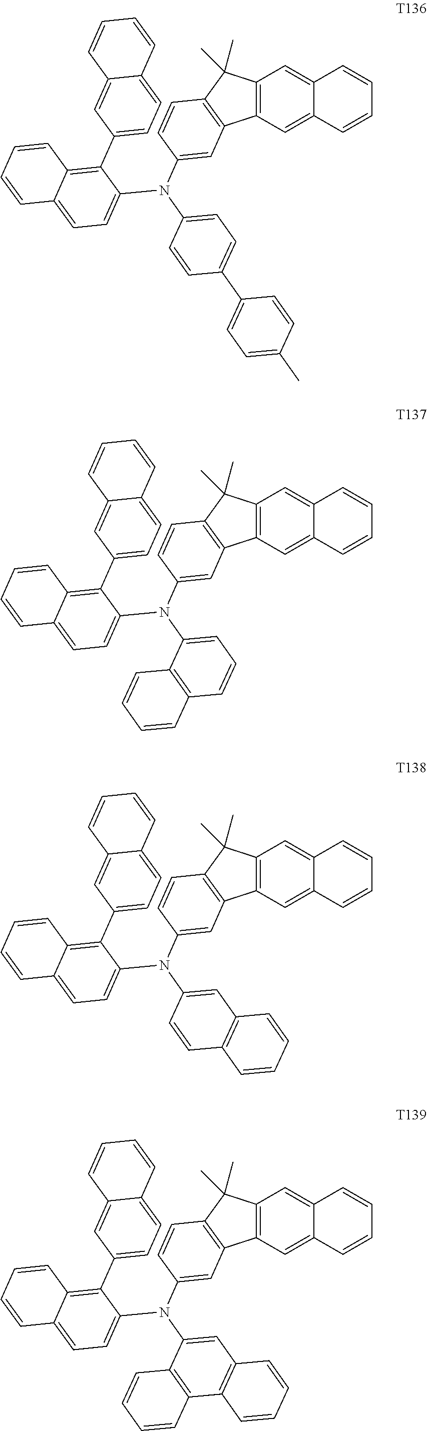 [SMILES: Cc1ccc(-c2ccc(N(c3ccc4c(c3)-c3cc5ccccc5cc3C4(C)C)c3ccc4ccccc4c3-c3ccc4ccccc4c3)cc2)cc1 ; CC1(C)c2ccc(N(c3ccc4ccccc4c3-c3ccc4ccccc4c3)c3cccc4ccccc34)cc2-c2cc3ccccc3cc21 ; CC1(C)c2ccc(N(c3ccc4ccccc4c3)c3ccc4ccccc4c3-c3ccc4ccccc4c3)cc2-c2cc3ccccc3cc21 ; CC1(C)c2ccc(N(c3ccc4ccccc4c3-c3ccc4ccccc4c3)c3cc4ccccc4c4ccccc34)cc2-c2cc3ccccc3cc21]

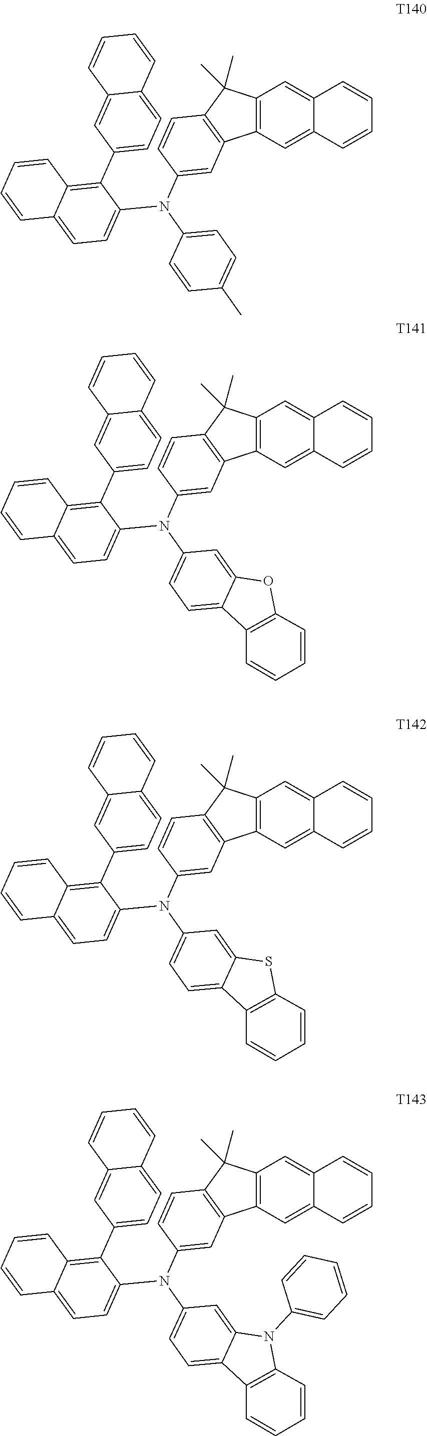 [SMILES: Cc1ccc(N(c2ccc3c(c2)-c2cc4ccccc4cc2C3(C)C)c2ccc3ccccc3c2-c2ccc3ccccc3c2)cc1 ; CC1(C)c2ccc(N(c3ccc4c(c3)oc3ccccc34)c3ccc4ccccc4c3-c3ccc4ccccc4c3)cc2-c2cc3ccccc3cc21 ; CC1(C)c2ccc(N(c3ccc4c(c3)sc3ccccc34)c3ccc4ccccc4c3-c3ccc4ccccc4c3)cc2-c2cc3ccccc3cc21 ; CC1(C)c2ccc(N(c3ccc4c5ccccc5n(-c5ccccc5)c4c3)c3ccc4ccccc4c3-c3ccc4ccccc4c3)cc2-c2cc3ccccc3cc21]

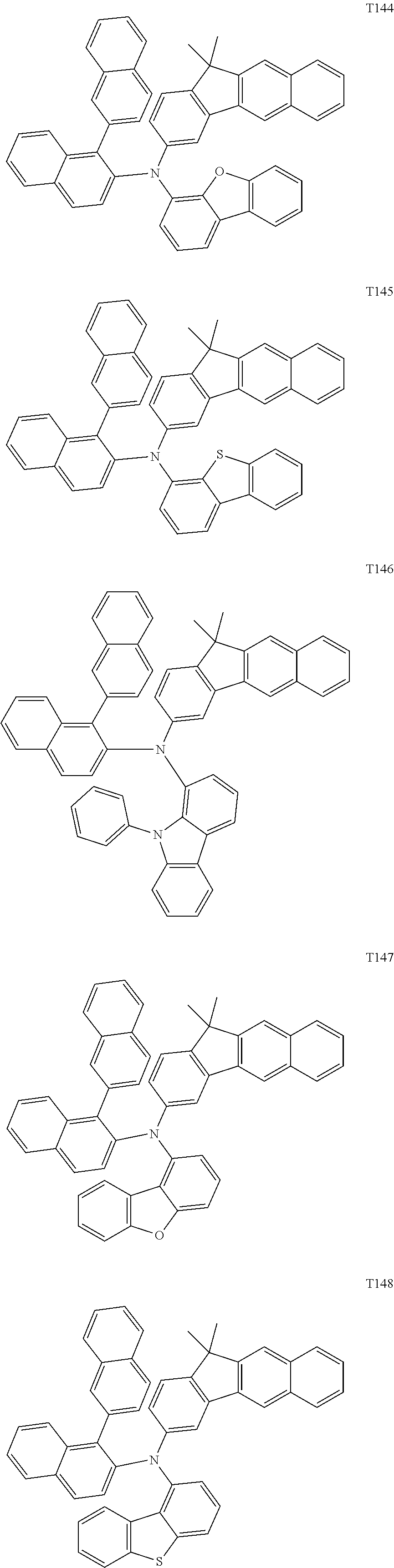 [SMILES: CC1(C)c2ccc(N(c3ccc4ccccc4c3-c3ccc4ccccc4c3)c3cccc4c3oc3ccccc34)cc2-c2cc3ccccc3cc21 ; CC1(C)c2ccc(N(c3ccc4ccccc4c3-c3ccc4ccccc4c3)c3cccc4c3sc3ccccc34)cc2-c2cc3ccccc3cc21 ; CC1(C)c2ccc(N(c3ccc4ccccc4c3-c3ccc4ccccc4c3)c3cccc4c5ccccc5n(-c5ccccc5)c34)cc2-c2cc3ccccc3cc21 ; CC1(C)c2ccc(N(c3ccc4ccccc4c3-c3ccc4ccccc4c3)c3cccc4oc5ccccc5c34)cc2-c2cc3ccccc3cc21 ; CC1(C)c2ccc(N(c3ccc4ccccc4c3-c3ccc4ccccc4c3)c3cccc4sc5ccccc5c34)cc2-c2cc3ccccc3cc21]

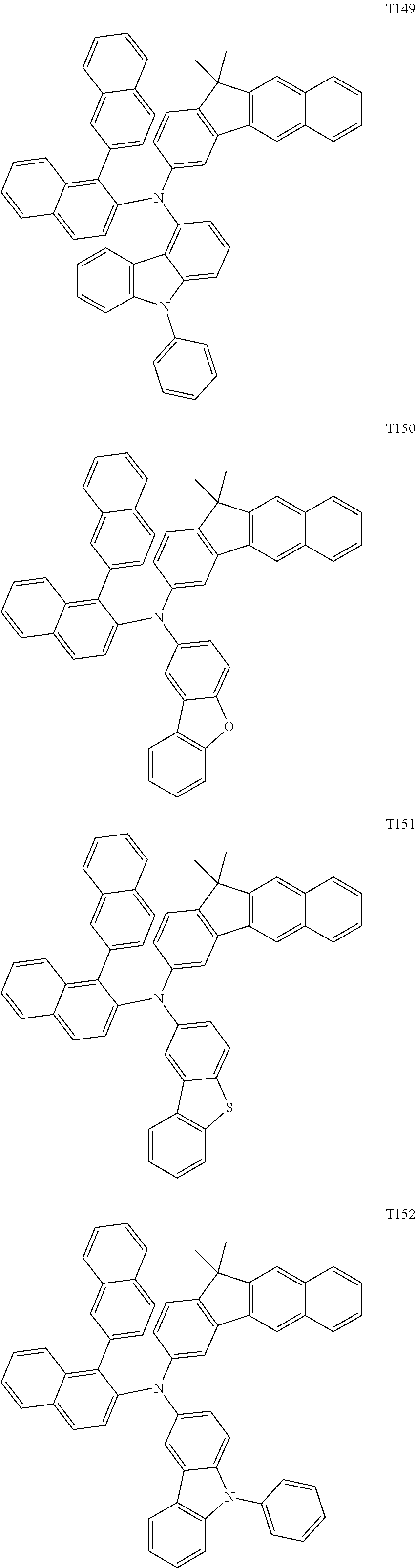 [SMILES: CC1(C)c2ccc(N(c3ccc4ccccc4c3-c3ccc4ccccc4c3)c3cccc4c3c3ccccc3n4-c3ccccc3)cc2-c2cc3ccccc3cc21 ; CC1(C)c2ccc(N(c3ccc4oc5ccccc5c4c3)c3ccc4ccccc4c3-c3ccc4ccccc4c3)cc2-c2cc3ccccc3cc21 ; CC1(C)c2ccc(N(c3ccc4sc5ccccc5c4c3)c3ccc4ccccc4c3-c3ccc4ccccc4c3)cc2-c2cc3ccccc3cc21 ; CC1(C)c2ccc(N(c3ccc4c(c3)c3ccccc3n4-c3ccccc3)c3ccc4ccccc4c3-c3ccc4ccccc4c3)cc2-c2cc3ccccc3cc21]

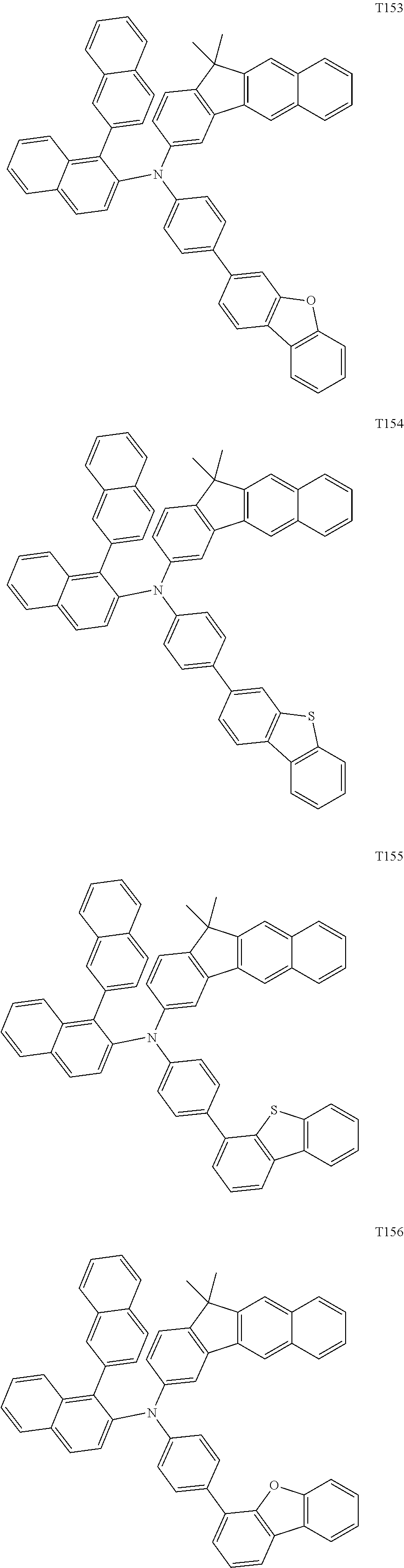 [SMILES: CC1(C)c2ccc(N(c3ccc(-c4ccc5c(c4)oc4ccccc45)cc3)c3ccc4ccccc4c3-c3ccc4ccccc4c3)cc2-c2cc3ccccc3cc21 ; CC1(C)c2ccc(N(c3ccc(-c4ccc5c(c4)sc4ccccc45)cc3)c3ccc4ccccc4c3-c3ccc4ccccc4c3)cc2-c2cc3ccccc3cc21 ; CC1(C)c2ccc(N(c3ccc(-c4cccc5c4sc4ccccc45)cc3)c3ccc4ccccc4c3-c3ccc4ccccc4c3)cc2-c2cc3ccccc3cc21 ; CC1(C)c2ccc(N(c3ccc(-c4cccc5c4oc4ccccc45)cc3)c3ccc4ccccc4c3-c3ccc4ccccc4c3)cc2-c2cc3ccccc3cc21]

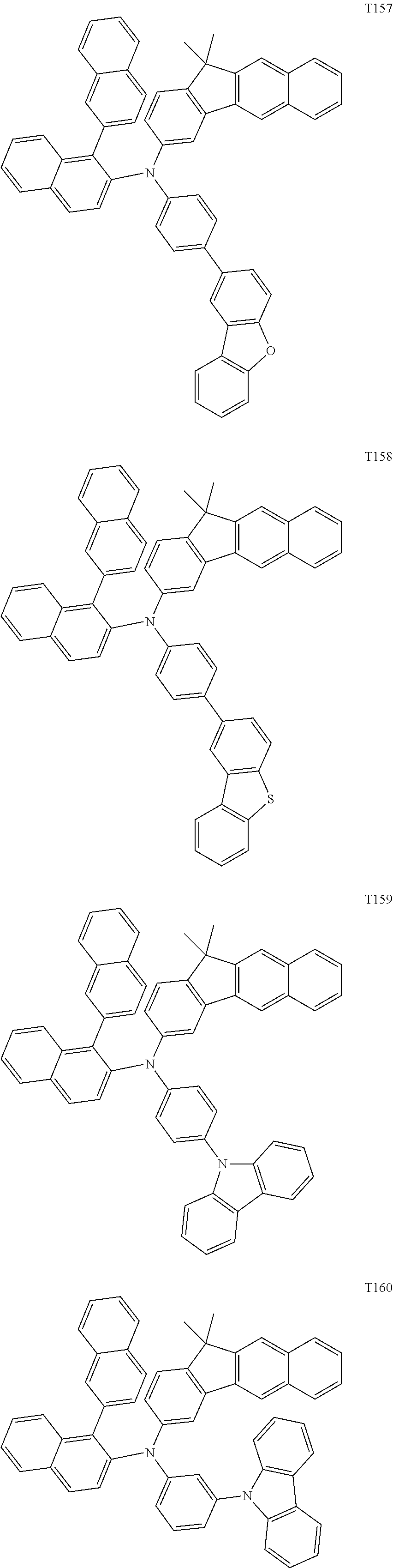 [SMILES: CC1(C)c2ccc(N(c3ccc(-c4ccc5oc6ccccc6c5c4)cc3)c3ccc4ccccc4c3-c3ccc4ccccc4c3)cc2-c2cc3ccccc3cc21 ; CC1(C)c2ccc(N(c3ccc(-c4ccc5sc6ccccc6c5c4)cc3)c3ccc4ccccc4c3-c3ccc4ccccc4c3)cc2-c2cc3ccccc3cc21 ; CC1(C)c2ccc(N(c3ccc(-n4c5ccccc5c5ccccc54)cc3)c3ccc4ccccc4c3-c3ccc4ccccc4c3)cc2-c2cc3ccccc3cc21 ; CC1(C)c2ccc(N(c3cccc(-n4c5ccccc5c5ccccc54)c3)c3ccc4ccccc4c3-c3ccc4ccccc4c3)cc2-c2cc3ccccc3cc21]

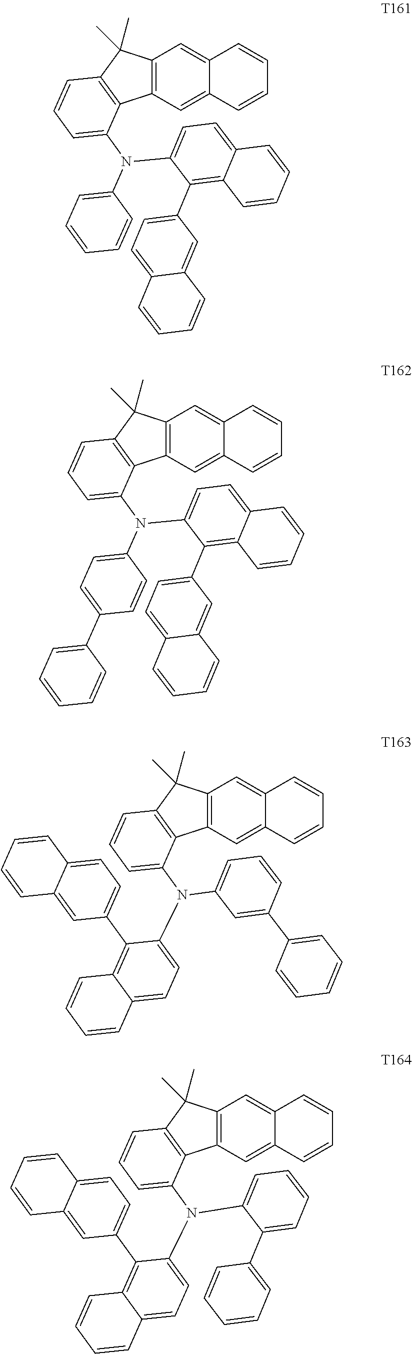 [SMILES: CC1(C)c2cc3ccccc3cc2-c2c(N(c3ccccc3)c3ccc4ccccc4c3-c3ccc4ccccc4c3)cccc21 ; CC1(C)c2cc3ccccc3cc2-c2c(N(c3ccc(-c4ccccc4)cc3)c3ccc4ccccc4c3-c3ccc4ccccc4c3)cccc21 ; CC1(C)c2cc3ccccc3cc2-c2c(N(c3cccc(-c4ccccc4)c3)c3ccc4ccccc4c3-c3ccc4ccccc4c3)cccc21 ; CC1(C)c2cc3ccccc3cc2-c2c(N(c3ccccc3-c3ccccc3)c3ccc4ccccc4c3-c3ccc4ccccc4c3)cccc21]

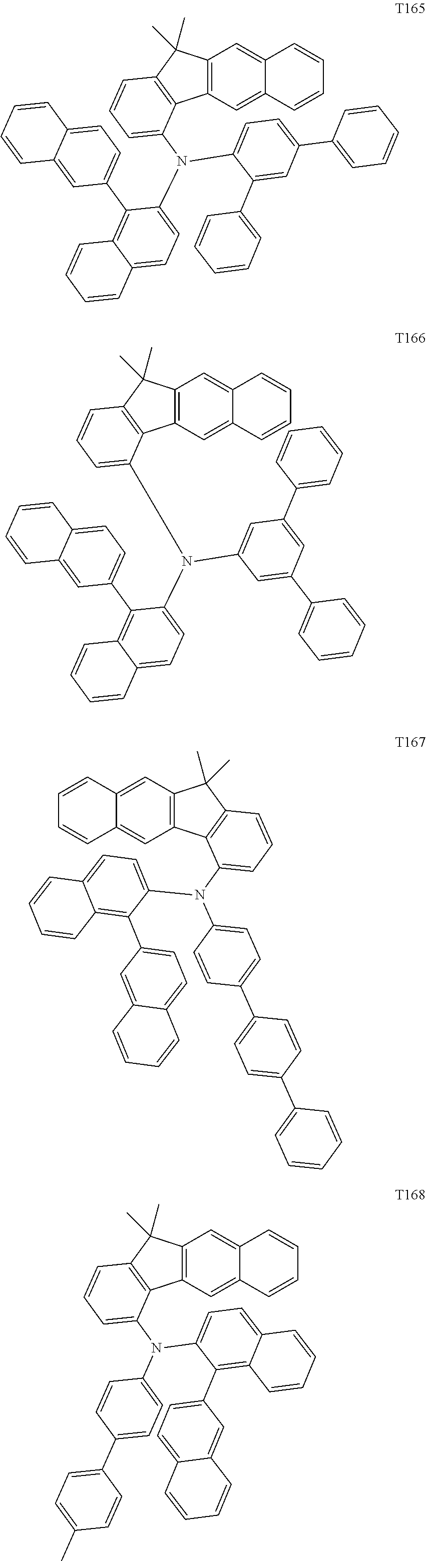 [SMILES: CC1(C)c2cc3ccccc3cc2-c2c(N(c3ccc(-c4ccccc4)cc3-c3ccccc3)c3ccc4ccccc4c3-c3ccc4ccccc4c3)cccc21 ; CC1(C)c2cc3ccccc3cc2-c2c(N(c3cc(-c4ccccc4)cc(-c4ccccc4)c3)c3ccc4ccccc4c3-c3ccc4ccccc4c3)cccc21 ; CC1(C)c2cc3ccccc3cc2-c2c(N(c3ccc(-c4ccc(-c5ccccc5)cc4)cc3)c3ccc4ccccc4c3-c3ccc4ccccc4c3)cccc21 ; Cc1ccc(-c2ccc(N(c3cccc4c3-c3cc5ccccc5cc3C4(C)C)c3ccc4ccccc4c3-c3ccc4ccccc4c3)cc2)cc1]

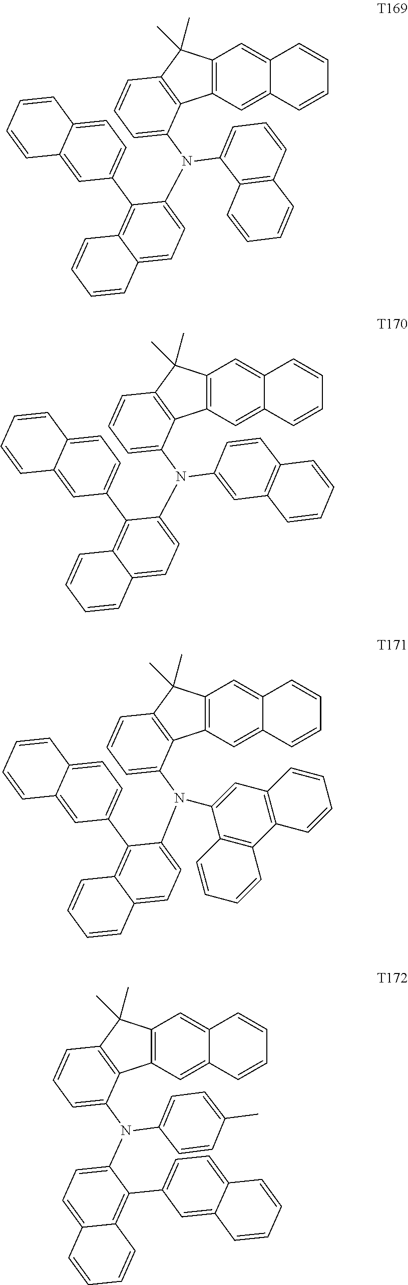 [SMILES: CC1(C)c2cc3ccccc3cc2-c2c(N(c3ccc4ccccc4c3-c3ccc4ccccc4c3)c3cccc4ccccc34)cccc21 ; CC1(C)c2cc3ccccc3cc2-c2c(N(c3ccc4ccccc4c3)c3ccc4ccccc4c3-c3ccc4ccccc4c3)cccc21 ; CC1(C)c2cc3ccccc3cc2-c2c(N(c3ccc4ccccc4c3-c3ccc4ccccc4c3)c3cc4ccccc4c4ccccc34)cccc21 ; Cc1ccc(N(c2cccc3c2-c2cc4ccccc4cc2C3(C)C)c2ccc3ccccc3c2-c2ccc3ccccc3c2)cc1]

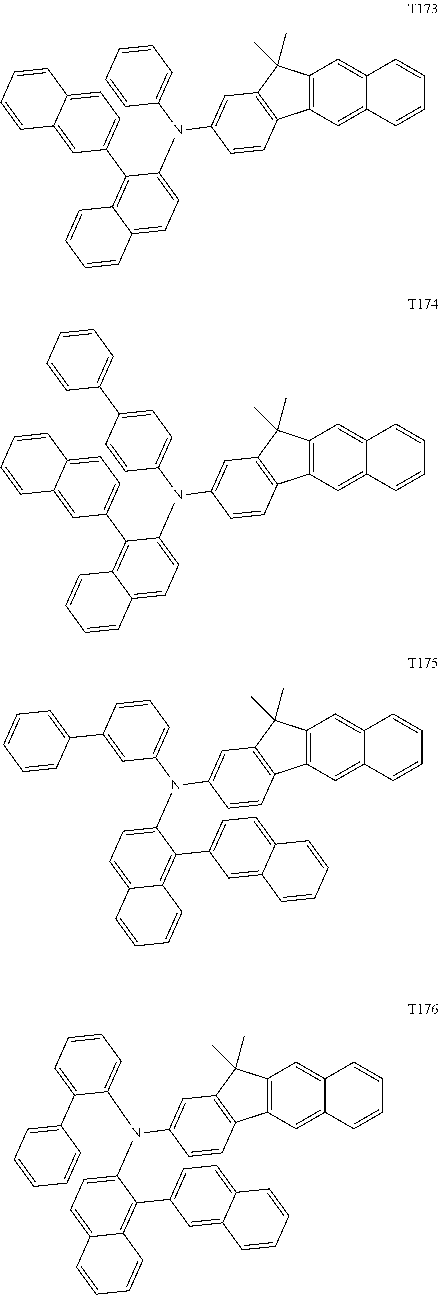 [SMILES: CC1(C)c2cc(N(c3ccccc3)c3ccc4ccccc4c3-c3ccc4ccccc4c3)ccc2-c2cc3ccccc3cc21 ; CC1(C)c2cc(N(c3ccc(-c4ccccc4)cc3)c3ccc4ccccc4c3-c3ccc4ccccc4c3)ccc2-c2cc3ccccc3cc21 ; CC1(C)c2cc(N(c3cccc(-c4ccccc4)c3)c3ccc4ccccc4c3-c3ccc4ccccc4c3)ccc2-c2cc3ccccc3cc21 ; CC1(C)c2cc(N(c3ccccc3-c3ccccc3)c3ccc4ccccc4c3-c3ccc4ccccc4c3)ccc2-c2cc3ccccc3cc21]

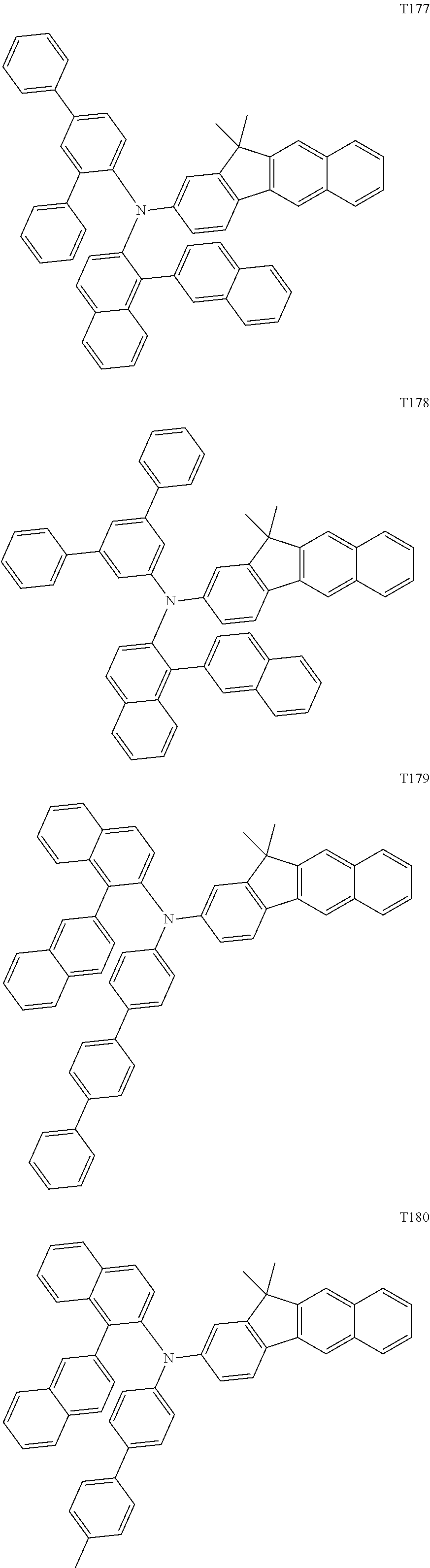 [SMILES: CC1(C)c2cc(N(c3ccc(-c4ccccc4)cc3-c3ccccc3)c3ccc4ccccc4c3-c3ccc4ccccc4c3)ccc2-c2cc3ccccc3cc21 ; CC1(C)c2cc(N(c3cc(-c4ccccc4)cc(-c4ccccc4)c3)c3ccc4ccccc4c3-c3ccc4ccccc4c3)ccc2-c2cc3ccccc3cc21 ; CC1(C)c2cc(N(c3ccc(-c4ccc(-c5ccccc5)cc4)cc3)c3ccc4ccccc4c3-c3ccc4ccccc4c3)ccc2-c2cc3ccccc3cc21 ; Cc1ccc(-c2ccc(N(c3ccc4c(c3)C(C)(C)c3cc5ccccc5cc3-4)c3ccc4ccccc4c3-c3ccc4ccccc4c3)cc2)cc1]

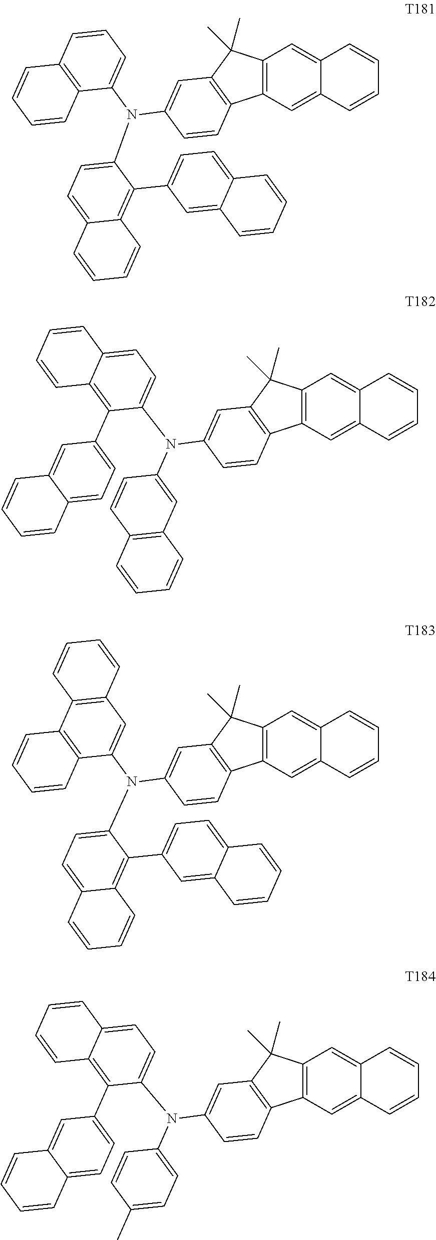 [SMILES: CC1(C)c2cc(N(c3ccc4ccccc4c3-c3ccc4ccccc4c3)c3cccc4ccccc34)ccc2-c2cc3ccccc3cc21 ; CC1(C)c2cc(N(c3ccc4ccccc4c3)c3ccc4ccccc4c3-c3ccc4ccccc4c3)ccc2-c2cc3ccccc3cc21 ; CC1(C)c2cc(N(c3ccc4ccccc4c3-c3ccc4ccccc4c3)c3cc4ccccc4c4ccccc34)ccc2-c2cc3ccccc3cc21 ; Cc1ccc(N(c2ccc3c(c2)C(C)(C)c2cc4ccccc4cc2-3)c2ccc3ccccc3c2-c2ccc3ccccc3c2)cc1]

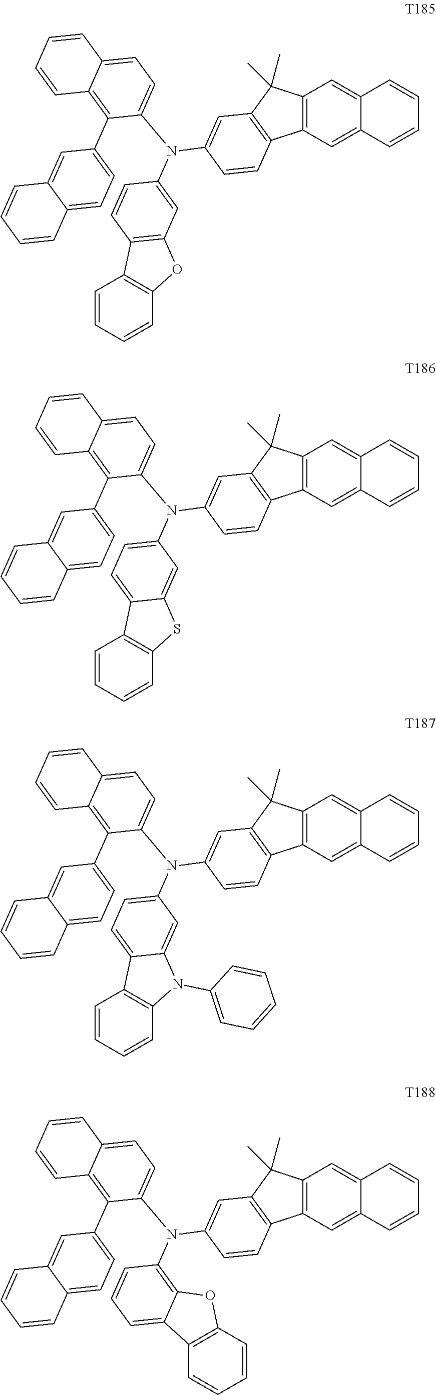 [SMILES: CC1(C)c2cc(N(c3ccc4c(c3)oc3ccccc34)c3ccc4ccccc4c3-c3ccc4ccccc4c3)ccc2-c2cc3ccccc3cc21 ; CC1(C)c2cc(N(c3ccc4c(c3)sc3ccccc34)c3ccc4ccccc4c3-c3ccc4ccccc4c3)ccc2-c2cc3ccccc3cc21 ; CC1(C)c2cc(N(c3ccc4c5ccccc5n(-c5ccccc5)c4c3)c3ccc4ccccc4c3-c3ccc4ccccc4c3)ccc2-c2cc3ccccc3cc21 ; CC1(C)c2cc(N(c3ccc4ccccc4c3-c3ccc4ccccc4c3)c3cccc4c3oc3ccccc34)ccc2-c2cc3ccccc3cc21]

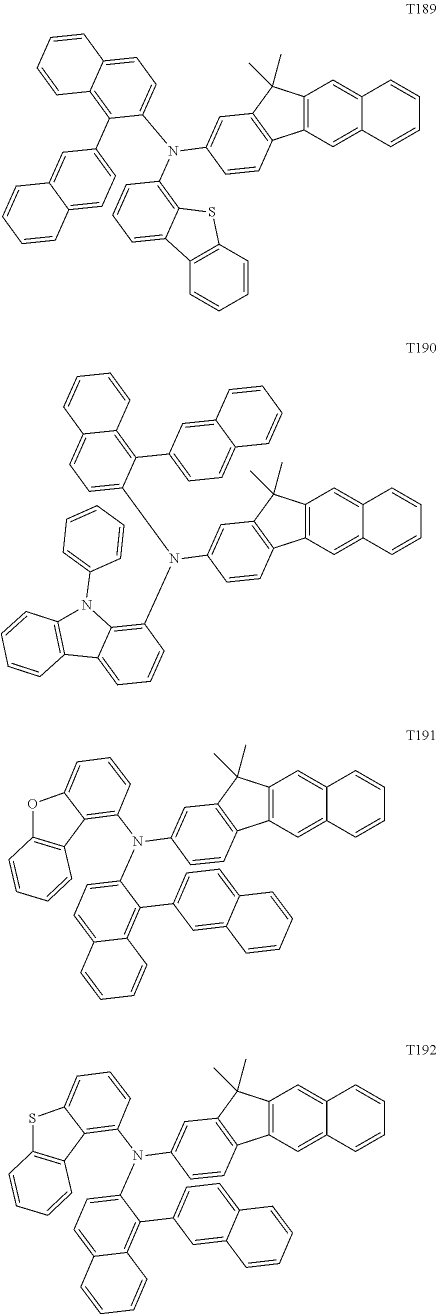 [SMILES: CC1(C)c2cc(N(c3ccc4ccccc4c3-c3ccc4ccccc4c3)c3cccc4c3sc3ccccc34)ccc2-c2cc3ccccc3cc21 ; CC1(C)c2cc(N(c3ccc4ccccc4c3-c3ccc4ccccc4c3)c3cccc4c5ccccc5n(-c5ccccc5)c34)ccc2-c2cc3ccccc3cc21 ; CC1(C)c2cc(N(c3ccc4ccccc4c3-c3ccc4ccccc4c3)c3cccc4oc5ccccc5c34)ccc2-c2cc3ccccc3cc21 ; CC1(C)c2cc(N(c3ccc4ccccc4c3-c3ccc4ccccc4c3)c3cccc4sc5ccccc5c34)ccc2-c2cc3ccccc3cc21]

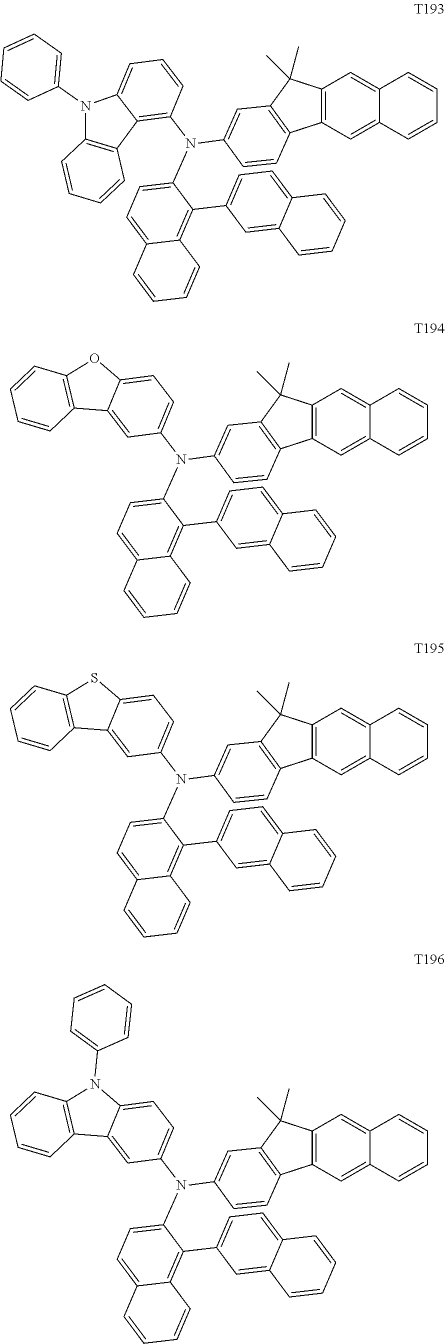 [SMILES: CC1(C)c2cc(N(c3ccc4ccccc4c3-c3ccc4ccccc4c3)c3cccc4c3c3ccccc3n4-c3ccccc3)ccc2-c2cc3ccccc3cc21 ; CC1(C)c2cc(N(c3ccc4oc5ccccc5c4c3)c3ccc4ccccc4c3-c3ccc4ccccc4c3)ccc2-c2cc3ccccc3cc21 ; CC1(C)c2cc(N(c3ccc4sc5ccccc5c4c3)c3ccc4ccccc4c3-c3ccc4ccccc4c3)ccc2-c2cc3ccccc3cc21 ; CC1(C)c2cc(N(c3ccc4c(c3)c3ccccc3n4-c3ccccc3)c3ccc4ccccc4c3-c3ccc4ccccc4c3)ccc2-c2cc3ccccc3cc21]

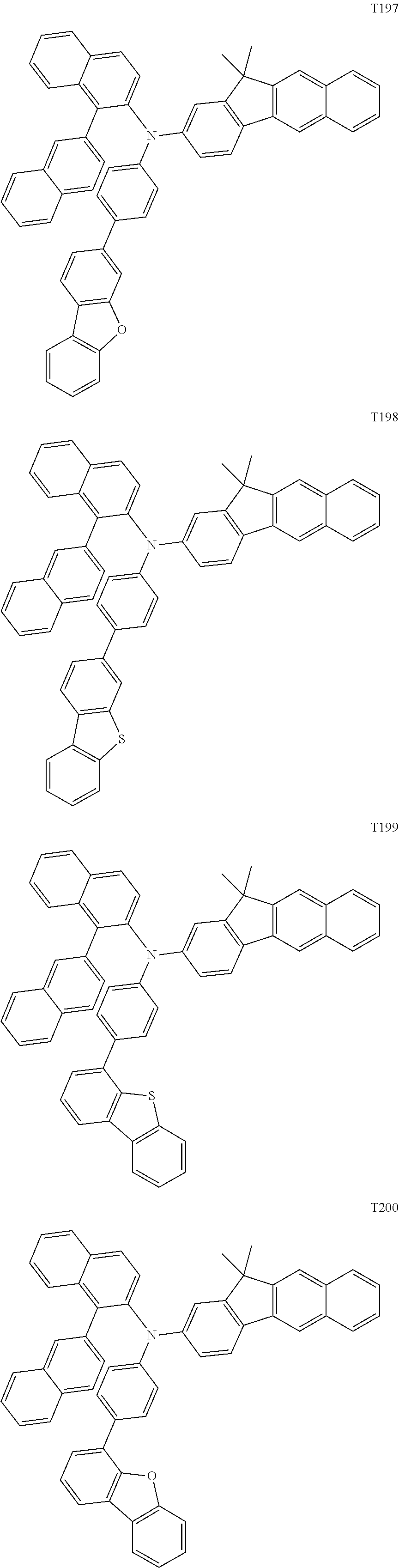 [SMILES: CC1(C)c2cc(N(c3ccc(-c4ccc5c(c4)oc4ccccc45)cc3)c3ccc4ccccc4c3-c3ccc4ccccc4c3)ccc2-c2cc3ccccc3cc21 ; CC1(C)c2cc(N(c3ccc(-c4ccc5c(c4)sc4ccccc45)cc3)c3ccc4ccccc4c3-c3ccc4ccccc4c3)ccc2-c2cc3ccccc3cc21 ; CC1(C)c2cc(N(c3ccc(-c4cccc5c4sc4ccccc45)cc3)c3ccc4ccccc4c3-c3ccc4ccccc4c3)ccc2-c2cc3ccccc3cc21 ; CC1(C)c2cc(N(c3ccc(-c4cccc5c4oc4ccccc45)cc3)c3ccc4ccccc4c3-c3ccc4ccccc4c3)ccc2-c2cc3ccccc3cc21]

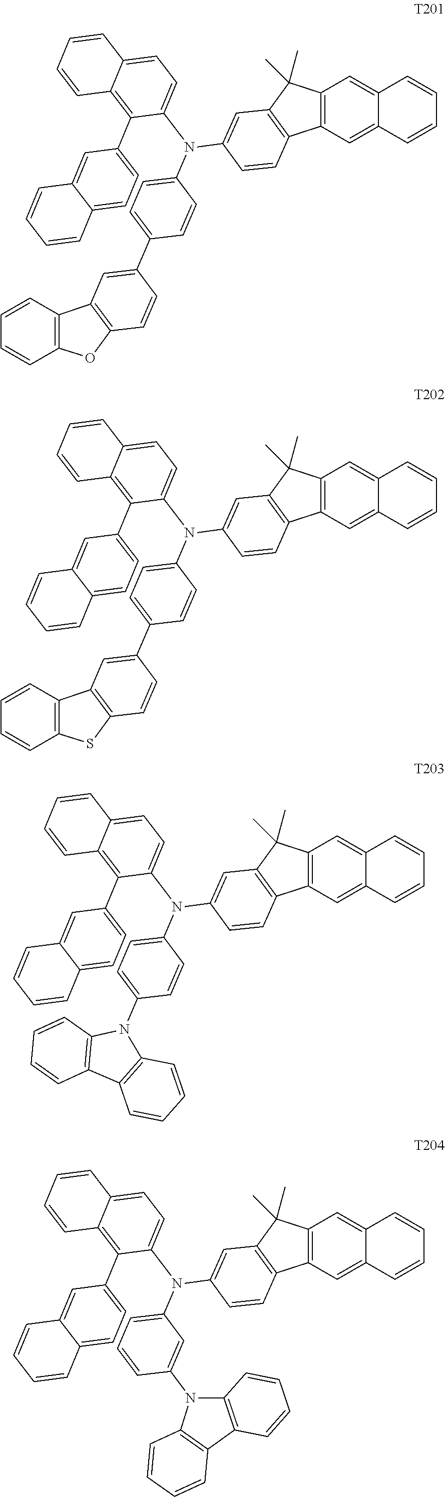 [SMILES: CC1(C)c2cc(N(c3ccc(-c4ccc5oc6ccccc6c5c4)cc3)c3ccc4ccccc4c3-c3ccc4ccccc4c3)ccc2-c2cc3ccccc3cc21 ; CC1(C)c2cc(N(c3ccc(-c4ccc5sc6ccccc6c5c4)cc3)c3ccc4ccccc4c3-c3ccc4ccccc4c3)ccc2-c2cc3ccccc3cc21 ; CC1(C)c2cc(N(c3ccc(-n4c5ccccc5c5ccccc54)cc3)c3ccc4ccccc4c3-c3ccc4ccccc4c3)ccc2-c2cc3ccccc3cc21 ; CC1(C)c2cc(N(c3cccc(-n4c5ccccc5c5ccccc54)c3)c3ccc4ccccc4c3-c3ccc4ccccc4c3)ccc2-c2cc3ccccc3cc21]

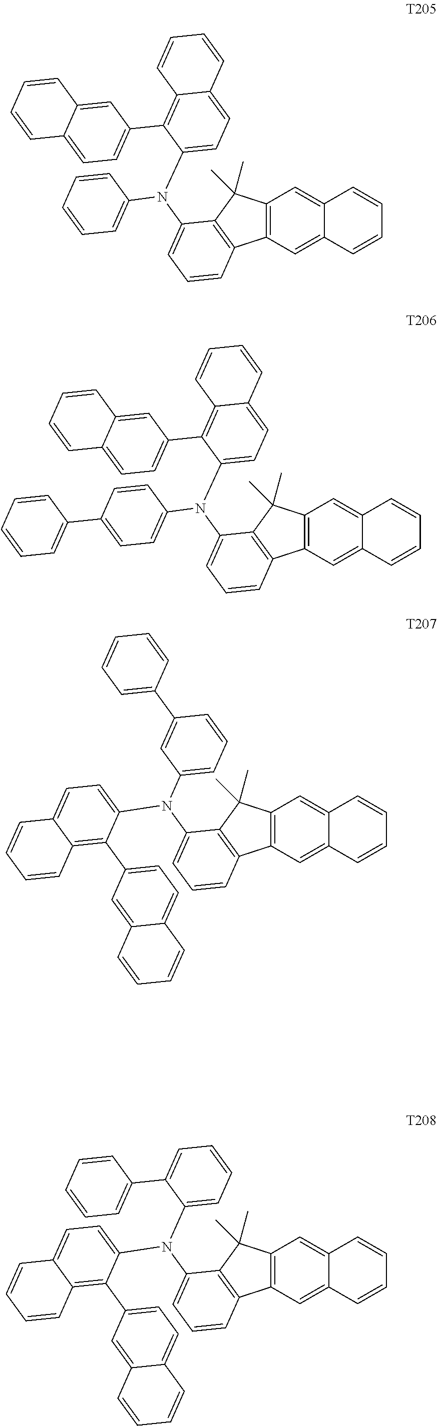 [SMILES: CC1(C)c2cc3ccccc3cc2-c2cccc(N(c3ccccc3)c3ccc4ccccc4c3-c3ccc4ccccc4c3)c21 ; CC1(C)c2cc3ccccc3cc2-c2cccc(N(c3ccc(-c4ccccc4)cc3)c3ccc4ccccc4c3-c3ccc4ccccc4c3)c21 ; CC1(C)c2cc3ccccc3cc2-c2cccc(N(c3cccc(-c4ccccc4)c3)c3ccc4ccccc4c3-c3ccc4ccccc4c3)c21 ; CC1(C)c2cc3ccccc3cc2-c2cccc(N(c3ccccc3-c3ccccc3)c3ccc4ccccc4c3-c3ccc4ccccc4c3)c21]

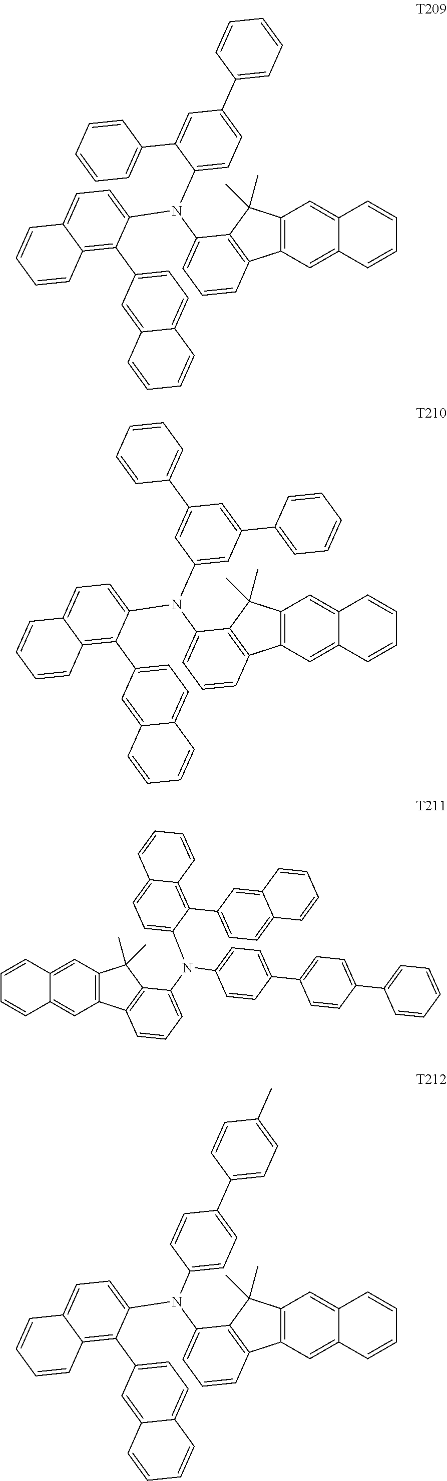

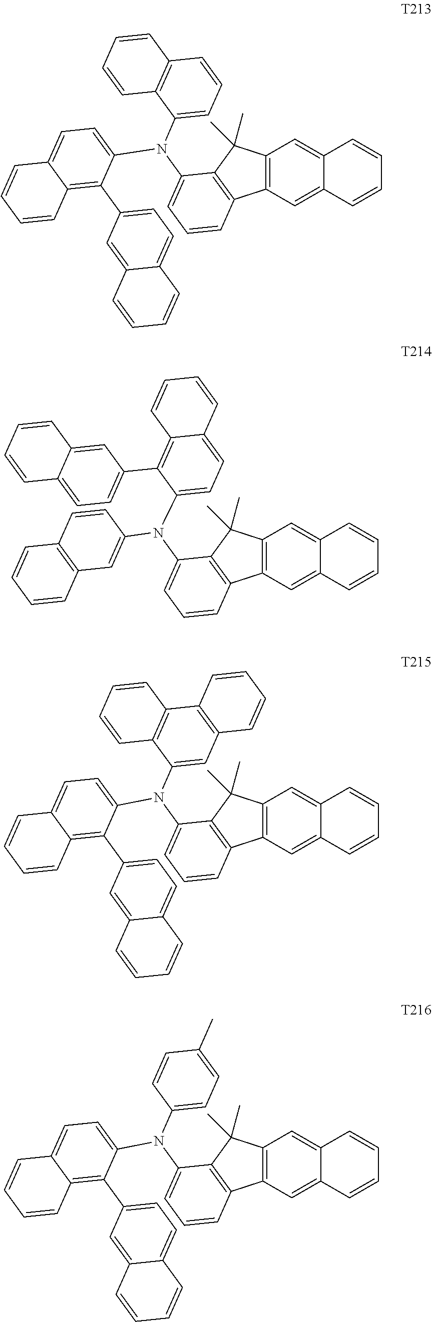 [SMILES: CC1(C)c2cc3ccccc3cc2-c2cccc(N(c3ccc4ccccc4c3-c3ccc4ccccc4c3)c3cccc4ccccc34)c21 ; CC1(C)c2cc3ccccc3cc2-c2cccc(N(c3ccc4ccccc4c3)c3ccc4ccccc4c3-c3ccc4ccccc4c3)c21 ; CC1(C)c2cc3ccccc3cc2-c2cccc(N(c3ccc4ccccc4c3-c3ccc4ccccc4c3)c3cc4ccccc4c4ccccc34)c21 ; Cc1ccc(N(c2cccc3c2C(C)(C)c2cc4ccccc4cc2-3)c2ccc3ccccc3c2-c2ccc3ccccc3c2)cc1]

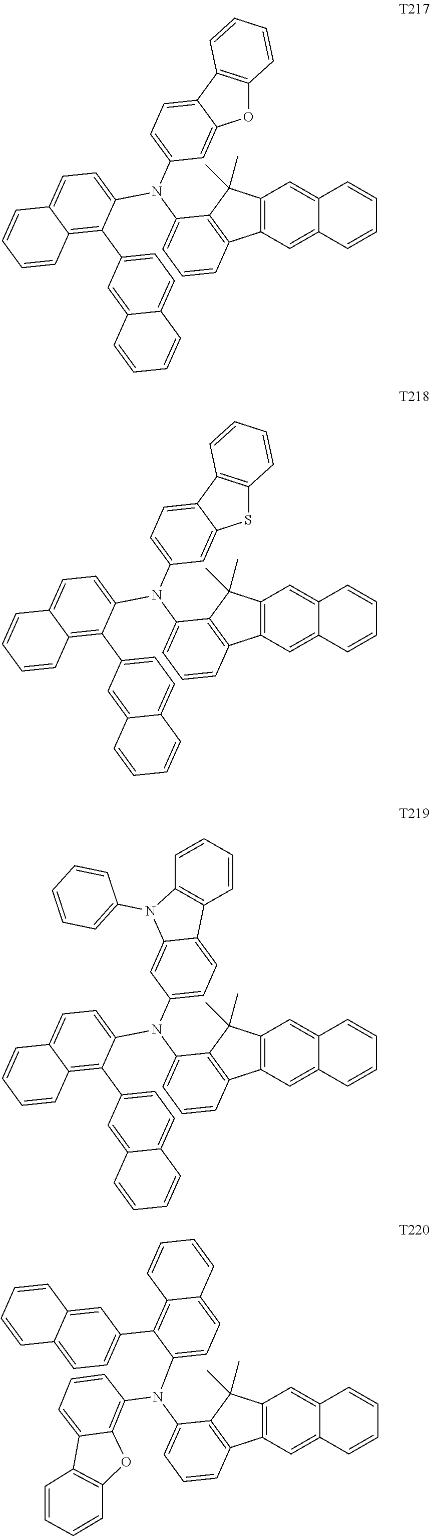

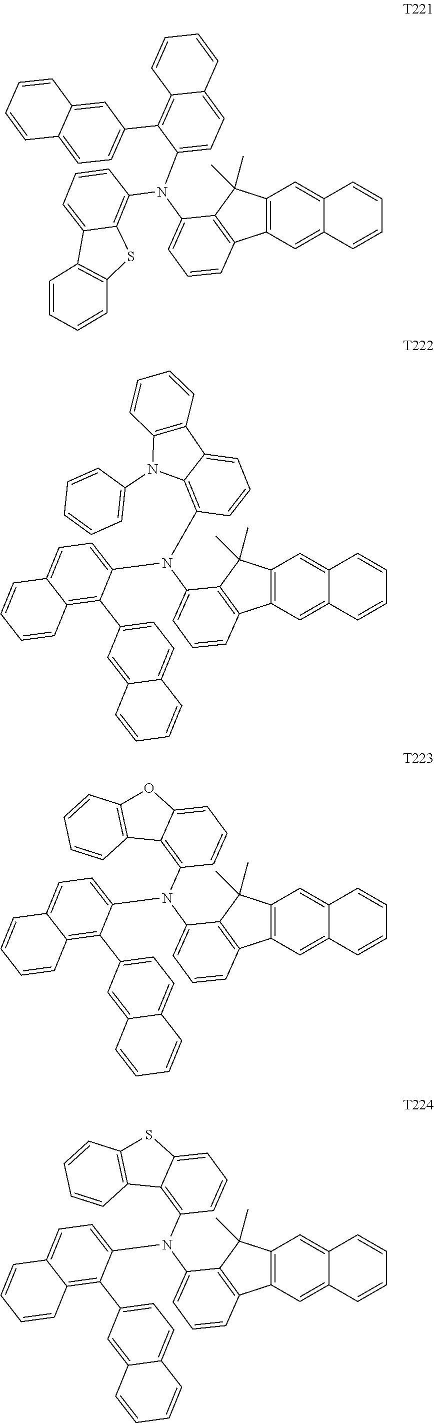 [SMILES: CC1(C)c2cc3ccccc3cc2-c2cccc(N(c3ccc4ccccc4c3-c3ccc4ccccc4c3)c3cccc4c3sc3ccccc34)c21 ; CC1(C)c2cc3ccccc3cc2-c2cccc(N(c3ccc4ccccc4c3-c3ccc4ccccc4c3)c3cccc4c5ccccc5n(-c5ccccc5)c34)c21 ; CC1(C)c2cc3ccccc3cc2-c2cccc(N(c3ccc4ccccc4c3-c3ccc4ccccc4c3)c3cccc4oc5ccccc5c34)c21 ; CC1(C)c2cc3ccccc3cc2-c2cccc(N(c3ccc4ccccc4c3-c3ccc4ccccc4c3)c3cccc4sc5ccccc5c34)c21]

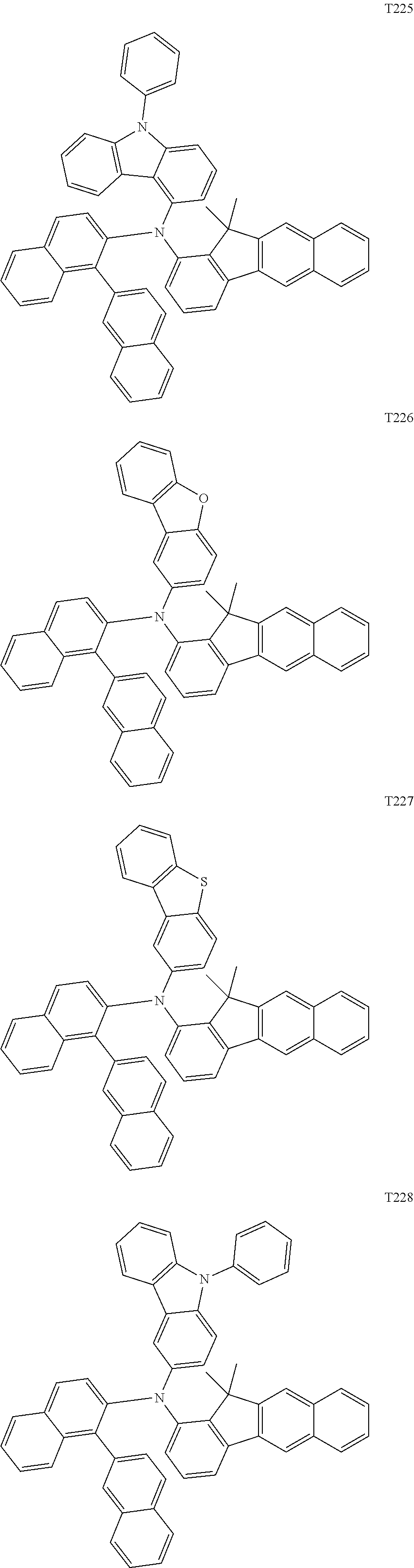

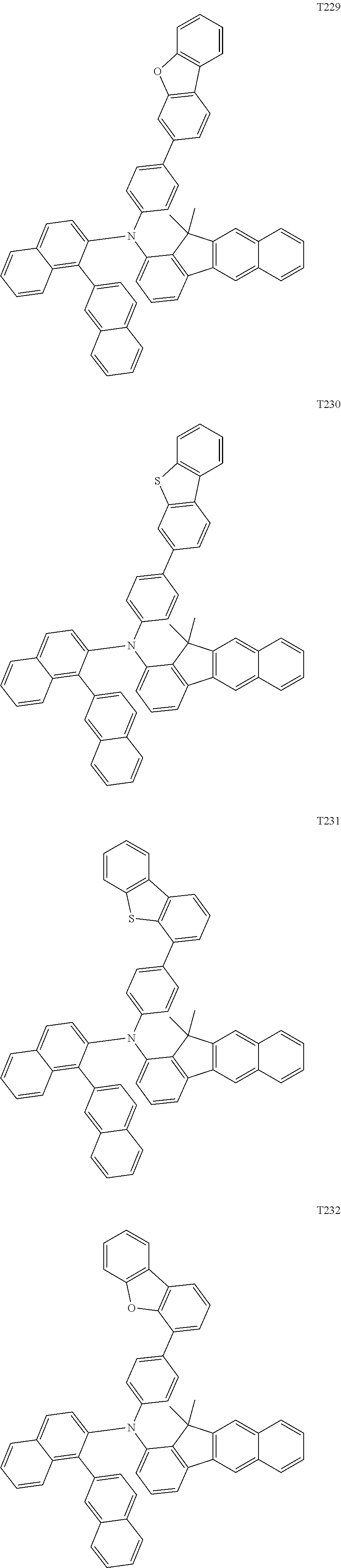 [SMILES: CC1(C)c2cc3ccccc3cc2-c2cccc(N(c3ccc(-c4ccc5c(c4)oc4ccccc45)cc3)c3ccc4ccccc4c3-c3ccc4ccccc4c3)c21 ; CC1(C)c2cc3ccccc3cc2-c2cccc(N(c3ccc(-c4ccc5c(c4)sc4ccccc45)cc3)c3ccc4ccccc4c3-c3ccc4ccccc4c3)c21 ; CC1(C)c2cc3ccccc3cc2-c2cccc(N(c3ccc(-c4cccc5c4sc4ccccc45)cc3)c3ccc4ccccc4c3-c3ccc4ccccc4c3)c21 ; CC1(C)c2cc3ccccc3cc2-c2cccc(N(c3ccc(-c4cccc5c4oc4ccccc45)cc3)c3ccc4ccccc4c3-c3ccc4ccccc4c3)c21]

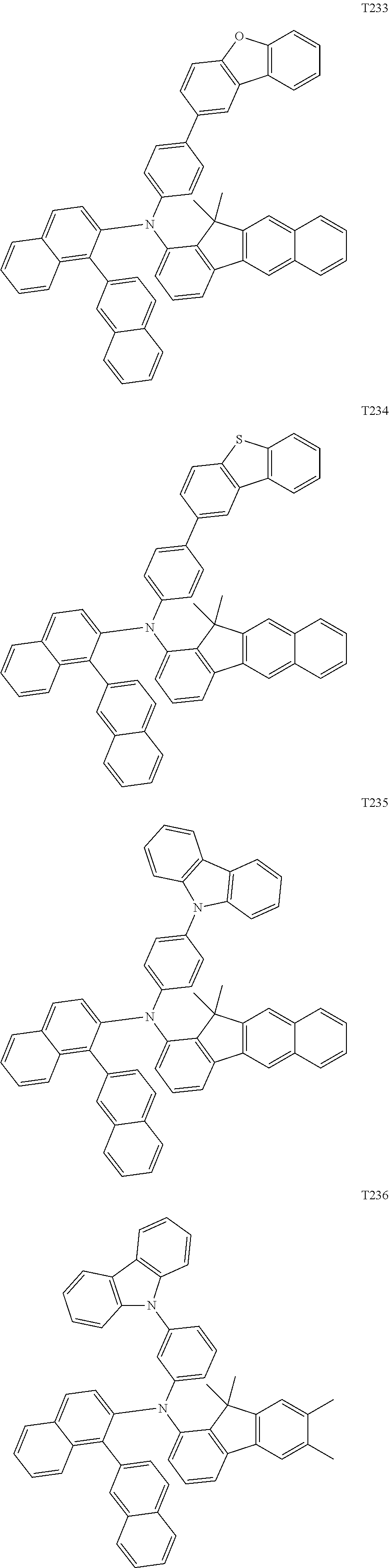 [SMILES: CC1(C)c2cc3ccccc3cc2-c2cccc(N(c3ccc(-c4ccc5oc6ccccc6c5c4)cc3)c3ccc4ccccc4c3-c3ccc4ccccc4c3)c21 ; CC1(C)c2cc3ccccc3cc2-c2cccc(N(c3ccc(-c4ccc5sc6ccccc6c5c4)cc3)c3ccc4ccccc4c3-c3ccc4ccccc4c3)c21 ; CC1(C)c2cc3ccccc3cc2-c2cccc(N(c3ccc(-n4c5ccccc5c5ccccc54)cc3)c3ccc4ccccc4c3-c3ccc4ccccc4c3)c21 ; Cc1cc2c(cc1C)C(C)(C)c1c-2cccc1N(c1cccc(-n2c3ccccc3c3ccccc32)c1)c1ccc2ccccc2c1-c1ccc2ccccc2c1]

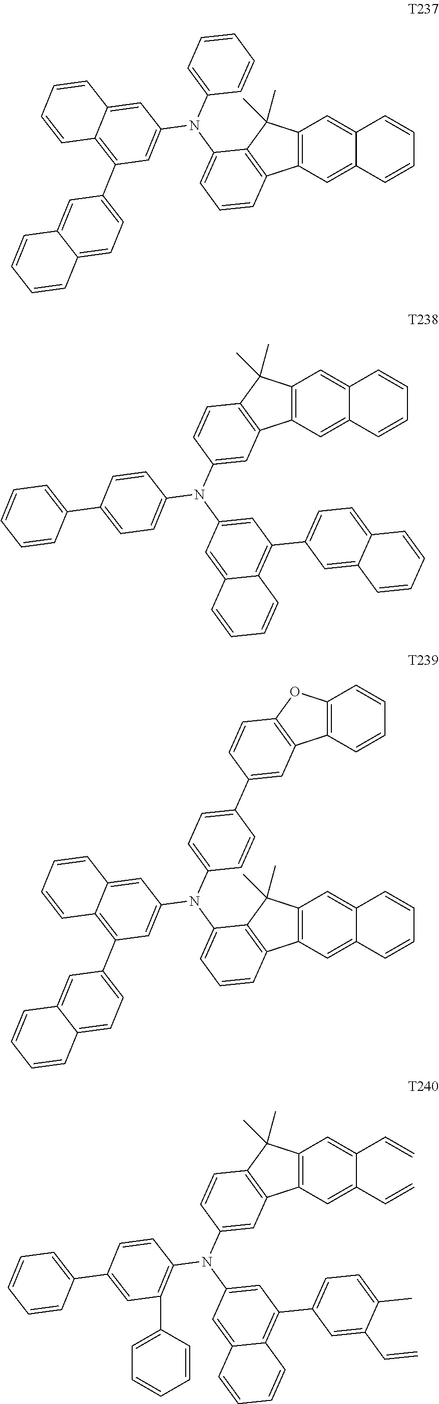 [SMILES: CC1(C)c2cc3ccccc3cc2-c2cccc(N(c3ccccc3)c3cc(-c4ccc5ccccc5c4)c4ccccc4c3)c21 ; CC1(C)c2ccc(N(c3ccc(-c4ccccc4)cc3)c3cc(-c4ccc5ccccc5c4)c4ccccc4c3)cc2-c2cc3ccccc3cc21 ; CC1(C)c2cc3ccccc3cc2-c2cccc(N(c3ccc(-c4ccc5oc6ccccc6c5c4)cc3)c3cc(-c4ccc5ccccc5c4)c4ccccc4c3)c21 ; C=Cc1cc(-c2cc(N(c3ccc4c(c3)-c3cc(C=C)c(C=C)cc3C4(C)C)c3ccc(-c4ccccc4)cc3-c3ccccc3)cc3ccccc23)ccc1C]

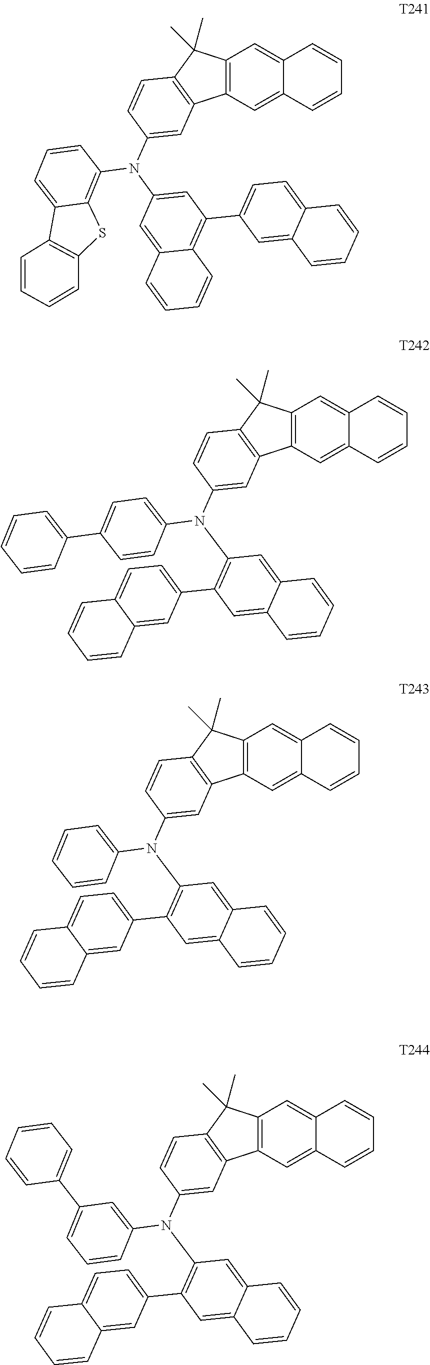 [SMILES: CC1(C)c2ccc(N(c3cc(-c4ccc5ccccc5c4)c4ccccc4c3)c3cccc4c3sc3ccccc34)cc2-c2cc3ccccc3cc21 ; CC1(C)c2ccc(N(c3ccc(-c4ccccc4)cc3)c3cc4ccccc4cc3-c3ccc4ccccc4c3)cc2-c2cc3ccccc3cc21 ; CC1(C)c2ccc(N(c3ccccc3)c3cc4ccccc4cc3-c3ccc4ccccc4c3)cc2-c2cc3ccccc3cc21 ; CC1(C)c2ccc(N(c3cccc(-c4ccccc4)c3)c3cc4ccccc4cc3-c3ccc4ccccc4c3)cc2-c2cc3ccccc3cc21]

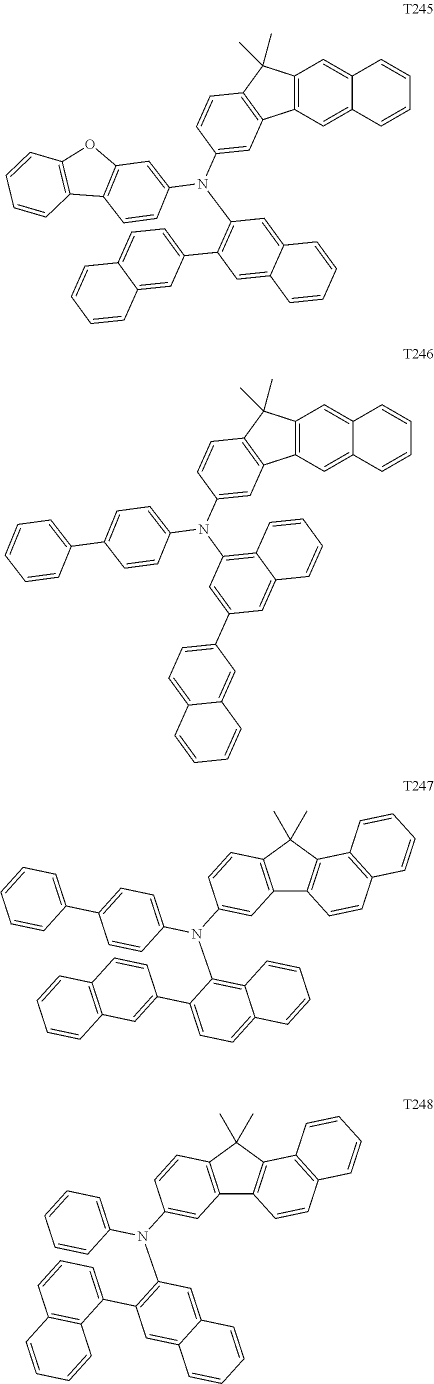 [SMILES: CC1(C)c2ccc(N(c3ccc4c(c3)oc3ccccc34)c3cc4ccccc4cc3-c3ccc4ccccc4c3)cc2-c2cc3ccccc3cc21 ; CC1(C)c2ccc(N(c3ccc(-c4ccccc4)cc3)c3cc(-c4ccc5ccccc5c4)cc4ccccc34)cc2-c2cc3ccccc3cc21 ; CC1(C)c2ccc(N(c3ccc(-c4ccccc4)cc3)c3c(-c4ccc5ccccc5c4)ccc4ccccc34)cc2-c2ccc3ccccc3c21 ; CC1(C)c2ccc(N(c3ccccc3)c3cc4ccccc4cc3-c3cccc4ccccc34)cc2-c2ccc3ccccc3c21]

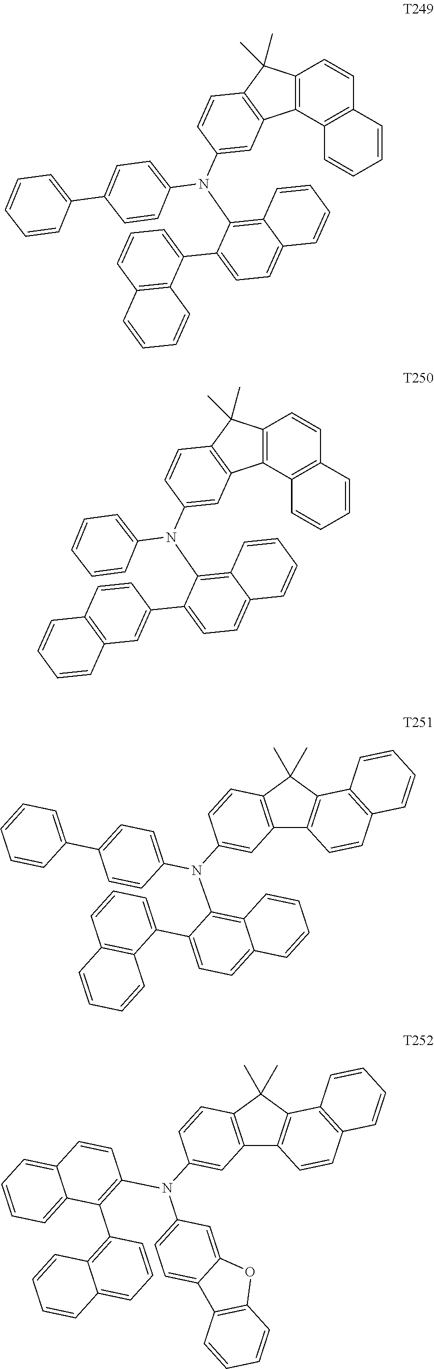 [SMILES: CC1(C)c2ccc(N(c3ccc(-c4ccccc4)cc3)c3c(-c4cccc5ccccc45)ccc4ccccc34)cc2-c2c1ccc1ccccc21 ; CC1(C)c2ccc(N(c3ccccc3)c3c(-c4ccc5ccccc5c4)ccc4ccccc34)cc2-c2c1ccc1ccccc21 ; CC1(C)c2ccc(N(c3ccc(-c4ccccc4)cc3)c3c(-c4cccc5ccccc45)ccc4ccccc34)cc2-c2ccc3ccccc3c21 ; CC1(C)c2ccc(N(c3ccc4c(c3)oc3ccccc34)c3ccc4ccccc4c3-c3cccc4ccccc34)cc2-c2ccc3ccccc3c21]

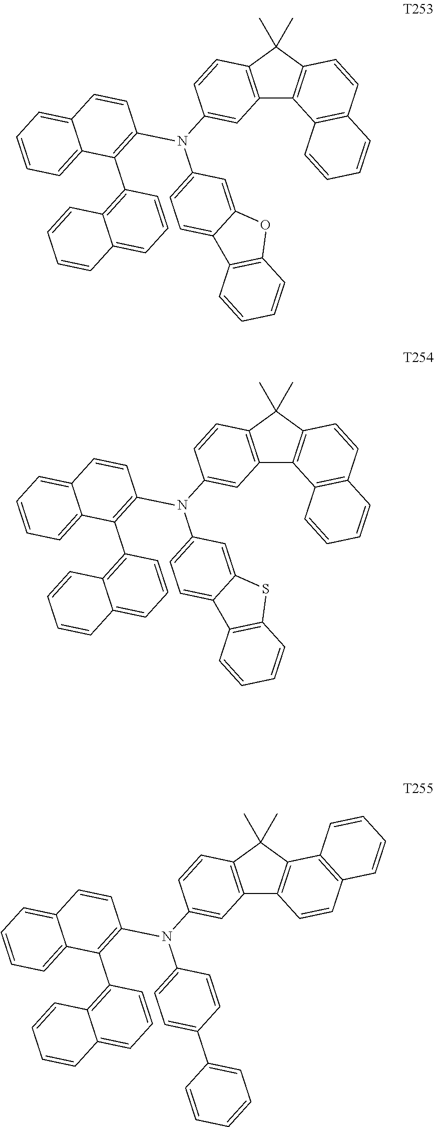 [SMILES: CC1(C)c2ccc(N(c3ccc4c(c3)oc3ccccc34)c3ccc4ccccc4c3-c3cccc4ccccc34)cc2-c2c1ccc1ccccc21 ; CC1(C)c2ccc(N(c3ccc4c(c3)sc3ccccc34)c3ccc4ccccc4c3-c3cccc4ccccc34)cc2-c2c1ccc1ccccc21 ; CC1(C)c2ccc(N(c3ccc(-c4ccccc4)cc3)c3ccc4ccccc4c3-c3cccc4ccccc34)cc2-c2ccc3ccccc3c21]

The present invention provides an application of the above compound in an organic electroluminescent device. The above compound is preferably used as an electron blocking layer of an organic electroluminescent device.

The present invention provides an organic electroluminescent device, including a substrate, a first electrode, a second electrode, and at least one organic layer located between the first electrode and the second organic compounds, and the organic layer contains at least one of the above compound. The organic layer preferably includes an electron blocking layer, and the electron blocking layer contains the above compound.

### Beneficial Effects of the Invention

In this present invention, a “naphthalene-triaryl amine” structure is designed to effectively regulate and control the triplet-state energy level of target molecules, thus obtaining a novel hole-transport material with good hole transport performance and high triplet-state energy level. Specifically, the “naphthalene-triaryl amine” is designed as a structure where there is a substituted or unsubstituted C₆-C₃₀ aryl or substituted or unsubstituted C₃-C₃₀ heteroaryl in an ortho position of diarylamido on the naphthalene ring; or, the “naphthalene-triaryl amine” is designed as a structure where one aryl in the “tri-“aryl” amine” is binaphthylyl; the other aryl is substituted or unsubstituted benzodimethyl fluorenyl, and the third aryl is substituted or unsubstituted C₆-C₃₀ aryl or substituted or unsubstituted C₃-C₃₀ heteroaryl. In this way, the triplet-state energy level of molecules may be up-regulated to obtain a novel hole-transport material with good hole transport performance.

In case that there is the above specific substituent in the ortho position of diarylamido on the naphthalene ring, a cycloalkyl group is further brought into a specific site of molecules to promote the arrangement of molecules in a spreading way, which improves the optical extraction efficiency while promoting the carrier transmission performance, thus promoting the photoelectric and life performance of the device. In this way, the material may be used as the hole transport layer material or electron blocking layer in the organic electroluminescent device to improve the luminous efficiency, reduce starting voltage and prolong service life of the device. If another naphthalene ring is linked in the 1-position of the naphthalene ring of the molecule, and diarylamine is linked onto the 2-position, the compound of the present invention has a large plane structure π, which may effectively change the molecular space structure and facilitate the improvement of molecule accumulation within the film. Further, the ortho position substitution limits the rotation of an aromatic ring on N atoms, which enhances the stability of the material. In this way, the compound is used as a hole transport layer material and/or an electron blocking layer of an organic electroluminescent device, which may improve the luminous efficiency, reduce starting voltage and prolong service life of the device.

In case that arylamine contains binaphthylyl, benzofluorenyl and a specific aromatic group linked to N, the compound has good hole injection and hole transport performance, good refraction coefficient and higher phase-transition temperature. Therefore, the above compound used in an OLED device may improve the luminous efficiency of the device, reduce low driving voltage and prolong service life.

### BRIEF DESCRIPTION OF THE DRAWINGS

FIG. 1 is a diagram showing the molecular structure model of the compound N1 of the present invention.

FIG. 2 is a diagram showing the molecular structure model of the compound N191 of the present invention.

FIG. 3 is a diagram showing the molecular structure model of the compound EMT-3 of the present invention.

FIG. 4 is a diagram showing the molecular structure model of the compound EMT-4 of the present invention.

### DETAILED DESCRIPTION OF THE EMBODIMENTS

The technical solution of the present invention will be further described by reference to the following detailed embodiments. A person skilled in the art should know that the embodiments are merely used to help understanding the present invention but are not construed as limiting the scope of the invention.

### Composition of the Organic Electroluminescent of the Present Invention

In a detailed embodiment, a substrate may be used below a first electrode or above a second electrode. The substrate is made of a glass or polymer material with excellent mechanical strength, heat stability, waterproofness and transparency. Moreover, the substrate as a display may be also provided with a thin film transistor (TFT).

The first electrode may be formed by a way of sputtering or depositing a material to be used as the first electrode on the substrate. The first electrode may be made of indium tin oxide (ITO), indium zinc oxide (IZO), SnO₂, ZnO and other oxides, namely, transparent conductive materials and any combination thereof when the first electrode serves as an anode. The first electrode may be made of Mg, Ag, Al, Al—Li, Ca, Mg—In, Mg—Ag, and other metals or alloys and any combination thereof when the first electrode serves as a cathode.

The organic layer may be formed onto the electrodes by vacuum thermal evaporation, rotary coating, printing and other methods. The compound used as the organic layer may be organic small organic molecules, organic macromolecules and polymers, and combinations thereof.

The hole transport region is located between the anode and the luminescent layer. The hole transport region may be a hole transport layer (HTL) with a single-layer structure, including a single-layer HTL only containing a compound and a single-layer HTL containing a plurality of compounds. The hole transport region also may be a multilayered structure including at least one of a hole injection layer (HIL), a hole transport layer (HTL) and an electron blocking layer (EBL).

In one aspect of the present invention, the electron blocking layer in the hole transport region may be selected from one or more of compounds of the present invention. At this time, HTL in the hole transport region may be selected from, but not limited to, phthalocyanine derivatives, e.g., CuPc, conductive polymers or polymers containing conductive dopants, such as, polyhenylene vinylene, polyaniline/dodecylbenzene sulfonic acid (Pam/DBSA), poly(3,4-ethylenedioxothiophene)/poly(4-styrene sulfonate) (PEDOT/PSS), polyaniline/camphorsulfonic acid (Pani/CSA), polyaniline/poly(4-styrene sulfonate) (Pani/PSS), aromatic amine derivatives, such as, compounds as shown in the following HT-1 to HT-34, or any combination thereof.

In another aspect of the present invention, the HTL in the hole transport region may be selected from one or more of compounds of the present invention. At this time, the electron blocking layer in the hole transport region may be selected from, but not limited to, phthalocyanine derivatives, e.g., CuPc, conductive polymers or polymers containing conductive dopants, such as, polyhenylene vinylene, polyaniline/dodecylbenzene sulfonic acid (Pam/DBSA), poly(3,4-ethylenedioxothiopheneypoly(4-styrene sulfonate) (PEDOT/PSS), polyaniline/camphorsulfonic acid (Pani/CSA), polyaniline/poly(4-styrene sulfonate) (Pani/PSS), aromatic amine derivatives, such as, compounds as shown in the following HT-1 to HT-34, or any combination thereof.

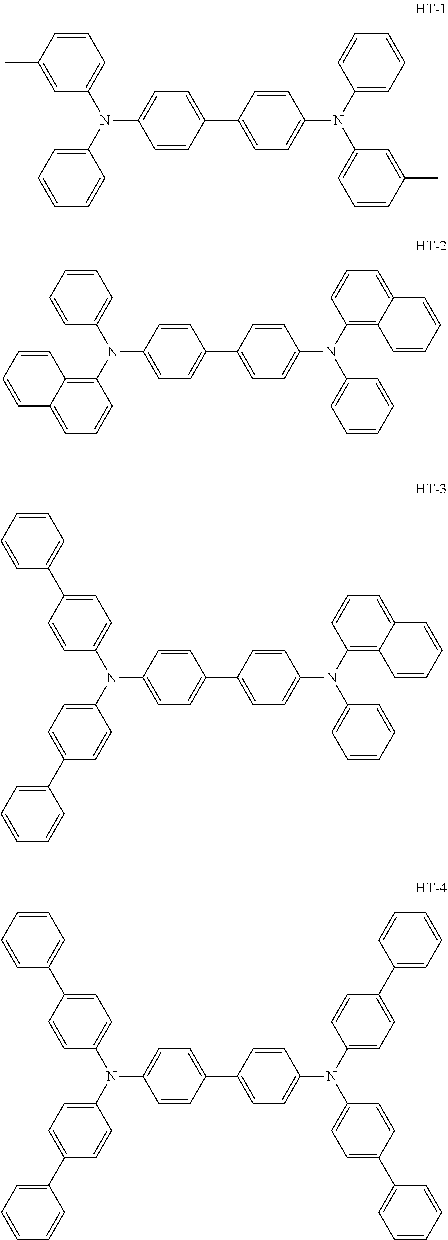

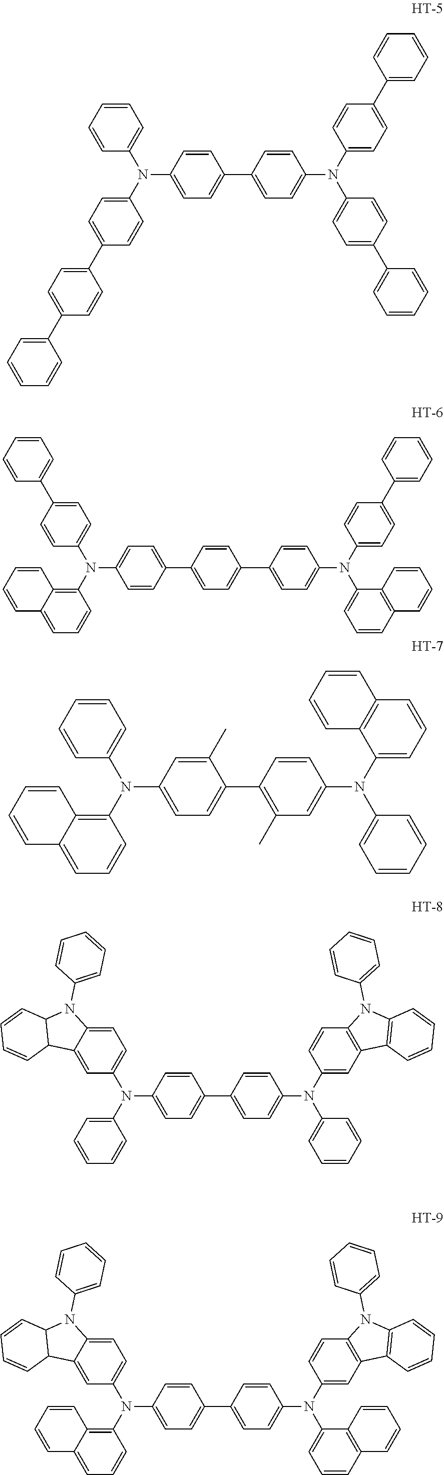

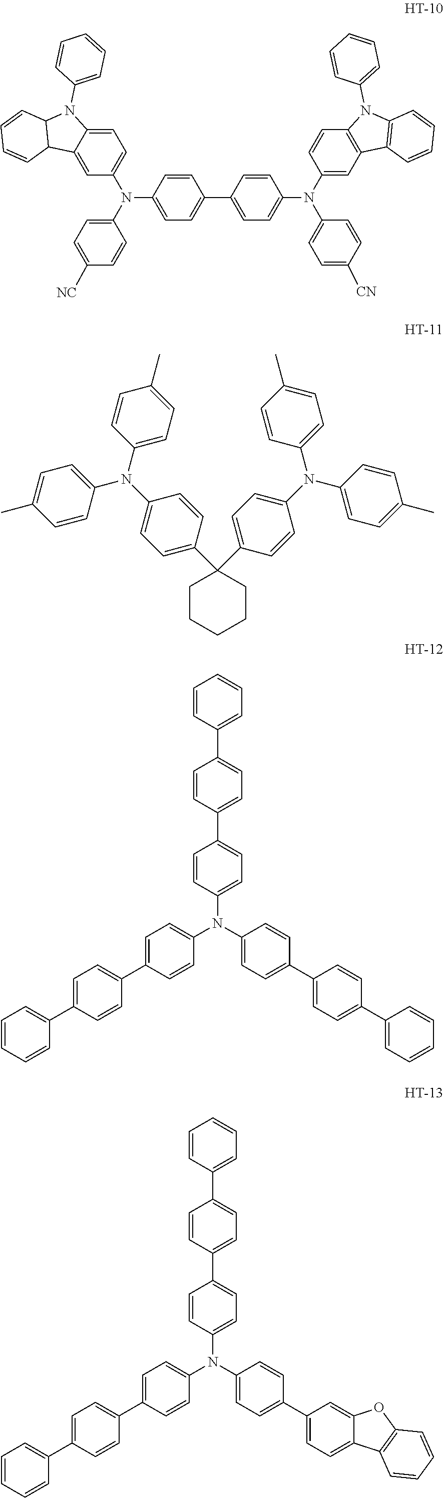

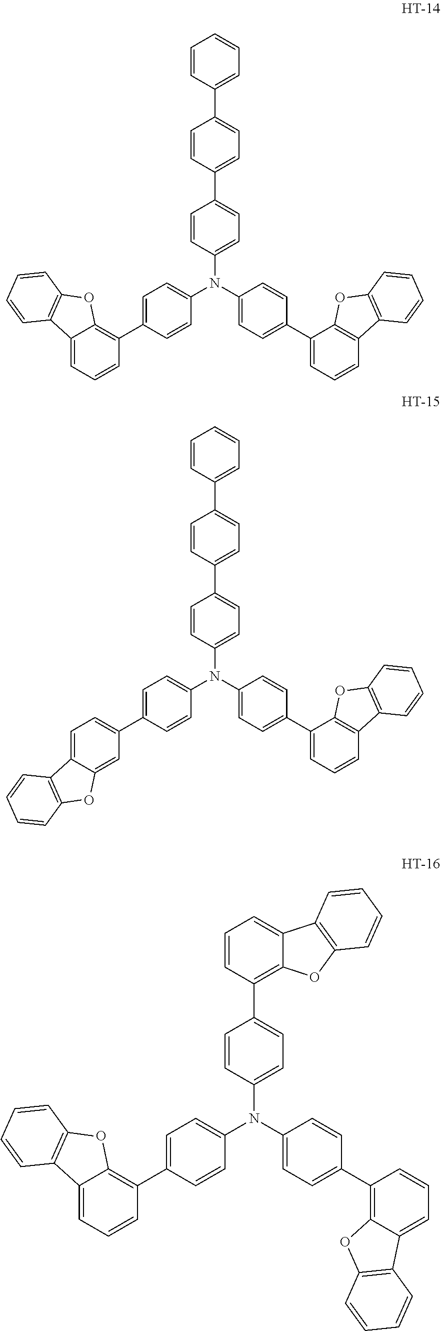 [SMILES: c1ccc(-c2ccc(-c3ccc(N(c4ccc(-c5cccc6c5oc5ccccc56)cc4)c4ccc(-c5cccc6c5oc5ccccc56)cc4)cc3)cc2)cc1 ; c1ccc(-c2ccc(-c3ccc(N(c4ccc(-c5ccc6c(c5)oc5ccccc56)cc4)c4ccc(-c5cccc6c5oc5ccccc56)cc4)cc3)cc2)cc1 ; c1ccc2c(c1)oc1c(-c3ccc(N(c4ccc(-c5cccc6c5oc5ccccc56)cc4)c4ccc(-c5cccc6c5oc5ccccc56)cc4)cc3)cccc12]

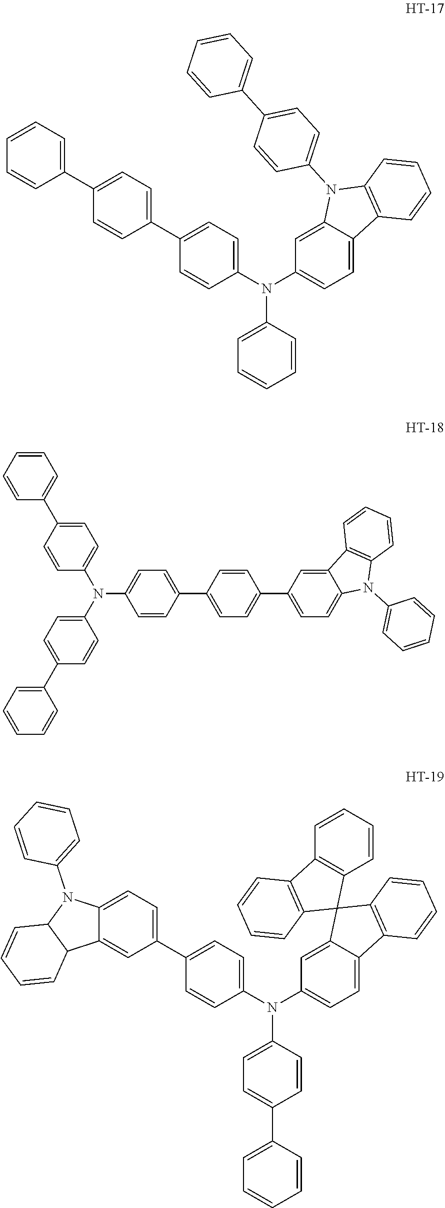 [SMILES: c1ccc(-c2ccc(-c3ccc(N(c4ccccc4)c4ccc5c6ccccc6n(-c6ccc(-c7ccccc7)cc6)c5c4)cc3)cc2)cc1 ; c1ccc(-c2ccc(N(c3ccc(-c4ccccc4)cc3)c3ccc(-c4ccc(-c5ccc6c(c5)c5ccccc5n6-c5ccccc5)cc4)cc3)cc2)cc1 ; C1=CC2c3cc(-c4ccc(N(c5ccc(-c6ccccc6)cc5)c5ccc6c(c5)C5(c7ccccc7-c7ccccc75)c5ccccc5-6)cc4)ccc3N(c3ccccc3)C2C=C1]

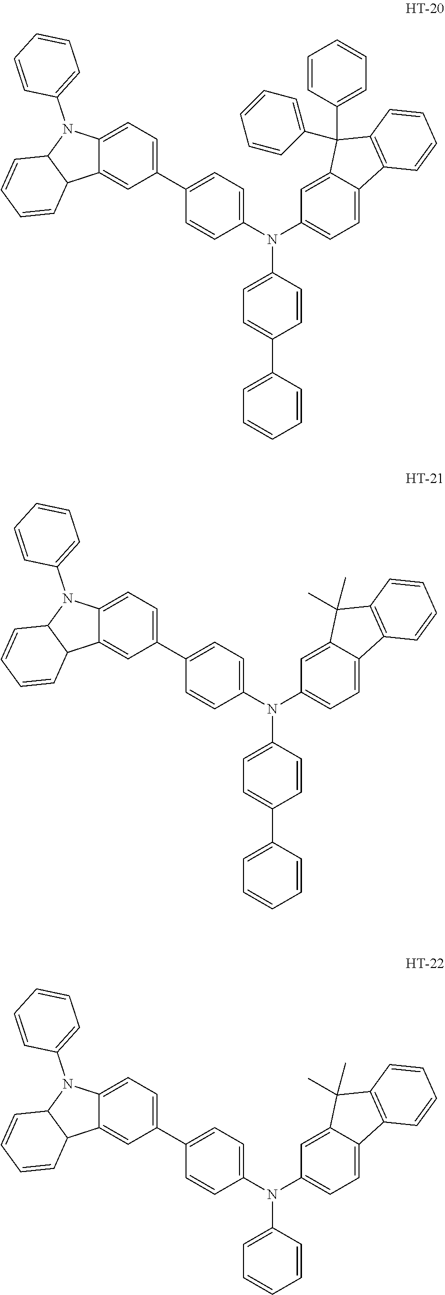 [SMILES: C1=CC2c3cc(-c4ccc(N(c5ccc(-c6ccccc6)cc5)c5ccc6c(c5)C(c5ccccc5)(c5ccccc5)c5ccccc5-6)cc4)ccc3N(c3ccccc3)C2C=C1 ; CC1(C)c2ccccc2-c2ccc(N(c3ccc(-c4ccccc4)cc3)c3ccc(-c4ccc5c(c4)C4C=CC=CC4N5c4ccccc4)cc3)cc21 ; CC1(C)c2ccccc2-c2ccc(N(c3ccccc3)c3ccc(-c4ccc5c(c4)C4C=CC=CC4N5c4ccccc4)cc3)cc21]

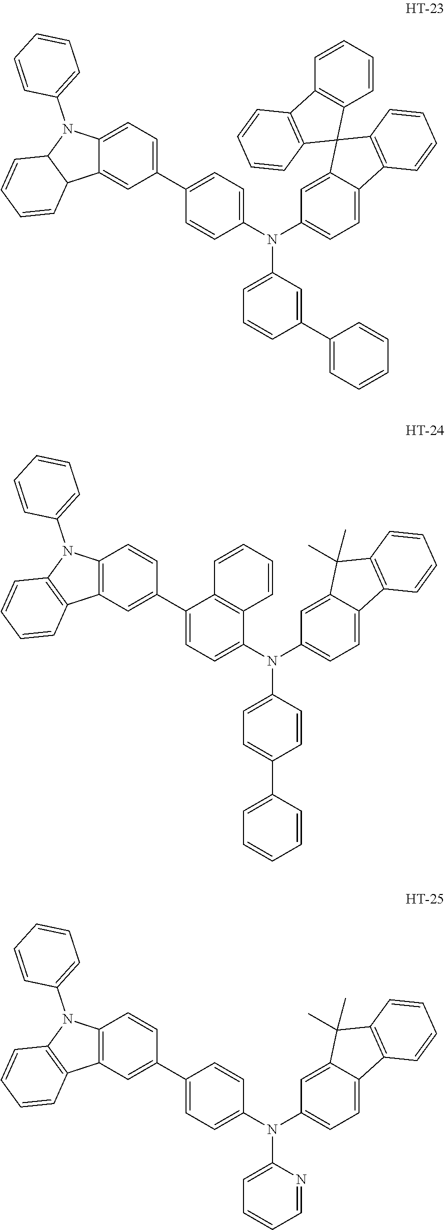 [SMILES: C1=CC2c3cc(-c4ccc(N(c5cccc(-c6ccccc6)c5)c5ccc6c(c5)C5(c7ccccc7-c7ccccc75)c5ccccc5-6)cc4)ccc3N(c3ccccc3)C2C=C1 ; CC1(C)c2ccccc2-c2ccc(N(c3ccc(-c4ccccc4)cc3)c3ccc(-c4ccc5c(c4)c4ccccc4n5-c4ccccc4)c4ccccc34)cc21 ; CC1(C)c2ccccc2-c2ccc(N(c3ccc(-c4ccc5c(c4)c4ccccc4n5-c4ccccc4)cc3)c3ccccn3)cc21]

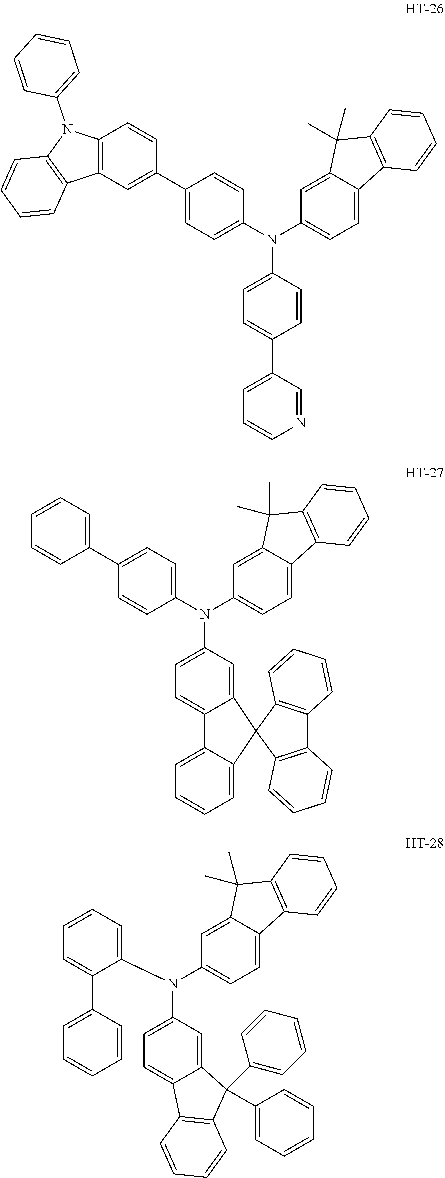

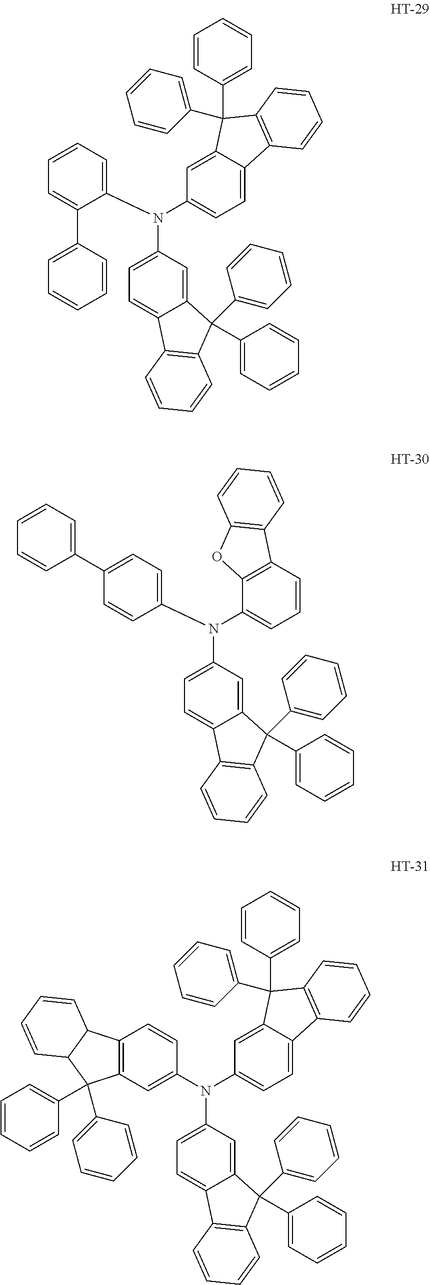 [SMILES: c1ccc(-c2ccccc2N(c2ccc3c(c2)C(c2ccccc2)(c2ccccc2)c2ccccc2-3)c2ccc3c(c2)C(c2ccccc2)(c2ccccc2)c2ccccc2-3)cc1 ; c1ccc(-c2ccc(N(c3ccc4c(c3)C(c3ccccc3)(c3ccccc3)c3ccccc3-4)c3cccc4c3oc3ccccc34)cc2)cc1 ; C1=CC2c3ccc(N(c4ccc5c(c4)C(c4ccccc4)(c4ccccc4)c4ccccc4-5)c4ccc5c(c4)C(c4ccccc4)(c4ccccc4)c4ccccc4-5)cc3C(c3ccccc3)(c3ccccc3)C2C=C1]

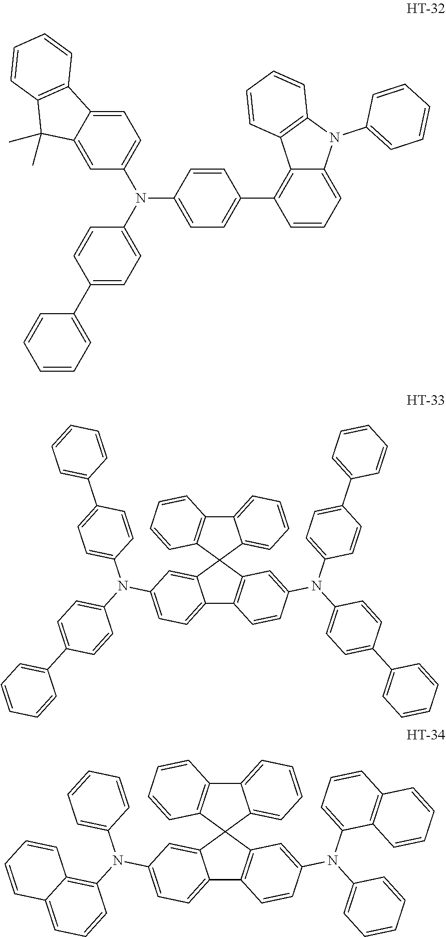 [SMILES: CC1(C)c2ccccc2-c2ccc(N(c3ccc(-c4ccccc4)cc3)c3ccc(-c4cccc5c4c4ccccc4n5-c4ccccc4)cc3)cc21 ; c1ccc(-c2ccc(N(c3ccc(-c4ccccc4)cc3)c3ccc4c(c3)C3(c5ccccc5-c5ccccc53)c3cc(N(c5ccc(-c6ccccc6)cc5)c5ccc(-c6ccccc6)cc5)ccc3-4)cc2)cc1 ; c1ccc(N(c2ccc3c(c2)C2(c4ccccc4-c4ccccc42)c2cc(N(c4ccccc4)c4cccc5ccccc45)ccc2-3)c2cccc3ccccc23)cc1]

The hole injection layer is located between the anode and the hole transport layer. The hole injection layer may be made of a single compound, or a combination of a plurality of compounds. For example, the hole injection layer may be one or more compounds as shown in the above HT-1 to HT-34, or one or more compounds as shown in the following HI-1 to HI-3, or one or more compounds as shown in the above HT-1 to HT-34 doped with one or more compounds as shown in the following HI-1 to HI-3.

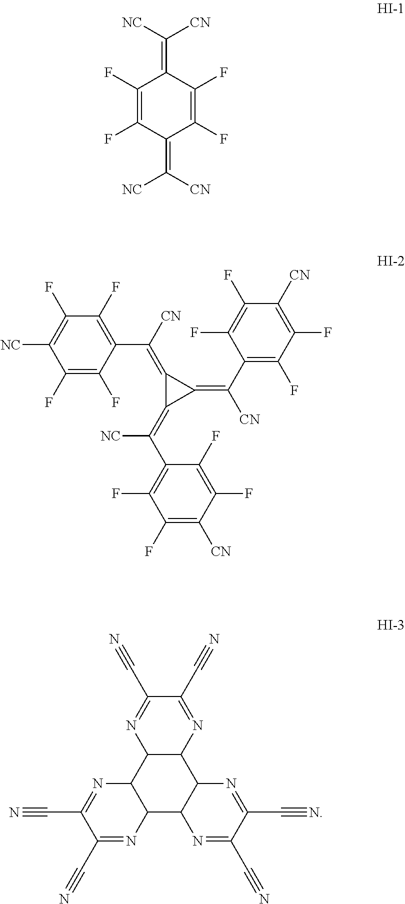 [SMILES: N#CC(C#N)=c1c(F)c(F)c(=C(C#N)C#N)c(F)c1F ; N#CC(=C1C(=C(C#N)c2c(F)c(F)c(C#N)c(F)c2F)C1=C(C#N)c1c(F)c(F)c(C#N)c(F)c1F)c1c(F)c(F)c(C#N)c(F)c1F ; N#CC1=NC2C3N=C(C#N)C(C#N)=NC3C3N=C(C#N)C(C#N)=NC3C2N=C1C#N]

The luminescent layer includes a luminescent dye (namely, a dopant) which may emit different wavelength spectrum, and may further include a host material (Host) at the same time. The luminescent layer may be a single-color luminescent layer emitting red, green, blue and other single-color light. Multiple different colors of single-color luminescent layers may be arranged planarly according to pixel graphics and may be also piled together to form a colorful luminescent layer. Different colors of luminescent layers may be separated mutually or collected when piled together. The luminescent layer may be a single colorful luminescent layer emitting red, green, blue and other different colors of light.

According to different technologies, the luminescent layer may be made of fluorescent electroluminescent materials, phosphorescent electroluminescent materials. TADF luminescent materials and the like. A single luminescent technology or a combination of multiple different luminescent technologies may be used in an OLED device. These different luminescent materials classified by technologies may emit the same color of light, and also may emit different colors of light.

In one aspect of the present invention, the fluorescent electroluminescent technology is used in the luminescent layer. The fluorescent host material of the luminescent layer may be selected from, but not limited to one or more combinations listed in BFH-1 to BFH-17.

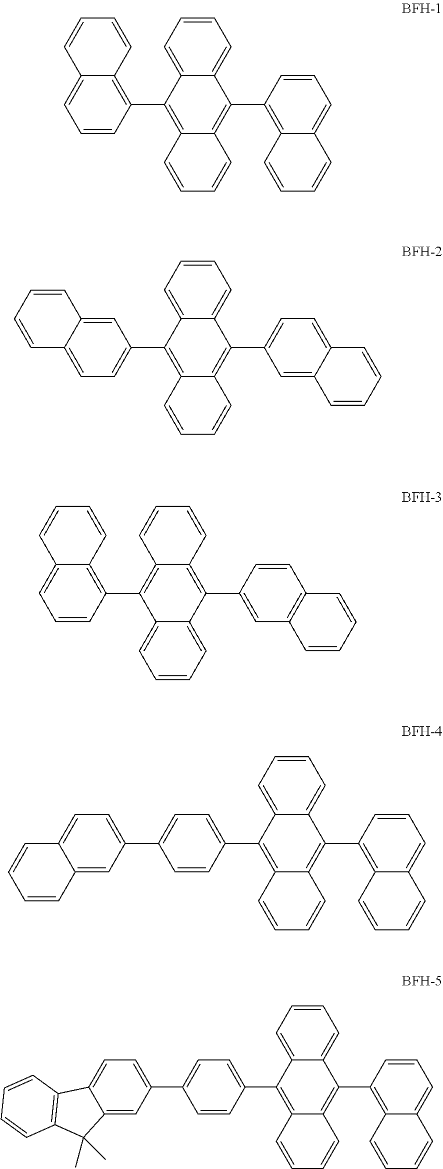

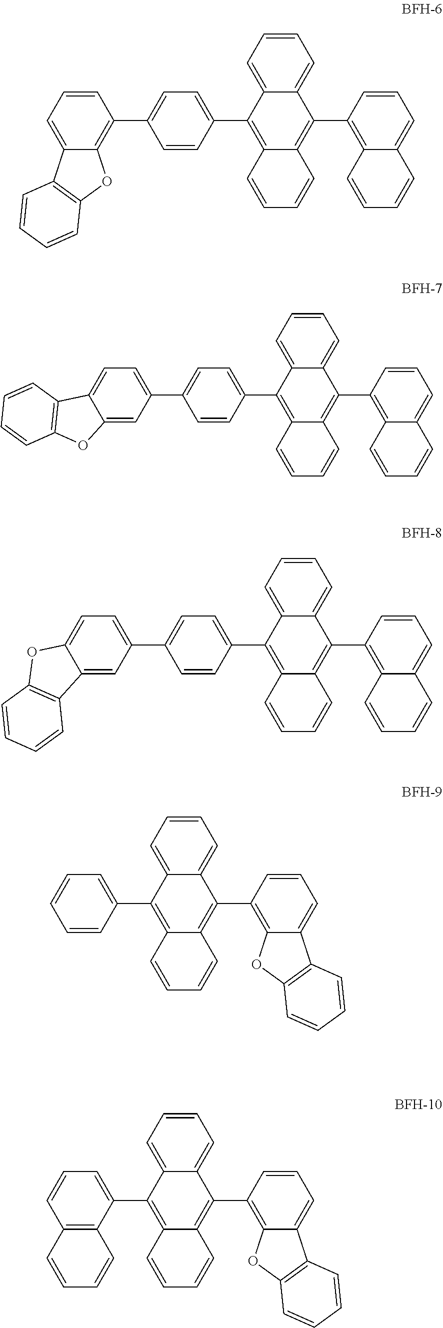 [SMILES: c1ccc2c(-c3c4ccccc4c(-c4ccc(-c5cccc6c5oc5ccccc56)cc4)c4ccccc34)cccc2c1 ; c1ccc2c(-c3c4ccccc4c(-c4ccc(-c5ccc6c(c5)oc5ccccc56)cc4)c4ccccc34)cccc2c1 ; c1ccc2c(-c3c4ccccc4c(-c4ccc(-c5ccc6oc7ccccc7c6c5)cc4)c4ccccc34)cccc2c1 ; c1ccc(-c2c3ccccc3c(-c3cccc4c3oc3ccccc34)c3ccccc23)cc1 ; c1ccc2c(-c3c4ccccc4c(-c4cccc5c4oc4ccccc45)c4ccccc34)cccc2c1]

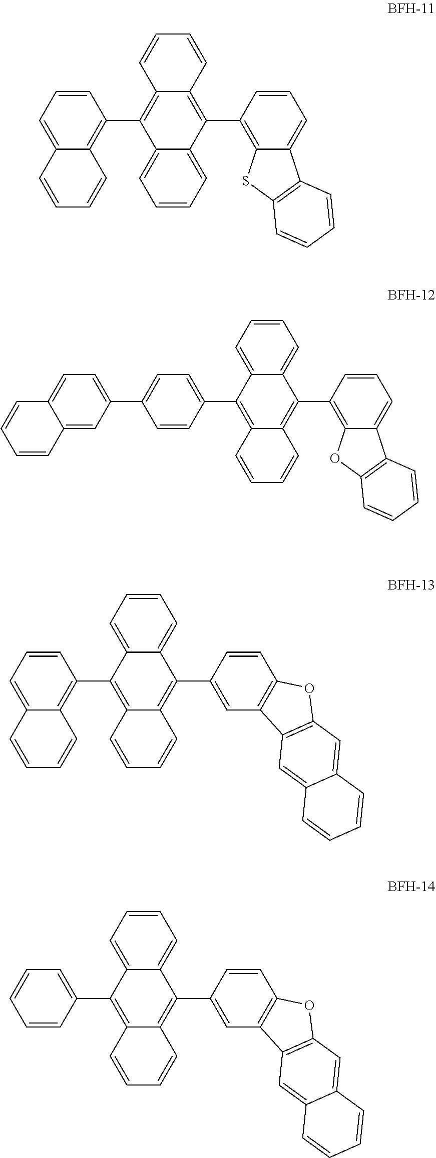 [SMILES: c1ccc2c(-c3c4ccccc4c(-c4cccc5c4sc4ccccc45)c4ccccc34)cccc2c1 ; c1ccc2cc(-c3ccc(-c4c5ccccc5c(-c5cccc6c5oc5ccccc56)c5ccccc45)cc3)ccc2c1 ; c1ccc2cc3c(cc2c1)oc1ccc(-c2c4ccccc4c(-c4cccc5ccccc45)c4ccccc24)cc13 ; c1ccc(-c2c3ccccc3c(-c3ccc4oc5cc6ccccc6cc5c4c3)c3ccccc23)cc1]

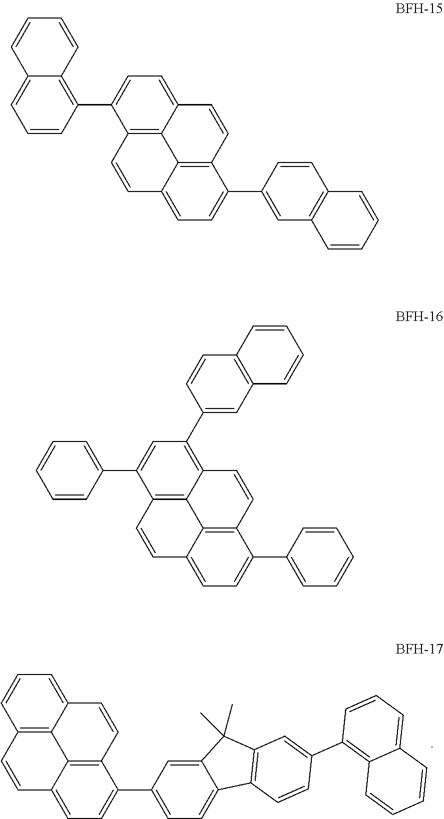

In one aspect of the present invention, the fluorescent electroluminescent technology is used in the luminescent layer. The fluorescent dopant of the luminescent layer may be selected from, but not limited to one or more combinations listed in BFD-1 to BFD-12.

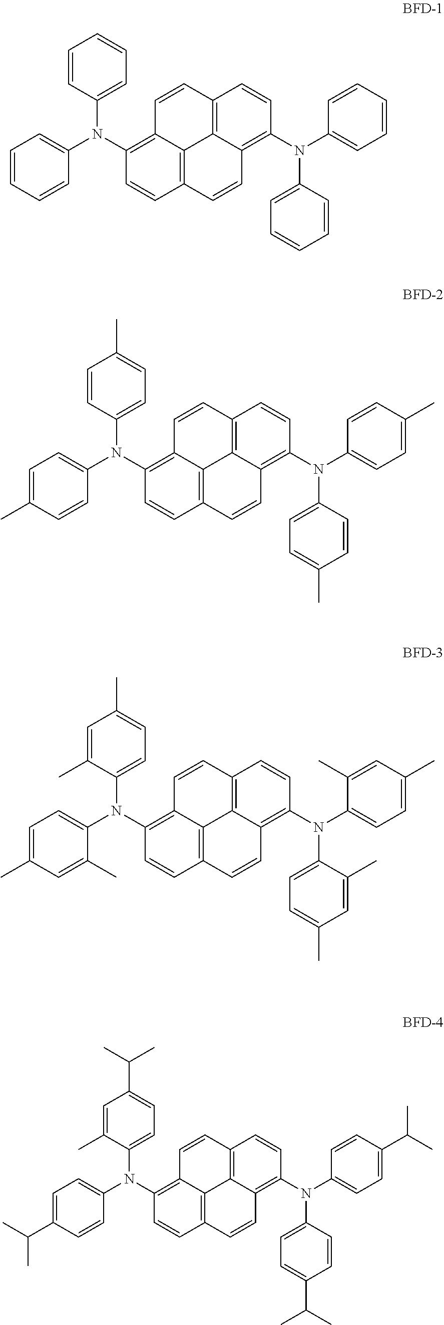

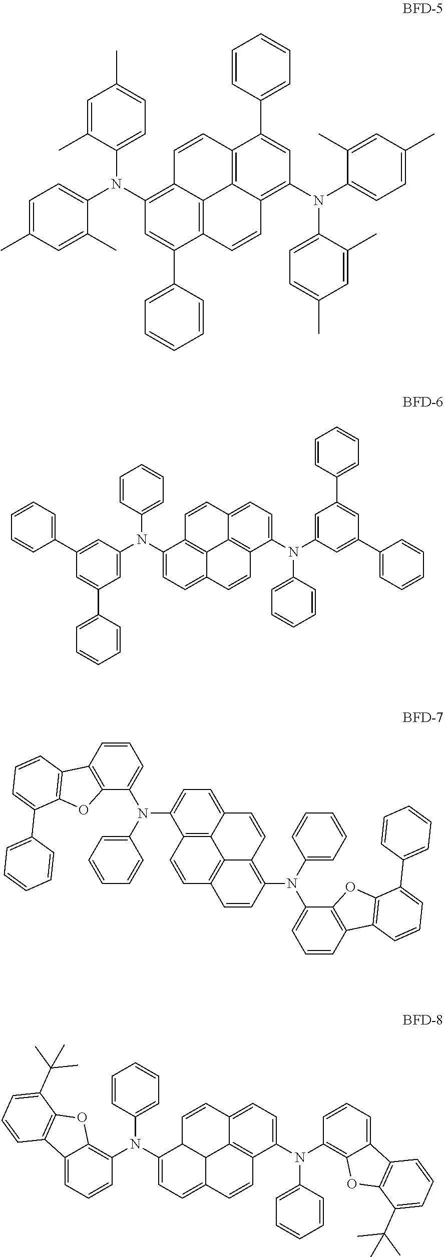

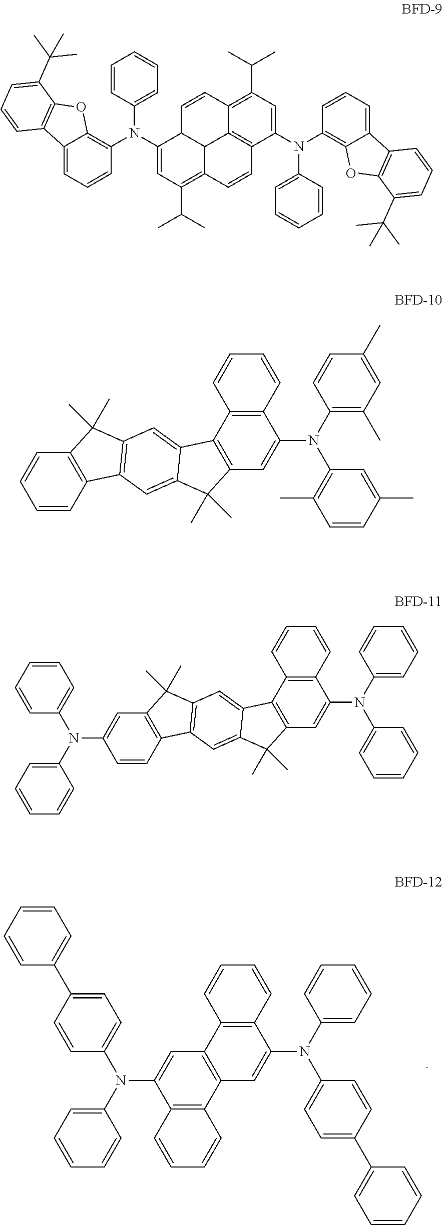 [SMILES: CC(C)C1=C2C=Cc3c(N(c4ccccc4)c4cccc5c4oc4c(C(C)(C)C)cccc45)cc(C(C)C)c4c3C2C(C=C4)C(N(c2ccccc2)c2cccc3c2oc2c(C(C)(C)C)cccc23)=C1 ; Cc1ccc(N(c2cc(C)ccc2C)c2cc3c(c4ccccc24)-c2cc4c(cc2C3(C)C)-c2ccccc2C4(C)C)c(C)c1 ; CC1(C)c2cc(N(c3ccccc3)c3ccccc3)ccc2-c2cc3c(cc21)-c1c(cc(N(c2ccccc2)c2ccccc2)c2ccccc12)C3(C)C ; c1ccc(-c2ccc(N(c3ccccc3)c3cc4c5ccccc5c(N(c5ccccc5)c5ccc(-c6ccccc6)cc5)cc4c4ccccc34)cc2)cc1]

In one aspect of the present invention, the phosphorescent electroluminescent technology is used in the luminescent layer. The fluorescent host material of the luminescent layer may be selected from, but not limited to one or more compounds listed in GPH-1 to GPH-80.

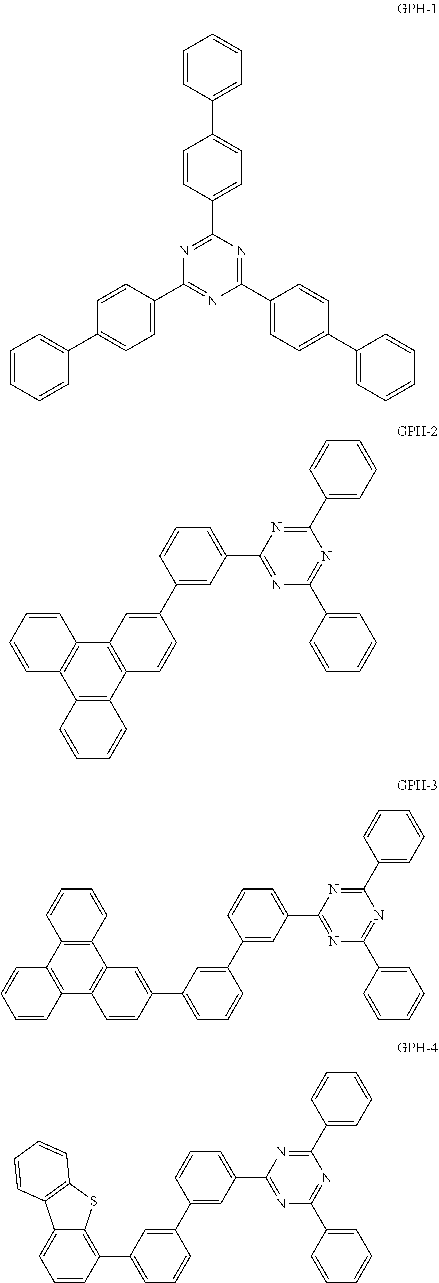 [SMILES: c1ccc(-c2ccc(-c3nc(-c4ccc(-c5ccccc5)cc4)nc(-c4ccc(-c5ccccc5)cc4)n3)cc2)cc1 ; c1ccc(-c2nc(-c3ccccc3)nc(-c3cccc(-c4ccc5c6ccccc6c6ccccc6c5c4)c3)n2)cc1 ; c1ccc(-c2nc(-c3ccccc3)nc(-c3cccc(-c4cccc(-c5ccc6c7ccccc7c7ccccc7c6c5)c4)c3)n2)cc1 ; c1ccc(-c2nc(-c3ccccc3)nc(-c3cccc(-c4cccc(-c5cccc6c5sc5ccccc56)c4)c3)n2)cc1]

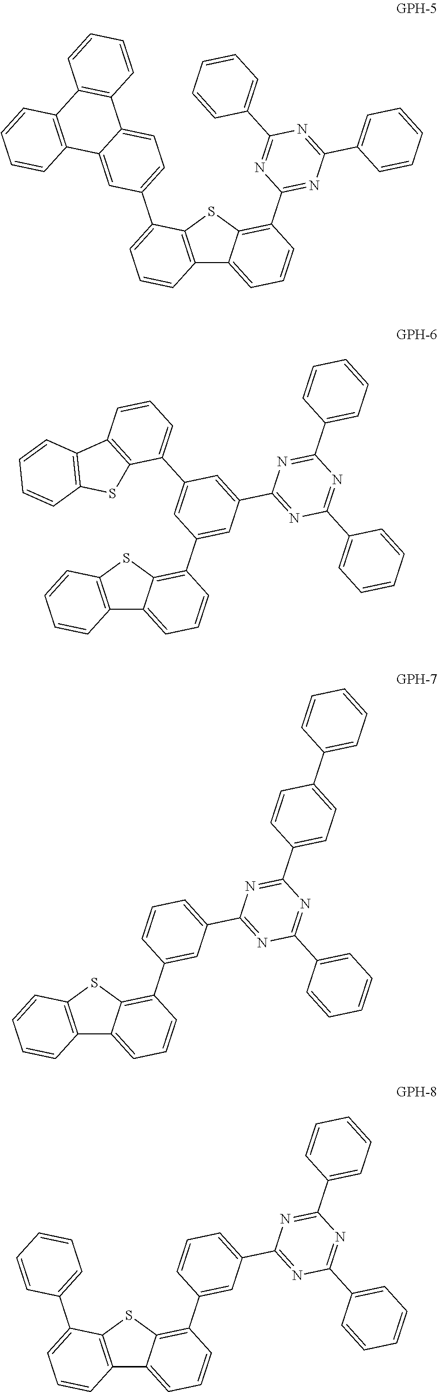 [SMILES: c1ccc(-c2nc(-c3ccccc3)nc(-c3cccc4c3sc3c(-c5ccc6c7ccccc7c7ccccc7c6c5)cccc34)n2)cc1 ; c1ccc(-c2nc(-c3ccccc3)nc(-c3cc(-c4cccc5c4sc4ccccc45)cc(-c4cccc5c4sc4ccccc45)c3)n2)cc1 ; c1ccc(-c2ccc(-c3nc(-c4ccccc4)nc(-c4cccc(-c5cccc6c5sc5ccccc56)c4)n3)cc2)cc1 ; c1ccc(-c2nc(-c3ccccc3)nc(-c3cccc(-c4cccc5c4sc4c(-c6ccccc6)cccc45)c3)n2)cc1]

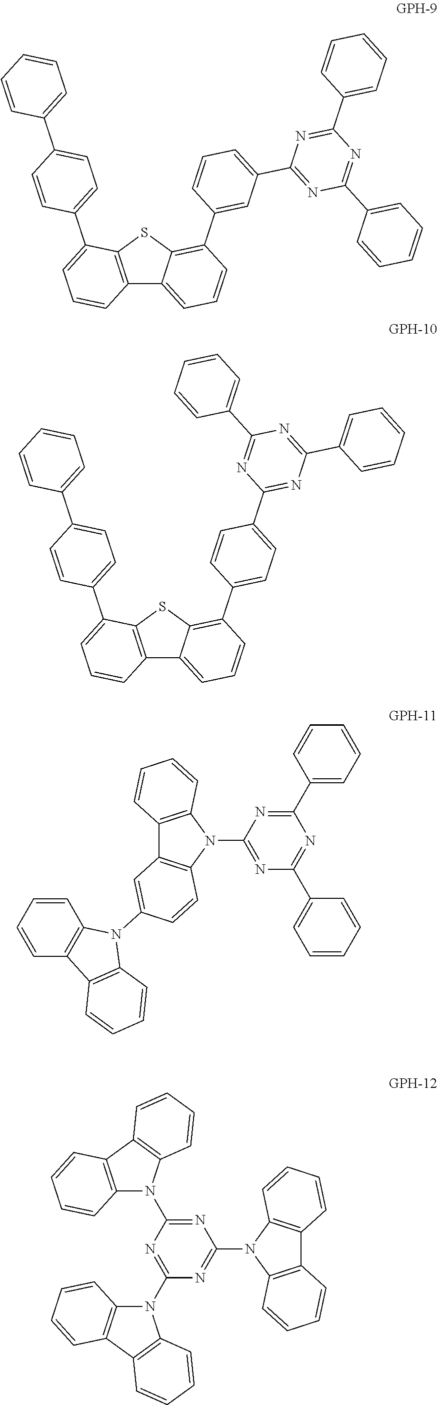 [SMILES: c1ccc(-c2ccc(-c3cccc4c3sc3c(-c5cccc(-c6nc(-c7ccccc7)nc(-c7ccccc7)n6)c5)cccc34)cc2)cc1 ; c1ccc(-c2ccc(-c3cccc4c3sc3c(-c5ccc(-c6nc(-c7ccccc7)nc(-c7ccccc7)n6)cc5)cccc34)cc2)cc1 ; c1ccc(-c2nc(-c3ccccc3)nc(-n3c4ccccc4c4cc(-n5c6ccccc6c6ccccc65)ccc43)n2)cc1 ; c1ccc2c(c1)c1ccccc1n2-c1nc(-n2c3ccccc3c3ccccc32)nc(-n2c3ccccc3c3ccccc32)n1]

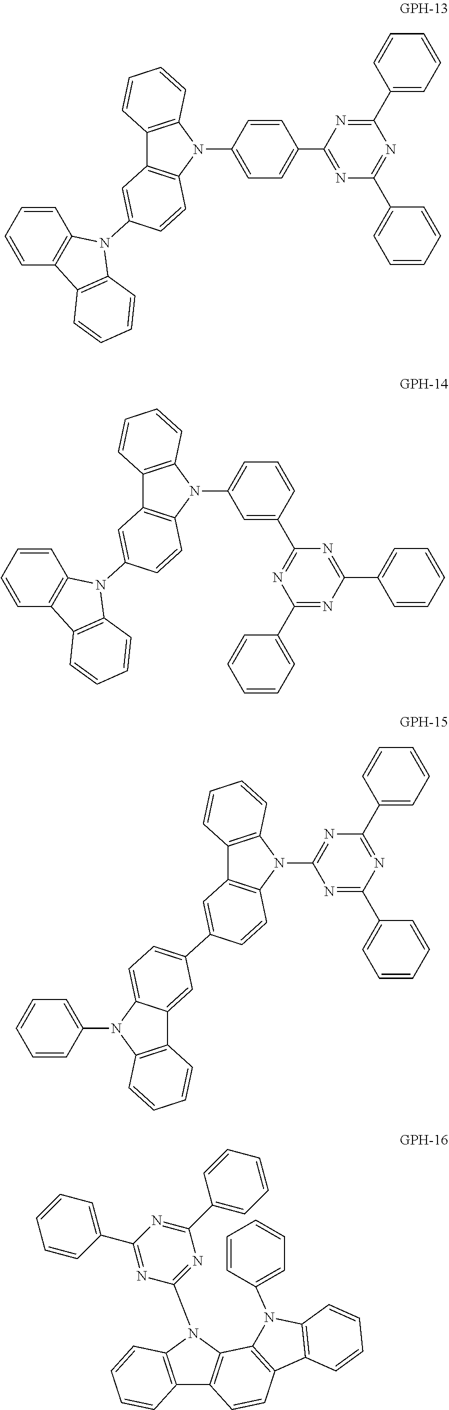 [SMILES: c1ccc(-c2nc(-c3ccccc3)nc(-c3ccc(-n4c5ccccc5c5cc(-n6c7ccccc7c7ccccc76)ccc54)cc3)n2)cc1 ; c1ccc(-c2nc(-c3ccccc3)nc(-c3cccc(-n4c5ccccc5c5cc(-n6c7ccccc7c7ccccc76)ccc54)c3)n2)cc1 ; c1ccc(-c2nc(-c3ccccc3)nc(-n3c4ccccc4c4cc(-c5ccc6c(c5)c5ccccc5n6-c5ccccc5)ccc43)n2)cc1 ; c1ccc(-c2nc(-c3ccccc3)nc(-n3c4ccccc4c4ccc5c6ccccc6n(-c6ccccc6)c5c43)n2)cc1]

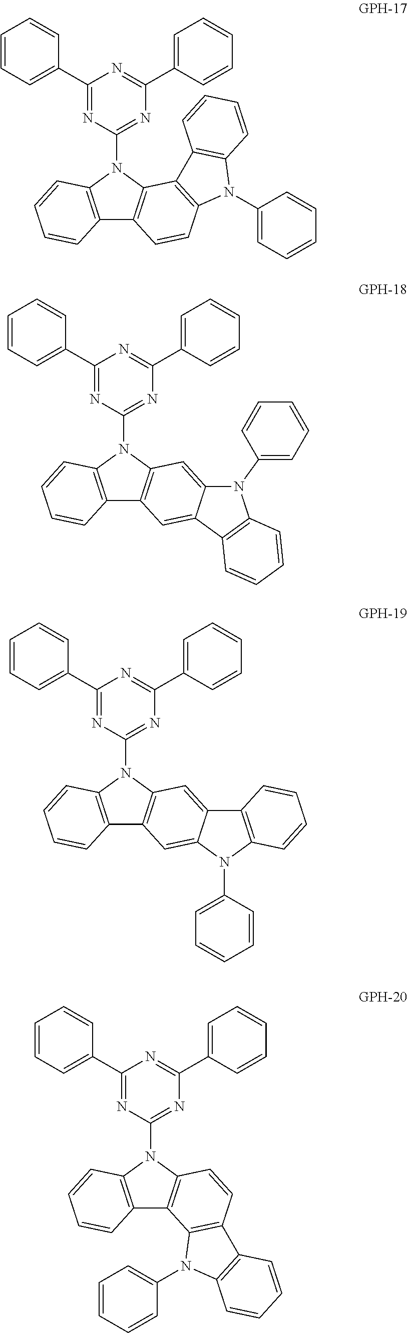

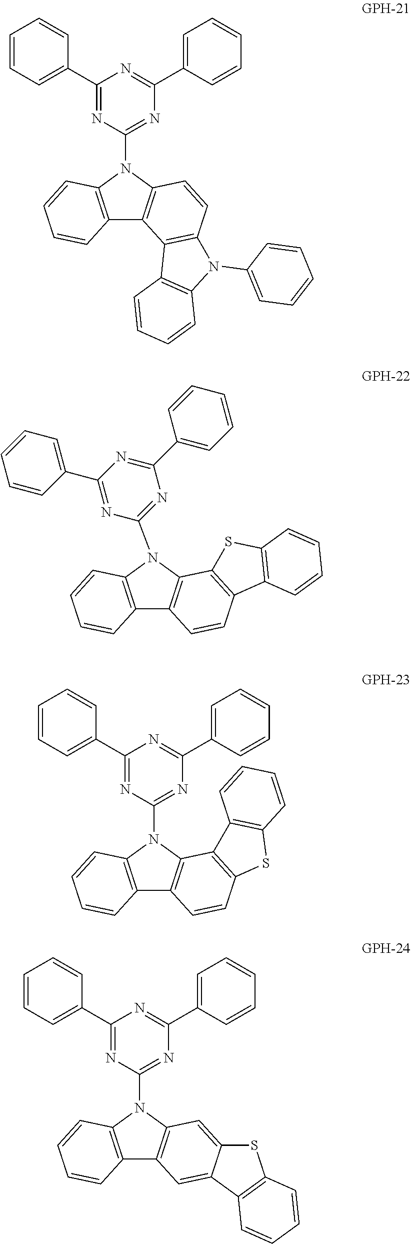 [SMILES: c1ccc(-c2nc(-c3ccccc3)nc(-n3c4ccccc4c4c5c6ccccc6n(-c6ccccc6)c5ccc43)n2)cc1 ; c1ccc(-c2nc(-c3ccccc3)nc(-n3c4ccccc4c4ccc5c6ccccc6sc5c43)n2)cc1 ; c1ccc(-c2nc(-c3ccccc3)nc(-n3c4ccccc4c4ccc5sc6ccccc6c5c43)n2)cc1 ; c1ccc(-c2nc(-c3ccccc3)nc(-n3c4ccccc4c4cc5c(cc43)sc3ccccc35)n2)cc1]

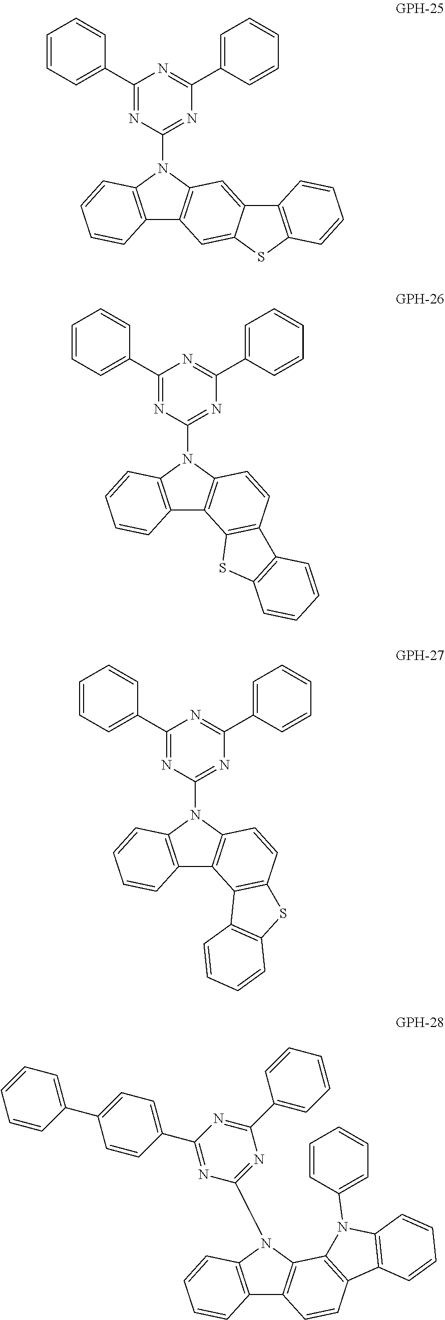 [SMILES: c1ccc(-c2nc(-c3ccccc3)nc(-n3c4ccccc4c4cc5sc6ccccc6c5cc43)n2)cc1 ; c1ccc(-c2nc(-c3ccccc3)nc(-n3c4ccccc4c4c5sc6ccccc6c5ccc43)n2)cc1 ; c1ccc(-c2nc(-c3ccccc3)nc(-n3c4ccccc4c4c5c(ccc43)sc3ccccc35)n2)cc1 ; c1ccc(-c2ccc(-c3nc(-c4ccccc4)nc(-n4c5ccccc5c5ccc6c7ccccc7n(-c7ccccc7)c6c54)n3)cc2)cc1]

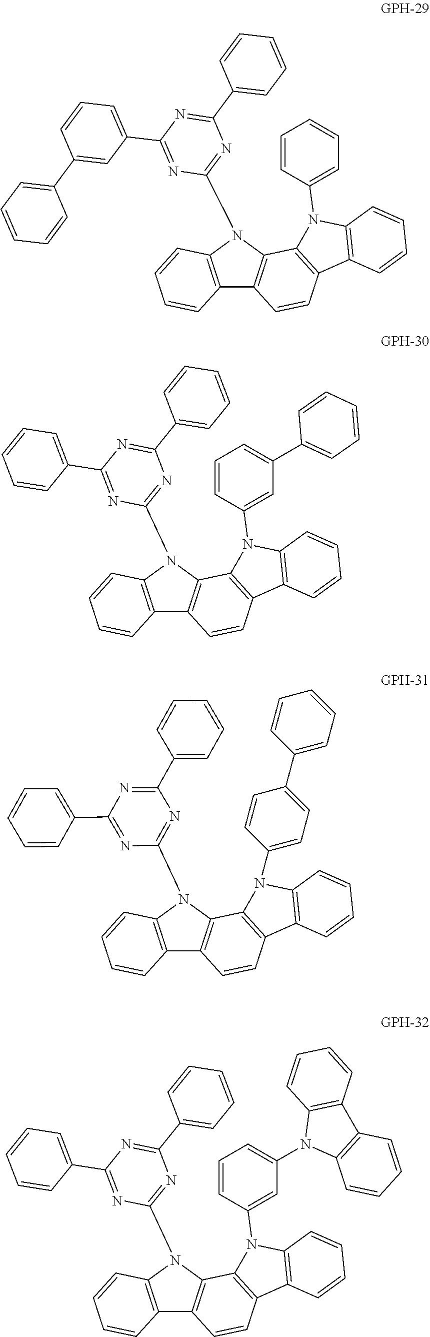 [SMILES: c1ccc(-c2cccc(-c3nc(-c4ccccc4)nc(-n4c5ccccc5c5ccc6c7ccccc7n(-c7ccccc7)c6c54)n3)c2)cc1 ; c1ccc(-c2cccc(-n3c4ccccc4c4ccc5c6ccccc6n(-c6nc(-c7ccccc7)nc(-c7ccccc7)n6)c5c43)c2)cc1 ; c1ccc(-c2ccc(-n3c4ccccc4c4ccc5c6ccccc6n(-c6nc(-c7ccccc7)nc(-c7ccccc7)n6)c5c43)cc2)cc1 ; c1ccc(-c2nc(-c3ccccc3)nc(-n3c4ccccc4c4ccc5c6ccccc6n(-c6cccc(-n7c8ccccc8c8ccccc87)c6)c5c43)n2)cc1]

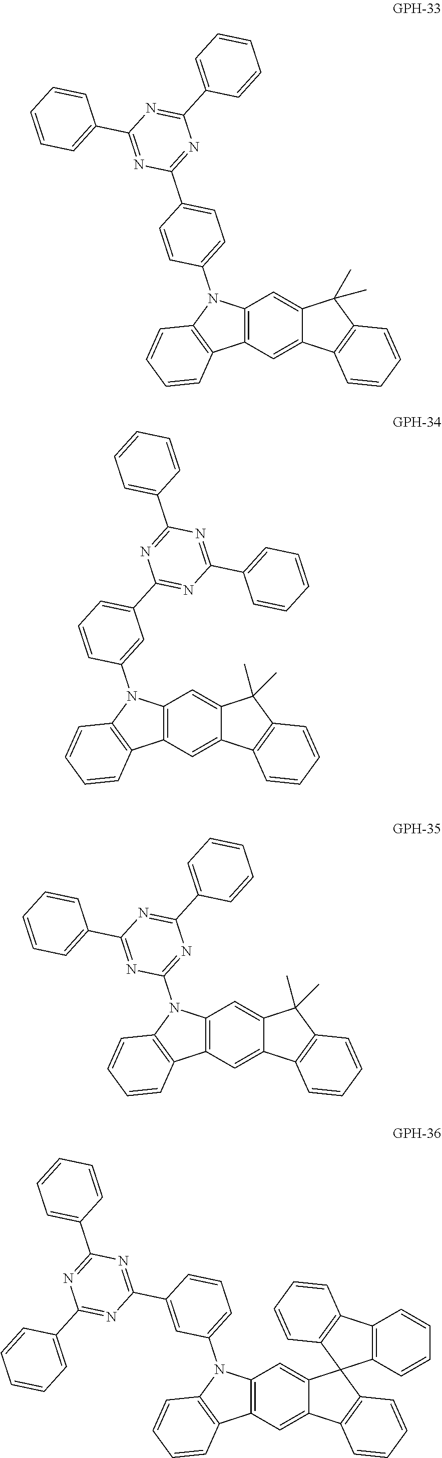 [SMILES: CC1(C)c2ccccc2-c2cc3c4ccccc4n(-c4ccc(-c5nc(-c6ccccc6)nc(-c6ccccc6)n5)cc4)c3cc21 ; CC1(C)c2ccccc2-c2cc3c4ccccc4n(-c4cccc(-c5nc(-c6ccccc6)nc(-c6ccccc6)n5)c4)c3cc21 ; CC1(C)c2ccccc2-c2cc3c4ccccc4n(-c4nc(-c5ccccc5)nc(-c5ccccc5)n4)c3cc21 ; c1ccc(-c2nc(-c3ccccc3)nc(-c3cccc(-n4c5ccccc5c5cc6c(cc54)C4(c5ccccc5-c5ccccc54)c4ccccc4-6)c3)n2)cc1]

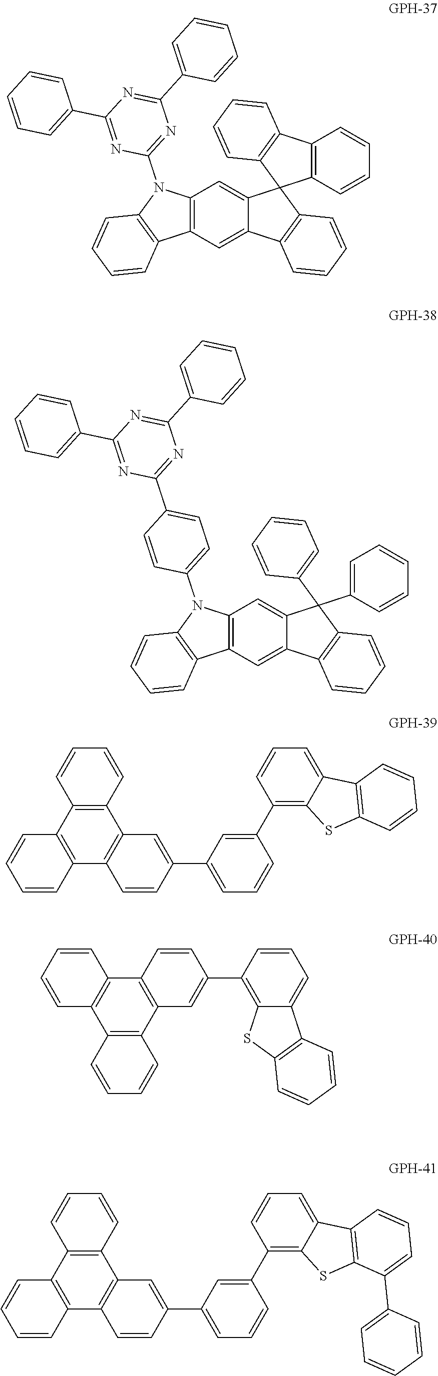 [SMILES: c1ccc(-c2nc(-c3ccccc3)nc(-n3c4ccccc4c4cc5c(cc43)C3(c4ccccc4-c4ccccc43)c3ccccc3-5)n2)cc1 ; c1ccc(-c2nc(-c3ccccc3)nc(-c3ccc(-n4c5ccccc5c5cc6c(cc54)C(c4ccccc4)(c4ccccc4)c4ccccc4-6)cc3)n2)cc1 ; c1cc(-c2ccc3c4ccccc4c4ccccc4c3c2)cc(-c2cccc3c2sc2ccccc23)c1 ; c1ccc2c(c1)sc1c(-c3ccc4c5ccccc5c5ccccc5c4c3)cccc12 ; c1ccc(-c2cccc3c2sc2c(-c4cccc(-c5ccc6c7ccccc7c7ccccc7c6c5)c4)cccc23)cc1]

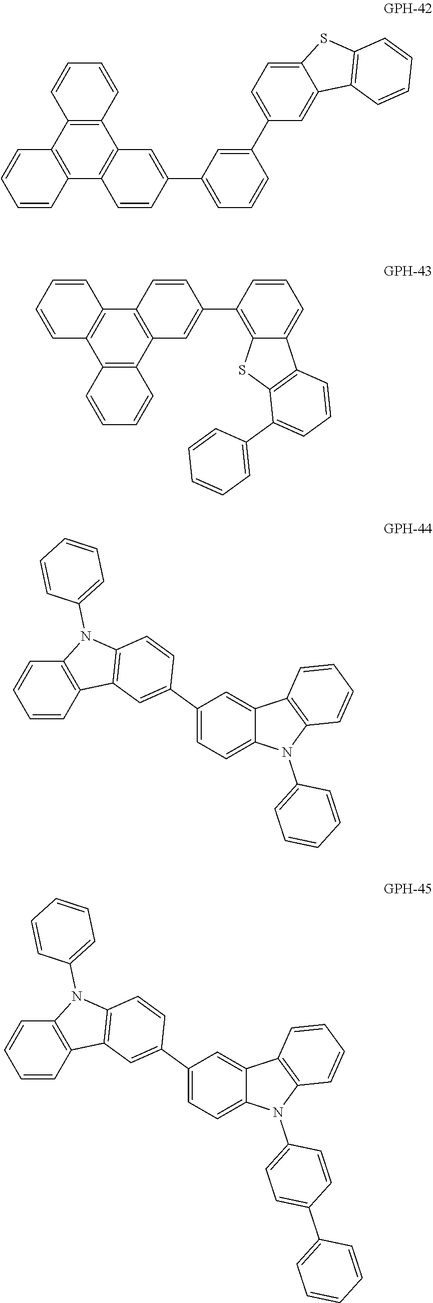 [SMILES: c1cc(-c2ccc3sc4ccccc4c3c2)cc(-c2ccc3c4ccccc4c4ccccc4c3c2)c1 ; c1ccc(-c2cccc3c2sc2c(-c4ccc5c6ccccc6c6ccccc6c5c4)cccc23)cc1 ; c1ccc(-n2c3ccccc3c3cc(-c4ccc5c(c4)c4ccccc4n5-c4ccccc4)ccc32)cc1 ; c1ccc(-c2ccc(-n3c4ccccc4c4cc(-c5ccc6c(c5)c5ccccc5n6-c5ccccc5)ccc43)cc2)cc1]

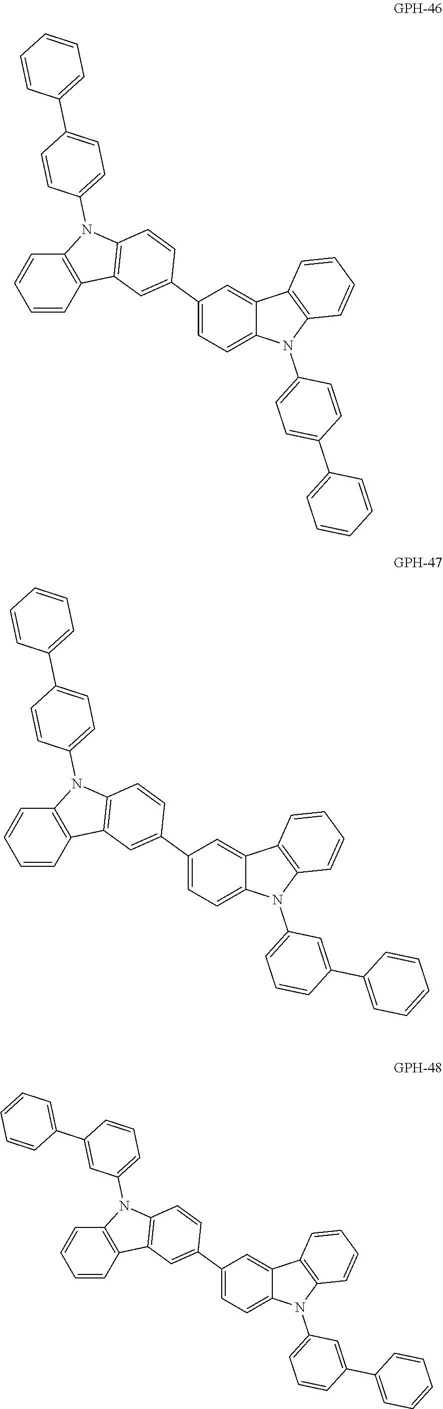 [SMILES: c1ccc(-c2ccc(-n3c4ccccc4c4cc(-c5ccc6c(c5)c5ccccc5n6-c5ccc(-c6ccccc6)cc5)ccc43)cc2)cc1 ; c1ccc(-c2ccc(-n3c4ccccc4c4cc(-c5ccc6c(c5)c5ccccc5n6-c5cccc(-c6ccccc6)c5)ccc43)cc2)cc1 ; c1ccc(-c2cccc(-n3c4ccccc4c4cc(-c5ccc6c(c5)c5ccccc5n6-c5cccc(-c6ccccc6)c5)ccc43)c2)cc1]

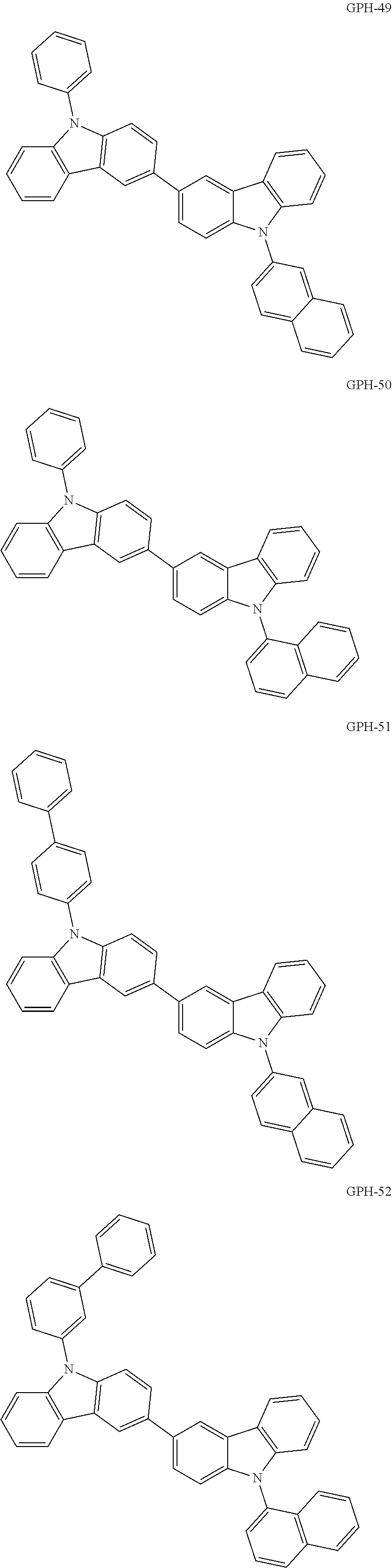 [SMILES: c1ccc(-n2c3ccccc3c3cc(-c4ccc5c(c4)c4ccccc4n5-c4ccc5ccccc5c4)ccc32)cc1 ; c1ccc(-n2c3ccccc3c3cc(-c4ccc5c(c4)c4ccccc4n5-c4cccc5ccccc45)ccc32)cc1 ; c1ccc(-c2ccc(-n3c4ccccc4c4cc(-c5ccc6c(c5)c5ccccc5n6-c5ccc6ccccc6c5)ccc43)cc2)cc1 ; c1ccc(-c2cccc(-n3c4ccccc4c4cc(-c5ccc6c(c5)c5ccccc5n6-c5cccc6ccccc56)ccc43)c2)cc1]

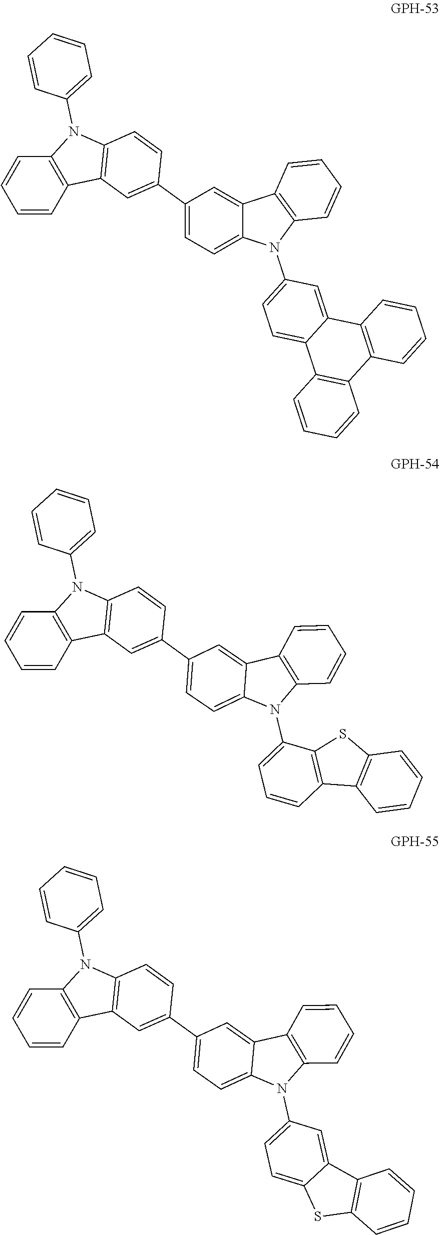 [SMILES: c1ccc(-n2c3ccccc3c3cc(-c4ccc5c(c4)c4ccccc4n5-c4ccc5c6ccccc6c6ccccc6c5c4)ccc32)cc1 ; c1ccc(-n2c3ccccc3c3cc(-c4ccc5c(c4)c4ccccc4n5-c4cccc5c4sc4ccccc45)ccc32)cc1 ; c1ccc(-n2c3ccccc3c3cc(-c4ccc5c(c4)c4ccccc4n5-c4ccc5sc6ccccc6c5c4)ccc32)cc1]

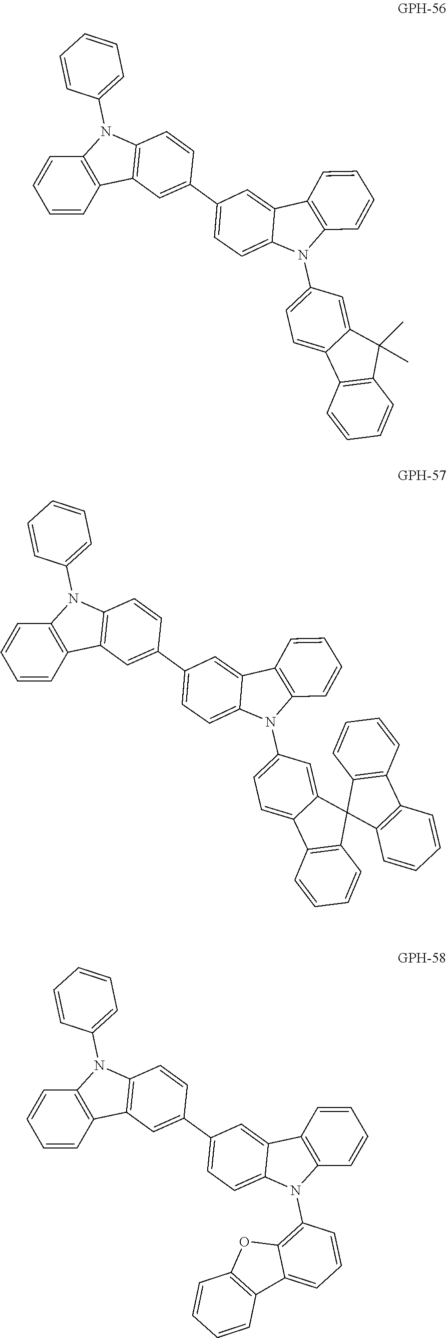 [SMILES: CC1(C)c2ccccc2-c2ccc(-n3c4ccccc4c4cc(-c5ccc6c(c5)c5ccccc5n6-c5ccccc5)ccc43)cc21 ; c1ccc(-n2c3ccccc3c3cc(-c4ccc5c(c4)c4ccccc4n5-c4ccc5c(c4)C4(c6ccccc6-c6ccccc64)c4ccccc4-5)ccc32)cc1 ; c1ccc(-n2c3ccccc3c3cc(-c4ccc5c(c4)c4ccccc4n5-c4cccc5c4oc4ccccc45)ccc32)cc1]

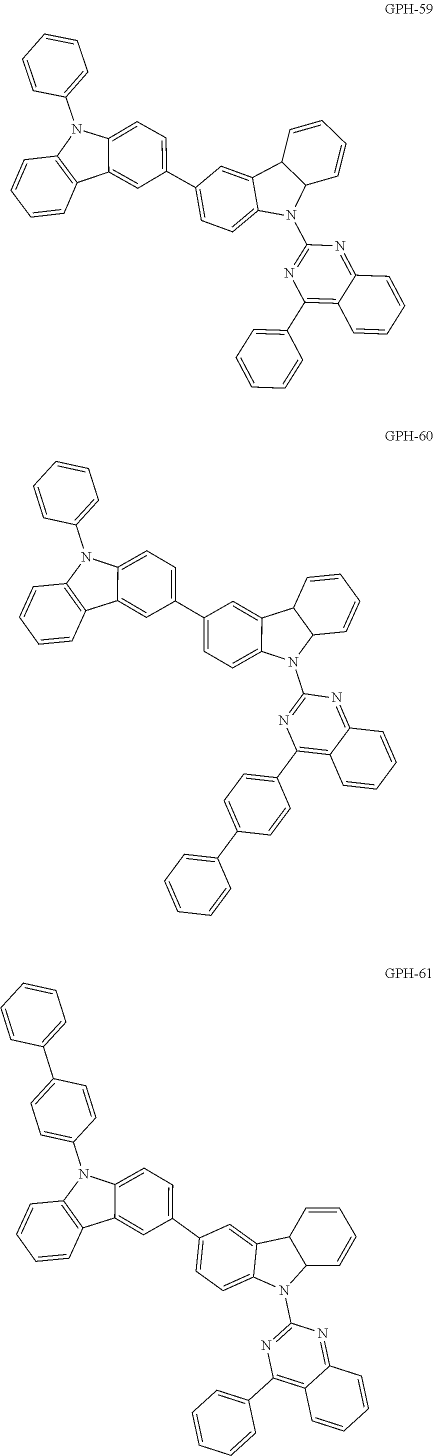 [SMILES: C1=CC2c3cc(-c4ccc5c(c4)c4ccccc4n5-c4ccccc4)ccc3N(c3nc(-c4ccccc4)c4ccccc4n3)C2C=C1 ; C1=CC2c3cc(-c4ccc5c(c4)c4ccccc4n5-c4ccccc4)ccc3N(c3nc(-c4ccc(-c5ccccc5)cc4)c4ccccc4n3)C2C=C1 ; C1=CC2c3cc(-c4ccc5c(c4)c4ccccc4n5-c4ccc(-c5ccccc5)cc4)ccc3N(c3nc(-c4ccccc4)c4ccccc4n3)C2C=C1]

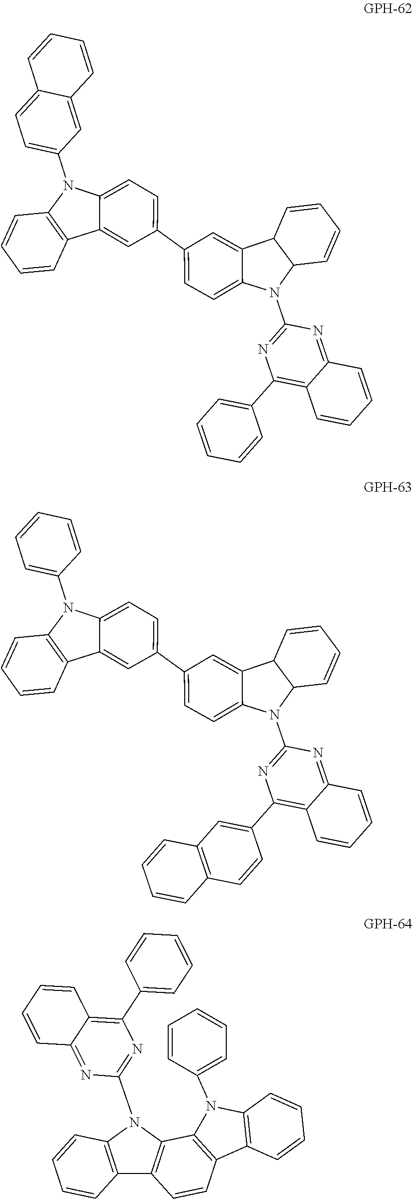

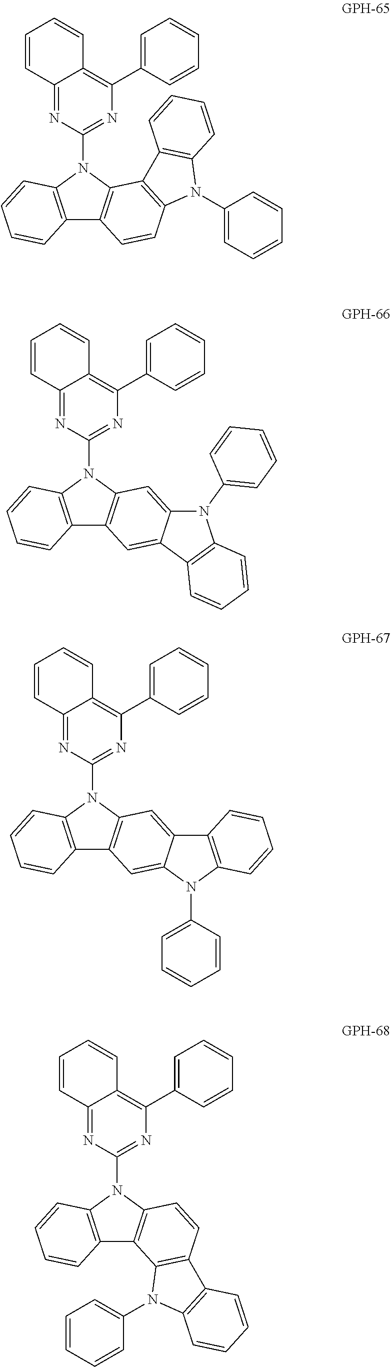 [SMILES: c1ccc(-c2nc(-n3c4ccccc4c4ccc5c(c6ccccc6n5-c5ccccc5)c43)nc3ccccc23)cc1 ; c1ccc(-c2nc(-n3c4ccccc4c4cc5c6ccccc6n(-c6ccccc6)c5cc43)nc3ccccc23)cc1 ; c1ccc(-c2nc(-n3c4ccccc4c4cc5c(cc43)c3ccccc3n5-c3ccccc3)nc3ccccc23)cc1 ; c1ccc(-c2nc(-n3c4ccccc4c4c3ccc3c5ccccc5n(-c5ccccc5)c34)nc3ccccc23)cc1]

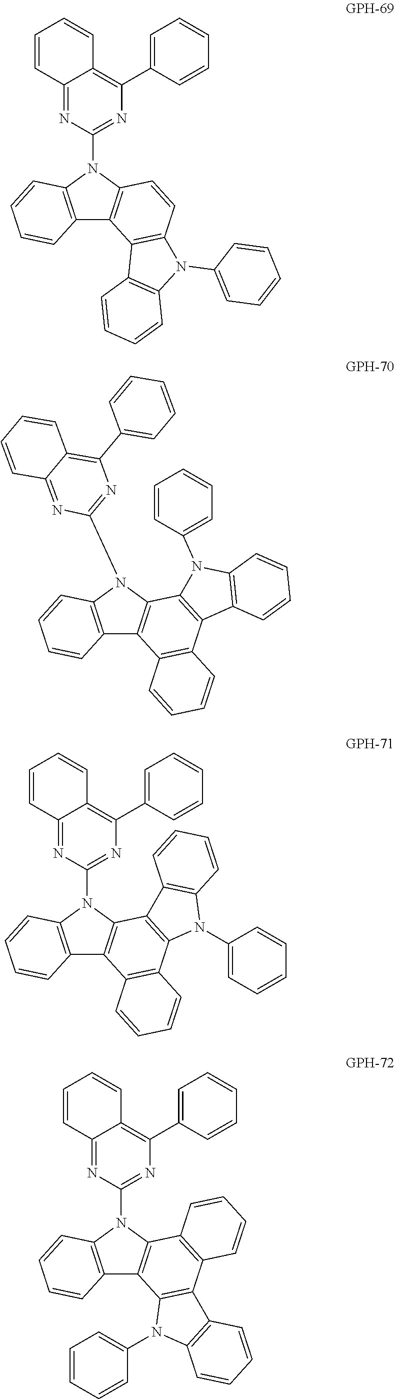

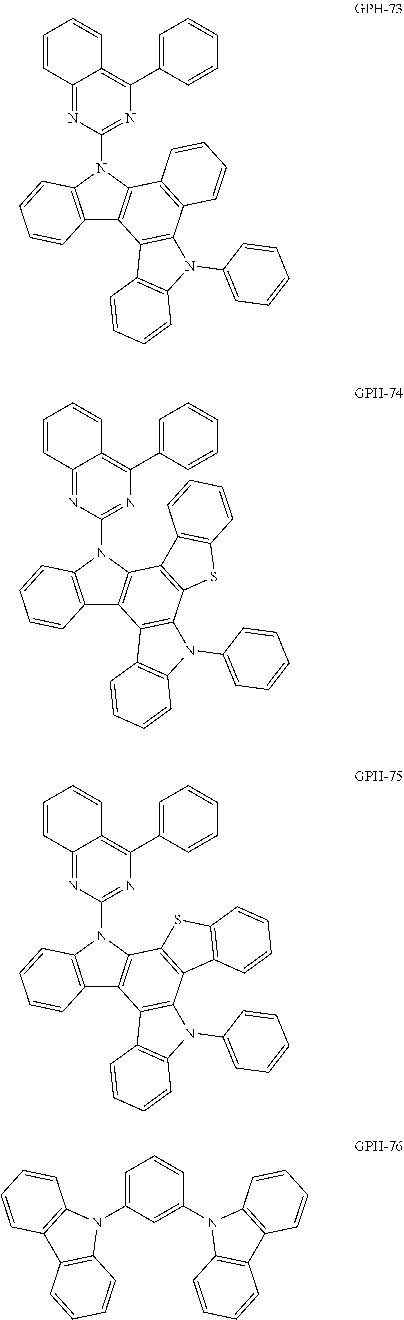

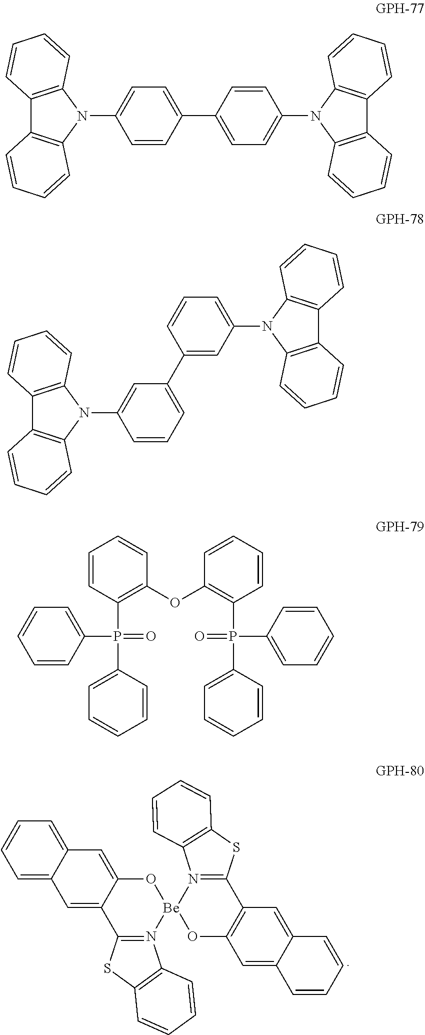

In one aspect of the present invention, the phosphorescent electroluminescent technology is used in the luminescent layer. The fluorescent dopant of the luminescent layer may be selected from, but not limited to one or more combinations listed in GPD-1 to GPD-47.

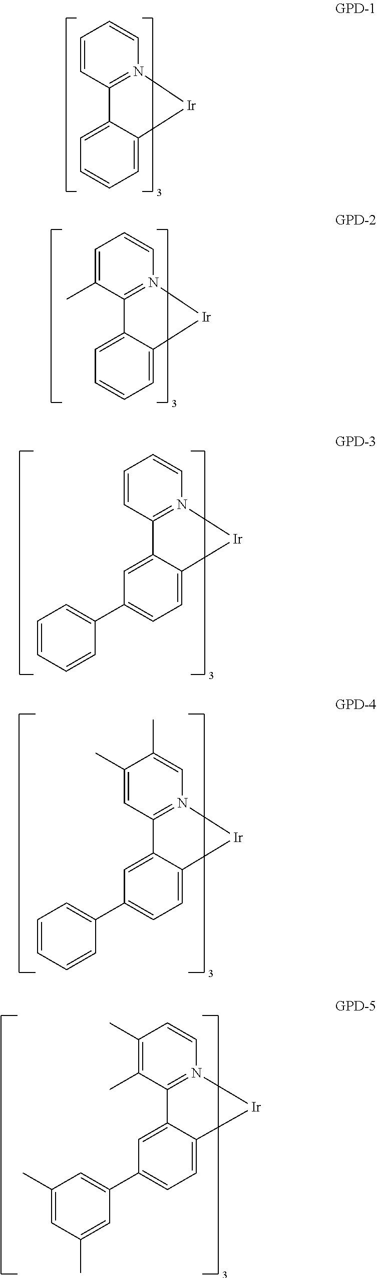

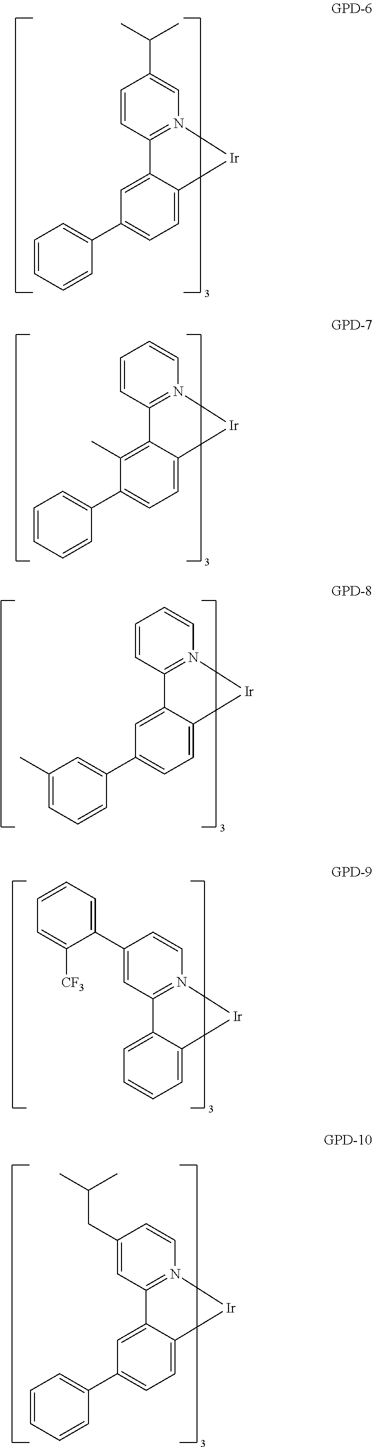

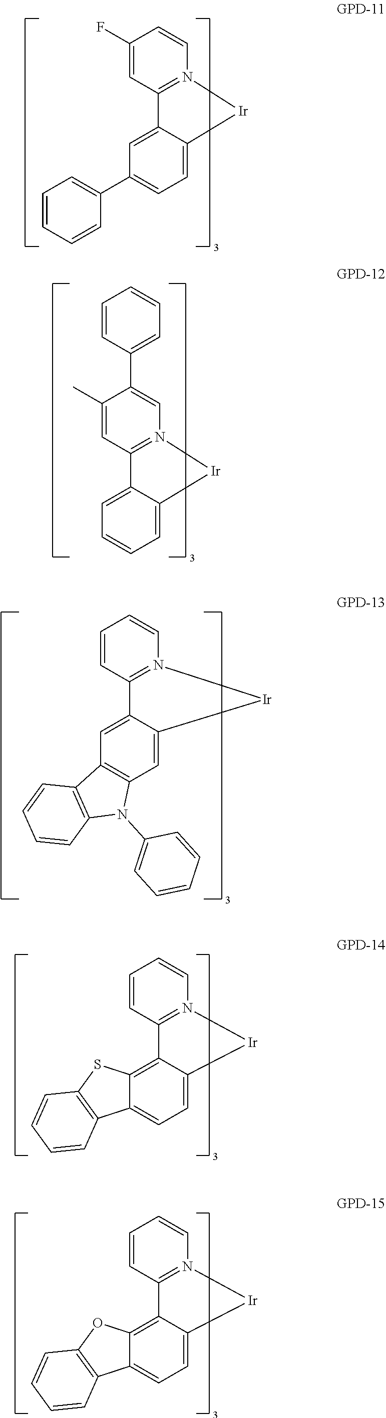

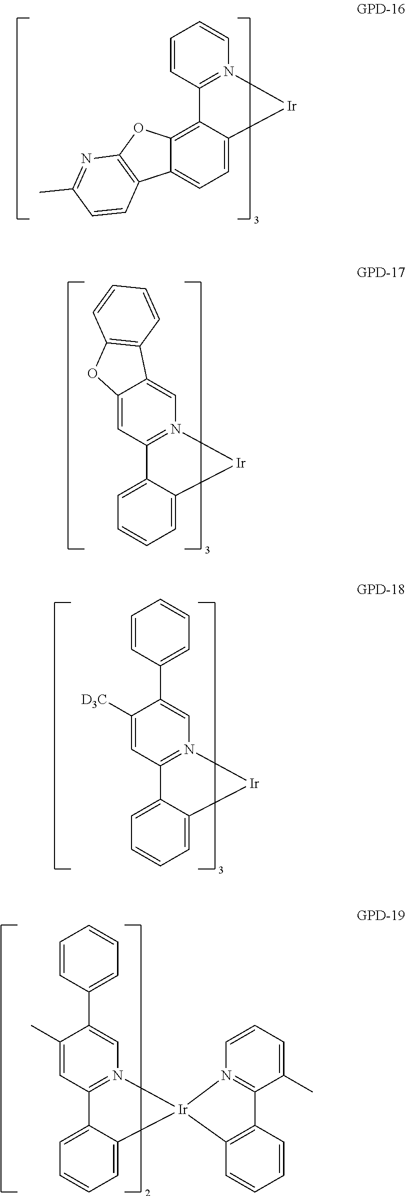

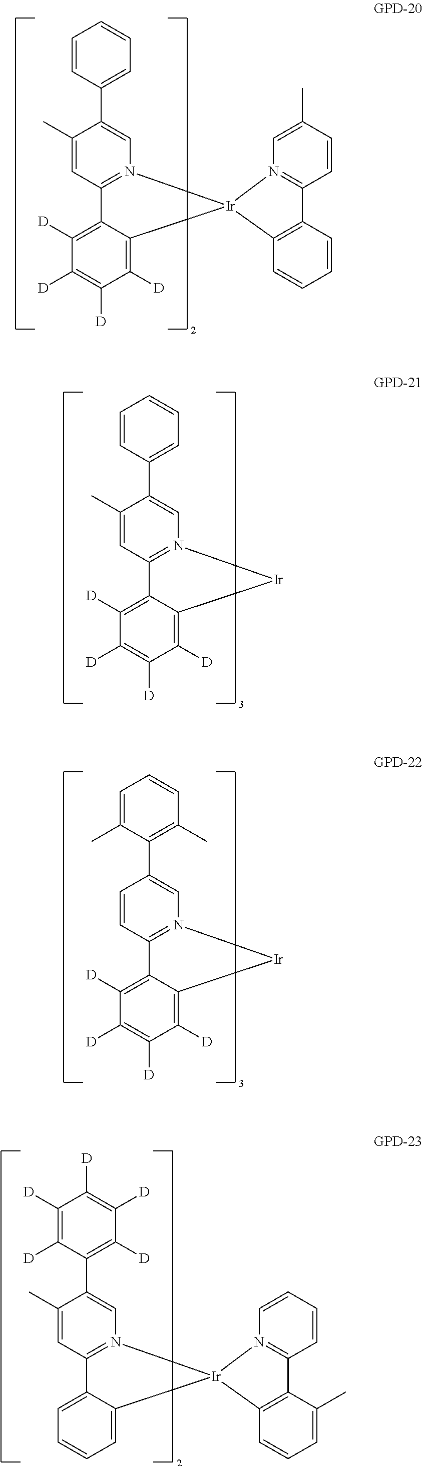

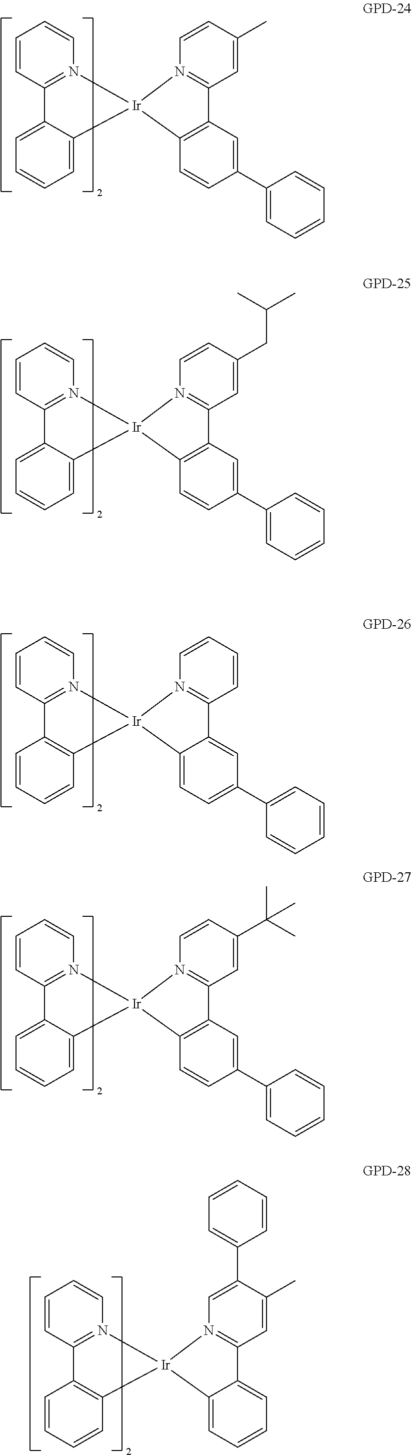

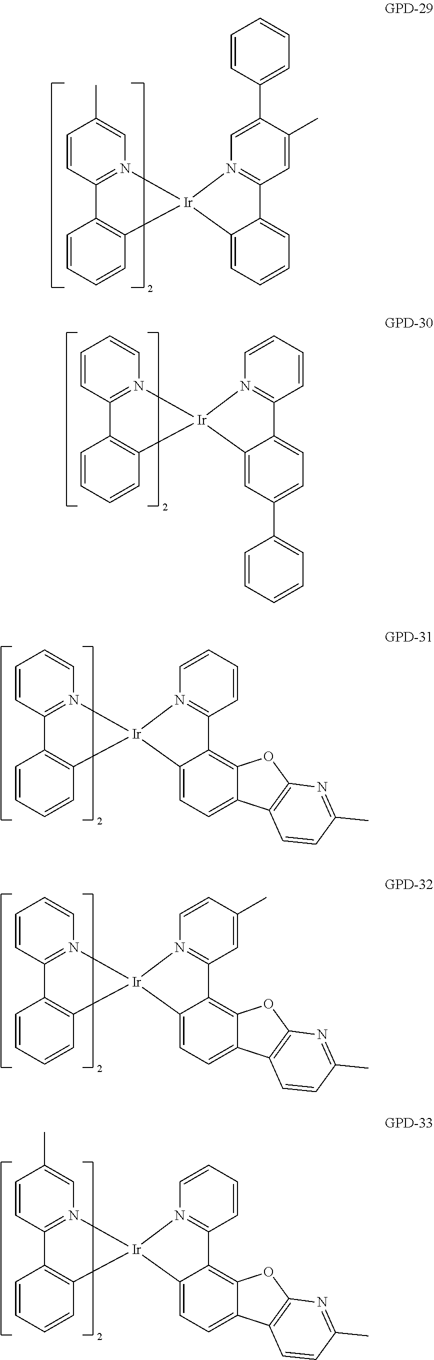

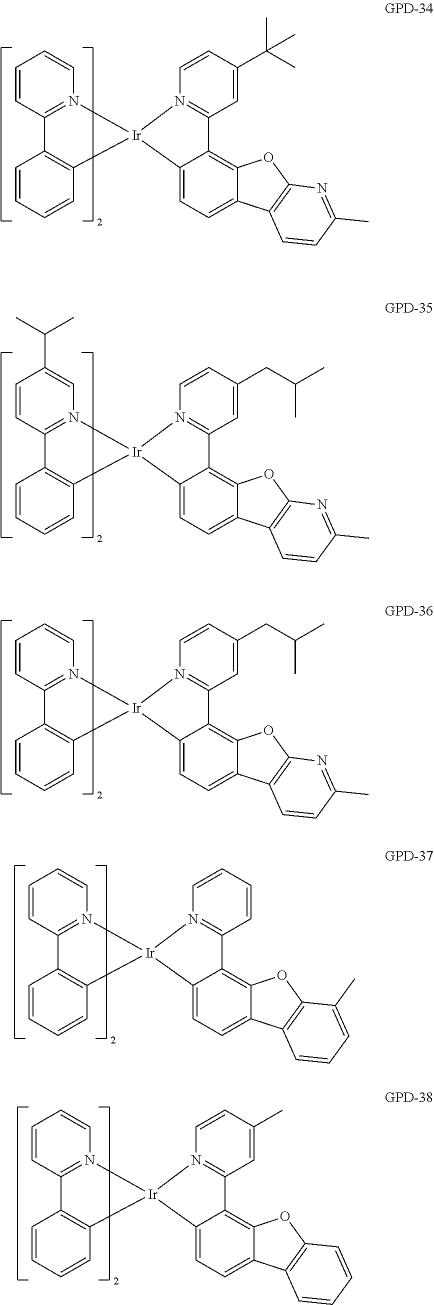

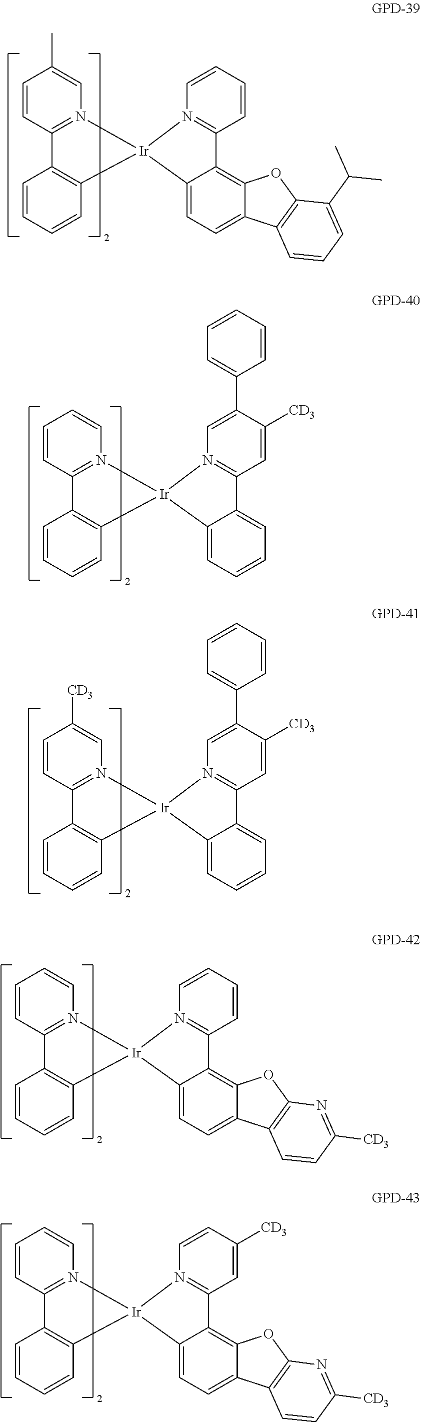

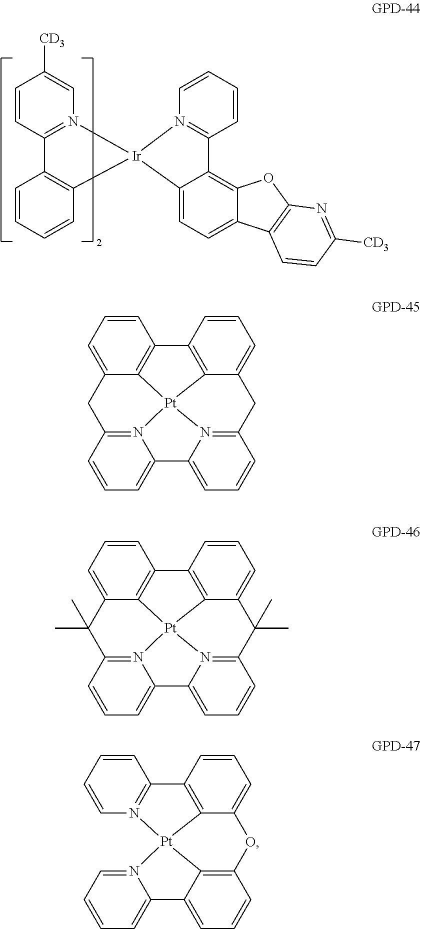

where, D is deuterium.

In one aspect of the present invention, the phosphorescent electroluminescent technology is used in the luminescent layer. The fluorescent dopant of the luminescent layer may be selected from, but not limited to one or more combinations listed in RPD-1 to RPD-28.

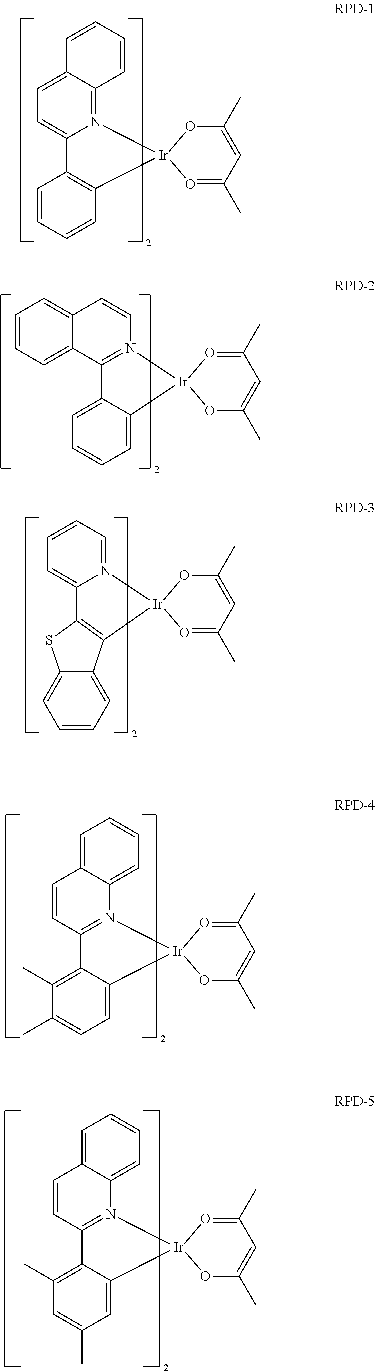

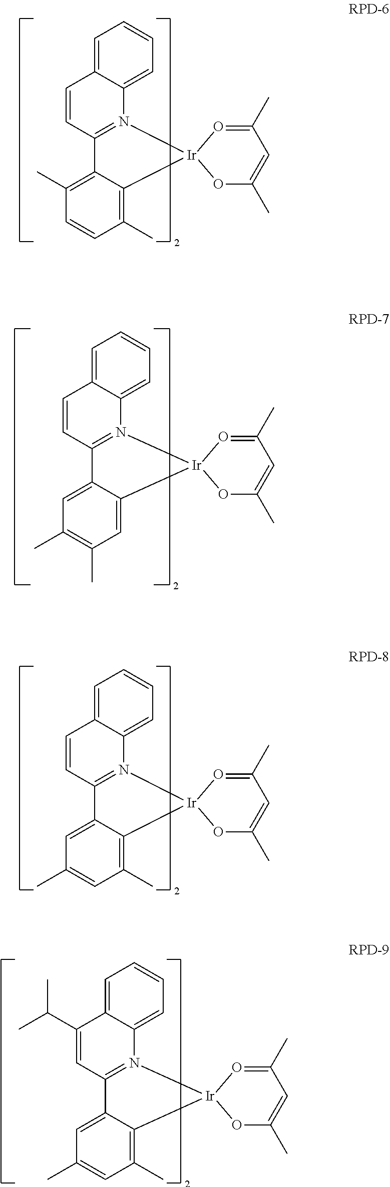

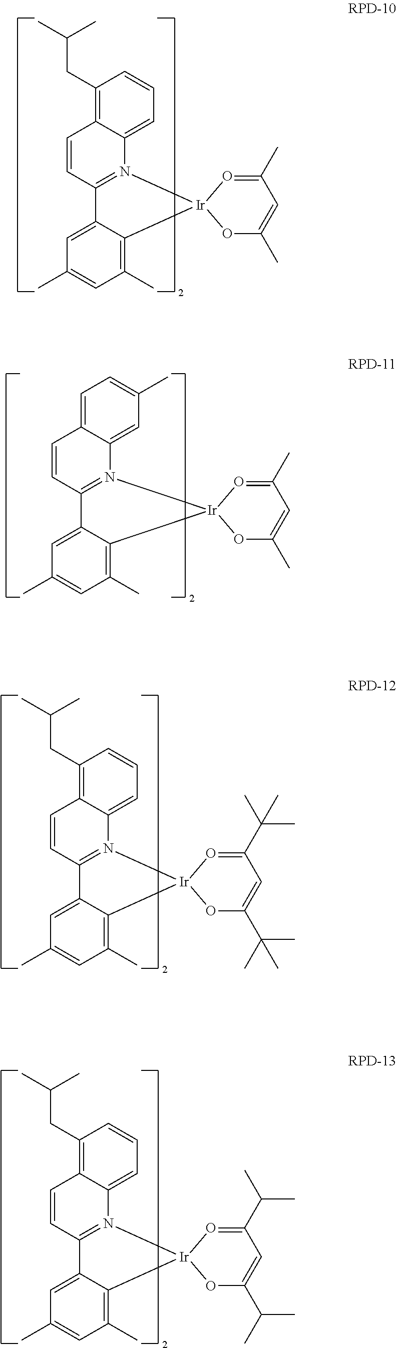

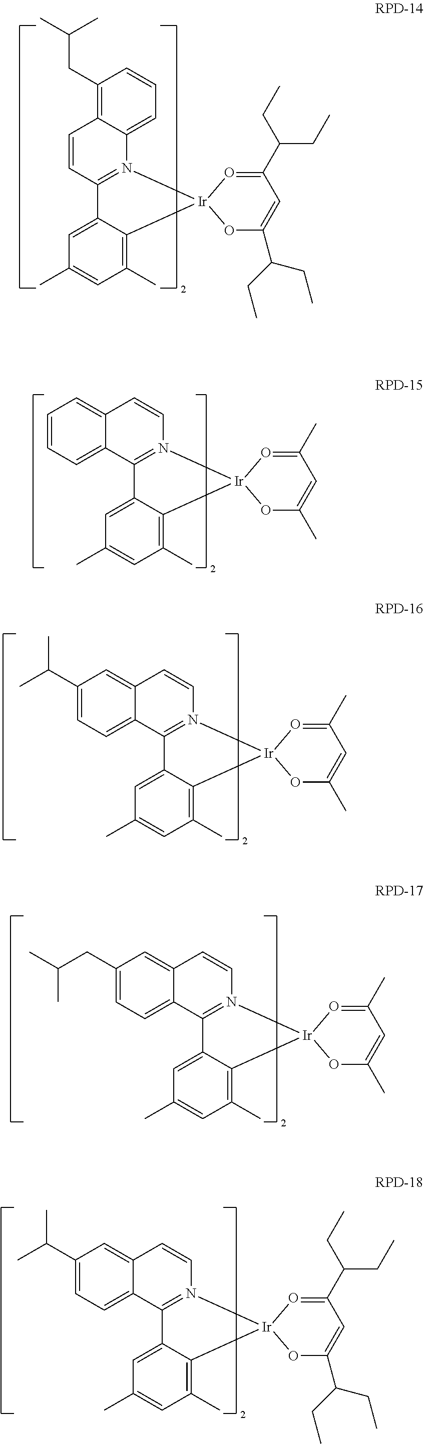

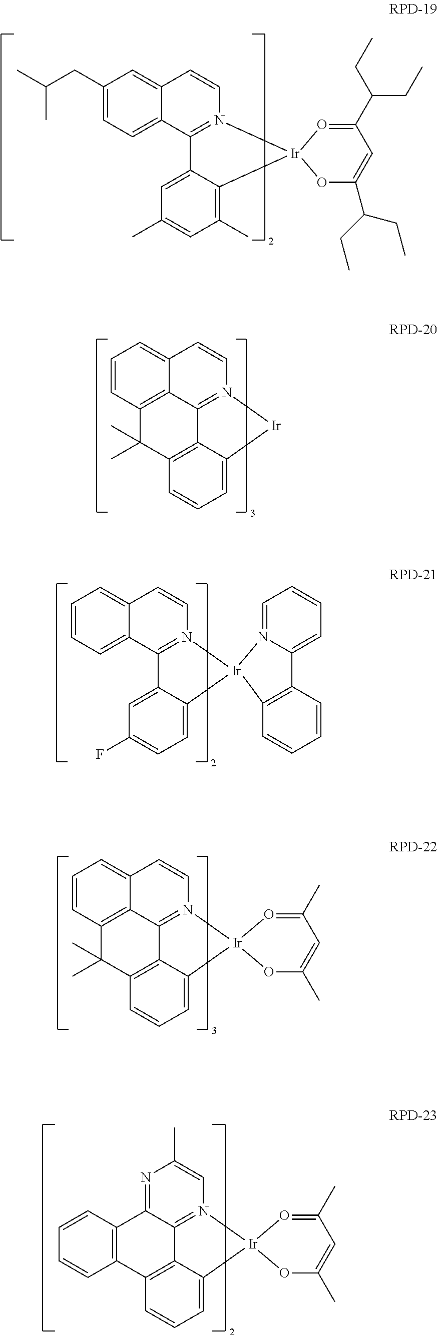

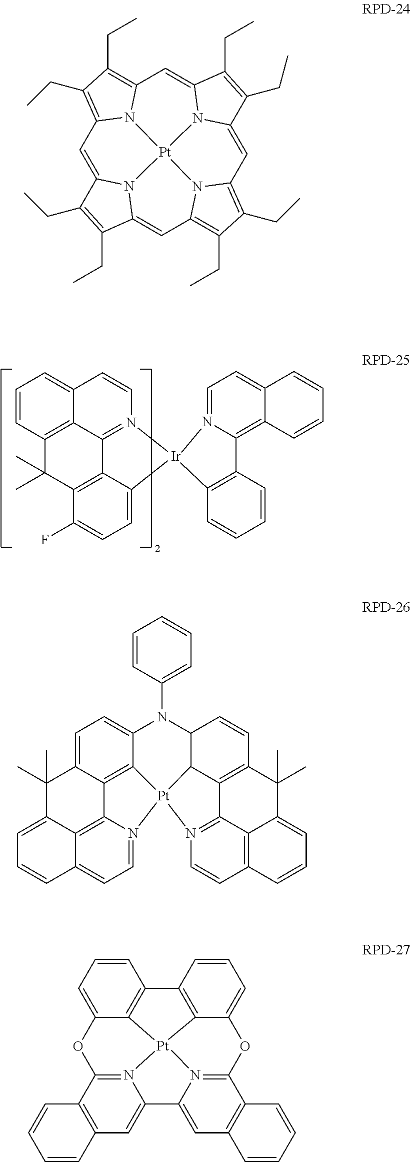

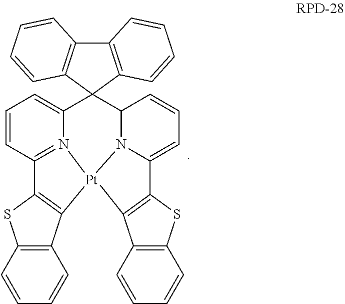

In one aspect of the preset invention, the phosphorescent electroluminescent technology is used in the luminescent layer. The fluorescent dopant of the luminescent layer may be selected from, but not limited to one or more combinations listed in YPD-1 to YPD-11.

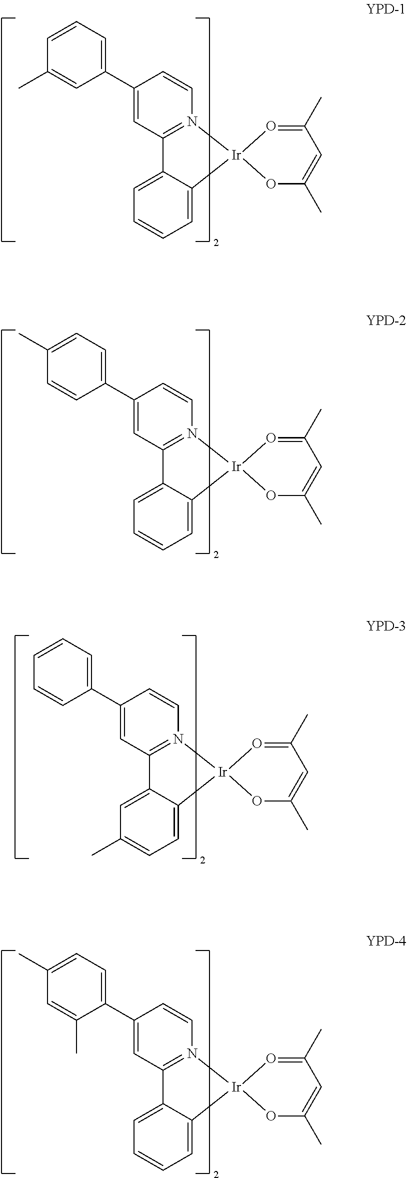

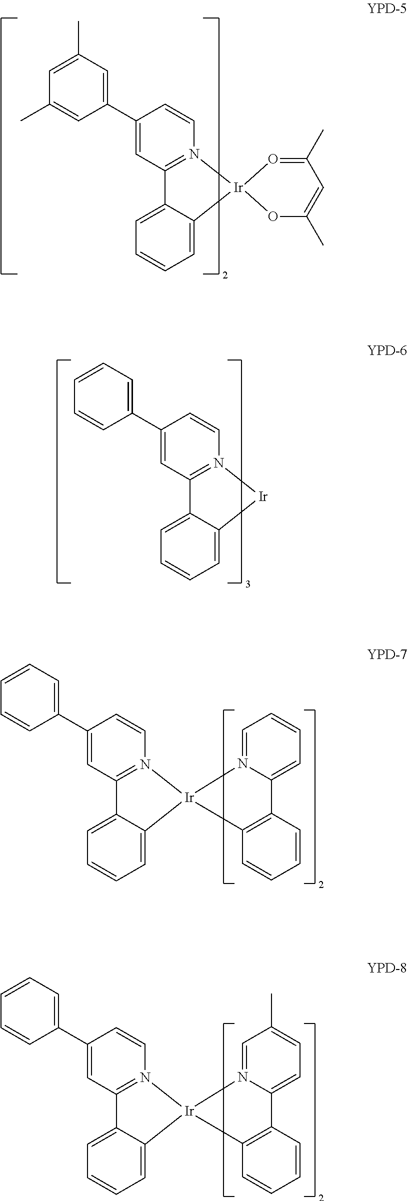

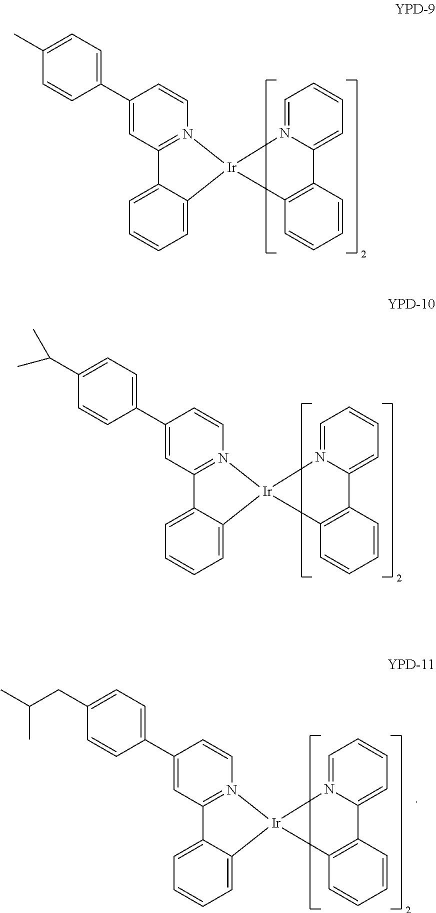

In one aspect of the present invention, the TADF luminescent technology is used in the luminescent layer. The fluorescent dopant of the luminescent layer may be selected from, but not limited to one or more combinations listed in TDE-1 to TDE-39.

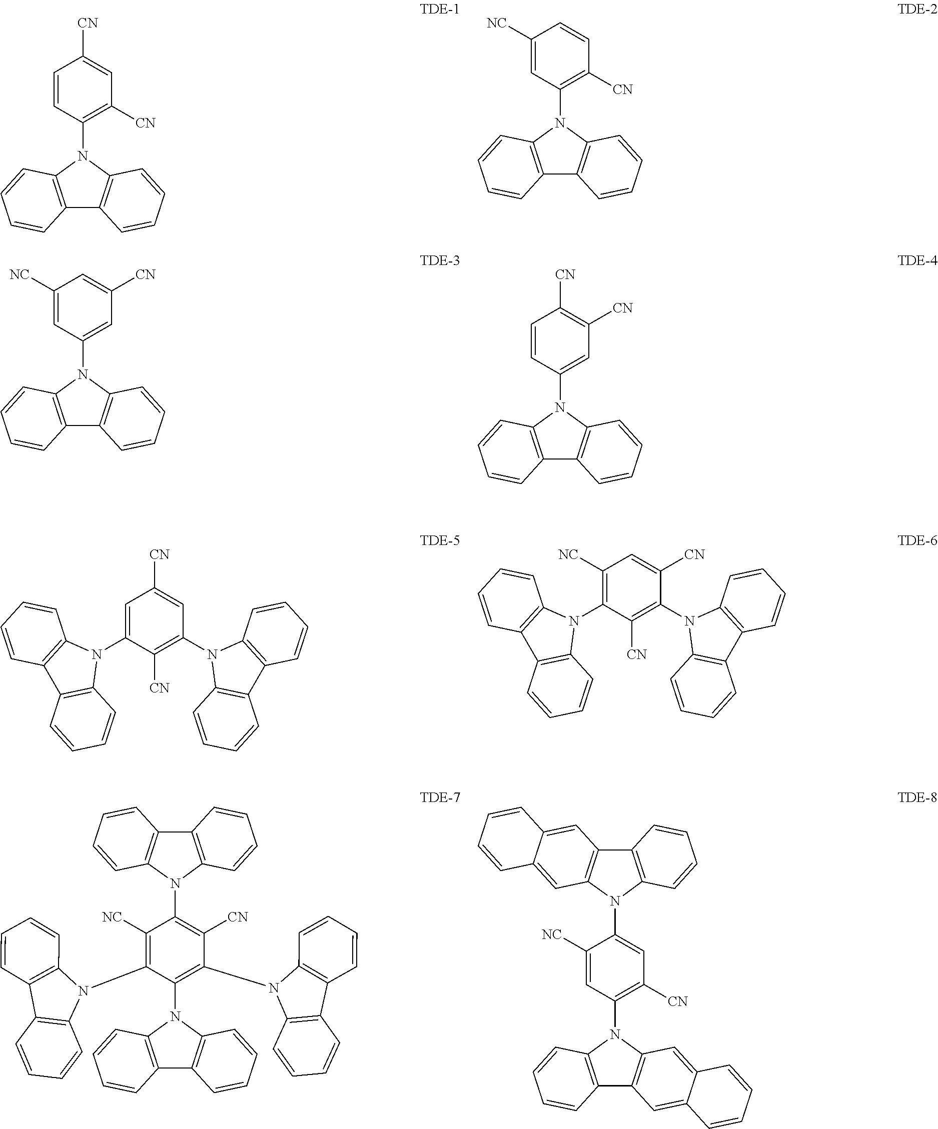

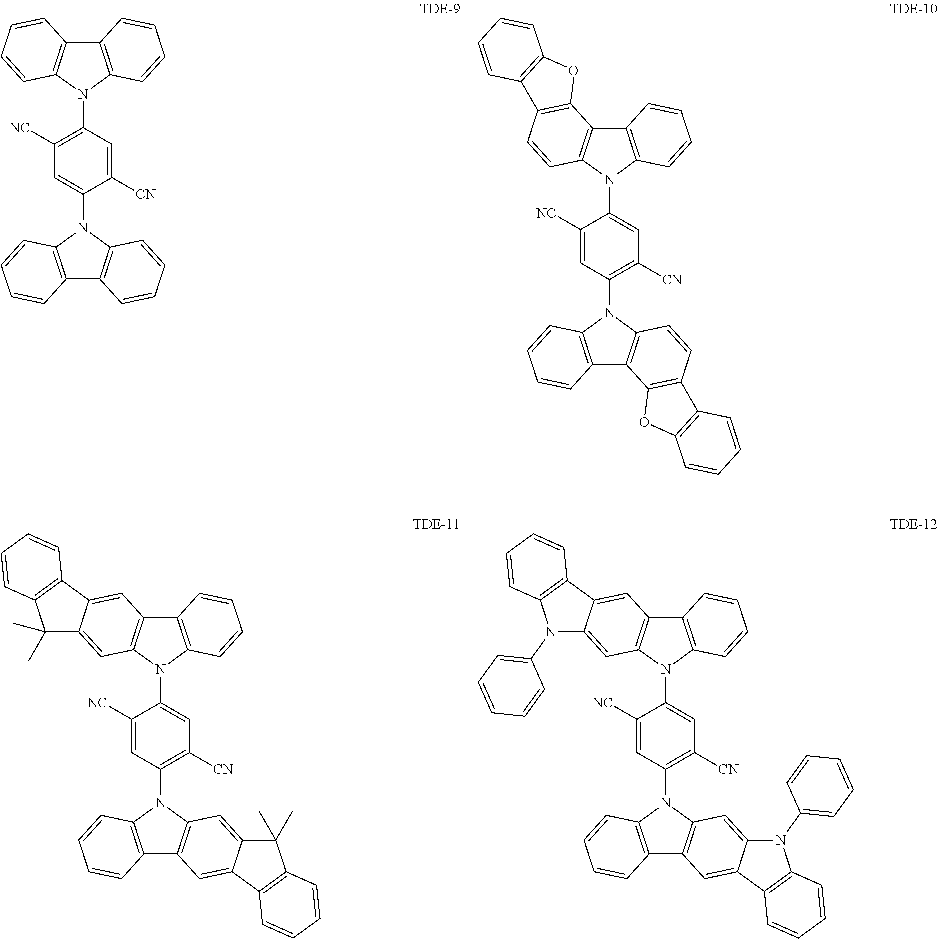 [SMILES: N#Cc1cc(-n2c3ccccc3c3ccccc32)c(C#N)cc1-n1c2ccccc2c2ccccc21 ; N#Cc1cc(-n2c3ccccc3c3c4oc5ccccc5c4ccc32)c(C#N)cc1-n1c2ccccc2c2c3oc4ccccc4c3ccc21 ; CC1(C)c2ccccc2-c2cc3c4ccccc4n(-c4cc(C#N)c(-n5c6ccccc6c6cc7c(cc65)C(C)(C)c5ccccc5-7)cc4C#N)c3cc21 ; N#Cc1cc(-n2c3ccccc3c3cc4c5ccccc5n(-c5ccccc5)c4cc32)c(C#N)cc1-n1c2ccccc2c2cc3c4ccccc4n(-c4ccccc4)c3cc21]

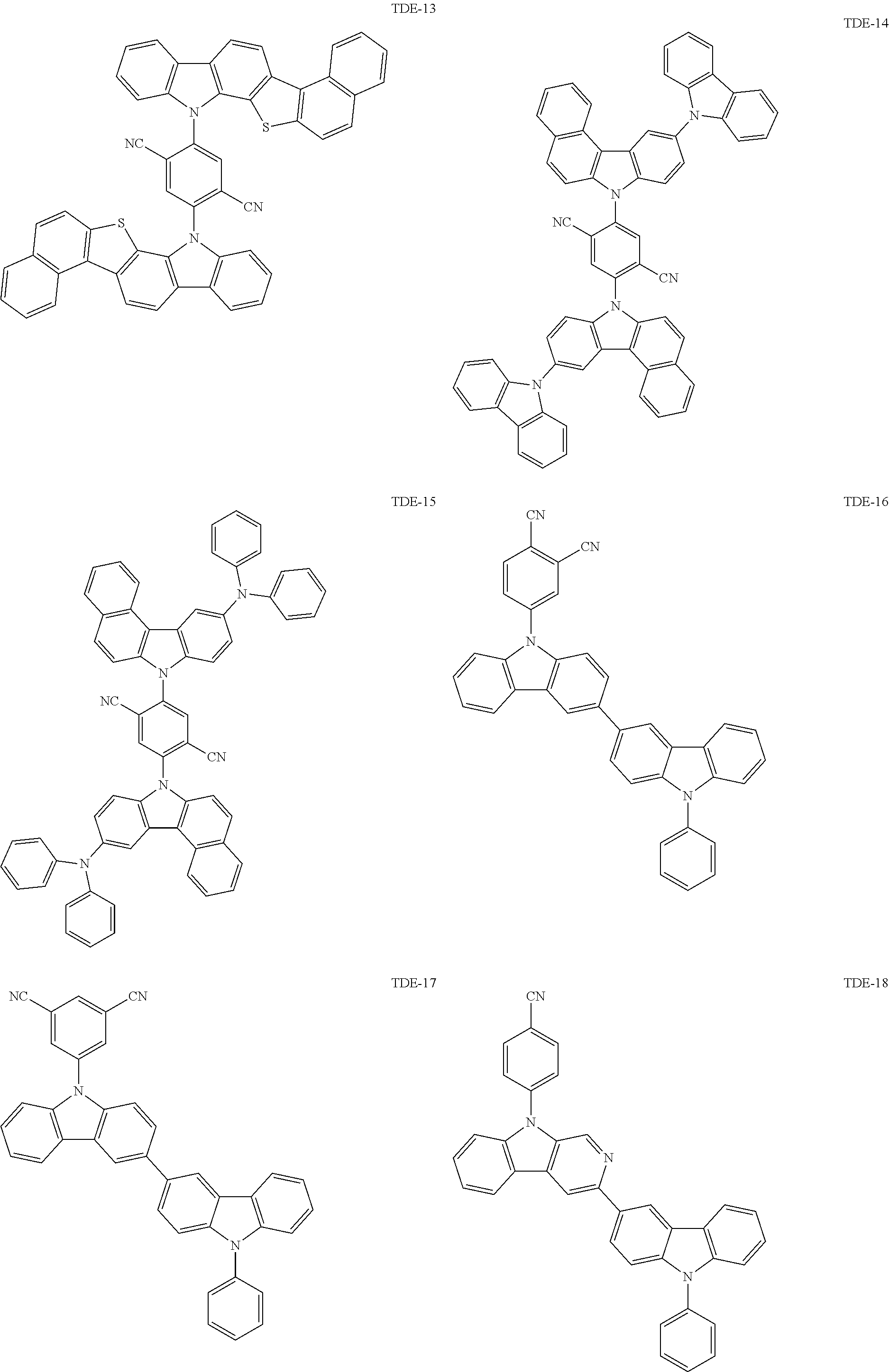

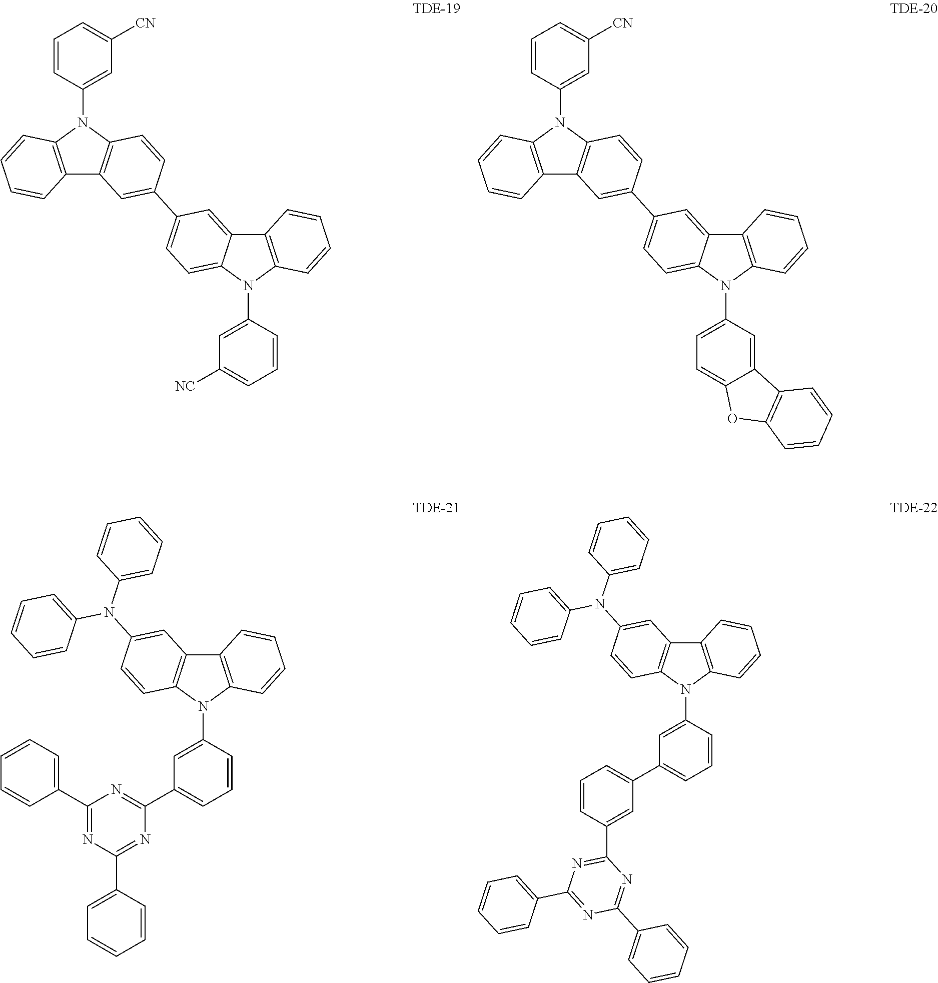 [SMILES: N#Cc1cccc(-n2c3ccccc3c3cc(-c4ccc5c(c4)c4ccccc4n5-c4cccc(C#N)c4)ccc32)c1 ; N#Cc1cccc(-n2c3ccccc3c3cc(-c4ccc5c(c4)c4ccccc4n5-c4ccc5oc6ccccc6c5c4)ccc32)c1 ; c1ccc(-c2nc(-c3ccccc3)nc(-c3cccc(-n4c5ccccc5c5cc(N(c6ccccc6)c6ccccc6)ccc54)c3)n2)cc1 ; c1ccc(-c2nc(-c3ccccc3)nc(-c3cccc(-c4cccc(-n5c6ccccc6c6cc(N(c7ccccc7)c7ccccc7)ccc65)c4)c3)n2)cc1]

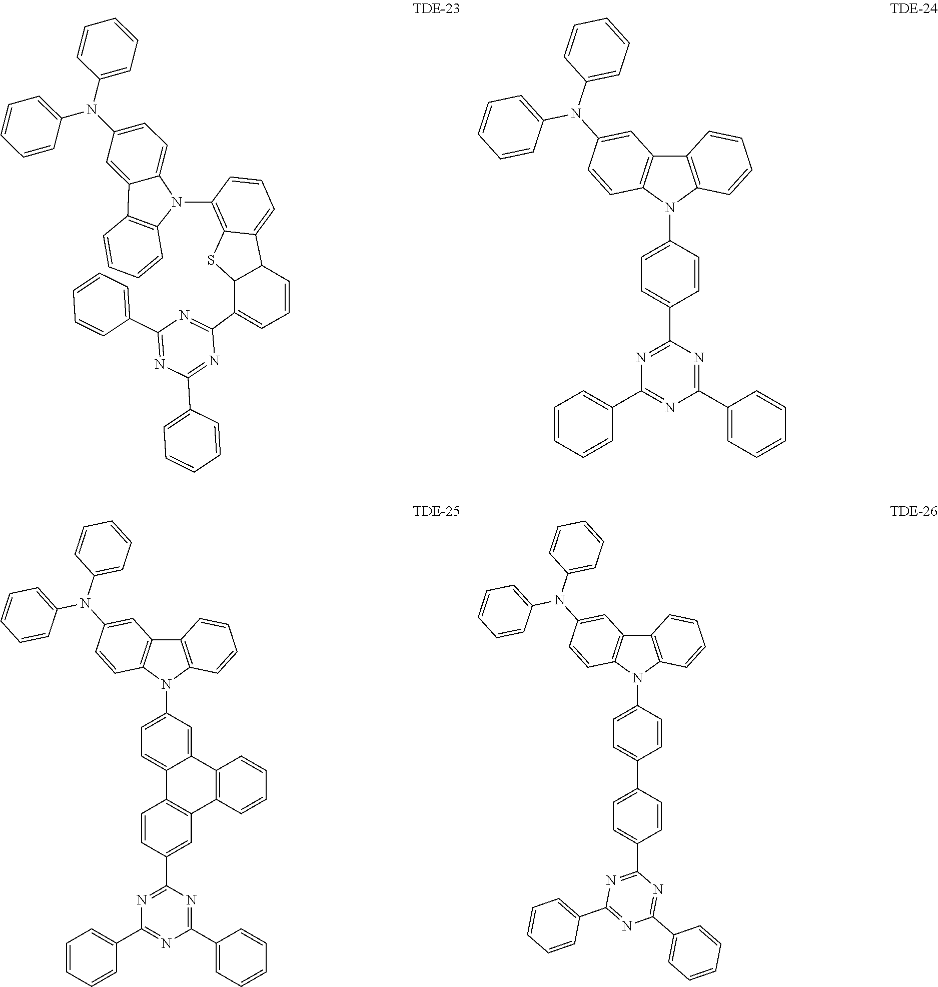 [SMILES: C1=CC2c3cccc(-n4c5ccccc5c5cc(N(c6ccccc6)c6ccccc6)ccc54)c3SC2C(c2nc(-c3ccccc3)nc(-c3ccccc3)n2)=C1 ; c1ccc(-c2nc(-c3ccccc3)nc(-c3ccc(-n4c5ccccc5c5cc(N(c6ccccc6)c6ccccc6)ccc54)cc3)n2)cc1 ; c1ccc(-c2nc(-c3ccccc3)nc(-c3ccc4c5ccc(-n6c7ccccc7c7cc(N(c8ccccc8)c8ccccc8)ccc76)cc5c5ccccc5c4c3)n2)cc1 ; c1ccc(-c2nc(-c3ccccc3)nc(-c3ccc(-c4ccc(-n5c6ccccc6c6cc(N(c7ccccc7)c7ccccc7)ccc65)cc4)cc3)n2)cc1]

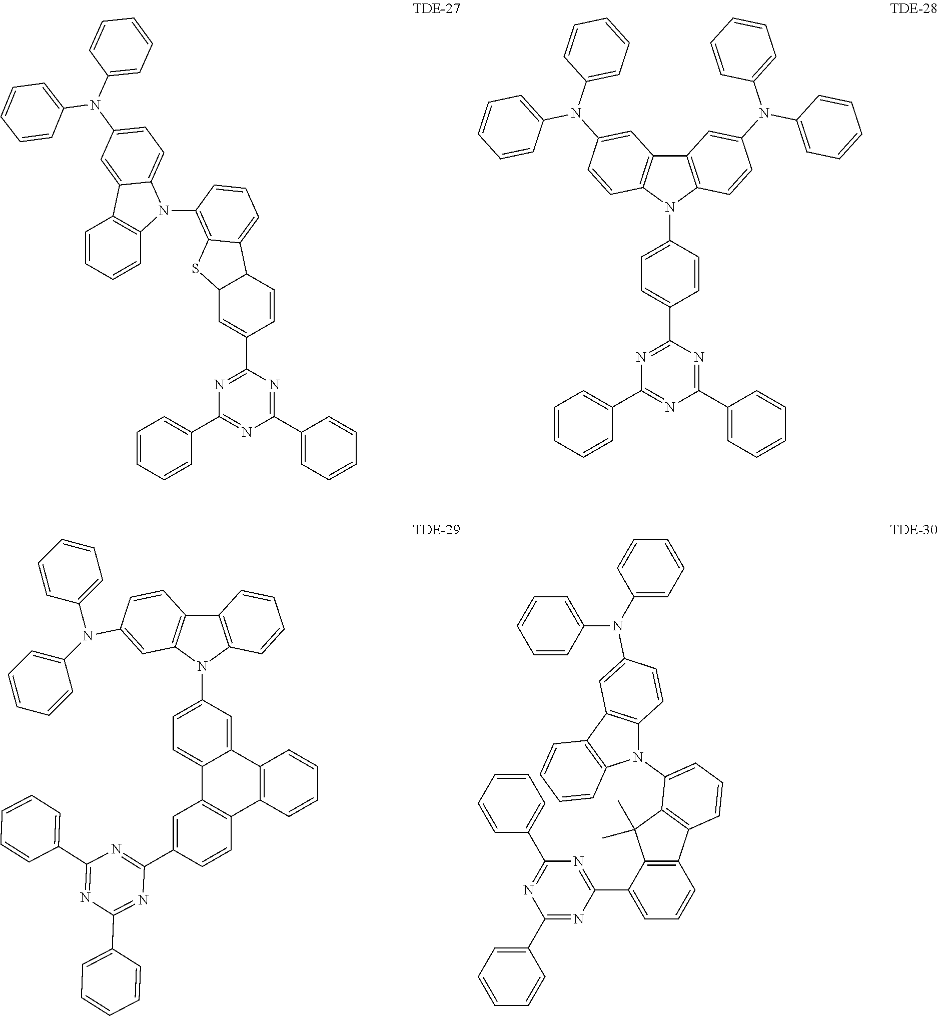 [SMILES: C1=CC2c3cccc(-n4c5ccccc5c5cc(N(c6ccccc6)c6ccccc6)ccc54)c3SC2C=C1c1nc(-c2ccccc2)nc(-c2ccccc2)n1 ; c1ccc(-c2nc(-c3ccccc3)nc(-c3ccc(-n4c5ccc(N(c6ccccc6)c6ccccc6)cc5c5cc(N(c6ccccc6)c6ccccc6)ccc54)cc3)n2)cc1 ; c1ccc(-c2nc(-c3ccccc3)nc(-c3ccc4c5ccccc5c5cc(-n6c7ccccc7c7ccc(N(c8ccccc8)c8ccccc8)cc76)ccc5c4c3)n2)cc1 ; CC1(C)c2c(-c3nc(-c4ccccc4)nc(-c4ccccc4)n3)cccc2-c2cccc(-n3c4ccccc4c4cc(N(c5ccccc5)c5ccccc5)ccc43)c21]

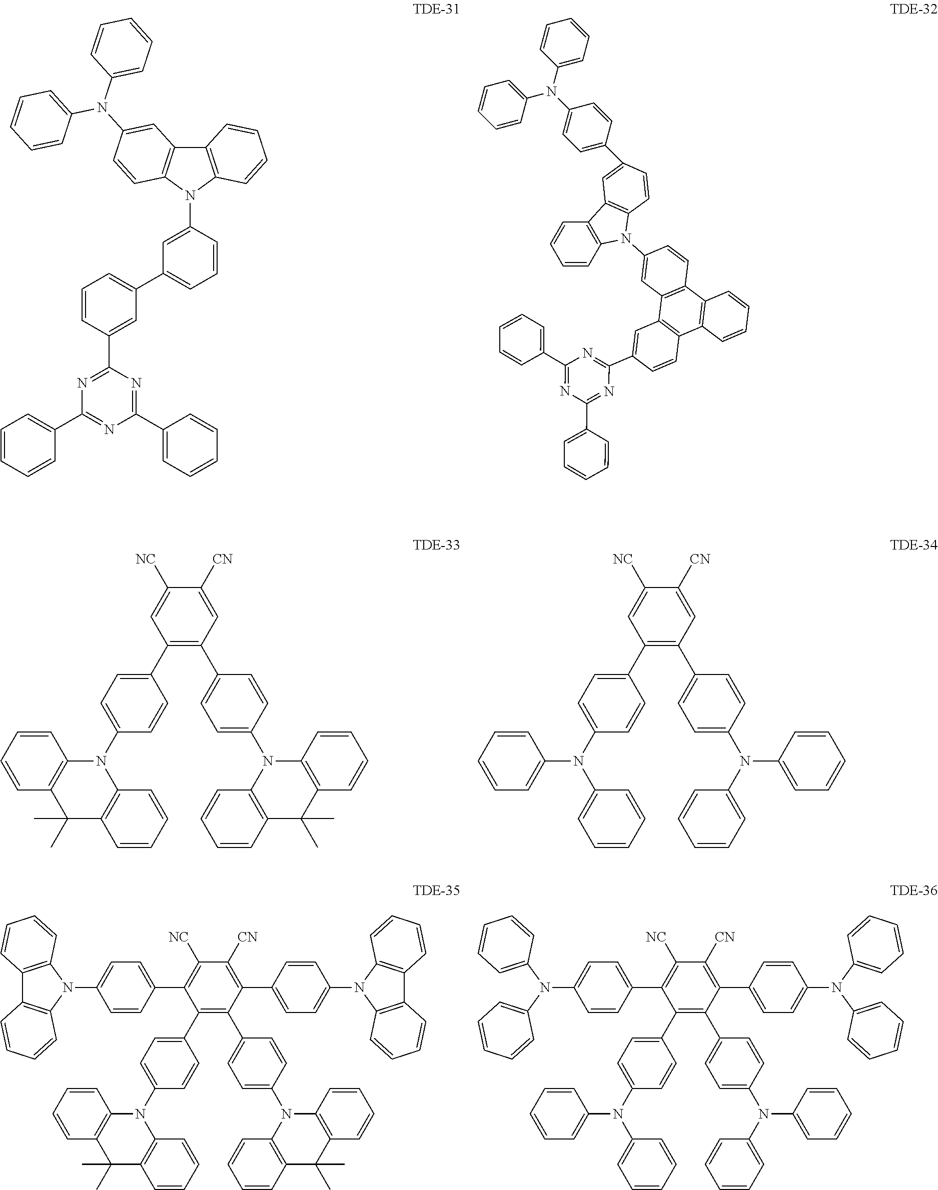

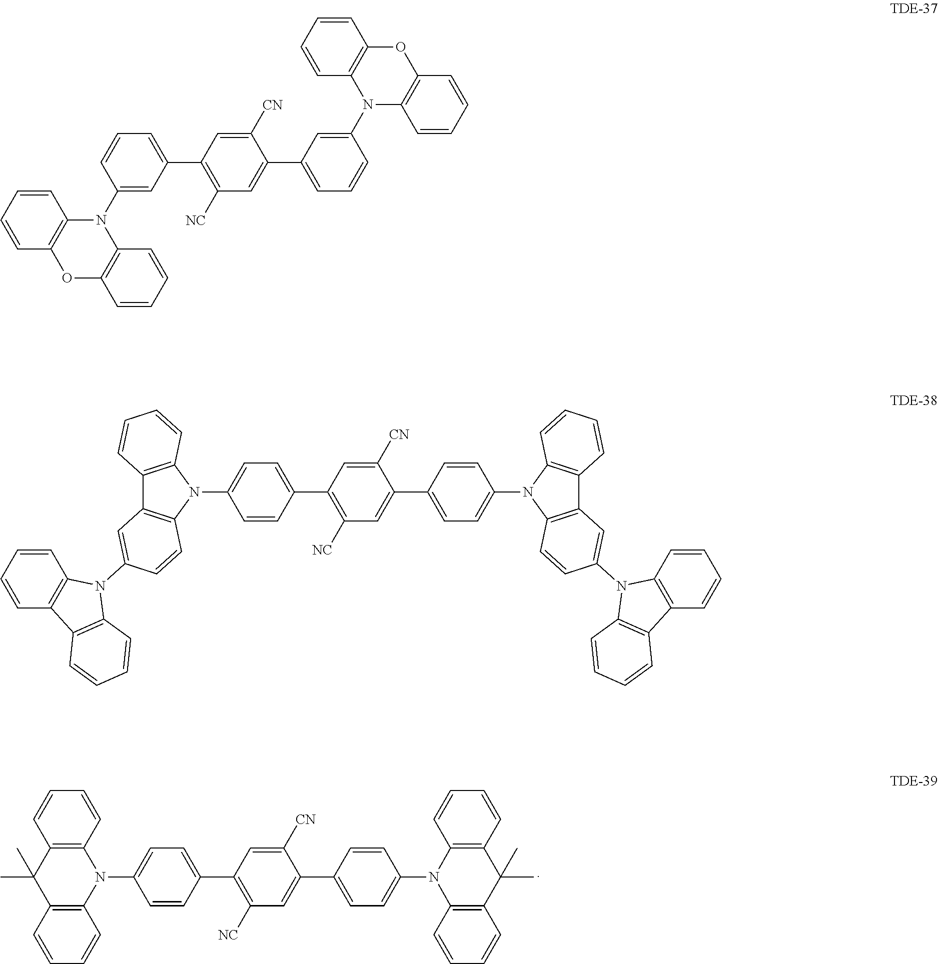

In one aspect of the present invention, the TADF luminescent technology is used in the luminescent layer. The fluorescent host material of the luminescent layer may be selected from, but not limited to one or more compounds listed in TDH1 to TDH24.

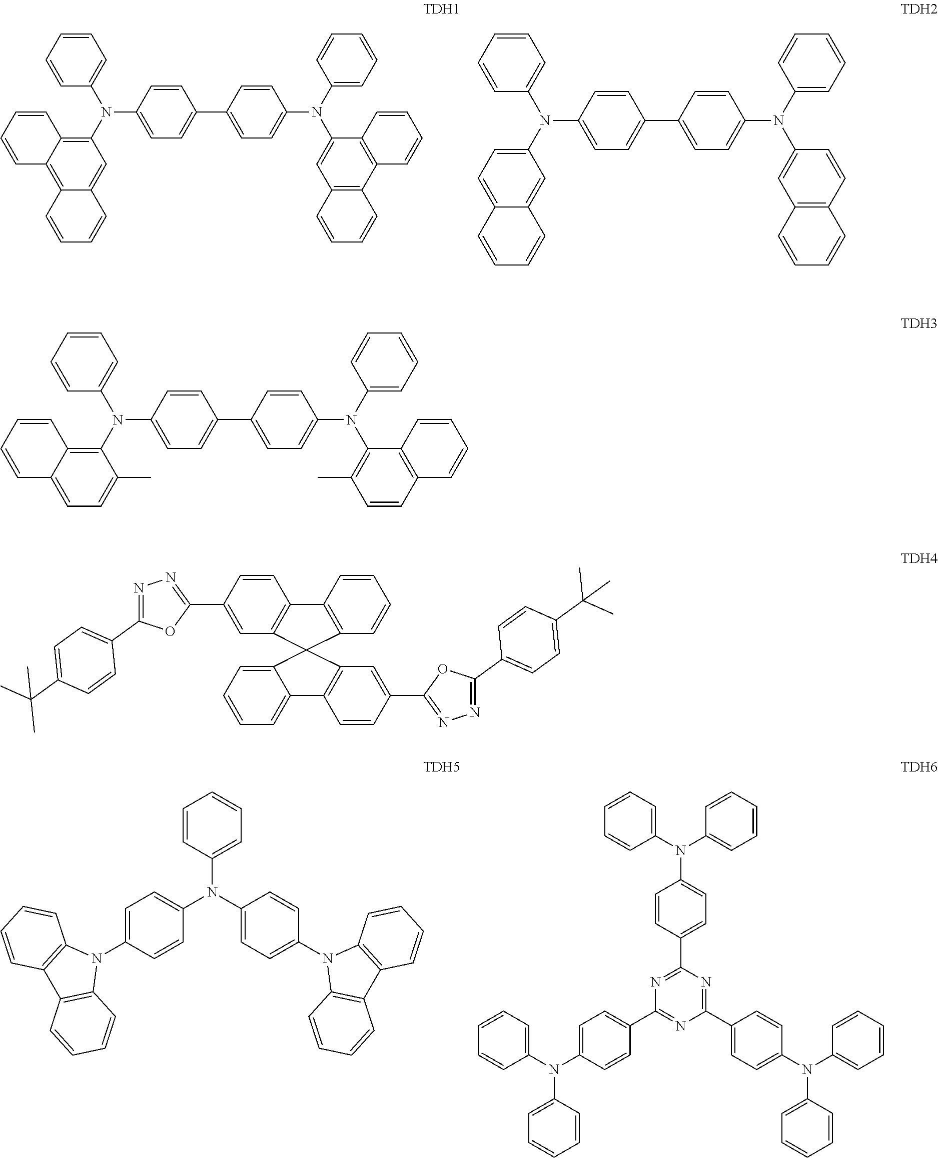

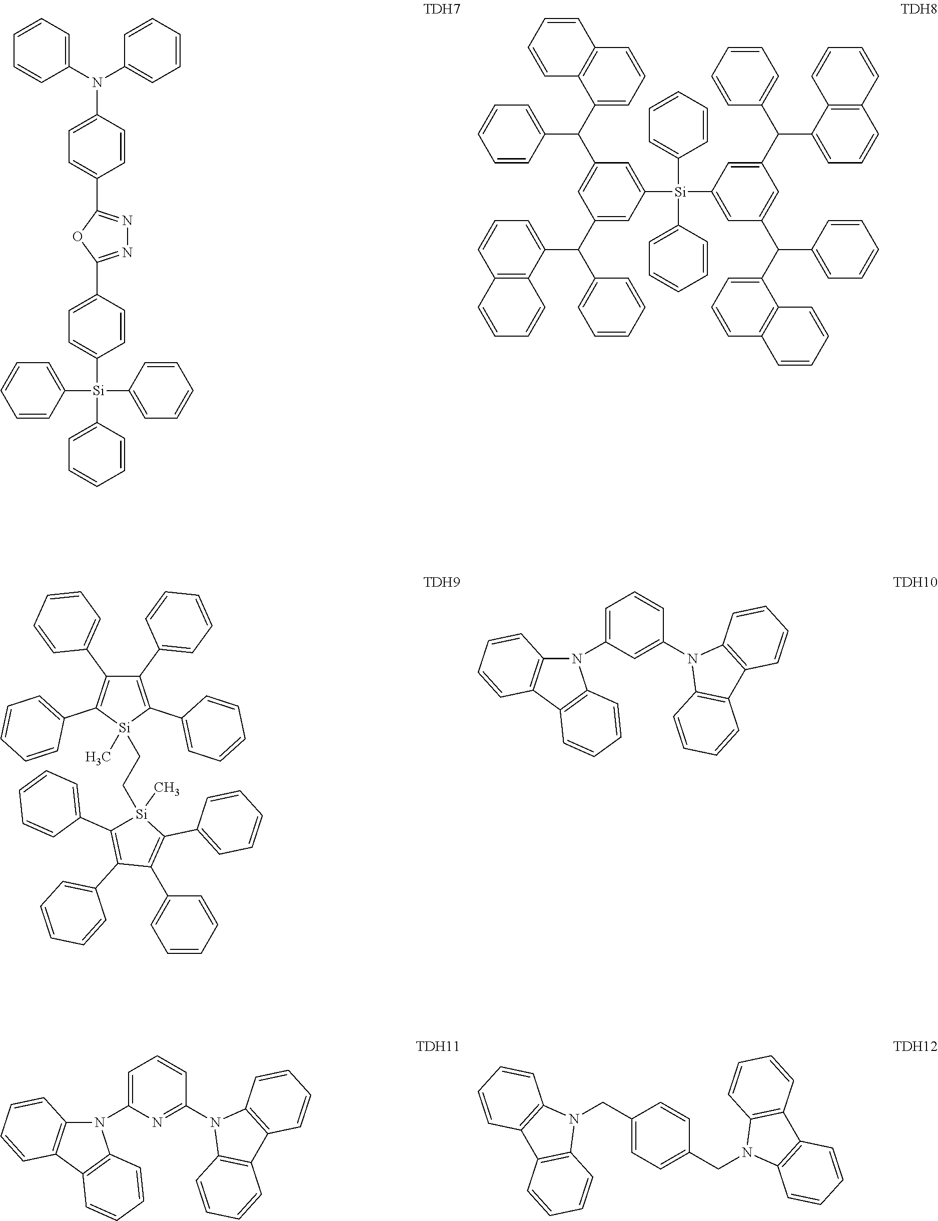

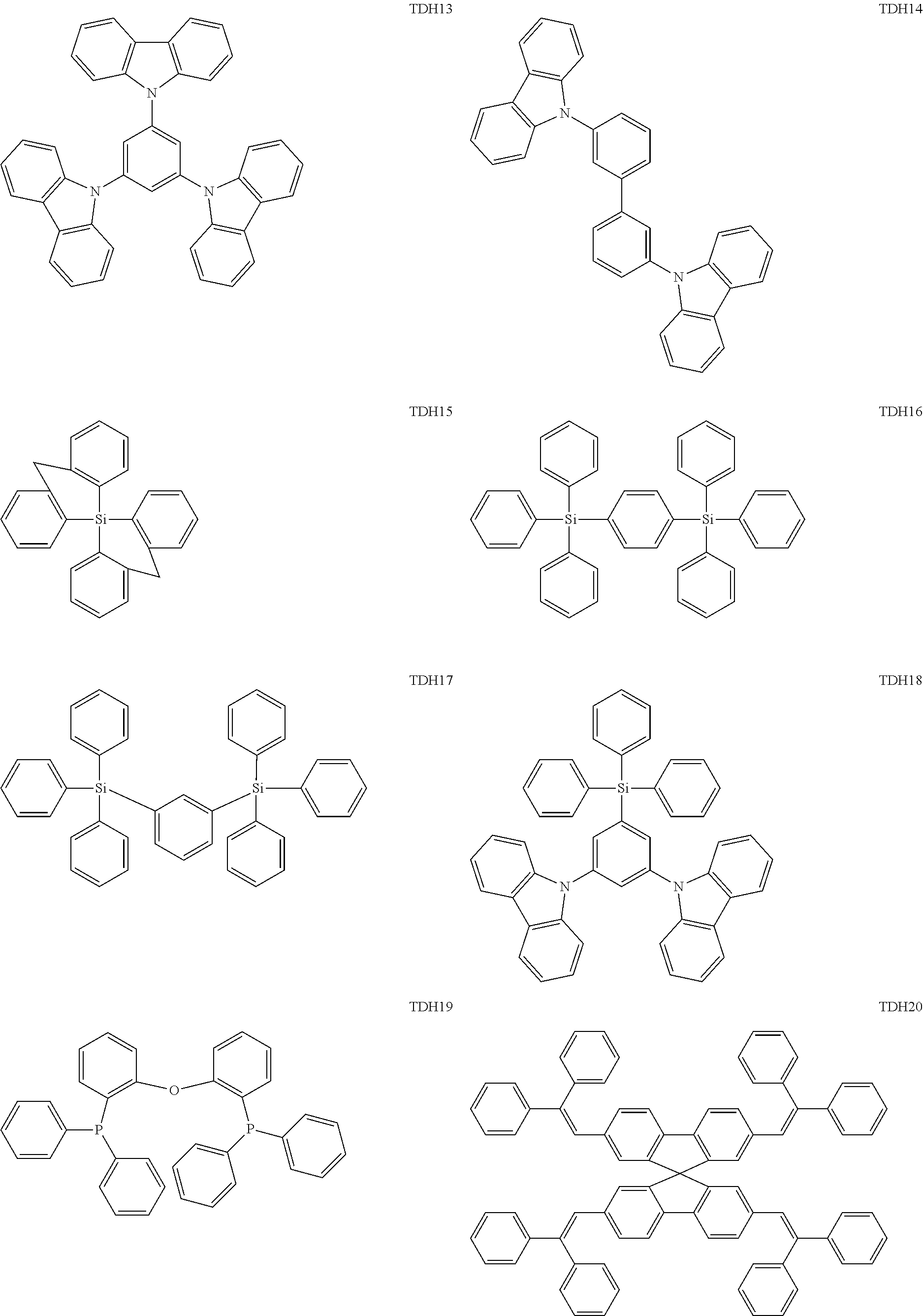

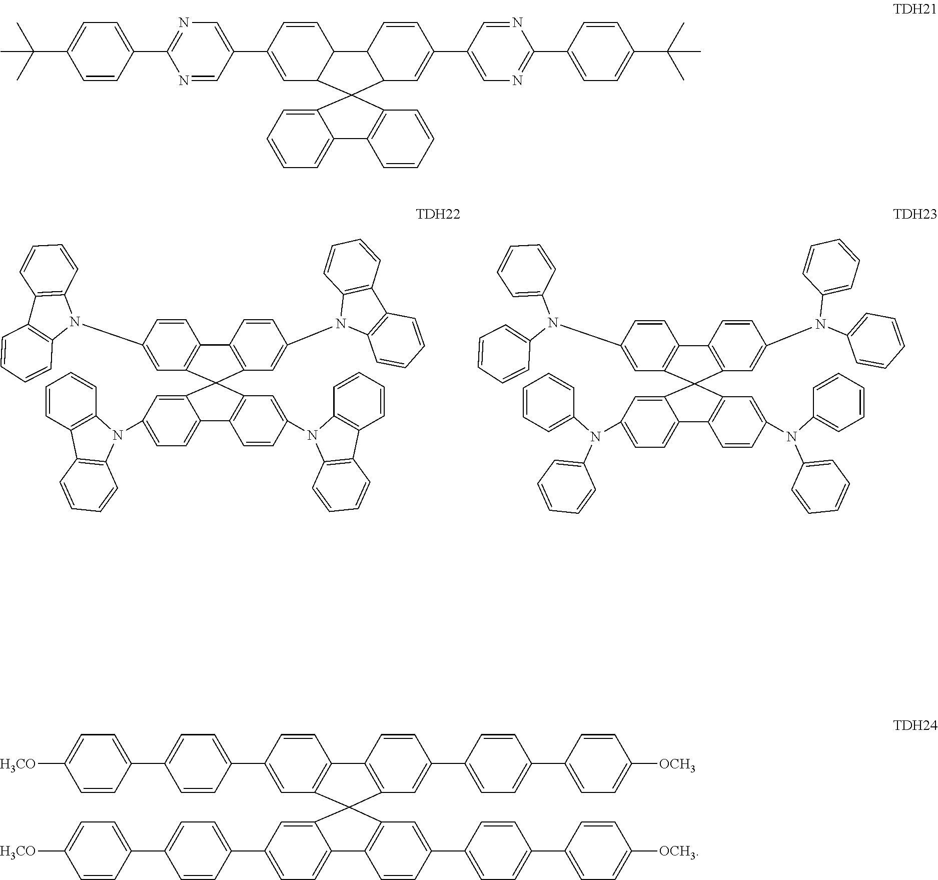

The OLED organic layer may further include an electron transport region between the luminescent layer and the cathode. The electron transport region may be an electron transport layer (ETL) with a single-layer structure, including a single-layer ETL only containing a compound and a single-layer ETL containing a plurality of compounds. The electron transport region also may be a multilayered structure including at least one of an electron injection layer (EIL), an electron transport layer (ETL) and an electron blocking layer (EBL).

In one aspect of the present invention, the electron transport layer material may be selected from, but not limited to one or more combinations listed in ET-1 to ET-57.

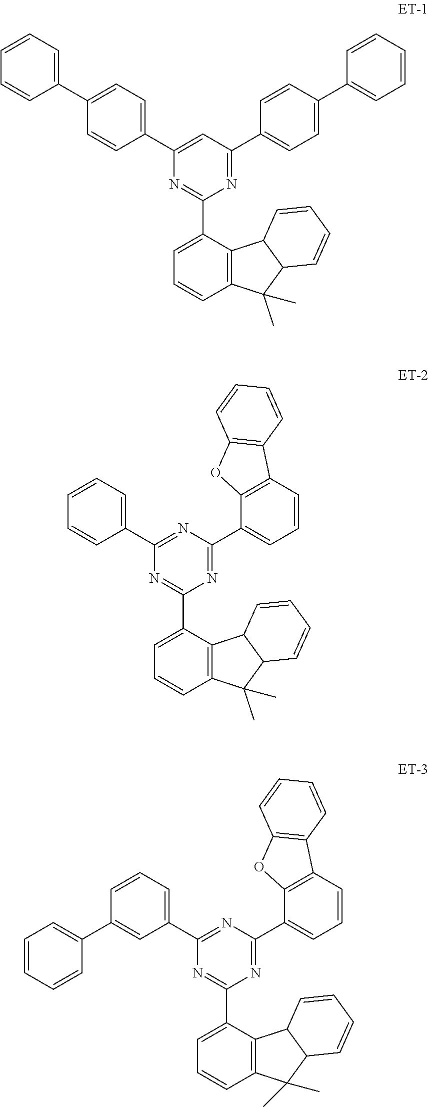 [SMILES: CC1(C)c2cccc(-c3nc(-c4ccc(-c5ccccc5)cc4)cc(-c4ccc(-c5ccccc5)cc4)n3)c2C2C=CC=CC21 ; CC1(C)c2cccc(-c3nc(-c4ccccc4)nc(-c4cccc5c4oc4ccccc45)n3)c2C2C=CC=CC21 ; CC1(C)c2cccc(-c3nc(-c4cccc(-c5ccccc5)c4)nc(-c4cccc5c4oc4ccccc45)n3)c2C2C=CC=CC21]

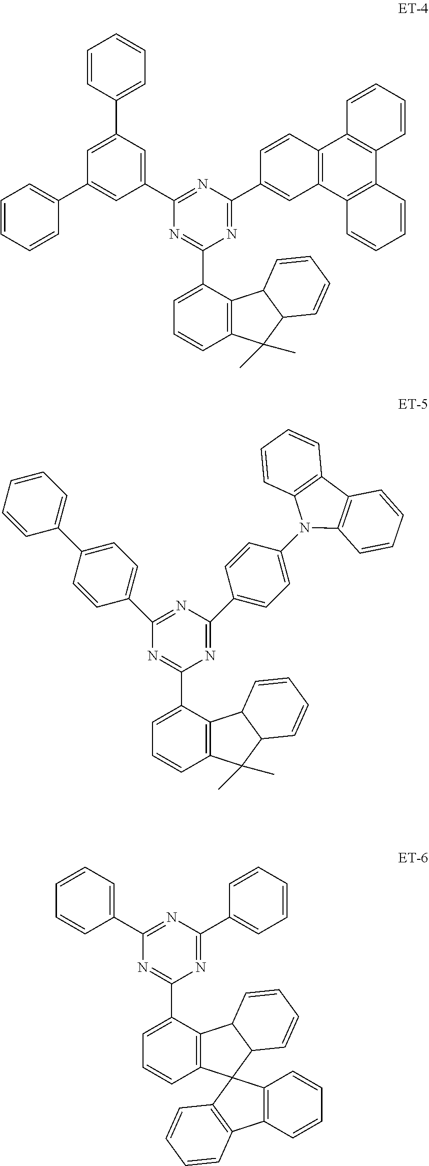 [SMILES: CC1(C)c2cccc(-c3nc(-c4cc(-c5ccccc5)cc(-c5ccccc5)c4)nc(-c4ccc5c6ccccc6c6ccccc6c5c4)n3)c2C2C=CC=CC21 ; CC1(C)c2cccc(-c3nc(-c4ccc(-c5ccccc5)cc4)nc(-c4ccc(-n5c6ccccc6c6ccccc65)cc4)n3)c2C2C=CC=CC21 ; C1=CC2c3c(-c4nc(-c5ccccc5)nc(-c5ccccc5)n4)cccc3C3(c4ccccc4-c4ccccc43)C2C=C1]

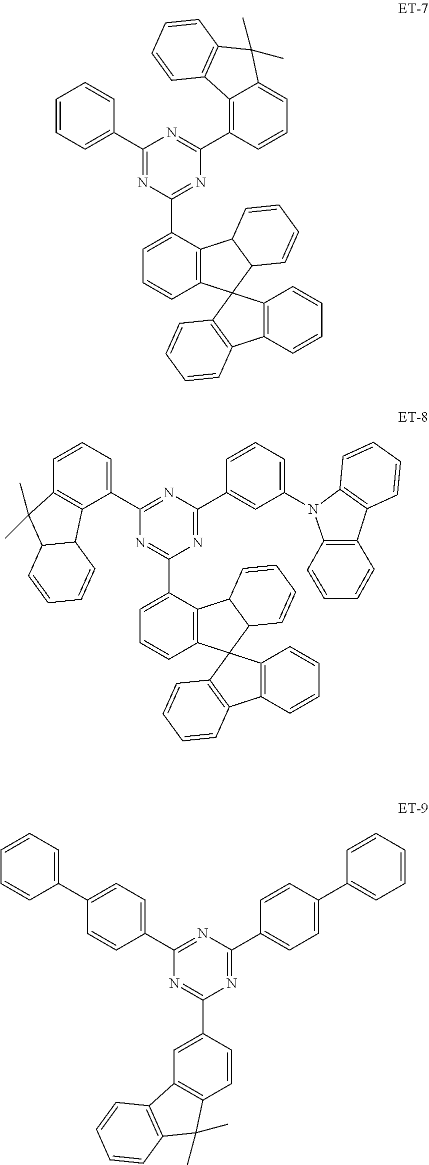 [SMILES: CC1(C)c2ccccc2-c2c(-c3nc(-c4ccccc4)nc(-c4cccc5c4C4C=CC=CC4C54c5ccccc5-c5ccccc54)n3)cccc21 ; CC1(C)c2cccc(-c3nc(-c4cccc(-n5c6ccccc6c6ccccc65)c4)nc(-c4cccc5c4C4C=CC=CC4C54c5ccccc5-c5ccccc54)n3)c2C2C=CC=CC21 ; CC1(C)c2ccccc2-c2cc(-c3nc(-c4ccc(-c5ccccc5)cc4)nc(-c4ccc(-c5ccccc5)cc4)n3)ccc21]

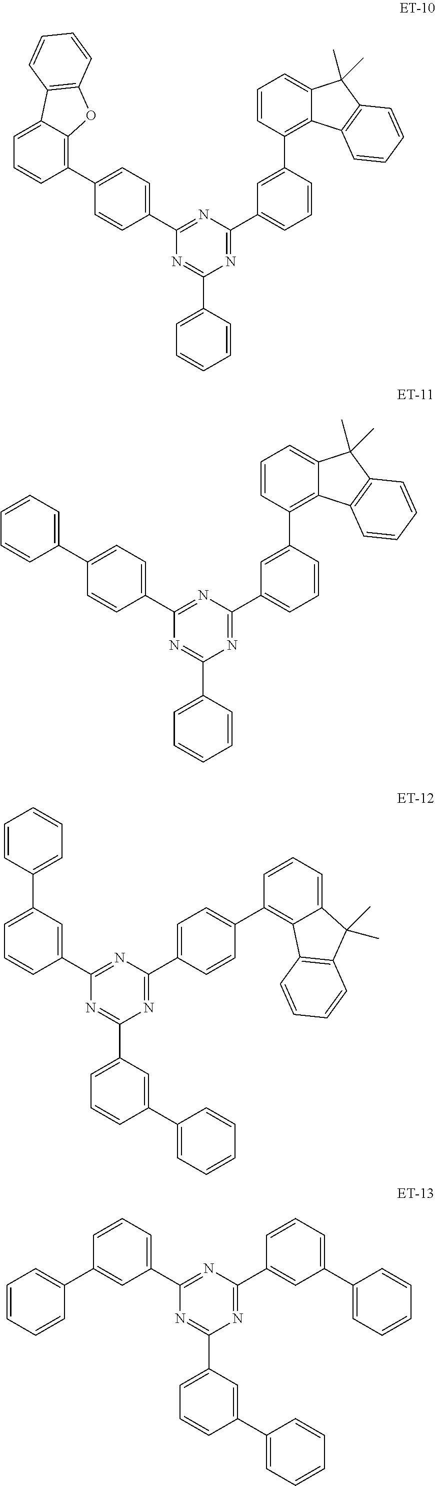 [SMILES: CC1(C)c2ccccc2-c2c(-c3cccc(-c4nc(-c5ccccc5)nc(-c5ccc(-c6cccc7c6oc6ccccc67)cc5)n4)c3)cccc21 ; CC1(C)c2ccccc2-c2c(-c3cccc(-c4nc(-c5ccccc5)nc(-c5ccc(-c6ccccc6)cc5)n4)c3)cccc21 ; CC1(C)c2ccccc2-c2c(-c3ccc(-c4nc(-c5cccc(-c6ccccc6)c5)nc(-c5cccc(-c6ccccc6)c5)n4)cc3)cccc21 ; c1ccc(-c2cccc(-c3nc(-c4cccc(-c5ccccc5)c4)nc(-c4cccc(-c5ccccc5)c4)n3)c2)cc1]

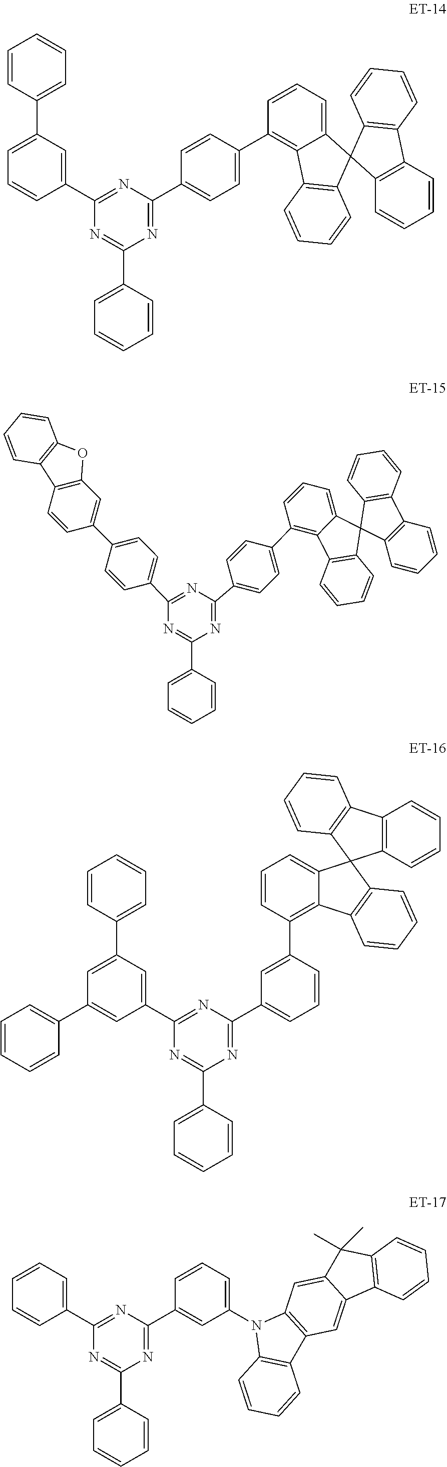 [SMILES: c1ccc(-c2cccc(-c3nc(-c4ccccc4)nc(-c4ccc(-c5cccc6c5-c5ccccc5C65c6ccccc6-c6ccccc65)cc4)n3)c2)cc1 ; c1ccc(-c2nc(-c3ccc(-c4ccc5c(c4)oc4ccccc45)cc3)nc(-c3ccc(-c4cccc5c4-c4ccccc4C54c5ccccc5-c5ccccc54)cc3)n2)cc1 ; c1ccc(-c2cc(-c3ccccc3)cc(-c3nc(-c4ccccc4)nc(-c4cccc(-c5cccc6c5-c5ccccc5C65c6ccccc6-c6ccccc65)c4)n3)c2)cc1 ; CC1(C)c2ccccc2-c2cc3c4ccccc4n(-c4cccc(-c5nc(-c6ccccc6)nc(-c6ccccc6)n5)c4)c3cc21]

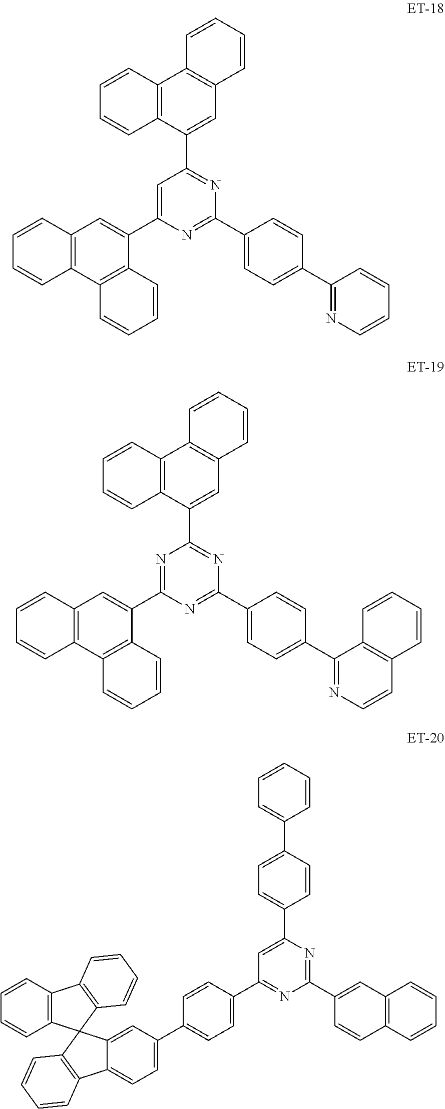 [SMILES: c1ccc(-c2ccc(-c3nc(-c4cc5ccccc5c5ccccc45)cc(-c4cc5ccccc5c5ccccc45)n3)cc2)nc1 ; c1ccc2c(-c3ccc(-c4nc(-c5cc6ccccc6c6ccccc56)nc(-c5cc6ccccc6c6ccccc56)n4)cc3)nccc2c1 ; c1ccc(-c2ccc(-c3cc(-c4ccc(-c5ccc6c(c5)C5(c7ccccc7-c7ccccc75)c5ccccc5-6)cc4)nc(-c4ccc5ccccc5c4)n3)cc2)cc1]

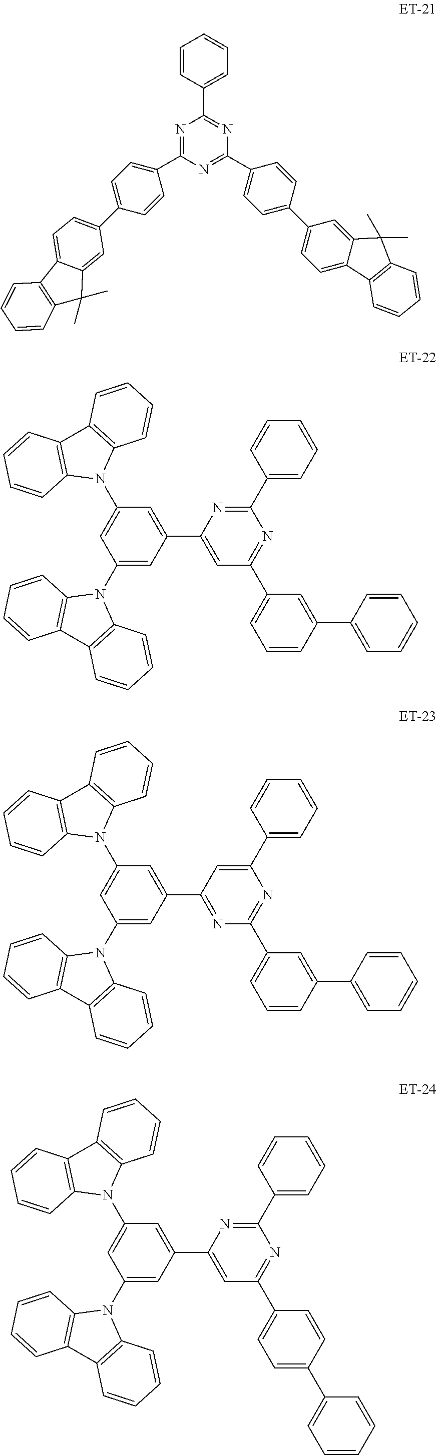 [SMILES: CC1(C)c2ccccc2-c2ccc(-c3ccc(-c4nc(-c5ccccc5)nc(-c5ccc(-c6ccc7c(c6)C(C)(C)c6ccccc6-7)cc5)n4)cc3)cc21 ; c1ccc(-c2cccc(-c3cc(-c4cc(-n5c6ccccc6c6ccccc65)cc(-n5c6ccccc6c6ccccc65)c4)nc(-c4ccccc4)n3)c2)cc1 ; c1ccc(-c2cccc(-c3nc(-c4ccccc4)cc(-c4cc(-n5c6ccccc6c6ccccc65)cc(-n5c6ccccc6c6ccccc65)c4)n3)c2)cc1 ; c1ccc(-c2ccc(-c3cc(-c4cc(-n5c6ccccc6c6ccccc65)cc(-n5c6ccccc6c6ccccc65)c4)nc(-c4ccccc4)n3)cc2)cc1]

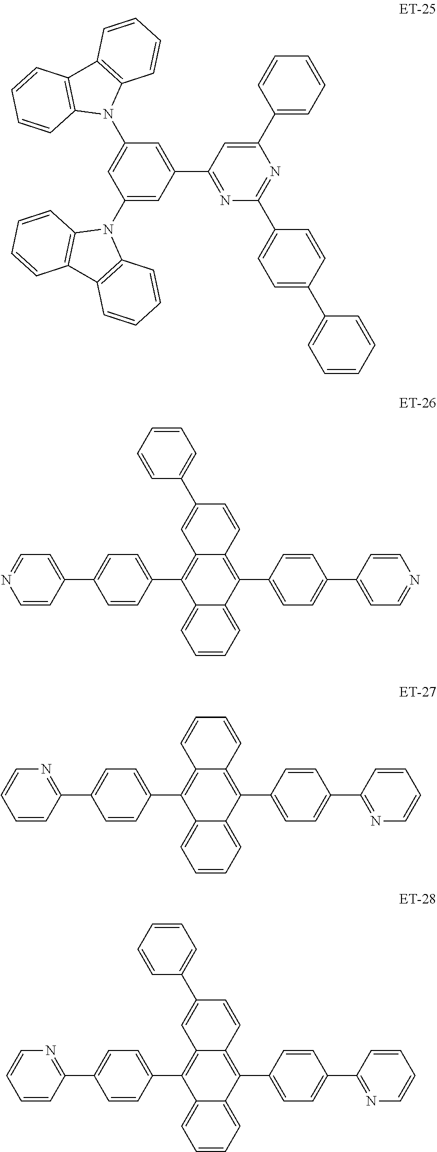 [SMILES: c1ccc(-c2ccc(-c3nc(-c4ccccc4)cc(-c4cc(-n5c6ccccc6c6ccccc65)cc(-n5c6ccccc6c6ccccc65)c4)n3)cc2)cc1 ; c1ccc(-c2ccc3c(-c4ccc(-c5ccncc5)cc4)c4ccccc4c(-c4ccc(-c5ccncc5)cc4)c3c2)cc1 ; c1ccc(-c2ccc(-c3c4ccccc4c(-c4ccc(-c5ccccn5)cc4)c4ccccc34)cc2)nc1 ; c1ccc(-c2ccc3c(-c4ccc(-c5ccccn5)cc4)c4ccccc4c(-c4ccc(-c5ccccn5)cc4)c3c2)cc1]

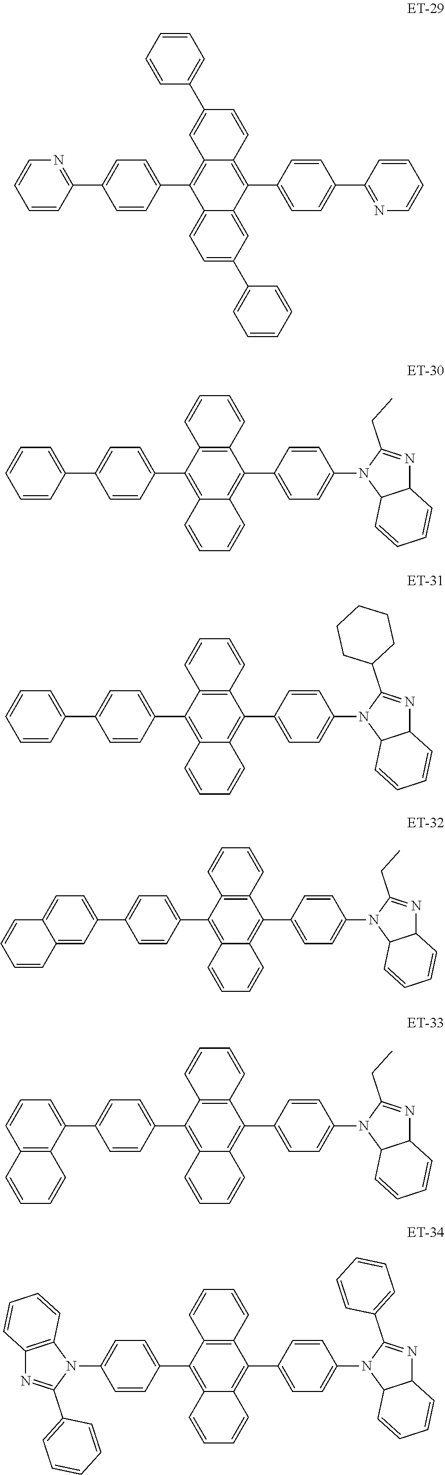

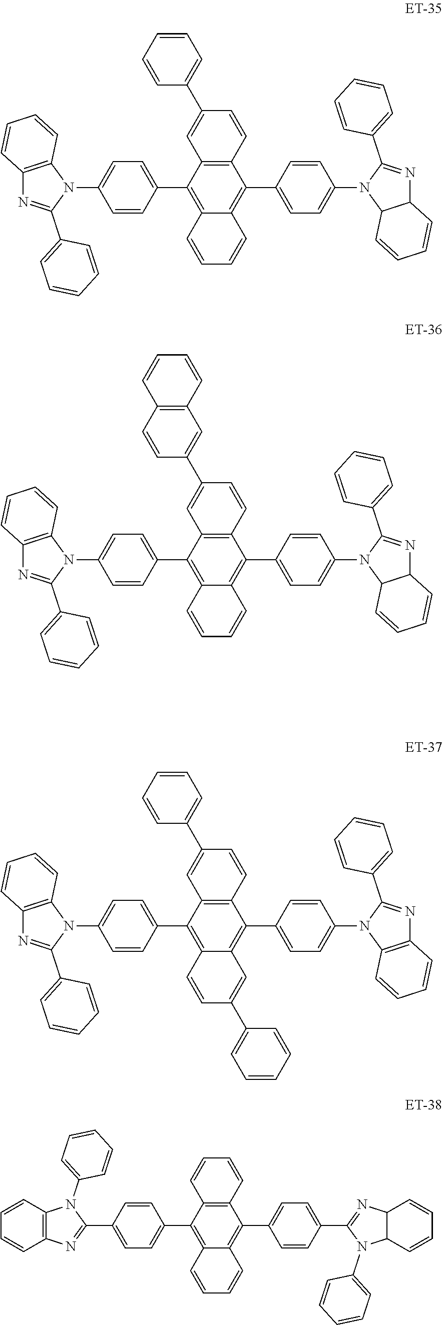 [SMILES: C1=CC2N=C(c3ccccc3)N(c3ccc(-c4c5ccccc5c(-c5ccc(-n6c(-c7ccccc7)nc7ccccc76)cc5)c5cc(-c6ccccc6)ccc45)cc3)C2C=C1 ; C1=CC2N=C(c3ccccc3)N(c3ccc(-c4c5ccccc5c(-c5ccc(-n6c(-c7ccccc7)nc7ccccc76)cc5)c5cc(-c6ccc7ccccc7c6)ccc45)cc3)C2C=C1 ; c1ccc(-c2ccc3c(-c4ccc(-n5c(-c6ccccc6)nc6ccccc65)cc4)c4cc(-c5ccccc5)ccc4c(-c4ccc(-n5c(-c6ccccc6)nc6ccccc65)cc4)c3c2)cc1 ; C1=CC2N=C(c3ccc(-c4c5ccccc5c(-c5ccc(-c6nc7ccccc7n6-c6ccccc6)cc5)c5ccccc45)cc3)N(c3ccccc3)C2C=C1]

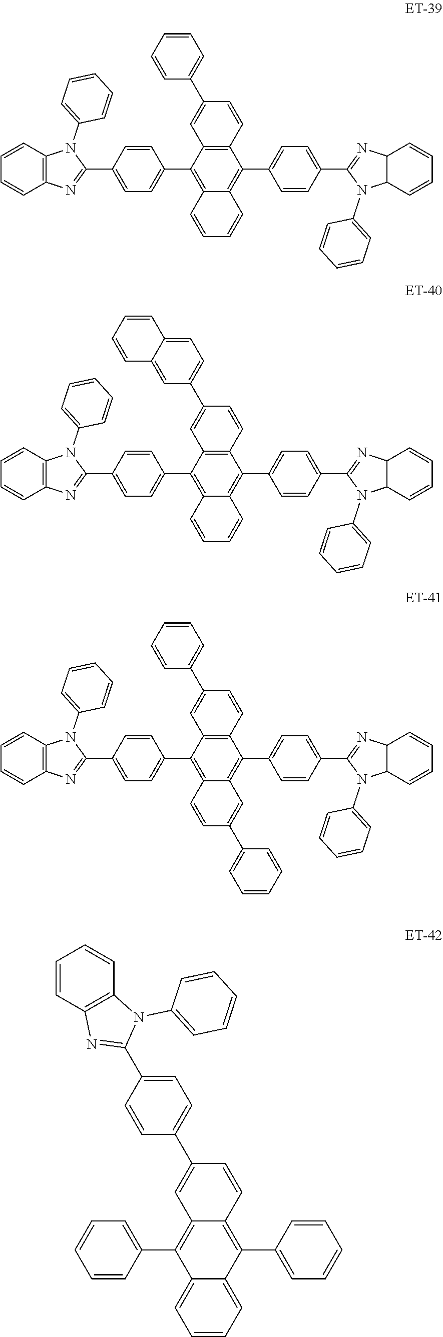 [SMILES: C1=CC2N=C(c3ccc(-c4c5ccccc5c(-c5ccc(-c6nc7ccccc7n6-c6ccccc6)cc5)c5cc(-c6ccccc6)ccc45)cc3)N(c3ccccc3)C2C=C1 ; C1=CC2N=C(c3ccc(-c4c5ccccc5c(-c5ccc(-c6nc7ccccc7n6-c6ccccc6)cc5)c5cc(-c6ccc7ccccc7c6)ccc45)cc3)N(c3ccccc3)C2C=C1 ; C1=CC2N=C(c3ccc(-c4c5ccc(-c6ccccc6)cc5c(-c5ccc(-c6nc7ccccc7n6-c6ccccc6)cc5)c5ccc(-c6ccccc6)cc45)cc3)N(c3ccccc3)C2C=C1 ; c1ccc(-c2c3ccccc3c(-c3ccccc3)c3cc(-c4ccc(-c5nc6ccccc6n5-c5ccccc5)cc4)ccc23)cc1]

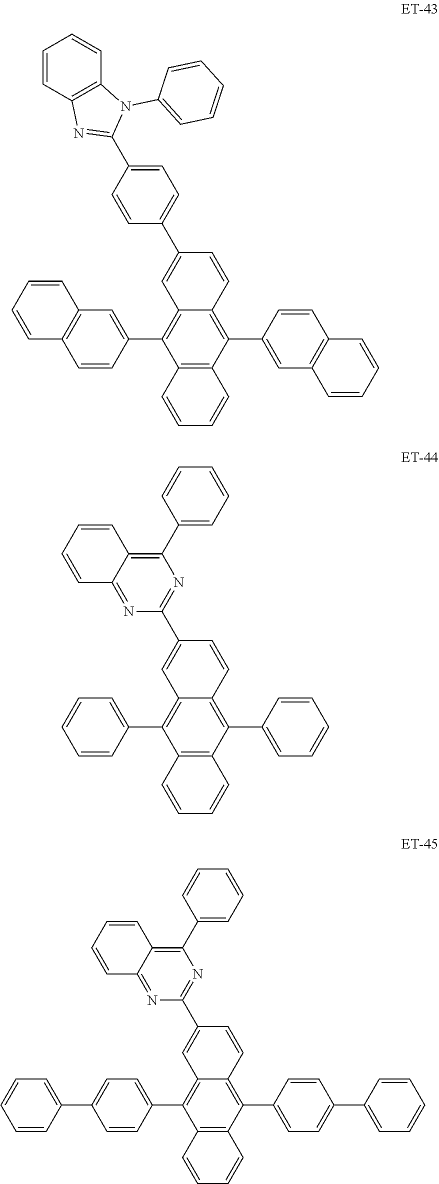

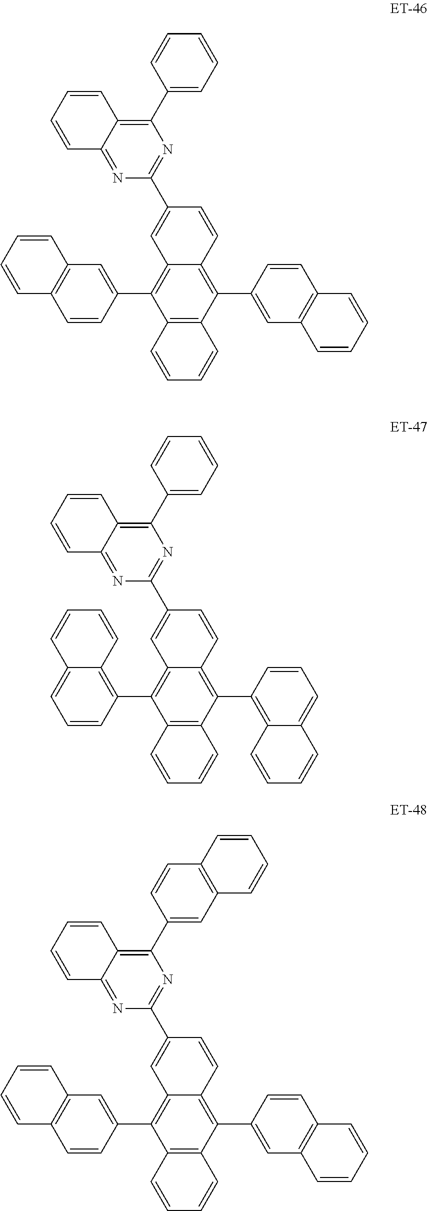 [SMILES: c1ccc(-c2nc(-c3ccc4c(-c5ccc6ccccc6c5)c5ccccc5c(-c5ccc6ccccc6c5)c4c3)nc3ccccc23)cc1 ; c1ccc(-c2nc(-c3ccc4c(-c5cccc6ccccc56)c5ccccc5c(-c5cccc6ccccc56)c4c3)nc3ccccc23)cc1 ; c1ccc2cc(-c3nc(-c4ccc5c(-c6ccc7ccccc7c6)c6ccccc6c(-c6ccc7ccccc7c6)c5c4)nc4ccccc34)ccc2c1]

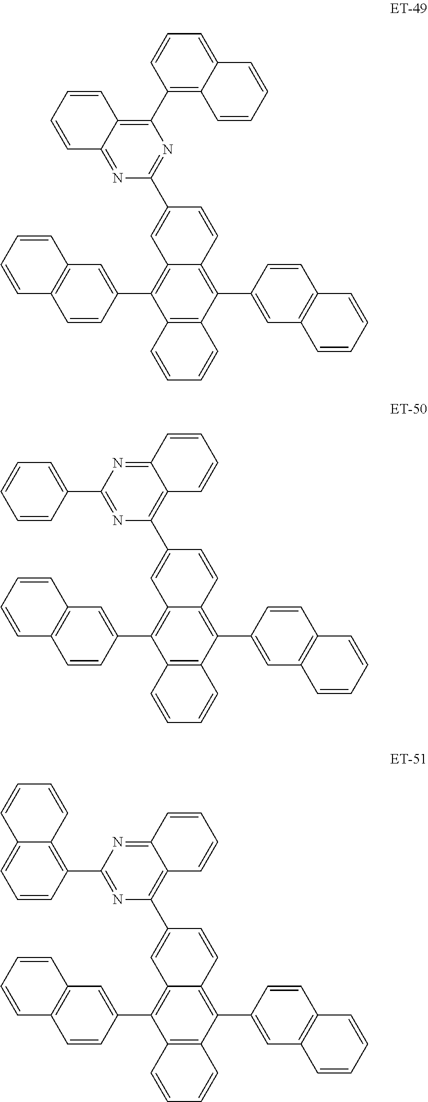 [SMILES: c1ccc2cc(-c3c4ccccc4c(-c4ccc5ccccc5c4)c4cc(-c5nc(-c6cccc7ccccc67)c6ccccc6n5)ccc34)ccc2c1 ; c1ccc(-c2nc(-c3ccc4c(-c5ccc6ccccc6c5)c5ccccc5c(-c5ccc6ccccc6c5)c4c3)c3ccccc3n2)cc1 ; c1ccc2cc(-c3c4ccccc4c(-c4ccc5ccccc5c4)c4cc(-c5nc(-c6cccc7ccccc67)nc6ccccc56)ccc34)ccc2c1]

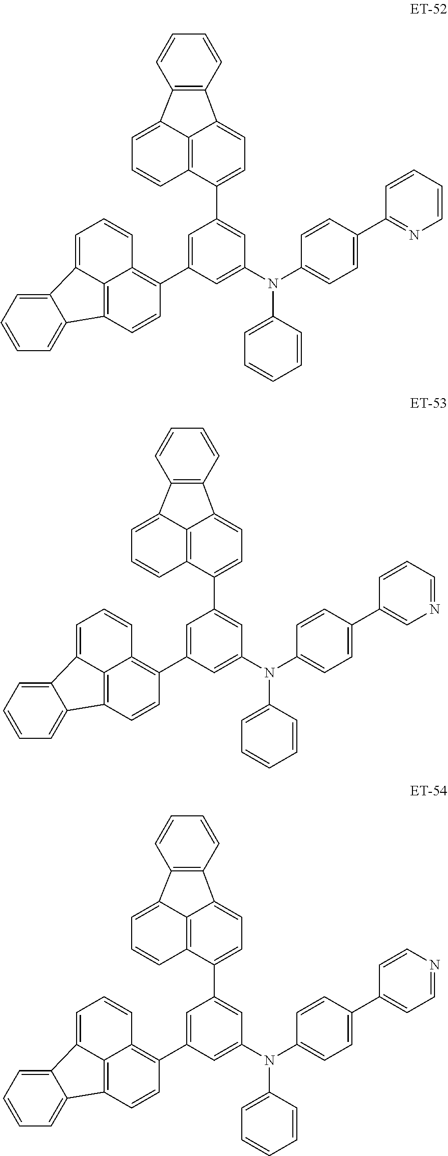 [SMILES: c1ccc(N(c2ccc(-c3ccccn3)cc2)c2cc(-c3ccc4c5c(cccc35)-c3ccccc3-4)cc(-c3ccc4c5c(cccc35)-c3ccccc3-4)c2)cc1 ; c1ccc(N(c2ccc(-c3cccnc3)cc2)c2cc(-c3ccc4c5c(cccc35)-c3ccccc3-4)cc(-c3ccc4c5c(cccc35)-c3ccccc3-4)c2)cc1 ; c1ccc(N(c2ccc(-c3ccncc3)cc2)c2cc(-c3ccc4c5c(cccc35)-c3ccccc3-4)cc(-c3ccc4c5c(cccc35)-c3ccccc3-4)c2)cc1]

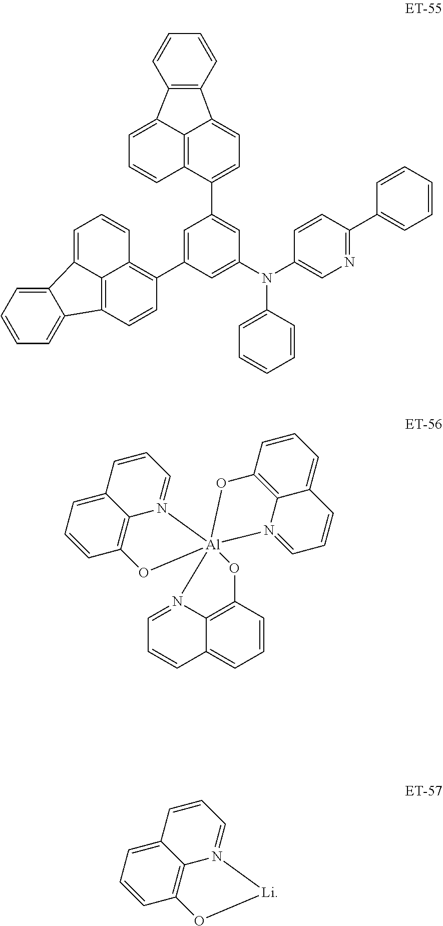

The device may further include an electron injection layer located between the electron transport layer and the cathode, and the electron injection layer material includes, but not limited to one or more combinations listed below: LiQ, LiF, NaCl, CsF, Li₂O, Cs₂CO₃, BaO, Na, Li or Ca.

### Preparation Method of the Compound of the Present Invention

The synthetic method of the compound of the present invention will be described briefly with detailed synthetic embodiments below.

The solvents and reagents used in the following synthetic examples, for example, aryl brominated compounds, 2-bromo-9,9′-dimethyl fluorene, 2-bromo-dibenzofuran, 2-bromo-dibenzothiophene, 4-bromo-biphenyl, 4-cyclohexyl bromobenzene, 4-(4′-cyclohexyl phenyl) bromobenzene, tri(dibenzylidene acetone) dipalladium, 1,3-bis(2,6-diisopropylphenyl) imidazolium chloride, toluene, tetrahydrofuran, petroleum ether, n-hexane, dichloromethane, acetone, sodium sulfate, ethyl acetate, ethanol, acetic acid, potassium phosphate, tri-tert-butylphosphine, potassium/sodium tert-butoxide, phenylamine, 1-naphthylamine, 2-naphthylamine, 2-aminobiphenyl, 2-amino-4-methoxy-5′-methoxy-1,2′-dinaphthalene, 2-amino-1,2′-dinaphthalene, 2-amino-4-methoxy-5′-methoxy-1,1′-dinaphthalene, 2-amino-1,1′-dinaphthalene, [1,1′-bis (diphenylphosphine)ferrocene] palladium dichloride, triphenylphosphine, and other chemical reagents may be purchased or customized from domestic chemical product markets, for example, purchased from Sinopharm Chemical Reagent Co., Ltd, Shanghai Titan Scientific Co., Ltd., XILONG Chemical Industry Co, Ltd, Sigma-Aldrich and J&K Reagent Company. Moreover, intermediates are customized by reagent companies, and a person skilled in the art also may synthesize intermediates by a commonly known method.

Representative synthesis path of the compound of Formula (I) of the present invention is as follows, but the synthetic method of the compound of the present invention is not limited thereto.

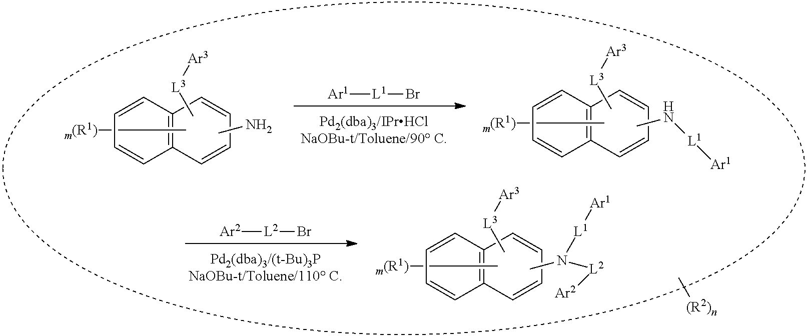

where, m, n, R¹, R², L¹, L², L³, Ar¹, Ar² and Ar³ and symbols in the Formula (I) have the same meaning.

More specifically, the following synthesis examples of the present invention exemplarily provide a detailed synthetic method of the representative compounds. It is confirmed that the mass spectrometer used in the following compounds is a ZAB-HS mass spectrometer for determination (manufactured by Britain Micromass).

### Synthesis of the Compounds of Preferred Embodiment 1

### Synthesis Example 1-1: Synthesis of the Compound P1

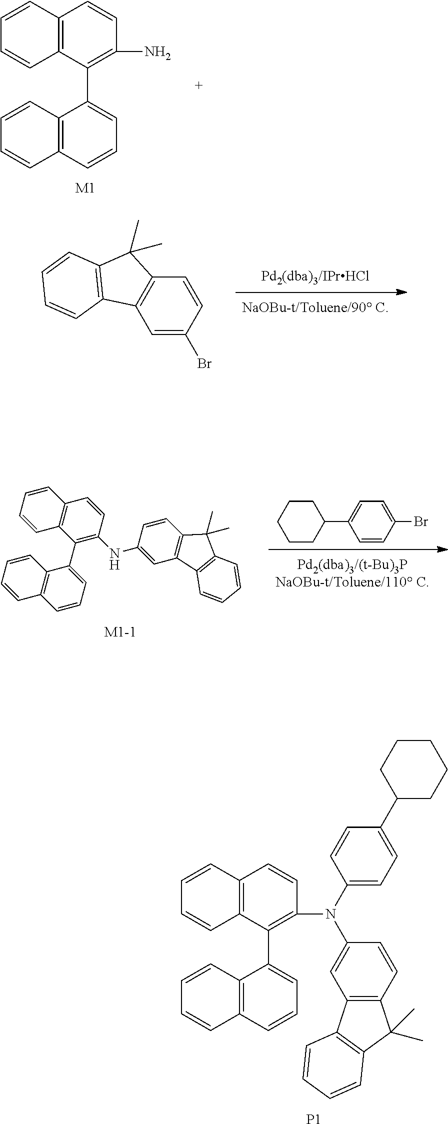

13.5 g (50 mmol) M1, 13.6 g (50 mmol) 3-bromo-9,9-dimethylfluorene, 0.9 g (1 mmol) tri(dibenzylidene acetone) dipalladium (namely, Pd₂(dba)₃), 0.5 g IPr.HCl, 500 mL toluene, and 14.4 g (150 mmol) sodium tert-butoxide (NaOBu-t) were added to a 1000 mL single-necked flask, and vacuumized for nitrogen exchange for 3 times, then the reaction was heated up to 90° C. for reacting for 5 h. The reaction was terminated at the end of the reaction. The flask was cooled to room temperature, and reaction liquid was separated, and organic phases were concentrated, then methanol was added and stirred for 1 h, and suction filtration was performed to obtain a faint yellow powder M1-1.

23 g (50 mmol) M1-1, 12 g (100 mmol) 4-cyclohexyl bromobenzene, 0.9 g (1 mmol) tri(dibenzylidene acetone) dipalladium (namely, Pd₂(dba)₃), 0.5 mL tri-tert-butylphosphine ((t-Bu)₃P), 500 mL toluene, and 14.4 g (150 mmol) sodium tert-butoxide (NaOBu-t) were added to a 1000 mL single-necked flask, and vacuumized for nitrogen exchange for 3 times, then the reaction was heated up to 110° C. for reacting for 5 h. The reaction was terminated at the end of the reaction. The flask was cooled to room temperature, and reaction liquid was separated, and organic phases were concentrated, then methanol was added and stirred for 1 h, and suction filtration was performed to obtain a faint yellow powder P1.

M/Z theoretical value: 619; ZAB-HS mass spectrometer (manufactured by Britain Micromass); M/Z measured value: 620.

### Synthesis Example 1-2: Synthesis of the Compound P3

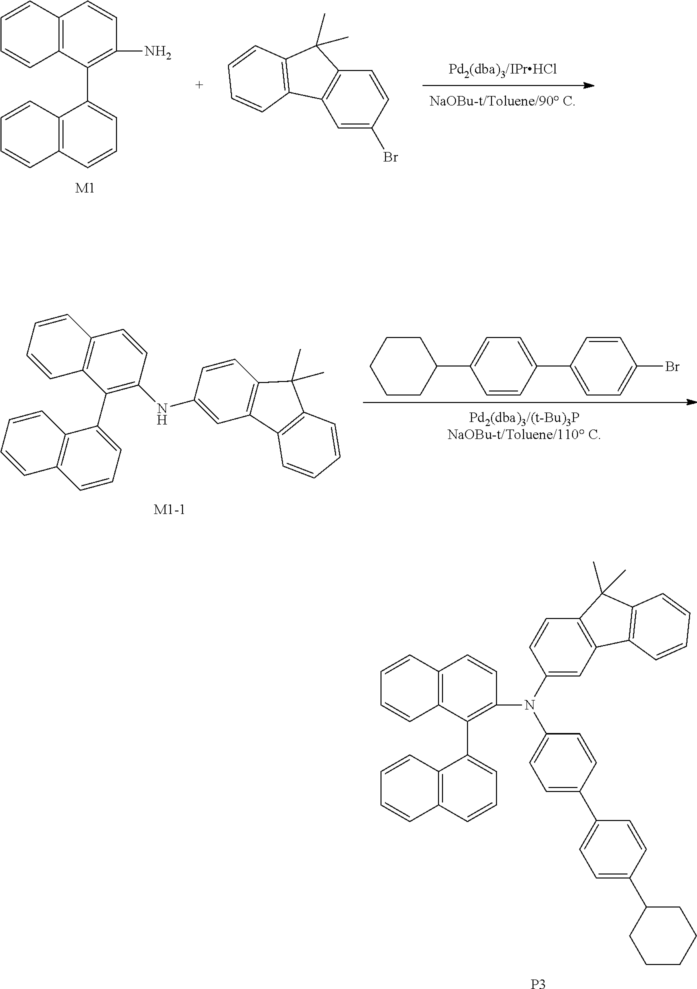

13.5 g (50 mmol) M1, 13.6 g (50 mmol) 3-bromo-9,9-dimethylfluorene, 0.9 g (1 mmol) tri(dibenzylidene acetone) dipalladium (namely, Pd₂(dba)₃), 0.5 g IPr.HCl, 500 mL toluene, and 14.4 g (150 mmol) sodium tert-butoxide (NaOBu-t) were added to a 1000 mL single-necked flask, and vacuumized for nitrogen exchange for 3 times, then the reaction was heated up to 90° C. for reacting for 5 h. The reaction was terminated at the end of the reaction. The flask was cooled to room temperature, and reaction liquid was separated, and organic phases were concentrated, then methanol was added and stirred for 1 h, and suction filtration was performed to obtain a faint yellow powder M1-1.

23 g (50 mmol) M1-1, 16 g (100 mmol) 4-(4-cyclohexyl phenyl) bromobenzene, 0.9 g (1 mmol) tri(dibenzylidene acetone) dipalladium (namely, Pd₂(dba)₃), 0.5 g tri-tert-butylphosphine ((t-Bu)₃P), 500 mL toluene, and 14.4 g (150 mmol) sodium tert-butoxide (NaOBu-t) were added to a 1000 mL single-necked flask, and vacuumized for nitrogen exchange for 3 times, then the reaction was heated up to 110° C. for reacting for 5 h. The reaction was terminated at the end of the reaction. The flask was cooled to room temperature, and reaction liquid was separated, and organic phases were concentrated, then methanol was added and stirred for 1 h, and suction filtration was performed to obtain a faint yellow powder P3.

M/Z theoretical value: 695; ZAB-HS mass spectrometer (manufactured by Britain Micromass); M/Z measured value: 696.

### Synthesis Example 1-3: Synthesis of the Compound P11

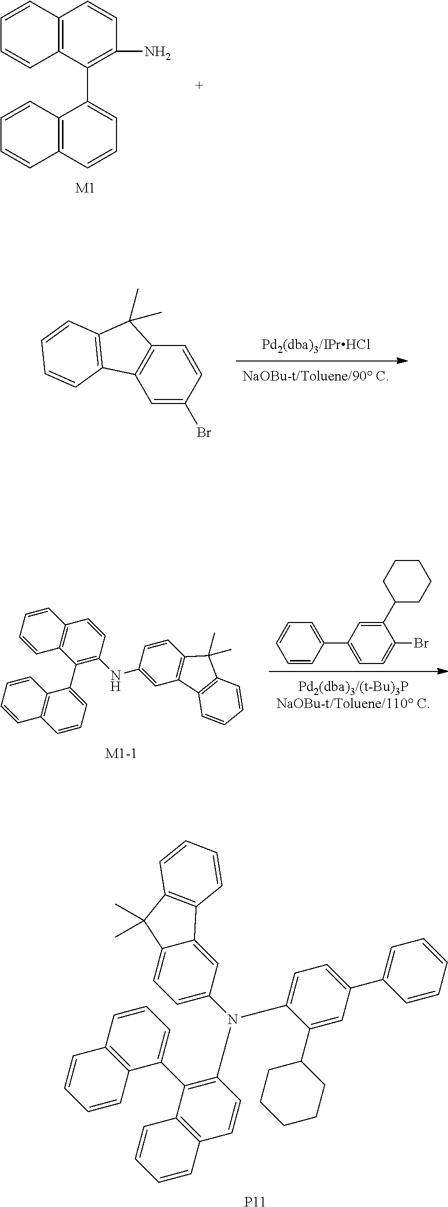

13.5 g (50 mmol) M1, 13.6 g (50 mmol) 3-bromo-9,9-dimethylfluorene, 0.9 g (1 mmol) tri(dibenzylidene acetone) dipalladium (namely, Pd₂(dba)₃), 0.5 g IPr.HCl, 500 mL toluene, and 14.4 g (150 mmol) sodium tert-butoxide (NaOBu-t) were added to a 1000 mL single-necked flask, and vacuumized for nitrogen exchange for 3 times, then the reaction was heated up to 90° C. for reacting for 5 h. The reaction was terminated at the end of the reaction. The flask was cooled to room temperature, and reaction liquid was separated, and organic phases were concentrated, then methanol was added and stirred for 1 h, and suction filtration was performed to obtain a faint yellow powder M1-1.

23 g (50 mmol) M1-1, 16 g (100 mmol) 2-cyclohexyl-4 phenylbromobenzene, 0.9 g (1 mmol) tri(dibenzylidene acetone) dipalladium (namely, Pd₂(dba)₃), 0.5 g tri-tert-butylphosphine ((t-Bu)₃P), 500 mL toluene, and 14.4 g (150 mmol) sodium tert-butoxide (NaOBu-t) were added to a 1000 mL single-necked flask, and vacuumized for nitrogen exchange for 3 times, then the reaction was heated up to 110° C. for reacting for 5 h. The reaction was terminated at the end of the reaction. The flask was cooled to room temperature, and reaction liquid was separated, and organic phases were concentrated, then methanol was added and stirred for 1 h, and suction filtration was performed to obtain a faint yellow powder P11.

M/Z theoretical value: 695; ZAB-HS mass spectrometer (manufactured by Britain Micromass); M/Z measured value: 696.

### Synthesis Example 1-4: Synthesis of the Compound P31

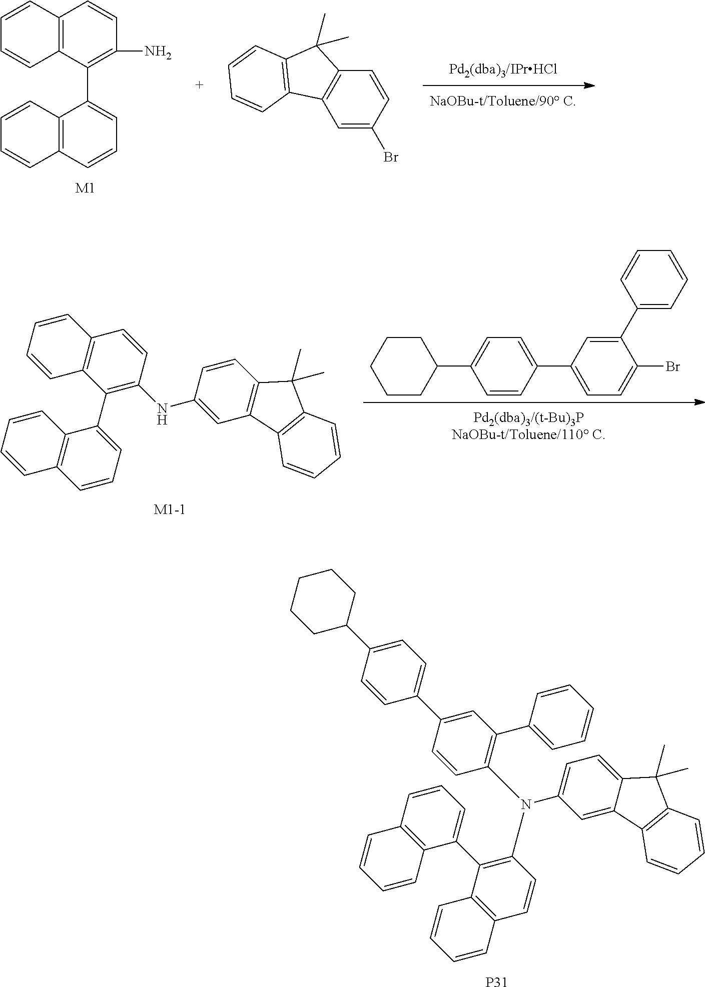

13.5 g (50 mmol) M1, 13.6 g (50 mmol) 3-bromo-9,9-dimethylfluorene, 0.9 g (1 mmol) tri(dibenzylidene acetone) dipalladium (namely, Pd₂(dba)₃), 0.5 g IPr.HCl, 500 mL toluene, and 14.4 g (150 mmol) sodium tert-butoxide (NaOBu-t) were added to a 1000 mL single-necked flask, and vacuumized for nitrogen exchange for 3 times, then the reaction was heated up to 90° C. for reacting for 5 h. The reaction was terminated at the end of the reaction. The flask was cooled to room temperature, and reaction liquid was separated, and organic phases were concentrated, then methanol was added and stirred for 1 h, and suction filtration was performed to obtain a faint yellow powder M1-1.

23 g (50 mmol) M1-1, 20 g (100 mmol) 2-phenyl-4(4′4-cyclohexyl phenyl) bromobenzene, 0.9 g (1 mmol) tri(dibenzylidene acetone) dipalladium (namely, Pd₂(dba)₃), 0.5 g tri-tert-butylphosphine ((t-Bu)₃P), 500 mL toluene, and 14.4 g (150 mmol) sodium tert-butoxide (NaOBu-t) were added to a 1000 mL single-necked flask, and vacuumized for nitrogen exchange for 3 times, then the reaction was heated up to 110° C. for reacting for 5 h. The reaction was terminated at the end of the reaction. The flask was cooled to room temperature, and reaction liquid was separated, and organic phases were concentrated, then methanol was added and stirred for 1 h, and suction filtration was performed to obtain a faint yellow powder P31.

M/Z theoretical value: 771; ZAB-HS mass spectrometer (manufactured by Britain Micromass); M/Z measured value: 772.

### Synthesis Example P1-5: Synthesis of the Compound P37

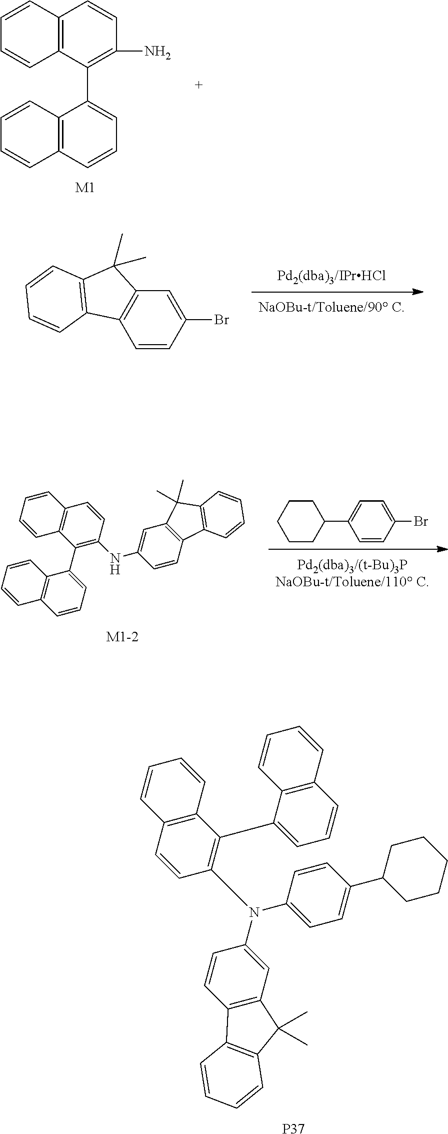

13.5 g (50 mmol) M1, 13.6 g (50 mmol) 3-bromo-9,9-dimethylfluorene, 0.9 g (1 mmol) tri(dibenzylidene acetone) dipalladium (namely, Pd₂(dba)₃), 0.5 g IPr.HCl, 500 mL toluene, and 14.4 g (150 mmol) sodium tert-butoxide (NaOBu-t) were added to a 1000 mL single-necked flask, and vacuumized for nitrogen exchange for 3 times, then the reaction was heated up to 90° C. for reacting for 5 h. The reaction was terminated at the end of the reaction. The flask was cooled to room temperature, and reaction liquid was separated, and organic phases were concentrated, then methanol was added and stirred for 1 h, and suction filtration was performed to obtain a faint yellow powder M1-2.

23 g (50 mmol) M1-2, 12 g (100 mmol) 4-cyclohexyl bromobenzene, 0.9 g (1 mmol) tri(dibenzylidene acetone) dipalladium (namely, Pd₂(dba)₃), 0.5 g tri-tert-butylphosphine ((t-Bu)₃P), 500 mL toluene, and 14.4 g (150 mmol) sodium tert-butoxide (NaOBu-t) were added to a 1000 mL single-necked flask, and vacuumized for nitrogen exchange for 3 times, then the reaction was heated up to 110° C. for reacting for 5 h. The reaction was terminated at the end of the reaction. The flask was cooled to room temperature, and reaction liquid was separated, and organic phases were concentrated, then methanol was added and stirred for 1 h, and suction filtration was performed to obtain a faint yellow powder P37.

M/Z theoretical value: 619; ZAB-HS mass spectrometer (manufactured by Britain Micromass); M/Z measured value: 620.

### Synthesis Example 1-6: Synthesis of the Compound P39

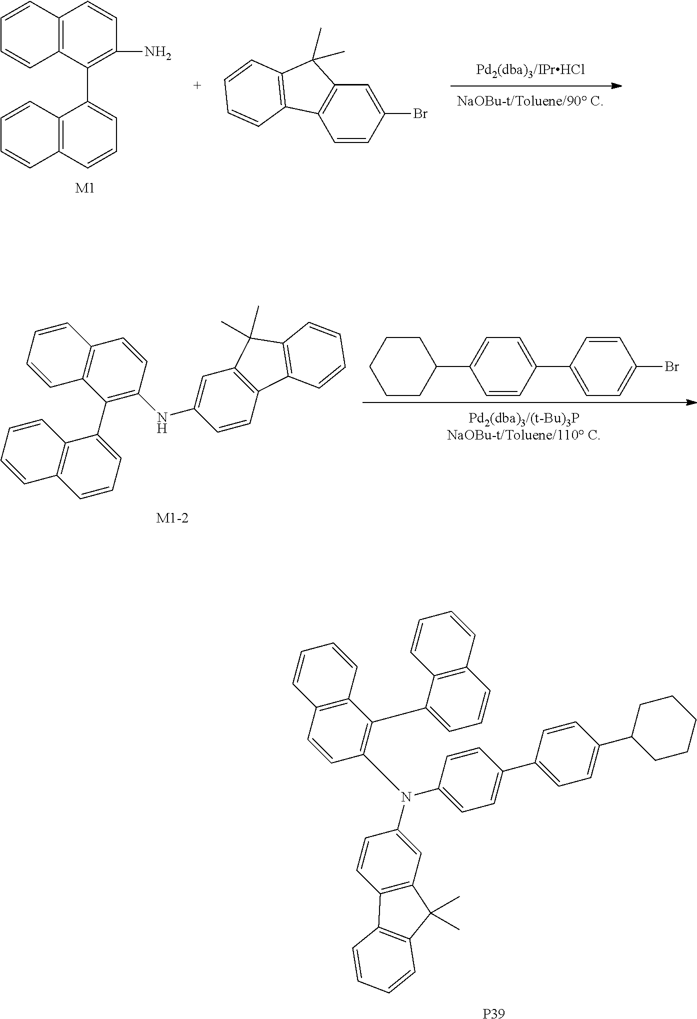

13.5 g (50 mmol) M1, 13.6 g (50 mmol) 3-bromo-9,9-dimethylfluorene, 0.9 g (1 mmol) tri(dibenzylidene acetone) dipalladium (namely, Pd₂(dba)₃), 0.5 g IPr.HCl, 500 mL toluene, and 14.4 g (150 mmol) sodium tert-butoxide (NaOBu-t) were added to a 1000 mL single-necked flask, and vacuumized for nitrogen exchange for 3 times, then the reaction was heated up to 90° C. for reacting for 5 h. The reaction was terminated at the end of the reaction. The flask was cooled to room temperature, and reaction liquid was separated, and organic phases were concentrated, then methanol was added and stirred for 1 h, and suction filtration was performed to obtain a faint yellow powder M1-2.

23 g (50 mmol) M1-2, 16 g (100 mmol) 4-cyclohexyl bromobenzene, 0.9 g (1 mmol) tri(dibenzylidene acetone) dipalladium (namely, Pd₂(dba)₃), 0.5 g tri-tert-butylphosphine ((t-Bu)₃P), 500 mL toluene, and 14.4 g (150 mmol) sodium tert-butoxide (NaOBu-t) were added to a 1000 mL single-necked flask, and vacuumized for nitrogen exchange for 3 times, then the reaction was heated up to 110° C. for reacting for 5 h. The reaction was terminated at the end of the reaction. The flask was cooled to room temperature, and reaction liquid was separated, and organic phases were concentrated, then methanol was added and stirred for 1 h, and suction filtration was performed to obtain a faint yellow powder P39.

M/Z theoretical value: 695; ZAB-HS mass spectrometer (manufactured by Britain Micromass); M/Z measured value: 696.

### Synthesis Example 1-7: Synthesis of the Compound P61

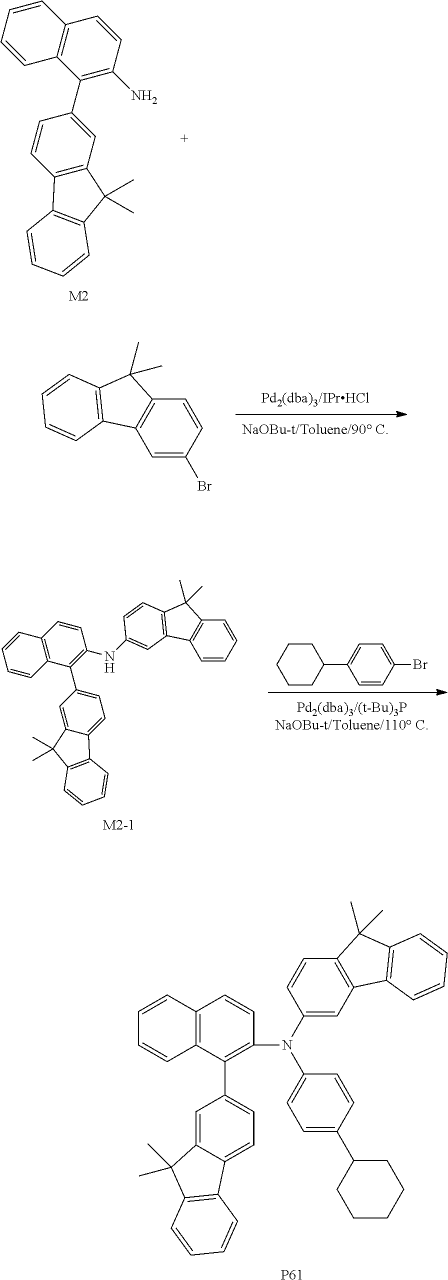

16.5 g (50 mmol) M2, 13.6 g (50 mmol) 3-bromo-9,9-dimethylfluorene, 0.9 g (1 mmol) tri(dibenzylidene acetone) dipalladium (namely, Pd₂(dba)₃), 0.5 g IPr.HCl, 500 mL toluene, and 14.4 g (150 mmol) sodium tert-butoxide (NaOBu-t) were added to a 1000 mL single-necked flask, and vacuumized for nitrogen exchange for 3 times, then the reaction was heated up to 90° C. for reacting for 5 h. The reaction was terminated at the end of the reaction. The flask was cooled to room temperature, and reaction liquid was separated, and organic phases were concentrated, then methanol was added and stirred for 1 h, and suction filtration was performed to obtain a faint yellow powder M2-1.

26.5 g (50 mmol) M2-1, 12 g (100 mmol) 4-cyclohexyl bromobenzene, 0.9 g (1 mmol) tri(dibenzylidene acetone) dipalladium (namely, Pd₂(dba)₃), 0.5 g tri-tert-butylphosphine ((t-Bu)₃P), 500 mL toluene, and 14.4 g (150 mmol) sodium tert-butoxide (NaOBu-t) were added to a 1000 mL single-necked flask, and vacuumized for nitrogen exchange for 3 times, then the reaction was heated up to 110° C. for reacting for 5 h. The reaction was terminated at the end of the reaction. The flask was cooled to room temperature, and reaction liquid was separated, and organic phases were concentrated, then methanol was added and stirred for 1 h, and suction filtration was performed to obtain a faint yellow powder P61.

M/Z theoretical value: 685; ZAB-HS mass spectrometer (manufactured by Britain Micromass); M/Z measured value: 686.

### Synthesis Example 1-8: Synthesis of the Compound P62

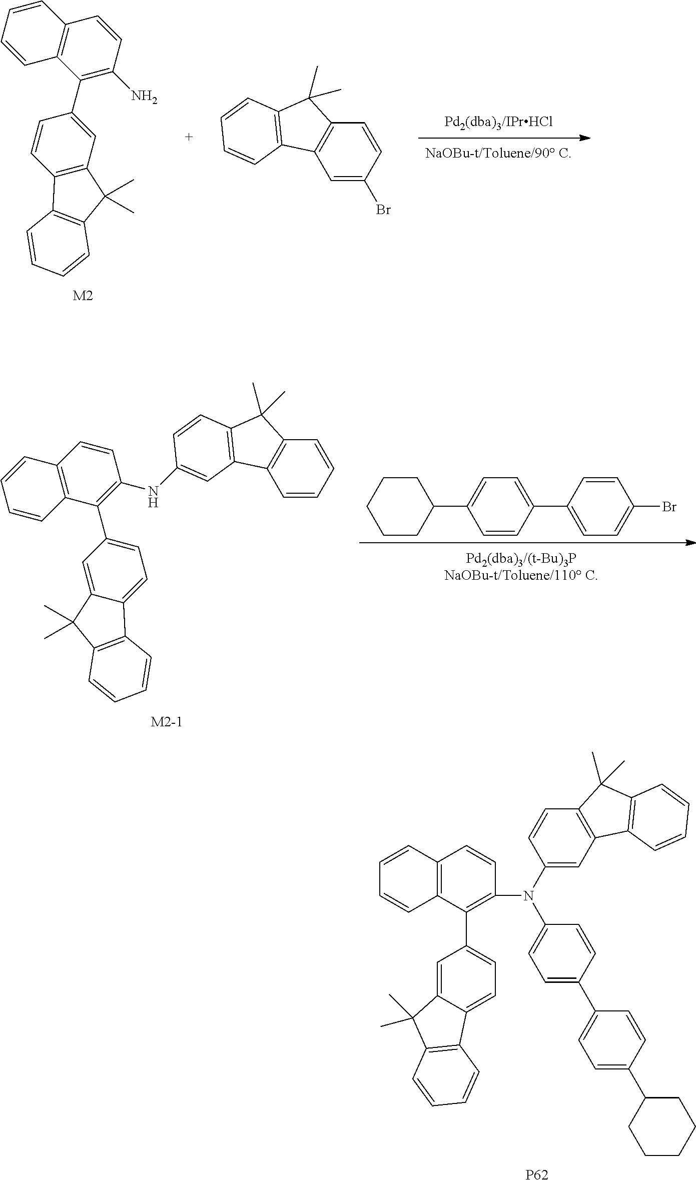

16.5 g (50 mmol) M2, 13.6 g (50 mmol) 3-bromo-9,9-dimethylfluorene, 0.9 g (1 mmol) tri(dibenzylidene acetone) dipalladium (namely, Pd₂(dba)₃), 0.5 g IPr.HCl, 500 mL toluene, and 14.4 g (150 mmol) sodium tert-butoxide (NaOBu-t) were added to a 1000 mL single-necked flask, and vacuumized for nitrogen exchange for 3 times, then the reaction was heated up to 90° C. for reacting for 5 h. The reaction was terminated at the end of the reaction. The flask was cooled to room temperature, and reaction liquid was separated, and organic phases were concentrated, then methanol was added and stirred for 1 h, and suction filtration was performed to obtain a faint yellow powder M2-1.

26.5 g (50 mmol) M2-1, 16 g (100 mmol) 4-(4,-cyclohexyl phenyl) bromobenzene, 0.9 g (1 mmol) tri(dibenzylidene acetone) dipalladium (namely, Pd₂(dba)₃), 0.5 g tri-tert-butylphosphine ((t-Bu)₃P), 500 mL toluene, and 14.4 g (150 mmol) sodium tert-butoxide (NaOBu-t) were added to a 1000 mL single-necked flask, and vacuumized for nitrogen exchange for 3 times, then the reaction was heated up to 110° C. for reacting for 5 h. The reaction was terminated at the end of the reaction. The flask was cooled to room temperature, and reaction liquid was separated, and organic phases were concentrated, then methanol was added and stirred for 1 h, and suction filtration was performed to obtain a faint yellow powder P62.

M/Z theoretical value: 762; ZAB-HS mass spectrometer (manufactured by Britain Micromass); M/Z measured value: 763.

### Synthesis Example 1-9: Synthesis of the Compound P73

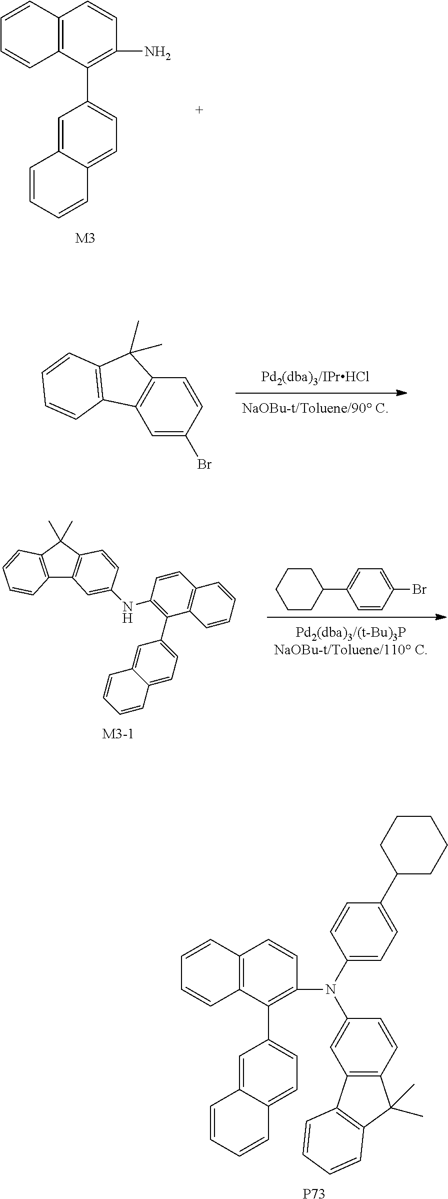

13.5 g (50 mmol) M3, 13.6 g (50 mmol) 3-bromo-9,9-dimethylfluorene, 0.9 g (1 mmol) tri(dibenzylidene acetone) dipalladium (namely, Pd₂(dba)₃), 0.5 g IPr.HCl, 500 mL toluene, and 14.4 g (150 mmol) sodium tert-butoxide (NaOBu-t) were added to a 1000 mL single-necked flask, and vacuumized for nitrogen exchange for 3 times, then the reaction was heated up to 90° C. for reacting for 5 h. The reaction was terminated at the end of the reaction. The flask was cooled to room temperature, and reaction liquid was separated, and organic phases were concentrated, then methanol was added and stirred for 1 h, and suction filtration was performed to obtain a faint yellow M3-1.

23 g (50 mmol) M3-1, 12 g (100 mmol) 4-cyclohexyl bromobenzene, 0.9 g (1 mmol) tri(dibenzylidene acetone) dipalladium (namely, Pd₂(dba)₃), 0.5 mL tri-tert-butylphosphine ((t-Bu)₃P), 500 mL toluene, and 14.4 g (150 mmol) sodium tert-butoxide (NaOBu-t) were added to a 1000 mL single-necked flask, and vacuumized for nitrogen exchange for 3 times, then the reaction was heated up to 110° C. for reacting for 5 h. The reaction was terminated at the end of the reaction. The flask was cooled to room temperature, and reaction liquid was separated, and organic phases were concentrated, then methanol was added and stirred for 1 h, and suction filtration was performed to obtain a faint yellow powder P73.

M/Z theoretical value: 619; ZAB-HS mass spectrometer (manufactured by Britain Micromass); M/Z measured value: 620.

### Synthesis Example 1-10: Synthesis of the Compound P75

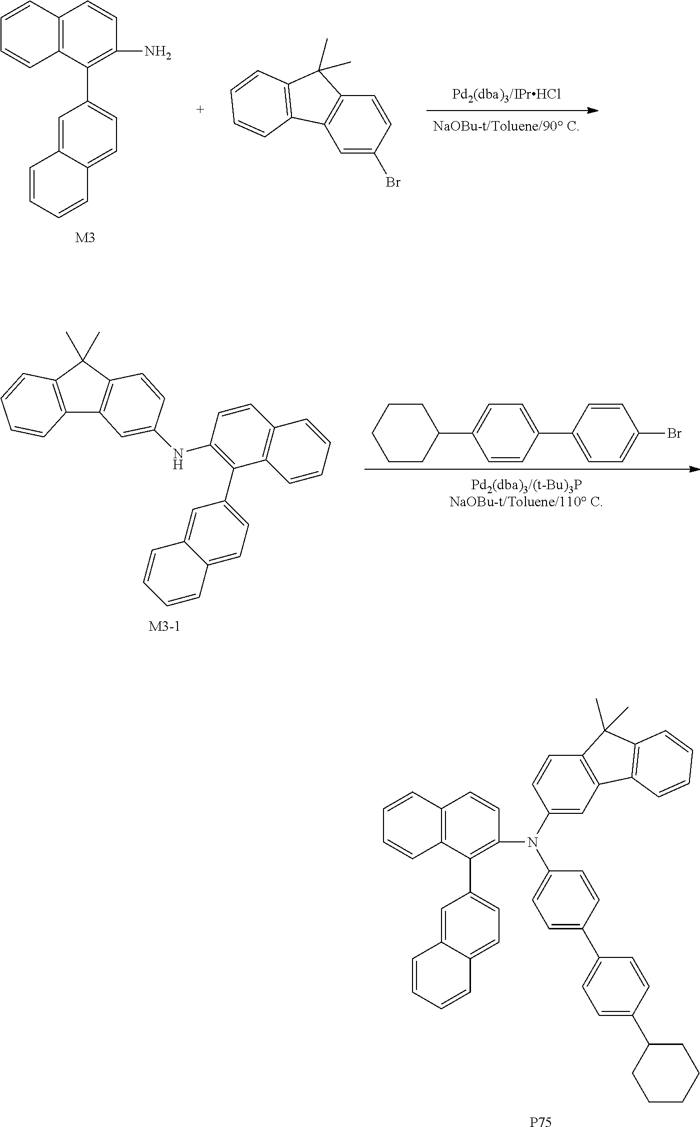

13.5 g (50 mmol) M3, 13.6 g (50 mmol) 3-bromo-9,9-dimethylfluorene, 0.9 g (1 mmol) tri(dibenzylidene acetone) dipalladium (namely, Pd₂(dba)₃), 0.5 g IPr.HCl, 500 mL toluene, and 14.4 g (150 mmol) sodium tert-butoxide (NaOBu-t) were added to a 1000 mL single-necked flask, and vacuumized for nitrogen exchange for 3 times, then the reaction was heated up to 90° C. for reacting for 5 h. The reaction was terminated at the end of the reaction. The flask was cooled to room temperature, and reaction liquid was separated, and organic phases were concentrated, then methanol was added and stirred for 1 h, and suction filtration was performed to obtain a faint yellow M3-1.

23 g (50 mmol) M3-1, 16 g (100 mmol) 4-(4,-cyclohexyl phenyl) bromobenzene, 0.9 g (1 mmol) tri(dibenzylidene acetone) dipalladium (namely, Pd₂(dba)₃), 0.5 g tri-tert-butylphosphine ((t-Bu)₃P), 500 mL toluene, and 14.4 g (150 mmol) sodium tert-butoxide (NaOBu-t) were added to a 1000 mL single-necked flask, and vacuumized for nitrogen exchange for 3 times, then the reaction was heated up to 110° C. for reacting for 5 h. The reaction was terminated at the end of the reaction. The flask was cooled to room temperature, and reaction liquid was separated, and organic phases were concentrated, then methanol was added and stirred for 1 h, and suction filtration was performed to obtain a faint yellow powder P75.

M/Z theoretical value: 695; ZAB-HS mass spectrometer (manufactured by Britain Micromass); M/Z measured value: 696.

### Synthesis Example 1-11: Synthesis of the Compound P97

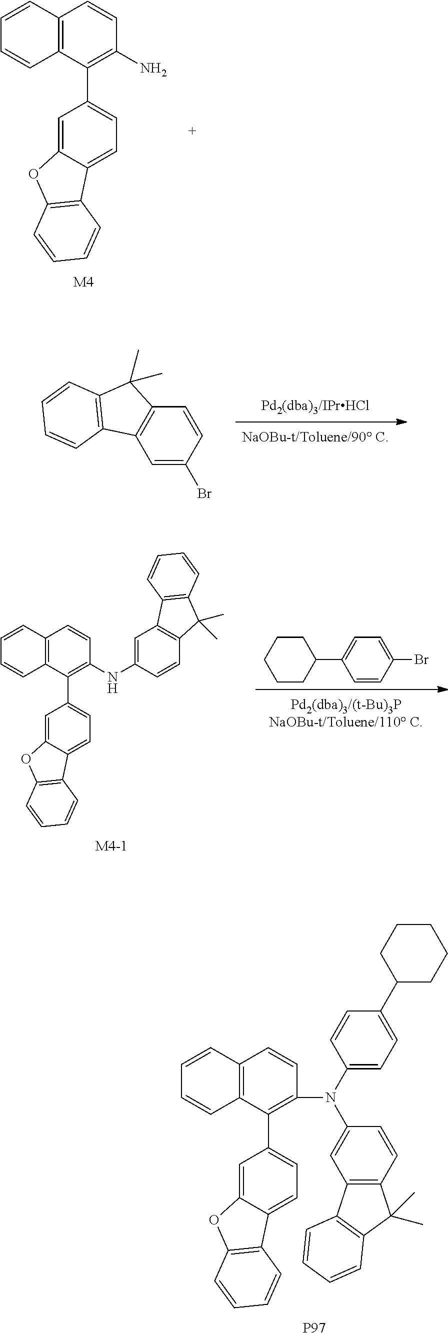

15.5 g (50 mmol) M4, 13.6 g (50 mmol) 3-bromo-9,9-dimethylfluorene, 0.9 g (1 mmol) tri(dibenzylidene acetone) dipalladium (namely, Pd₂(dba)₃), 0.5 g IPr.HCl, 500 mL toluene, and 14.4 g (150 mmol) sodium tert-butoxide (NaOBu-t) were added to a 1000 mL single-necked flask, and vacuumized for nitrogen exchange for 3 times, then the reaction was heated up to 90° C. for reacting for 5 h. The reaction was terminated at the end of the reaction. The flask was cooled to room temperature, and reaction liquid was separated, and organic phases were concentrated, then methanol was added and stirred for 1 h, and suction filtration was performed to obtain a faint yellow powder M4-1.

25 g (50 mmol) M4-1, 12 g (100 mmol) 4-cyclohexyl bromobenzene, 0.9 g (1 mmol) tri(dibenzylidene acetone) dipalladium (namely, Pd₂(dba)₃), 0.5 g tri-tert-butylphosphine ((t-Bu)₃P), 500 mL toluene, and 14.4 g (150 mmol) sodium tert-butoxide (NaOBu-t) were added to a 1000 mL single-necked flask, and vacuumized for nitrogen exchange for 3 times, then the reaction was heated up to 110° C. for reacting for 5 h. The reaction was terminated at the end of the reaction. The flask was cooled to room temperature, and reaction liquid was separated, and organic phases were concentrated, then methanol was added and stirred for 1 h, and suction filtration was performed to obtain a faint yellow powder P97.

M/Z theoretical value: 659; ZAB-HS mass spectrometer (manufactured by Britain Micromass); M/Z measured value: 660.

### Synthesis Example 1-12: Synthesis of the Compound P109

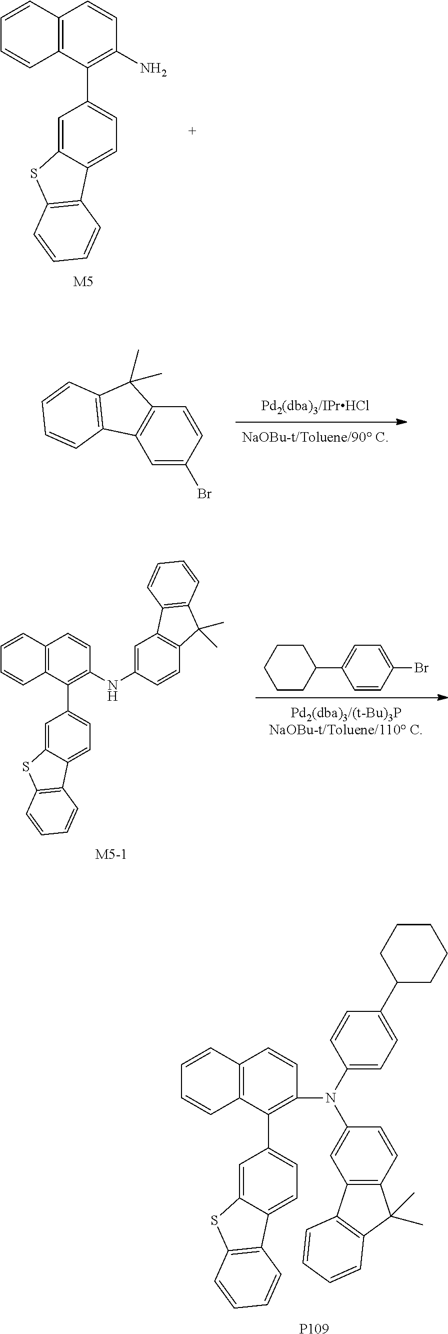

16.2 g (50 mmol) M5, 13.6 g (50 mmol) 3-bromo-9,9-dimethylfluorene, 0.9 g (1 mmol) tri(dibenzylidene acetone) dipalladium (namely, Pd₂(dba)₃), 0.5 g IPr.HCl, 500 mL toluene, and 14.4 g (150 mmol) sodium tert-butoxide (NaOBu-t) were added to a 1000 mL single-necked flask, and vacuumized for nitrogen exchange for 3 times, then the reaction was heated up to 90° C. for reacting for 5 h. The reaction was terminated at the end of the reaction. The flask was cooled to room temperature, and reaction liquid was separated, and organic phases were concentrated, then methanol was added and stirred for 1 h, and suction filtration was performed to obtain a faint yellow powder M5-1.

26 g (50 mmol) M5-1, 12 g (100 mmol) 4-cyclohexyl bromobenzene, 0.9 g (1 mmol) tri(dibenzylidene acetone) dipalladium (namely, Pd₂(dba)₃), 0.5 g tri-tert-butylphosphine ((t-Bu)₃P), 500 mL toluene, and 14.4 g (150 mmol) sodium tert-butoxide (NaOBu-t) were added to a 1000 mL single-necked flask, and vacuumized for nitrogen exchange for 3 times, then the reaction was heated up to 110° C. for reacting for 5 h. The reaction was terminated at the end of the reaction. The flask was cooled to room temperature, and reaction liquid was separated, and organic phases were concentrated, then methanol was added and stirred for 1 h, and suction filtration was performed to obtain a faint yellow powder P109.

M/Z theoretical value: 675; ZAB-HS mass spectrometer (manufactured by Britain Micromass); M/Z measured value: 676.

### Synthesis Example 1-13: Synthesis of the Compound P121

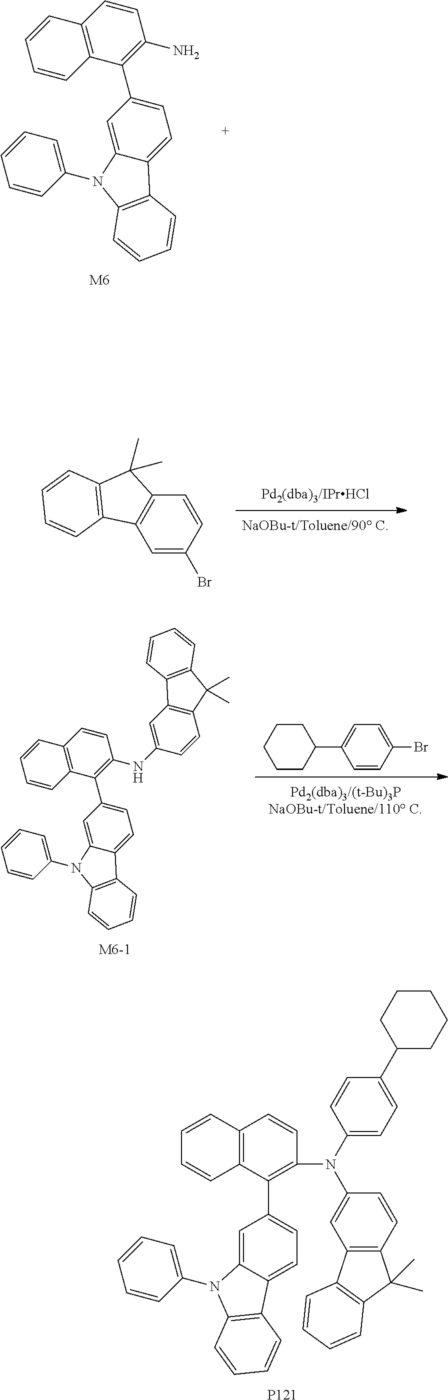

19.5 g (50 mmol) M6, 13.6 g (50 mmol) 3-bromo-9,9-dimethyfluorene, 0.9 g (1 mmol) tri(dibenzylidene acetone) dipalladium (namely, Pd₂(dba)₃), 0.5 g IPr.HCl, 500 mL toluene, and 14.4 g (150 mmol) sodium tert-butoxide (NaOBu-t) were added to a 1000 mL single-necked flask, and vacuumized for nitrogen exchange for 3 times, then the reaction was heated up to 90° C. for reacting for 5 h. The reaction was terminated at the end of the reaction. The flask was cooled to room temperature, and reaction liquid was separated, and organic phases were concentrated, then methanol was added and stirred for 1 h, and suction filtration was performed to obtain a faint yellow M6-1.

29 g (50 mmol) M6-1, 12 g (100 mmol) 4-cyclohexyl bromobenzene, 0.9 g (1 mmol) tri(dibenzylidene acetone) dipalladium (namely, Pd₂(dba)₃), 0.5 g tri-tert-butylphosphine ((t-Bu)₃P), 500 mL toluene, and 14.4 g (150 mmol) sodium tert-butoxide (NaOBu-t) were added to a 1000 mL single-necked flask, and vacuumized for nitrogen exchange for 3 times, then the reaction was heated up to 110° C. for reacting for 5 h. The reaction was terminated at the end of the reaction. The flask was cooled to room temperature, and reaction liquid was separated, and organic phases were concentrated, then methanol was added and stirred for 1 h, and suction filtration was performed to obtain a faint yellow powder P121.

M/Z theoretical value: 734; ZAB-HS mass spectrometer (manufactured by Britain Micromass); M/Z measured value: 735.

### Synthesis Example 1-14: Synthesis of the Compound P133

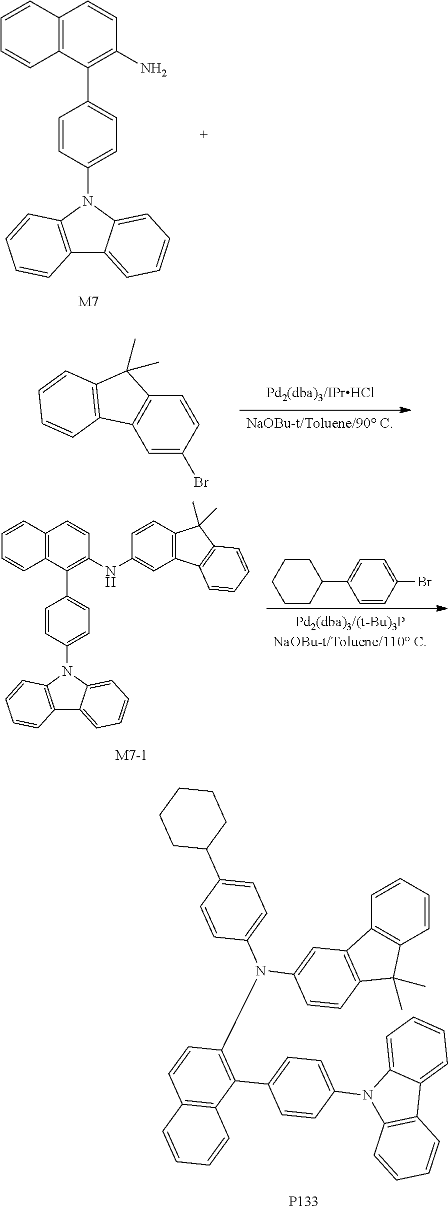

19.5 g (50 mmol) M7, 13.6 g (50 mmol) 3-bromo-9,9-dimethylfluorene, 0.9 g (1 mmol) tri(dibenzylidene acetone) dipalladium (namely, Pd₂(dba)₃), 0.5 g IPr.HCl, 500 mL toluene, and 14.4 g (150 mmol) sodium tert-butoxide (NaOBu-t) were added to a 1000 mL single-necked flask, and vacuumized for nitrogen exchange for 3 times, then the reaction was heated up to 90° C. for reacting for 5 h. The reaction was terminated at the end of the reaction. The flask was cooled to room temperature, and reaction liquid was separated, and organic phases were concentrated, then methanol was added and stirred for 1 h, and suction filtration was performed to obtain a faint yellow powder M7-1.

29 g (50 mmol) M7-1, 12 g (100 mmol) 4-cyclohexyl bromobenzene, 0.9 g (1 mmol) tri(dibenzylidene acetone) dipalladium (namely, Pd₂(dba)₃), 0.5 g tri-tert-butylphosphine ((t-Bu)₃P), 500 mL toluene, and 14.4 g (150 mmol) sodium tert-butoxide (NaOBu-t) were added to a 1000 mL single-necked flask, and vacuumized for nitrogen exchange for 3 times, then the reaction was heated up to 110° C. for reacting for 5 h. The reaction was terminated at the end of the reaction. The flask was cooled to room temperature, and reaction liquid was separated, and organic phases were concentrated, then methanol was added and stirred for 1 h, and suction filtration was performed to obtain a faint yellow powder P133.

M/Z theoretical value: 734; ZAB-HS mass spectrometer (manufactured by Britain Micromass); M/Z measured value: 735.

### Synthesis Example 1-15: Synthesis of the Compound P173

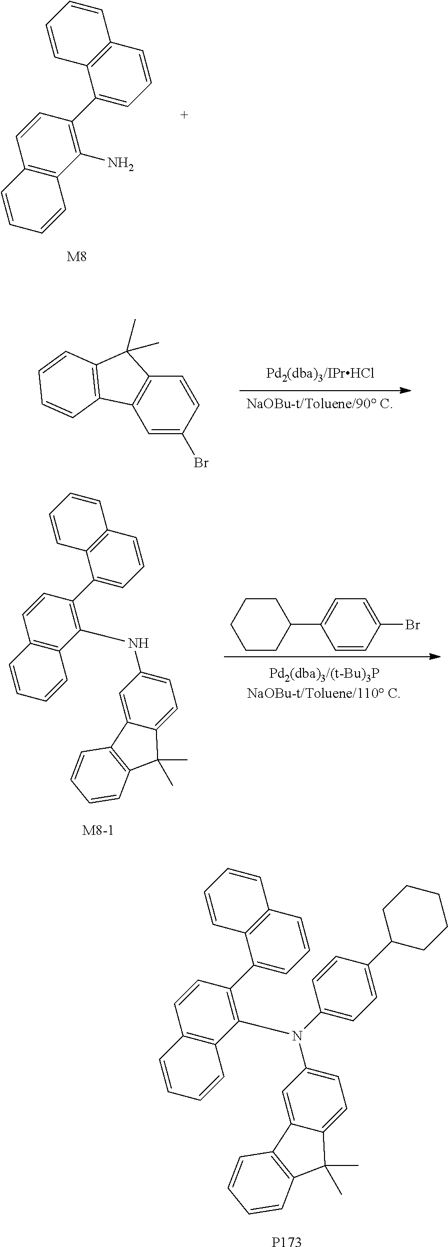

13.5 g (50 mmol) M8, 13.6 g (50 mmol) 3-bromo-9,9-dimethylfluorene, 0.9 g (1 mmol) tri(dibenzylidene acetone) dipalladium (namely. Pd₂(dba)₃), 0.5 g IPr.HCl, 500 mL toluene, and 14.4 g (150 mmol) sodium tert-butoxide (NaOBu-t) were added to a 1000 mL single-necked flask, and vacuumized for nitrogen exchange for 3 times, then the reaction was heated up to 90° C. for reacting for 5 h. The reaction was terminated at the end of the reaction. The flask was cooled to room temperature, and reaction liquid was separated, and organic phases were concentrated, then methanol was added and stirred for 1 h, and suction filtration was performed to obtain a faint yellow M8-1.

23 g (50 mmol) M8-1, 12 g (100 mmol) 4-cyclohexyl bromobenzene, 0.9 g (1 mmol) tri(dibenzylidene acetone) dipalladium (namely, Pd₂(dba)₃), 0.5 g tri-tert-butylphosphine ((t-Bu)₃P), 500 mL toluene, and 14.4 g (150 mmol) sodium tert-butoxide (NaOBu-t) were added to a 1000 mL single-necked flask, and vacuumized for nitrogen exchange for 3 times, then the reaction was heated up to 110° C. for reacting for 5 h. The reaction was terminated at the end of the reaction. The flask was cooled to room temperature, and reaction liquid was separated, and organic phases were concentrated, then methanol was added and stirred for 1 h, and suction filtration was performed to obtain a faint yellow powder P173.

M/Z theoretical value: 619; ZAB-HS mass spectrometer (manufactured by Britain Micromass); M/Z measured value: 620.

### Synthesis Example 1-16: Synthesis of the Compound P189

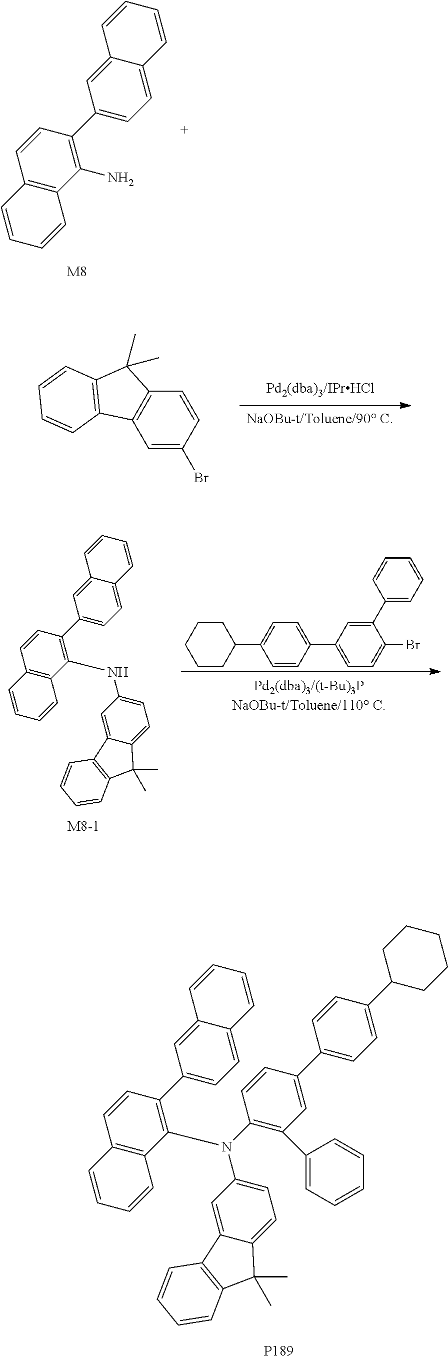

13.5 g (50 mmol) M8, 13.6 g (50 mmol) 3-bromo-9,9-dimethylfluorene, 0.9 g (1 mmol) tri(dibenzylidene acetone) dipalladium (namely, Pd₂(dba)₃), 0.5 g IPr.HCl, 500 mL toluene, and 14.4 g (150 mmol) sodium tert-butoxide (NaOBu-t) were added to a 1000 mL single-necked flask, and vacuumized for nitrogen exchange for 3 times, then the reaction was heated up to 90° C. for reacting for 5 h. The reaction was terminated at the end of the reaction. The flask was cooled to room temperature, and reaction liquid was separated, and organic phases were concentrated, then methanol was added and stirred for 1 h, and suction filtration was performed to obtain a faint yellow M8-1.

23 g (50 mmol) M8-1, 20 g (100 mmol) 2-phenyl-4-(4′-cyclohexyl phenyl) bromobenzene, 0.9 g (1 mmol) tri(dibenzylidene acetone) dipalladium (namely, Pd₂(dba)₃), 0.5 g tri-tert-butylphosphine ((t-Bu)₃P), 500 mL toluene, and 14.4 g (150 mmol) sodium tert-butoxide (NaOBu-t) were added to a 1000 mL single-necked flask, and vacuumized for nitrogen exchange for 3 times, then the reaction was heated up to 110° C. for reacting for 5 h. The reaction was terminated at the end of the reaction. The flask was cooled to room temperature, and reaction liquid was separated, and organic phases were concentrated, then methanol was added and stirred for 1 h, and suction filtration was performed to obtain a faint yellow powder P189.

M/Z theoretical value: 771; ZAB-HS mass spectrometer (manufactured by Britain Micromass); M/Z measured value: 772.

### Synthesis Example 1-17: Synthesis of the Compound P198

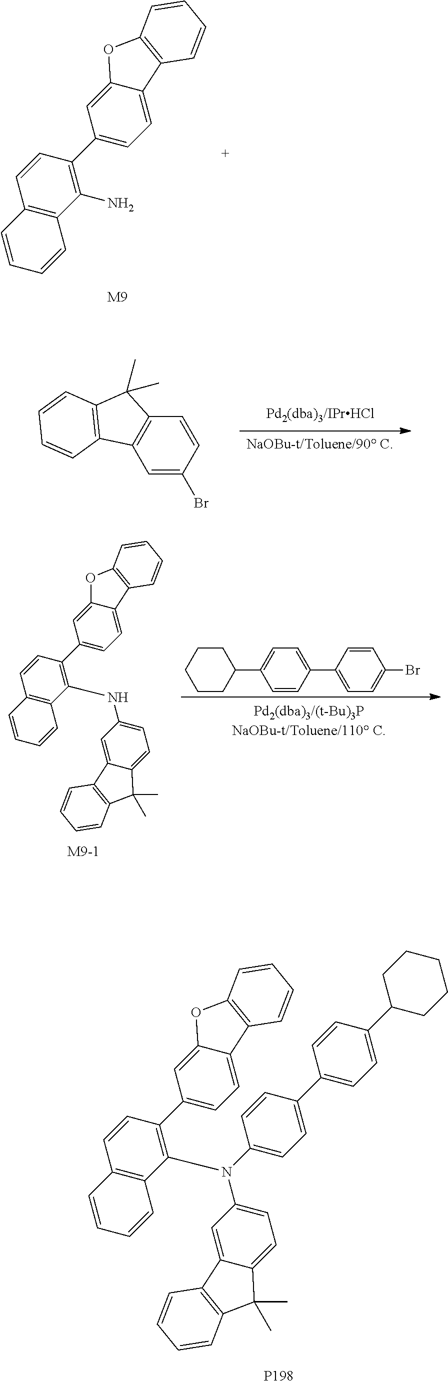

15.5 g (50 mmol) M9, 13.6 g (50 mmol) 3-bromo-9,9-dimethylfluorene, 0.9 g (1 mmol) tri(dibenzylidene acetone) dipalladium (namely, Pd₂(dba)₃), 0.5 g IPr.HCl, 500 mL toluene, and 14.4 g (150 mmol) sodium tert-butoxide (NaOBu-t) were added to a 1000 mL single-necked flask, and vacuumized for nitrogen exchange for 3 times, then the reaction was heated up to 90° C. for reacting for 5 h. The reaction was terminated at the end of the reaction. The flask was cooled to room temperature, and reaction liquid was separated, and organic phases were concentrated, then methanol was added and stirred for 1 h, and suction filtration was performed to obtain a faint yellow M9-1.

25 g (50 mmol) M9-1, 16 g (100 mmol) 4-(4′-cyclohexyl phenyl) bromobenzene, 0.9 g (1 mmol) tri(dibenzylidene acetone) dipalladium (namely, Pd₂(dba)₃), 0.5 g tri-tert-butylphosphine ((t-Bu)₃P), 500 mL toluene, and 14.4 g (150 mmol) sodium tert-butoxide (NaOBu-t) were added to a 1000 mL single-necked flask, and vacuumized for nitrogen exchange for 3 times, then the reaction was heated up to 110° C. for reacting for 5 h. The reaction was terminated at the end of the reaction. The flask was cooled to room temperature, and reaction liquid was separated, and organic phases were concentrated, then methanol was added and stirred for 1 h, and suction filtration was performed to obtain a faint yellow powder P198.

M/Z theoretical value: 735; ZAB-HS mass spectrometer (manufactured by Britain Micromass); M/Z measured value: 736.

### Synthesis Example 1-18: Synthesis of the Compound P209

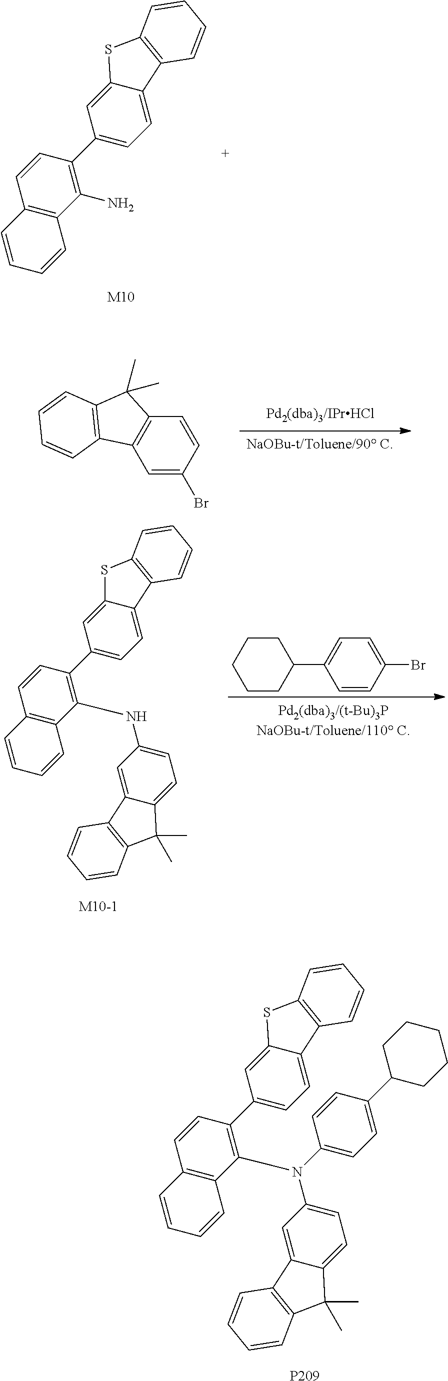

16 g (50 mmol) M10, 13.6 g (50 mmol) 3-bromo-9,9-dimethylfluorene, 0.9 g (1 mmol) tri(dibenzylidene acetone) dipalladium (namely, Pd₂(dba)₃), 0.5 g IPr.HCl, 500 mL toluene, and 14.4 g (150 mmol) sodium tert-butoxide (NaOBu-t) were added to a 1000 mL single-necked flask, and vacuumized for nitrogen exchange for 3 times, then the reaction was heated up to 90° C. for reacting for 5 h. The reaction was terminated at the end of the reaction. The flask was cooled to room temperature, and reaction liquid was separated, and organic phases were concentrated, then methanol was added and stirred for 1 h, and suction filtration was performed to obtain a faint yellow powder M10-1.

26 g (50 mmol) M10-1, 12 g (100 mmol) 4-(4,-cyclohexyl phenyl) bromobenzene, 0.9 g (1 mmol) tri(dibenzylidene acetone) dipalladium (namely, Pd₂(dba)₃), 0.5 g tri-tert-butylphosphine ((t-Bu)₃P), 500 mL methylbenzene, and 14.4 g (150 mmol) sodium tert-butoxide (NaOBu-t) were added to a 1000 mL single-necked flask, and vacuumized for nitrogen exchange for 3 times, then the reaction was heated up to 110° C. for reacting for 5 h. The reaction was terminated at the end of the reaction. The flask was cooled to room temperature, and reaction liquid was separated, and organic phases were concentrated, then methanol was added and stirred for 1 h, and suction filtration was performed to obtain a faint yellow powder P209.

M/Z theoretical value: 675; ZAB-HS mass spectrometer (manufactured by Britain Micromass); M/Z measured value: 676.

### Synthesis Example 1-19: Synthesis of the Compound P224

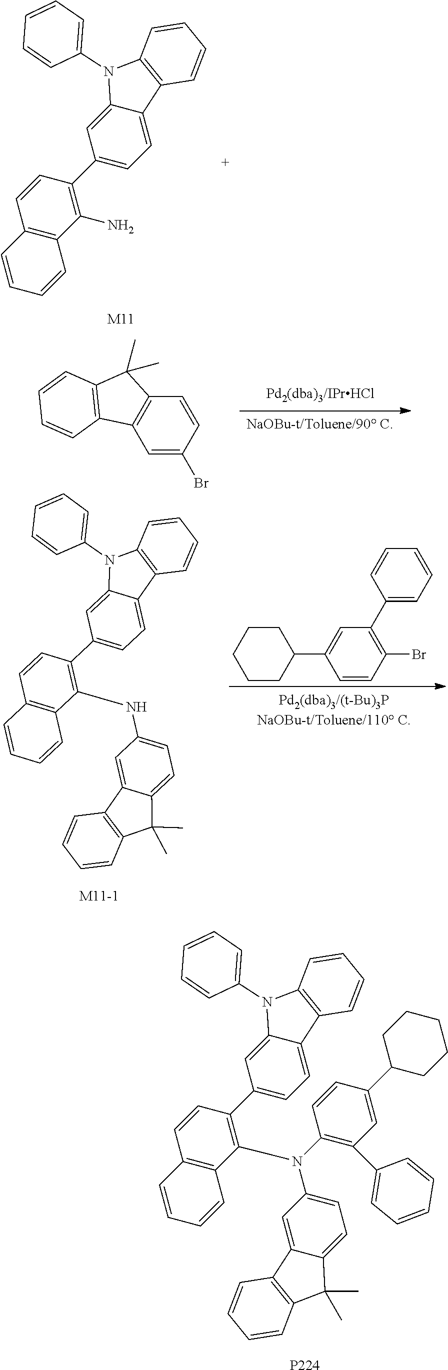

19 g (50 mmol) M11, 13.6 g (50 mmol) 3-bromo-9,9-dimethylfluorene, 0.9 g (1 mmol) tri(dibenzylidene acetone) dipalladium (namely, Pd₂(dba)₃), 0.5 g IPr.HCl, 500 mL toluene, and 14.4 g (150 mmol) sodium tert-butoxide (NaOBu-t) were added to a 1000 mL single-necked flask, and vacuumized for nitrogen exchange for 3 times, then the reaction was heated up to 90° C. for reacting for 5 h. The reaction was terminated at the end of the reaction. The flask was cooled to room temperature, and reaction liquid was separated, and organic phases were concentrated, then methanol was added and stirred for 1 h, and suction filtration was performed to obtain a faint yellow M11-1.

29 g (50 mmol) M11-1, 16 g (100 mmol) 2-phenyl-4-cyclohexyl bromobenzene, 0.9 g (1 mmol) tri(dibenzylidene acetone) dipalladium (namely, Pd₂(dba)₃), 0.5 g tri-tert-butylphosphine ((t-Bu)₃P), 500 mL toluene, and 14.4 g (150 mmol) sodium tert-butoxide (NaOBu-t) were added to a 1000 mL single-necked flask, and vacuumized for nitrogen exchange for 3 times, then the reaction was heated up to 110° C. for reacting for 5 h. The reaction was terminated at the end of the reaction. The flask was cooled to room temperature, and reaction liquid was separated, and organic phases were concentrated, then methanol was added and stirred for 1 h, and suction filtration was performed to obtain a faint yellow powder P224.

M/Z theoretical value: 810; ZAB-HS mass spectrometer (manufactured by Britain Micromass); M/Z measured value: 811.

### Synthesis Example 1-20: Synthesis of the Compound P229

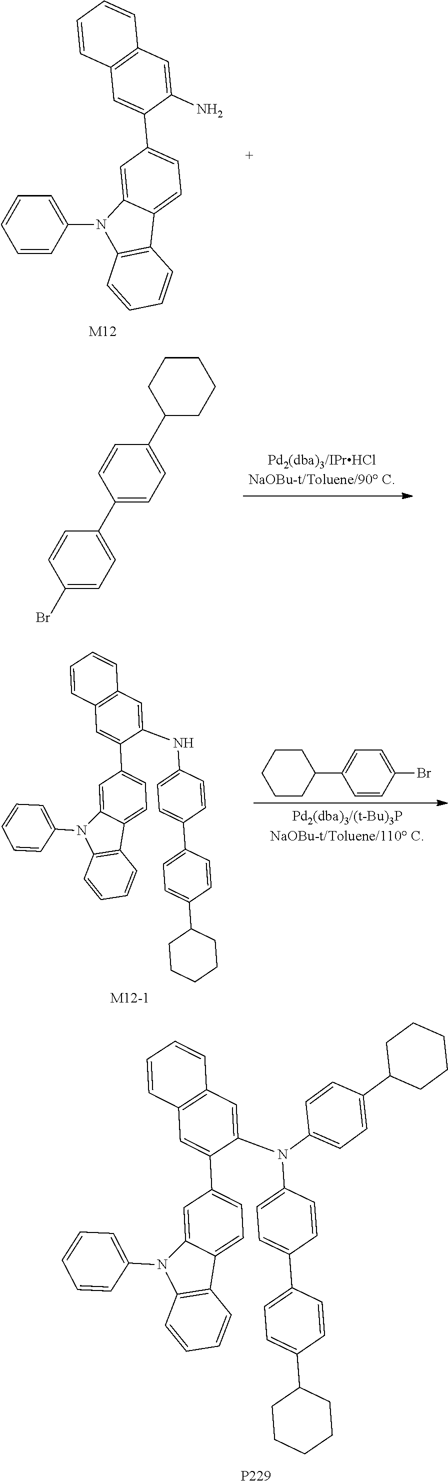

19 g (50 mmol) M12, 16 g (50 mmol) 4-(4-cyclohexyl phenyl) bromobenzene, 0.9 g (1 mmol) tri(dibenzylidene acetone) dipalladium (namely. Pd₂(dba)₃), 0.5 g IPr.HCl, 500 mL toluene, and 14.4 g (150 mmol) sodium tert-butoxide (NaOBu-t) were added to a 1000 mL single-necked flask, and vacuumized for nitrogen exchange for 3 times, then the reaction was heated up to 90° C. for reacting for 5 h. The reaction was terminated at the end of the reaction. The flask was cooled to room temperature, and reaction liquid was separated, and organic phases were concentrated, then methanol was added and stirred for 1 h, and suction filtration was performed to obtain a faint yellow M12-1.

31 g (50 mmol) M12-1, 12 g (100 mmol) 4-cyclohexyl bromobenzene, 0.9 g (1 mmol) tri(dibenzylidene acetone) dipalladium (namely, Pd₂(dba)₃), 0.5 g tri-tert-butylphosphine ((t-Bu)₃P), 500 mL toluene, and 14.4 g (150 mmol) sodium tert-butoxide (NaOBu-t) were added to a 1000 mL single-necked flask, and vacuumized for nitrogen exchange for 3 times, then the reaction was heated up to 110° C. for reacting for 5 h. The reaction was terminated at the end of the reaction. The flask was cooled to room temperature, and reaction liquid was separated, and organic phases were concentrated, then methanol was added and stirred for 1 h, and suction filtration was performed to obtain a faint yellow powder P229.

M/Z theoretical value: 776; ZAB-HS mass spectrometer (manufactured by Britain Micromass); M/Z measured value: 777.

### Synthesis Example 1-21: Synthesis of the Compound P269

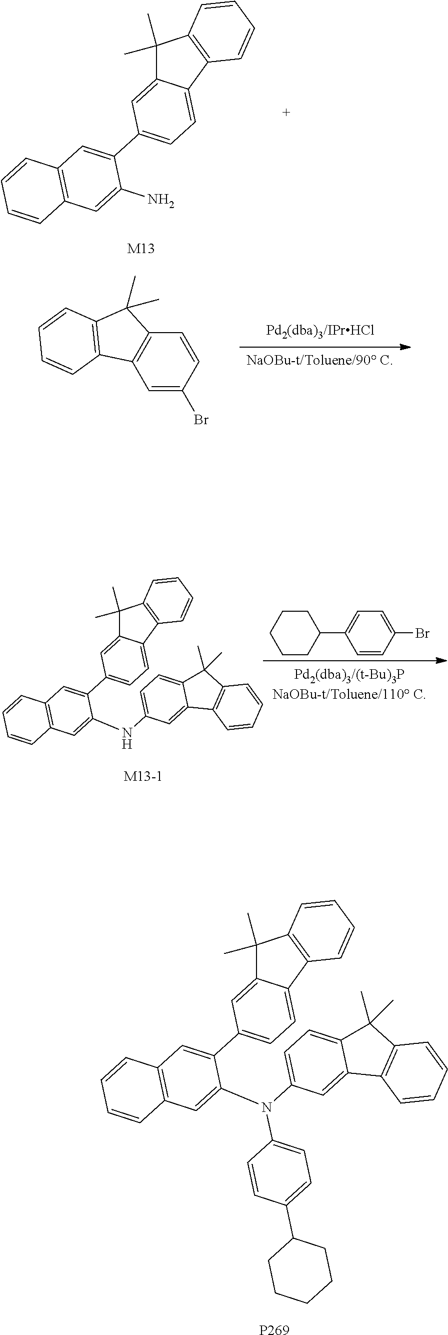

16 g (50 mmol) M13, 13.6 g (50 mmol) 3-bromo-9,9-dimethylfluorene, 0.9 g (1 mmol) tri(dibenzylidene acetone) dipalladium (namely, Pd₂(dba)₃), 0.5 g IPr.HCl, 500 mL toluene, and 14.4 g (150 mmol) sodium tert-butoxide (NaOBu-t) were added to a 1000 mL single-necked flask, and vacuumized for nitrogen exchange for 3 times, then the reaction was heated up to 90° C. for reacting for 5 h. The reaction was terminated at the end of the reaction. The flask was cooled to room temperature, and reaction liquid was separated, and organic phases were concentrated, then methanol was added and stirred for 1 h, and suction filtration was performed to obtain a faint yellow powder M13-1.

26.5 g (50 mmol) M13-1, 12 g (100 mmol) 4-(4,-cyclohexyl phenyl) bromobenzene, 0.9 g (1 mmol) tri(dibenzylidene acetone) dipalladium (namely, Pd₂(dba)₃), 0.5 g tri-tert-butylphosphine ((t-Bu)₃P), 500 mL toluene, and 14.4 g (150 mmol) sodium tert-butoxide (NaOBu-t) were added to a 1000 mL single-necked flask, and vacuumized for nitrogen exchange for 3 times, then the reaction was heated up to 110° C. for reacting for 5 h. The reaction was terminated at the end of the reaction. The flask was cooled to room temperature, and reaction liquid was separated, and organic phases were concentrated, then methanol was added and stirred for 1 h, and suction filtration was performed to obtain a faint yellow powder P269.

M/Z theoretical value: 685; ZAB-HS mass spectrometer (manufactured by Britain Micromass); M/Z measured value: 686.

### Synthesis Example 1-22: Synthesis of the Compound P179

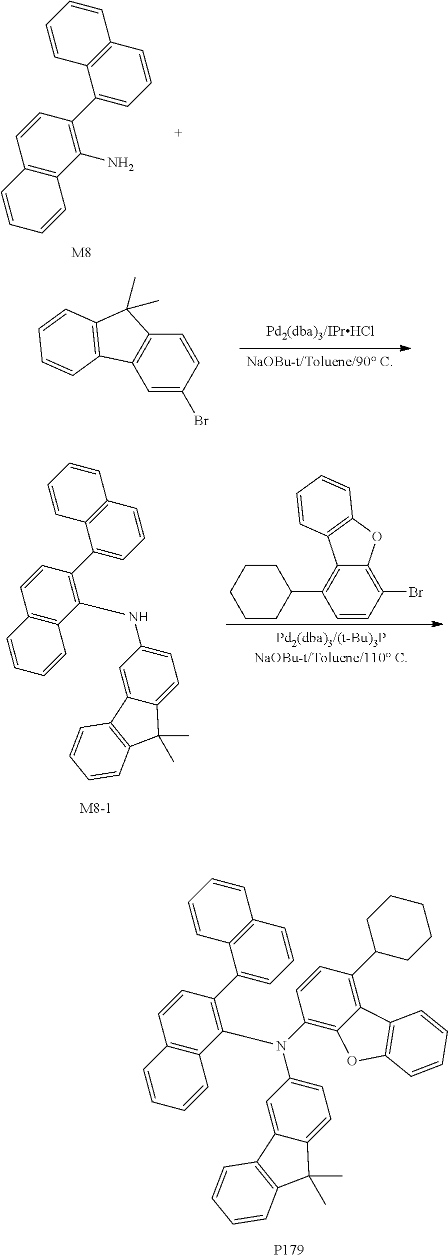

13.5 g (50 mmol) M8, 13.6 g (50 mmol) 3-bromo-9,9-dimethylfluorene, 0.9 g (1 mmol) tri(dibenzylidene acetone) dipalladium (namely, Pd₂(dba)₃), 0.5 g IPr.HCl, 500 mL toluene, and 14.4 g (150 mmol) sodium tert-butoxide (NaOBu-t) were added to a 1000 mL single-necked flask, and vacuumized for nitrogen exchange for 3 times, then the reaction was heated up to 90° C. for reacting for 5 h. The reaction was terminated at the end of the reaction. The flask was cooled to room temperature, and reaction liquid was separated, and organic phases were concentrated, then methanol was added and stirred for 1 h, and suction filtration was performed to obtain a faint yellow M8-1.

23 g (50 mmol) M8-1, 16.5 g (100 mmol) 1-cyclohexyl-4-bromodibenzofuran, 0.9 g (1 mmol) tri(dibenzylidene acetone) dipalladium (namely, Pd₂(dba)₃), 0.5 g tri-tert-butylphosphine ((t-Bu)₃P), 500 mL toluene, and 14.4 g (150 mmol) sodium tert-butoxide (NaOBu-t) were added to a 1000 mL single-necked flask, and vacuumized for nitrogen exchange for 3 times, then the reaction was heated up to 110° C. for reacting for 5 h. The reaction was terminated at the end of the reaction. The flask was cooled to room temperature, and reaction liquid was separated, and organic phases were concentrated, then methanol was added and stirred for 1 h, and suction filtration was performed to obtain a faint yellow powder P179.

M/Z theoretical value: 709; ZAB-HS mass spectrometer (manufactured by Britain Micromass); M/Z measured value: 710.

### Synthesis Example 1-23: Synthesis of the Compound P287

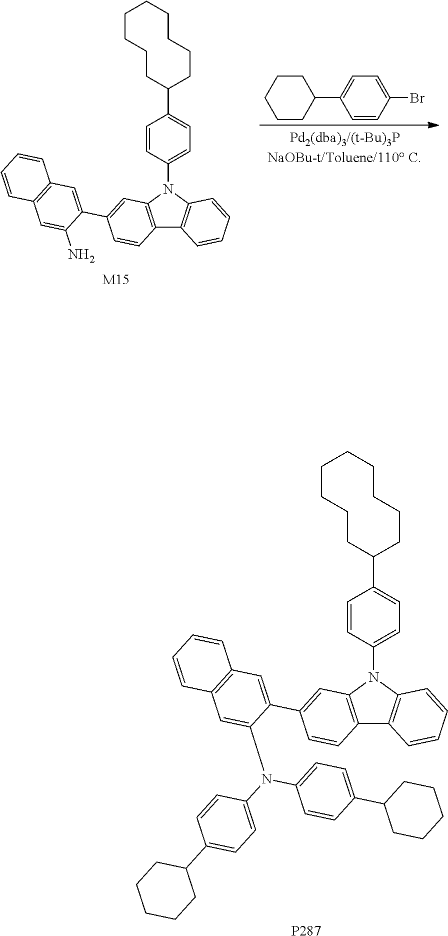

26 g (50 mmol) M15, 24 g (100 mmol) 4-cyclohexyl bromobenzene, 0.9 g (1 mmol) tri(dibenzylidene acetone) dipalladium (namely, Pd₂(dba)₃), 0.5 g tri-tert-butylphosphine ((t-Bu)₃P), 500 mL toluene, and 14.4 g (150 mmol) sodium tert-butoxide (NaOBu-t) were added to a 1000 mL single-necked flask, and vacuumized for nitrogen exchange for 3 times, then the reaction was heated up to 110° C. for reacting for 5 h. The reaction was terminated at the end of the reaction. The flask was cooled to room temperature, and reaction liquid was separated, and organic phases were concentrated, then methanol was added and stirred for 1 h, and suction filtration was performed to obtain a faint yellow powder P287.

M/Z theoretical value: 839; ZAB-HS mass spectrometer (manufactured by Britain Micromass); M/Z measured value: 840.

### Synthesis Example 1-24: Synthesis of the Compound P42

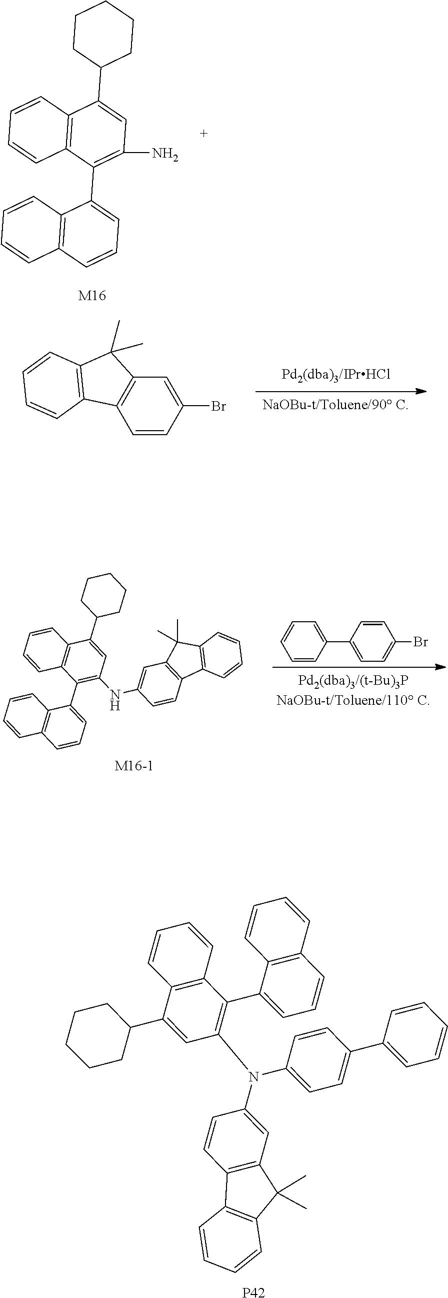

17 g (50 mmol) M16, 13.6 g (50 mmol) 3-bromo-9,9-dimethylfluorene, 0.9 g (1 mmol) tri(dibenzylidene acetone) dipalladium (namely, Pd₂(dba)₃), 0.5 g IPr.HCl, 500 mL toluene, and 14.4 g (150 mmol) sodium tert-butoxide (NaOBu-t) were added to a 1000 mL single-necked flask, and vacuumized for nitrogen exchange for 3 times, then the reaction was heated up to 90° C. for reacting for 5 h. The reaction was terminated at the end of the reaction. The flask was cooled to room temperature, and reaction liquid was separated, and organic phases were concentrated, then methanol was added and stirred for 1 h, and suction filtration was performed to obtain a faint yellow powder M16-1.

27 g (50 mmol) M16-1, 12 g (50 mmol) 4-bromobiphenyl, 0.9 g (1 mmol) tri(dibenzylidene acetone) dipalladium (namely, Pd₂(dba)₃), 0.5 g tri-tert-butylphosphine ((t-Bu)₃P), 500 mL toluene, and 14.4 g (150 mmol) sodium tert-butoxide (NaOBu-t) were added to a 1000 mL single-necked flask, and vacuumized for nitrogen exchange for 3 times, then the reaction was heated up to 110° C. for reacting for 5 h. The reaction was terminated at the end of the reaction. The flask was cooled to room temperature, and reaction liquid was separated, and organic phases were concentrated, then methanol was added and stirred for 1 h, and suction filtration was performed to obtain a faint yellow powder P42.

M/Z theoretical value: 695; ZAB-HS mass spectrometer (manufactured by Britain Micromass); M/Z measured value: 696.

### Synthesis Example 1-25: Synthesis of the Compound P278

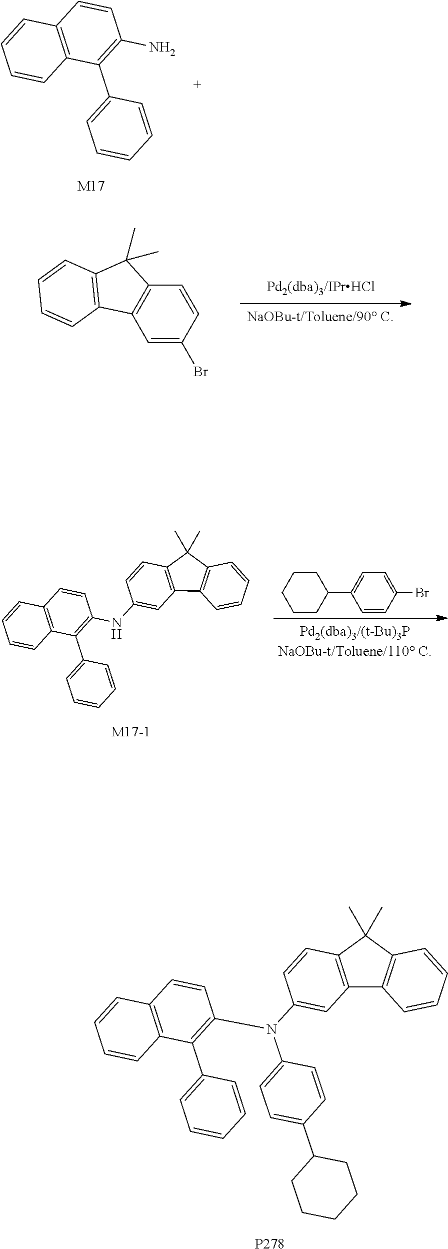

11 g (50 mmol) M17, 13.6 g (50 mmol) 3-bromo-9,9-dimethylfluorene, 0.9 g (1 mmol) tri(dibenzylidene acetone) dipalladium (namely, Pd₂(dba)₃), 0.5 g IPr.HCl, 500 mL toluene, and 14.4 g (150 mmol) sodium tert-butoxide (NaOBu-t) were added to a 1000 mL single-necked flask, and vacuumized for nitrogen exchange for 3 times, then the reaction was heated up to 90° C. for reacting for 5 h. The reaction was terminated at the end of the reaction. The flask was cooled to room temperature, and reaction liquid was separated, and organic phases were concentrated, then methanol was added and stirred for 1 h, and suction filtration was performed to obtain a faint yellow powder M17-1.

21 g (50 mmol) M17-1, 12 g (50 mmol) 4-cyclohexyl bromobenzene, 0.9 g (1 mmol) tri(dibenzylidene acetone) dipalladium (namely, Pd₂(dba)₃), 0.5 g tri-tert-butylphosphine ((t-Bu)₃P), 500 mL toluene, and 14.4 g (150 mmol) sodium tert-butoxide (NaOBu-t) were added to a 1000 mL single-necked flask, and vacuumized for nitrogen exchange for 3 times, then the reaction was heated up to 110° C. for reacting for 5 h. The reaction was terminated at the end of the reaction. The flask was cooled to room temperature, and reaction liquid was separated, and organic phases were concentrated, then methanol was added and stirred for 1 h, and suction filtration was performed to obtain a faint yellow powder P278.

M/Z theoretical value: 569; ZAB-HS mass spectrometer (manufactured by Britain Micromass); M/Z measured value: 570.

### Synthesis of the Compounds of Preferred Embodiment II

In this present invention, the synthetic method of the compound is described briefly, and the representative synthetic route of the compound is as follows:

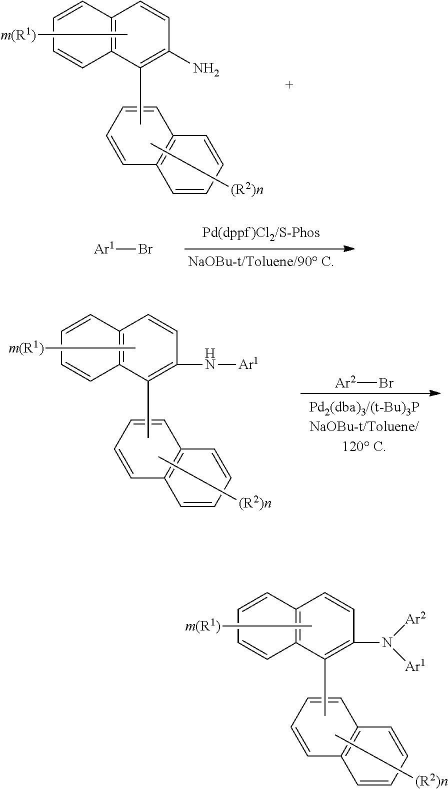

Based on the synthetic route and idea of the above compound, a person skilled in the art may obtain a compound having substituents of Ar¹, Ar², R¹ and R².

### Synthesis Example 2-1: Synthesis of the Compound N1

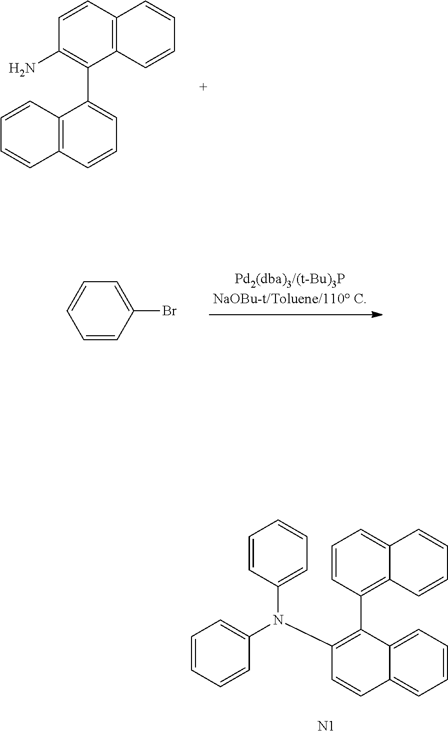 [SMILES: Nc1ccc2ccccc2c1-c1cccc2ccccc12 ; Brc1ccccc1 ; c1ccc(N(c2ccccc2)c2ccc3ccccc3c2-c2cccc3ccccc23)cc1 ; "N1"]

13.5 g (50 mmol) 2-amino-1,1′-dinaphthalene, 15.7 g (100 mmol) bromobenzene, 0.9 g (1 mmol) tri(dibenzylidene acetone) dipalladium (namely, Pd₂(dba)₃), 0.5 mL tri-tert-butylphosphine, 500 mL toluene, and 14.4 g (150 mmol) sodium tert-butoxide (NaOBu-t) were added to a 1000 mL single-necked flask, and vacuumized for nitrogen exchange for 3 times, then the reaction was heated up to 110° C. for reacting for 5 h. The reaction was terminated at the end of the reaction. The flask was cooled to room temperature, and reaction liquid was separated, and organic phases were concentrated, then methanol was added and stirred for 1 h, and suction filtration was performed to obtain a faint yellow powder N1; M/Z theoretical value: 421, and M/Z measured value: 422.

### Synthesis Example 2-2: Synthesis of the Compound N13

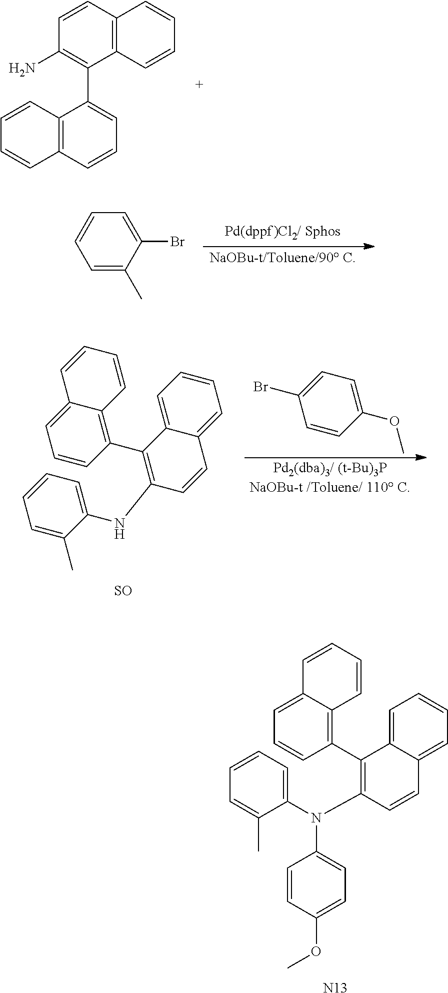

13.5 g (50 mmol) 2-amino-1,1′-dinaphthalene, 8.5 g (50 mmol) 2-methylbromobenzene, 0.7 g (1 mmol) [1,1′-bis(diphenylphosphine)ferrocene] palladium dichloride (Pd(dppf)Cl₂), 0.5 g 2-bicyclohexylphosphine-2′,6′-dimethoxybiphenyl (Sphos), 500 mL toluene, and 14.4 g (150 mmol) sodium tert-butoxide were added to a 1000 mL single-necked flask, and vacuumized for nitrogen exchange for 3 times, then the reaction was heated up to 90° C. for reacting for 5 h. The reaction was terminated at the end of the reaction. The flask was cooled to room temperature, and reaction liquid was separated, and organic phases were concentrated, then methanol was added and stirred for 1 h, and suction filtration was performed to obtain a faint yellow powder S0.

18 g (50 mmol) S0, 9.5 g (50 mmol) p-bromophenyl methyl ether, 0.9 g (1 mmol) tri(dibenzylidene acetone) dipalladium, 500 mL toluene were added to a 1000 mL single-necked flask, and vacuumized for nitrogen exchange for 3 times, and 0.5 mL tri-tert-butylphosphine (P(t-Bu)₃) toluene solution was added, and heated up to 110° C. for reaction for 12 h; at the end of the reaction; solvent was removed by evaporation, and silica-gel column chromatography was performed to obtain N13; M/Z theoretical value: 465, M/Z measured value: 466.

### Synthesis Example 2-3: Synthesis of the Compound N34

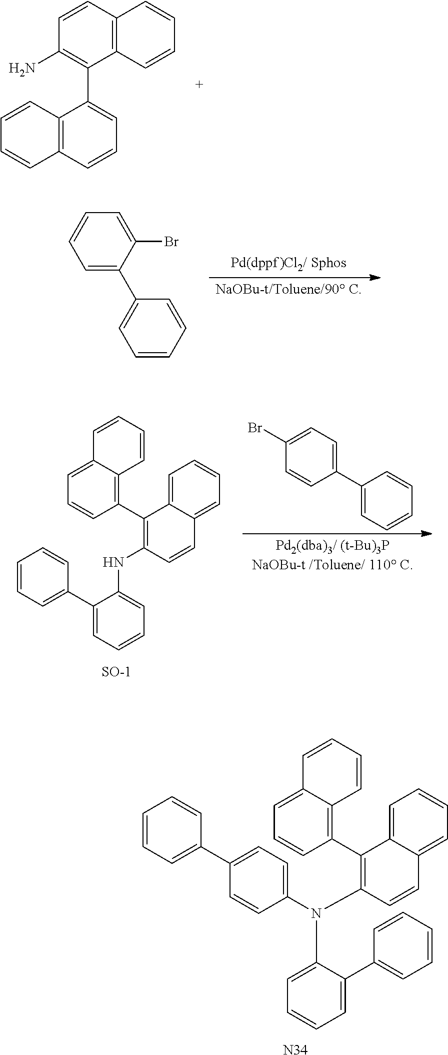

13.5 g (50 mmol) 2-amino-1,1′-dinaphthalene, 12 g (50 mmol) 2-bromobiphenyl, 0.7 g (1 mmol) [1,1′-bis(diphenylphosphine)ferrocene] palladium dichloride (Pd(dppf)Cl₂), 2-bicyclohexylphosphine-2′,6′-dimethoxybiphenyl, 500 mL toluene, and 14.4 g (150 mmol) sodium tert-butoxide were added to a 1000 mL single-necked flask, and vacuumized for nitrogen exchange for 3 times, then the reaction was heated up to 90° C. for reacting for 5 h. The reaction was terminated at the end of the reaction. The flask was cooled to room temperature, and reaction liquid was separated, and organic phases were concentrated, then methanol was added and stirred for 1 h, and suction filtration was performed to obtain a faint yellow powder S0-1.

21 g (50 mmol) S0-1, 12 g (50 mmol) p-bromobiphenyl, 0.9 g (1 mmol) tri(dibenzylidene acetone) dipalladium, 500 mL toluene were added to a 1000 mL single-necked flask, and vacuumized for nitrogen exchange for 3 times, and 0.5 mL tri-tert-butylphosphine (P(t-Bu)₃) toluene solution was added, and heated up to 110° C. for reaction for 12 h; at the end of the reaction, solvent was removed by evaporation, and silica-gel column chromatography was performed to obtain N34; M/Z theoretical value: 573, M/Z measured value: 574.

### Synthesis Example 2-4: Synthesis of the Compound N63

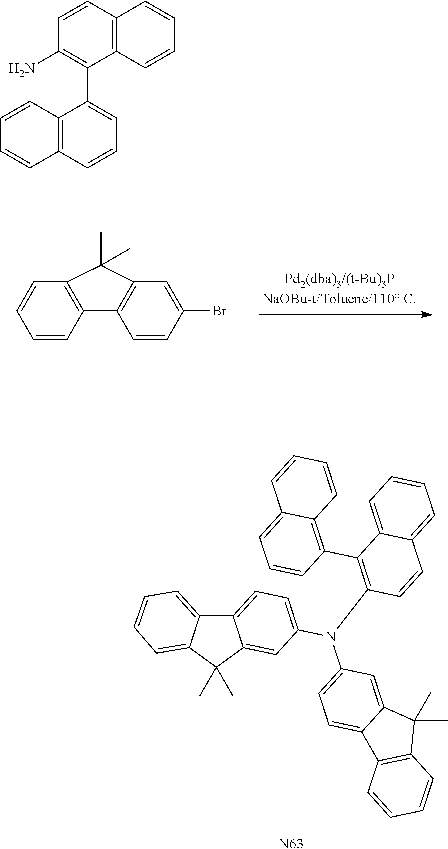 [SMILES: Nc1ccc2ccccc2c1-c1cccc2ccccc12 ; CC1(C)c2ccccc2-c2ccc(Br)cc21 ; CC1(C)c2ccccc2-c2ccc(N(c3ccc4c(c3)C(C)(C)c3ccccc3-4)c3ccc4ccccc4c3-c3cccc4ccccc34)cc21 ; "N63"]

13.5 g (50 mmol) 2-amino-1,1′-dinaphthalene, 27 g (100 mmol) 2-bromo-9,9′-dimethylfluorene, 0.9 g (1 mL) tri(dibenzylidene acetone) dipalladium (namely, Pd₂(dba)₃), 0.5 mL tri-tert-butylphosphine (P(t-Bu)₃), 500 mL toluene, and 14.4 g (150 mmol) sodium tert-butoxide (NaOBu-t) were added to a 1000 mL single-necked flask, and vacuumized for nitrogen exchange for 3 times, then the reaction was heated up to 110° C. for reacting for 5 h. The reaction was terminated at the end of the reaction. The flask was cooled to room temperature, and reaction liquid was separated, and organic phases were concentrated, then methanol was added and stirred for 1 h, and suction filtration was performed to obtain a faint yellow powder N63; M/Z theoretical value: 653, and M/Z measured value: 654.

### Synthesis Example 2-5: Synthesis of the Compound N93

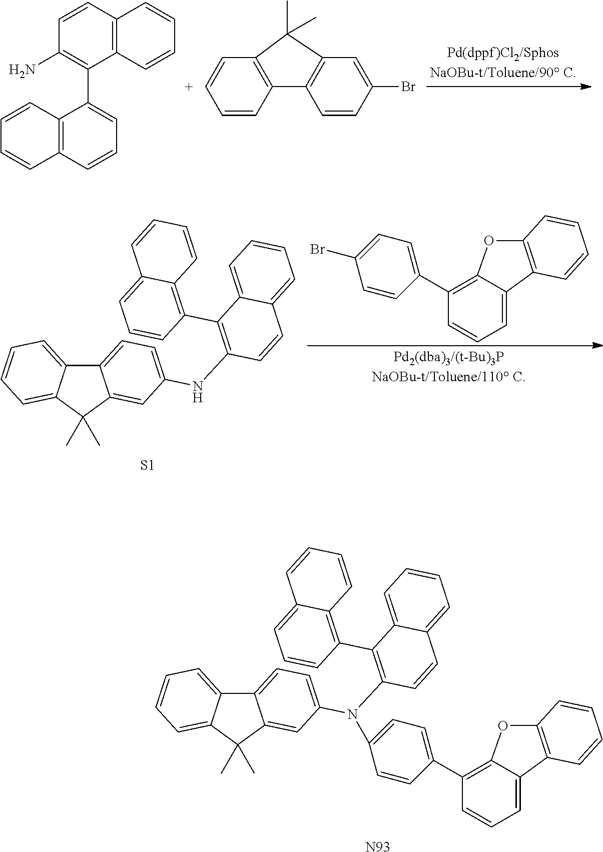

13.5 g (50 mmol) 2-amino-1,1′-dinaphthalene, 13.5 g (50 mmol) 2-bromo-9,9′-dimethylfluorene, 0.7 g (1 mmol) [1,1′-bis(diphenylphosphine)ferrocene] palladium dichloride (Pd(dppf)Cl₂), 0.5 g 2-bicyclohexylphosphine-2′,6′-dimethoxybiphenyl, 500 mL toluene, and 14.4 g (150 mmol) sodium tert-butoxide were added to a 1000 mL single-necked flask, and vacuumized for nitrogen exchange for 3 times, then the reaction was heated up to 90° C. for reacting for 5 h. The reaction was terminated at the end of the reaction. The flask was cooled to room temperature, and reaction liquid was separated, and organic phases were concentrated, then methanol was added and stirred for 1 h, and suction filtration was performed to obtain a faint yellow powder S1.

23 g (50 mmol) Si, 16.1 g (50 mmol) 4-(4-bromo-phenyl)-dibenzofuran, 0.9 g (1 mmol) tri(dibenzylidene acetone) dipalladium, 500 mL toluene were added to a 1000 mL single-necked flask, and vacuumized for nitrogen exchange for 3 times, and 0.5 mL tri-tert-butylphosphine (P(t-Bu)₃) toluene solution was added, and heated up to 110° C. for reaction for 12 h; at the end of the reaction, solvent was removed by evaporation, and silica-gel column chromatography was performed to obtain N93; M/Z theoretical value: 703, M/Z measured value: 704.

### Synthesis Example 2-6: Synthesis of the Compound N94

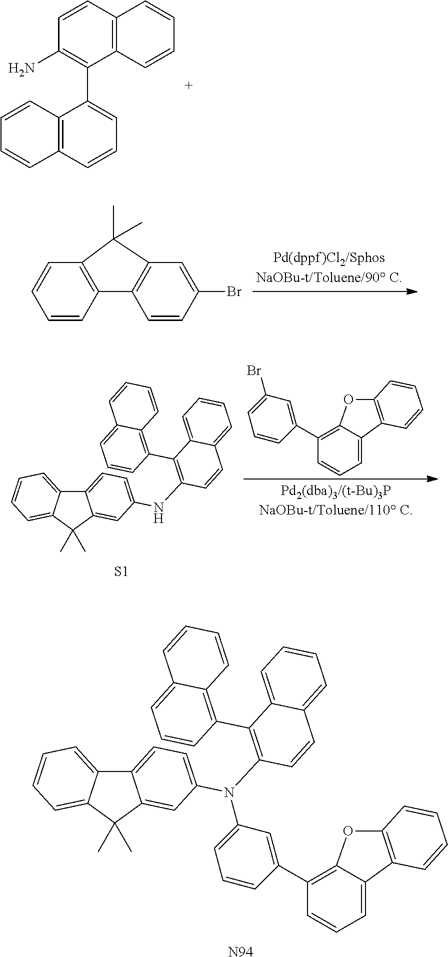

13.5 g (50 mmol) 2-amino-1,1′-dinaphthalene, 13.5 g (50 mmol) 2-bromo-9,9′-dimethyfluorene, 0.7 g (1 mmol) [1,1′-bis(diphenylphosphine)ferrocene] palladium dichloride, 0.5 g 2-bicyclohexylphosphine-2′,6′-dimethoxybiphenyl, 500 mL toluene, and 14.4 g (150 mmol) sodium tert-butoxide were added to a 1000 mL single-necked flask, and vacuumized for nitrogen exchange for 3 times, then the reaction was heated up to 90° C. for reacting for 5 h. The reaction was terminated at the end of the reaction. The flask was cooled to room temperature, and reaction liquid was separated, and organic phases were concentrated, then methanol was added and stirred for 1 h, and suction filtration was performed to obtain a faint yellow powder S1.

23 g (50 mmol) S1, 16.1 g (50 mmol) 3-(4-bromo-phenyl)-dibenzofuran, 0.9 g (1 mmol) tri(dibenzylidene acetone) dipalladium, 500 mL toluene were added to a 1000 mL single-necked flask, and vacuumized for nitrogen exchange for 3 times, and 0.5 mL tri-tert-butylphosphine toluene solution was added, and heated up to 110° C. for reaction for 12 h; at the end of the reaction, solvent was removed by evaporation, and silica-gel column chromatography was performed to obtain N94; M/Z theoretical value: 703, M/Z measured value: 704.

### Synthesis Example 2-7: Synthesis of the Compound N100

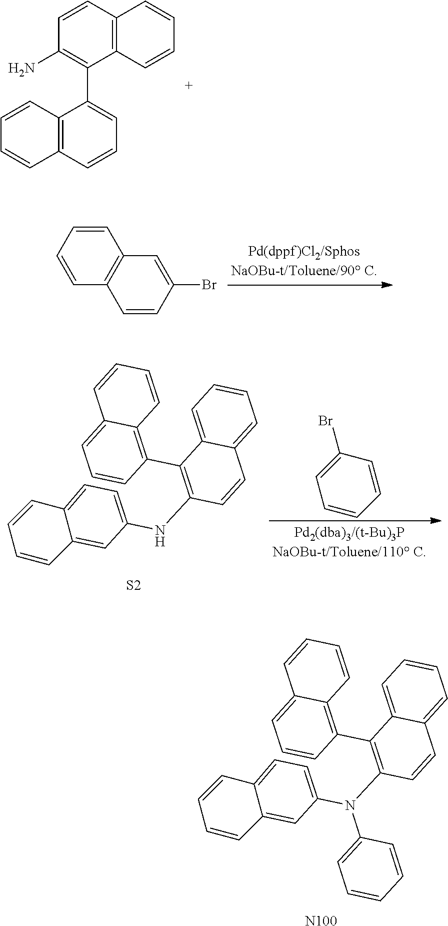

13.5 g (50 mmol) 2-amino-1,1′-dinaphthalene, 10.3 g (50 mmol) 2-bromonaphthalene, 0.7 g (1 mmol) [1,1′-bis(diphenylphosphine)ferrocene] palladium dichloride, 0.5 g 2-bicyclohexylphosphine-2′,6′-dimethoxybiphenyl, 500 mL toluene, and 14.4 g (150 mmol) sodium tert-butoxide were added to a 1000 mL single-necked flask, and vacuumized for nitrogen exchange for 3 times, then the reaction was heated up to 90° C. for reacting for 5 h. The reaction was terminated at the end of the reaction. The flask was cooled to room temperature, and reaction liquid was separated, and organic phases were concentrated, then methanol was added and stirred for 1 h, and suction filtration was performed to obtain a faint yellow powder S2.

23 g (50 mmol) S2, 8.3 g (50 mmol) bromobenzene, 0.9 g (1 mmol) tri(dibenzylidene acetone) dipalladium, 500 mL toluene were added to a 1000 mL single-necked flask, and vacuumized for nitrogen exchange for 3 times, and 0.5 mL tri-tert-butylphosphine toluene solution was added, and heated up to 110° C. for reaction for 12 h; at the end of the reaction, the reaction was terminated. Solvent was removed by evaporation, and silica-gel column chromatography was performed to obtain N100; M/Z theoretical value: 471. M/Z measured value: 472.

### Synthesis Example 2-7: Synthesis of the Compound N120

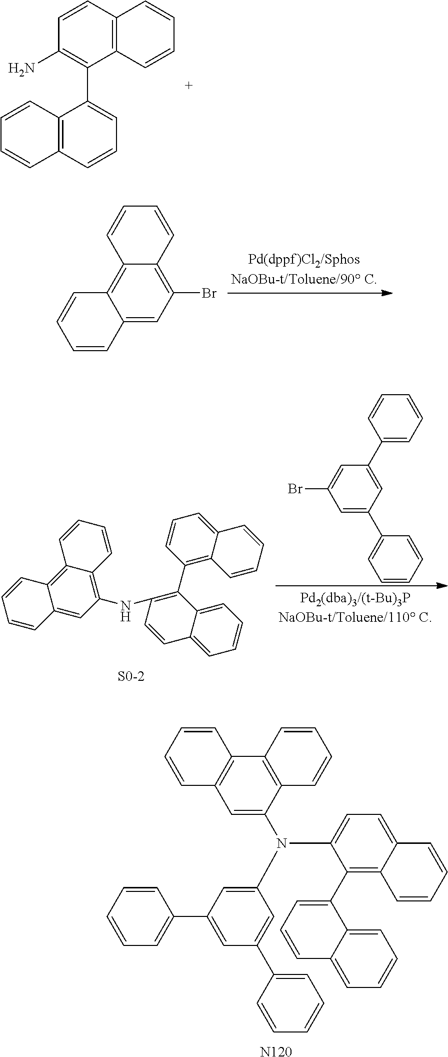 [SMILES: Nc1ccc2ccccc2c1-c1cccc2ccccc12 ; Brc1cc2ccccc2c2ccccc12 ; c1ccc2c(-c3c(Nc4cc5ccccc5c5ccccc45)ccc4ccccc34)cccc2c1 ; Brc1cc(-c2ccccc2)cc(-c2ccccc2)c1 ; c1ccc(-c2cc(-c3ccccc3)cc(N(c3ccc4ccccc4c3-c3cccc4ccccc34)c3cc4ccccc4c4ccccc34)c2)cc1 ; "N120"]

13.5 g (50 mmol) 2-amino-1,1′-dinaphthalene, 13 g (50 mmol) 9-bromophenanthrene, 0.7 g (1 mmol) [1,1′-bis(diphenylphosphine)ferrocene] palladium dichloride (Pd(dppf)Cl₂), 0.5 g 2-bicyclohexylphosphine-2′,6′-dimethoxybiphenyl, 500 mL toluene, and 14.4 g (150 mmol) sodium tert-butoxide were added to a 1000 mL single-necked flask, and vacuumized for nitrogen exchange for 3 times, then the reaction was heated up to 90° C. for reacting for 5 h. The reaction was terminated at the end of the reaction. The flask was cooled to room temperature, and reaction liquid was separated, and organic phases were concentrated, then methanol was added and stirred for 1 h, and suction filtration was performed to obtain a faint yellow powder S0-2.

22 g (50 mmol) S0-2, 15 g (50 mmol) 3,5-diphenylbromobenzene, 0.9 g (1 mmol) tri(dibenzylidene acetone) dipalladium, 500 mL toluene were added to a 1000 mL single-necked flask, and vacuumized for nitrogen exchange for 3 times, and 0.5 mL tri-tert-butylphosphine (P(t-Bu)₃) toluene solution was added, and heated up to 110° C. for reaction for 12 h; at the end of the reaction, solvent was removed by evaporation, and silica-gel column chromatography was performed to obtain N120; M/Z theoretical value: 673, M/Z measured value: 674.

### Synthesis Example 2-9: Synthesis of the Compound N134

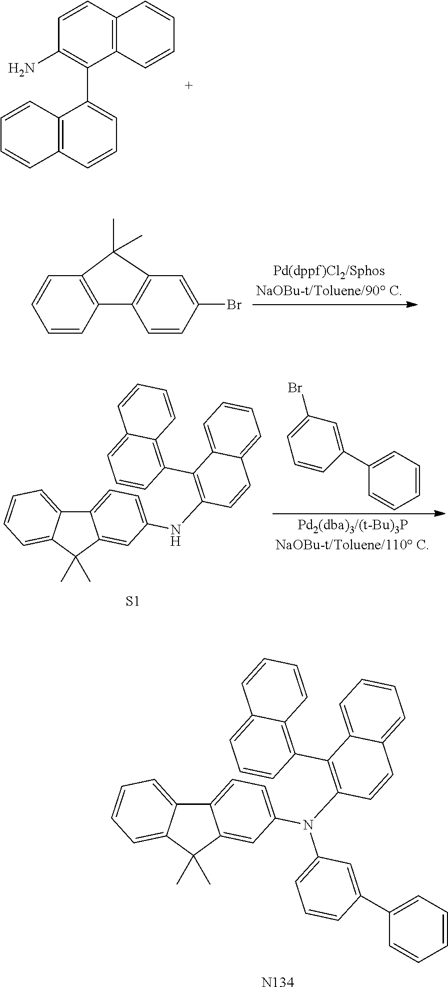

13.5 g (50 mmol) 2-amino-1,1′-dinaphthalene, 13.5 g (50 mmol) 2-bromo-9,9′-dimethylfluorene, 0.7 g (1 mmol) [1,1′-bis(diphenylphosphine)ferrocene] palladium dichloride, 0.5 g 2-bicyclohexylphosphine-2′,6′-dimethoxybiphenyl, 500 mL toluene, and 14.4 g (150 mmol) sodium tert-butoxide were added to a 1000 mL single-necked flask, and vacuumized for nitrogen exchange for 3 times, then the reaction was heated up to 90° C. for reacting for 5 h. The reaction was terminated at the end of the reaction. The flask was cooled to room temperature, and reaction liquid was separated, and organic phases were concentrated, then methanol was added and stirred for 1 h, and suction filtration was performed to obtain a faint yellow powder S1.

23 g (50 mmol) S1, 11.5 g (50 mmol) 3-bromo-biphenyl, 0.9 g (1 mmol) tri(dibenzylidene acetone) dipalladium, 500 mL toluene were added to a 1000 mL single-necked flask, and vacuumized for nitrogen exchange for 3 times, and 0.5 mL tri-tert-butylphosphine toluene solution was added, and heated up to 110° C. for reaction for 12 h; at the end of the reaction, solvent was removed by evaporation, and silica-gel column chromatography was performed to obtain N134; M/Z theoretical value: 613, M/Z measured value: 614.

### Synthesis Example 2-10: Synthesis of the Compound N147

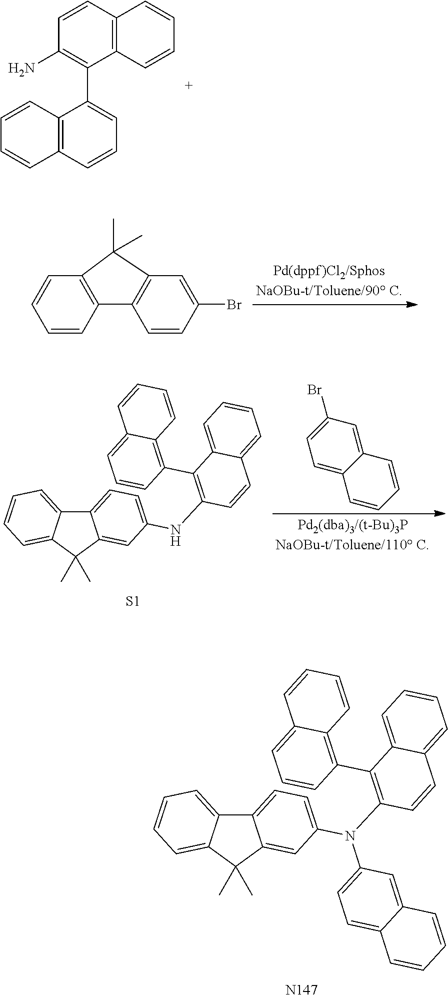

13.5 g (50 mmol) 2-amino-1,1′-dinaphthalene, 13.5 g (50 mmol) 2-bromo-9,9′-dimethylfluorene, 0.7 g (1 mmol) [1,1′-bis(diphenylphosphine)ferrocene] palladium dichloride, 0.5 g 2-bicyclohexylphosphine-2′,6′-dimethoxybiphenyl, 500 mL toluene, and 14.4 g (150 mmol) sodium tert-butoxide were added to a 1000 mL single-necked flask, and vacuumized for nitrogen exchange for 3 times, then the reaction was heated up to 90° C. for reacting for 5 h. The reaction was terminated at the end of the reaction. The flask was cooled to room temperature, and reaction liquid was separated, and organic phases were concentrated, then methanol was added and stirred for 1 h, and suction filtration was performed to obtain a faint yellow powder S1.

23 g (50 mmol) S1, 10.4 g (50 mmol) 2-bromonaphthalene, 0.9 g (1 mmol) tri(dibenzylidene acetone) dipalladium, 500 mL toluene were added to a 1000 mL single-necked flask, and vacuumized for nitrogen exchange for 3 times, and 0.5 mL tri-tert-butylphosphine toluene solution was added, and heated up to 110° C. for reaction for 12 h; at the end of the reaction, solvent was removed by evaporation, and silica-gel column chromatography was performed to obtain N147; M/Z theoretical value: 587, M/Z measured value: 588.

### Synthesis Example 2-11: Synthesis of the Compound N170

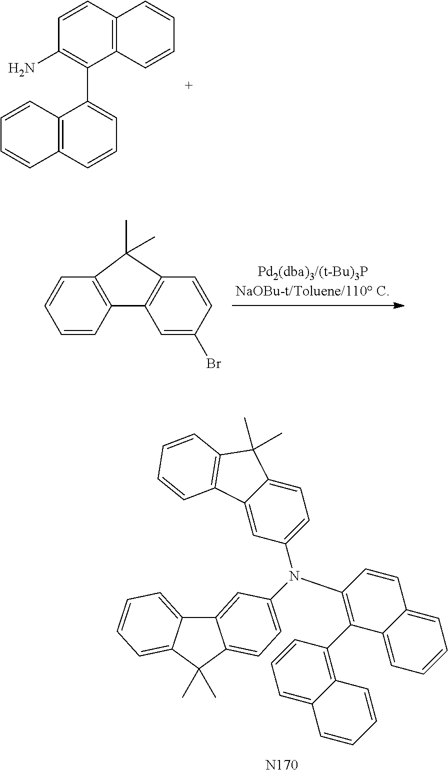 [SMILES: Nc1ccc2ccccc2c1-c1cccc2ccccc12 ; CC1(C)c2ccccc2-c2cc(Br)ccc21 ; CC1(C)c2ccccc2-c2cc(N(c3ccc4c(c3)-c3ccccc3C4(C)C)c3ccc4ccccc4c3-c3cccc4ccccc34)ccc21 ; "N170"]

13.5 g (50 mmol) 2-amino-1,1′-dinaphthalene, 27 g (100 mmol) 3-bromo-9,9′-dimethylfluorene, 0.9 g (1 mmol) tri(dibenzylidene acetone)dipalladium, 0.5 g 2-bicyclohexylphosphine-2′,6′-dimethoxybiphenyl, 500 mL toluene, and 14.4 g (150 mmol) sodium tert-butoxide were added to a 1000 mL single-necked flask, and vacuumized for nitrogen exchange for 3 times, then the reaction was heated up to 110° C. for reacting for 5 h. The reaction was terminated at the end of the reaction. The flask was cooled to room temperature, and reaction liquid was separated, and organic phases were concentrated, then methanol was added and stirred for 1 h, and suction filtration was performed to obtain a faint yellow powder N170; M/Z theoretical value: 653, and M/Z measured value: 654.

### Synthesis Example 2-12: Synthesis of the Compound N176

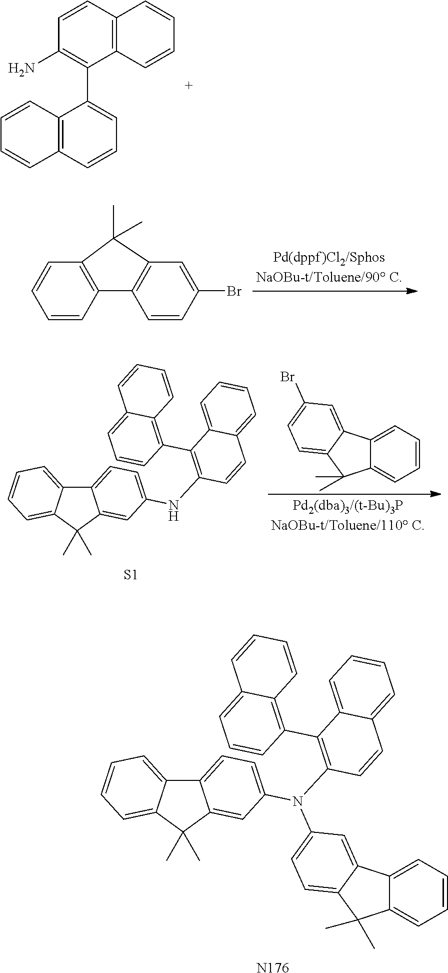

13.5 g (50 mmol) 2-amino-1,1′-dinaphthalene, 13.5 g (50 mmol) 2-bromo-9,9′-dimethylfluorene, 0.7 g (1 mmol) [1,1′-bis(diphenylphosphine)ferrocene] palladium dichloride, 0.5 g 2-bicyclohexylphosphine-2′,6′-dimethoxybiphenyl, 500 mL toluene, and 14.4 g (150 mmol) sodium tert-butoxide were added to a 1000 mL single-necked flask, and vacuumized for nitrogen exchange for 3 times, then the reaction was heated up to 90° C. for reacting for 5 h. The reaction was terminated at the end of the reaction. The flask was cooled to room temperature, and reaction liquid was separated, and organic phases were concentrated, then methanol was added and stirred for 1 h, and suction filtration was performed to obtain a faint yellow powder S1.

23 g (50 mmol) S1, 13.5 g (50 mmol) 2-amino-1,1′-dinaphthalene, 0.9 g (1 mmol) tri(dibenzylidene acetone) dipalladium, 500 mL toluene were added to a 1000 mL single-necked flask, and vacuumized for nitrogen exchange for 3 times, and 0.5 mL tri-tert-butylphosphine toluene solution was added, and heated up to 110° C. for reaction for 12 h; at the end of the reaction, solvent was removed by evaporation, and silica-gel column chromatography was performed to obtain N176; M/Z theoretical value: 653, M/Z measured value: 654.

### Synthesis Example 2-13: Synthesis of the Compound N191

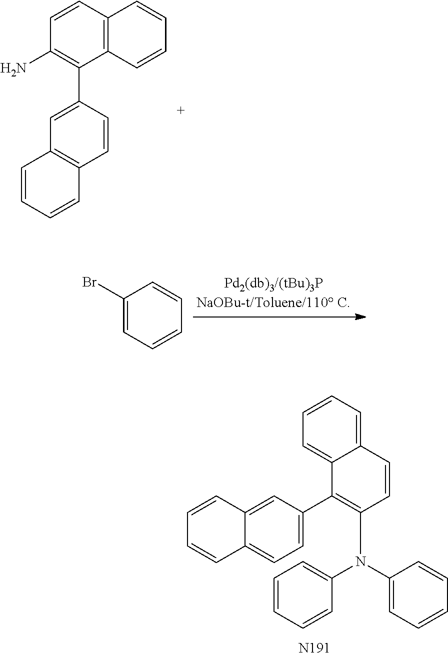 [SMILES: Nc1ccc2ccccc2c1-c1ccc2ccccc2c1 ; Brc1ccccc1 ; c1ccc(N(c2ccccc2)c2ccc3ccccc3c2-c2ccc3ccccc3c2)cc1 ; "N191"]

13.5 g (50 mmol) 2-amino-1,2′-dinaphthalene, 15.7 g (100 mmol) bromobenzene, 0.9 g (1 mmol) tri(dibenzylidene acetone) dipalladium, 0.5 mL tri-tert-butylphosphine, 500 mL toluene, and 14.4 g (150 mmol) sodium tert-butoxide were added to a 1000 mL single-necked flask, and vacuumized for nitrogen exchange for 3 times, then the reaction was heated up to 110° C. for reacting for 5 h. The reaction was terminated at the end of the reaction. The flask was cooled to room temperature, and reaction liquid was separated, and organic phases were concentrated, then methanol was added and stirred for 1 h, and suction filtration was performed to obtain a faint yellow powder N191; M/Z theoretical value: 421, and M/Z measured value: 422.

### Synthesis Example 2-14: Synthesis of the Compound N314

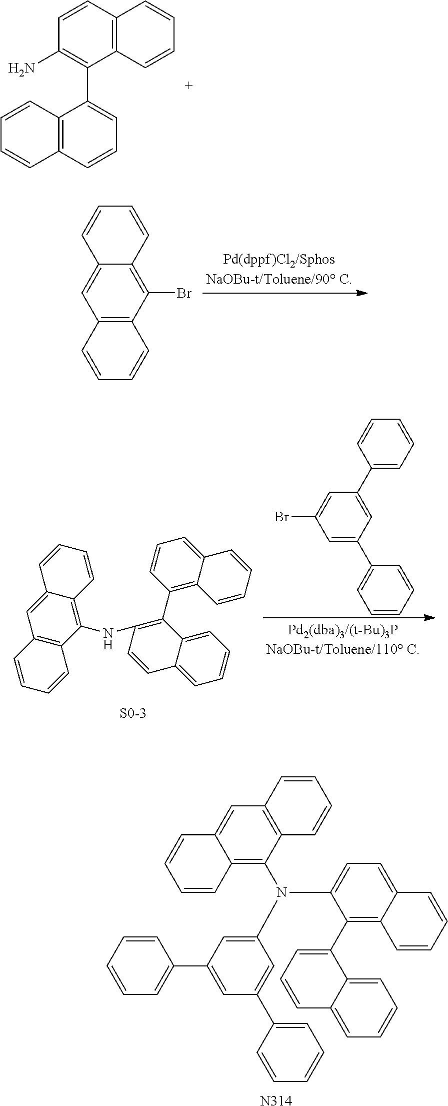

13.5 g (50 mmol) 2-amino-1,1′-dinaphthalene, 13 g (50 mmol) 9-bromoanthracene, 0.7 g (1 mmol) [1,1′-bis(diphenylphosphine)ferrocene] palladium dichloride (Pd(dppf)Cl₂), 0.5 g 2-bicyclohexylphosphine-2′,6′-dimethoxybiphenyl, 500 mL toluene, and 14.4 g (150 mmol) sodium tert-butoxide were added to a 1000 mL single-necked flask, and vacuumized for nitrogen exchange for 3 times, then the reaction was heated up to 90° C. for reacting for 5 h. The reaction was terminated at the end of the reaction. The flask was cooled to room temperature, and reaction liquid was separated, and organic phases were concentrated, then methanol was added and stirred for 1 h, and suction filtration was performed to obtain a faint yellow powder S0-3.

22 g (50 mmol) S0-3, 15 g (50 mmol) 3,5-diphenylbromobenzene, 0.9 g (1 mmol) tri(dibenzylidene acetone) dipalladium, 500 mL toluene were added to a 1000 mL single-necked flask, and vacuumized for nitrogen exchange for 3 times, and 0.5 mL tri-tert-butylphosphine (P(t-Bu)₃) toluene solution was added, and heated up to 110° C. for reaction for 12 h; at the end of the reaction, solvent was removed by evaporation, and silica-gel column chromatography was performed to obtain N314; M/Z theoretical value: 673, M/Z measured value: 674.

### Synthesis Example 2-15: Synthesis of the Compound N325

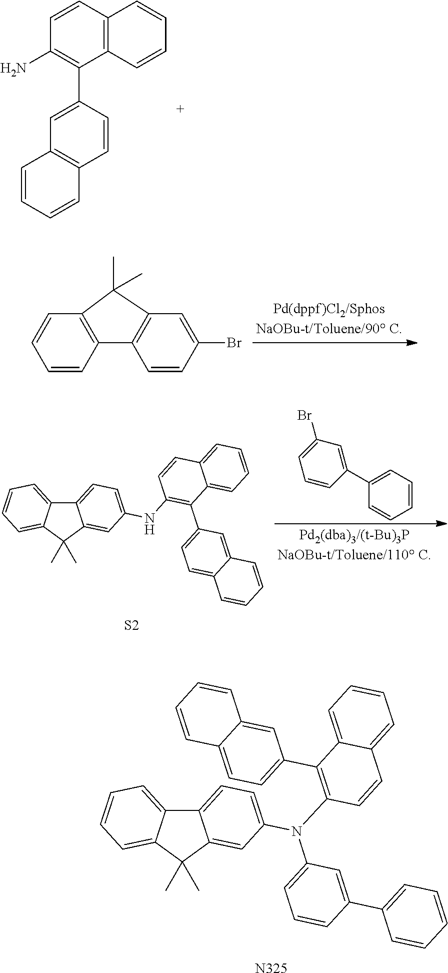

13.5 g (50 mmol) 2-amino-1,2′-dinaphthalene, 13.5 g (50 mmol) 2-bromo-9,9′-dimethylfluorene, 0.7 g (1 mmol) [1,1′-bis(diphenylphosphine)ferrocene] palladium dichloride, 0.5 g 2-bicyclohexylphosphine-2′,6′-dimethoxybiphenyl, 500 mL toluene, and 14.4 g (150 mmol) sodium tert-butoxide were added to a 1000 mL single-necked flask, and vacuumized for nitrogen exchange for 3 times, then the reaction was heated up to 90° C. for reacting for 5 h. The reaction was terminated at the end of the reaction. The flask was cooled to room temperature, and reaction liquid was separated, and organic phases were concentrated, then methanol was added and stirred for 1 h, and suction filtration was performed to obtain a faint yellow powder S2.

23 g (50 mmol) S1, 11.5 g (50 mmol)3-bromo-biphenyl, 0.9 g (1 mmol) tri(dibenzylidene acetone) dipalladium, 500 mL toluene were added to a 1000 mL single-necked flask, and vacuumized for nitrogen exchange for 3 times, and 0.5 mL tri-tert-butylphosphine toluene solution was added, and heated up to 110° C. for reaction for 12 h; at the end of the reaction, solvent was removed by evaporation, and silica-gel column chromatography was performed to obtain N325; M/Z theoretical value: 613, M/Z measured value: 614.

### Synthesis Example 2-16: Synthesis of the Compound N331

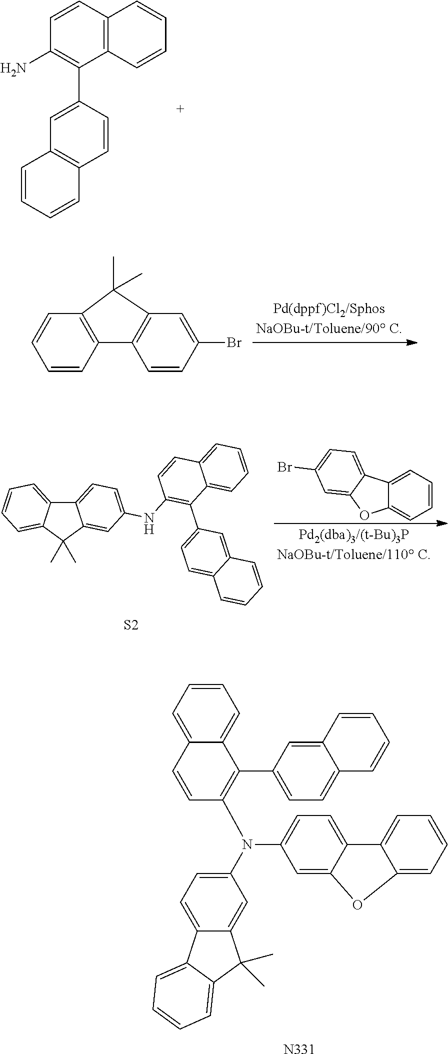

13.5 g (50 mmol) 2-amino-1,2′-dinaphthalene, 13.5 g (50 mmol) 2-bromo-9,9′-dimethylfluorene, 0.7 g (1 mmol) [1,1′-bis(diphenylphosphine)ferrocene] palladium dichloride, 0.5 g 2-bicyclohexylphosphine-2′,6′-dimethoxybiphenyl, 500 mL toluene, and 14.4 g (150 mmol) sodium tert-butoxide were added to a 1000 mL single-necked flask, and vacuumized for nitrogen exchange for 3 times, then the reaction was heated up to 90° C. for reacting for 5 h. The reaction was terminated at the end of the reaction. The flask was cooled to room temperature, and reaction liquid was separated, and organic phases were concentrated, then methanol was added and stirred for 1 h, and suction filtration was performed to obtain a faint yellow powder S2.

23 g (50 mmol) S2, 12.3 g (50 mmol) 2-bromo-dibenzofuran, 0.9 g (1 mmol) tri(dibenzylidene acetone) dipalladium, 500 mL toluene were added to a 1000 mL single-necked flask, and vacuumized for nitrogen exchange for 3 times, and 0.5 mL tri-tert-butylphosphine toluene solution was added, and heated up to 110° C. for reaction for 12 h; at the end of the reaction, solvent was removed by evaporation, and silica-gel column chromatography was performed to obtain N331; M/Z theoretical value: 627, M/Z measured value: 628.

### Synthesis Example 2-17: Synthesis of the Compound N337

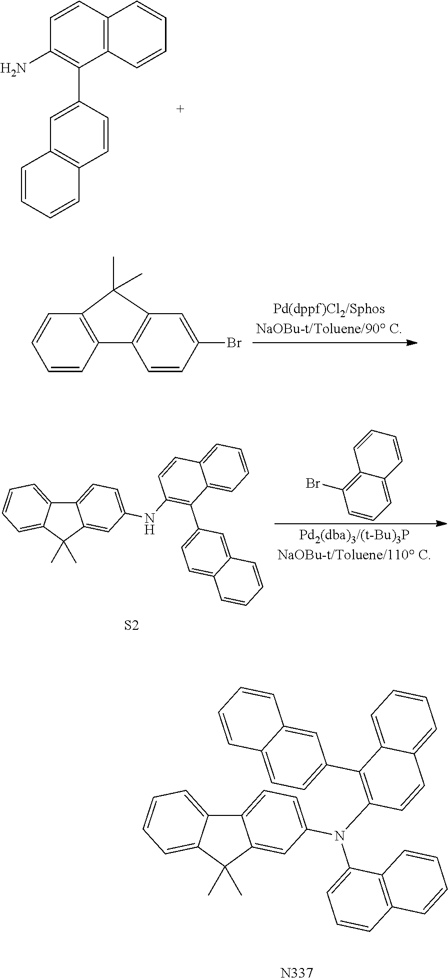

13.5 g (50 mmol) 2-amino-1,2′-dinaphthalene, 13.5 g (50 mmol) 2-bromo-9,9′-dimethylfluorene, 0.7 g (1 mmol) [1,1′-bis(diphenylphosphine)ferrocene] palladium dichloride, 0.5 g 2-bicyclohexylphosphine-2,6′-dimethoxybiphenyl, 500 mL toluene, and 14.4 g (150 mmol) sodium tert-butoxide were added to a 1000 mL single-necked flask, and vacuumized for nitrogen exchange for 3 times, then the reaction was heated up to 90° C. for reacting for 5 h. The reaction was terminated at the end of the reaction. The flask was cooled to room temperature, and reaction liquid was separated, and organic phases were concentrated, then methanol was added and stirred for 1 h, and suction filtration was performed to obtain a faint yellow powder S2.

23 g (50 mmol) S2, 10.4 g (50 mmol) 2-bromonaphthalene, 0.9 g (1 mmol) tri(dibenzylidene acetone) dipalladium, 500 mL toluene were added to a 1000 mL single-necked flask, and vacuumized for nitrogen exchange for 3 times, and 0.5 mL tri-tert-butylphosphine toluene solution was added, and heated up to 110° C. for reaction for 12 h; at the end of the reaction, solvent was removed by evaporation, and silica-gel column chromatography was performed to obtain N337; M/Z theoretical value: 587, M/Z measured value: 588.

### Synthesis Example 2-18: Synthesis of the Compound N371

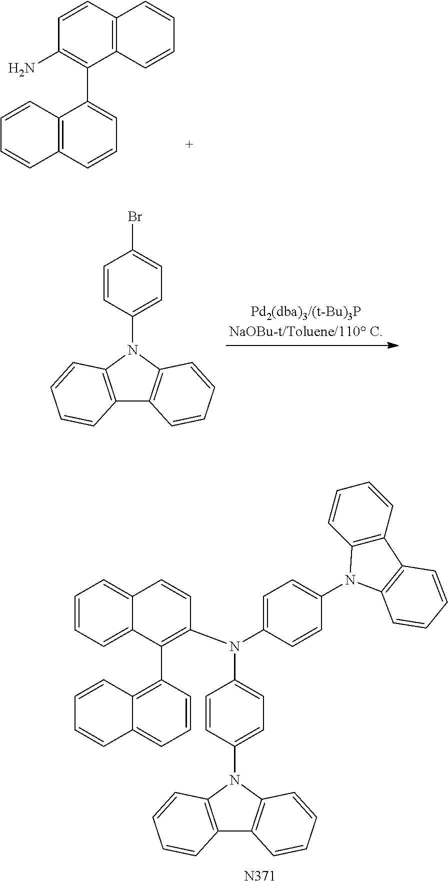 [SMILES: Nc1ccc2ccccc2c1-c1cccc2ccccc12 ; Brc1ccc(-n2c3ccccc3c3ccccc32)cc1 ; c1ccc2c(-c3c(N(c4ccc(-n5c6ccccc6c6ccccc65)cc4)c4ccc(-n5c6ccccc6c6ccccc65)cc4)ccc4ccccc34)cccc2c1 ; "N371"]

13.5 g (50 mmol) 2-amino-1,1′-dinaphthalene, 32.2 g (100 mmol) 9-(4-bromophenyl)-carbazole, 0.9 g (1 mmol) tri(dibenzylidene acetone) dipalladium, 0.5 mL tri-tert-butylphosphine, 500 mL toluene, and 14.4 g (150 mmol) sodium tert-butoxide were added to a 1000 mL single-necked flask, and vacuumized for nitrogen exchange for 3 times, then the reaction was heated up to 110° C. for reacting for 5 h. The reaction was terminated at the end of the reaction. The flask was cooled to room temperature, and reaction liquid was separated, and organic phases were concentrated, then methanol was added and stirred for 1 h, and suction filtration was performed to obtain a faint yellow powder N371; M/Z theoretical value: 751, and M/Z measured value: 752.

### Synthesis Example 2-19: Synthesis of the Compound N372

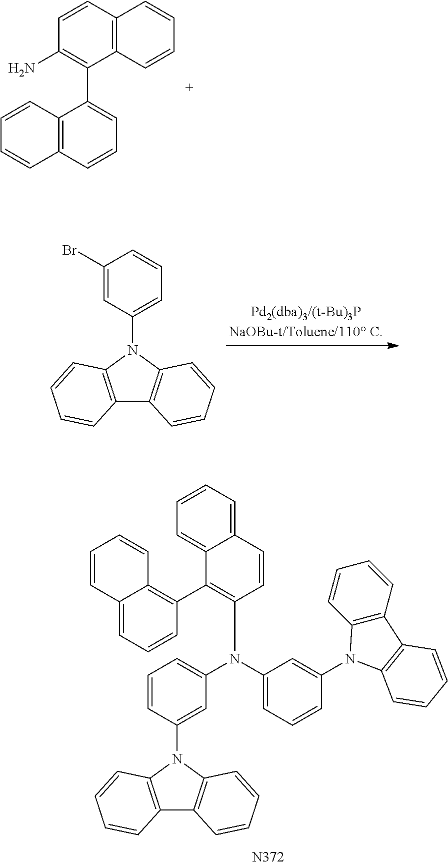

13.5 g (50 mmol) 2-amino-1,1′-dinaphthalene, 32.2 g (100 mmol) 9-(3-bromophenyl-carbazole, 0.9 g (1 mmol) tri(dibenzylidene acetone) dipalladium, 0.5 mL tri-tert-butylphosphine, 500 mL toluene, and 14.4 g (150 mmol) sodium tert-butoxide were added to a 1000 mL single-necked flask, and vacuumized for nitrogen exchange for 3 times, then the reaction was heated up to 110° C. for reacting for 5 h. The reaction was terminated at the end of the reaction. The flask was cooled to room temperature, and reaction liquid was separated, and organic phases were concentrated, then methanol was added and stirred for 1 h, and suction filtration was performed to obtain a faint yellow powder N372; M/Z theoretical value: 751, and M/Z measured value: 752.

### Synthesis Example 2-20: Synthesis of the Compound N373

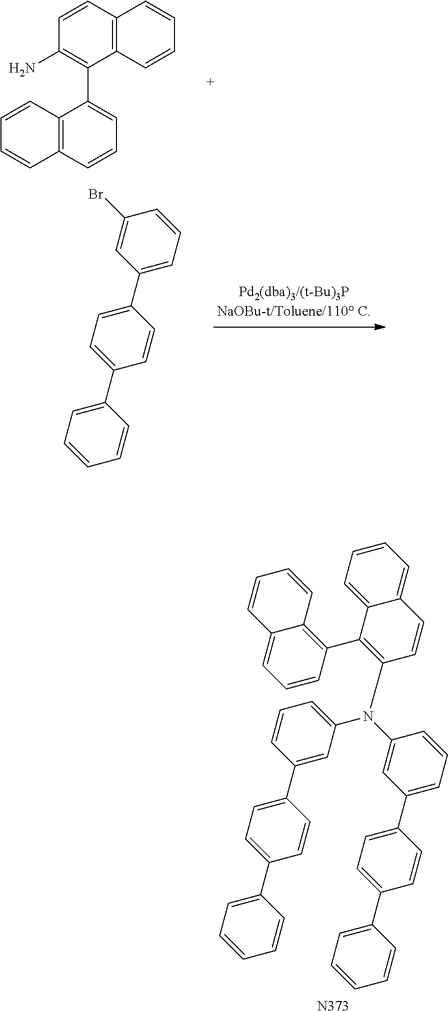

13.5 g (50 mmol) 2-amino-1,1′-dinaphthalene, 30.9 g (100 mmol) 3-bromoterphenyl, 0.9 g (1 mmol) tri(dibenzylidene acetone) dipalladium, 0.5 mL tri-tert-butylphosphine, 500 mL toluene, and 14.4 g (150 mmol) sodium tert-butoxide were added to a 1000 mL single-necked flask, and vacuumized for nitrogen exchange for 3 times, then the reaction was heated up to 110° C. for reacting for 5 h. The reaction was terminated at the end of the reaction. The flask was cooled to room temperature, and reaction liquid was separated, and organic phases were concentrated, then methanol was added and stirred for 1 h, and suction filtration was performed to obtain a faint yellow powder N373; M/Z theoretical value: 725, and M/Z measured value: 726.

### Synthesis Example 2-21: Synthesis of the Compound N374

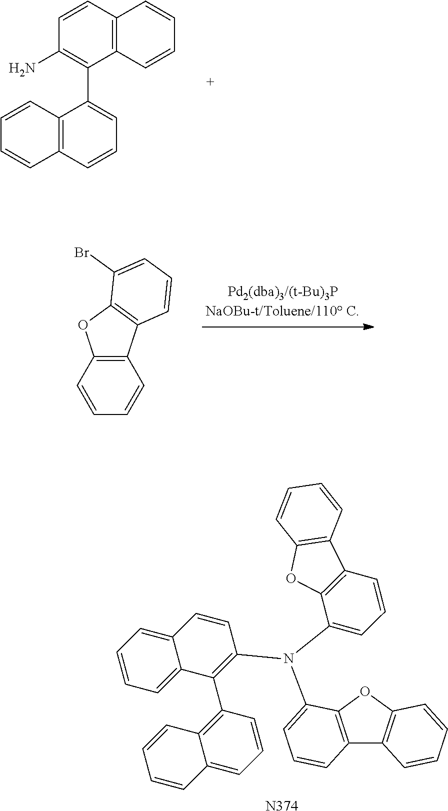

13.5 g (50 mmol) 2-amino-1,1′-dinaphthalene, 24.5 g (100 mmol) 4-bromodibenzofuran, 0.9 g (1 mmol) tri(dibenzylidene acetone) dipalladium, 0.5 mL tri-tert-butylphosphine, 500 mL toluene, and 14.4 g (150 mmol) sodium tert-butoxide were added to a 1000 mL single-necked flask, and vacuumized for nitrogen exchange for 3 times, then the reaction was heated up to 110° C. for reacting for 5 h. The reaction was terminated at the end of the reaction. The flask was cooled to room temperature, and reaction liquid was separated, and organic phases were concentrated, then methanol was added and stirred for 1 h, and suction filtration was performed to obtain a faint yellow powder N374; M/Z theoretical value: 601.31, and M/Z measured value: 602.

### Synthesis Example 2-22: Synthesis of the Compound N375

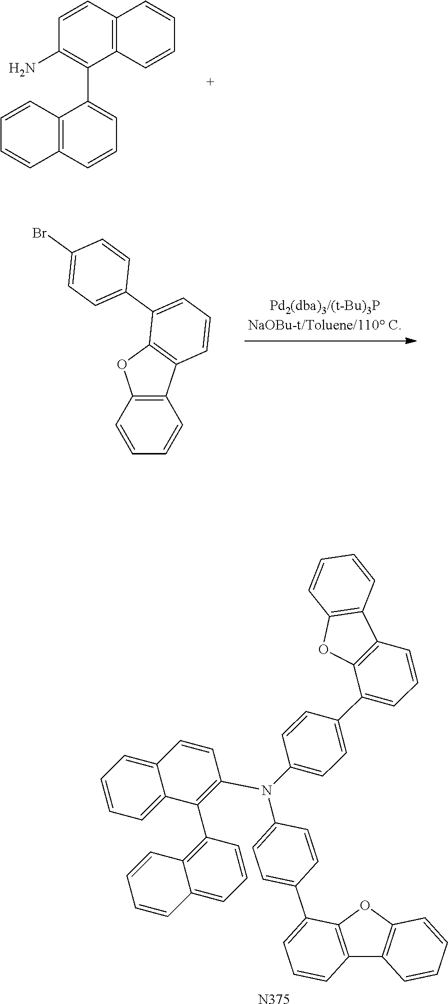 [SMILES: Nc1ccc2ccccc2c1-c1cccc2ccccc12 ; Brc1ccc(-c2cccc3c2oc2ccccc23)cc1 ; c1ccc2c(-c3c(N(c4ccc(-c5cccc6c5oc5ccccc56)cc4)c4ccc(-c5cccc6c5oc5ccccc56)cc4)ccc4ccccc34)cccc2c1 ; "N375"]

13.5 g (50 mmol) 2-amino-1,1′-dinaphthalene, 32.3 g (100 mmol) 4-(4-bromophenyl)-dibenzofuran, 0.9 g (1 mmol) tri(dibenzylidene acetone) dipalladium, 0.5 mL tri-tert-butylphosphine, 500 mL toluene, and 14.4 g (150 mmol) sodium tert-butoxide were added to a 1000 mL single-necked flask, and vacuumized for nitrogen exchange for 3 times, then the reaction was heated up to 110° C. for reacting for 5 h. The reaction was terminated at the end of the reaction. The flask was cooled to room temperature, and reaction liquid was separated, and organic phases were concentrated, then methanol was added and stirred for 1 h, and suction filtration was performed to obtain a faint yellow powder N375; M/Z theoretical value: 753, and M/Z measured value: 754.

### Synthesis Example 2-23: Synthesis of the Compound N376

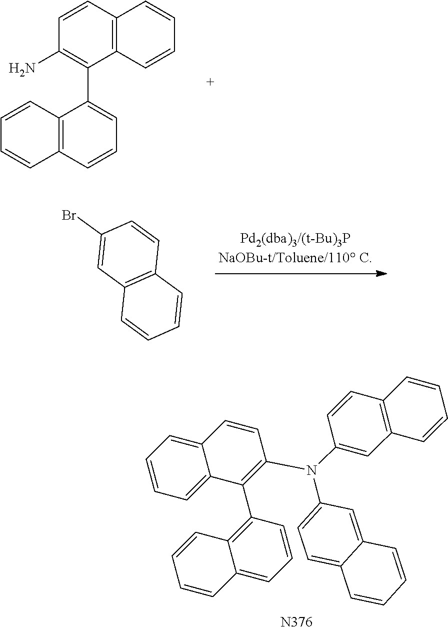

13.5 g (50 mmol) 2-amino 0.5 mL-1,1′-dinaphthalene, 10 g (100 mmol) 2-bromonaphthalene, 0.9 g (1 mmol) tri(dibenzylidene acetone) dipalladium, tri-tert-butylphosphine, 500 mL toluene, and 14.4 g (150 mmol) sodium tert-butoxide were added to a 1000 mL single-necked flask, and vacuumized for nitrogen exchange for 3 times, then the reaction was heated up to 110° C. for reacting for 5 h. The reaction was terminated at the end of the reaction. The flask was cooled to room temperature, and reaction liquid was separated, and organic phases were concentrated, then methanol was added and stirred for 1 h, and suction filtration was performed to obtain a faint yellow powder N376; M/Z theoretical value: 521, and M/Z measured value: 522.

### Synthesis Example 2-24: Synthesis of the Compound N377

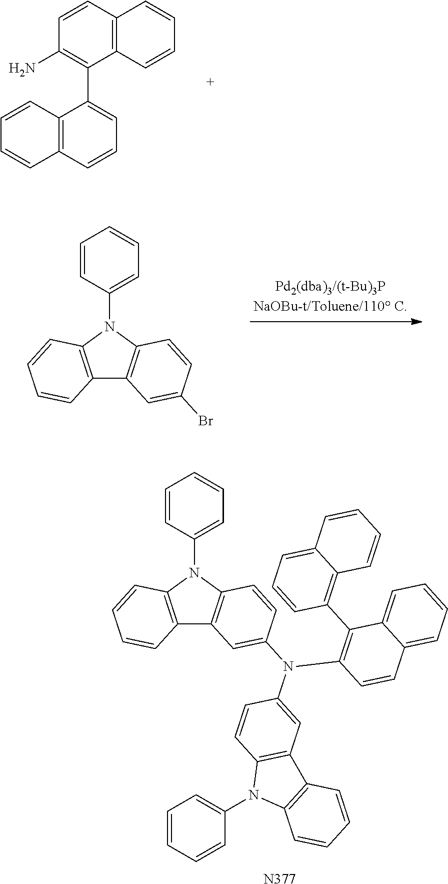 [SMILES: Nc1ccc2ccccc2c1-c1cccc2ccccc12 ; Brc1ccc2c(c1)c1ccccc1n2-c1ccccc1 ; c1ccc(-n2c3ccccc3c3cc(N(c4ccc5c(c4)c4ccccc4n5-c4ccccc4)c4ccc5ccccc5c4-c4cccc5ccccc45)ccc32)cc1 ; "N377"]

13.5 g (50 mmol) 2-amino-1,1′-dinaphthalene, 32.2 g (100 mmol) (9-phenyl)-3-bromocarbazole, 0.9 g (1 mmol) tri(dibenzylidene acetone) dipalladium, 0.5 mL tri-tert-butylphosphine, 500 mL toluene, and 14.4 g (150 mmol) sodium tert-butoxide were added to a 1000 mL single-necked flask, and vacuumized for nitrogen exchange for 3 times, then the reaction was heated up to 110° C. for reacting for 5 h. The reaction was terminated at the end of the reaction. The flask was cooled to room temperature, and reaction liquid was separated, and organic phases were concentrated, then methanol was added and stirred for 1 h, and suction filtration was performed to obtain a faint yellow powder N377; M/Z theoretical value: 751, and M/Z measured value: 752.

### Synthesis Example 2-25: Synthesis of the Compound N378

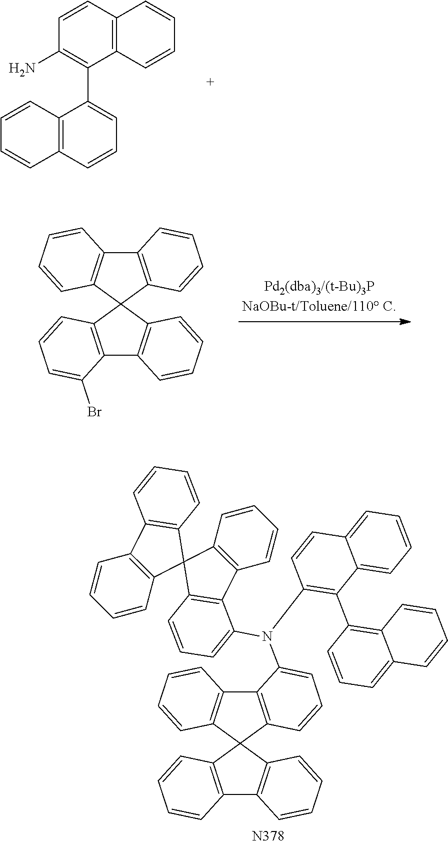

6.7 g (25 mmol) 2-amino-1,1′-dinaphthalene, 20 g (100 mmol) 4-bromo-9,9′-spirobifluorene, 0.9 g (1 mmol) tri(dibenzylidene acetone) dipalladium, 0.5 mL tri-tert-butylphosphine, 500 mL toluene, and 14.4 g (150 mmol) sodium tert-butoxide were added to a 1000 mL single-necked flask, and vacuumized for nitrogen exchange for 3 times, then the reaction was heated up to 110° C. for reacting for 5 h. The reaction was terminated at the end of the reaction. The flask was cooled to room temperature, and reaction liquid was separated, and organic phases were concentrated, then methanol was added and stirred for 1 h, and suction filtration was performed to obtain a faint yellow powder N378; M/Z theoretical value: 898, and M/Z measured value: 898.

### Synthesis Example 2-26: Synthesis of the Compound N379

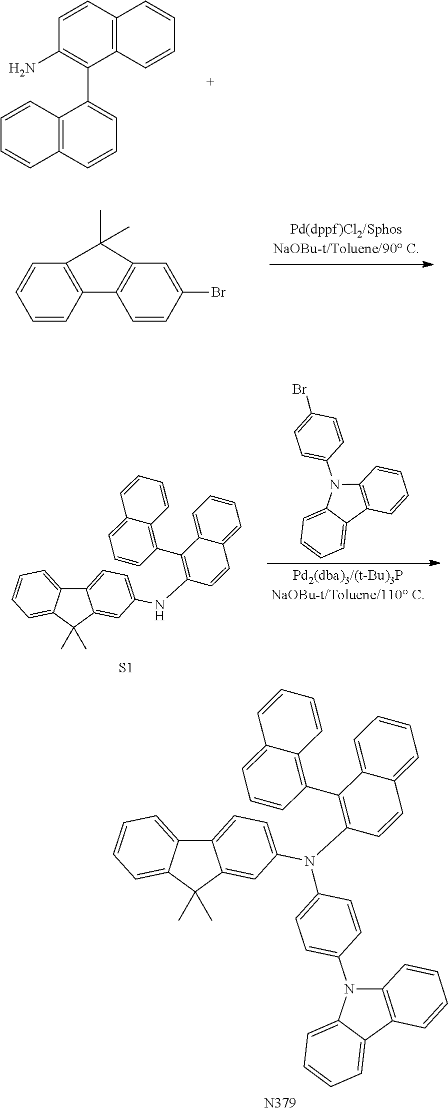

13.5 g (50 mmol) 2-amino-1,1′-dinaphthalene, 13.5 g (50 mmol) 2-bromo-9,9′-dimethylfluorene, 0.7 g (1 mmol) [1,1′-bis(diphenylphosphine)ferrocene] palladium dichloride, 0.5 g 2-bicyclohexylphosphine-2′,6′-dimethoxybiphenyl, 500 mL toluene, and 14.4 g (150 mmol) sodium tert-butoxide were added to a 1000 mL single-necked flask, and vacuumized for nitrogen exchange for 3 times, then the reaction was heated up to 90° C. for reacting for 5 h. The reaction was terminated at the end of the reaction. The flask was cooled to room temperature, and reaction liquid was separated, and organic phases were concentrated, then methanol was added and stirred for 1 h, and suction filtration was performed to obtain a faint yellow powder S1.

23 g (50 mmol) S1, 32.2 g (100 mmol) 9-(4-bromophenyl)-carbazole, 0.9 g (1 mmol) tri(dibenzylidene acetone) dipalladium, 500 mL toluene were added to a 1000 mL single-necked flask, and vacuumized for nitrogen exchange for 3 times, and 0.5 mL tri-tert-butylphosphine toluene solution was added, and heated up to 110° C. for reaction for 12 h; at the end of the reaction, solvent was removed by evaporation, and silica-gel column chromatography was performed to obtain N379; M/Z theoretical value: 702, M/Z measured value: 703.

### Synthesis Example 2-27: Synthesis of the Compound N380

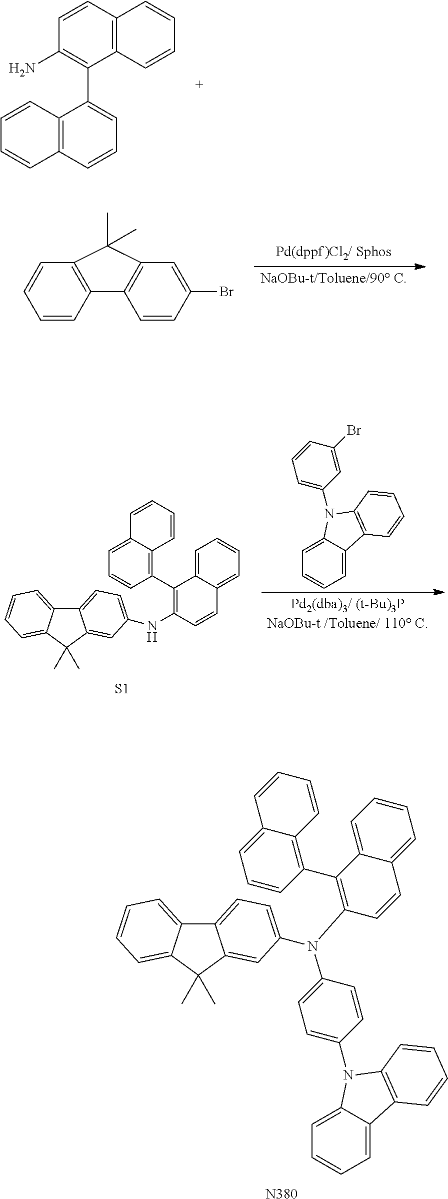

13.5 g (50 mmol) 2-amino-1,1′-dinaphthalene, 13.5 g (50 mmol) 2-bromo-9,9′-dimethylfluorene, 0.7 g (1 mmol) [1,1′-bis(diphenylphosphine)ferrocene] palladium dichloride, 0.5 g 2-bicyclohexylphosphine-2,6′-dimethoxybiphenyl, 500 mL toluene, and 14.4 g (150 mmol) sodium tert-butoxide were added to a 1000 mL single-necked flask, and vacuumized for nitrogen exchange for 3 times, then the reaction was heated up to 90° C. for reacting for 5 h. The reaction was terminated at the end of the reaction. The flask was cooled to room temperature, and reaction liquid was separated, and organic phases were concentrated, then methanol was added and stirred for 1 h, and suction filtration was performed to obtain a faint yellow powder S1.

23 g (50 mmol) S1, 32.2 g (100 mmol) 9-(3-bromophenyl)-carbazole, 0.9 g (1 mmol) tri(dibenzylidene acetone) dipalladium, 500 mL toluene were added to a 1000 mL single-necked flask, and vacuumized for nitrogen exchange for 3 times, and 0.5 mL tri-tert-butylphosphine toluene solution was added, and heated up to 110° C. for reaction for 12 h; at the end of the reaction, the reaction was terminated. Solvent was removed by evaporation, and silica-gel column chromatography was performed to obtain N380; M/Z theoretical value: 702, M/Z measured value: 703.

### Synthesis Example 2-28: Synthesis of the Compound N381

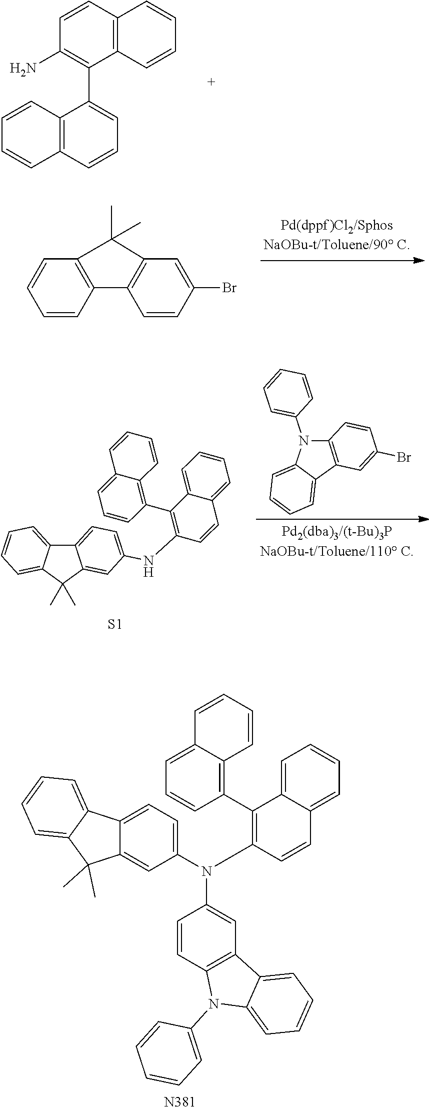

13.5 g (50 mmol) 2-amino-1,1′-dinaphthalene, 13.5 g (50 mmol) 2-bromo-9,9′-dimethylfluorene, 0.7 g (1 mmol) [1,1′-bis(diphenylphosphine)ferrocene] palladium dichloride, 0.5 g 2-bicyclohexylphosphine-2′,6′-dimethoxybiphenyl, 500 mL toluene, and 14.4 g (150 mmol) sodium tert-butoxide were added to a 1000 mL single-necked flask, and vacuumized for nitrogen exchange for 3 times, then the reaction was heated up to 90° C. for reacting for 5 h. The reaction was terminated at the end of the reaction. The flask was cooled to room temperature, and reaction liquid was separated, and organic phases were concentrated, then methanol was added and stirred for 1 h, and suction filtration was performed to obtain a faint yellow powder S1.

23 g (50 mmol) S1, 16.1 g (100 mmol) (9-phenyl)-3-bromocarbazole, 0.9 g (1 mmol) tri(dibenzylidene acetone) dipalladium, 500 mL toluene were added to a 1000 mL single-necked flask, and vacuumized for nitrogen exchange for 3 times, and 0.5 mL tri-tert-butylphosphine toluene solution was added, and heated up to 110° C. for reaction for 12 h; at the end of the reaction, solvent was removed by evaporation, and silica-gel column chromatography was performed to obtain N381; M/Z theoretical value: 702, M/Z measured value: 703.

### Synthesis Example 2-29: Synthesis of the Compound N382

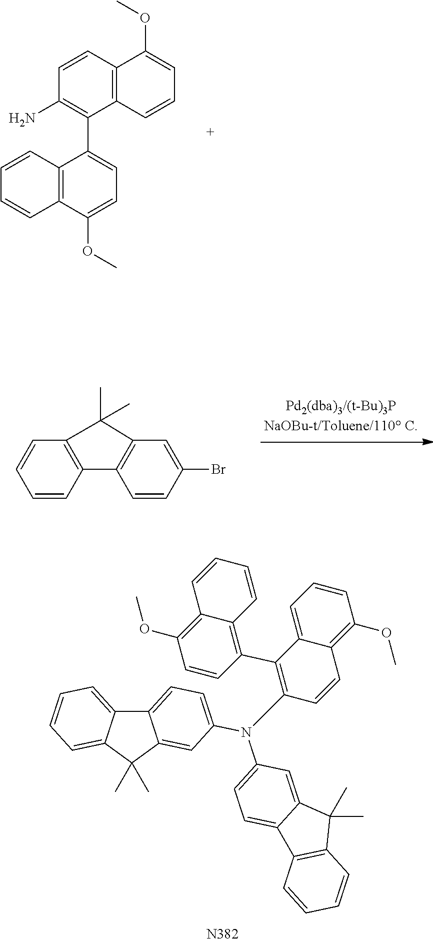 [SMILES: COc1ccc(-c2c(N)ccc3c(OC)cccc23)c2ccccc12 ; CC1(C)c2ccccc2-c2ccc(Br)cc21 ; COc1ccc(-c2c(N(c3ccc4c(c3)C(C)(C)c3ccccc3-4)c3ccc4c(c3)C(C)(C)c3ccccc3-4)ccc3c(OC)cccc23)c2ccccc12 ; "N382"]

13.5 g (50 mmol) 2-amino-4-methoxy-5′-methoxy-1,1′-dinaphthalene, 27 g (100 mmol) 2-bromo-9,9′-dimethylfluorene, 0.9 g (1 mmol) tri(dibenzylidene acetone) dipalladium, 0.5 mL tri-tert-butylphosphine, 500 mL toluene, and 14.4 g (150 mmol) sodium tert-butoxide were added to a 1000 mL single-necked flask, and vacuumized for nitrogen exchange for 3 times, then the reaction was heated up to 110° C. for reacting for 5 h. The reaction was terminated at the end of the reaction. The flask was cooled to room temperature, and reaction liquid was separated, and organic phases were concentrated, then methanol was added and stirred for 1 h, and suction filtration was performed to obtain a faint yellow powder N382; M/Z theoretical value: 713, and M/Z measured value: 714.

### Synthesis Example 2-30: Synthesis of the Compound N383

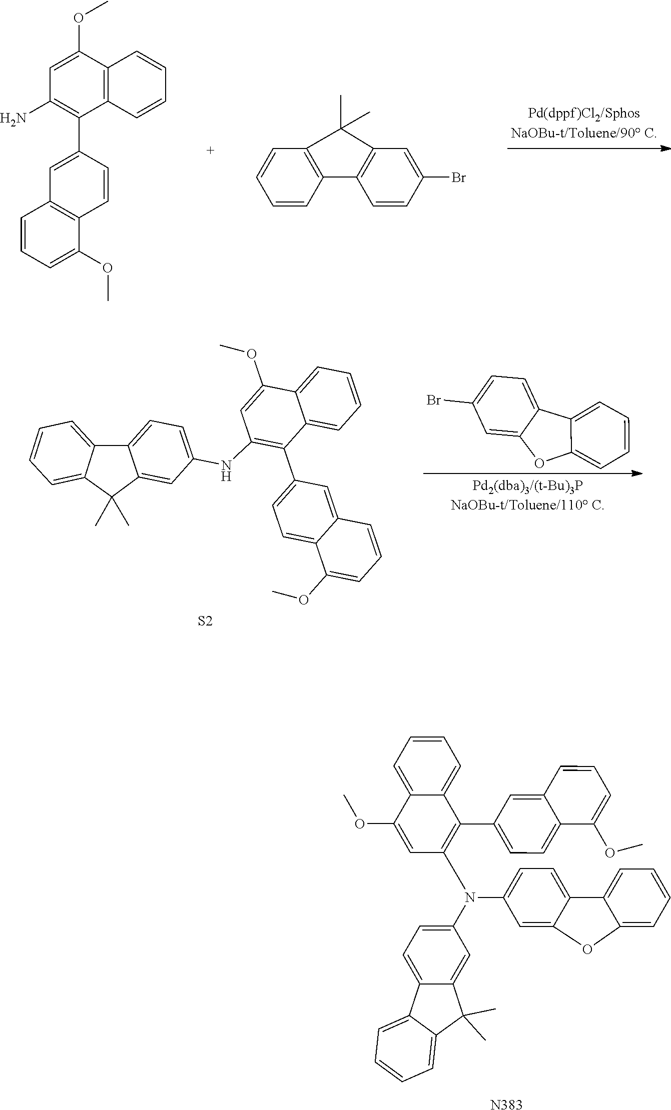

13.5 g (50 mmol) 2-amino-4-methoxy-5′-methoxy-1,2′-dinaphthalene, 13.5 g (50 mmol) 2-bromo-9,9′-dimethylfluorene, 0.7 g (1 mmol) [1,1′-bis(diphenylphosphine)ferrocene] palladium dichloride, 0.5 g 2-bicyclohexylphosphine-2′,6′-dimethoxybiphenyl, 500 mL toluene, and 14.4 g (150 mmol) sodium tert-butoxide were added to a 1000 mL single-necked flask, and vacuumized for nitrogen exchange for 3 times, then the reaction was heated up to 90° C. for reacting for 5 h. The reaction was terminated at the end of the reaction. The flask was cooled to room temperature, and reaction liquid was separated, and organic phases were concentrated, then methanol was added and stirred for 1 h, and suction filtration was performed to obtain a faint yellow powder S2.

23 g (50 mmol) S2, 12.3 g (50 mmol) 2-bromo-dibenzofuran, 0.9 g (1 mmol) tri(dibenzylidene acetone) dipalladium, 500 mL toluene were added to a 1000 mL single-necked flask, and vacuumized for nitrogen exchange for 3 times, and 0.5 mL tri-tert-butylphosphine toluene solution was added, and heated up to 110° C. for reaction for 12 h; at the end of the reaction, the reaction was terminated. Solvent was removed by evaporation, and silica-gel column chromatography was performed to obtain N383; M/Z theoretical value: 687, M/Z measured value: 688.

### Synthesis Example 2-31: Synthesis of the Compound N387

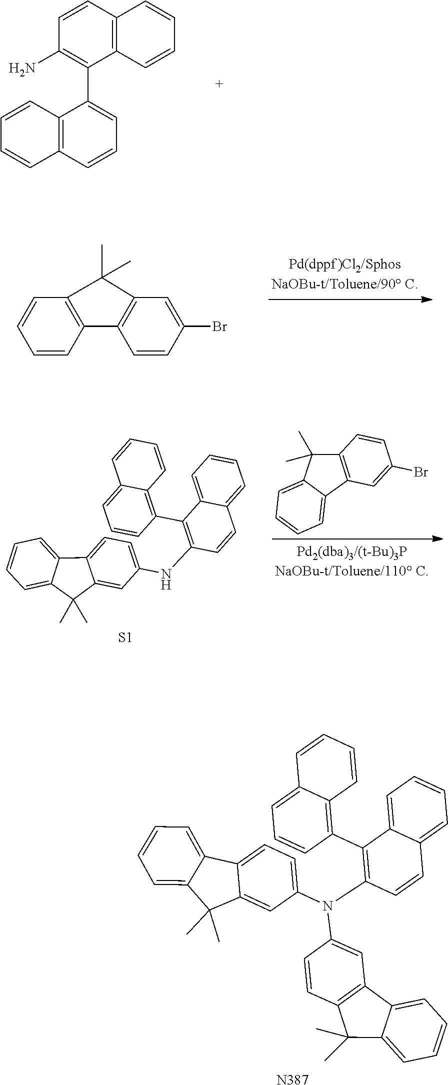

13.5 g (50 mmol) 2-amino-1,1′-dinaphthalene, 13.5 g (50 mmol) 2-bromo-9,9′-dimethylfluorene, 0.7 g (1 mmol) [1,1′-bis(diphenylphosphine)ferrocene] palladium dichloride, 0.5 g 2-bicyclohexylphosphine-2′,6′-dimethoxybiphenyl, 500 mL toluene, and 14.4 g (150 mmol) sodium tert-butoxide were added to a 1000 mL single-necked flask, and vacuumized for nitrogen exchange for 3 times, then the reaction was heated up to 90° C. for reacting for 5 h. The reaction was terminated at the end of the reaction. The flask was cooled to room temperature, and reaction liquid was separated, and organic phases were concentrated, then methanol was added and stirred for 1 h, and suction filtration was performed to obtain a faint yellow powder S1.

23 g (50 mmol) S1, 13.5 g (100 mmol) 3-bromo-9,9′-dimethylfluorene, 0.9 g (1 mmol) tri(dibenzylidene acetone) dipalladium, 500 mL toluene were added to a 1000 mL single-necked flask, and vacuumized for nitrogen exchange for 3 times, and 0.5 mL tri-tert-butylphosphine toluene solution was added, and heated up to 110° C. for reaction for 12 h; at the end of the reaction, solvent was removed by evaporation, and silica-gel column chromatography was performed to obtain N387; M/Z theoretical value: 653, M/Z measured value: 654.

### Synthesis Example 2-32: Synthesis of the Compound N389

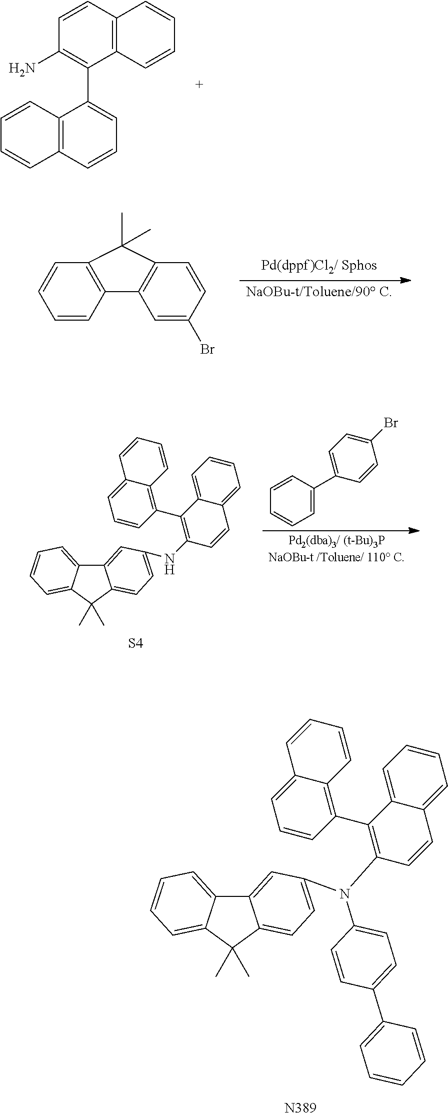

13.5 g (50 mmol) 2-amino-1,1′-dinaphthalene, 13.5 g (50 mmol) 3-bromo-9,9′-dimethylfluorene, 0.7 g (1 mmol) [1,1′-bis(diphenylphosphine)ferrocene] palladium dichloride, 0.5 g 2-bicyclohexylphosphine-2′,6′-dimethoxybiphenyl, 500 mL toluene, and 14.4 g (150 mmol) sodium tert-butoxide were added to a 1000 mL single-necked flask, and vacuumized for nitrogen exchange for 3 times then the reaction was heated up to 90° C. for reacting for 5 h. The reaction was terminated at the end of the reaction. The flask was cooled to room temperature, and reaction liquid was separated, and organic phases were concentrated, then methanol was added and stirred for 1 h, and suction filtration was performed to obtain a faint yellow powder S4.

23 g (50 mmol) S4, 12 g (100 mmol) p-bromo-biphenyl, 0.9 g (1 mmol) tri(dibenzylidene acetone) dipalladium, 500 mL toluene were added to a 1000 mL single-necked flask, and vacuumized for nitrogen exchange for 3 times, and 0.5 mL tri-tert-butylphosphine toluene solution was added, and heated up to 11000 for reaction for 12 h; at the end of the reaction, solvent was removed by evaporation, and silica-gel column chromatography was performed to obtain N389; M/Z theoretical value: 633, M/Z measured value: 634.

### Synthesis Example 2-33: Synthesis of the Compound N396

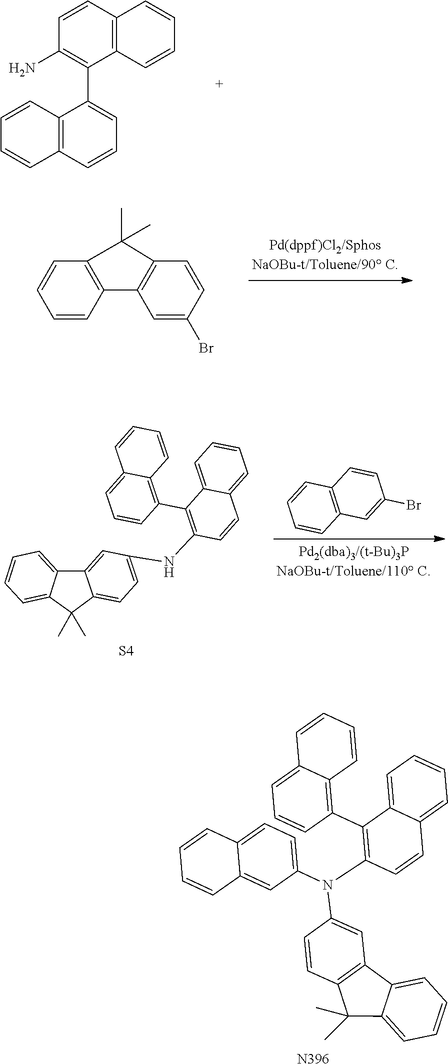

13.5 g (50 mmol) 2-amino-1,1′-dinaphthalene, 13.5 g (50 mmol) 3-bromo-9,9′-dimethylfluorene, 0.7 g (1 mmol) [1,1′-bis(diphenylphosphine)ferrocene] palladium dichloride, 0.5 g 2-bicyclohexylphosphine-2′,6′-dimethoxybiphenyl, 500 mL toluene, and 14.4 g (150 mmol) sodium tert-butoxide were added to a 1000 mL single-necked flask, and vacuumized for nitrogen exchange for 3 times, then the reaction was heated up to 90° C. for reacting for 5 h. The reaction was terminated at the end of the reaction. The flask was cooled to room temperature, and reaction liquid was separated, and organic phases were concentrated, then methanol was added and stirred for 1 h, and suction filtration was performed to obtain a faint yellow powder S4.

23 g (50 mmol) S4, 10.5 g (100 mmol) 2-bromonaphthalene, 0.9 g (1 mmol) tri(dibenzylidene acetone) dipalladium, 500 mL toluene were added to a 1000 mL single-necked flask, and vacuumized for nitrogen exchange for 3 times, and 0.5 mL tri-tert-butylphosphine toluene solution was added, and heated up to 110° C. for reaction for 12 h; at the end of the reaction, solvent was removed by evaporation, and silica-gel column chromatography was performed to obtain N396; M/Z theoretical value: 587, M/Z measured value: 588.

### Synthesis Example 2-34: Synthesis of the Compound N405

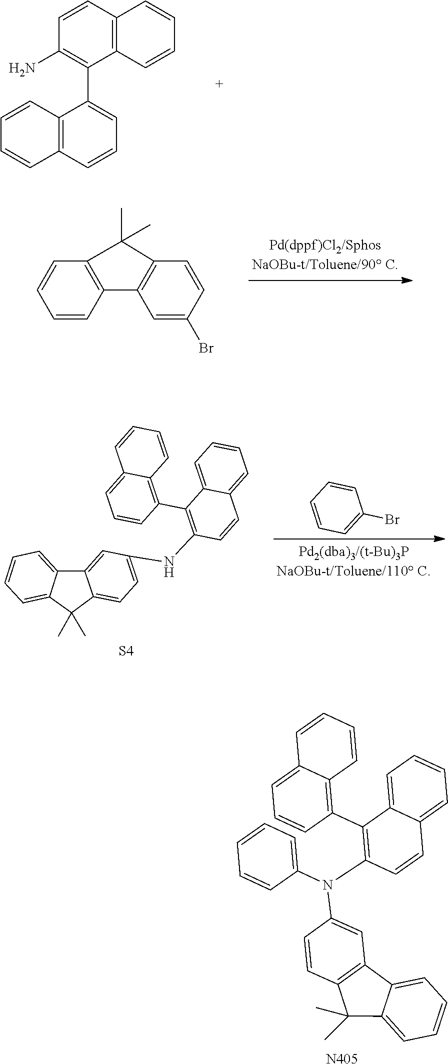

13.5 g (50 mmol) 2-amino-1,1′-dinaphthalene, 13.5 g (50 mmol) 3-bromo-9,9′-dimethyfluorene, 0.7 g (1 mmol) [1,1′-bis(diphenylphosphine)ferrocene] palladium dichloride, 0.5 g 2-bicyclohexylphosphine-2′,6′-dimethoxybiphenyl, 500 mL toluene, and 14.4 g (150 mmol) sodium tert-butoxide were added to a 1000 mL single-necked flask, and vacuumized for nitrogen exchange for 3 times, then the reaction was heated up to 90° C. for reacting for 5 h. The reaction was terminated at the end of the reaction. The flask was cooled to room temperature, and reaction liquid was separated, and organic phases were concentrated, then methanol was added and stirred for 1 h, and suction filtration was performed to obtain a faint yellow powder S4.

23 g (50 mmol) S4, 8.7 g (100 mmol) bromobenzene, 0.9 g (1 mmol) tri(dibenzylidene acetone) dipalladium, 500 mL toluene were added to a 1000 mL single-necked flask, and vacuumized for nitrogen exchange for 3 times, and 0.5 mL tri-tert-butylphosphine toluene solution was added, and heated up to 110° C. for reaction for 12 h; at the end of the reaction, solvent was removed by evaporation, and silica-gel column chromatography was performed to obtain N405; M/Z theoretical value: 537, M/Z measured value: 538.

### Synthesis Example 2-35: Synthesis of the Compound N406

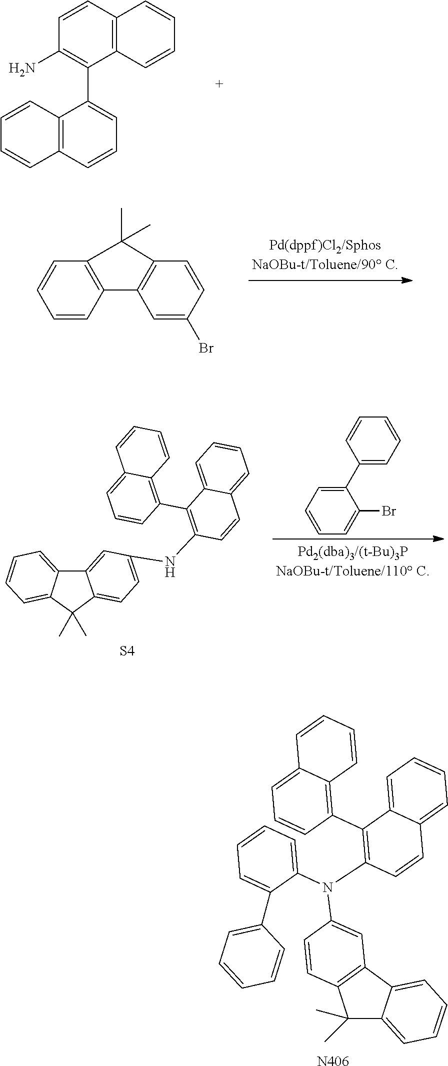

13.5 g (50 mmol) 2-amino-1,1′-dinaphthalene, 13.5 g (50 mmol) 3-bromo-9,9′-dimethyfluorene, 0.7 g (1 mmol) [1,1′-bis(diphenylphosphine)ferrocene] palladium dichloride, 0.5 g 2-bicyclohexylphosphine-2′,6′-dimethoxybiphenyl, 500 mL toluene, and 14.4 g (150 mmol) sodium tert-butoxide were added to a 1000 mL single-necked flask, and vacuumized for nitrogen exchange for 3 times, then the reaction was heated up to 90° C. for reacting for 5 h. The reaction was terminated at the end of the reaction. The flask was cooled to room temperature, and reaction liquid was separated, and organic phases were concentrated, then methanol was added and stirred for 1 h, and suction filtration was performed to obtain a faint yellow powder S4.

23 g (50 mmol) S4, 12 g (100 mmol) 2-bromobiphenyl, 0.9 g (1 mmol) tri(dibenzylidene acetone) dipalladium, 500 mL toluene were added to a 1000 mL single-necked flask, and vacuumized for nitrogen exchange for 3 times, and 0.5 mL tri-tert-butylphosphine toluene solution was added, and heated up to 110° C. for reaction for 12 h; at the end of the reaction, solvent was removed by evaporation, and silica-gel column chromatography was performed to obtain N406; M/Z theoretical value: 613, M/Z measured value: 614.

### Synthesis Example 2-36: Synthesis of the Compound N409

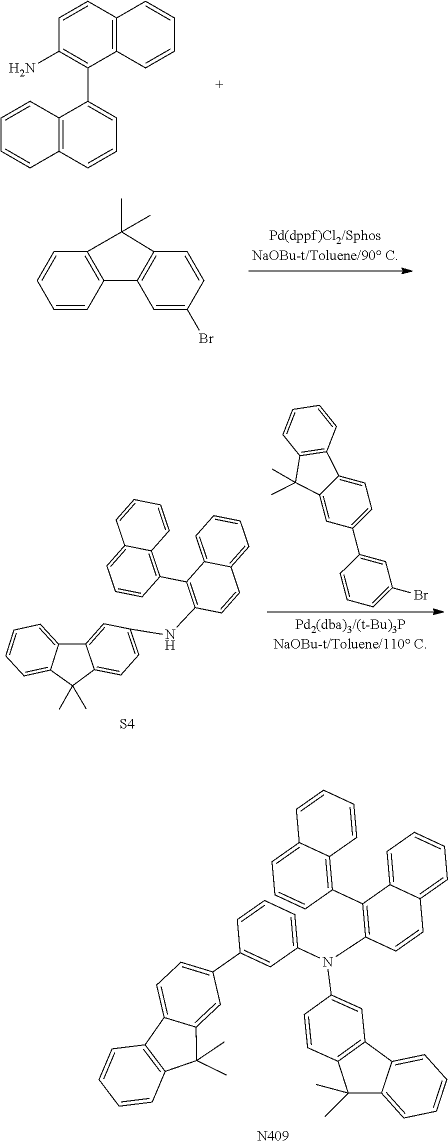

13.5 g (50 mmol) 2-amino-1,1′-dinaphthalene, 13.5 g (50 mmol) 3-bromo-9,9′-dimethylfluorene, 0.7 g (1 mmol) [1,1′-bis(diphenylphosphine)ferrocene] palladium dichloride, 0.5 g 2-bicyclohexylphosphine-2′,6′-dimethoxybiphenyl, 500 mL toluene, and 14.4 g (150 mmol) sodium tert-butoxide were added to a 1000 mL single-necked flask, and vacuumized for nitrogen exchange for 3 times, then the reaction was heated up to 90° C. for reacting for 5 h. The reaction was terminated at the end of the reaction. The flask was cooled to room temperature, and reaction liquid was separated, and organic phases were concentrated, then methanol was added and stirred for 1 h, and suction filtration was performed to obtain a faint yellow powder S4.

23 g (50 mmol) S4, 17.5 g (100 mmol) 3-(2-(9,9-dimethylfluorene)) bromobenzene, 0.9 g (1 mmol) tri(dibenzylidene acetone) dipalladium, 500 mL toluene were added to a 1000 mL single-necked flask, and vacuumized for nitrogen exchange for 3 times, and 0.5 mL tri-tert-butylphosphine toluene solution was added, and heated up to 110° C. for reaction for 12 h; at the end of the reaction, solvent was removed by evaporation, and silica-gel column chromatography was performed to obtain N409; M/Z theoretical value: 729, M/Z measured value: 730.

### Synthesis Example 2-37: Synthesis of the Compound N414

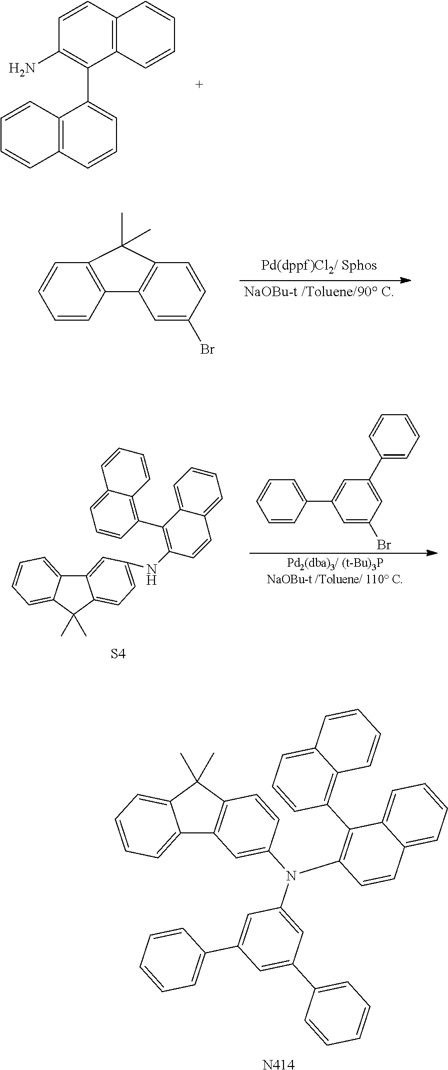

13.5 g (50 mmol) 2-amino-1,1′-dinaphthalene, 13.5 g (50 mmol) 3-bromo-9,9′-dimethylfluorene, 0.7 g (1 mmol) [1,1′-bis(diphenylphosphine)ferrocene] palladium dichloride, 0.5 g 2-bicyclohexylphosphine-2′,6′-dimethoxybiphenyl, 500 mL toluene, and 14.4 g (150 mmol) sodium tert-butoxide were added to a 1000 mL single-necked flask, and vacuumized for nitrogen exchange for 3 times, then the reaction was heated up to 90° C. for reacting for 5 h. The reaction was terminated at the end of the reaction. The flask was cooled to room temperature, and reaction liquid was separated, and organic phases were concentrated, then methanol was added and stirred for 1 h, and suction filtration was performed to obtain a faint yellow powder S4.

23 g (50 mmol) S4, 15 g (100 mmol) 3,5-diphenylbromobenzene, 0.9 g (1 mmol) tri(dibenzylidene acetone) dipalladium, 500 mL toluene were added to a 1000 mL single-necked flask, and vacuumized for nitrogen exchange for 3 times, and 0.5 mL tri-tert-butylphosphine toluene solution was added, and heated up to 110° C. for reaction for 12 h; at the end of the reaction, solvent was removed by evaporation, and silica-gel column chromatography was performed to obtain N414; M/Z theoretical value: 689, M/Z measured value: 690.

### Synthesis Example 2-38: Synthesis of the Compound N418

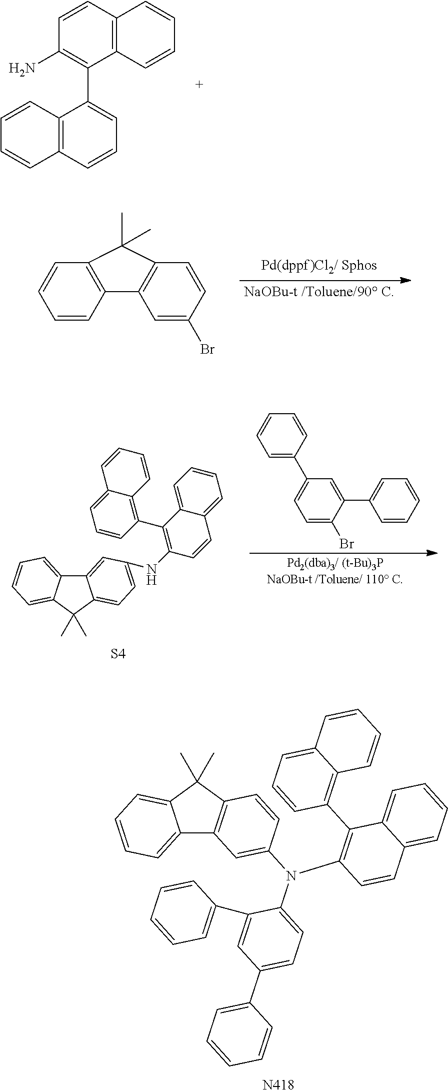

13.5 g (50 mmol) 2-amino-1,1′-dinaphthalene, 13.5 g (50 mmol) 3-bromo-9,9′-dimethyfluorene, 0.7 g (1 mmol) [1,1′-bis(diphenylphosphine)ferrocene] palladium dichloride, 0.5 g 2-bicyclohexylphosphine-2′,6′-dimethoxybiphenyl, 500 mL toluene, and 14.4 g (150 mmol) sodium tert-butoxide were added to a 1000 mL single-necked flask, and vacuumized for nitrogen exchange for 3 times, then the reaction was heated up to 90° C. for reacting for 5 h. The reaction was terminated at the end of the reaction. The flask was cooled to room temperature, and reaction liquid was separated, and organic phases were concentrated, then methanol was added and stirred for 1 h, and suction filtration was performed to obtain a faint yellow powder S4.

23 g (50 mmol) S4, 15 g (100 mmol) 2-phenyl-1-bromo-biphenyl, 0.9 g (1 mmol) tri(dibenzylidene acetone) dipalladium, 500 mL toluene were added to a 1000 mL single-necked flask, and vacuumized for nitrogen exchange for 3 times, and 0.5 mL tri-tert-butylphosphine toluene solution was added, and heated up to 110° C. for reaction for 12 h; at the end of the reaction, solvent was removed by evaporation, and silica-gel column chromatography was performed to obtain N418; M/Z theoretical value: 689, M/Z measured value: 690.

### Synthesis of the Compounds of Preferred Embodiment III

The synthetic routes of the compounds as shown in the Formulas (III-1), (III-2) and (III-3) of the present invention are as follows:

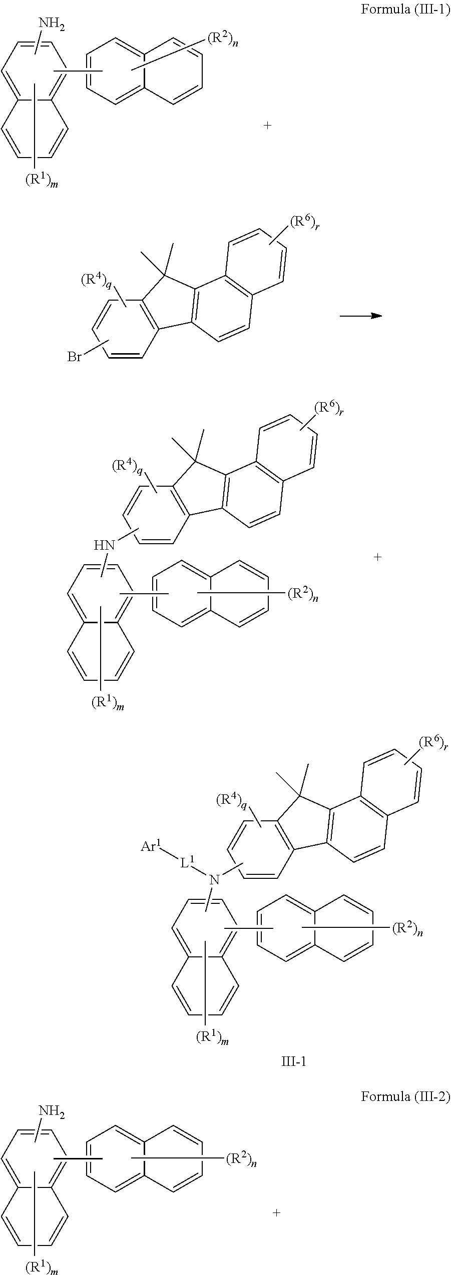

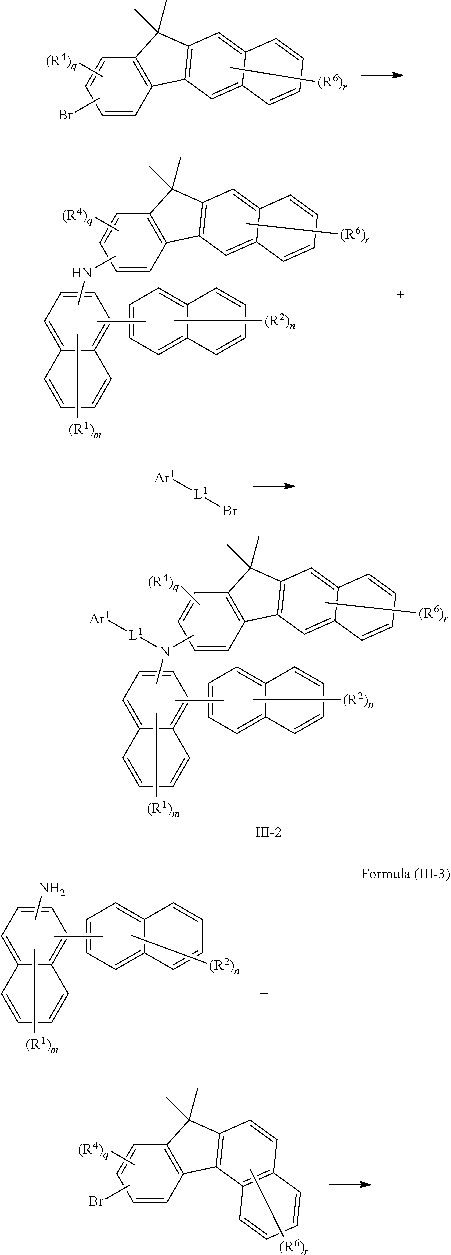

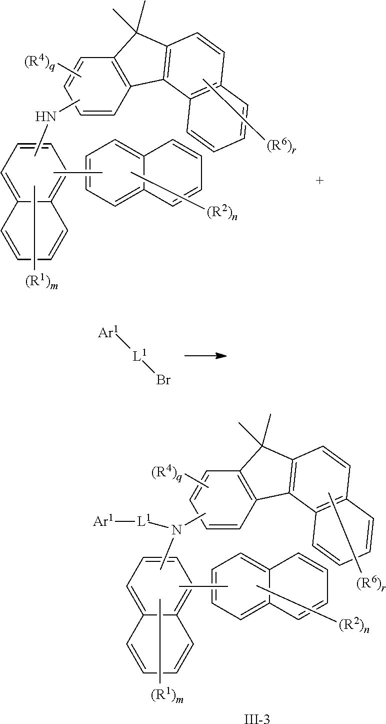

Multiple synthesis examples are set as examples below to describe the specific preparation methods of the above novel compounds of the present invention, but the preparation methods of the present invention are not limited to these synthesis examples.

### Synthesis Example 3-1: Synthesis of the Compound T1

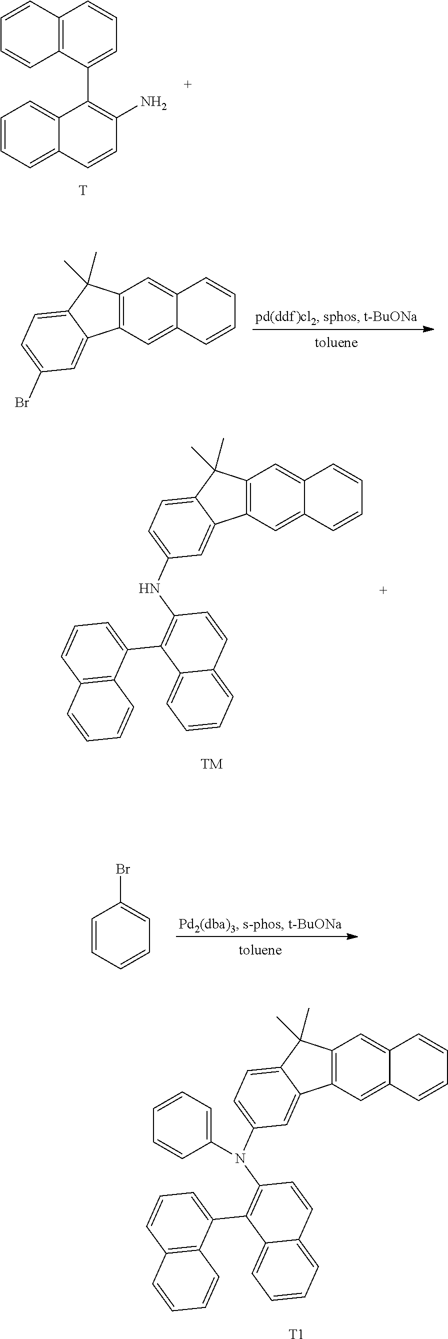

15 g (55.69 mmol) compound P, 18 g (55.69 mmol) 3-bromo-11,11-dimethyl-benzfluorene, 0.4 g (556.92 μmol) [1,1′-bis(diphenylphosphine)ferrocene] palladium dichloride (namely, Pd(dppf)Cl₂), 0.45 g (1.1 mmol) 2-bicyclohexylphosphine-2′,6′-dimethoxybiphenyl (namely, sphos), 200 mL toluene, and 16.06 g (167.08 mmol) sodium tert-butoxide (NaOBu-t) were added to a 500 mL single-necked flask, and vacuumized for nitrogen exchange for 3 times, then the reaction was heated up to 90° C. for reacting for 12 h. The reaction was terminated at the end of the reaction. The flask was cooled to room temperature, and reaction liquid was separated, and organic phases were concentrated, and silica-gel column chromatography was performed to obtain a compound PM. M/Z theoretical value: 511; M/Z measured value: 512.

20 g (39.09 mmol) compound PM, 7.9 g (50.82 mmol) bromobenzene, 0.71 g (781.78 μmol) tri(dibenzylidene acetone) dipalladium (namely, Pd₂(dba)₃), 0.64 g (1.56 mmol) 2-bicyclohexylphosphine-2′,6′-dimethoxy biphenyl, 300 mL toluene and 11.27 g (117.27 mmol) sodium tert-butoxide (NaOBu-t) were added to a 500 mL single-necked flask, and vacuumized for nitrogen exchange for 3 times, then the reaction was heated up to 110° C. for reacting for 10 h. The reaction was terminated at the end of the reaction. The flask was cooled to room temperature, and reaction liquid was separated, and organic phases were concentrated, then silica-gel column chromatography was performed to obtain a compound T1.M/Z theoretical value: 587; M/Z measured value: 588.

### Synthesis Example 3-2: Synthesis of the Compound T2

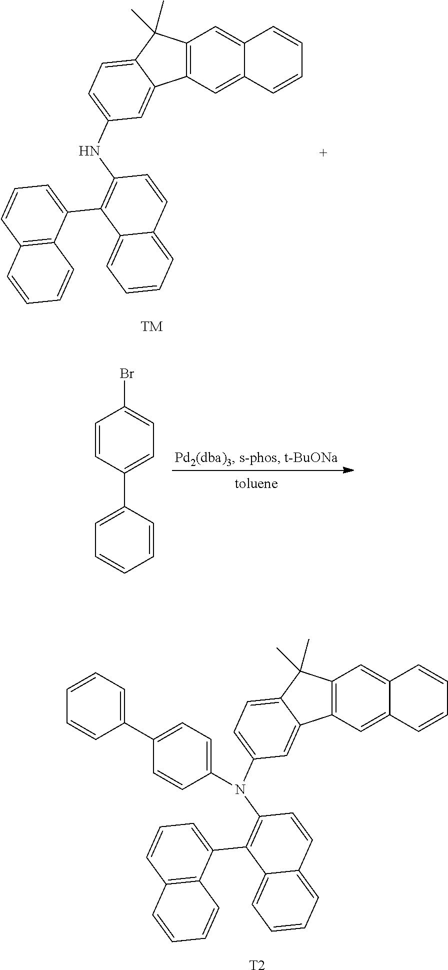

20 g (39.09 mmol) compound PM, 11.85 g (50.82 mmol) 4-bromobiphenyl, 0.71 g (781.78 μmol) tri(dibenzylidene acetone) dipalladium (namely, Pd₂(dba)₃), 0.64 g (1.56 mmol) 2-bicyclohexylphosphine-2′,6′-dimethoxy biphenyl (namely, sphos) 300 mL toluene and 11.27 g (117.27 mmol) sodium tert-butoxide (NaOBu-t) were added to a 500 mL single-necked flask, and vacuumized for nitrogen exchange for 3 times, then the reaction was heated up to 110° C. for reacting for 10 h. The reaction was terminated at the end of the reaction. The flask was cooled to room temperature, and reaction liquid was separated, and organic phases were concentrated, then silica-gel column chromatography was performed to obtain a compound T2.M/Z theoretical value: 663; M/Z measured value: 664.

### Synthesis Example 3-3: Synthesis of the Compound T11

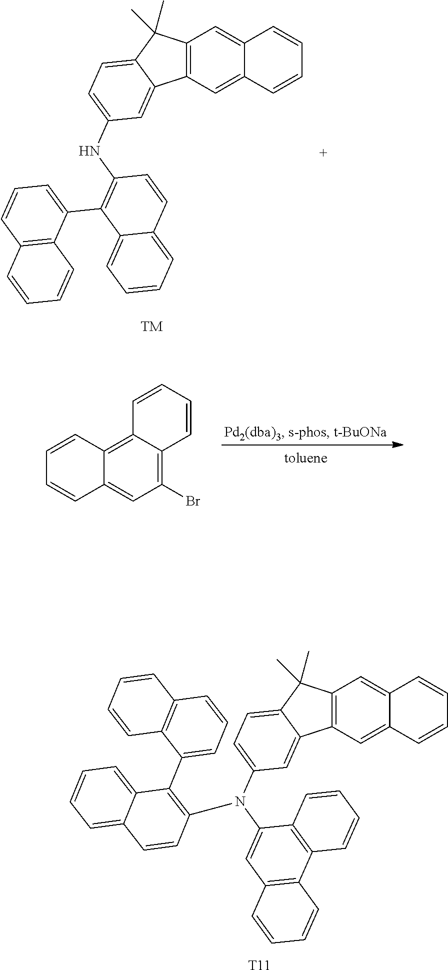

20 g (39.09 mmol) compound PM, 13.07 g (50.82 mmol) 9-bromophenanthrene, 0.71 g (781.78 μmol) tri(dibenzylidene acetone) dipalladium (namely, Pd₂(dba)₃), 0.64 g (1.56 mmol) 2-bicyclohexylphosphine-2′,6′-dimethoxy biphenyl (namely, sphos) 300 mL toluene and 11.27 g (117.27 mmol) sodium tert-butoxide (NaOBu-t) were added to a 500 mL single-necked flask, and vacuumized for nitrogen exchange for 3 times, then the reaction was heated up to 110° C. for reacting for 10 h. The reaction was terminated at the end of the reaction. The flask was cooled to room temperature, and reaction liquid was separated, and organic phases were concentrated, then silica-gel column chromatography was performed to obtain a compound T11.M/Z theoretical value: 687; M/Z measured value: 688.

### Synthesis Example 3-4: Synthesis of the Compound T12

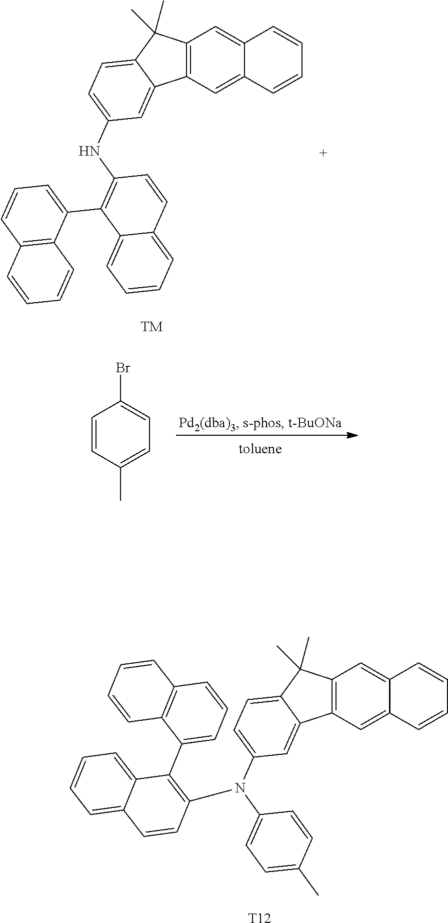

20 g (39.09 mmol) compound PM, 8.69 g (50.82 mmol) 1-bromo-4-methylbenzene, 0.71 g (781.78 μmol) tri(dibenzylidene acetone) dipalladium (namely, Pd₂(dba)₃), 0.64 g (1.56 mmol) 2-bicyclohexylphosphine-2′,6′-dimethoxy biphenyl (namely, sphos) 300 mL toluene and 11.27 g (117.27 mmol) sodium tert-butoxide (NaOBu-t) were added to a 500 mL single-necked flask, and vacuumized for nitrogen exchange for 3 times, then the reaction was heated up to 110° C. for reacting for 10 h. The reaction was terminated at the end of the reaction. The flask was cooled to room temperature, and reaction liquid was separated, and organic phases were concentrated, then silica-gel column chromatography was performed to obtain a compound T12.M/Z theoretical value: 601; M/Z measured value: 602.

### Synthesis Example 3-5: Synthesis of the Compound T81

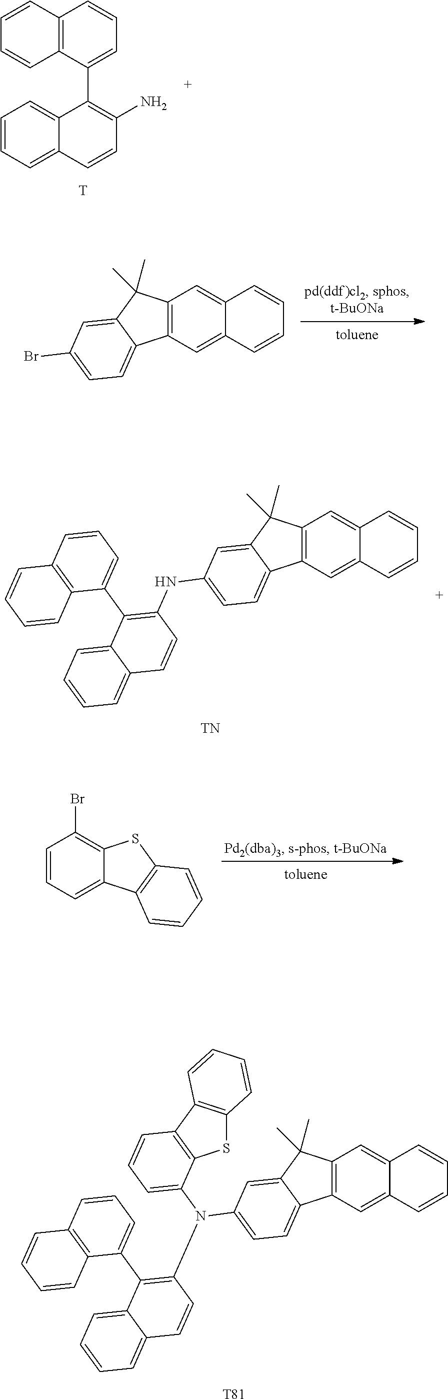

15 g (55.69 mmol) compound P, 18 g (55.69 mmol) 2-bromo-11,11-dimethyl-benzfluorene, 0.4 g (556.92 μmol) [1,1′-bis(diphenylphosphine)ferrocene] palladium dichloride (namely, Pd(dppf)Cl₂), 0.45 g (1.1 mmol) 2-biyclohexylphosphine-2′,6′-dimethoxybiphenyl (namely, sphos), 200 mL toluene, and 16.06 g (167.08 mmol) sodium tert-butoxide (NaOBu-t) were added to a 500 mL single-necked flask, and vacuumized for nitrogen exchange for 3 times, then the reaction was heated up to 90° C. for reacting for 12 h. The reaction was terminated at the end of the reaction. The flask was cooled to room temperature, and reaction liquid was separated, and organic phases were concentrated, and silica-gel column chromatography was performed to obtain a compound PN.M/Z theoretical value: 511; M/Z measured value: 512.

20 g (39.09 mmol) compound PM, 13.37 g (50.82 mmol) 4-bromodibenzothiophene, 0.71 g (781.78 μmol) tri(dibenzylidene acetone) dipalladium (namely, Pd₂(dba)₃), 0.64 g (1.56 mmol) 2-bicyclohexylphosphine-2′,6′-dimethoxy biphenyl (namely, sphos) 300 mL toluene and 11.27 g (117.27 mmol) sodium tert-butoxide (NaOBu-t) were added to a 500 mL single-necked flask, and vacuumized for nitrogen exchange for 3 times, then the reaction was heated up to 110° C. for reacting for 10 h. The reaction was terminated at the end of the reaction. The flask was cooled to room temperature, and reaction liquid was separated, and organic phases were concentrated, then silica-gel column chromatography was performed to obtain a compound T81.M/Z theoretical value: 693; M/Z measured value: 694.

### Synthesis Example 3-6: Synthesis of the Compound T163

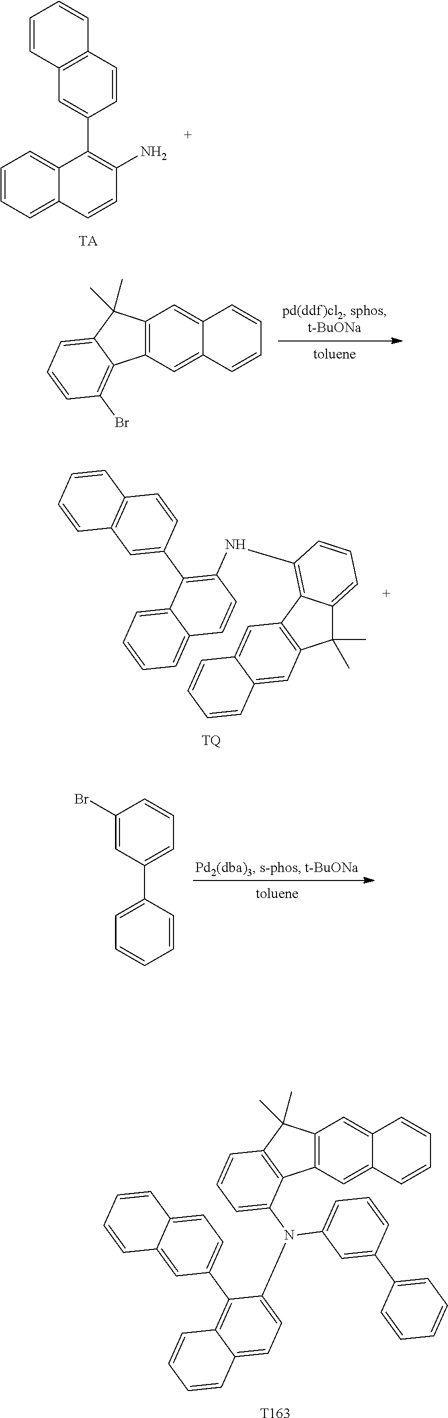

15 g (55.69 mmol) compound PA, 18 g (55.69 mmol) 4-bromo-11,11-dimethyl-benzfluorene, 0.4 g (556.92 μmol) [1,1′-bis(diphenylphosphine)ferrocene] palladium dichloride (namely, Pd(dppf)Cl₂), 0.45 g (1.1 mmol) 2-bicyclohexylphosphine-2′,6′-dimethoxybiphenyl (namely, sphos), 200 mL toluene, and 16.06 g (167.08 mmol) sodium tert-butoxide (NaOBu-t) were added to a 500 mL single-necked flask, and vacuumized for nitrogen exchange for 3 times, then the reaction was heated up to 90° C. for reacting for 12 h. The reaction was terminated at the end of the reaction. The flask was cooled to room temperature, and reaction liquid was separated, and organic phases were concentrated, and silica-gel column chromatography was performed to obtain a compound PN.M/Z theoretical value: 511; M/Z measured value: 512.

20 g (39.09 mmol) compound PQ, 11.85 g (50.82 mmol) m-bromotoluene, 0.71 g (781.78 μmol) tri(dibenzylidene acetone) dipalladium (namely. Pd₂(dba)₃), 0.64 g (1.56 mol) 2-bicyclohexylphosphine-2′,6′-dimethoxy biphenyl (namely, sphos) 300 mL toluene and 11.27 g (117.27 mmol) sodium tert-butoxide (NaOBu-t) were added to a 500 mL single-necked flask, and vacuumized for nitrogen exchange for 3 times, then the reaction was heated up to 110° C. for reacting for 10 h. The reaction was terminated at the end of the reaction. The flask was cooled to room temperature, and reaction liquid was separated, and organic phases were concentrated, then silica-gel column chromatography was performed to obtain a compound T163.M/Z theoretical value: 663; M/Z measured value: 664.

### Synthesis Example 3-7: Synthesis of the Compound T170

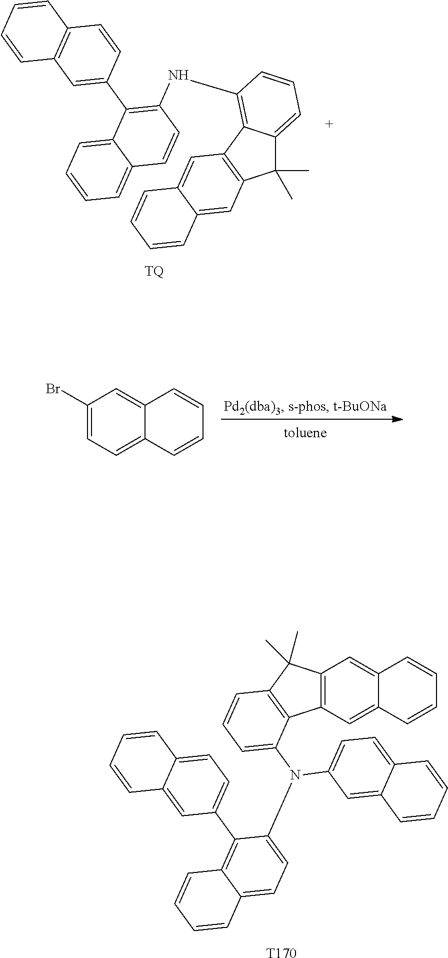

20 g (39.09 mmol) compound PQ, 10.52 g (50.82 mmol) 2-bromonaphthalene, 0.71 g (781.78 μmol) tri(dibenzylidene acetone) dipalladium (namely, Pd₂(dba)₃), 0.64 g (1.56 mmol) 2-bicyclohexylphosphine-2′,6′-dimethoxy biphenyl (namely, sphos) 300 mL toluene and 11.27 g (117.27 mmol) sodium tert-butoxide (NaOBu-t) were added to a 500 mL single-necked flask, and vacuumized for nitrogen exchange for 3 times, then the reaction was heated up to 110° C. for reacting for 10 h. The reaction was terminated at the end of the reaction. The flask was cooled to room temperature, and reaction liquid was separated, and organic phases were concentrated, then silica-gel column chromatography was performed to obtain a compound T170.M/Z theoretical value: 637; M/Z measured value: 638.

### Synthesis Example 3-8: Synthesis of the Compound T232

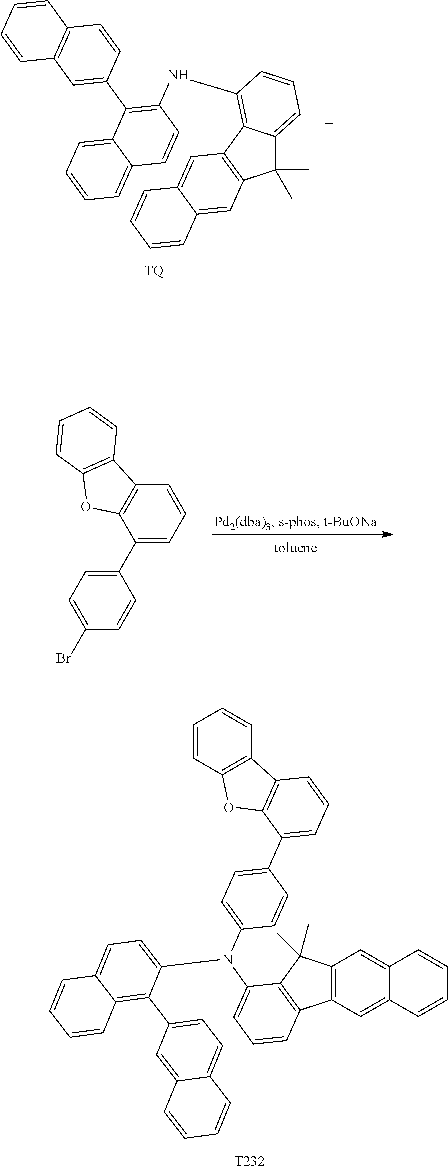

20 g (39.09 mmol) compound PQ, 16.42 g (50.82 mmol) 4-(4-bromophenyl-dibenzofuran, 0.71 g (781.78 μmol) tri(dibenzylidene acetone) dipalladium (namely, Pd₂(dba)₃), 0.64 g (1.56 mmol) 2-bicyclohexylphosphine-2′,6′-dimethoxy biphenyl (namely, sphos) 300 mL toluene and 11.27 g (117.27 mmol) sodium tert-butoxide (NaOBu-t) were added to a 500 mL single-necked flask, and vacuumized for nitrogen exchange for 3 times, then the reaction was heated up to 110° C. for reacting for 10 h. The reaction was terminated at the end of the reaction. The flask was cooled to room temperature, and reaction liquid was separated, and organic phases were concentrated, then silica-gel column chromatography was performed to obtain a compound T232. M/Z theoretical value: 753; M/Z measured value: 754.

The compounds of the present invention will be specifically applied in an organic electroluminescent device to test actual operational performance to display and verify the technical effects and advantages of the present invention.

### Devices Utilizing the Compounds of Preferred Embodiment I

### Example 1-1

This example provides an organic electroluminescent device, and the specific preparation process is as follows:

a glass pane coated with an ITO transparent conducting layer was subjected to ultrasonic treatment in a commercial detergent, washed in deionized water, and subjected to ultrasonic degreasing in a mixed solvent of acetone: ethanol, and baked in a clean environment till water content was removed completely; then washed with UV-light and ozone, and the surface thereof was bombarded with a low-energy cationic beam;

the above glass substrate with an anode was put to a vacuum chamber, and vacuumized to be less than 1×10⁻⁵ Pa; the above anode coating film was evaporated with an HT-4:HI-3 (97/3, w/w) mixture under vacuum as a hole injection layer, where the evaporation rate was 0.1 nm/s and the evaporation coating thickness was 10 nm;

HT-4 was evaporated above the hole injection layer under vacuum as a hole transport layer of the device, where the evaporation rate was 0.1 nm/s and the total evaporation coating thickness was 60 nm;

the compound p1 synthesized in the synthesis example 1-1 was evaporated above the hole transport layer under vacuum as an electron blocking layer material of the device, where the evaporation rate was 0.1 nm/s and the total evaporation coating thickness was 60 nm;

a luminescent layer was evaporated above the electron blocking layer under vacuum; the luminescent layer includes a host material and a dyeing material; a multi-source co-evaporation method was used to adjust the evaporation rate of the host material GPH-59 to 0.1 nm/s; the evaporation rate of the dye RPD-8 was set by a ratio of 3%, and the total evaporation coating thickness was 40 nm;

an electron transport layer (material: ET-46) was evaporated above the luminescent layer under vacuum, and set by a ratio of 50%, and ET-57 was set by a ratio of 50%, where the evaporation rate was 0.1 nm/s and the total evaporation coating thickness was 25 nm:

LF with a thickness of 0.5 nm, as an electron injection layer, was evaporated above the electron transport layer (ETL) under vacuum and an Al layer with a thickness of 150 nm served as a cathode of the device.

### Examples 1-2 to 1-25

The preparing process of Examples 1-2 to 1-25 is the same that in Example 1-1, and what is different is that the compound P1 of the electron blocking layer material is respectively replaced with the compounds as shown in Table 1.

### Comparative Examples 1-1 to 1-2

The preparing process of Comparative Examples 1-1 to 1-2 is the same that in Example 1-1, and what is different is that the compound P1 of the electron blocking layer material is respectively replaced with the compounds R-1 and R-2; the compound used in Comparative Examples 1-1 to 1-2 has the following structure:

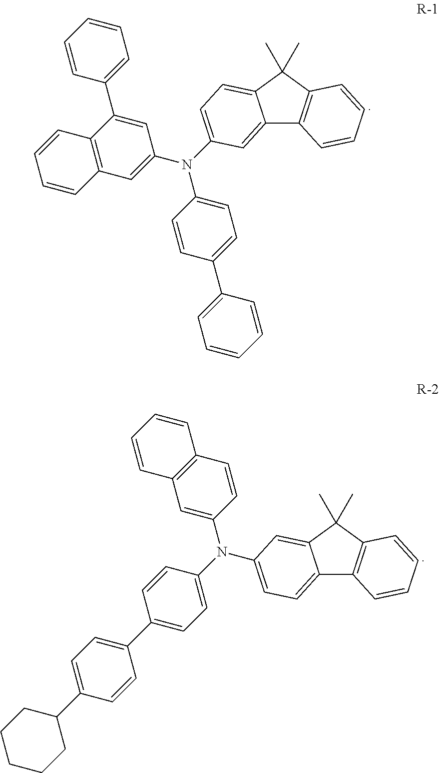 [SMILES: CC1(C)c2ccccc2-c2cc(N(c3ccc(-c4ccccc4)cc3)c3cc(-c4ccccc4)c4ccccc4c3)ccc21 ; CC1(C)c2ccccc2-c2ccc(N(c3ccc(-c4ccc(C5CCCCC5)cc4)cc3)c3ccc4ccccc4c3)cc21]

performance measurement is performed on the organic electroluminescent device prepared by the above process below:

(1) at a same luminance value, a digital source-meter (Keithley2400) and a luminance meter (ST-86LA luminance meter, Beijing Normal University Photoelectric Instrument Plant) were used to measure the driving voltage, current efficiency and service life of the organic electroluminescent device prepared in Examples 1-1 to 1-25 and Comparative Examples 1-1 to 1-2. Specifically, voltage was increased at a rate of 0.1V/s, when the luminance of the organic electroluminescent device was up to 5000 cd/m², the voltage was measured as the driving voltage, and electric current density was measured at this time simultaneously; the ratio of the luminance to the electric current density was the current efficiency;

(2) life test of LT95 was as follows: a luminance meter was used to keep a constant current under a luminance value of 5000 cd/m²; the time when the luminance of the organic electroluminescent device dropped to 4750 cd/m² was measured with a unit of hour.

The test results were shown in table 1.

TABLE 1

| | | Desired | | Current |
|---|---|---|---|---|
| | Hole-transport | luminance | Voltage | efficiency |
| | layer material | cd/m | V | cd/A |
| Comparative | R-1 | 5000.00 | 5.3 | 10.2 |
| Example 1-1 | | | | |
| Comparative | R-2 | 5000.00 | 5.8 | 8.9 |
| Example 1-2 | | | | |
| Example 1-1 | P1 | 5000.00 | 4.8 | 15 |
| Example 1-2 | P3 | 5000.00 | 4.6 | 16.2 |
| Example 1-3 | P11 | 5000.00 | 4.6 | 16.7 |
| Example 1-4 | P31 | 5000.00 | 5.0 | 13 |
| Example 1-5 | P37 | 5000.00 | 4.7 | 15 |
| Example 1-6 | P39 | 5000.00 | 4.7 | 14.2 |
| Example 1-7 | P42 | 5000.00 | 4.8 | 13.2 |
| Example 1-8 | P61 | 5000.00 | 4.6 | 17.1 |
| Example 1-9 | P62 | 5000.00 | 4.7 | 12.8 |
| Example 1-10 | P73 | 5000.00 | 4.8 | 16.4 |
| Example 1-11 | P75 | 5000.00 | 4.9 | 14.7 |
| Example 1-12 | P97 | 5000.00 | 4.8 | 18 |
| Example 1-13 | P109 | 5000.00 | 4.6 | 15.6 |
| Example 1-14 | P121 | 5000.00 | 4.7 | 16.9 |
| Example 1-15 | P133 | 5000.00 | 4.7 | 14.5 |
| Example 1-16 | P173 | 5000.00 | 4.8 | 16.1 |
| Example 1-17 | P179 | 5000.00 | 4.9 | 15.4 |
| Example 1-18 | P189 | 5000.00 | 4.8 | 14 |
| Example 1-19 | P198 | 5000.00 | 4.5 | 15.3 |
| Example 1-20 | P209 | 5000.00 | 4.9 | 15.5 |
| Example 1-21 | P224 | 5000.00 | 4.6 | 14.5 |
| Example 1-22 | P229 | 5000.00 | 4.6 | 14.8 |
| Example 1-23 | P269 | 5000.00 | 4.9 | 15.8 |
| Example 1-24 | P278 | 5000.00 | 4.9 | 13.8 |
| Example 1-25 | P287 | 5000.00 | 5.0 | 14.1 |

It can be seen from the results of Table 1 that when the compounds of the present invention are used as the hole-transport material of the organic electroluminescent device, and when the luminance is up to 5000 cd/m², the driving voltage is as low as 5.0 V below, and the current efficiency is up to 12.8 cd/A above; compared with the Comparative Examples 1-1 to 1-2, the compounds the present invention may effectively reduce the driving voltage, improve the current efficiency and thus, is a kind of electron blocking material with good performances. The reason has been not clear, but it is presumed as follows: compared with the compound R-1 of Comparative Example 1-1, when the compounds in Examples 1-1 to 1-25 of the present invention are used as the electron blocking material of the organic electroluminescent device, because there is a cycloalkyl group substituted in a specific position and there is an aromatic substituent in an orthortho position of amido on a naphthalene ring, molecules may be promoted to be spread out on a plane of the device, which induces the subsequently deposited molecules on the luminescent layer also to be piled in such a plane space way. The luminescent molecules piled in a spreading way are beneficial to the improvement of optical extraction efficiency, thereby promoting the current efficiency. Due to lack of an aromatic substituent in the orthortho position of amido only, the compound R-2 used in the Comparative Example 1-2 may not achieve high efficiency, and the voltage is staying at a high level. Thus, it can be seen that the molecules may not achieve the beneficial molecular arrangement possessed by the compounds of the present invention. The above analysis is enough to show that the unique molecular structure of the compounds of the present invention, is the crucial to achieve the outstanding performance of the devices in the examples. When the luminance of the organic electroluminescent device utilizing the compounds of the present invention is up to 5000 cd/m², the driving voltage is as low as 5.0 V and below, and the current efficiency is up to 12.8 cd/A and above, and LT95 is up to 21 h and above.

### Devices Utilizing the Compounds of Preferred Embodiment II

### Example 2-1

This example provides an organic electroluminescent device, and the specific preparation process is as follows:

a glass pane coated with an ITO transparent conducting layer was subjected to ultrasonic treatment in a commercial detergent, washed in deionized water, and subjected to ultrasonic degreasing in a mixed solvent of acetone: ethanol, and baked in a clean environment till water was removed completely; then washed with UV-light and ozone, and the surface was bombarded with a low-energy cationic beam;

the above glass substrate with an anode was put to a vacuum chamber, and vacuumized to be less than 1×10⁻⁵ Pa; the above anode coating film was evaporated with HI-3 under vacuum as a hole injection layer, where the evaporation rate was 0.1 nm/s and the evaporation coating thickness was 10 nm;

the compound N1 synthesized in the synthesis example 2-1 was evaporated above the hole injection layer under vacuum as a hole transport layer of the device, where the evaporation rate was 0.1 nm/s and the total evaporation coating thickness was 80 nm;

HT-14 was evaporated above the hole transport layer under vacuum as an electron blocking layer of the device, where the evaporation rate was 0.1 nm/s and the total evaporation coating thickness was 80 nm;

a luminescent layer of the device was evaporated above the electron blocking layer under vacuum; the luminescent layer includes a host material and a dyeing material; a multi-source co-evaporation method was used to adjust the evaporation rate of the host material GPH-59 to 0.1 nm/s; the evaporation rate of the dye RPD-8 was set by a ratio of 3%, and the total evaporation coating thickness was 30 nm;

an electron transport layer (material: ET-46) was evaporated above the luminescent layer under vacuum, and set by a ratio of 50%, and ET-57 was set by a ratio of 50%, where the evaporation rate was 0.1 nm/s and the total evaporation coating thickness was 30 nm;

LF with a thickness of 0.5 nm, as an electron injection layer, was evaporated above the electron transport layer (ETL) under vacuum, and an Al layer with a thickness of 150 nm served as a cathode of the device.

### Examples 2-2 to 2-33 and Comparative Examples 2-1 to 2-4

The preparing process of Examples 2-2 to 2-33 and Comparative Examples 2-1 to 2-4 is the same that in Example 2-1, and what is different is that the compound N1 is replaced with the compounds as shown in Table 2, as the hole-transport material.

The hole-transport materials EMT-1 to EMT-4 in Comparative Examples 2-1 to 2-4 have the following structure:

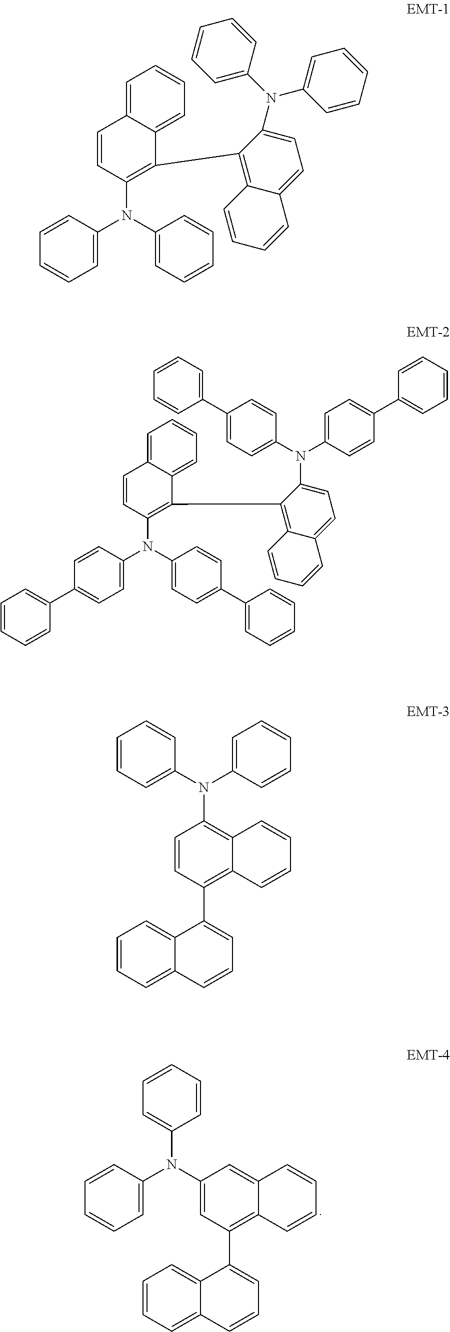 [SMILES: c1ccc(N(c2ccccc2)c2ccc3ccccc3c2-c2c(N(c3ccccc3)c3ccccc3)ccc3ccccc23)cc1 ; c1ccc(-c2ccc(N(c3ccc(-c4ccccc4)cc3)c3ccc4ccccc4c3-c3c(N(c4ccc(-c5ccccc5)cc4)c4ccc(-c5ccccc5)cc4)ccc4ccccc34)cc2)cc1 ; c1ccc(N(c2ccccc2)c2ccc(-c3cccc4ccccc34)c3ccccc23)cc1 ; c1ccc(N(c2ccccc2)c2cc(-c3cccc4ccccc34)c3ccccc3c2)cc1]

performance measurement is performed on the organic electroluminescent device prepared in Examples 2-1 to 2-33 and Comparative Examples 2-1 to 2-4 below:

at a same luminance value, a digital source-meter and a luminance meter were used to measure the driving voltage, current efficiency and service life of the organic electroluminescent device prepared in Examples 2-1 to 2-33 and Comparative Examples 2-1 to 2-4. Specifically, voltage was increased at a rate of 0.1 V/s, when the luminance of the organic electroluminescent device was up to 3000 cd/m², the voltage was measured as the driving voltage, and electric current density was measured at this time simultaneously; the ratio of the luminance to the electric current density was the current efficiency; LTO5 life test was as follows: a luminance meter was used to keep a constant current under a luminance value of 5000 cd/m2; the time when the luminance of the organic electroluminescent device dropped to 4750 cd/m² was measured with a unit of hour. The measured results were shown in table 2.

TABLE 2

| | hole- | | | | |
|---|---|---|---|---|---|
| | transport | Desired | | Current | Service |
| | layer | luminance | Voltage | efficiency | life |
| | material | cd/m² | V | cd/A | (LT95) h |
| Comparative | EMT-1 | 3000 | 5.7 | 7.5 | 60 |
| Example 2-1 | | | | | |
| Comparative | EMT-2 | 3000 | 5.5 | 8.1 | 58 |
| Example 2-2 | | | | | |
| Comparative | EMT-3 | 3000 | 4.6 | 9.3 | 77 |
| Example 2-3 | | | | | |
| Comparative | EMT-4 | 3000 | 4.7 | 10.2 | 86 |
| Example 2-4 | | | | | |
| Example 2-1 | N1 | 3000 | 3.1 | 15 | 196 |
| Example 2-2 | N13 | 3000 | 3.4 | 13.5 | 201 |
| Example 2-3 | N34 | 3000 | 3.2 | 14.2 | 187 |
| Example 2-4 | N63 | 3000 | 3.5 | 13 | 200 |
| Example 2-5 | N93 | 3000 | 3.4 | 10.5 | 162 |
| Example 2-6 | N94 | 3000 | 3.2 | 16 | 230 |
| Example 2-7 | N100 | 3000 | 3.4 | 10.8 | 188 |
| Example 2-8 | N120 | 3000 | 3.2 | 13.6 | 195 |
| Example 2-9 | N134 | 3000 | 3.2 | 13.6 | 196 |
| Example 2-10 | N147 | 3000 | 3.1 | 12 | 200 |
| Example 2-11 | N170 | 3000 | 3.2 | 15 | 194 |
| Example 2-12 | N176 | 3000 | 3.2 | 16 | 210 |
| Example 2-13 | N191 | 3000 | 3.1 | 16.2 | 198 |
| Example 2-14 | N314 | 3000 | 3.3 | 15.1 | 231 |
| Example 2-15 | N325 | 3000 | 3.3 | 11 | 179 |
| Example 2-16 | N331 | 3000 | 3.3 | 14.5 | 185 |
| Example 2-17 | N337 | 3000 | 3.3 | 15.5 | 187 |
| Example 2-18 | N371 | 3000 | 3.1 | 16.3 | 163 |
| Example 2-19 | N372 | 3000 | 3.2 | 15.4 | 187 |
| Example 2-20 | N373 | 3000 | 3.2 | 16.2 | 213 |
| Example 2-21 | N374 | 3000 | 3.4 | 17.1 | 193 |
| Example 2-22 | N375 | 3000 | 3.1 | 14.8 | 152 |
| Example 2-23 | N376 | 3000 | 3.1 | 13.4 | 183 |
| Example 2-24 | N377 | 3000 | 3.2 | 18.4 | 178 |
| Example 2-25 | N378 | 3000 | 3.5 | 14.5 | 195 |
| Example 2-26 | N379 | 3000 | 3.3 | 14.1 | 164 |
| Example 2-27 | N380 | 3000 | 3.2 | 15 | 185 |
| Example 2-28 | N381 | 3000 | 3.1 | 14.7 | 178 |
| Example 2-29 | N382 | 3000 | 3.5 | 15.8 | 169 |
| Example 2-30 | N383 | 3000 | 3.1 | 16.1 | 186 |
| Example 2-31 | N387 | 3000 | 3.0 | 16 | 202 |
| Example 2-32 | N389 | 3000 | 3.1 | 18.3 | 197 |
| Example 2-33 | N396 | 3000 | 3.1 | 17.7 | 226 |

It can be seen from the results of Table 2 that when the compounds in Examples 2-1 to 2-33 of the present invention are used as the hole-transport material of the organic electroluminescent device, and when the luminance is up to 3000 cd/m², the driving voltage is as low as 3.5 V below, and the current efficiency is up to 10.5 cd/A above; LT95 is up to 152 h above. Therefore, the compounds of the present invention may effectively reduce the driving voltage, improve the current efficiency and prolong the service life of the device, and thus is a kind of electron blocking material with good performances. In contrast to this, the organic electroluminescent devices, in which the compounds in Comparatives Examples 2-1 to 2-4 were used as hole-transport materials have different levels of shortages in driving voltage, current efficiency, service life and other aspects. The reason has been not clear, but it is presumed as follows: in the molecular structure of compounds EMT 1 and EMT-2 in Comparative Examples 2-1 and 2-2, R² is arylamido; and in the molecular structure of compounds EMT-3 and EMT-4 in Comparative Examples 2-3 and 2-4, the arylamido on the naphthalene ring and naphthyl are not located in the orthortho position. Therefore, these compounds may not accord with the definition requirement of claim 1 and thus may not achieve the technical effect of the present invention.

### Examples 2-34

This example provides an organic electroluminescent device, and the specific preparation process is as follows:

a glass pane coated with an ITO transparent conducting layer was subjected to ultrasonic treatment in a commercial detergent, washed in deionized water, and subjected to ultrasonic degreasing in a mixed solvent of acetone: ethanol, and baked in a clean environment till water was removed completely; then washed with UV-light and ozone, and the surface thereof was bombarded with a low-energy cationic beam;

the above glass substrate with an anode was put to a vacuum chamber, and vacuumized to be less than 1×10⁻⁵ Pa; the above anode coating film was evaporated with an HI-3 under vacuum as a hole injection layer, where the evaporation rate was 0.1 nm/s and the evaporation coating thickness was 10 nm;

HT-4 was evaporated above the hole injection layer under vacuum as a hole transport layer of the device, where the evaporation rate was 0.1 nm/s and the total evaporation coating thickness was 80 nm;

the compound N1 synthesized in the synthesis example 1 was evaporated above the hole transport layer under vacuum as an electron blocking layer of the device, where the evaporation rate was 0.1 nm/s and the total evaporation coating thickness was 80 nm;

a luminescent layer of the device was evaporated above the electron blocking layer under vacuum; the luminescent layer includes a host material and a dyeing material; and a multi-source co-evaporation method was used to adjust the evaporation rate of the host material GPH-59 to 0.1 nm/s; the evaporation rate of the dye RPD-8 was set by a ratio of 3%, and the total evaporation coating thickness was 30 nm;

an electron transport layer (material: ET-46) was evaporated above the luminescent layer under vacuum, and set by a ratio of 50%, and ET-57 was set by a ratio of 50%, where the evaporation rate was 0.1 nm/s and the total evaporation coating thickness was 30 nm;

LF with a thickness of 0.5 nm, as an electron injection layer, was evaporated above the electron transport layer (ETL) under vacuum r, and an Al layer with a thickness of 150 nm served as a cathode of the device.

### Examples 2-35 to 2-71 and Comparative Examples 2-5 to 2-8

The preparing process of Examples 2-35 to 2-71 and Comparative Examples 2-5 to 2-8 is the same that in Example 2-34, and what is different is that the compound N1 is replaced with the compounds as shown in Table 3 as the hole-transport material.

Performance measurement is performed on the organic electroluminescent device prepared in Examples 2-34 to 2-71 and Comparative Examples 2-5 to 2-8 below:

at a same luminance value, a digital source-meter and a luminance meter were used to measure the driving voltage, current efficiency and service life of the organic electroluminescent device prepared in Examples 2-34 to 2-71 and Comparative Examples 2-5 to 2-8. Specifically, voltage was increased at a rate of 0.1 V/s, when the luminance of the organic electroluminescent device was up to 3000 cd/m², the voltage was measured as the driving voltage, and electric current density was measured at this time simultaneously; the ratio of the luminance to the electric current density was the current efficiency; LT95 life test was as follows: a luminance meter was used to keep a constant current under a luminance value of 5000 cd/m²: the time when the luminance of the organic electroluminescent device dropped to 4750 cd/m² was measured with a unit of hour. The measured results were shown in Table 3.

TABLE 3

| | Electron | | | | |
|---|---|---|---|---|---|
| | blocking | Desired | | Current | Service |
| | layer | luminance | Voltage | efficiency | life |
| | material | cd/m² | V | cd/A | (LT95) h |
| Comparative | EMT-1 | 3000 | 5.6 | 7.6 | 66 |
| Example 2-5 | | | | | |
| Comparative | EMT-2 | 3000 | 5.4 | 8.9 | 93 |
| Example 2-6 | | | | | |
| Comparative | EMT-3 | 3000 | 5.0 | 8.2 | 87 |
| Example 2-7 | | | | | |
| Comparative | EMT-4 | 3000 | 5.3 | 8.5 | 70 |
| Example 2-8 | | | | | |
| Examples 2-34 | N1 | 3000 | 3.7 | 13 | 214 |
| Examples 2-35 | N13 | 3000 | 3.4 | 16.3 | 220 |
| Examples 2-36 | N34 | 3000 | 3.3 | 14.2 | 173 |
| Examples 2-37 | N63 | 3000 | 3.8 | 14 | 230 |
| Examples 2-38 | N93 | 3000 | 3.6 | 12.5 | 180 |
| Examples 2-39 | N94 | 3000 | 3.5 | 18 | 250 |
| Examples 2-40 | N100 | 3000 | 3.4 | 16 | 190 |
| Examples 2-41 | N120 | 3000 | 3.2 | 17 | 241 |
| Examples 2-42 | N134 | 3000 | 3.5 | 17.5 | 196 |
| Examples 2-43 | N147 | 3000 | 3.2 | 19 | 243 |
| Examples 2-44 | N170 | 3000 | 3.6 | 17 | 201 |
| Examples 2-45 | N176 | 3000 | 3.8 | 16.5 | 198 |
| Examples 2-46 | N191 | 3000 | 3.5 | 19 | 184 |
| Examples 2-47 | N314 | 3000 | 3.6 | 15.4 | 192 |
| Examples 2-48 | N325 | 3000 | 3.4 | 17 | 188 |
| Examples 2-49 | N331 | 3000 | 3.2 | 16.8 | 211 |
| Examples 2-50 | N337 | 3000 | 3.5 | 17.4 | 184 |
| Examples 2-51 | N371 | 3000 | 3.7 | 18.9 | 199 |
| Examples 2-52 | N372 | 3000 | 3.5 | 15.9 | 167 |
| Examples 2-53 | N373 | 3000 | 3.1 | 20 | 238 |
| Examples 2-54 | N374 | 3000 | 3.3 | 19 | 223 |
| Examples 2-55 | N375 | 3000 | 3.2 | 17 | 176 |
| Examples 2-56 | N376 | 3000 | 3.0 | 14 | 169 |
| Examples 2-57 | N377 | 3000 | 3.8 | 21 | 250 |
| Examples 2-58 | N378 | 3000 | 3.6 | 22 | 199 |
| Examples 2-59 | N379 | 3000 | 3.2 | 19.6 | 189 |
| Examples 2-60 | N380 | 3000 | 3.3 | 18 | 235 |
| Examples 2-61 | N381 | 3000 | 3.4 | 16.9 | 174 |
| Examples 2-62 | N382 | 3000 | 3.2 | 18 | 197 |
| Examples 2-63 | N383 | 3000 | 3.0 | 19 | 186 |
| Examples 2-64 | N387 | 3000 | 3.0 | 18.9 | 233 |
| Examples 2-65 | N389 | 3000 | 3.1 | 19.3 | 241 |
| Examples 2-66 | N396 | 3000 | 3.0 | 21 | 231 |
| Examples 2-67 | N405 | 3000 | 3.3 | 19 | 210 |
| Examples 2-68 | N406 | 3000 | 3.4 | 18.5 | 198 |
| Examples 2-69 | N409 | 3000 | 3.2 | 19.2 | 180 |
| Examples 2-70 | N414 | 3000 | 3.3 | 20 | 179 |
| Examples 2-71 | N418 | 3000 | 3.5 | 17.8 | 222 |

It can be seen from the results of Table 3 that when the compounds in Examples 2-34 to 2-71 of the present invention are used as the electron blocking layer materials of the organic electroluminescent device, and when the luminance is up to 3000 cd/m², the driving voltage is as low as 3.8 V below, and the current efficiency is up to 12.5 cd/A above; LT95 is up to 167 h above. Therefore, the compounds of the present invention may effectively reduce the driving voltage, improve the current efficiency and thus, prolong the service life of the device, and thus is a kind of electron blocking material with good performances. In contrast to this, the organic electroluminescent devices in which the compounds in Comparatives Examples 2-5 to 2-8 were used as electron blocking layer materials, have different levels of shortages in driving voltage, current efficiency, service life and other aspects. The reason has been not clear, but it is presumed as follows: in the molecular structure of compounds EMT-1 and EMT-2 in Comparative Examples 2-5 and 2-6, R² is arylamido; and in the molecular structure of compounds EMT-3 and EMT-4 in Comparative Examples 2-7 and 2-8, the arylamido on the naphthalene ring and naphthyl are not located in the orthortho position. Therefore, these compounds may not accord with the definition requirement of claim 1 and thus may not achieve the technical effect of the present invention.

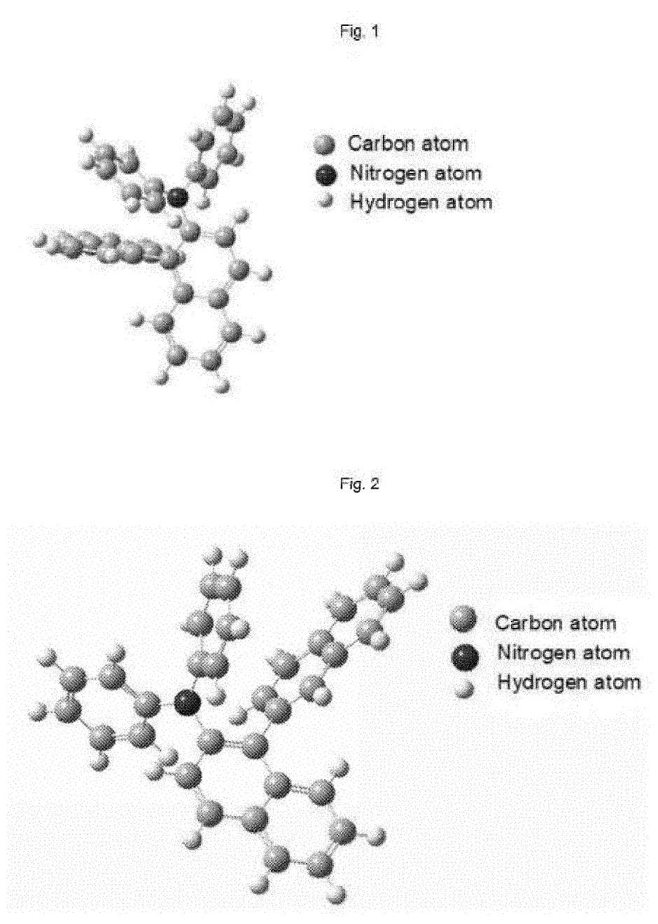

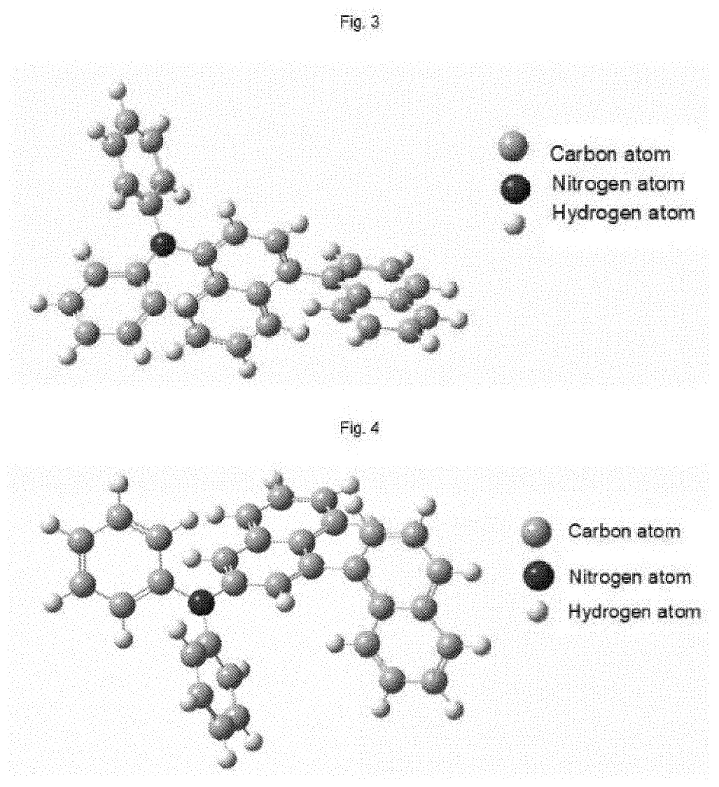

It can be seen from the above results that the above compounds may be used as hole transport (HTL) materials, and also used as electron blocking layer (EBL) materials in combination with other hole-transport materials. When the above compounds are used as hole-transport materials, voltage of all the examples reduces significantly, and performance and service life are improved obviously. When the above compounds are in combination with other hole-transport materials for use, voltage of the device of all the examples increases slightly, and efficiency and service life of the device are further improved substantially. By the comparison between the molecular structure modeling (FIGS. 1 and 2) of the compounds of the present invention and the molecular structure modeling (FIGS. 3 and 4) of the compounds in Comparative Examples, it can be seen that the dinaphthalene compounds where naphthyl is substituted in an orthortho position provided by the present invention may not only reserve the large plane structure π of the compounds (e.g., EMT-3 to EMT-4) in Comparative Examples, but also may effectively change the molecular space structure, beneficial to improving molecule accumulation within a film. Therefore, compared with Comparative Examples, the materials of the present invention have better efficiency. Further, Gaussian computation indicates that the orthortho position substitution limits the rotation of an aromatic ring on N atoms, thus enhancing the stability of such material. Therefore, the material has a longer service life.

### Devices Utilizing the Compounds of Preferred Embodiment III

### Example 3-1

This example provides an organic electroluminescent device, and the specific preparation process is as follows:

a glass pane coated with an ITO transparent conducting layer was subjected to ultrasonic treatment in a commercial detergent, washed in deionized water, and subjected to ultrasonic degreasing in a mixed solvent of acetone: ethanol, and baked in a dean environment till water was removed completely; then washed with UV-light and ozone, and the surface thereof was bombarded with a low-energy cationic beam;

the above glass substrate with an anode was put to a vacuum chamber, and vacuumized to be less than 1×10⁻⁵ Pa; the above anode coating film was evaporated with HI-3 under vacuum as a hole injection layer, where the evaporation rate was 0.1 nm/s and the evaporation coating thickness was 10 nm;

HT-4 was evaporated above the hole injection layer under vacuum as a hole transport layer of the device, where the evaporation rate was 0.1 nm/s and the total evaporation coating thickness was 60 nm;

the compound T1 was evaporated above the hole transport layer under vacuum as an electron blocking layer of the device, where the evaporation rate was 0.1 nm/s and the total evaporation coating thickness was 60 nm;

a luminescent layer of the device was evaporated above the electron blocking layer under vacuum; the luminescent layer includes a host material and a dyeing material; a multi-source co-evaporation method was used to adjust the evaporation rate of the host material GPH-59 to 0.1 nm/s; the evaporation rate of the dye RPD-8 was set by a ratio of 3%, and the total evaporation coating thickness was 40 nm;

an electron transport layer (material: ET-46) was evaporated above the luminescent layer under vacuum, and set by a ratio of 50%, and ET-57 was set by a ratio of 50%, where the evaporation rate was 0.1 nm/s and the total evaporation coating thickness was 25 nm;

LIF with a thickness of 0.5 nm, as an electron injection layer, was evaporated above the electron transport layer (ETL) under vacuum, and an Al layer with a thickness of 150 nm served as a cathode of the device.

### Examples 3-2 to 3-25 and Comparative Example 3-1

The preparing process of Examples 3-2 to 3-12 and Comparative Example 3-1 is the same with that in Example 3-1, and the difference is that the compound T1 of the electron blocking layer material is replaced with the compounds as shown in Table 3.

The electron blocking layer material in Comparative Example 3-1 has the following structure (see details in patent WO2019/004587A1)

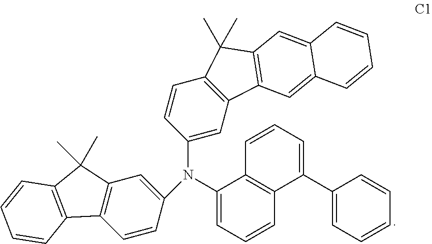 [SMILES: CC1(C)c2ccccc2-c2ccc(N(c3ccc4c(c3)-c3cc5ccccc5cc3C4(C)C)c3cccc4c(-c5ccccc5)cccc34)cc21]

Performance measurement is performed on the organic electroluminescent device prepared by the above process below:

at a same luminance value, a PR750 photoradiometer and an ST-86LA luminance meter (Beijing Normal University Photoelectric Instrument Plant) as well as a Keithley4200 test system were used to measure the driving voltage, current efficiency and service life of the organic electroluminescent device prepared in Examples and Comparative Examples. Specifically, voltage was increased at a rate of 0.1 V/s, when the luminance of the organic electroluminescent device was up to 5000 cd/m², the voltage was measured as the driving voltage, and electric current density was measured at this time simultaneously; the ratio of the luminance to the electric current density was the current efficiency; LT95 life test was as follows: a luminance meter was used to keep a constant current under a luminance value of 5000 cd/m²; the time when the luminance of the organic electroluminescent device dropped to 4750 cd/m² was measured with a unit of hour. The service life in Comparative Example 3-1 was set as a standard 100%, others were the ratios thereto. The measured results were shown in table 4.

TABLE 4

| | Electron | | | | |
|---|---|---|---|---|---|
| | blocking | Desired | | Current | LT95 |
| | layer | luminance | Voltage | efficiency | service |
| | material | cd/m | V | cd/A | life % |
| Comparative | C1 | 5000.00 | 5.5 | 13 | 100 |
| Example 3-1 | | | | | |
| Examples 3-1 | T1 | 5000.00 | 5.0 | 17.2 | 250 |
| Examples 3-2 | T2 | 5000.00 | 4.8 | 18.3 | 300 |
| Examples 3-3 | T11 | 5000.00 | 4.9 | 18.1 | 289 |
| Examples 3-4 | T12 | 5000.00 | 4.5 | 17.6 | 310 |
| Examples 3-5 | T81 | 5000.00 | 4.8 | 16.4 | 350 |
| Examples 3-6 | T163 | 5000.00 | 5.0 | 17.5 | 276 |
| Examples 3-7 | T170 | 5000.00 | 4.7 | 18.1 | 360 |
| Examples 3-8 | T232 | 5000.00 | 4.8 | 17.9 | 300 |
| Examples 3-9 | T54 | 5000.00 | 4.8 | 18.1 | 350 |
| Examples 3-10 | T237 | 5000.00 | 5.2 | 17.2 | 290 |
| Examples 3-11 | T248 | 5000.00 | 4.8 | 17.6 | 320 |
| Examples 3-12 | T255 | 5000.00 | 4.9 | 17.8 | 330 |

It can be seen from the results of Table 4 that when the compounds provided by the present invention are used as the electron blocking layer materials of the organic electroluminescent device, and when the luminance is up to 5000 cd/m², the driving voltage is 4.5-5.2V, and the current efficiency is 16.4-18.3 cd/A. Therefore, the compounds of the present invention may effectively reduce the driving voltage, improve the current efficiency and prolong the service life of the device, and thus is a kind of electron blocking material with good performances.

In the electron blocking layer material C1 used in Comparative Example 1-1, the group substituted on the naphthalene ring is phenyl, and there is no binaphthyl group in the present invention. Therefore, the performance of the device in Comparative Example 1-1 decreases obviously relative to the examples, and the driving voltage is up to 5.5 V, while the current efficiency is only 13 cd/A.

Apparently, the above examples are merely used to specify the present invention clearly, but are not intended for limiting the embodiments. A person skilled in the art may make other changes or alterations in different forms based on the above description. All the embodiments need not be and may not be illustrated herein. Apparent changes or alterations derived thereby should fall within the protection scope of the present invention.

What is claimed is:

1. A compound having a structure as shown in Formula (I): 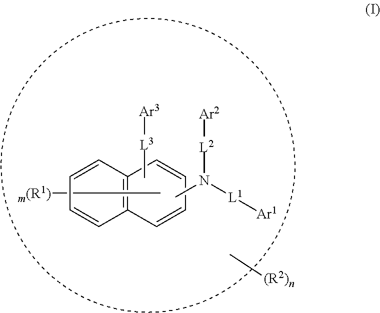
in the Formula (I), Ar¹ and Ar² are each independently selected from H, a substituted or unsubstituted C₆-C₅₀ aryl, a substituted or unsubstituted C₃-C₃₀ heteroaryl, a substituted or unsubstituted C₆-C₅₀ fused aryl, a substituted or unsubstituted C₃-C₃₀ fused heteroaryl; and when Ar¹ is H, L¹ is not a single bond; when Ar² is H, L² is not a single bond; Ar³ is selected from a substituted or unsubstituted C₆-C₅₀ aryl, a substituted or unsubstituted C₃-C₃₀ heteroaryl, a substituted or unsubstituted C₆-C₅₀ fused aryl, and a substituted or unsubstituted C₃-C₃₀ fused heteroaryl;
L¹-L³ are each independently selected from a single bond, a substituted or unsubstituted C₁-C₁₀ alkylene, a substituted or unsubstituted C₆-C₅₀ arylene, and a substituted or unsubstituted C₃-C₃₀ heteroarylene group;
m is an integer of 0-6, and n is an integer of 0-15;
R¹ is each independently selected from H, a halogen, carbonyl, carboxyl, amino, amido, cyano, nitryl, an ester group, hydroxyl, silicyl, a substituted or unsubstituted C₁-C₂₀ alkyl, a substituted or unsubstituted C₃-C₂₀ cycloalkyl, a substituted or unsubstituted C₂-C₂₀ alkenyl, a substituted or unsubstituted C₂-C₂₀ alkynyl, a substituted or unsubstituted C₁-C₂₀ alkoxy, a substituted or unsubstituted C₃-C₁₀ cycloalkoxy, a substituted or unsubstituted C₆-C₅₀ aryl, a substituted or unsubstituted C₃-C₃₀ heteroaryl, and a C₆-C₅₀ fused aryl;
R² is, on each occurrence, a substituent of Ar¹-Ar³, L¹-L³, R¹ or the naphthalene ring in the Formula (I), and is each independently selected from H, a halogen, carbonyl, carboxyl, amino, amido, cyano, nitryl, an ester group, hydroxyl, a C₁-C₁₀ silicyl, a substituted or unsubstituted C₁-C₂₀ alkyl, a substituted or unsubstituted C₃-C₂₀ cycloalkyl, a substituted or unsubstituted C₂-C₁₂ alkenyl, a substituted or unsubstituted C₂-C₁₂ alkynyl, a substituted or unsubstituted C₁-C₁₂ alkoxy, a substituted or unsubstituted C₃-C₁₀ cycloalkoxy, a substituted or unsubstituted C₆-C₅₀ aryl, a substituted or unsubstituted C₃-C₃₀ heteroaryl, and a C₆-C₅₀ fused aryl;
the group 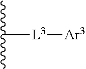
is located in an ortho position of the group 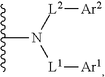
and neither R¹ nor R² is amido; or Ar¹ is a substituted or unsubstituted C₆-C₃₀ aryl or a substituted or unsubstituted C₃-C₃₀ heteroaryl, Ar² is a substituted or unsubstituted benzodimethyl fluorenyl, and Ar³ is a substituted or unsubstituted naphthyl;
when each substituted or unsubstituted group has a substituent, the substituent is selected from one or more of a halogen, cyano, nitryl, an ester group, hydroxyl, carbonyl, carboxyl, cyano, amido, a C₁-C₁₀ silicyl, a C₁-C₂₀ alkyl, a C₃-C₂₀ cycloalkyl, a C₂-C₂₀ alkenyl, a C₂-C₁₀ alkynyl, a C₁-C₂₀ alkoxy or thioalkoxy, a C₆-C₃₀ arylamino, a C₃-C₃₀ heteroarylamino, a C₆-C₃₀ monocyclic or fused-cyclic aryl, a C₃-C₃₀ monocyclic or fused-cyclic heteroaryl.

2. The compound according to claim 1, wherein
the group 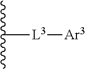
is located in an ortho position of the group 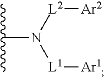
Ar¹-Ar³ are each independently selected from a substituted or unsubstituted C₆-C₃₀ aryl or a substituted or unsubstituted C₃-C₃₀ heteroaryl,
L¹-L³ are each independently selected from a single bond, a substituted or unsubstituted C₆-C₃₀ alkylene, and a substituted or unsubstituted C₆-C₃₀ heteroarylene group;
m is an integer of 1-6, and n is an integer of 1-15;
R¹ is each independently selected from one of H, a halogen, cyano, nitryl, hydroxyl, silicyl, a C₁-C₂₀ chain-typed alkyl, a C₃-C₂₀ cycloalkyl, a C₂-C₂₀ alkenyl, a C₂-C₂₀ alkynyl, a C₁-C₂₀ alkoxy, a substituted or unsubstituted C₆-C₃₀ aryl, and a substituted or unsubstituted C₁-C₃₀ heteroaryl;
R² is, on each occurrence, a substituent of Ar¹-Ar³, L¹-L³, R¹ or the naphthalene ring in the Formula (I), and is each independently selected from one of H, a substituted or unsubstituted C₃-C₃₀ cycloalkyl; and at least one R² is selected from one of the substituted or unsubstituted C₃-C₃₀ cycloalkyl;
when each substituted or unsubstituted group has a substituent, the substituent is selected from one or a combination of more of a halogen, a C₁-C₂₀ chain-typed alkyl, a C₃-C₂₀ cycloalkyl, a C₂-C₂₀ alkenyl, a C₁-C₂₀ alkoxy or thioalkoxy, a C₆-C₃₀ monocyclic or fused-cyclic aryl, a C₃-C₃₀ monocyclic or fused-cyclic heteroaryl.

3. The compound according to claim 2, wherein
L¹ and L² are each independently selected from a single bond, phenylene or naphthylene, and L³ is a single bond; Ar¹ is a substituted or unsubstituted C₁₀-C₃₀ fused-cyclic aryl or a substituted or unsubstituted C₆-C₃₀ fused-cyclic heteroaryl; Ar² is a substituted or unsubstituted C₆-C₃₀ monocyclic aryl or a substituted or unsubstituted C₃-C₃₀ heteroaryl; Ar³ is a substituted or unsubstituted naphthyl, a substituted or unsubstituted fluorenyl, or a substituted or unsubstituted dibenzo-X hetercyclopentadiene, X is O, N, S, or Si; when each substituted or unsubstituted group has a substituent, the substituent is selected from a C₁-C₂₀ chain-typed alkyl, a C₃-C₂₀ cycloalkyl, a C₆-C₃₀ aryl or a C₃-C₃₀ cycloalkyl;
R² is selected from one of the following structures: 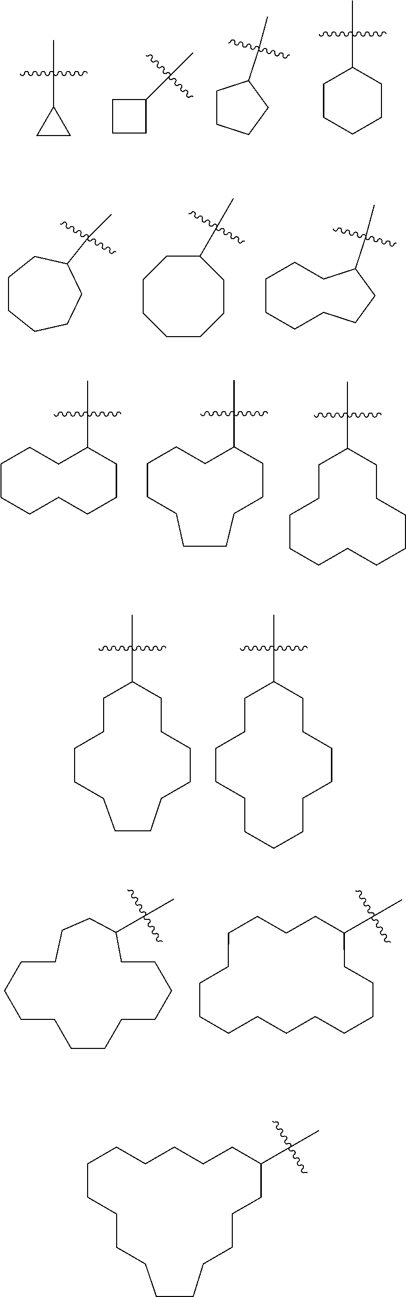 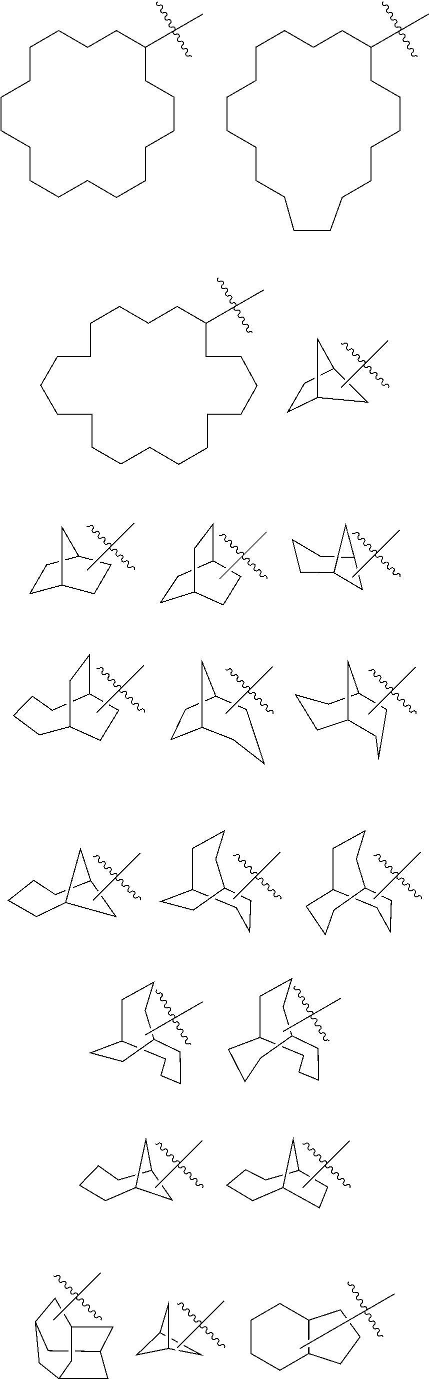 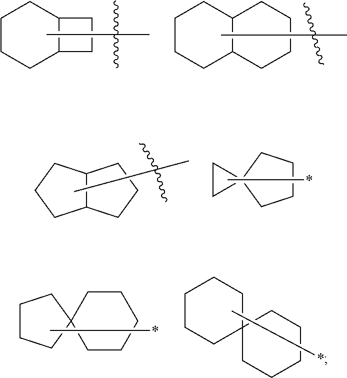
preferably, Ar¹ is selected from one of the following structures: 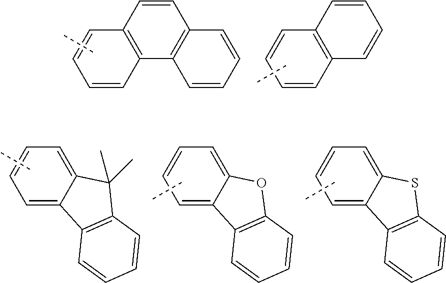
Ar² is selected from one of the following structures: 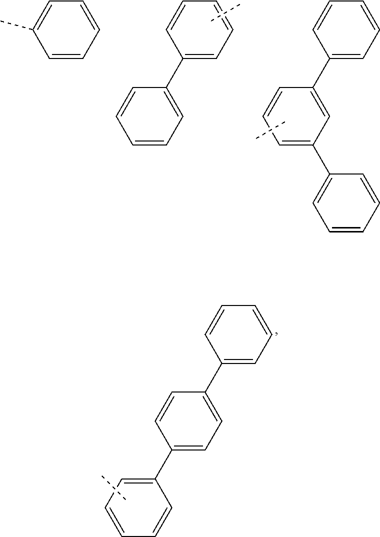
wherein, the dotted line denotes an access site of a group; the representing method of lining across the benzene ring with the dotted line denotes that a linking site of a group may be in any bondable position on the benzene ring;
preferably, the group 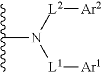
is located in a 1-position or 2-position on the naphthalene ring, and when the group 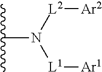
is located in the 1-position on the naphthalene ring, the group 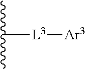
is located in the 2-position on the naphthalene ring;
preferably, R² is each independently selected from cyclopentyl, cyclohexyl and cycloheptyl;
more preferably, at least one of Ar¹ and Ar² has a substituent of substituted or unsubstituted C₃-C₂₀ cycloalkyl;
further preferably, Ar² has the substituent of substituted or unsubstituted C₃-C₂₀ cycloalkyl.

4. The compound according to claim 2, wherein the compound has a structure as shown in P1-P291:

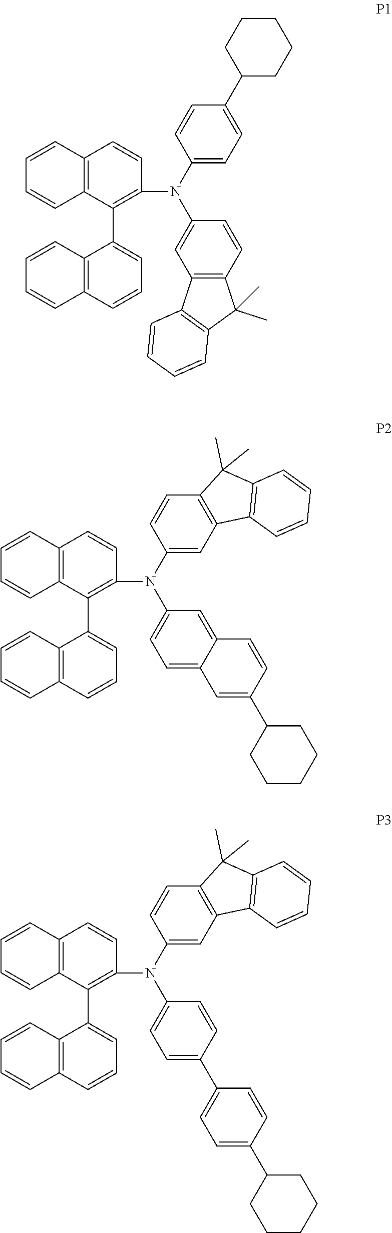 [SMILES: CC1(C)c2ccccc2-c2cc(N(c3ccc(C4CCCCC4)cc3)c3ccc4ccccc4c3-c3cccc4ccccc34)ccc21 ; CC1(C)c2ccccc2-c2cc(N(c3ccc4cc(C5CCCCC5)ccc4c3)c3ccc4ccccc4c3-c3cccc4ccccc34)ccc21 ; CC1(C)c2ccccc2-c2cc(N(c3ccc(-c4ccc(C5CCCCC5)cc4)cc3)c3ccc4ccccc4c3-c3cccc4ccccc34)ccc21]

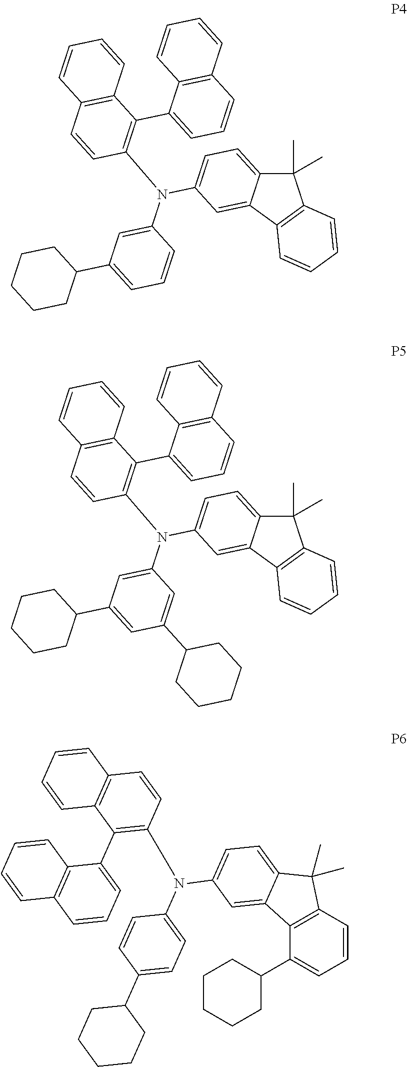 [SMILES: CC1(C)c2ccccc2-c2cc(N(c3cccc(C4CCCCC4)c3)c3ccc4ccccc4c3-c3cccc4ccccc34)ccc21 ; CC1(C)c2ccccc2-c2cc(N(c3cc(C4CCCCC4)cc(C4CCCCC4)c3)c3ccc4ccccc4c3-c3cccc4ccccc34)ccc21 ; CC1(C)c2ccc(N(c3ccc(C4CCCCC4)cc3)c3ccc4ccccc4c3-c3cccc4ccccc34)cc2-c2c(C3CCCCC3)cccc21]

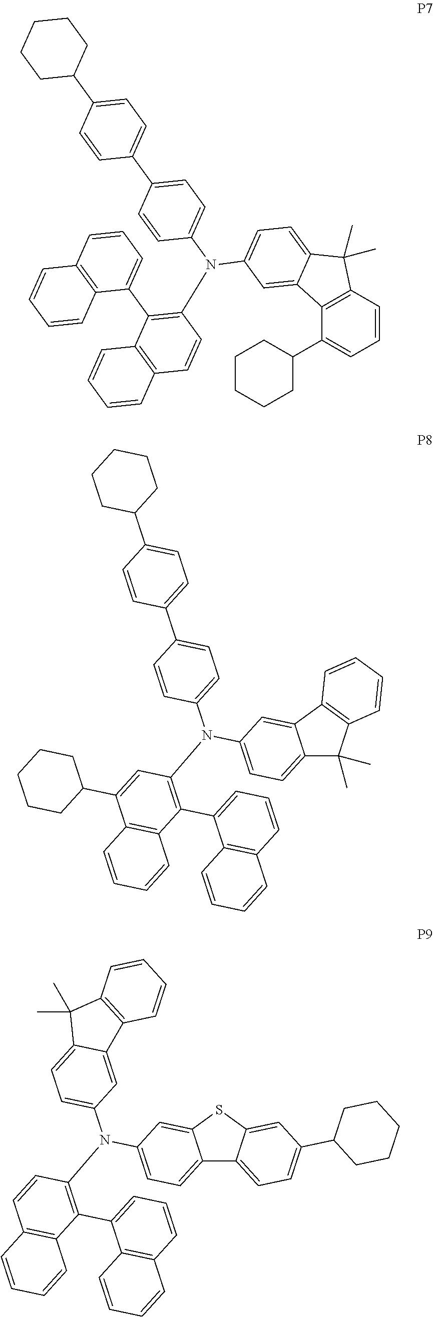

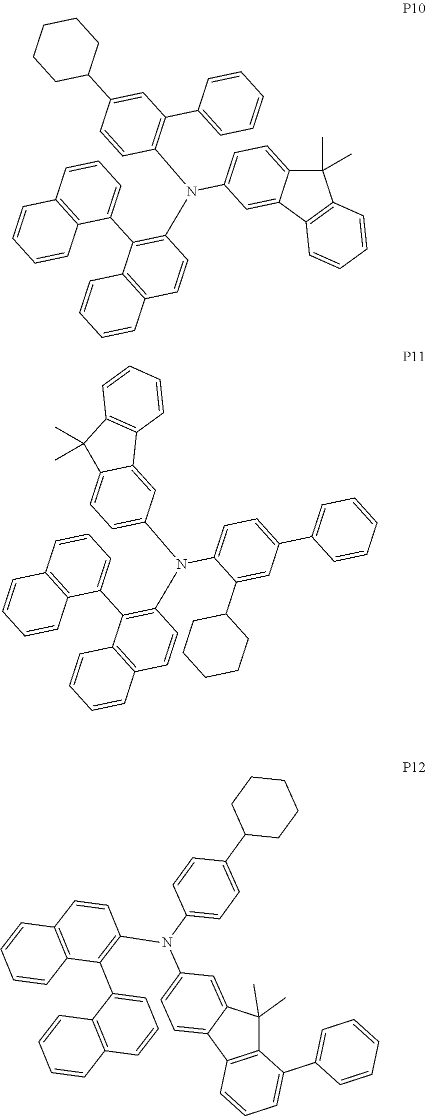

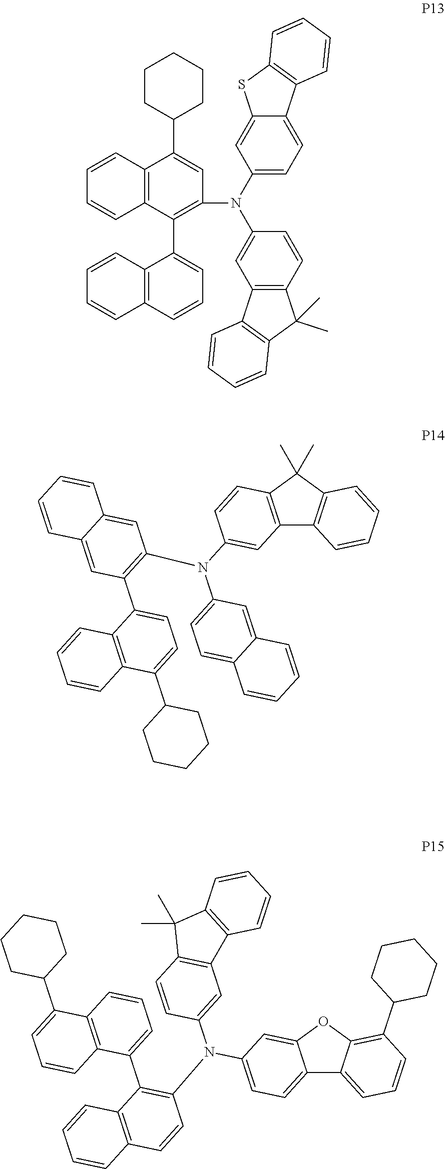 [SMILES: CC1(C)c2ccccc2-c2cc(N(c3ccc4c(c3)sc3ccccc34)c3cc(C4CCCCC4)c4ccccc4c3-c3cccc4ccccc34)ccc21 ; CC1(C)c2ccccc2-c2cc(N(c3ccc4ccccc4c3)c3cc4ccccc4cc3-c3ccc(C4CCCCC4)c4ccccc34)ccc21 ; CC1(C)c2ccccc2-c2cc(N(c3ccc4c(c3)oc3c(C5CCCCC5)cccc34)c3ccc4ccccc4c3-c3cccc4c(C5CCCCC5)cccc34)ccc21]

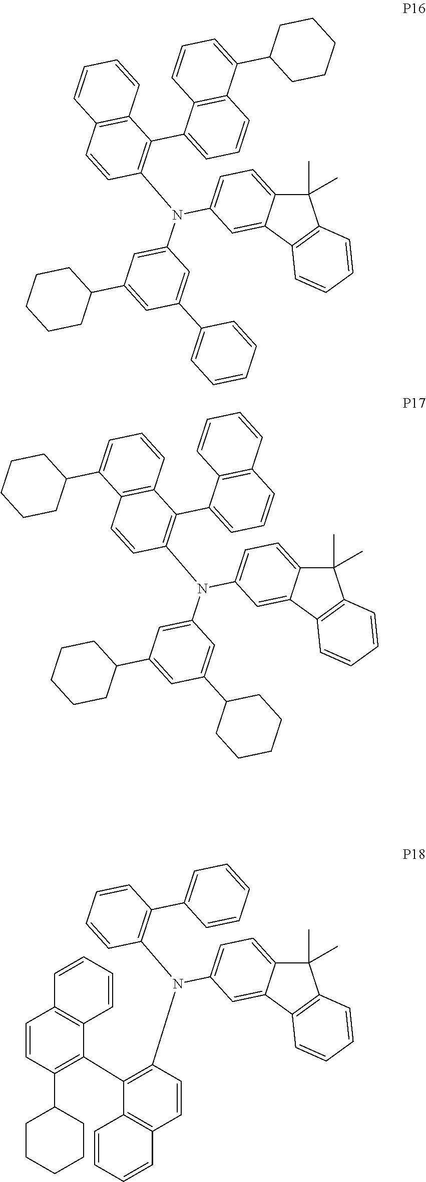

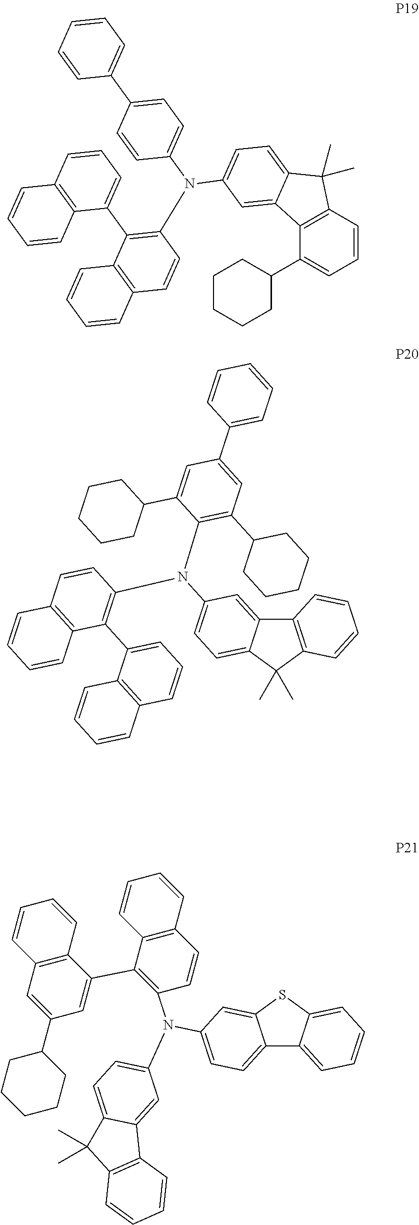

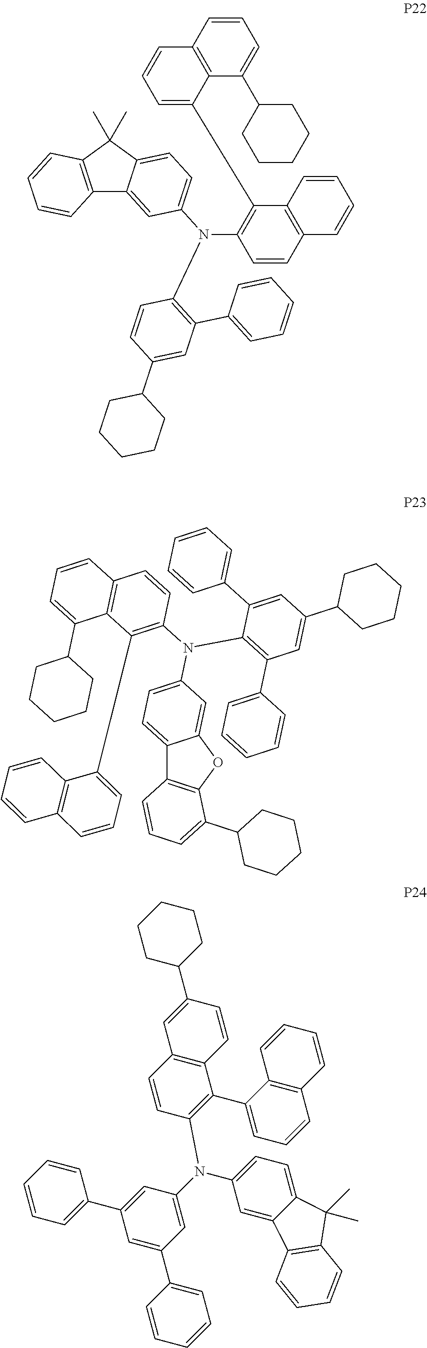 [SMILES: CC1(C)c2ccccc2-c2cc(N(c3ccc(C4CCCCC4)cc3-c3ccccc3)c3ccc4ccccc4c3-c3cccc4cccc(C5CCCCC5)c34)ccc21 ; c1ccc(-c2cc(C3CCCCC3)cc(-c3ccccc3)c2N(c2ccc3c(c2)oc2c(C4CCCCC4)cccc23)c2ccc3cccc(C4CCCCC4)c3c2-c2cccc3ccccc23)cc1 ; CC1(C)c2ccccc2-c2cc(N(c3cc(-c4ccccc4)cc(-c4ccccc4)c3)c3ccc4cc(C5CCCCC5)ccc4c3-c3cccc4ccccc34)ccc21]

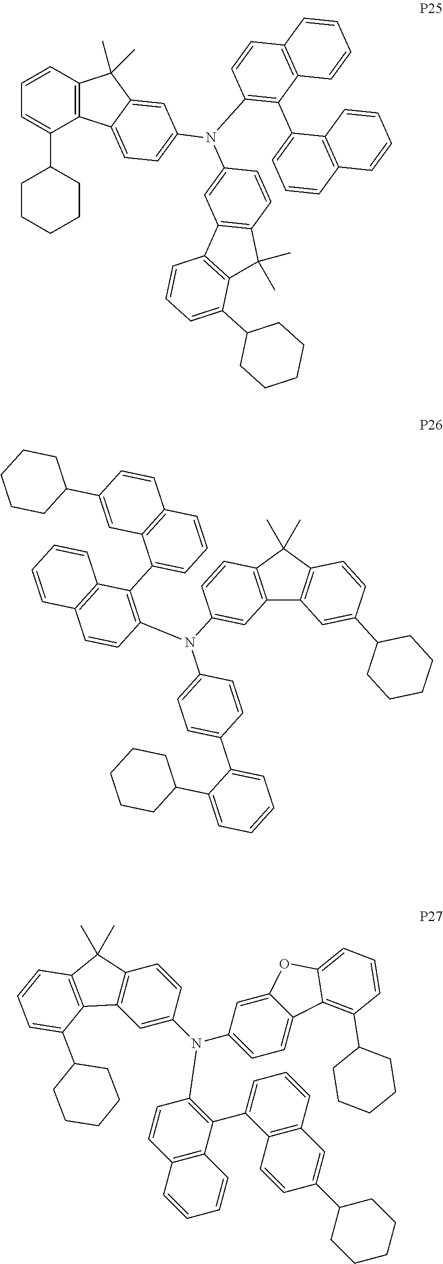 [SMILES: CC1(C)c2cc(N(c3ccc4c(c3)-c3cccc(C5CCCCC5)c3C4(C)C)c3ccc4ccccc4c3-c3cccc4ccccc34)ccc2-c2c(C3CCCCC3)cccc21 ; CC1(C)c2ccc(C3CCCCC3)cc2-c2cc(N(c3ccc(-c4ccccc4C4CCCCC4)cc3)c3ccc4ccccc4c3-c3cccc4ccc(C5CCCCC5)cc34)ccc21 ; CC1(C)c2ccc(N(c3ccc4c(c3)oc3cccc(C5CCCCC5)c34)c3ccc4ccccc4c3-c3cccc4cc(C5CCCCC5)ccc34)cc2-c2c(C3CCCCC3)cccc21]

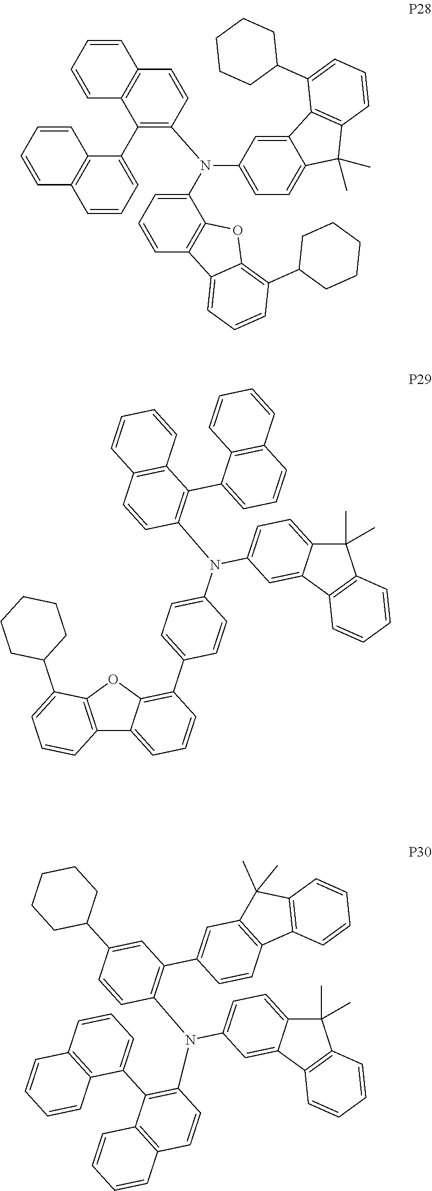 [SMILES: CC1(C)c2ccc(N(c3ccc4ccccc4c3-c3cccc4ccccc34)c3cccc4c3oc3c(C5CCCCC5)cccc34)cc2-c2c(C3CCCCC3)cccc21 ; CC1(C)c2ccccc2-c2cc(N(c3ccc(-c4cccc5c4oc4c(C6CCCCC6)cccc45)cc3)c3ccc4ccccc4c3-c3cccc4ccccc34)ccc21 ; CC1(C)c2ccccc2-c2cc(N(c3ccc(C4CCCCC4)cc3-c3ccc4c(c3)C(C)(C)c3ccccc3-4)c3ccc4ccccc4c3-c3cccc4ccccc34)ccc21]

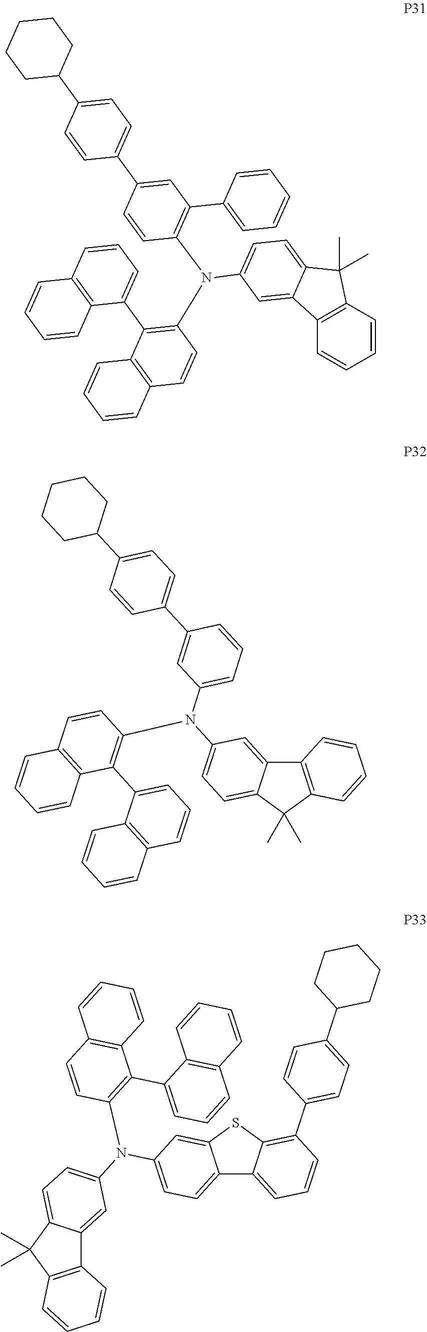 [SMILES: CC1(C)c2ccccc2-c2cc(N(c3ccc(-c4ccc(C5CCCCC5)cc4)cc3-c3ccccc3)c3ccc4ccccc4c3-c3cccc4ccccc34)ccc21 ; CC1(C)c2ccccc2-c2cc(N(c3cccc(-c4ccc(C5CCCCC5)cc4)c3)c3ccc4ccccc4c3-c3cccc4ccccc34)ccc21 ; CC1(C)c2ccccc2-c2cc(N(c3ccc4c(c3)sc3c(-c5ccc(C6CCCCC6)cc5)cccc34)c3ccc4ccccc4c3-c3cccc4ccccc34)ccc21]

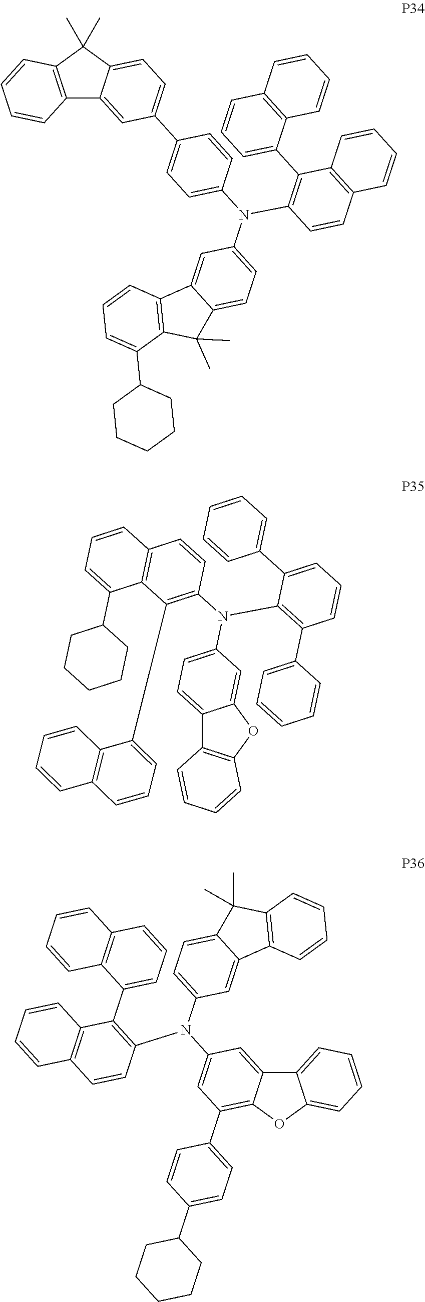 [SMILES: CC1(C)c2ccccc2-c2cc(-c3ccc(N(c4ccc5c(c4)-c4cccc(C6CCCCC6)c4C5(C)C)c4ccc5ccccc5c4-c4cccc5ccccc45)cc3)ccc21 ; c1ccc(-c2cccc(-c3ccccc3)c2N(c2ccc3c(c2)oc2ccccc23)c2ccc3cccc(C4CCCCC4)c3c2-c2cccc3ccccc23)cc1 ; CC1(C)c2ccccc2-c2cc(N(c3cc(-c4ccc(C5CCCCC5)cc4)c4oc5ccccc5c4c3)c3ccc4ccccc4c3-c3cccc4ccccc34)ccc21]

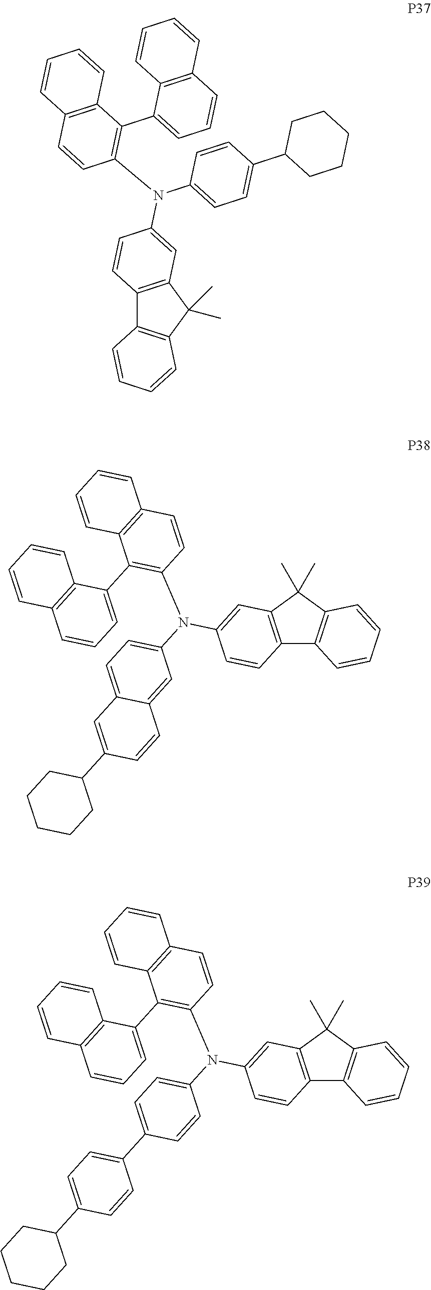 [SMILES: CC1(C)c2ccccc2-c2ccc(N(c3ccc(C4CCCCC4)cc3)c3ccc4ccccc4c3-c3cccc4ccccc34)cc21 ; CC1(C)c2ccccc2-c2ccc(N(c3ccc4cc(C5CCCCC5)ccc4c3)c3ccc4ccccc4c3-c3cccc4ccccc34)cc21 ; CC1(C)c2ccccc2-c2ccc(N(c3ccc(-c4ccc(C5CCCCC5)cc4)cc3)c3ccc4ccccc4c3-c3cccc4ccccc34)cc21]

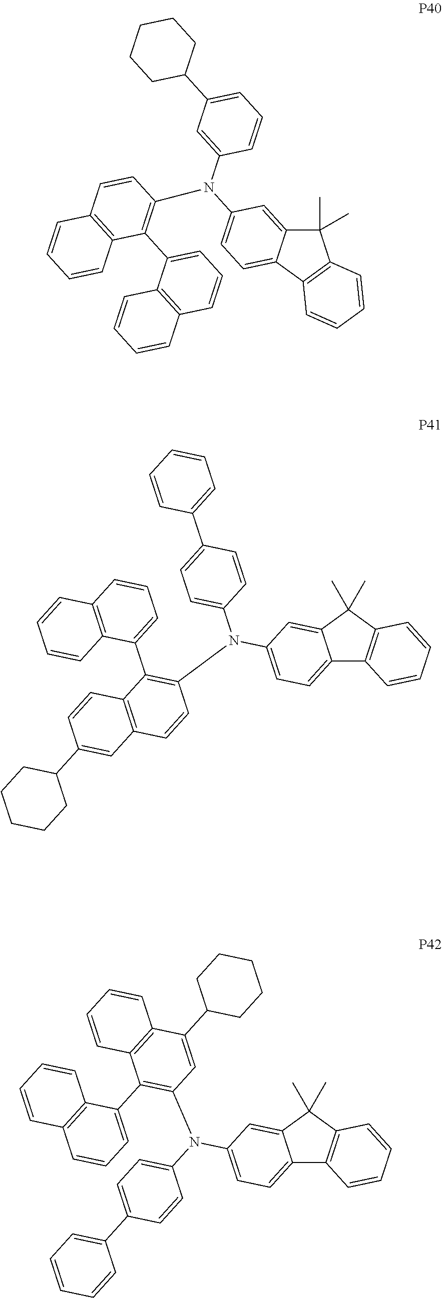 [SMILES: CC1(C)c2ccccc2-c2ccc(N(c3cccc(C4CCCCC4)c3)c3ccc4ccccc4c3-c3cccc4ccccc34)cc21 ; CC1(C)c2ccccc2-c2ccc(N(c3ccc(-c4ccccc4)cc3)c3ccc4cc(C5CCCCC5)ccc4c3-c3cccc4ccccc34)cc21 ; CC1(C)c2ccccc2-c2ccc(N(c3ccc(-c4ccccc4)cc3)c3cc(C4CCCCC4)c4ccccc4c3-c3cccc4ccccc34)cc21]

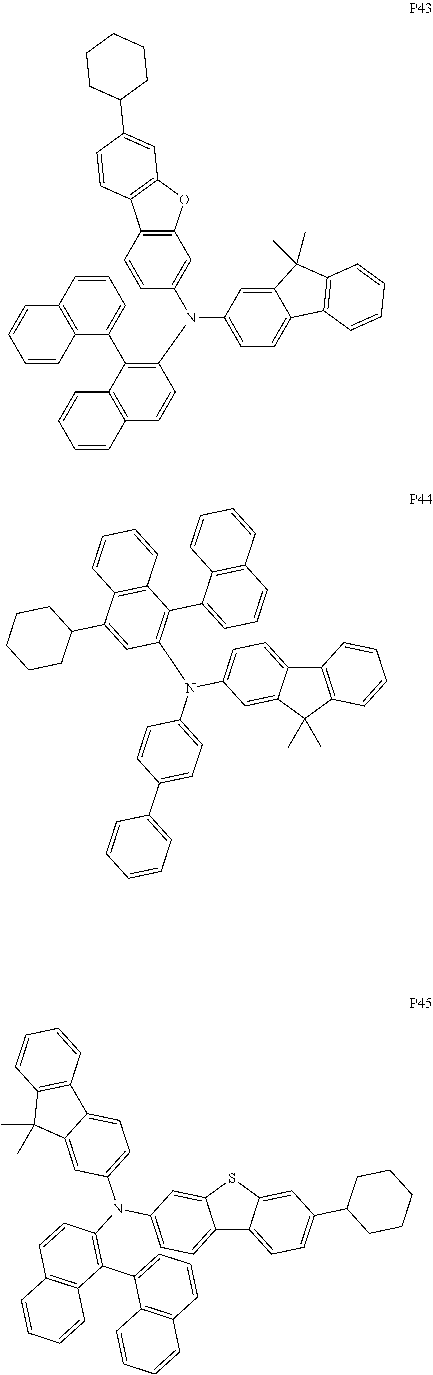 [SMILES: CC1(C)c2ccccc2-c2ccc(N(c3ccc4c(c3)oc3cc(C5CCCCC5)ccc34)c3ccc4ccccc4c3-c3cccc4ccccc34)cc21 ; CC1(C)c2ccccc2-c2ccc(N(c3ccc(-c4ccccc4)cc3)c3cc(C4CCCCC4)c4ccccc4c3-c3cccc4ccccc34)cc21 ; CC1(C)c2ccccc2-c2ccc(N(c3ccc4c(c3)sc3cc(C5CCCCC5)ccc34)c3ccc4ccccc4c3-c3cccc4ccccc34)cc21]

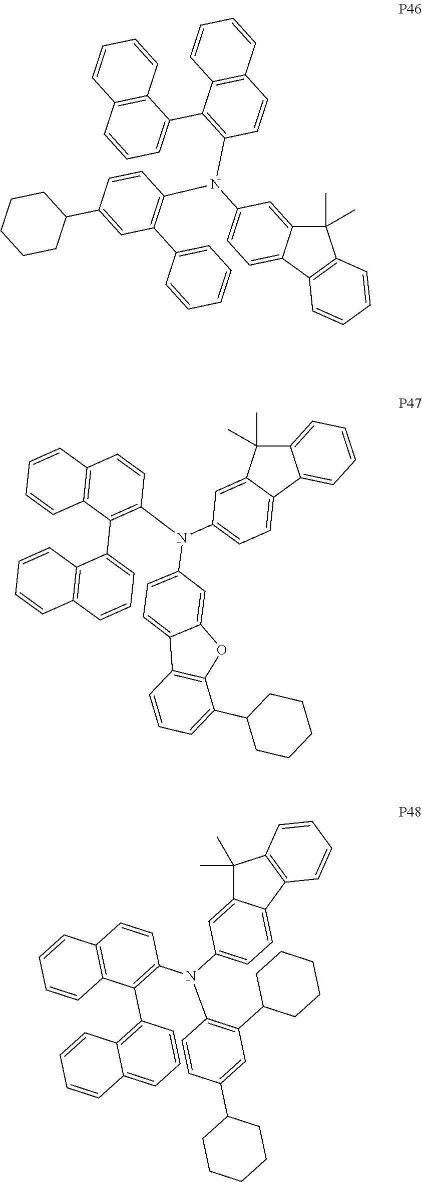

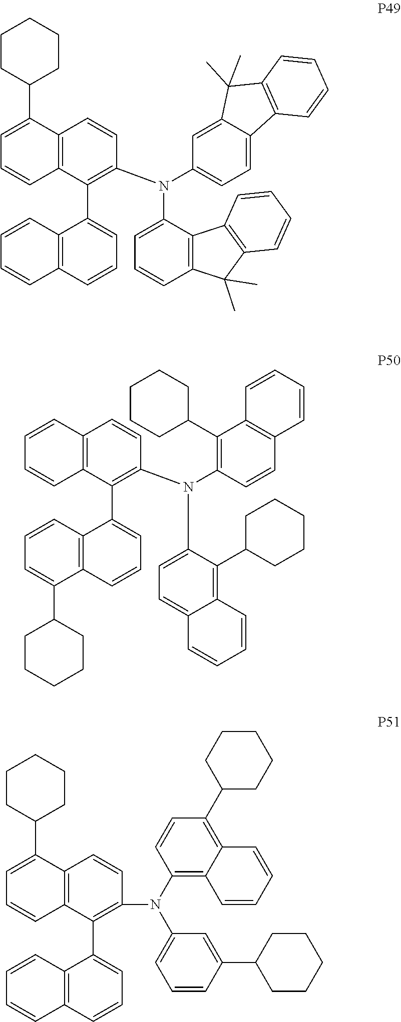 [SMILES: CC1(C)c2ccccc2-c2ccc(N(c3cccc4c3-c3ccccc3C4(C)C)c3ccc4c(C5CCCCC5)cccc4c3-c3cccc4ccccc34)cc21 ; c1ccc2c(-c3cccc4c(C5CCCCC5)cccc34)c(N(c3ccc4ccccc4c3C3CCCCC3)c3ccc4ccccc4c3C3CCCCC3)ccc2c1 ; c1cc(C2CCCCC2)cc(N(c2ccc3c(C4CCCCC4)cccc3c2-c2cccc3ccccc23)c2ccc(C3CCCCC3)c3ccccc23)c1]

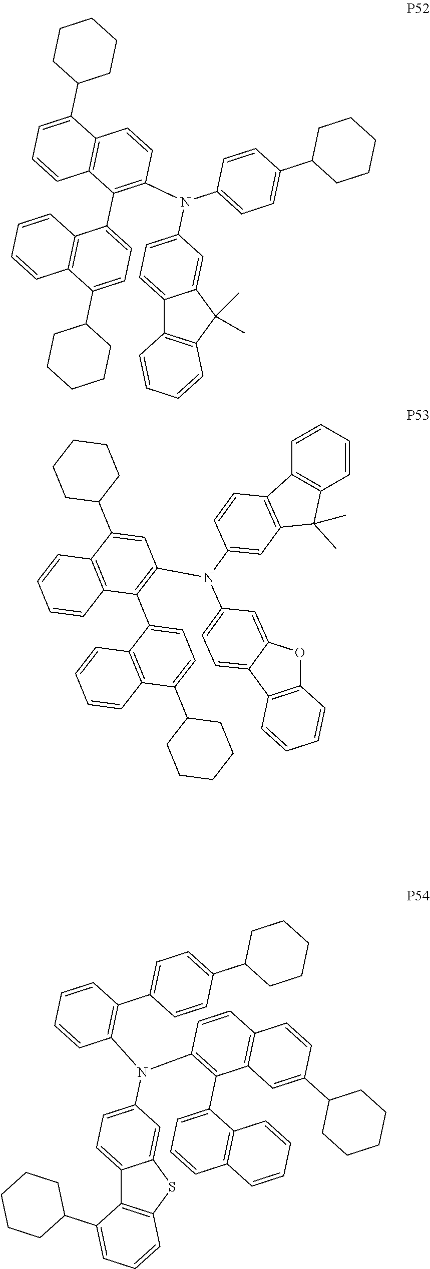 [SMILES: CC1(C)c2ccccc2-c2ccc(N(c3ccc(C4CCCCC4)cc3)c3ccc4c(C5CCCCC5)cccc4c3-c3ccc(C4CCCCC4)c4ccccc34)cc21 ; CC1(C)c2ccccc2-c2ccc(N(c3ccc4c(c3)oc3ccccc34)c3cc(C4CCCCC4)c4ccccc4c3-c3ccc(C4CCCCC4)c4ccccc34)cc21 ; c1ccc(N(c2ccc3c(c2)sc2cccc(C4CCCCC4)c23)c2ccc3ccc(C4CCCCC4)cc3c2-c2cccc3ccccc23)c(-c2ccc(C3CCCCC3)cc2)c1]

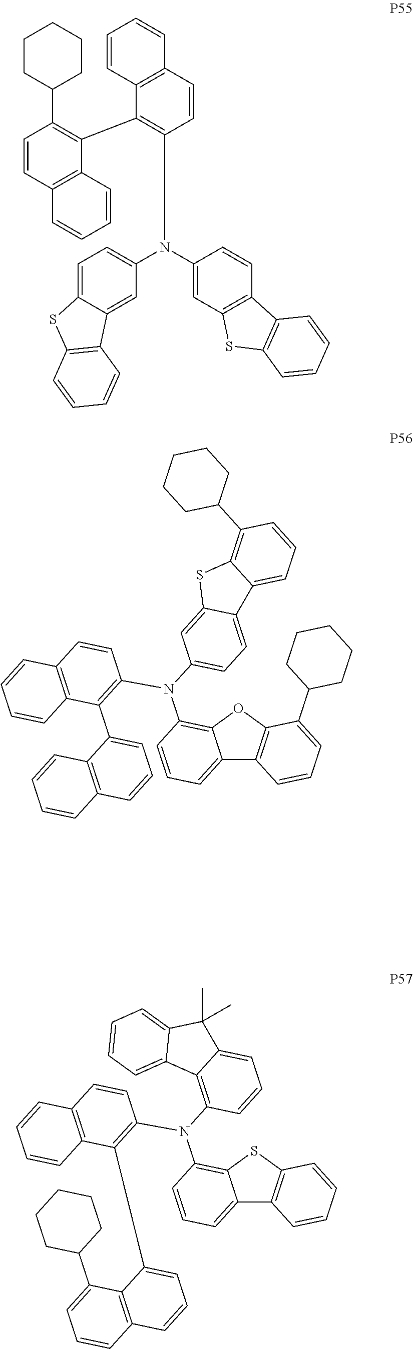 [SMILES: c1ccc2c(-c3c(N(c4ccc5c(c4)sc4ccccc45)c4ccc5sc6ccccc6c5c4)ccc4ccccc34)c(C3CCCCC3)ccc2c1 ; c1ccc2c(-c3c(N(c4ccc5c(c4)sc4c(C6CCCCC6)cccc45)c4cccc5c4oc4c(C6CCCCC6)cccc45)ccc4ccccc34)cccc2c1 ; CC1(C)c2ccccc2-c2c(N(c3ccc4ccccc4c3-c3cccc4cccc(C5CCCCC5)c34)c3cccc4c3sc3ccccc34)cccc21]

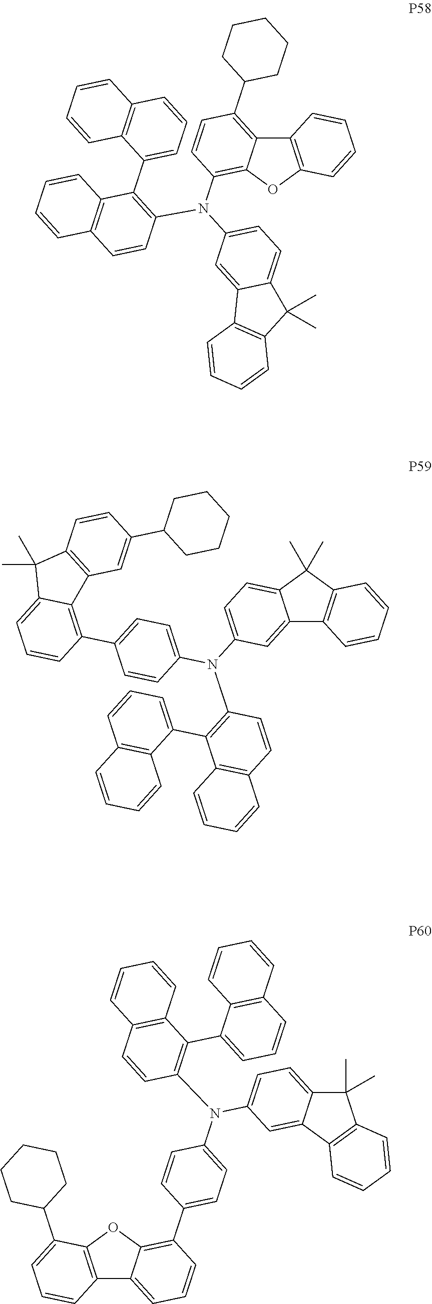 [SMILES: CC1(C)c2ccccc2-c2cc(N(c3ccc4ccccc4c3-c3cccc4ccccc34)c3ccc(C4CCCCC4)c4c3oc3ccccc34)ccc21 ; CC1(C)c2ccccc2-c2cc(N(c3ccc(-c4cccc5c4-c4cc(C6CCCCC6)ccc4C5(C)C)cc3)c3ccc4ccccc4c3-c3cccc4ccccc34)ccc21 ; CC1(C)c2ccccc2-c2cc(N(c3ccc(-c4cccc5c4oc4c(C6CCCCC6)cccc45)cc3)c3ccc4ccccc4c3-c3cccc4ccccc34)ccc21]

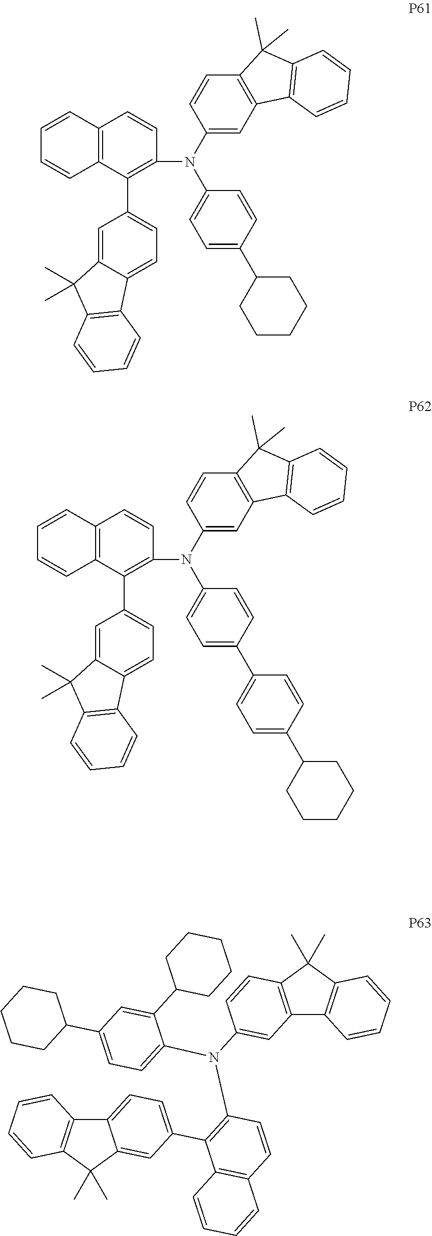 [SMILES: CC1(C)c2ccccc2-c2cc(N(c3ccc(C4CCCCC4)cc3)c3ccc4ccccc4c3-c3ccc4c(c3)C(C)(C)c3ccccc3-4)ccc21 ; CC1(C)c2ccccc2-c2cc(N(c3ccc(-c4ccc(C5CCCCC5)cc4)cc3)c3ccc4ccccc4c3-c3ccc4c(c3)C(C)(C)c3ccccc3-4)ccc21 ; CC1(C)c2ccccc2-c2cc(N(c3ccc(C4CCCCC4)cc3C3CCCCC3)c3ccc4ccccc4c3-c3ccc4c(c3)C(C)(C)c3ccccc3-4)ccc21]

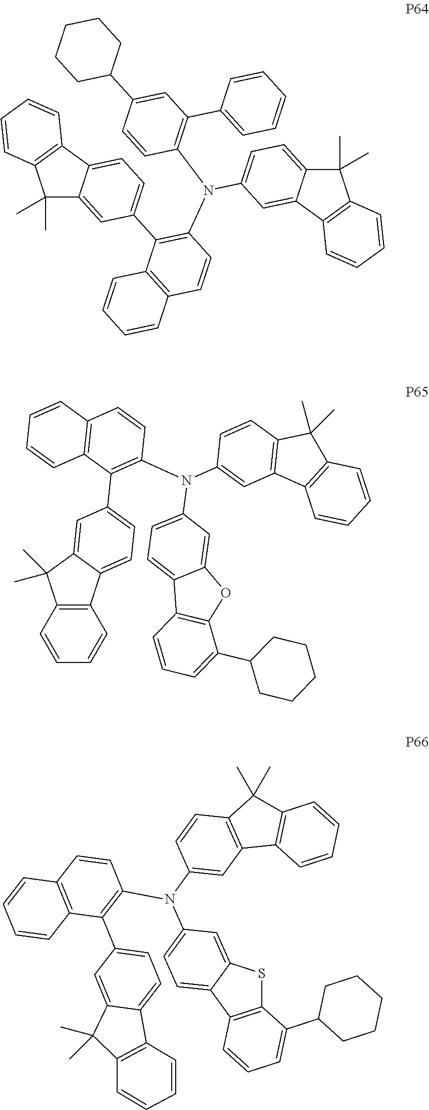 [SMILES: CC1(C)c2ccccc2-c2cc(N(c3ccc(C4CCCCC4)cc3-c3ccccc3)c3ccc4ccccc4c3-c3ccc4c(c3)C(C)(C)c3ccccc3-4)ccc21 ; CC1(C)c2ccccc2-c2cc(N(c3ccc4c(c3)oc3c(C5CCCCC5)cccc34)c3ccc4ccccc4c3-c3ccc4c(c3)C(C)(C)c3ccccc3-4)ccc21 ; CC1(C)c2ccccc2-c2cc(N(c3ccc4c(c3)sc3c(C5CCCCC5)cccc34)c3ccc4ccccc4c3-c3ccc4c(c3)C(C)(C)c3ccccc3-4)ccc21]

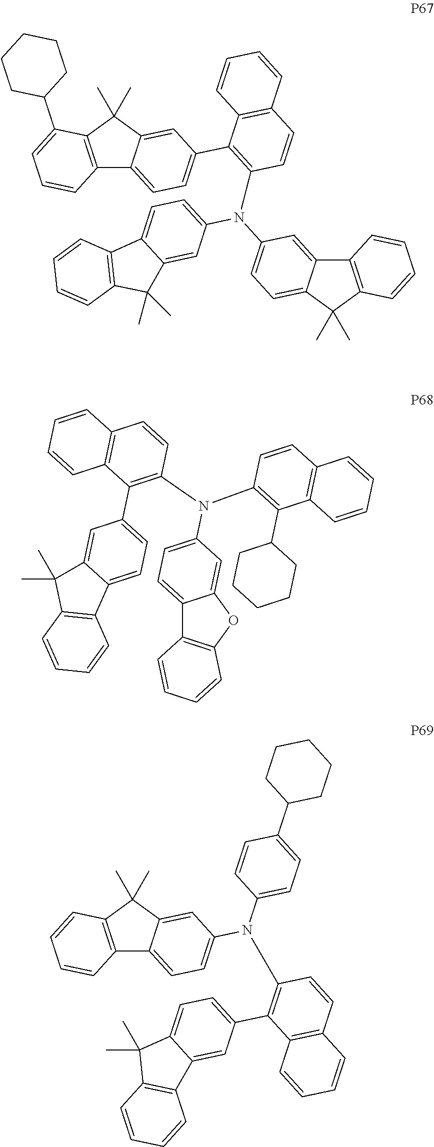 [SMILES: CC1(C)c2ccccc2-c2cc(N(c3ccc4c(c3)C(C)(C)c3ccccc3-4)c3ccc4ccccc4c3-c3ccc4c(c3)C(C)(C)c3c-4cccc3C3CCCCC3)ccc21 ; CC1(C)c2ccccc2-c2ccc(-c3c(N(c4ccc5c(c4)oc4ccccc45)c4ccc5ccccc5c4C4CCCCC4)ccc4ccccc34)cc21 ; CC1(C)c2ccccc2-c2cc(-c3c(N(c4ccc(C5CCCCC5)cc4)c4ccc5c(c4)C(C)(C)c4ccccc4-5)ccc4ccccc34)ccc21]

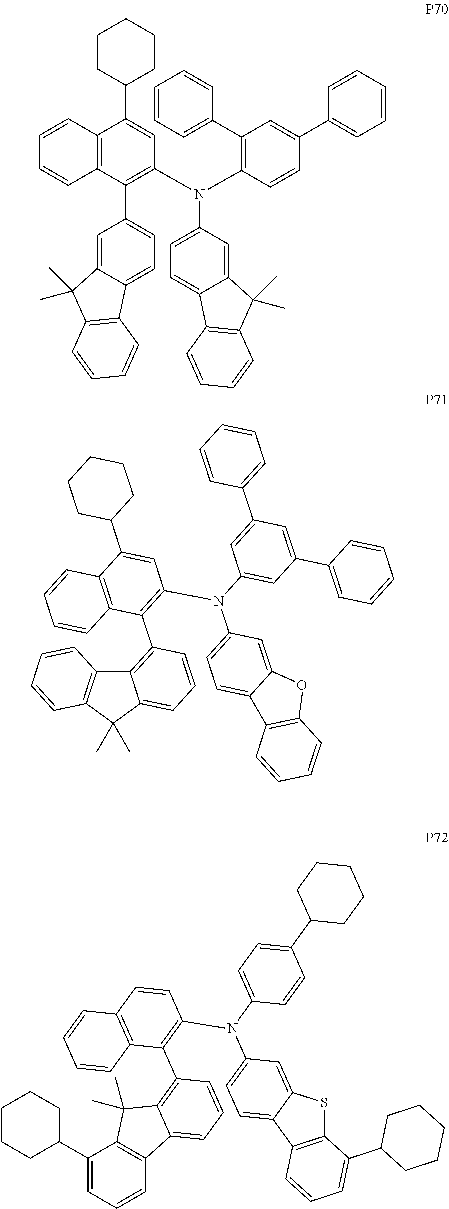

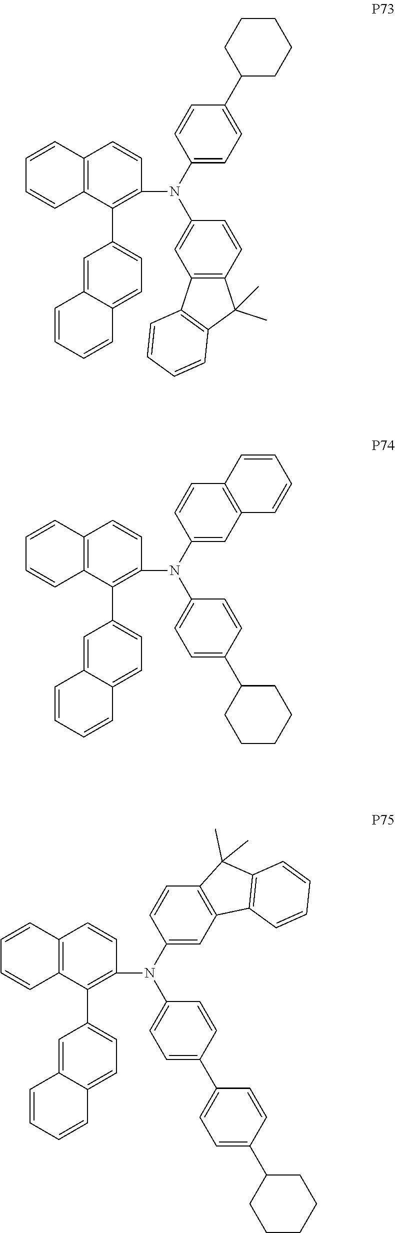 [SMILES: CC1(C)c2ccccc2-c2cc(N(c3ccc(C4CCCCC4)cc3)c3ccc4ccccc4c3-c3ccc4ccccc4c3)ccc21 ; c1ccc2cc(-c3c(N(c4ccc(C5CCCCC5)cc4)c4ccc5ccccc5c4)ccc4ccccc34)ccc2c1 ; CC1(C)c2ccccc2-c2cc(N(c3ccc(-c4ccc(C5CCCCC5)cc4)cc3)c3ccc4ccccc4c3-c3ccc4ccccc4c3)ccc21]

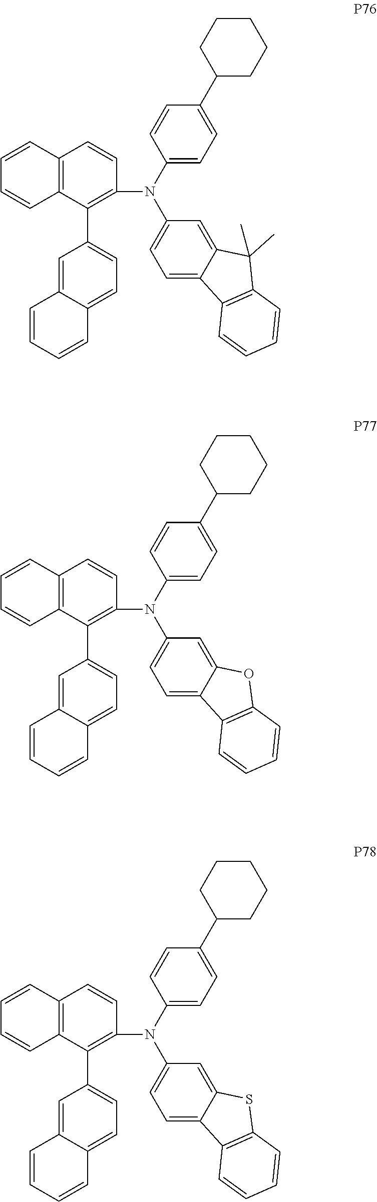 [SMILES: CC1(C)c2ccccc2-c2ccc(N(c3ccc(C4CCCCC4)cc3)c3ccc4ccccc4c3-c3ccc4ccccc4c3)cc21 ; c1ccc2cc(-c3c(N(c4ccc(C5CCCCC5)cc4)c4ccc5c(c4)oc4ccccc45)ccc4ccccc34)ccc2c1 ; c1ccc2cc(-c3c(N(c4ccc(C5CCCCC5)cc4)c4ccc5c(c4)sc4ccccc45)ccc4ccccc34)ccc2c1]

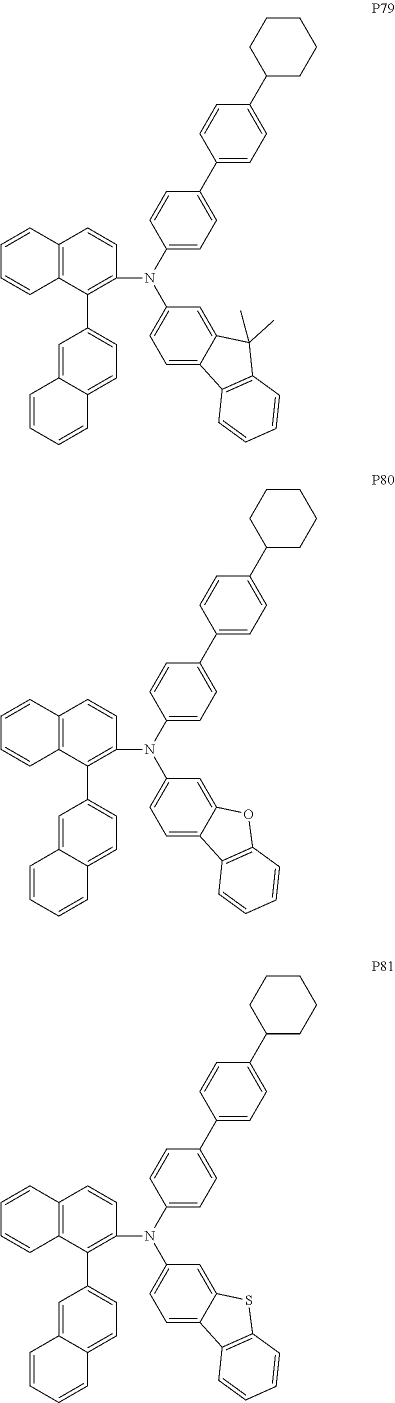 [SMILES: CC1(C)c2ccccc2-c2ccc(N(c3ccc(-c4ccc(C5CCCCC5)cc4)cc3)c3ccc4ccccc4c3-c3ccc4ccccc4c3)cc21 ; c1ccc2cc(-c3c(N(c4ccc(-c5ccc(C6CCCCC6)cc5)cc4)c4ccc5c(c4)oc4ccccc45)ccc4ccccc34)ccc2c1 ; c1ccc2cc(-c3c(N(c4ccc(-c5ccc(C6CCCCC6)cc5)cc4)c4ccc5c(c4)sc4ccccc45)ccc4ccccc34)ccc2c1]

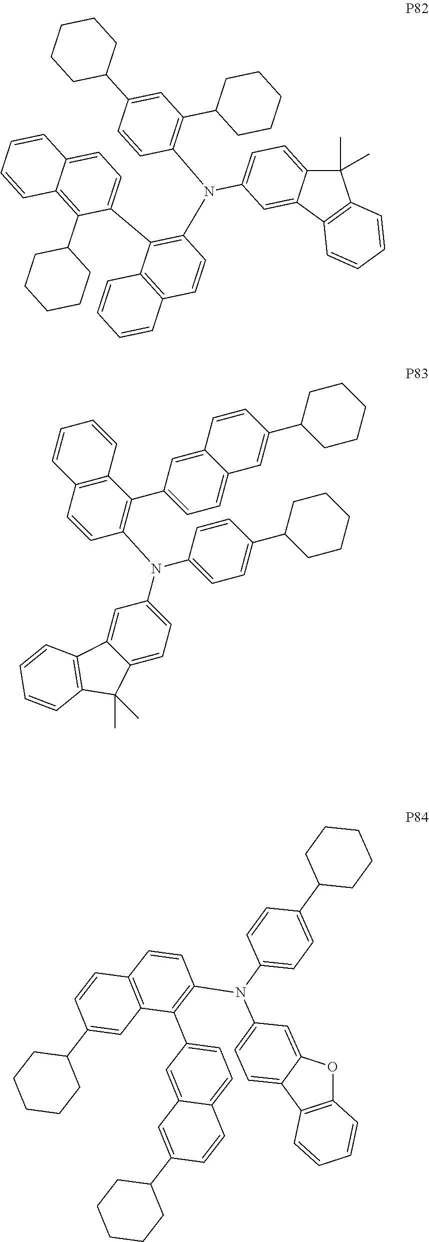 [SMILES: CC1(C)c2ccccc2-c2cc(N(c3ccc(C4CCCCC4)cc3C3CCCCC3)c3ccc4ccccc4c3-c3ccc4ccccc4c3C3CCCCC3)ccc21 ; CC1(C)c2ccccc2-c2cc(N(c3ccc(C4CCCCC4)cc3)c3ccc4ccccc4c3-c3ccc4cc(C5CCCCC5)ccc4c3)ccc21 ; c1ccc2c(c1)oc1cc(N(c3ccc(C4CCCCC4)cc3)c3ccc4ccc(C5CCCCC5)cc4c3-c3ccc4ccc(C5CCCCC5)cc4c3)ccc12]

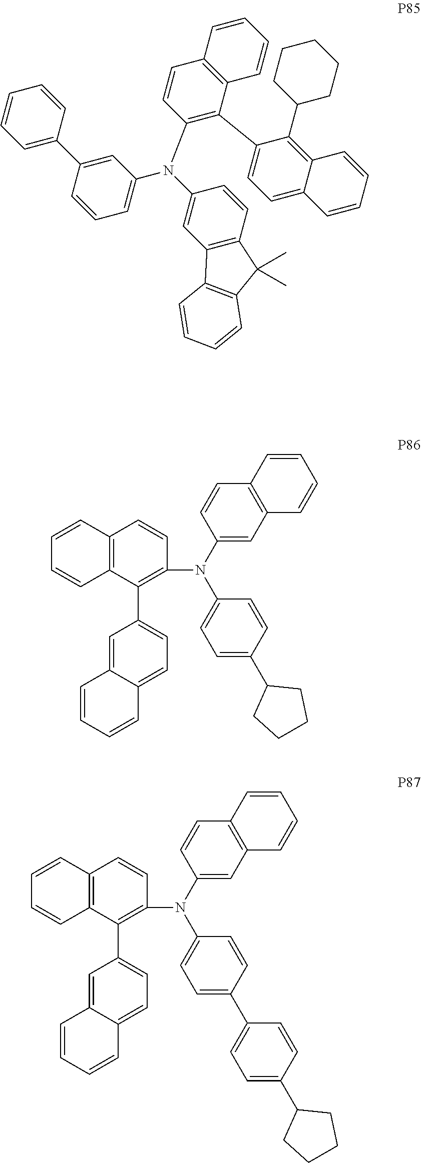 [SMILES: CC1(C)c2ccccc2-c2cc(N(c3cccc(-c4ccccc4)c3)c3ccc4ccccc4c3-c3ccc4ccccc4c3C3CCCCC3)ccc21 ; c1ccc2cc(-c3c(N(c4ccc(C5CCCC5)cc4)c4ccc5ccccc5c4)ccc4ccccc34)ccc2c1 ; c1ccc2cc(-c3c(N(c4ccc(-c5ccc(C6CCCC6)cc5)cc4)c4ccc5ccccc5c4)ccc4ccccc34)ccc2c1]

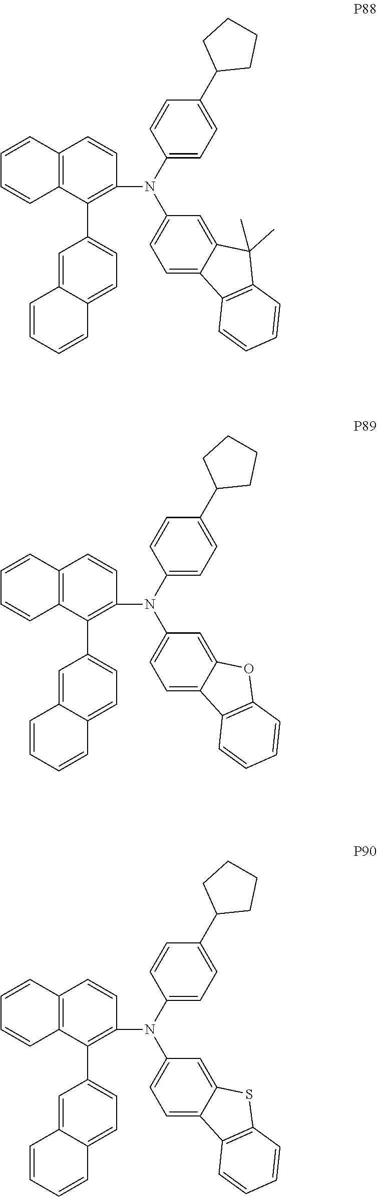 [SMILES: CC1(C)c2ccccc2-c2ccc(N(c3ccc(C4CCCC4)cc3)c3ccc4ccccc4c3-c3ccc4ccccc4c3)cc21 ; c1ccc2cc(-c3c(N(c4ccc(C5CCCC5)cc4)c4ccc5c(c4)oc4ccccc45)ccc4ccccc34)ccc2c1 ; c1ccc2cc(-c3c(N(c4ccc(C5CCCC5)cc4)c4ccc5c(c4)sc4ccccc45)ccc4ccccc34)ccc2c1]

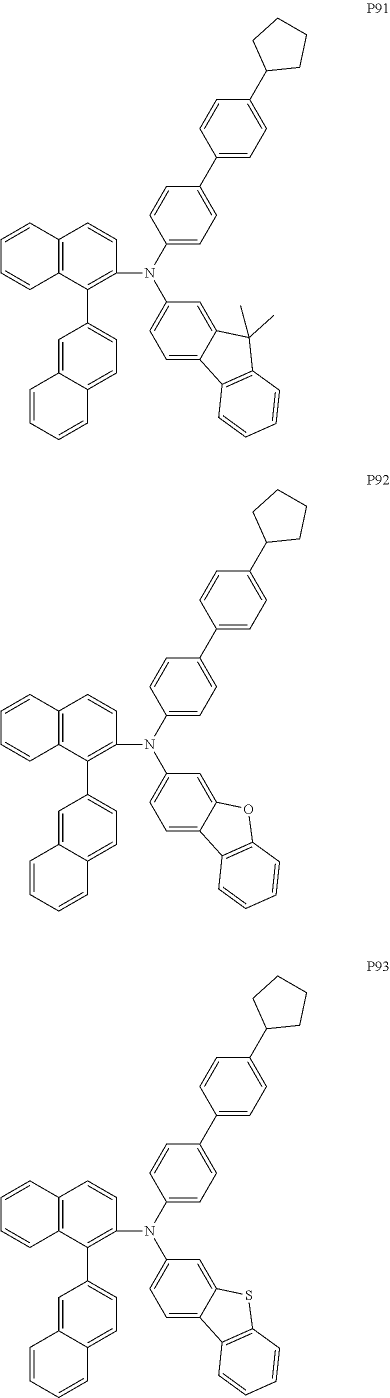 [SMILES: CC1(C)c2ccccc2-c2ccc(N(c3ccc(-c4ccc(C5CCCC5)cc4)cc3)c3ccc4ccccc4c3-c3ccc4ccccc4c3)cc21 ; c1ccc2cc(-c3c(N(c4ccc(-c5ccc(C6CCCC6)cc5)cc4)c4ccc5c(c4)oc4ccccc45)ccc4ccccc34)ccc2c1 ; c1ccc2cc(-c3c(N(c4ccc(-c5ccc(C6CCCC6)cc5)cc4)c4ccc5c(c4)sc4ccccc45)ccc4ccccc34)ccc2c1]

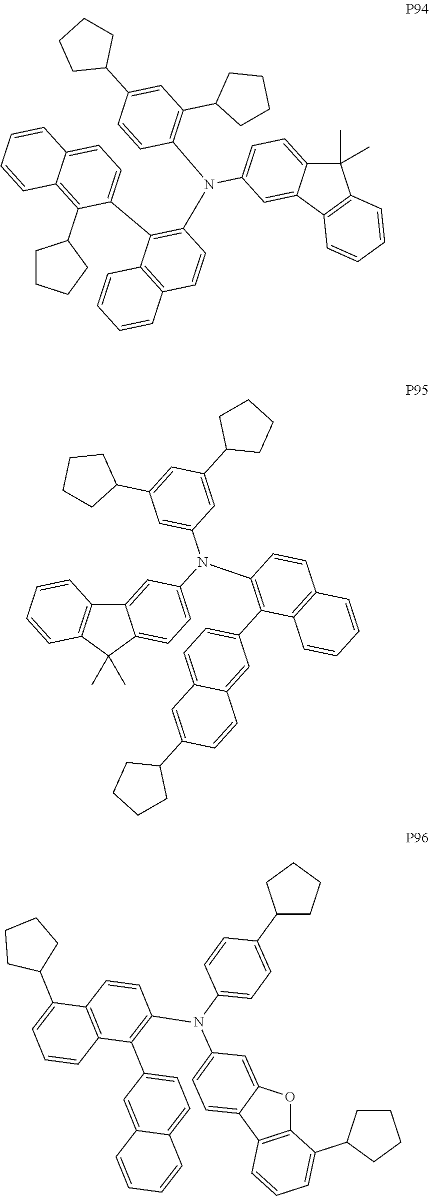 [SMILES: CC1(C)c2ccccc2-c2cc(N(c3ccc(C4CCCC4)cc3C3CCCC3)c3ccc4ccccc4c3-c3ccc4ccccc4c3C3CCCC3)ccc21 ; CC1(C)c2ccccc2-c2cc(N(c3cc(C4CCCC4)cc(C4CCCC4)c3)c3ccc4ccccc4c3-c3ccc4cc(C5CCCC5)ccc4c3)ccc21 ; c1ccc2cc(-c3c(N(c4ccc(C5CCCC5)cc4)c4ccc5c(c4)oc4c(C6CCCC6)cccc45)ccc4c(C5CCCC5)cccc34)ccc2c1]

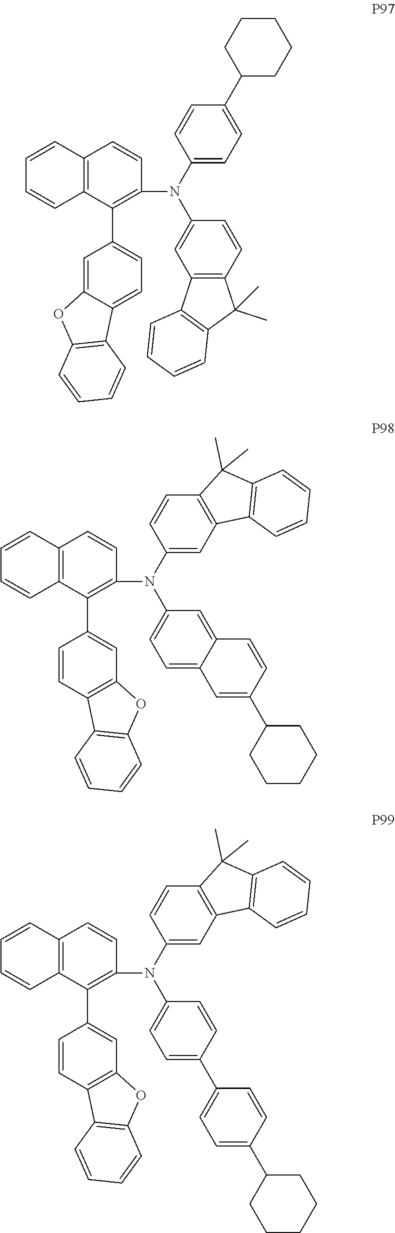 [SMILES: CC1(C)c2ccccc2-c2cc(N(c3ccc(C4CCCCC4)cc3)c3ccc4ccccc4c3-c3ccc4c(c3)oc3ccccc34)ccc21 ; CC1(C)c2ccccc2-c2cc(N(c3ccc4cc(C5CCCCC5)ccc4c3)c3ccc4ccccc4c3-c3ccc4c(c3)oc3ccccc34)ccc21 ; CC1(C)c2ccccc2-c2cc(N(c3ccc(-c4ccc(C5CCCCC5)cc4)cc3)c3ccc4ccccc4c3-c3ccc4c(c3)oc3ccccc34)ccc21]

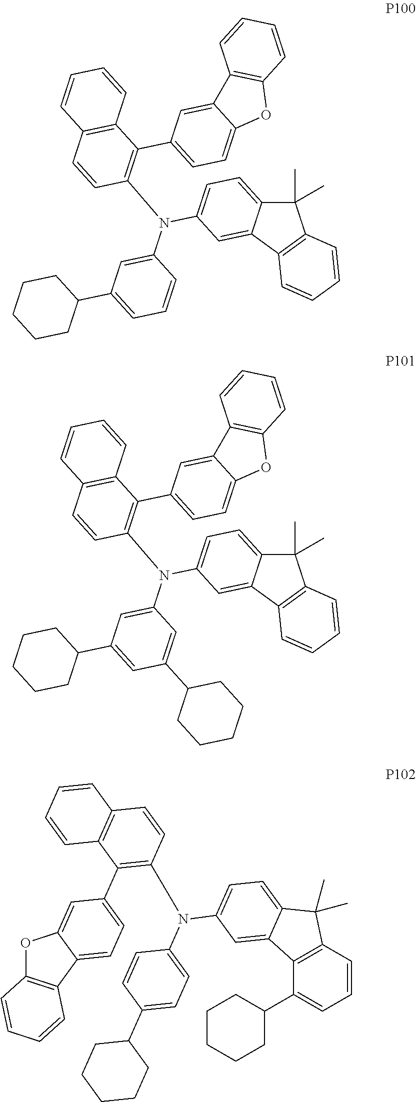 [SMILES: CC1(C)c2ccccc2-c2cc(N(c3cccc(C4CCCCC4)c3)c3ccc4ccccc4c3-c3ccc4oc5ccccc5c4c3)ccc21 ; CC1(C)c2ccccc2-c2cc(N(c3cc(C4CCCCC4)cc(C4CCCCC4)c3)c3ccc4ccccc4c3-c3ccc4oc5ccccc5c4c3)ccc21 ; CC1(C)c2ccc(N(c3ccc(C4CCCCC4)cc3)c3ccc4ccccc4c3-c3ccc4c(c3)oc3ccccc34)cc2-c2c(C3CCCCC3)cccc21]

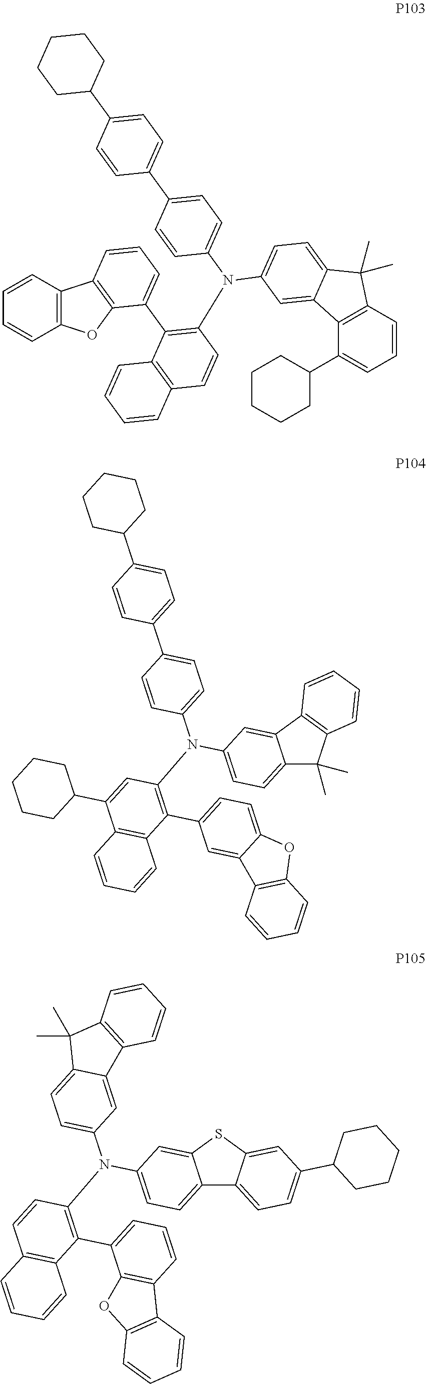 [SMILES: CC1(C)c2ccc(N(c3ccc(-c4ccc(C5CCCCC5)cc4)cc3)c3ccc4ccccc4c3-c3cccc4c3oc3ccccc34)cc2-c2c(C3CCCCC3)cccc21 ; CC1(C)c2ccccc2-c2cc(N(c3ccc(-c4ccc(C5CCCCC5)cc4)cc3)c3cc(C4CCCCC4)c4ccccc4c3-c3ccc4oc5ccccc5c4c3)ccc21 ; CC1(C)c2ccccc2-c2cc(N(c3ccc4c(c3)sc3cc(C5CCCCC5)ccc34)c3ccc4ccccc4c3-c3cccc4c3oc3ccccc34)ccc21]

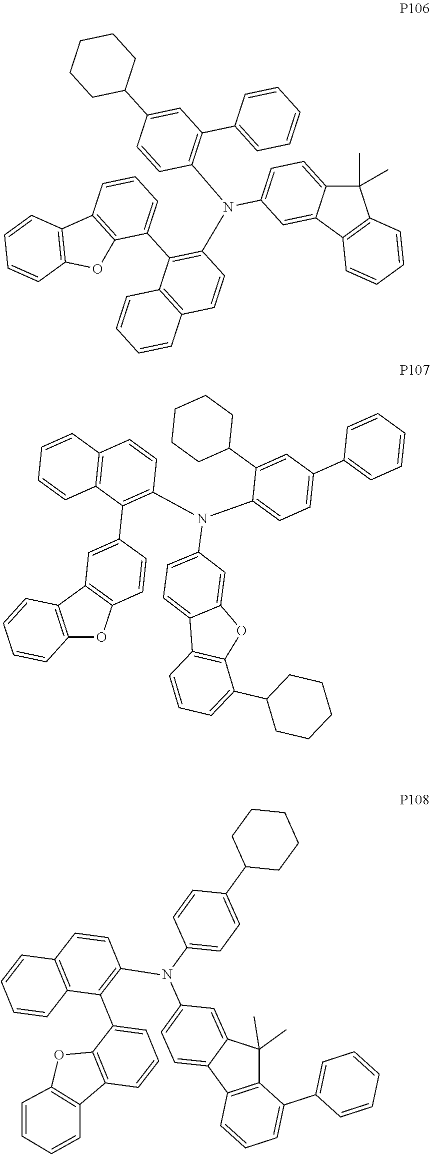 [SMILES: CC1(C)c2ccccc2-c2cc(N(c3ccc(C4CCCCC4)cc3-c3ccccc3)c3ccc4ccccc4c3-c3cccc4c3oc3ccccc34)ccc21 ; c1ccc(-c2ccc(N(c3ccc4c(c3)oc3c(C5CCCCC5)cccc34)c3ccc4ccccc4c3-c3ccc4oc5ccccc5c4c3)c(C3CCCCC3)c2)cc1 ; CC1(C)c2cc(N(c3ccc(C4CCCCC4)cc3)c3ccc4ccccc4c3-c3cccc4c3oc3ccccc34)ccc2-c2cccc(-c3ccccc3)c21]

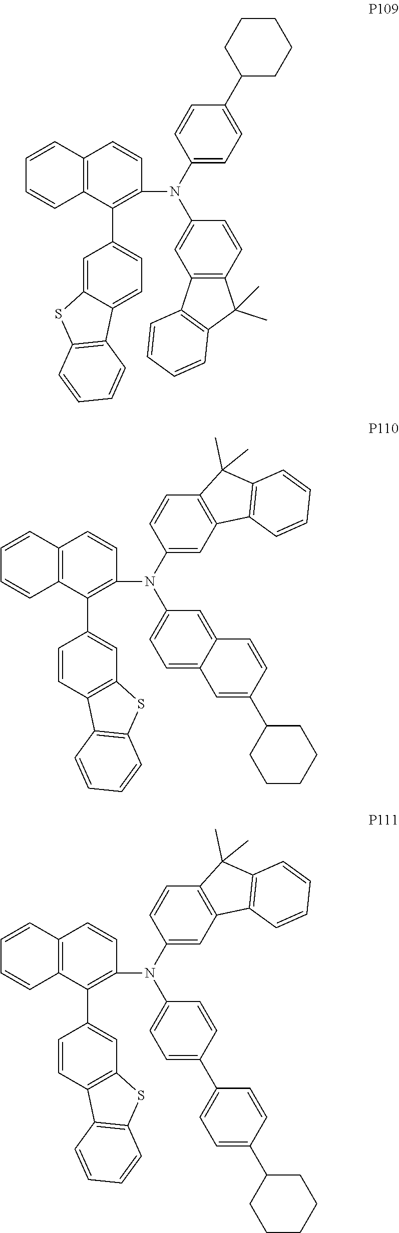 [SMILES: CC1(C)c2ccccc2-c2cc(N(c3ccc(C4CCCCC4)cc3)c3ccc4ccccc4c3-c3ccc4c(c3)sc3ccccc34)ccc21 ; CC1(C)c2ccccc2-c2cc(N(c3ccc4cc(C5CCCCC5)ccc4c3)c3ccc4ccccc4c3-c3ccc4c(c3)sc3ccccc34)ccc21 ; CC1(C)c2ccccc2-c2cc(N(c3ccc(-c4ccc(C5CCCCC5)cc4)cc3)c3ccc4ccccc4c3-c3ccc4c(c3)sc3ccccc34)ccc21]

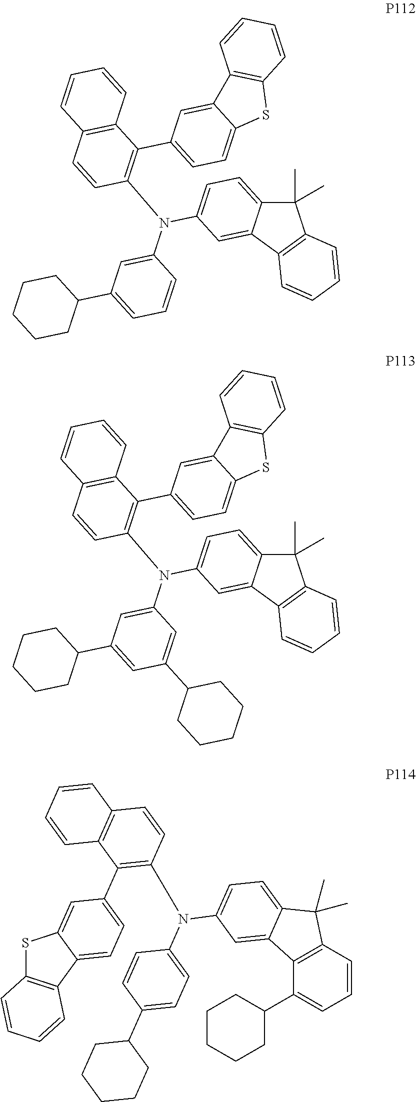 [SMILES: CC1(C)c2ccccc2-c2cc(N(c3cccc(C4CCCCC4)c3)c3ccc4ccccc4c3-c3ccc4sc5ccccc5c4c3)ccc21 ; CC1(C)c2ccccc2-c2cc(N(c3cc(C4CCCCC4)cc(C4CCCCC4)c3)c3ccc4ccccc4c3-c3ccc4sc5ccccc5c4c3)ccc21 ; CC1(C)c2ccc(N(c3ccc(C4CCCCC4)cc3)c3ccc4ccccc4c3-c3ccc4c(c3)sc3ccccc34)cc2-c2c(C3CCCCC3)cccc21]

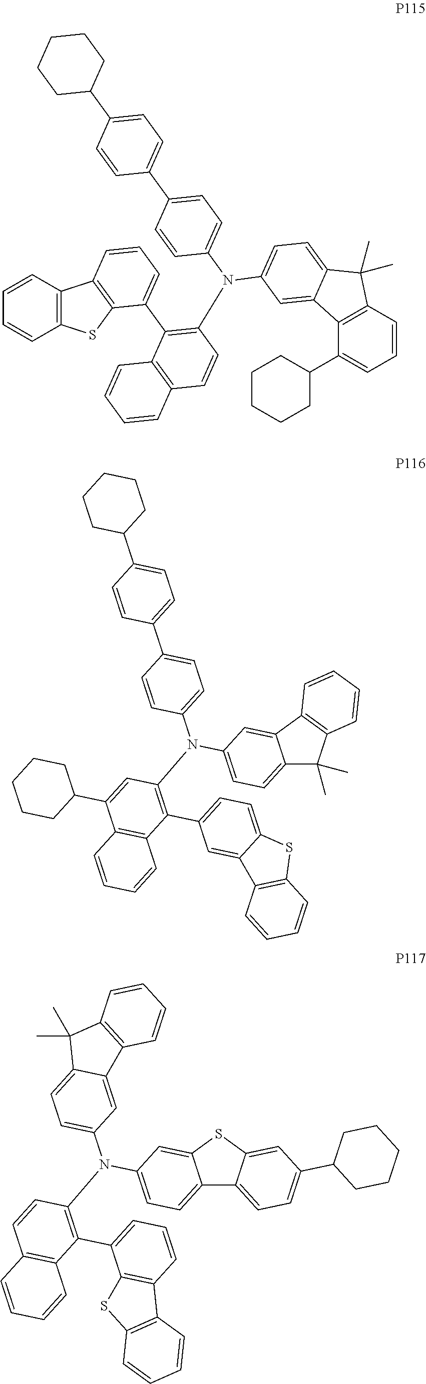 [SMILES: CC1(C)c2ccc(N(c3ccc(-c4ccc(C5CCCCC5)cc4)cc3)c3ccc4ccccc4c3-c3cccc4c3sc3ccccc34)cc2-c2c(C3CCCCC3)cccc21 ; CC1(C)c2ccccc2-c2cc(N(c3ccc(-c4ccc(C5CCCCC5)cc4)cc3)c3cc(C4CCCCC4)c4ccccc4c3-c3ccc4sc5ccccc5c4c3)ccc21 ; CC1(C)c2ccccc2-c2cc(N(c3ccc4c(c3)sc3cc(C5CCCCC5)ccc34)c3ccc4ccccc4c3-c3cccc4c3sc3ccccc34)ccc21]

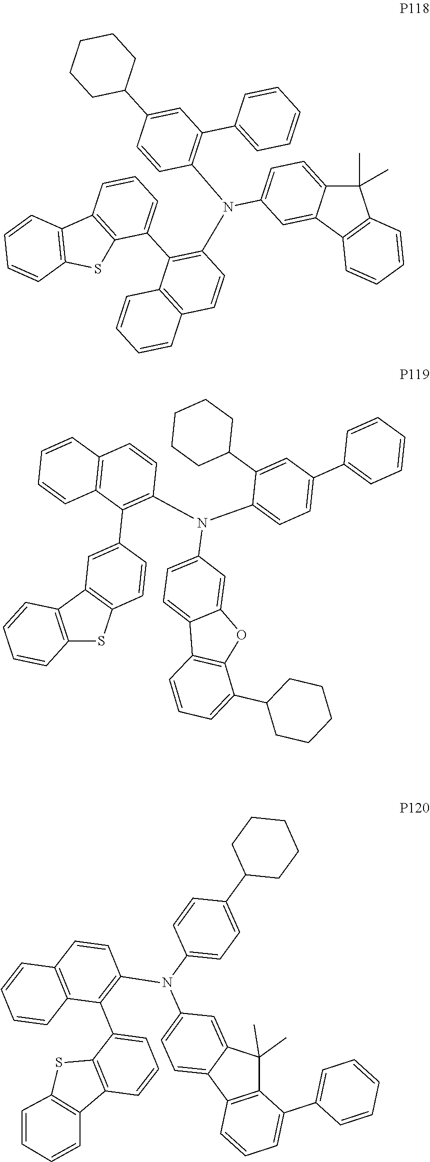 [SMILES: CC1(C)c2ccccc2-c2cc(N(c3ccc(C4CCCCC4)cc3-c3ccccc3)c3ccc4ccccc4c3-c3cccc4c3sc3ccccc34)ccc21 ; c1ccc(-c2ccc(N(c3ccc4c(c3)oc3c(C5CCCCC5)cccc34)c3ccc4ccccc4c3-c3ccc4sc5ccccc5c4c3)c(C3CCCCC3)c2)cc1 ; CC1(C)c2cc(N(c3ccc(C4CCCCC4)cc3)c3ccc4ccccc4c3-c3cccc4c3sc3ccccc34)ccc2-c2cccc(-c3ccccc3)c21]

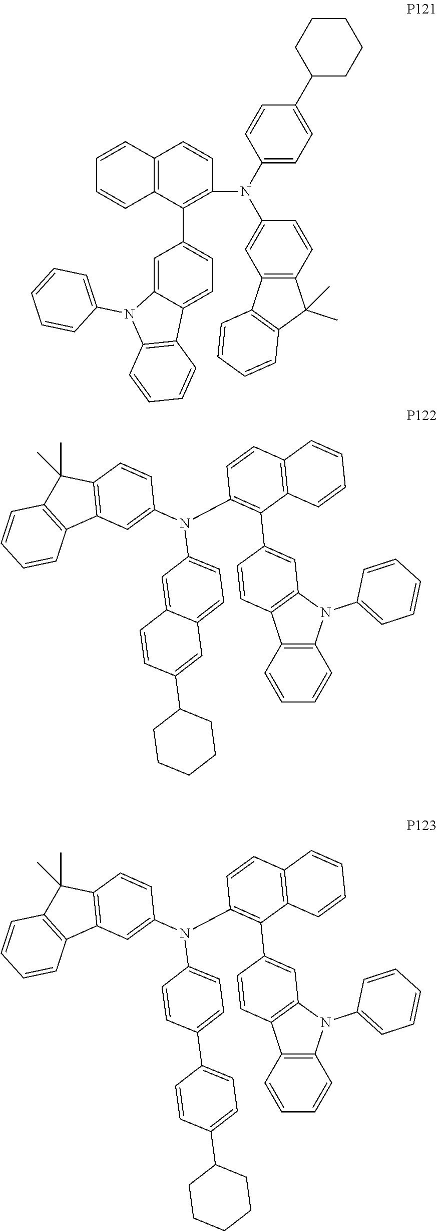 [SMILES: CC1(C)c2ccccc2-c2cc(N(c3ccc(C4CCCCC4)cc3)c3ccc4ccccc4c3-c3ccc4c5ccccc5n(-c5ccccc5)c4c3)ccc21 ; CC1(C)c2ccccc2-c2cc(N(c3ccc4cc(C5CCCCC5)ccc4c3)c3ccc4ccccc4c3-c3ccc4c5ccccc5n(-c5ccccc5)c4c3)ccc21 ; CC1(C)c2ccccc2-c2cc(N(c3ccc(-c4ccc(C5CCCCC5)cc4)cc3)c3ccc4ccccc4c3-c3ccc4c5ccccc5n(-c5ccccc5)c4c3)ccc21]

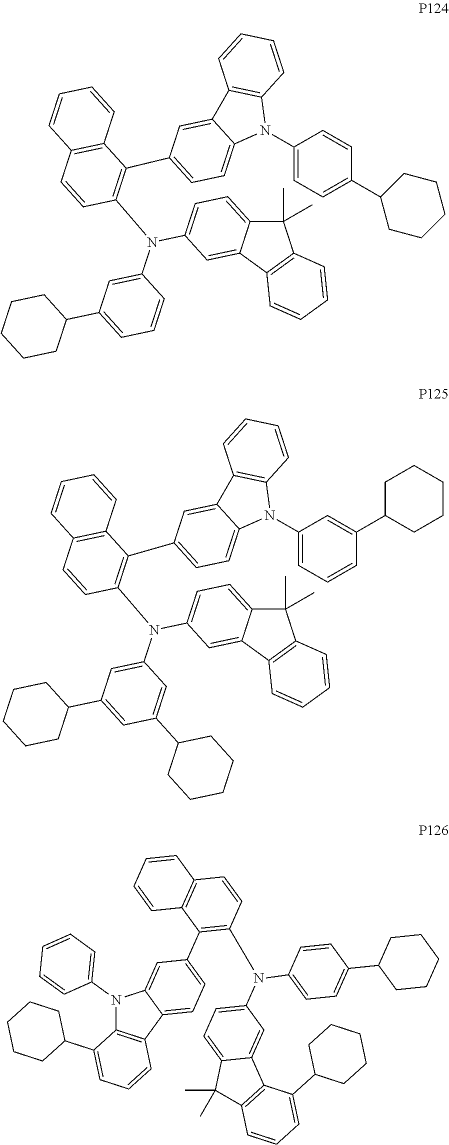 [SMILES: CC1(C)c2ccccc2-c2cc(N(c3cccc(C4CCCCC4)c3)c3ccc4ccccc4c3-c3ccc4c(c3)c3ccccc3n4-c3ccc(C4CCCCC4)cc3)ccc21 ; CC1(C)c2ccccc2-c2cc(N(c3cc(C4CCCCC4)cc(C4CCCCC4)c3)c3ccc4ccccc4c3-c3ccc4c(c3)c3ccccc3n4-c3cccc(C4CCCCC4)c3)ccc21 ; CC1(C)c2ccc(N(c3ccc(C4CCCCC4)cc3)c3ccc4ccccc4c3-c3ccc4c5cccc(C6CCCCC6)c5n(-c5ccccc5)c4c3)cc2-c2c(C3CCCCC3)cccc21]

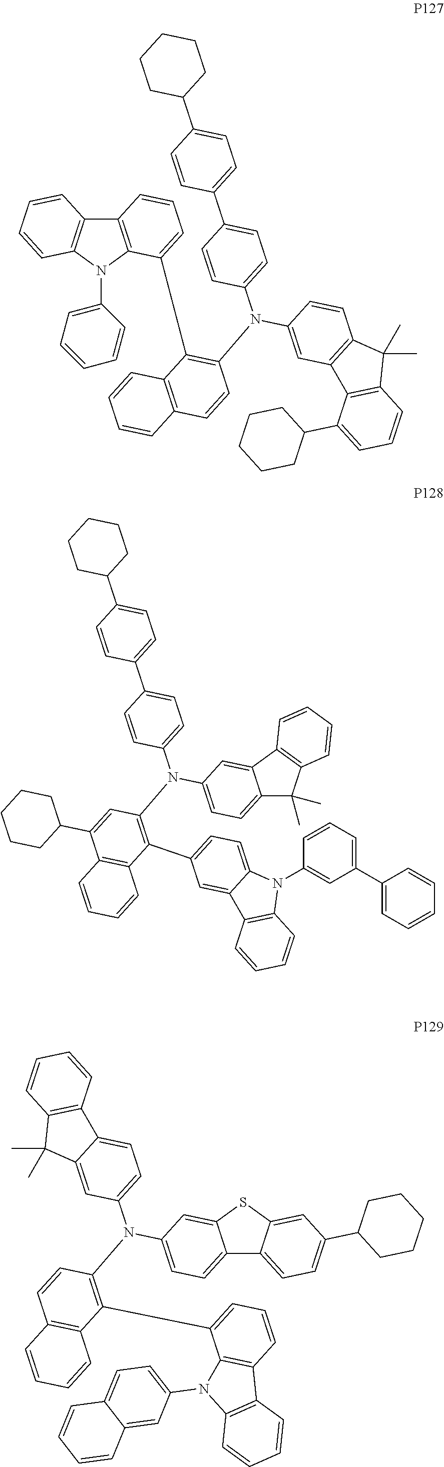

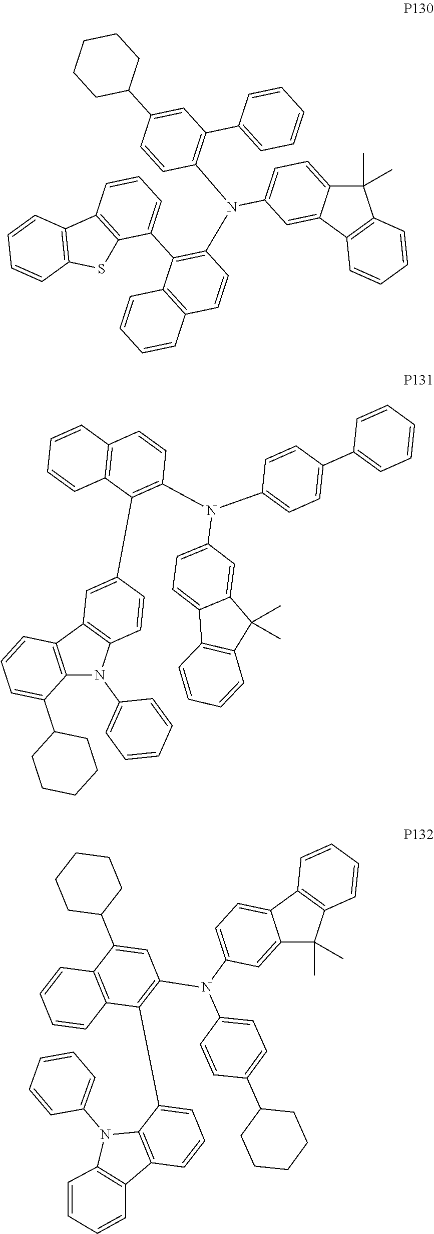 [SMILES: CC1(C)c2ccccc2-c2cc(N(c3ccc(C4CCCCC4)cc3-c3ccccc3)c3ccc4ccccc4c3-c3cccc4c3sc3ccccc34)ccc21 ; CC1(C)c2ccccc2-c2ccc(N(c3ccc(-c4ccccc4)cc3)c3ccc4ccccc4c3-c3ccc4c(c3)c3cccc(C5CCCCC5)c3n4-c3ccccc3)cc21 ; CC1(C)c2ccccc2-c2ccc(N(c3ccc(C4CCCCC4)cc3)c3cc(C4CCCCC4)c4ccccc4c3-c3cccc4c5ccccc5n(-c5ccccc5)c34)cc21]

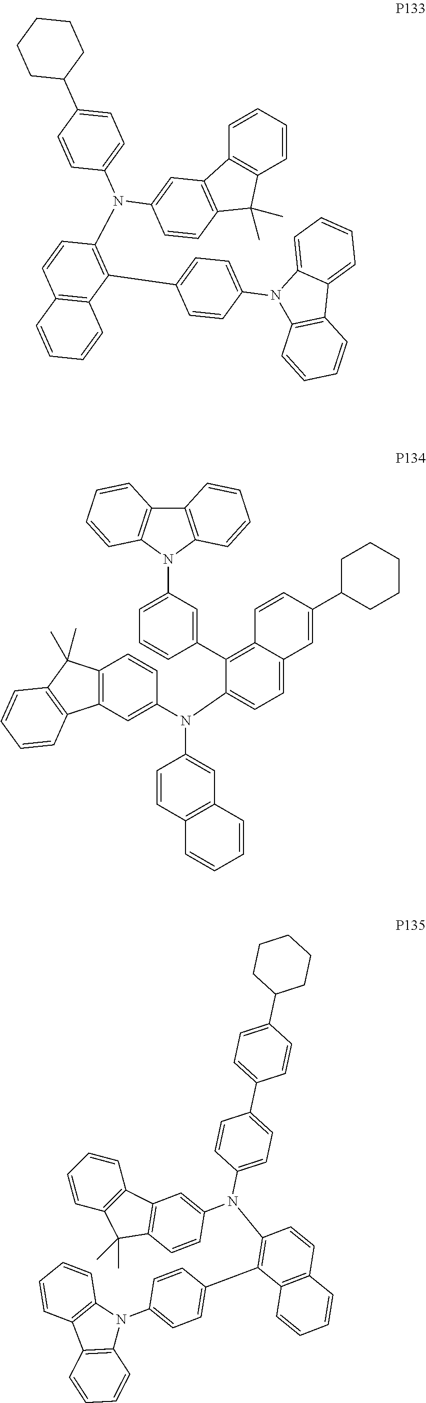 [SMILES: CC1(C)c2ccccc2-c2cc(N(c3ccc(C4CCCCC4)cc3)c3ccc4ccccc4c3-c3ccc(-n4c5ccccc5c5ccccc54)cc3)ccc21 ; CC1(C)c2ccccc2-c2cc(N(c3ccc4ccccc4c3)c3ccc4cc(C5CCCCC5)ccc4c3-c3cccc(-n4c5ccccc5c5ccccc54)c3)ccc21 ; CC1(C)c2ccccc2-c2cc(N(c3ccc(-c4ccc(C5CCCCC5)cc4)cc3)c3ccc4ccccc4c3-c3ccc(-n4c5ccccc5c5ccccc54)cc3)ccc21]

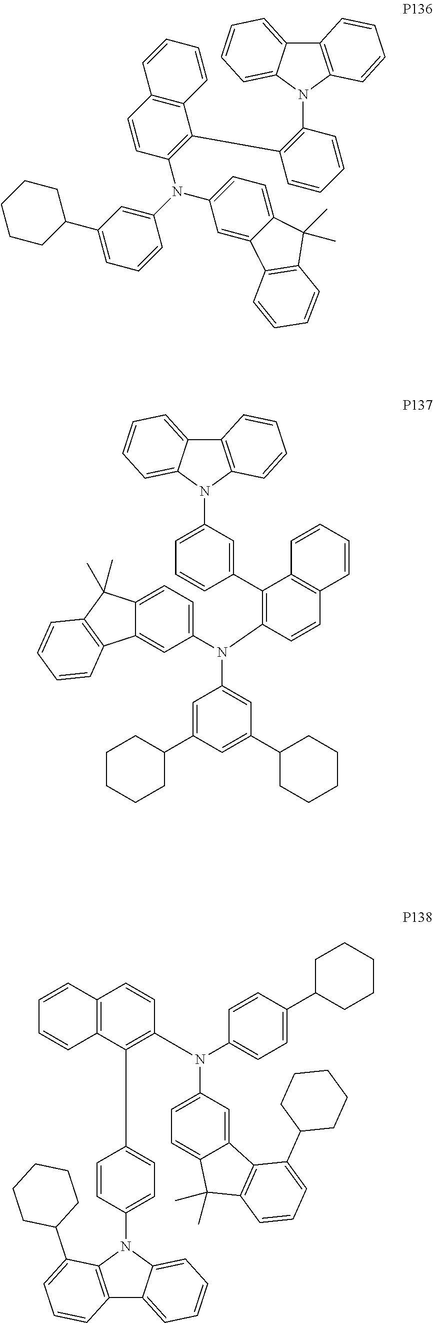 [SMILES: CC1(C)c2ccccc2-c2cc(N(c3cccc(C4CCCCC4)c3)c3ccc4ccccc4c3-c3ccccc3-n3c4ccccc4c4ccccc43)ccc21 ; CC1(C)c2ccccc2-c2cc(N(c3cc(C4CCCCC4)cc(C4CCCCC4)c3)c3ccc4ccccc4c3-c3cccc(-n4c5ccccc5c5ccccc54)c3)ccc21 ; CC1(C)c2ccc(N(c3ccc(C4CCCCC4)cc3)c3ccc4ccccc4c3-c3ccc(-n4c5ccccc5c5cccc(C6CCCCC6)c54)cc3)cc2-c2c(C3CCCCC3)cccc21]

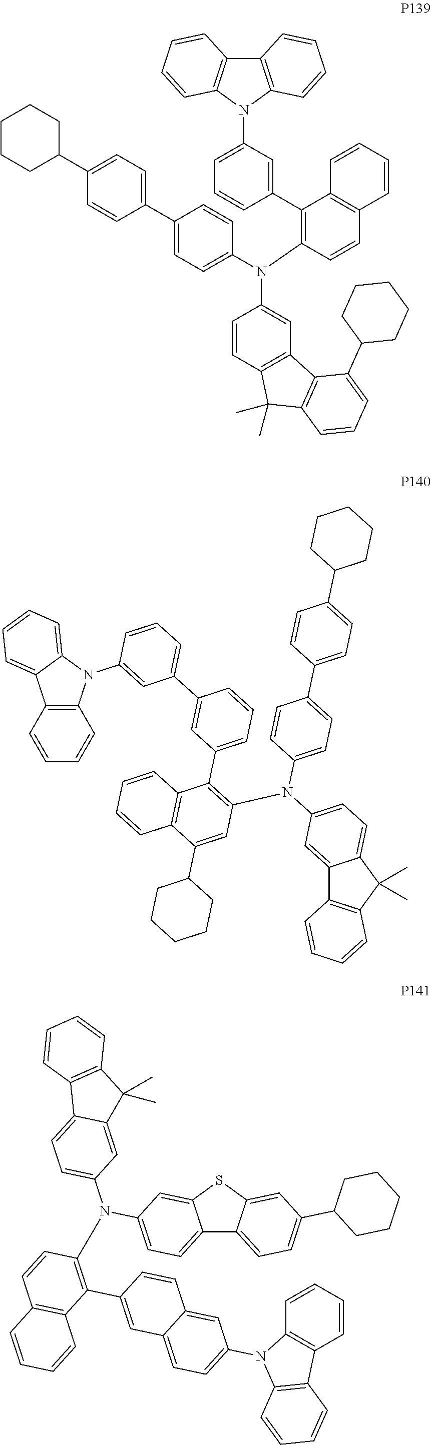 [SMILES: CC1(C)c2ccc(N(c3ccc(-c4ccc(C5CCCCC5)cc4)cc3)c3ccc4ccccc4c3-c3cccc(-n4c5ccccc5c5ccccc54)c3)cc2-c2c(C3CCCCC3)cccc21 ; CC1(C)c2ccccc2-c2cc(N(c3ccc(-c4ccc(C5CCCCC5)cc4)cc3)c3cc(C4CCCCC4)c4ccccc4c3-c3cccc(-c4cccc(-n5c6ccccc6c6ccccc65)c4)c3)ccc21 ; CC1(C)c2ccccc2-c2ccc(N(c3ccc4c(c3)sc3cc(C5CCCCC5)ccc34)c3ccc4ccccc4c3-c3ccc4cc(-n5c6ccccc6c6ccccc65)ccc4c3)cc21]

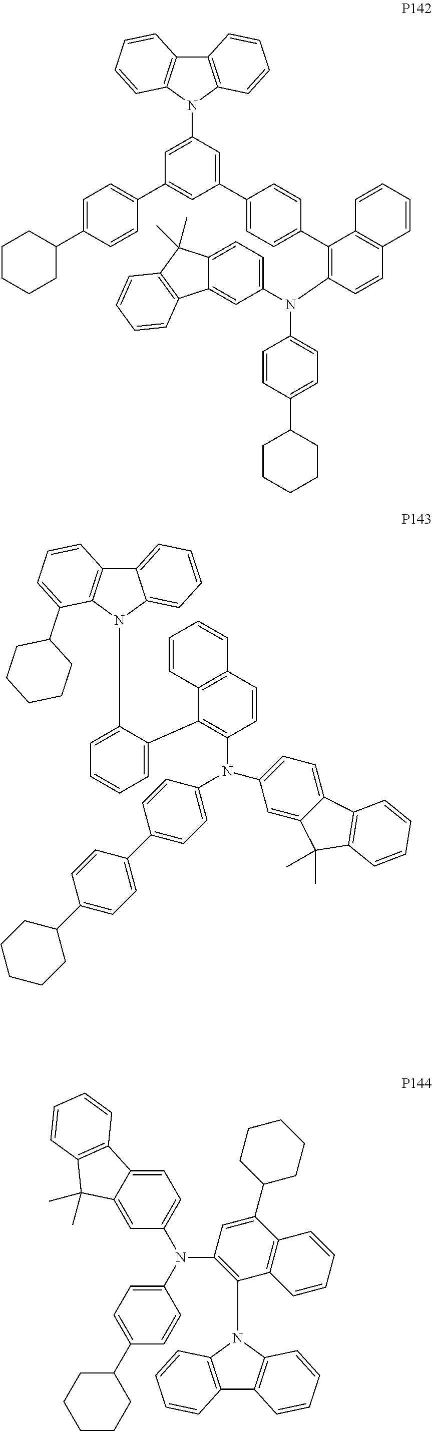 [SMILES: CC1(C)c2ccccc2-c2cc(N(c3ccc(C4CCCCC4)cc3)c3ccc4ccccc4c3-c3ccc(-c4cc(-c5ccc(C6CCCCC6)cc5)cc(-n5c6ccccc6c6ccccc65)c4)cc3)ccc21 ; CC1(C)c2ccccc2-c2ccc(N(c3ccc(-c4ccc(C5CCCCC5)cc4)cc3)c3ccc4ccccc4c3-c3ccccc3-n3c4ccccc4c4cccc(C5CCCCC5)c43)cc21 ; CC1(C)c2ccccc2-c2ccc(N(c3ccc(C4CCCCC4)cc3)c3cc(C4CCCCC4)c4ccccc4c3-n3c4ccccc4c4ccccc43)cc21]

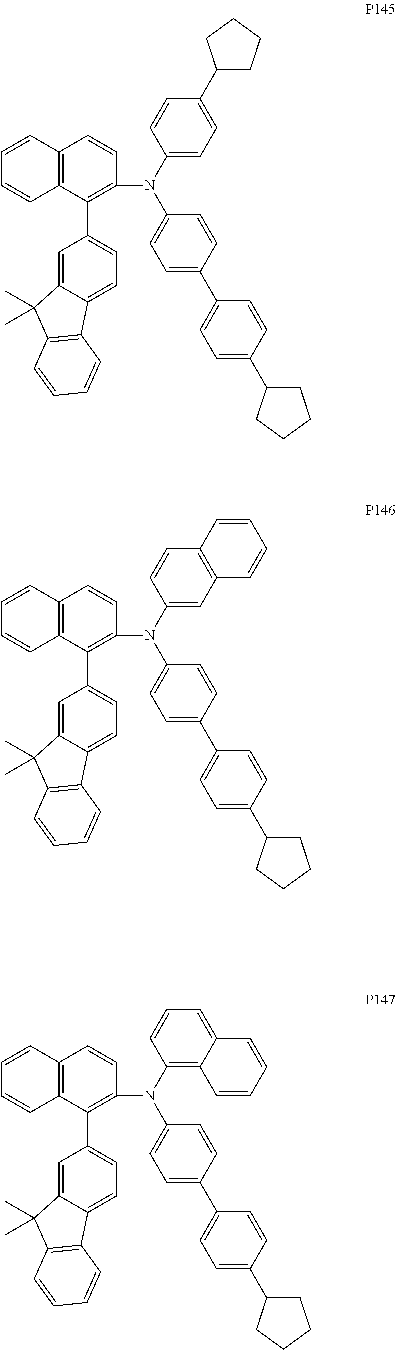 [SMILES: CC1(C)c2ccccc2-c2ccc(-c3c(N(c4ccc(-c5ccc(C6CCCC6)cc5)cc4)c4ccc(C5CCCC5)cc4)ccc4ccccc34)cc21 ; CC1(C)c2ccccc2-c2ccc(-c3c(N(c4ccc(-c5ccc(C6CCCC6)cc5)cc4)c4ccc5ccccc5c4)ccc4ccccc34)cc21 ; CC1(C)c2ccccc2-c2ccc(-c3c(N(c4ccc(-c5ccc(C6CCCC6)cc5)cc4)c4cccc5ccccc45)ccc4ccccc34)cc21]

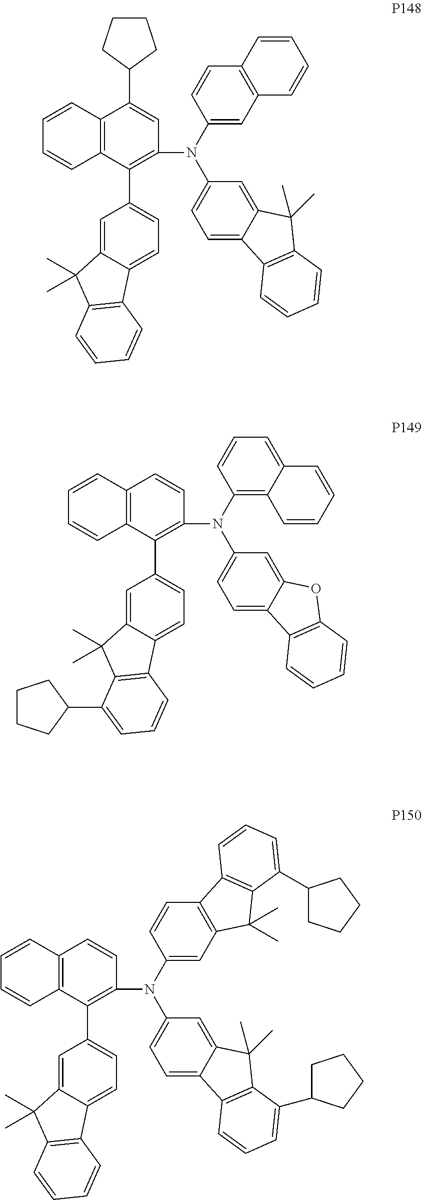 [SMILES: CC1(C)c2ccccc2-c2ccc(-c3c(N(c4ccc5c(c4)C(C)(C)c4ccccc4-5)c4ccc5ccccc5c4)cc(C4CCCC4)c4ccccc34)cc21 ; CC1(C)c2cc(-c3c(N(c4ccc5c(c4)oc4ccccc45)c4cccc5ccccc45)ccc4ccccc34)ccc2-c2cccc(C3CCCC3)c21 ; CC1(C)c2ccccc2-c2ccc(-c3c(N(c4ccc5c(c4)C(C)(C)c4c-5cccc4C4CCCC4)c4ccc5c(c4)C(C)(C)c4c-5cccc4C4CCCC4)ccc4ccccc34)cc21]

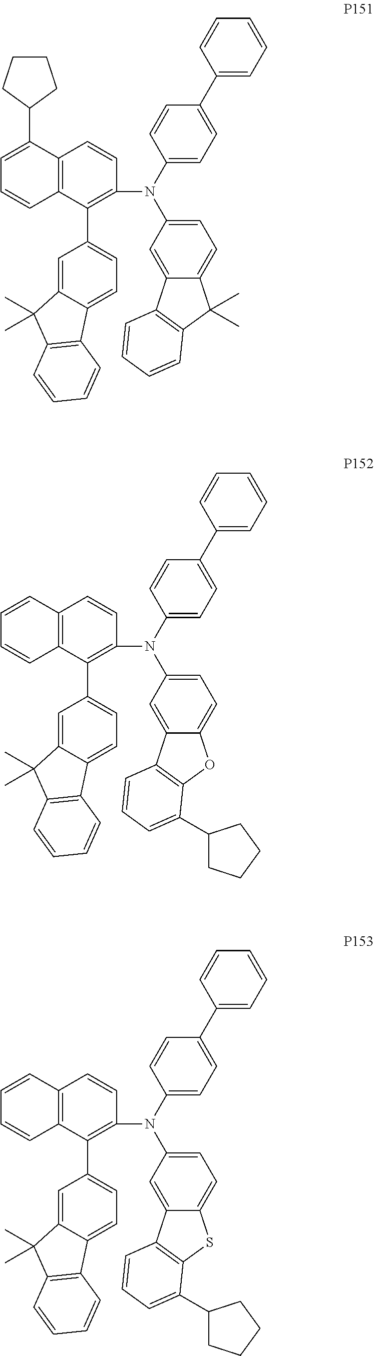 [SMILES: CC1(C)c2ccccc2-c2cc(N(c3ccc(-c4ccccc4)cc3)c3ccc4c(C5CCCC5)cccc4c3-c3ccc4c(c3)C(C)(C)c3ccccc3-4)ccc21 ; CC1(C)c2ccccc2-c2ccc(-c3c(N(c4ccc(-c5ccccc5)cc4)c4ccc5oc6c(C7CCCC7)cccc6c5c4)ccc4ccccc34)cc21 ; CC1(C)c2ccccc2-c2ccc(-c3c(N(c4ccc(-c5ccccc5)cc4)c4ccc5sc6c(C7CCCC7)cccc6c5c4)ccc4ccccc34)cc21]

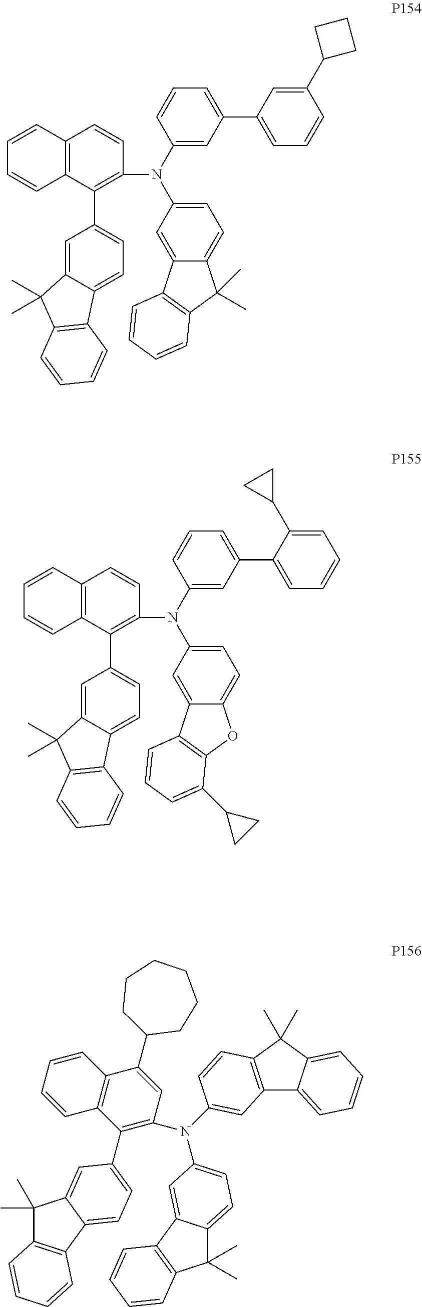 [SMILES: CC1(C)c2ccccc2-c2cc(N(c3cccc(-c4cccc(C5CCC5)c4)c3)c3ccc4ccccc4c3-c3ccc4c(c3)C(C)(C)c3ccccc3-4)ccc21 ; CC1(C)c2ccccc2-c2ccc(-c3c(N(c4cccc(-c5ccccc5C5CC5)c4)c4ccc5oc6c(C7CC7)cccc6c5c4)ccc4ccccc34)cc21 ; CC1(C)c2ccccc2-c2cc(N(c3ccc4c(c3)-c3ccccc3C4(C)C)c3cc(C4CCCCCC4)c4ccccc4c3-c3ccc4c(c3)C(C)(C)c3ccccc3-4)ccc21]

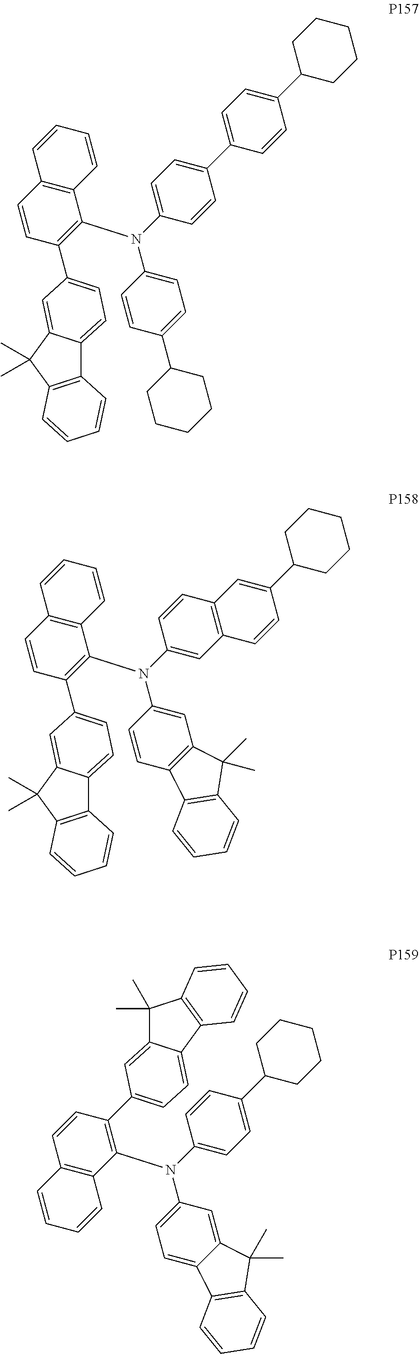 [SMILES: CC1(C)c2ccccc2-c2ccc(-c3ccc4ccccc4c3N(c3ccc(-c4ccc(C5CCCCC5)cc4)cc3)c3ccc(C4CCCCC4)cc3)cc21 ; CC1(C)c2ccccc2-c2ccc(-c3ccc4ccccc4c3N(c3ccc4c(c3)C(C)(C)c3ccccc3-4)c3ccc4cc(C5CCCCC5)ccc4c3)cc21 ; CC1(C)c2ccccc2-c2ccc(-c3ccc4ccccc4c3N(c3ccc(C4CCCCC4)cc3)c3ccc4c(c3)C(C)(C)c3ccccc3-4)cc21]

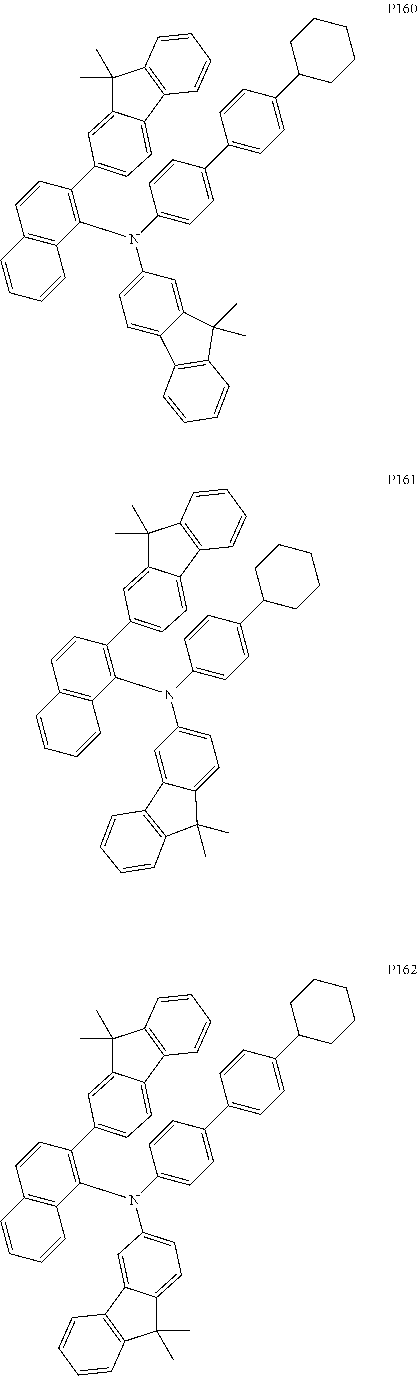 [SMILES: CC1(C)c2ccccc2-c2ccc(-c3ccc4ccccc4c3N(c3ccc(-c4ccc(C5CCCCC5)cc4)cc3)c3ccc4c(c3)C(C)(C)c3ccccc3-4)cc21 ; CC1(C)c2ccccc2-c2cc(N(c3ccc(C4CCCCC4)cc3)c3c(-c4ccc5c(c4)C(C)(C)c4ccccc4-5)ccc4ccccc34)ccc21 ; CC1(C)c2ccccc2-c2cc(N(c3ccc(-c4ccc(C5CCCCC5)cc4)cc3)c3c(-c4ccc5c(c4)C(C)(C)c4ccccc4-5)ccc4ccccc34)ccc21]

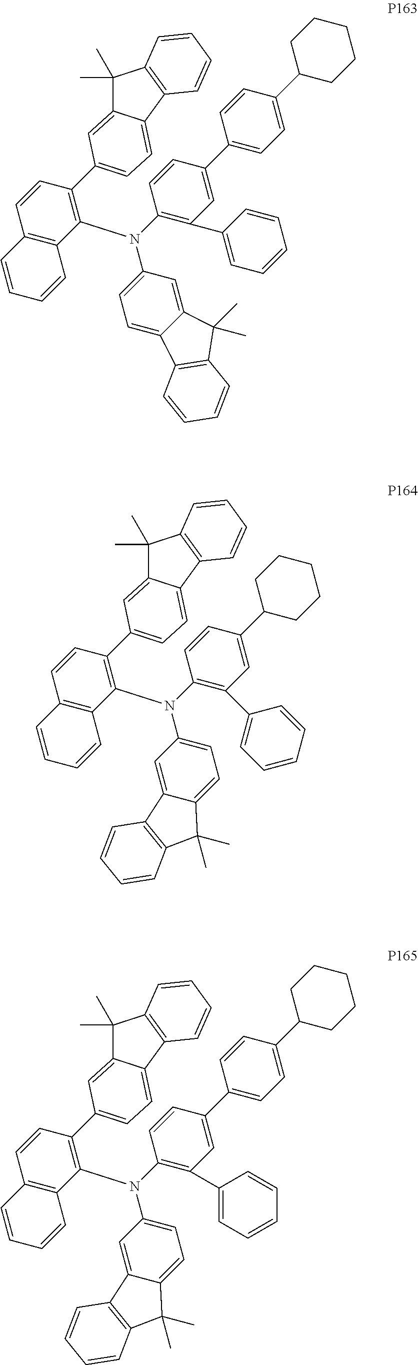 [SMILES: CC1(C)c2ccccc2-c2ccc(-c3ccc4ccccc4c3N(c3ccc4c(c3)C(C)(C)c3ccccc3-4)c3ccc(-c4ccc(C5CCCCC5)cc4)cc3-c3ccccc3)cc21 ; CC1(C)c2ccccc2-c2cc(N(c3ccc(C4CCCCC4)cc3-c3ccccc3)c3c(-c4ccc5c(c4)C(C)(C)c4ccccc4-5)ccc4ccccc34)ccc21 ; CC1(C)c2ccccc2-c2cc(N(c3ccc(-c4ccc(C5CCCCC5)cc4)cc3-c3ccccc3)c3c(-c4ccc5c(c4)C(C)(C)c4ccccc4-5)ccc4ccccc34)ccc21]

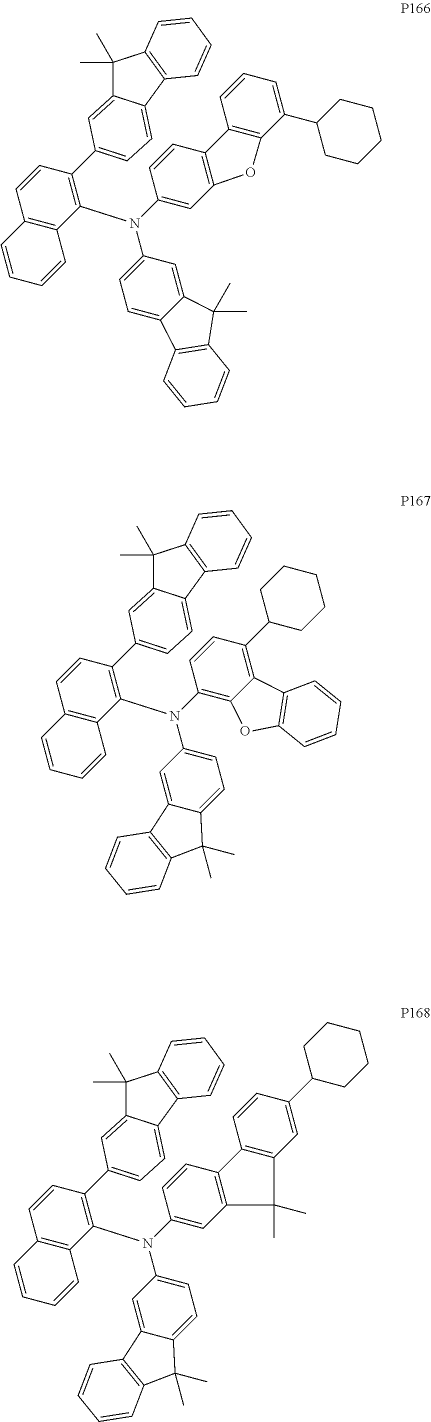 [SMILES: CC1(C)c2ccccc2-c2ccc(-c3ccc4ccccc4c3N(c3ccc4c(c3)C(C)(C)c3ccccc3-4)c3ccc4c(c3)oc3c(C5CCCCC5)cccc34)cc21 ; CC1(C)c2ccccc2-c2cc(N(c3c(-c4ccc5c(c4)C(C)(C)c4ccccc4-5)ccc4ccccc34)c3ccc(C4CCCCC4)c4c3oc3ccccc34)ccc21 ; CC1(C)c2ccccc2-c2cc(N(c3ccc4c(c3)C(C)(C)c3cc(C5CCCCC5)ccc3-4)c3c(-c4ccc5c(c4)C(C)(C)c4ccccc4-5)ccc4ccccc34)ccc21]

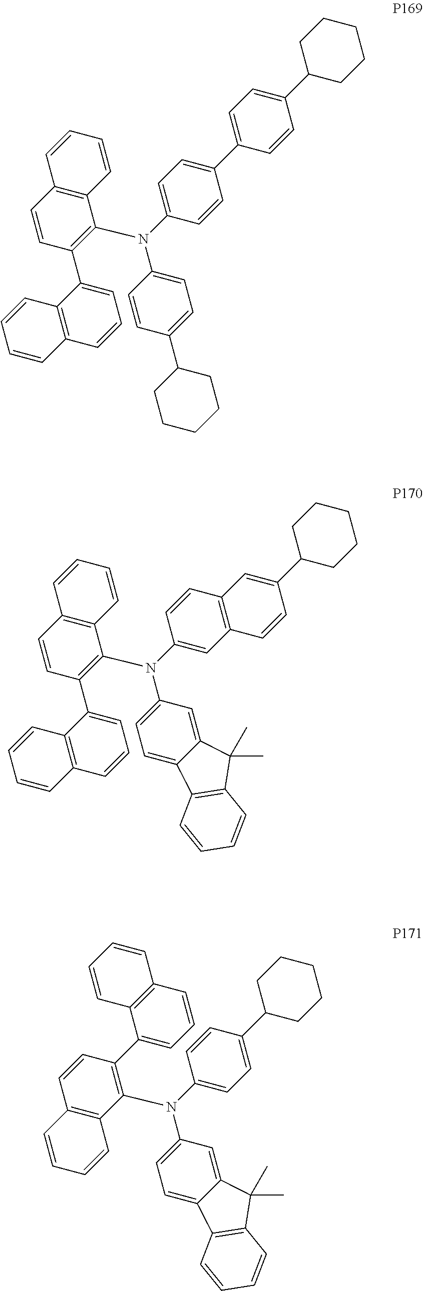 [SMILES: c1ccc2c(-c3ccc4ccccc4c3N(c3ccc(-c4ccc(C5CCCCC5)cc4)cc3)c3ccc(C4CCCCC4)cc3)cccc2c1 ; CC1(C)c2ccccc2-c2ccc(N(c3ccc4cc(C5CCCCC5)ccc4c3)c3c(-c4cccc5ccccc45)ccc4ccccc34)cc21 ; CC1(C)c2ccccc2-c2ccc(N(c3ccc(C4CCCCC4)cc3)c3c(-c4cccc5ccccc45)ccc4ccccc34)cc21]

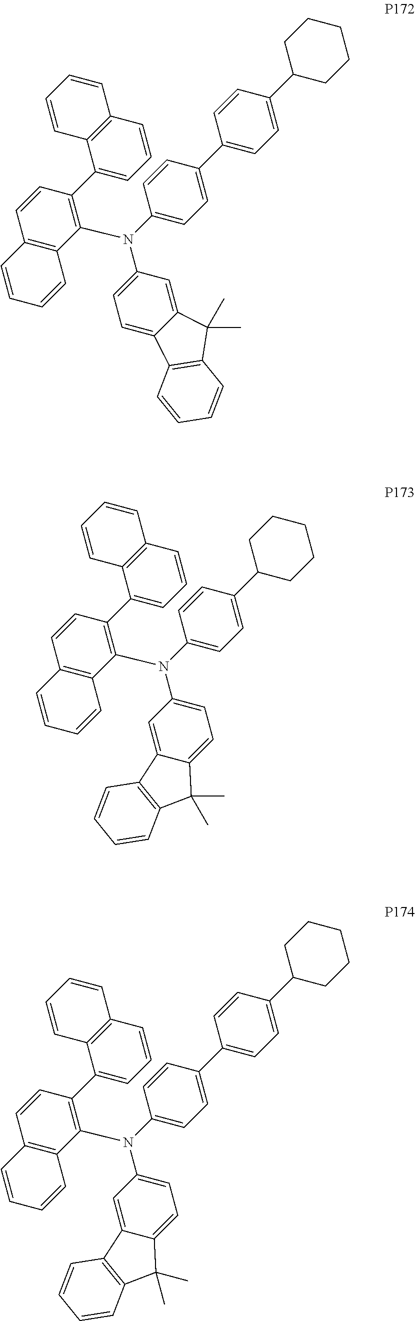 [SMILES: CC1(C)c2ccccc2-c2ccc(N(c3ccc(-c4ccc(C5CCCCC5)cc4)cc3)c3c(-c4cccc5ccccc45)ccc4ccccc34)cc21 ; CC1(C)c2ccccc2-c2cc(N(c3ccc(C4CCCCC4)cc3)c3c(-c4cccc5ccccc45)ccc4ccccc34)ccc21 ; CC1(C)c2ccccc2-c2cc(N(c3ccc(-c4ccc(C5CCCCC5)cc4)cc3)c3c(-c4cccc5ccccc45)ccc4ccccc34)ccc21]

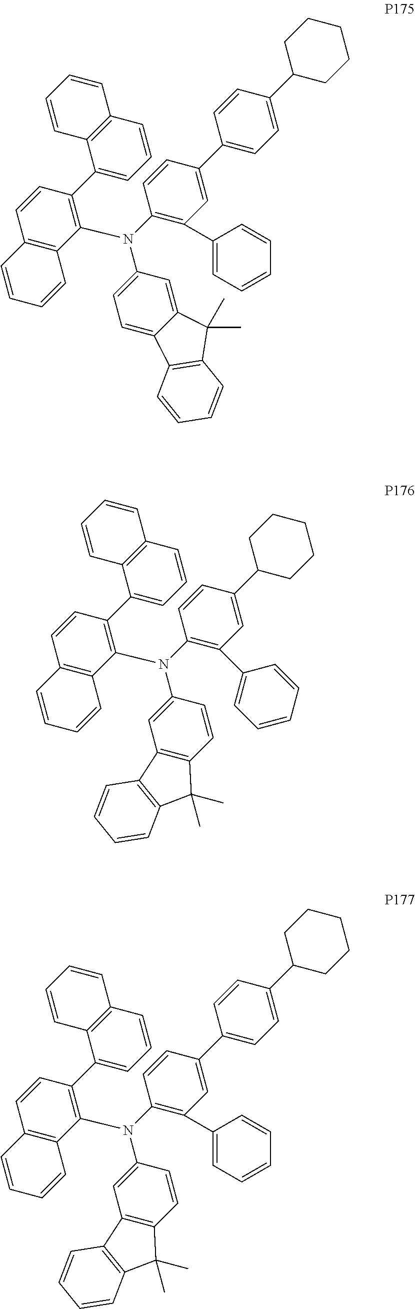 [SMILES: CC1(C)c2ccccc2-c2ccc(N(c3ccc(-c4ccc(C5CCCCC5)cc4)cc3-c3ccccc3)c3c(-c4cccc5ccccc45)ccc4ccccc34)cc21 ; CC1(C)c2ccccc2-c2cc(N(c3ccc(C4CCCCC4)cc3-c3ccccc3)c3c(-c4cccc5ccccc45)ccc4ccccc34)ccc21 ; CC1(C)c2ccccc2-c2cc(N(c3ccc(-c4ccc(C5CCCCC5)cc4)cc3-c3ccccc3)c3c(-c4cccc5ccccc45)ccc4ccccc34)ccc21]

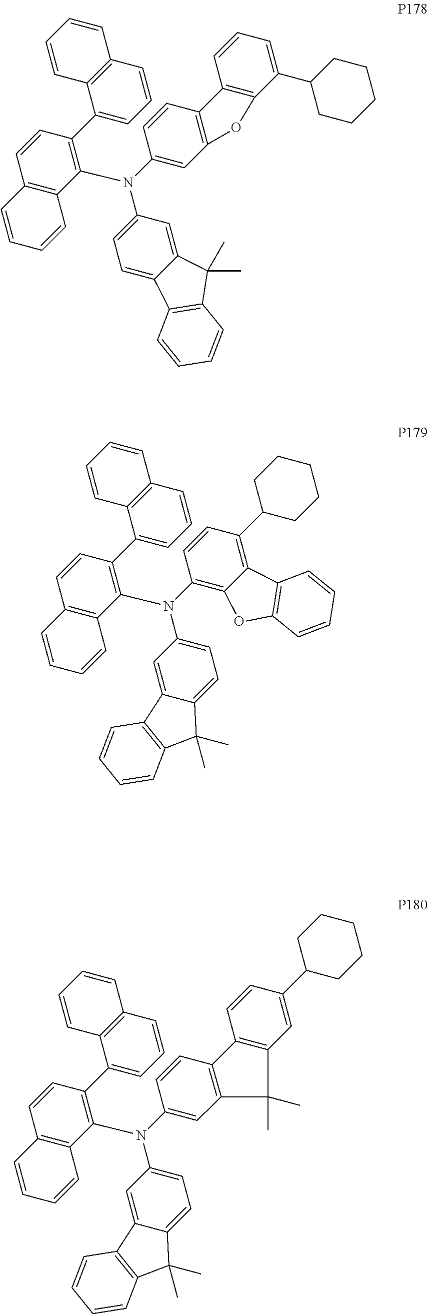 [SMILES: CC1(C)c2ccccc2-c2ccc(N(c3ccc4c(c3)oc3c(C5CCCCC5)cccc34)c3c(-c4cccc5ccccc45)ccc4ccccc34)cc21 ; CC1(C)c2ccccc2-c2cc(N(c3c(-c4cccc5ccccc45)ccc4ccccc34)c3ccc(C4CCCCC4)c4c3oc3ccccc34)ccc21 ; CC1(C)c2ccccc2-c2cc(N(c3ccc4c(c3)C(C)(C)c3cc(C5CCCCC5)ccc3-4)c3c(-c4cccc5ccccc45)ccc4ccccc34)ccc21]

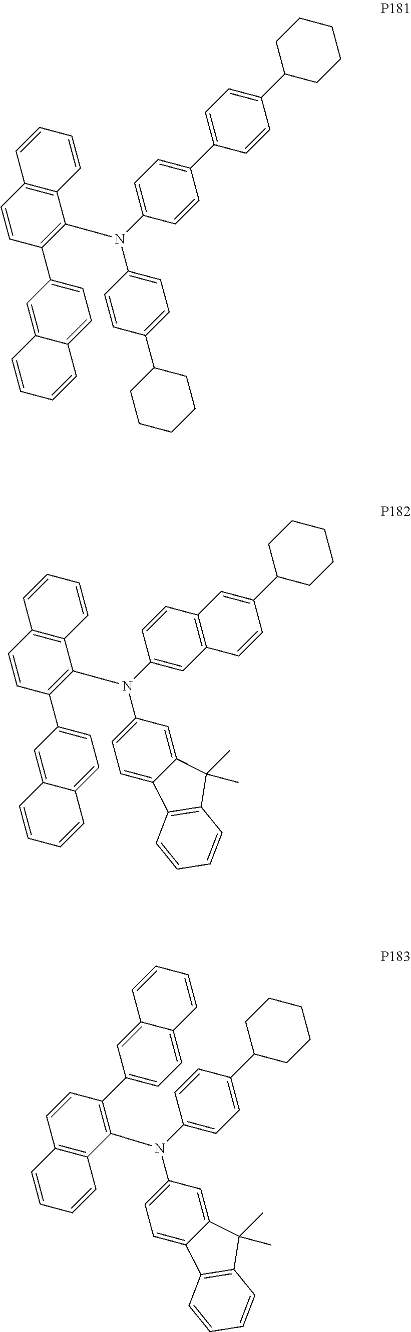 [SMILES: c1ccc2cc(-c3ccc4ccccc4c3N(c3ccc(-c4ccc(C5CCCCC5)cc4)cc3)c3ccc(C4CCCCC4)cc3)ccc2c1 ; CC1(C)c2ccccc2-c2ccc(N(c3ccc4cc(C5CCCCC5)ccc4c3)c3c(-c4ccc5ccccc5c4)ccc4ccccc34)cc21 ; CC1(C)c2ccccc2-c2ccc(N(c3ccc(C4CCCCC4)cc3)c3c(-c4ccc5ccccc5c4)ccc4ccccc34)cc21]

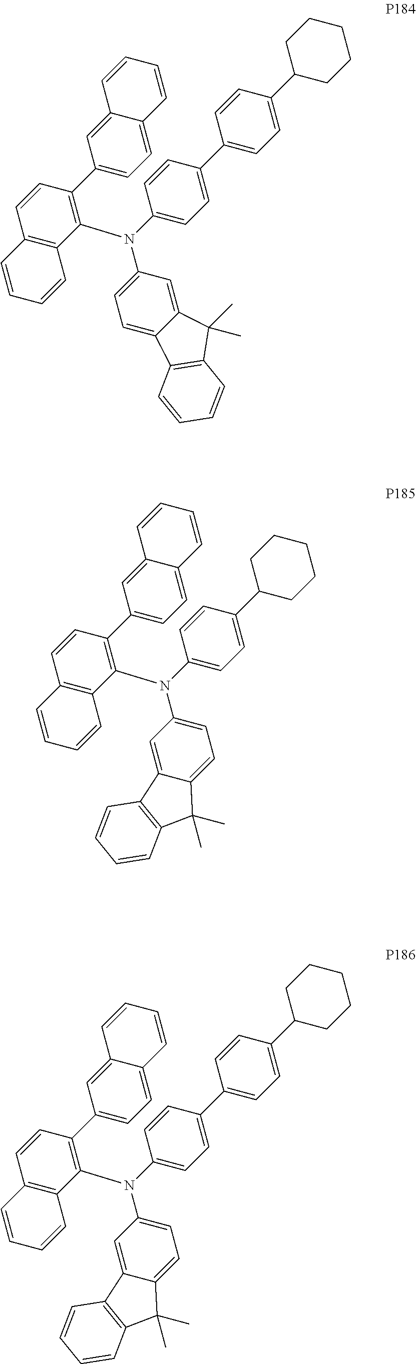 [SMILES: CC1(C)c2ccccc2-c2ccc(N(c3ccc(-c4ccc(C5CCCCC5)cc4)cc3)c3c(-c4ccc5ccccc5c4)ccc4ccccc34)cc21 ; CC1(C)c2ccccc2-c2cc(N(c3ccc(C4CCCCC4)cc3)c3c(-c4ccc5ccccc5c4)ccc4ccccc34)ccc21 ; CC1(C)c2ccccc2-c2cc(N(c3ccc(-c4ccc(C5CCCCC5)cc4)cc3)c3c(-c4ccc5ccccc5c4)ccc4ccccc34)ccc21]

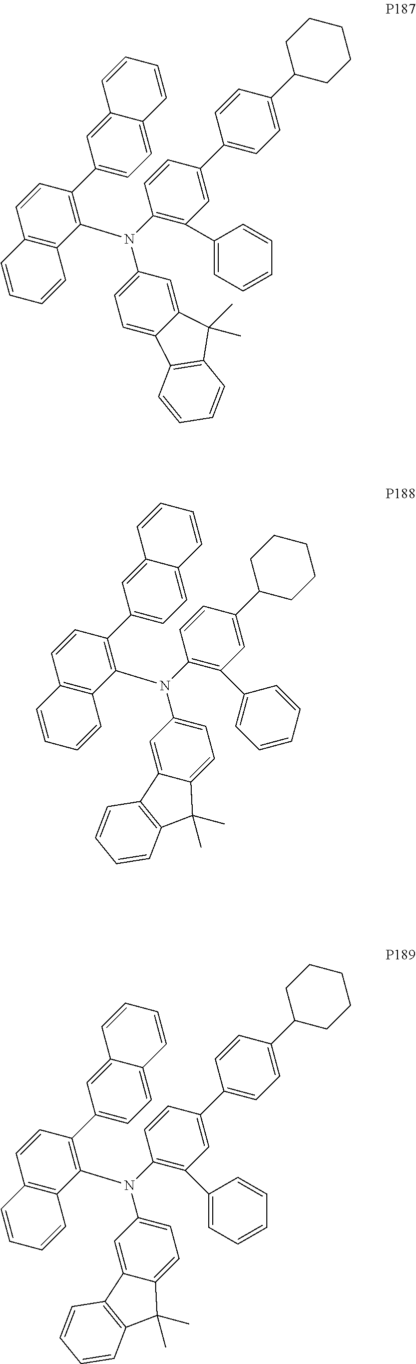 [SMILES: CC1(C)c2ccccc2-c2ccc(N(c3ccc(-c4ccc(C5CCCCC5)cc4)cc3-c3ccccc3)c3c(-c4ccc5ccccc5c4)ccc4ccccc34)cc21 ; CC1(C)c2ccccc2-c2cc(N(c3ccc(C4CCCCC4)cc3-c3ccccc3)c3c(-c4ccc5ccccc5c4)ccc4ccccc34)ccc21 ; CC1(C)c2ccccc2-c2cc(N(c3ccc(-c4ccc(C5CCCCC5)cc4)cc3-c3ccccc3)c3c(-c4ccc5ccccc5c4)ccc4ccccc34)ccc21]

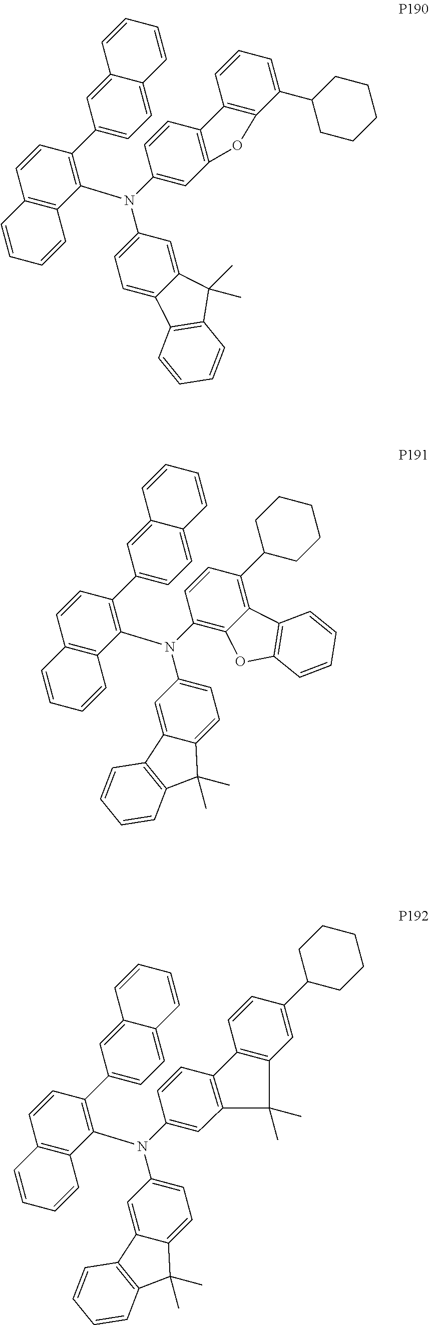 [SMILES: CC1(C)c2ccccc2-c2ccc(N(c3ccc4c(c3)oc3c(C5CCCCC5)cccc34)c3c(-c4ccc5ccccc5c4)ccc4ccccc34)cc21 ; CC1(C)c2ccccc2-c2cc(N(c3c(-c4ccc5ccccc5c4)ccc4ccccc34)c3ccc(C4CCCCC4)c4c3oc3ccccc34)ccc21 ; CC1(C)c2ccccc2-c2cc(N(c3ccc4c(c3)C(C)(C)c3cc(C5CCCCC5)ccc3-4)c3c(-c4ccc5ccccc5c4)ccc4ccccc34)ccc21]

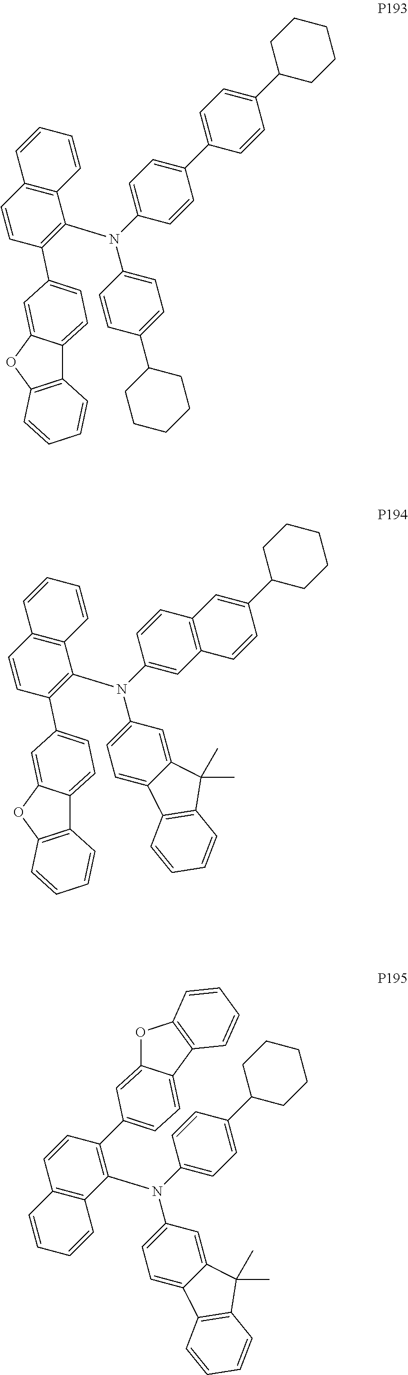 [SMILES: c1ccc2c(N(c3ccc(-c4ccc(C5CCCCC5)cc4)cc3)c3ccc(C4CCCCC4)cc3)c(-c3ccc4c(c3)oc3ccccc34)ccc2c1 ; CC1(C)c2ccccc2-c2ccc(N(c3ccc4cc(C5CCCCC5)ccc4c3)c3c(-c4ccc5c(c4)oc4ccccc45)ccc4ccccc34)cc21 ; CC1(C)c2ccccc2-c2ccc(N(c3ccc(C4CCCCC4)cc3)c3c(-c4ccc5c(c4)oc4ccccc45)ccc4ccccc34)cc21]

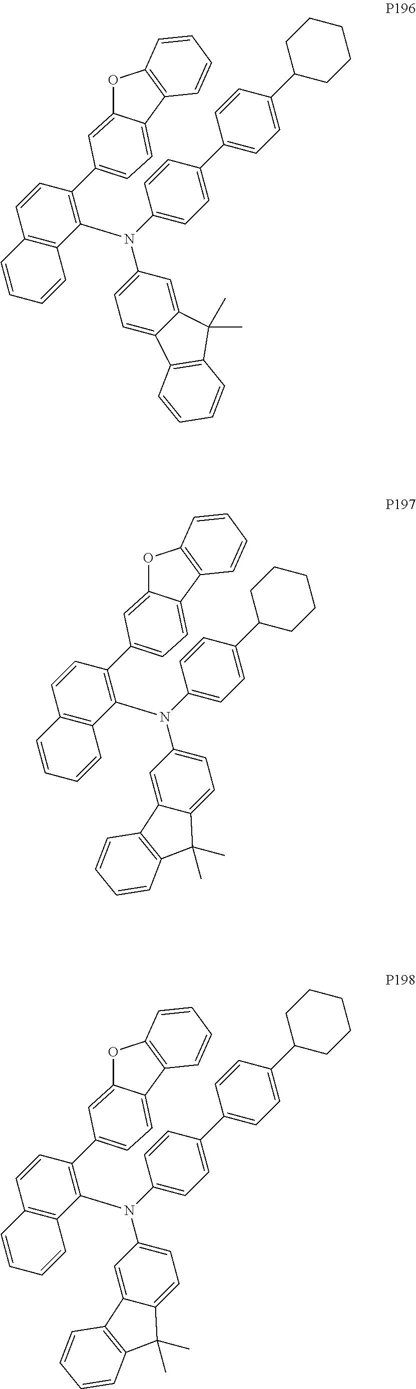 [SMILES: CC1(C)c2ccccc2-c2ccc(N(c3ccc(-c4ccc(C5CCCCC5)cc4)cc3)c3c(-c4ccc5c(c4)oc4ccccc45)ccc4ccccc34)cc21 ; CC1(C)c2ccccc2-c2cc(N(c3ccc(C4CCCCC4)cc3)c3c(-c4ccc5c(c4)oc4ccccc45)ccc4ccccc34)ccc21 ; CC1(C)c2ccccc2-c2cc(N(c3ccc(-c4ccc(C5CCCCC5)cc4)cc3)c3c(-c4ccc5c(c4)oc4ccccc45)ccc4ccccc34)ccc21]

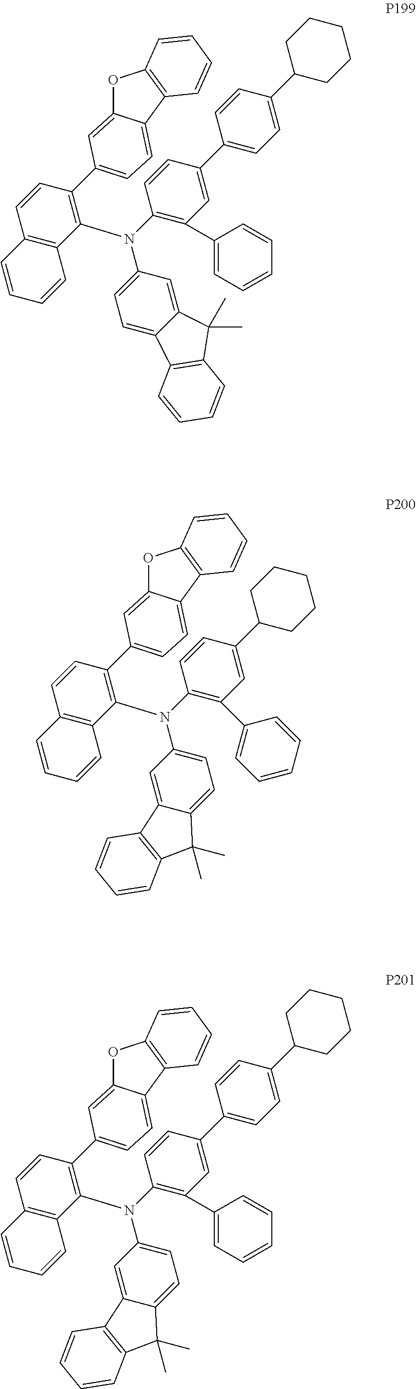 [SMILES: CC1(C)c2ccccc2-c2ccc(N(c3ccc(-c4ccc(C5CCCCC5)cc4)cc3-c3ccccc3)c3c(-c4ccc5c(c4)oc4ccccc45)ccc4ccccc34)cc21 ; CC1(C)c2ccccc2-c2cc(N(c3ccc(C4CCCCC4)cc3-c3ccccc3)c3c(-c4ccc5c(c4)oc4ccccc45)ccc4ccccc34)ccc21 ; CC1(C)c2ccccc2-c2cc(N(c3ccc(-c4ccc(C5CCCCC5)cc4)cc3-c3ccccc3)c3c(-c4ccc5c(c4)oc4ccccc45)ccc4ccccc34)ccc21]

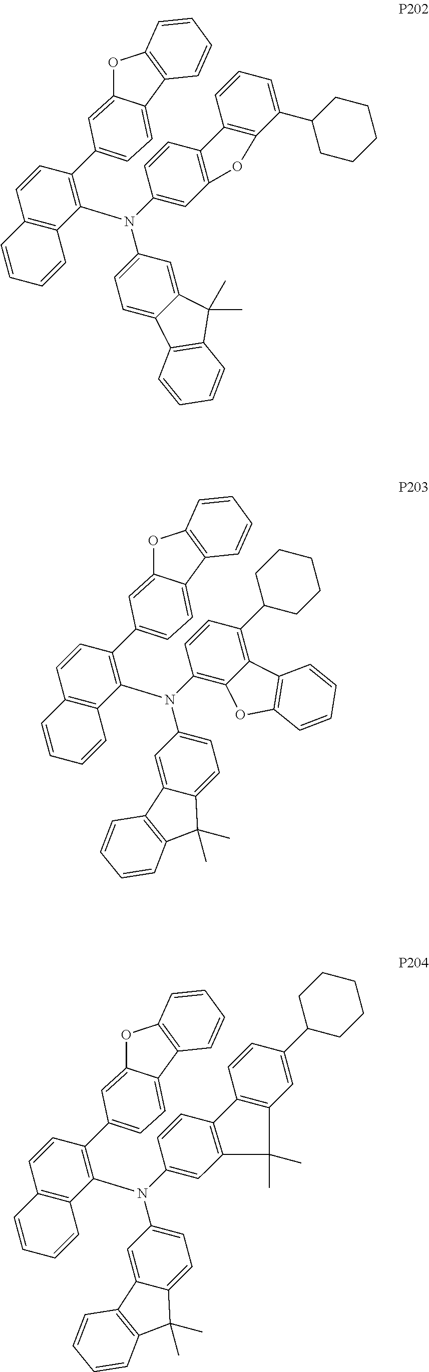 [SMILES: CC1(C)c2ccccc2-c2ccc(N(c3ccc4c(c3)oc3c(C5CCCCC5)cccc34)c3c(-c4ccc5c(c4)oc4ccccc45)ccc4ccccc34)cc21 ; CC1(C)c2ccccc2-c2cc(N(c3c(-c4ccc5c(c4)oc4ccccc45)ccc4ccccc34)c3ccc(C4CCCCC4)c4c3oc3ccccc34)ccc21 ; CC1(C)c2ccccc2-c2cc(N(c3ccc4c(c3)C(C)(C)c3cc(C5CCCCC5)ccc3-4)c3c(-c4ccc5c(c4)oc4ccccc45)ccc4ccccc34)ccc21]

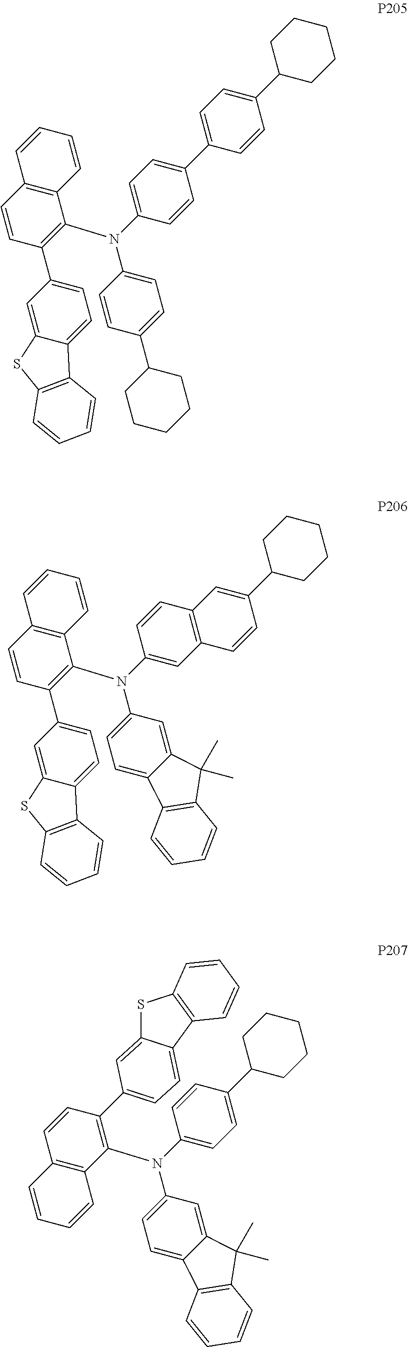 [SMILES: c1ccc2c(N(c3ccc(-c4ccc(C5CCCCC5)cc4)cc3)c3ccc(C4CCCCC4)cc3)c(-c3ccc4c(c3)sc3ccccc34)ccc2c1 ; CC1(C)c2ccccc2-c2ccc(N(c3ccc4cc(C5CCCCC5)ccc4c3)c3c(-c4ccc5c(c4)sc4ccccc45)ccc4ccccc34)cc21 ; CC1(C)c2ccccc2-c2ccc(N(c3ccc(C4CCCCC4)cc3)c3c(-c4ccc5c(c4)sc4ccccc45)ccc4ccccc34)cc21]

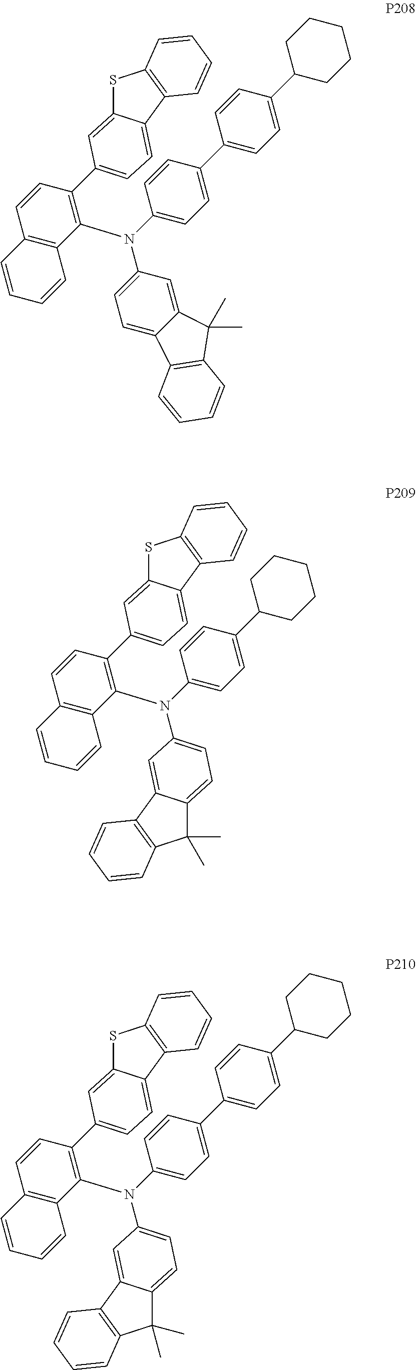 [SMILES: CC1(C)c2ccccc2-c2ccc(N(c3ccc(-c4ccc(C5CCCCC5)cc4)cc3)c3c(-c4ccc5c(c4)sc4ccccc45)ccc4ccccc34)cc21 ; CC1(C)c2ccccc2-c2cc(N(c3ccc(C4CCCCC4)cc3)c3c(-c4ccc5c(c4)sc4ccccc45)ccc4ccccc34)ccc21 ; CC1(C)c2ccccc2-c2cc(N(c3ccc(-c4ccc(C5CCCCC5)cc4)cc3)c3c(-c4ccc5c(c4)sc4ccccc45)ccc4ccccc34)ccc21]

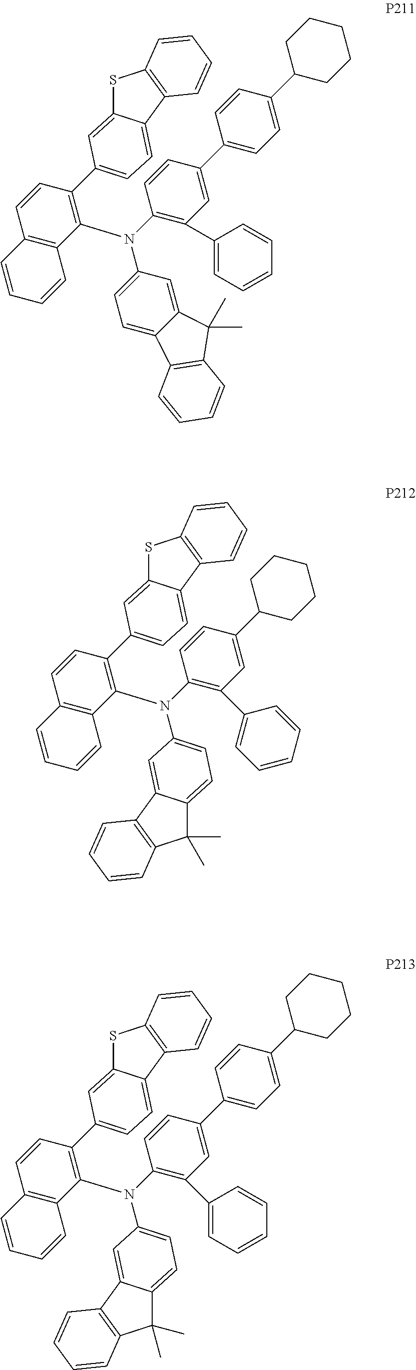 [SMILES: CC1(C)c2ccccc2-c2ccc(N(c3ccc(-c4ccc(C5CCCCC5)cc4)cc3-c3ccccc3)c3c(-c4ccc5c(c4)sc4ccccc45)ccc4ccccc34)cc21 ; CC1(C)c2ccccc2-c2cc(N(c3ccc(C4CCCCC4)cc3-c3ccccc3)c3c(-c4ccc5c(c4)sc4ccccc45)ccc4ccccc34)ccc21 ; CC1(C)c2ccccc2-c2cc(N(c3ccc(-c4ccc(C5CCCCC5)cc4)cc3-c3ccccc3)c3c(-c4ccc5c(c4)sc4ccccc45)ccc4ccccc34)ccc21]

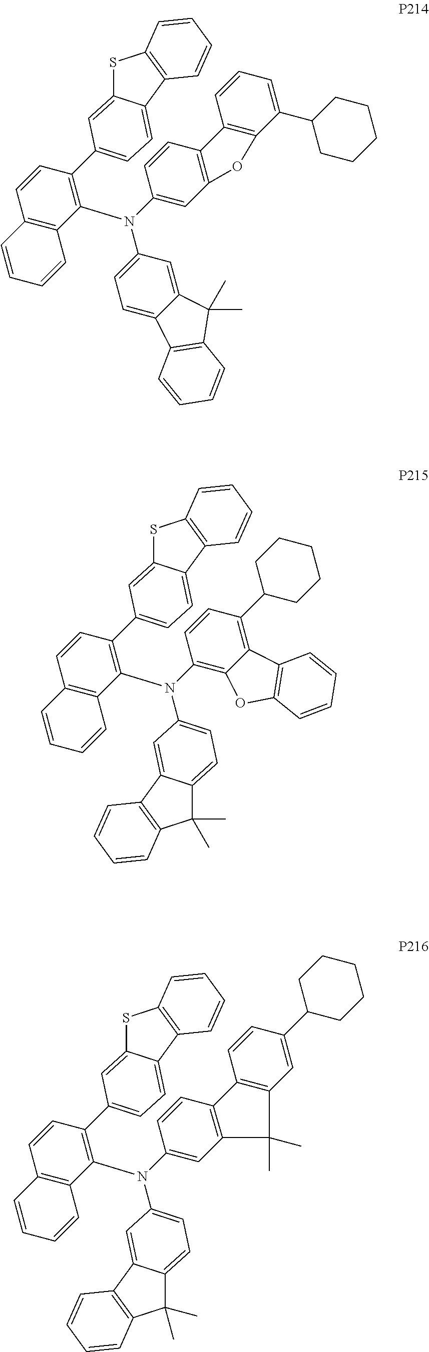 [SMILES: CC1(C)c2ccccc2-c2ccc(N(c3ccc4c(c3)oc3c(C5CCCCC5)cccc34)c3c(-c4ccc5c(c4)sc4ccccc45)ccc4ccccc34)cc21 ; CC1(C)c2ccccc2-c2cc(N(c3c(-c4ccc5c(c4)sc4ccccc45)ccc4ccccc34)c3ccc(C4CCCCC4)c4c3oc3ccccc34)ccc21 ; CC1(C)c2ccccc2-c2cc(N(c3ccc4c(c3)C(C)(C)c3cc(C5CCCCC5)ccc3-4)c3c(-c4ccc5c(c4)sc4ccccc45)ccc4ccccc34)ccc21]

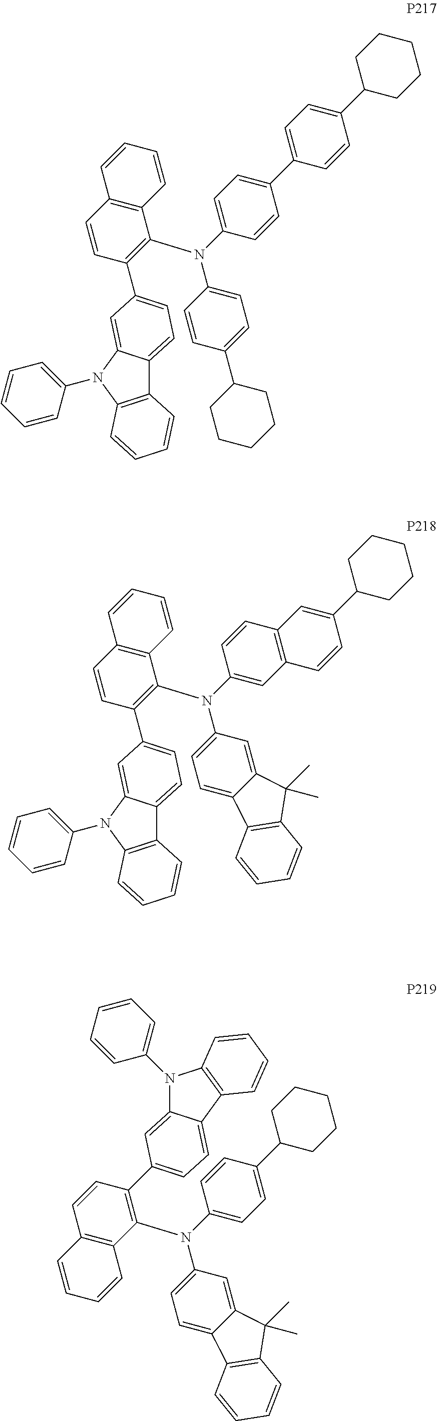 [SMILES: c1ccc(-n2c3ccccc3c3ccc(-c4ccc5ccccc5c4N(c4ccc(-c5ccc(C6CCCCC6)cc5)cc4)c4ccc(C5CCCCC5)cc4)cc32)cc1 ; CC1(C)c2ccccc2-c2ccc(N(c3ccc4cc(C5CCCCC5)ccc4c3)c3c(-c4ccc5c6ccccc6n(-c6ccccc6)c5c4)ccc4ccccc34)cc21 ; CC1(C)c2ccccc2-c2ccc(N(c3ccc(C4CCCCC4)cc3)c3c(-c4ccc5c6ccccc6n(-c6ccccc6)c5c4)ccc4ccccc34)cc21]

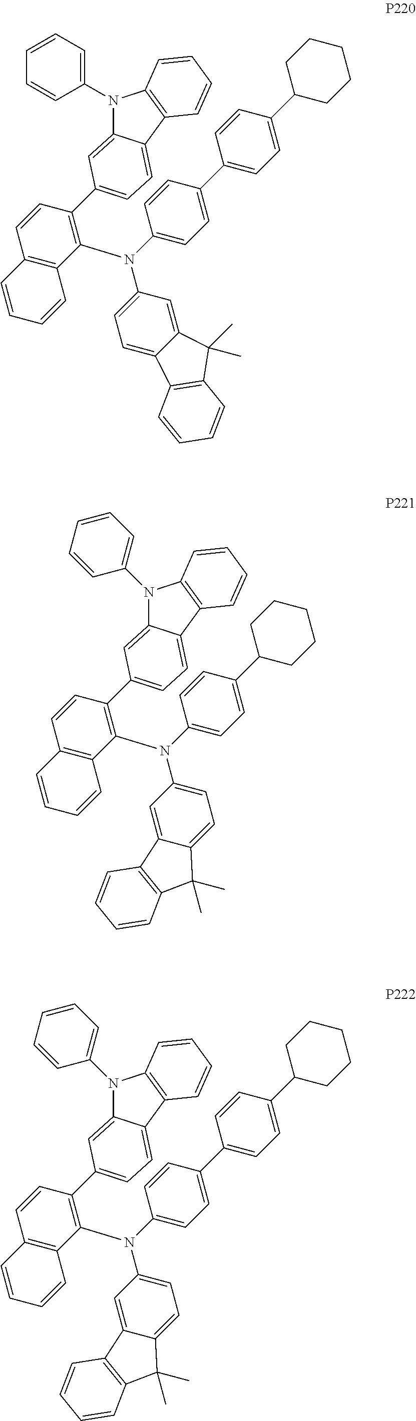 [SMILES: CC1(C)c2ccccc2-c2ccc(N(c3ccc(-c4ccc(C5CCCCC5)cc4)cc3)c3c(-c4ccc5c6ccccc6n(-c6ccccc6)c5c4)ccc4ccccc34)cc21 ; CC1(C)c2ccccc2-c2cc(N(c3ccc(C4CCCCC4)cc3)c3c(-c4ccc5c6ccccc6n(-c6ccccc6)c5c4)ccc4ccccc34)ccc21 ; CC1(C)c2ccccc2-c2cc(N(c3ccc(-c4ccc(C5CCCCC5)cc4)cc3)c3c(-c4ccc5c6ccccc6n(-c6ccccc6)c5c4)ccc4ccccc34)ccc21]

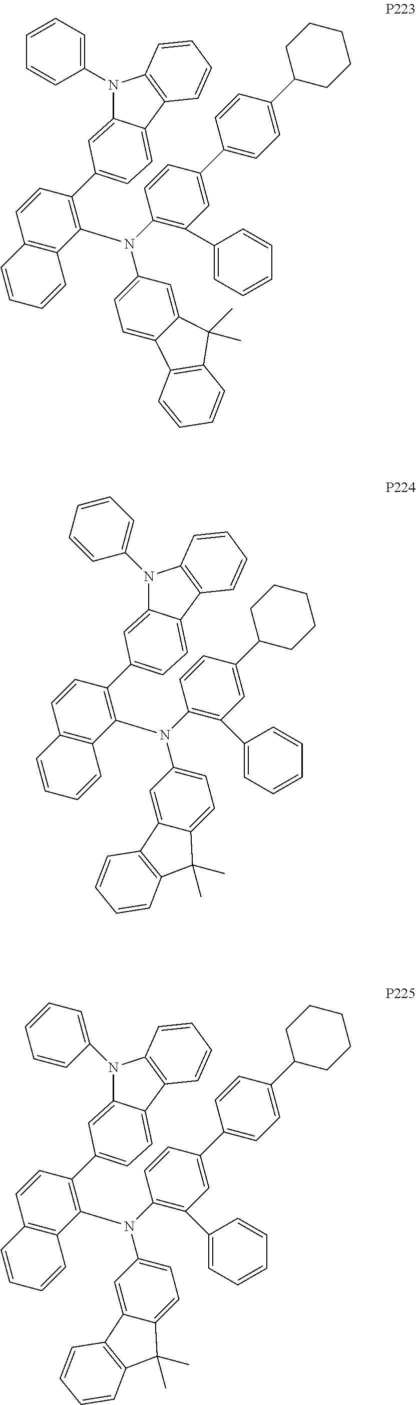 [SMILES: CC1(C)c2ccccc2-c2ccc(N(c3ccc(-c4ccc(C5CCCCC5)cc4)cc3-c3ccccc3)c3c(-c4ccc5c6ccccc6n(-c6ccccc6)c5c4)ccc4ccccc34)cc21 ; CC1(C)c2ccccc2-c2cc(N(c3ccc(C4CCCCC4)cc3-c3ccccc3)c3c(-c4ccc5c6ccccc6n(-c6ccccc6)c5c4)ccc4ccccc34)ccc21 ; CC1(C)c2ccccc2-c2cc(N(c3ccc(-c4ccc(C5CCCCC5)cc4)cc3-c3ccccc3)c3c(-c4ccc5c6ccccc6n(-c6ccccc6)c5c4)ccc4ccccc34)ccc21]

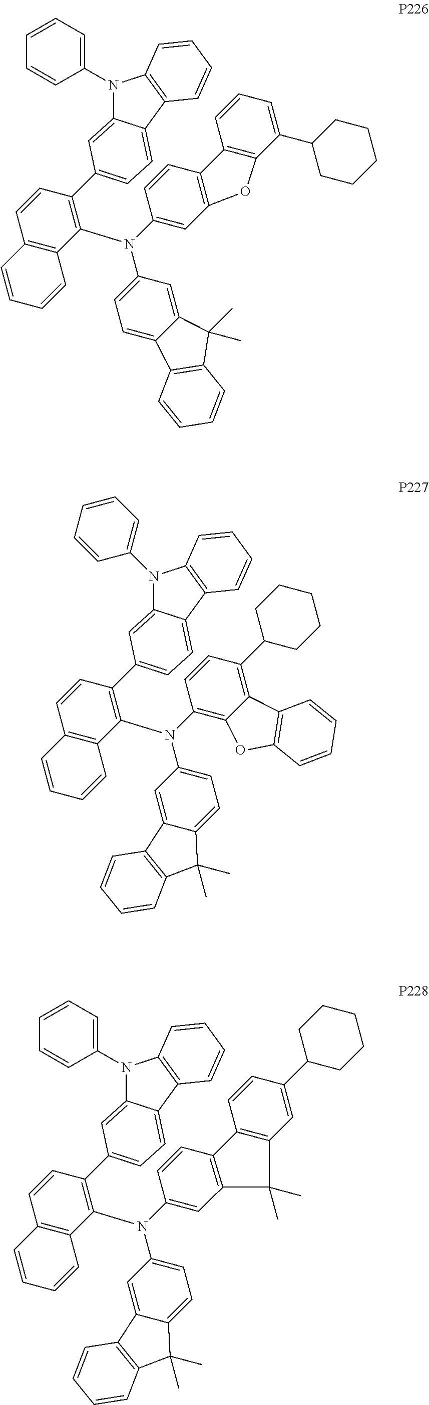 [SMILES: CC1(C)c2ccccc2-c2ccc(N(c3ccc4c(c3)oc3c(C5CCCCC5)cccc34)c3c(-c4ccc5c6ccccc6n(-c6ccccc6)c5c4)ccc4ccccc34)cc21 ; CC1(C)c2ccccc2-c2cc(N(c3c(-c4ccc5c6ccccc6n(-c6ccccc6)c5c4)ccc4ccccc34)c3ccc(C4CCCCC4)c4c3oc3ccccc34)ccc21 ; CC1(C)c2ccccc2-c2cc(N(c3ccc4c(c3)C(C)(C)c3cc(C5CCCCC5)ccc3-4)c3c(-c4ccc5c6ccccc6n(-c6ccccc6)c5c4)ccc4ccccc34)ccc21]

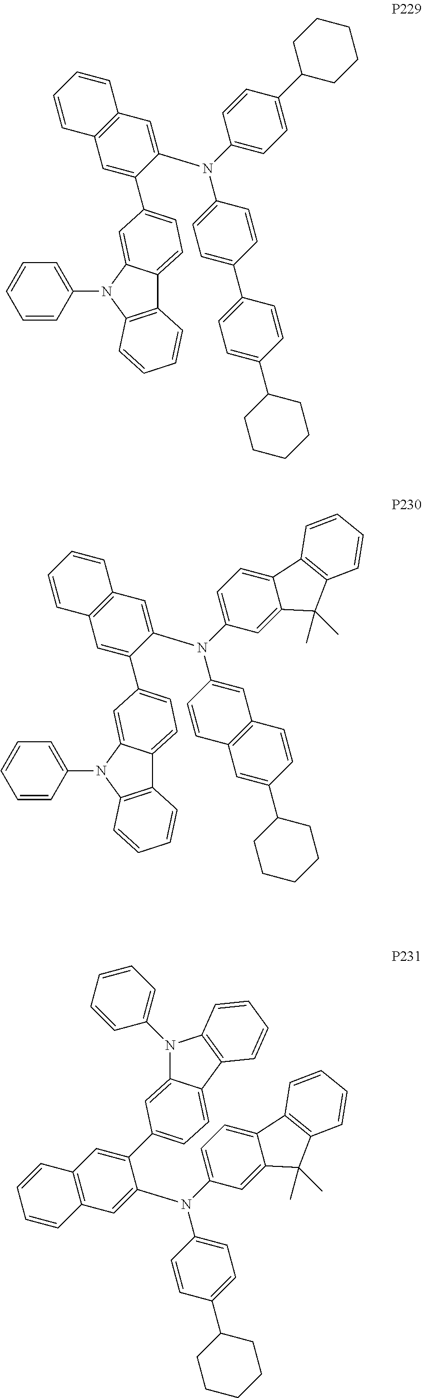 [SMILES: c1ccc(-n2c3ccccc3c3ccc(-c4cc5ccccc5cc4N(c4ccc(-c5ccc(C6CCCCC6)cc5)cc4)c4ccc(C5CCCCC5)cc4)cc32)cc1 ; CC1(C)c2ccccc2-c2ccc(N(c3ccc4cc(C5CCCCC5)ccc4c3)c3cc4ccccc4cc3-c3ccc4c5ccccc5n(-c5ccccc5)c4c3)cc21 ; CC1(C)c2ccccc2-c2ccc(N(c3ccc(C4CCCCC4)cc3)c3cc4ccccc4cc3-c3ccc4c5ccccc5n(-c5ccccc5)c4c3)cc21]

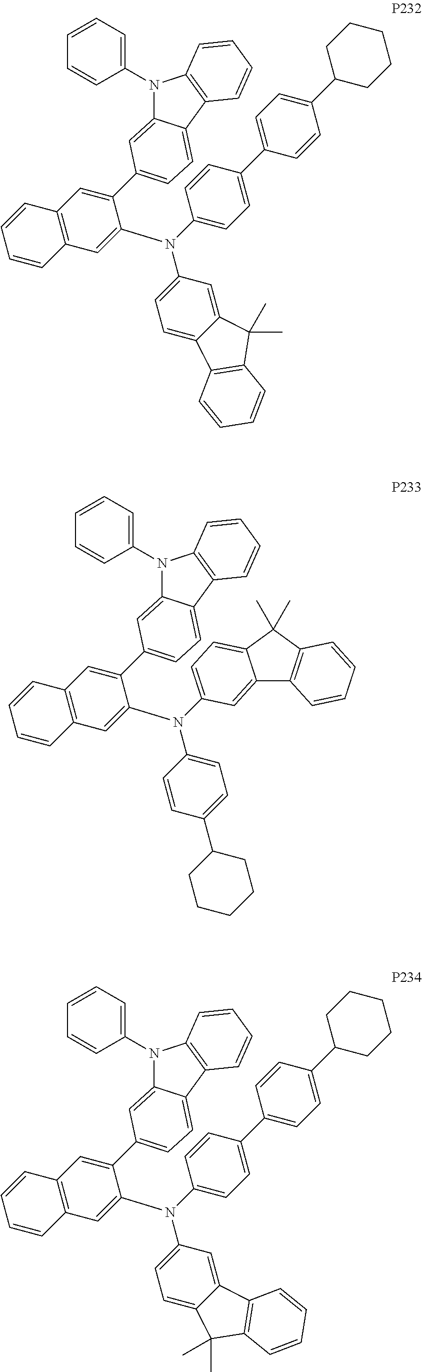 [SMILES: CC1(C)c2ccccc2-c2ccc(N(c3ccc(-c4ccc(C5CCCCC5)cc4)cc3)c3cc4ccccc4cc3-c3ccc4c5ccccc5n(-c5ccccc5)c4c3)cc21 ; CC1(C)c2ccccc2-c2cc(N(c3ccc(C4CCCCC4)cc3)c3cc4ccccc4cc3-c3ccc4c5ccccc5n(-c5ccccc5)c4c3)ccc21 ; CC1(C)c2ccccc2-c2cc(N(c3ccc(-c4ccc(C5CCCCC5)cc4)cc3)c3cc4ccccc4cc3-c3ccc4c5ccccc5n(-c5ccccc5)c4c3)ccc21]

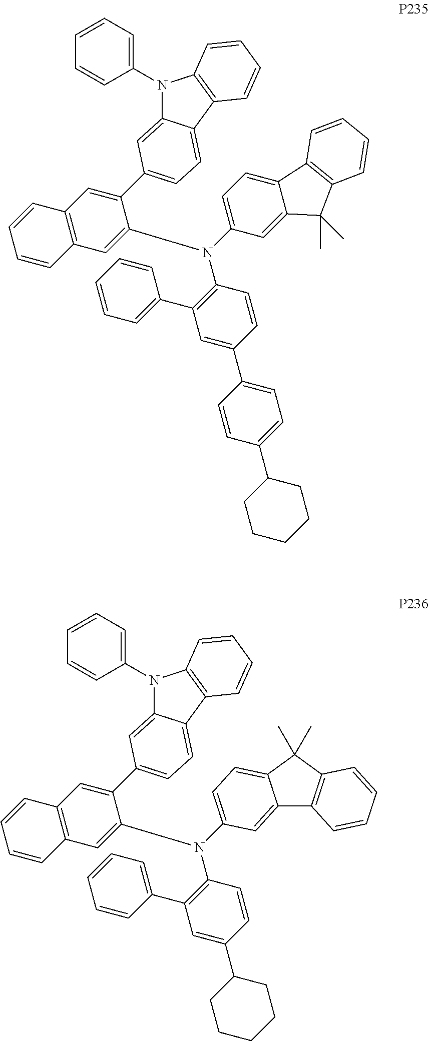 [SMILES: CC1(C)c2ccccc2-c2ccc(N(c3ccc(-c4ccc(C5CCCCC5)cc4)cc3-c3ccccc3)c3cc4ccccc4cc3-c3ccc4c5ccccc5n(-c5ccccc5)c4c3)cc21 ; CC1(C)c2ccccc2-c2cc(N(c3ccc(C4CCCCC4)cc3-c3ccccc3)c3cc4ccccc4cc3-c3ccc4c5ccccc5n(-c5ccccc5)c4c3)ccc21]

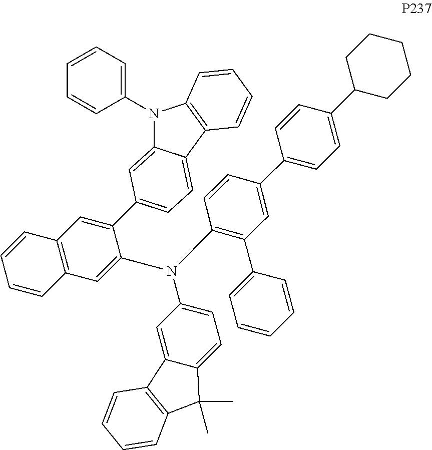 [SMILES: CC1(C)c2ccccc2-c2cc(N(c3ccc(-c4ccc(C5CCCCC5)cc4)cc3-c3ccccc3)c3cc4ccccc4cc3-c3ccc4c5ccccc5n(-c5ccccc5)c4c3)ccc21]

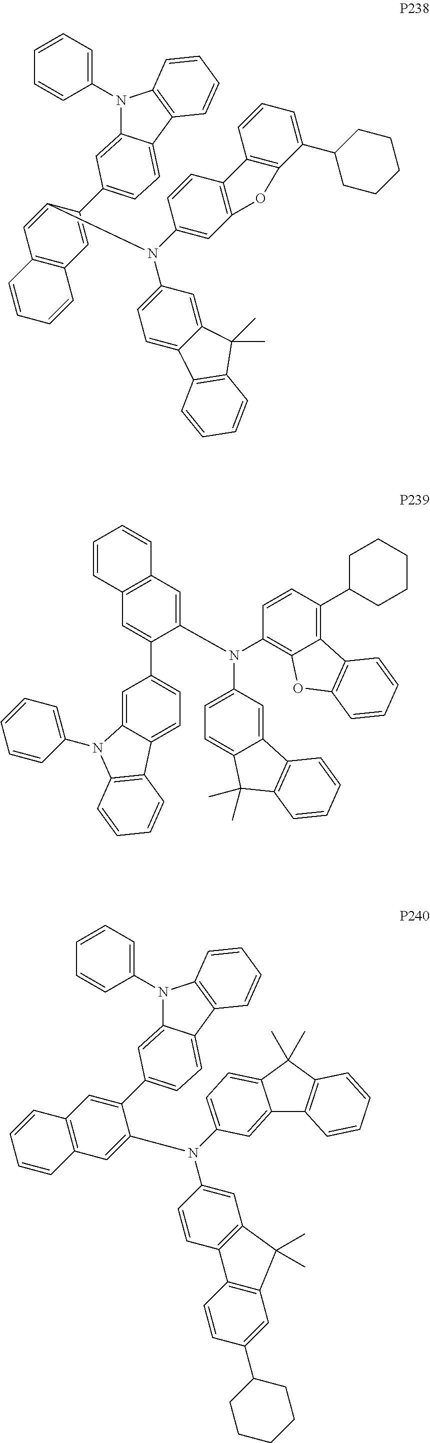 [SMILES: CC1(C)c2ccccc2-c2ccc(N(c3ccc4c(c3)oc3c(C5CCCCC5)cccc34)c3cc4ccccc4cc3-c3ccc4c5ccccc5n(-c5ccccc5)c4c3)cc21 ; CC1(C)c2ccccc2-c2cc(N(c3cc4ccccc4cc3-c3ccc4c5ccccc5n(-c5ccccc5)c4c3)c3ccc(C4CCCCC4)c4c3oc3ccccc34)ccc21 ; CC1(C)c2ccccc2-c2cc(N(c3ccc4c(c3)C(C)(C)c3cc(C5CCCCC5)ccc3-4)c3cc4ccccc4cc3-c3ccc4c5ccccc5n(-c5ccccc5)c4c3)ccc21]

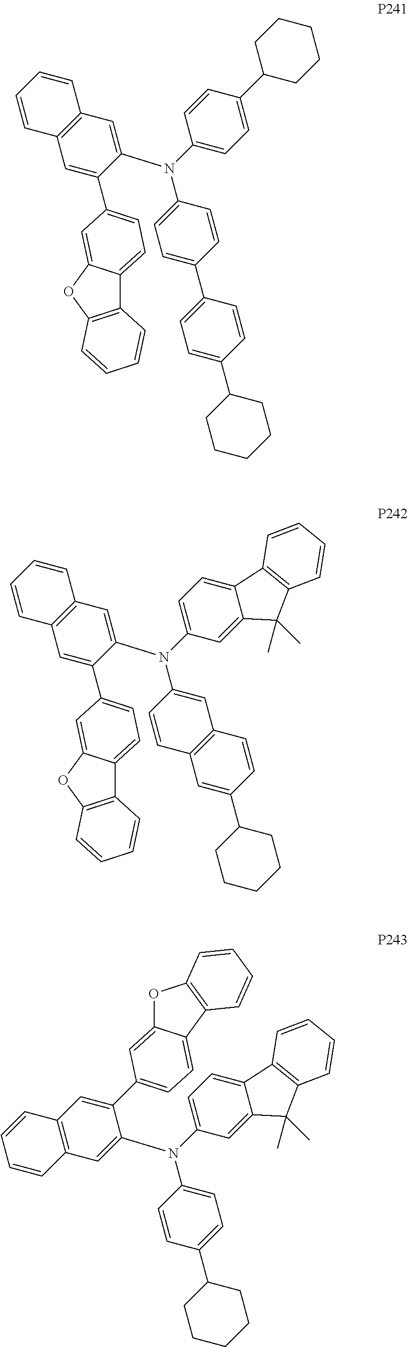 [SMILES: c1ccc2cc(N(c3ccc(-c4ccc(C5CCCCC5)cc4)cc3)c3ccc(C4CCCCC4)cc3)c(-c3ccc4c(c3)oc3ccccc34)cc2c1 ; CC1(C)c2ccccc2-c2ccc(N(c3ccc4cc(C5CCCCC5)ccc4c3)c3cc4ccccc4cc3-c3ccc4c(c3)oc3ccccc34)cc21 ; CC1(C)c2ccccc2-c2ccc(N(c3ccc(C4CCCCC4)cc3)c3cc4ccccc4cc3-c3ccc4c(c3)oc3ccccc34)cc21]

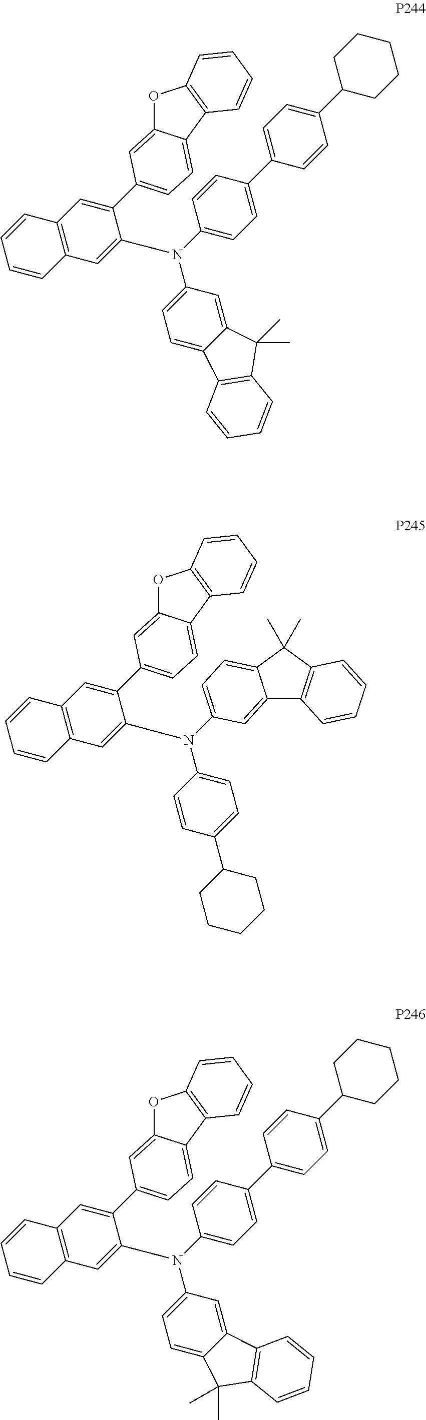 [SMILES: CC1(C)c2ccccc2-c2ccc(N(c3ccc(-c4ccc(C5CCCCC5)cc4)cc3)c3cc4ccccc4cc3-c3ccc4c(c3)oc3ccccc34)cc21 ; CC1(C)c2ccccc2-c2cc(N(c3ccc(C4CCCCC4)cc3)c3cc4ccccc4cc3-c3ccc4c(c3)oc3ccccc34)ccc21 ; CC1(C)c2ccccc2-c2cc(N(c3ccc(-c4ccc(C5CCCCC5)cc4)cc3)c3cc4ccccc4cc3-c3ccc4c(c3)oc3ccccc34)ccc21]

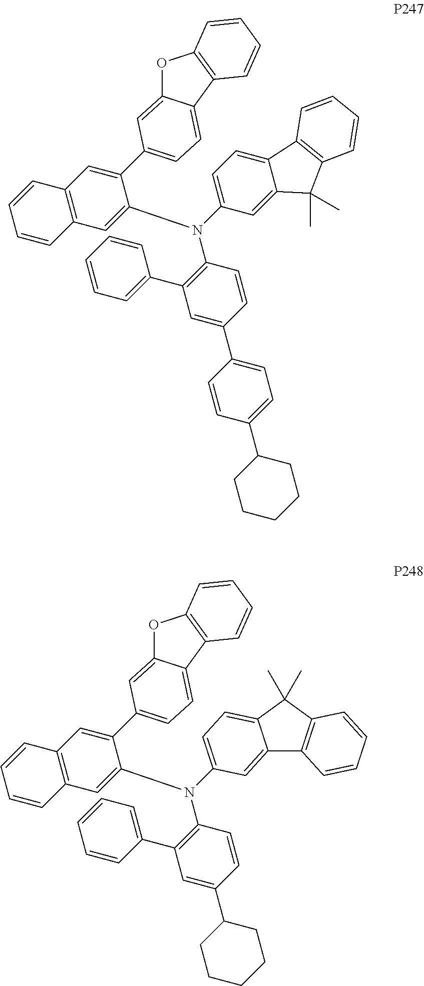 [SMILES: CC1(C)c2ccccc2-c2ccc(N(c3ccc(-c4ccc(C5CCCCC5)cc4)cc3-c3ccccc3)c3cc4ccccc4cc3-c3ccc4c(c3)oc3ccccc34)cc21 ; CC1(C)c2ccccc2-c2cc(N(c3ccc(C4CCCCC4)cc3-c3ccccc3)c3cc4ccccc4cc3-c3ccc4c(c3)oc3ccccc34)ccc21]

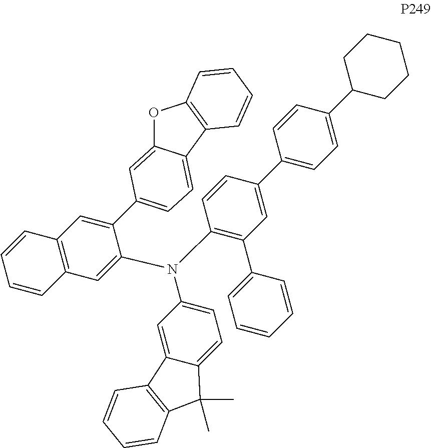 [SMILES: CC1(C)c2ccccc2-c2cc(N(c3ccc(-c4ccc(C5CCCCC5)cc4)cc3-c3ccccc3)c3cc4ccccc4cc3-c3ccc4c(c3)oc3ccccc34)ccc21]

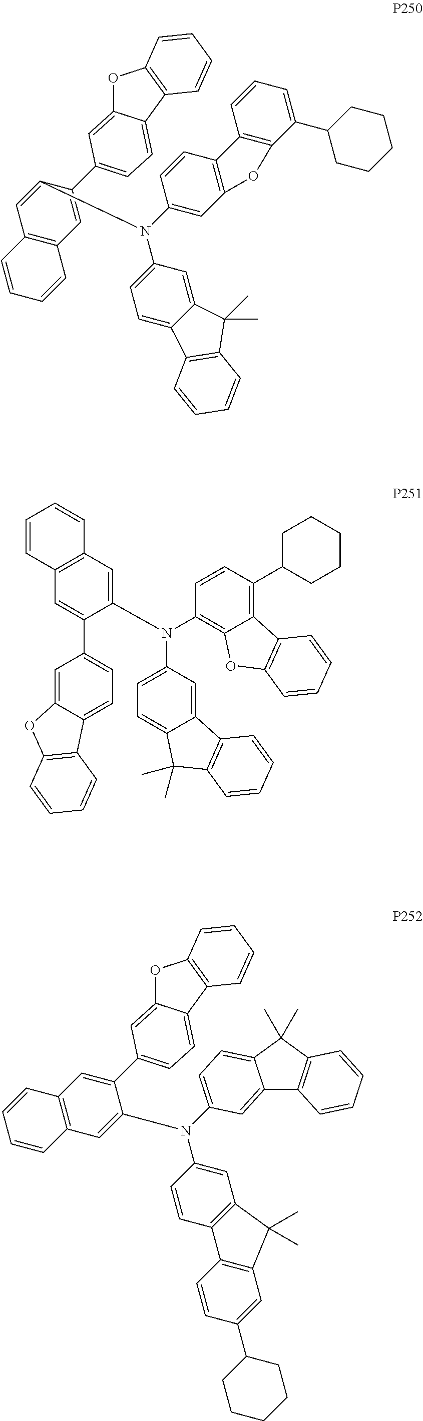 [SMILES: CC1(C)c2ccccc2-c2ccc(N(c3ccc4c(c3)oc3c(C5CCCCC5)cccc34)c3cc4ccccc4cc3-c3ccc4c(c3)oc3ccccc34)cc21 ; CC1(C)c2ccccc2-c2cc(N(c3cc4ccccc4cc3-c3ccc4c(c3)oc3ccccc34)c3ccc(C4CCCCC4)c4c3oc3ccccc34)ccc21 ; CC1(C)c2ccccc2-c2cc(N(c3ccc4c(c3)C(C)(C)c3cc(C5CCCCC5)ccc3-4)c3cc4ccccc4cc3-c3ccc4c(c3)oc3ccccc34)ccc21]

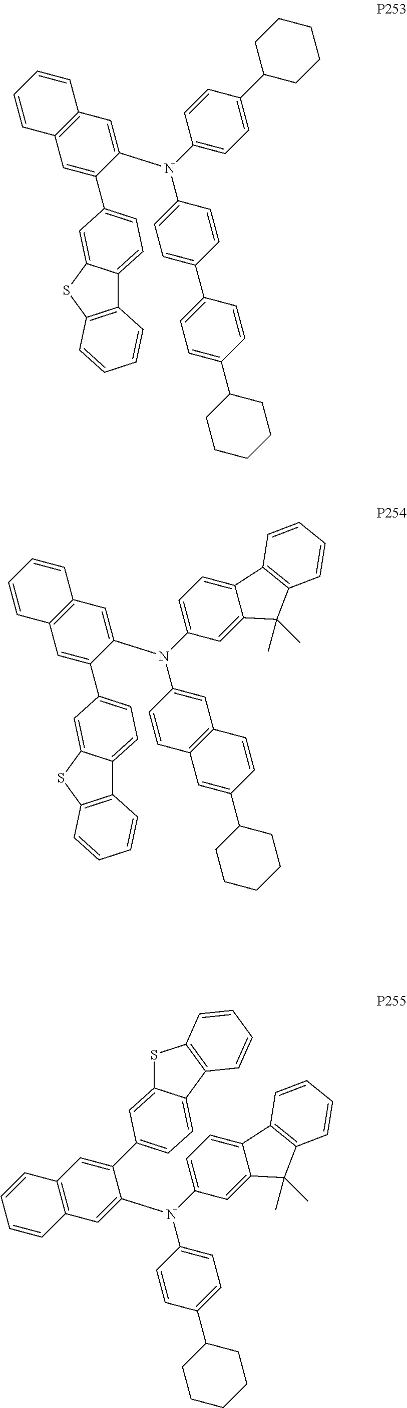 [SMILES: c1ccc2cc(N(c3ccc(-c4ccc(C5CCCCC5)cc4)cc3)c3ccc(C4CCCCC4)cc3)c(-c3ccc4c(c3)sc3ccccc34)cc2c1 ; CC1(C)c2ccccc2-c2ccc(N(c3ccc4cc(C5CCCCC5)ccc4c3)c3cc4ccccc4cc3-c3ccc4c(c3)sc3ccccc34)cc21 ; CC1(C)c2ccccc2-c2ccc(N(c3ccc(C4CCCCC4)cc3)c3cc4ccccc4cc3-c3ccc4c(c3)sc3ccccc34)cc21]

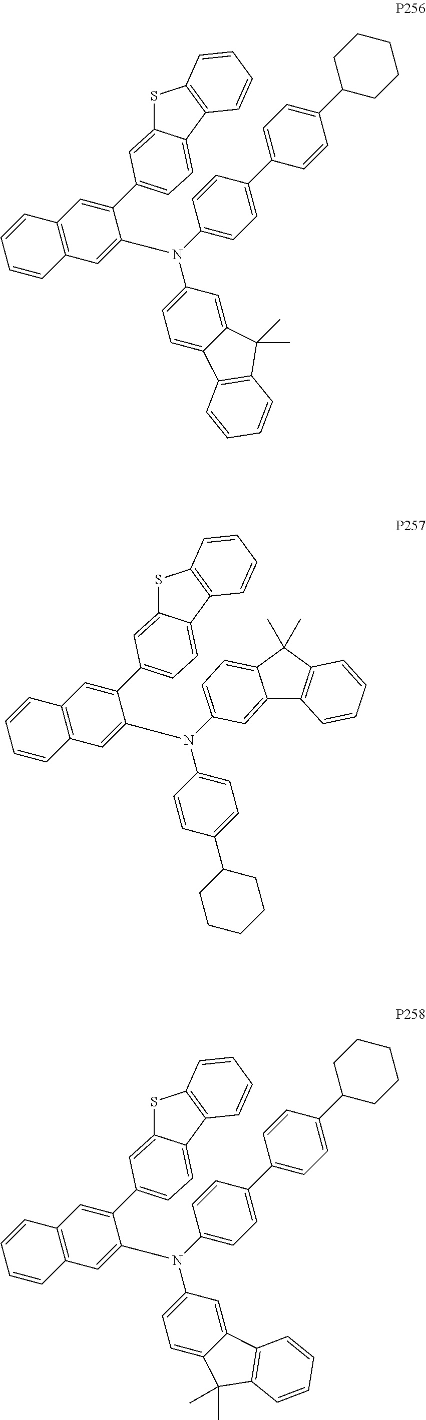 [SMILES: CC1(C)c2ccccc2-c2ccc(N(c3ccc(-c4ccc(C5CCCCC5)cc4)cc3)c3cc4ccccc4cc3-c3ccc4c(c3)sc3ccccc34)cc21 ; CC1(C)c2ccccc2-c2cc(N(c3ccc(C4CCCCC4)cc3)c3cc4ccccc4cc3-c3ccc4c(c3)sc3ccccc34)ccc21 ; CC1(C)c2ccccc2-c2cc(N(c3ccc(-c4ccc(C5CCCCC5)cc4)cc3)c3cc4ccccc4cc3-c3ccc4c(c3)sc3ccccc34)ccc21]

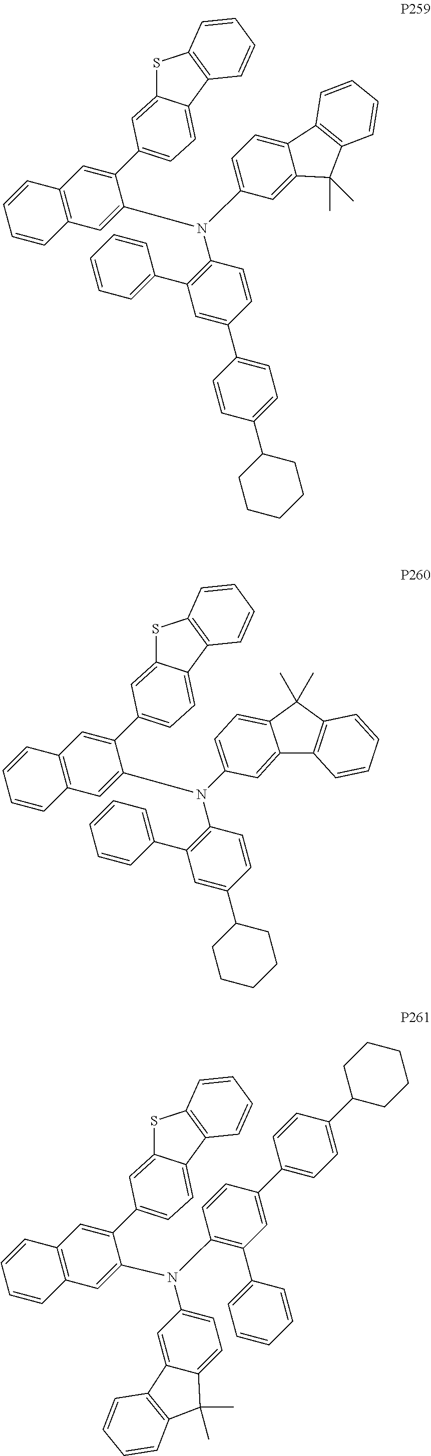 [SMILES: CC1(C)c2ccccc2-c2ccc(N(c3ccc(-c4ccc(C5CCCCC5)cc4)cc3-c3ccccc3)c3cc4ccccc4cc3-c3ccc4c(c3)sc3ccccc34)cc21 ; CC1(C)c2ccccc2-c2cc(N(c3ccc(C4CCCCC4)cc3-c3ccccc3)c3cc4ccccc4cc3-c3ccc4c(c3)sc3ccccc34)ccc21 ; CC1(C)c2ccccc2-c2cc(N(c3ccc(-c4ccc(C5CCCCC5)cc4)cc3-c3ccccc3)c3cc4ccccc4cc3-c3ccc4c(c3)sc3ccccc34)ccc21]

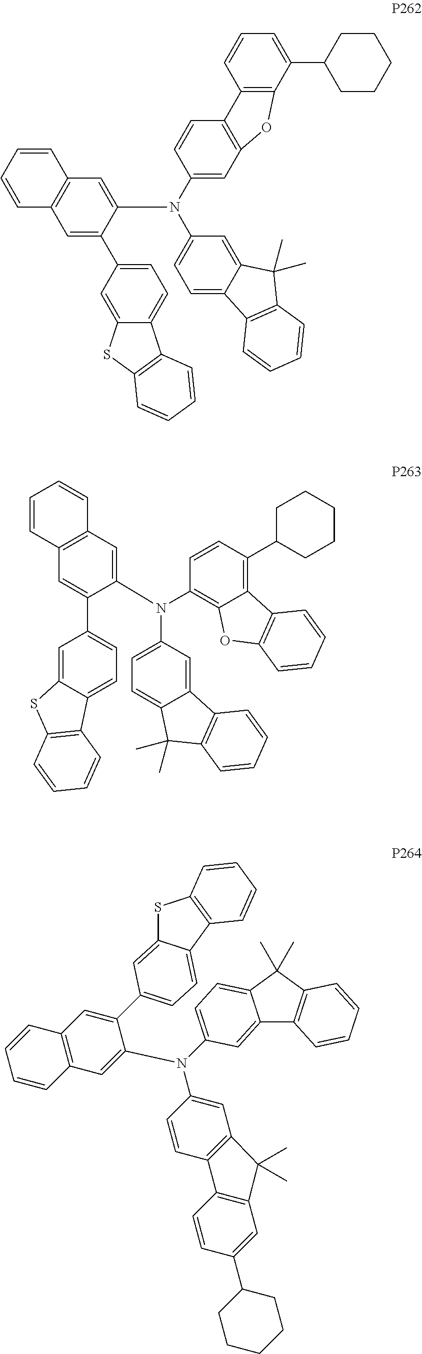 [SMILES: CC1(C)c2ccccc2-c2ccc(N(c3ccc4c(c3)oc3c(C5CCCCC5)cccc34)c3cc4ccccc4cc3-c3ccc4c(c3)sc3ccccc34)cc21 ; CC1(C)c2ccccc2-c2cc(N(c3cc4ccccc4cc3-c3ccc4c(c3)sc3ccccc34)c3ccc(C4CCCCC4)c4c3oc3ccccc34)ccc21 ; CC1(C)c2ccccc2-c2cc(N(c3ccc4c(c3)C(C)(C)c3cc(C5CCCCC5)ccc3-4)c3cc4ccccc4cc3-c3ccc4c(c3)sc3ccccc34)ccc21]

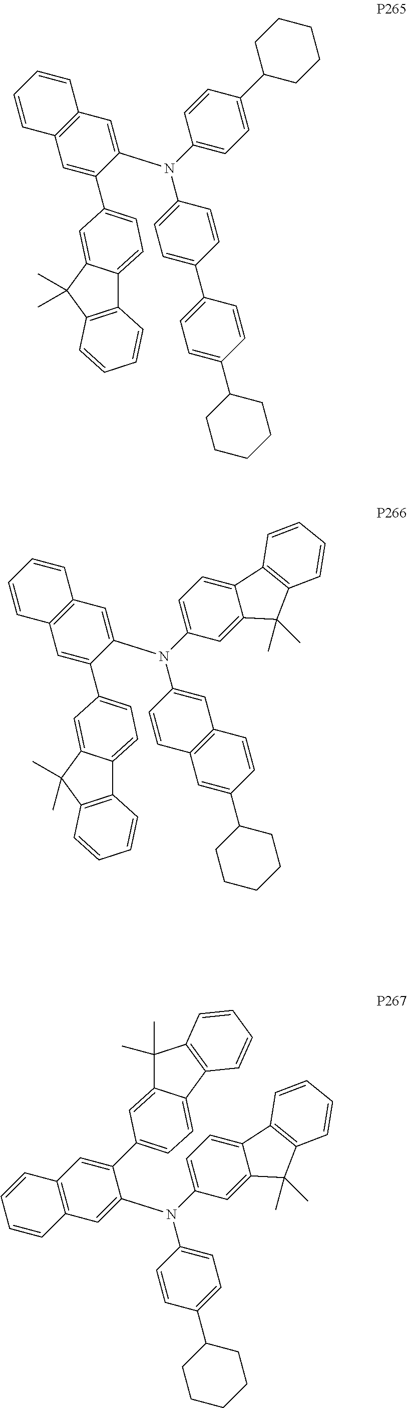 [SMILES: CC1(C)c2ccccc2-c2ccc(-c3cc4ccccc4cc3N(c3ccc(-c4ccc(C5CCCCC5)cc4)cc3)c3ccc(C4CCCCC4)cc3)cc21 ; CC1(C)c2ccccc2-c2ccc(-c3cc4ccccc4cc3N(c3ccc4c(c3)C(C)(C)c3ccccc3-4)c3ccc4cc(C5CCCCC5)ccc4c3)cc21 ; CC1(C)c2ccccc2-c2ccc(-c3cc4ccccc4cc3N(c3ccc(C4CCCCC4)cc3)c3ccc4c(c3)C(C)(C)c3ccccc3-4)cc21]

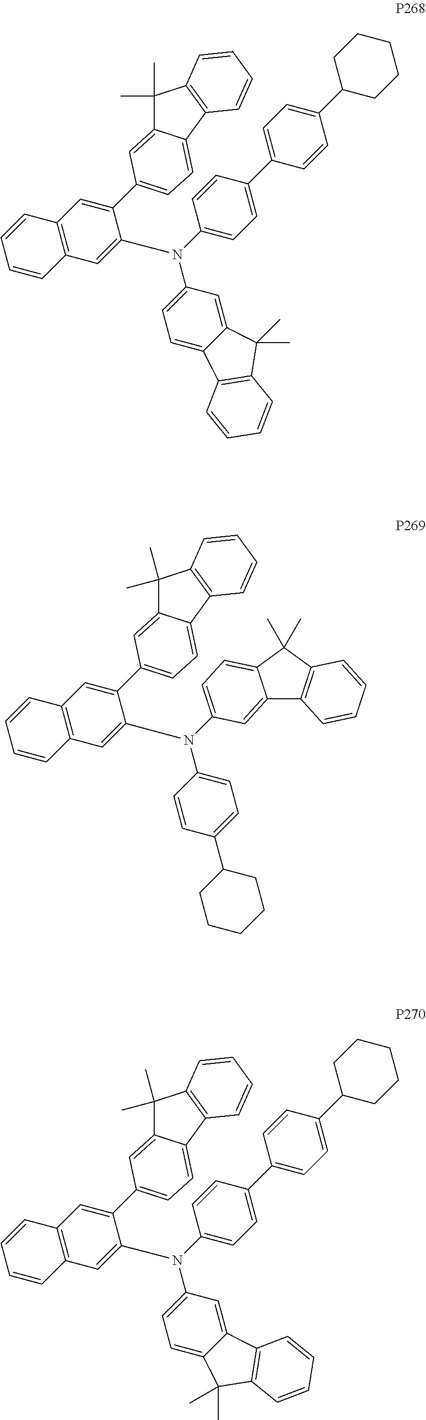 [SMILES: CC1(C)c2ccccc2-c2ccc(-c3cc4ccccc4cc3N(c3ccc(-c4ccc(C5CCCCC5)cc4)cc3)c3ccc4c(c3)C(C)(C)c3ccccc3-4)cc21 ; CC1(C)c2ccccc2-c2cc(N(c3ccc(C4CCCCC4)cc3)c3cc4ccccc4cc3-c3ccc4c(c3)C(C)(C)c3ccccc3-4)ccc21 ; CC1(C)c2ccccc2-c2cc(N(c3ccc(-c4ccc(C5CCCCC5)cc4)cc3)c3cc4ccccc4cc3-c3ccc4c(c3)C(C)(C)c3ccccc3-4)ccc21]

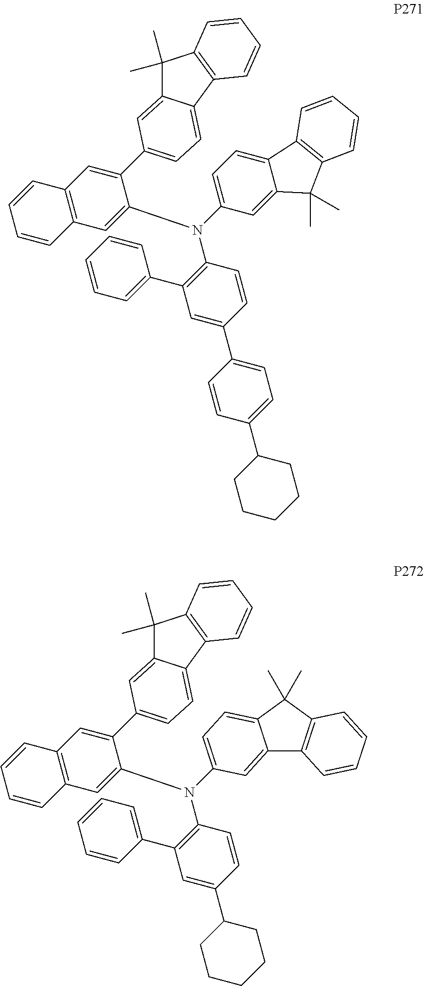 [SMILES: CC1(C)c2ccccc2-c2ccc(-c3cc4ccccc4cc3N(c3ccc4c(c3)C(C)(C)c3ccccc3-4)c3ccc(-c4ccc(C5CCCCC5)cc4)cc3-c3ccccc3)cc21 ; CC1(C)c2ccccc2-c2cc(N(c3ccc(C4CCCCC4)cc3-c3ccccc3)c3cc4ccccc4cc3-c3ccc4c(c3)C(C)(C)c3ccccc3-4)ccc21]

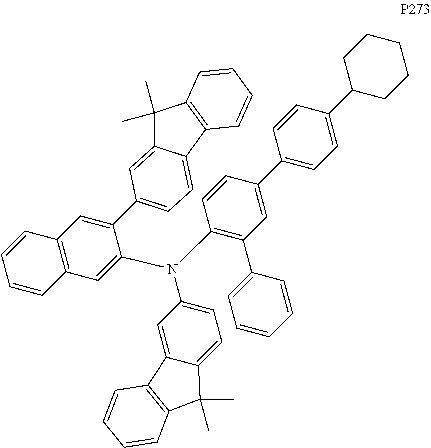 [SMILES: CC1(C)c2ccccc2-c2cc(N(c3ccc(-c4ccc(C5CCCCC5)cc4)cc3-c3ccccc3)c3cc4ccccc4cc3-c3ccc4c(c3)C(C)(C)c3ccccc3-4)ccc21]

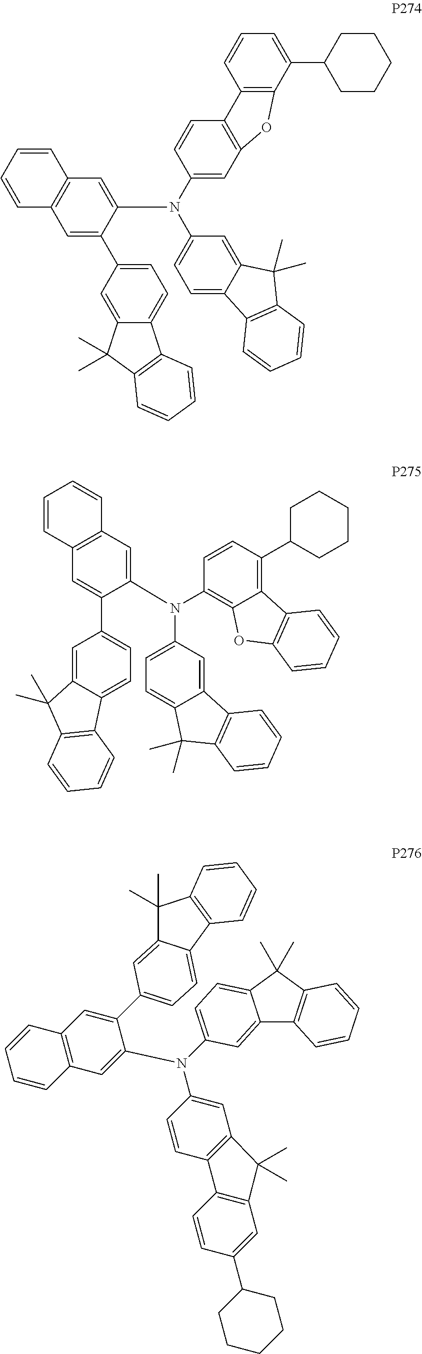 [SMILES: CC1(C)c2ccccc2-c2ccc(-c3cc4ccccc4cc3N(c3ccc4c(c3)C(C)(C)c3ccccc3-4)c3ccc4c(c3)oc3c(C5CCCCC5)cccc34)cc21 ; CC1(C)c2ccccc2-c2cc(N(c3cc4ccccc4cc3-c3ccc4c(c3)C(C)(C)c3ccccc3-4)c3ccc(C4CCCCC4)c4c3oc3ccccc34)ccc21 ; CC1(C)c2ccccc2-c2cc(N(c3ccc4c(c3)C(C)(C)c3cc(C5CCCCC5)ccc3-4)c3cc4ccccc4cc3-c3ccc4c(c3)C(C)(C)c3ccccc3-4)ccc21]

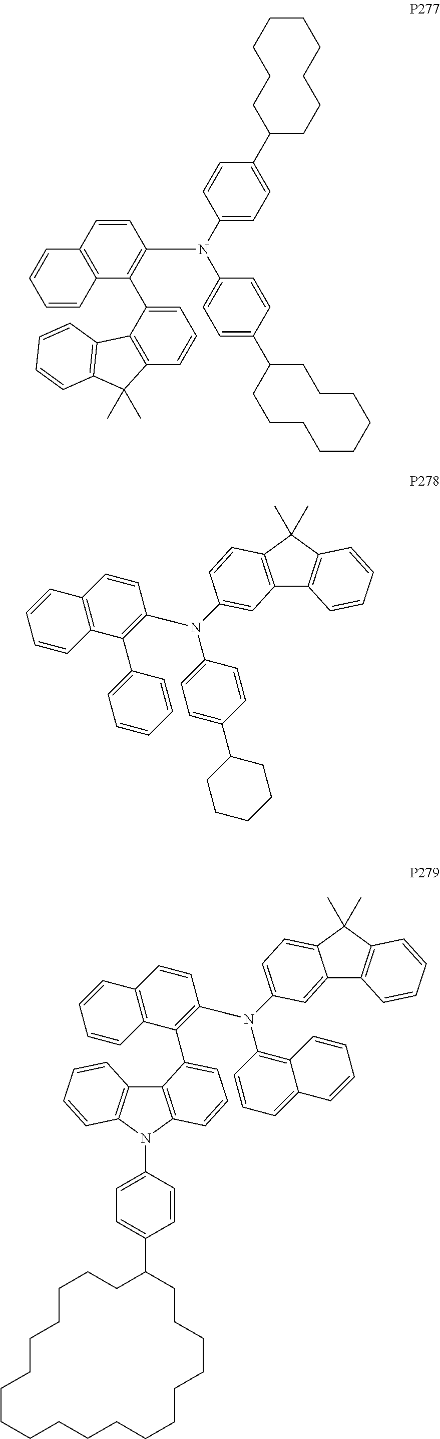 [SMILES: CC1(C)c2ccccc2-c2c(-c3c(N(c4ccc(C5CCCCCCCCC5)cc4)c4ccc(C5CCCCCCCCC5)cc4)ccc4ccccc34)cccc21 ; CC1(C)c2ccccc2-c2cc(N(c3ccc(C4CCCCC4)cc3)c3ccc4ccccc4c3-c3ccccc3)ccc21 ; CC1(C)c2ccccc2-c2cc(N(c3ccc4ccccc4c3-c3cccc4c3c3ccccc3n4-c3ccc(C4CCCCCCCCCCCCCCCCCCC4)cc3)c3cccc4ccccc34)ccc21]

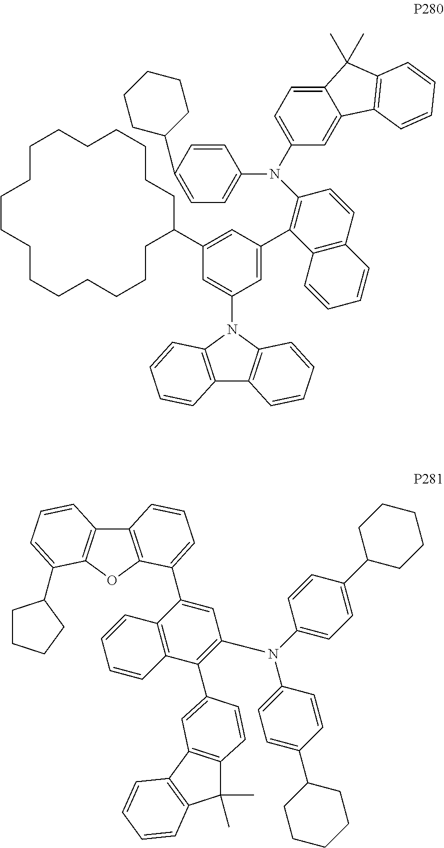 [SMILES: CC1(C)c2ccccc2-c2cc(N(c3ccc(C4CCCCC4)cc3)c3ccc4ccccc4c3-c3cc(C4CCCCCCCCCCCCCCCCC4)cc(-n4c5ccccc5c5ccccc54)c3)ccc21 ; CC1(C)c2ccccc2-c2cc(-c3c(N(c4ccc(C5CCCCC5)cc4)c4ccc(C5CCCCC5)cc4)cc(-c4cccc5c4oc4c(C6CCCC6)cccc45)c4ccccc34)ccc21]

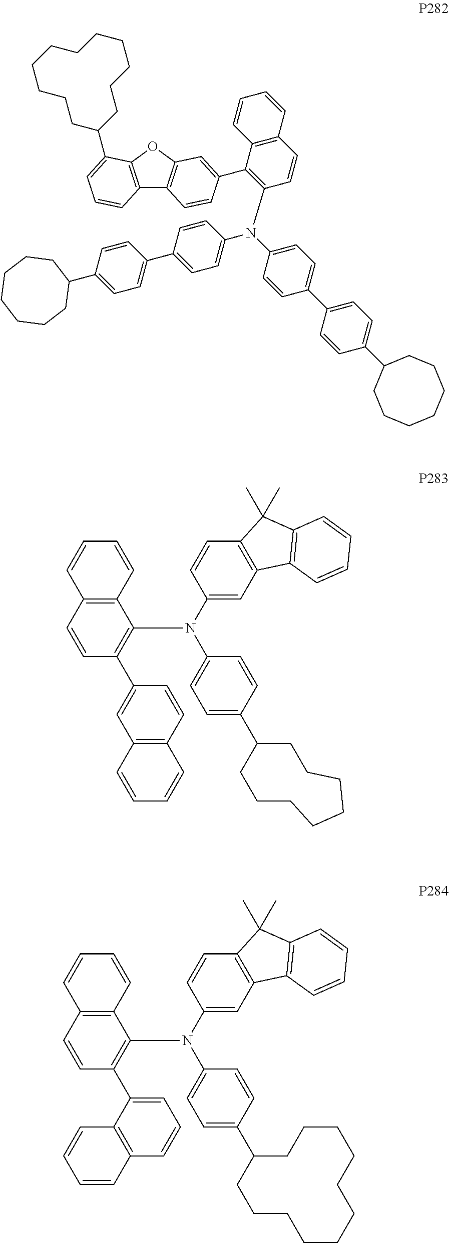 [SMILES: c1ccc2c(-c3ccc4c(c3)oc3c(C5CCCCCCCCCCC5)cccc34)c(N(c3ccc(-c4ccc(C5CCCCCCC5)cc4)cc3)c3ccc(-c4ccc(C5CCCCCCC5)cc4)cc3)ccc2c1 ; CC1(C)c2ccccc2-c2cc(N(c3ccc(C4CCCCCCCC4)cc3)c3c(-c4ccc5ccccc5c4)ccc4ccccc34)ccc21 ; CC1(C)c2ccccc2-c2cc(N(c3ccc(C4CCCCCCCCCCC4)cc3)c3c(-c4cccc5ccccc45)ccc4ccccc34)ccc21]

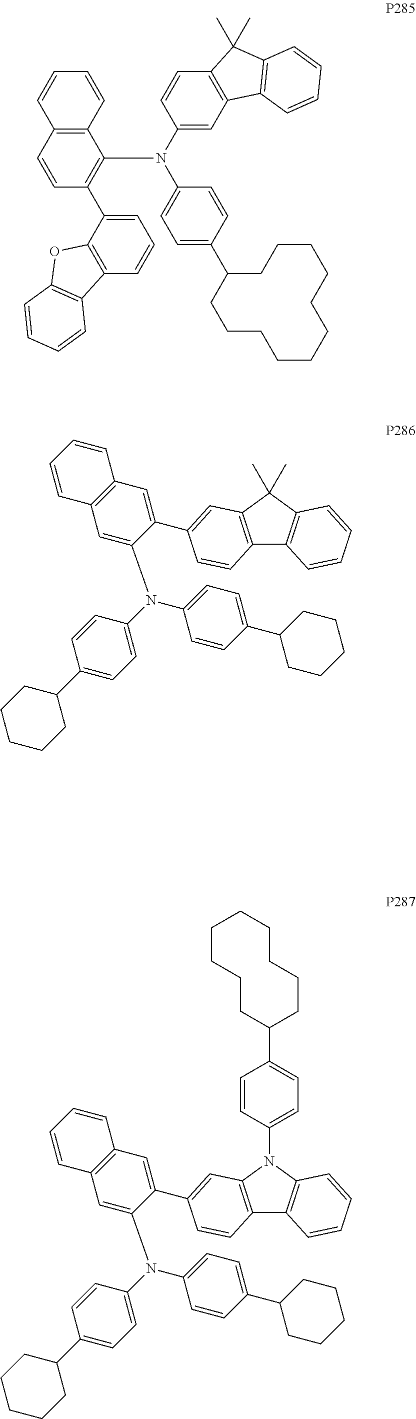 [SMILES: CC1(C)c2ccccc2-c2cc(N(c3ccc(C4CCCCCCCCCCC4)cc3)c3c(-c4cccc5c4oc4ccccc45)ccc4ccccc34)ccc21 ; CC1(C)c2ccccc2-c2ccc(-c3cc4ccccc4cc3N(c3ccc(C4CCCCC4)cc3)c3ccc(C4CCCCC4)cc3)cc21 ; c1ccc2cc(N(c3ccc(C4CCCCC4)cc3)c3ccc(C4CCCCC4)cc3)c(-c3ccc4c5ccccc5n(-c5ccc(C6CCCCCCCCC6)cc5)c4c3)cc2c1]

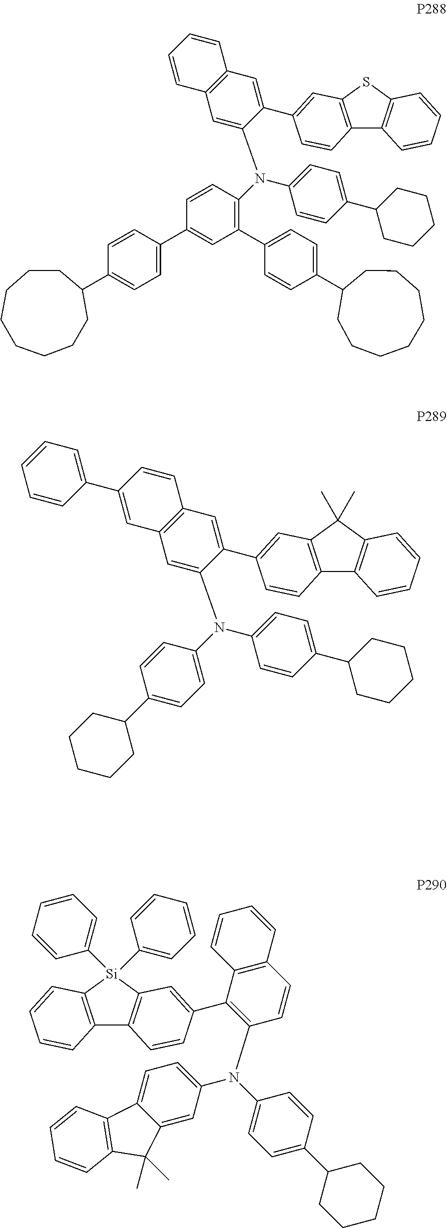 [SMILES: c1ccc2cc(N(c3ccc(C4CCCCC4)cc3)c3ccc(-c4ccc(C5CCCCCCCC5)cc4)cc3-c3ccc(C4CCCCCCCC4)cc3)c(-c3ccc4c(c3)sc3ccccc34)cc2c1 ; CC1(C)c2ccccc2-c2ccc(-c3cc4ccc(-c5ccccc5)cc4cc3N(c3ccc(C4CCCCC4)cc3)c3ccc(C4CCCCC4)cc3)cc21 ; CC1(C)c2ccccc2-c2ccc(N(c3ccc(C4CCCCC4)cc3)c3ccc4ccccc4c3-c3ccc4c(c3)[Si](c3ccccc3)(c3ccccc3)c3ccccc3-4)cc21]

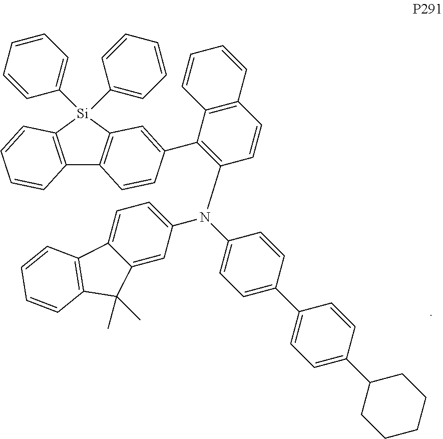 [SMILES: CC1(C)c2ccccc2-c2ccc(N(c3ccc(-c4ccc(C5CCCCC5)cc4)cc3)c3ccc4ccccc4c3-c3ccc4c(c3)[Si](c3ccccc3)(c3ccccc3)c3ccccc3-4)cc21]

5. The compound according to claim 1, wherein the compound has a structure as shown in Formula (II): 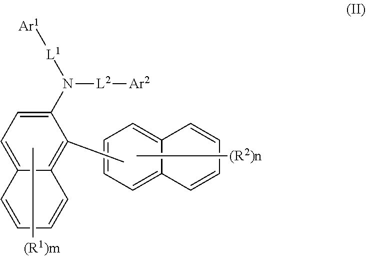
wherein, L¹ and L² are each independently selected from a single bond, a substituted or unsubstituted C₆-C₅₀ alkylene, a substituted or unsubstituted C₃-C₃₀ heteroarylene group;
Ar¹ and Ar² are each independently selected from H, a substituted or unsubstituted C₆-C₅₀ aryl, a substituted or unsubstituted C₆-C₅₀ fused aryl, a substituted or unsubstituted C₃-C₃₀ heteroaryl, a substituted or unsubstituted C₃-C₃₀ fused heteroaryl; and when Ar¹ is H, L¹ is not a single bond, and when Ar² is H, L² is not a single bond;
R¹ and R² are each independently selected from H, a halogen, carbonyl, carboxyl, cyano, amido, a C₁-C₂₀ alkyl, a C₃-C₂₀ cycloalkyl, a C₂-C₁₂ alkenyl, a C₂-C₁₂ alkynyl, a C₁-C₁₂ alkoxy, a substituted or unsubstituted C₆-C₅₀ aryl, a substituted or unsubstituted C₃-C₃₀ heteroaryl, a C₆-C₅₀ fused aryl; and R¹ and R² are linked on the naphthalene ring in a single bond way;
m is an integer of 0-6, and n is an integer of 0-7;
when the above-mentioned groups have a substituent, the substituent is each independently selected from one or more of a halogen, carbonyl, carboxyl, cyano, amido, a C₁-C₁₀ alkyl, a C₃-C₁₀ cycloalkyl, a C₂-C₁₀ alkenyl, a C₁-C₆ alkoxy, a C₁-C₆ thioalkoxy, a C₆-C₃₀ monocyclic or fused-cyclic aryl, a C₃-C₃₀ monocyclic or fused-cyclic heteroaryl.

6. The compound according to claim 5, wherein
L¹ and L² are a single bond; R¹ and R² are H; Ar¹ and Ar² are each independently selected from a C₆-C₅₀ aryl or fused aryl, a C₃-C₃₀ heteroaryl or fused heteroaryl;
preferably, Ar¹ and Ar² are each independently selected from the group consisting of substituted or unsubstituted: 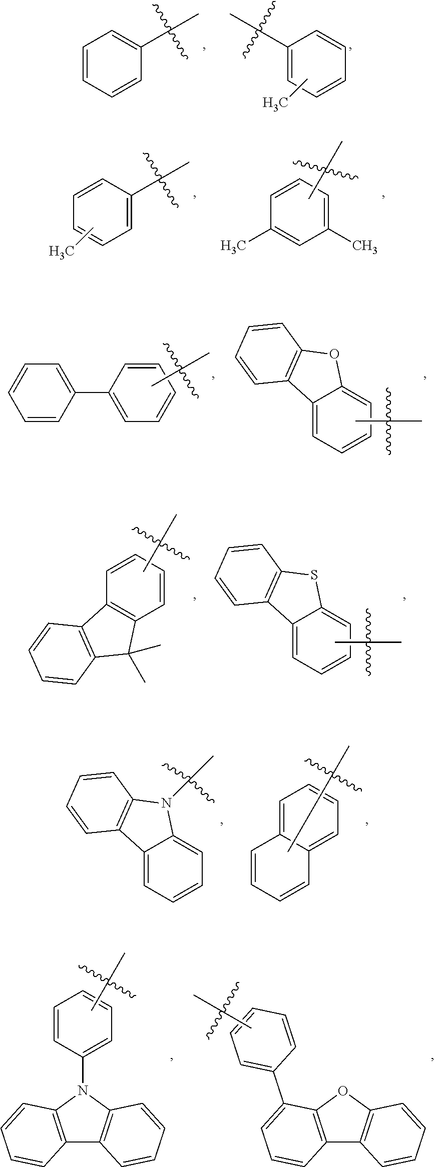 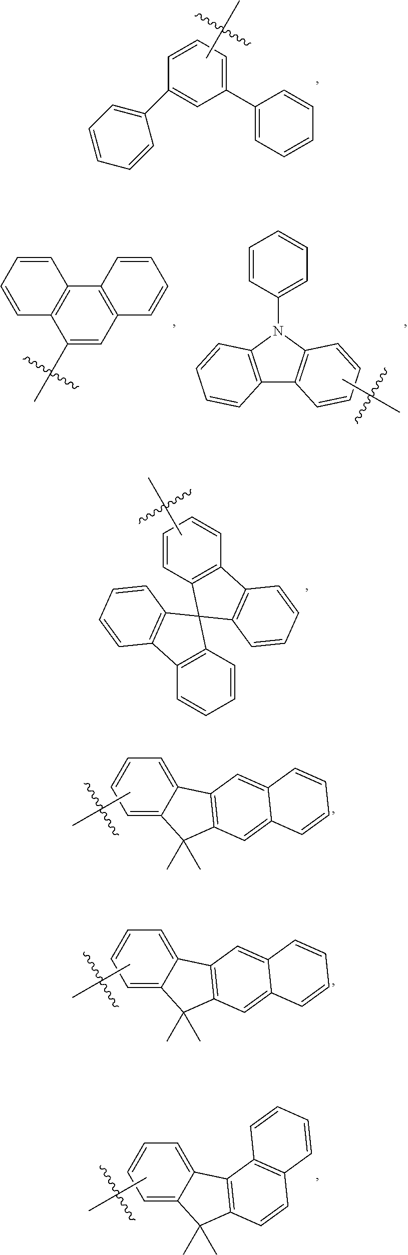 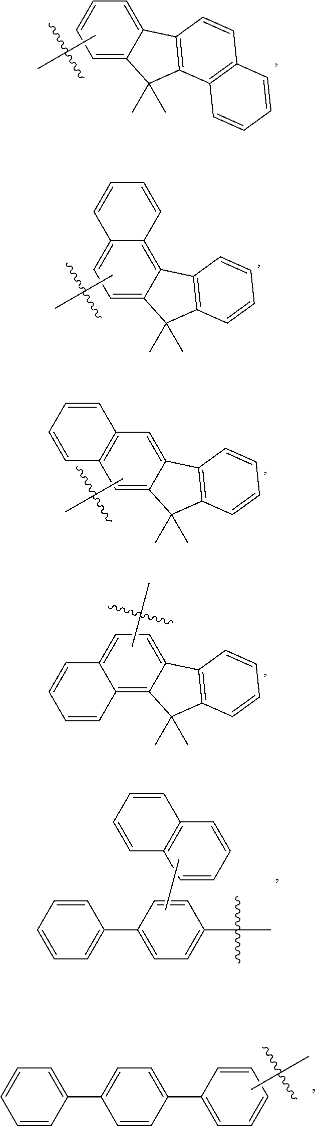 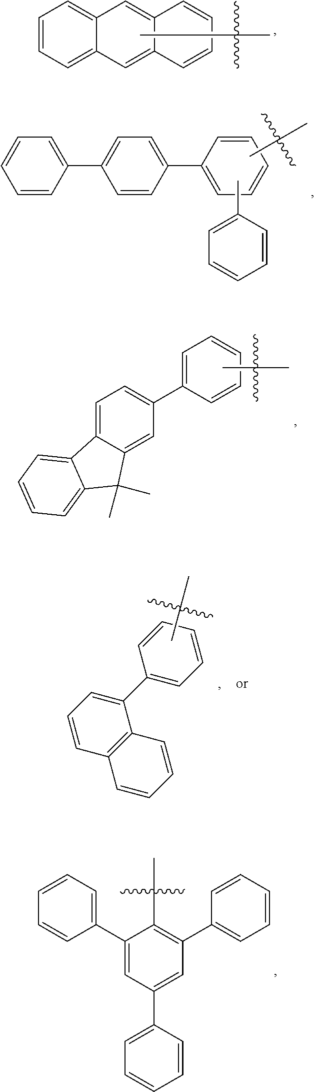
wherein, [symbol] represents an access position of a group;
more preferably, Ar¹ and Ar² are each independently selected from the group consisting of substituted or unsubstituted:

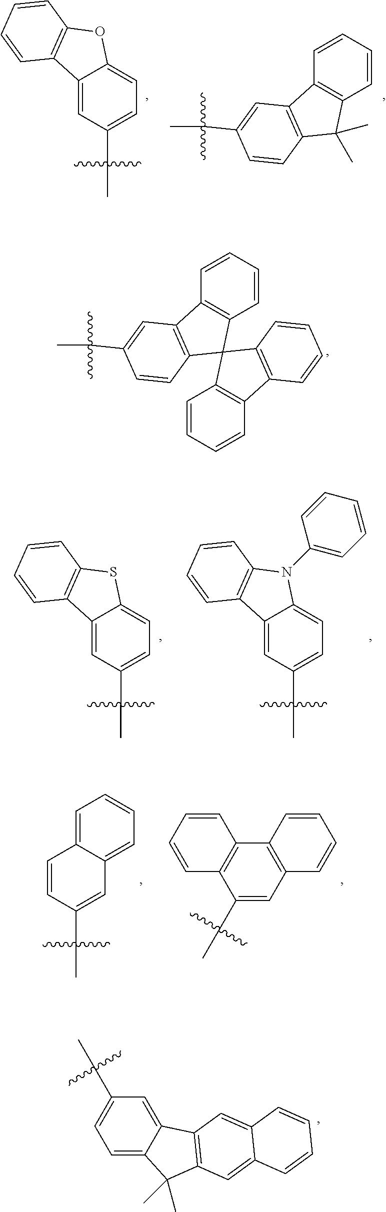

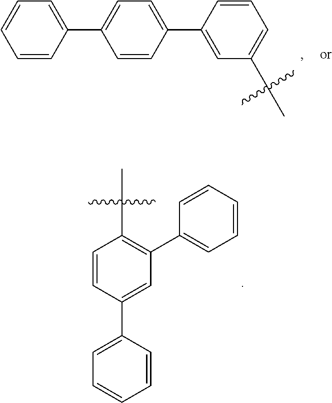 [SMILES: *c1cccc(-c2ccc(-c3ccccc3)cc2)c1 ; *c1ccc(-c2ccccc2)cc1-c1ccccc1]

7. The compound according to claim 4, wherein the compound has a structure as shown in N1-N419:

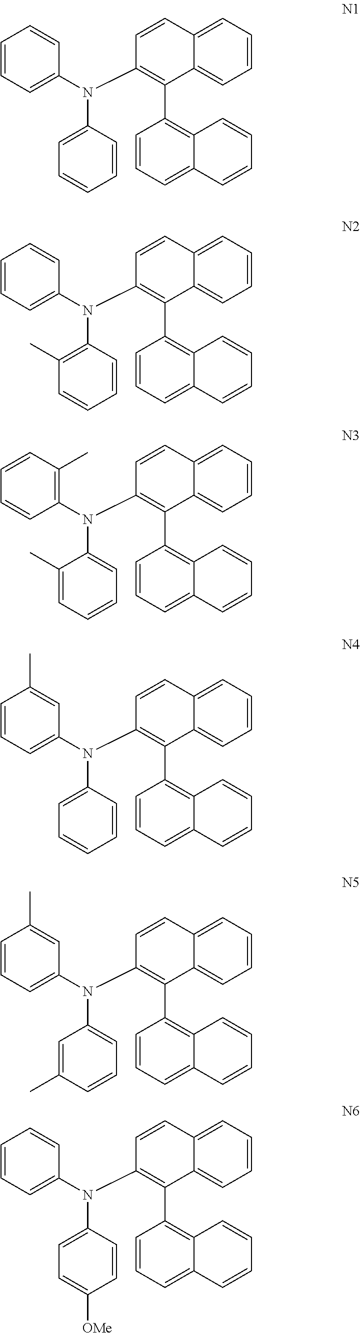

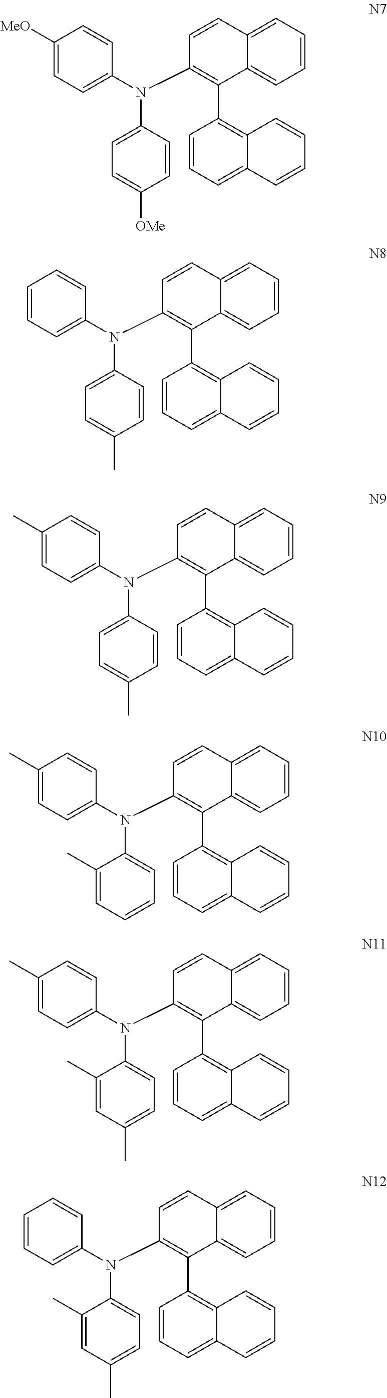

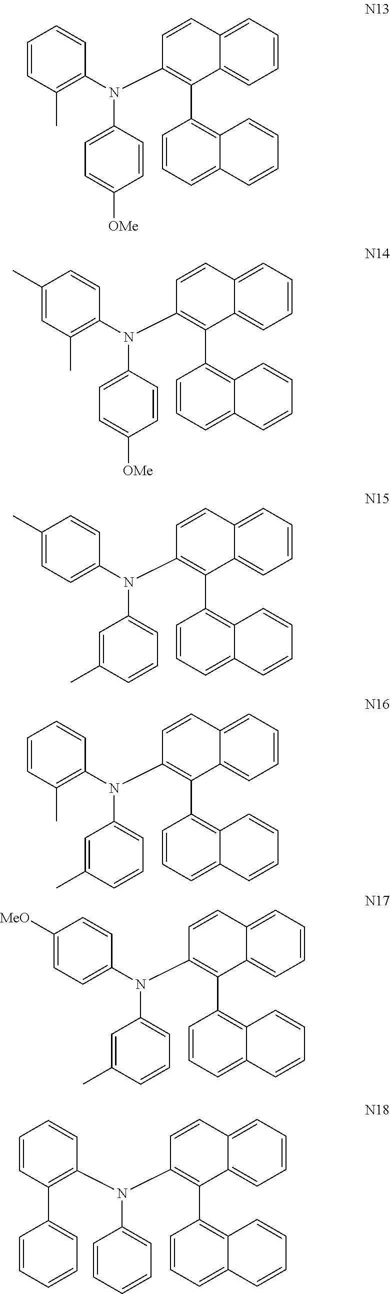

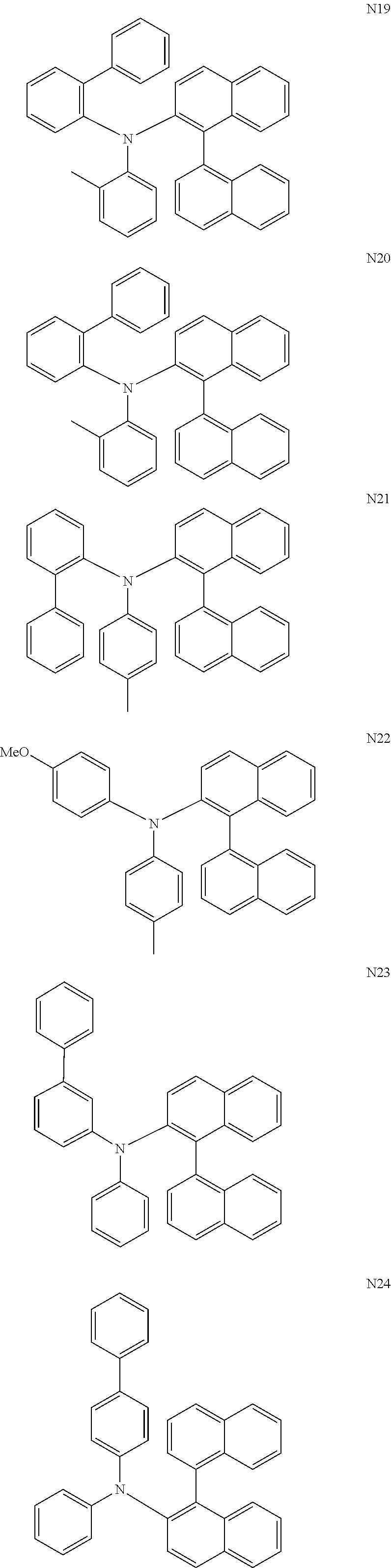

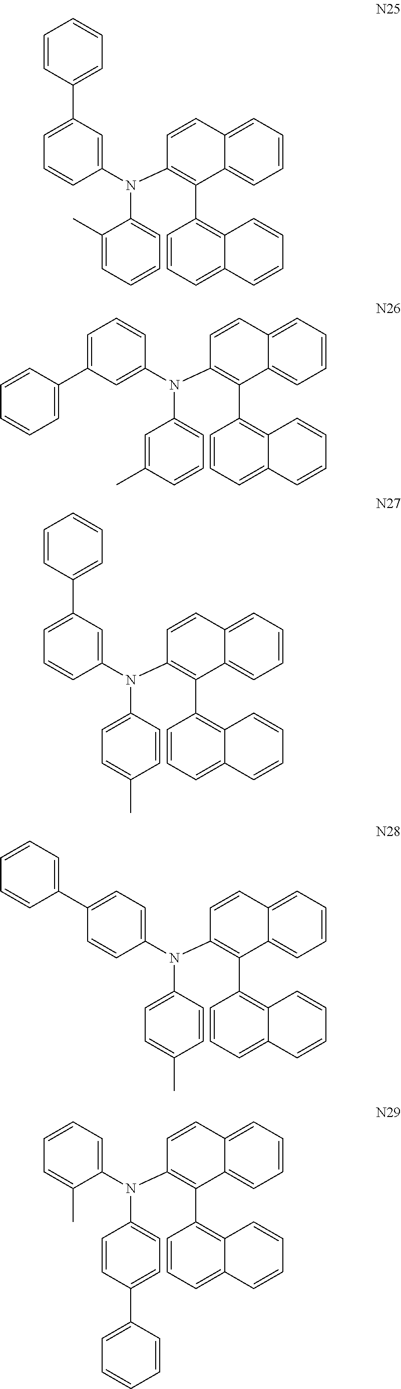

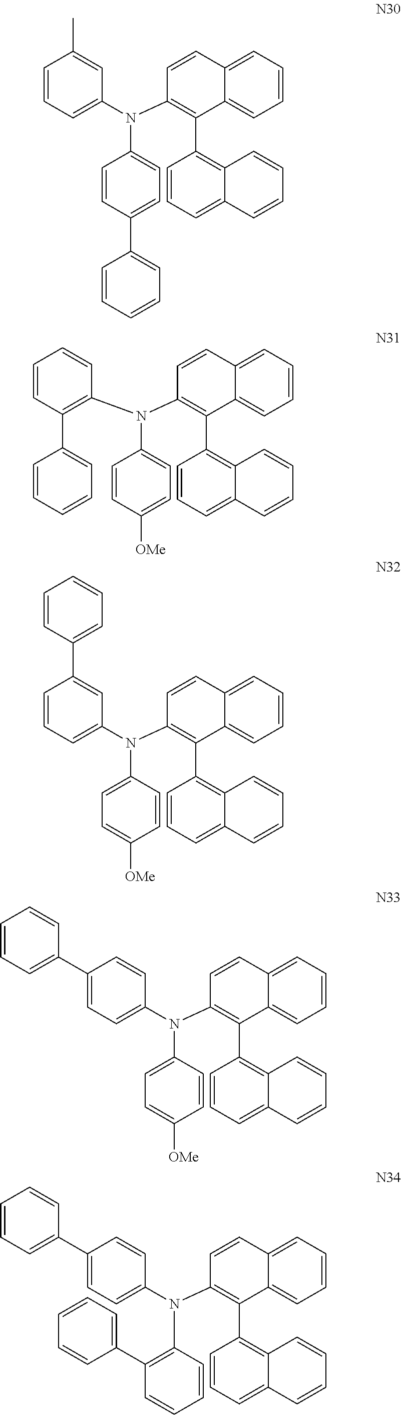 [SMILES: Cc1cccc(N(c2ccc(-c3ccccc3)cc2)c2ccc3ccccc3c2-c2cccc3ccccc23)c1 ; COc1ccc(N(c2ccccc2-c2ccccc2)c2ccc3ccccc3c2-c2cccc3ccccc23)cc1 ; COc1ccc(N(c2cccc(-c3ccccc3)c2)c2ccc3ccccc3c2-c2cccc3ccccc23)cc1 ; COc1ccc(N(c2ccc(-c3ccccc3)cc2)c2ccc3ccccc3c2-c2cccc3ccccc23)cc1 ; c1ccc(-c2ccc(N(c3ccccc3-c3ccccc3)c3ccc4ccccc4c3-c3cccc4ccccc34)cc2)cc1]

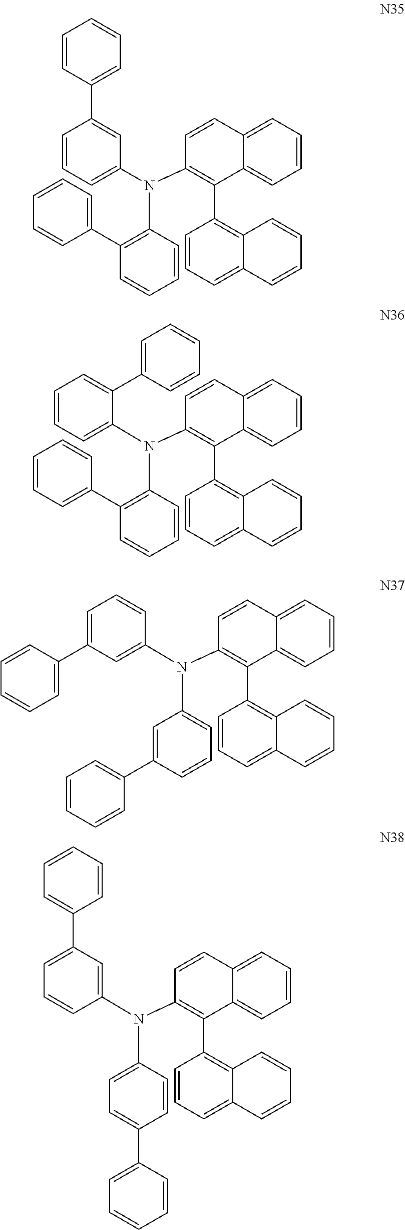

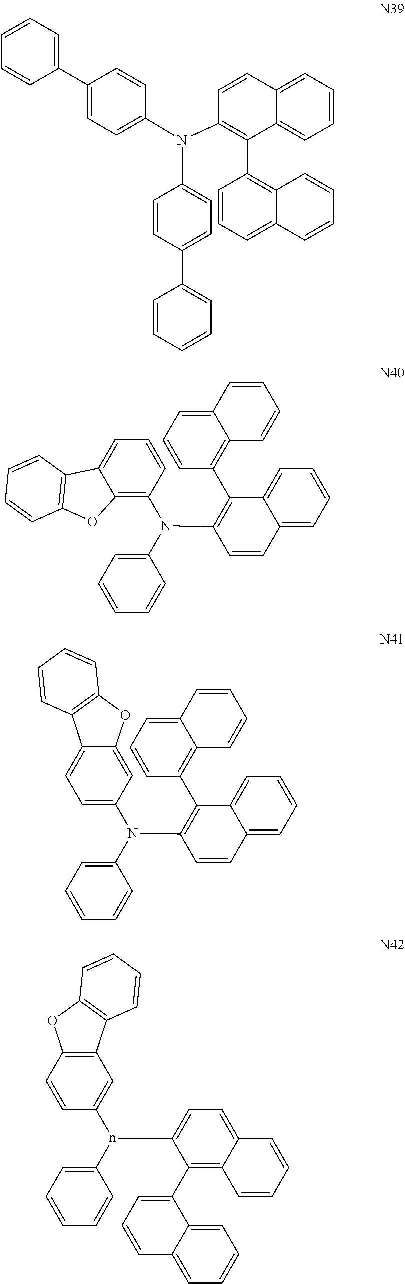

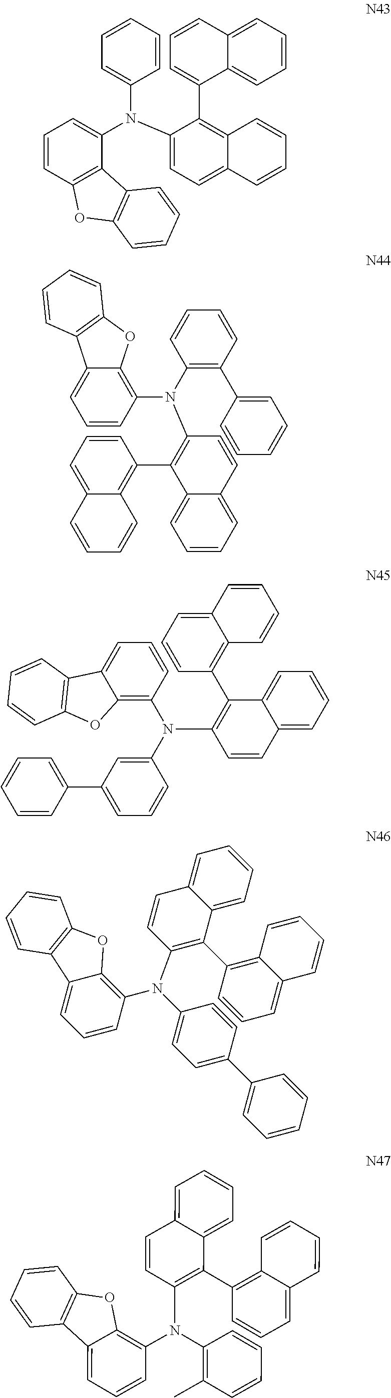

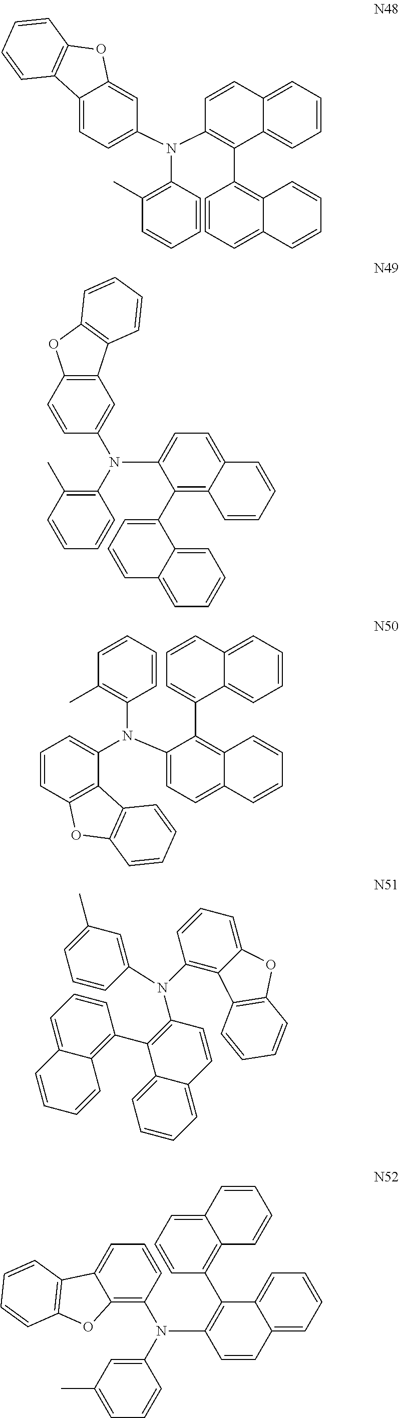

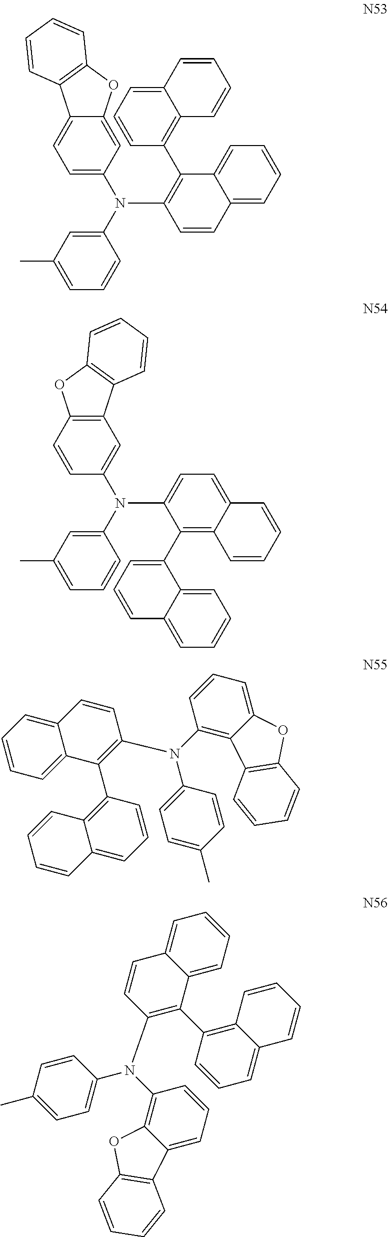

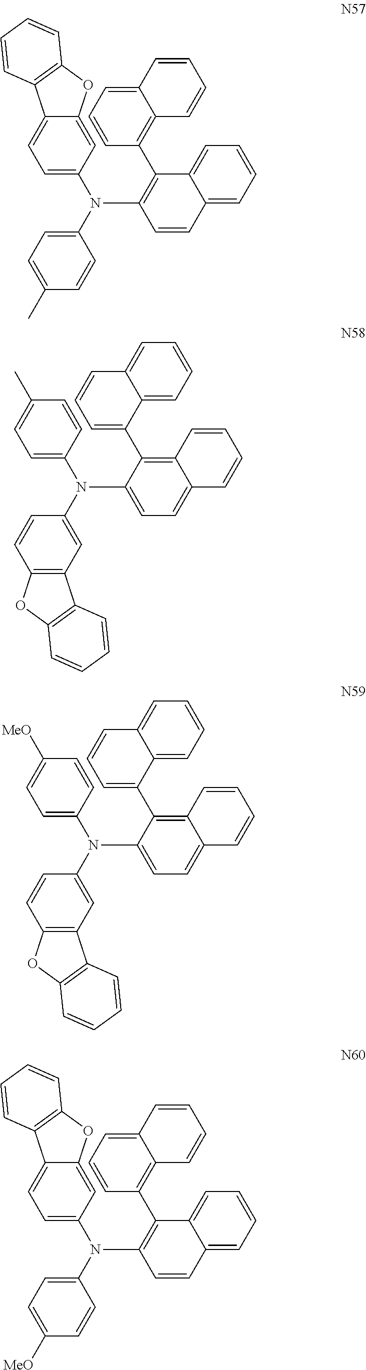 [SMILES: Cc1ccc(N(c2ccc3c(c2)oc2ccccc23)c2ccc3ccccc3c2-c2cccc3ccccc23)cc1 ; Cc1ccc(N(c2ccc3oc4ccccc4c3c2)c2ccc3ccccc3c2-c2cccc3ccccc23)cc1 ; COc1ccc(N(c2ccc3oc4ccccc4c3c2)c2ccc3ccccc3c2-c2cccc3ccccc23)cc1 ; COc1ccc(N(c2ccc3c(c2)oc2ccccc23)c2ccc3ccccc3c2-c2cccc3ccccc23)cc1]

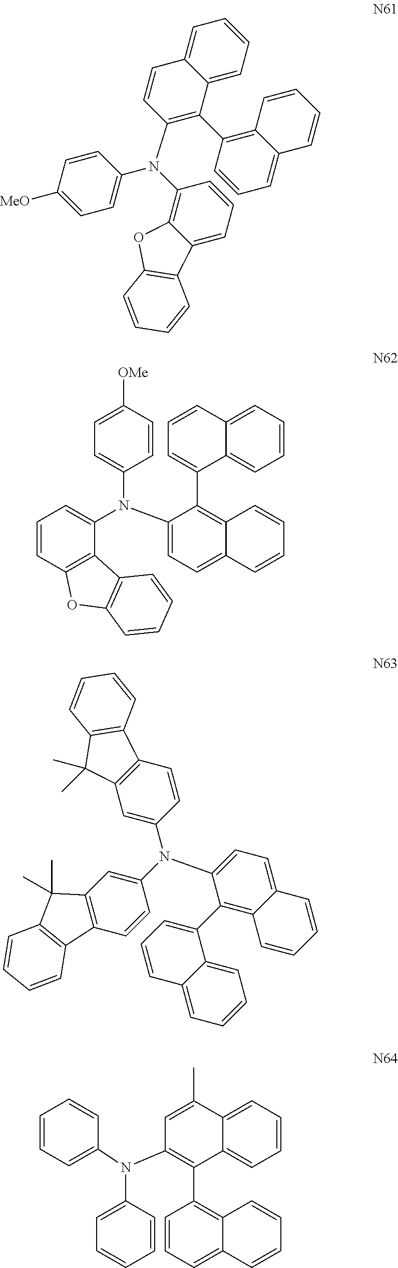

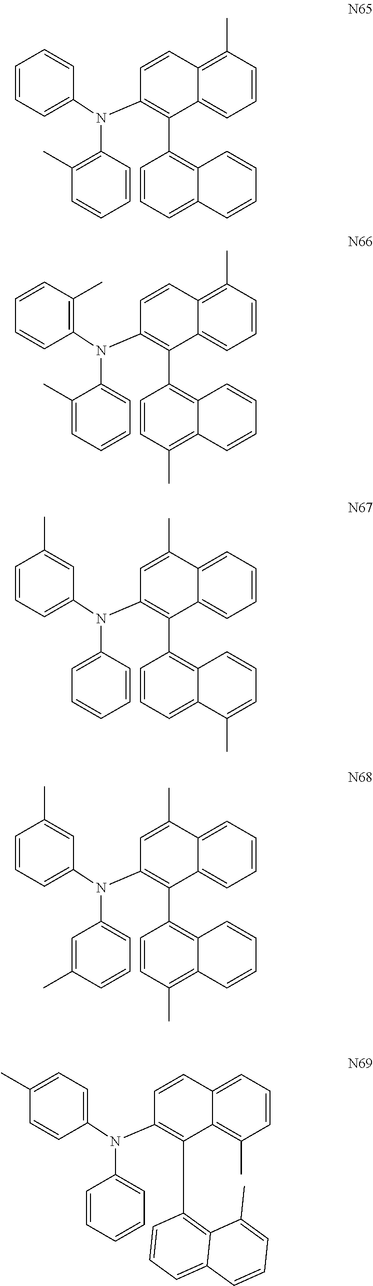

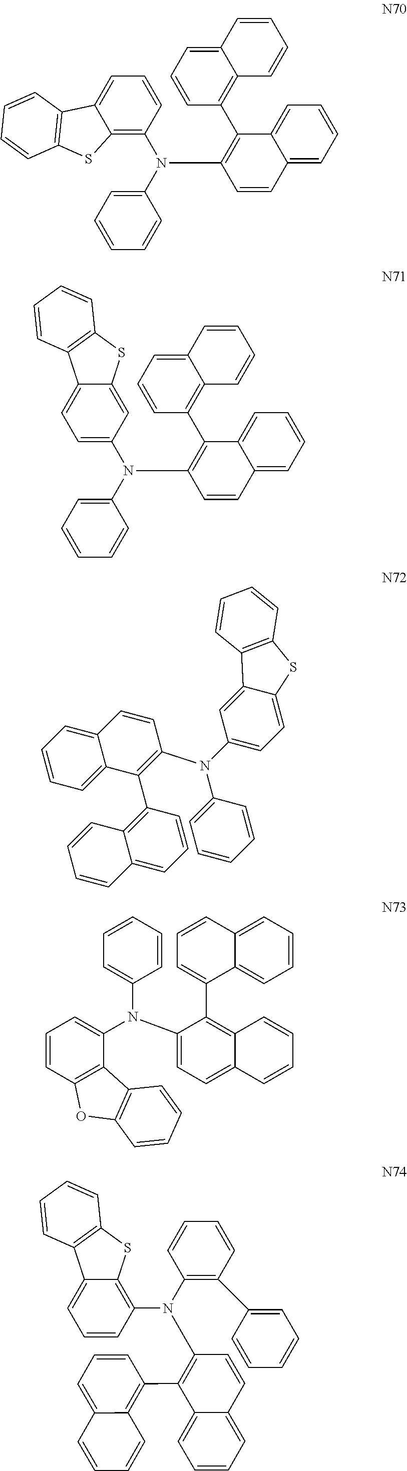

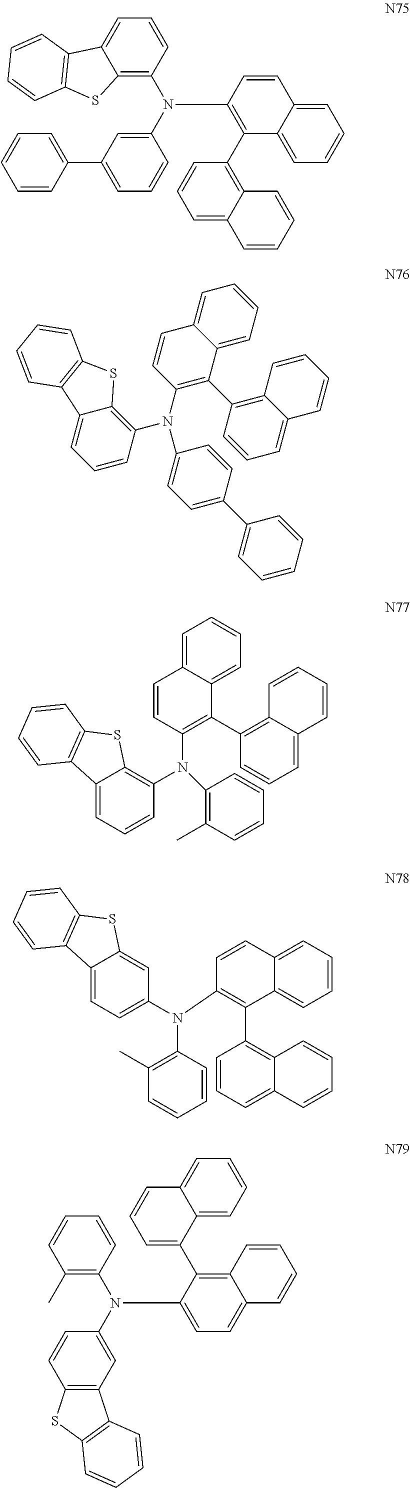

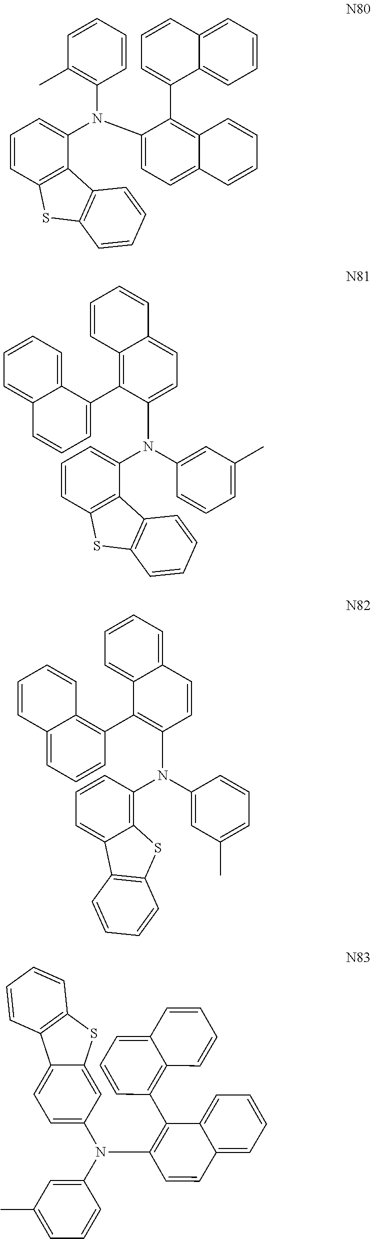

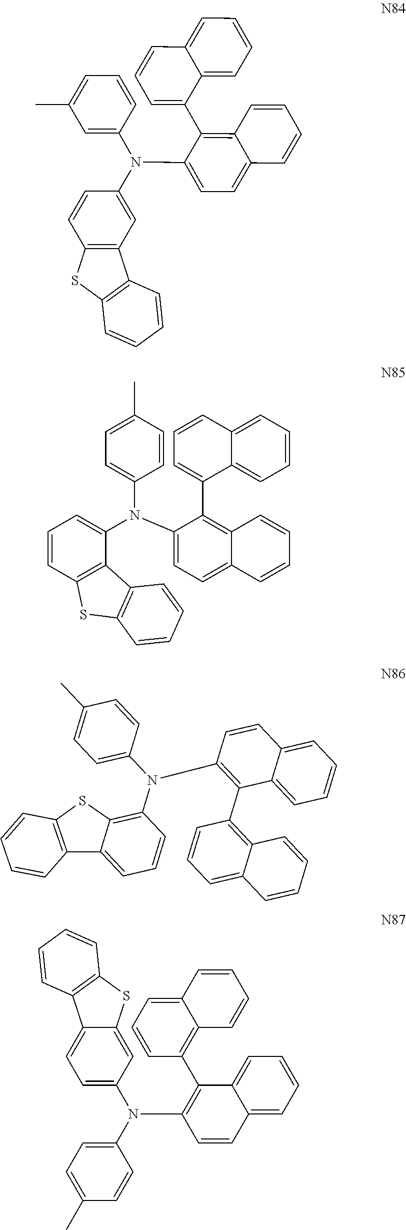

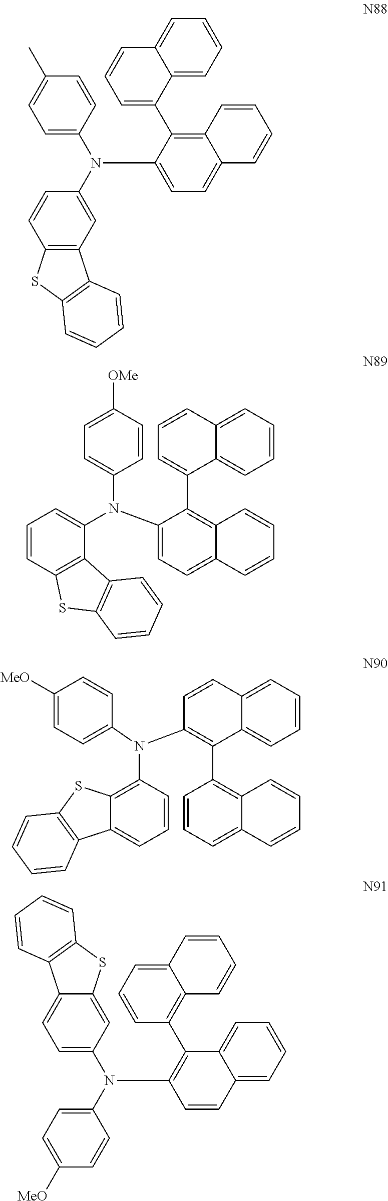

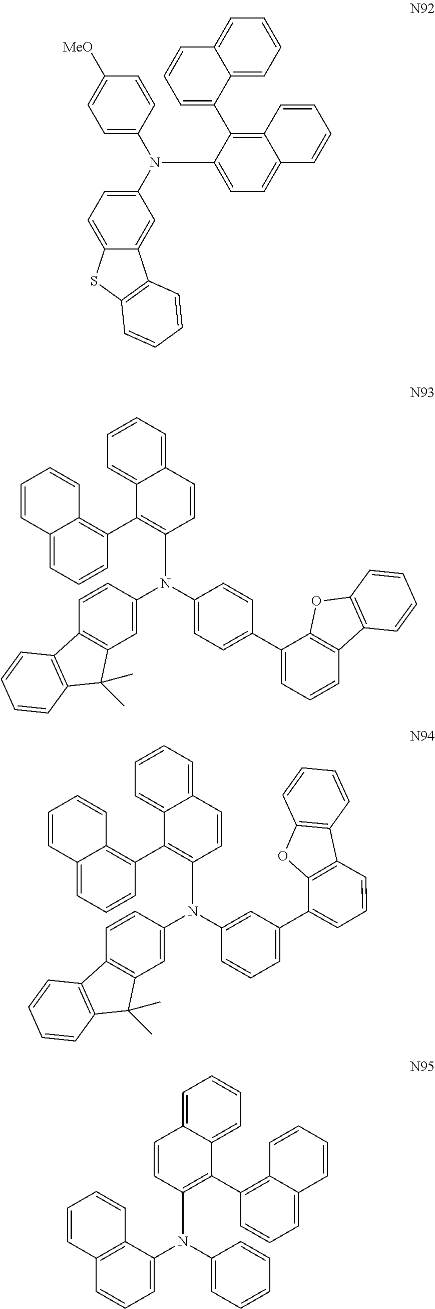 [SMILES: COc1ccc(N(c2ccc3sc4ccccc4c3c2)c2ccc3ccccc3c2-c2cccc3ccccc23)cc1 ; CC1(C)c2ccccc2-c2ccc(N(c3ccc(-c4cccc5c4oc4ccccc45)cc3)c3ccc4ccccc4c3-c3cccc4ccccc34)cc21 ; CC1(C)c2ccccc2-c2ccc(N(c3cccc(-c4cccc5c4oc4ccccc45)c3)c3ccc4ccccc4c3-c3cccc4ccccc34)cc21 ; c1ccc(N(c2ccc3ccccc3c2-c2cccc3ccccc23)c2cccc3ccccc23)cc1]

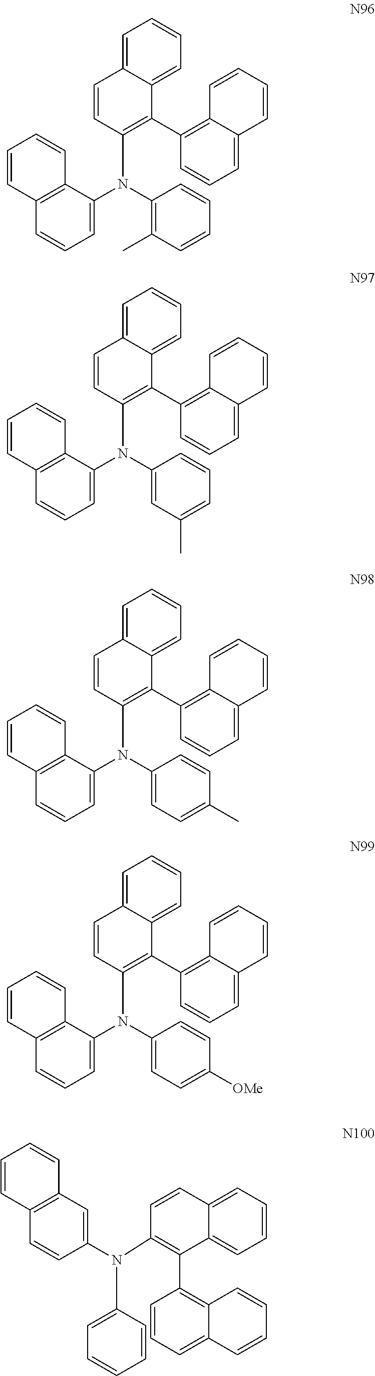

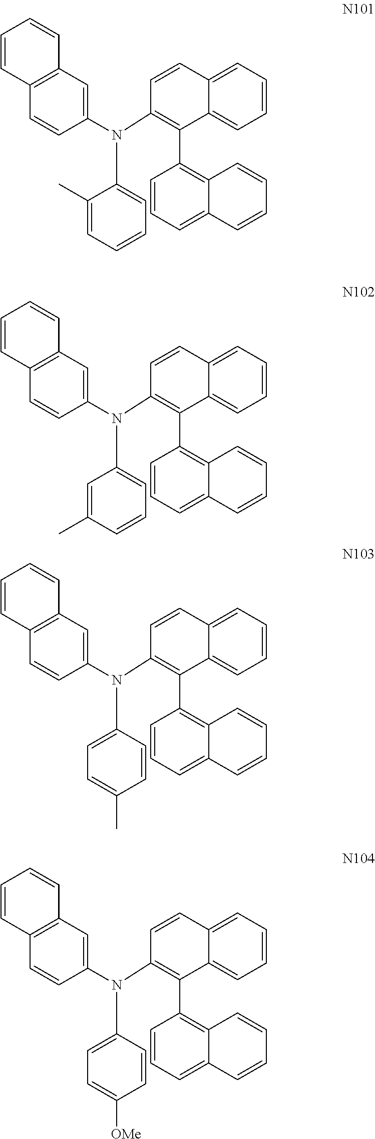

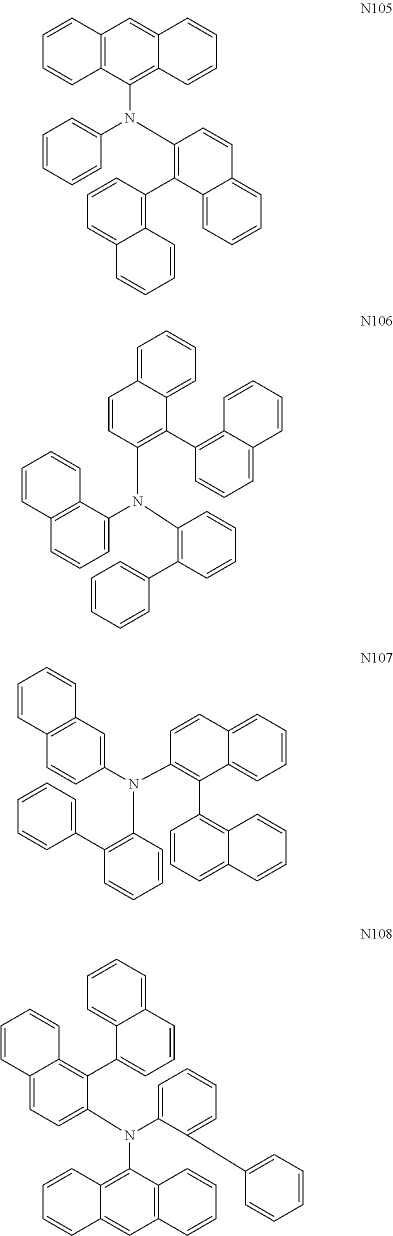

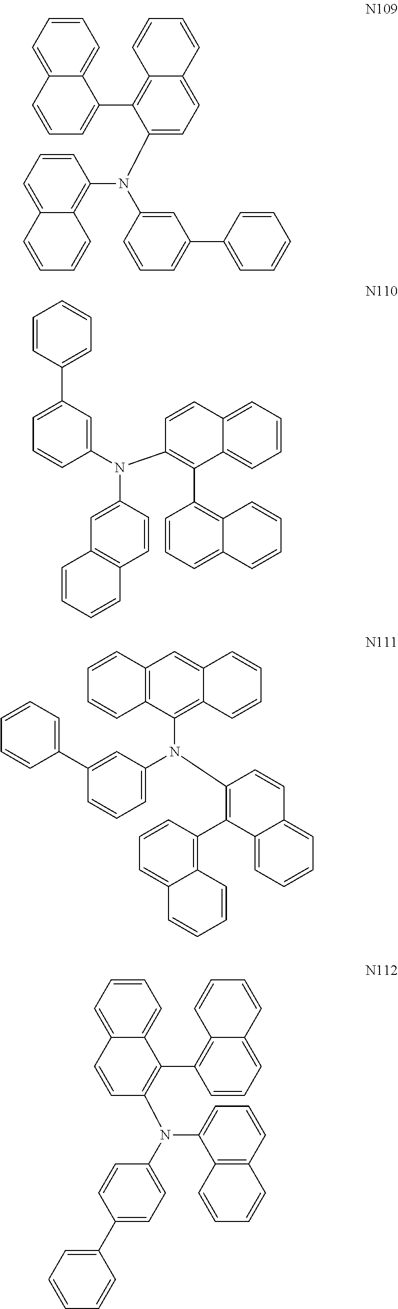

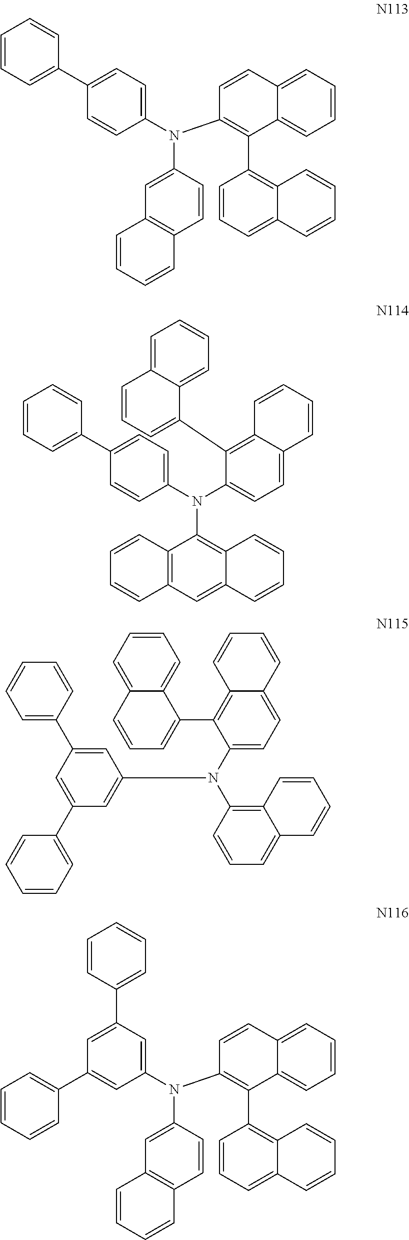

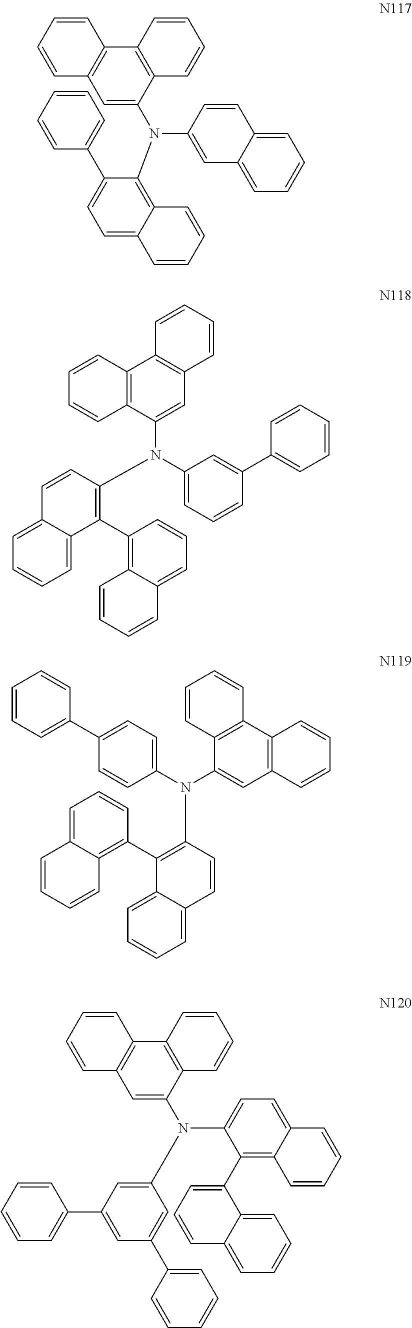

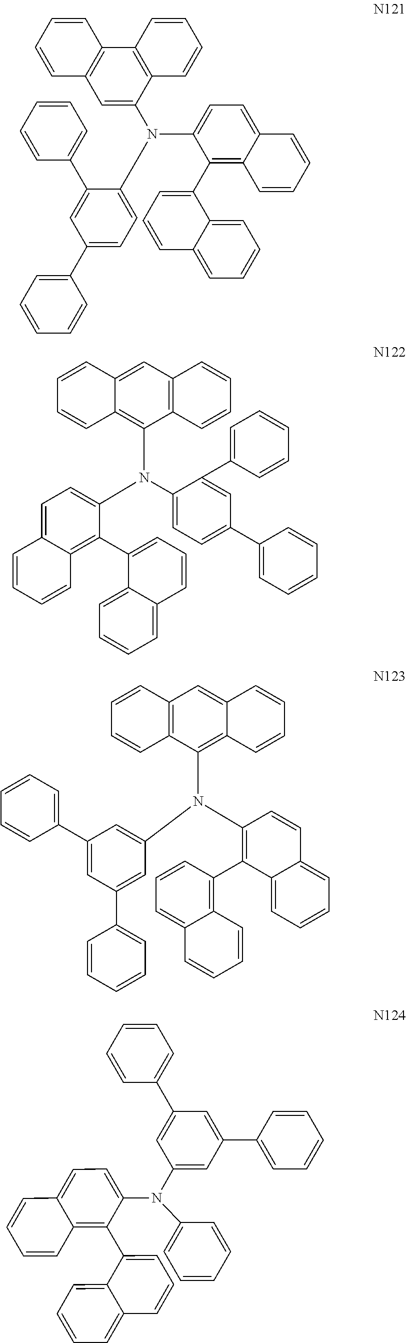

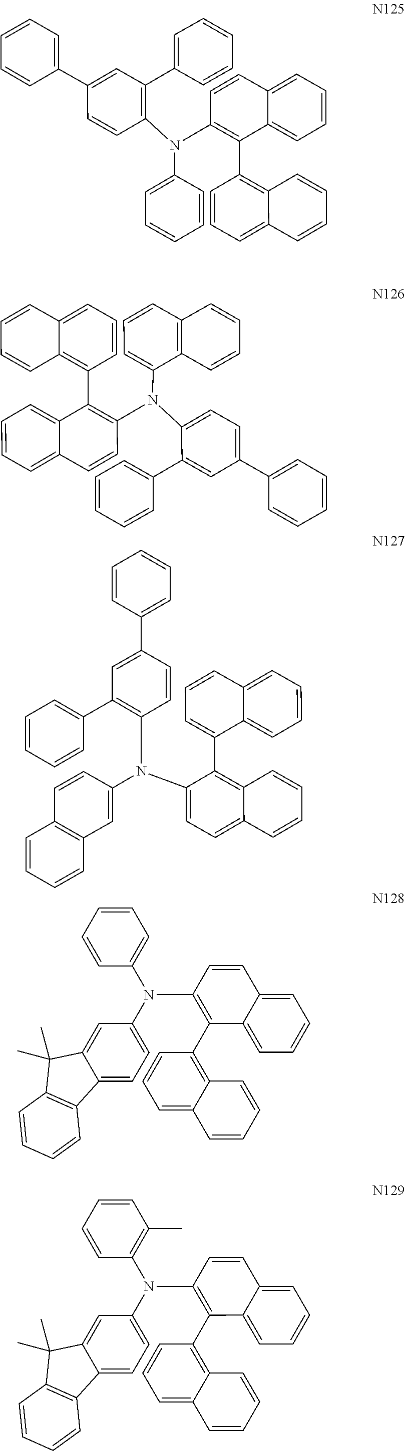

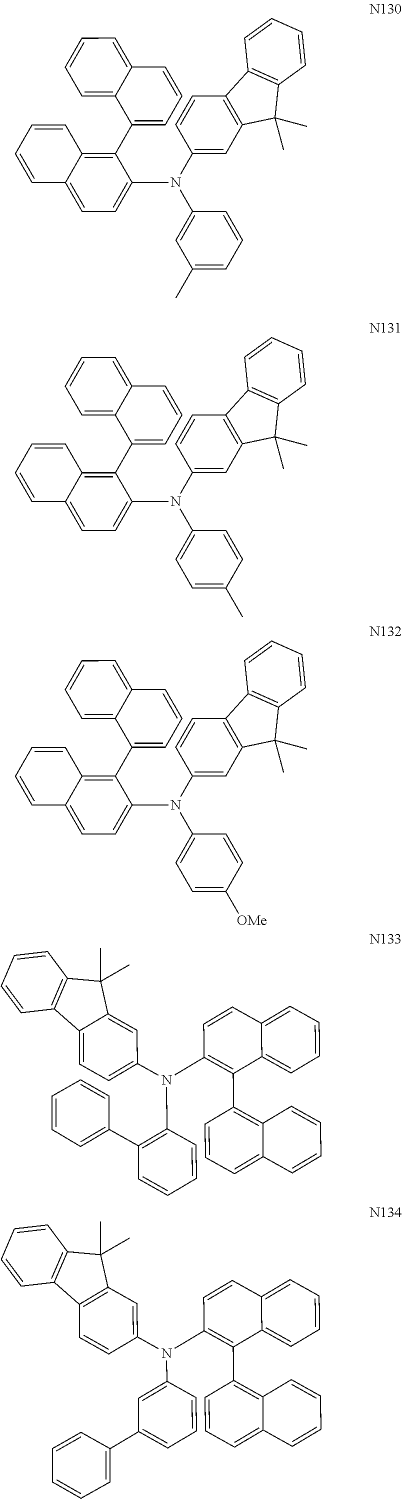

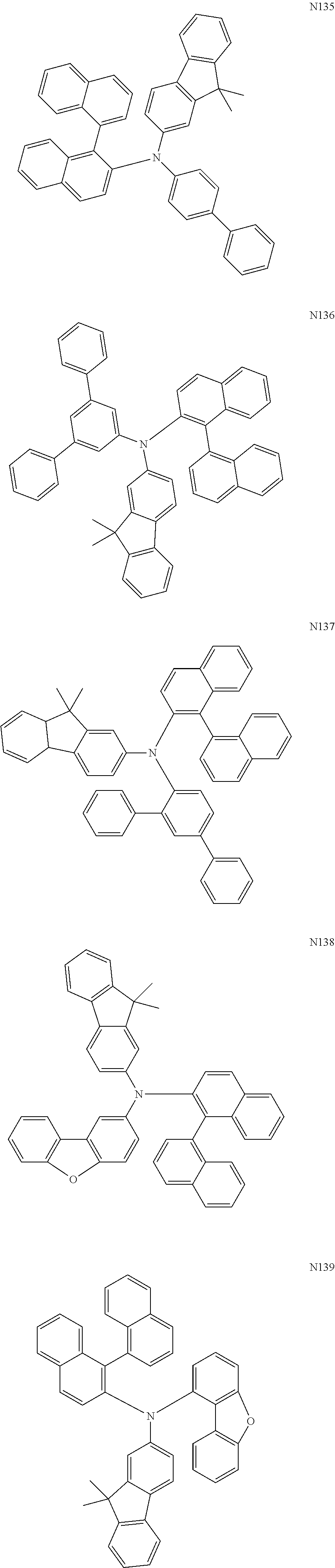 [SMILES: CC1(C)c2ccccc2-c2ccc(N(c3ccc(-c4ccccc4)cc3)c3ccc4ccccc4c3-c3cccc4ccccc34)cc21 ; CC1(C)c2ccccc2-c2ccc(N(c3cc(-c4ccccc4)cc(-c4ccccc4)c3)c3ccc4ccccc4c3-c3cccc4ccccc34)cc21 ; CC1(C)c2cc(N(c3ccc(-c4ccccc4)cc3-c3ccccc3)c3ccc4ccccc4c3-c3cccc4ccccc34)ccc2C2C=CC=CC21 ; CC1(C)c2ccccc2-c2ccc(N(c3ccc4oc5ccccc5c4c3)c3ccc4ccccc4c3-c3cccc4ccccc34)cc21 ; CC1(C)c2ccccc2-c2ccc(N(c3ccc4ccccc4c3-c3cccc4ccccc34)c3cccc4oc5ccccc5c34)cc21]

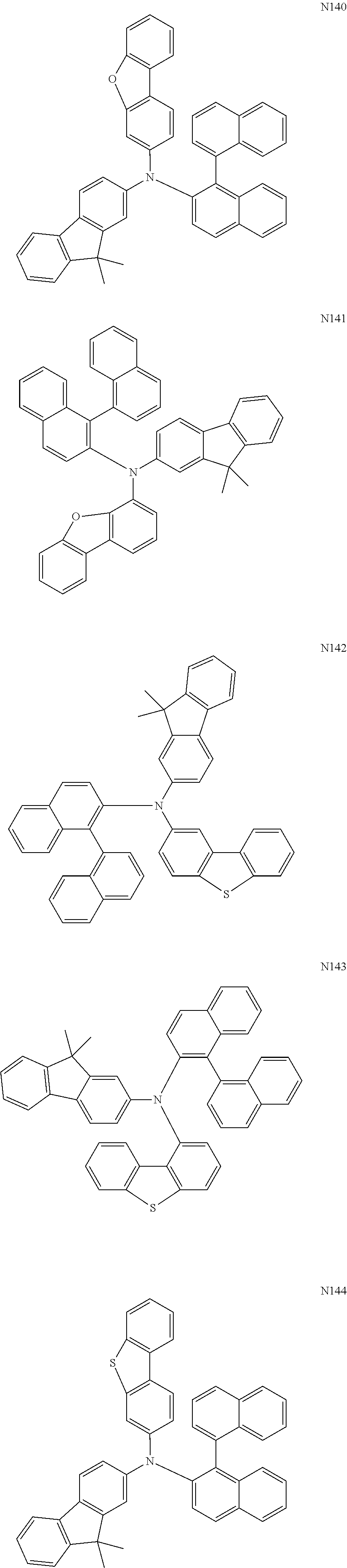 [SMILES: CC1(C)c2ccccc2-c2ccc(N(c3ccc4c(c3)oc3ccccc34)c3ccc4ccccc4c3-c3cccc4ccccc34)cc21 ; CC1(C)c2ccccc2-c2ccc(N(c3ccc4ccccc4c3-c3cccc4ccccc34)c3cccc4c3oc3ccccc34)cc21 ; CC1(C)c2ccccc2-c2ccc(N(c3ccc4sc5ccccc5c4c3)c3ccc4ccccc4c3-c3cccc4ccccc34)cc21 ; CC1(C)c2ccccc2-c2ccc(N(c3ccc4ccccc4c3-c3cccc4ccccc34)c3cccc4sc5ccccc5c34)cc21 ; CC1(C)c2ccccc2-c2ccc(N(c3ccc4c(c3)sc3ccccc34)c3ccc4ccccc4c3-c3cccc4ccccc34)cc21]

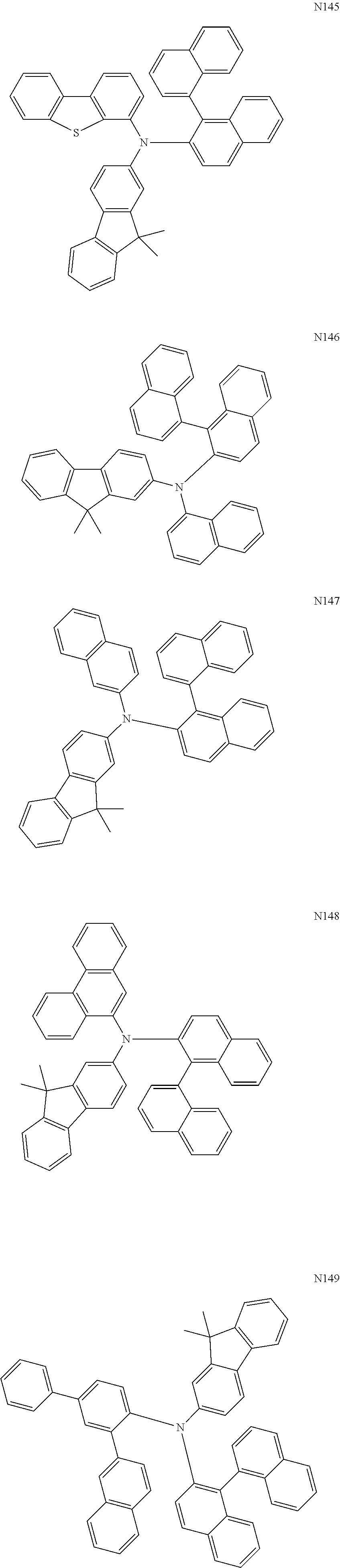 [SMILES: CC1(C)c2ccccc2-c2ccc(N(c3ccc4ccccc4c3-c3cccc4ccccc34)c3cccc4c3sc3ccccc34)cc21 ; CC1(C)c2ccccc2-c2ccc(N(c3ccc4ccccc4c3-c3cccc4ccccc34)c3cccc4ccccc34)cc21 ; CC1(C)c2ccccc2-c2ccc(N(c3ccc4ccccc4c3)c3ccc4ccccc4c3-c3cccc4ccccc34)cc21 ; CC1(C)c2ccccc2-c2ccc(N(c3ccc4ccccc4c3-c3cccc4ccccc34)c3cc4ccccc4c4ccccc34)cc21 ; CC1(C)c2ccccc2-c2ccc(N(c3ccc(-c4ccccc4)cc3-c3ccc4ccccc4c3)c3ccc4ccccc4c3-c3cccc4ccccc34)cc21]

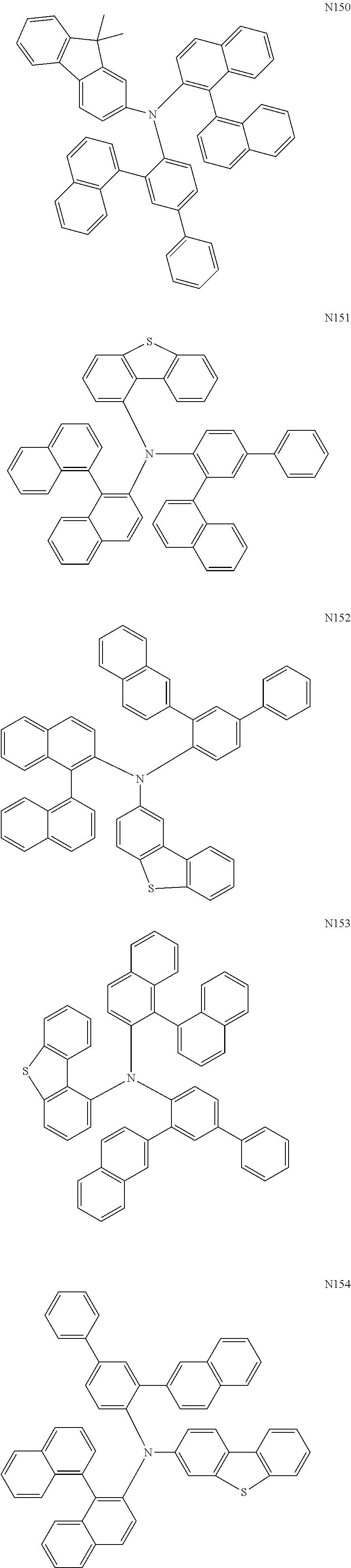 [SMILES: CC1(C)c2ccccc2-c2ccc(N(c3ccc(-c4ccccc4)cc3-c3cccc4ccccc34)c3ccc4ccccc4c3-c3cccc4ccccc34)cc21 ; c1ccc(-c2ccc(N(c3ccc4ccccc4c3-c3cccc4ccccc34)c3cccc4sc5ccccc5c34)c(-c3cccc4ccccc34)c2)cc1 ; c1ccc(-c2ccc(N(c3ccc4sc5ccccc5c4c3)c3ccc4ccccc4c3-c3cccc4ccccc34)c(-c3ccc4ccccc4c3)c2)cc1 ; c1ccc(-c2ccc(N(c3ccc4ccccc4c3-c3cccc4ccccc34)c3cccc4sc5ccccc5c34)c(-c3ccc4ccccc4c3)c2)cc1 ; c1ccc(-c2ccc(N(c3ccc4c(c3)sc3ccccc34)c3ccc4ccccc4c3-c3cccc4ccccc34)c(-c3ccc4ccccc4c3)c2)cc1]

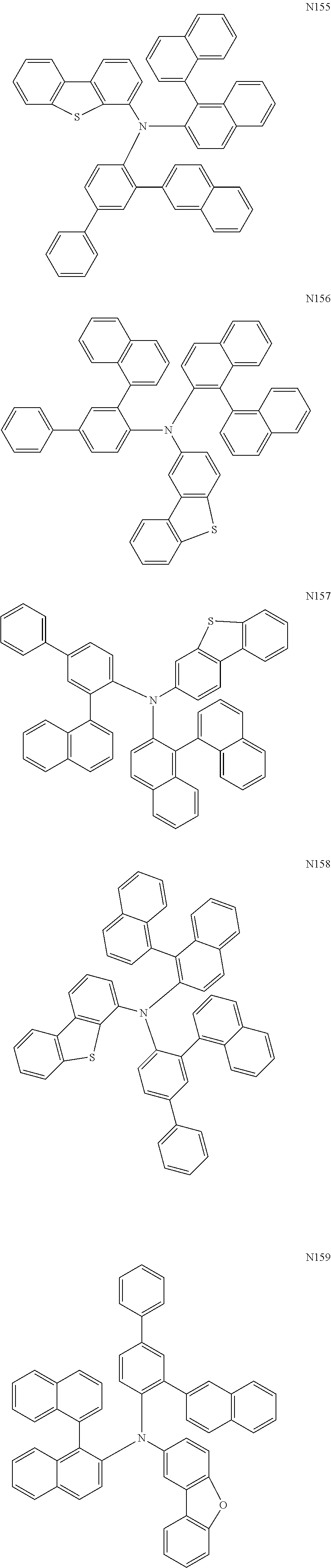 [SMILES: c1ccc(-c2ccc(N(c3ccc4ccccc4c3-c3cccc4ccccc34)c3cccc4c3sc3ccccc34)c(-c3ccc4ccccc4c3)c2)cc1 ; c1ccc(-c2ccc(N(c3ccc4sc5ccccc5c4c3)c3ccc4ccccc4c3-c3cccc4ccccc34)c(-c3cccc4ccccc34)c2)cc1 ; c1ccc(-c2ccc(N(c3ccc4c(c3)sc3ccccc34)c3ccc4ccccc4c3-c3cccc4ccccc34)c(-c3cccc4ccccc34)c2)cc1 ; c1ccc(-c2ccc(N(c3ccc4ccccc4c3-c3cccc4ccccc34)c3cccc4c3sc3ccccc34)c(-c3cccc4ccccc34)c2)cc1 ; c1ccc(-c2ccc(N(c3ccc4oc5ccccc5c4c3)c3ccc4ccccc4c3-c3cccc4ccccc34)c(-c3ccc4ccccc4c3)c2)cc1]

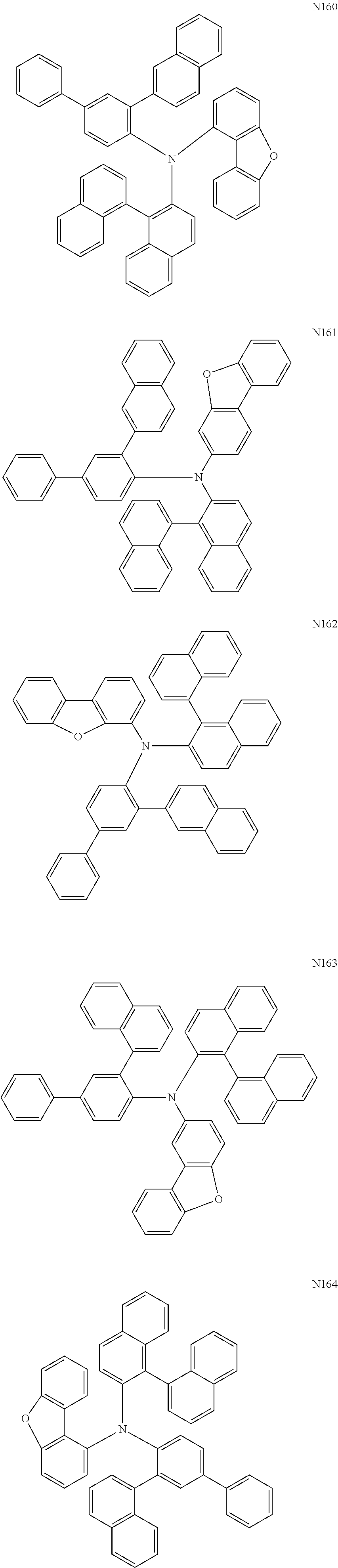 [SMILES: c1ccc(-c2ccc(N(c3ccc4ccccc4c3-c3cccc4ccccc34)c3cccc4oc5ccccc5c34)c(-c3ccc4ccccc4c3)c2)cc1 ; c1ccc(-c2ccc(N(c3ccc4c(c3)oc3ccccc34)c3ccc4ccccc4c3-c3cccc4ccccc34)c(-c3ccc4ccccc4c3)c2)cc1 ; c1ccc(-c2ccc(N(c3ccc4ccccc4c3-c3cccc4ccccc34)c3cccc4c3oc3ccccc34)c(-c3ccc4ccccc4c3)c2)cc1 ; c1ccc(-c2ccc(N(c3ccc4oc5ccccc5c4c3)c3ccc4ccccc4c3-c3cccc4ccccc34)c(-c3cccc4ccccc34)c2)cc1 ; c1ccc(-c2ccc(N(c3ccc4ccccc4c3-c3cccc4ccccc34)c3cccc4oc5ccccc5c34)c(-c3cccc4ccccc34)c2)cc1]

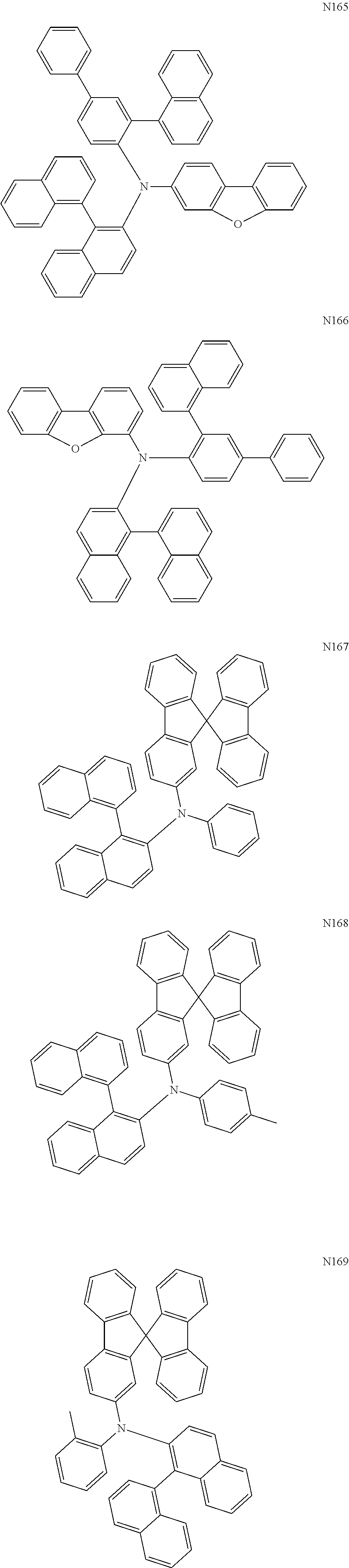 [SMILES: c1ccc(-c2ccc(N(c3ccc4c(c3)oc3ccccc34)c3ccc4ccccc4c3-c3cccc4ccccc34)c(-c3cccc4ccccc34)c2)cc1 ; c1ccc(-c2ccc(N(c3ccc4ccccc4c3-c3cccc4ccccc34)c3cccc4c3oc3ccccc34)c(-c3cccc4ccccc34)c2)cc1 ; c1ccc(N(c2ccc3c(c2)C2(c4ccccc4-c4ccccc42)c2ccccc2-3)c2ccc3ccccc3c2-c2cccc3ccccc23)cc1 ; Cc1ccc(N(c2ccc3c(c2)C2(c4ccccc4-c4ccccc42)c2ccccc2-3)c2ccc3ccccc3c2-c2cccc3ccccc23)cc1 ; Cc1ccccc1N(c1ccc2c(c1)C1(c3ccccc3-c3ccccc31)c1ccccc1-2)c1ccc2ccccc2c1-c1cccc2ccccc12]

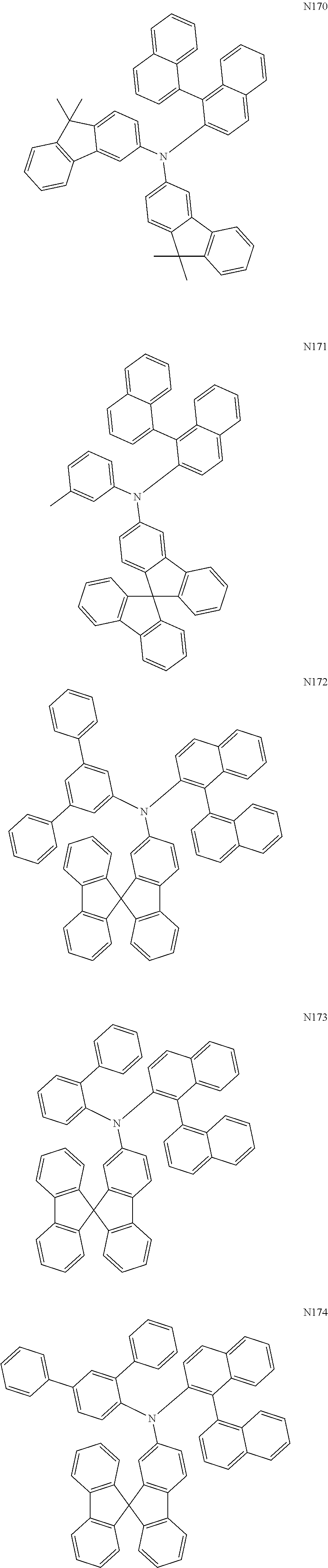 [SMILES: CC1(C)c2ccccc2-c2cc(N(c3ccc4c(c3)-c3ccccc3C4(C)C)c3ccc4ccccc4c3-c3cccc4ccccc34)ccc21 ; Cc1cccc(N(c2ccc3c(c2)-c2ccccc2C32c3ccccc3-c3ccccc32)c2ccc3ccccc3c2-c2cccc3ccccc23)c1 ; c1ccc(-c2cc(-c3ccccc3)cc(N(c3ccc4c(c3)C3(c5ccccc5-c5ccccc53)c3ccccc3-4)c3ccc4ccccc4c3-c3cccc4ccccc34)c2)cc1 ; c1ccc(-c2ccccc2N(c2ccc3c(c2)C2(c4ccccc4-c4ccccc42)c2ccccc2-3)c2ccc3ccccc3c2-c2cccc3ccccc23)cc1 ; c1ccc(-c2ccc(N(c3ccc4c(c3)C3(c5ccccc5-c5ccccc53)c3ccccc3-4)c3ccc4ccccc4c3-c3cccc4ccccc34)c(-c3ccccc3)c2)cc1]

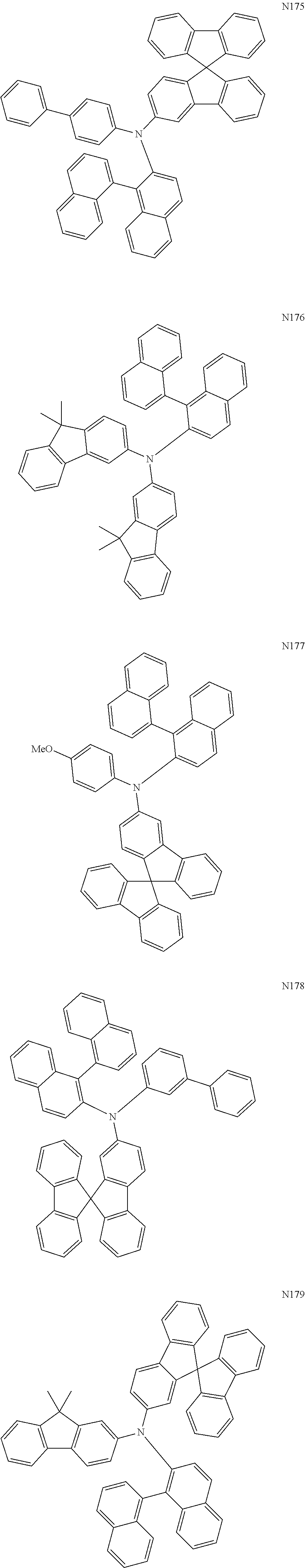 [SMILES: c1ccc(-c2ccc(N(c3ccc4c(c3)-c3ccccc3C43c4ccccc4-c4ccccc43)c3ccc4ccccc4c3-c3cccc4ccccc34)cc2)cc1 ; CC1(C)c2ccccc2-c2cc(N(c3ccc4c(c3)C(C)(C)c3ccccc3-4)c3ccc4ccccc4c3-c3cccc4ccccc34)ccc21 ; COc1ccc(N(c2ccc3c(c2)-c2ccccc2C32c3ccccc3-c3ccccc32)c2ccc3ccccc3c2-c2cccc3ccccc23)cc1 ; c1ccc(-c2cccc(N(c3ccc4c(c3)C3(c5ccccc5-c5ccccc53)c3ccccc3-4)c3ccc4ccccc4c3-c3cccc4ccccc34)c2)cc1 ; CC1(C)c2ccccc2-c2ccc(N(c3ccc4c(c3)C3(c5ccccc5-c5ccccc53)c3ccccc3-4)c3ccc4ccccc4c3-c3cccc4ccccc34)cc21]

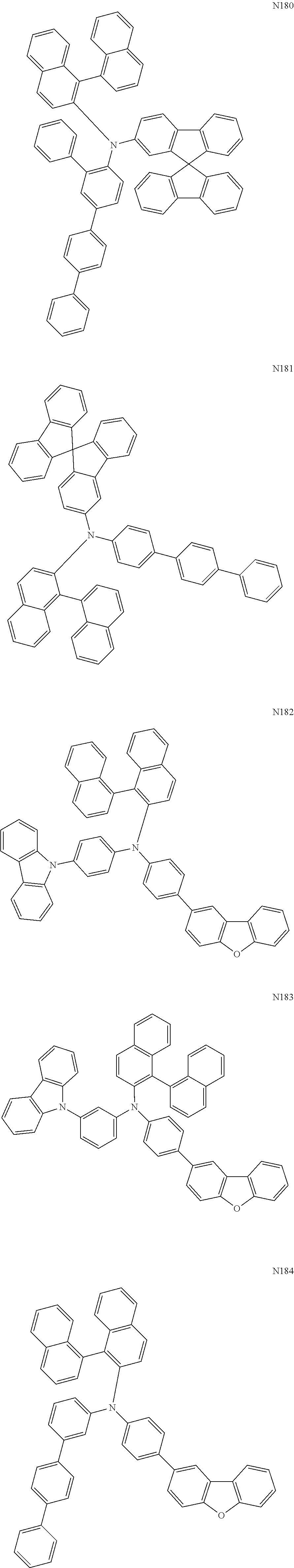

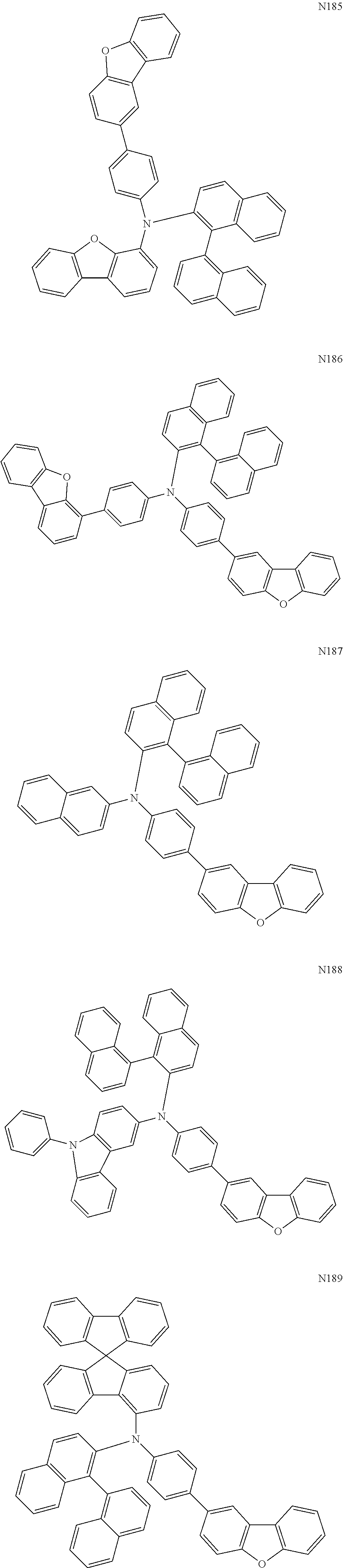 [SMILES: c1ccc2c(-c3c(N(c4ccc(-c5ccc6oc7ccccc7c6c5)cc4)c4cccc5c4oc4ccccc45)ccc4ccccc34)cccc2c1 ; c1ccc2c(-c3c(N(c4ccc(-c5ccc6oc7ccccc7c6c5)cc4)c4ccc(-c5cccc6c5oc5ccccc56)cc4)ccc4ccccc34)cccc2c1 ; c1ccc2cc(N(c3ccc(-c4ccc5oc6ccccc6c5c4)cc3)c3ccc4ccccc4c3-c3cccc4ccccc34)ccc2c1 ; c1ccc(-n2c3ccccc3c3cc(N(c4ccc(-c5ccc6oc7ccccc7c6c5)cc4)c4ccc5ccccc5c4-c4cccc5ccccc45)ccc32)cc1 ; c1ccc2c(c1)-c1ccccc1C21c2ccccc2-c2c(N(c3ccc(-c4ccc5oc6ccccc6c5c4)cc3)c3ccc4ccccc4c3-c3cccc4ccccc34)cccc21]

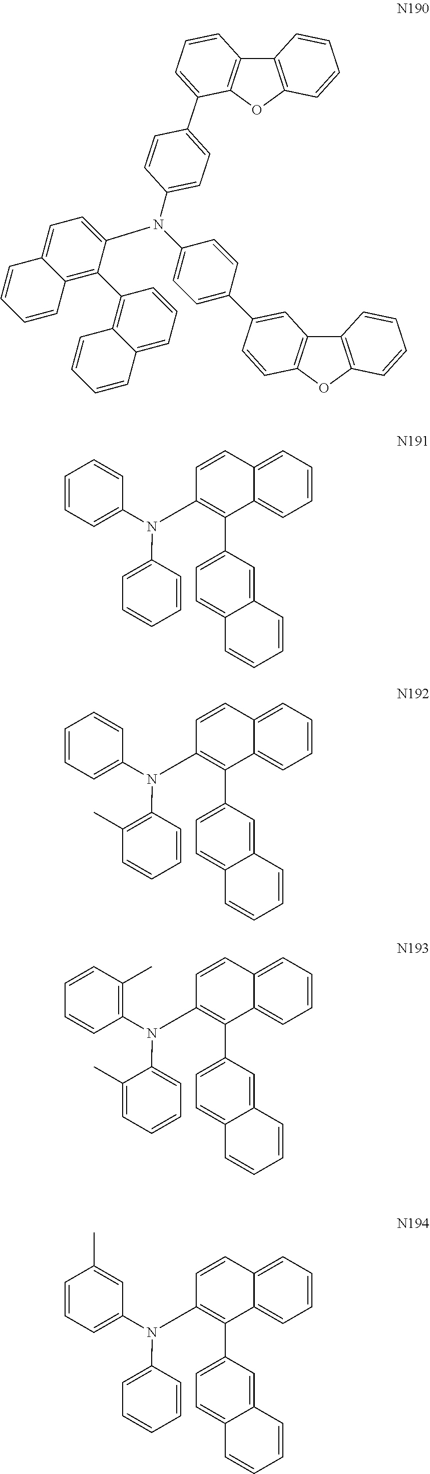

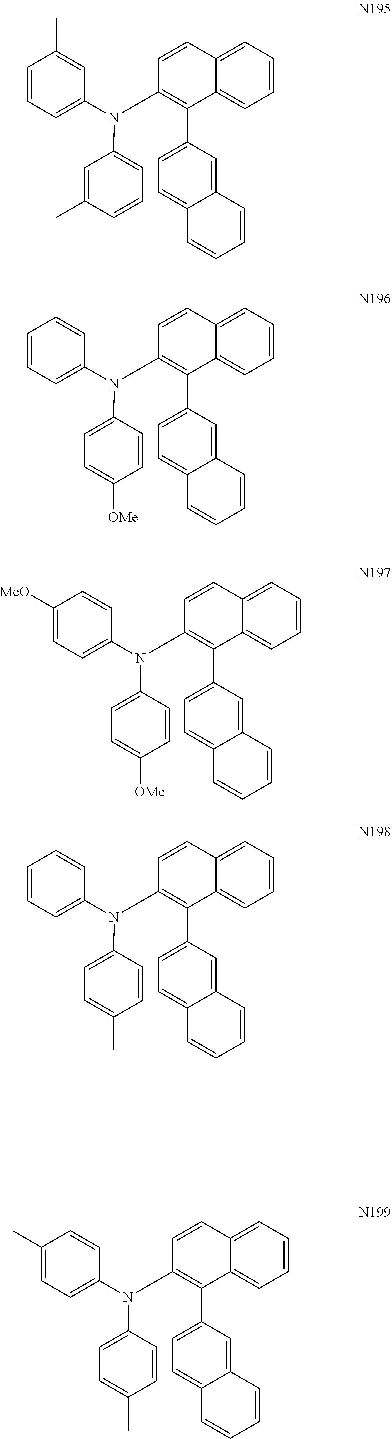

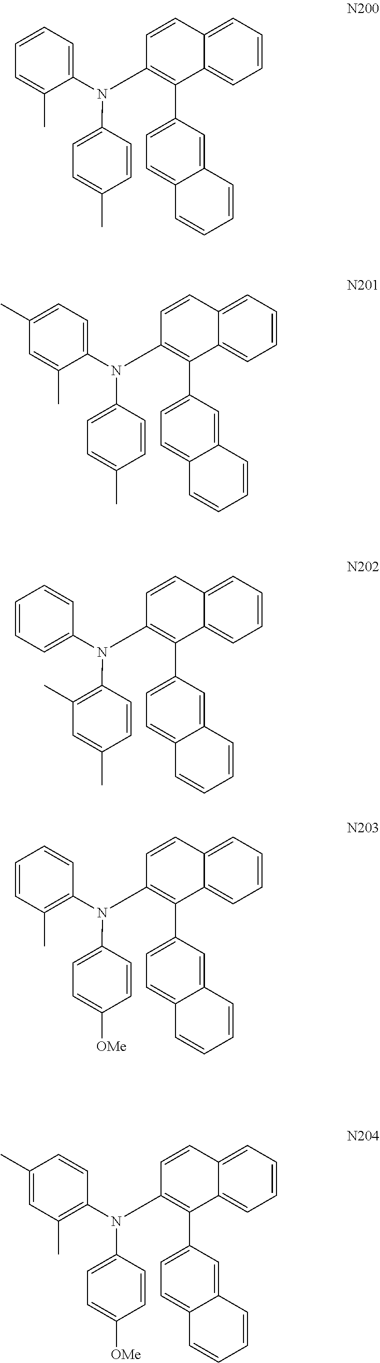 [SMILES: Cc1ccc(N(c2ccccc2C)c2ccc3ccccc3c2-c2ccc3ccccc3c2)cc1 ; Cc1ccc(N(c2ccc(C)cc2C)c2ccc3ccccc3c2-c2ccc3ccccc3c2)cc1 ; Cc1ccc(N(c2ccccc2)c2ccc3ccccc3c2-c2ccc3ccccc3c2)c(C)c1 ; COc1ccc(N(c2ccccc2C)c2ccc3ccccc3c2-c2ccc3ccccc3c2)cc1 ; COc1ccc(N(c2ccc(C)cc2C)c2ccc3ccccc3c2-c2ccc3ccccc3c2)cc1]

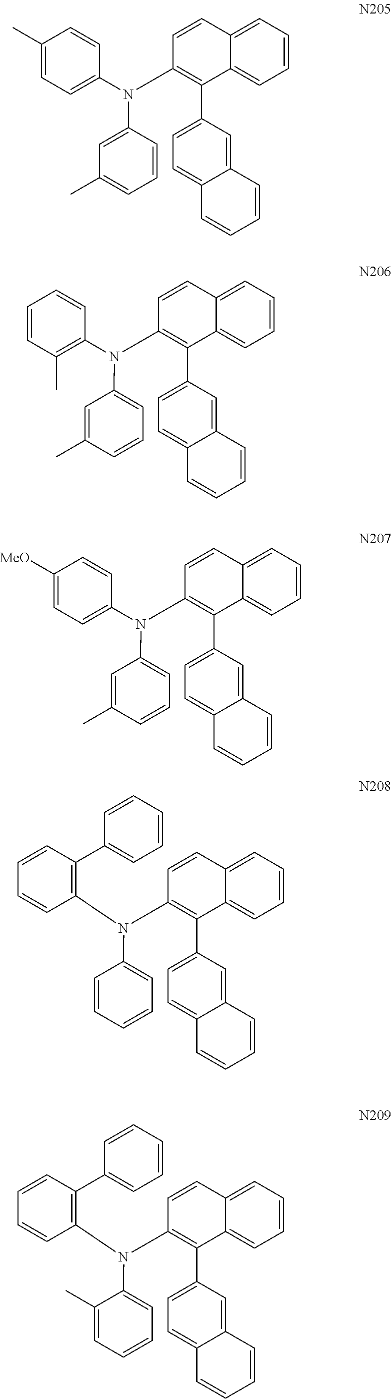 [SMILES: Cc1ccc(N(c2cccc(C)c2)c2ccc3ccccc3c2-c2ccc3ccccc3c2)cc1 ; Cc1cccc(N(c2ccccc2C)c2ccc3ccccc3c2-c2ccc3ccccc3c2)c1 ; COc1ccc(N(c2cccc(C)c2)c2ccc3ccccc3c2-c2ccc3ccccc3c2)cc1 ; c1ccc(-c2ccccc2N(c2ccccc2)c2ccc3ccccc3c2-c2ccc3ccccc3c2)cc1 ; Cc1ccccc1N(c1ccccc1-c1ccccc1)c1ccc2ccccc2c1-c1ccc2ccccc2c1]

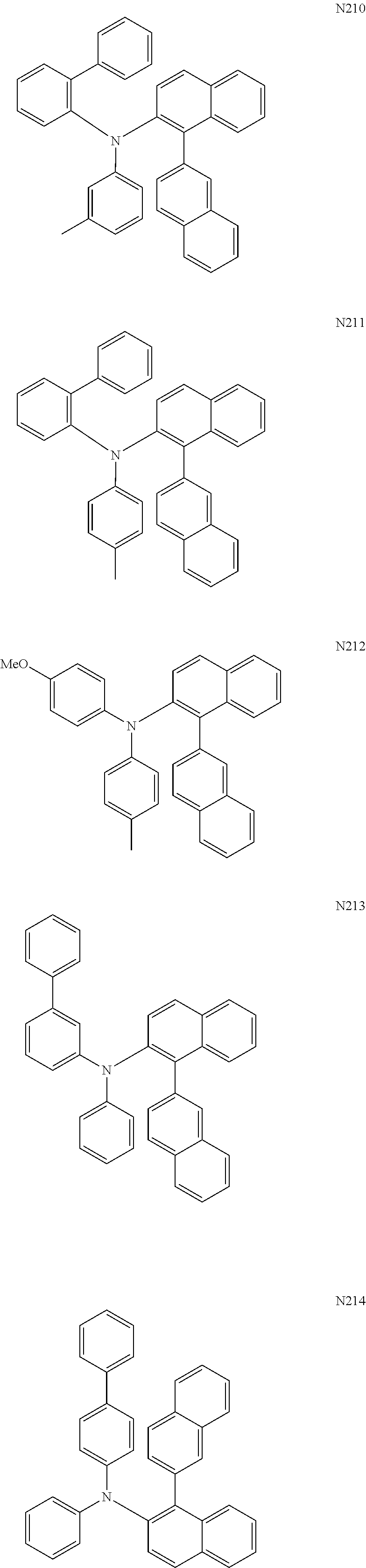 [SMILES: Cc1cccc(N(c2ccccc2-c2ccccc2)c2ccc3ccccc3c2-c2ccc3ccccc3c2)c1 ; Cc1ccc(N(c2ccccc2-c2ccccc2)c2ccc3ccccc3c2-c2ccc3ccccc3c2)cc1 ; COc1ccc(N(c2ccc(C)cc2)c2ccc3ccccc3c2-c2ccc3ccccc3c2)cc1 ; c1ccc(-c2cccc(N(c3ccccc3)c3ccc4ccccc4c3-c3ccc4ccccc4c3)c2)cc1 ; c1ccc(-c2ccc(N(c3ccccc3)c3ccc4ccccc4c3-c3ccc4ccccc4c3)cc2)cc1]

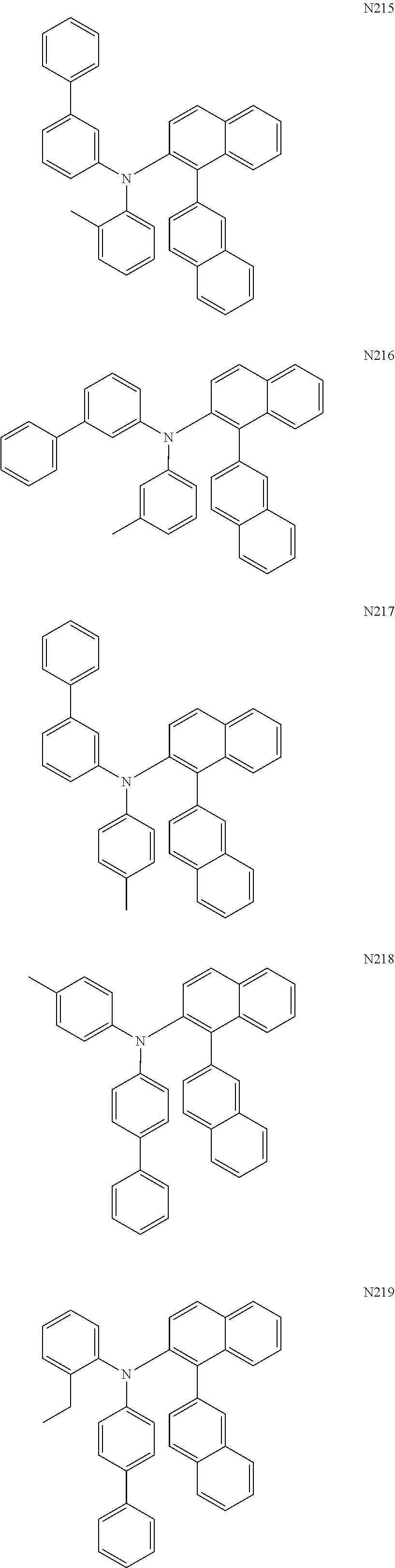 [SMILES: Cc1ccccc1N(c1cccc(-c2ccccc2)c1)c1ccc2ccccc2c1-c1ccc2ccccc2c1 ; Cc1cccc(N(c2cccc(-c3ccccc3)c2)c2ccc3ccccc3c2-c2ccc3ccccc3c2)c1 ; Cc1ccc(N(c2cccc(-c3ccccc3)c2)c2ccc3ccccc3c2-c2ccc3ccccc3c2)cc1 ; Cc1ccc(N(c2ccc(-c3ccccc3)cc2)c2ccc3ccccc3c2-c2ccc3ccccc3c2)cc1 ; CCc1ccccc1N(c1ccc(-c2ccccc2)cc1)c1ccc2ccccc2c1-c1ccc2ccccc2c1]

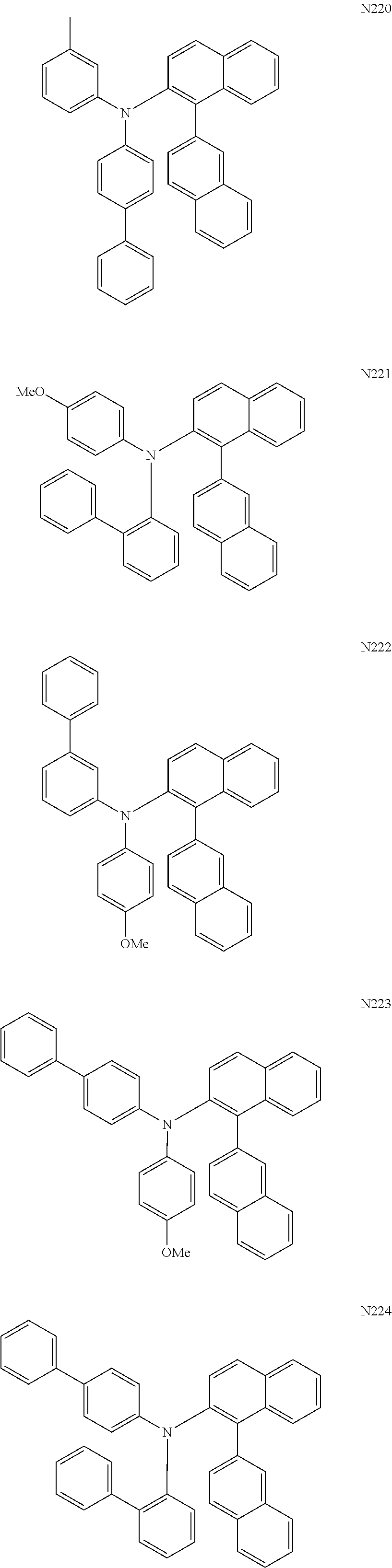 [SMILES: Cc1cccc(N(c2ccc(-c3ccccc3)cc2)c2ccc3ccccc3c2-c2ccc3ccccc3c2)c1 ; COc1ccc(N(c2ccccc2-c2ccccc2)c2ccc3ccccc3c2-c2ccc3ccccc3c2)cc1 ; COc1ccc(N(c2cccc(-c3ccccc3)c2)c2ccc3ccccc3c2-c2ccc3ccccc3c2)cc1 ; COc1ccc(N(c2ccc(-c3ccccc3)cc2)c2ccc3ccccc3c2-c2ccc3ccccc3c2)cc1 ; c1ccc(-c2ccc(N(c3ccccc3-c3ccccc3)c3ccc4ccccc4c3-c3ccc4ccccc4c3)cc2)cc1]

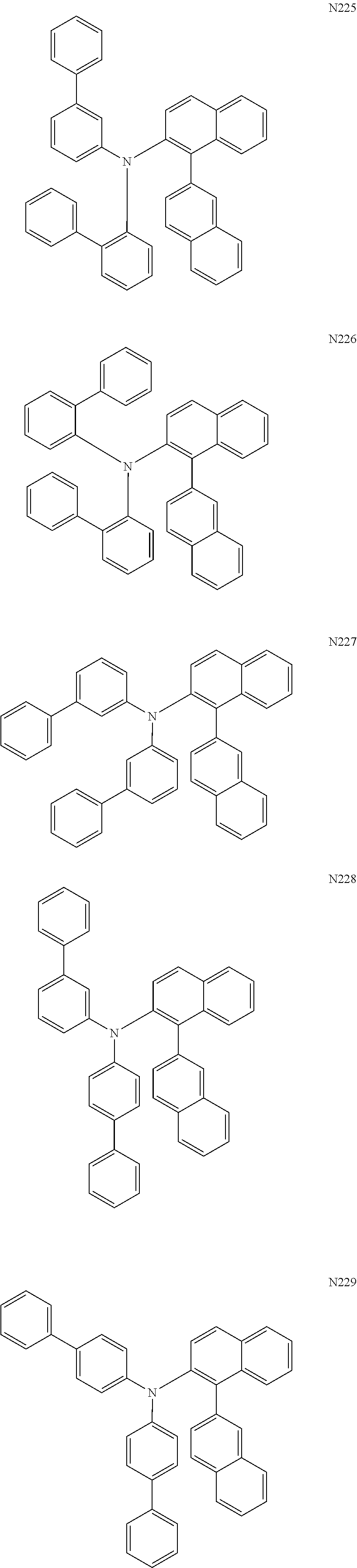 [SMILES: c1ccc(-c2cccc(N(c3ccccc3-c3ccccc3)c3ccc4ccccc4c3-c3ccc4ccccc4c3)c2)cc1 ; c1ccc(-c2ccccc2N(c2ccccc2-c2ccccc2)c2ccc3ccccc3c2-c2ccc3ccccc3c2)cc1 ; c1ccc(-c2cccc(N(c3cccc(-c4ccccc4)c3)c3ccc4ccccc4c3-c3ccc4ccccc4c3)c2)cc1 ; c1ccc(-c2ccc(N(c3cccc(-c4ccccc4)c3)c3ccc4ccccc4c3-c3ccc4ccccc4c3)cc2)cc1 ; c1ccc(-c2ccc(N(c3ccc(-c4ccccc4)cc3)c3ccc4ccccc4c3-c3ccc4ccccc4c3)cc2)cc1]

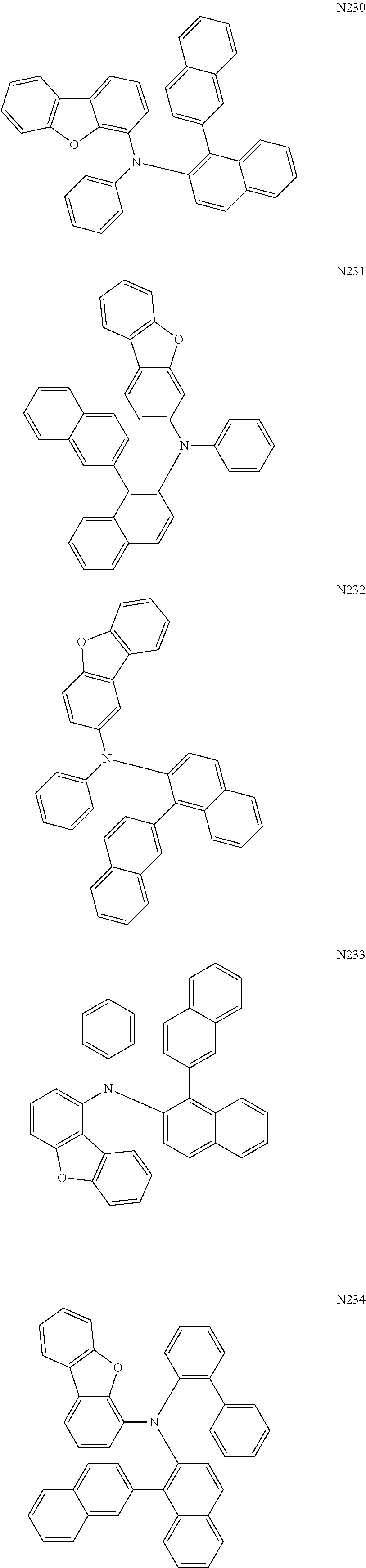

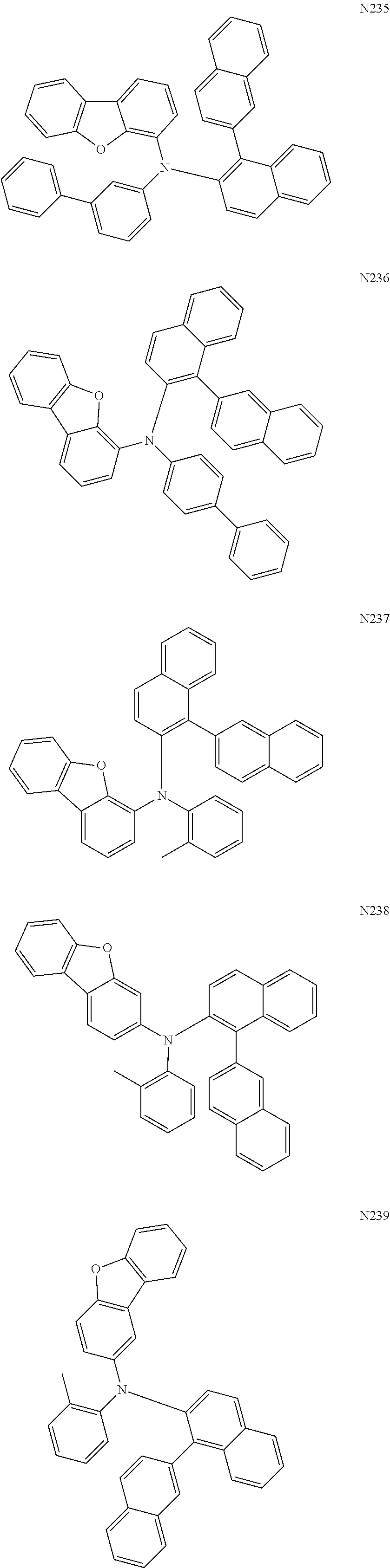 [SMILES: c1ccc(-c2cccc(N(c3ccc4ccccc4c3-c3ccc4ccccc4c3)c3cccc4c3oc3ccccc34)c2)cc1 ; c1ccc(-c2ccc(N(c3ccc4ccccc4c3-c3ccc4ccccc4c3)c3cccc4c3oc3ccccc34)cc2)cc1 ; Cc1ccccc1N(c1ccc2ccccc2c1-c1ccc2ccccc2c1)c1cccc2c1oc1ccccc12 ; Cc1ccccc1N(c1ccc2c(c1)oc1ccccc12)c1ccc2ccccc2c1-c1ccc2ccccc2c1 ; Cc1ccccc1N(c1ccc2oc3ccccc3c2c1)c1ccc2ccccc2c1-c1ccc2ccccc2c1]

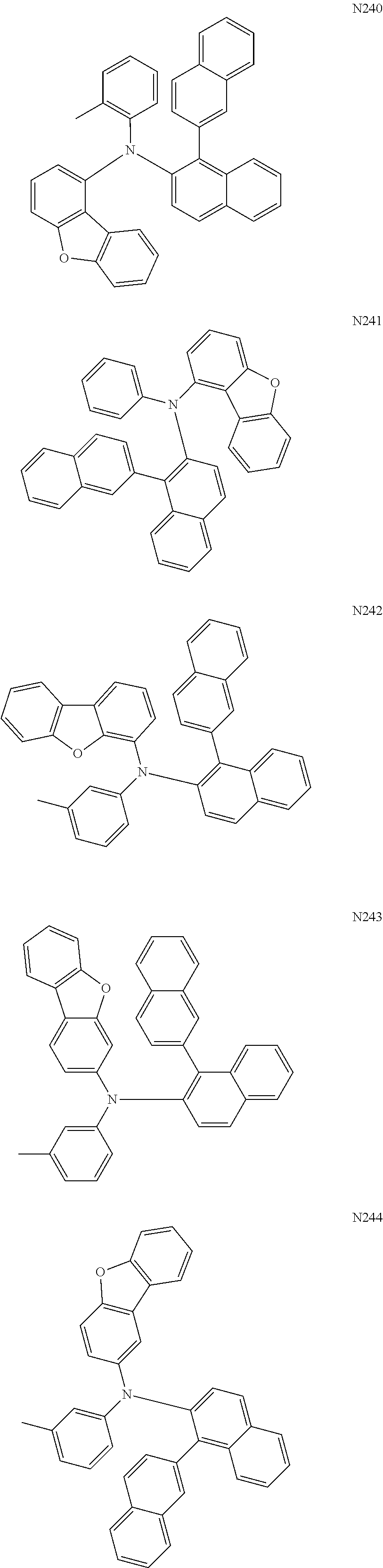 [SMILES: Cc1ccccc1N(c1ccc2ccccc2c1-c1ccc2ccccc2c1)c1cccc2oc3ccccc3c12 ; c1ccc(N(c2ccc3ccccc3c2-c2ccc3ccccc3c2)c2cccc3oc4ccccc4c23)cc1 ; Cc1cccc(N(c2ccc3ccccc3c2-c2ccc3ccccc3c2)c2cccc3c2oc2ccccc23)c1 ; Cc1cccc(N(c2ccc3c(c2)oc2ccccc23)c2ccc3ccccc3c2-c2ccc3ccccc3c2)c1 ; Cc1cccc(N(c2ccc3oc4ccccc4c3c2)c2ccc3ccccc3c2-c2ccc3ccccc3c2)c1]

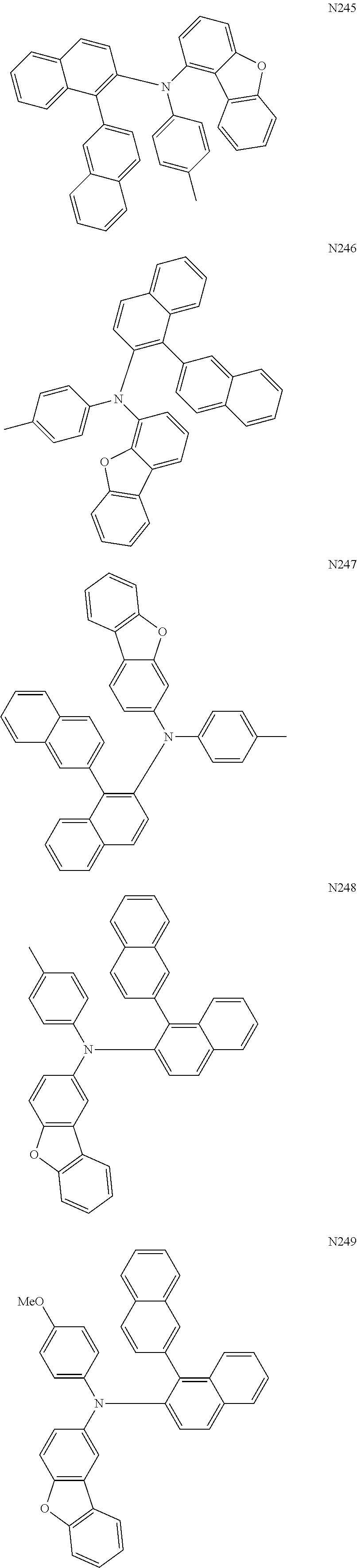

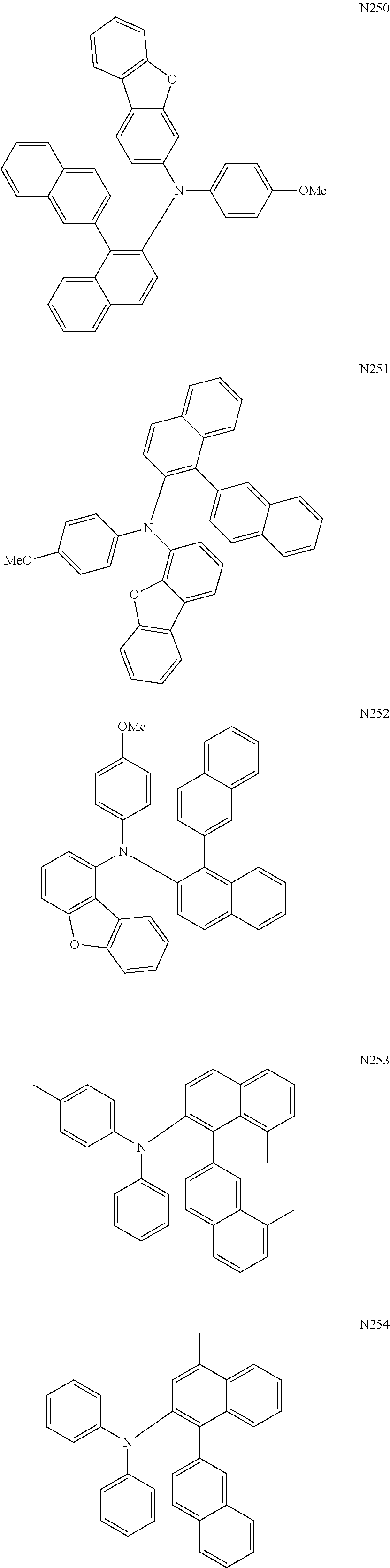

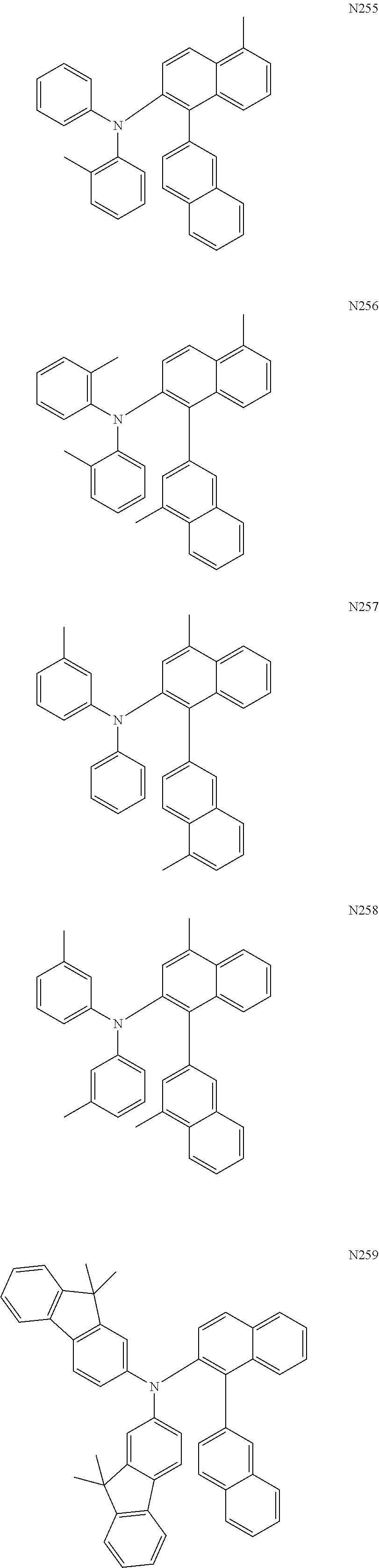 [SMILES: Cc1ccccc1N(c1ccccc1)c1ccc2c(C)cccc2c1-c1ccc2ccccc2c1 ; Cc1ccccc1N(c1ccccc1C)c1ccc2c(C)cccc2c1-c1cc(C)c2ccccc2c1 ; Cc1cccc(N(c2ccccc2)c2cc(C)c3ccccc3c2-c2ccc3c(C)cccc3c2)c1 ; Cc1cccc(N(c2cccc(C)c2)c2cc(C)c3ccccc3c2-c2cc(C)c3ccccc3c2)c1 ; CC1(C)c2ccccc2-c2ccc(N(c3ccc4c(c3)C(C)(C)c3ccccc3-4)c3ccc4ccccc4c3-c3ccc4ccccc4c3)cc21]

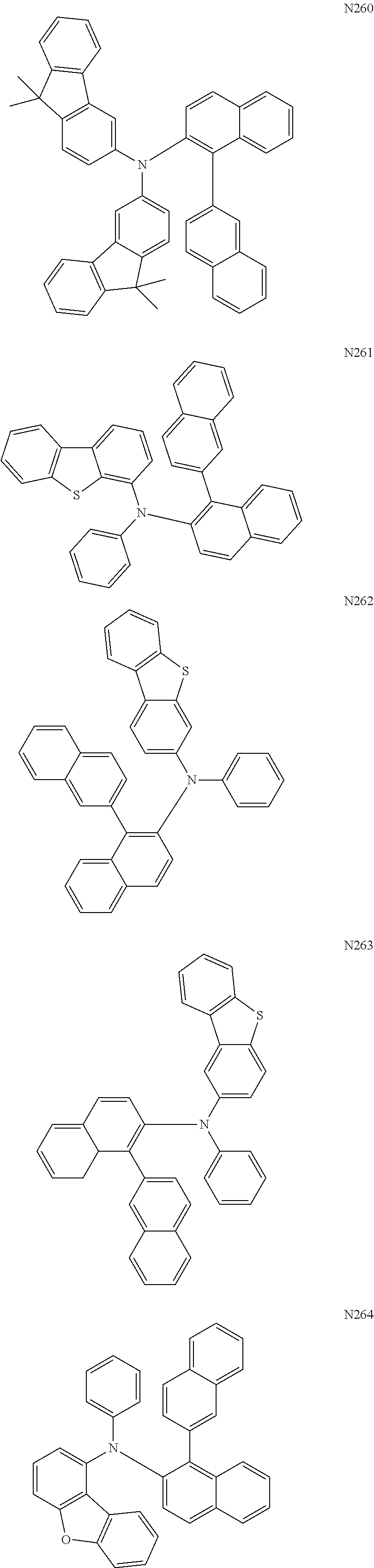

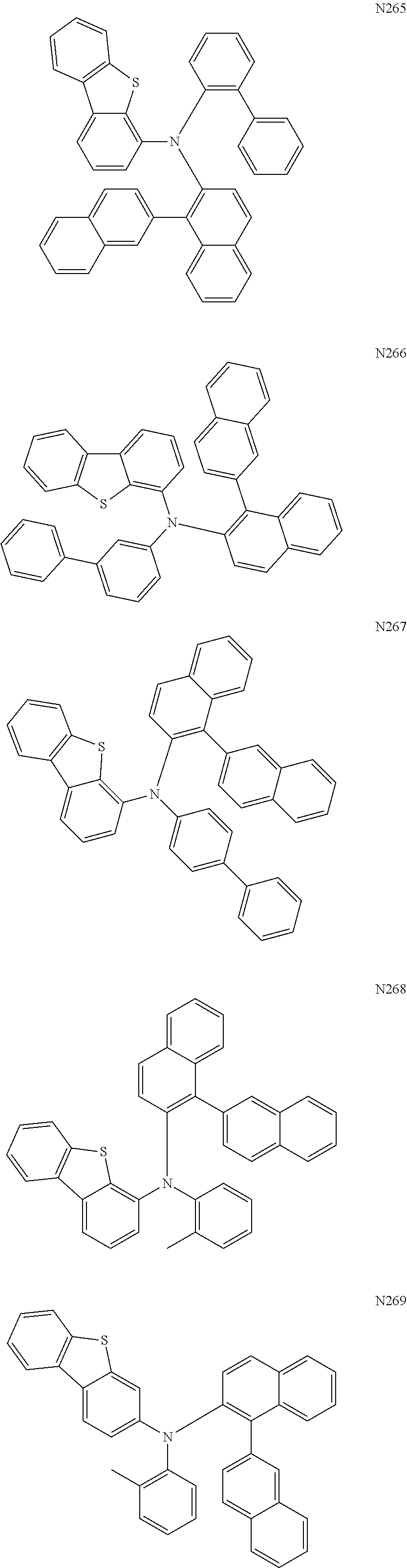 [SMILES: c1ccc(-c2ccccc2N(c2ccc3ccccc3c2-c2ccc3ccccc3c2)c2cccc3c2sc2ccccc23)cc1 ; c1ccc(-c2cccc(N(c3ccc4ccccc4c3-c3ccc4ccccc4c3)c3cccc4c3sc3ccccc34)c2)cc1 ; c1ccc(-c2ccc(N(c3ccc4ccccc4c3-c3ccc4ccccc4c3)c3cccc4c3sc3ccccc34)cc2)cc1 ; Cc1ccccc1N(c1ccc2ccccc2c1-c1ccc2ccccc2c1)c1cccc2c1sc1ccccc12 ; Cc1ccccc1N(c1ccc2c(c1)sc1ccccc12)c1ccc2ccccc2c1-c1ccc2ccccc2c1]

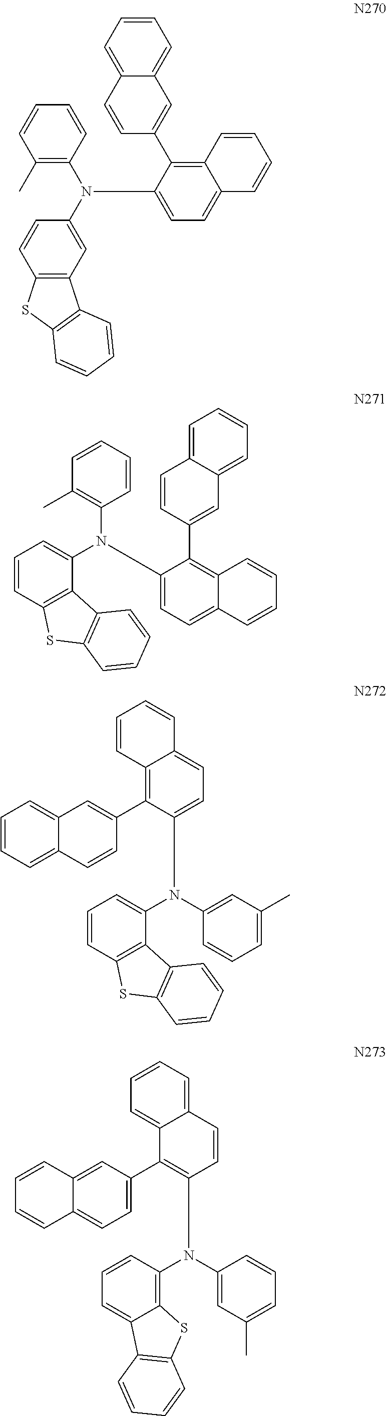

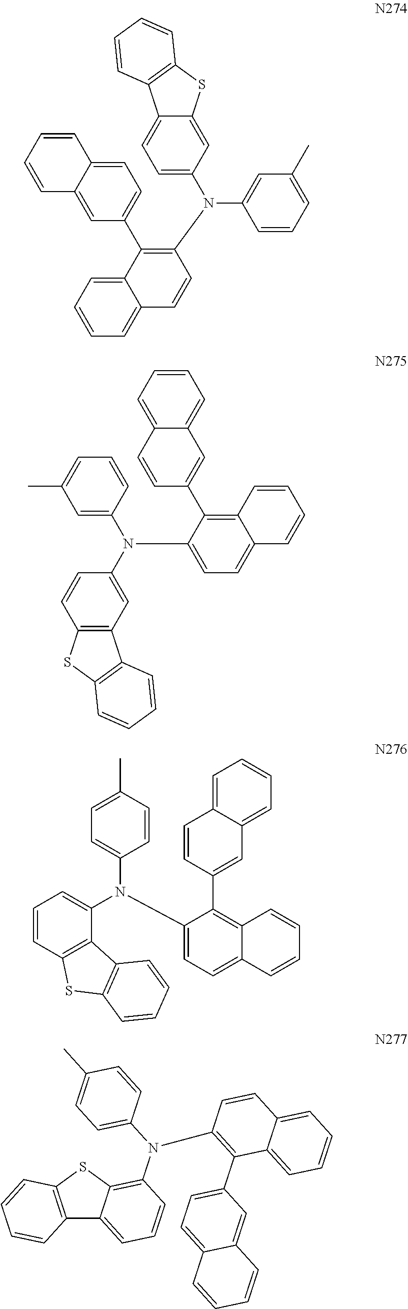

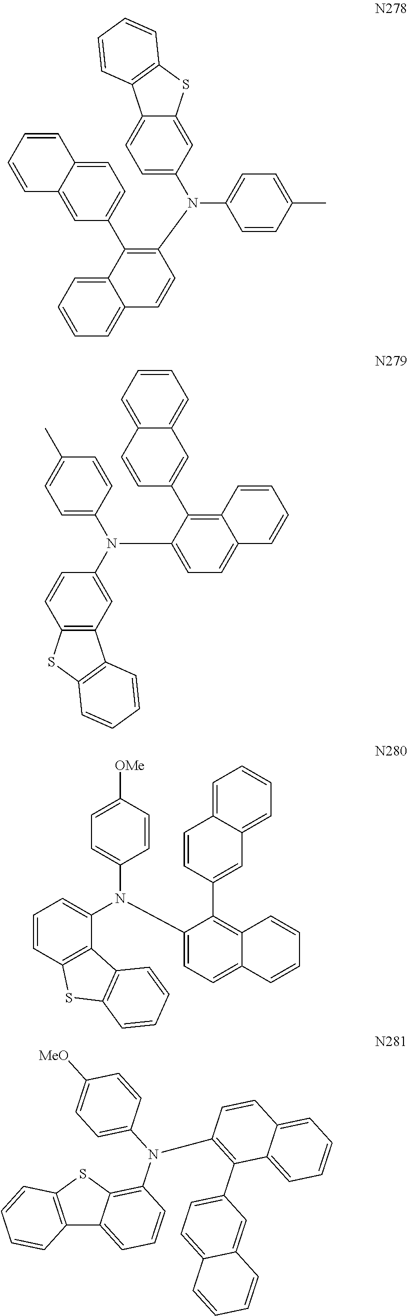

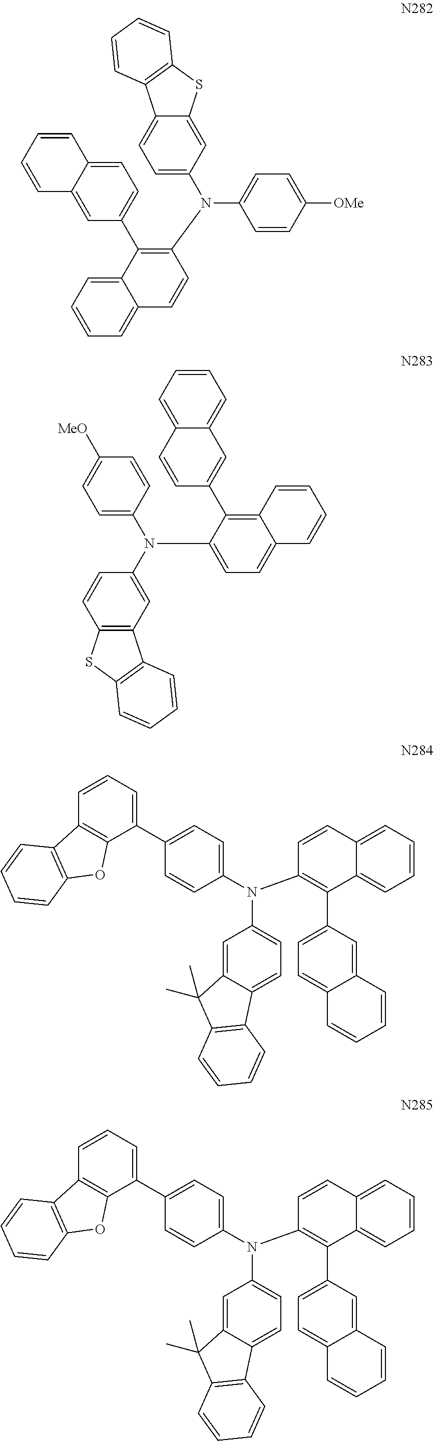 [SMILES: COc1ccc(N(c2ccc3c(c2)sc2ccccc23)c2ccc3ccccc3c2-c2ccc3ccccc3c2)cc1 ; COc1ccc(N(c2ccc3sc4ccccc4c3c2)c2ccc3ccccc3c2-c2ccc3ccccc3c2)cc1 ; CC1(C)c2ccccc2-c2ccc(N(c3ccc(-c4cccc5c4oc4ccccc45)cc3)c3ccc4ccccc4c3-c3ccc4ccccc4c3)cc21 ; CC1(C)c2ccccc2-c2ccc(N(c3ccc(-c4cccc5c4oc4ccccc45)cc3)c3ccc4ccccc4c3-c3ccc4ccccc4c3)cc21]

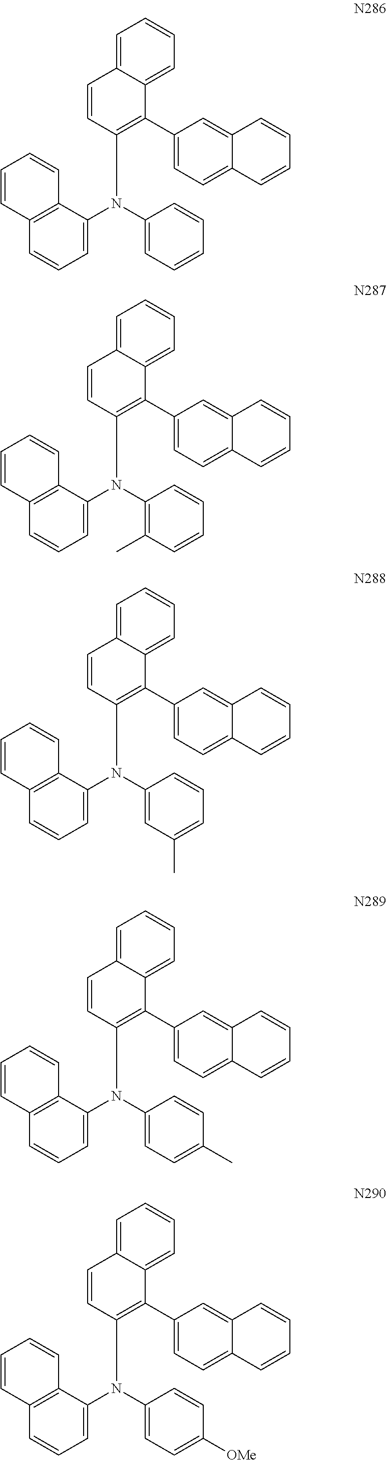

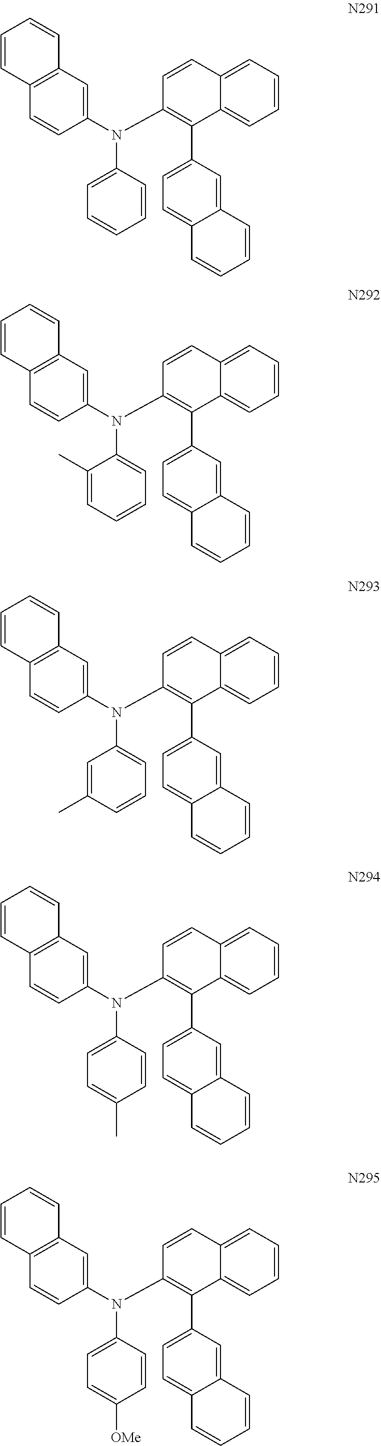 [SMILES: c1ccc(N(c2ccc3ccccc3c2)c2ccc3ccccc3c2-c2ccc3ccccc3c2)cc1 ; Cc1ccccc1N(c1ccc2ccccc2c1)c1ccc2ccccc2c1-c1ccc2ccccc2c1 ; Cc1cccc(N(c2ccc3ccccc3c2)c2ccc3ccccc3c2-c2ccc3ccccc3c2)c1 ; Cc1ccc(N(c2ccc3ccccc3c2)c2ccc3ccccc3c2-c2ccc3ccccc3c2)cc1 ; COc1ccc(N(c2ccc3ccccc3c2)c2ccc3ccccc3c2-c2ccc3ccccc3c2)cc1]

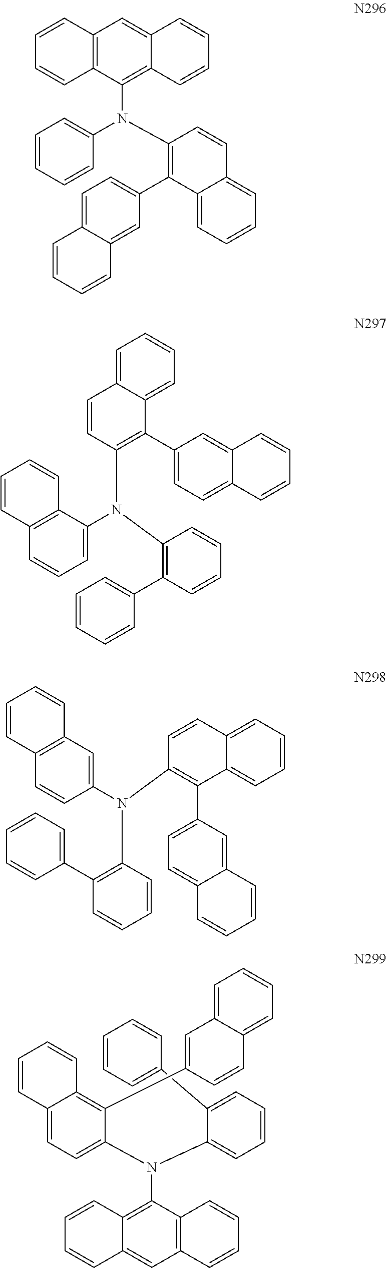

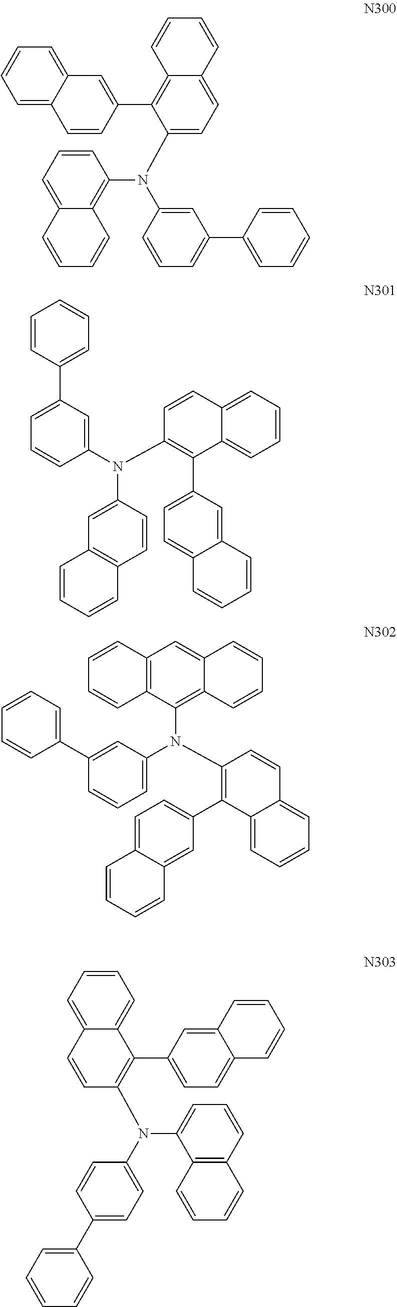

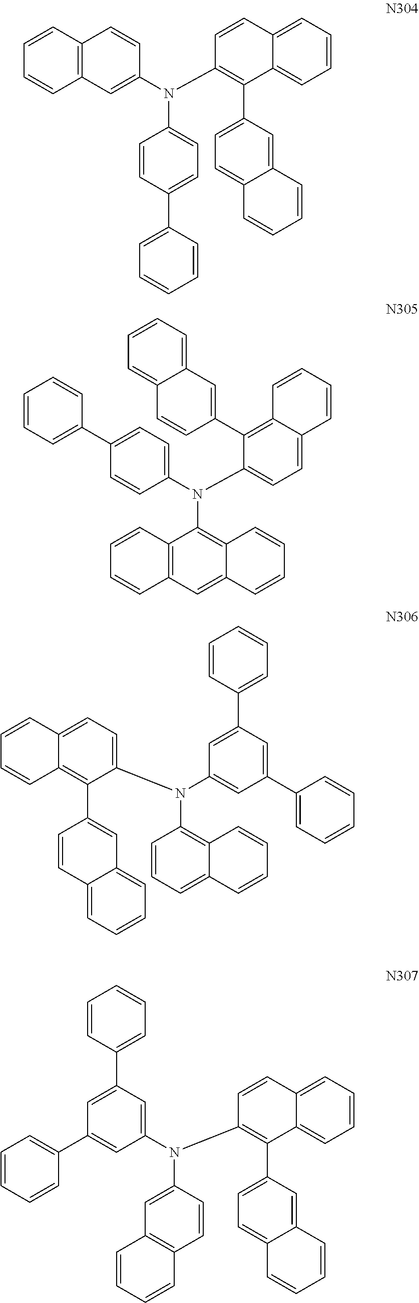

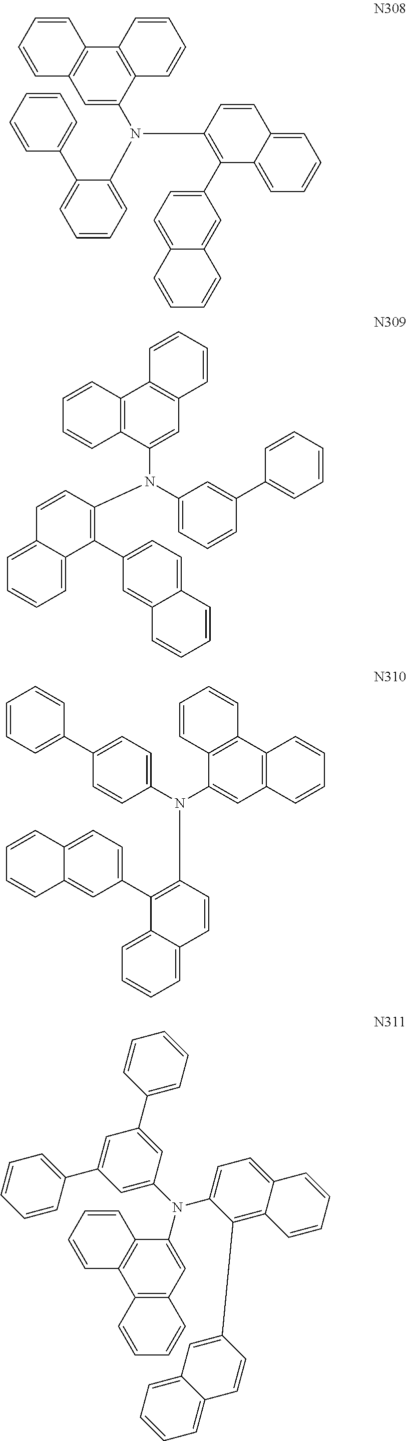

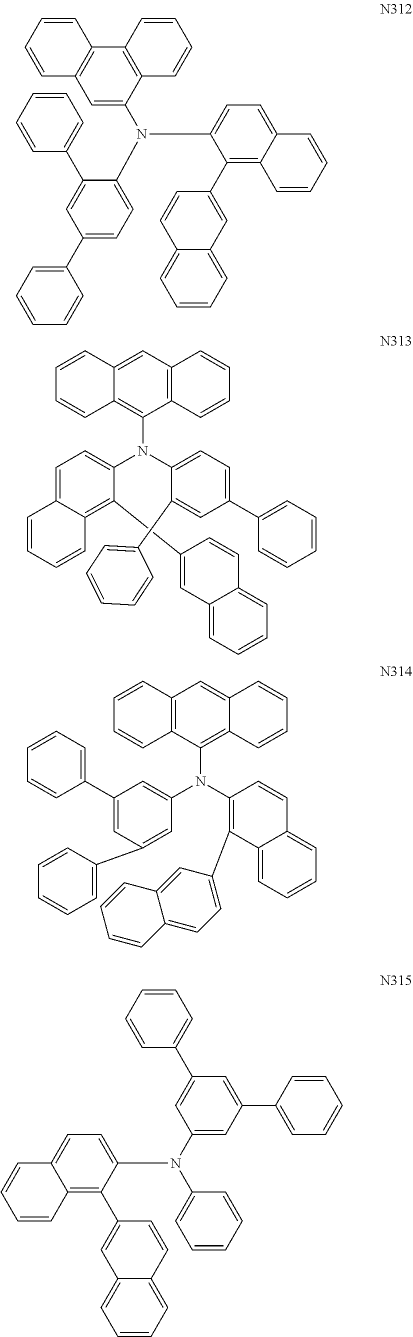

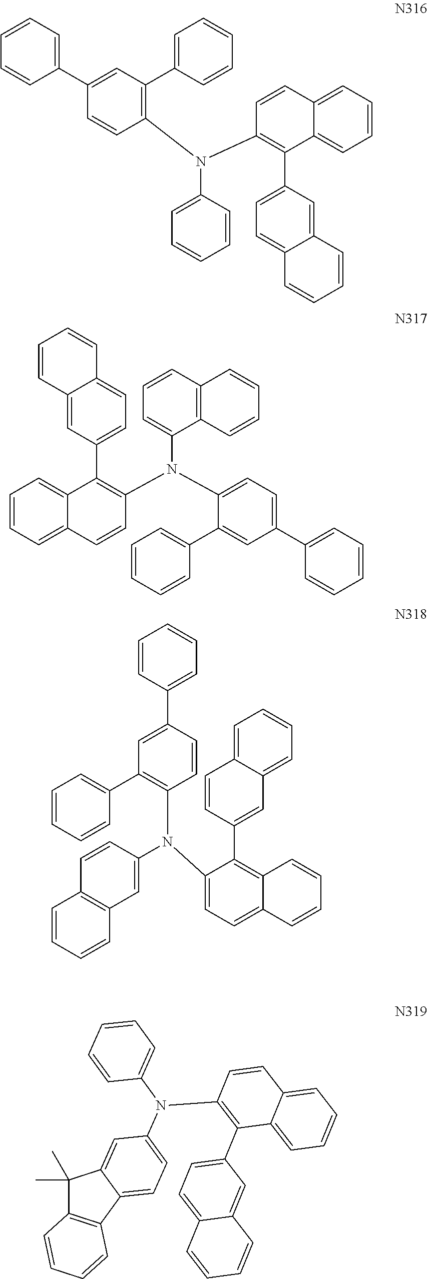

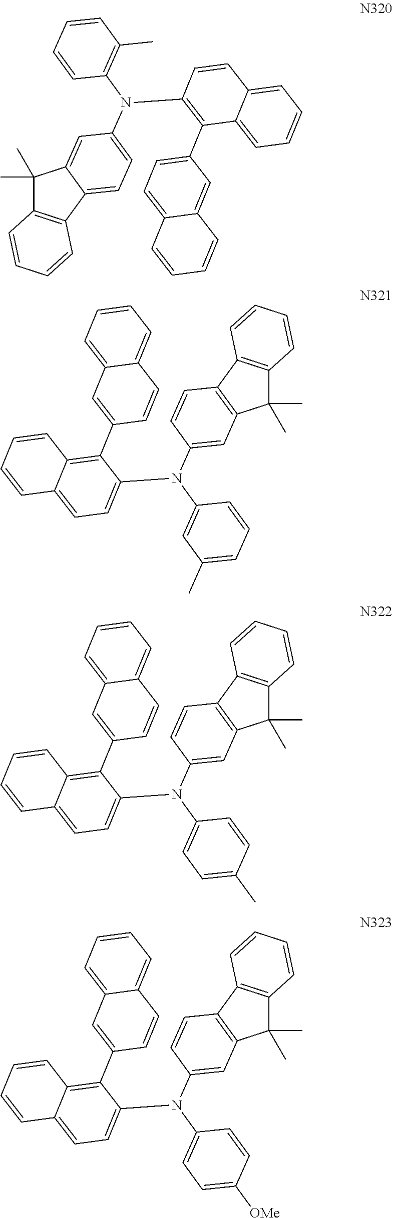

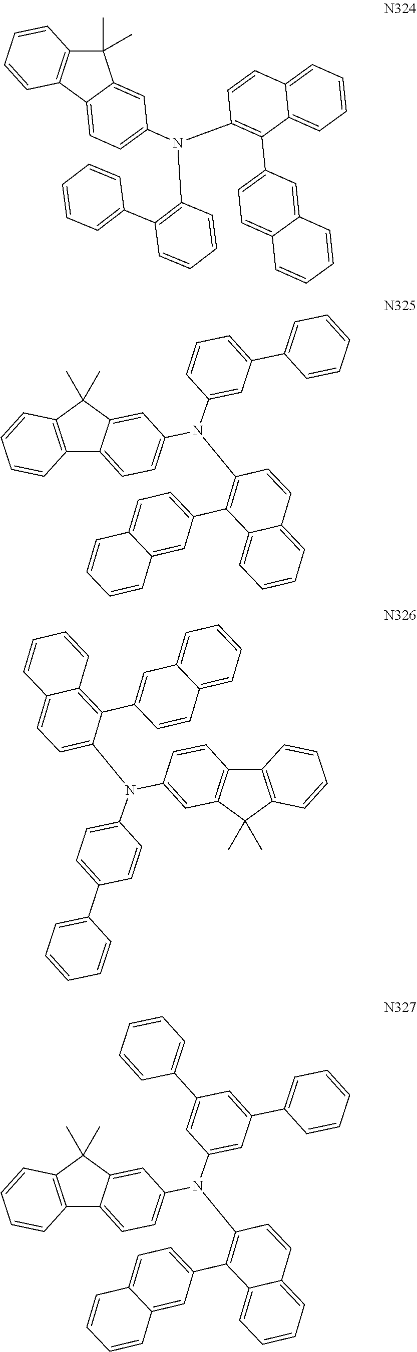

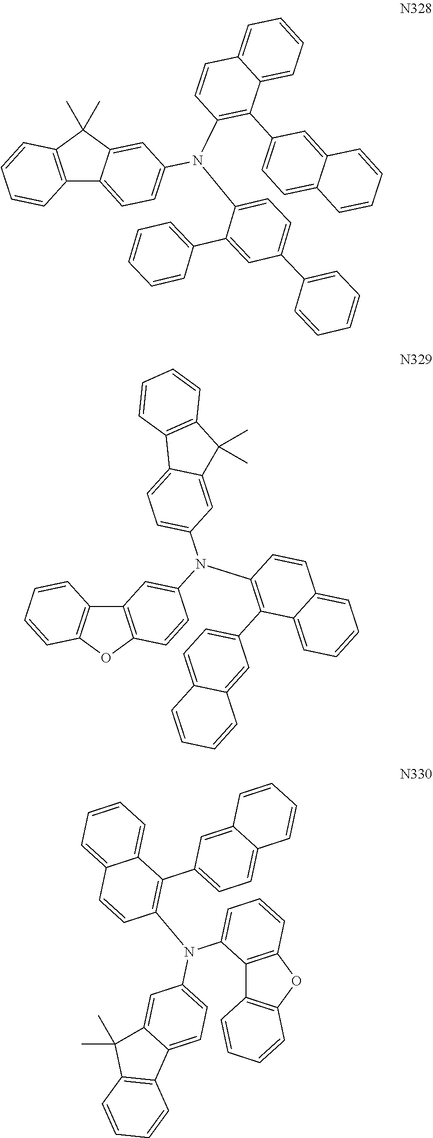 [SMILES: CC1(C)c2ccccc2-c2ccc(N(c3ccc(-c4ccccc4)cc3-c3ccccc3)c3ccc4ccccc4c3-c3ccc4ccccc4c3)cc21 ; CC1(C)c2ccccc2-c2ccc(N(c3ccc4oc5ccccc5c4c3)c3ccc4ccccc4c3-c3ccc4ccccc4c3)cc21 ; CC1(C)c2ccccc2-c2ccc(N(c3ccc4ccccc4c3-c3ccc4ccccc4c3)c3cccc4oc5ccccc5c34)cc21]

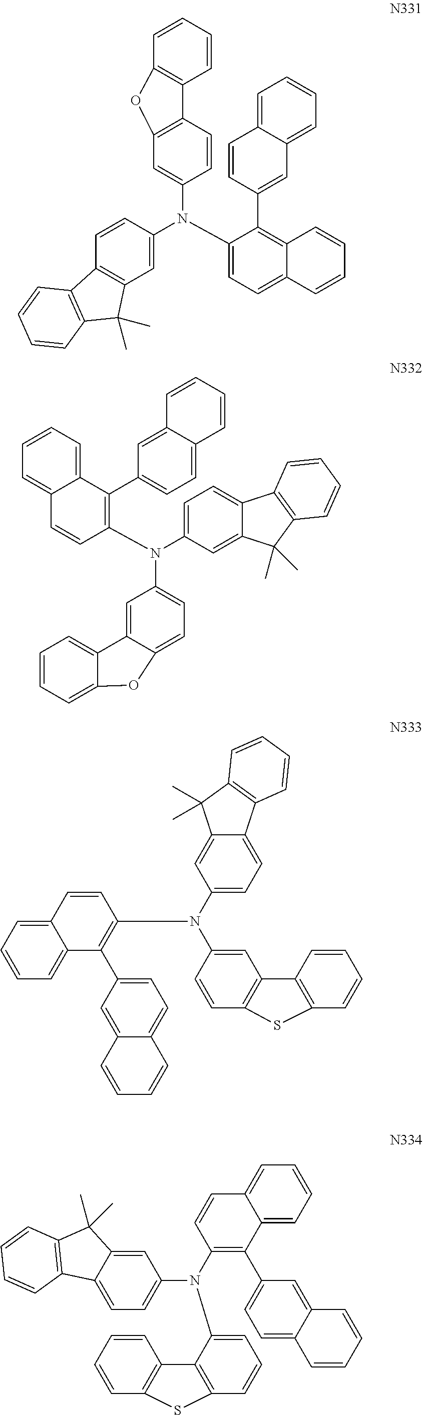 [SMILES: CC1(C)c2ccccc2-c2ccc(N(c3ccc4c(c3)oc3ccccc34)c3ccc4ccccc4c3-c3ccc4ccccc4c3)cc21 ; CC1(C)c2ccccc2-c2ccc(N(c3ccc4oc5ccccc5c4c3)c3ccc4ccccc4c3-c3ccc4ccccc4c3)cc21 ; CC1(C)c2ccccc2-c2ccc(N(c3ccc4sc5ccccc5c4c3)c3ccc4ccccc4c3-c3ccc4ccccc4c3)cc21 ; CC1(C)c2ccccc2-c2ccc(N(c3ccc4ccccc4c3-c3ccc4ccccc4c3)c3cccc4sc5ccccc5c34)cc21]

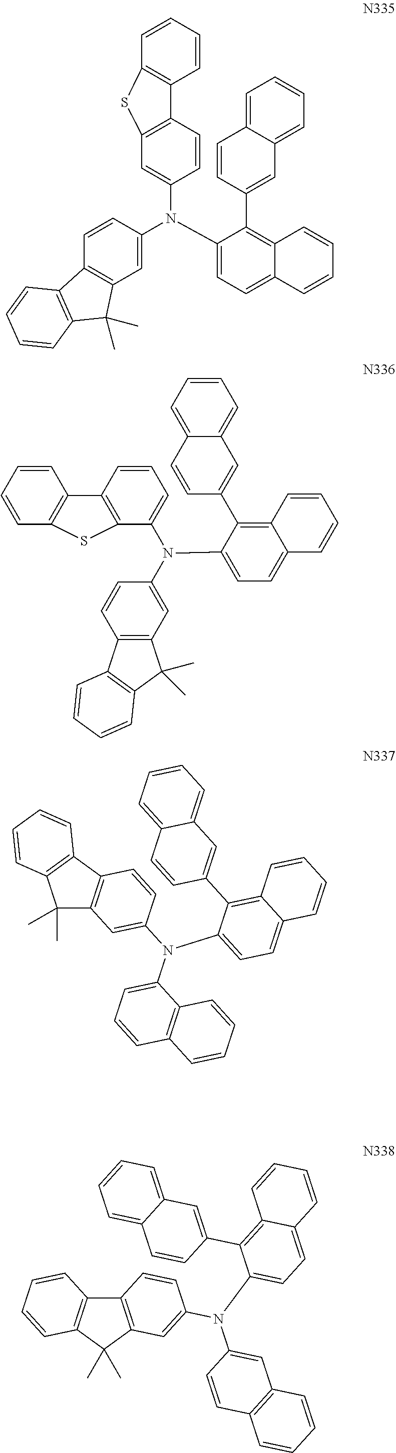 [SMILES: CC1(C)c2ccccc2-c2ccc(N(c3ccc4c(c3)sc3ccccc34)c3ccc4ccccc4c3-c3ccc4ccccc4c3)cc21 ; CC1(C)c2ccccc2-c2ccc(N(c3ccc4ccccc4c3-c3ccc4ccccc4c3)c3cccc4c3sc3ccccc34)cc21 ; CC1(C)c2ccccc2-c2ccc(N(c3ccc4ccccc4c3-c3ccc4ccccc4c3)c3cccc4ccccc34)cc21 ; CC1(C)c2ccccc2-c2ccc(N(c3ccc4ccccc4c3)c3ccc4ccccc4c3-c3ccc4ccccc4c3)cc21]

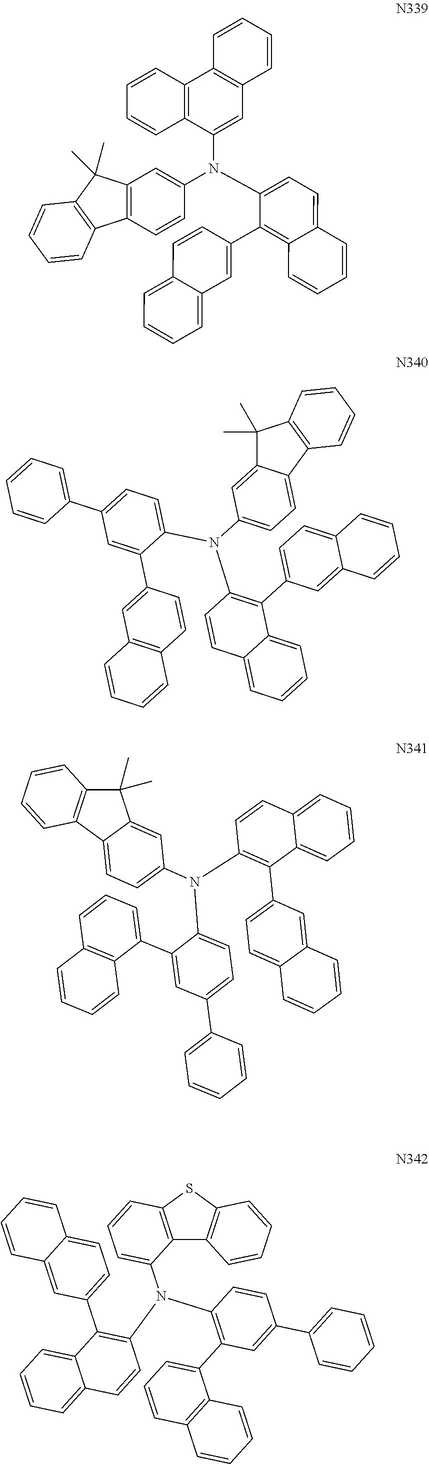 [SMILES: CC1(C)c2ccccc2-c2ccc(N(c3ccc4ccccc4c3-c3ccc4ccccc4c3)c3cc4ccccc4c4ccccc34)cc21 ; CC1(C)c2ccccc2-c2ccc(N(c3ccc(-c4ccccc4)cc3-c3ccc4ccccc4c3)c3ccc4ccccc4c3-c3ccc4ccccc4c3)cc21 ; CC1(C)c2ccccc2-c2ccc(N(c3ccc(-c4ccccc4)cc3-c3cccc4ccccc34)c3ccc4ccccc4c3-c3ccc4ccccc4c3)cc21 ; c1ccc(-c2ccc(N(c3ccc4ccccc4c3-c3ccc4ccccc4c3)c3cccc4sc5ccccc5c34)c(-c3cccc4ccccc34)c2)cc1]

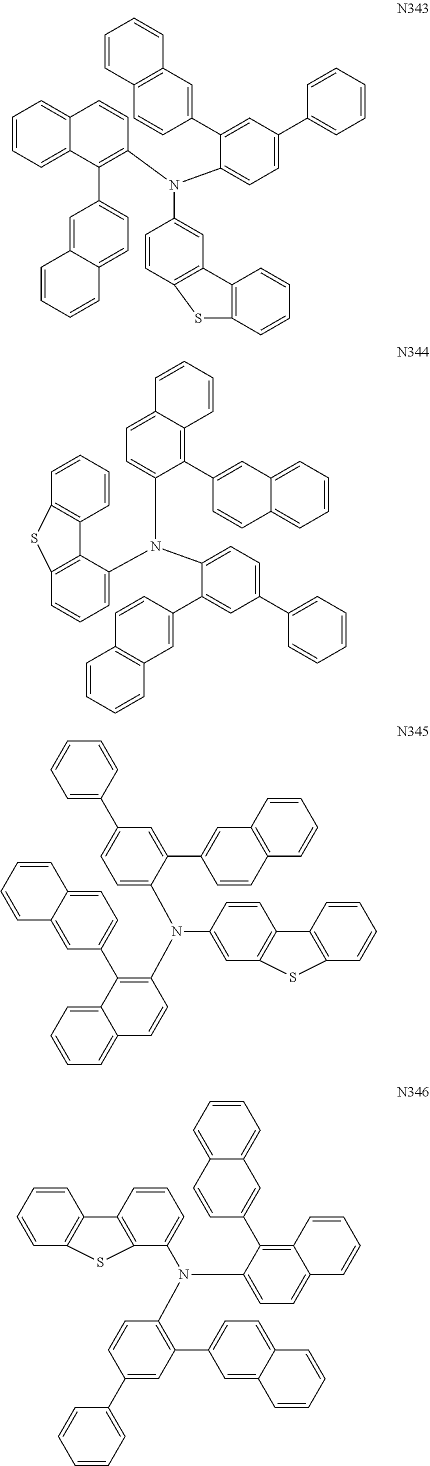

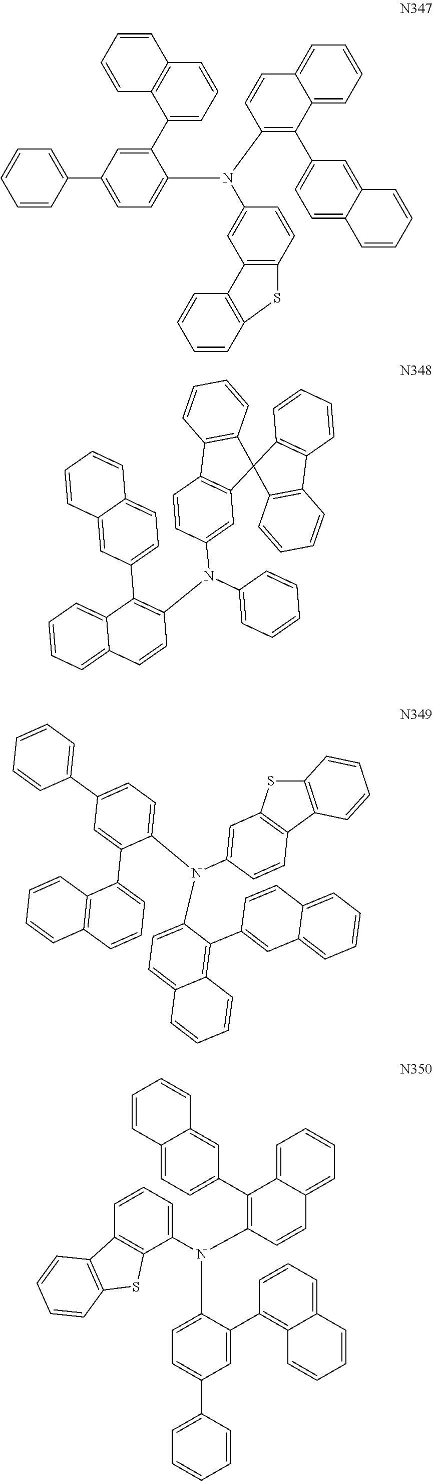

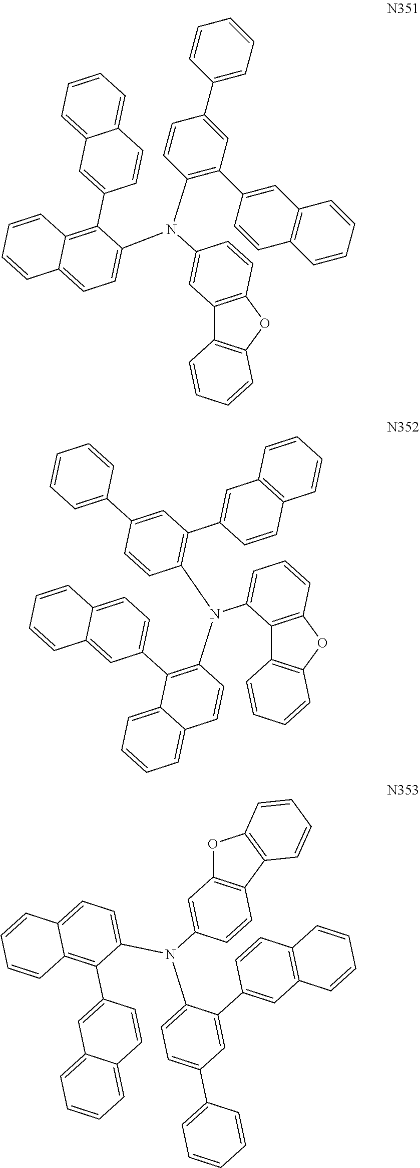 [SMILES: c1ccc(-c2ccc(N(c3ccc4oc5ccccc5c4c3)c3ccc4ccccc4c3-c3ccc4ccccc4c3)c(-c3ccc4ccccc4c3)c2)cc1 ; c1ccc(-c2ccc(N(c3ccc4ccccc4c3-c3ccc4ccccc4c3)c3cccc4oc5ccccc5c34)c(-c3ccc4ccccc4c3)c2)cc1 ; c1ccc(-c2ccc(N(c3ccc4c(c3)oc3ccccc34)c3ccc4ccccc4c3-c3ccc4ccccc4c3)c(-c3ccc4ccccc4c3)c2)cc1]

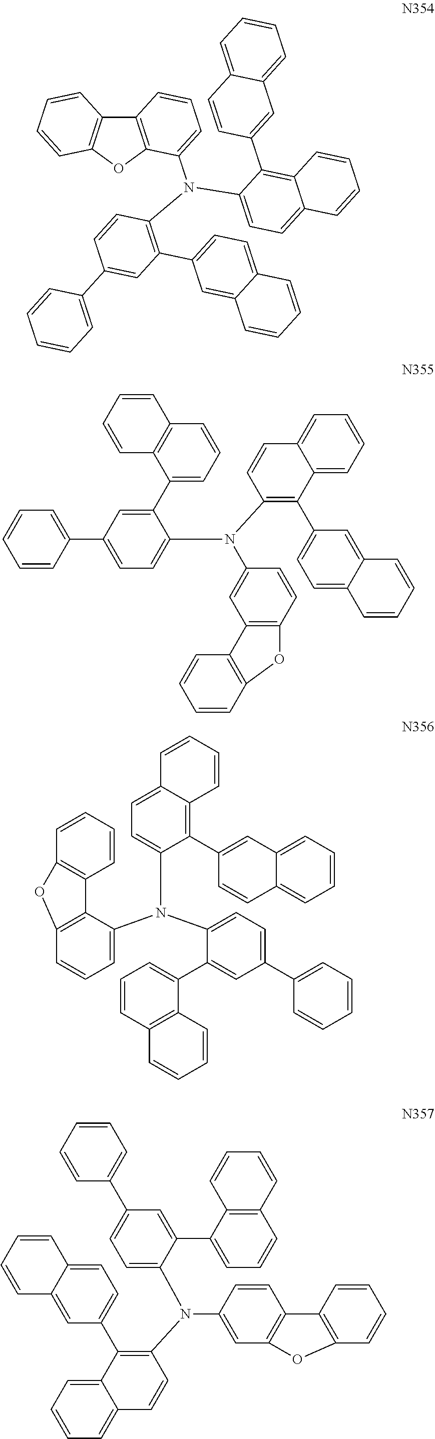

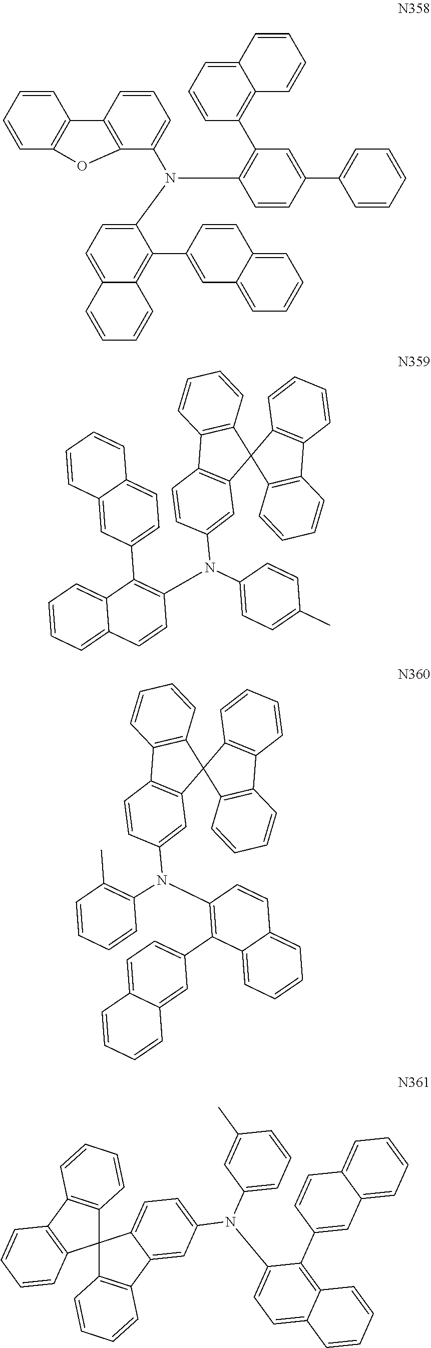

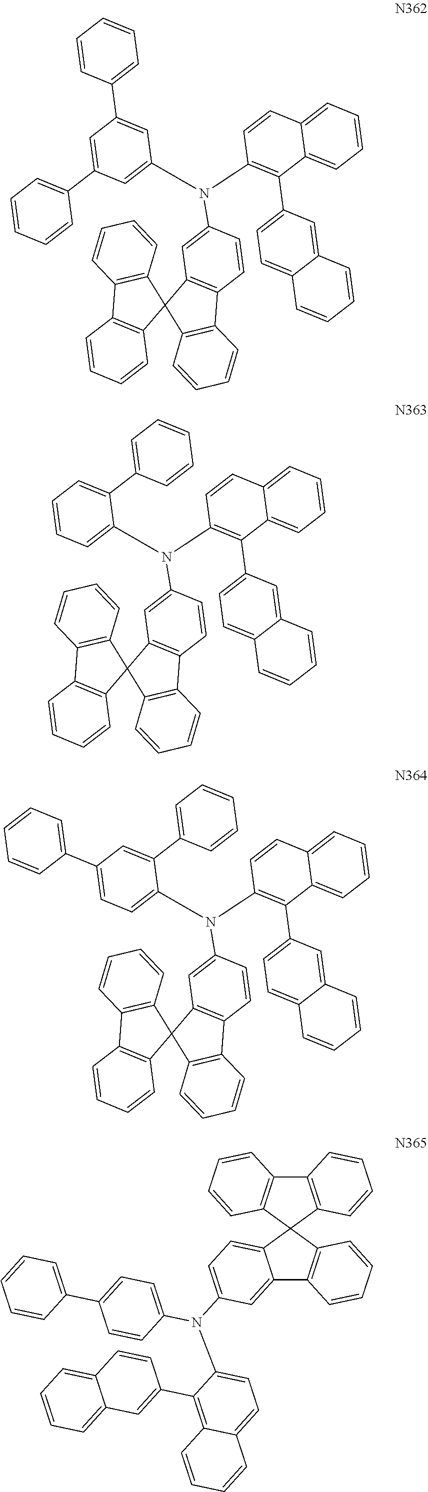

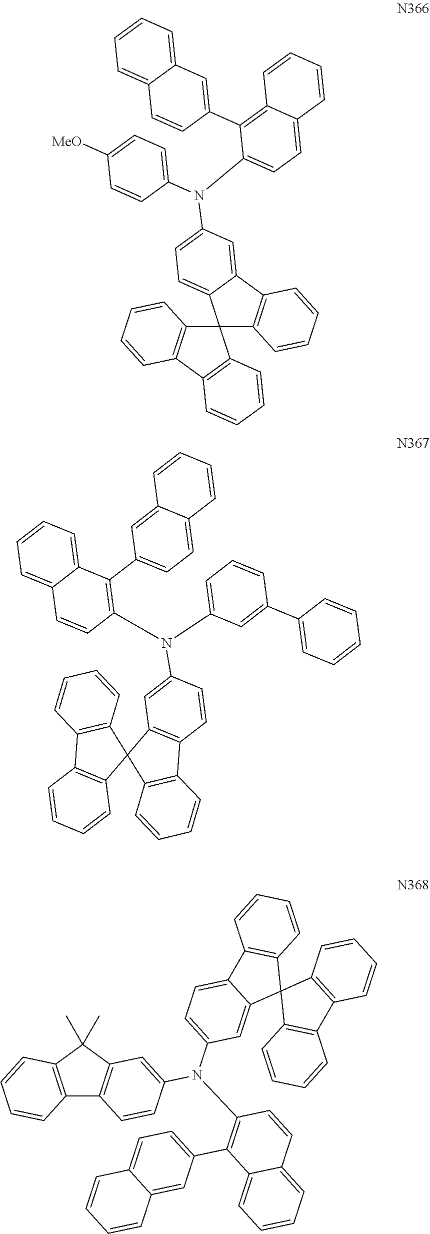 [SMILES: COc1ccc(N(c2ccc3c(c2)-c2ccccc2C32c3ccccc3-c3ccccc32)c2ccc3ccccc3c2-c2ccc3ccccc3c2)cc1 ; c1ccc(-c2cccc(N(c3ccc4c(c3)C3(c5ccccc5-c5ccccc53)c3ccccc3-4)c3ccc4ccccc4c3-c3ccc4ccccc4c3)c2)cc1 ; CC1(C)c2ccccc2-c2ccc(N(c3ccc4c(c3)C3(c5ccccc5-c5ccccc53)c3ccccc3-4)c3ccc4ccccc4c3-c3ccc4ccccc4c3)cc21]

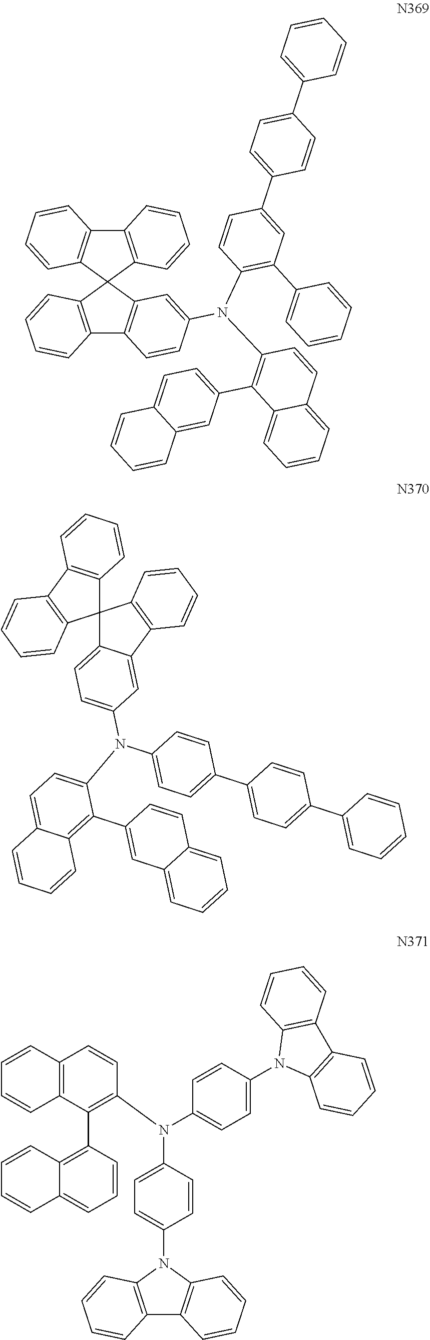 [SMILES: c1ccc(-c2ccc(-c3ccc(N(c4ccc5c(c4)C4(c6ccccc6-c6ccccc64)c4ccccc4-5)c4ccc5ccccc5c4-c4ccc5ccccc5c4)c(-c4ccccc4)c3)cc2)cc1 ; c1ccc(-c2ccc(-c3ccc(N(c4ccc5c(c4)-c4ccccc4C54c5ccccc5-c5ccccc54)c4ccc5ccccc5c4-c4ccc5ccccc5c4)cc3)cc2)cc1 ; c1ccc2c(-c3c(N(c4ccc(-n5c6ccccc6c6ccccc65)cc4)c4ccc(-n5c6ccccc6c6ccccc65)cc4)ccc4ccccc34)cccc2c1]

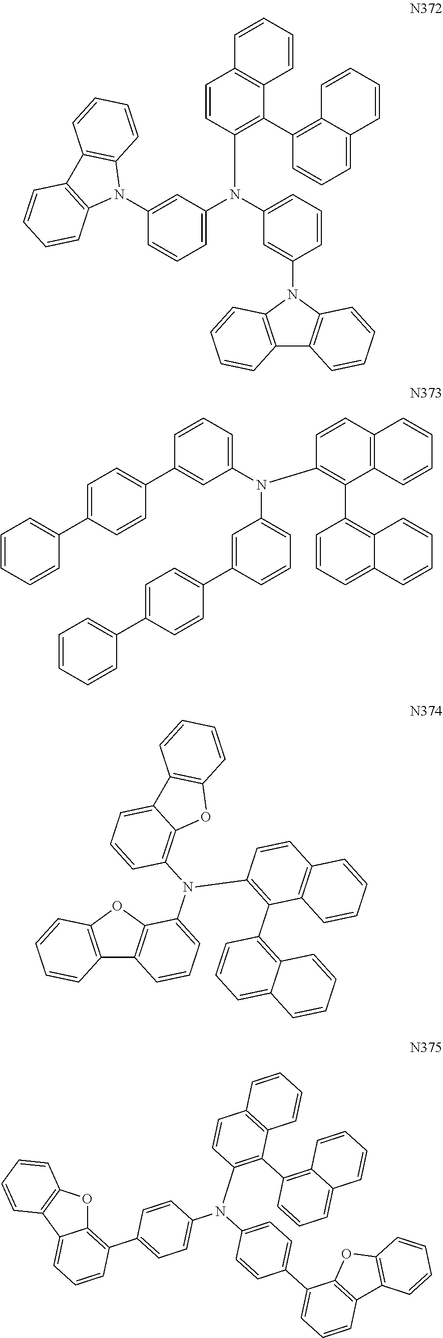 [SMILES: c1cc(N(c2cccc(-n3c4ccccc4c4ccccc43)c2)c2ccc3ccccc3c2-c2cccc3ccccc23)cc(-n2c3ccccc3c3ccccc32)c1 ; c1ccc(-c2ccc(-c3cccc(N(c4cccc(-c5ccc(-c6ccccc6)cc5)c4)c4ccc5ccccc5c4-c4cccc5ccccc45)c3)cc2)cc1 ; c1ccc2c(-c3c(N(c4cccc5c4oc4ccccc45)c4cccc5c4oc4ccccc45)ccc4ccccc34)cccc2c1 ; c1ccc2c(-c3c(N(c4ccc(-c5cccc6c5oc5ccccc56)cc4)c4ccc(-c5cccc6c5oc5ccccc56)cc4)ccc4ccccc34)cccc2c1]

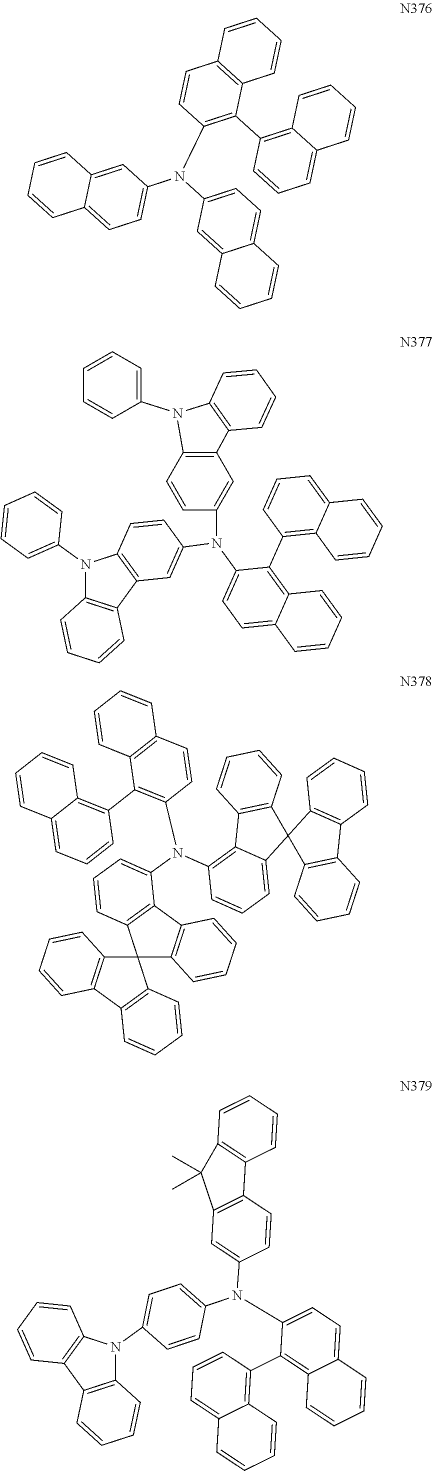

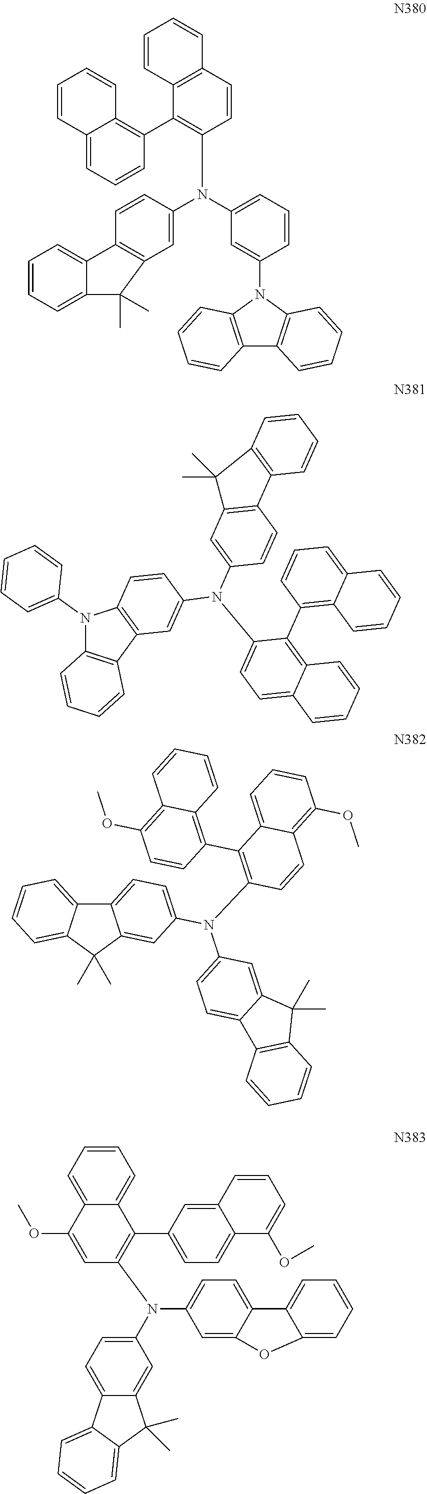

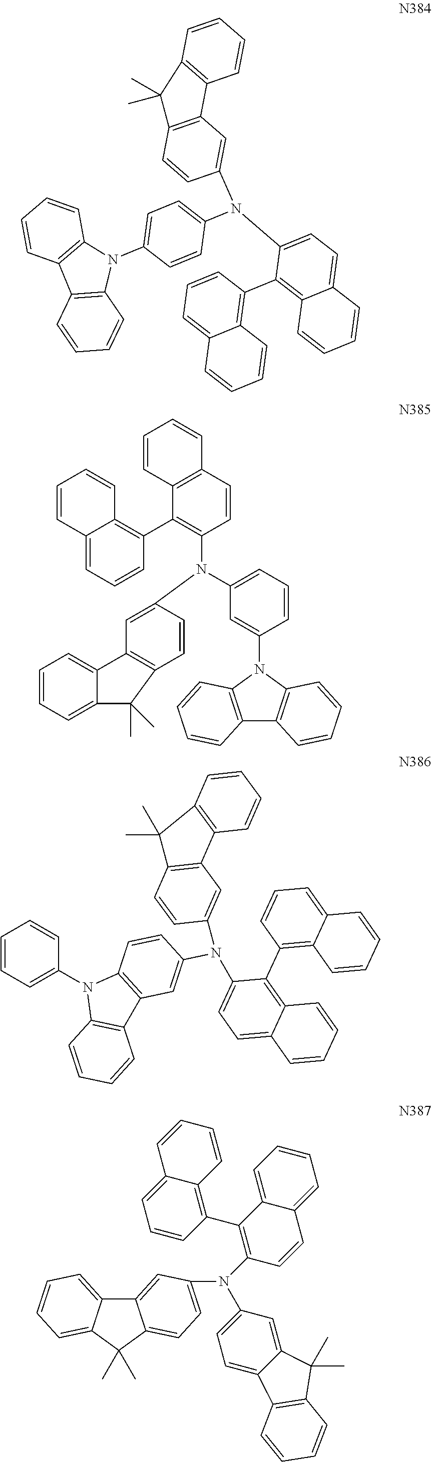

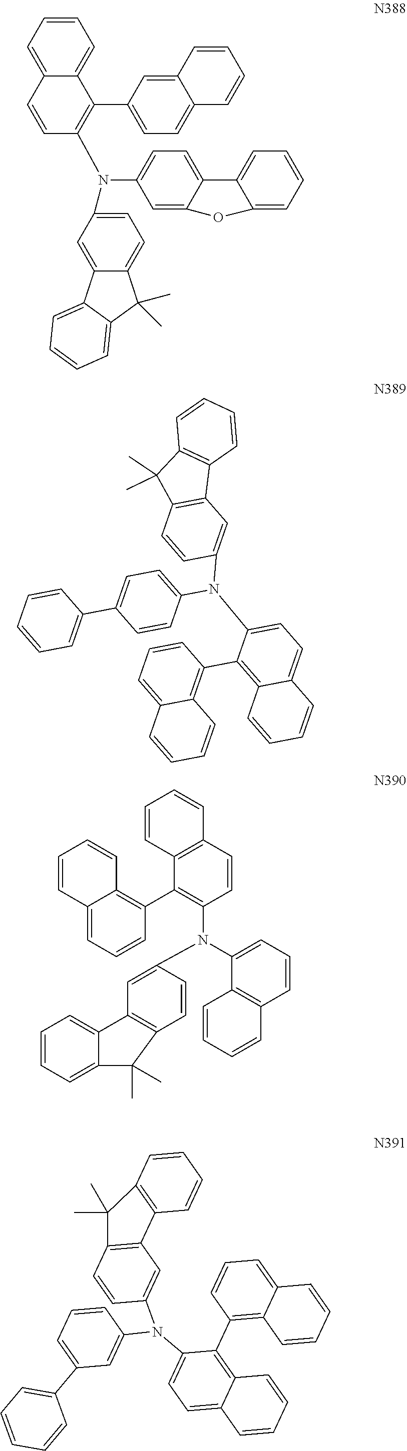

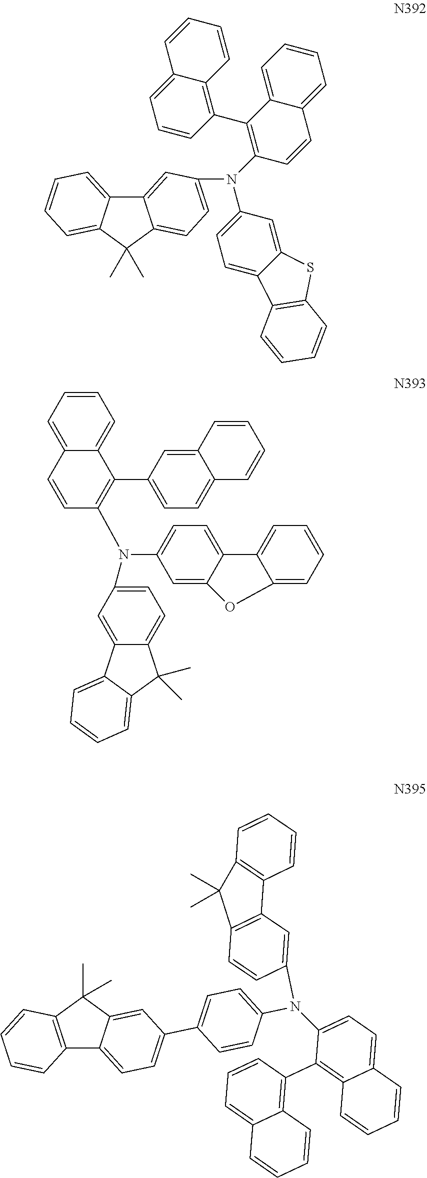 [SMILES: CC1(C)c2ccccc2-c2cc(N(c3ccc4c(c3)sc3ccccc34)c3ccc4ccccc4c3-c3cccc4ccccc34)ccc21 ; CC1(C)c2ccccc2-c2cc(N(c3ccc4c(c3)oc3ccccc34)c3ccc4ccccc4c3-c3ccc4ccccc4c3)ccc21 ; CC1(C)c2ccccc2-c2cc(N(c3ccc(-c4ccc5c(c4)C(C)(C)c4ccccc4-5)cc3)c3ccc4ccccc4c3-c3cccc4ccccc34)ccc21]

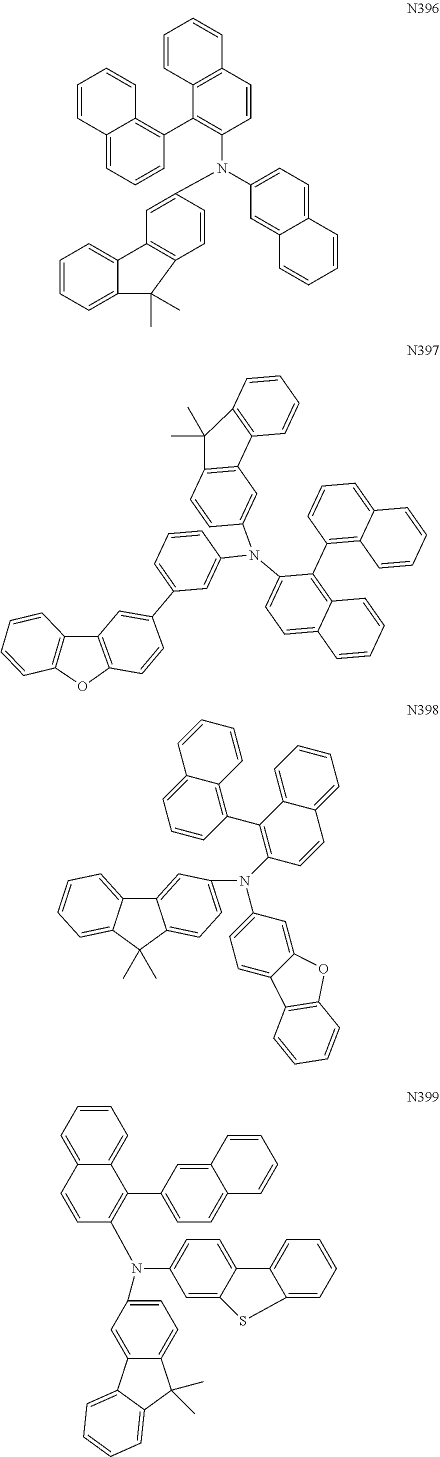 [SMILES: CC1(C)c2ccccc2-c2cc(N(c3ccc4ccccc4c3)c3ccc4ccccc4c3-c3cccc4ccccc34)ccc21 ; CC1(C)c2ccccc2-c2cc(N(c3cccc(-c4ccc5oc6ccccc6c5c4)c3)c3ccc4ccccc4c3-c3cccc4ccccc34)ccc21 ; CC1(C)c2ccccc2-c2cc(N(c3ccc4c(c3)oc3ccccc34)c3ccc4ccccc4c3-c3cccc4ccccc34)ccc21 ; CC1(C)c2ccccc2-c2cc(N(c3ccc4c(c3)sc3ccccc34)c3ccc4ccccc4c3-c3ccc4ccccc4c3)ccc21]

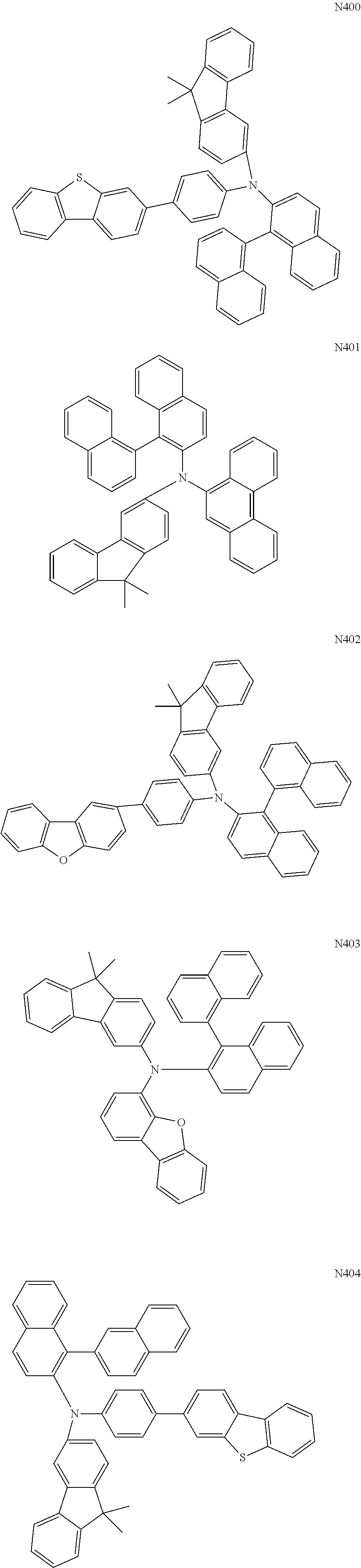 [SMILES: CC1(C)c2ccccc2-c2cc(N(c3ccc(-c4ccc5c(c4)sc4ccccc45)cc3)c3ccc4ccccc4c3-c3cccc4ccccc34)ccc21 ; CC1(C)c2ccccc2-c2cc(N(c3ccc4ccccc4c3-c3cccc4ccccc34)c3cc4ccccc4c4ccccc34)ccc21 ; CC1(C)c2ccccc2-c2cc(N(c3ccc(-c4ccc5oc6ccccc6c5c4)cc3)c3ccc4ccccc4c3-c3cccc4ccccc34)ccc21 ; CC1(C)c2ccccc2-c2cc(N(c3ccc4ccccc4c3-c3cccc4ccccc34)c3cccc4c3oc3ccccc34)ccc21 ; CC1(C)c2ccccc2-c2cc(N(c3ccc(-c4ccc5c(c4)sc4ccccc45)cc3)c3ccc4ccccc4c3-c3ccc4ccccc4c3)ccc21]

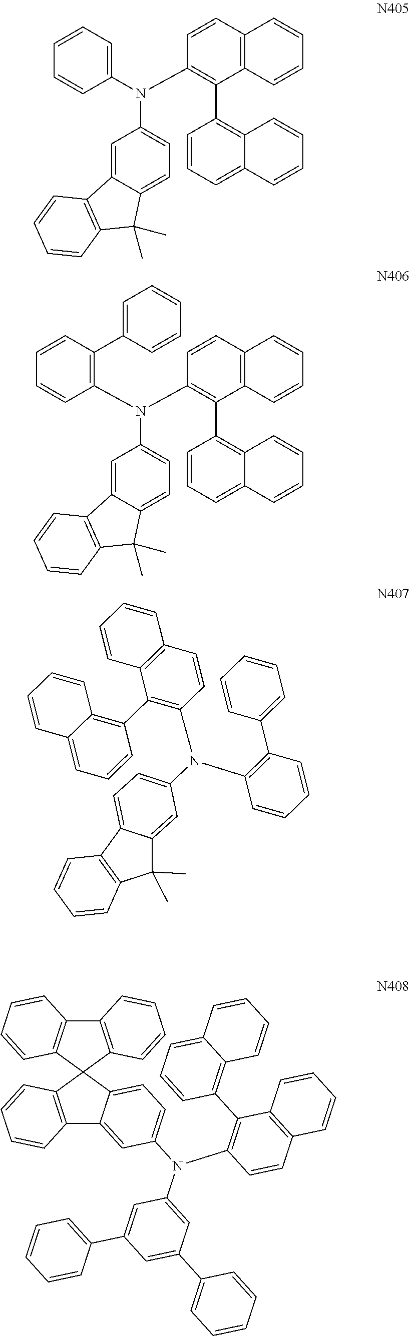

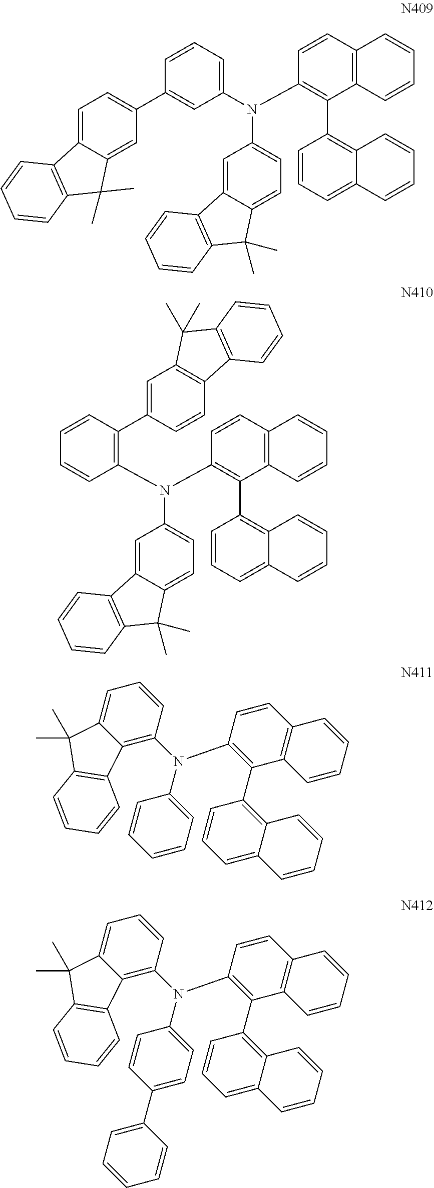

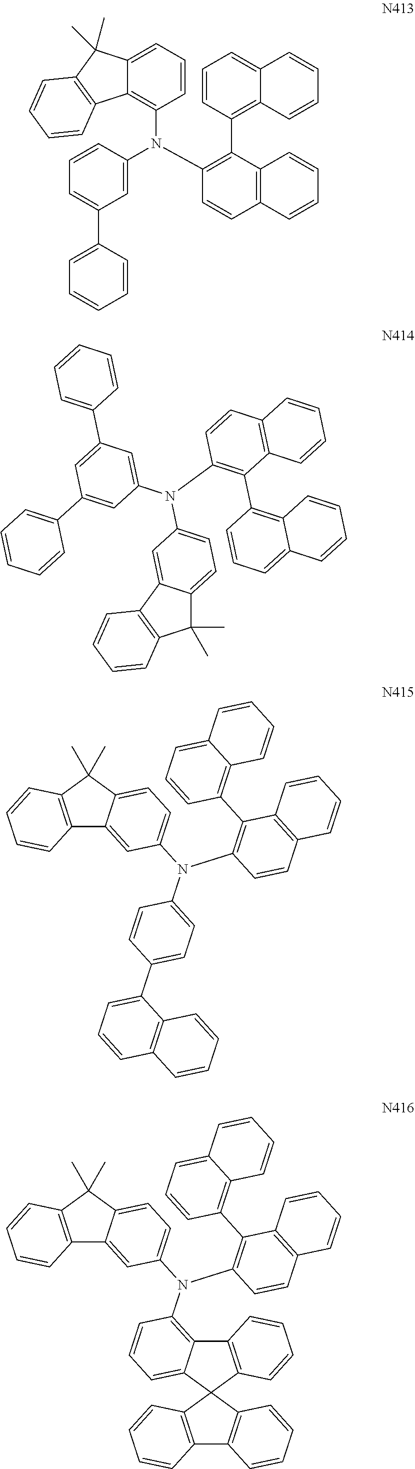

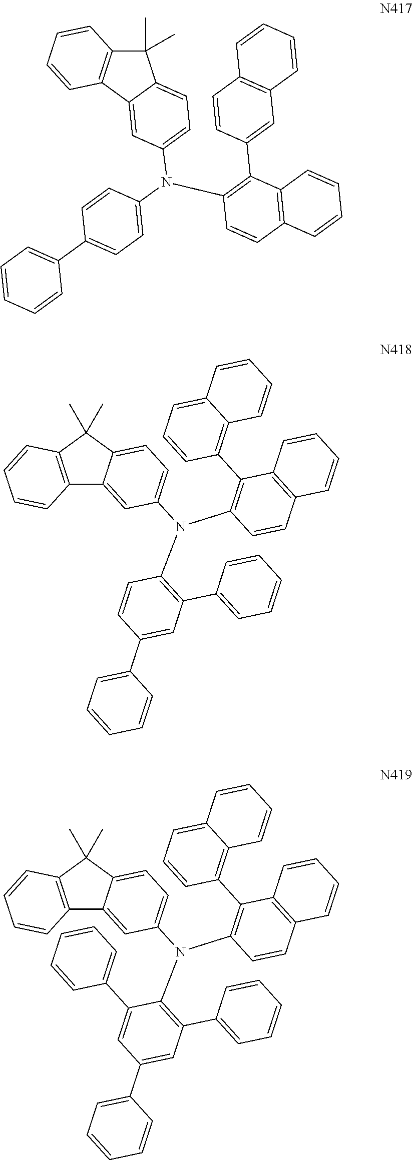 [SMILES: CC1(C)c2ccccc2-c2cc(N(c3ccc(-c4ccccc4)cc3)c3ccc4ccccc4c3-c3ccc4ccccc4c3)ccc21 ; CC1(C)c2ccccc2-c2cc(N(c3ccc(-c4ccccc4)cc3-c3ccccc3)c3ccc4ccccc4c3-c3cccc4ccccc34)ccc21 ; CC1(C)c2ccccc2-c2cc(N(c3ccc4ccccc4c3-c3cccc4ccccc34)c3c(-c4ccccc4)cc(-c4ccccc4)cc3-c3ccccc3)ccc21]

8. The compound according to claim 1, wherein the compound has a structure as shown in Formula (III): 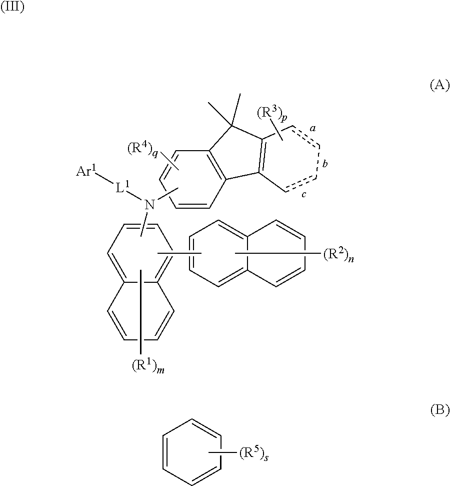
wherein Formula (B) is fused to Formula (A) along any one of the dotted line of a, b or c;
L¹ is selected from one of a single bond, a substituted or non-substituted C₁-C₁₀ alkylene, a substituted or non-substituted C₆-C₃₀ arylene, a substituted or non-substituted C₃-C₃₀ heteroarylene group;
Ar¹ is selected from one of a substituted or unsubstituted C₆-C₃₀ aryl, a substituted or unsubstituted C₃-C₃₀ heteroaryl;
R¹, R², R³, R⁴ and R⁵ are each independently selected from halogen, amino, cyano, nitryl, an ester group, hydroxyl, a C₁-C₁₀ silicyl, a substituted or unsubstituted C₁-C₁₀ chain-typed alkyl, a substituted or unsubstituted C₃-C₁₀ cycloalkyl, a substituted or unsubstituted C₂-C₁₀ alkenyl, a substituted or unsubstituted C₂-C₁₀ alkynyl, a substituted or unsubstituted C₁-C₁₀ chain-typed alkoxy, a substituted or unsubstituted C₃-C₁₀ cycloalkoxy, a substituted or unsubstituted C₆-C₃₀ arylamino, a substituted or unsubstituted C₃-C₃₀ heteroarylamino, a substituted or unsubstituted C₆-C₃₀ aryl, a substituted or unsubstituted C₃-C₃₀ heteroaryl;
m is an integer of 0-6, and when m≥2, R¹ is same or different;
n is an integer of 0-7, and when n≥2, R² is same or different;
p is an integer of 0-2, and when p=2, R³ is same or different;
q is an integer of 0-3, and when q≥2, R⁴ is same or different;
s is an integer of 0-4, and when s≥2, R⁵ is same or different;
when the above-mentioned groups have a substituent, the substituent is selected from one or a combination of at least two of halogen, cyano, a C₁-C₁₀ chain-typed alkyl, a C₃-C₁₀ cycloalkyl, a C₁-C₆ alkoxy, a C₁-C₆ thioalkoxy, a C₆-C₃₀ arylamino, a C₃-C₃₀ heteroarylamino, a C₆-C₃₀ monocyclic aryl, a C₁₀-C₃₀ fused-cyclic aryl, a C₃-C₃₀ monocyclic heteroaryl, and a C₆-C₃₀ fused-cyclic heteroaryl.

9. The compound according to claim 8, wherein the compound has a structure as shown in the following Formula (3-1): 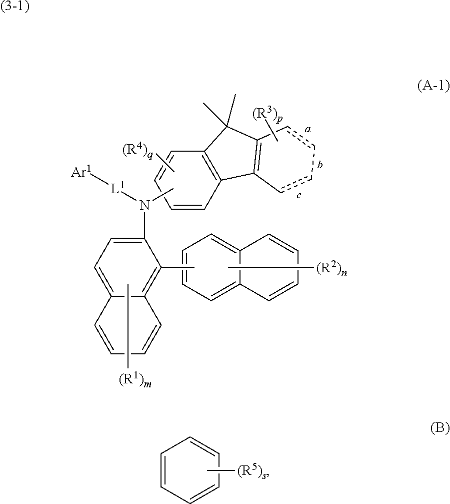
wherein Formula (B) is fused to Formula (A) along any one of the dotted line of a, b or c;
preferably, wherein the compound has a structure as shown in the following Formula (3-2): 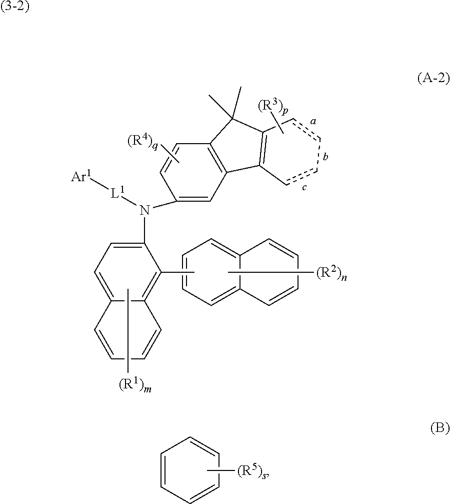
wherein Formula (B) is fused to Formula (A) along any one of the dotted line of a, b or c;
preferably, in Formula (3-2), wherein s, p, n, m and q are 0.

10. (canceled)

11. (canceled)

12. The compound according to claim 9, wherein fluorenyl and the benzene ring are fused in the b position;
preferably, wherein the L¹ is selected from a single bond, or a substituted or unsubstituted phenylene, preferably, a single bond;
the Ar¹ is selected from one of a substituted or unsubstituted phenyl, a substituted or unsubstituted naphthyl, a substituted or unsubstituted phenanthryl, a substituted or unsubstituted dibenzofuryl, a substituted or unsubstituted dibenzothienyl, and a substituted or unsubstituted carbazolyl;
the -L¹-Ar¹ is selected from one of phenyl, biphenyl, terphenyl, dibenzofuran, dibenzothiophene, carbazolyl or phenanthryl;
when the above-mentioned groups have a substituent, the substituent is selected from one or a combination of at least two of a halogen, cyano, a C₁-C₁₀ chain-typed alkyl, a C₃-C₁₀ cycloalkyl, a C₁-C₆ alkoxy, a C₁-C₆ thioalkoxy, a C₆-C₃₀ arylamino, a C₃-C₃₀ heteroarylamino, a C₆-C₃₀ monocyclic aryl, a C₁₀-C₃₀ fused-cyclic aryl, a C₃-C₃₀ monocyclic heteroaryl, and a C₆-C₃₀ fused-cyclic heteroaryl.

13. (canceled)

14. The compound according to claim 8, wherein the compound has a structure as shown in T1-T255:

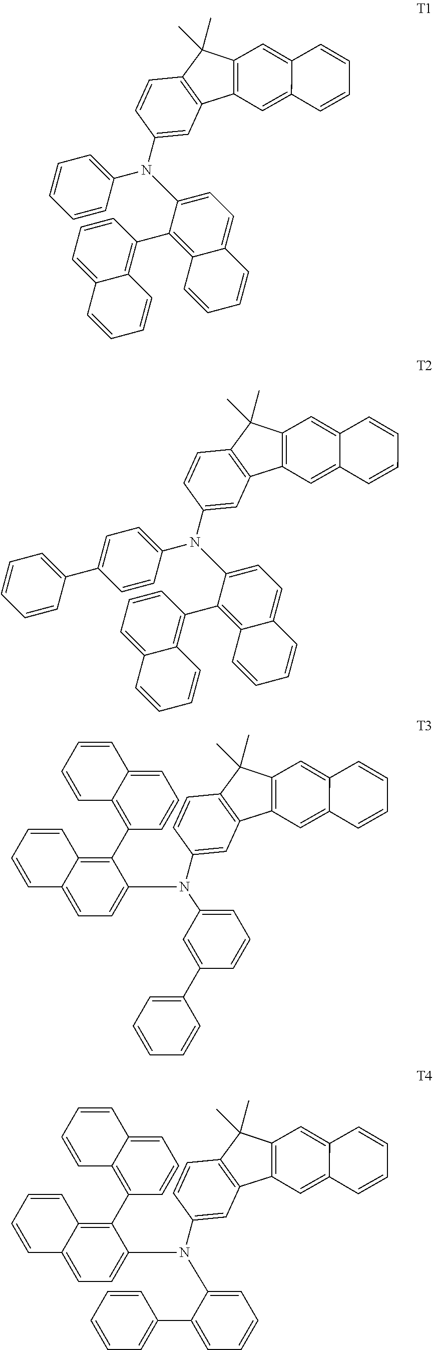 [SMILES: CC1(C)c2ccc(N(c3ccccc3)c3ccc4ccccc4c3-c3cccc4ccccc34)cc2-c2cc3ccccc3cc21 ; CC1(C)c2ccc(N(c3ccc(-c4ccccc4)cc3)c3ccc4ccccc4c3-c3cccc4ccccc34)cc2-c2cc3ccccc3cc21 ; CC1(C)c2ccc(N(c3cccc(-c4ccccc4)c3)c3ccc4ccccc4c3-c3cccc4ccccc34)cc2-c2cc3ccccc3cc21 ; CC1(C)c2ccc(N(c3ccccc3-c3ccccc3)c3ccc4ccccc4c3-c3cccc4ccccc34)cc2-c2cc3ccccc3cc21]

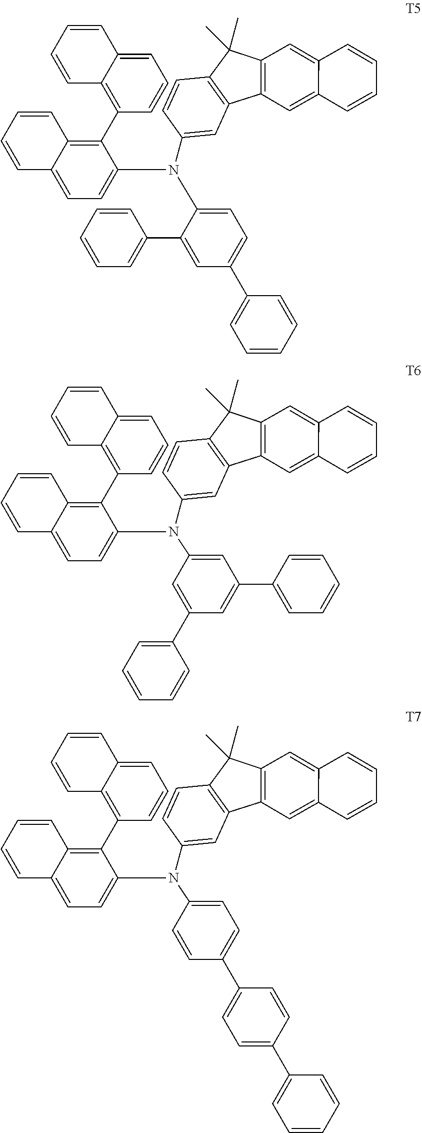

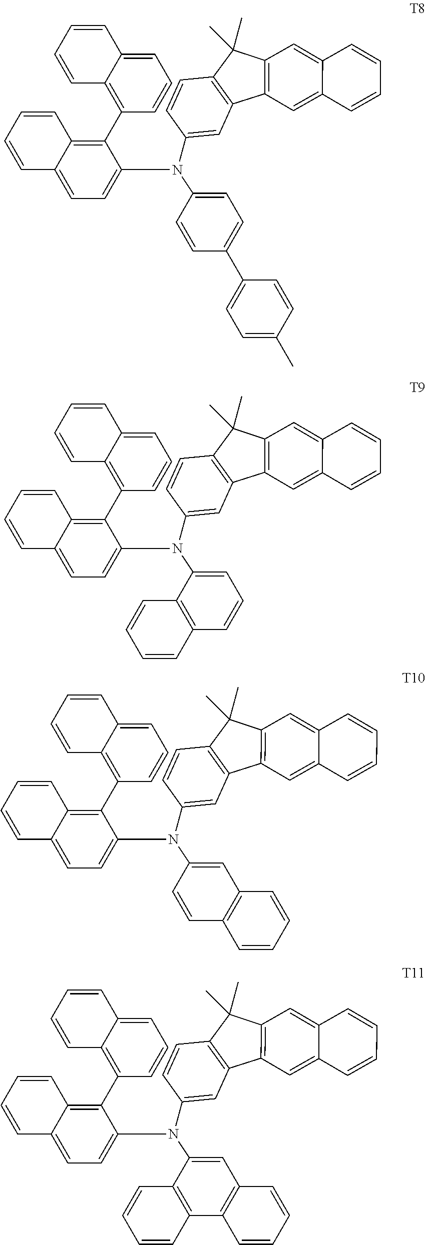

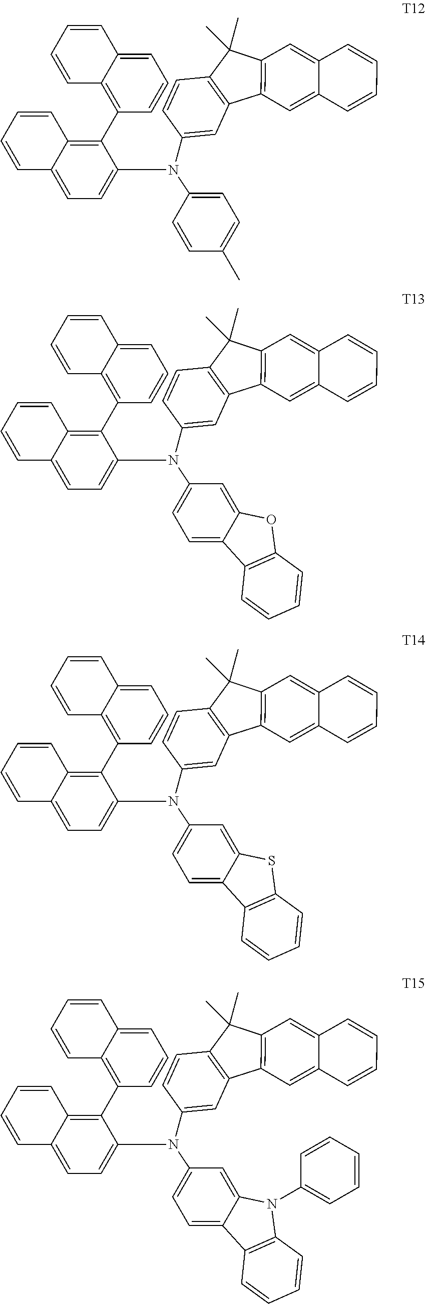

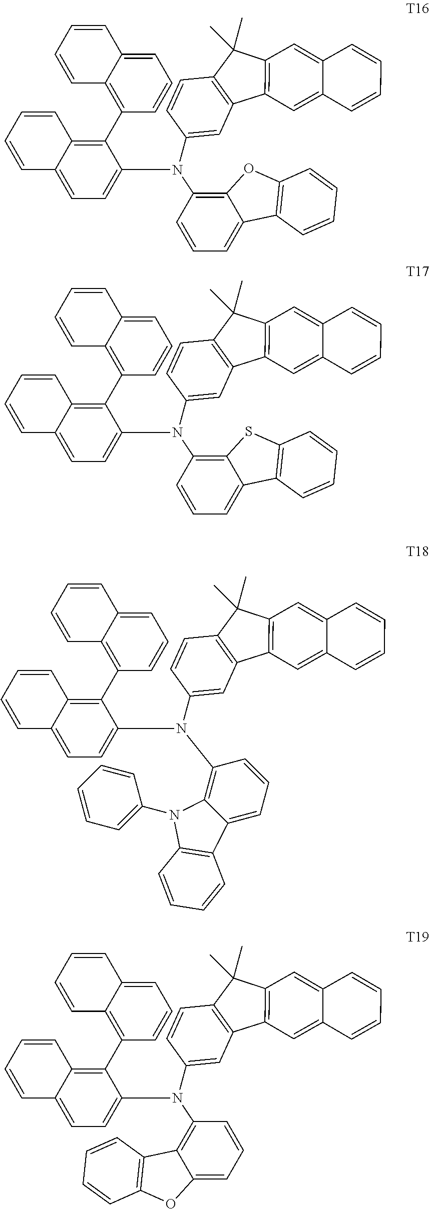

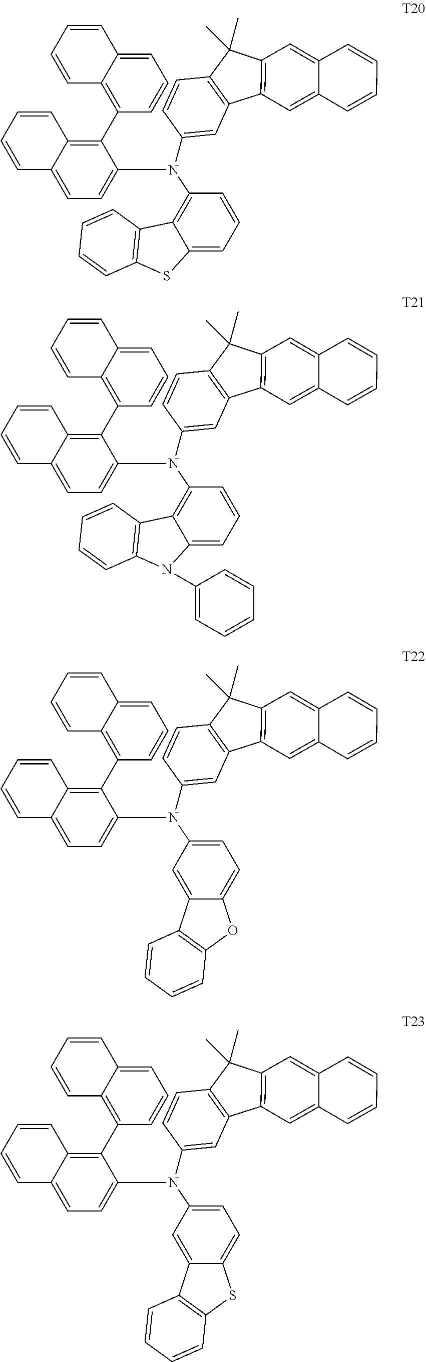

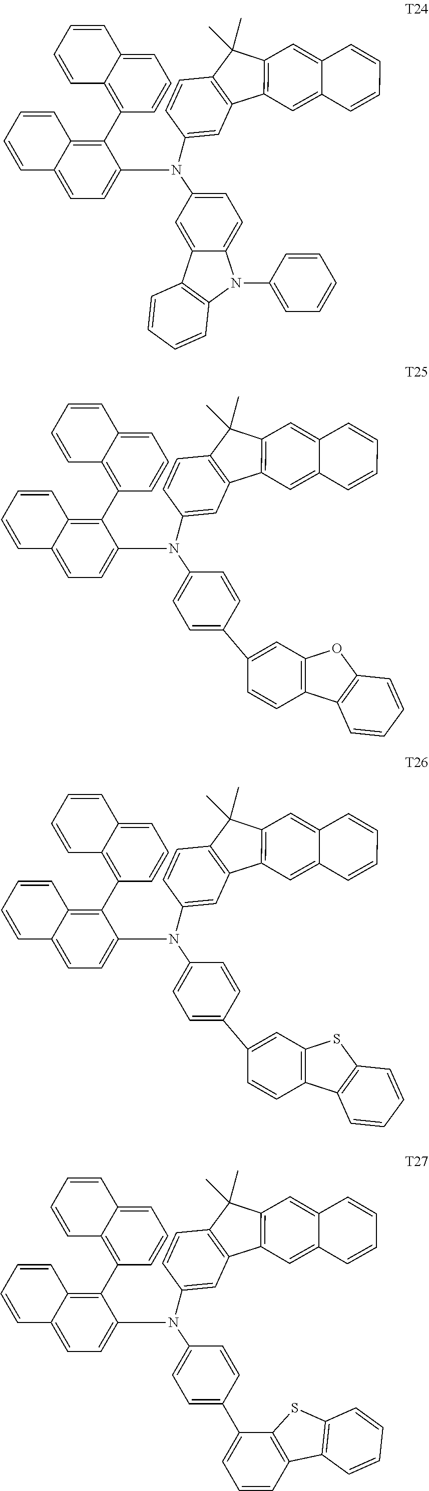 [SMILES: CC1(C)c2ccc(N(c3ccc4c(c3)c3ccccc3n4-c3ccccc3)c3ccc4ccccc4c3-c3cccc4ccccc34)cc2-c2cc3ccccc3cc21 ; CC1(C)c2ccc(N(c3ccc(-c4ccc5c(c4)oc4ccccc45)cc3)c3ccc4ccccc4c3-c3cccc4ccccc34)cc2-c2cc3ccccc3cc21 ; CC1(C)c2ccc(N(c3ccc(-c4ccc5c(c4)sc4ccccc45)cc3)c3ccc4ccccc4c3-c3cccc4ccccc34)cc2-c2cc3ccccc3cc21 ; CC1(C)c2ccc(N(c3ccc(-c4cccc5c4sc4ccccc45)cc3)c3ccc4ccccc4c3-c3cccc4ccccc34)cc2-c2cc3ccccc3cc21]

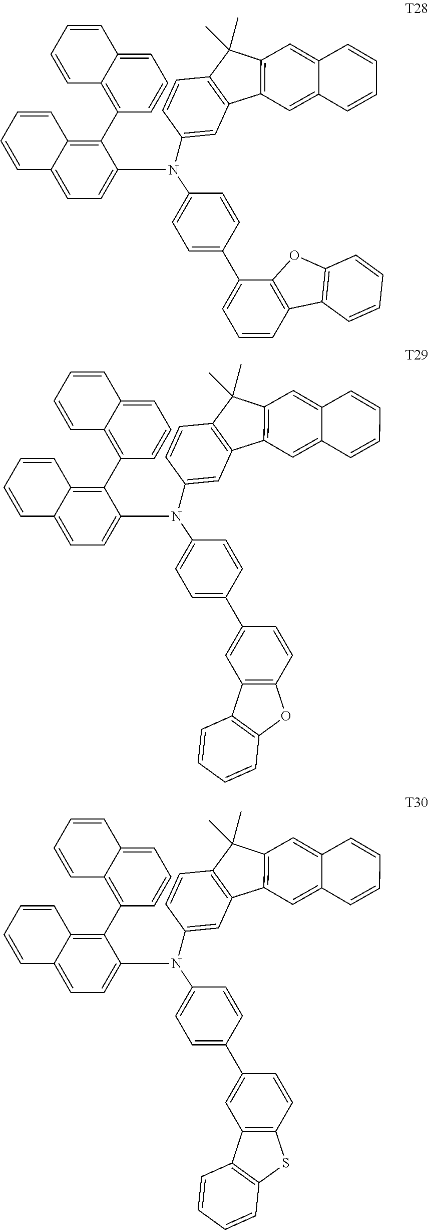

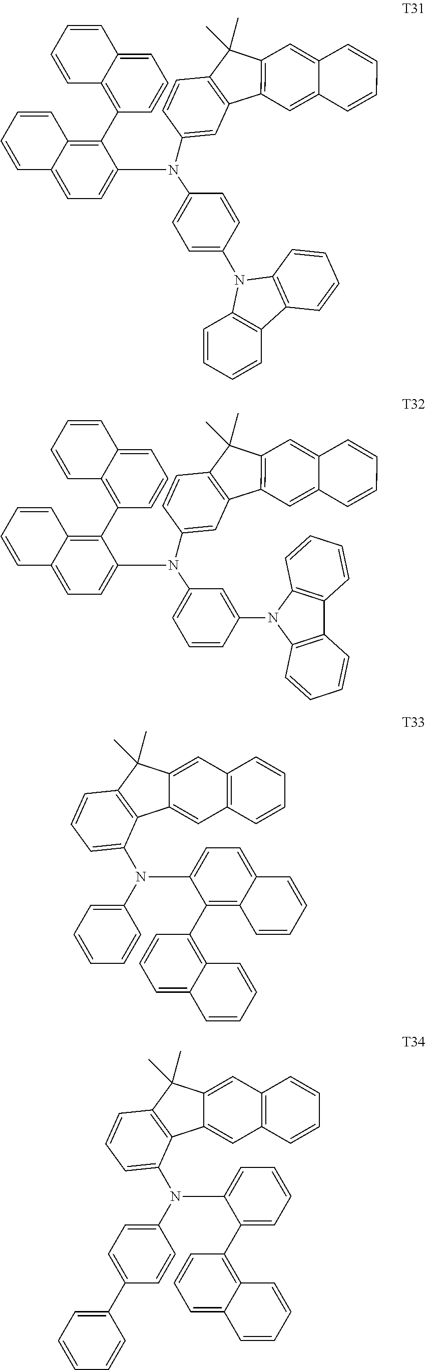

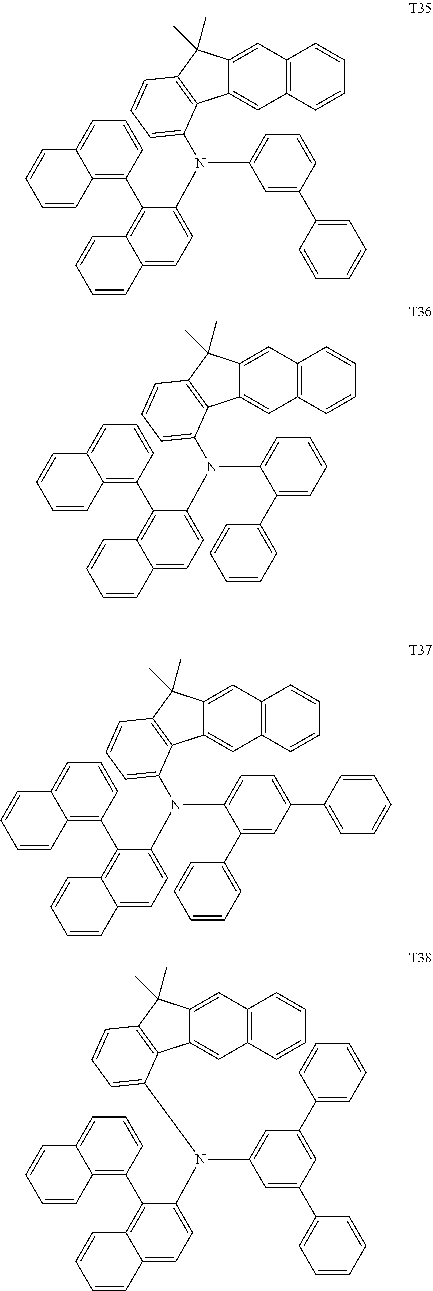 [SMILES: CC1(C)c2cc3ccccc3cc2-c2c(N(c3cccc(-c4ccccc4)c3)c3ccc4ccccc4c3-c3cccc4ccccc34)cccc21 ; CC1(C)c2cc3ccccc3cc2-c2c(N(c3ccccc3-c3ccccc3)c3ccc4ccccc4c3-c3cccc4ccccc34)cccc21 ; CC1(C)c2cc3ccccc3cc2-c2c(N(c3ccc(-c4ccccc4)cc3-c3ccccc3)c3ccc4ccccc4c3-c3cccc4ccccc34)cccc21 ; CC1(C)c2cc3ccccc3cc2-c2c(N(c3cc(-c4ccccc4)cc(-c4ccccc4)c3)c3ccc4ccccc4c3-c3cccc4ccccc34)cccc21]

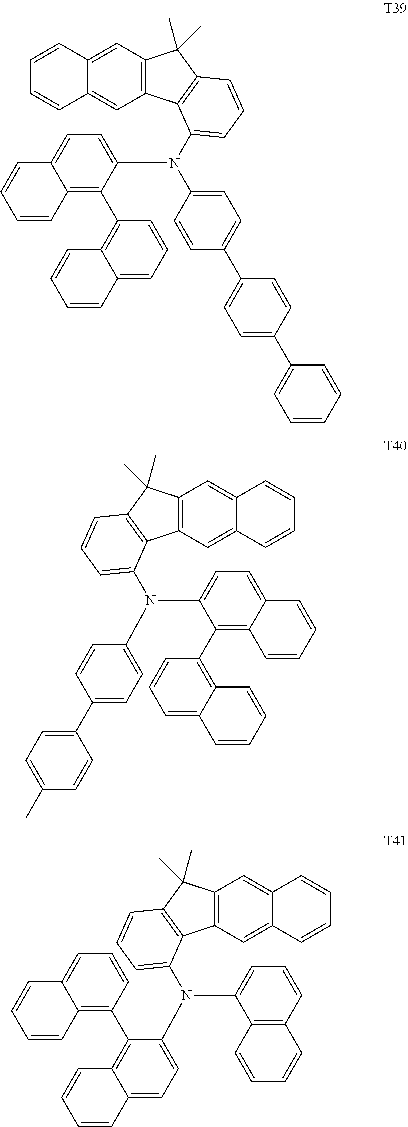 [SMILES: CC1(C)c2cc3ccccc3cc2-c2c(N(c3ccc(-c4ccc(-c5ccccc5)cc4)cc3)c3ccc4ccccc4c3-c3cccc4ccccc34)cccc21 ; Cc1ccc(-c2ccc(N(c3cccc4c3-c3cc5ccccc5cc3C4(C)C)c3ccc4ccccc4c3-c3cccc4ccccc34)cc2)cc1 ; CC1(C)c2cc3ccccc3cc2-c2c(N(c3ccc4ccccc4c3-c3cccc4ccccc34)c3cccc4ccccc34)cccc21]

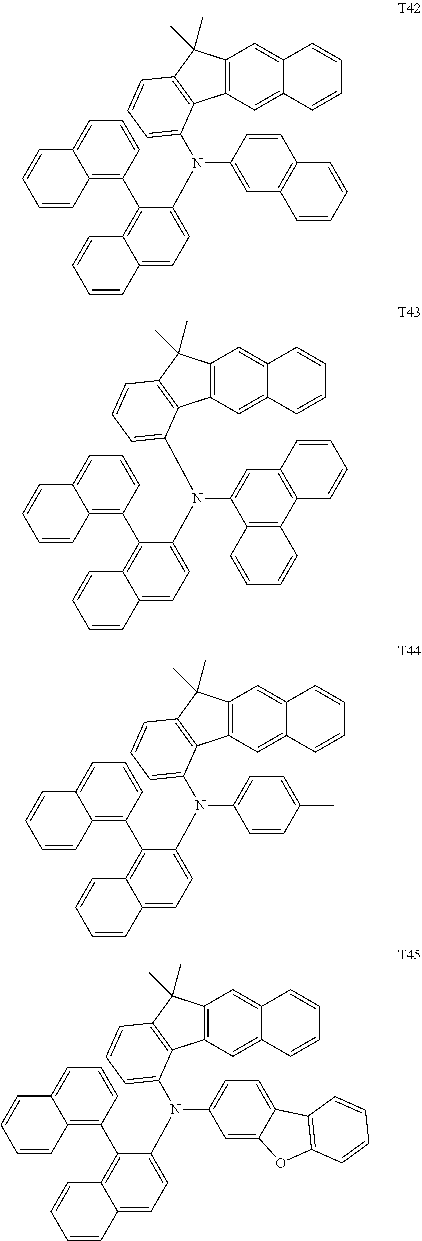

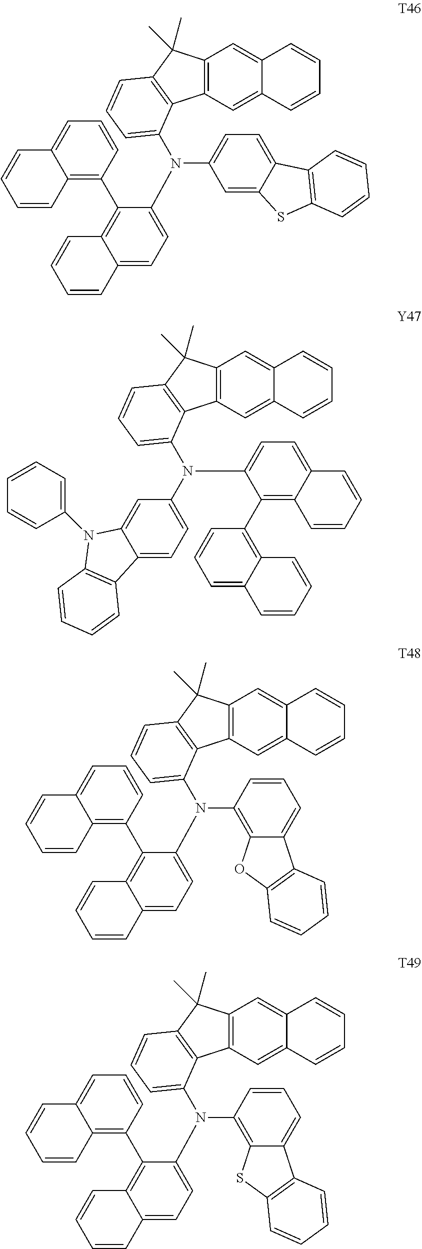 [SMILES: CC1(C)c2cc3ccccc3cc2-c2c(N(c3ccc4c(c3)sc3ccccc34)c3ccc4ccccc4c3-c3cccc4ccccc34)cccc21 ; CC1(C)c2cc3ccccc3cc2-c2c(N(c3ccc4c5ccccc5n(-c5ccccc5)c4c3)c3ccc4ccccc4c3-c3cccc4ccccc34)cccc21 ; CC1(C)c2cc3ccccc3cc2-c2c(N(c3ccc4ccccc4c3-c3cccc4ccccc34)c3cccc4c3oc3ccccc34)cccc21 ; CC1(C)c2cc3ccccc3cc2-c2c(N(c3ccc4ccccc4c3-c3cccc4ccccc34)c3cccc4c3sc3ccccc34)cccc21]

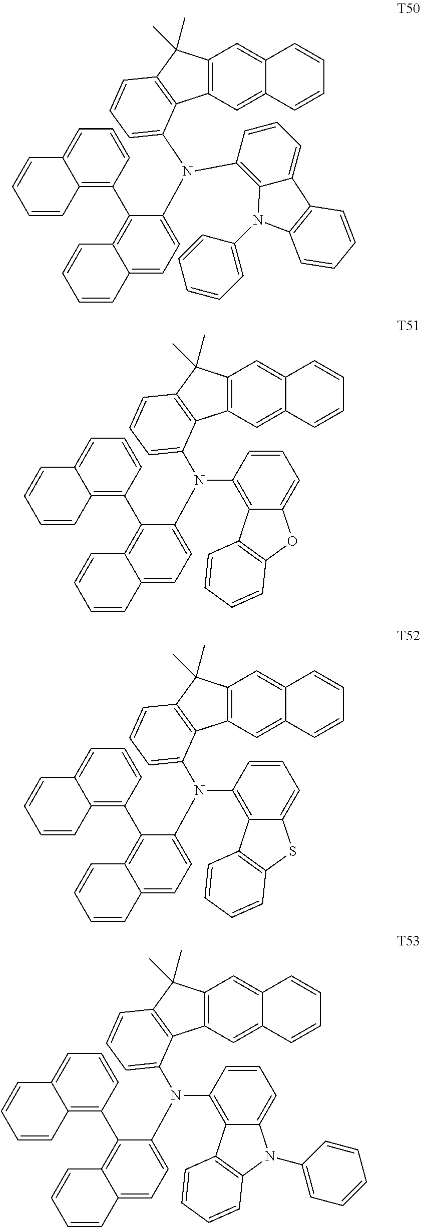

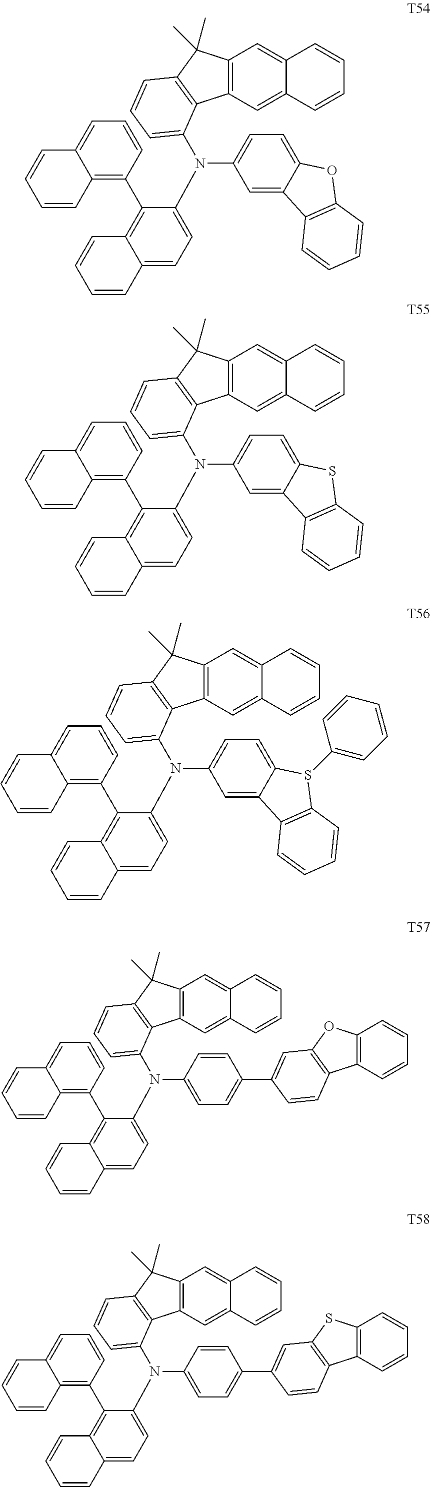

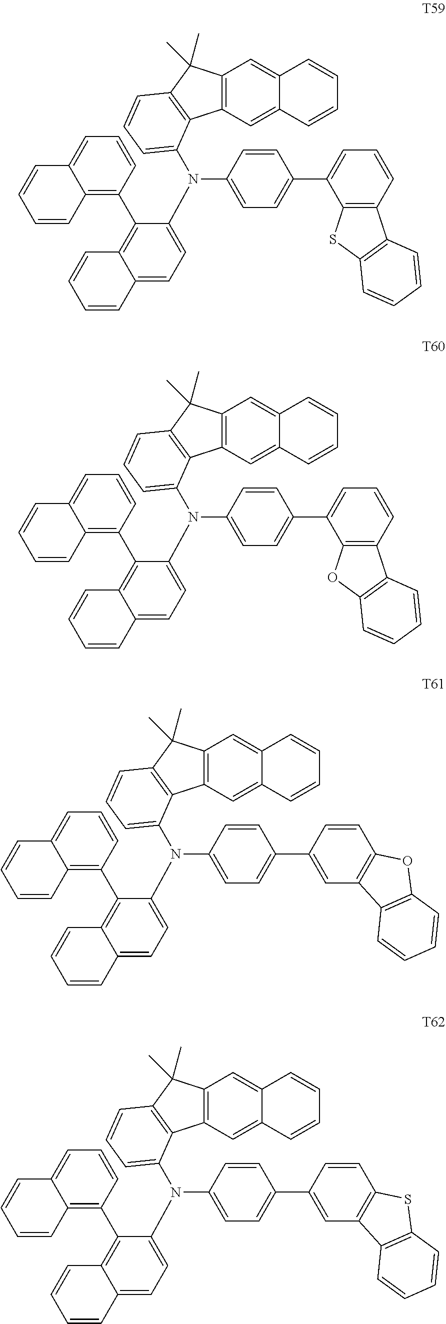 [SMILES: CC1(C)c2cc3ccccc3cc2-c2c(N(c3ccc(-c4cccc5c4sc4ccccc45)cc3)c3ccc4ccccc4c3-c3cccc4ccccc34)cccc21 ; CC1(C)c2cc3ccccc3cc2-c2c(N(c3ccc(-c4cccc5c4oc4ccccc45)cc3)c3ccc4ccccc4c3-c3cccc4ccccc34)cccc21 ; CC1(C)c2cc3ccccc3cc2-c2c(N(c3ccc(-c4ccc5oc6ccccc6c5c4)cc3)c3ccc4ccccc4c3-c3cccc4ccccc34)cccc21 ; CC1(C)c2cc3ccccc3cc2-c2c(N(c3ccc(-c4ccc5sc6ccccc6c5c4)cc3)c3ccc4ccccc4c3-c3cccc4ccccc34)cccc21]

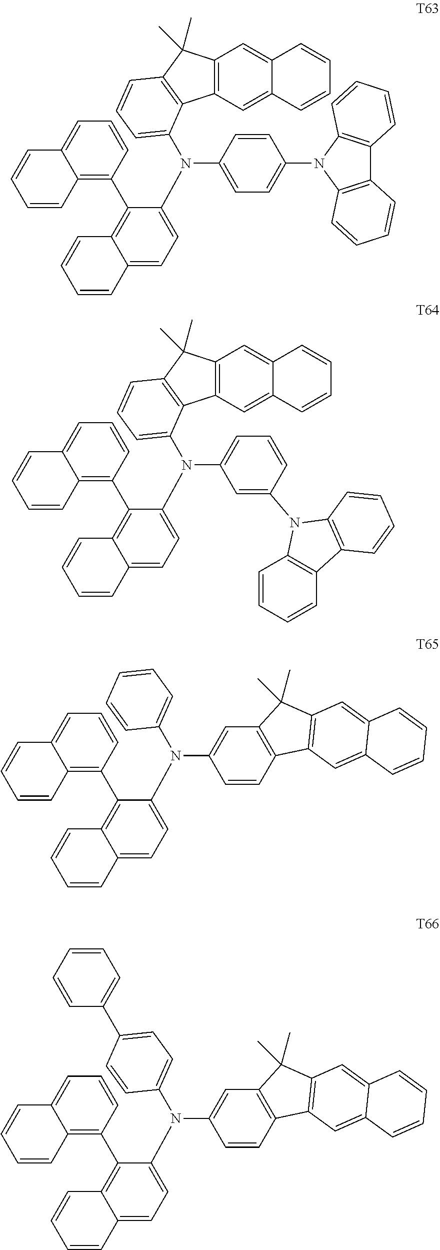 [SMILES: CC1(C)c2cc3ccccc3cc2-c2c(N(c3ccc(-n4c5ccccc5c5ccccc54)cc3)c3ccc4ccccc4c3-c3cccc4ccccc34)cccc21 ; CC1(C)c2cc3ccccc3cc2-c2c(N(c3cccc(-n4c5ccccc5c5ccccc54)c3)c3ccc4ccccc4c3-c3cccc4ccccc34)cccc21 ; CC1(C)c2cc(N(c3ccccc3)c3ccc4ccccc4c3-c3cccc4ccccc34)ccc2-c2cc3ccccc3cc21 ; CC1(C)c2cc(N(c3ccc(-c4ccccc4)cc3)c3ccc4ccccc4c3-c3cccc4ccccc34)ccc2-c2cc3ccccc3cc21]

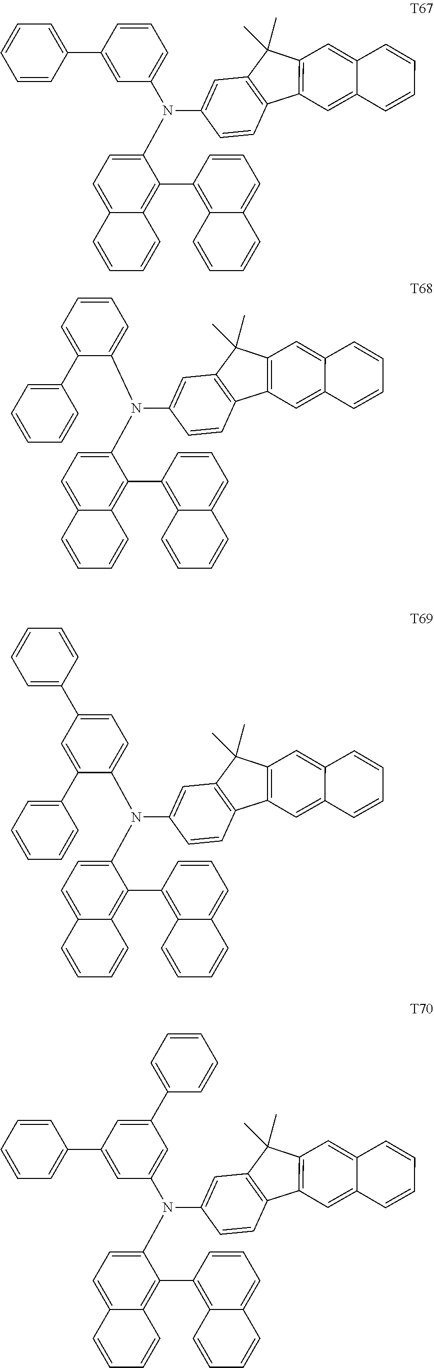

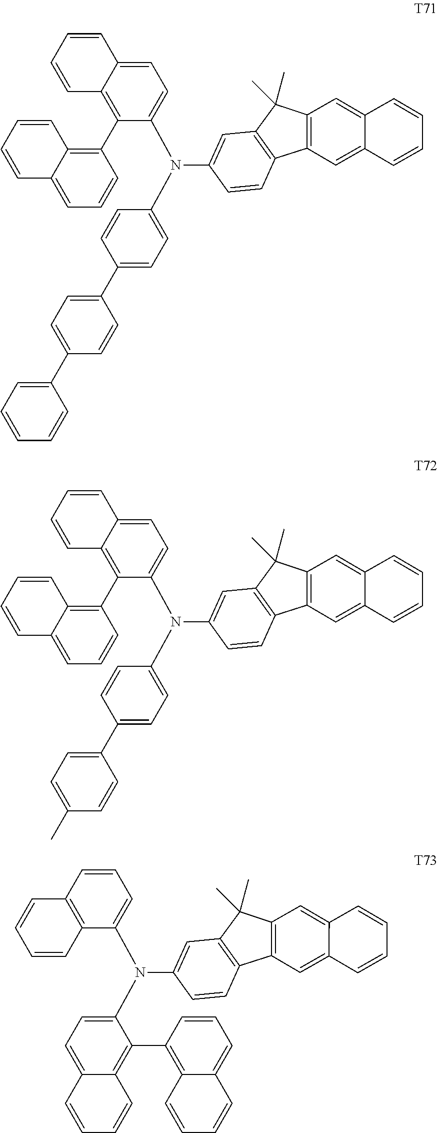

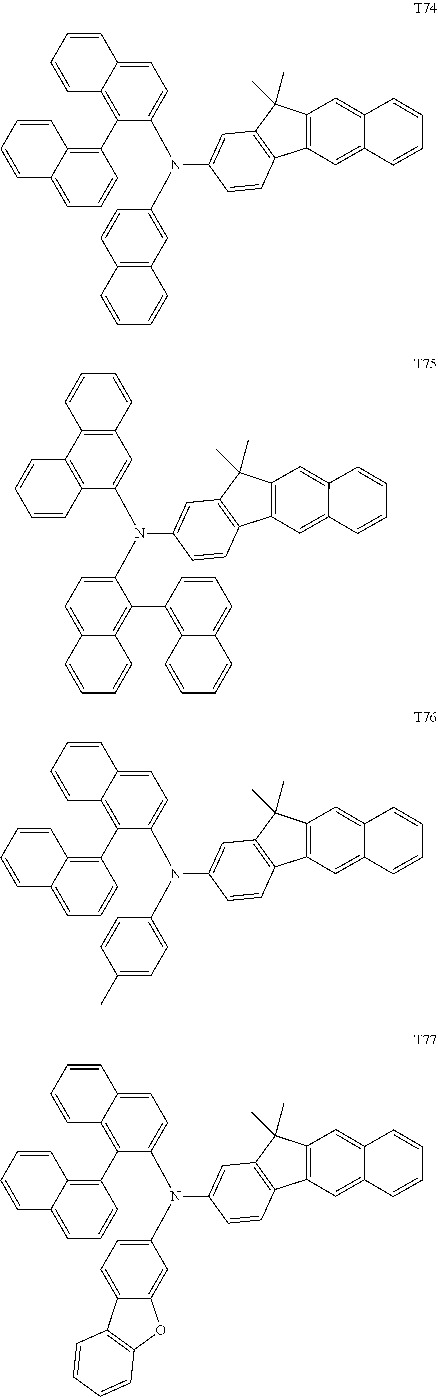 [SMILES: CC1(C)c2cc(N(c3ccc4ccccc4c3)c3ccc4ccccc4c3-c3cccc4ccccc34)ccc2-c2cc3ccccc3cc21 ; CC1(C)c2cc(N(c3ccc4ccccc4c3-c3cccc4ccccc34)c3cc4ccccc4c4ccccc34)ccc2-c2cc3ccccc3cc21 ; Cc1ccc(N(c2ccc3c(c2)C(C)(C)c2cc4ccccc4cc2-3)c2ccc3ccccc3c2-c2cccc3ccccc23)cc1 ; CC1(C)c2cc(N(c3ccc4c(c3)oc3ccccc34)c3ccc4ccccc4c3-c3cccc4ccccc34)ccc2-c2cc3ccccc3cc21]

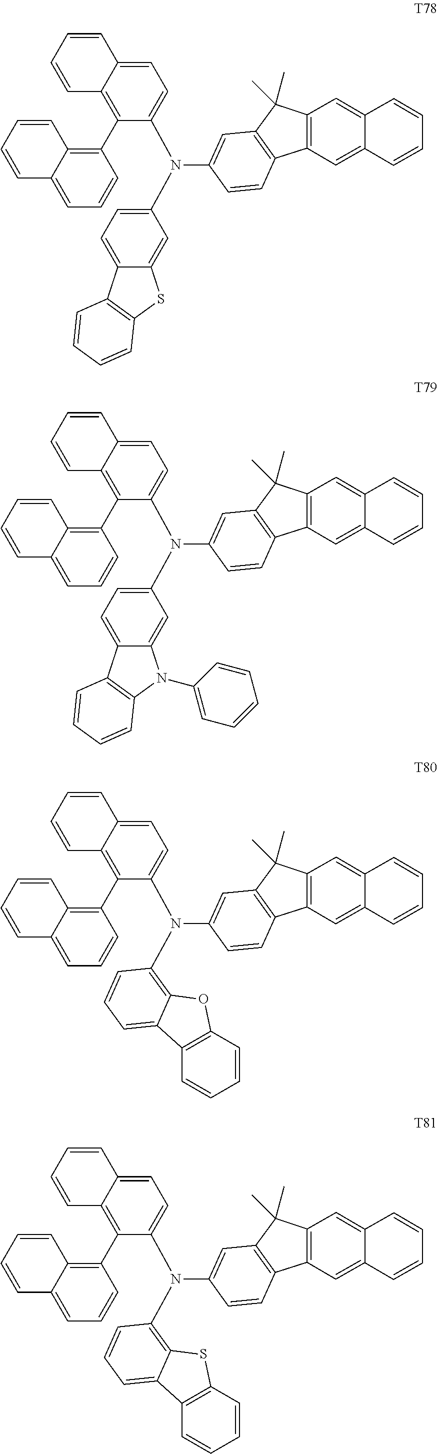 [SMILES: CC1(C)c2cc(N(c3ccc4c(c3)sc3ccccc34)c3ccc4ccccc4c3-c3cccc4ccccc34)ccc2-c2cc3ccccc3cc21 ; CC1(C)c2cc(N(c3ccc4c5ccccc5n(-c5ccccc5)c4c3)c3ccc4ccccc4c3-c3cccc4ccccc34)ccc2-c2cc3ccccc3cc21 ; CC1(C)c2cc(N(c3ccc4ccccc4c3-c3cccc4ccccc34)c3cccc4c3oc3ccccc34)ccc2-c2cc3ccccc3cc21 ; CC1(C)c2cc(N(c3ccc4ccccc4c3-c3cccc4ccccc34)c3cccc4c3sc3ccccc34)ccc2-c2cc3ccccc3cc21]

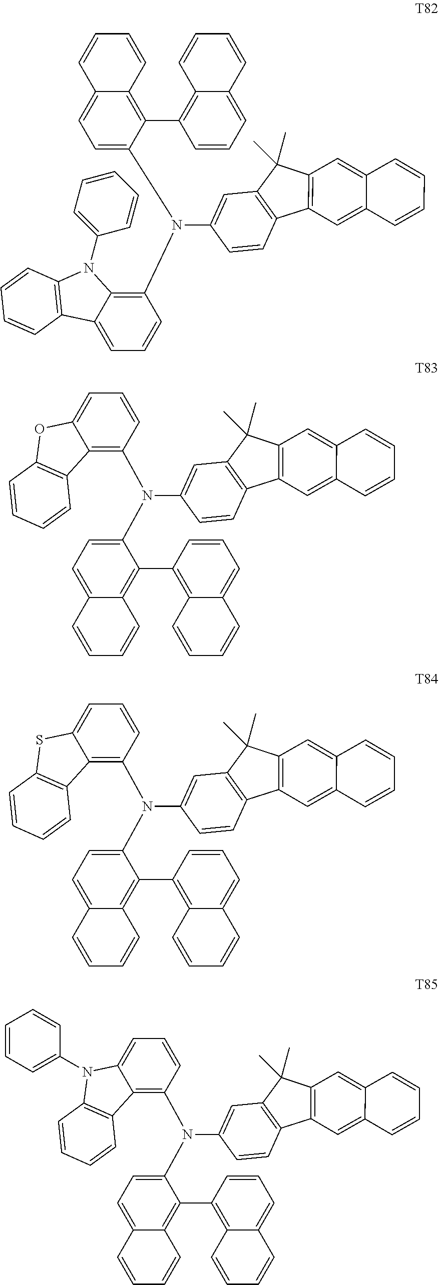

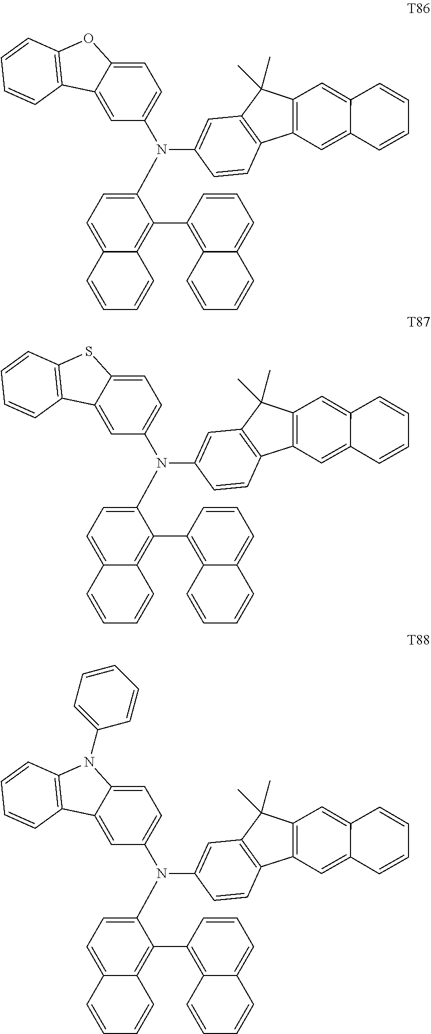

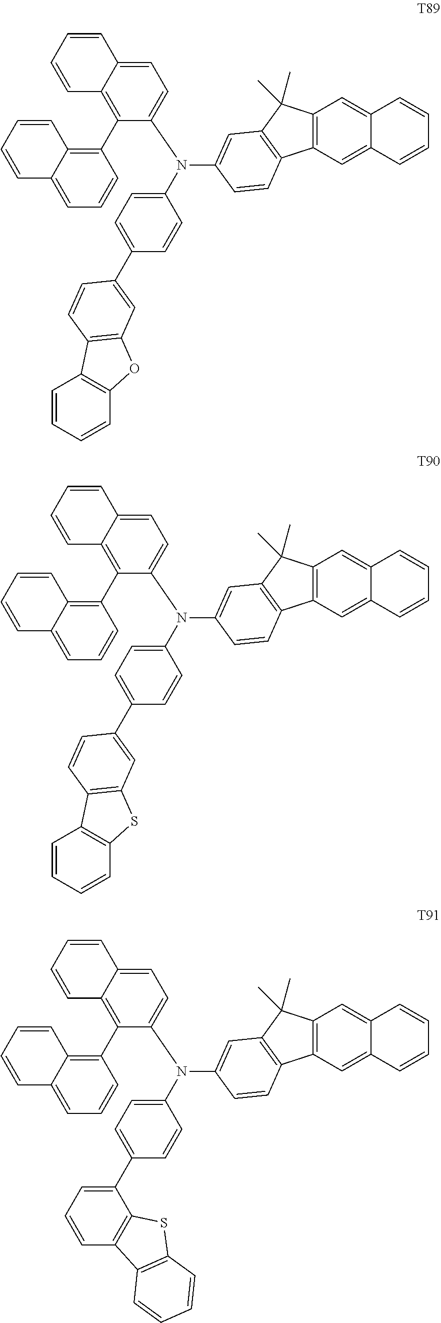 [SMILES: CC1(C)c2cc(N(c3ccc(-c4ccc5c(c4)oc4ccccc45)cc3)c3ccc4ccccc4c3-c3cccc4ccccc34)ccc2-c2cc3ccccc3cc21 ; CC1(C)c2cc(N(c3ccc(-c4ccc5c(c4)sc4ccccc45)cc3)c3ccc4ccccc4c3-c3cccc4ccccc34)ccc2-c2cc3ccccc3cc21 ; CC1(C)c2cc(N(c3ccc(-c4cccc5c4sc4ccccc45)cc3)c3ccc4ccccc4c3-c3cccc4ccccc34)ccc2-c2cc3ccccc3cc21]

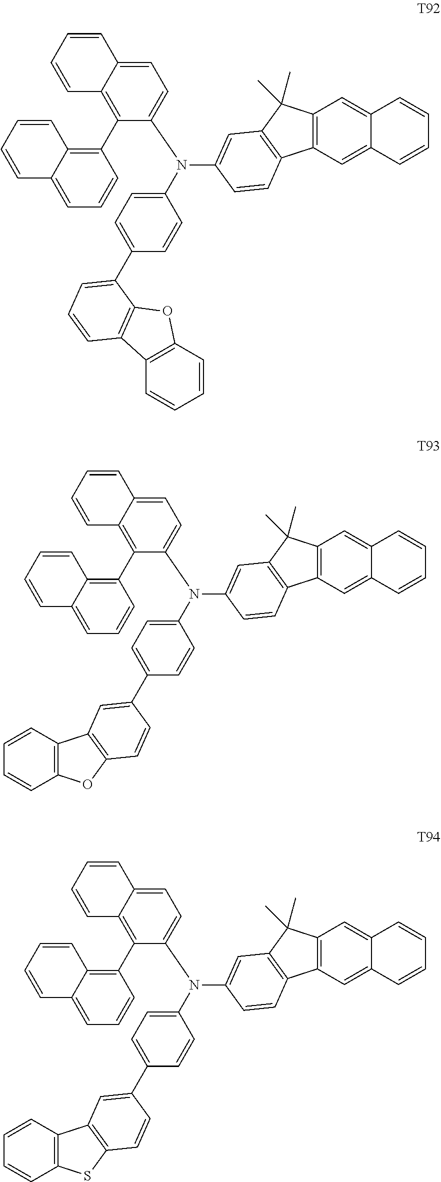 [SMILES: CC1(C)c2cc(N(c3ccc(-c4cccc5c4oc4ccccc45)cc3)c3ccc4ccccc4c3-c3cccc4ccccc34)ccc2-c2cc3ccccc3cc21 ; CC1(C)c2cc(N(c3ccc(-c4ccc5oc6ccccc6c5c4)cc3)c3ccc4ccccc4c3-c3cccc4ccccc34)ccc2-c2cc3ccccc3cc21 ; CC1(C)c2cc(N(c3ccc(-c4ccc5sc6ccccc6c5c4)cc3)c3ccc4ccccc4c3-c3cccc4ccccc34)ccc2-c2cc3ccccc3cc21]

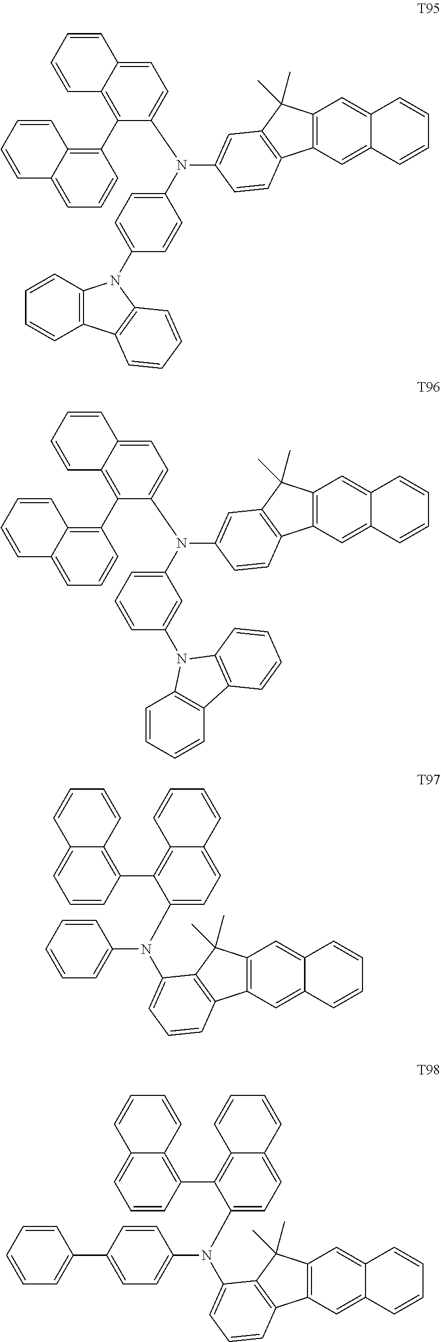

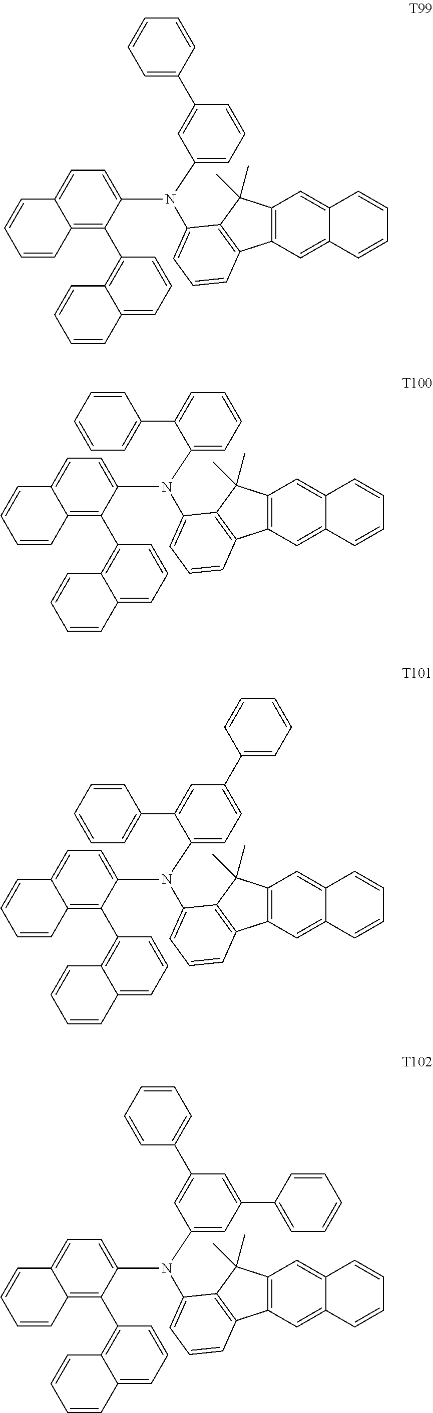 [SMILES: CC1(C)c2cc3ccccc3cc2-c2cccc(N(c3cccc(-c4ccccc4)c3)c3ccc4ccccc4c3-c3cccc4ccccc34)c21 ; CC1(C)c2cc3ccccc3cc2-c2cccc(N(c3ccccc3-c3ccccc3)c3ccc4ccccc4c3-c3cccc4ccccc34)c21 ; CC1(C)c2cc3ccccc3cc2-c2cccc(N(c3ccc(-c4ccccc4)cc3-c3ccccc3)c3ccc4ccccc4c3-c3cccc4ccccc34)c21 ; CC1(C)c2cc3ccccc3cc2-c2cccc(N(c3cc(-c4ccccc4)cc(-c4ccccc4)c3)c3ccc4ccccc4c3-c3cccc4ccccc34)c21]

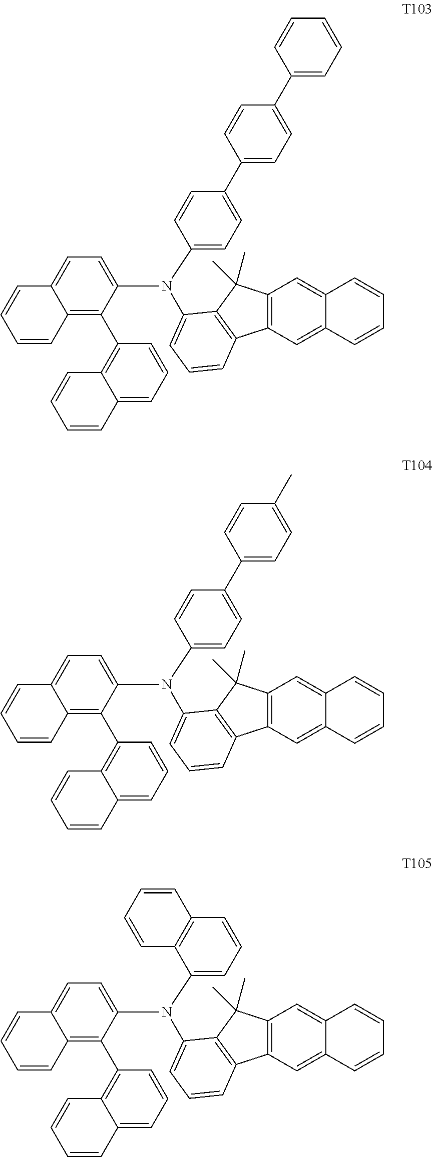 [SMILES: CC1(C)c2cc3ccccc3cc2-c2cccc(N(c3ccc(-c4ccc(-c5ccccc5)cc4)cc3)c3ccc4ccccc4c3-c3cccc4ccccc34)c21 ; Cc1ccc(-c2ccc(N(c3cccc4c3C(C)(C)c3cc5ccccc5cc3-4)c3ccc4ccccc4c3-c3cccc4ccccc34)cc2)cc1 ; CC1(C)c2cc3ccccc3cc2-c2cccc(N(c3ccc4ccccc4c3-c3cccc4ccccc34)c3cccc4ccccc34)c21]

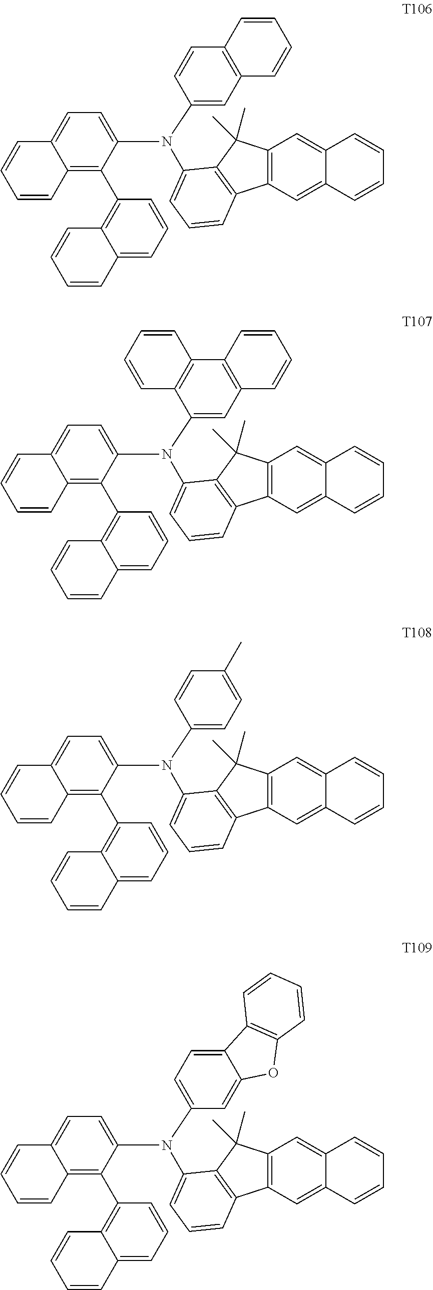 [SMILES: CC1(C)c2cc3ccccc3cc2-c2cccc(N(c3ccc4ccccc4c3)c3ccc4ccccc4c3-c3cccc4ccccc34)c21 ; CC1(C)c2cc3ccccc3cc2-c2cccc(N(c3ccc4ccccc4c3-c3cccc4ccccc34)c3cc4ccccc4c4ccccc34)c21 ; Cc1ccc(N(c2cccc3c2C(C)(C)c2cc4ccccc4cc2-3)c2ccc3ccccc3c2-c2cccc3ccccc23)cc1 ; CC1(C)c2cc3ccccc3cc2-c2cccc(N(c3ccc4c(c3)oc3ccccc34)c3ccc4ccccc4c3-c3cccc4ccccc34)c21]

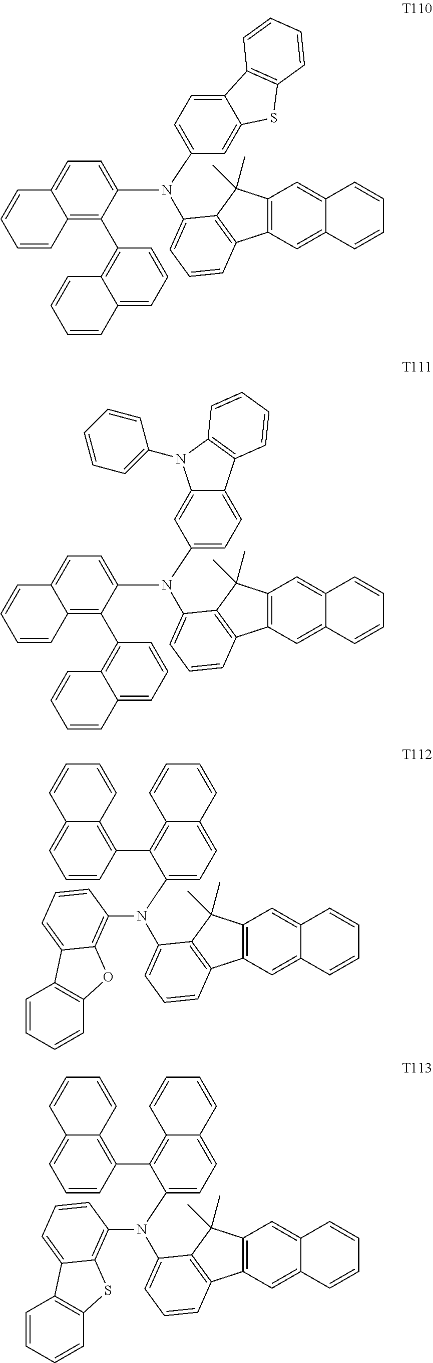

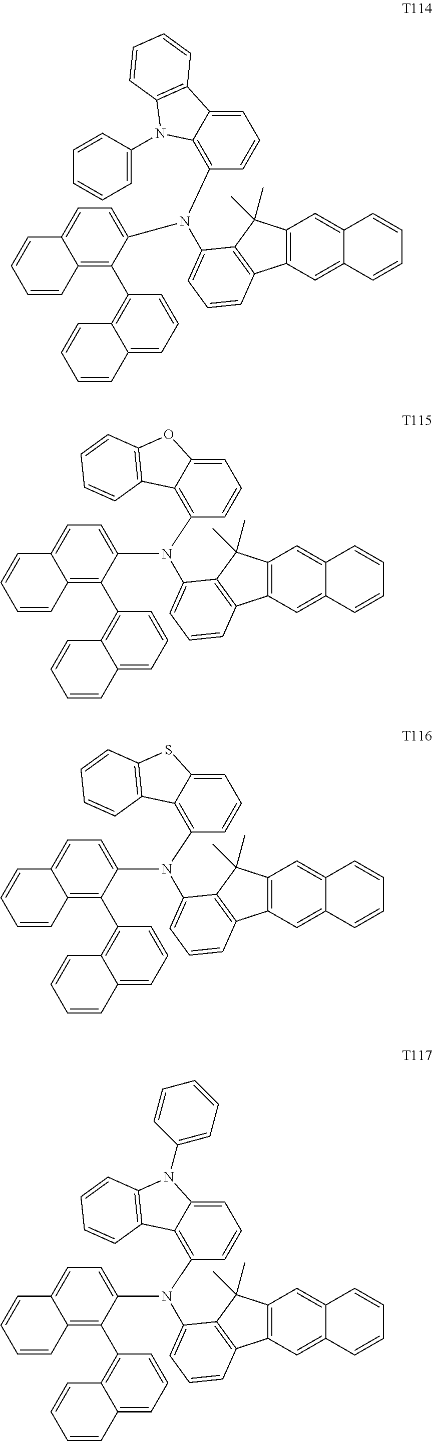 [SMILES: CC1(C)c2cc3ccccc3cc2-c2cccc(N(c3ccc4ccccc4c3-c3cccc4ccccc34)c3cccc4c5ccccc5n(-c5ccccc5)c34)c21 ; CC1(C)c2cc3ccccc3cc2-c2cccc(N(c3ccc4ccccc4c3-c3cccc4ccccc34)c3cccc4oc5ccccc5c34)c21 ; CC1(C)c2cc3ccccc3cc2-c2cccc(N(c3ccc4ccccc4c3-c3cccc4ccccc34)c3cccc4sc5ccccc5c34)c21 ; CC1(C)c2cc3ccccc3cc2-c2cccc(N(c3ccc4ccccc4c3-c3cccc4ccccc34)c3cccc4c3c3ccccc3n4-c3ccccc3)c21]

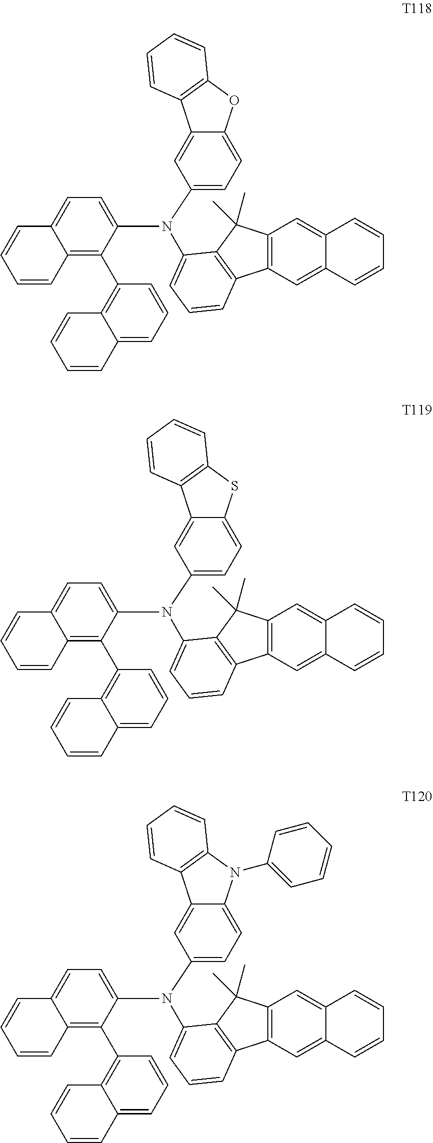 [SMILES: CC1(C)c2cc3ccccc3cc2-c2cccc(N(c3ccc4oc5ccccc5c4c3)c3ccc4ccccc4c3-c3cccc4ccccc34)c21 ; CC1(C)c2cc3ccccc3cc2-c2cccc(N(c3ccc4sc5ccccc5c4c3)c3ccc4ccccc4c3-c3cccc4ccccc34)c21 ; CC1(C)c2cc3ccccc3cc2-c2cccc(N(c3ccc4c(c3)c3ccccc3n4-c3ccccc3)c3ccc4ccccc4c3-c3cccc4ccccc34)c21]

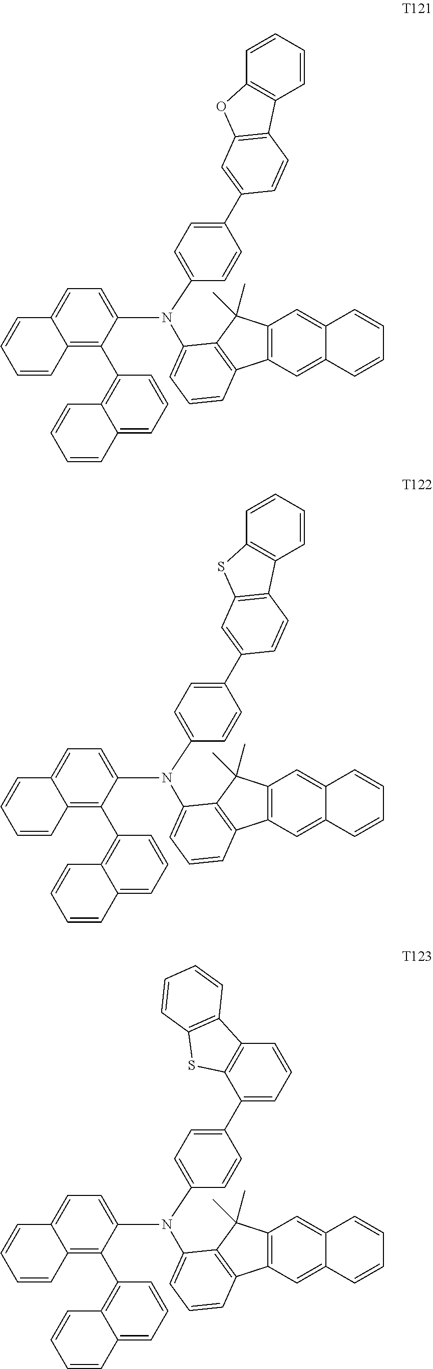 [SMILES: CC1(C)c2cc3ccccc3cc2-c2cccc(N(c3ccc(-c4ccc5c(c4)oc4ccccc45)cc3)c3ccc4ccccc4c3-c3cccc4ccccc34)c21 ; CC1(C)c2cc3ccccc3cc2-c2cccc(N(c3ccc(-c4ccc5c(c4)sc4ccccc45)cc3)c3ccc4ccccc4c3-c3cccc4ccccc34)c21 ; CC1(C)c2cc3ccccc3cc2-c2cccc(N(c3ccc(-c4cccc5c4sc4ccccc45)cc3)c3ccc4ccccc4c3-c3cccc4ccccc34)c21]

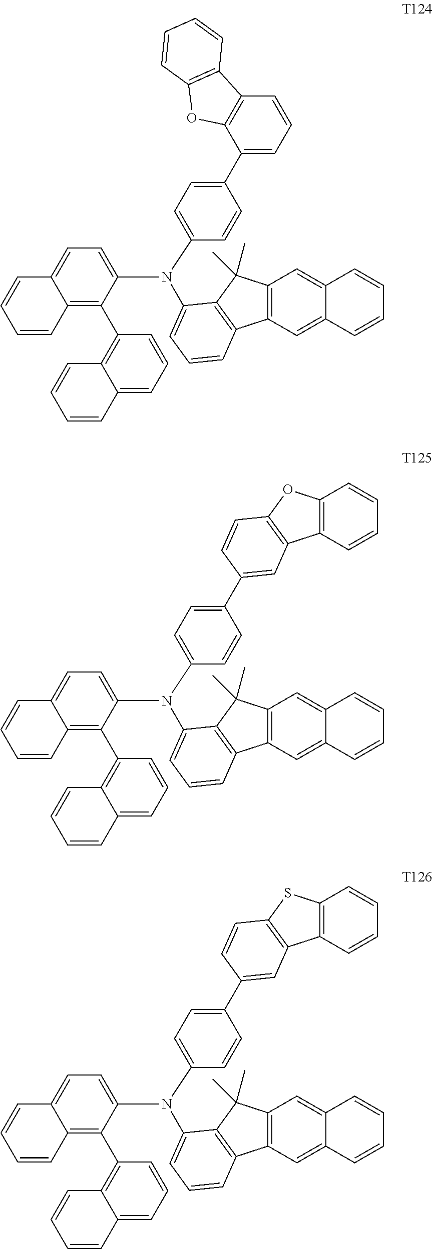 [SMILES: CC1(C)c2cc3ccccc3cc2-c2cccc(N(c3ccc(-c4cccc5c4oc4ccccc45)cc3)c3ccc4ccccc4c3-c3cccc4ccccc34)c21 ; CC1(C)c2cc3ccccc3cc2-c2cccc(N(c3ccc(-c4ccc5oc6ccccc6c5c4)cc3)c3ccc4ccccc4c3-c3cccc4ccccc34)c21 ; CC1(C)c2cc3ccccc3cc2-c2cccc(N(c3ccc(-c4ccc5sc6ccccc6c5c4)cc3)c3ccc4ccccc4c3-c3cccc4ccccc34)c21]

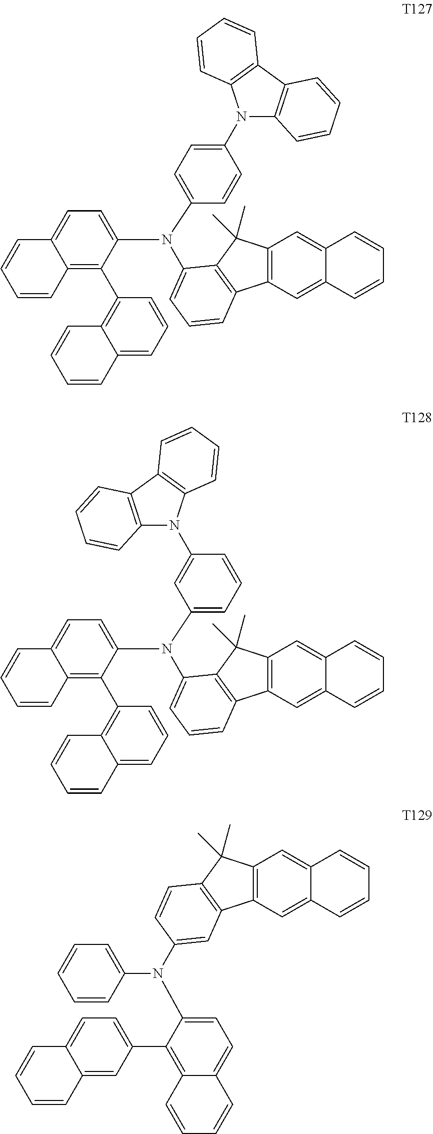 [SMILES: CC1(C)c2cc3ccccc3cc2-c2cccc(N(c3ccc(-n4c5ccccc5c5ccccc54)cc3)c3ccc4ccccc4c3-c3cccc4ccccc34)c21 ; CC1(C)c2cc3ccccc3cc2-c2cccc(N(c3cccc(-n4c5ccccc5c5ccccc54)c3)c3ccc4ccccc4c3-c3cccc4ccccc34)c21 ; CC1(C)c2ccc(N(c3ccccc3)c3ccc4ccccc4c3-c3ccc4ccccc4c3)cc2-c2cc3ccccc3cc21]

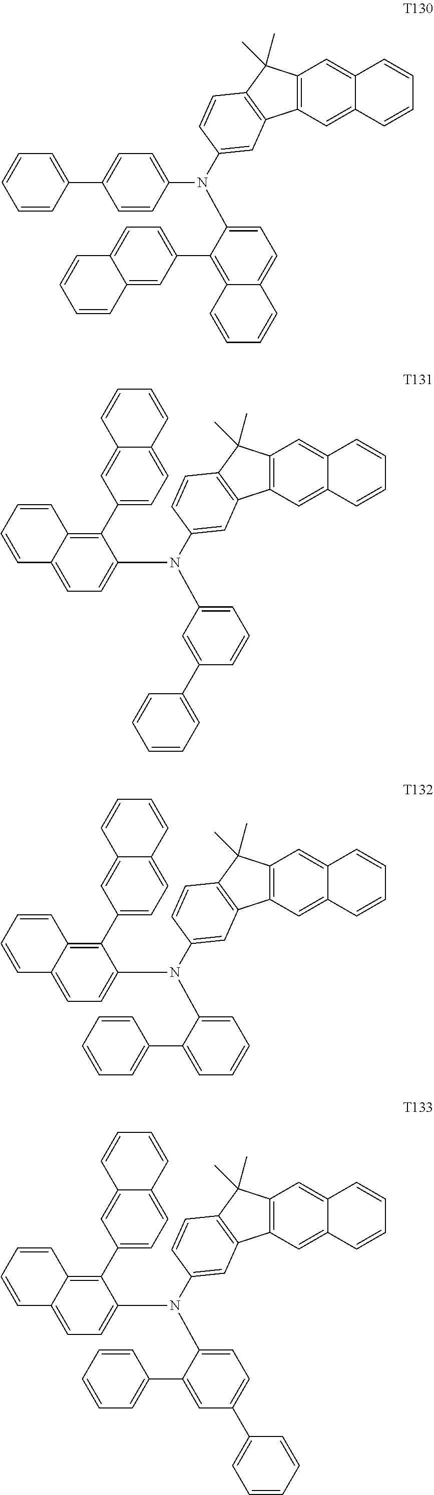 [SMILES: CC1(C)c2ccc(N(c3ccc(-c4ccccc4)cc3)c3ccc4ccccc4c3-c3ccc4ccccc4c3)cc2-c2cc3ccccc3cc21 ; CC1(C)c2ccc(N(c3cccc(-c4ccccc4)c3)c3ccc4ccccc4c3-c3ccc4ccccc4c3)cc2-c2cc3ccccc3cc21 ; CC1(C)c2ccc(N(c3ccccc3-c3ccccc3)c3ccc4ccccc4c3-c3ccc4ccccc4c3)cc2-c2cc3ccccc3cc21 ; CC1(C)c2ccc(N(c3ccc(-c4ccccc4)cc3-c3ccccc3)c3ccc4ccccc4c3-c3ccc4ccccc4c3)cc2-c2cc3ccccc3cc21]

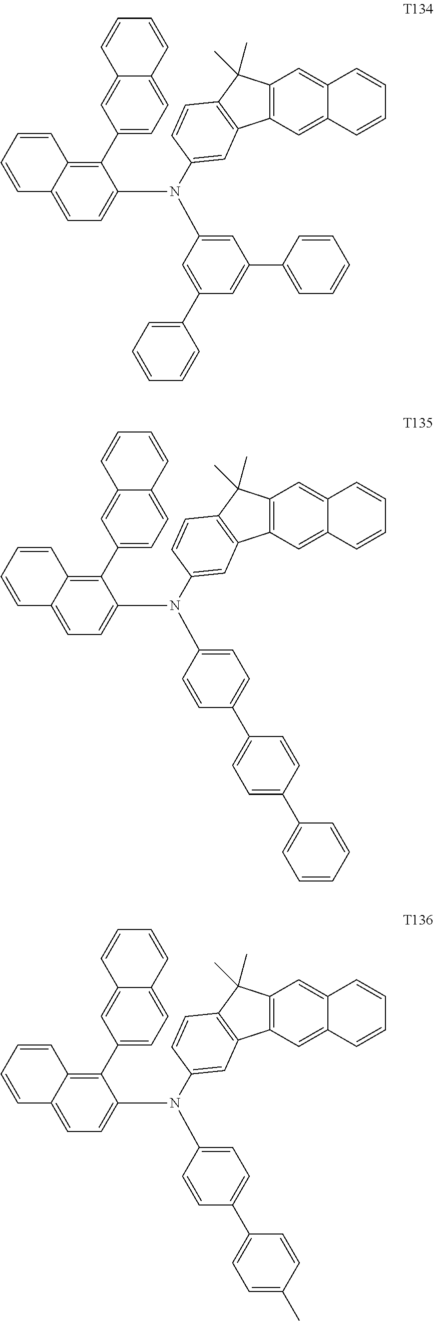 [SMILES: CC1(C)c2ccc(N(c3cc(-c4ccccc4)cc(-c4ccccc4)c3)c3ccc4ccccc4c3-c3ccc4ccccc4c3)cc2-c2cc3ccccc3cc21 ; CC1(C)c2ccc(N(c3ccc(-c4ccc(-c5ccccc5)cc4)cc3)c3ccc4ccccc4c3-c3ccc4ccccc4c3)cc2-c2cc3ccccc3cc21 ; Cc1ccc(-c2ccc(N(c3ccc4c(c3)-c3cc5ccccc5cc3C4(C)C)c3ccc4ccccc4c3-c3ccc4ccccc4c3)cc2)cc1]

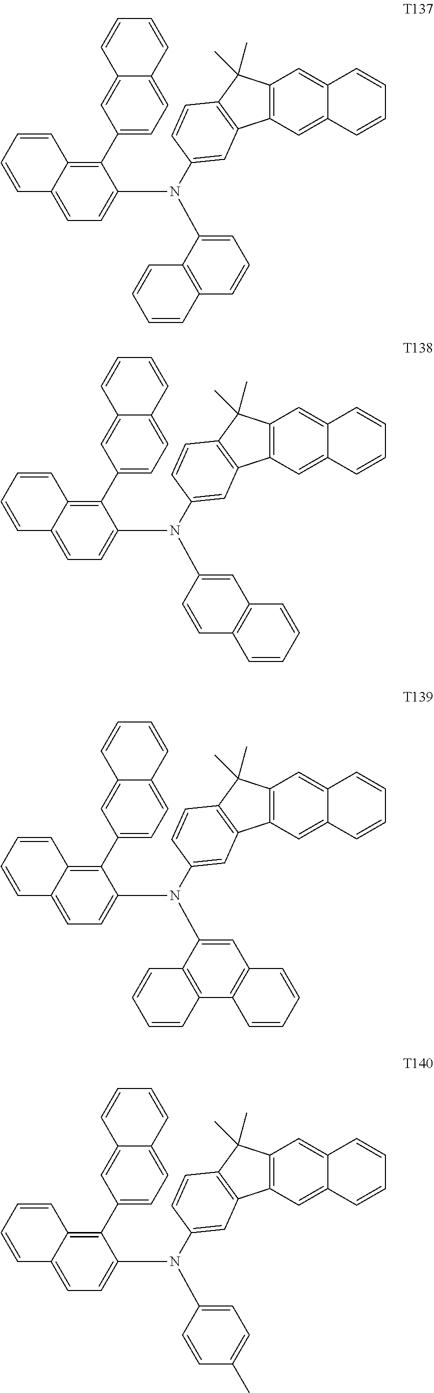

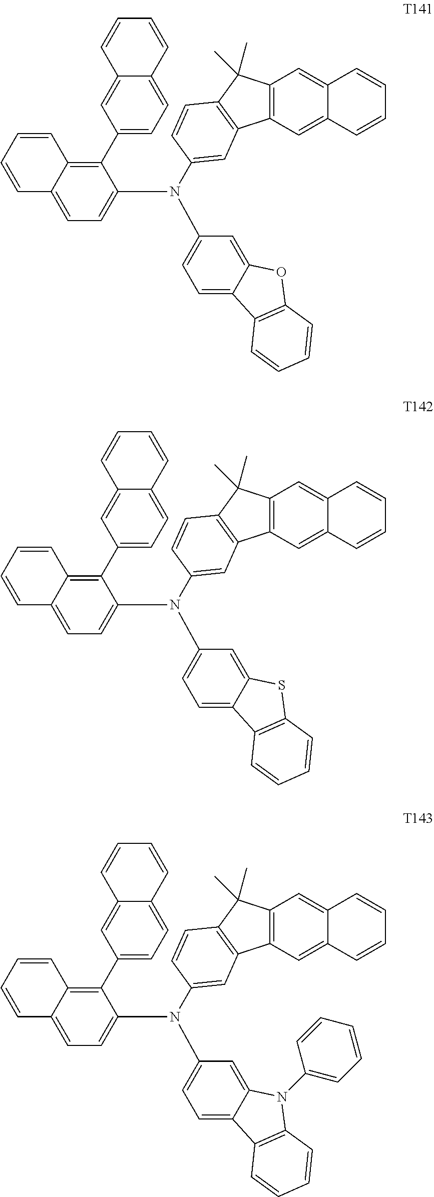 [SMILES: CC1(C)c2ccc(N(c3ccc4c(c3)oc3ccccc34)c3ccc4ccccc4c3-c3ccc4ccccc4c3)cc2-c2cc3ccccc3cc21 ; CC1(C)c2ccc(N(c3ccc4c(c3)sc3ccccc34)c3ccc4ccccc4c3-c3ccc4ccccc4c3)cc2-c2cc3ccccc3cc21 ; CC1(C)c2ccc(N(c3ccc4c5ccccc5n(-c5ccccc5)c4c3)c3ccc4ccccc4c3-c3ccc4ccccc4c3)cc2-c2cc3ccccc3cc21]

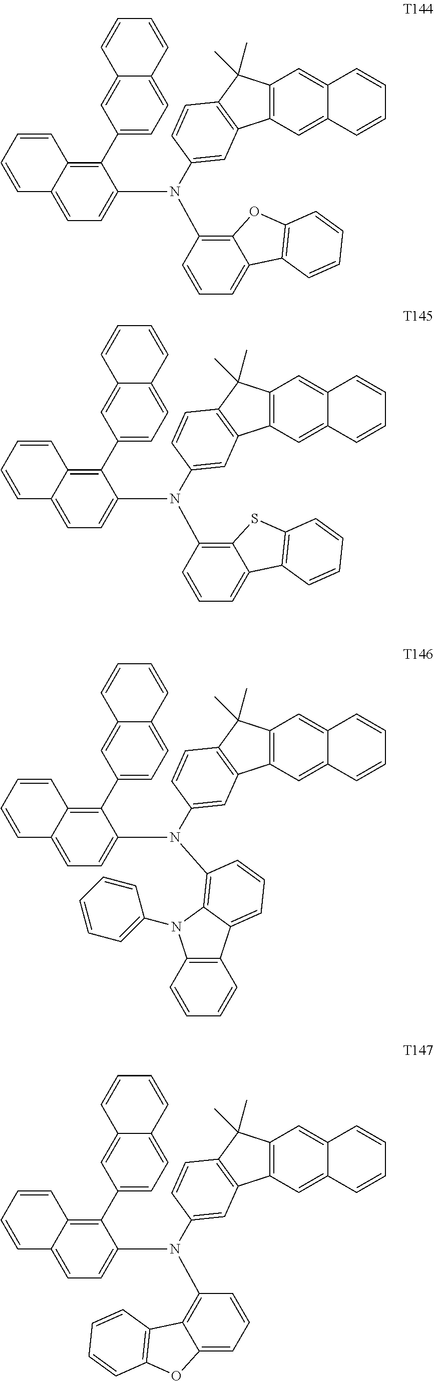 [SMILES: CC1(C)c2ccc(N(c3ccc4ccccc4c3-c3ccc4ccccc4c3)c3cccc4c3oc3ccccc34)cc2-c2cc3ccccc3cc21 ; CC1(C)c2ccc(N(c3ccc4ccccc4c3-c3ccc4ccccc4c3)c3cccc4c3sc3ccccc34)cc2-c2cc3ccccc3cc21 ; CC1(C)c2ccc(N(c3ccc4ccccc4c3-c3ccc4ccccc4c3)c3cccc4c5ccccc5n(-c5ccccc5)c34)cc2-c2cc3ccccc3cc21 ; CC1(C)c2ccc(N(c3ccc4ccccc4c3-c3ccc4ccccc4c3)c3cccc4oc5ccccc5c34)cc2-c2cc3ccccc3cc21]

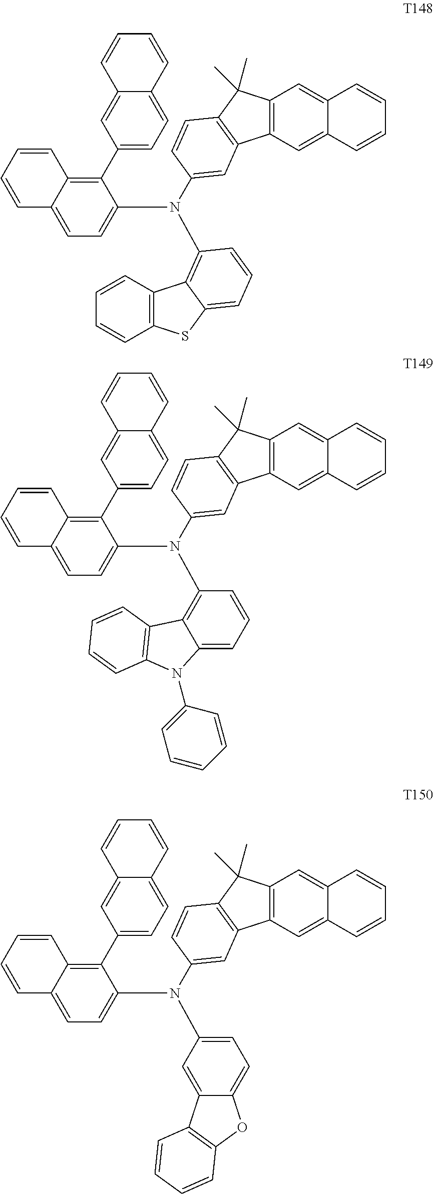

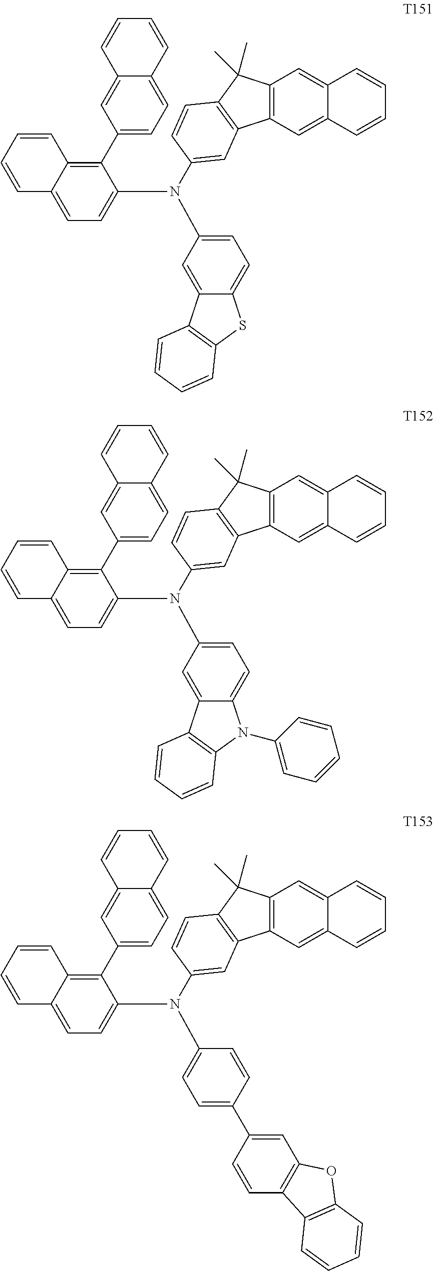

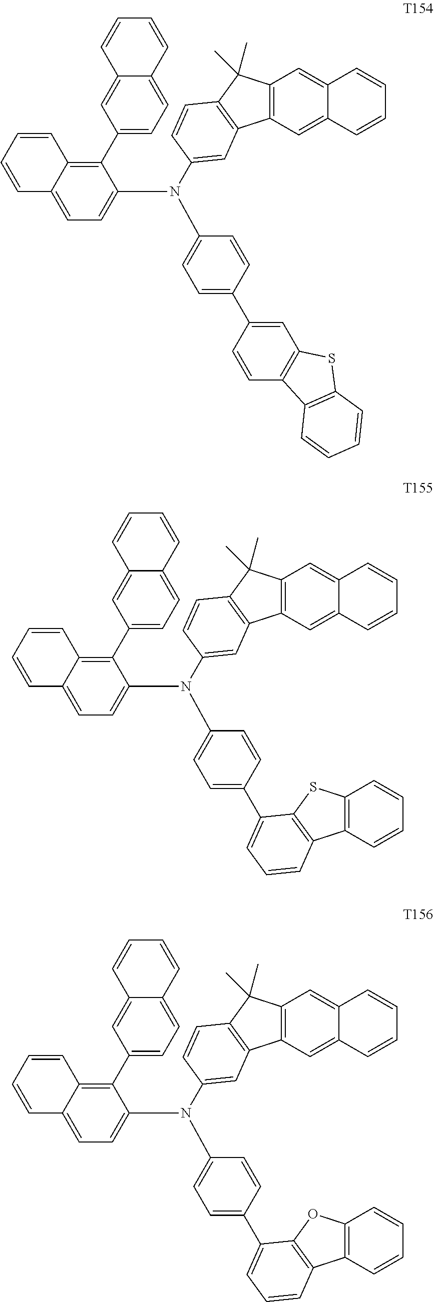 [SMILES: CC1(C)c2ccc(N(c3ccc(-c4ccc5c(c4)sc4ccccc45)cc3)c3ccc4ccccc4c3-c3ccc4ccccc4c3)cc2-c2cc3ccccc3cc21 ; CC1(C)c2ccc(N(c3ccc(-c4cccc5c4sc4ccccc45)cc3)c3ccc4ccccc4c3-c3ccc4ccccc4c3)cc2-c2cc3ccccc3cc21 ; CC1(C)c2ccc(N(c3ccc(-c4cccc5c4oc4ccccc45)cc3)c3ccc4ccccc4c3-c3ccc4ccccc4c3)cc2-c2cc3ccccc3cc21]

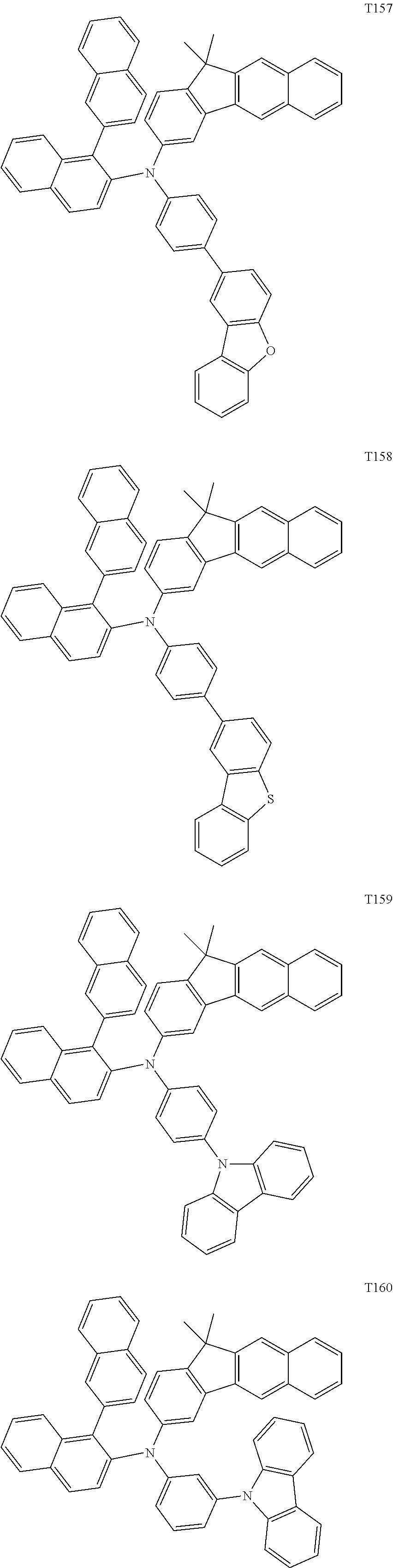 [SMILES: CC1(C)c2ccc(N(c3ccc(-c4ccc5oc6ccccc6c5c4)cc3)c3ccc4ccccc4c3-c3ccc4ccccc4c3)cc2-c2cc3ccccc3cc21 ; CC1(C)c2ccc(N(c3ccc(-c4ccc5sc6ccccc6c5c4)cc3)c3ccc4ccccc4c3-c3ccc4ccccc4c3)cc2-c2cc3ccccc3cc21 ; CC1(C)c2ccc(N(c3ccc(-n4c5ccccc5c5ccccc54)cc3)c3ccc4ccccc4c3-c3ccc4ccccc4c3)cc2-c2cc3ccccc3cc21 ; CC1(C)c2ccc(N(c3cccc(-n4c5ccccc5c5ccccc54)c3)c3ccc4ccccc4c3-c3ccc4ccccc4c3)cc2-c2cc3ccccc3cc21]

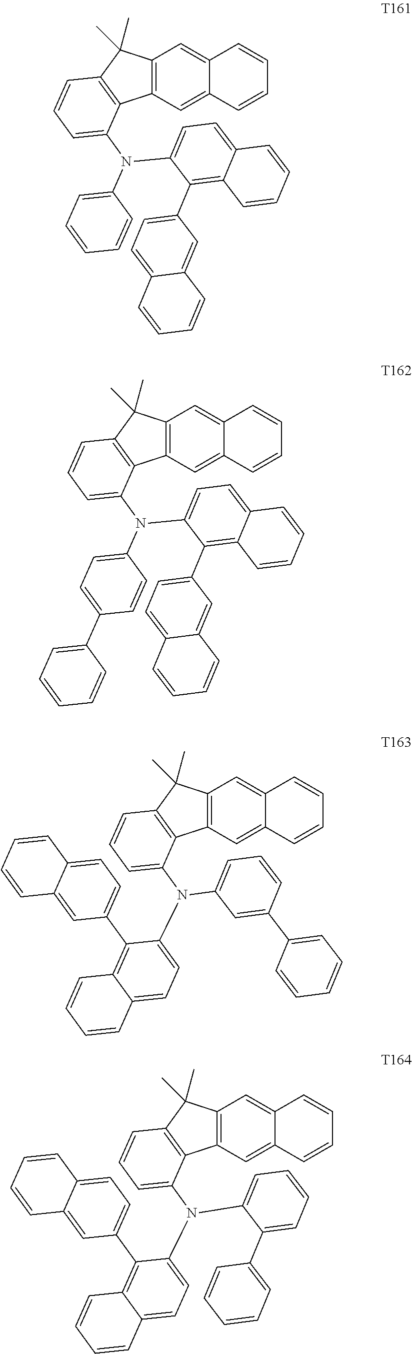 [SMILES: CC1(C)c2cc3ccccc3cc2-c2c(N(c3ccccc3)c3ccc4ccccc4c3-c3ccc4ccccc4c3)cccc21 ; CC1(C)c2cc3ccccc3cc2-c2c(N(c3ccc(-c4ccccc4)cc3)c3ccc4ccccc4c3-c3ccc4ccccc4c3)cccc21 ; CC1(C)c2cc3ccccc3cc2-c2c(N(c3cccc(-c4ccccc4)c3)c3ccc4ccccc4c3-c3ccc4ccccc4c3)cccc21 ; CC1(C)c2cc3ccccc3cc2-c2c(N(c3ccccc3-c3ccccc3)c3ccc4ccccc4c3-c3ccc4ccccc4c3)cccc21]

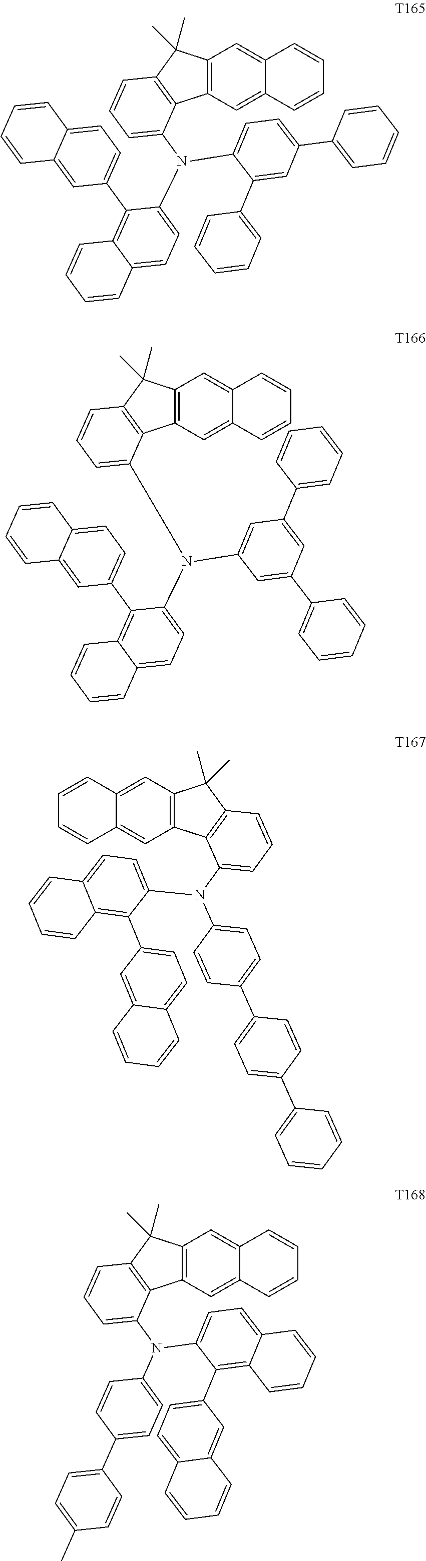 [SMILES: CC1(C)c2cc3ccccc3cc2-c2c(N(c3ccc(-c4ccccc4)cc3-c3ccccc3)c3ccc4ccccc4c3-c3ccc4ccccc4c3)cccc21 ; CC1(C)c2cc3ccccc3cc2-c2c(N(c3cc(-c4ccccc4)cc(-c4ccccc4)c3)c3ccc4ccccc4c3-c3ccc4ccccc4c3)cccc21 ; CC1(C)c2cc3ccccc3cc2-c2c(N(c3ccc(-c4ccc(-c5ccccc5)cc4)cc3)c3ccc4ccccc4c3-c3ccc4ccccc4c3)cccc21 ; Cc1ccc(-c2ccc(N(c3cccc4c3-c3cc5ccccc5cc3C4(C)C)c3ccc4ccccc4c3-c3ccc4ccccc4c3)cc2)cc1]

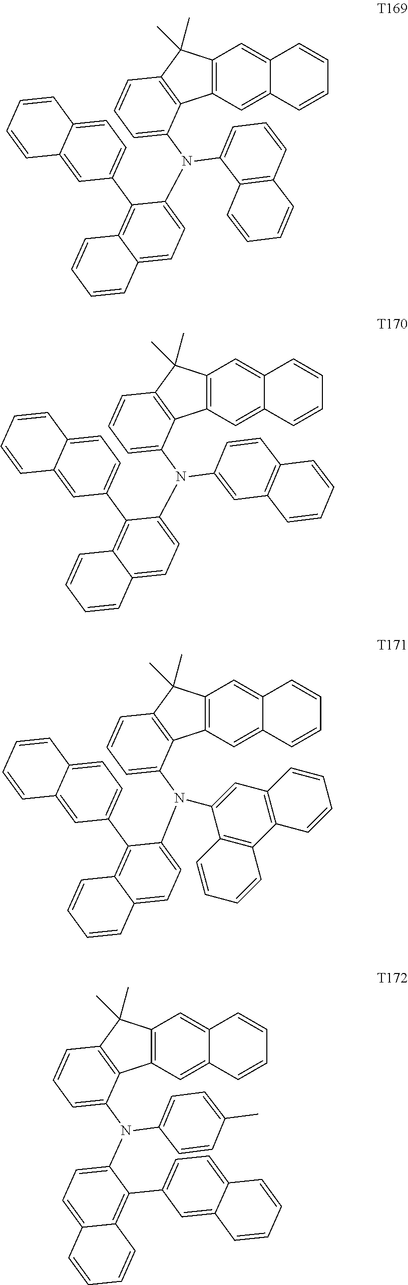 [SMILES: CC1(C)c2cc3ccccc3cc2-c2c(N(c3ccc4ccccc4c3-c3ccc4ccccc4c3)c3cccc4ccccc34)cccc21 ; CC1(C)c2cc3ccccc3cc2-c2c(N(c3ccc4ccccc4c3)c3ccc4ccccc4c3-c3ccc4ccccc4c3)cccc21 ; CC1(C)c2cc3ccccc3cc2-c2c(N(c3ccc4ccccc4c3-c3ccc4ccccc4c3)c3cc4ccccc4c4ccccc34)cccc21 ; Cc1ccc(N(c2cccc3c2-c2cc4ccccc4cc2C3(C)C)c2ccc3ccccc3c2-c2ccc3ccccc3c2)cc1]

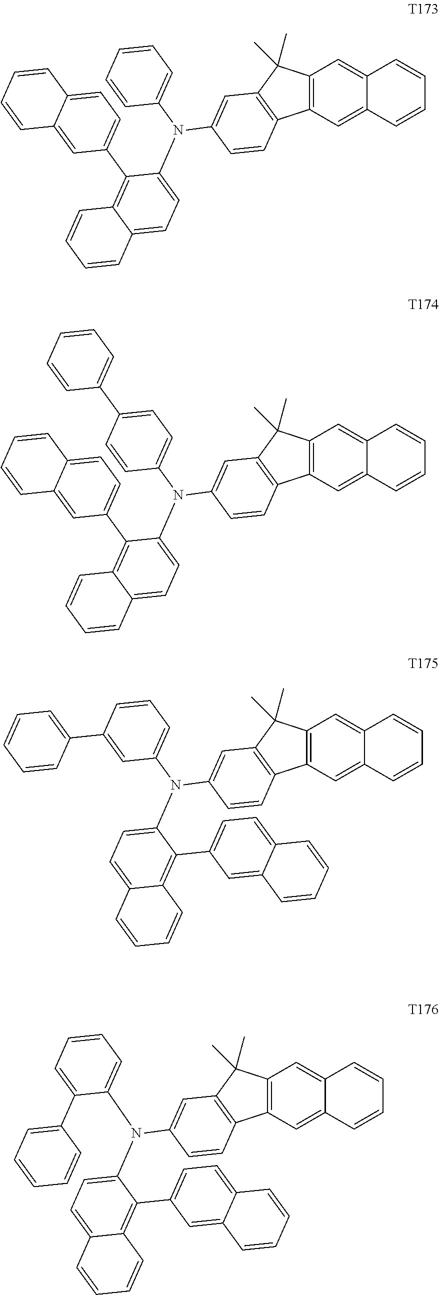 [SMILES: CC1(C)c2cc(N(c3ccccc3)c3ccc4ccccc4c3-c3ccc4ccccc4c3)ccc2-c2cc3ccccc3cc21 ; CC1(C)c2cc(N(c3ccc(-c4ccccc4)cc3)c3ccc4ccccc4c3-c3ccc4ccccc4c3)ccc2-c2cc3ccccc3cc21 ; CC1(C)c2cc(N(c3cccc(-c4ccccc4)c3)c3ccc4ccccc4c3-c3ccc4ccccc4c3)ccc2-c2cc3ccccc3cc21 ; CC1(C)c2cc(N(c3ccccc3-c3ccccc3)c3ccc4ccccc4c3-c3ccc4ccccc4c3)ccc2-c2cc3ccccc3cc21]

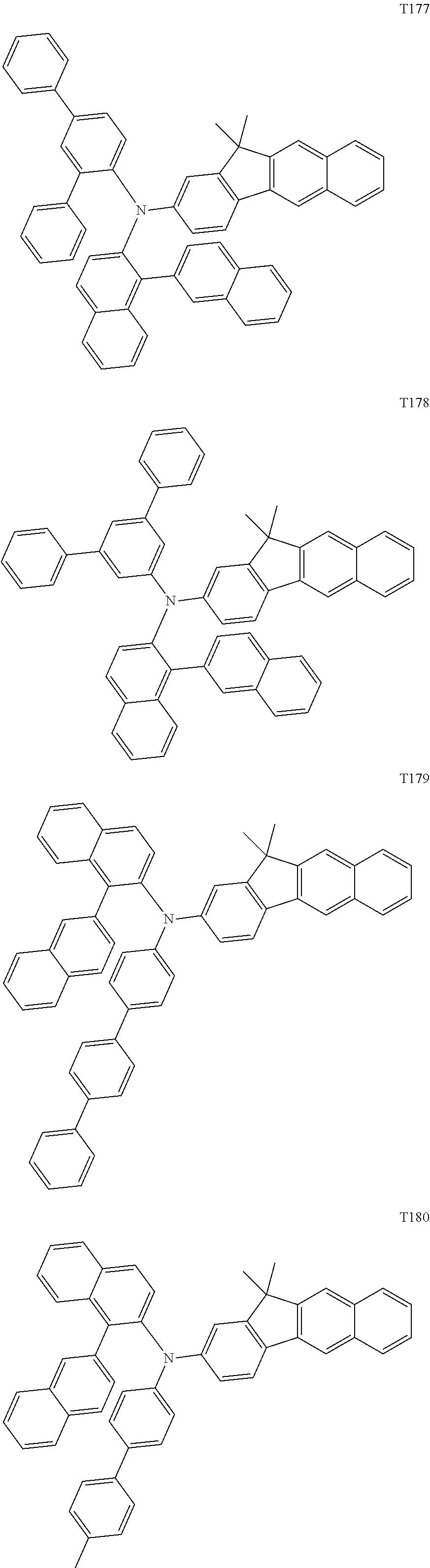 [SMILES: CC1(C)c2cc(N(c3ccc(-c4ccccc4)cc3-c3ccccc3)c3ccc4ccccc4c3-c3ccc4ccccc4c3)ccc2-c2cc3ccccc3cc21 ; CC1(C)c2cc(N(c3cc(-c4ccccc4)cc(-c4ccccc4)c3)c3ccc4ccccc4c3-c3ccc4ccccc4c3)ccc2-c2cc3ccccc3cc21 ; CC1(C)c2cc(N(c3ccc(-c4ccc(-c5ccccc5)cc4)cc3)c3ccc4ccccc4c3-c3ccc4ccccc4c3)ccc2-c2cc3ccccc3cc21 ; Cc1ccc(-c2ccc(N(c3ccc4c(c3)C(C)(C)c3cc5ccccc5cc3-4)c3ccc4ccccc4c3-c3ccc4ccccc4c3)cc2)cc1]

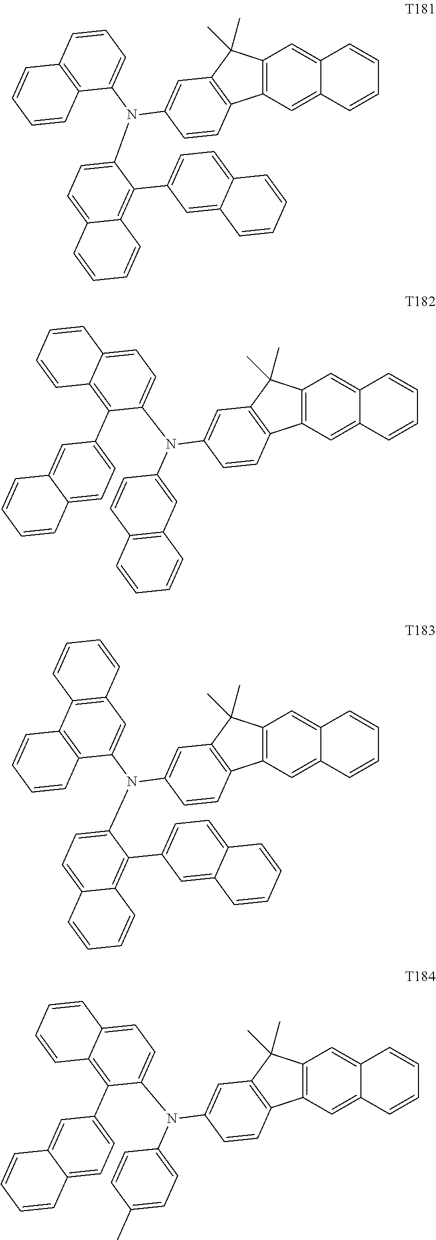 [SMILES: CC1(C)c2cc(N(c3ccc4ccccc4c3-c3ccc4ccccc4c3)c3cccc4ccccc34)ccc2-c2cc3ccccc3cc21 ; CC1(C)c2cc(N(c3ccc4ccccc4c3)c3ccc4ccccc4c3-c3ccc4ccccc4c3)ccc2-c2cc3ccccc3cc21 ; CC1(C)c2cc(N(c3ccc4ccccc4c3-c3ccc4ccccc4c3)c3cc4ccccc4c4ccccc34)ccc2-c2cc3ccccc3cc21 ; Cc1ccc(N(c2ccc3c(c2)C(C)(C)c2cc4ccccc4cc2-3)c2ccc3ccccc3c2-c2ccc3ccccc3c2)cc1]

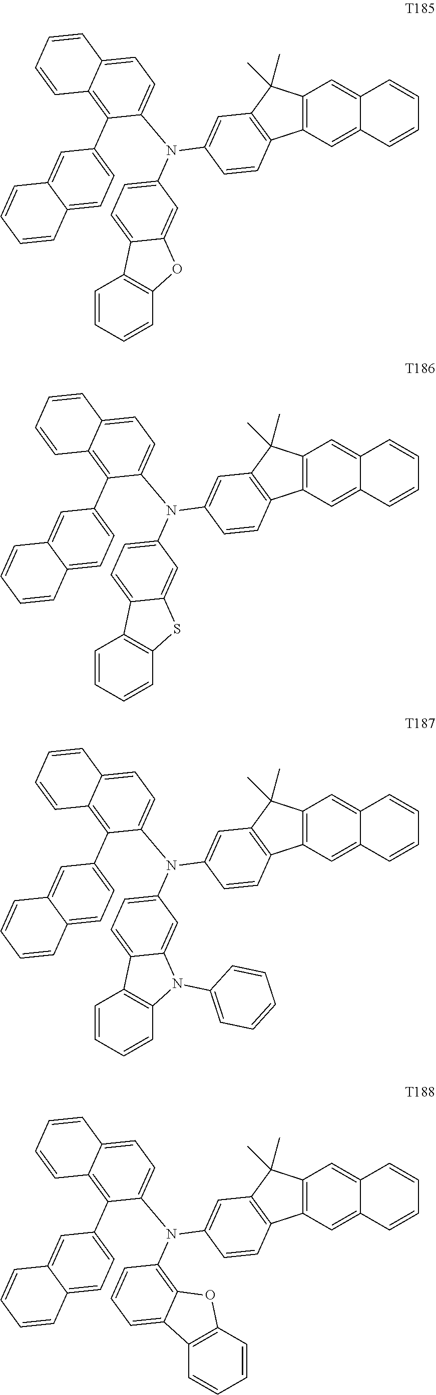 [SMILES: CC1(C)c2cc(N(c3ccc4c(c3)oc3ccccc34)c3ccc4ccccc4c3-c3ccc4ccccc4c3)ccc2-c2cc3ccccc3cc21 ; CC1(C)c2cc(N(c3ccc4c(c3)sc3ccccc34)c3ccc4ccccc4c3-c3ccc4ccccc4c3)ccc2-c2cc3ccccc3cc21 ; CC1(C)c2cc(N(c3ccc4c5ccccc5n(-c5ccccc5)c4c3)c3ccc4ccccc4c3-c3ccc4ccccc4c3)ccc2-c2cc3ccccc3cc21 ; CC1(C)c2cc(N(c3ccc4ccccc4c3-c3ccc4ccccc4c3)c3cccc4c3oc3ccccc34)ccc2-c2cc3ccccc3cc21]

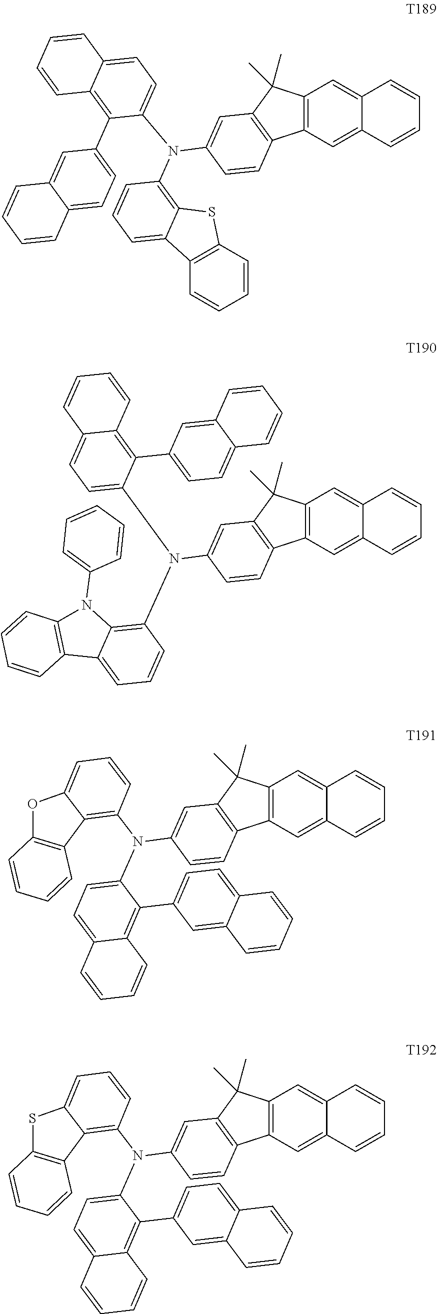 [SMILES: CC1(C)c2cc(N(c3ccc4ccccc4c3-c3ccc4ccccc4c3)c3cccc4c3sc3ccccc34)ccc2-c2cc3ccccc3cc21 ; CC1(C)c2cc(N(c3ccc4ccccc4c3-c3ccc4ccccc4c3)c3cccc4c5ccccc5n(-c5ccccc5)c34)ccc2-c2cc3ccccc3cc21 ; CC1(C)c2cc(N(c3ccc4ccccc4c3-c3ccc4ccccc4c3)c3cccc4oc5ccccc5c34)ccc2-c2cc3ccccc3cc21 ; CC1(C)c2cc(N(c3ccc4ccccc4c3-c3ccc4ccccc4c3)c3cccc4sc5ccccc5c34)ccc2-c2cc3ccccc3cc21]

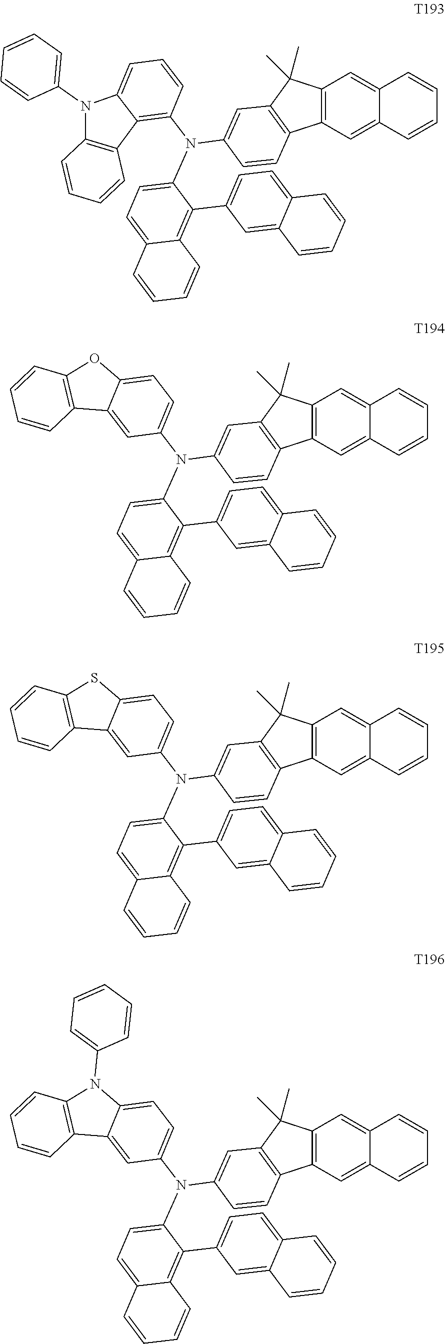 [SMILES: CC1(C)c2cc(N(c3ccc4ccccc4c3-c3ccc4ccccc4c3)c3cccc4c3c3ccccc3n4-c3ccccc3)ccc2-c2cc3ccccc3cc21 ; CC1(C)c2cc(N(c3ccc4oc5ccccc5c4c3)c3ccc4ccccc4c3-c3ccc4ccccc4c3)ccc2-c2cc3ccccc3cc21 ; CC1(C)c2cc(N(c3ccc4sc5ccccc5c4c3)c3ccc4ccccc4c3-c3ccc4ccccc4c3)ccc2-c2cc3ccccc3cc21 ; CC1(C)c2cc(N(c3ccc4c(c3)c3ccccc3n4-c3ccccc3)c3ccc4ccccc4c3-c3ccc4ccccc4c3)ccc2-c2cc3ccccc3cc21]

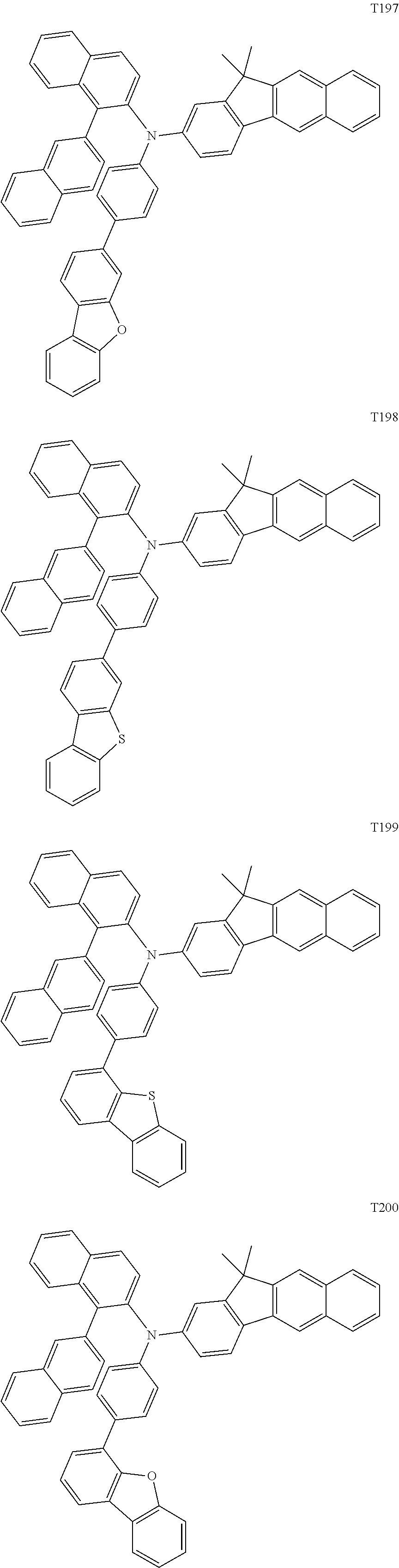 [SMILES: CC1(C)c2cc(N(c3ccc(-c4ccc5c(c4)oc4ccccc45)cc3)c3ccc4ccccc4c3-c3ccc4ccccc4c3)ccc2-c2cc3ccccc3cc21 ; CC1(C)c2cc(N(c3ccc(-c4ccc5c(c4)sc4ccccc45)cc3)c3ccc4ccccc4c3-c3ccc4ccccc4c3)ccc2-c2cc3ccccc3cc21 ; CC1(C)c2cc(N(c3ccc(-c4cccc5c4sc4ccccc45)cc3)c3ccc4ccccc4c3-c3ccc4ccccc4c3)ccc2-c2cc3ccccc3cc21 ; CC1(C)c2cc(N(c3ccc(-c4cccc5c4oc4ccccc45)cc3)c3ccc4ccccc4c3-c3ccc4ccccc4c3)ccc2-c2cc3ccccc3cc21]

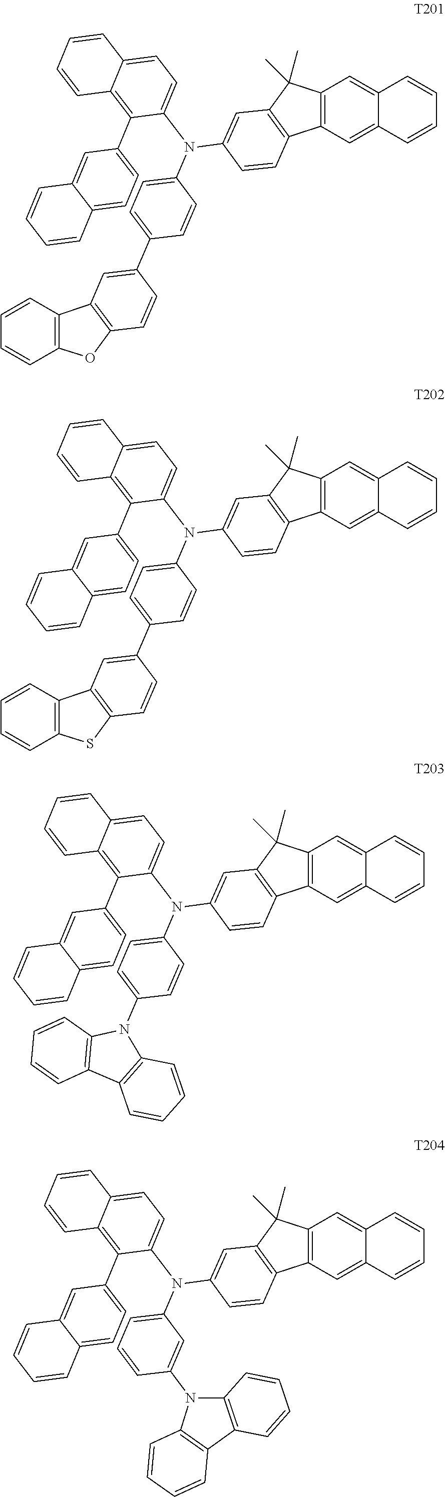 [SMILES: CC1(C)c2cc(N(c3ccc(-c4ccc5oc6ccccc6c5c4)cc3)c3ccc4ccccc4c3-c3ccc4ccccc4c3)ccc2-c2cc3ccccc3cc21 ; CC1(C)c2cc(N(c3ccc(-c4ccc5sc6ccccc6c5c4)cc3)c3ccc4ccccc4c3-c3ccc4ccccc4c3)ccc2-c2cc3ccccc3cc21 ; CC1(C)c2cc(N(c3ccc(-n4c5ccccc5c5ccccc54)cc3)c3ccc4ccccc4c3-c3ccc4ccccc4c3)ccc2-c2cc3ccccc3cc21 ; CC1(C)c2cc(N(c3cccc(-n4c5ccccc5c5ccccc54)c3)c3ccc4ccccc4c3-c3ccc4ccccc4c3)ccc2-c2cc3ccccc3cc21]

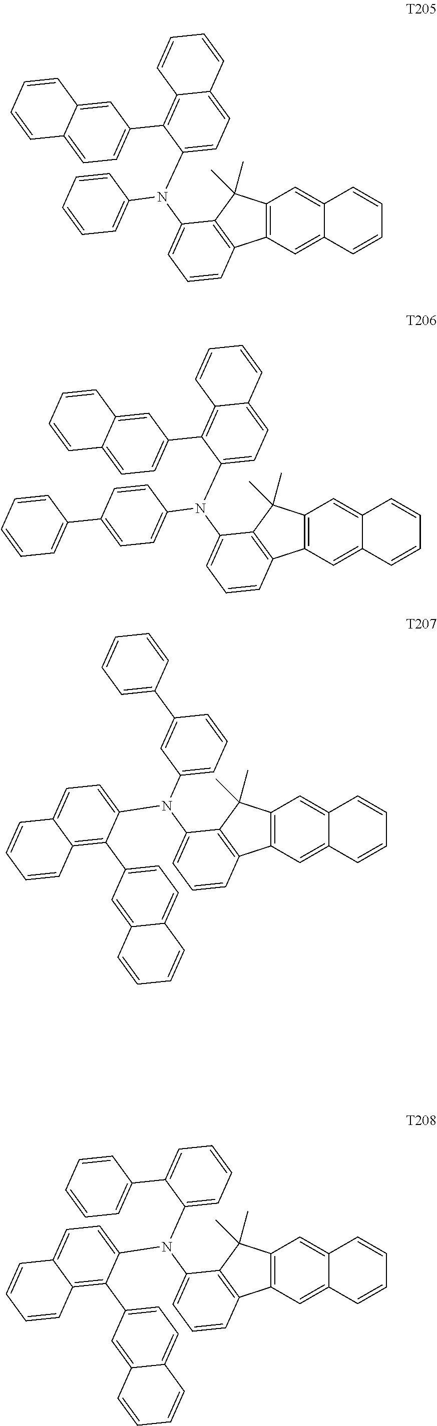 [SMILES: CC1(C)c2cc3ccccc3cc2-c2cccc(N(c3ccccc3)c3ccc4ccccc4c3-c3ccc4ccccc4c3)c21 ; CC1(C)c2cc3ccccc3cc2-c2cccc(N(c3ccc(-c4ccccc4)cc3)c3ccc4ccccc4c3-c3ccc4ccccc4c3)c21 ; CC1(C)c2cc3ccccc3cc2-c2cccc(N(c3cccc(-c4ccccc4)c3)c3ccc4ccccc4c3-c3ccc4ccccc4c3)c21 ; CC1(C)c2cc3ccccc3cc2-c2cccc(N(c3ccccc3-c3ccccc3)c3ccc4ccccc4c3-c3ccc4ccccc4c3)c21]

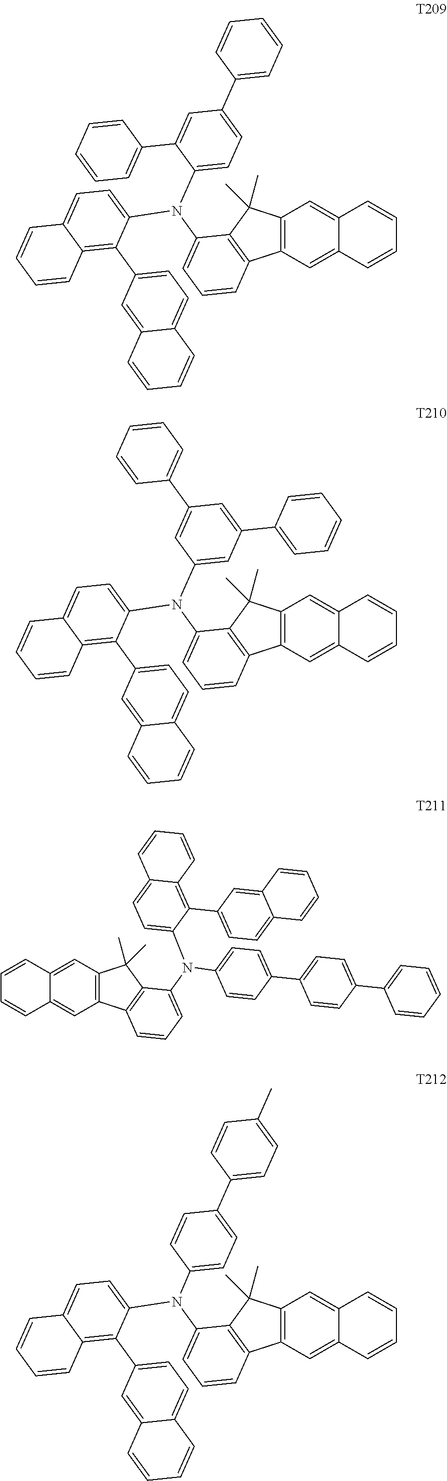

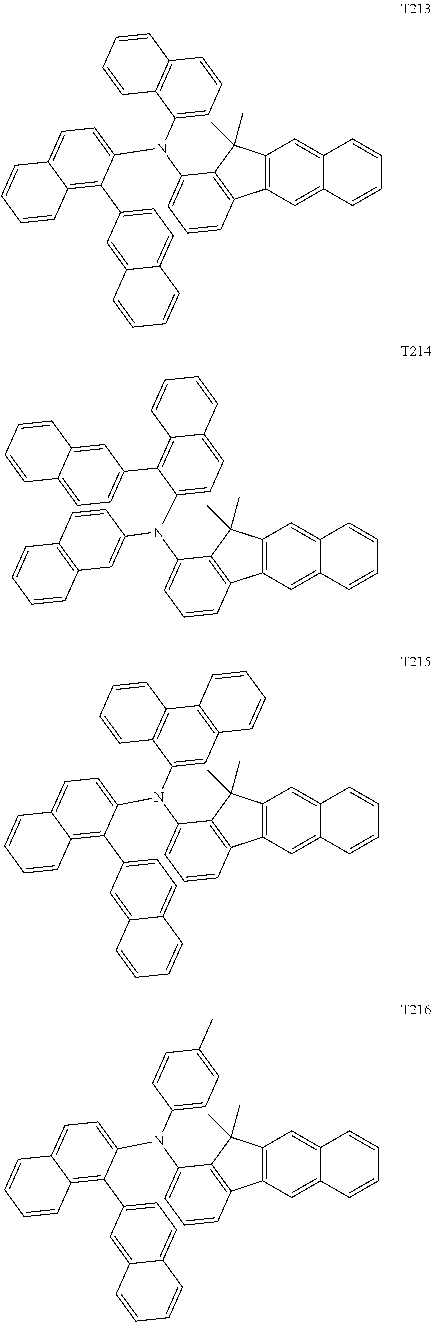 [SMILES: CC1(C)c2cc3ccccc3cc2-c2cccc(N(c3ccc4ccccc4c3-c3ccc4ccccc4c3)c3cccc4ccccc34)c21 ; CC1(C)c2cc3ccccc3cc2-c2cccc(N(c3ccc4ccccc4c3)c3ccc4ccccc4c3-c3ccc4ccccc4c3)c21 ; CC1(C)c2cc3ccccc3cc2-c2cccc(N(c3ccc4ccccc4c3-c3ccc4ccccc4c3)c3cc4ccccc4c4ccccc34)c21 ; Cc1ccc(N(c2cccc3c2C(C)(C)c2cc4ccccc4cc2-3)c2ccc3ccccc3c2-c2ccc3ccccc3c2)cc1]

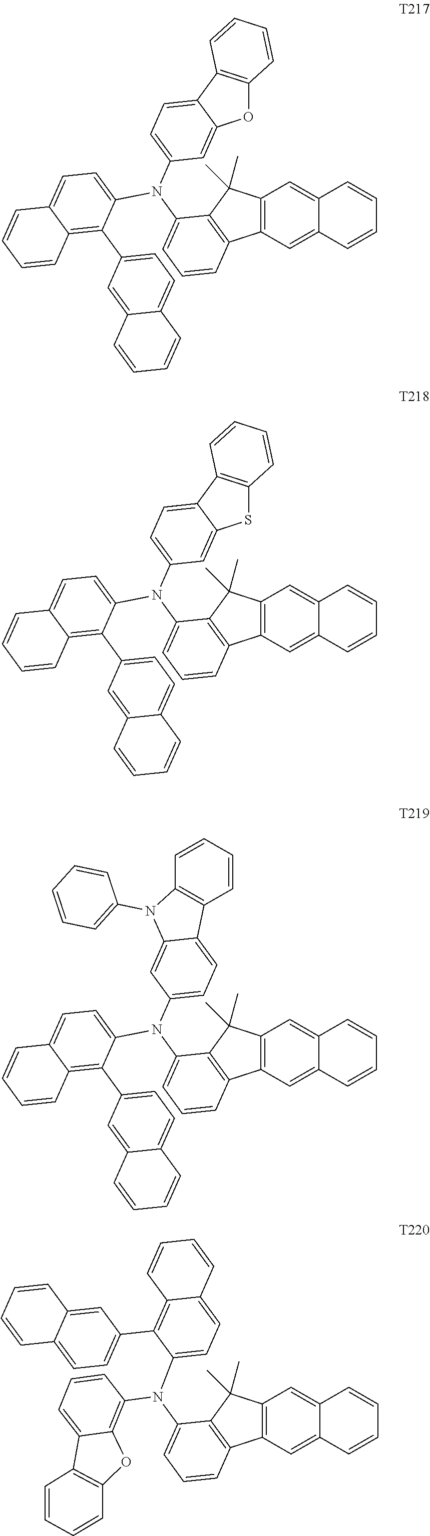

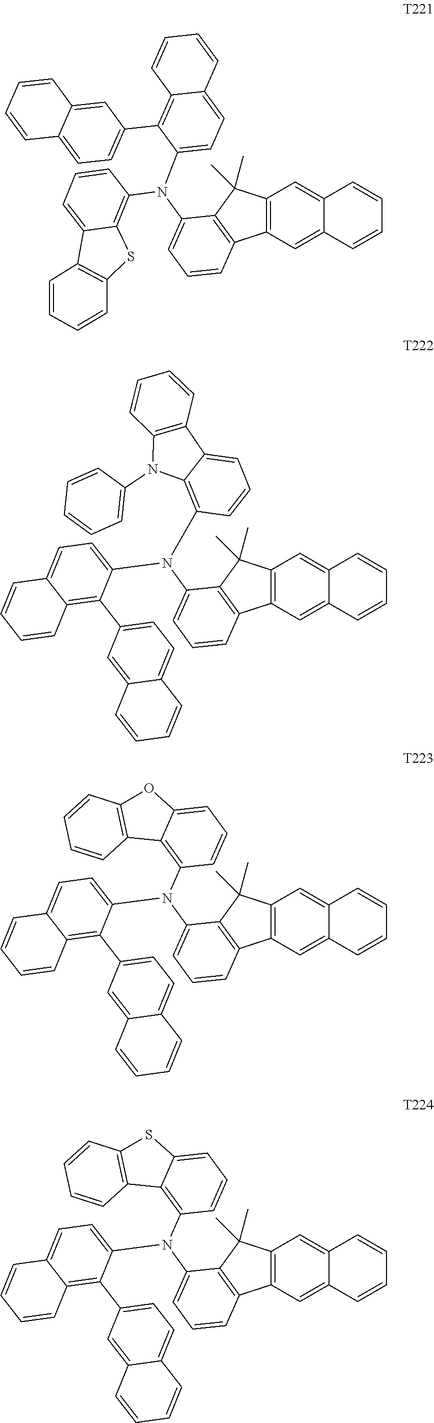 [SMILES: CC1(C)c2cc3ccccc3cc2-c2cccc(N(c3ccc4ccccc4c3-c3ccc4ccccc4c3)c3cccc4c3sc3ccccc34)c21 ; CC1(C)c2cc3ccccc3cc2-c2cccc(N(c3ccc4ccccc4c3-c3ccc4ccccc4c3)c3cccc4c5ccccc5n(-c5ccccc5)c34)c21 ; CC1(C)c2cc3ccccc3cc2-c2cccc(N(c3ccc4ccccc4c3-c3ccc4ccccc4c3)c3cccc4oc5ccccc5c34)c21 ; CC1(C)c2cc3ccccc3cc2-c2cccc(N(c3ccc4ccccc4c3-c3ccc4ccccc4c3)c3cccc4sc5ccccc5c34)c21]

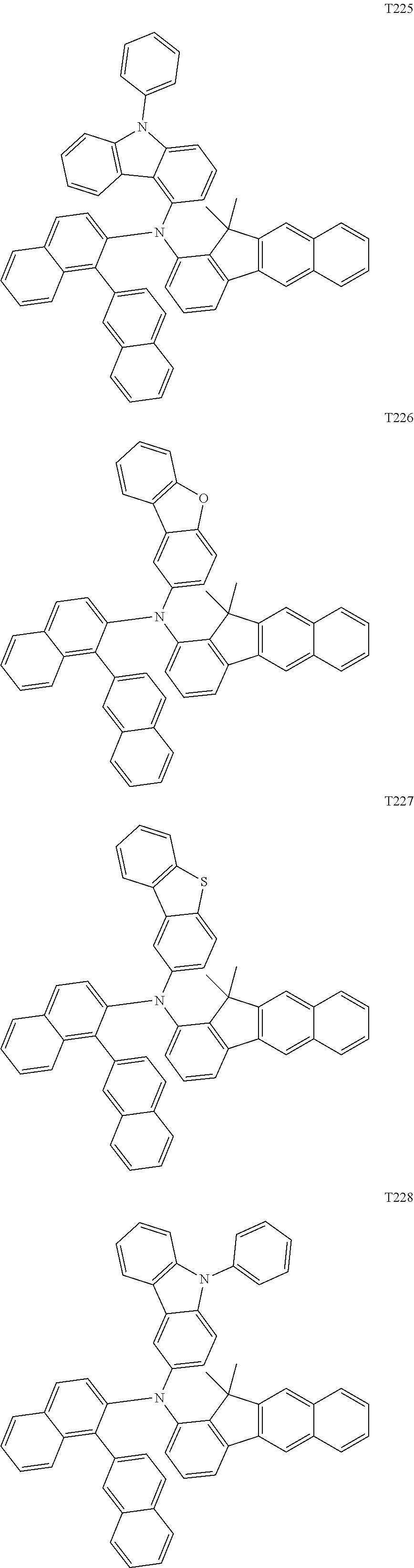

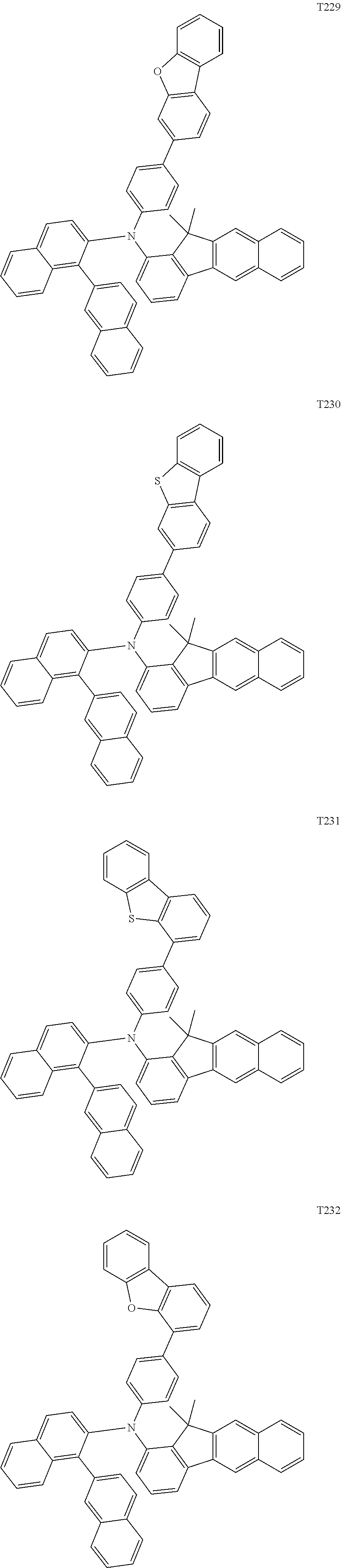 [SMILES: CC1(C)c2cc3ccccc3cc2-c2cccc(N(c3ccc(-c4ccc5c(c4)oc4ccccc45)cc3)c3ccc4ccccc4c3-c3ccc4ccccc4c3)c21 ; CC1(C)c2cc3ccccc3cc2-c2cccc(N(c3ccc(-c4ccc5c(c4)sc4ccccc45)cc3)c3ccc4ccccc4c3-c3ccc4ccccc4c3)c21 ; CC1(C)c2cc3ccccc3cc2-c2cccc(N(c3ccc(-c4cccc5c4sc4ccccc45)cc3)c3ccc4ccccc4c3-c3ccc4ccccc4c3)c21 ; CC1(C)c2cc3ccccc3cc2-c2cccc(N(c3ccc(-c4cccc5c4oc4ccccc45)cc3)c3ccc4ccccc4c3-c3ccc4ccccc4c3)c21]

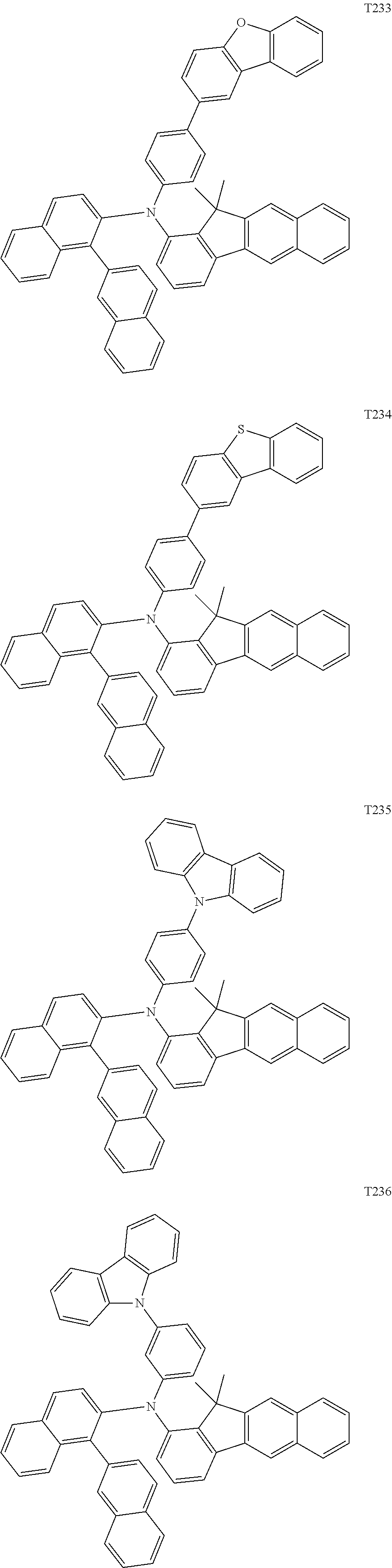 [SMILES: CC1(C)c2cc3ccccc3cc2-c2cccc(N(c3ccc(-c4ccc5oc6ccccc6c5c4)cc3)c3ccc4ccccc4c3-c3ccc4ccccc4c3)c21 ; CC1(C)c2cc3ccccc3cc2-c2cccc(N(c3ccc(-c4ccc5sc6ccccc6c5c4)cc3)c3ccc4ccccc4c3-c3ccc4ccccc4c3)c21 ; CC1(C)c2cc3ccccc3cc2-c2cccc(N(c3ccc(-n4c5ccccc5c5ccccc54)cc3)c3ccc4ccccc4c3-c3ccc4ccccc4c3)c21 ; CC1(C)c2cc3ccccc3cc2-c2cccc(N(c3cccc(-n4c5ccccc5c5ccccc54)c3)c3ccc4ccccc4c3-c3ccc4ccccc4c3)c21]

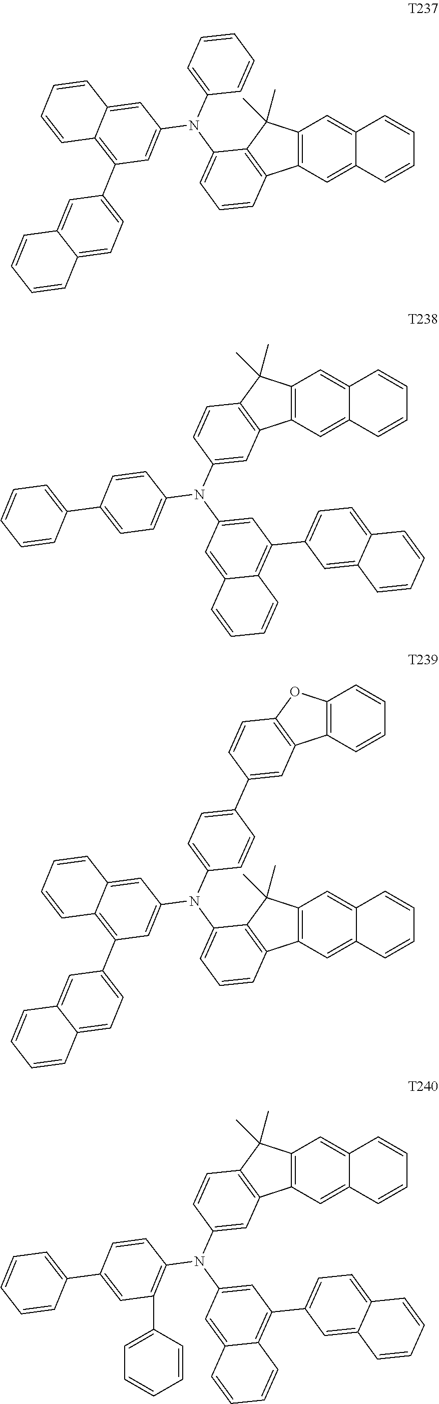 [SMILES: CC1(C)c2cc3ccccc3cc2-c2cccc(N(c3ccccc3)c3cc(-c4ccc5ccccc5c4)c4ccccc4c3)c21 ; CC1(C)c2ccc(N(c3ccc(-c4ccccc4)cc3)c3cc(-c4ccc5ccccc5c4)c4ccccc4c3)cc2-c2cc3ccccc3cc21 ; CC1(C)c2cc3ccccc3cc2-c2cccc(N(c3ccc(-c4ccc5oc6ccccc6c5c4)cc3)c3cc(-c4ccc5ccccc5c4)c4ccccc4c3)c21 ; CC1(C)c2ccc(N(c3cc(-c4ccc5ccccc5c4)c4ccccc4c3)c3ccc(-c4ccccc4)cc3-c3ccccc3)cc2-c2cc3ccccc3cc21]

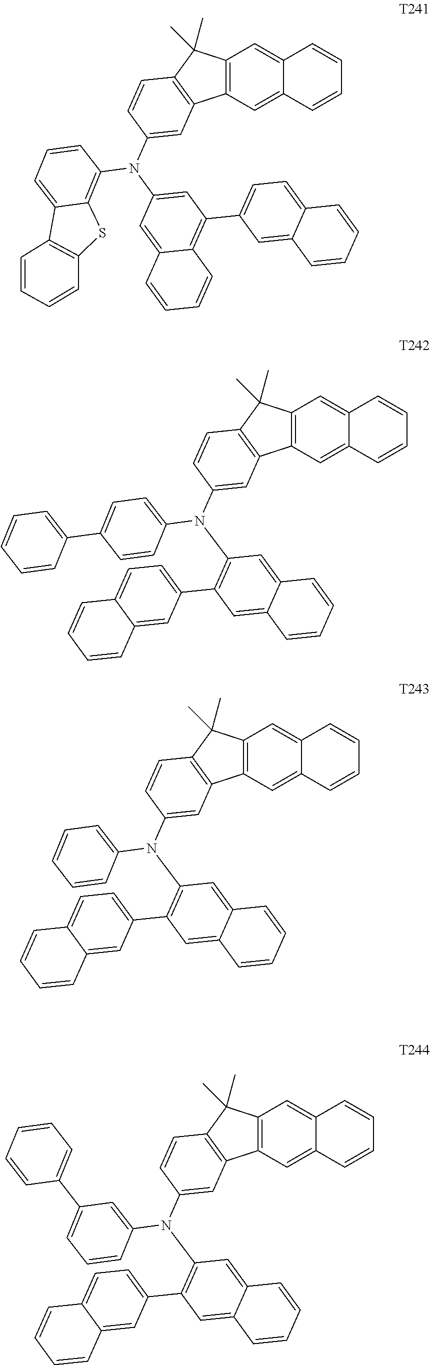 [SMILES: CC1(C)c2ccc(N(c3cc(-c4ccc5ccccc5c4)c4ccccc4c3)c3cccc4c3sc3ccccc34)cc2-c2cc3ccccc3cc21 ; CC1(C)c2ccc(N(c3ccc(-c4ccccc4)cc3)c3cc4ccccc4cc3-c3ccc4ccccc4c3)cc2-c2cc3ccccc3cc21 ; CC1(C)c2ccc(N(c3ccccc3)c3cc4ccccc4cc3-c3ccc4ccccc4c3)cc2-c2cc3ccccc3cc21 ; CC1(C)c2ccc(N(c3cccc(-c4ccccc4)c3)c3cc4ccccc4cc3-c3ccc4ccccc4c3)cc2-c2cc3ccccc3cc21]

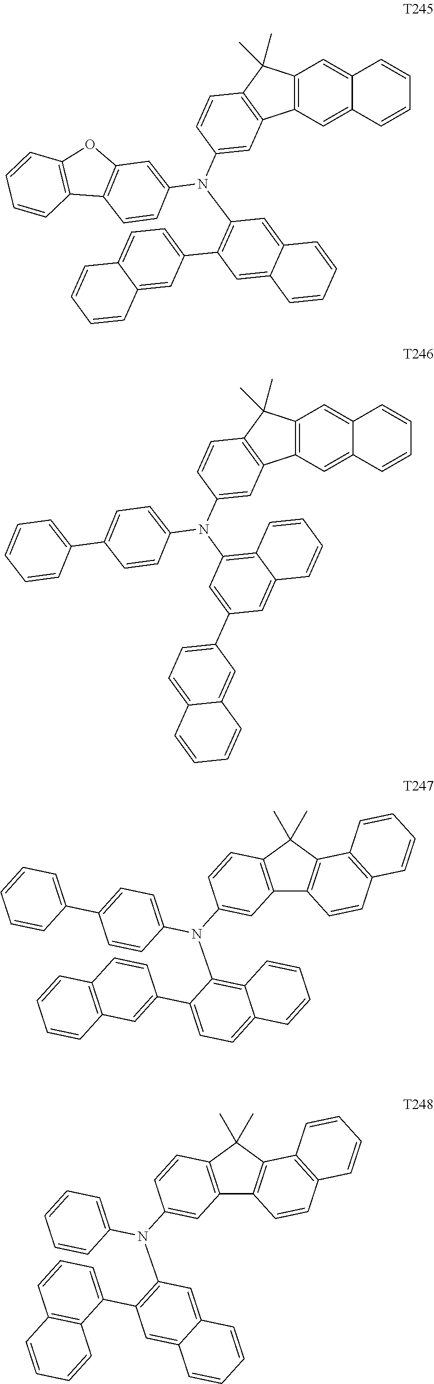 [SMILES: CC1(C)c2ccc(N(c3ccc4c(c3)oc3ccccc34)c3cc4ccccc4cc3-c3ccc4ccccc4c3)cc2-c2cc3ccccc3cc21 ; CC1(C)c2ccc(N(c3ccc(-c4ccccc4)cc3)c3cc(-c4ccc5ccccc5c4)cc4ccccc34)cc2-c2cc3ccccc3cc21 ; CC1(C)c2ccc(N(c3ccc(-c4ccccc4)cc3)c3c(-c4ccc5ccccc5c4)ccc4ccccc34)cc2-c2ccc3ccccc3c21 ; CC1(C)c2ccc(N(c3ccccc3)c3cc4ccccc4cc3-c3cccc4ccccc34)cc2-c2ccc3ccccc3c21]

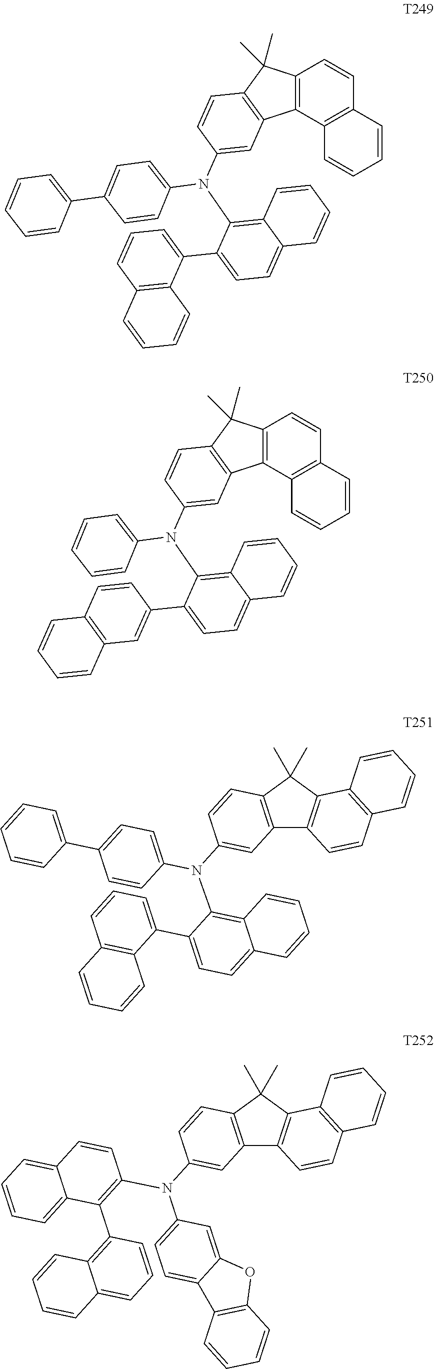 [SMILES: CC1(C)c2ccc(N(c3ccc(-c4ccccc4)cc3)c3c(-c4cccc5ccccc45)ccc4ccccc34)cc2-c2c1ccc1ccccc21 ; CC1(C)c2ccc(N(c3ccccc3)c3c(-c4ccc5ccccc5c4)ccc4ccccc34)cc2-c2c1ccc1ccccc21 ; CC1(C)c2ccc(N(c3ccc(-c4ccccc4)cc3)c3c(-c4cccc5ccccc45)ccc4ccccc34)cc2-c2ccc3ccccc3c21 ; CC1(C)c2ccc(N(c3ccc4c(c3)oc3ccccc34)c3ccc4ccccc4c3-c3cccc4ccccc34)cc2-c2ccc3ccccc3c21]

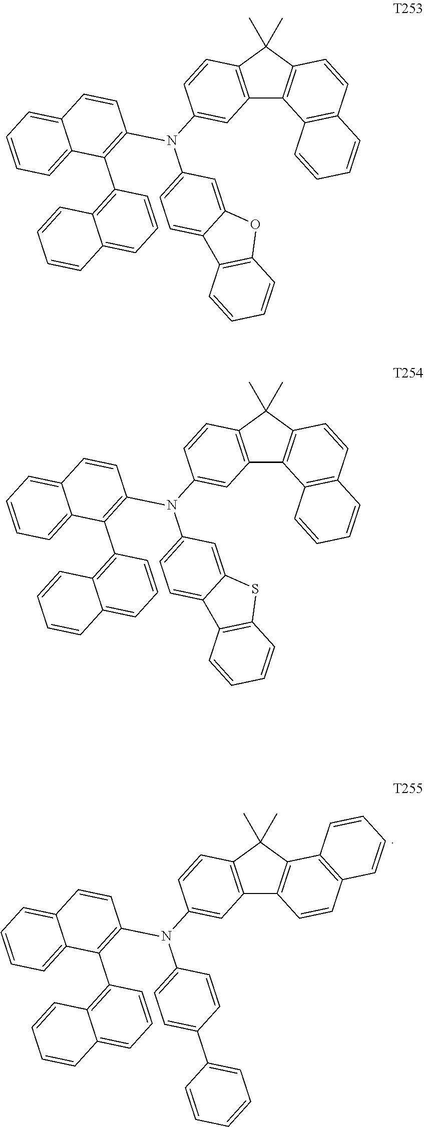 [SMILES: CC1(C)c2ccc(N(c3ccc4c(c3)oc3ccccc34)c3ccc4ccccc4c3-c3cccc4ccccc34)cc2-c2c1ccc1ccccc21 ; CC1(C)c2ccc(N(c3ccc4c(c3)sc3ccccc34)c3ccc4ccccc4c3-c3cccc4ccccc34)cc2-c2c1ccc1ccccc21 ; CC1(C)c2ccc(N(c3ccc(-c4ccccc4)cc3)c3ccc4ccccc4c3-c3cccc4ccccc34)cc2-c2ccc3ccccc3c21]

15. An application of the compound of claim 1 in an organic electroluminescent device, a lighting element, an organic thin film transistor, an organic field effect transistor, an organic thin film solar cell, an information label, an electronic artificial skin sheet, a sheet-type scanner, electronic paper or an organic EL panel, and preferably as a hole-transport material or an electron blocking material.

16. An organic electroluminescent device, comprising a substrate, a first electrode, a second electrode, and at least one organic layer located between the first electrode and the second electrode, wherein the organic layer comprises at least one compound of claim 1.

17. The organic electroluminescent device according to claim 16, wherein the organic layer comprises a hole transport region, and the hole transport region comprises the compound of claim 1;
preferably, the hole transport region comprises a hole transport layer and/or an electron blocking layer, wherein at least one of the hole transport layer and the electron blocking layer comprises the compound of claim 1.

18. An application of the compound of claim 6 in an organic electroluminescent device, a lighting element, an organic thin film transistor, an organic field effect transistor, an organic thin film solar cell, an information label, an electronic artificial skin sheet, a sheet-type scanner, electronic paper or an organic EL panel, and preferably as a hole-transport material or an electron blocking material.

19. An application of the compound of claim 9 in an organic electroluminescent device, a lighting element, an organic thin film transistor, an organic field effect transistor, an organic thin film solar cell, an information label, an electronic artificial skin sheet, a sheet-type scanner, electronic paper or an organic EL panel, and preferably as a hole-transport material or an electron blocking material.

20. An organic electroluminescent device, comprising a substrate, a first electrode, a second electrode, and at least one organic layer located between the first electrode and the second electrode, wherein the organic layer comprises at least one compound of claim 6.

21. An organic electroluminescent device, comprising a substrate, a first electrode, a second electrode, and at least one organic layer located between the first electrode and the second electrode, wherein the organic layer comprises at least one compound of claim 9.

22. The organic electroluminescent device according to claim 6, wherein the organic layer comprises a hole transport region, and the hole transport region comprises the compound of claim 6;
preferably, the hole transport region comprises a hole transport layer and/or an electron blocking layer, wherein at least one of the hole transport layer and the electron blocking layer comprises the compound of claim 6.

23. The organic electroluminescent device according to claim 9, wherein the organic layer comprises a hole transport region, and the hole transport region comprises the compound of claim 9;
preferably, the hole transport region comprises a hole transport layer and/or an electron blocking layer, wherein at least one of the hole transport layer and the electron blocking layer comprises the compound of claim 9.
